(19) United States
(12) Patent Application Publication
(10) Pub. No.: US 2024/0108630 A1
(43) Pub. Date: Apr. 4, 2024

(54) METHODS FOR INHIBITING RAS

(71) Applicants: The General Hospital Corporation, Boston, MA (US); Revolution Medicines, Inc., Redwood City, CA (US)

(72) Inventors: Ryan B. CORCORAN, Weston, MA (US); Robert J. NICHOLS, Redwood City, CA (US)

(21) Appl. No.: 18/479,500

(22) Filed: Oct. 2, 2023

Related U.S. Application Data
(63) Continuation of application No. PCT/US2022/023133, filed on Apr. 1, 2022.
(60) Provisional application No. 63/192,843, filed on May 25, 2021.
(60) Provisional application No. 63/170,292, filed on Apr. 2, 2021.

(51) Int. Cl. A61K 31/5377; A61K 31/517; A61K 31/519; A61P 35/00

(52) U.S. Cl. CPC A61K 31/5377; A61K 31/517; A61K 31/519; A61P 35/00

(57) ABSTRACT

The disclosure features methods for inhibiting RAS proteins, e.g., RAS proteins that have acquired resistance to one or more RAS inhibitors. The disclosure also methods for the treatment of cancer.

### CLAIM OF PRIORITY

The present application is a continuation application of International Patent Application Serial No. PCT/US2022/023133, filed on Apr. 1, 2022, which claims the benefit of priority to U.S. Provisional Patent Application Ser. Nos. 63/170,292, filed on Apr. 2, 2021, and 63/192,843, filed on May 25, 2021, the entire contents of which are hereby incorporated by reference.

### BACKGROUND

Cancer remains one of the most-deadly threats to human health. In the U.S., cancer affects nearly 1.3 million new patients each year, and is the second leading cause of death after heart disease, accounting for approximately 1 in 4 deaths.

It has been well established in literature that RAS proteins (KRAS, HRAS, and NRAS) play an essential role in various human cancers and are therefore appropriate targets for anticancer therapy. Indeed, mutations in RAS proteins account for approximately 30% of all human cancers in the United States, many of which are fatal. Dysregulation of RAS proteins by activating mutations, overexpression, or upstream activation is common in human tumors, and activating mutations in RAS are frequently found in human cancer. RAS converts between a GDP-bound “off” and a GTP-bound “on” state. The conversion between states is facilitated by interplay between a guanine nucleotide exchange factor (GEF) protein (e.g., SOS1), which loads RAS with GTP, and a GTPase-activating protein (GAP) protein (e.g., NF1), which hydrolyzes GTP, thereby inactivating RAS. Additionally, the SH2 domain-containing protein tyrosine phosphatase-2 (SHP2) associates with the receptor signaling apparatus and becomes active upon RTK activation, and then promotes RAS activation. Mutations in RAS proteins can lock the protein in the “on” state resulting in a constitutively active signaling pathway that leads to uncontrolled cell growth. For example, activating mutations at codon 12 in RAS proteins function by inhibiting both GAP-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codons 13 (e.g., G13D) and 61 (e.g., Q61K) of RAS are also responsible for oncogenic activity in some cancers.

First-in-class covalent inhibitors of the “off” form of RAS (RAS(OFF)) have demonstrated promising anti-tumor activity in cancer patients with oncogenic mutations in RAS. Further, therapeutic inhibition of the RAS pathway, although often initially efficacious, can ultimately prove ineffective as it may lead to over-activation of RAS pathway signaling via a number of mechanisms including, e.g., reactivation of the pathway via relief of the negative feedback machineries that naturally operate in these pathways. As a result, cells that were initially sensitive to such inhibitors may become resistant. Thus, a need exists for methods of effectively inhibiting RAS pathway signaling while minimizing or mitigating activation of resistance mechanisms.

### SUMMARY

The present disclosure provides methods for inhibiting RAS and for the treatment of cancer. The inventors observed that cancer cells treated with a RAS(OFF) inhibitor may develop resistance, e.g., through the acquisition of one or more mutations that render the RAS(OFF) inhibitor less effective or ineffective. The disclosure is based, at least in part, on the observation that some cancers resistant to treatment with a RAS(OFF) inhibitor remain responsive to treatment with a RAS(ON) inhibitor. Thus, administering a RAS(ON) inhibitor to a subject having cancer can slow or halt oncogenic signaling or disease progression where the cancer is resistant to treatment with a RAS(OFF) inhibitor. Additionally, administration of a RAS(ON) inhibitor, e.g., administered in combination with a RAS(OFF) inhibitor, may prevent the acquisition of one or more mutations in RAS that confer resistance to the RAS(OFF) inhibitor.

Accordingly, in a first aspect, the disclosure provides a method of treating cancer in a subject in need thereof, wherein the cancer includes a mutation in RAS and the cancer is resistant to treatment with a RAS(OFF) inhibitor, the method including administering to the subject a RAS(ON) inhibitor. In some embodiments, the RAS mutation is an amino acid substitution at Y96. In some embodiments, the amino acid substitution is Y96D.

In another aspect, the disclosure provides a method of treating cancer in a subject in need thereof, wherein the cancer includes an amino acid substitution at RAS Y96, the method including administering to the subject a RAS(ON) inhibitor. In some embodiments, the amino acid substitution is Y96D.

In some embodiments, the method further includes administering to the subject a RAS(OFF) inhibitor (e.g., a RAS(OFF) inhibitor is administered to the subject in combination with the RAS(ON) inhibitor). The RAS(ON) inhibitor and the RAS(OFF) inhibitor may be administered simultaneously or sequentially. The RAS(ON) inhibitor and the RAS(OFF) inhibitor may administered as a single formulation or in separate formulations. In some embodiments, the RAS(OFF) inhibitor is administered for a first period of time; and the RAS(ON) inhibitor is administered for a second period of time, wherein the first period of time and the second period of time do not overlap and the first period of time precedes the second period of time. In some embodiments, the RAS(OFF) inhibitor is administered for a first period of time; and the RAS(OFF) inhibitor and RAS(ON) inhibitor are administered for a second period of time, wherein the first period of time and the second period of time do not overlap and the first period of time precedes the second period of time. In some embodiments, the first period of time is a period of time sufficient to acquire a mutation (e.g., a RAS mutation) that confers resistance to treatment with the RAS(OFF) inhibitor. In some embodiments, the first period of time is between one week and one month, between one week and six months, between one week and one year, between one month and six months, between one month and one year, between one month and two years, between one month and five years, at least one week, at least one month, at least six months, or at least one year. In some embodiments, the second period of time is between one week and one month, between one week and six months, between one week and one year, between one month and six months, between one month and one year, between one month and two years, between one month and five years, at least one week, at least one month, at least six months, or at least one year.

In some embodiments, the subject's cancer progresses on the RAS(OFF) inhibitor (e.g., when the subject is administered the RAS(OFF) inhibitor in the absence of a RAS(ON) inhibitor).

In some embodiments, the subject has been treated with a RAS(OFF) inhibitor (e.g., the subject has been previously treated with a RAS(OFF) inhibitor, e.g., prior to administration of the RAS(ON) inhibitor). In some embodiments, the subject has acquired resistance to a RAS(OFF) inhibitor (e.g., has acquired a mutation that confers resistance to a RAS(OFF) inhibitor, e.g., prior to administration of the RAS(ON) inhibitor).

In another aspect, the disclosure provides a method of inhibiting RAS in a cell, wherein the RAS includes an amino acid substitution at Y96, the method including contacting the cell with a RAS(ON) inhibitor. In some embodiments, the amino acid substitution is Y96D.

In some embodiments, the RAS includes or further includes an amino acid substitution at G12, G13, Q61, or a combination thereof. In some embodiments, the amino acid substitution is selected from G12C, G12D, G12V, G13C, G13D, or Q61L. In some embodiments, the amino acid substitution is G12C.

In some embodiments, the RAS is KRAS. In some embodiments, the KRAS includes or further includes an amino acid substitution at G12, G13, Q61, A146, K117, L19, Q22, V14, A59, or a combination thereof. In some embodiments, the KRAS amino acid substitution is selected from G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, G13V, or a combination thereof.

In some embodiments, the RAS is NRAS. In some embodiments, the NRAS includes or further includes an amino acid substitution at G12, G13, Q61, P185, A146, G60, A59, E132, E49, T50, or a combination thereof. In some embodiments, the NRAS amino acid substitution is selected from Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, A59T, or a combination thereof.

In some embodiments, the RAS is HRAS. In some embodiments, the HRAS includes or further includes an amino acid substitution at G12, G13, Q61, K117, A59, A18, D119, A66, A146, or a combination thereof. In some embodiments, the HRAS amino acid substitution is selected from Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, G12R, or a combination thereof.

In some embodiments, the RAS(ON) inhibitor is an inhibitor selective for RAS G12C, G13D, or G12D. In some embodiments, the RAS(ON) inhibitor is a RAS(ON)^{MULTI} inhibitor.

In some embodiments, the RAS(ON) inhibitor is a compound described by Formula AI:

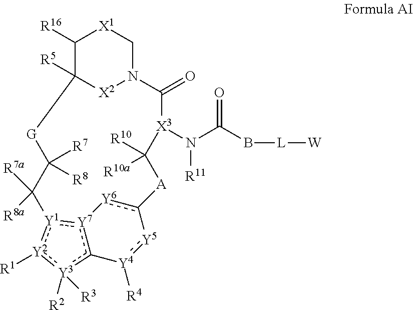

or a pharmaceutically acceptable salt thereof,
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl (e.g., methyl).

In some embodiments, the RAS(ON) inhibitor is a compound, or a pharmaceutically acceptable salt thereof, of any one of Formula Ala, Formula Alb, Formula Alc, Formula Ald, Formula Ale, Formula Alf, Formula Alg, Formula Alh, or Formula Ali described herein.

In some embodiments, the RAS(ON) inhibitor is selected from a compound of Table A1 or Table A2, or a pharmaceutically acceptable salt thereof.

In some embodiments, the RAS(ON) inhibitor is a compound of Formula BI:

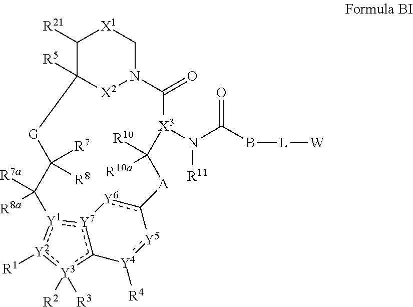

or a pharmaceutically acceptable salt thereof,
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y3, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl.

In some embodiments, the RAS(ON) inhibitor is a compound, or a pharmaceutically acceptable salt thereof, of any one of Formula Bla, Formula Bib, Formula Blc, Formula Bld, Formula Ble, Formula Bif, Formula Big, Formula BVI, Formula BVia, Formula BVIb, or Formula BVic described herein.

In some embodiments, the RAS(ON) inhibitor is selected from a compound of Table B1 or Table B2, or a pharmaceutically acceptable salt thereof.

In some embodiments, the RAS(ON) inhibitor is a compound described by Formula CI:

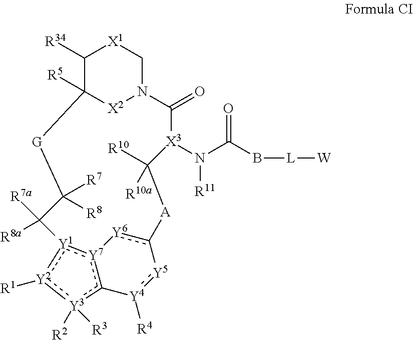

or a pharmaceutically acceptable salt thereof,
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene; G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y3, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl; and
R³⁴ is hydrogen or C₁-C₃ alkyl.

In some embodiments, the RAS(ON) inhibitor is a compound, or a pharmaceutically acceptable salt thereof, of any one of Formula Cla, Formula Cib, Formula Cic, Formula Cld, Formula Cle, Formula Clf, Formula CVI, Formula CVla, CFormula Vib, or Formula CVII described herein.

In some embodiments, the RAS(ON) inhibitor is selected from a compound of Table C1 or Table C₂, or a pharmaceutically acceptable salt thereof.

In some embodiments, the RAS(ON) inhibitor is a compound described by Formula Dla:

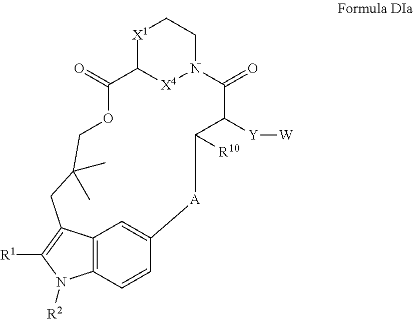

or a pharmaceutically acceptable salt thereof,
wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;

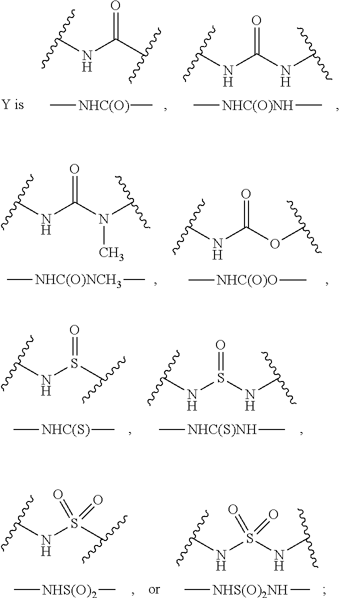

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R¹⁰ is hydrogen, hydroxy, optionally substituted C₁-C₃ alkyl, or optionally substituted C₁-C₆ heteroalkyl.

In some embodiments, the RAS(ON) inhibitor is a compound, or a pharmaceutically acceptable salt thereof, of any one of Formula DII (e.g., Formula DII-1, DII-2, DII-3, DII-4, DII-5, DII-6, DII-7, DII-8, or DII-9), Formula DIII (e.g., Formula DIII-1, DIII-2, DIII-3, DIII-4, DIII-5, DIII-6, DIII-7, DIII-8, or DIII-9), Formula DIV (e.g., Formula DIV-1, DIV-2, DIV-3, DIV-4, DIV-5, DIV-6, DIV-7, DIV-8, or DIV-9), Formula DV (e.g., Formula DV-1, DV-2, DV-3, DV-4, or DV-5), Formula DVI (e.g., Formula DVI-1, DVI-2, DVI-3, DVI-4, or DVI-5), Formula DVII (e.g., Formula DVII-1, DVII-2, DVII-3, DVII-4, or DVII-5), Formula DVIII (e.g., Formula DVIII-1, DVIII-2, DVIII-3, DVIII-4, or DVIII-5), Formula DIX (e.g., Formula DIX-1, DIX-2, DIX-3, DIX-4, or DIX-5), or Formula DX (e.g., Formula DX-1, DX-2, DX-3, DX-4, or DX-5)

In some embodiments, the RAS(ON) inhibitor is selected from a compound of Table D1a or D1 b, or a pharmaceutically acceptable salt thereof.

In some embodiments, the RAS(ON) inhibitor is a compound described by a Formula in WO 2020132597, such as a compound of Formula (I) therein, or a pharmaceutically acceptable salt thereof, or a compound of FIG. 1 therein, or a pharmaceutically acceptable salt thereof.

In some embodiments, the RAS(OFF) inhibitor selectively targets RAS G12C. In some embodiments, the RAS(OFF) inhibitor selectively targets RAS G12D.

In some embodiments, the RAS(OFF) inhibitor is selected from AMG 510 (sotorasib), MRTX (adagrasib), MRTX1257, JNJ-74699157 (ARS-3248), LY3537982, LY3499446, ARS-853, ARS-1620, GDC-6036, JDQ443, BPI-421286, and JAB-21000. In some embodiments, the RAS(OFF) inhibitor is an inhibitor of K-Ras G12D, such as MRTX1133 or JAB-22000. In some embodiments, the RAS(OFF) inhibitor is a K-Ras G12V inhibitor, such as JAB-23000.

In some embodiments, the RAS(OFF) inhibitor is a compound disclosed in any one of the following patent publications: WO 2022052895, WO 2022048545, WO 2022047093, WO 2022042630, WO 2022040469, WO 2022037631, WO 2022037560, WO 2022031678, WO 2022028492, WO 2022028346, WO 2022026726, WO 2022026723, WO 2022015375, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, CN 114195788, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113248521, CN 113087700, CN 113024544, CN 113004269, CN 112920183, CN 112390818, CN 112390788, CN 112300194, CN 112300173, CN 112225734, CN 112142735, CN 112110918, CN 112094269, CN 112047937, and CN 109574871, each of which is incorporated herein by reference in its entirety.

In any embodiment herein regarding a RAS(OFF) inhibitor, the RAS(OFF) inhibitor may be substituted by a RAS inhibitor disclosed in the following patent publication: WO 2021041671, which is incorporated herein by reference in its entirety. In some embodiments, such a substituted RAS inhibitor is MRTX1133.

In some embodiments, the cancer is selected from colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, bladder cancer, appendiceal cancer, endometrial cancer, and melanoma. In some embodiments, the cancer is non-small cell lung cancer. In some embodiments, the cancer is pancreatic cancer.

It is specifically contemplated that any limitation discussed with respect to one embodiment of the disclosure may apply to any other embodiment of the disclosure. Furthermore, any compound or composition of the disclosure may be used in any method of the disclosure, and any method of the disclosure may be used to produce or to utilize any compound or composition of the disclosure.

### BRIEF DESCRIPTION OF THE FIGURES

FIG. 1A is a series of computed tomography (CT) images of a subject's axillary lymph node metastasis at baseline, during response to a RAS(OFF) inhibitor, MRTX849, and at progression on MRTX849.

FIG. 1B is a western blot analysis of MIA PaCa-2 cells (stably expressing BRAF (V600E)-V5) that were treated with a RAS(OFF) inhibitor, MRTX849, at the indicated concentrations for 4 hours.

FIG. 1C is a diagram illustrating alterations detected in post-MRTX849 cfDNA that include acquired mutations in KRAS as well as multiple components of the MAPK signaling cascade.

FIG. 2A is a sequence read pile-up of KRAS^{G13D} occurring in trans to KRAS^{G12C}.

FIG. 2B is a sequence read pile-up of KRAS^{G12V} occurring in cis to KRAS^{G12C}.

FIG. 3 is a series of modeled crystal structures of RAS(OFF) inhibitors MRTX849 (6UT0), AMG 510 (6OIM), and ARS-1620 (5V9U) bound to KRAS^{G12C} (top panels) and KRAS^{G12C/Y96D} (bottom panels).

FIG. 4A are a series of plots of cell viability assays performed with NCI-H358, MIA PaCa-2 and Ba/F3 cells infected with retrovirus packaging KRAS (G12C or G12C/Y96D) in the presence of RAS(OFF) inhibitors.

FIG. 4B is a Western blot analysis of MIA PaCa-2 cells stably expressing KRAS^{G12C} or KRAS^{G12C/Y96D} that were treated with a RAS(OFF) inhibitor, MRTX849 for 4 hours.

FIG. 4C is a Western blot of MGH1138-1 cells expressing KRAS^{G12C} or KRAS^{G12C/Y96D} after treatment with a RAS(OFF) inhibitor, MRTX849, for 4 hours. Cell viability data of the MGH1138-1 cells is plotted on the right following 72 hours of treatment with the indicated concentrations of MRTX849.

FIG. 4D is a Western blot of HEK293T cells transiently expressing KRAS mutants after treatment with a RAS(OFF) inhibitor, MRTX849, for 4 hours.

FIG. 4E is a bar graph showing densitometry analysis of KRAS-GTP levels of untreated HEK293T stably expressing KRAS^{G12C} and KRAS^{G12C/Y96D}.

FIG. 4F is a Western blot analysis of HEK293T stably expressing KRAS mutants treated with indicated inhibitors for 4 h.

FIG. 4G is a RAS-GTP pulldown assay performed after treating HEK293T stably expressing KRAS mutants in the presence of a RAS(OFF) inhibitor, MRTX849, for 4 hours.

FIG. 4H is a Western blot of LU-65 cells transiently expressing KRAS^{G12C} or KRAS^{G12C/Y96D} after treatment with MRTX849 for 4 hours.

FIG. 5A illustrates the mechanism of action of a RAS(ON) inhibitor, RM-018, which is a RAS(ON)G12C inhibitor compound of Formula BI herein, and also a compound of Table B1 herein, and is also found in WO 2021/091982.

FIG. 5B is a graph of cell viability of cells harboring various mutations in the presence of a RAS(ON) inhibitor, RM-018.

FIG. 5C is a series of cell viability plots performed with NCI-H358, MIA PaCa-2, Ba/F3 and MGH1138-1 cells stably infected with KRAS^{G12C} or KRAS^{G12C/Y96D} treated for 72 hours with a RAS(ON) inhibitor, RM-018.

FIG. 5D is a Western blot analysis performed in MIA PaCa-2 stably expressing KRAS^{G12C} or KRAS^{G12C/Y96D} after treatment with a RAS(ON) inhibitor, RM-018, for 4 hours.

FIG. 5E is a Western blot analysis of HEK293T cells transiently expressing the indicated KRAS mutant after treatment with a RAS(ON) inhibitor, RM-018 for 4 hours.

FIG. 5F is a Western blot analysis of MGH1138-1 cells transiently expressing KRAS^{G12C} or KRAS^{G12C/Y96D} after treatment with a RAS(ON) inhibitor, RM-018, for 4 hours.

FIG. 5G is a Western blot performed with HEK293T cells transiently expressing KRAS mutants after being treated with the indicated drug at 100 nmol/L each for 4 hours.

FIG. 6 is a graph showing that compound AA, a KRAS^{G12C}(ON) inhibitor, which is a RAS(ON)G12C inhibitor compound of Formula BI herein, and also a compound of Table B1 herein, and is also found in WO 2021/091982, inhibits KRAS^{G12C/Y96D} in cells.

FIG. 7 is a graph showing pERK potency of Compound AA, a KRAS^{G12C}(ON) inhibitor, in KRAS^{G12C/Y96D} cells.

### DETAILED DESCRIPTION

The present disclosure relates generally to methods for inhibiting RAS and for the treatment of cancer. In some embodiments, the disclosure provides methods for delaying, preventing, or treating acquired resistance to a RAS(OFF) inhibitor by administering a RAS(ON) inhibitor. In some embodiments, administration of a RAS(ON) inhibitor, e.g., administered in combination with a RAS(OFF) inhibitor, may prevent the acquisition of one or more mutations in RAS that confers resistance to the RAS(OFF) inhibitor.

### General Methods

The practice of the present disclosure will employ, unless otherwise indicated, conventional techniques of cell culturing, molecular biology (including recombinant techniques), microbiology, cell biology, biochemistry, and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C ¹³C ¹⁴C ¹³N, ¹⁵N, ¹⁵O, ¹⁷O, ¹⁸O, ³²P, ³³P, ³⁵S, ¹⁸F, ³⁶C, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present disclosure described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O )NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R^{∘})—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)_{0- 4}OC(O) NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)_{0- 4}S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O )(OR^{∘})R^{∘}, —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)_{0- 2}NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{*}₂, ═NNHC(O)R^{*}, ═NNHC(O)OR^{*}, ═NNHS(O)₂R^{*}, =NR^{*}, =NOR^{*}, —O(C(R^{*}₂))₂₋₃O—, or —S(C(R^{*}₂))₂₋₃S—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{*} include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR†, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}2, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include═O and ═S.

The term “acetyl,” as used herein, refers to the group —C(O)CH₃.

As used herein, the term “administration” refers to the administration of a composition (e.g., a compound, or a preparation that includes a compound as described herein) to a subject or system. Administration also includes administering a prodrug derivative or analog of the compound or pharmaceutically acceptable salt of the compound or composition to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

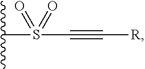

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

An “amino acid substitution,” as used herein, refers to the substitution of a wild-type amino acid of a protein with a non-wild-type amino acid. Amino acid substitutions can result from genetic mutations and may alter one or more properties of the protein (e.g., may confer altered binding affinity or specificity, altered enzymatic activity, altered structure, or altered function). For example, where a RAS protein includes an amino acid substitution at position Y96, this notation indicates that the wild-type amino acid at position 96 of the RAS protein is a Tyrosine (Y), and that the RAS protein including the amino acid substitution at position Y96 includes any amino acid other than Tyrosine (Y) at position 96. The notation Y96D indicates that the wild-type Tyrosine (Y) residue at position 96 has been substituted with an Aspartic Acid (D) residue.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “combination therapy” refers to a method of treatment including administering to a subject at least two therapeutic agents, optionally as one or more pharmaceutical compositions, as part of a therapeutic regimen. For example, a combination therapy may include administration of a single pharmaceutical composition including at least two therapeutic agents and one or more pharmaceutically acceptable carrier, excipient, diluent, or surfactant. A combination therapy may include administration of two or more pharmaceutical compositions, each composition including one or more therapeutic agent and one or more pharmaceutically acceptable carrier, excipient, diluent, or surfactant. In various embodiments, at least one of the therapeutic agents is a RAS(ON) inhibitor (e.g., any one or more RAS(ON) inhibitors (e.g., KRAS(ON) inhibitors) disclosed herein or known in the art). In various embodiments, at least one of the therapeutic agents is a RAS(OFF) inhibitor (e.g., any one or more RAS(OFF) inhibitors (e.g., KRAS(OFF) inhibitors) disclosed herein or known in the art). The two or more agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due to an additive or synergistic effect of combining the two or more therapeutics.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

As used herein, the term “dosage form” refers to a physically discrete unit of a compound (e.g., a compound of the present disclosure) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present disclosure) has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The term “disorder” is used in this disclosure to mean, and is used interchangeably with, the terms disease, condition, or illness, unless otherwise indicated.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

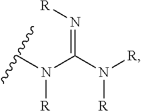

wherein each R is, independently, any any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. Small molecule inhibitors include cyclic and acyclic compounds. Small molecules inhibitors include natural products, derivatives, and analogs thereof. Small molecule inhibitors can include a covalent cross-linking group capable of forming a covalent cross-link, e.g., with an amino acid side-chain of a target protein.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting a first moiety (e.g., a macrocyclic moiety or B) to a second moiety (e.g., W) in a compound of any one of Formula A1, Formula BI, Formula CI, Formula DIA, or a subformula thereof, such that the resulting compound is capable of achieving an IC50 of 2 uM or less in the Ras-RAF disruption assay protocol provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu-W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, μm 665/615 nm). Compounds that facilitate disruption of a Ras:RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “mutation” as used herein indicates any modification of a nucleic acid or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications or chromosomal breaks or translocations. In particular embodiments, the mutation results in an amino acid substitution in the encoded-protein.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The term “preventing acquired resistance,” as used herein, means avoiding the occurrence of acquired or adaptive resistance. For example, the use of a RAS(ON) inhibitor described herein in preventing acquired/adaptive resistance to a RAS(OFF) inhibitor means that the RAS(ON) inhibitor is administered prior to any detectable existence of resistance to the RAS(OFF) inhibitor and the result of such administration of the RAS(ON) inhibitor is that no resistance to the RAS(OFF) inhibitor occurs.

As used herein, the term “pharmaceutical composition” refers to a compound, such as a compound of the present disclosure, or a pharmaceutically acceptable salt thereof, formulated together with a pharmaceutically acceptable excipient.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The terms “RAS inhibitor” and “inhibitor of [a] RAS” are used interchangeably to refer to any inhibitor that targets, that is, selectively binds to or inhibits a RAS protein. In various embodiments, these terms include RAS(OFF) and RAS(ON) inhibitors.

As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). RAS(ON) inhibitors described herein include compounds of Formula AI, Formula BI, Formula CI, Formula DIa, and subformula thereof, and compounds of Table A1, Table A2, Table B1, Table B2, Table C1, Table C2, Table D1a, Table D1 b, Table D2, Table D3, as well as salts (e.g., pharmaceutically acceptable salts), solvates, hydrates, stereoisomers (including atropisomers), and tautomers thereof.

As used herein, the term “RAS(OFF) inhibitor” refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS).

As used herein, the term “RAS(ON)^{MULTI} inhibitor” refers to a RAS(ON) inhibitor of at least 3 RAS variants with missense mutations at one of the following positions: 12, 13, 59, 61, or 146. In some embodiments, a RAS(ON)^{MULTI} inhibitor refers to a RAS(ON) inhibitor of at least 3 RAS variants with missense mutations at one of the following positions: 12, 13, and 61.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

As used herein, the term “resistant to treatment” refers to a treatment of a disorder with a therapeutic agent, where the therapeutic agent is ineffective or where the therapeutic agent was previously effective and has become less effective over time. Resistance to treatment includes acquired resistance to treatment, which refers to a decrease in the efficacy of a treatment over a period of time where the subject is being administered the therapeutic agent. Acquired resistance to treatment may result from the acquisition of a mutation in a target protein that renders the treatment ineffective or less effective. Accordingly, resistance to treatment may persist even after cessation of administration of the therapeutic agent. In particular, a cancer may become resistant to treatment with a RAS(OFF) inhibitor by the acquisition of a mutation (e.g., in the RAS protein) that decreases the efficacy of the RAS(OFF) inhibitor. Measurement of a decrease in the efficacy of the treatment will depend on the disorder being treated, and such methods are known to those of skill in the art. For example, efficacy of a cancer treatment may be measured by the progression of the disease. An effective treatment may slow or halt the progression of the disease. A cancer that is resistant to treatment with a therapeutic agent, e.g., a RAS(OFF) inhibitor, may fail to slow or halt the progression of the disease.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

A “therapeutic agent” is any substance, e.g., a compound or composition, capable of treating a disease or disorder. In some embodiments, therapeutic agents that are useful in connection with the present disclosure include RAS inhibitors and cancer chemotherapeutics. Many such therapeutic agents are known in the art and are disclosed herein.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

A “therapeutic regimen” refers to a dosing regimen whose administration across a relevant population is correlated with a desired or beneficial therapeutic outcome.

The term “thiocarbonyl,” as used herein, refers to a —C(S)— group. The term “treatment” (also “treat” or “treating”), in its broadest sense, refers to any administration of a substance (e.g., a compound of the present disclosure) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

The term “ynone,” as used herein, refers to a group comprising the structure

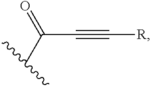

wherein R is any any chemically feasible substituent described herein.

### RAS Inhibitors

Provided herein are compounds that inhibit RAS and uses thereof. Also provided are pharmaceutical compositions including one or more RAS inhibitor compounds, or a pharmaceutically acceptable salt thereof, and a pharmaceutically acceptable excipient. RAS inhibitor compounds may be used in methods of inhibiting RAS (e.g., in a subject or in a cell) and methods of treating cancer, as described herein. In some embodiments, a compound of the present disclosure is or acts as a prodrug, such as with respect to administration to a cell or to a subject in need thereof.

### RAS(ON) Inhibitors

Provided herein are RAS(ON) inhibitors. A RAS(ON) inhibitor targets, that is, selectively binds to or inhibits the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS).

In some embodiments, the RAS(ON) inhibitor is selective for RAS that includes an amino acid substitution at G12, G13, Q61, or a combination thereof. In some embodiments, the RAS(ON) inhibitor is selective for RAS that includes an amino acid substitution selected from G12C, G12D, G12V, G13C, G13D, Q61 L, or a combination thereof. In some embodiments, the RAS(ON) inhibitor is selective for RAS that includes a G12C amino acid substitution.

In some embodiments, the RAS(ON) inhibitor is a KRAS(ON) inhibitor, where a KRAS(ON) inhibitor refers to an inhibitor that targets, that is, selectively binds to or inhibits the GTP-bound, active state of KRAS (e.g., selective over the GDP-bound, inactive state of KRAS). In some embodiments, the KRAS(ON) inhibitor is selective for KRAS that includes an amino acid substitution at G12, G13, Q61, A146, K117, L19, Q22, V14, A59, or a combination thereof. In some embodiments, the KRAS(ON) inhibitor is selective for KRAS that includes an amino acid substitution selected from G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, G13V, or a combination thereof.

In some embodiments, the RAS(ON) inhibitor is an NRAS(ON) inhibitor, where an NRAS(ON) inhibitor refers to an inhibitor that targets, that is, selectively binds to or inhibits the GTP-bound, active state of NRAS (e.g., selective over the GDP-bound, inactive state of NRAS). In some embodiments, the NRAS(ON) inhibitor is selective for NRAS that includes an amino acid substitution at G12, G13, Q61, P185, A146, G60, A59, E132, E49, T50, or a combination thereof. In some embodiments, the NRAS(ON) inhibitor is selective for NRAS that includes an amino acid substitution selected from Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, A59T, or a combination thereof.

In some embodiments, the RAS(ON) inhibitor is an HRAS(ON) inhibitor, where an HRAS(ON) inhibitor refers to an inhibitor that targets, that is selectively binds to or inhibits the GTP-bound, active state of HRAS (e.g., selective over the GDP-bound, inactive state of HRAS). In some embodiments, the HRAS(ON) inhibitor is selective for HRAS that includes an amino acid substitution at G12, G13, Q61, K117, A59, A18, D119, A66, A146, or a combination thereof. In some embodiments, the HRAS(ON) inhibitor is selective for NRAS that includes an amino acid substitution selected from Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, G12R, or a combination thereof.

In some embodiments, the RAS(ON) inhibitor is a RAS(ON)^{MULTI} inhibitor.

In some embodiments, the RAS(ON) inhibitor is a compound, or pharmaceutically acceptable salt thereof, having the structure of Formula A00:

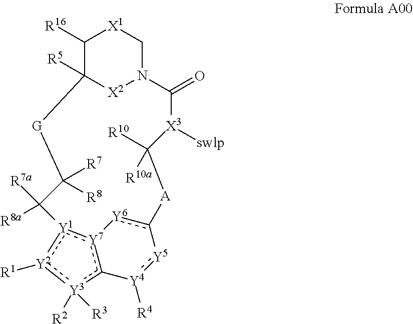

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
swlp (Switch I/P-loop) refers to an organic moiety that non-covalently binds to both the Switch I binding pocket and residues 12 or 13 of the P-loop of a Ras protein (see, e.g., Johnson et al., 292:12981-12993 (2017), incorporated herein by reference);
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R¹⁰ is hydrogen, halo, hydroxy, C₁-C₃ alkoxy, or C₁-C₃ alkyl;
R^{10a} is hydrogen or halo; and
R¹⁶ is hydrogen or C₁-C₃ alkyl (e.g., methyl). In some embodiments, the resulting compound is capable of achieving an IC50 of 2 uM or less (e.g., 1.5 uM, 1 uM, 500 nM, or 100 nM or less) in the Ras-RAF disruption assay protocol described herein.

In some embodiments, the disclosure features a compound, or pharmaceutically acceptable salt thereof, of structural Formula AI:

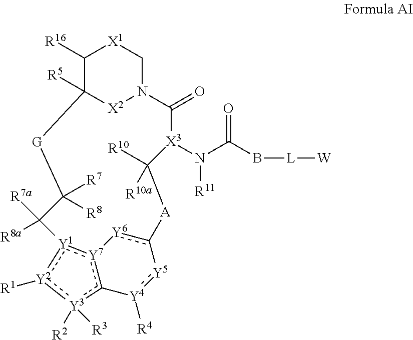

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl (e.g., methyl).

In some embodiments, the disclosure features a compound, or pharmaceutically acceptable salt thereof, of structural Formula Ala:

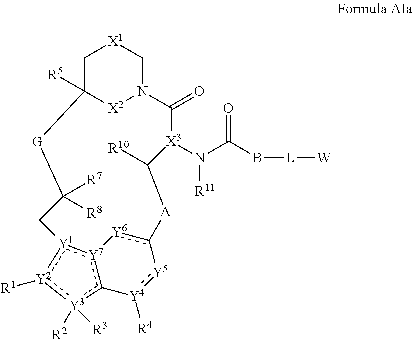

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the disclosure features a compound, or pharmaceutically acceptable salt thereof, of structural Formula Alb:

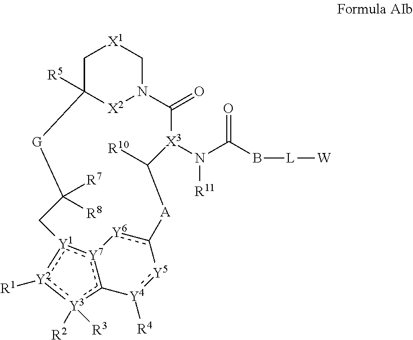

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula A1 and subformula thereof, G is optionally substituted C₁-C₄ heteroalkylene.

In some embodiments, the RAS(ON) inhibitor has the structure of Formula AIc, or a pharmaceutically acceptable salt thereof:

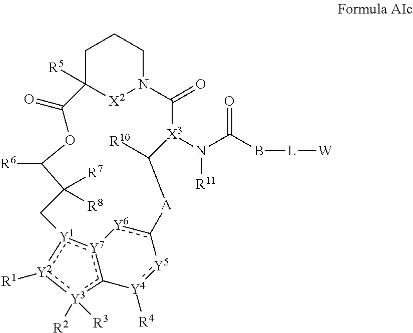

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula AI and subformula thereof, X² is NH. In some embodiments of Formula AI and subformula thereof, X³ is CH.

In some embodiments of Formula AI and subformula thereof, R¹¹ is hydrogen. In some embodiments of Formula AI and subformula thereof, R¹¹ is C₁-C₃ alkyl. In some embodiments of Formula AI and subformula thereof, R¹¹ is methyl.

In some embodiments, the RAS(ON) inhibitor has the structure of Formula AId, or a pharmaceutically acceptable salt thereof:

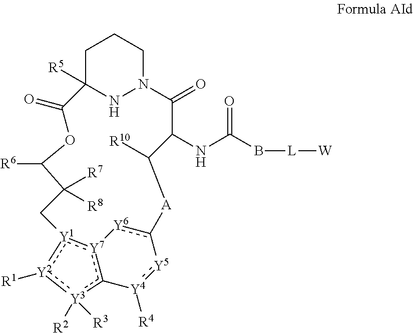

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of compounds of the present invention, X¹ is optionally substituted C₁-C₂ alkylene. In some embodiments, X¹ is methylene. In some embodiments, X¹ is methylene substituted with a C₁-C₆ alkyl group or a halogen. In some embodiments, X¹ is —CH(Br)—. In some embodiments, X¹ is —CH(CH₃)—.

In some embodiments of Formula AI and subformula thereof, R³ is absent.

In some embodiments of Formula AI and subformula thereof, R⁴ is hydrogen.

In some embodiments of Formula AI and subformula thereof, R⁵ is hydrogen. In some embodiments of Formula AI and subformula thereof, R⁵ is C₁-C₄ alkyl optionally substituted with halogen.

In some embodiments of Formula AI and subformula thereof, R⁵ is methyl.

In some embodiments of Formula AI and subformula thereof, Y⁴ is C. In some embodiments of Formula AI and subformula thereof, Y⁵ is CH. In some embodiments of Formula AI and subformula thereof, Y⁶ is CH. In some embodiments of Formula AI and subformula thereof, Y¹ is C. In some embodiments of Formula AI and subformula thereof, Y² is C. In some embodiments of Formula AI and subformula thereof, Y³ is N. In some embodiments of Formula AI and subformula thereof, Y⁷ is C.

In some embodiments, the RAS(ON) inhibitor has the structure of Formula Ale, or a pharmaceutically acceptable salt thereof:

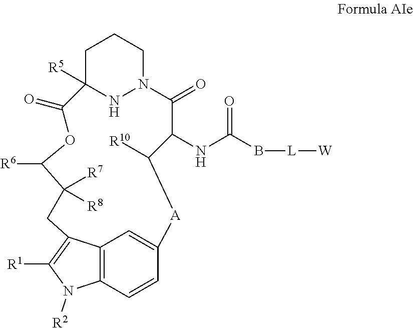

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula AI and subformula thereof, R⁶ is hydrogen.

In some embodiments of Formula AI and subformula thereof, R² is hydrogen, cyano, optionally substituted C₁-C₆ alkyl, optionally substituted 3 to 6-membered cycloalkyl, or optionally substituted 3 to 6-membered heterocycloalkyl. In some embodiments of Formula AI and subformula thereof, R² is optionally substituted C₁-C₆ alkyl, such as ethyl. In some embodiments of Formula AI and subformula thereof, R² is fluoro C₁-C₆ alkyl, such as —CH₂CH₂F, —CH₂CHF₂, or —CH₂CF₃.

In some embodiments of Formula AI and subformula thereof, R⁷ is optionally substituted C₁-C₃ alkyl. In some embodiments of Formula AI and subformula thereof, R⁷ is C₁-C₃ alkyl.

In some embodiments of Formula AI and subformula thereof, R⁸ is optionally substituted C₁-C₃ alkyl. In some embodiments of Formula AI and subformula thereof, R⁸ is C₁-C₃ alkyl, such as methyl.

In some embodiments, the RAS(ON) inhibitor has the structure of Formula Alf, or a pharmaceutically acceptable salt thereof:

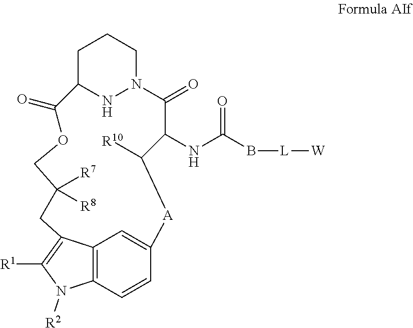

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, R¹ is 5 to 10-membered heteroaryl.

In some embodiments, R¹ is optionally substituted 6-membered aryl or optionally substituted 6-membered heteroaryl.

In some embodiments of Formula AI and subformula thereof, R₁ is

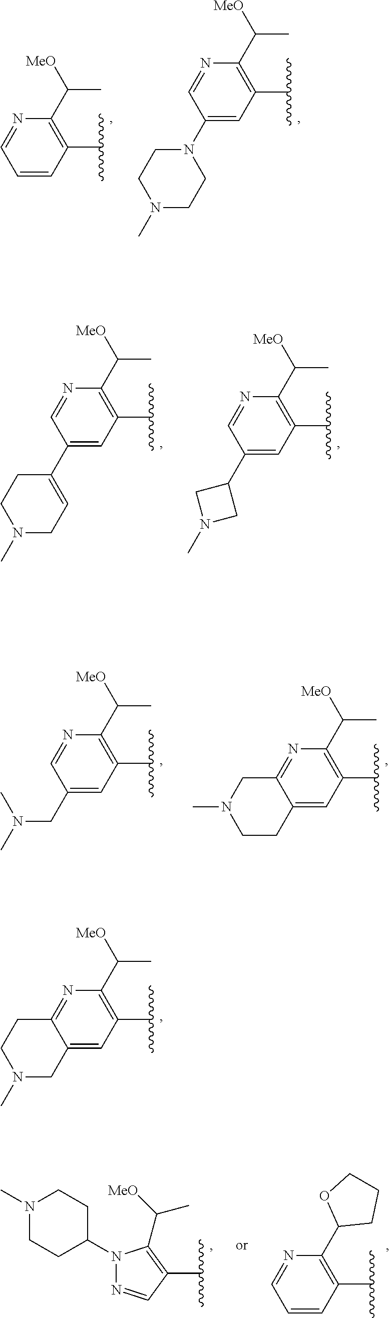

or a stereoisomer thereof. In some embodiments, R₁ is

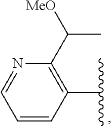 [SMILES: *c1cccnc1C(C)OC]

or a stereoisomer thereof. In some embodiments, R₁ is

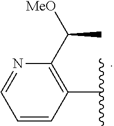 [SMILES: *c1cccnc1[C@H](C)OC]

In some embodiments, R₁ is

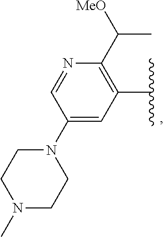 [SMILES: *c1cc(N2CCN(C)CC2)cnc1C(C)OC]

or a stereoisomer thereof. In some embodiments, R₁ is

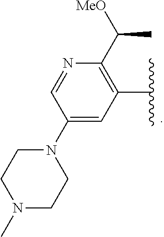 [SMILES: *c1cc(N2CCN(C)CC2)cnc1[C@H](C)OC]

In some embodiments, the RAS(ON) inhibitor has the structure of Formula Alg, or a pharmaceutically acceptable salt thereof:

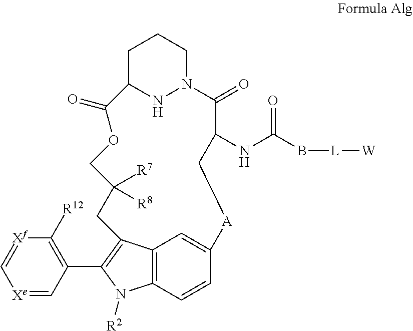

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is N, CH, or CR¹⁷;
X^{f} is N or CH;
R¹² is optionally substituted C₁-C₆ alkyl or optionally substituted C₁-C₆ heteroalkyl; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments of Formula AI and subformula thereof, X^{e} is N and X^{f} is CH. In some embodiments, X^{e} is CH and X^{f} is N. In some embodiments, X^{e} is CR¹⁷ and X^{f} is N.

In some embodiments of Formula AI and subformula thereof, R¹² is optionally substituted C₁-C₆ heteroalkyl. In some embodiments, R¹² is

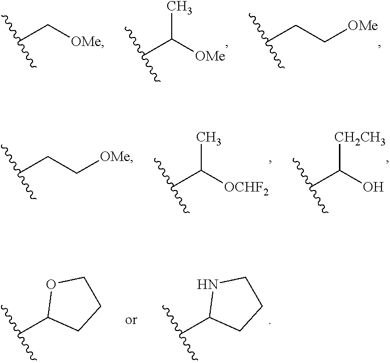

In some embodiments, the RAS(ON) inhibitor has the structure of Formula AIh, or a pharmaceutically acceptable salt thereof:

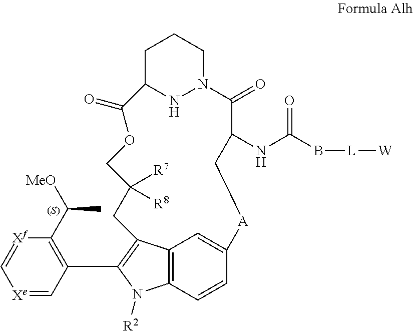

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is CH, or CR¹⁷; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS(ON) inhibitor has the structure of Formula Ali, or a pharmaceutically acceptable salt thereof:

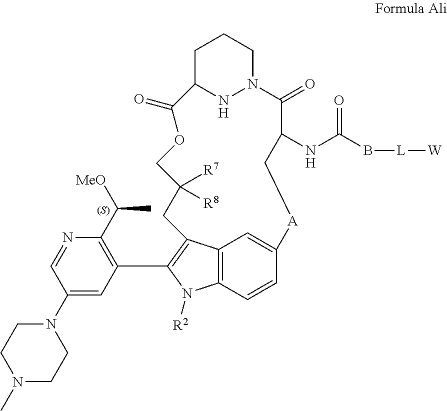

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, A is optionally substituted 6-membered arylene. In some embodiments, A has the structure:

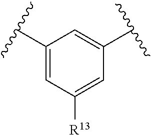

wherein R¹³ is hydrogen, hydroxy, amino, cyano, optionally substituted C₁-C₆ alkyl, or optionally substituted C₁-C₆ heteroalkyl. In some embodiments, R¹³ is hydrogen. In some embodiments, R¹³ is hydroxy. In some embodiments, A is an optionally substituted 5 to 10-membered heteroarylene. In some embodiments, A is:

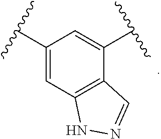 [SMILES: *c1cc(*)c2cn[nH]c2c1]

In some embodiments, A is optionally substituted 5 to 6-membered heteroarylene. In some embodiments, A is:

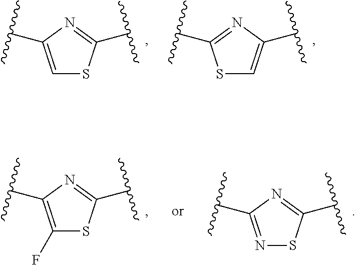 [SMILES: *c1csc(*)n1 ; *c1csc(*)n1 ; *c1nc(*)c(F)s1 ; *c1nsc(*)n1]

In some embodiments, A is

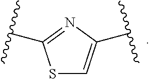 [SMILES: *c1csc(*)n1]

In some embodiments of Formula AI and subformula thereof, B is —CHR⁹—. In some embodiments, R⁹ is optionally substituted C₁-C₆ alkyl or optionally substituted 3 to 6-membered cycloalkyl. In some embodiments, R⁹ is:

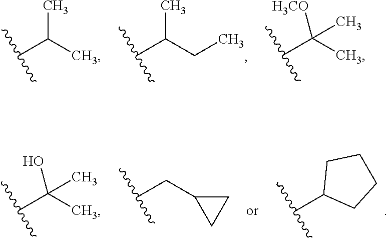

In some embodiments, R⁹ is:

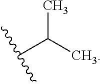 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, B is optionally substituted 6-membered arylene.

In some embodiments, B is 6-membered arylene. In some embodiments, B is:

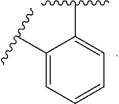 [SMILES: *c1ccccc1*]

In some embodiments B is absent.

In some embodiments of Formula AI and subformula thereof, R⁷ is methyl.

In some embodiments of Formula AI and subformula thereof, R⁸ is methyl.

In some embodiments of Formula AI and subformula thereof, R¹⁶ is hydrogen.

In some embodiments of Formula AI and subformula thereof, the linker is the structure of Formula AII:

A¹-(B¹)_{f}—(C¹)_{g}—(B²)ₕ-(D¹)-(B³)ᵢ—(C²)ⱼ—(B⁴)ₖ-A² Formula AII
where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₁-C₃ cycloalkyl, optionally substituted C_{2-C4} alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments, the linker is acyclic. In some embodiments, the linker has the structure of Formula AIIa:

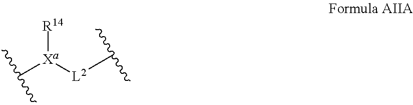

wherein X^{a} is absent or N;
R¹⁴ is absent, hydrogen or optionally substituted C₁-C₆ alkyl or optionally substituted C₁-C₃ cycloalkyl; and
L² is absent, —C(O)—, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

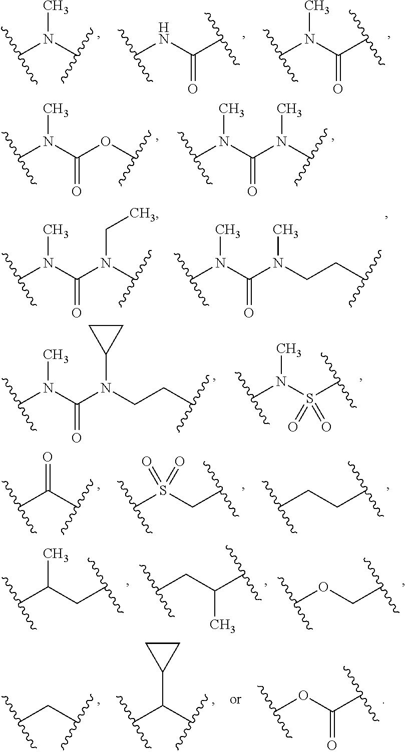

In some embodiments, L is

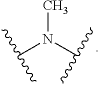 [SMILES: *N(*)C]

In some embodiments, L is

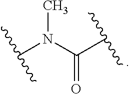 [SMILES: *C(=O)N(*)C]

In some embodiments, linker is or comprises a cyclic group. In some embodiments of Formula AI and subformula thereof, the linker has the structure of Formula AIIb:

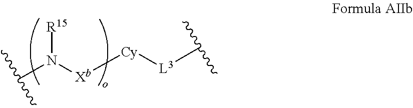

wherein o is 0 or 1;
X^{b} is C(O) or SO₂;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —C(O)—, —SO₂—, optionally substituted C₁-C₄ alkylene or optionally substituted C₁-C₄ heteroalkylene. In some embodiments, the linker has the structure:

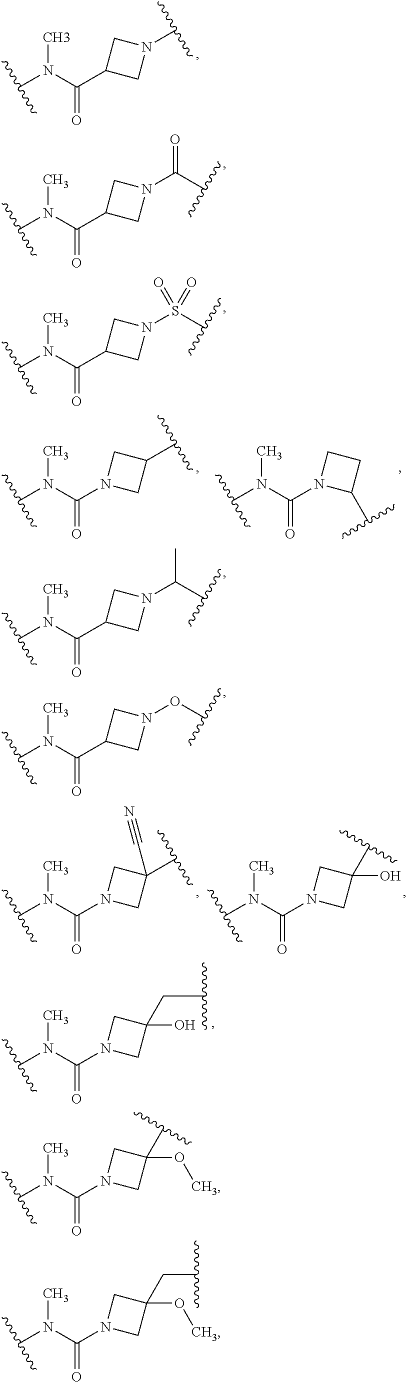

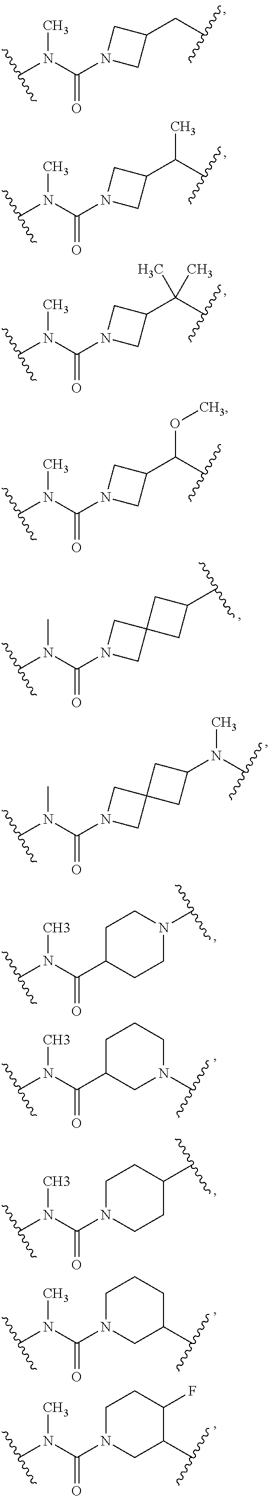

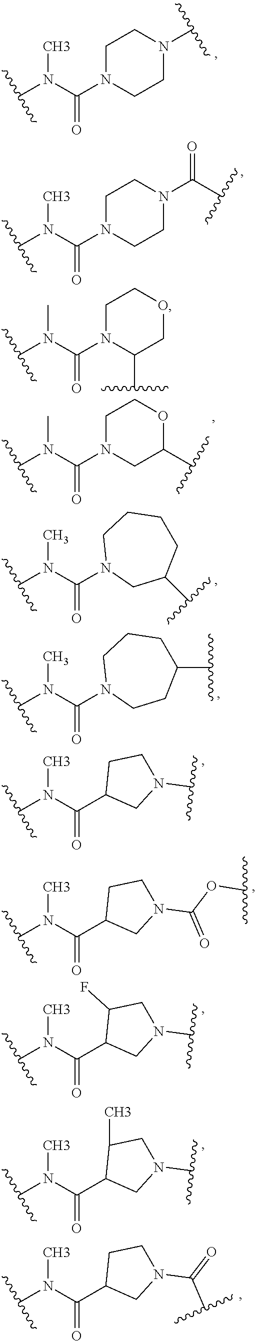

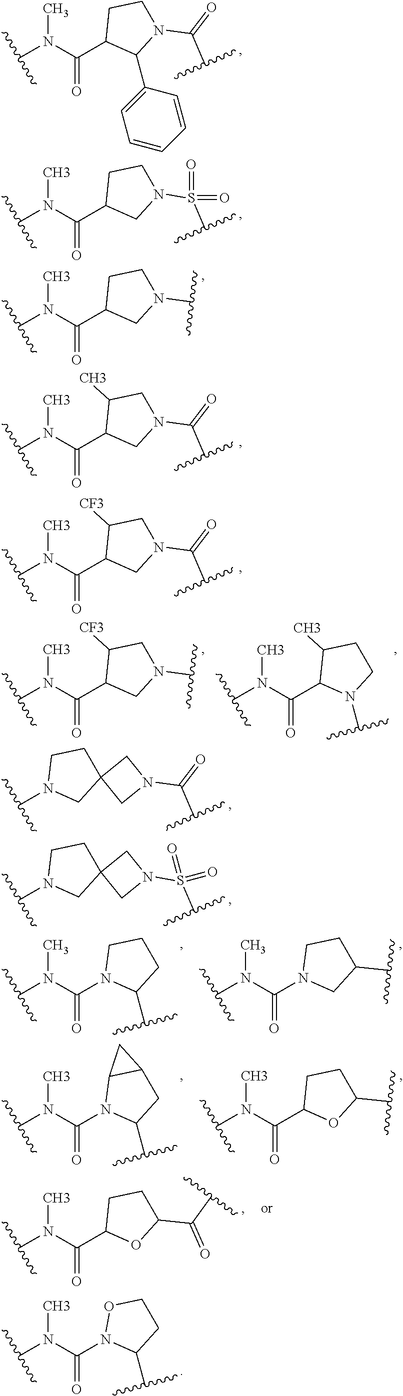

In some embodiments of Formula AI and subformula thereof, W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 8-membered heterocycloalkyl, optionally substituted 3 to 8-membered cycloalkyl, or 3 to 8-membered heteroaryl.

In some embodiments of Formula AI and subformula thereof, W is hydrogen. In some embodiments of Formula AI and subformula thereof, W is optionally substituted amino. In some embodiments of Formula AI and subformula thereof, W is —NHCH₃ or —N(CH₃)₂. In some embodiments of Formula AI and subformula thereof, W is optionally substituted C₁-C₄ alkoxy. In some embodiments, W is methoxy or iso-propoxy. In some embodiments of Formula AI and subformula thereof, W is optionally substituted C₁-C₄ alkyl. In some embodiments, W is methyl, ethyl, iso-propyl, tert-butyl, or benzyl. In some embodiments of Formula AI and subformula thereof, W is optionally substituted amido. In some embodiments, W is

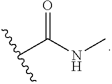 [SMILES: *C(=O)NC]

In some embodiments, W is

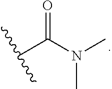 [SMILES: *C(=O)N(C)C]

In some embodiments of Formula AI and subformula thereof, W is optionally substituted C₁-C₄ hydroxyalkyl. In some embodiments, W is

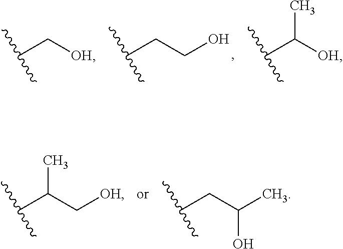

In some embodiments of Formula AI and subformula thereof, W is optionally substituted C₁-C₄ aminoalkyl. In some embodiments, W is

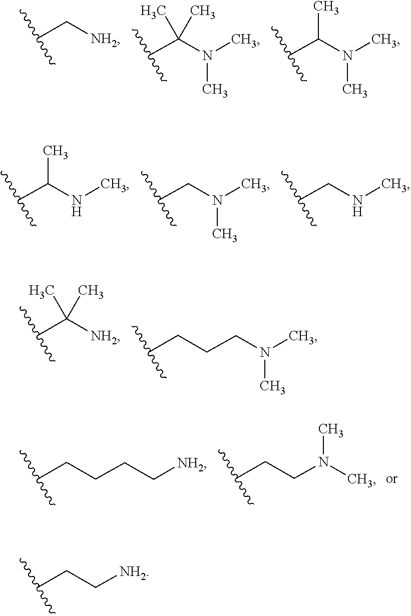

In some embodiments of Formula AI and subformula thereof, W is optionally substituted C₁-C₄ haloalkyl. In some embodiments, W is

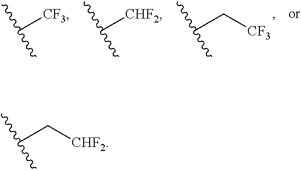

In some embodiments of Formula AI and subformula thereof, W is optionally substituted C₁-C₄ guanidinoalkyl. In some embodiments, W is

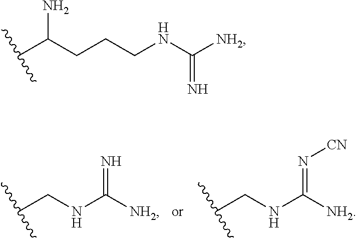 [SMILES: *C(N)CCCNC(=N)N ; *CNC(=N)N ; *CN/C(N)=N/C#N]

In some embodiments of Formula AI and subformula thereof, W is C₀-C₄ alkyl optionally substituted 3 to 11-membered heterocycloalkyl. In some embodiments, W is

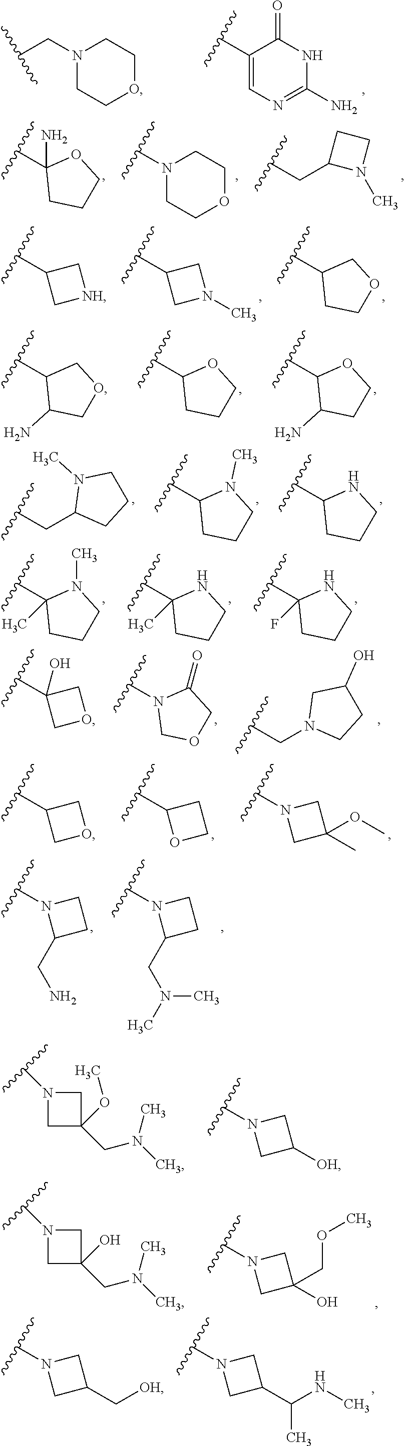

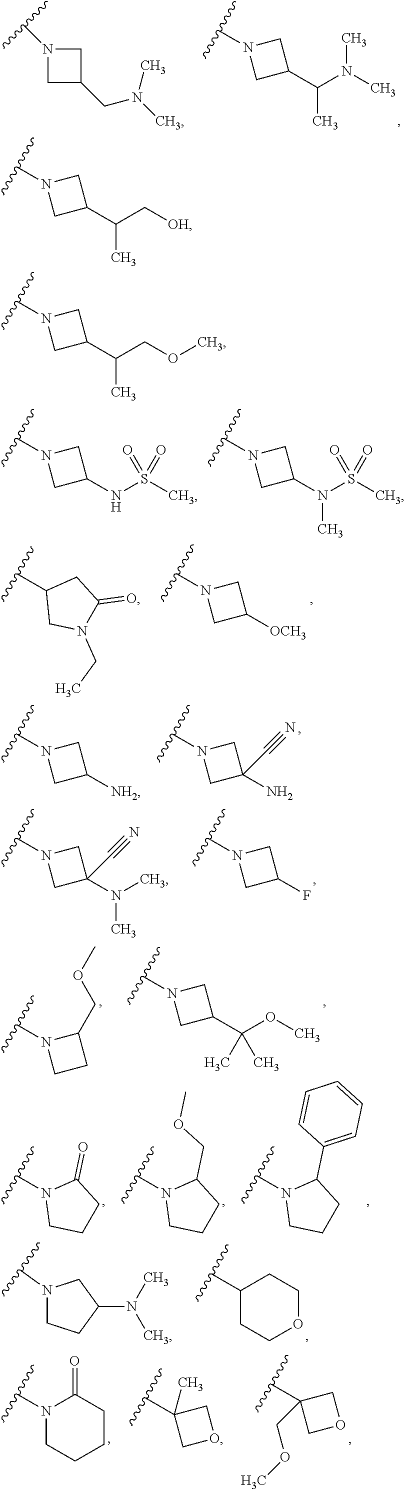

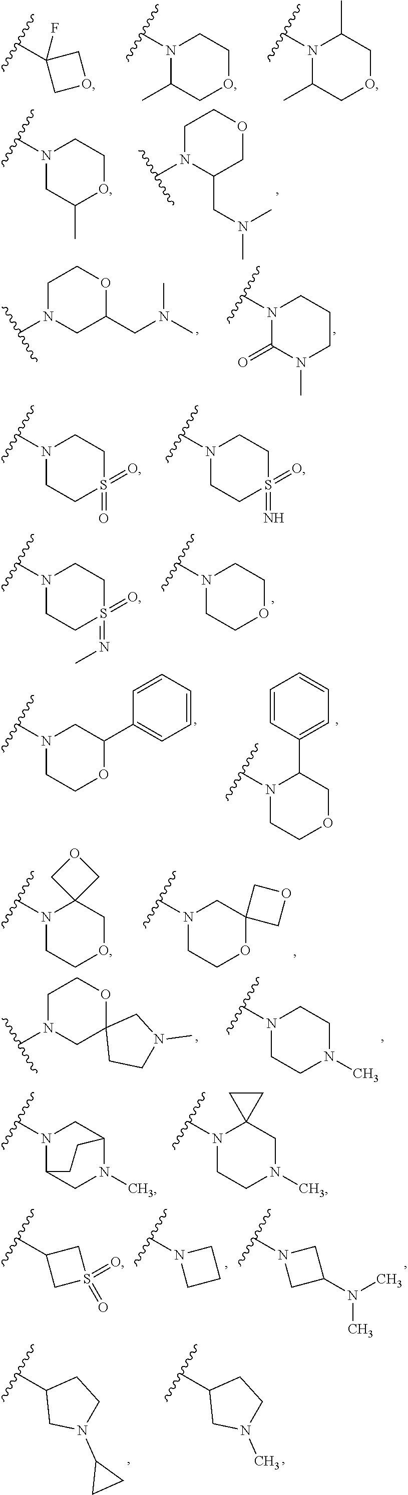

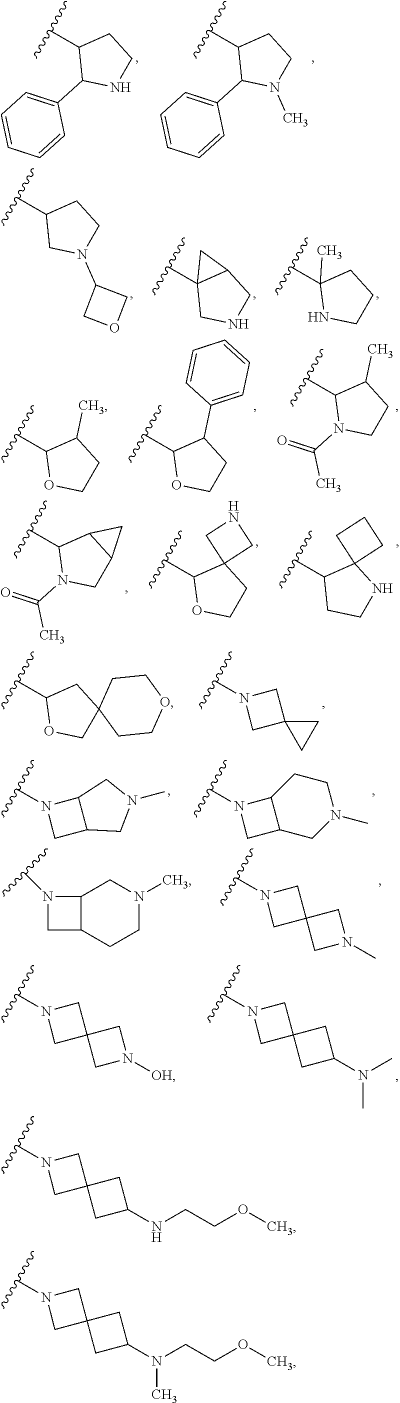

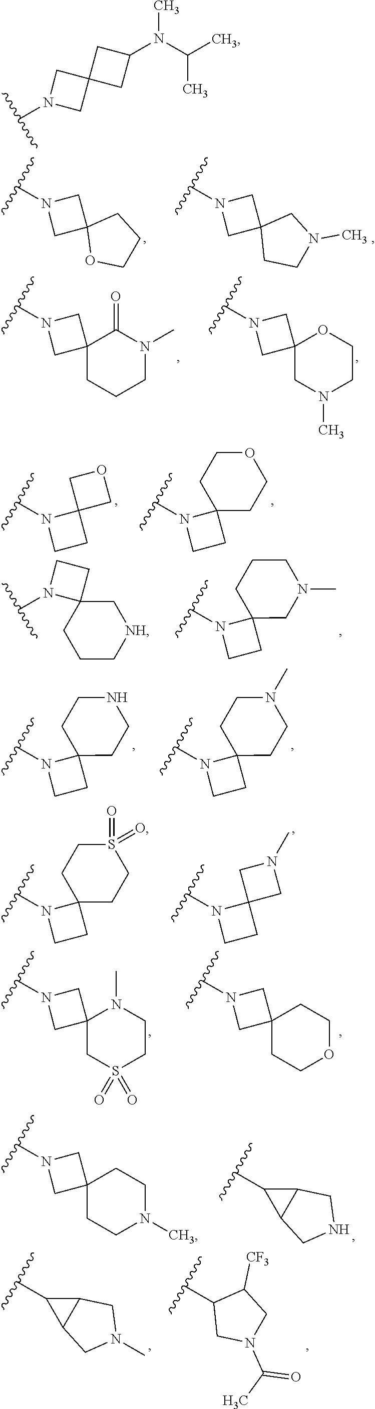

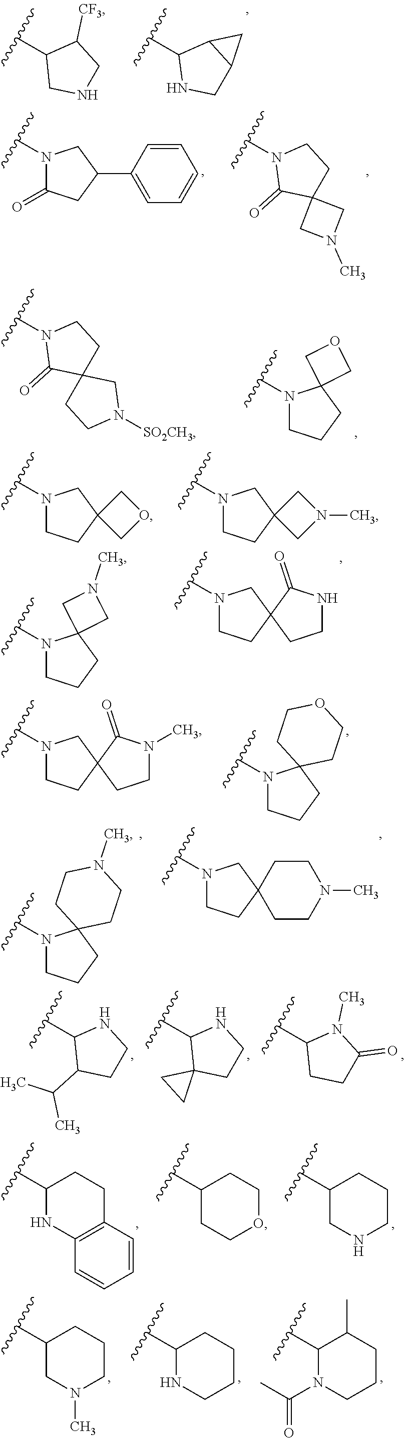

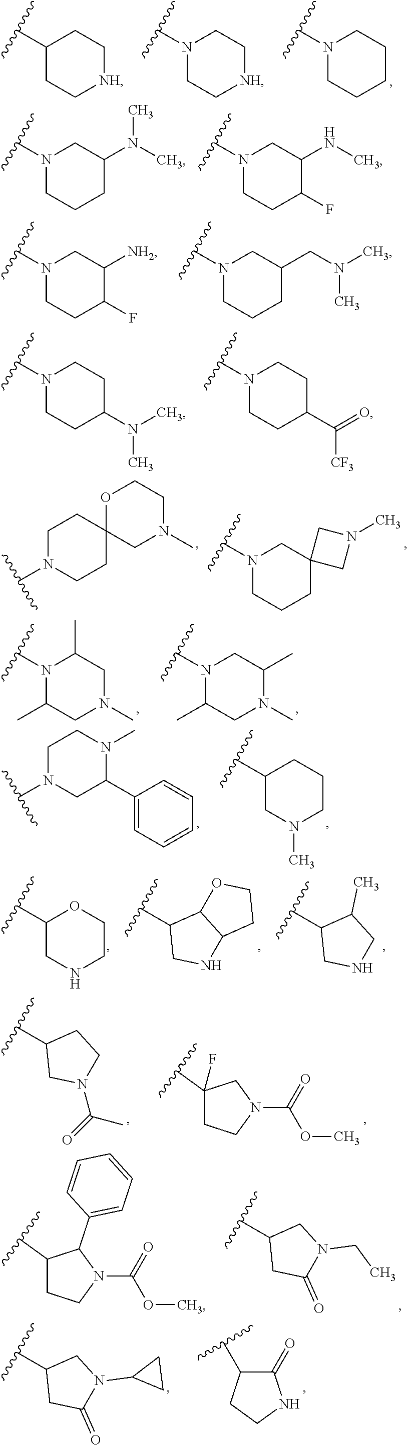

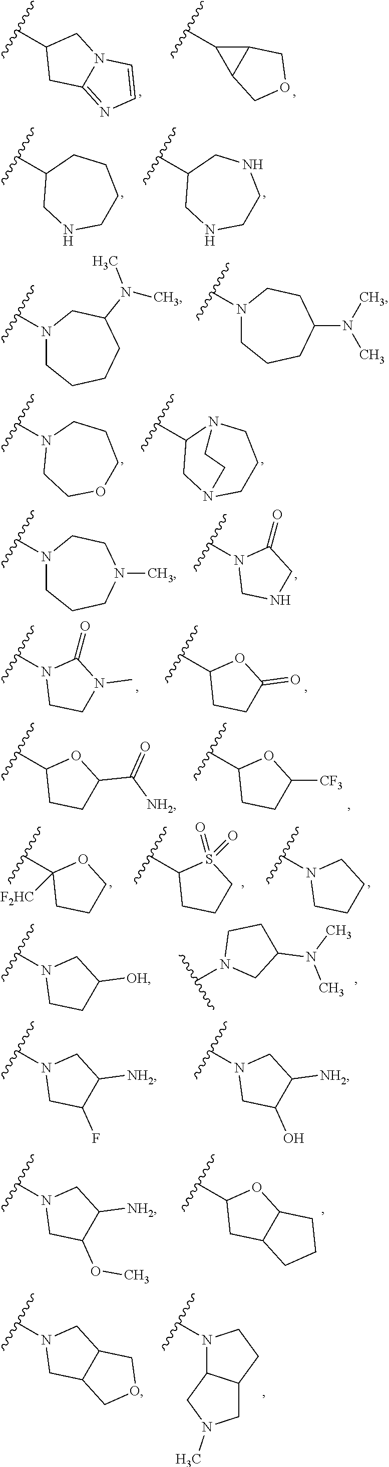

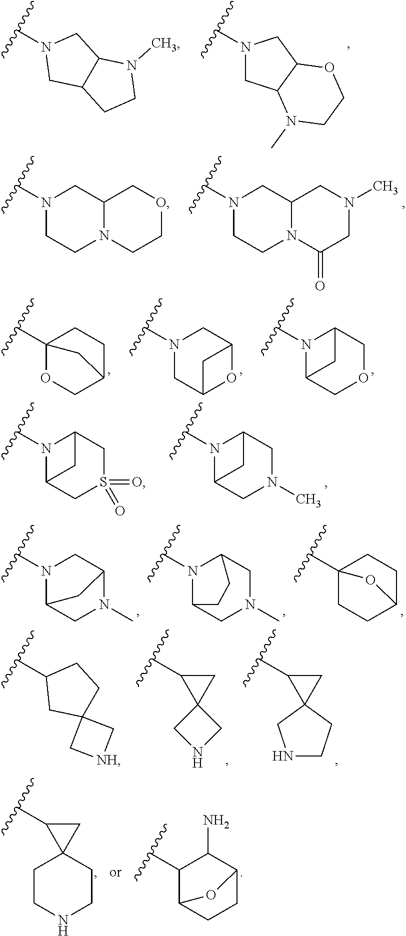

In some embodiments of Formula AI and subformula thereof, W is optionally substituted 3 to 8-membered cycloalkyl. In some embodiments, W is

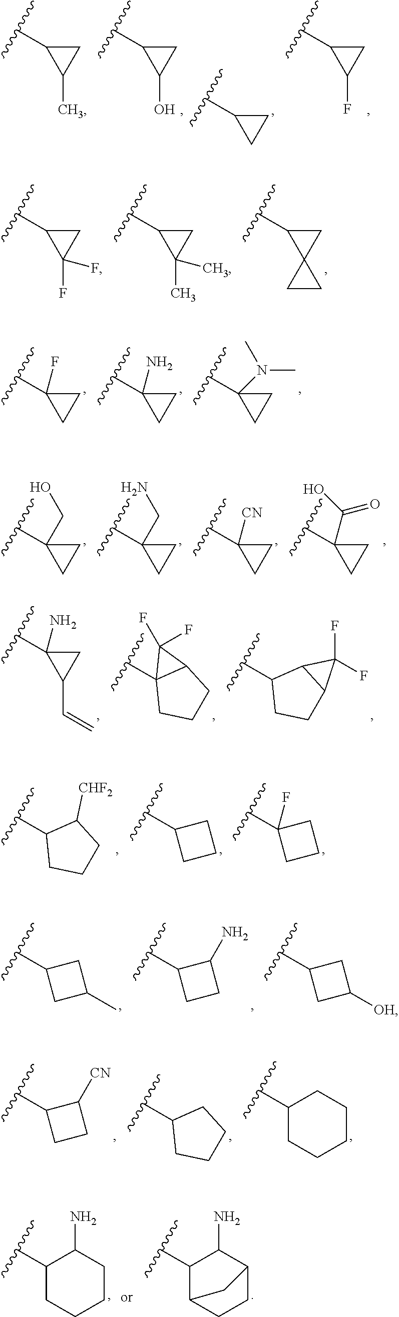

In some embodiments of Formula AI and subformula thereof, W is optionally substituted 3 to 8-membered heteroaryl. In some embodiments, W is

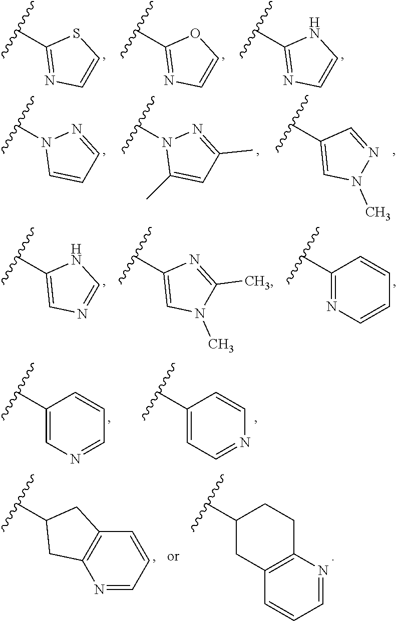

In some embodiments of Formula AI and subformula thereof, W is optionally substituted 6- to 10-membered aryl (e.g., phenyl, 4-hydroxy-phenyl, or 2,4-methoxy-phenyl).

In some embodiments, the RAS(ON) inhibitor is selected from Table A1, or a pharmaceutically acceptable salt or stereoisomer thereof. In some embodiments, the RAS(ON) inhibitor is selected from Table A1, or a pharmaceutically acceptable salt or atropisomer thereof.

TABLE A1
Certain Compounds of the Present Invention

| Ex# | Structure |
|---|---|
| AA1 | 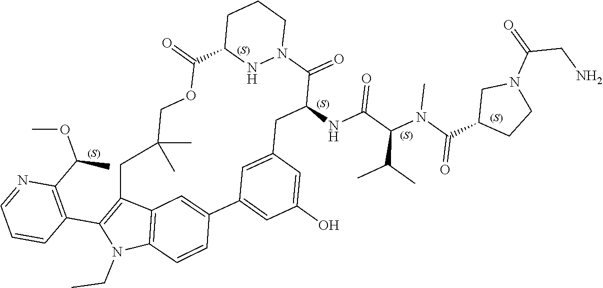 |
| AA2 | 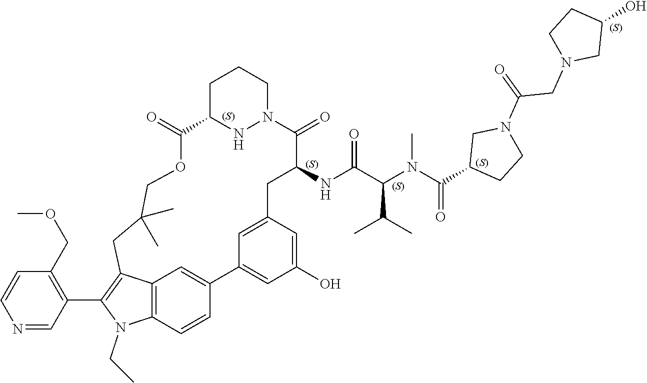 |
| AA3 | 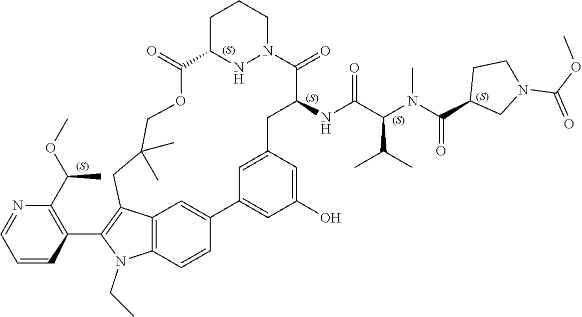 |
| AA4 | 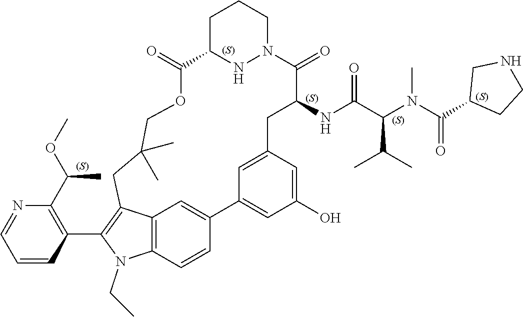 |
| AA5 | 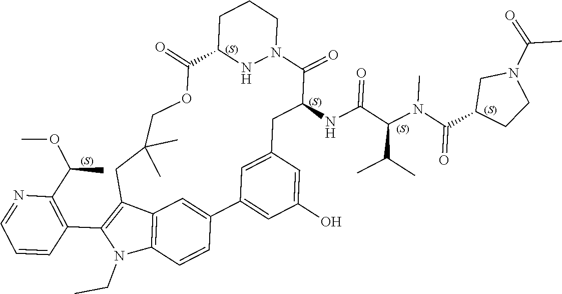 |
| AA6 | 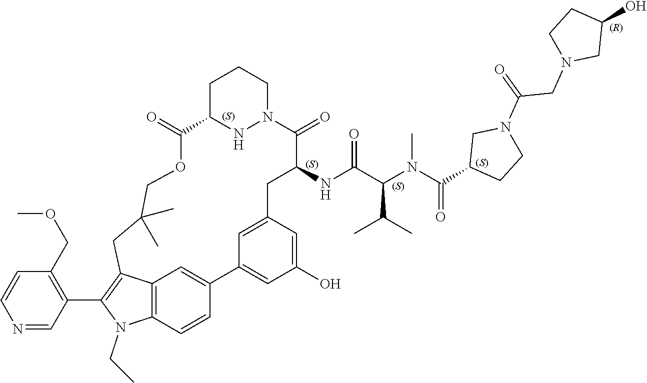 |
| AA7 | 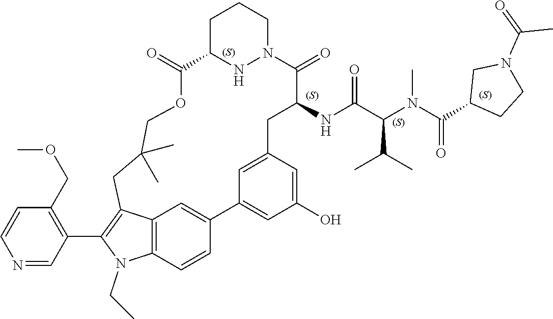 |
| AA8 | 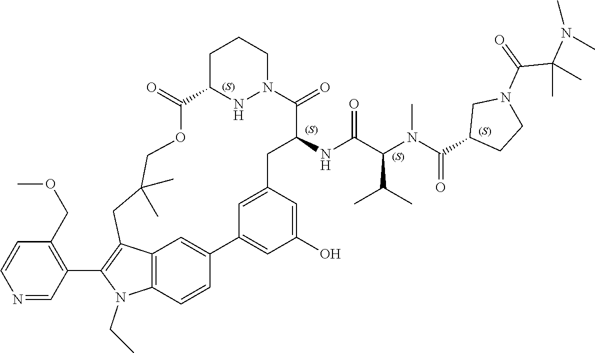 |
| AA9 | 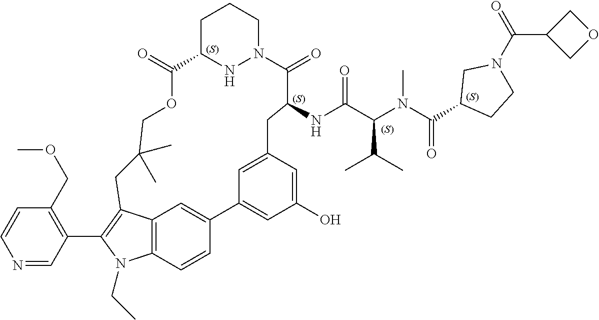 |
| AA10 | 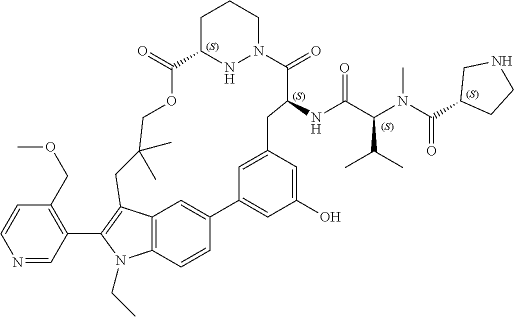 |
| AA11 | 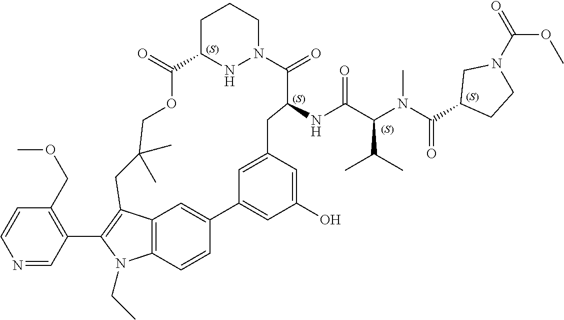 |
| AA12 | 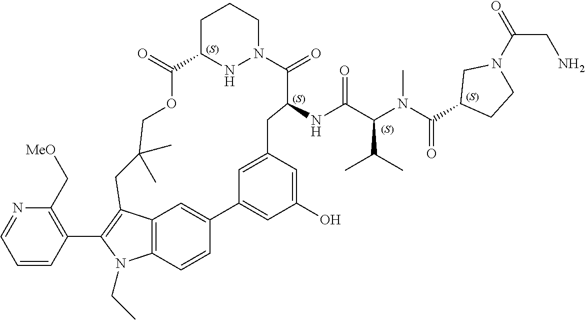 |
| AA13 | 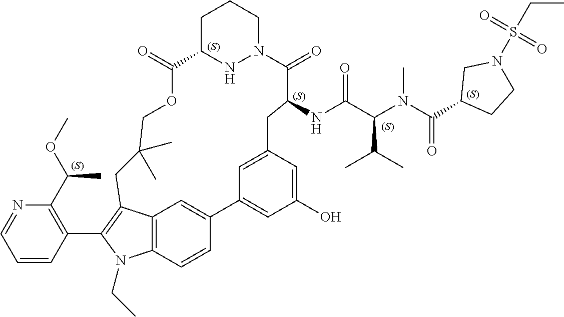 |
| AA14 | 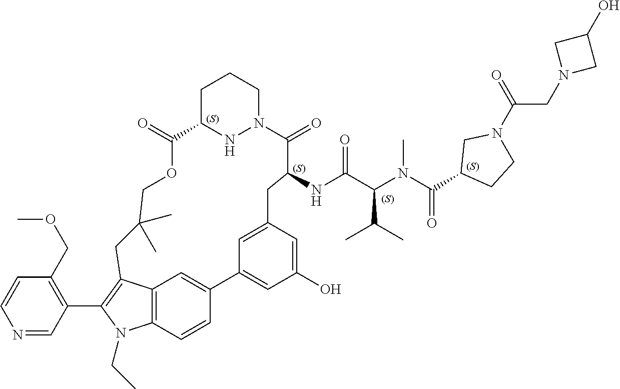 |
| AA15 | 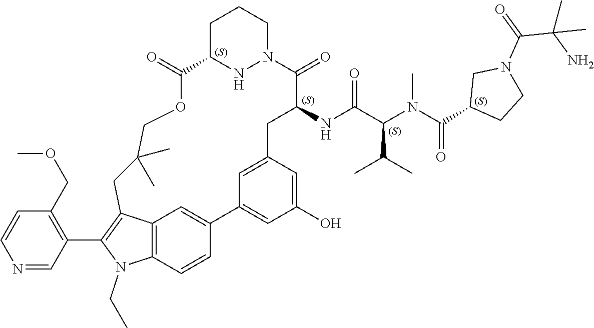 |
| AA16 | 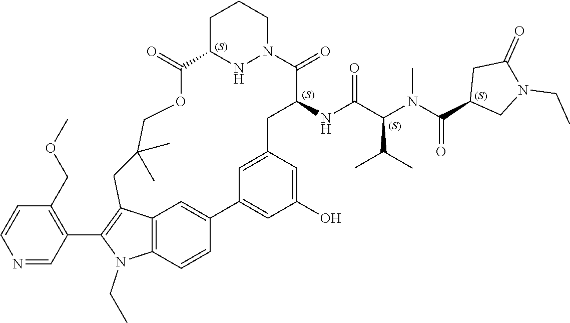 |
| AA17 | 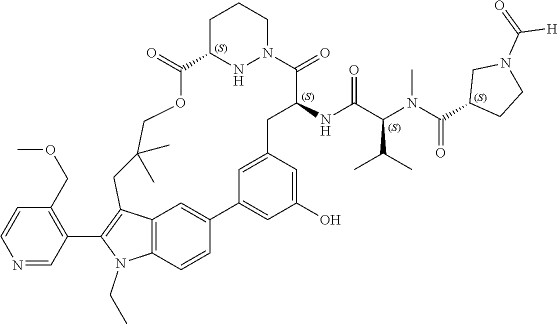 |
| AA18 | 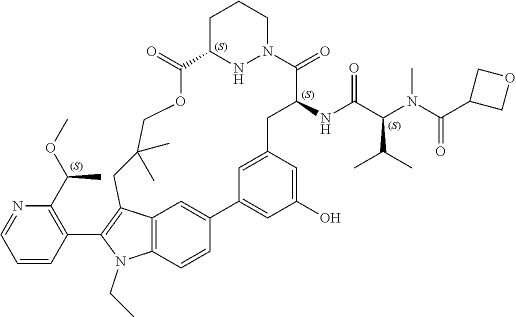 |
| AA19 | 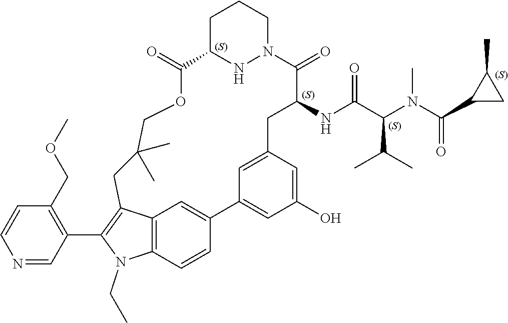 |
| AA20 | 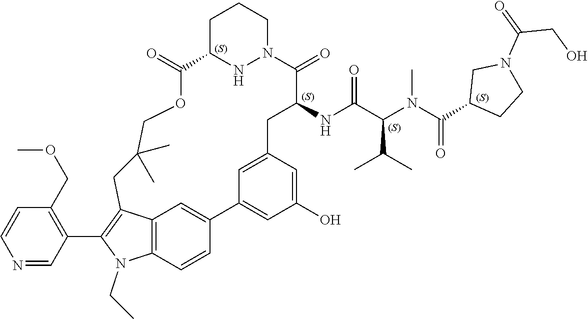 |
| AA21 | 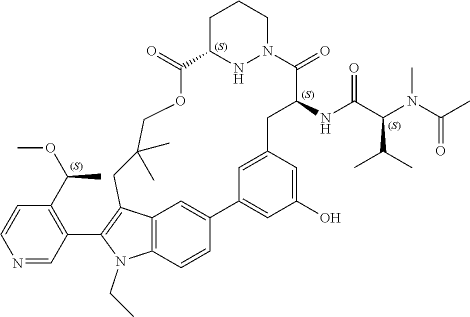 |
| AA22 | 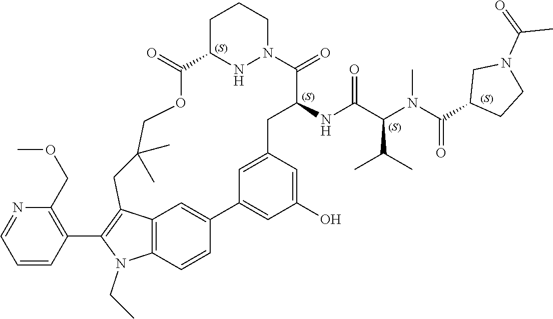 |
| AA23 | 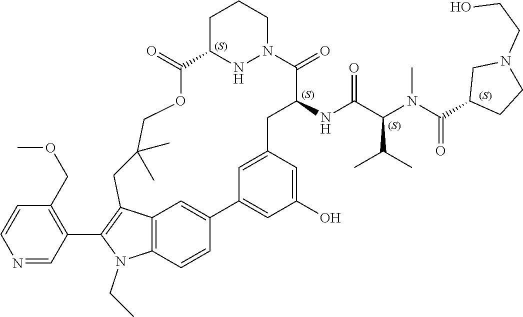 |
| AA24 | 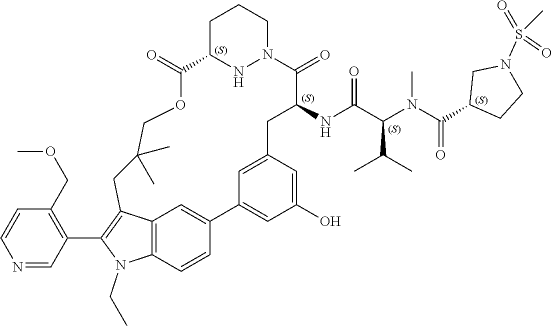 |
| AA25 | 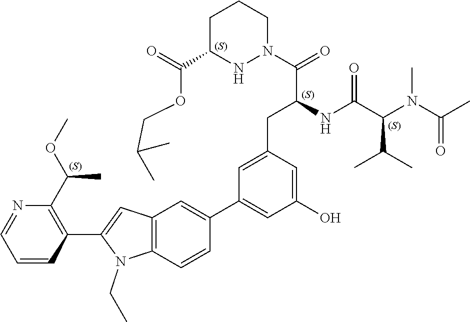 |
| AA26 | 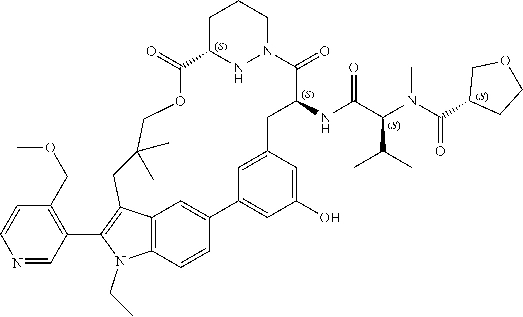 |
| AA27 | 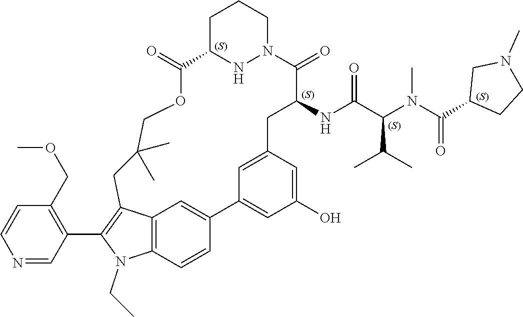 |
| AA28 | 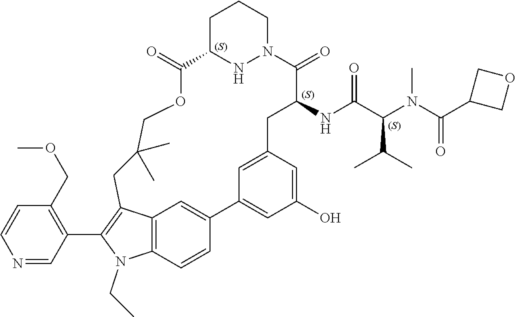 |
| AA29 | 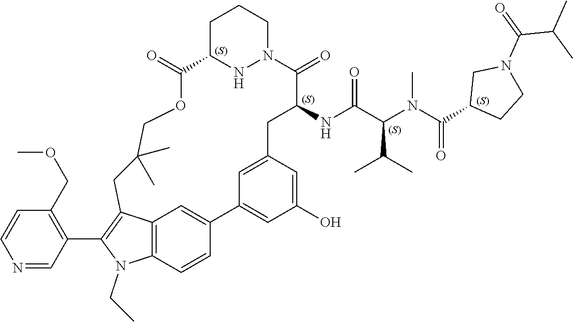 |
| AA30 | 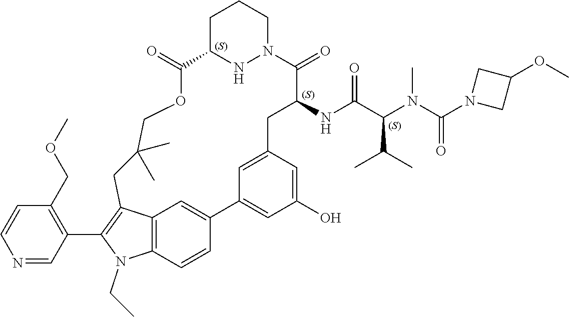 |
| AA31 | 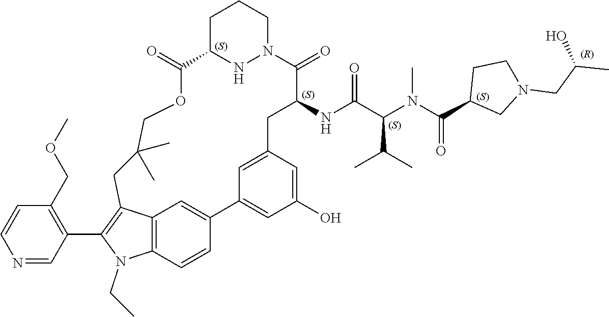 |
| AA32 | 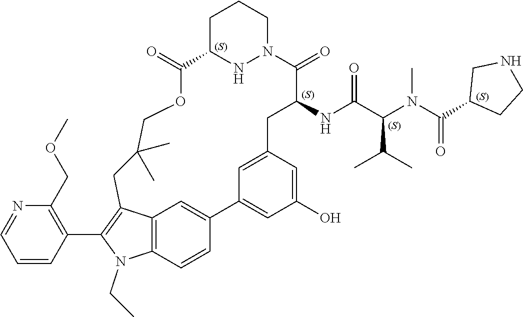 |
| AA33 | 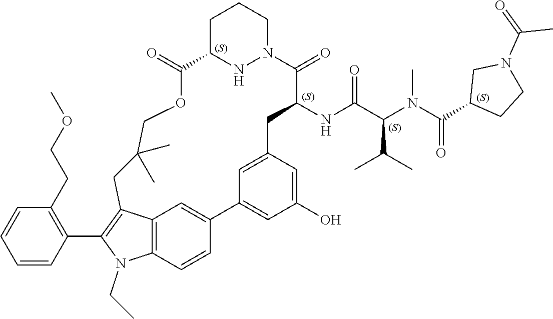 |
| AA34 | 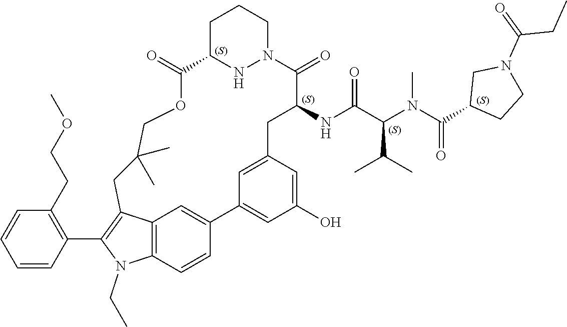 |
| AA35 | 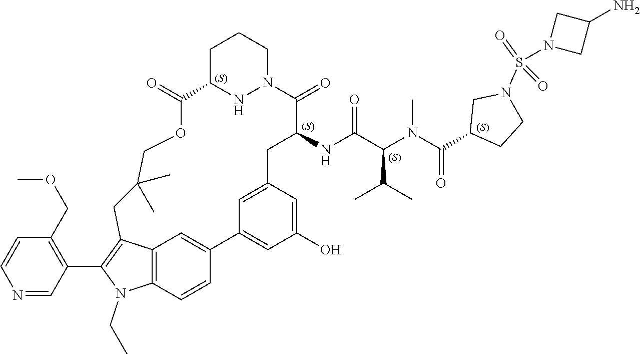 |
| AA36 | 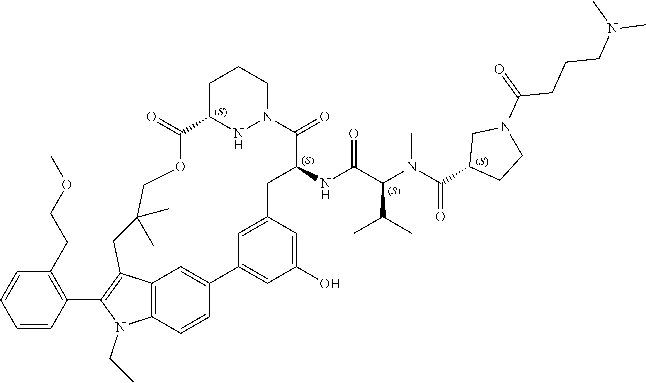 |
| AA37 | 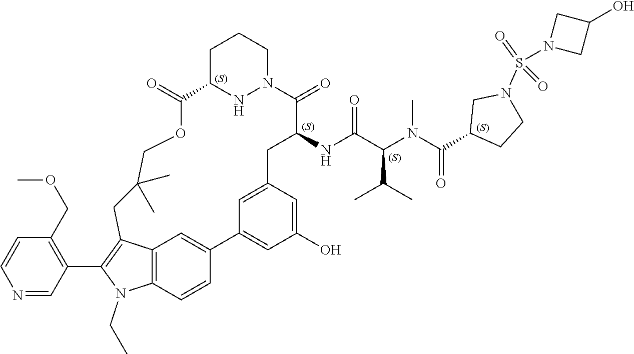 |
| AA38 | 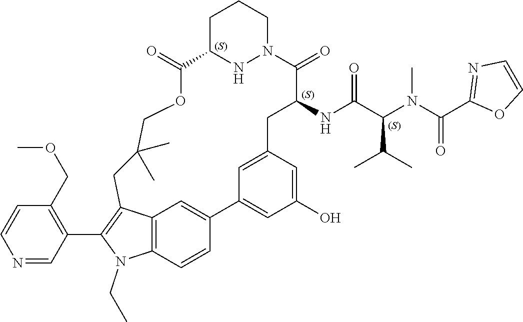 |
| AA39 | 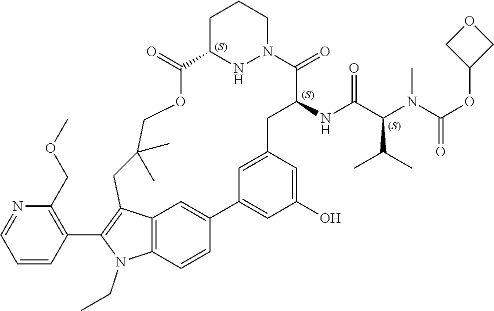 |
| AA40 | 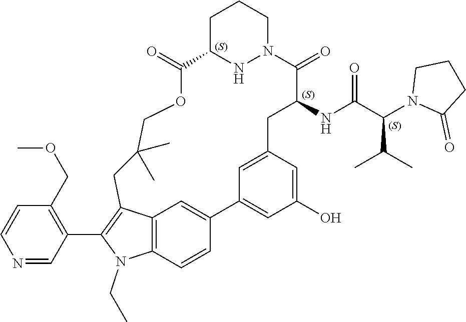 |
| AA41 | 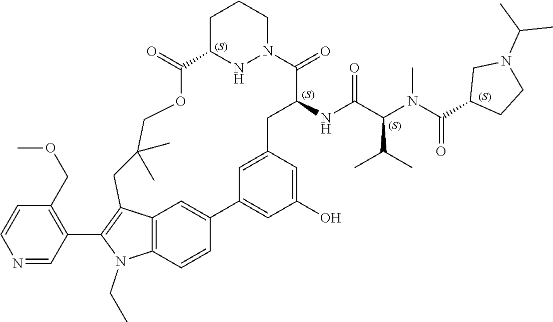 |
| AA42 | 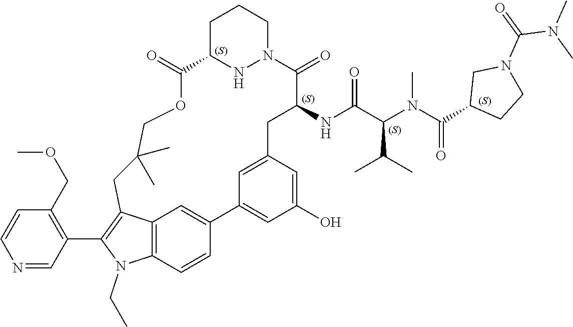 |
| AA43 | 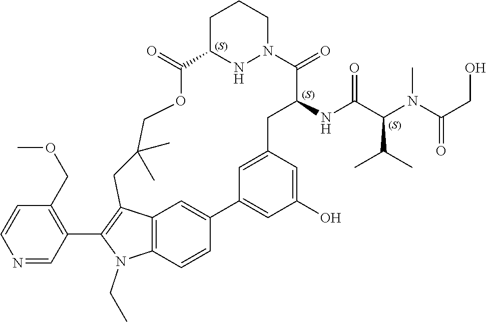 |
| AA44 | 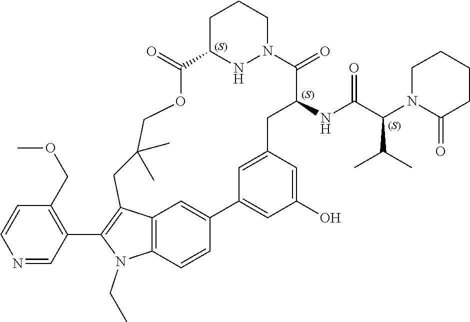 |
| AA45 | 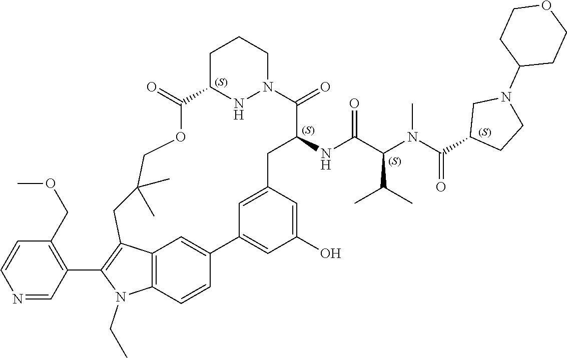 |
| AA46 | 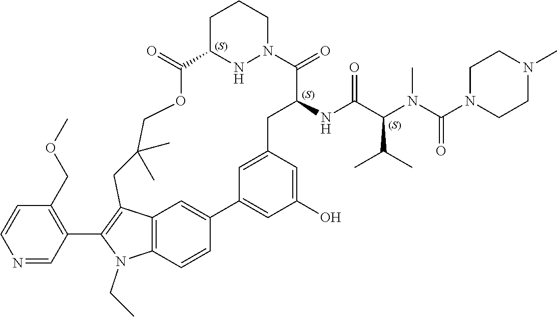 |
| AA47 | 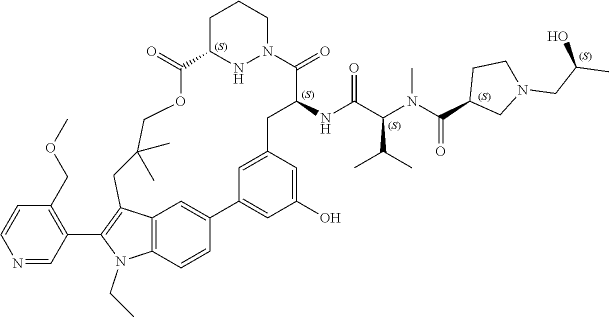 |
| AA48 | 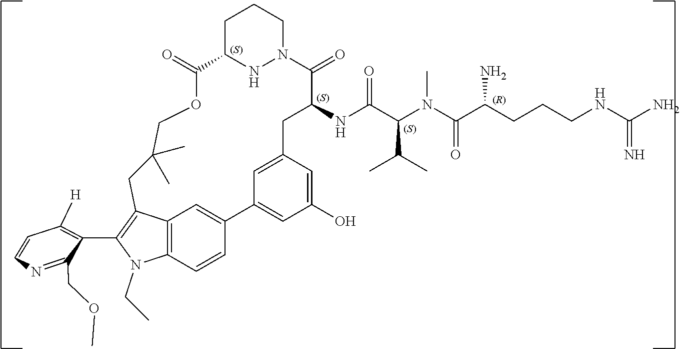 |
| AA49 | 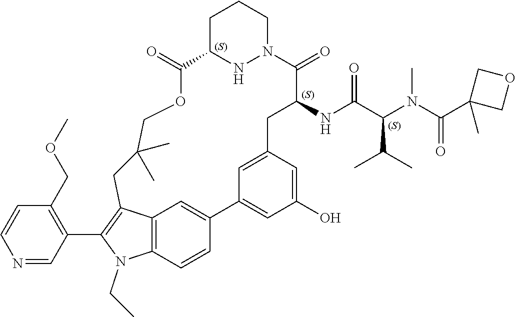 |
| AA50 | 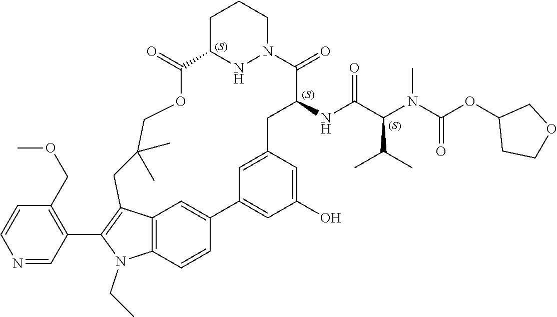 |
| AA51 | 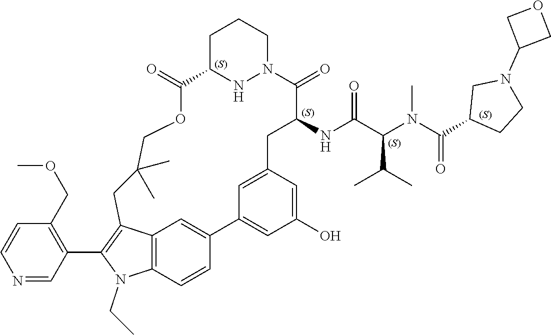 |
| AA52 | 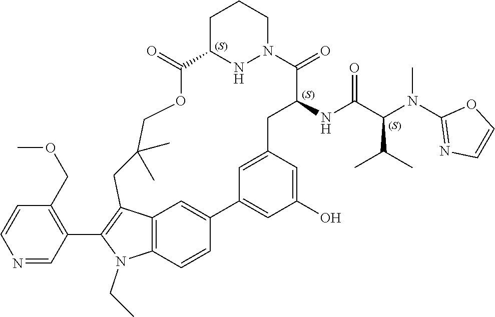 |
| AA53 | 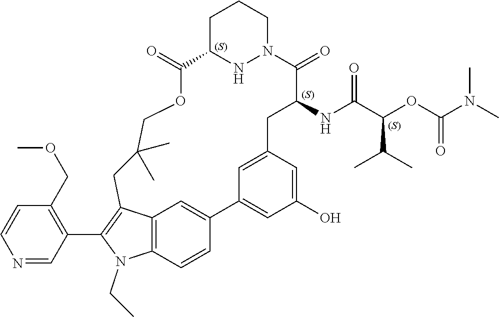 |
| AA54 | 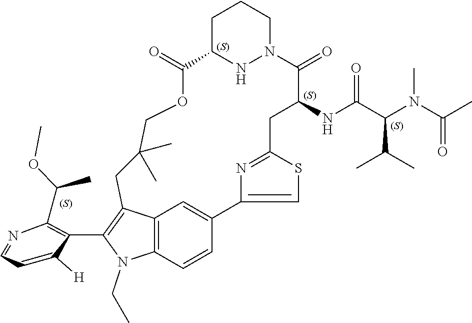 |
| AA55 | 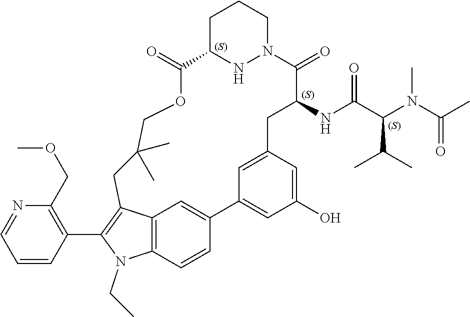 |
| AA56 | 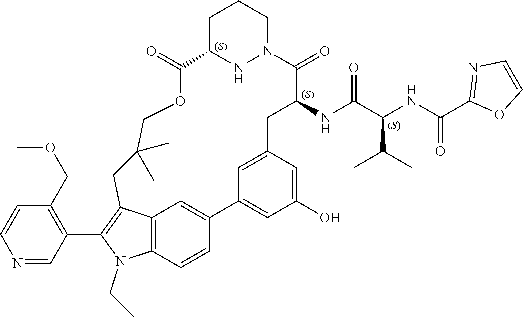 |
| AA57 | 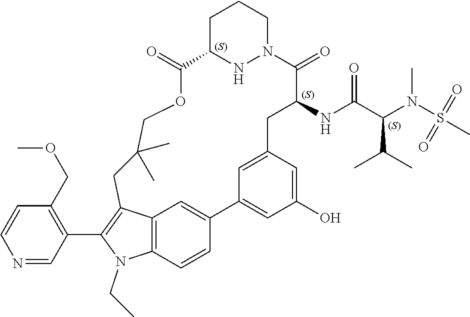 |
| AA58 | 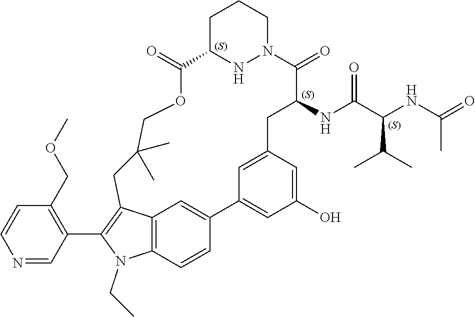 |
| AA59 | 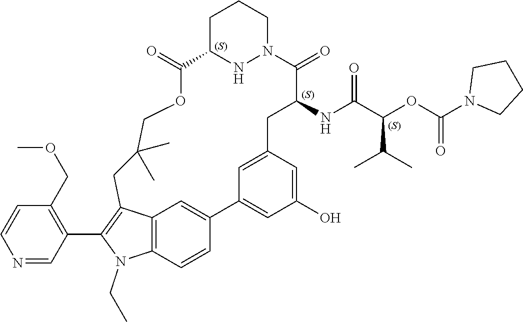 |
| AA60 | 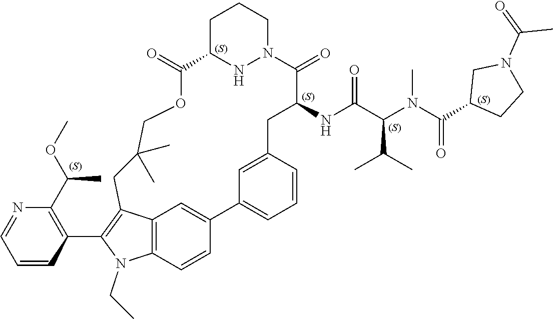 |
| AA61 | 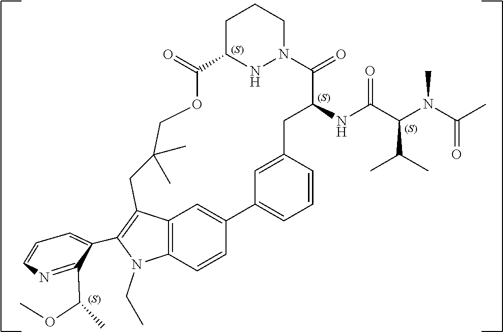 |
| AA62 | 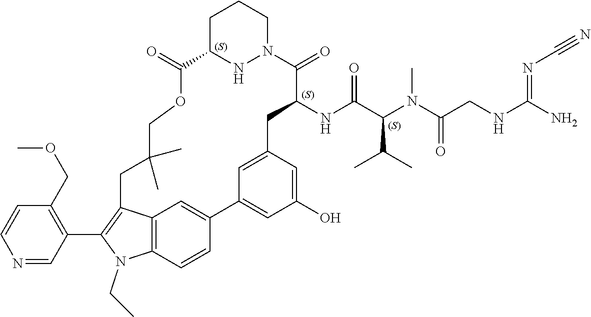 |
| AA63 | 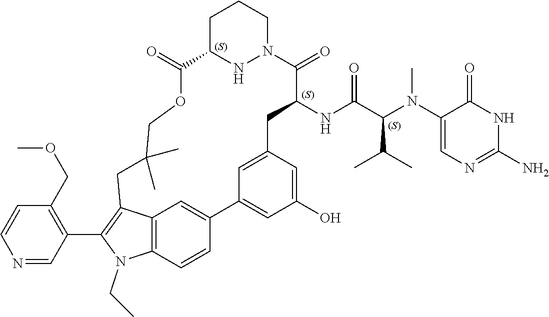 |
| AA64 | 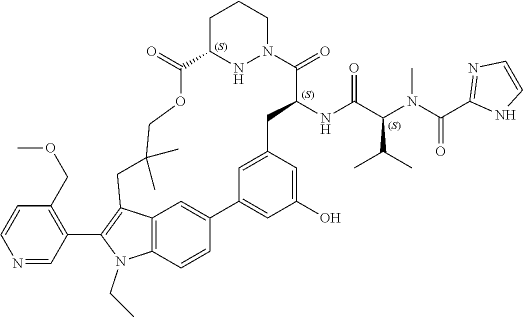 |
| AA65 | 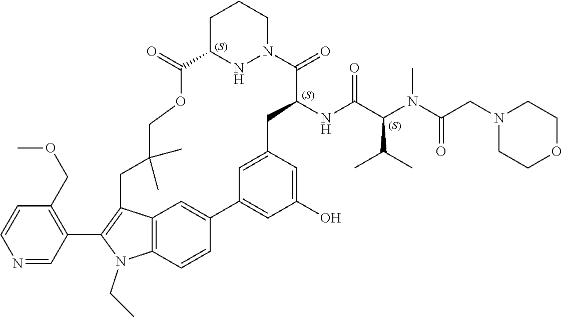 |
| AA66 | 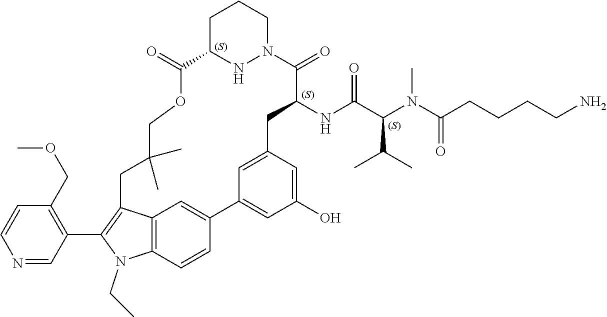 |
| AA67 | 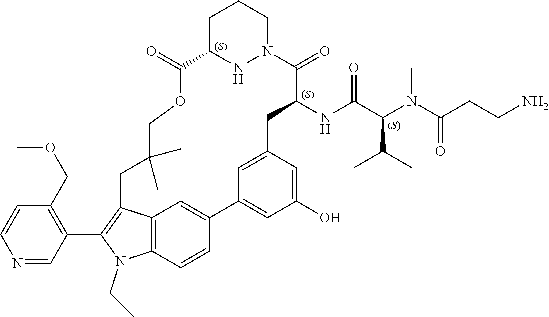 |
| AA68 | 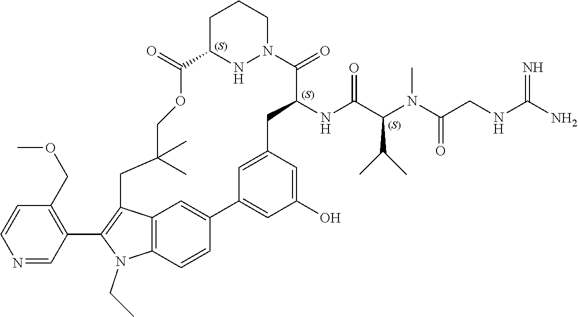 |
| AA69 | 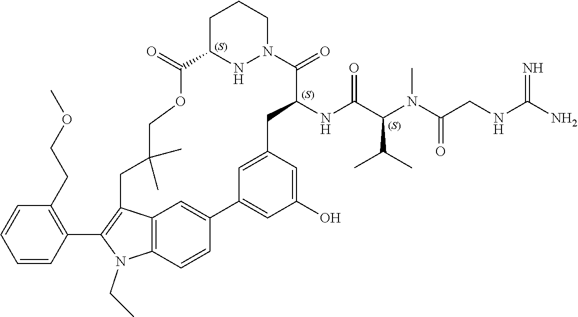 |
| AA70 | 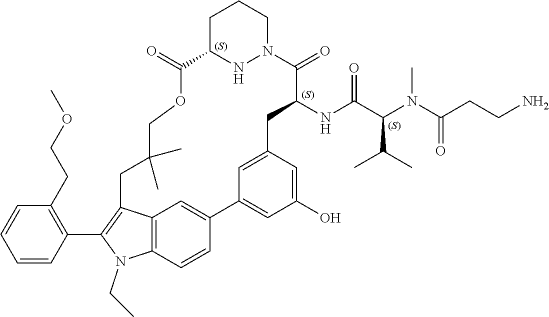 |
| AA71 | 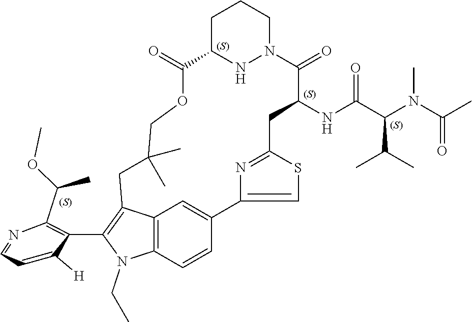 |
| AA72 | 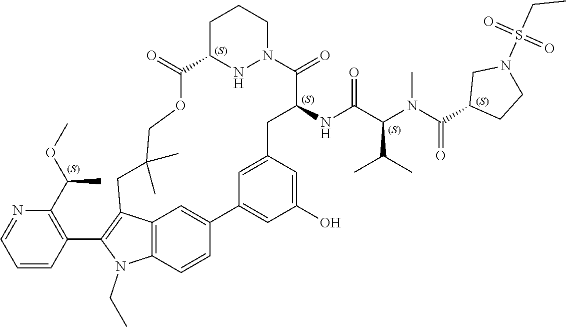 |
| AA73 | 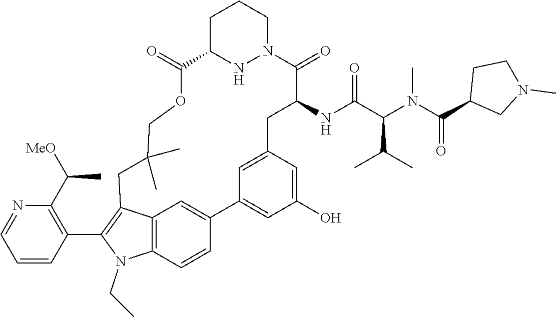 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA74 | 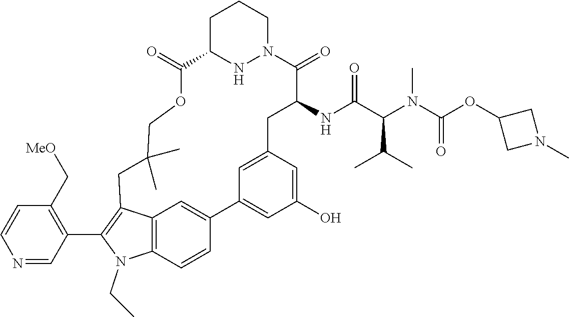 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| AA75 | 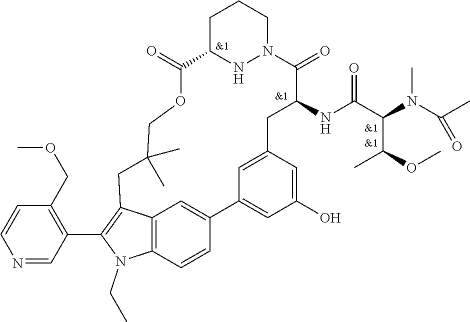 [SMILES: CCn1c(-c2cnccc2COC)c2c3cc(ccc31)-c1cc(O)cc(c1)C[C@H](NC(=O)[C@H]([C@H](C)OC)N(C)C(C)=O)C(=O)N1CCC[C@H](N1)C(=O)OCC(C)(C)C2] |
| AA76 | 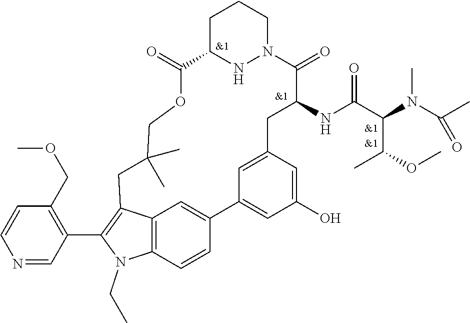 [SMILES: CCn1c(-c2cnccc2COC)c2c3cc(ccc31)-c1cc(O)cc(c1)C[C@H](NC(=O)[C@H]([C@@H](C)OC)N(C)C(C)=O)C(=O)N1CCC[C@H](N1)C(=O)OCC(C)(C)C2] |
| AA77 | 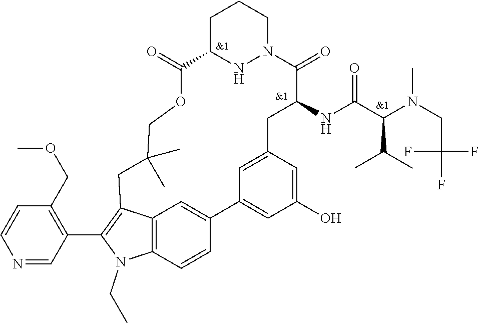 [SMILES: CCn1c(-c2cnccc2COC)c2c3cc(ccc31)-c1cc(O)cc(c1)C[C@H](NC(=O)[C@H](C(C)C)N(C)CC(F)(F)F)C(=O)N1CCC[C@H](N1)C(=O)OCC(C)(C)C2] |
| AA78 | 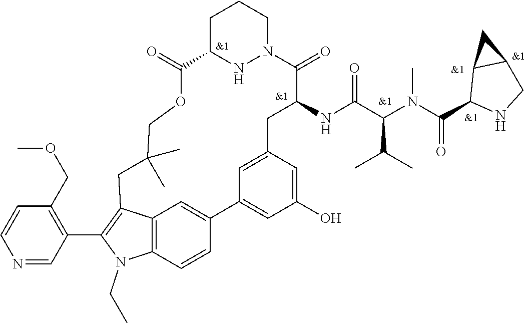 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NC[C@@H]3C[C@@H]31)C(=O)N1CCC[C@H](N1)C(=O)OCC(C)(C)C2] |
| AA79 | 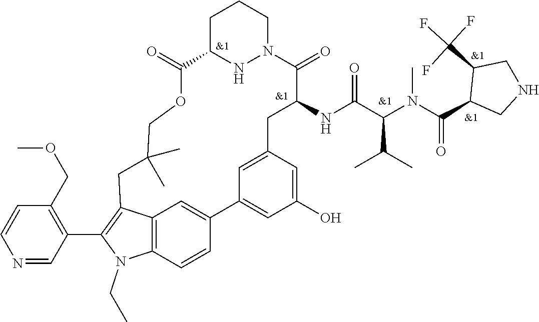 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1C(F)(F)F)C(=O)N1CCC[C@H](N1)C(=O)OCC(C)(C)C2] |
| AA80 | 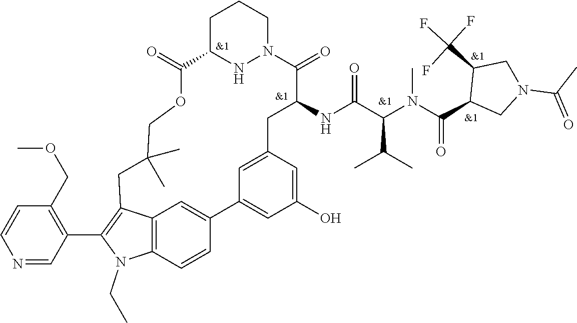 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CN(C(C)=O)C[C@@H]1C(F)(F)F)C(=O)N1CCC[C@H](N1)C(=O)OCC(C)(C)C2] |
| AA81 | 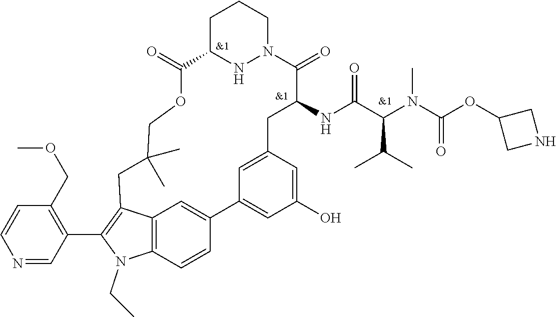 [SMILES: CCn1c(-c2cnccc2COC)c2c3cc(ccc31)-c1cc(O)cc(c1)C[C@H](NC(=O)[C@H](C(C)C)N(C)C(=O)OC1CNC1)C(=O)N1CCC[C@H](N1)C(=O)OCC(C)(C)C2] |
| AA82 | 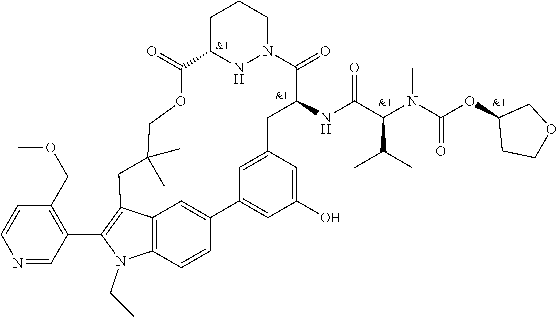 [SMILES: CCn1c(-c2cnccc2COC)c2c3cc(ccc31)-c1cc(O)cc(c1)C[C@H](NC(=O)[C@H](C(C)C)N(C)C(=O)O[C@@H]1CCOC1)C(=O)N1CCC[C@H](N1)C(=O)OCC(C)(C)C2] |
| AA83 | 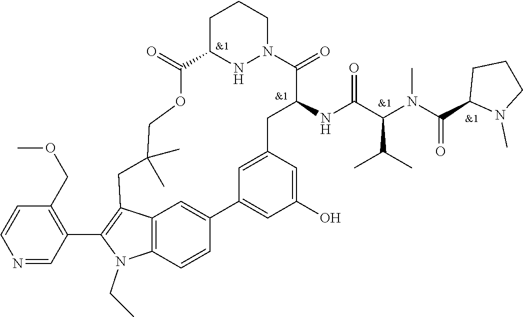 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCN1C)C(=O)N1CCC[C@H](N1)C(=O)OCC(C)(C)C2] |
| AA84 | 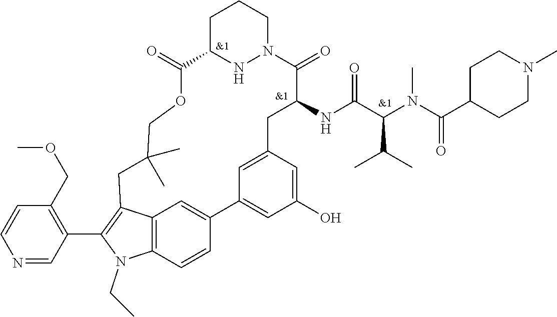 [SMILES: CCn1c(-c2cnccc2COC)c2c3cc(ccc31)-c1cc(O)cc(c1)C[C@H](NC(=O)[C@H](C(C)C)N(C)C(=O)C1CCN(C)CC1)C(=O)N1CCC[C@H](N1)C(=O)OCC(C)(C)C2] |
| AA85 | 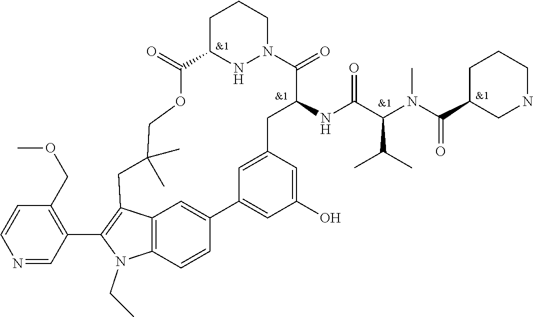 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCNC1)C(=O)N1CCC[C@H](N1)C(=O)OCC(C)(C)C2] |
| AA86 | 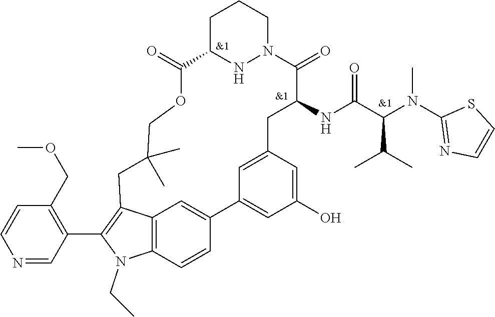 [SMILES: CCn1c(-c2cnccc2COC)c2c3cc(ccc31)-c1cc(O)cc(c1)C[C@H](NC(=O)[C@H](C(C)C)N(C)c1nccs1)C(=O)N1CCC[C@H](N1)C(=O)OCC(C)(C)C2] |
| AA87 | 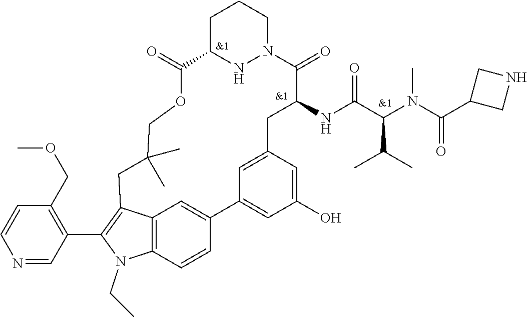 [SMILES: CCn1c(-c2cnccc2COC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| AA88 | 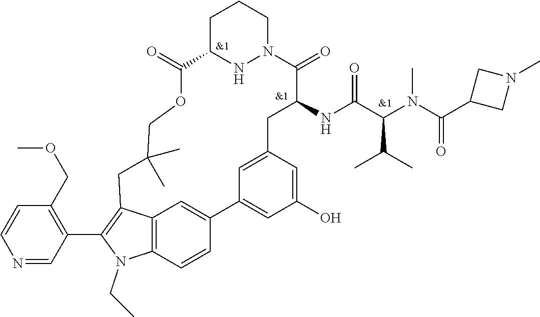 [SMILES: CCn1c(-c2cnccc2COC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| AA89 | 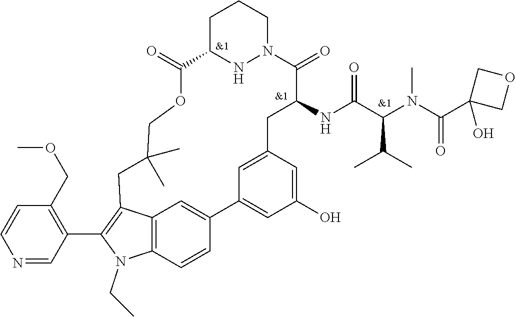 [SMILES: CCn1c(-c2cnccc2COC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| AA90 | 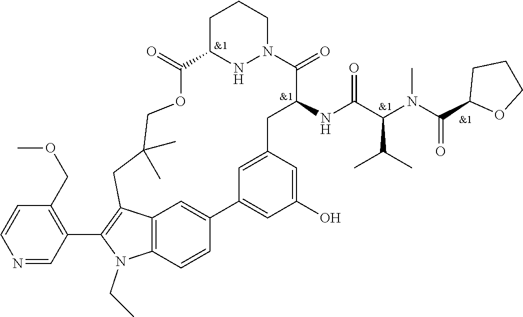 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCO1)C(=O)N1CCC[C@H](N1)C(=O)OCC(C)(C)C2] |
| AA91 | 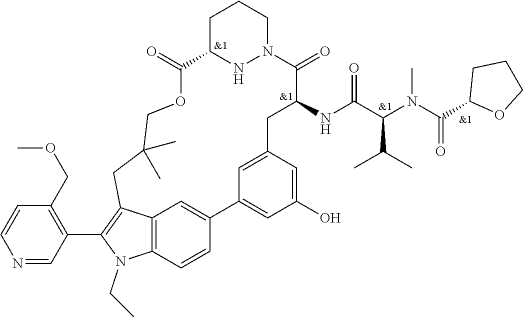 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCO1)C(=O)N1CCC[C@H](N1)C(=O)OCC(C)(C)C2] |
| AA92 | 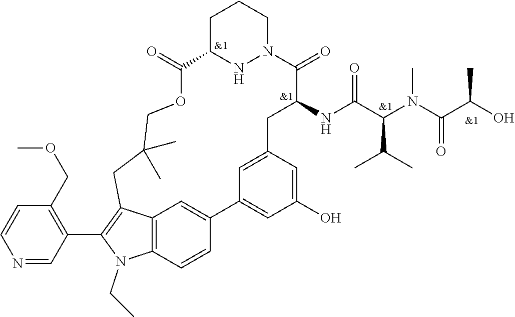 [SMILES: CCn1c(-c2cnccc2COC)c2c3cc(ccc31)-c1cc(O)cc(c1)C[C@H](NC(=O)[C@H](C(C)C)N(C)C(=O)[C@@H](C)O)C(=O)N1CCC[C@H](N1)C(=O)OCC(C)(C)C2] |
| AA93 | 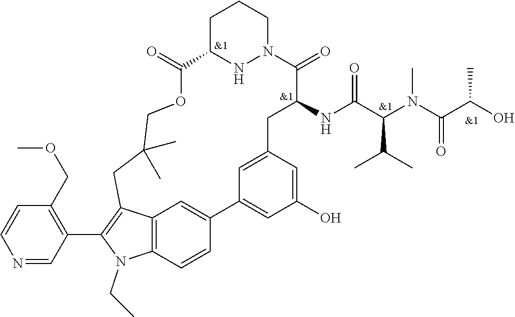 [SMILES: CCn1c(-c2cnccc2COC)c2c3cc(ccc31)-c1cc(O)cc(c1)C[C@H](NC(=O)[C@H](C(C)C)N(C)C(=O)[C@H](C)O)C(=O)N1CCC[C@H](N1)C(=O)OCC(C)(C)C2] |
| AA94 | 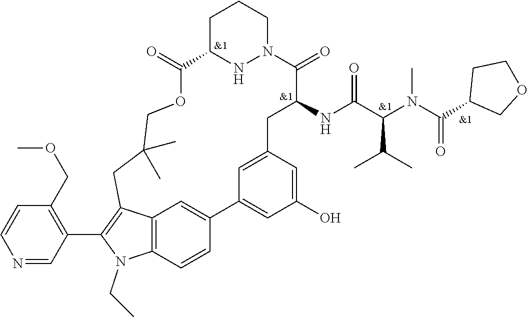 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCOC1)C(=O)N1CCC[C@H](N1)C(=O)OCC(C)(C)C2] |
| AA95 | 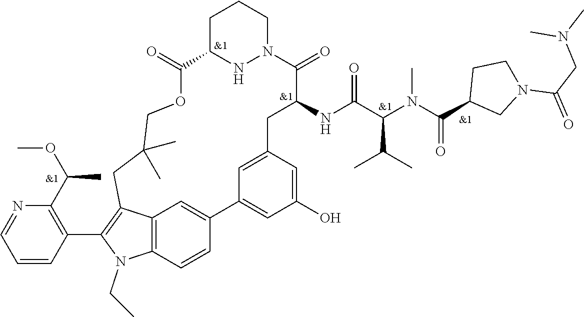 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA96 | 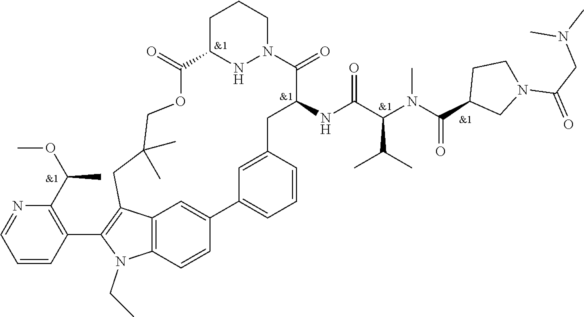 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA97 | 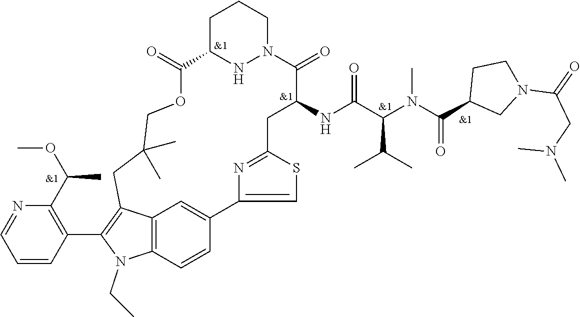 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA98 | 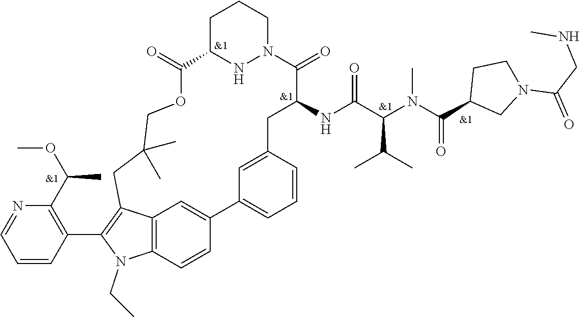 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA99 | 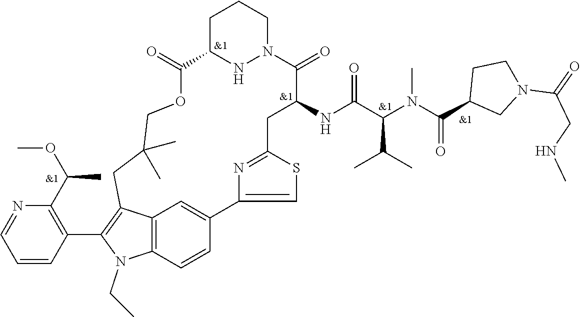 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA100 | 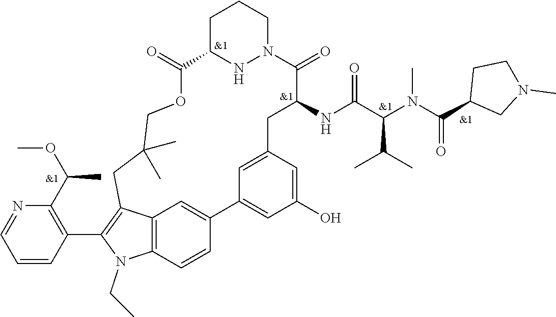 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA101 | 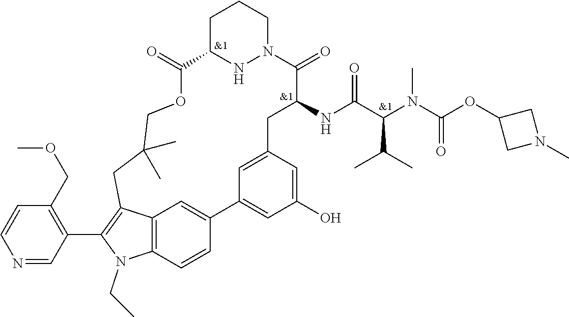 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| AA102 | 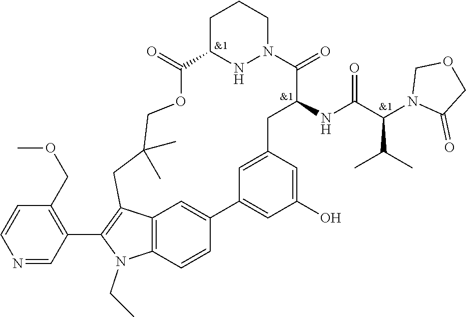 [SMILES: CCn1c(-c2cnccc2COC)c2c3cc(ccc31)-c1cc(O)cc(c1)C[C@H](NC(=O)[C@H](C(C)C)N1COCC1=O)C(=O)N1CCC[C@H](N1)C(=O)OCC(C)(C)C2] |
| AA103 | 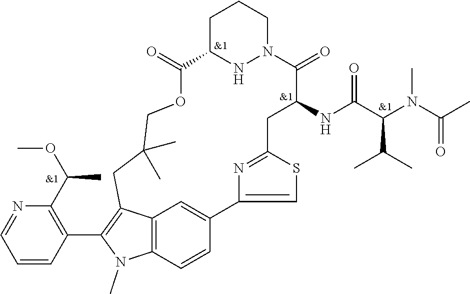 [SMILES: CO[C@@H](C)c1ncccc1-c1c2c3cc(ccc3n1C)-c1csc(n1)C[C@H](NC(=O)[C@H](C(C)C)N(C)C(C)=O)C(=O)N1CCC[C@H](N1)C(=O)OCC(C)(C)C2] |
| AA104 | 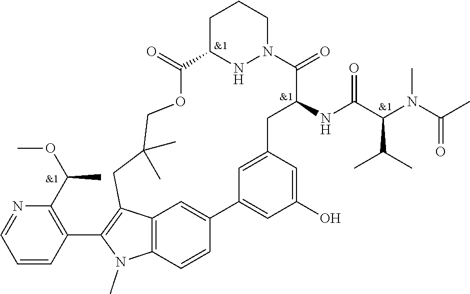 [SMILES: CO[C@@H](C)c1ncccc1-c1c2c3cc(ccc3n1C)-c1cc(O)cc(c1)C[C@H](NC(=O)[C@H](C(C)C)N(C)C(C)=O)C(=O)N1CCC[C@H](N1)C(=O)OCC(C)(C)C2] |
| AA105 | 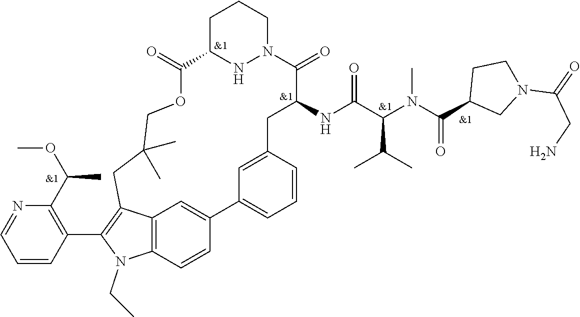 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| AA106 | 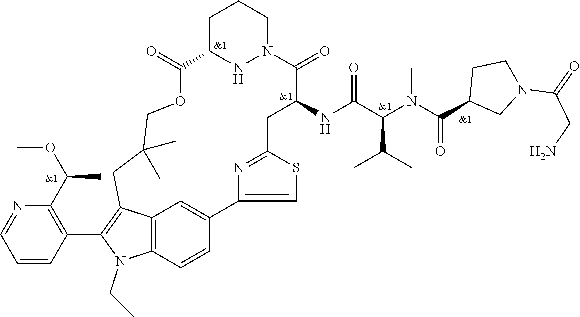 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| AA107 | 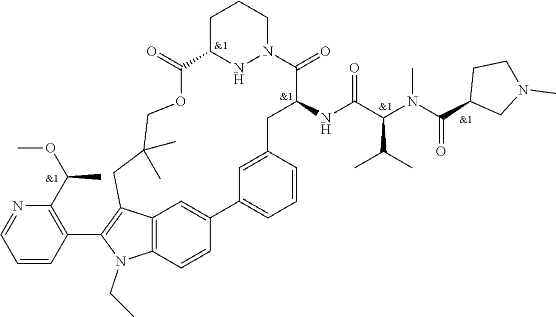 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA108 | 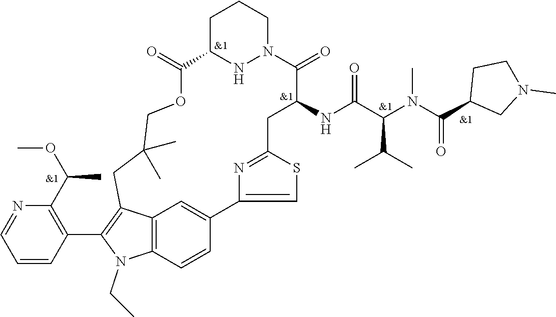 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C)C1)C(=O)N1CCC[C@H](N1)C(=O)OCC(C)(C)C2] |
| AA109 | 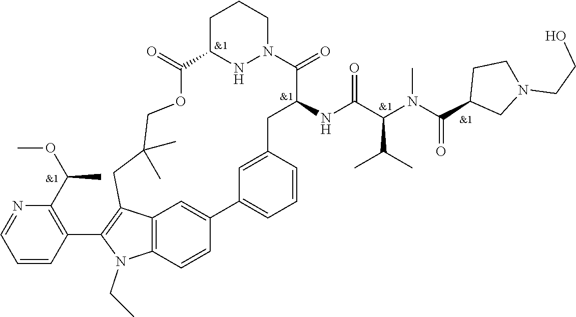 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA110 | 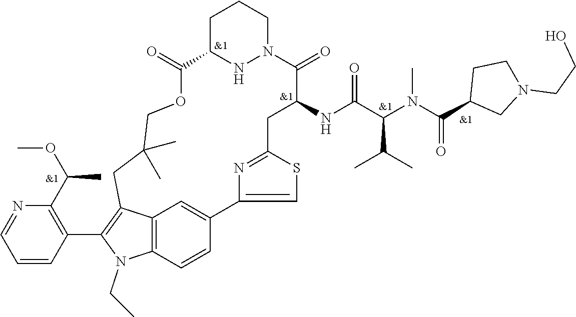 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA111 | 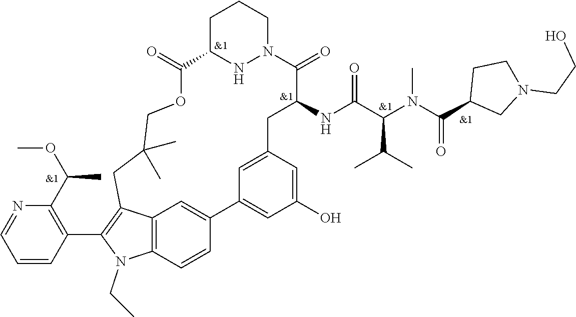 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA112 | 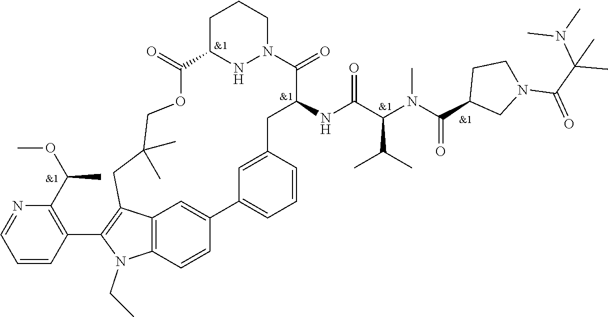 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA113 | 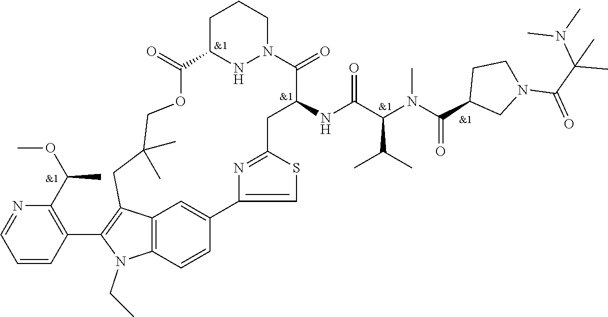 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA114 | 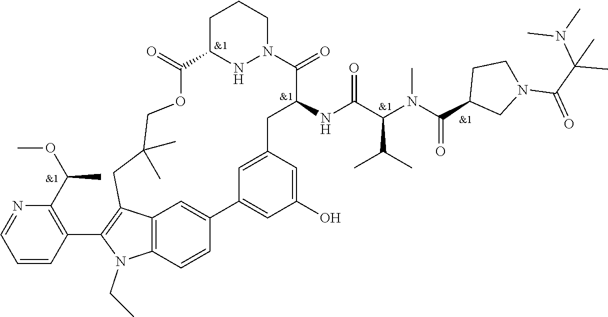 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA115 | 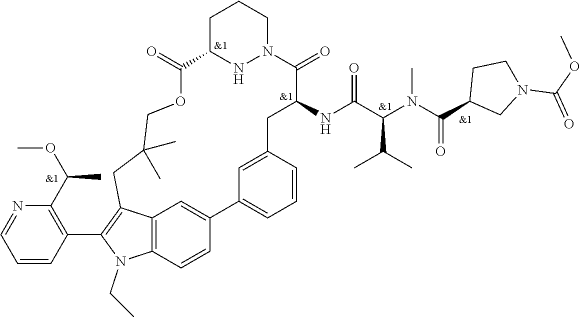 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| AA116 | 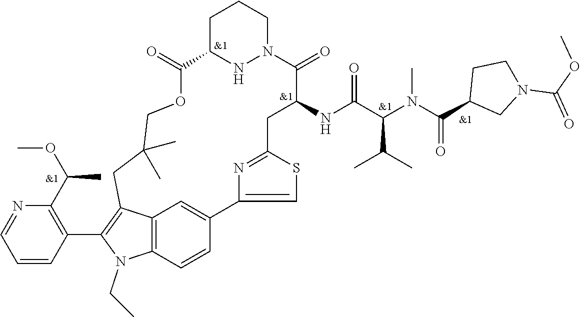 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| AA117 | 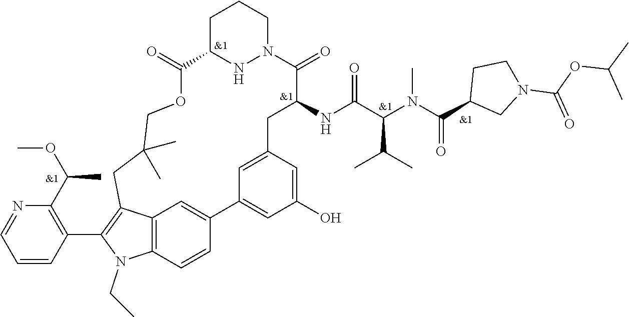 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)OC(C)C)C1)C(=O)N1CCC[C@H](N1)C(=O)OCC(C)(C)C2] |
| AA118 | 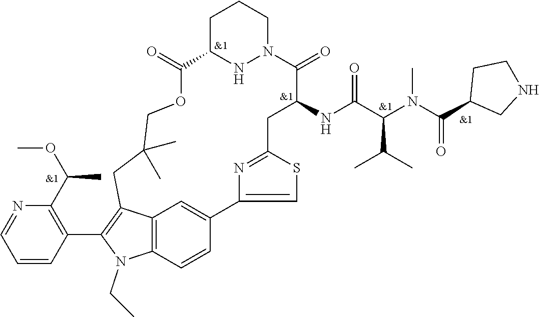 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCNC1)C(=O)N1CCC[C@H](N1)C(=O)OCC(C)(C)C2] |
| AA119 | 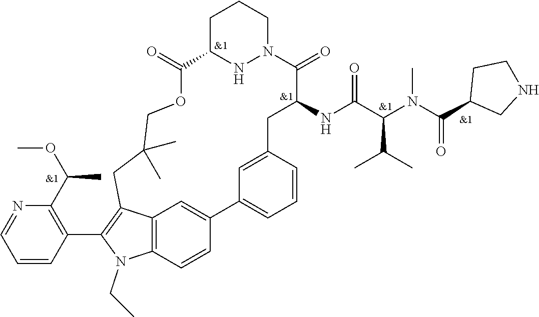 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCNC1)C(=O)N1CCC[C@H](N1)C(=O)OCC(C)(C)C2] |
| AA120 | 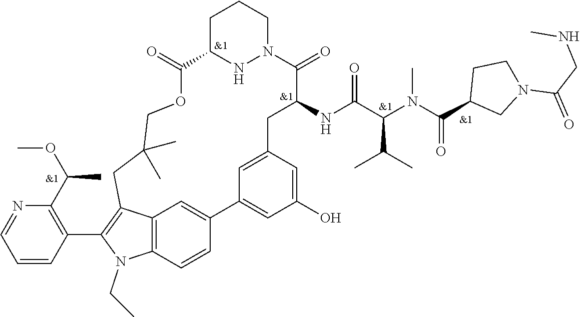 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA121 | 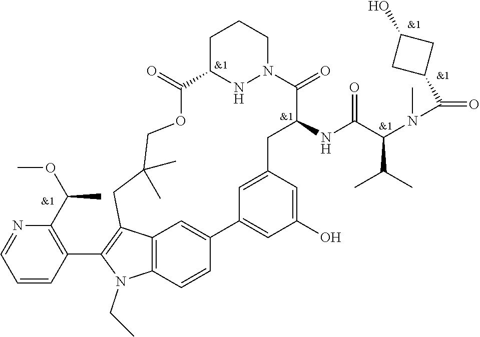 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H](O)C1)C(=O)N1CCC[C@H](N1)C(=O)OCC(C)(C)C2] |
| AA122 | 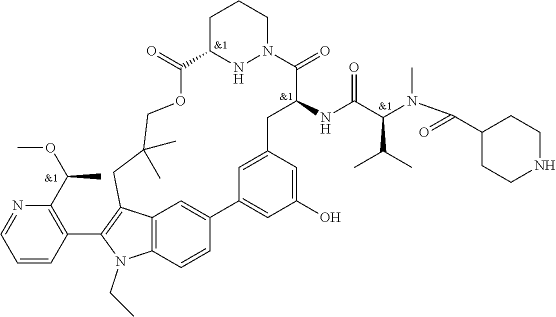 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNCC1)C(=O)N1CCC[C@H](N1)C(=O)OCC(C)(C)C2] |
| AA123 | 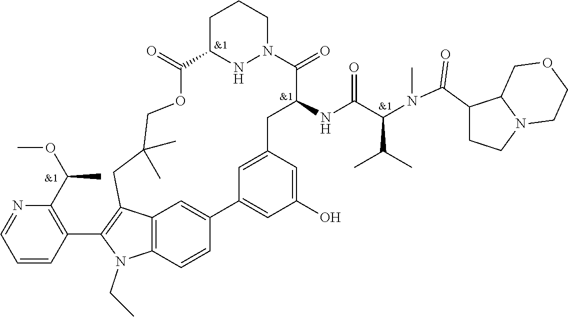 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCN3CCOCC13)C(=O)N1CCC[C@H](N1)C(=O)OCC(C)(C)C2] |
| AA124 | 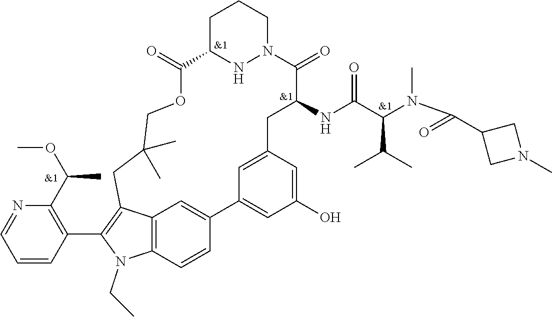 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| AA125 | 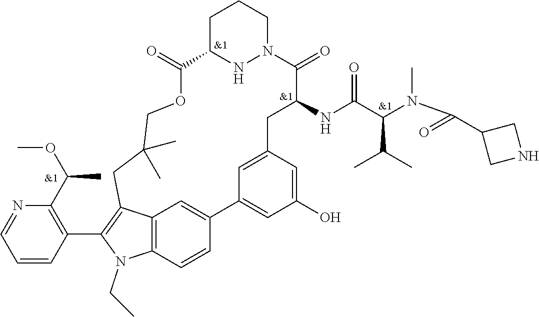 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| AA126 | 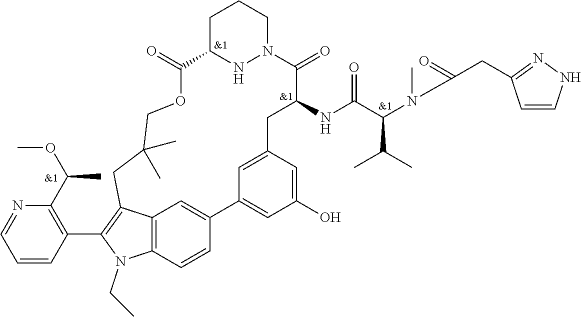 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1cc[nH]n1)C(=O)N1CCC[C@H](N1)C(=O)OCC(C)(C)C2] |
| AA127 | 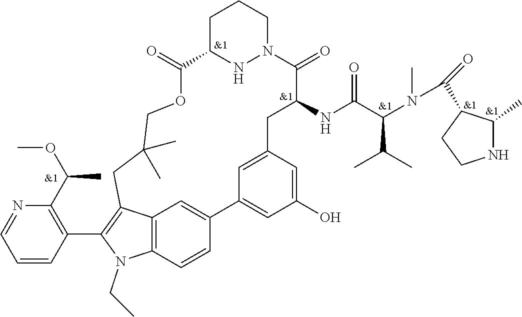 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN[C@H]1C)C(=O)N1CCC[C@H](N1)C(=O)OCC(C)(C)C2] |
| AA128 | 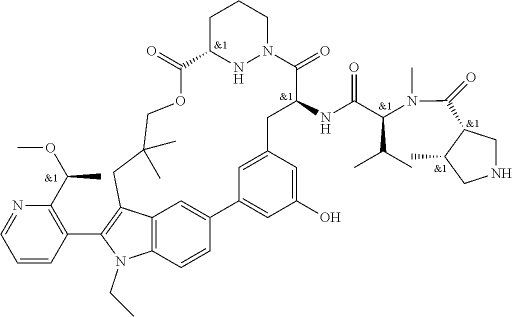 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1C)C(=O)N1CCC[C@H](N1)C(=O)OCC(C)(C)C2] |
| AA129 | 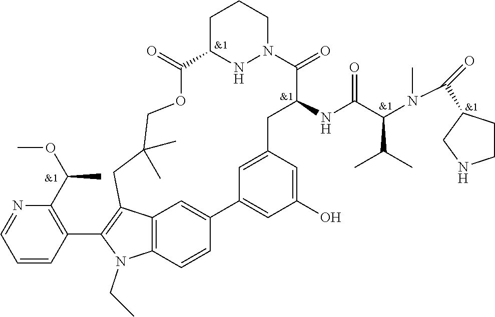 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CCNC1)C(=O)N1CCC[C@H](N1)C(=O)OCC(C)(C)C2] |
| AA130 | 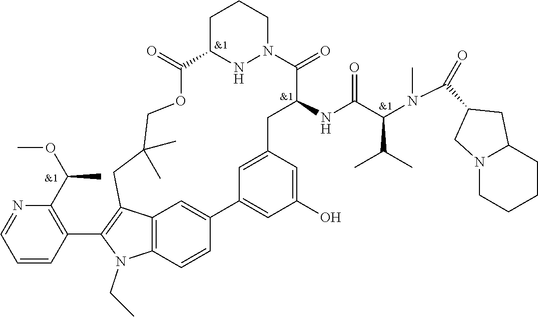 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CC3CCCCN3C1)C(=O)N1CCC[C@H](N1)C(=O)OCC(C)(C)C2] |
| AA131 | 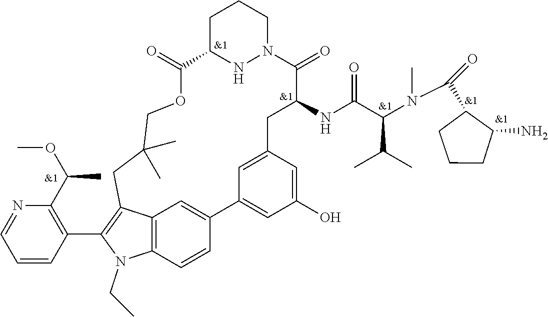 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCC[C@H]1N)C(=O)N1CCC[C@H](N1)C(=O)OCC(C)(C)C2] |
| AA132 | 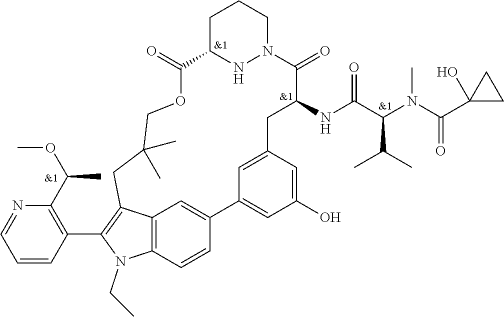 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)CC1)C(=O)N1CCC[C@H](N1)C(=O)OCC(C)(C)C2] |
| AA133 | 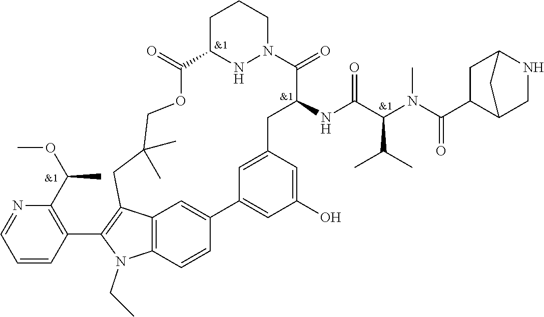 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CC1CN3)C(=O)N1CCC[C@H](N1)C(=O)OCC(C)(C)C2] |
| AA134 | 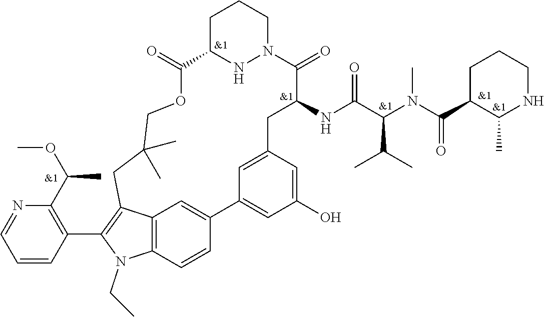 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCCN[C@@H]1C)C(=O)N1CCC[C@H](N1)C(=O)OCC(C)(C)C2] |
| AA135 | 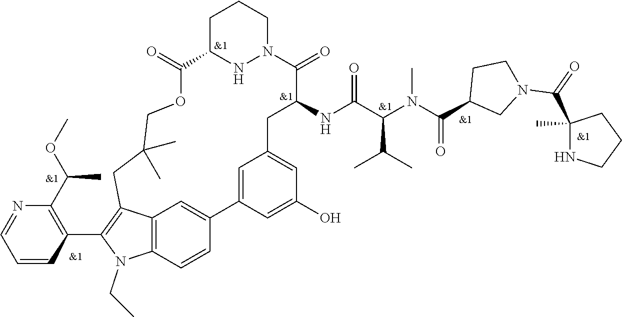 |
| AA136 | 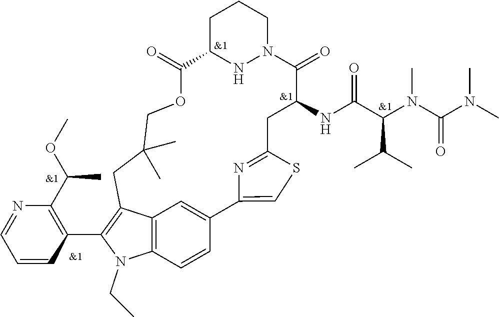 |
| AA137 | 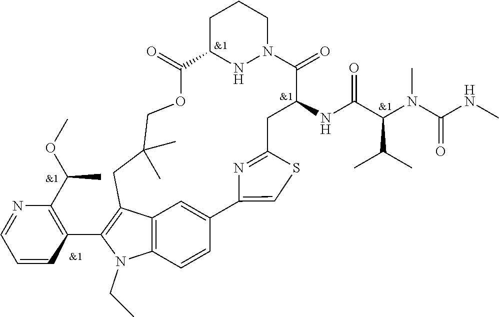 |
| AA138 | 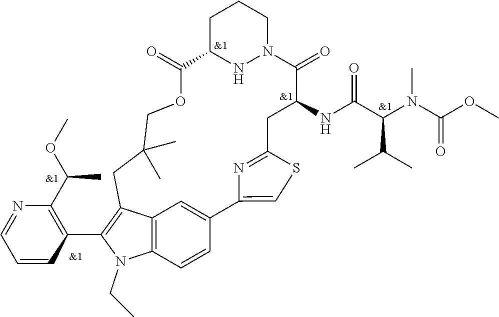 |
| AA139 | 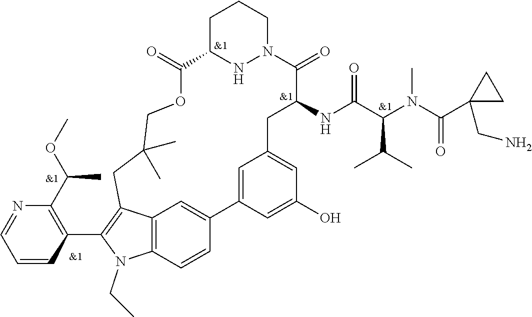 |
| AA140 | 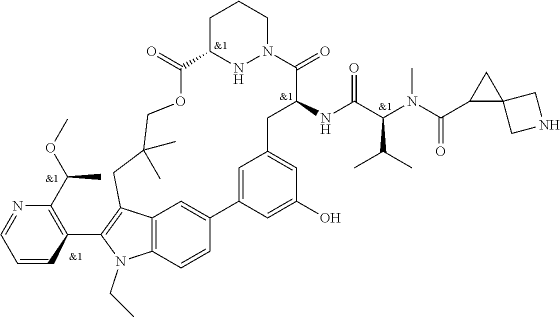 |
| AA141 | 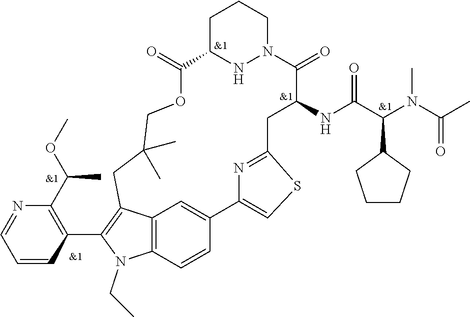 |
| AA142 | 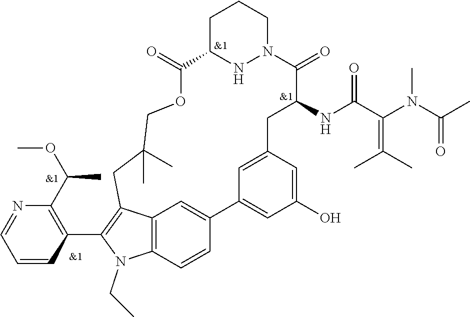 |
| AA143 | 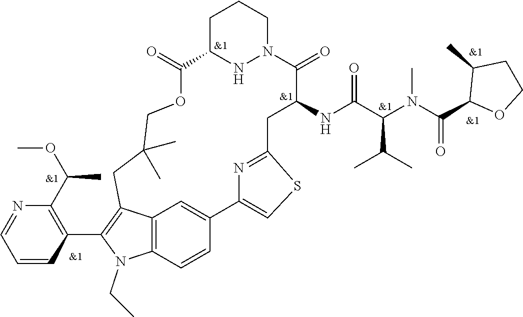 |
| AA144 | 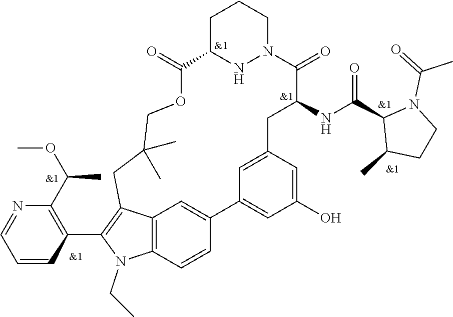 |
| AA145 | 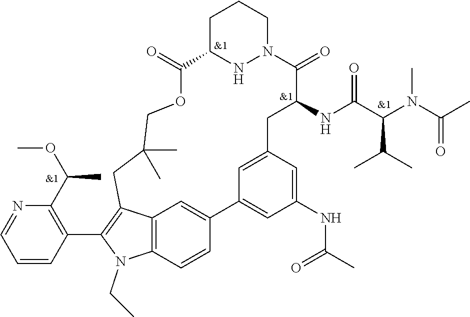 [SMILES: CCn1c(-c2cccnc2[C@H](C)OC)c2c3cc(ccc31)-c1cc(cc(NC(C)=O)c1)C[C@H](NC(=O)[C@H](C(C)C)N(C)C(C)=O)C(=O)N1CCC[C@H](N1)C(=O)OCC(C)(C)C2] |
| AA146 | 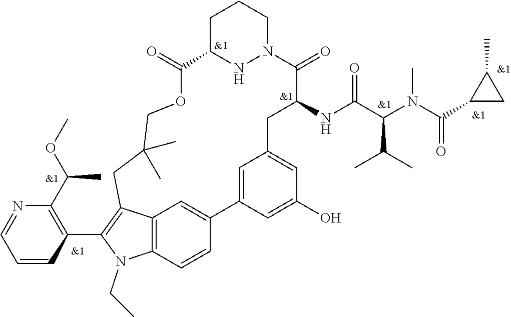 |
| AA147 | 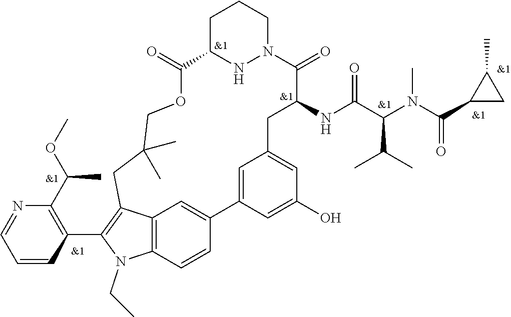 |
| AA148 | 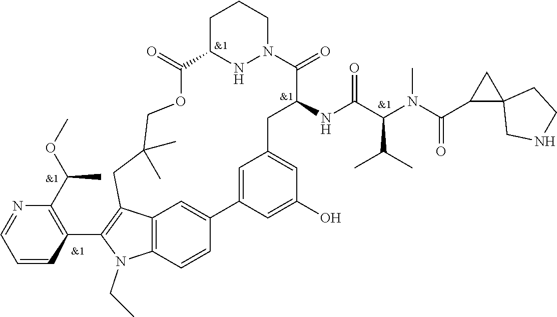 |
| AA149 | 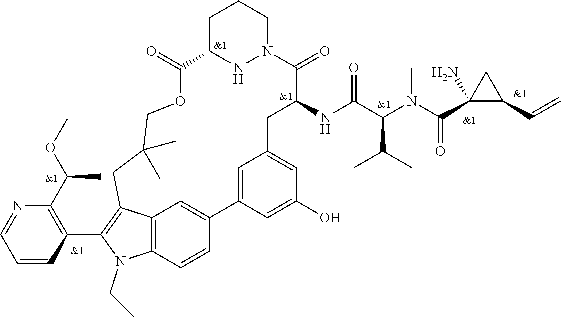 |
| AA150 | 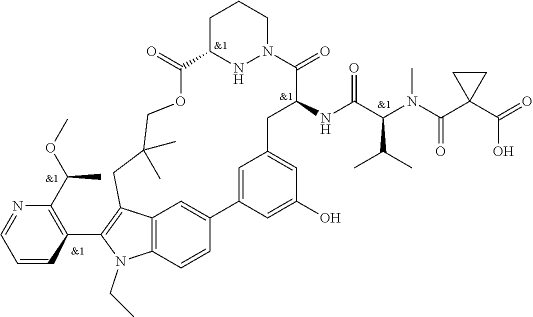 |
| AA151 | 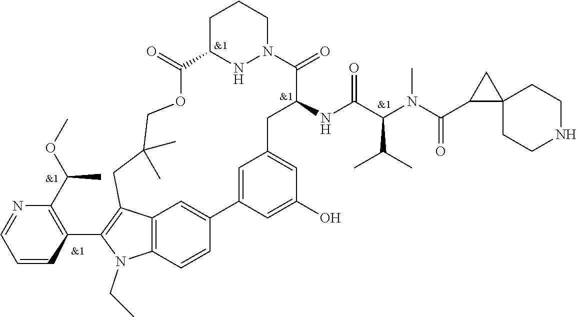 |
| AA152 | 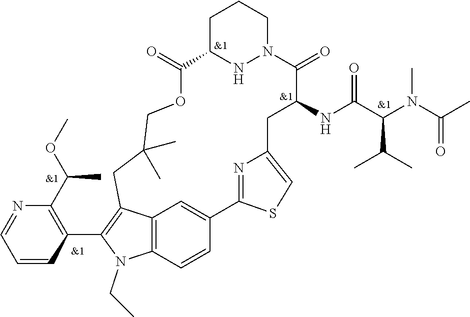 |
| AA153 | 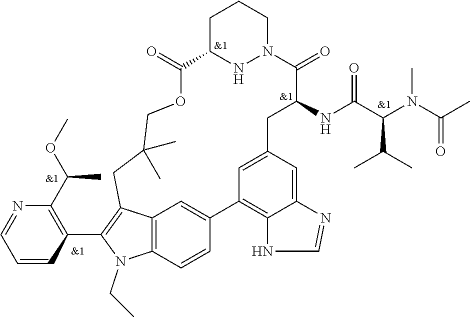 |
| AA154 | 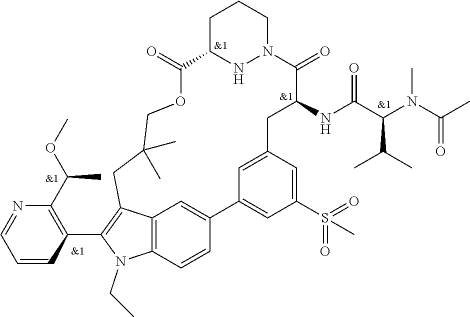 |
| AA155 | 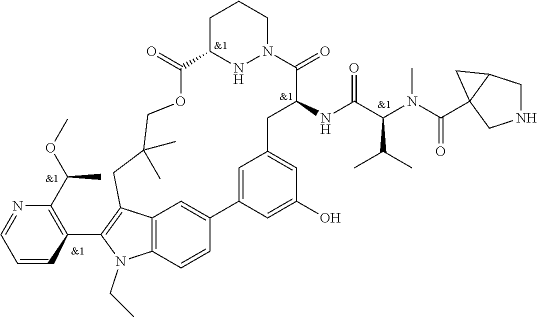 |
| AA156 | 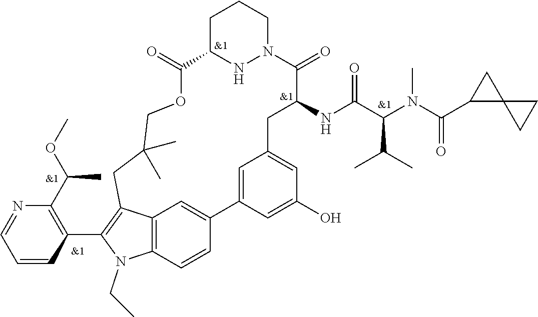 |
| AA157 | 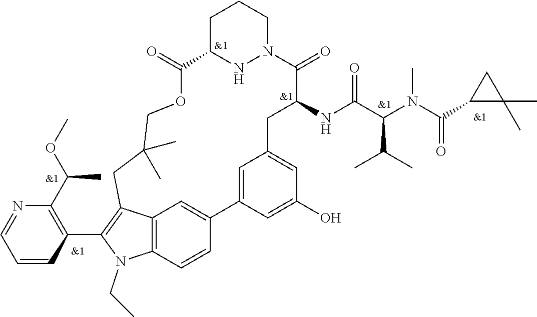 |
| AA158 | 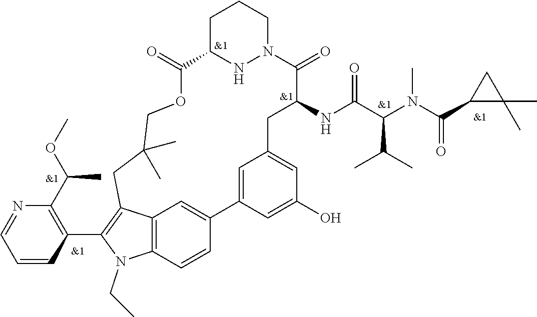 |
| AA159 | 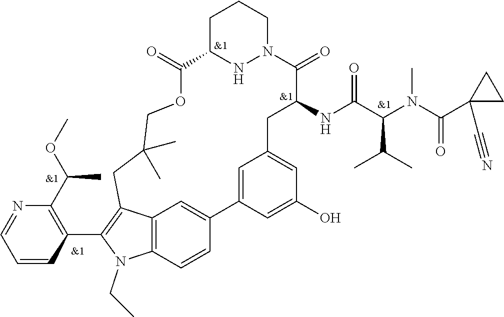 |
| AA160 | 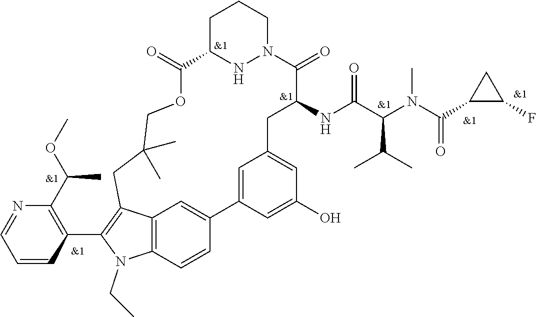 |
| AA161 | 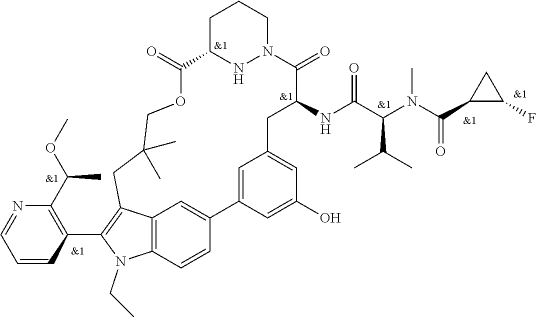 |
| AA162 | 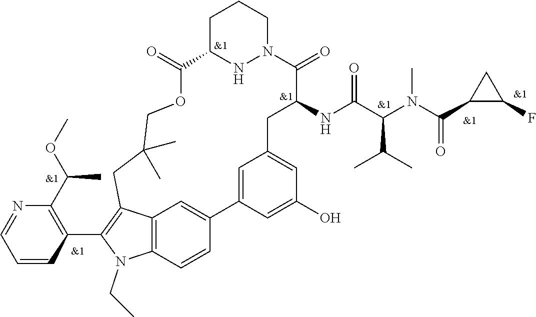 |
| AA163 | 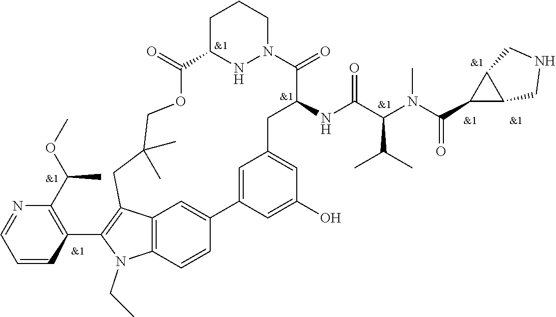 |
| AA164 | 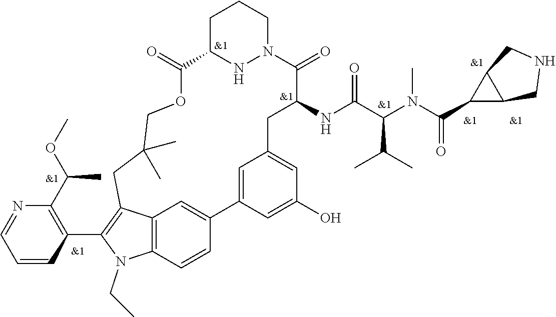 |
| AA165 | 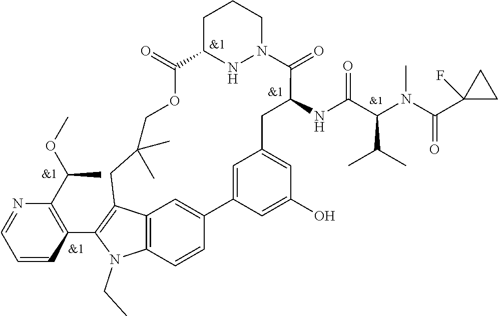 |
| AA166 | 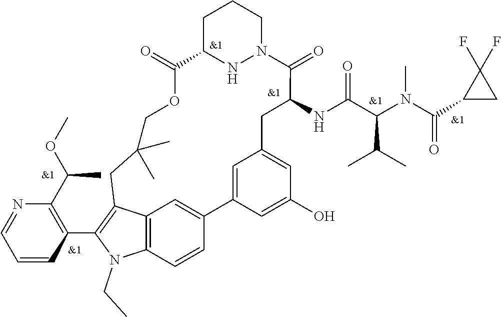 |
| AA167 | 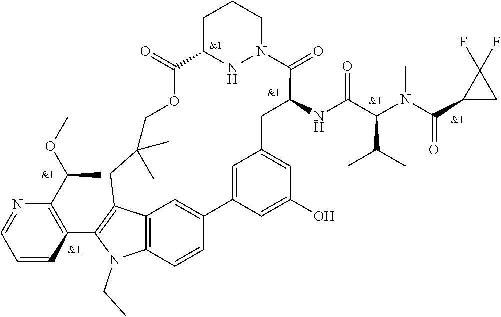 |
| AA168 | 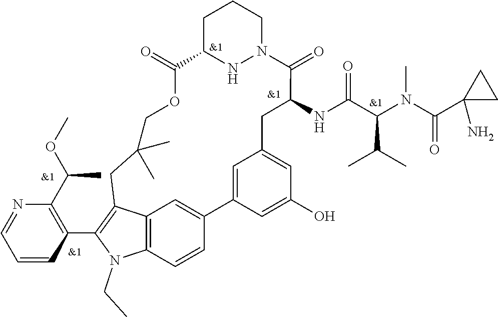 |
| AA169 | 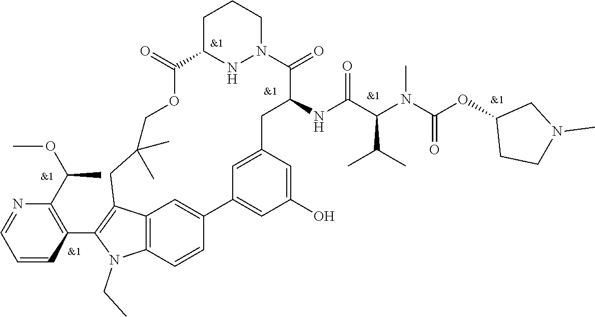 |
| AA170 | 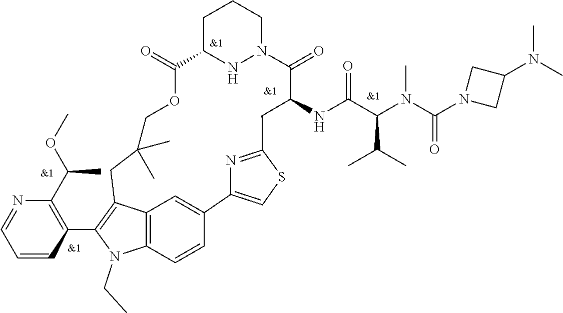 |
| AA171 | 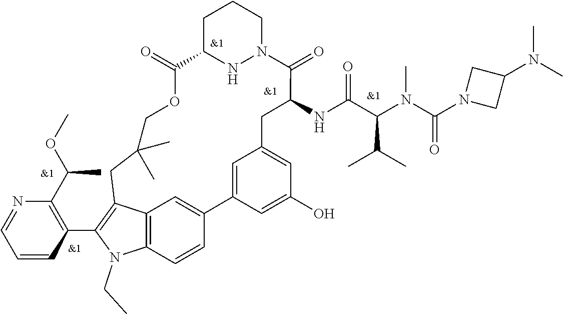 |
| AA172 | 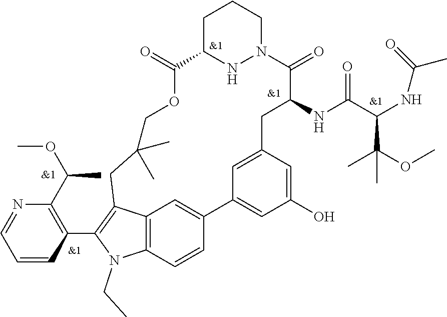 |
| AA173 | 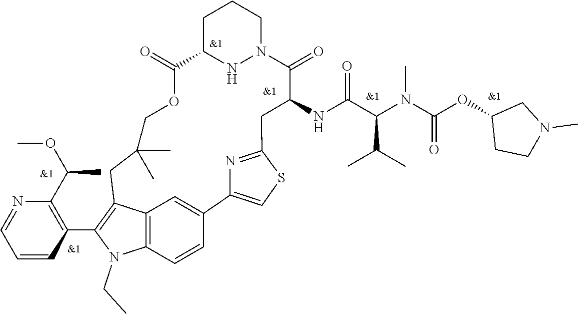 |
| AA174 | 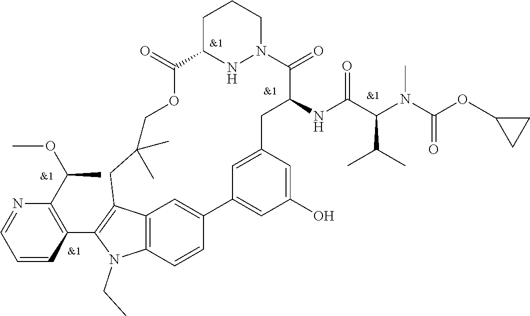 |
| AA175 | 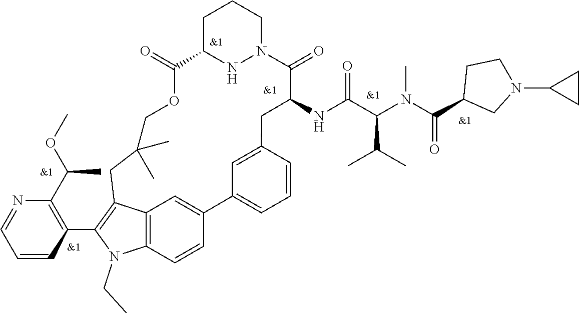 |
| AA176 | 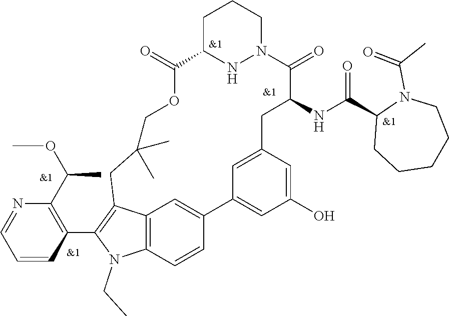 |
| AA177 | 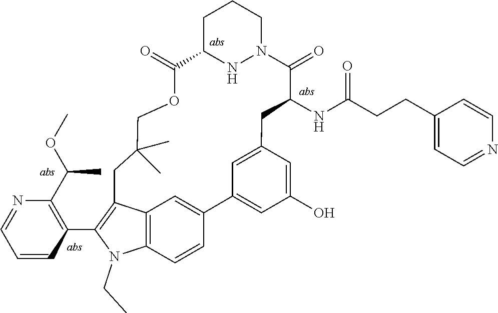 |
| AA178 | 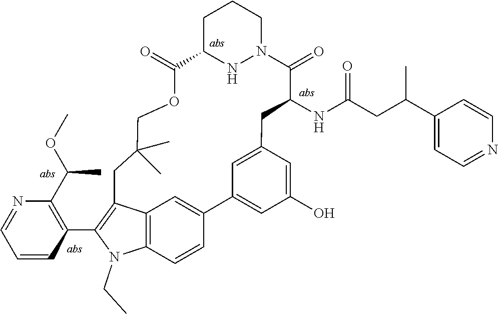 |
| AA179 | 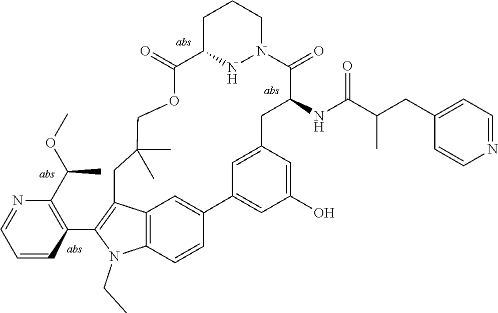 |
| AA180 | 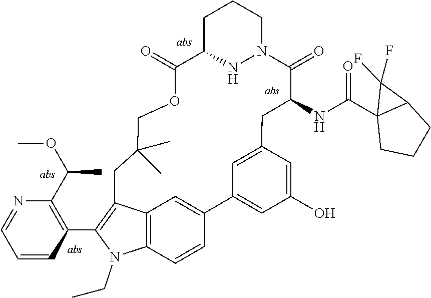 |
| AA181 | 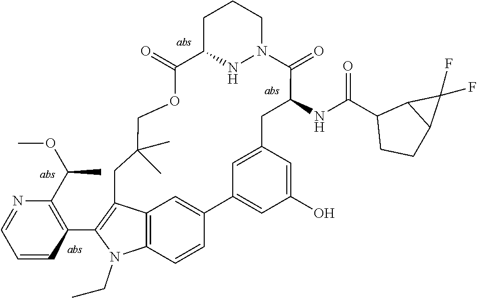 |
| AA182 | 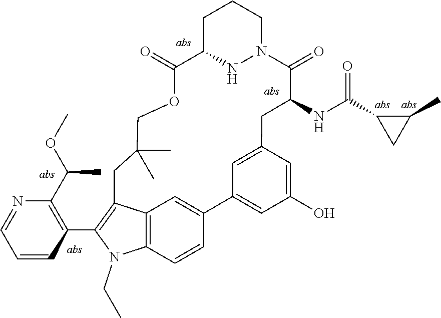 |
| AA183 | 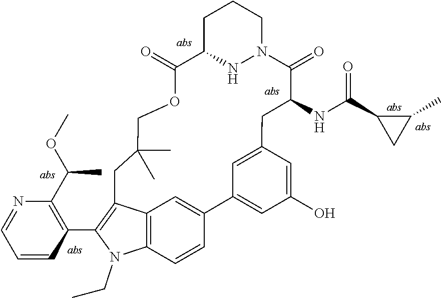 |
| AA184 | 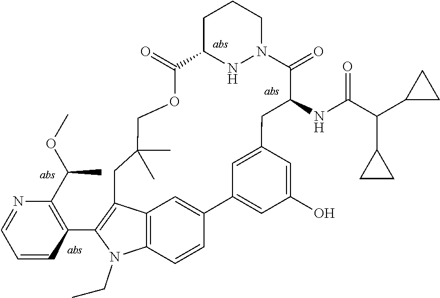 |
| AA185 | 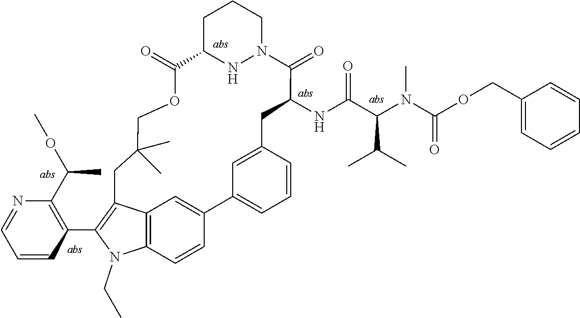 |
| AA186 | 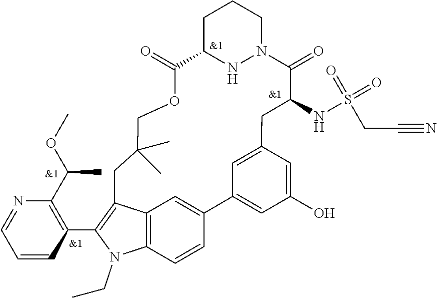 |
| AA187 | 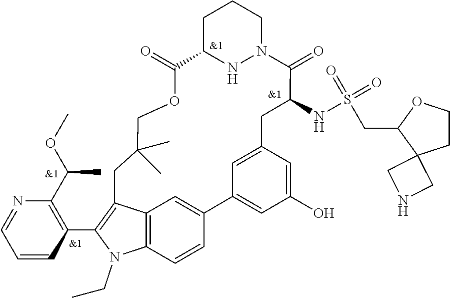 |
| AA188 | 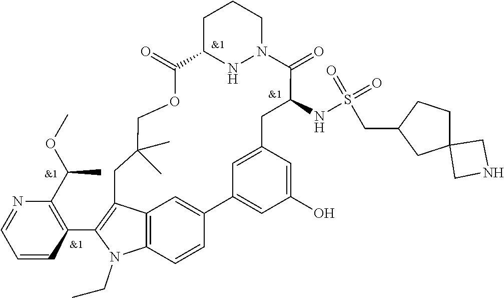 |
| AA189 | 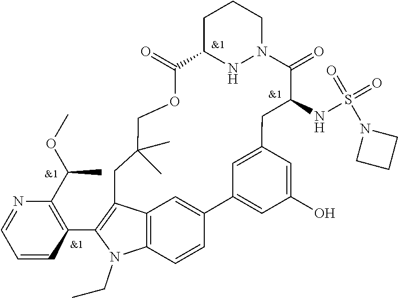 |
| AA190 | 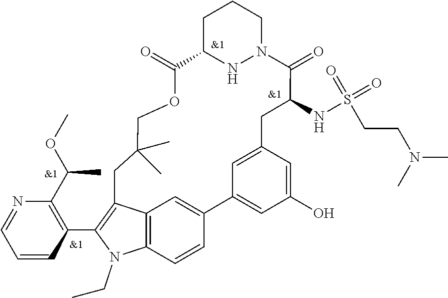 |
| AA191 | 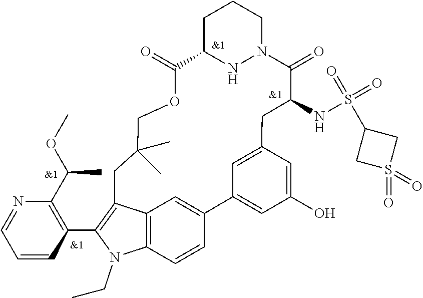 |
| AA192 | 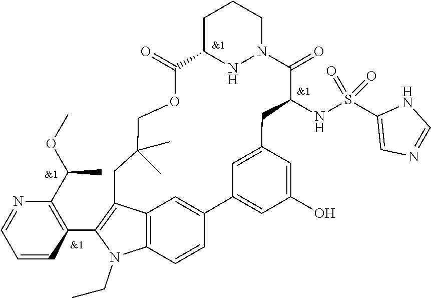 |
| AA193 | 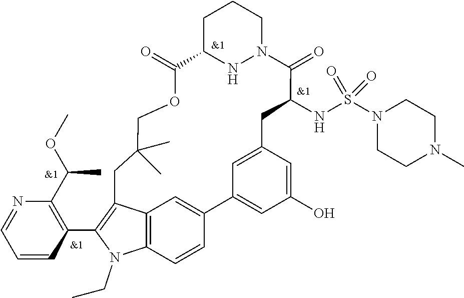 |
| AA194 | 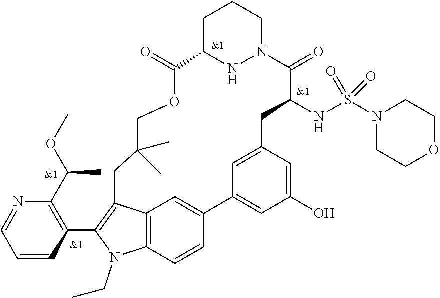 |
| AA195 | 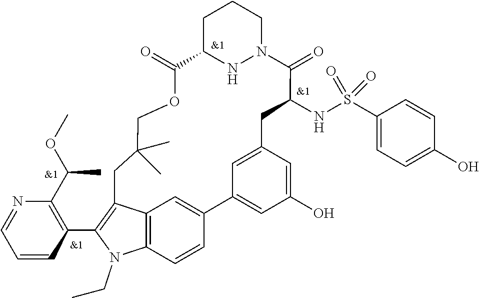 |
| AA196 | 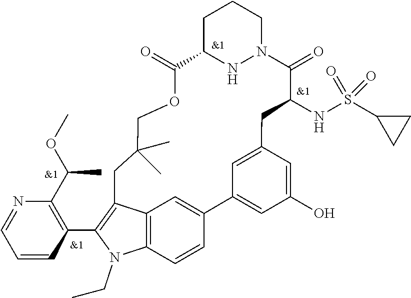 |
| AA197 | 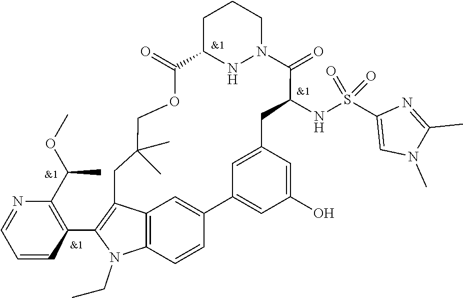 |
| AA198 | 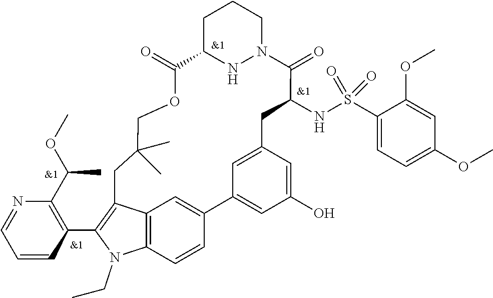 |
| AA199 | 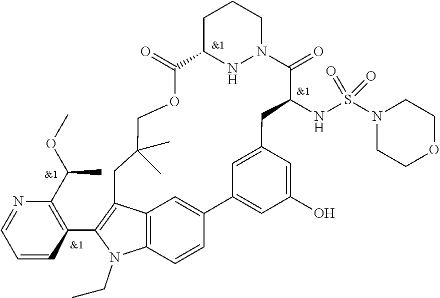 |
| AA200 | 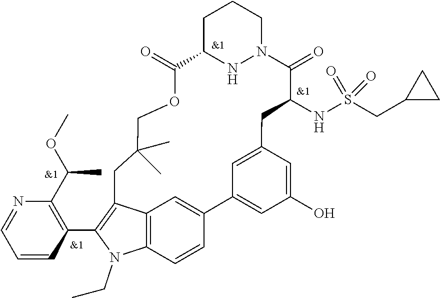 |
| AA201 | 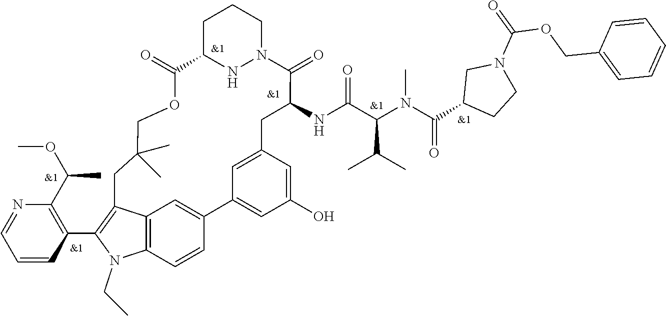 |
| AA202 | 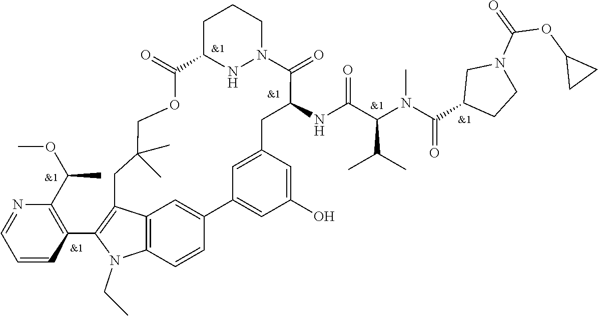 |
| AA203 | 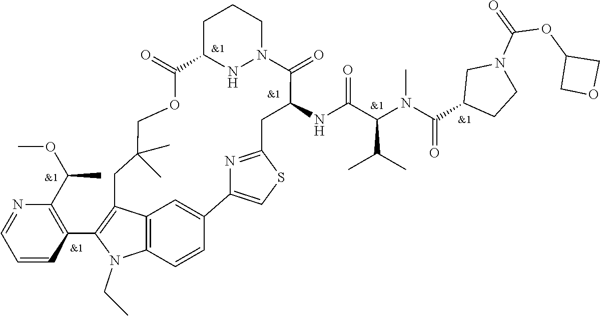 |
| AA204 | 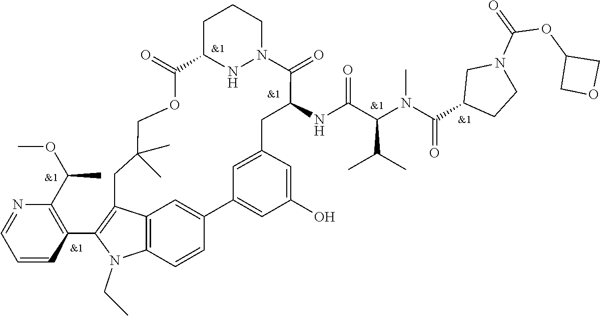 |
| AA205 | 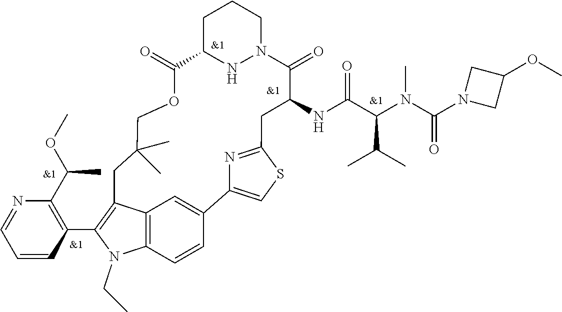 |
| AA206 | 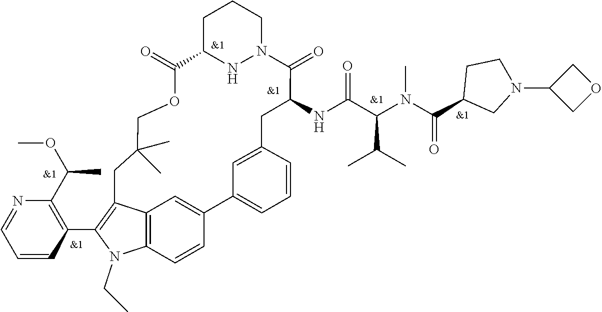 |
| AA207 | 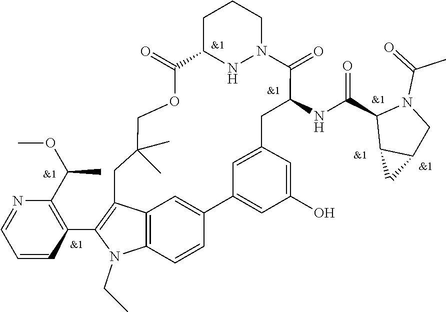 |
| AA208 | 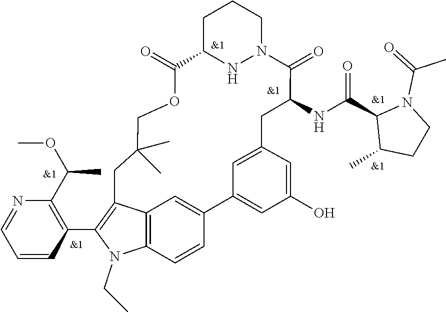 |
| AA209 | 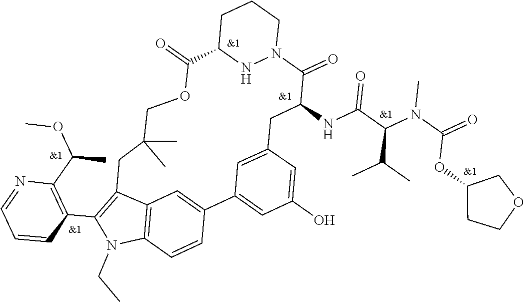 |
| AA210 | 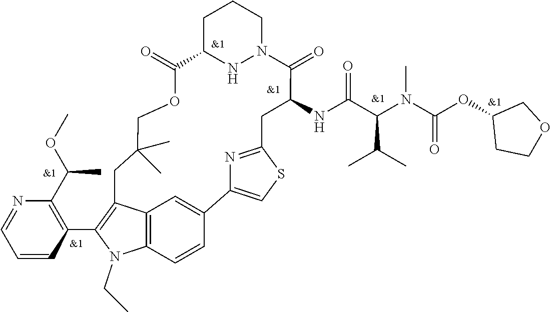 |
| AA211 | 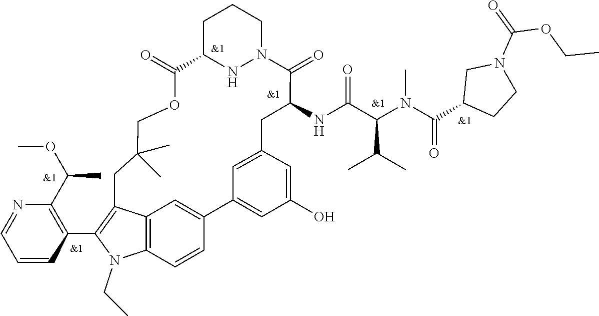 |
| AA212 | 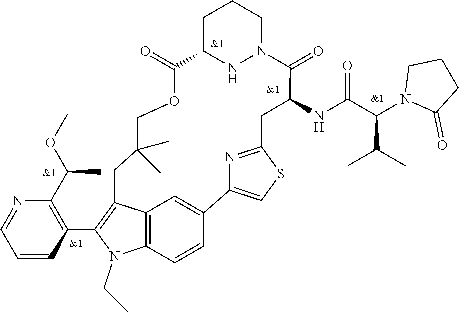 |
| AA213 | 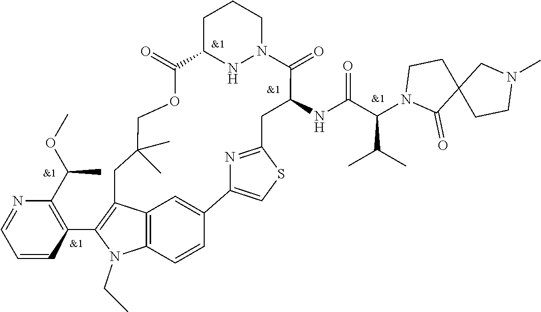 |
| AA214 | 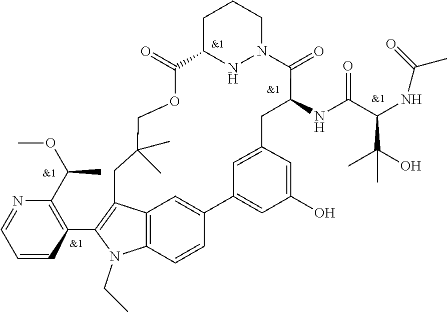 |
| AA215 | 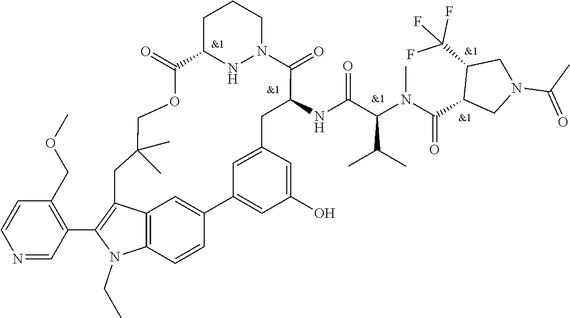 [SMILES: CCn1c(-c2cnccc2COC)c2c3cc(ccc31)-c1cc(O)cc(c1)C[C@H](NC(=O)[C@H](C(C)C)N(C)C(=O)[C@H]1CN(C(C)=O)C[C@H]1C(F)(F)F)C(=O)N1CCC[C@H](N1)C(=O)OCC(C)(C)C2] |
| AA216 | 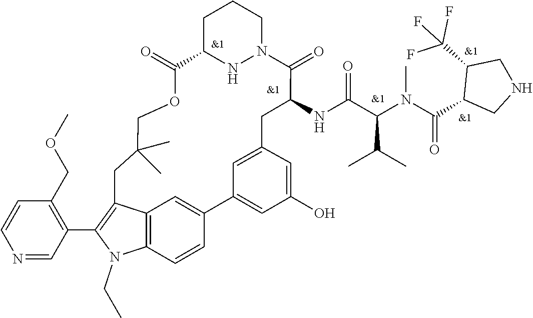 [SMILES: CCn1c(-c2cnccc2COC)c2c3cc(ccc31)-c1cc(O)cc(c1)C[C@H](NC(=O)[C@H](C(C)C)N(C)C(=O)[C@H]1CNC[C@H]1C(F)(F)F)C(=O)N1CCC[C@H](N1)C(=O)OCC(C)(C)C2] |
| AA217 | 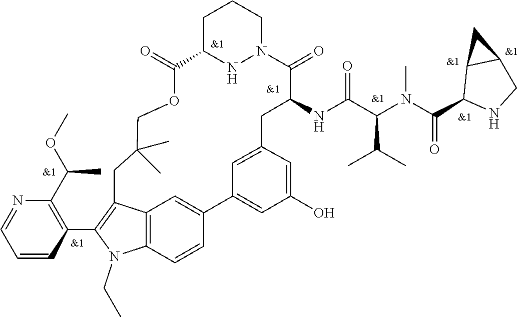 |
| AA218 | 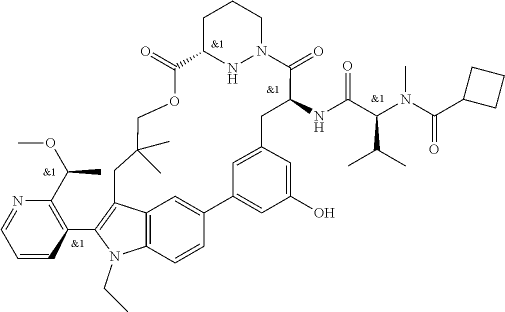 |
| AA219 | 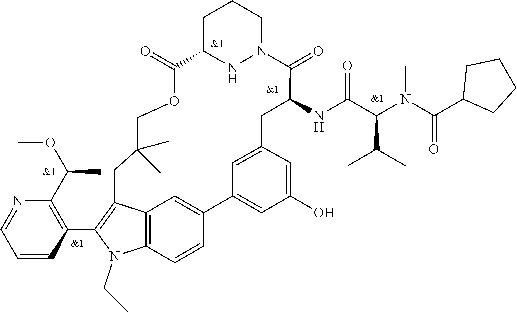 |
| AA220 | 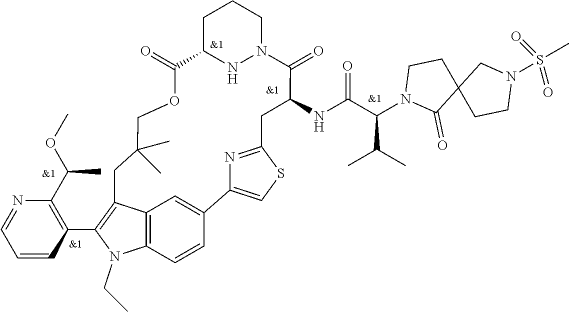 |
| AA221 | 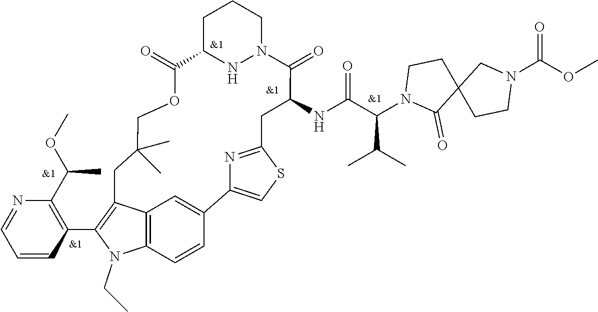 |
| AA222 | 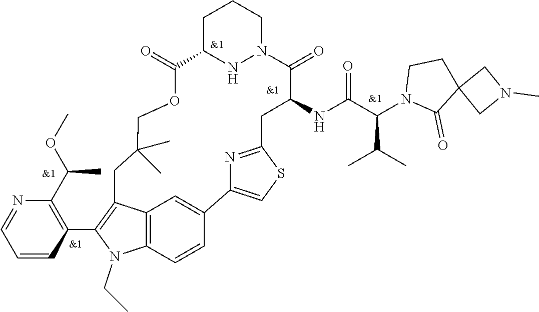 |
| AA223 | 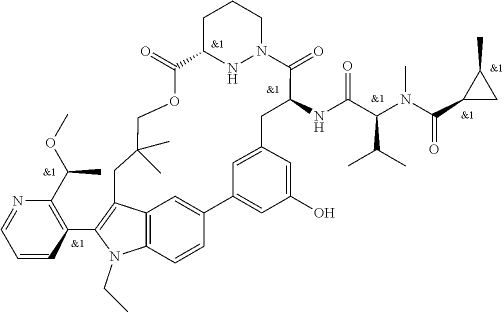 |
| AA224 | 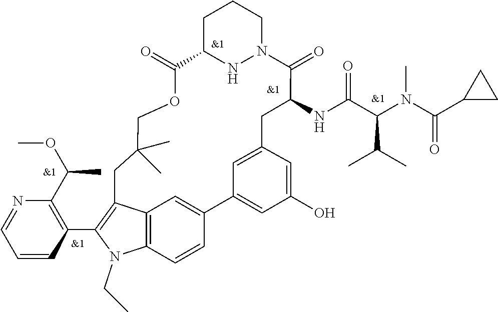 |
| AA225 | 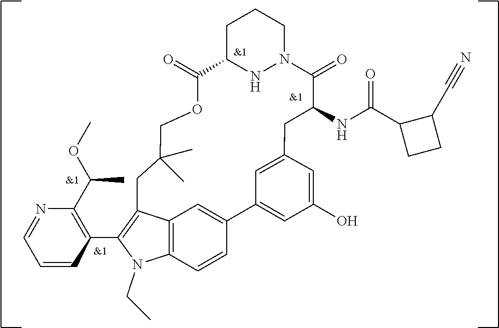 |
| AA226 | 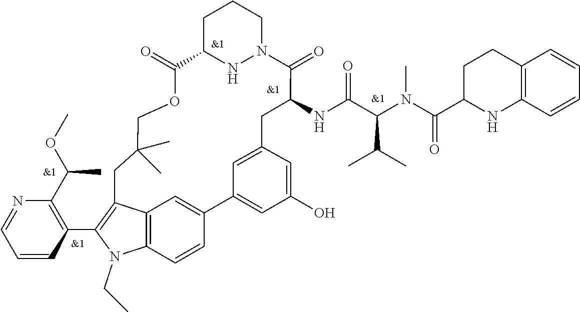 |
| AA227 | 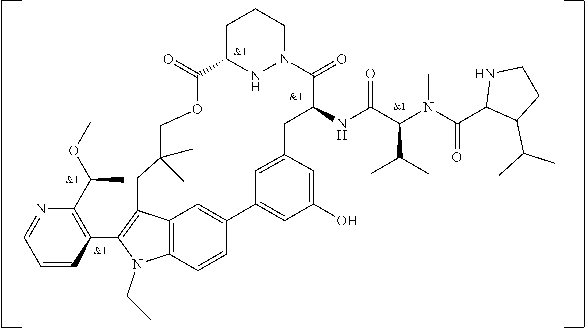 |
| AA228 | 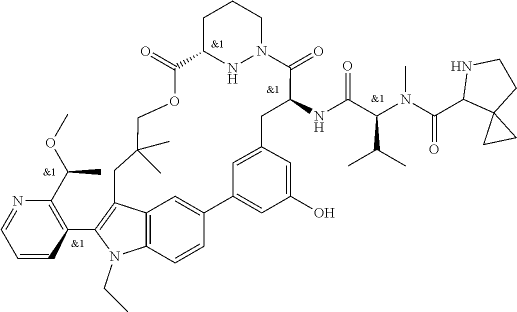 |
| AA229 | 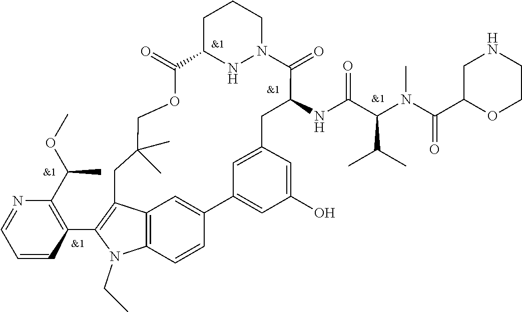 |
| AA230 | 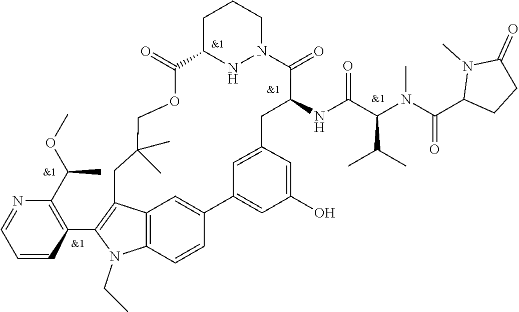 |
| AA231 | 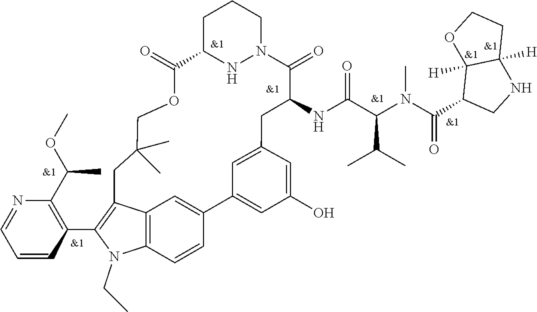 |
| AA232 | 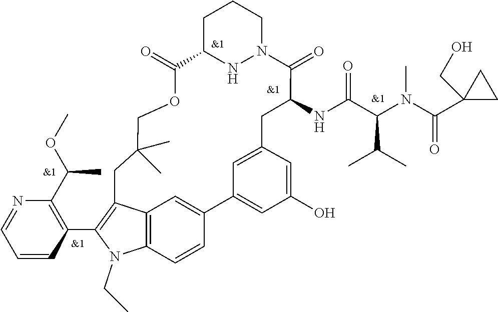 |
| AA233 | 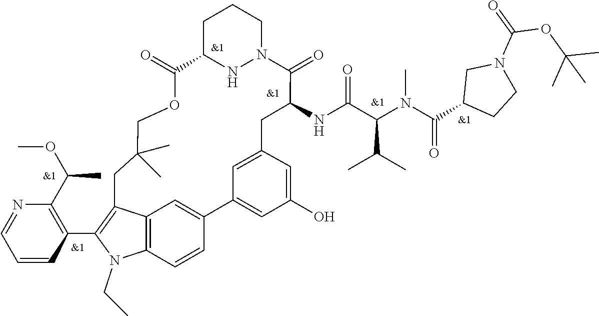 |
| AA234 | 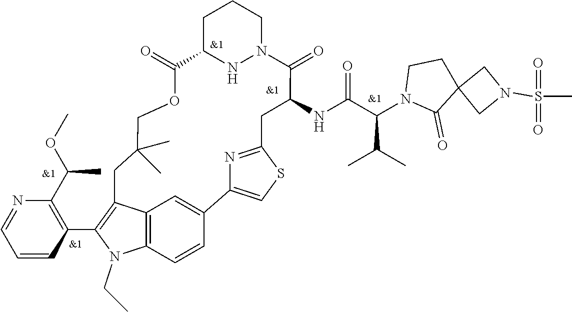 |
| AA235 | 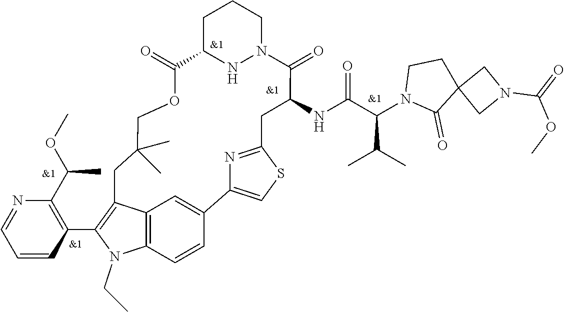 |
| AA236 | 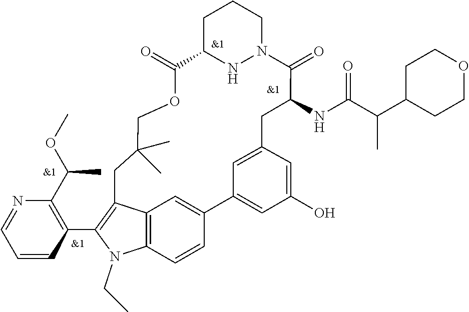 |
| AA237 | 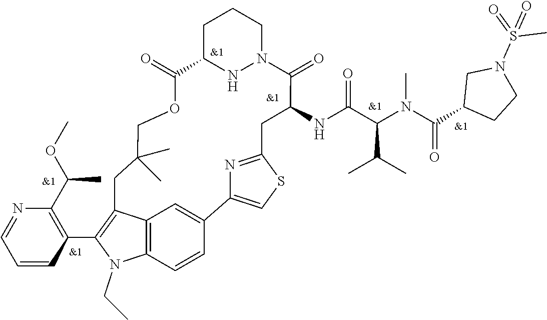 |
| AA238 | 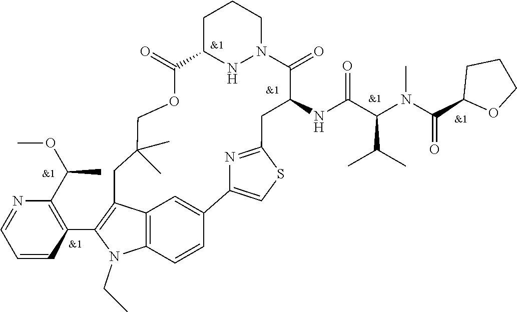 |
| AA239 | 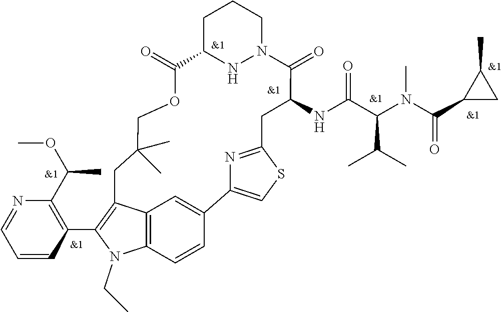 |
| AA240 | 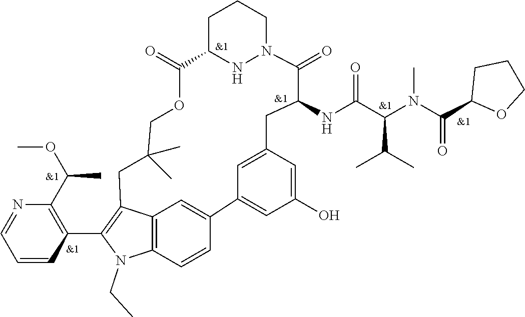 |
| AA241 | 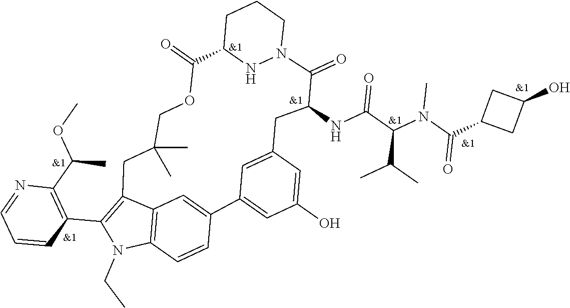 |
| AA242 | 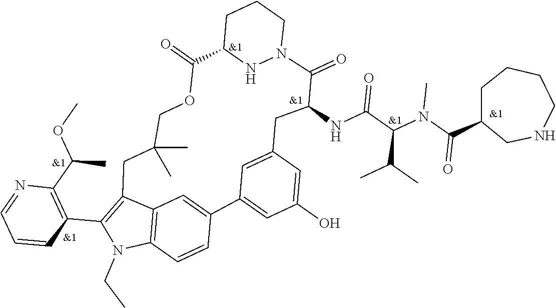 |
| AA243 | 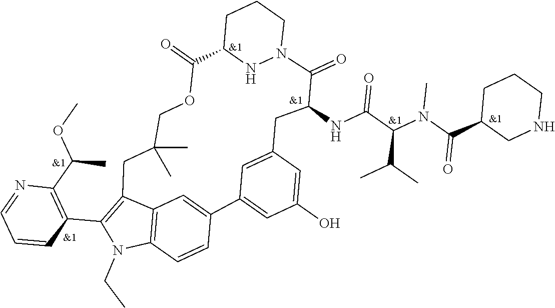 |
| AA244 | 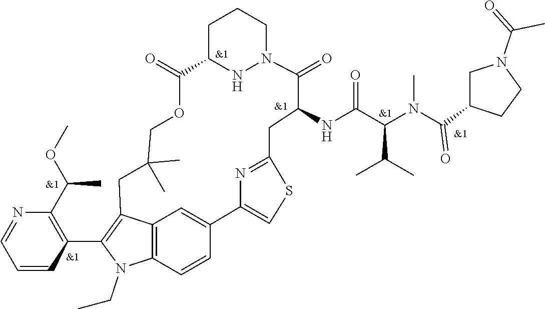 |
| AA245 | 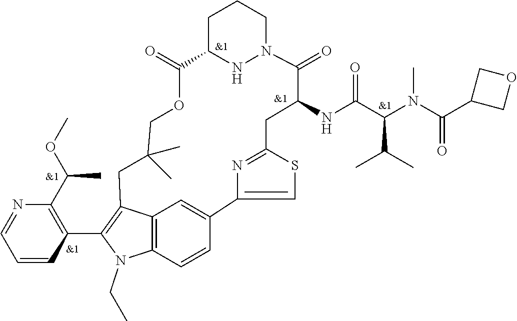 |
| AA246 | 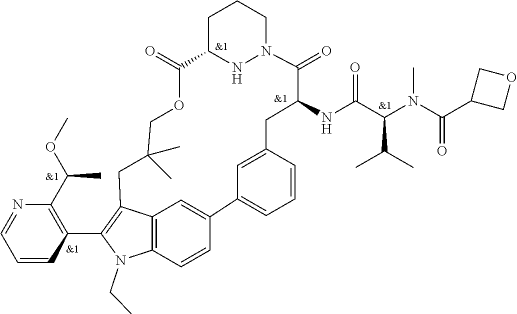 |
| AA247 | 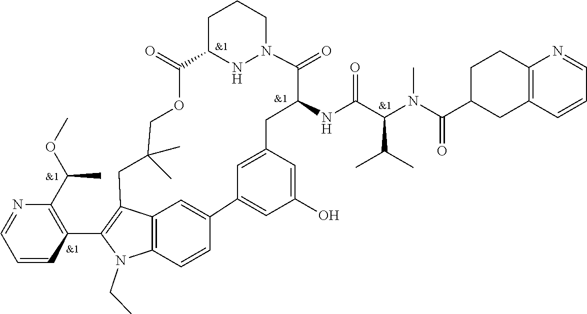 |
| AA248 | 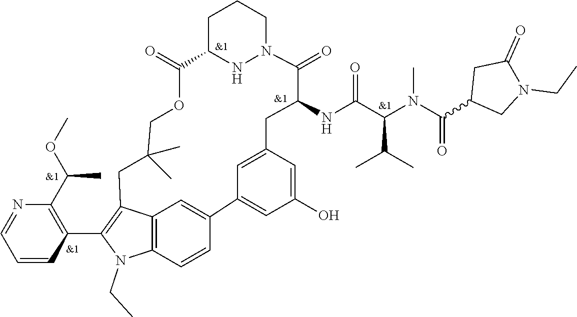 |
| AA249 | 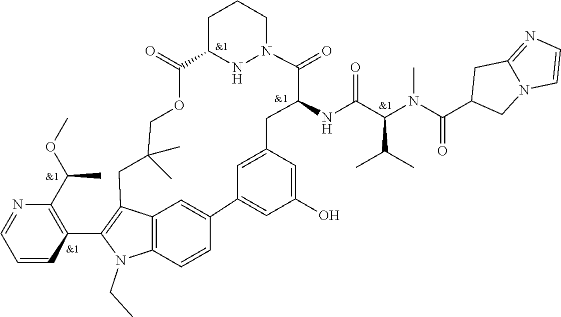 |
| AA250 | 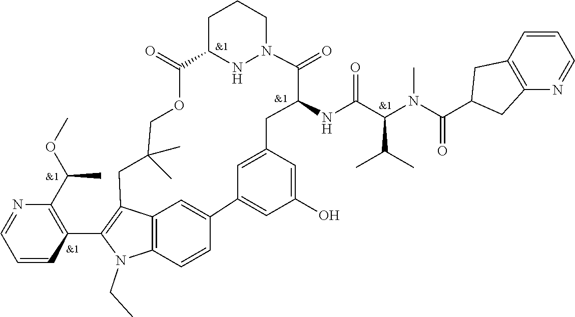 |
| AA251 | 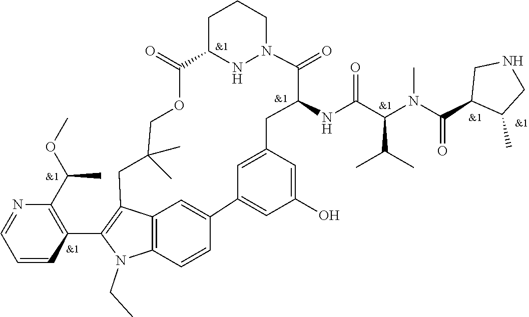 |
| AA252 | 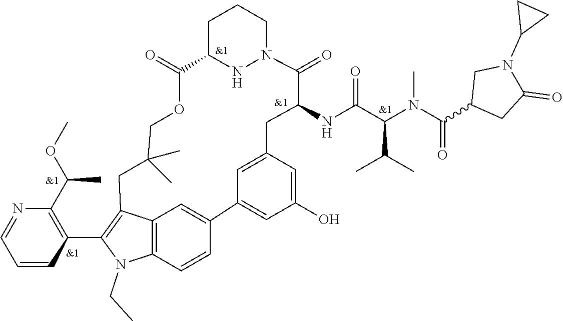 |
| AA253 | 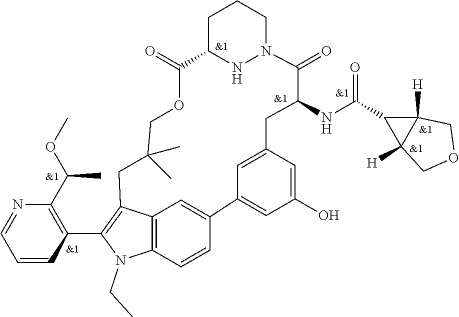 |
| AA254 | 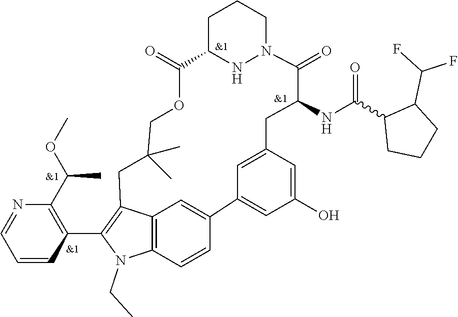 |
| AA255 | 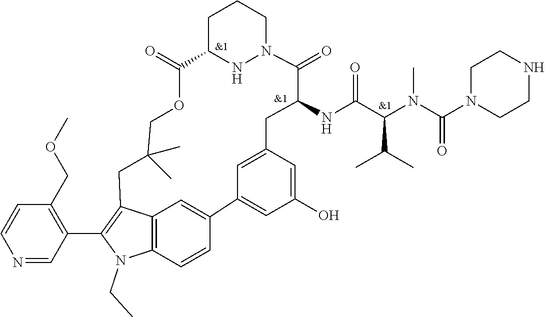 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AA256 | 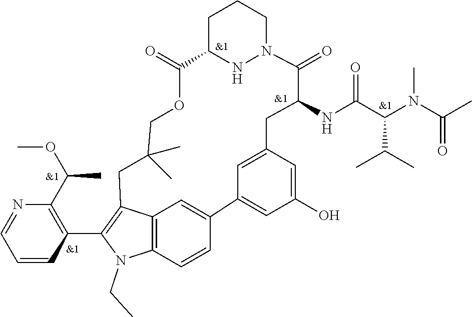 |
| AA257 | 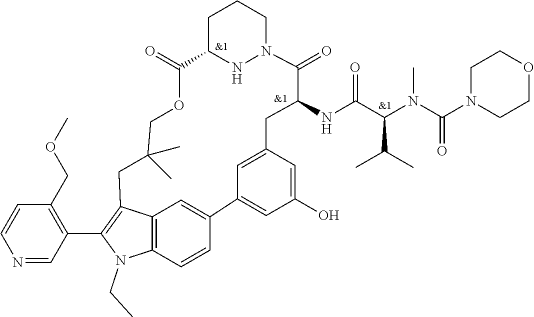 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AA258 | 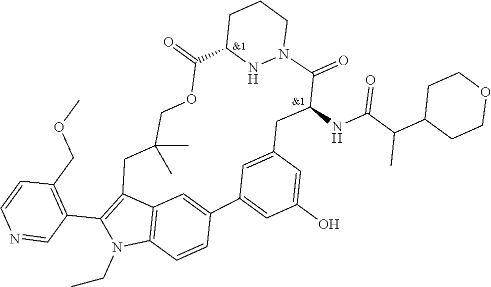 [SMILES: CCn1c(-c2cnccc2COC)c2c3cc(ccc31)-c1cc(O)cc(c1)C[C@H](NC(=O)C(C)C1CCOCC1)C(=O)N1CCC[C@H](N1)C(=O)OCC(C)(C)C2] |
| AA259 | 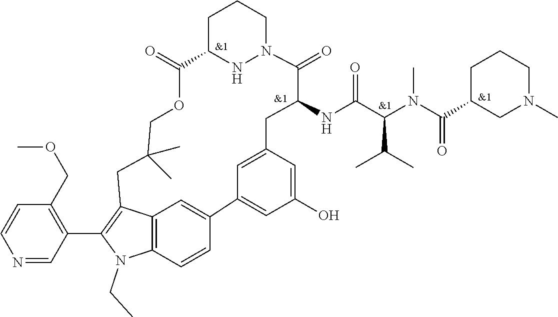 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| AA260 | 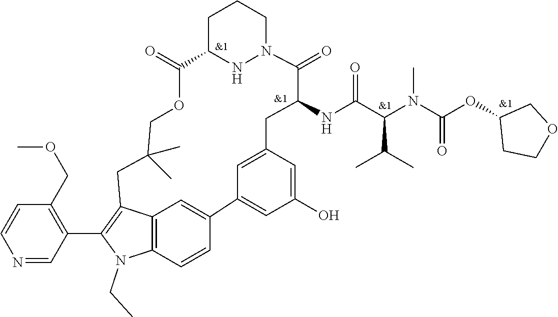 [SMILES: CCn1c(-c2cnccc2COC)c2c3cc(ccc31)-c1cc(O)cc(c1)C[C@H](NC(=O)[C@H](C(C)C)N(C)C(=O)O[C@H]1CCOC1)C(=O)N1CCC[C@H](N1)C(=O)OCC(C)(C)C2] |
| AA261 | 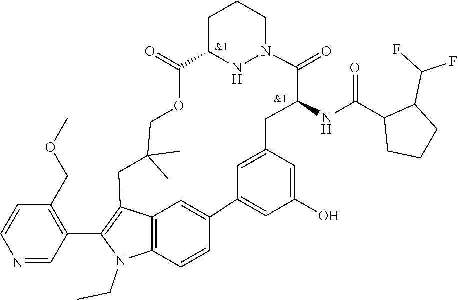 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| AA262 | 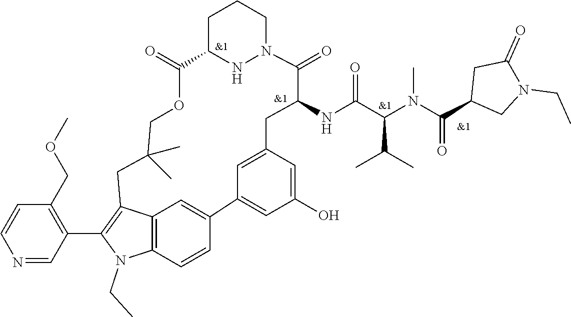 [SMILES: CCN1C[C@@H](C(=O)N(C)[C@H](C(=O)N[C@H]2Cc3cc(O)cc(c3)-c3ccc4c(c3)c(c(-c3cnccc3COC)n4CC)CC(C)(C)COC(=O)[C@@H]3CCCN(N3)C2=O)C(C)C)CC1=O] |
| AA263 | 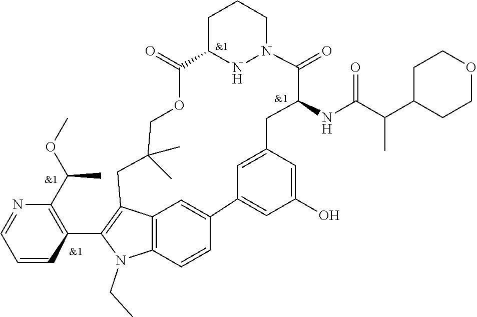 |
| AA264 | 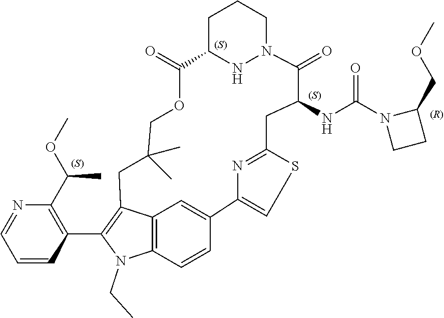 |
| AA265 | 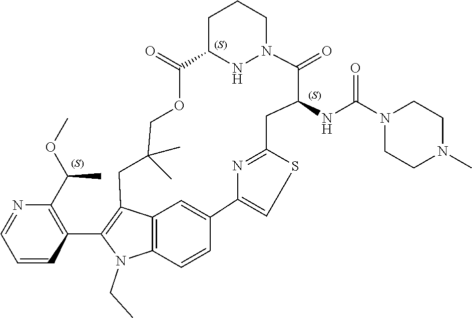 |
| AA266 | 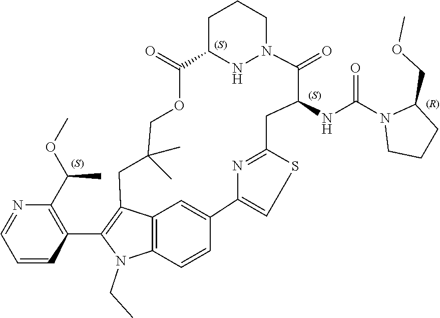 |
| AA267 | 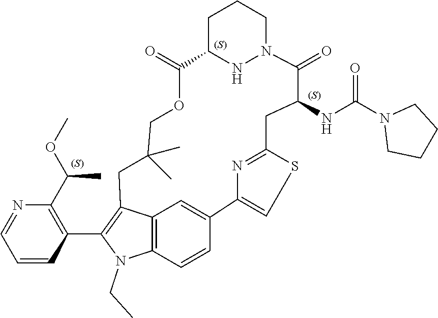 |
| AA268 | 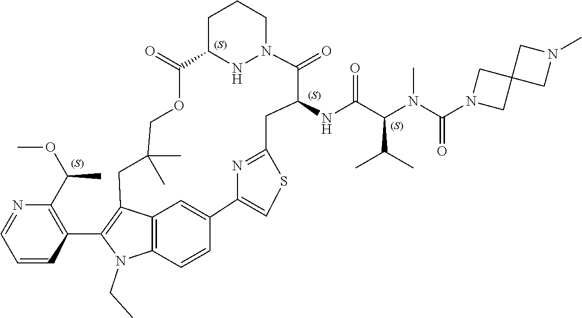 |
| AA270 | 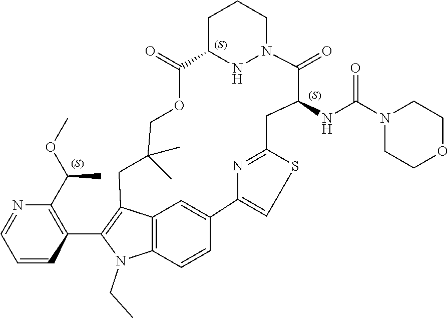 |
| AA271 | 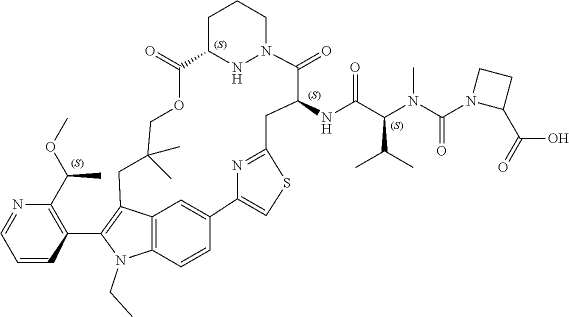 |
| AA272 | 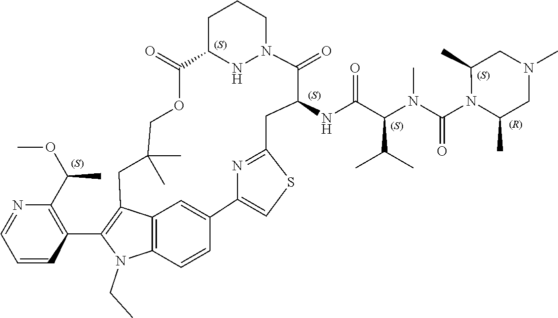 |
| AA273 | 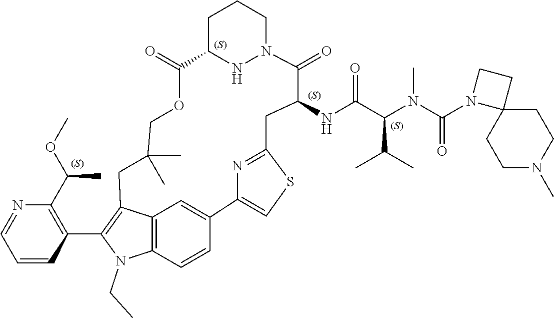 |
| AA274 | 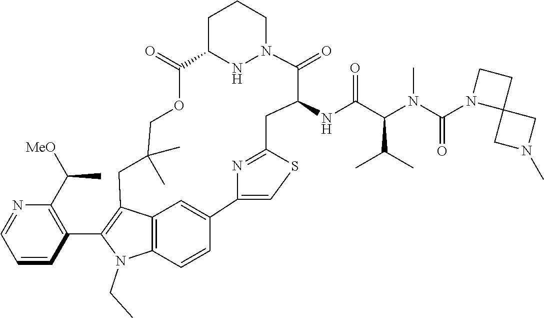 |
| AA275 | 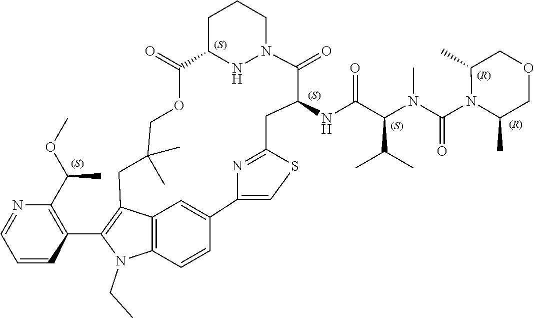 |
| AA276 | 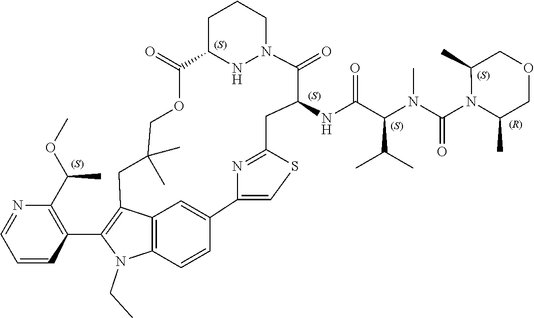 |
| AA277 | 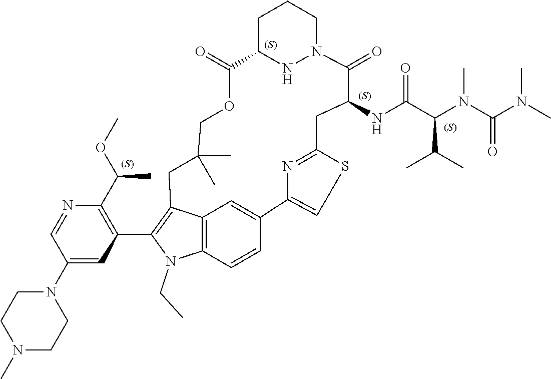 |
| AA278 | 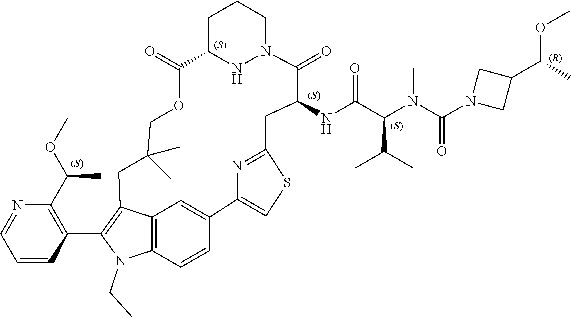 |
| AA279 | 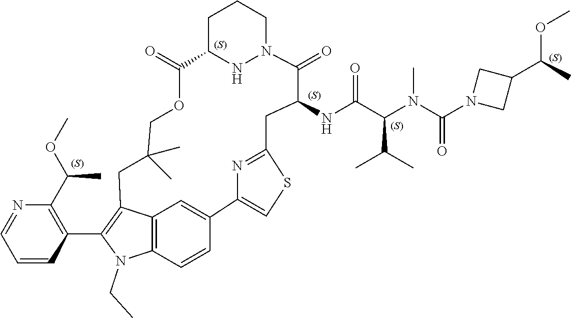 |
| AA280 | 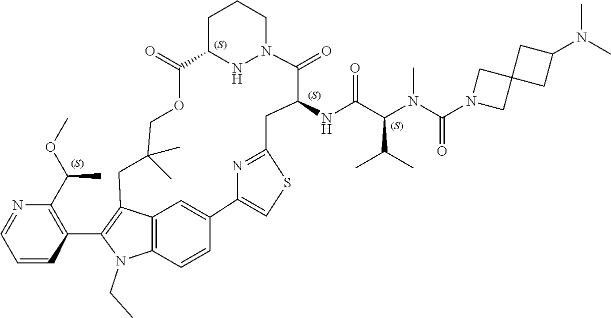 |
| AA281 | 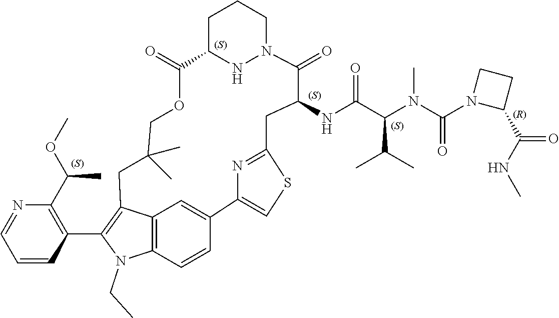 |
| AA282 | 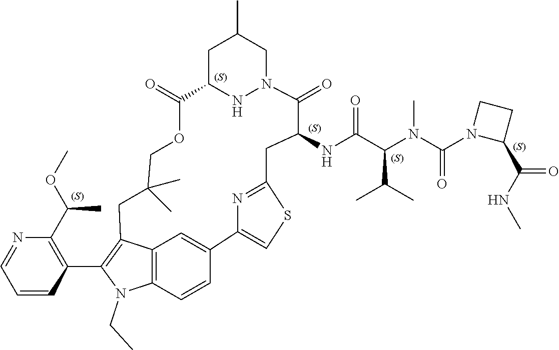 |
| AA283 | 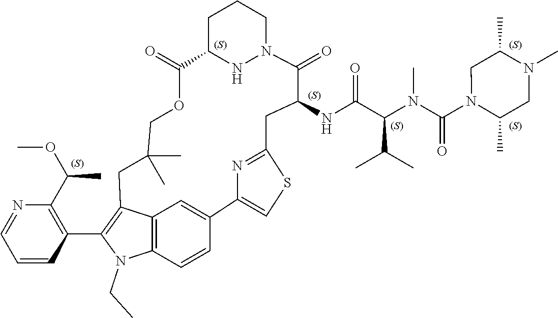 |
| AA284 | 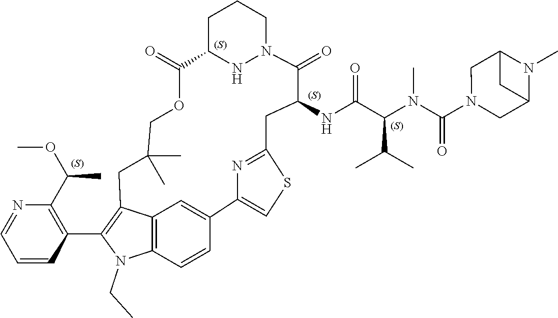 |
| AA285 | 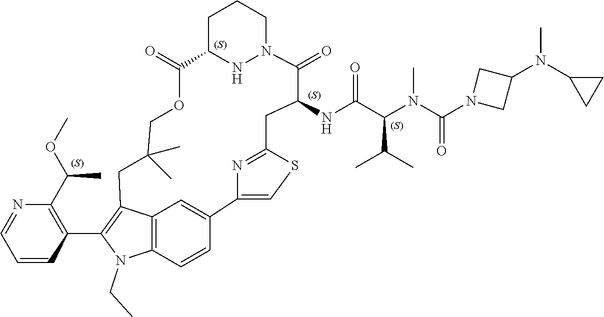 |
| AA286 | 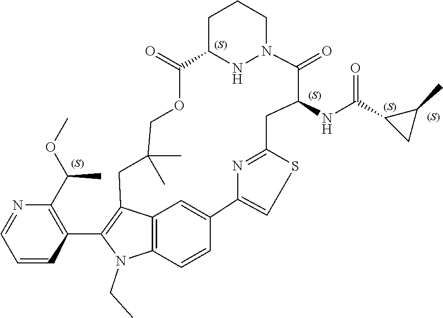 |
| AA287 | 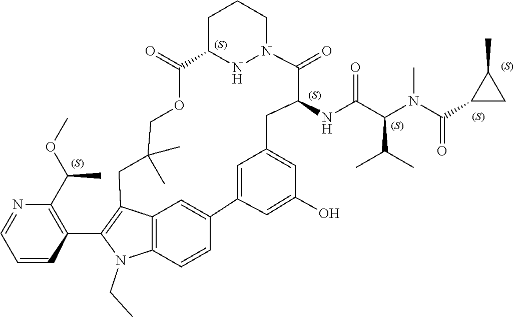 |
| AA288 | 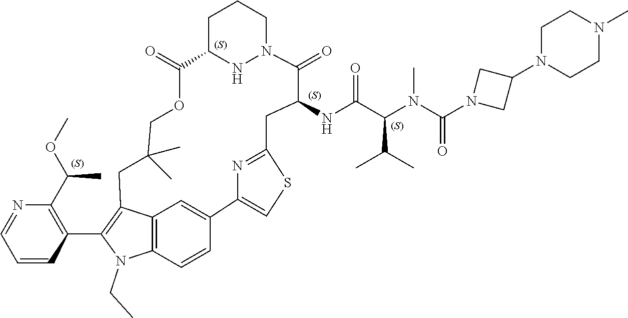 |
| AA289 | 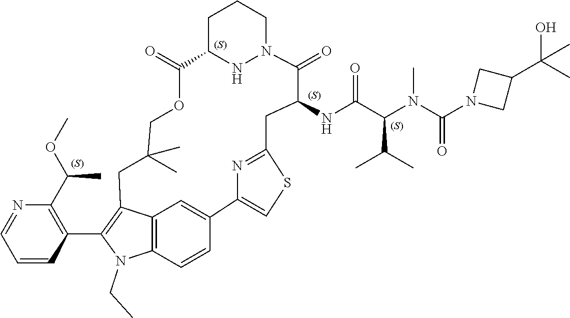 |
| AA290 | 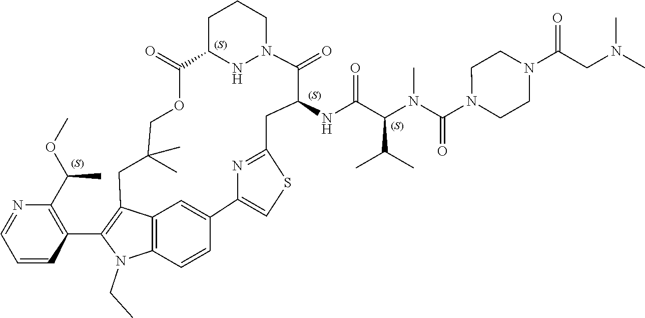 |
| AA291 | 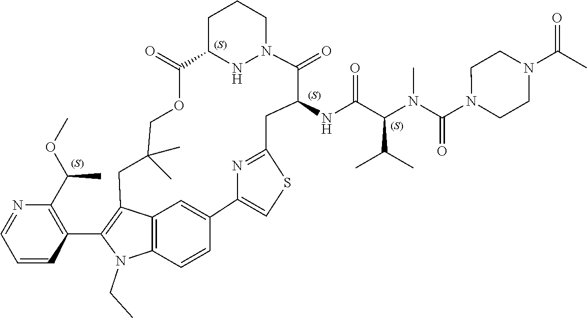 |
| AA292 | 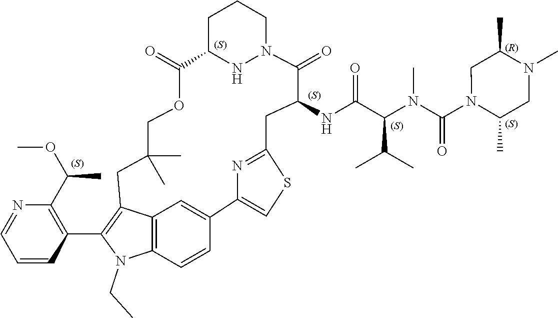 |
| AA293 | 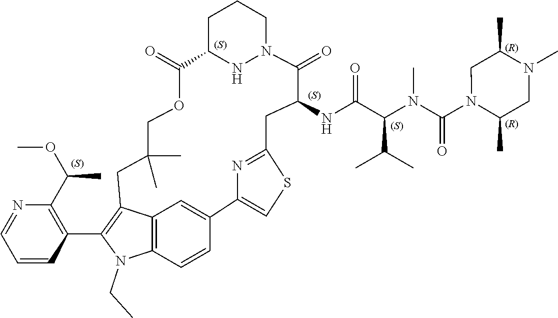 |
| AA294 | 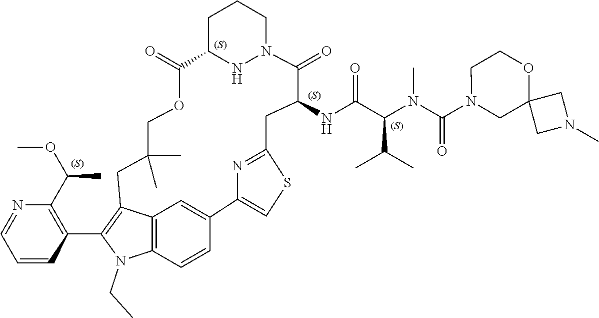 |
| AA295 | 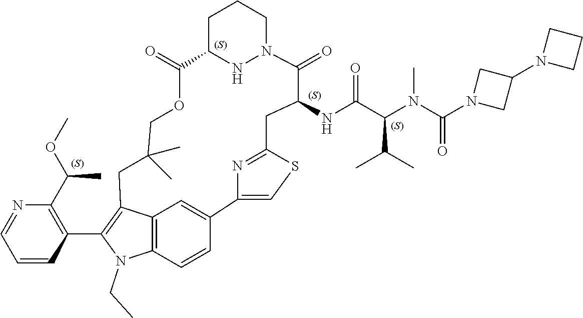 |
| AA296 | 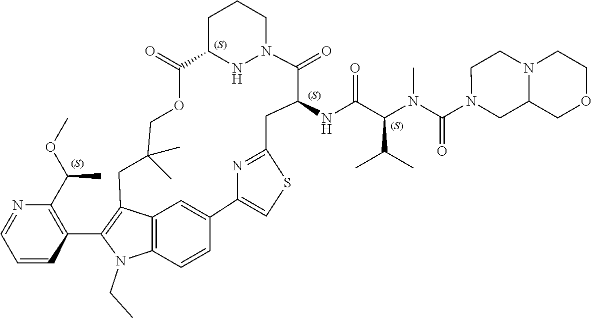 |
| AA297 | 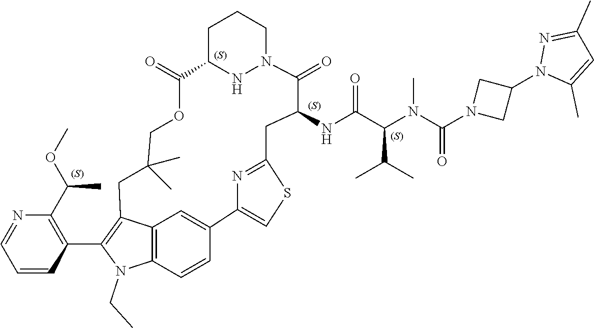 |
| AA298 | 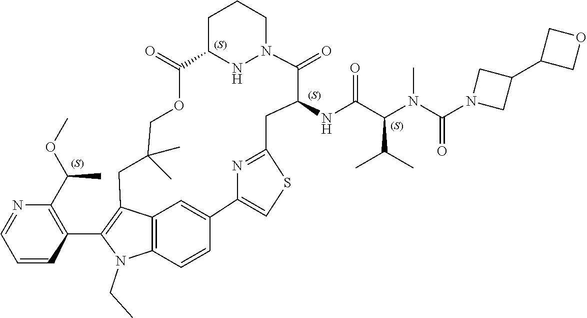 |
| AA299 | 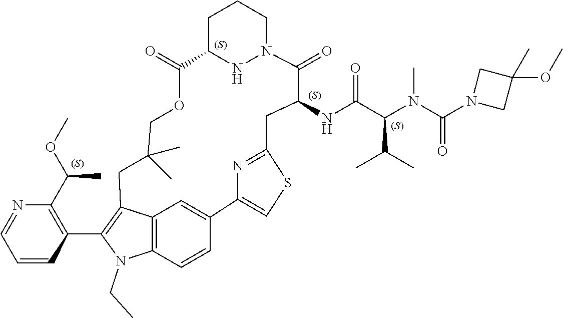 |
| AA300 | 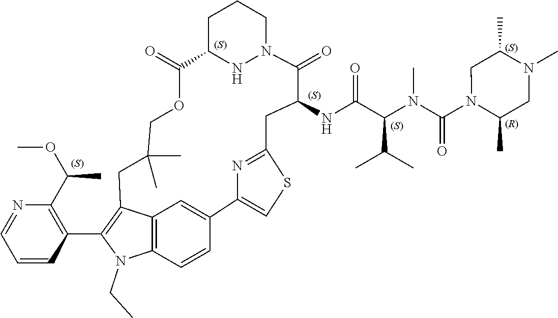 |
| AA301 | 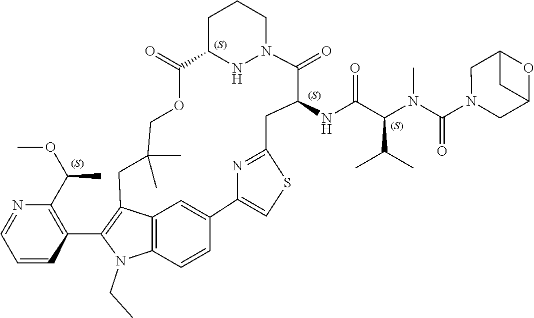 |
| AA302 | 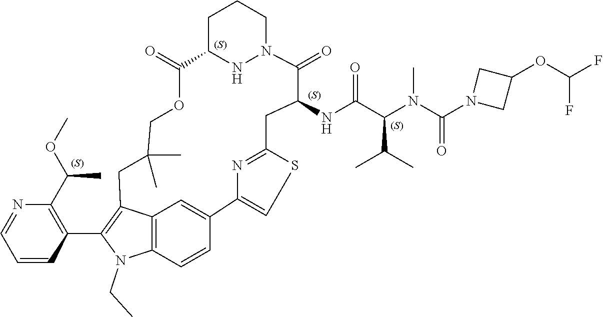 |
| AA303 | 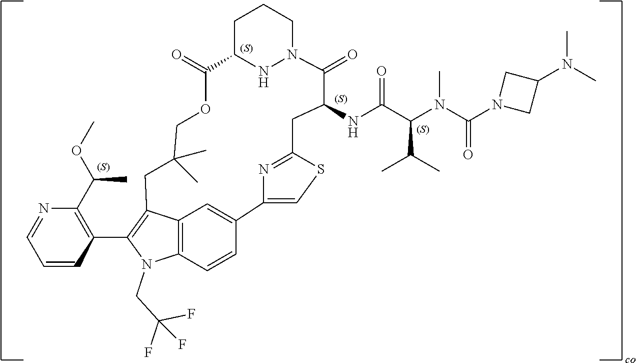 |
| AA304 | 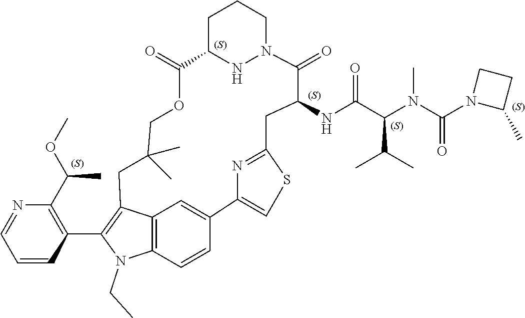 |
| AA305 | 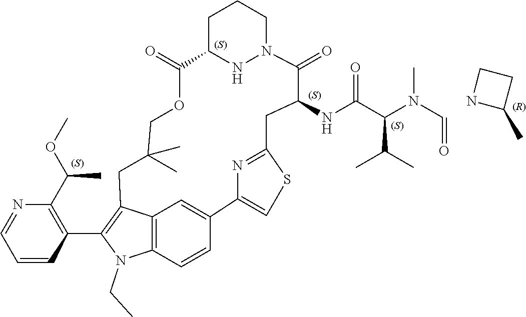 |
| AA306 | 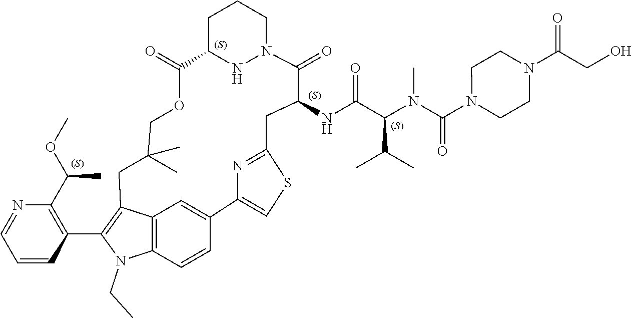 |
| AA307 | 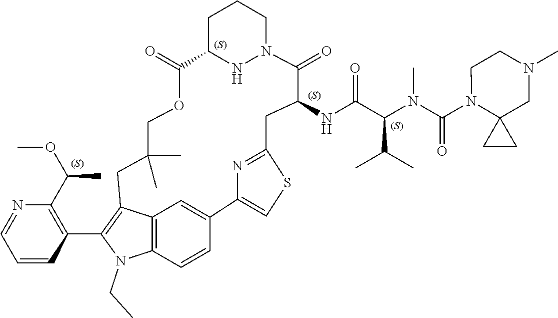 |
| AA308 | 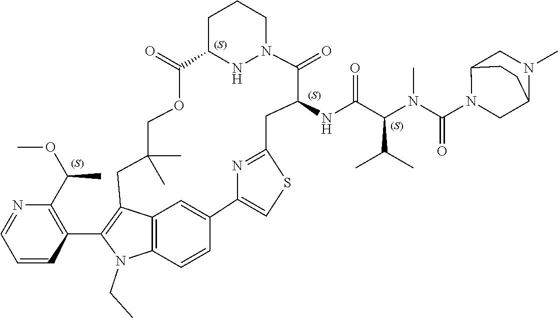 |
| AA309 | 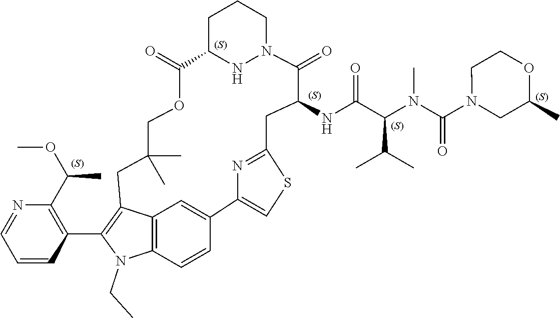 |
| AA310 | 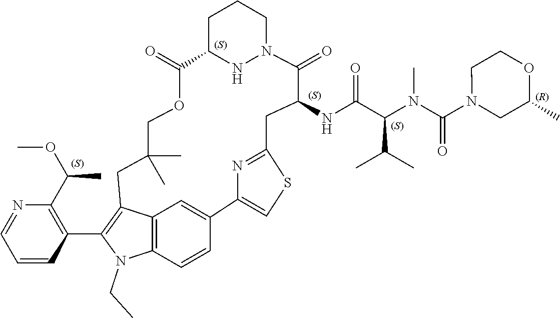 |
| AA311 | 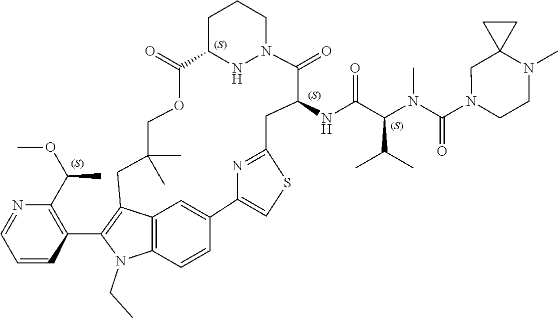 |
| AA312 | 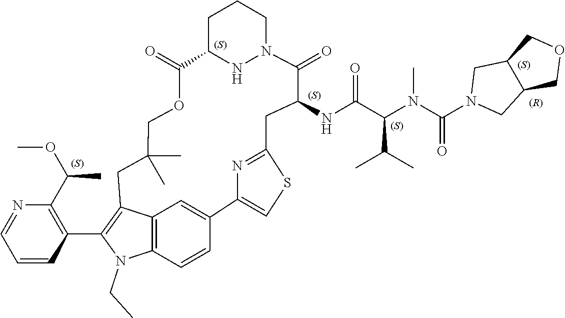 |
| AA313 | 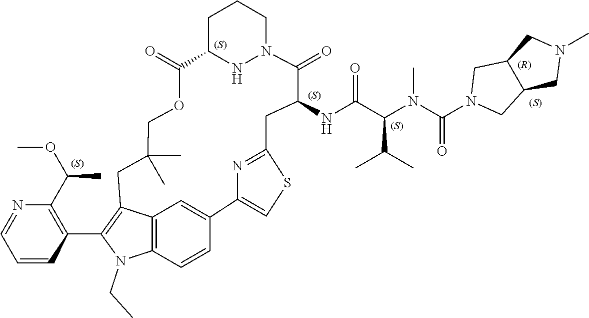 |
| AA314 | 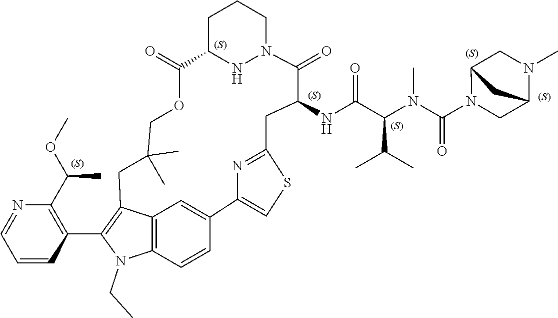 |
| AA315 | 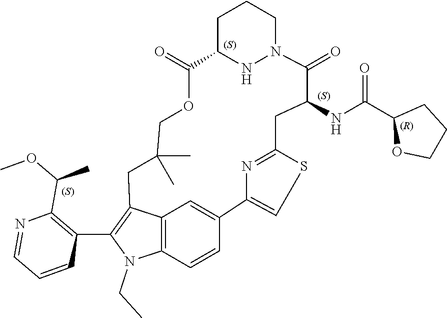 |
| AA316 | 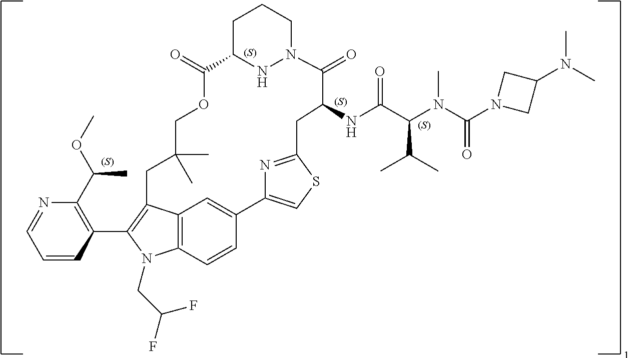 |
| AA317 | 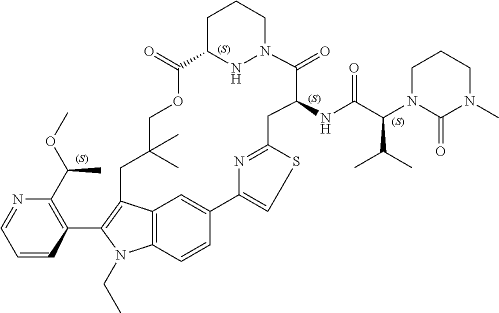 |
| AA318 | 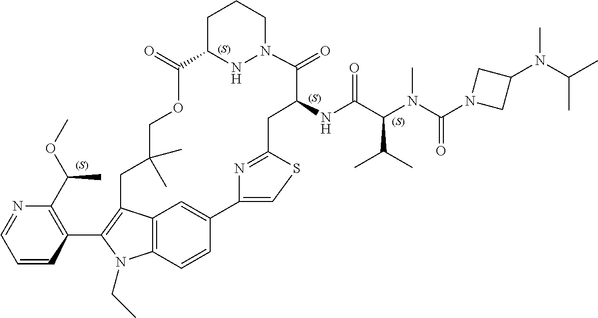 |
| AA319 | 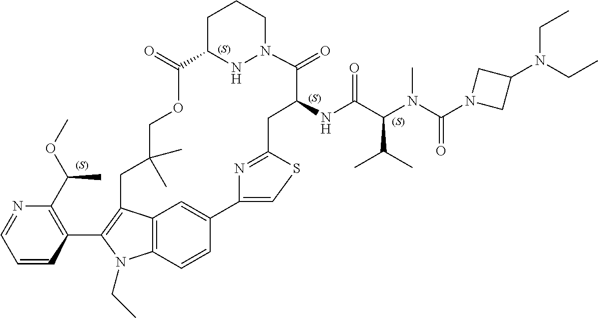 |
| AA320 | 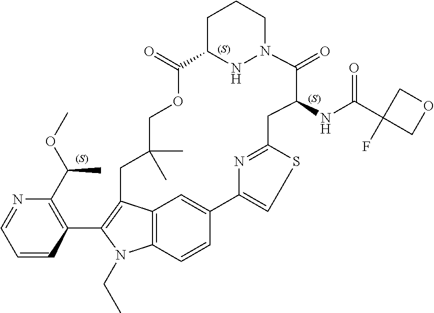 |
| AA321 | 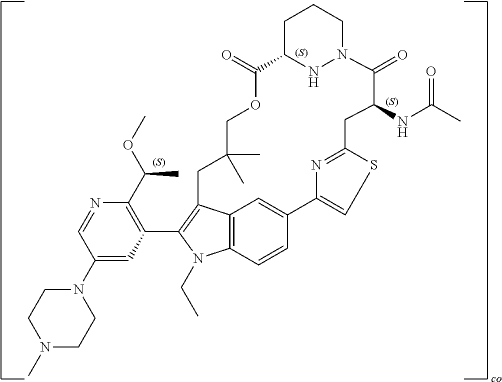 |
| AA322 | 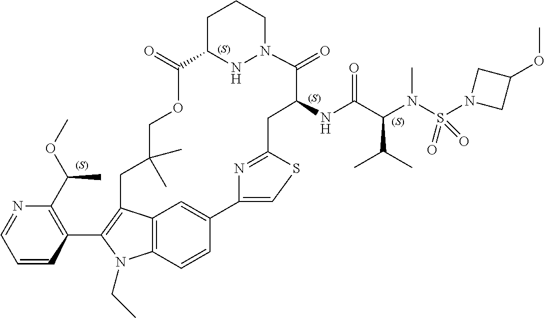 |
| AA323 | 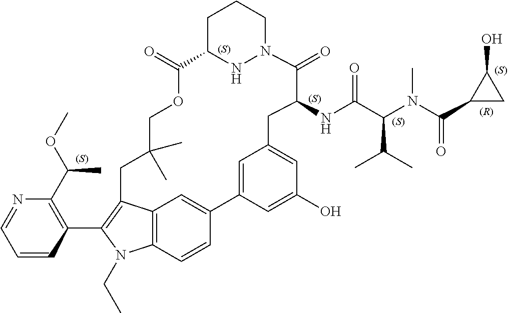 |
| AA324 | 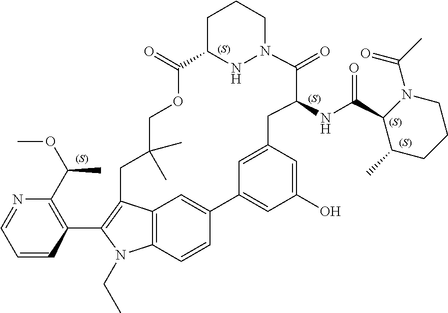 |
| AA325 | 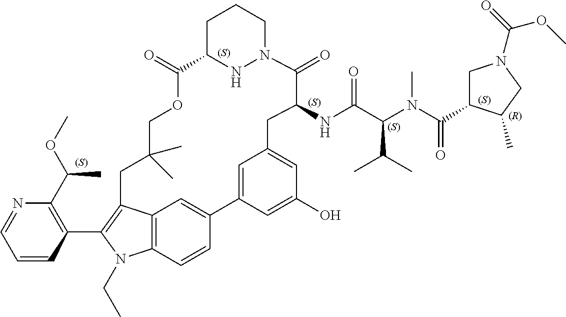 |
| AA326 | 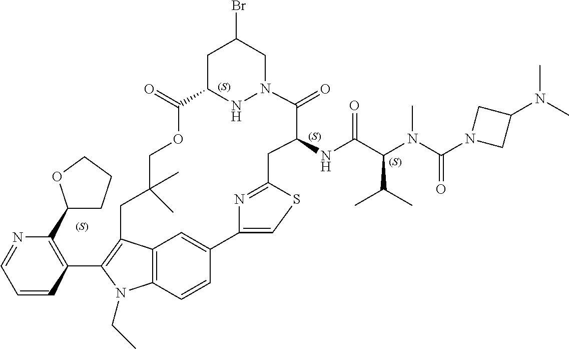 |
| AA327 | 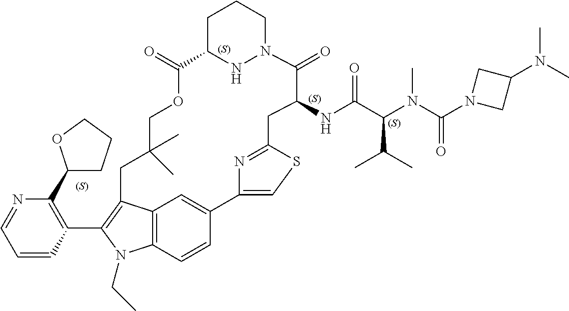 |
| AA328 | 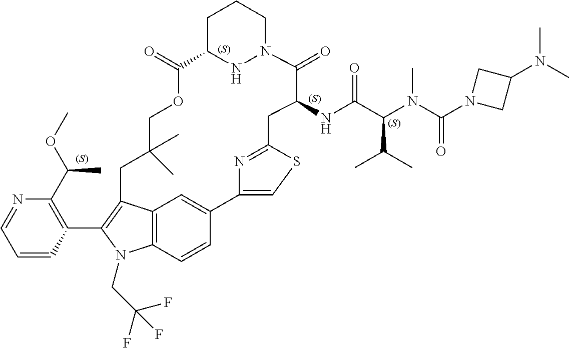 |
| AA329 | 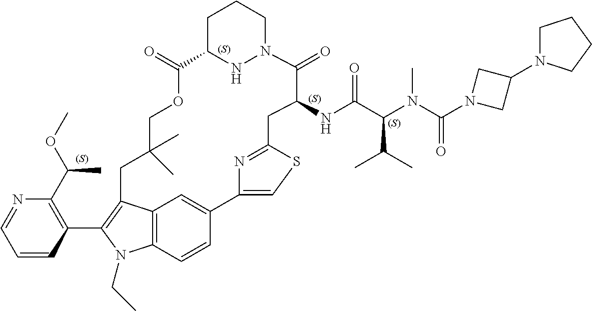 |
| AA330 | 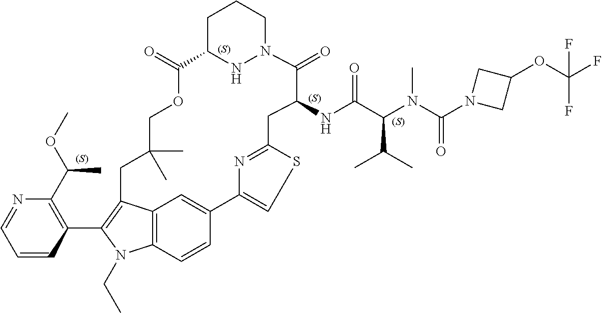 |
| AA331 | 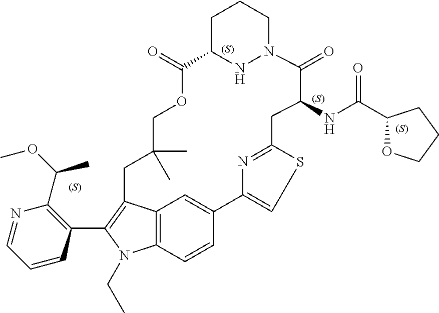 |
| AA332 | 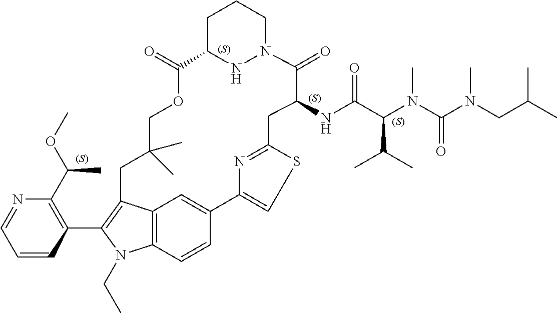 |
| AA333 | 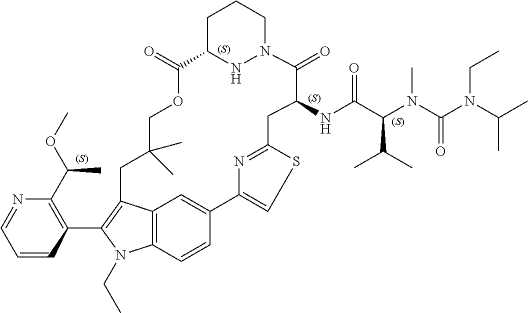 |
| AA334 | 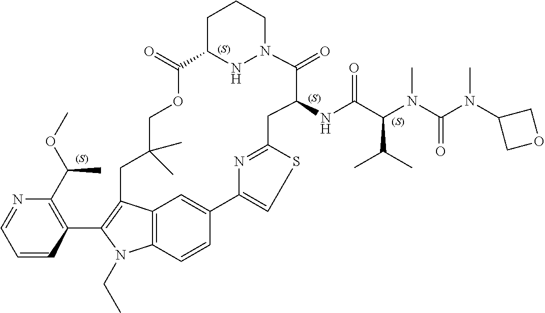 |
| AA335 | 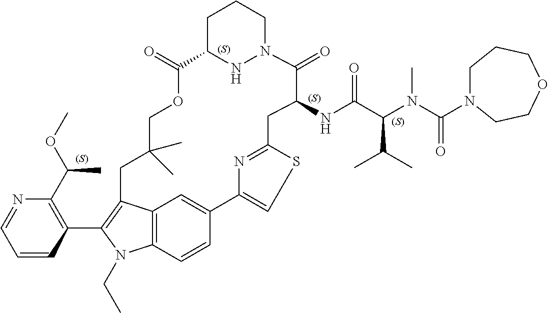 |
| AA336 | 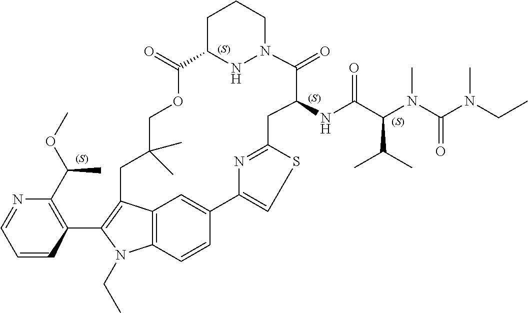 |
| AA337 | 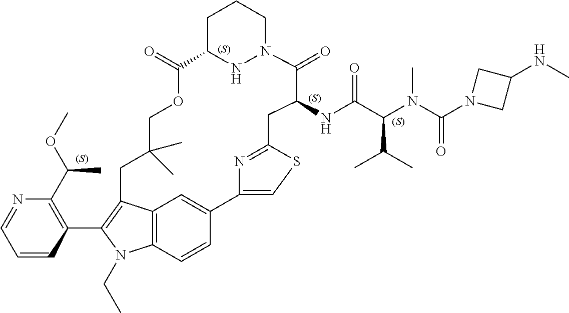 |
| AA338 | 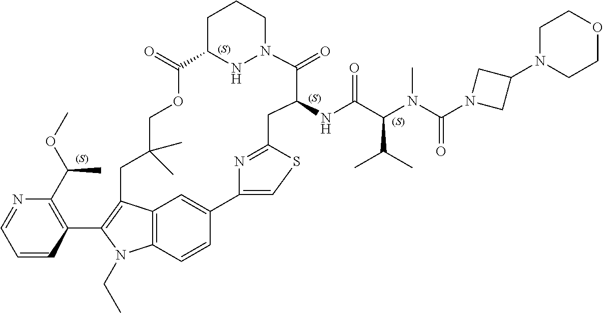 |
| AA339 | 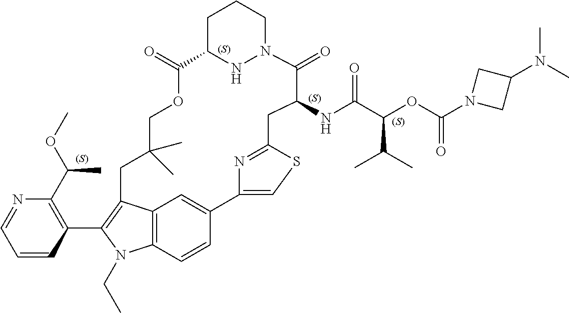 |
| AA340 | 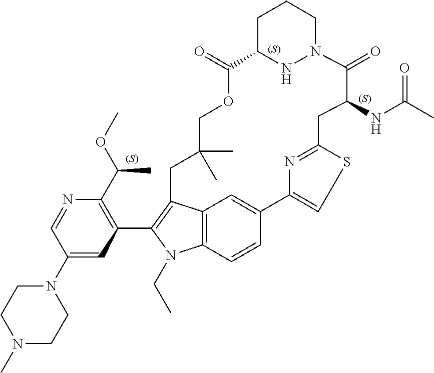 |
| AA341 | 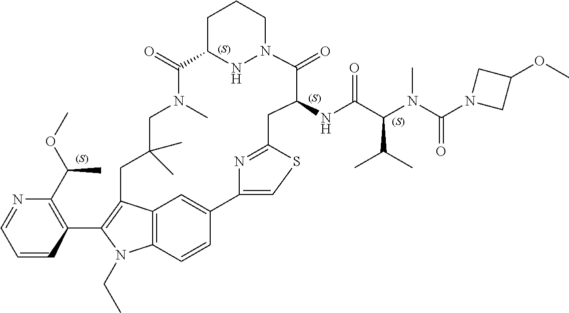 |
| AA342 | 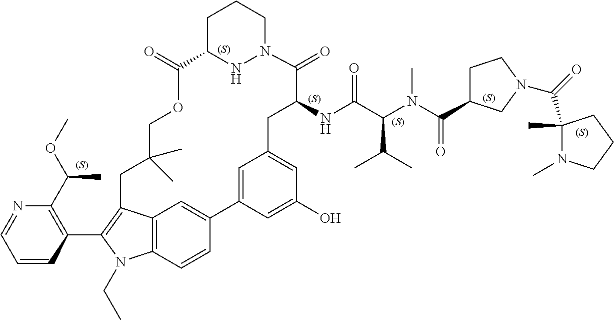 |
| AA343 | 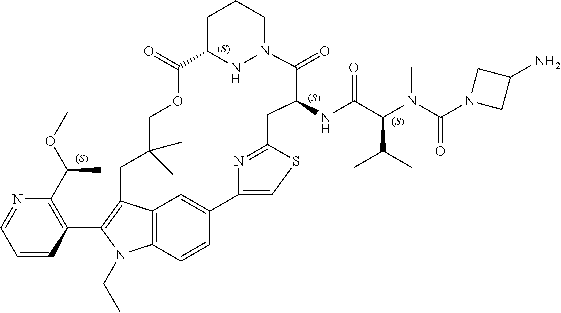 |
| AA344 | 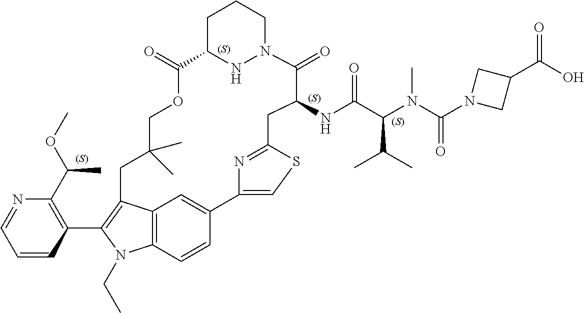 |
| AA345 | 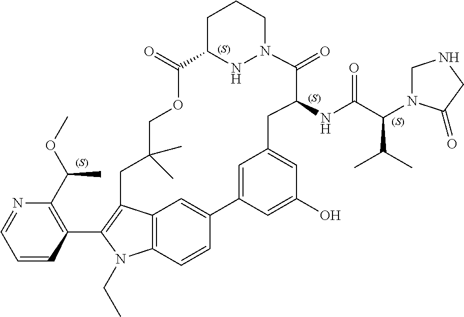 |
| AA346 | 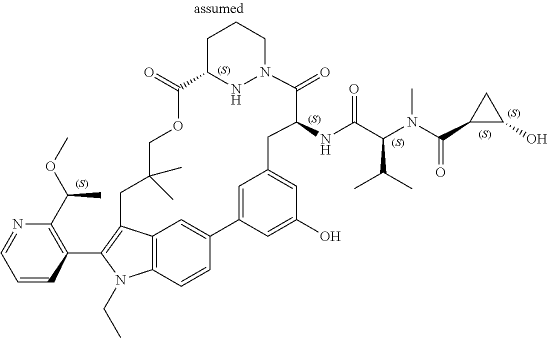 |
| AA347 | 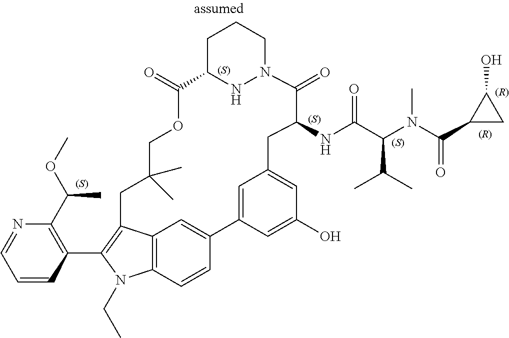 |
| AA348 | 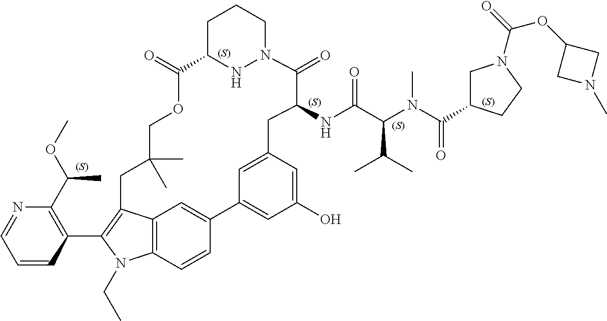 |
| AA349 | 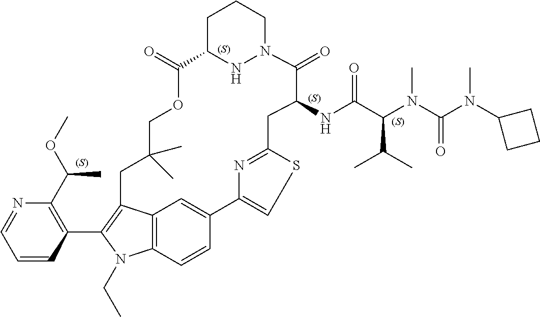 |
| AA350 | 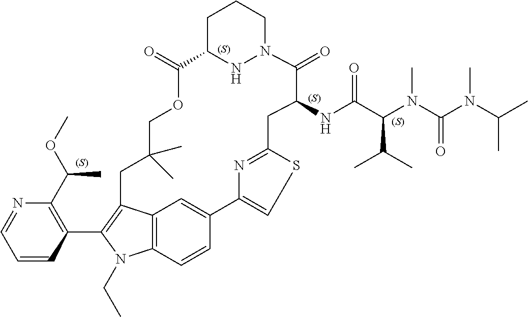 |
| AA351 | 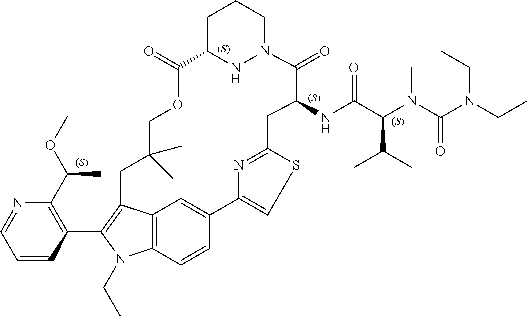 |
| AA352 | 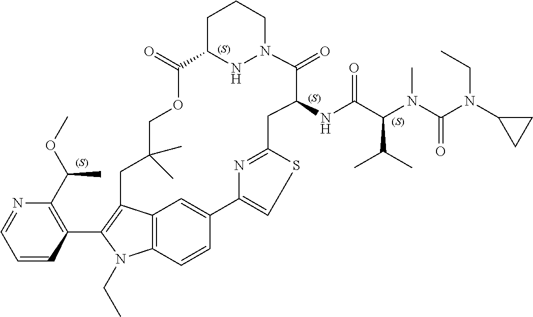 |
| AA353 | 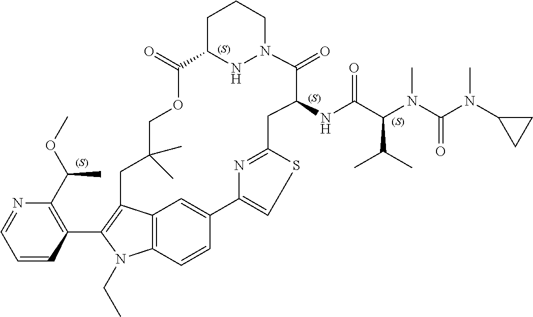 |
| AA354 | 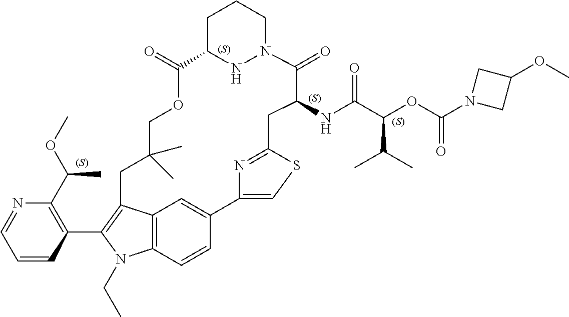 |
| AA355 | 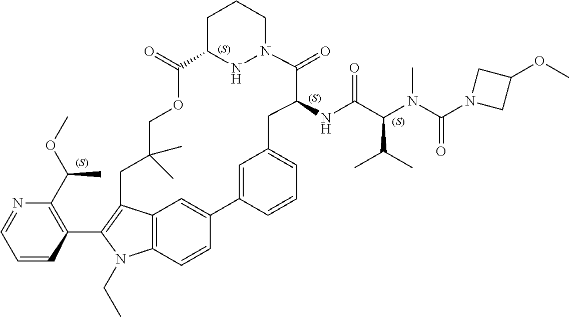 |
| AA356 | 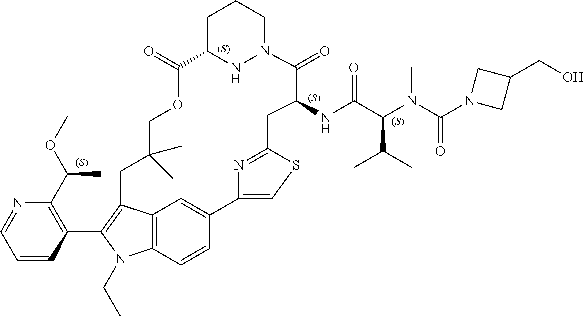 |
| AA357 | 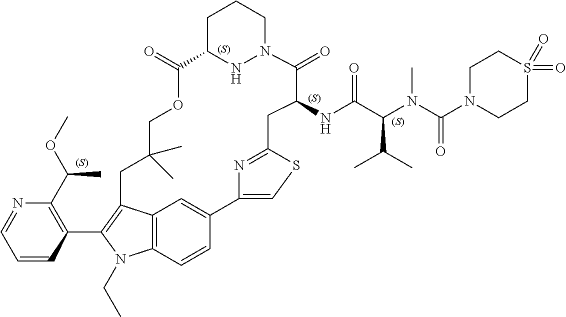 |
| AA358 | 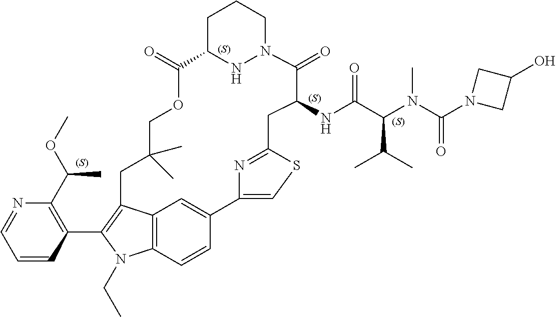 |
| AA359 | 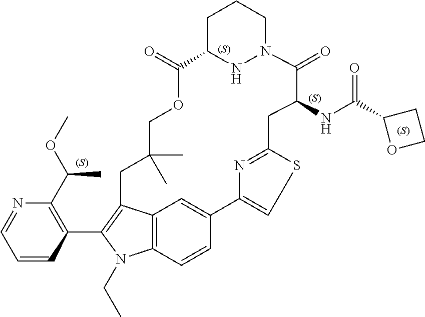 |
| AA360 | 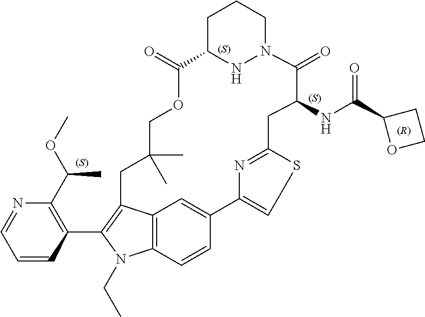 |
| AA361 | 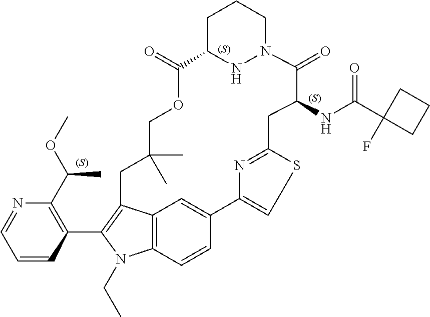 |
| AA362 | 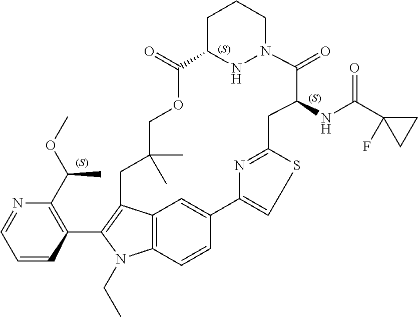 |
| AA363 | 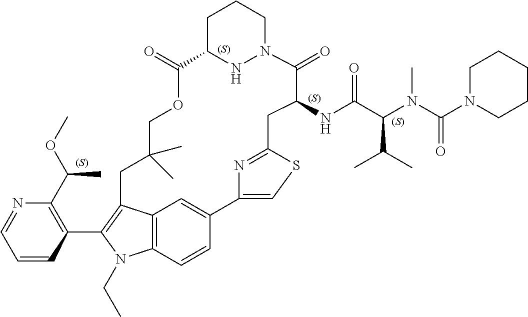 |
| AA364 | 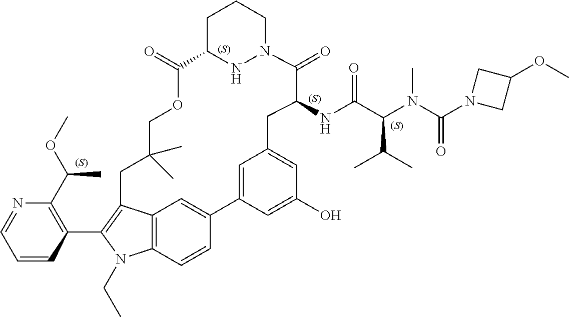 |
| AA365 | 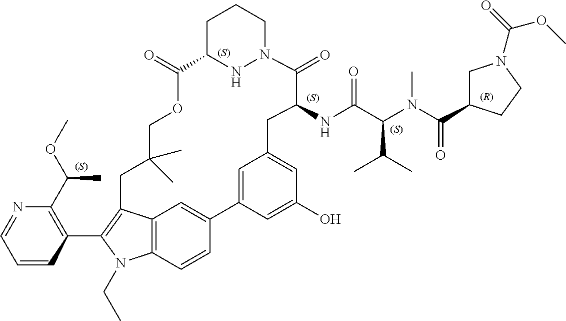 |
| AA366 | 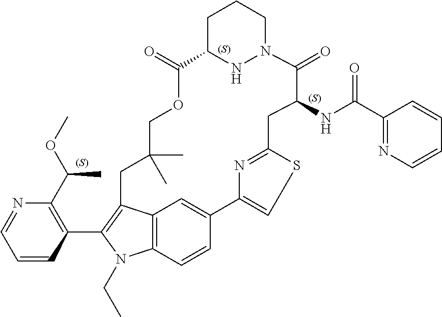 |
| AA367 | 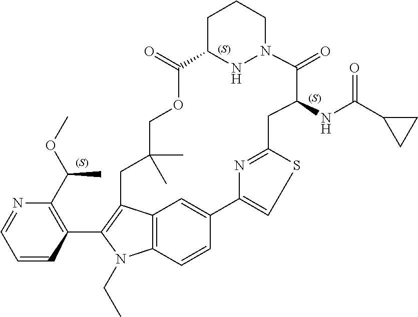 |
| AA368 | 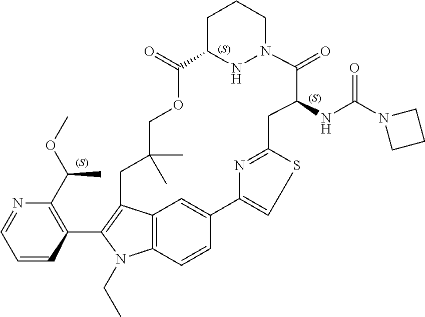 |
| AA369 | 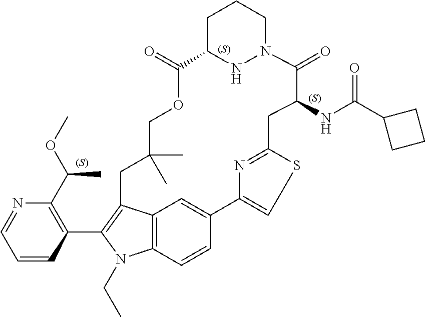 |
| AA370 | 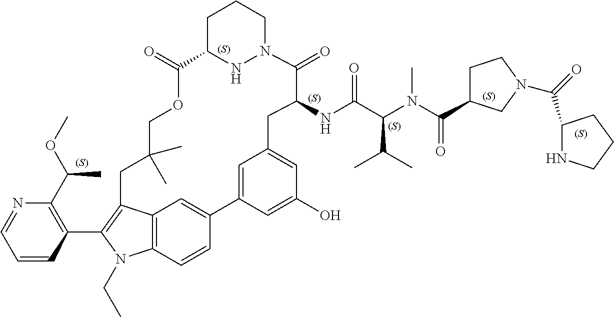 |
| AA371 | 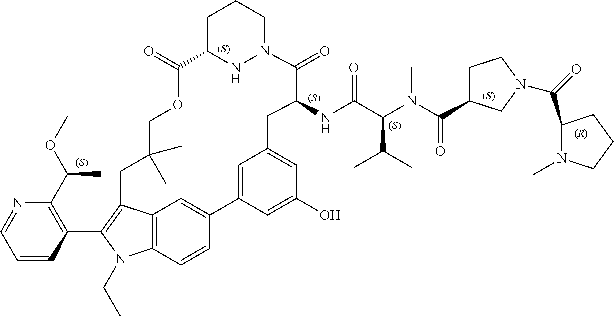 |
| AA372 | 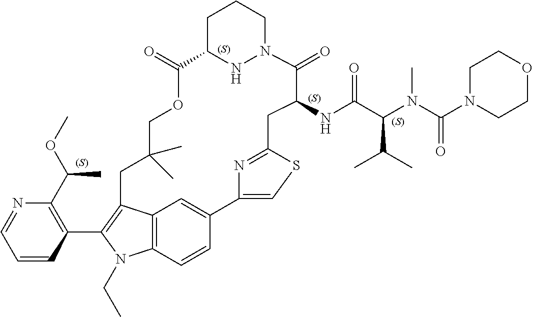 |
| AA373 | 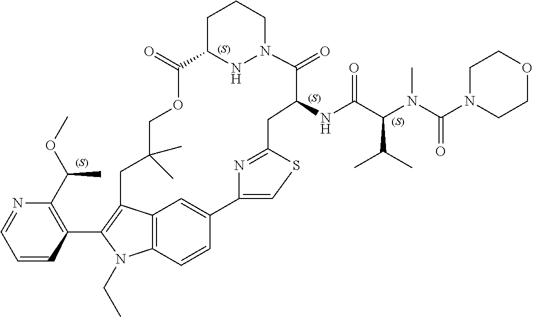 |
| AA374 | 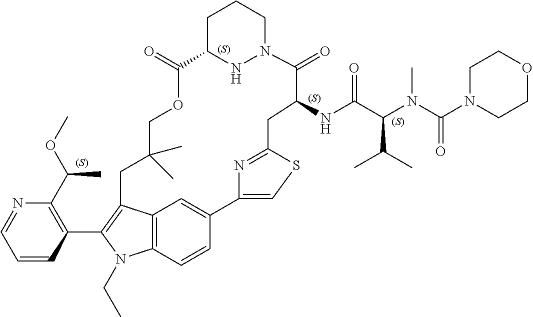 |
| AA375 | 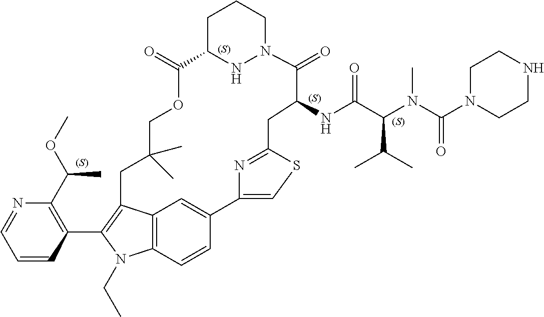 |
| AA376 | 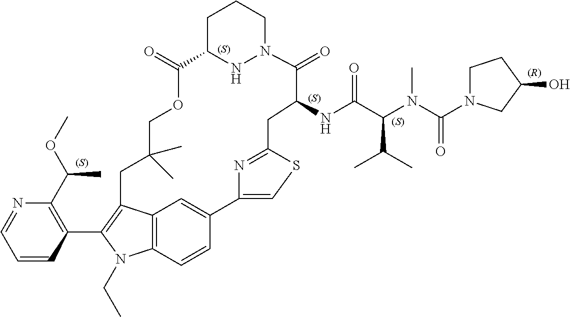 |
| AA377 | 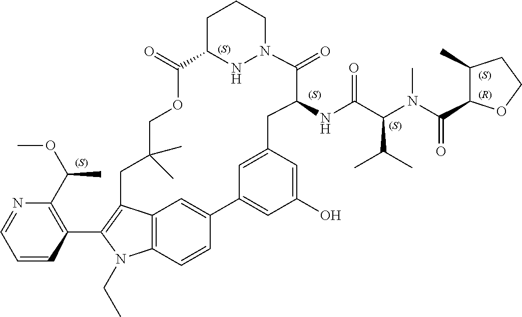 |
| AA378 | 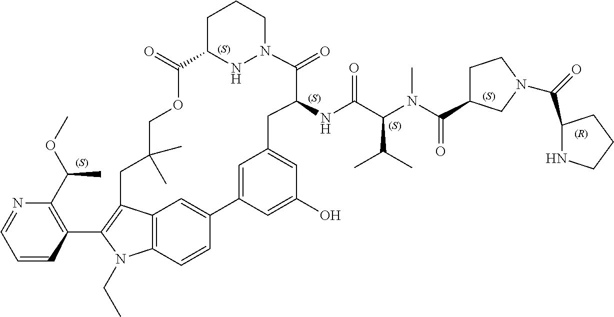 |
| AA379 | 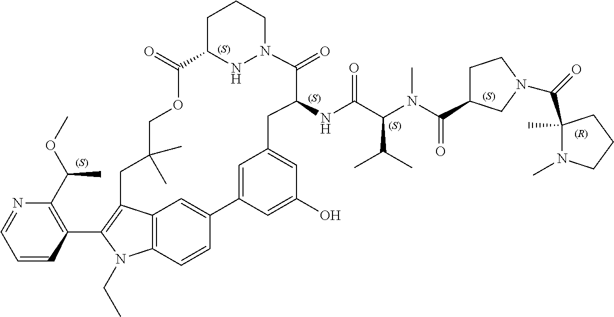 |
| AA380 | 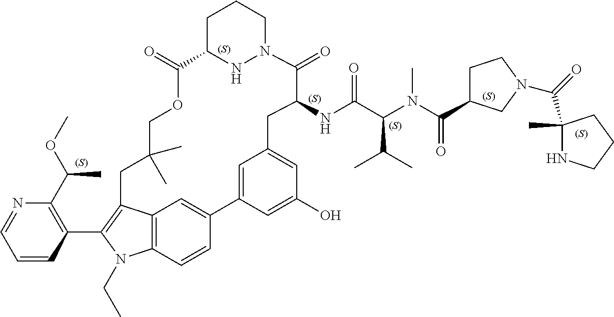 |
| AA381 | 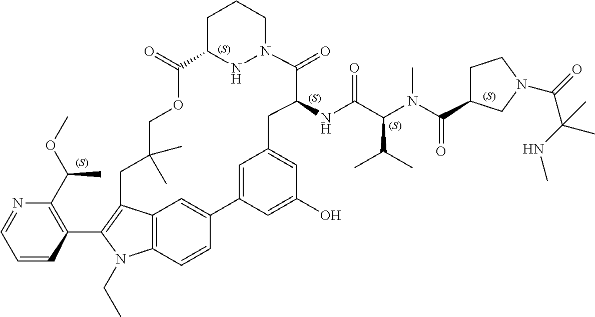 |
| AA382 | 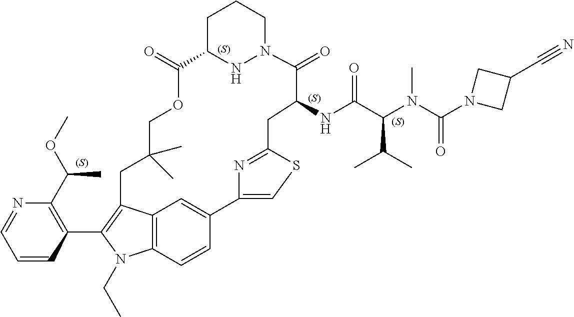 |
| AA383 | 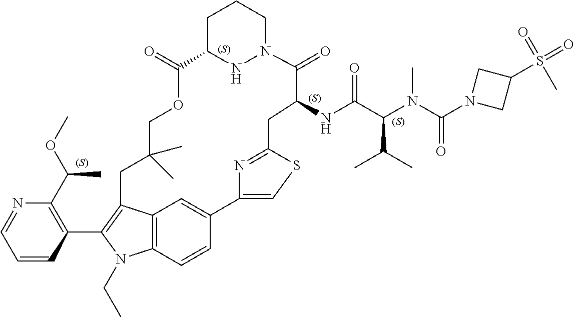 |
| AA384 | 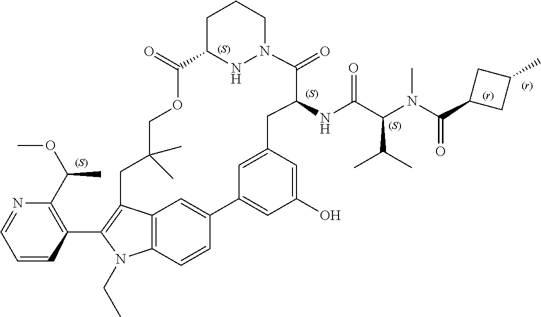 |
| AA385 | 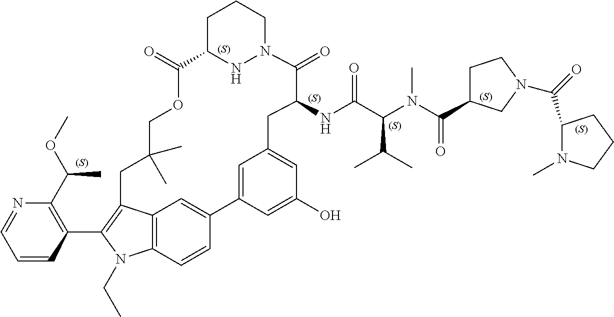 |
| AA386 | 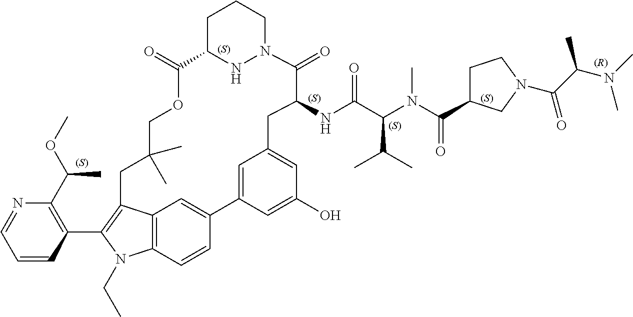 |
| AA387 | 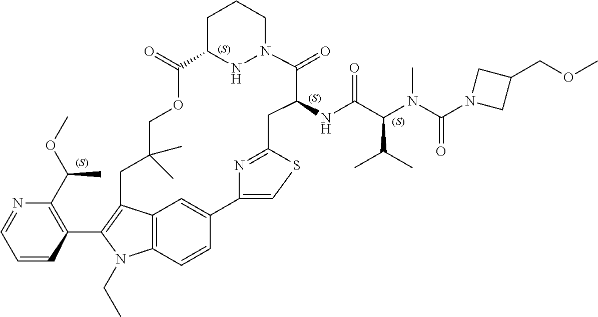 |
| AA388 | 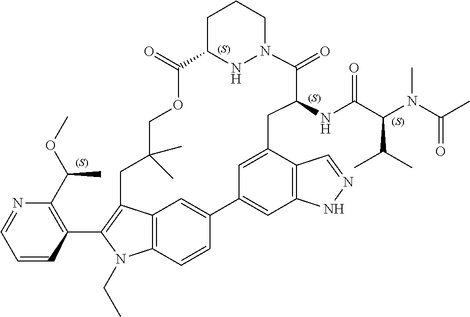 |
| AA389 | 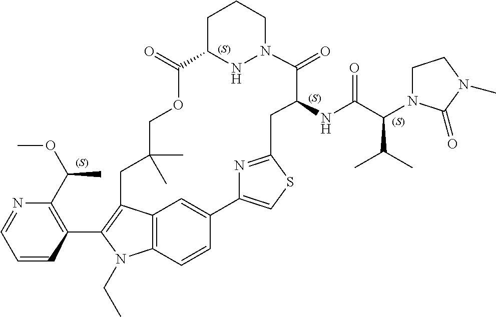 |
| AA391 | 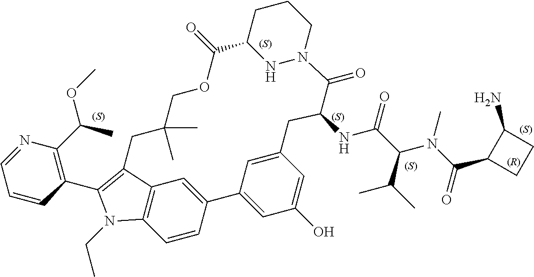 |
| AA392 | 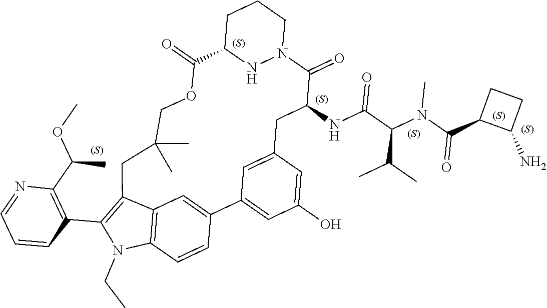 |
| AA393 | 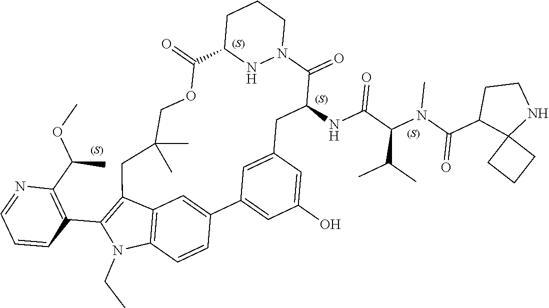 |
| AA394 | 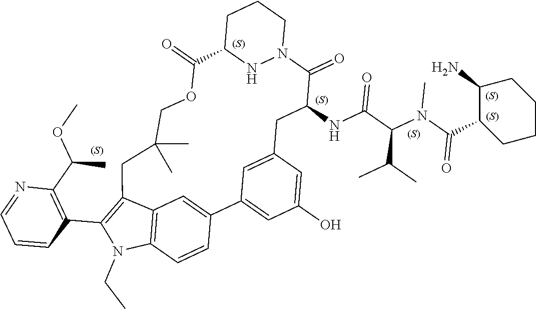 |
| AA395 | 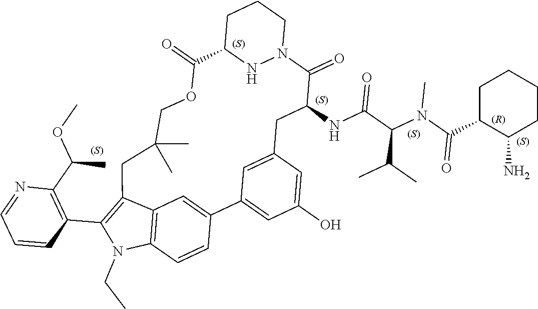 |
| AA396 | 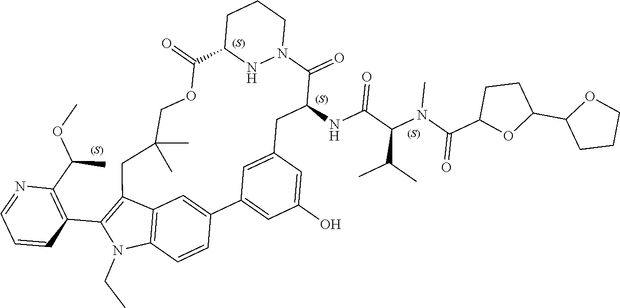 |
| AA397 | 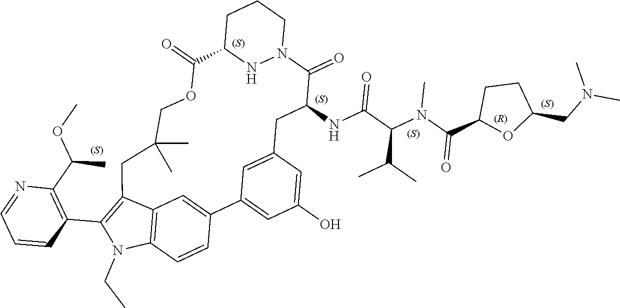 |
| AA398 | 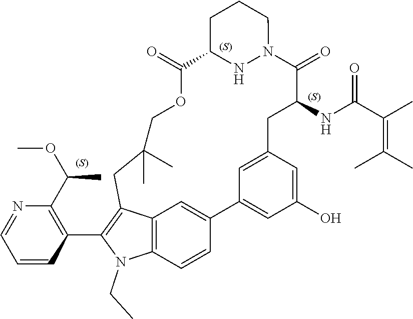 |
| AA399 | 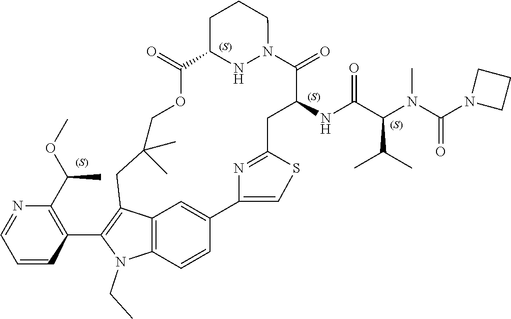 |
| AA400 | 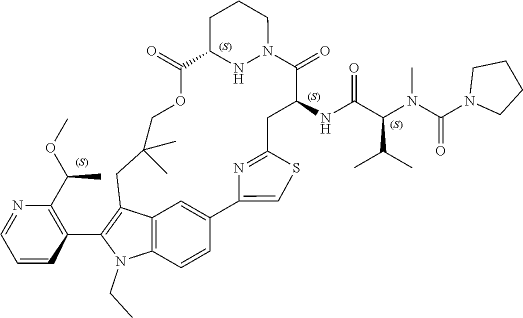 |
| AA401 | 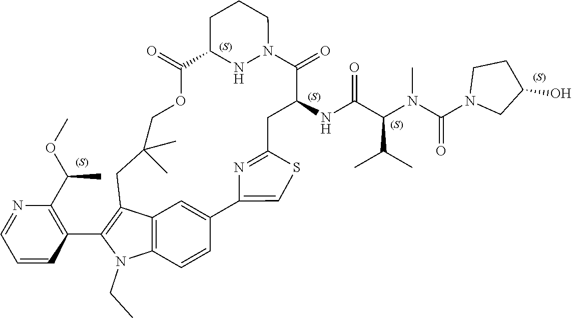 |
| AA402 | 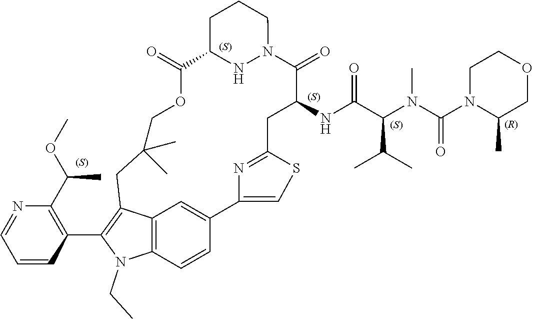 |
| AA403 | 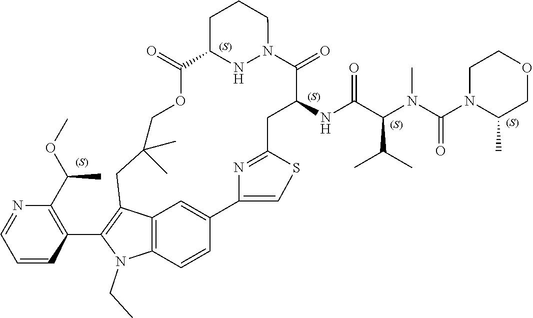 |
| AA404 | 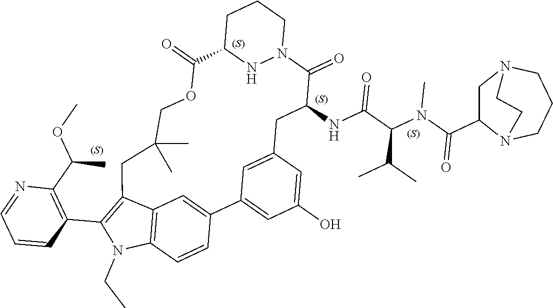 |
| AA405 | 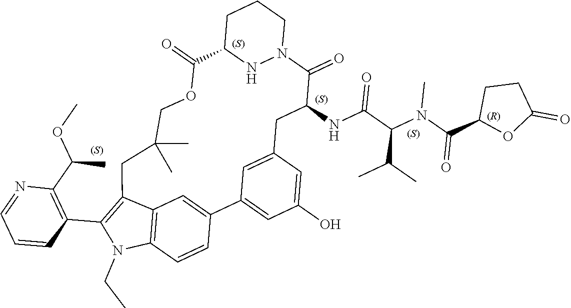 |
| AA406 | 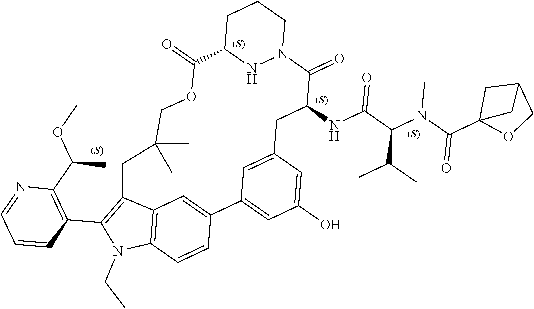 |
| AA407 | 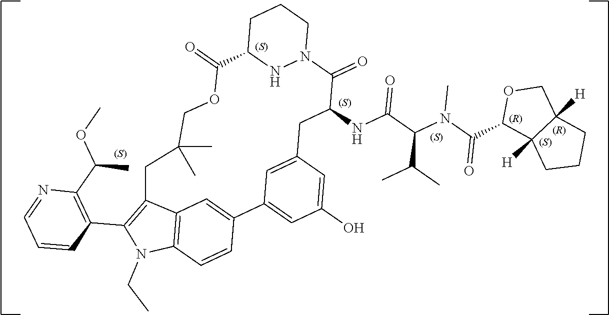 |
| AA408 | 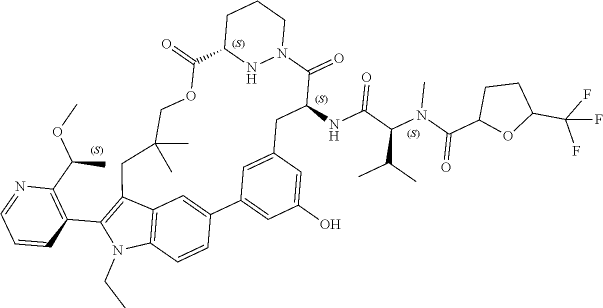 |
| AA409 | 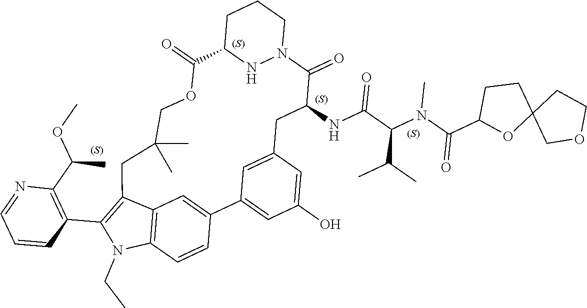 |
| AA410 | 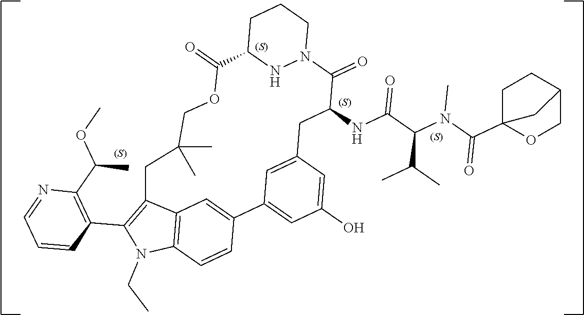 |
| AA411 | 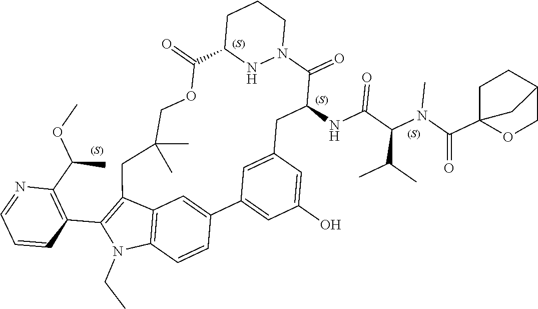 |
| AA412 | 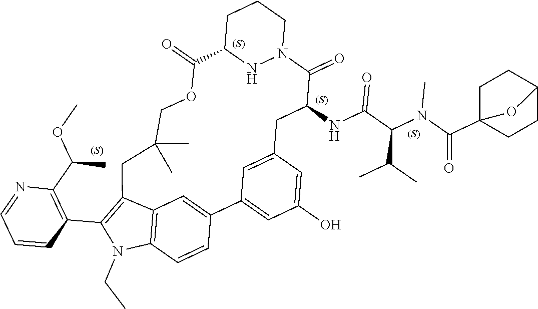 |
| AA413 | 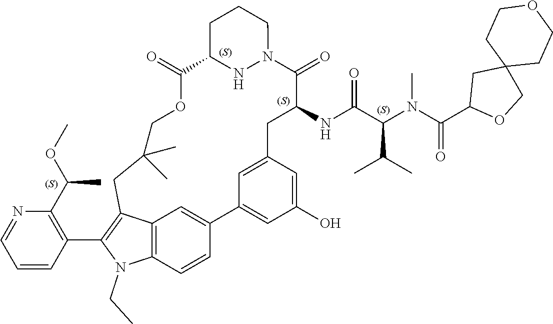 |
| AA414 | 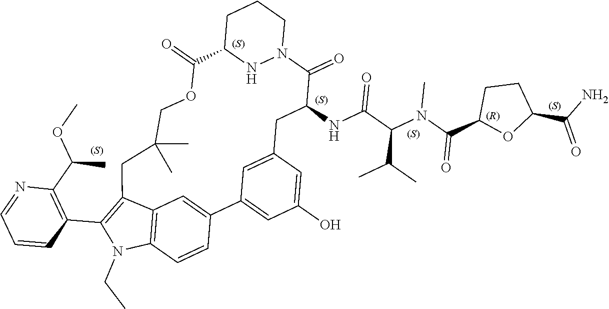 |
| AA415 | 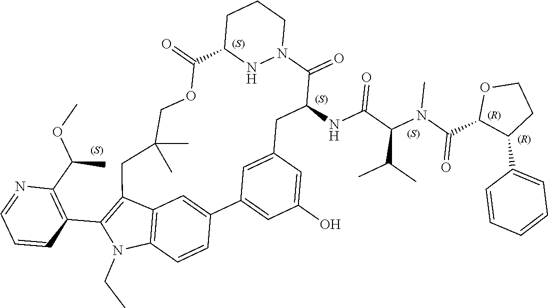 |
| AA416 | 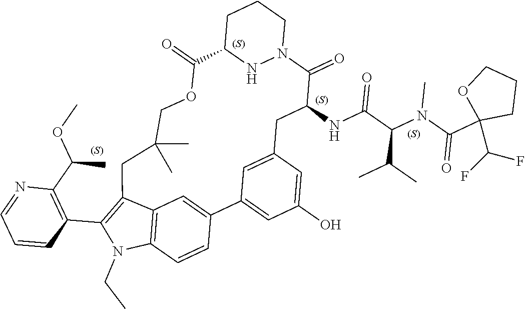 |
| AA417 | 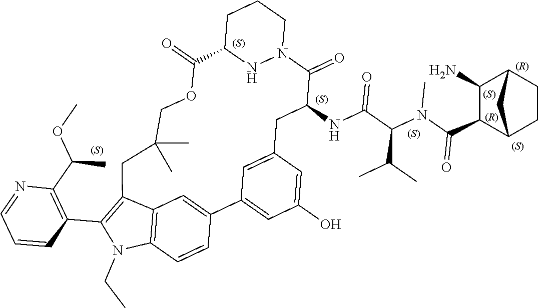 |
| AA418 | 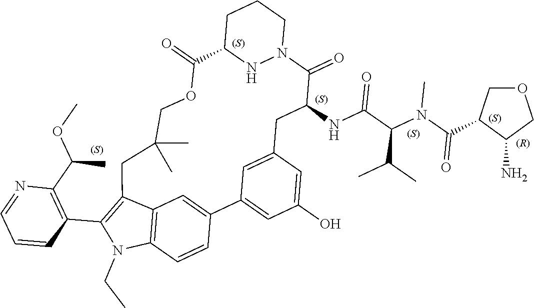 |
| AA419 | 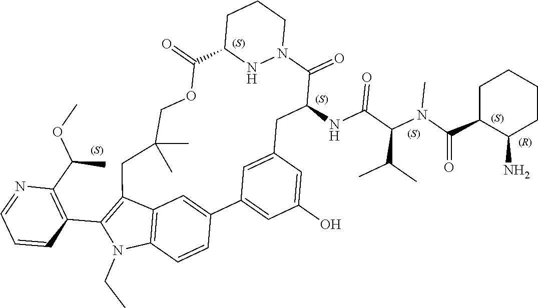 |
| AA420 | 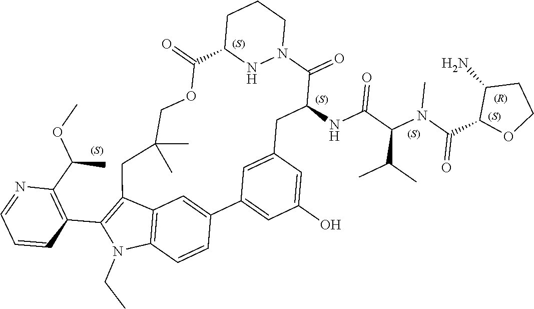 |
| AA421 | 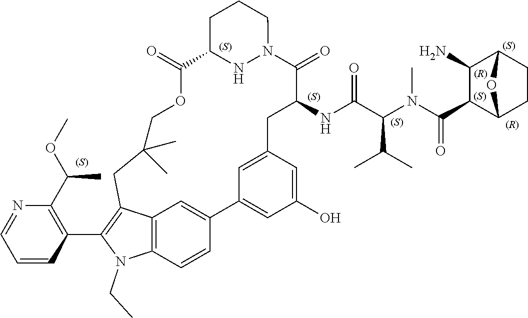 |
| AA422 | 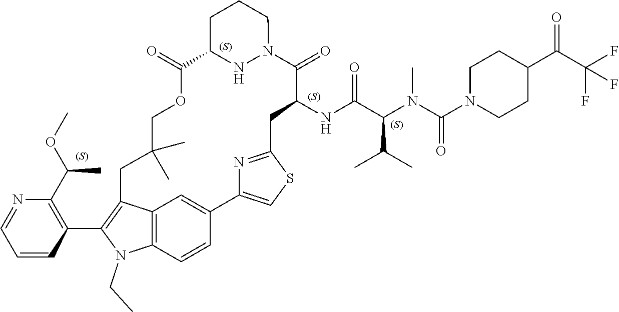 |
| AA423 | 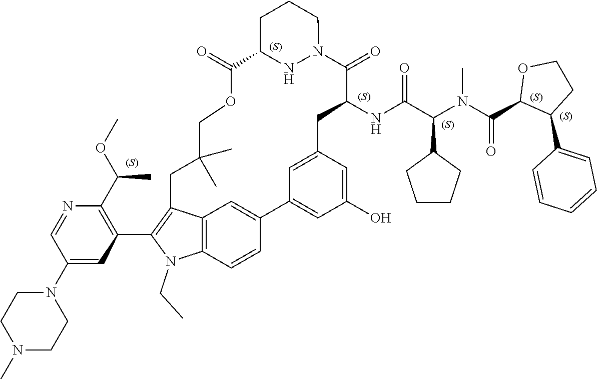 |
| AA424 | 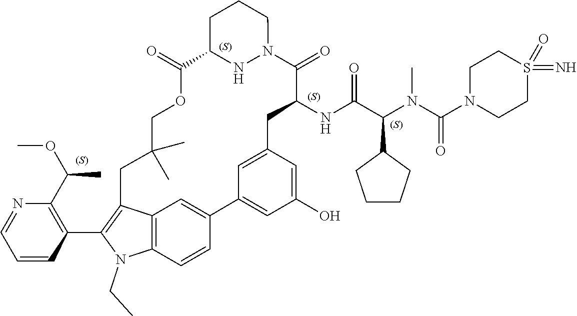 |
| AA425 | 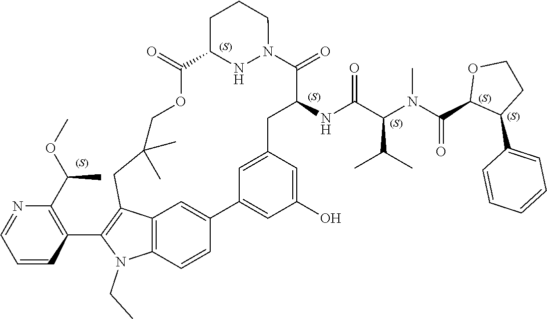 |
| AA426 | 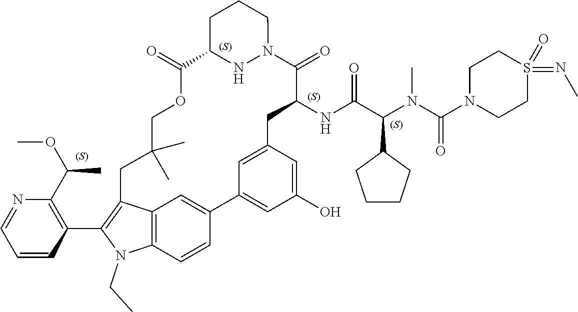 |
| AA427 | 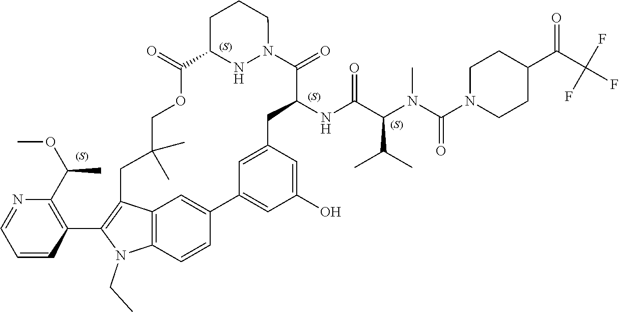 |
| AA428 | 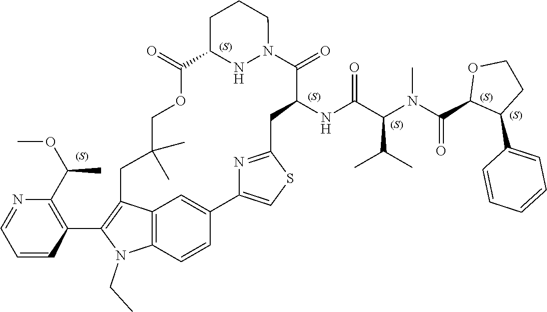 |
| AA429 | 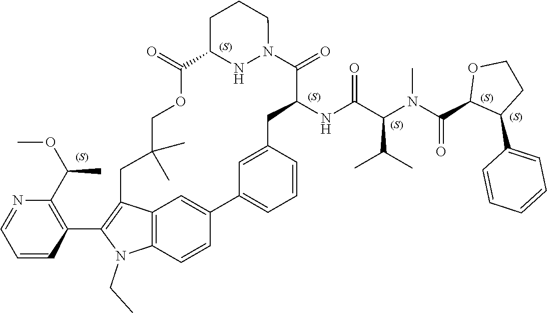 |
| AA430 | 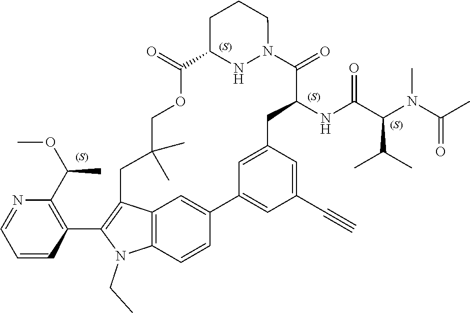 |
| AA431 | 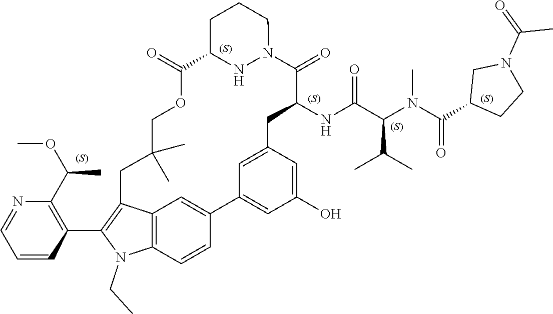 |
| AA432 | 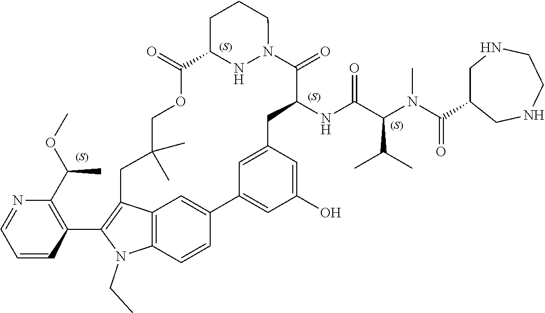 |
| AA433 | 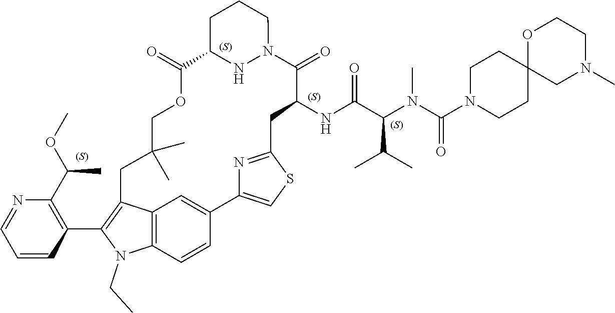 |
| AA434 | 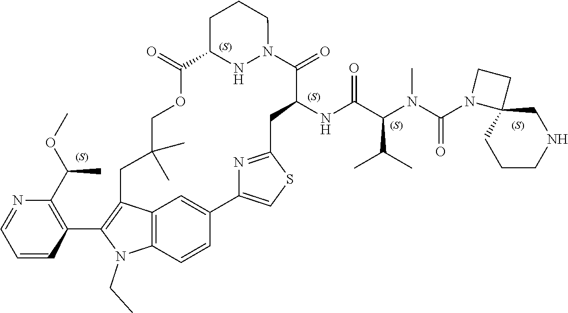 |
| AA435 | 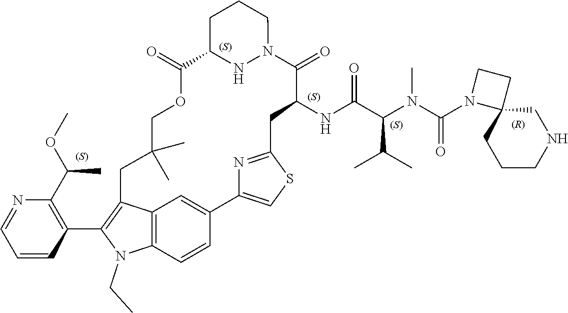 |
| AA436 | 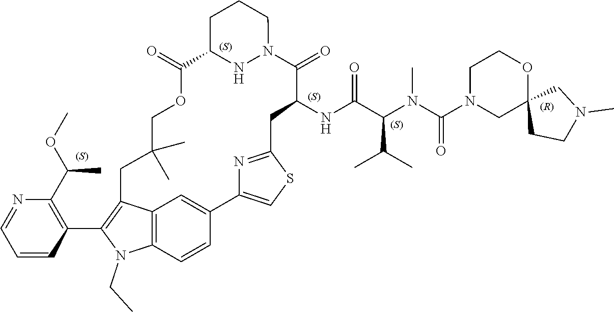 |
| AA437 | 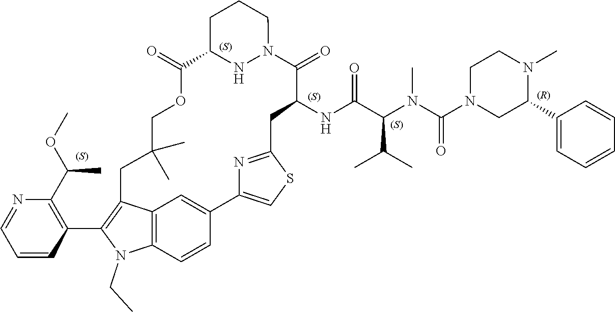 |
| AA438 | 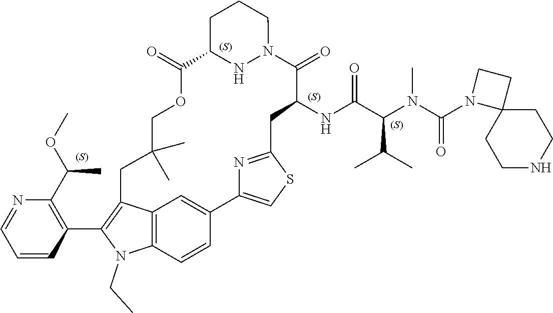 |
| AA439 | 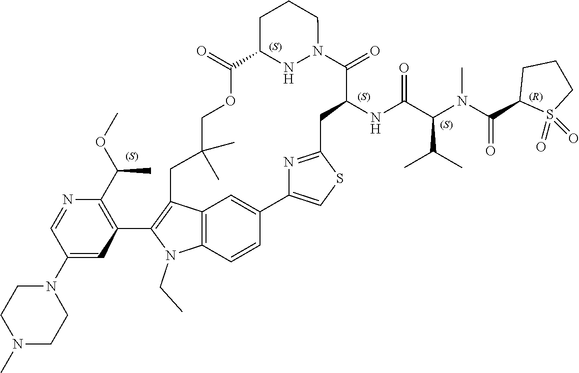 |
| AA440 | 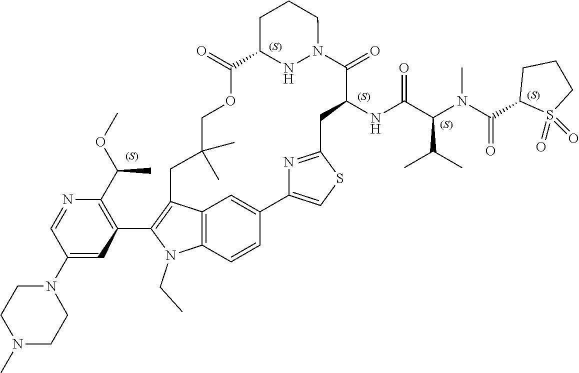 |
| AA441 | 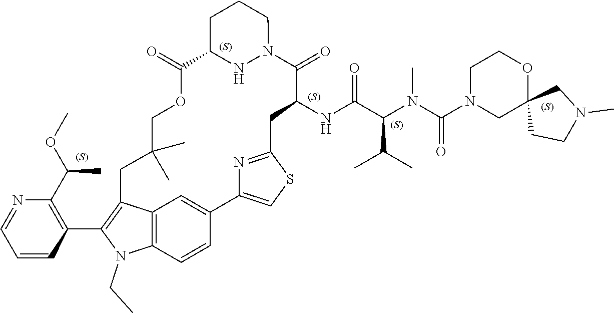 |
| AA442 | 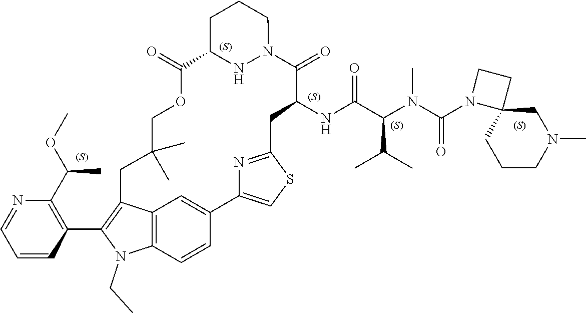 |
| AA443 | 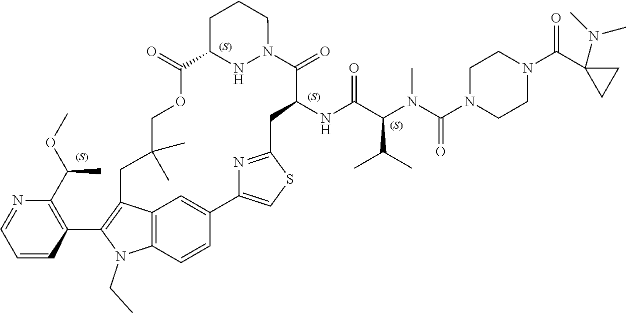 |
| AA444 | 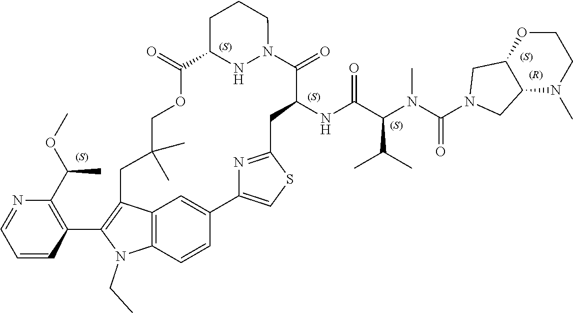 |
| AA445 | 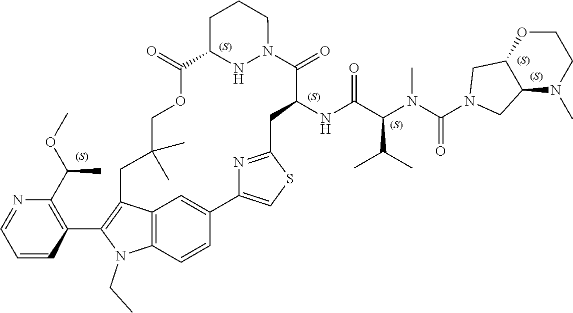 |
| AA446 | 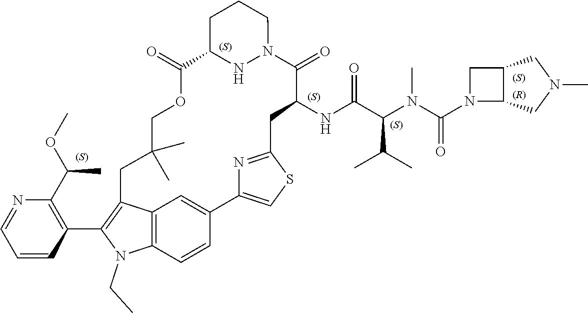 |
| AA447 | 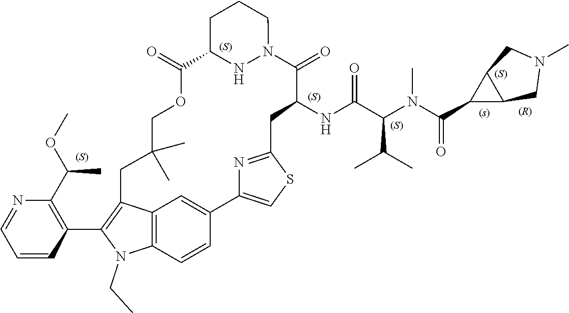 |
| AA448 | 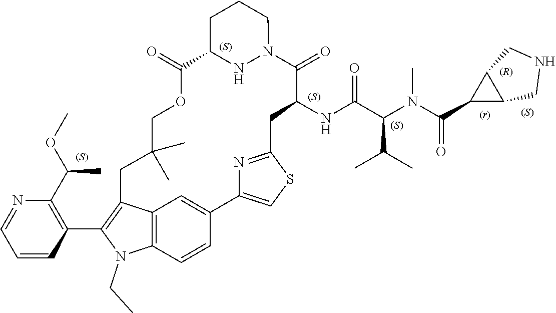 |
| AA449 | 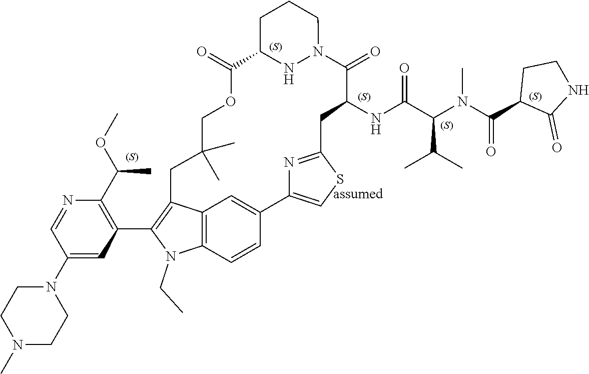 |
| AA450 | 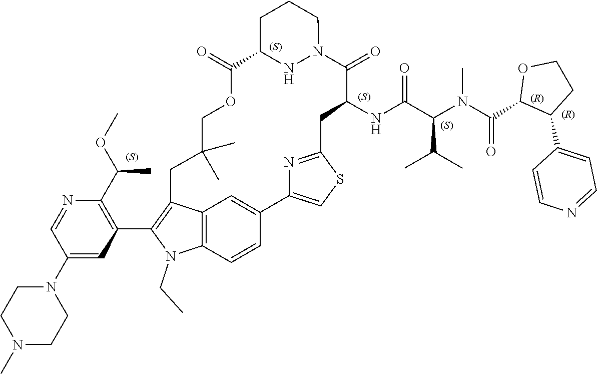 |
| AA451 | 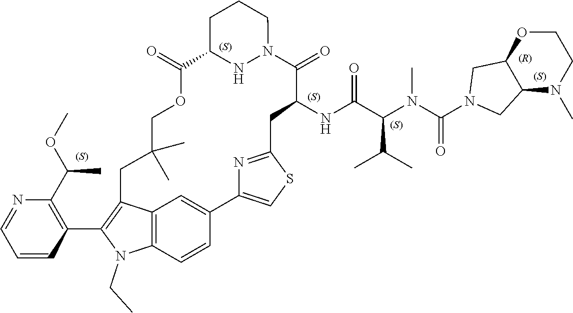 |
| AA452 | 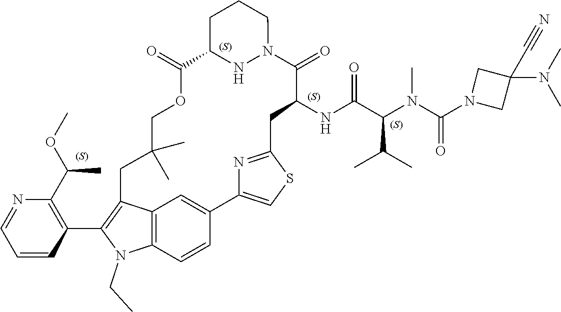 |
| AA453 | 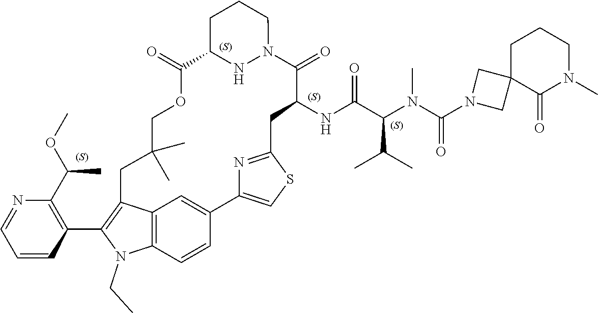 |
| AA454 | 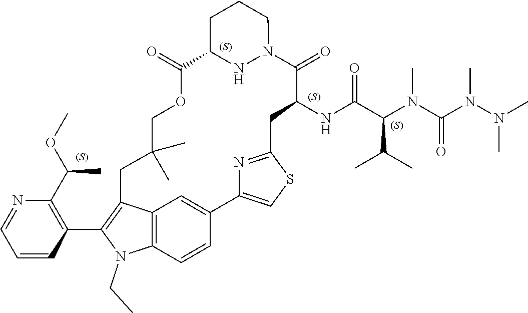 |
| AA455 | 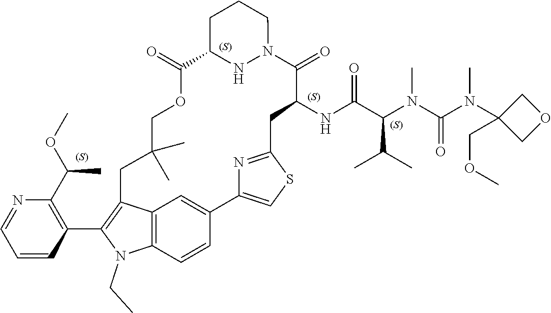 |
| AA456 | 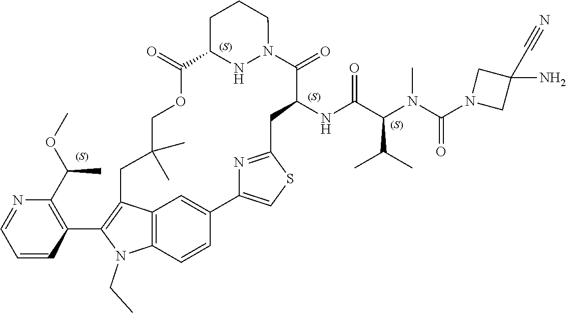 |
| AA457 | 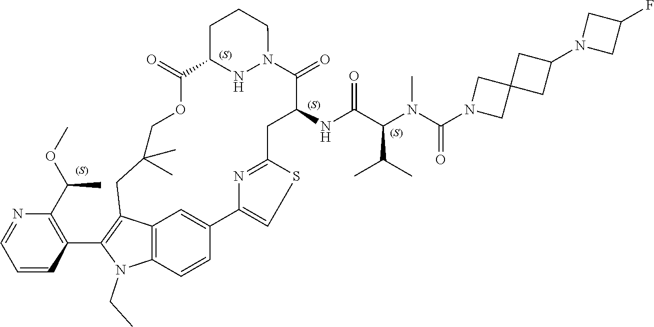 |
| AA458 | 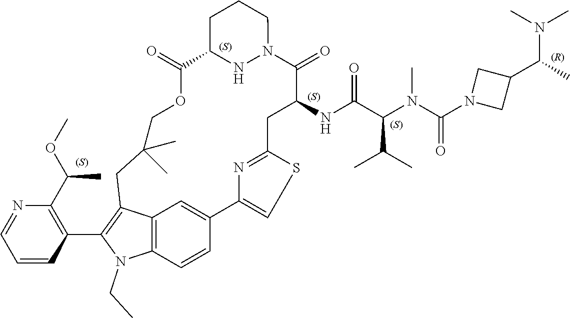 |
| AA459 | 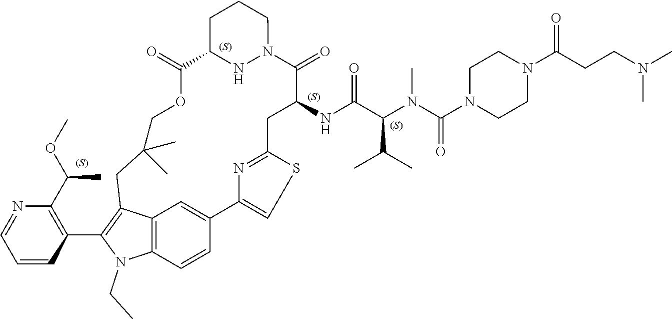 |
| AA460 | 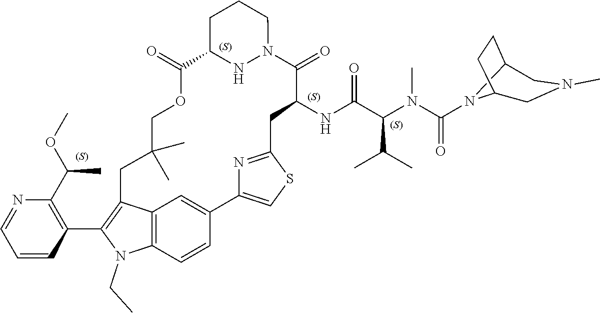 |
| AA461 | 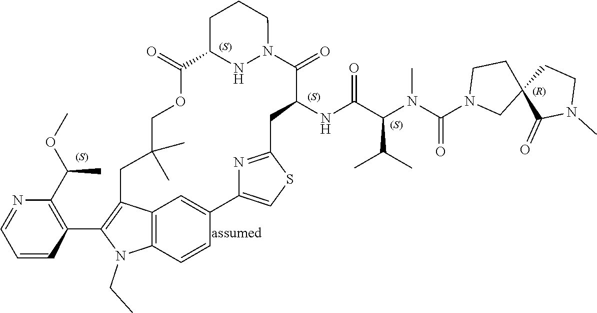 |
| AA462 | 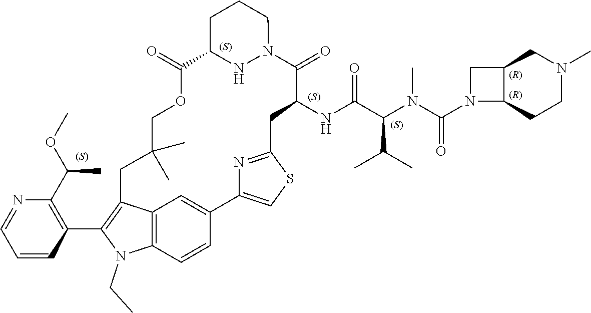 |
| AA463 | 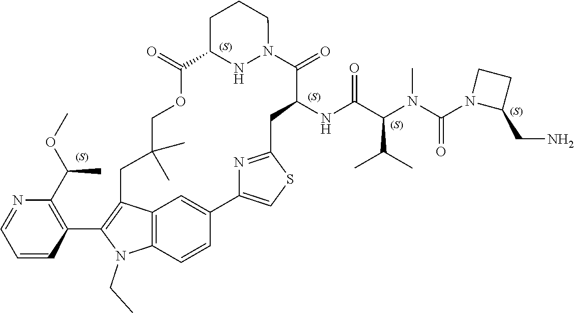 |
| AA464 | 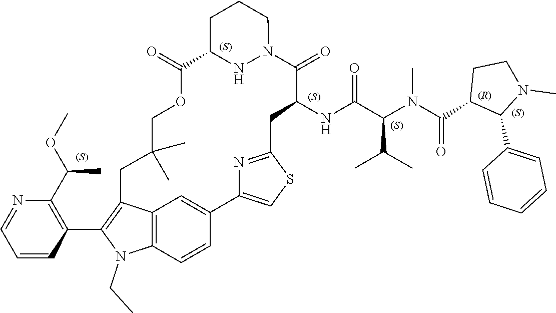 |
| AA465 | 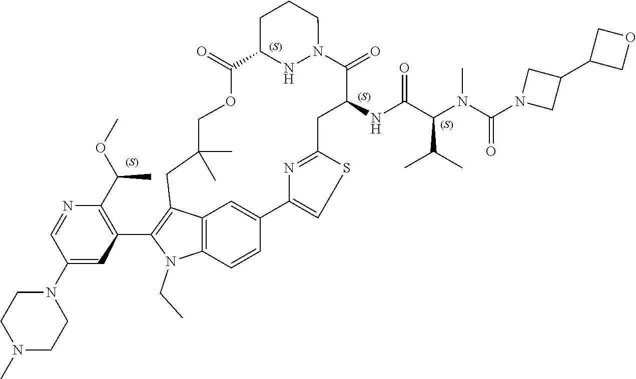 |
| AA466 | 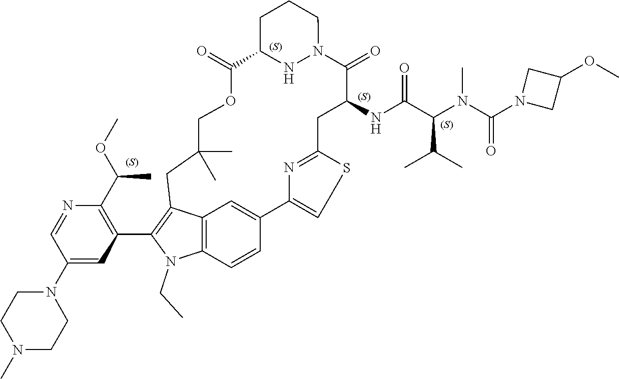 |
| AA467 | 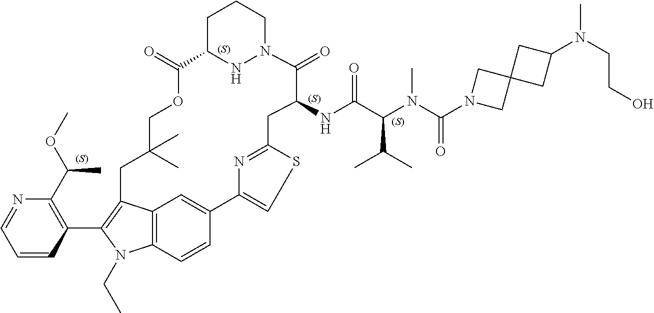 |
| AA468 | 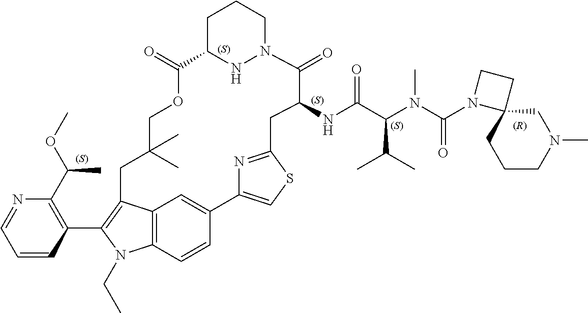 |
| AA469 | 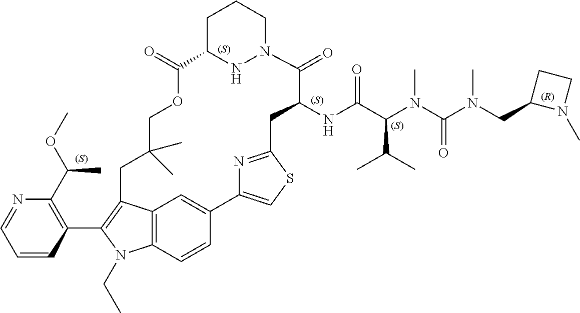 |
| AA470 | 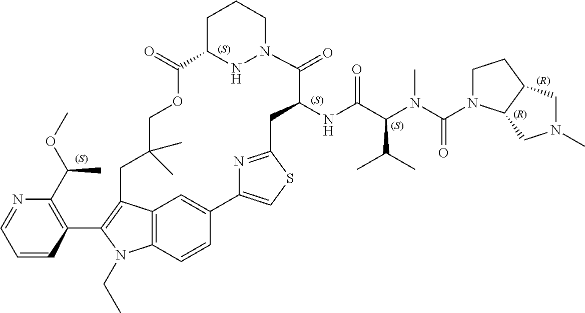 |
| AA471 | 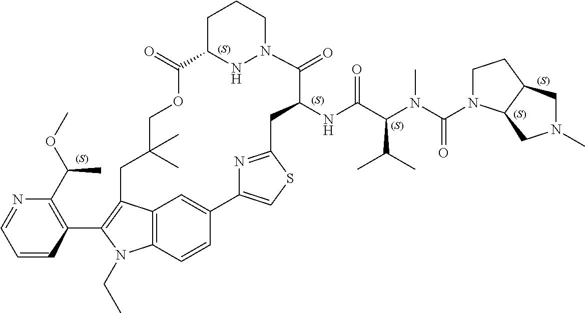 |
| AA472 | 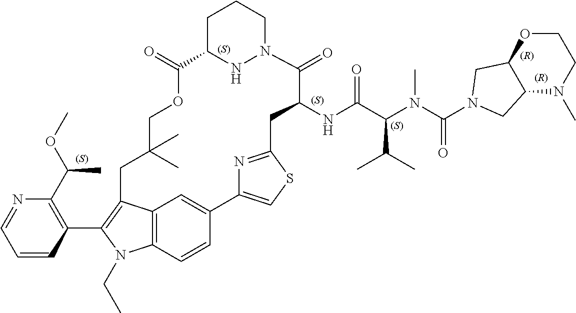 |
| AA473 | 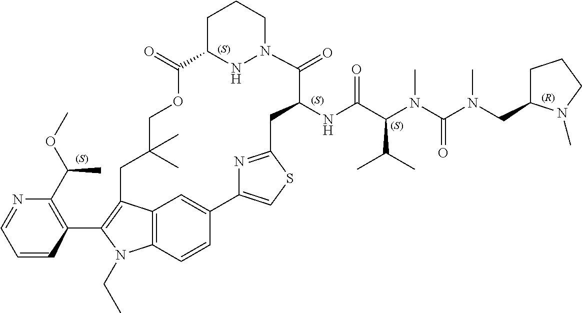 |
| AA474 | 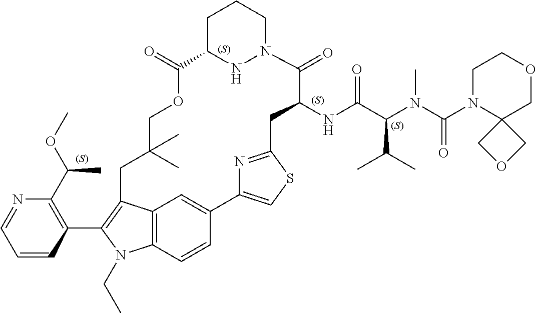 |
| AA475 | 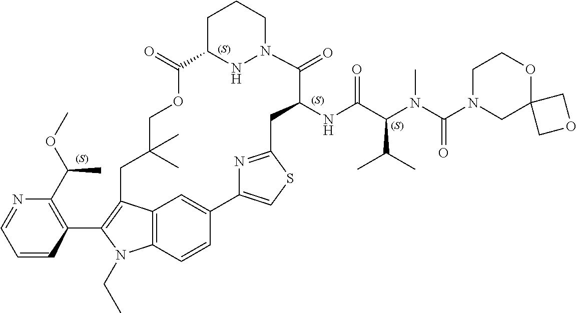 |
| AA476 | 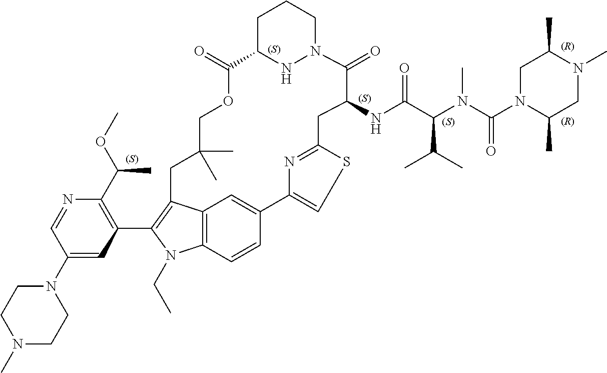 |
| AA477 | 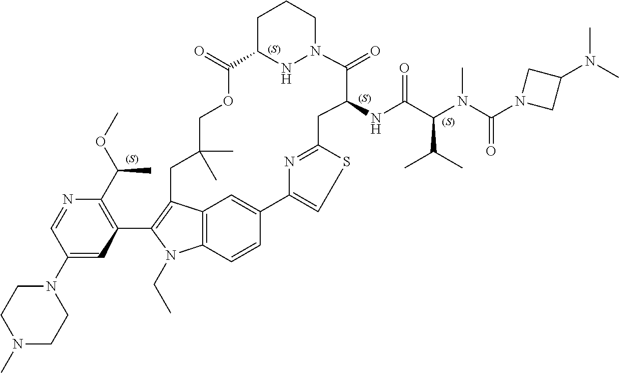 |
| AA478 | 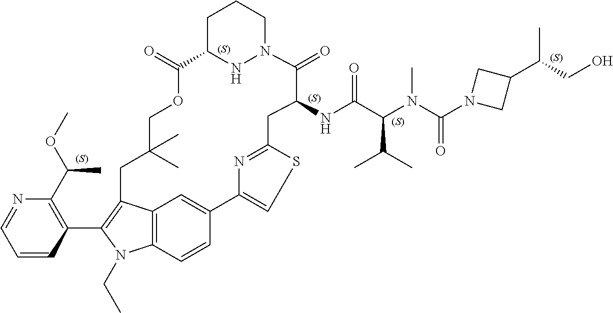 |
| AA479 | 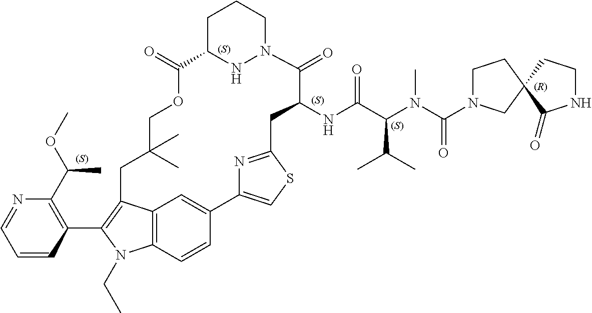 |
| AA480 | 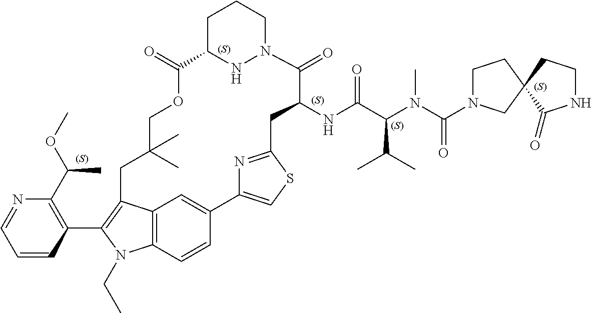 |
| AA481 | 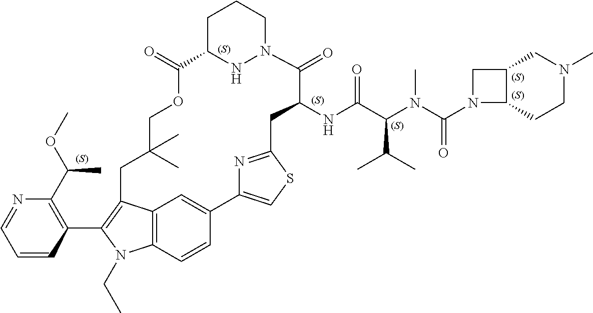 |
| AA482 | 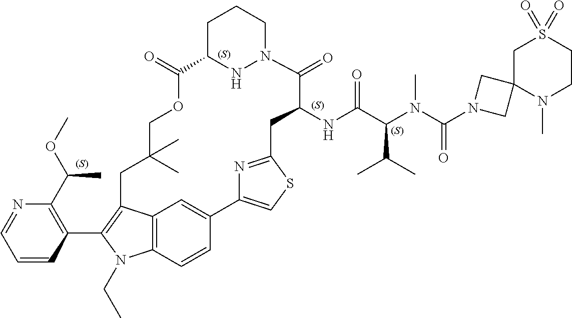 |
| AA483 | 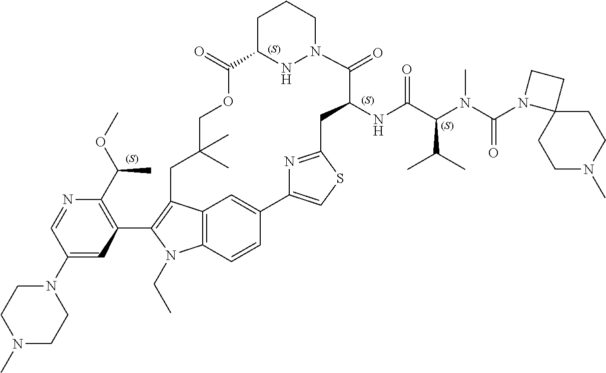 |
| AA484 | 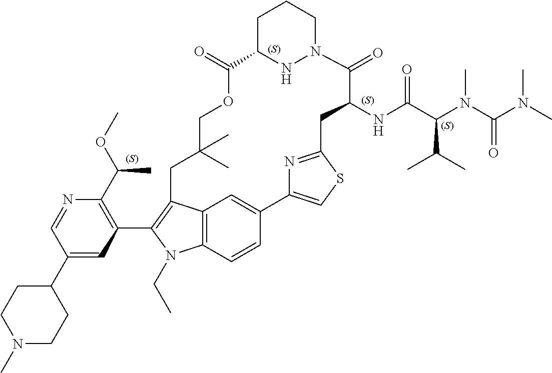 |
| AA485 | 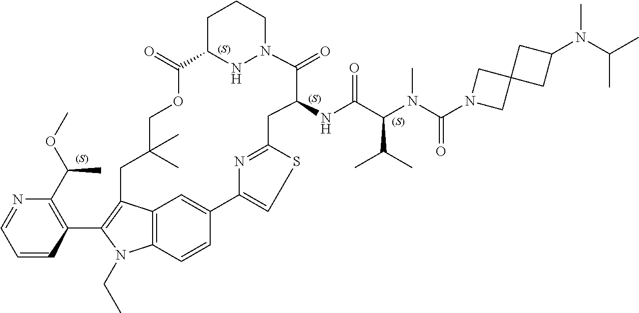 |
| AA486 | 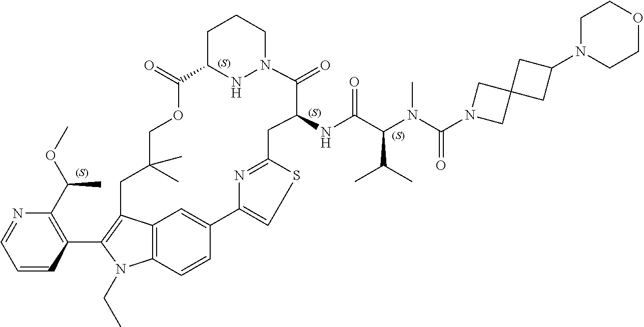 |
| AA487 | 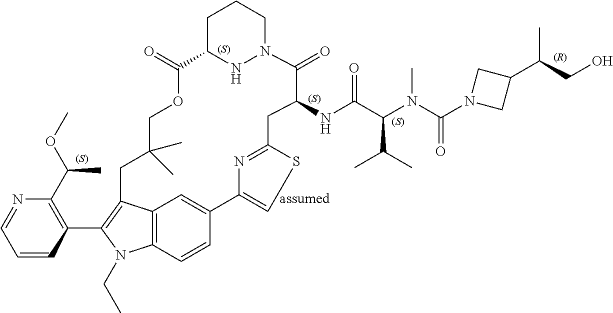 |
| AA488 | 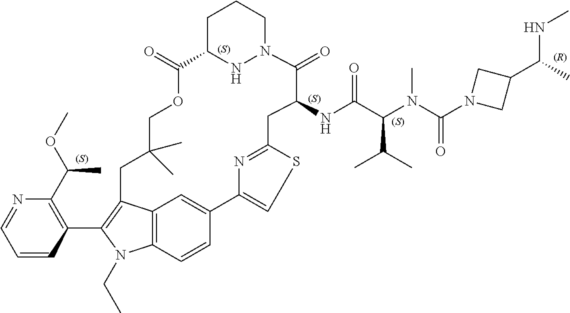 |
| AA489 | 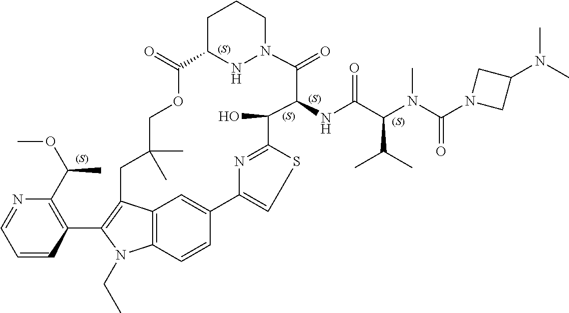 |
| AA490 | 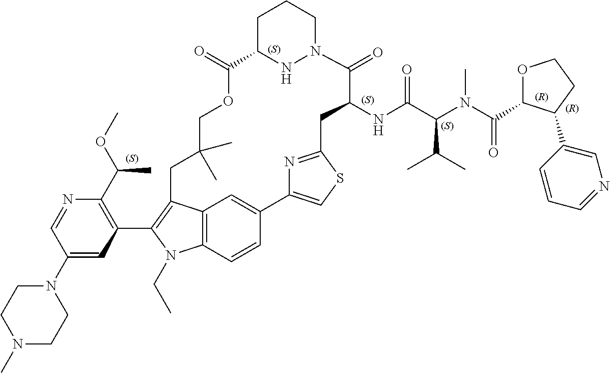 |
| AA491 | 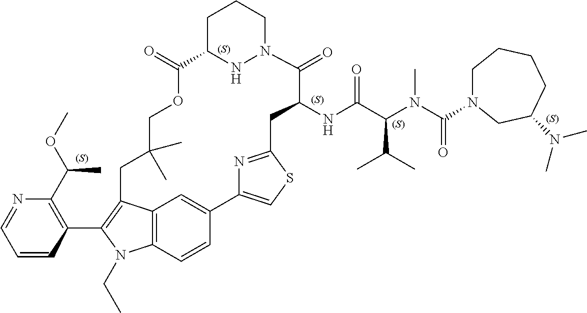 |
| AA492 | 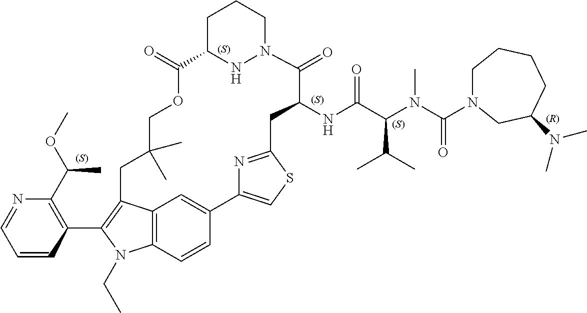 |
| AA493 | 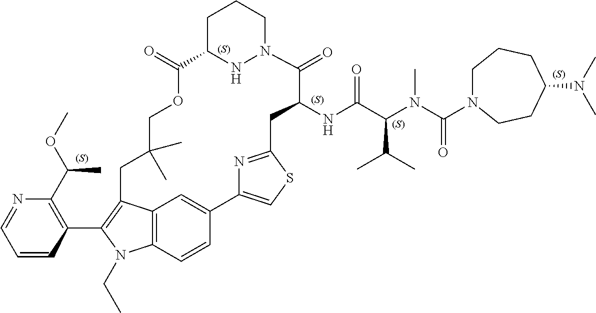 |
| AA494 | 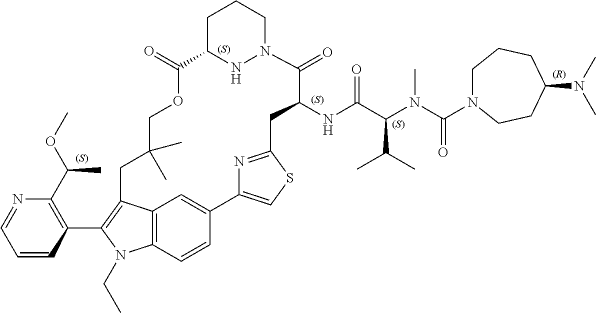 |
| AA495 | 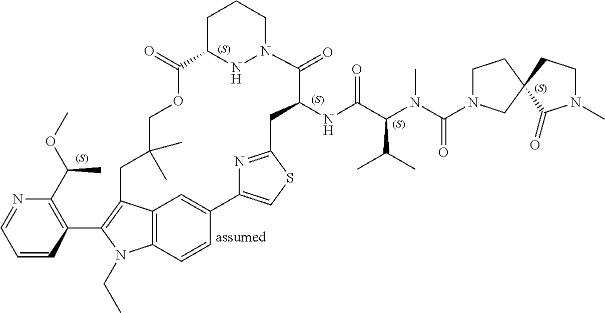 |
| AA496 | 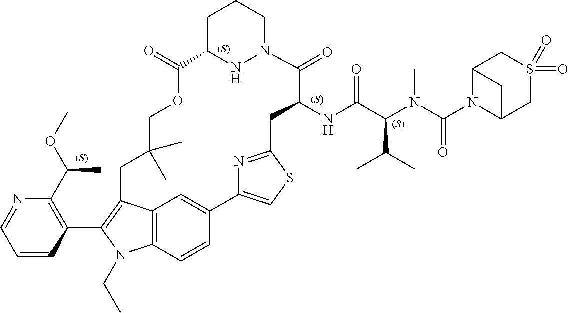 |
| AA497 | 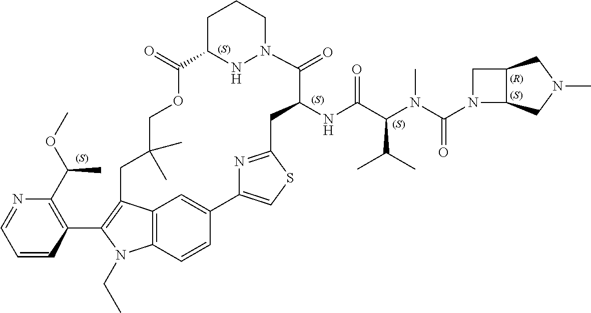 |
| AA498 | 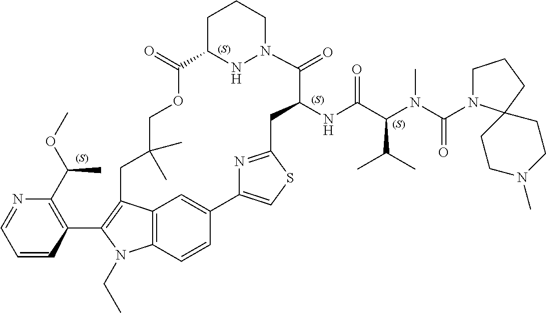 |
| AA499 | 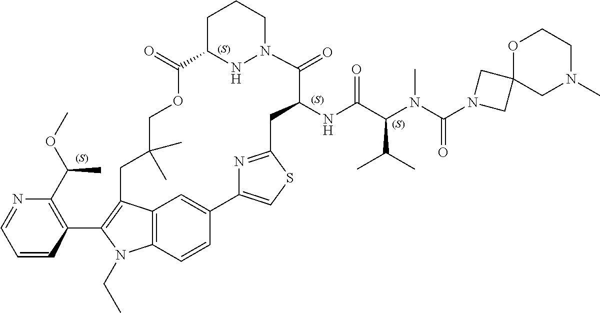 |
| AA500 | 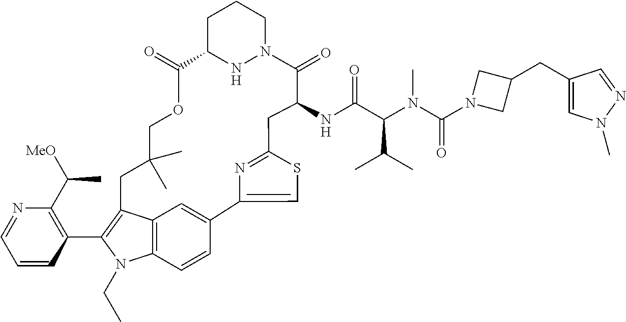 |
| AA501 | 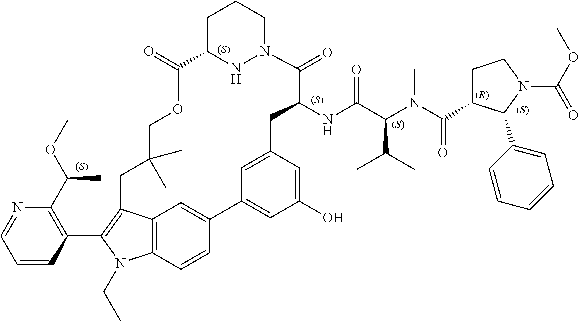 |
| AA502 | 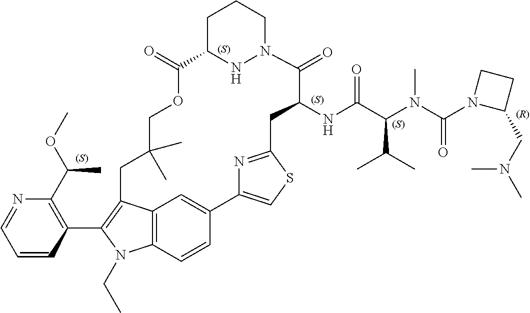 |
| AA503 | 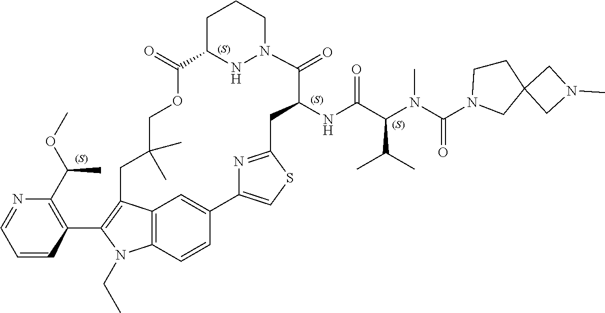 |
| AA504 | 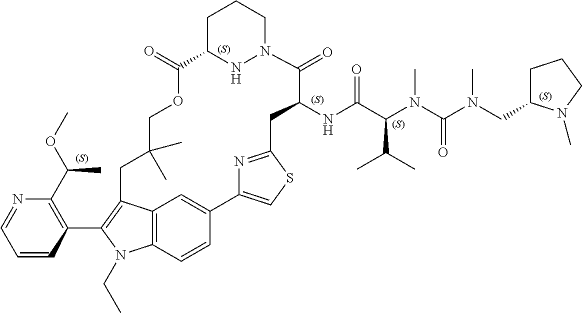 |
| AA505 | 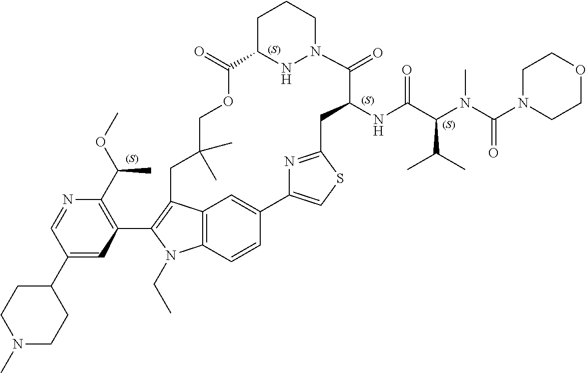 |
| AA506 | 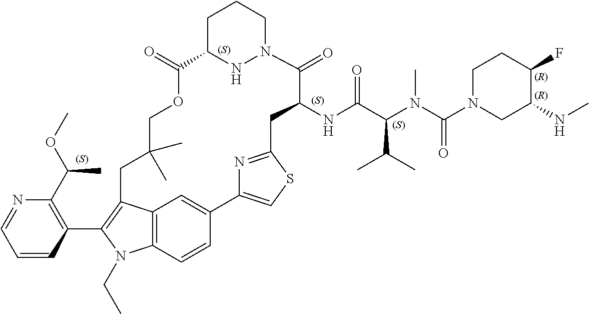 |
| AA507 | 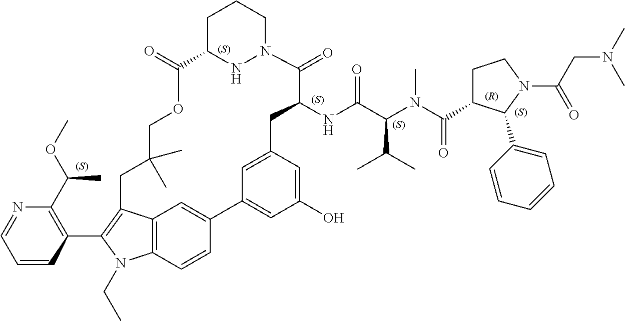 |
| AA508 | 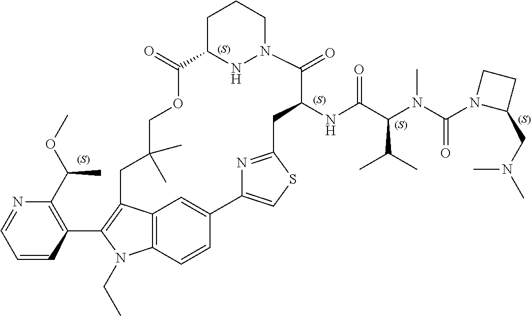 |
| AA509 | 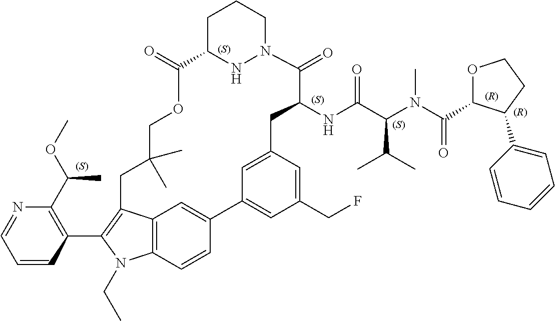 |
| AA510 | 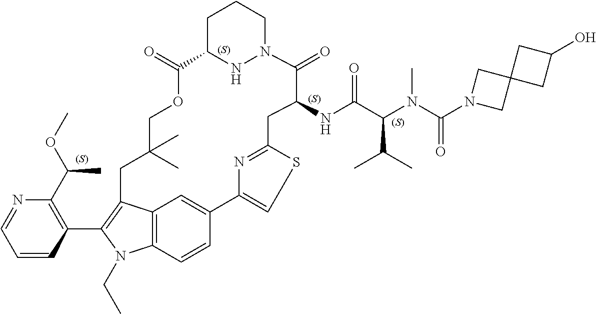 |
| AA511 | 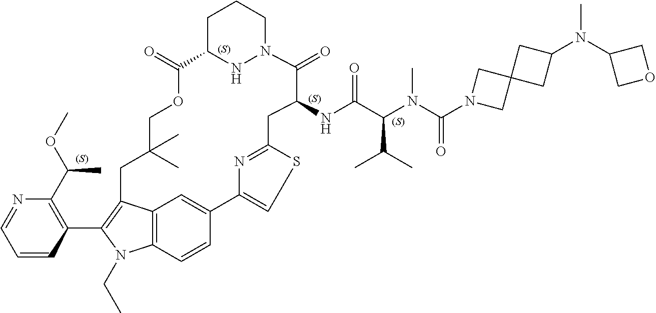 |
| AA512 | 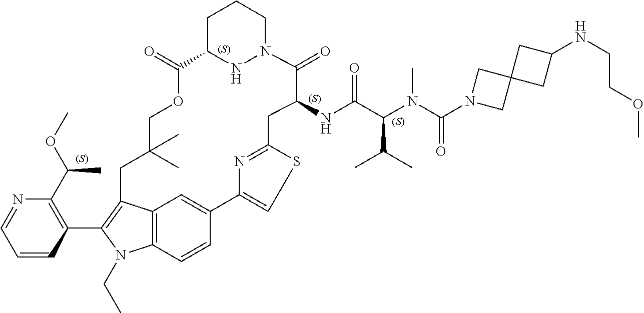 |
| AA513 | 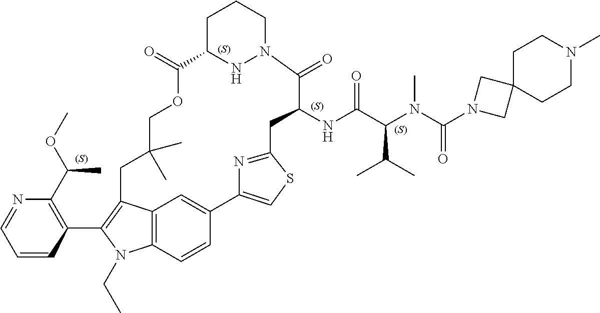 |
| AA514 | 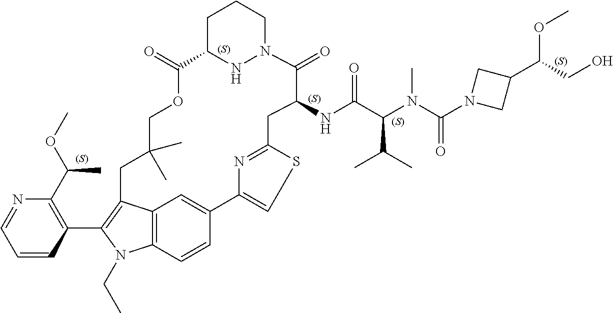 |
| AA515 | 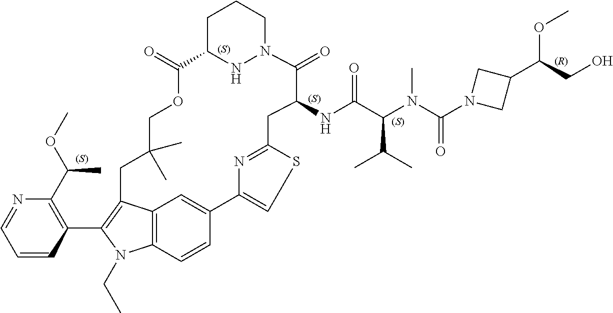 |
| AA516 | 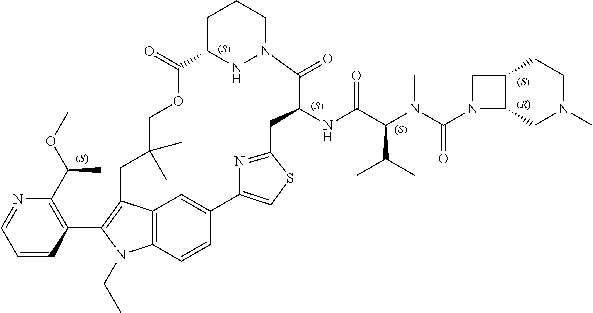 |
| AA517 | 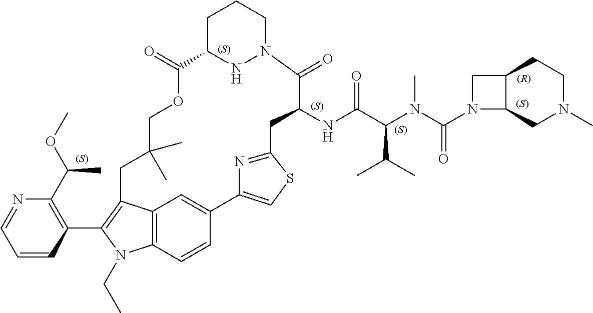 |
| AA518 | 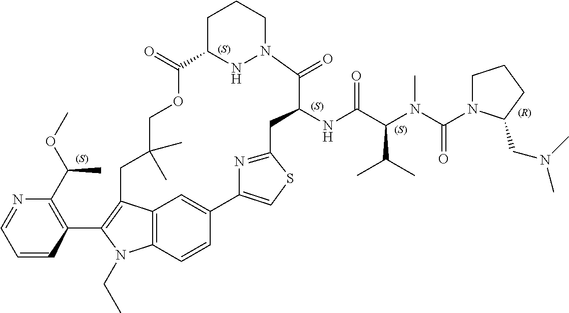 |
| AA519 | 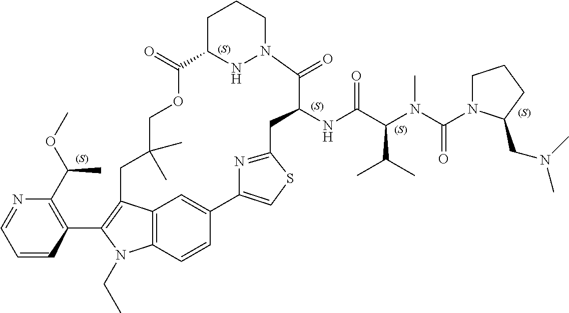 |
| AA520 | 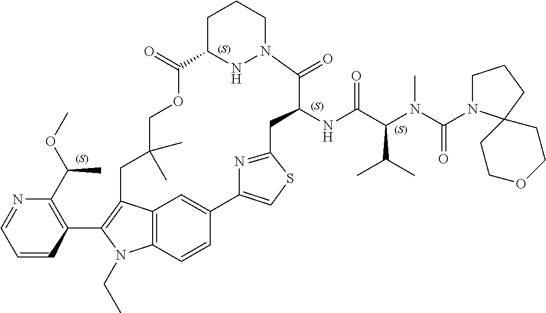 |
| AA521 | 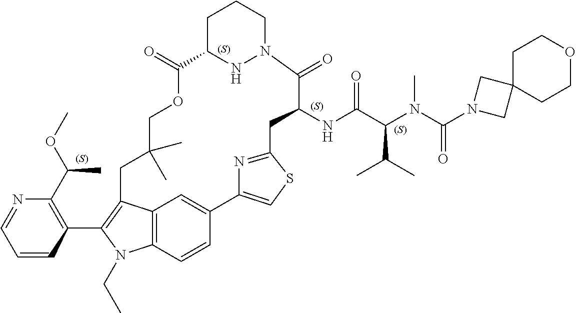 |
| AA522 | 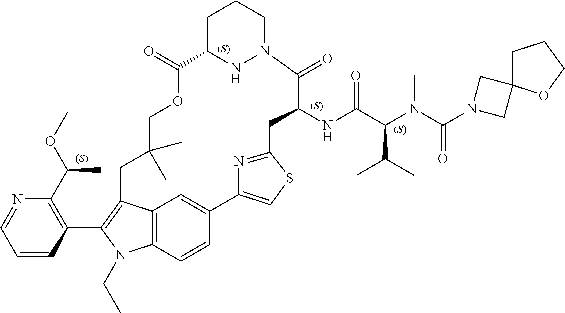 |
| AA523 | 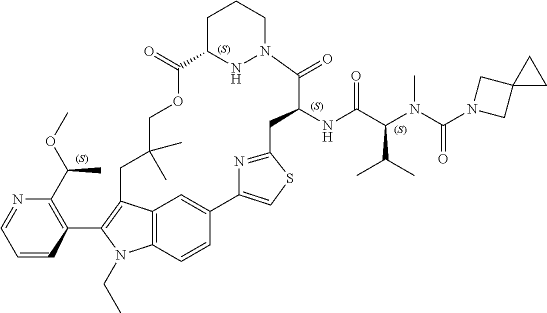 |
| AA524 | 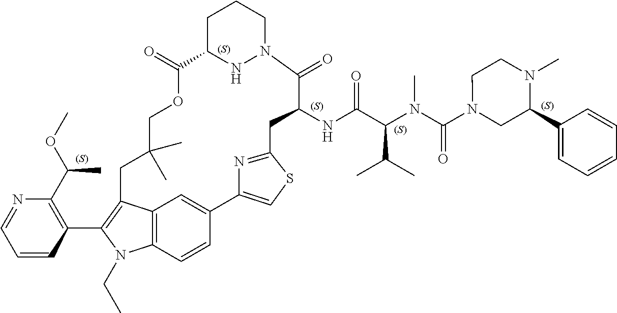 |
| AA525 | 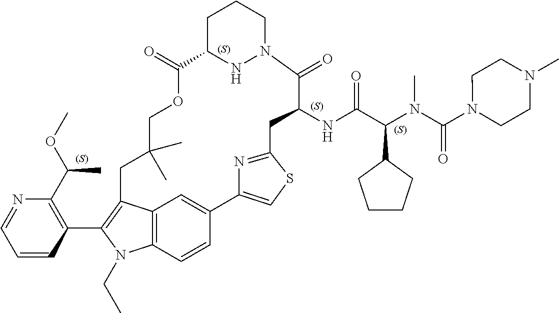 |
| AA526 | 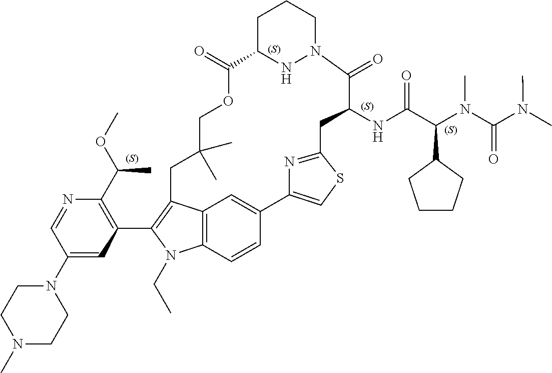 |
| AA527 | 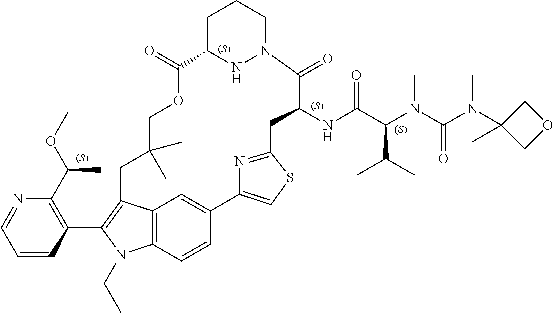 |
| AA528 | 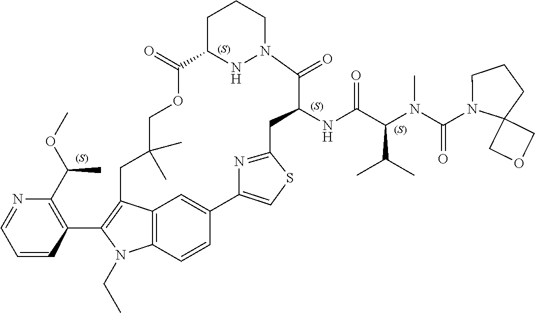 |
| AA529 | 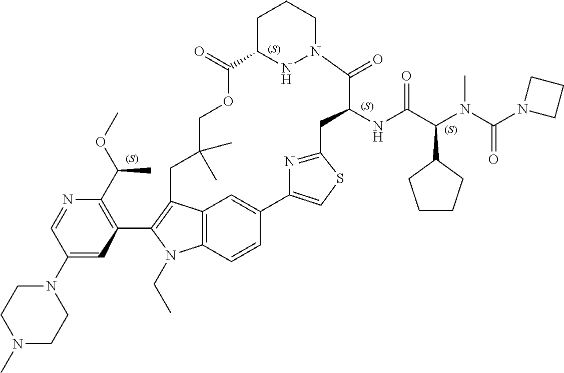 |
| AA530 | 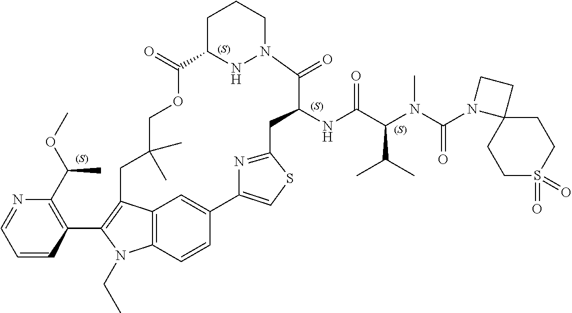 |
| AA531 | 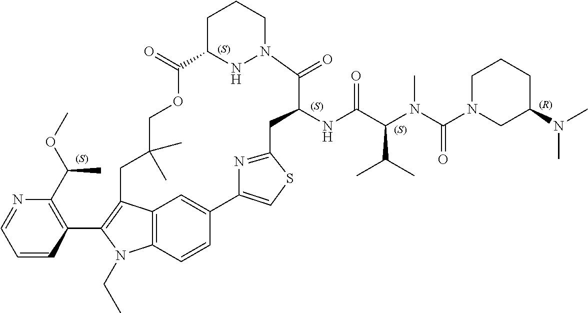 |
| AA532 | 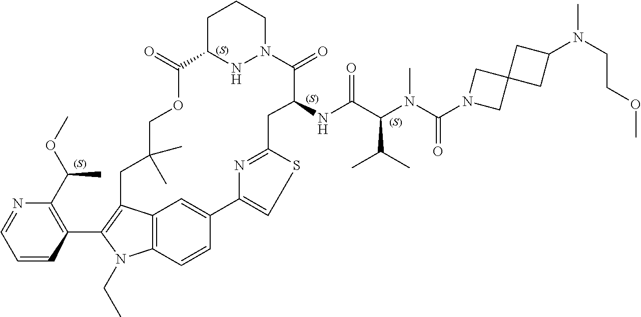 |
| AA533 | 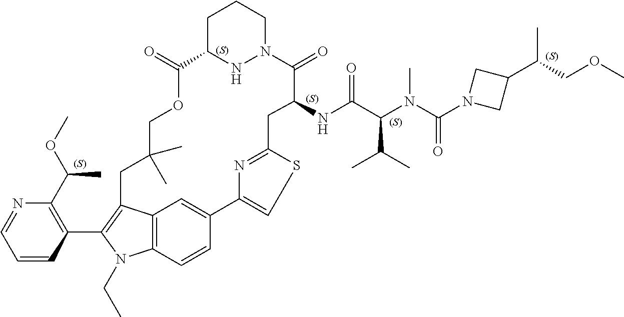 |
| AA534 | 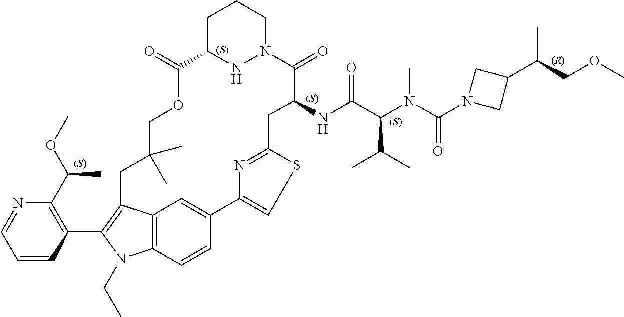 |
| AA535 | 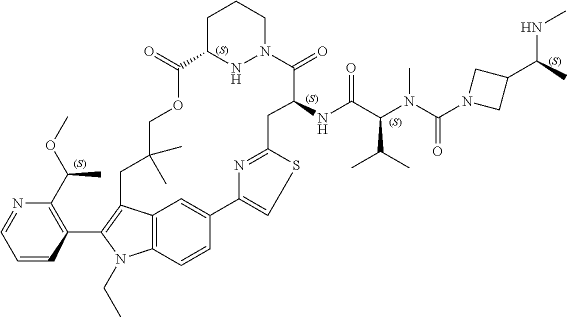 |
| AA536 | 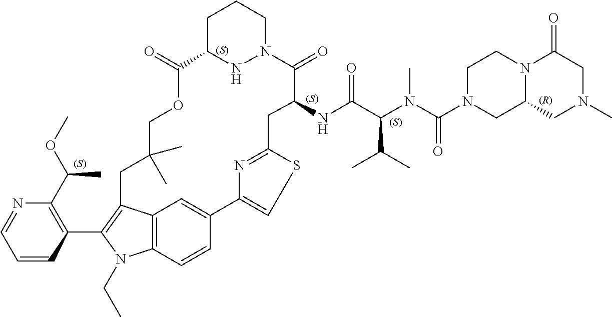 |
| AA537 | 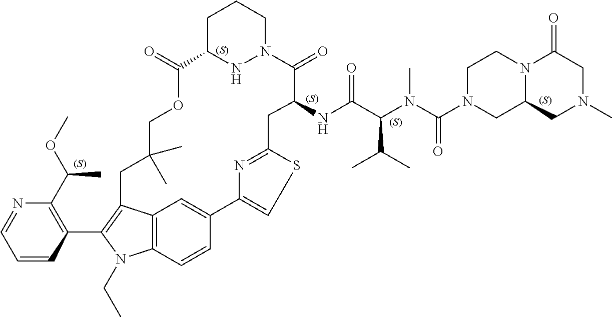 |
| AA538 | 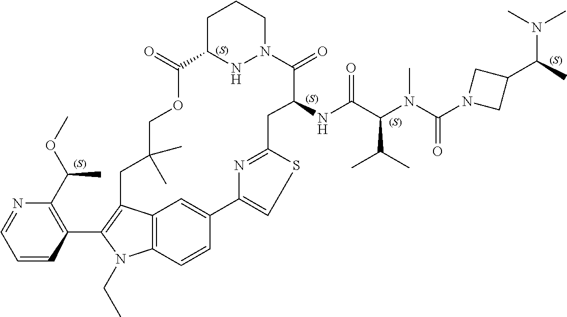 |
| AA539 | 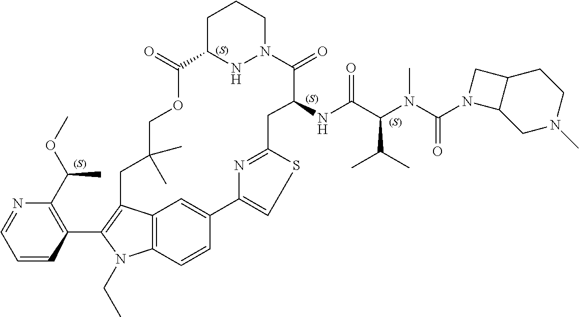 |
| AA540 | 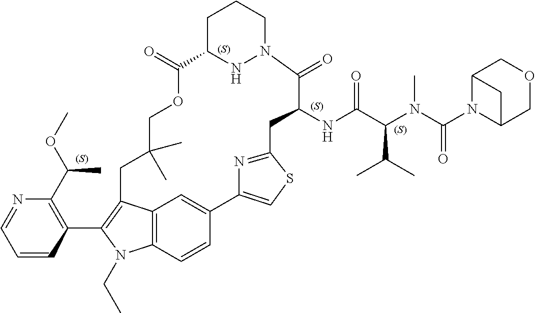 |
| AA541 | 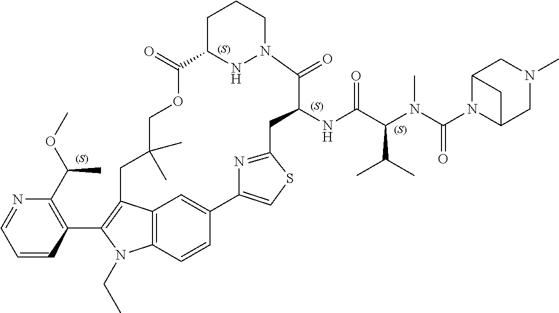 |
| AA542 | 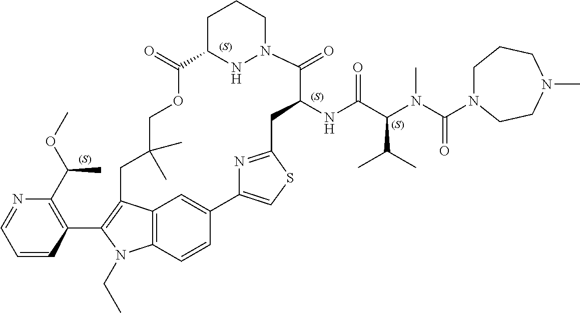 |
| AA543 | 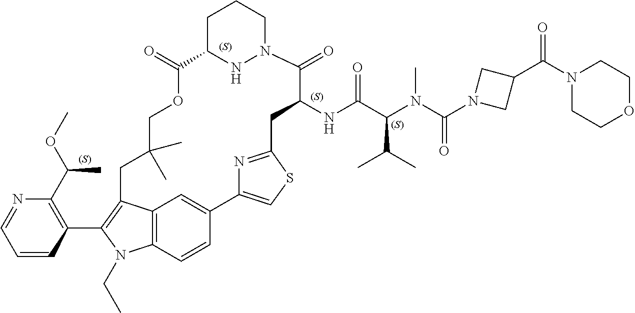 |
| AA544 | 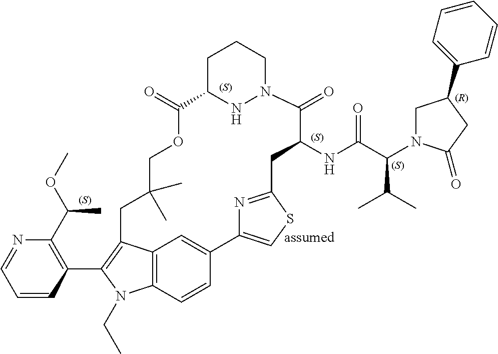 |
| AA545 | 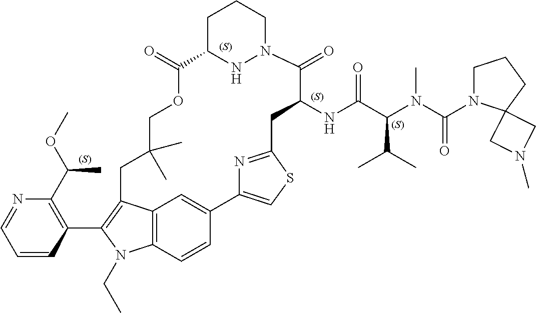 |
| AA546 | 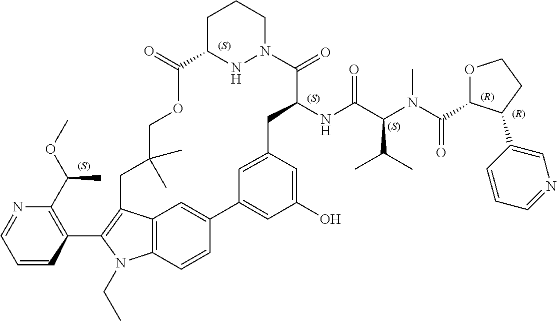 |
| AA547 | 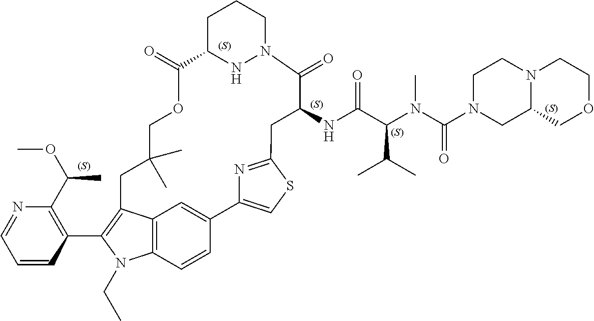 |
| AA548 | 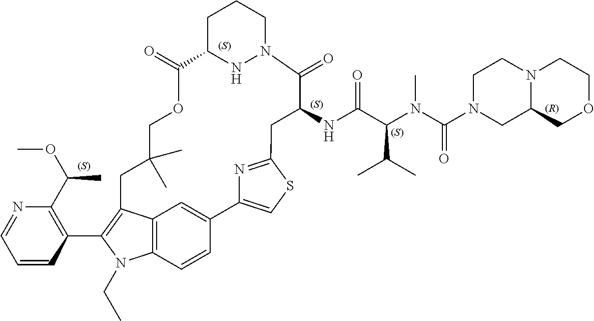 |
| AA549 | 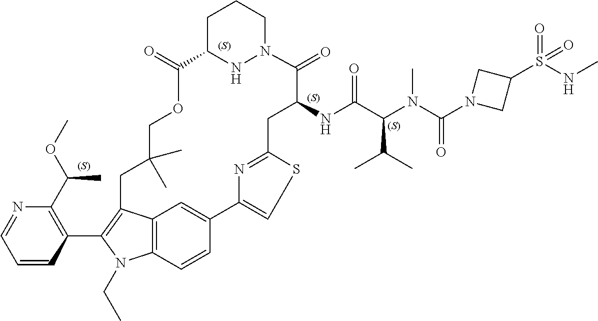 |
| AA550 | 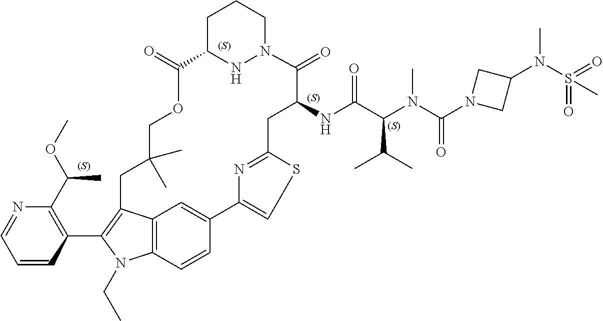 |
| AA551 | 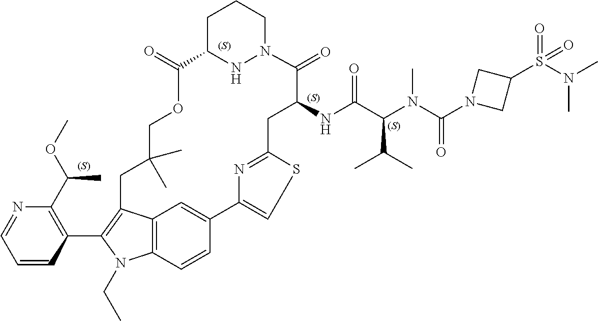 |
| AA552 | 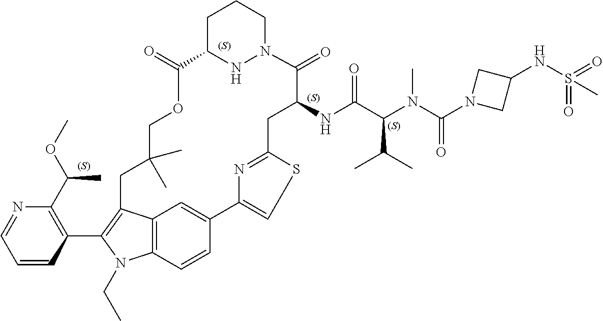 |
| AA553 | 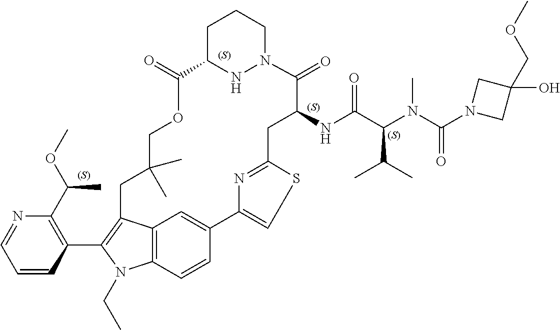 |
| AA554 | 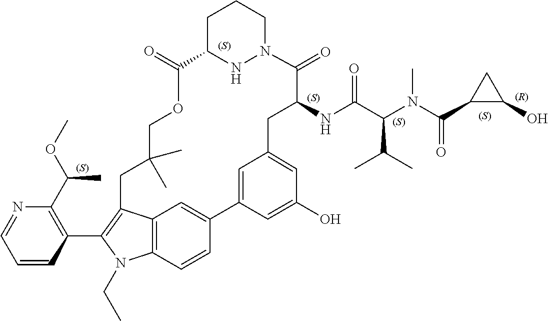 |
| AA555 | 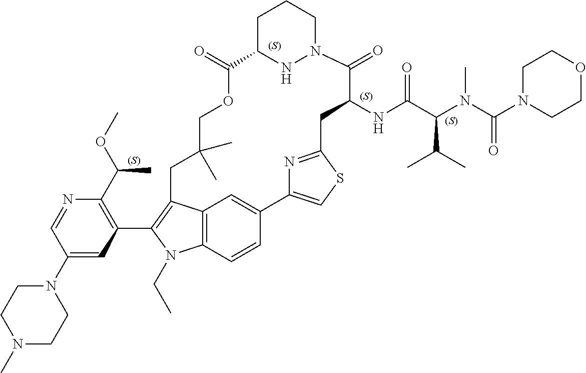 |
| AA556 | 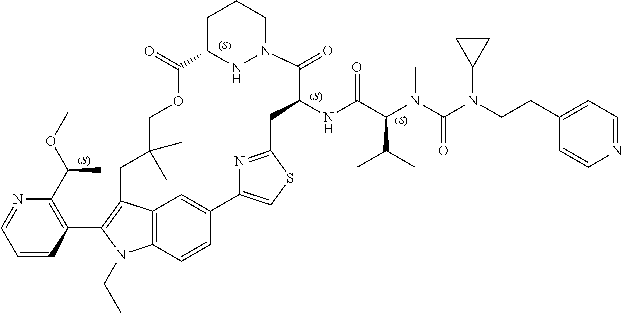 |
| AA557 | 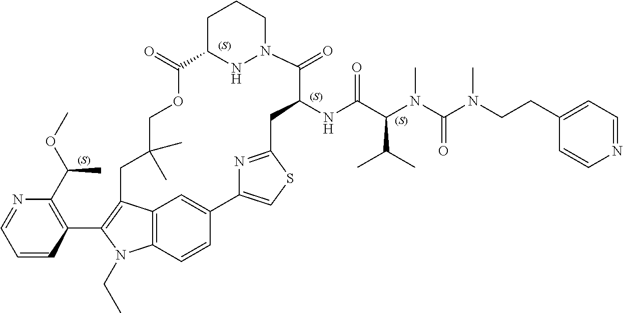 |
| AA558 | 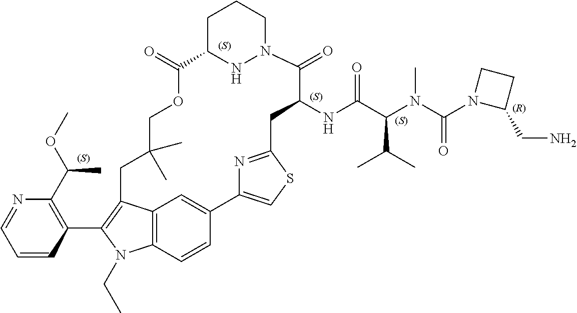 |
| AA559 | 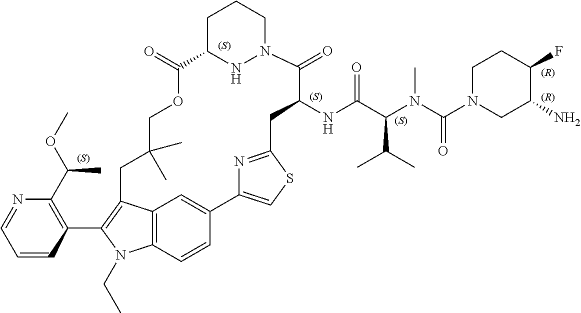 |
| AA560 | 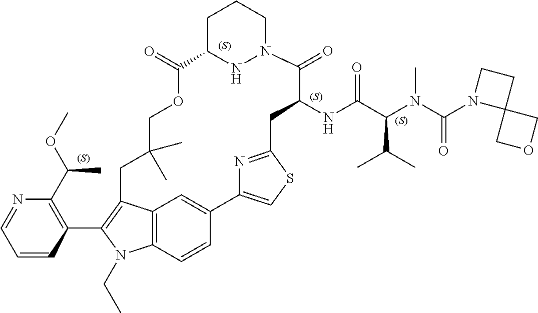 |
| AA561 | 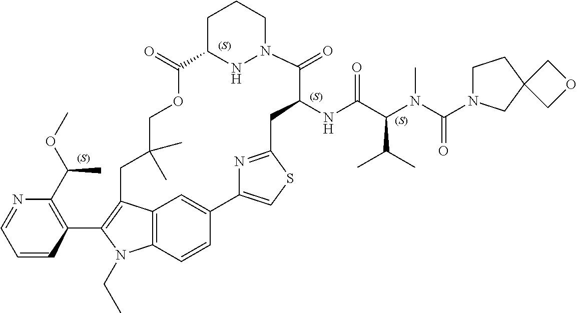 |
| AA562 | 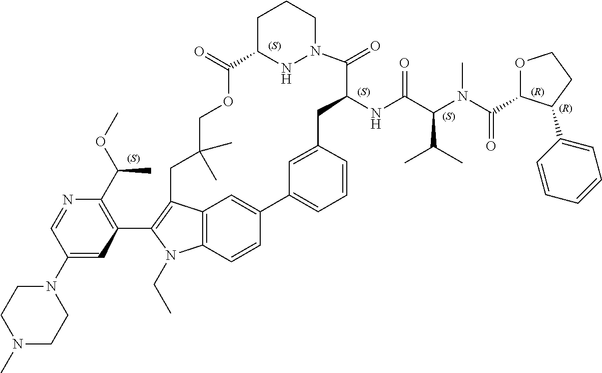 |
| AA563 | 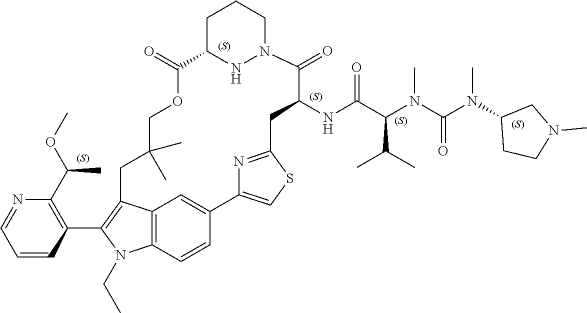 |
| AA564 | 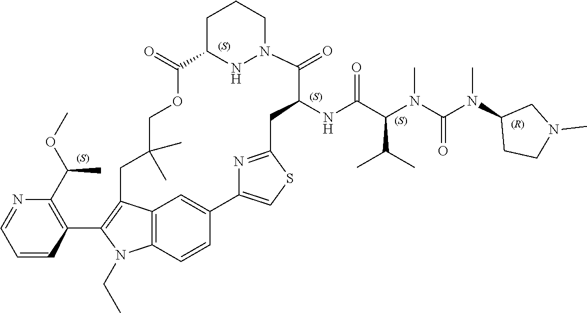 |
| AA565 | 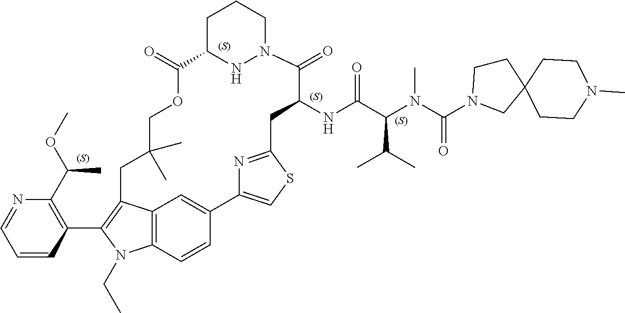 |
| AA566 | 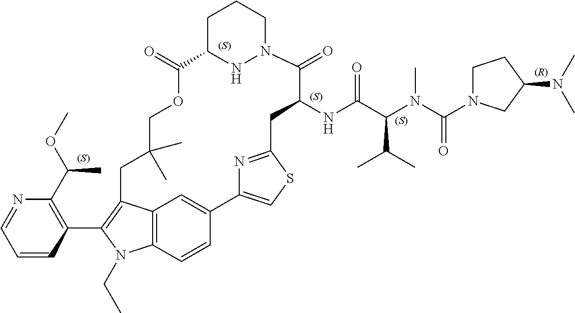 |
| AA567 | 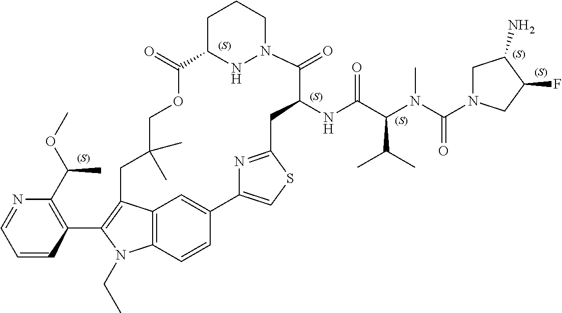 |
| AA568 | 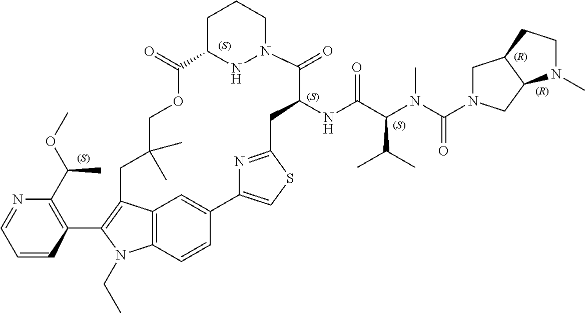 |
| AA569 | 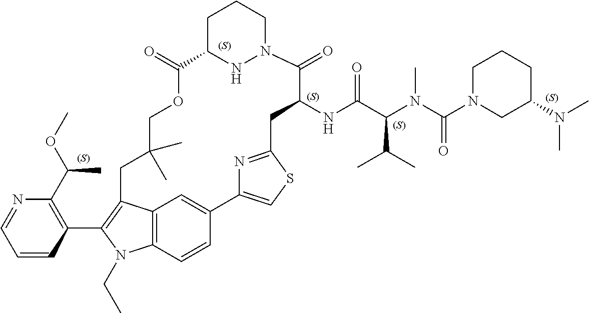 |
| AA570 | 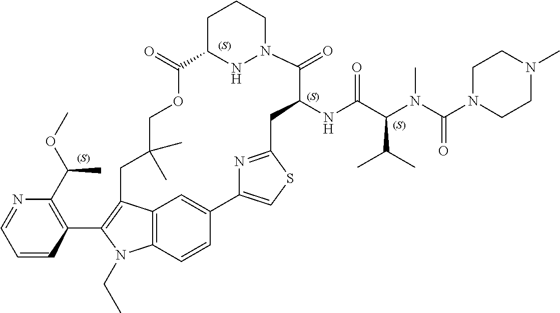 |
| AA571 | 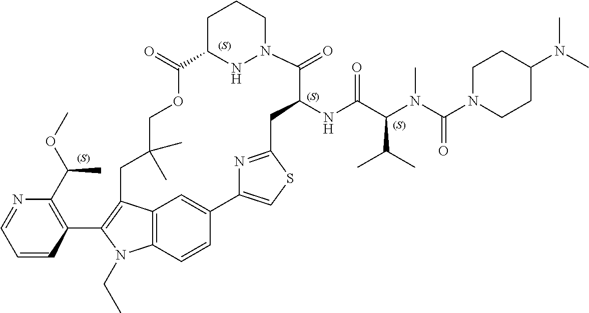 |
| AA572 | 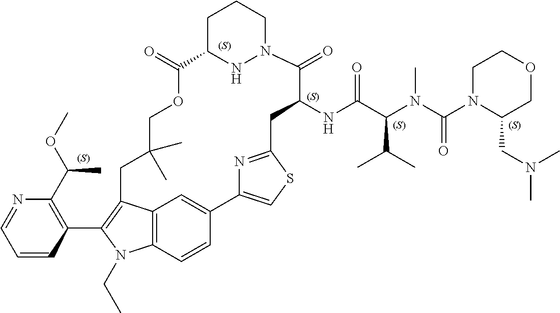 |
| AA573 | 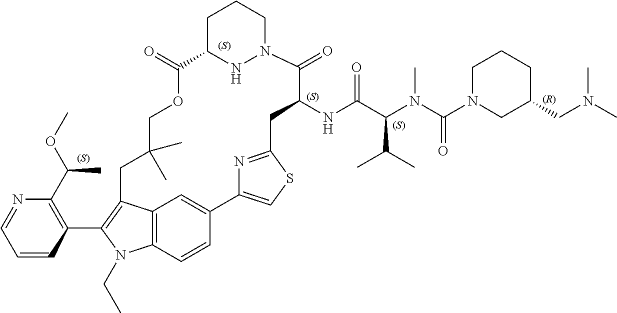 |
| AA574 | 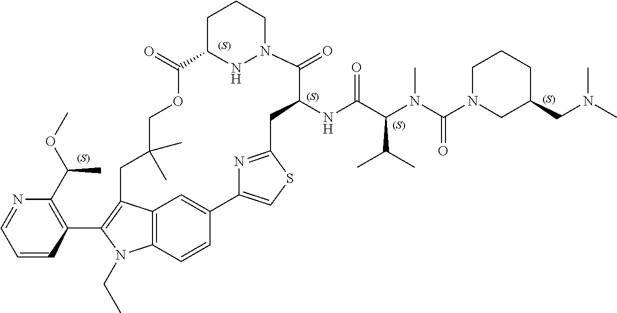 |
| AA575 | 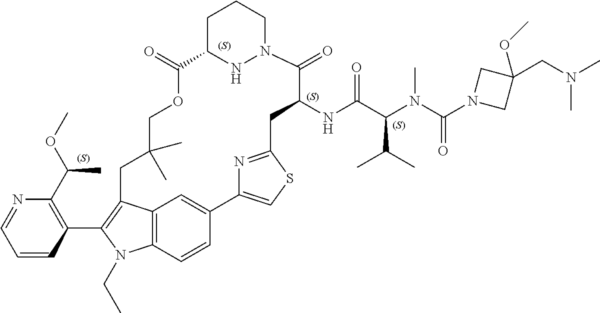 |
| AA576 | 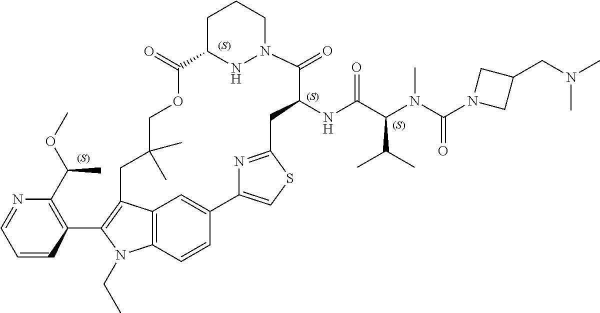 |
| AA577 | 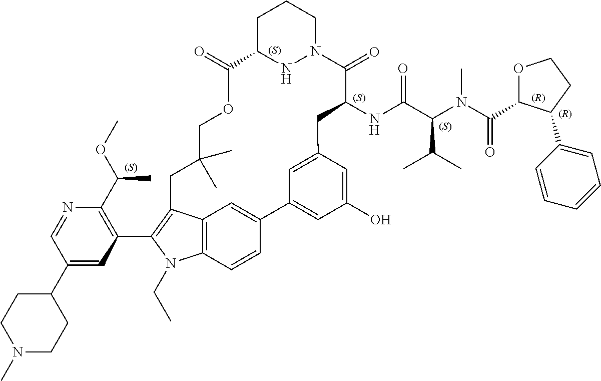 |
| AA578 | 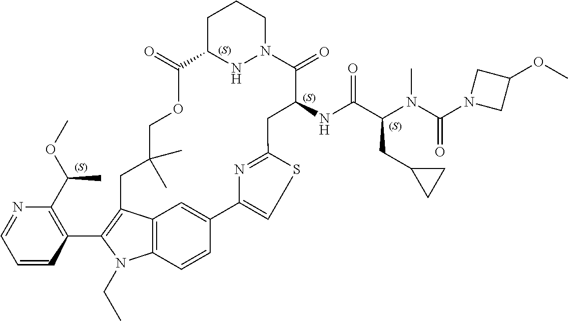 |
| AA579 | 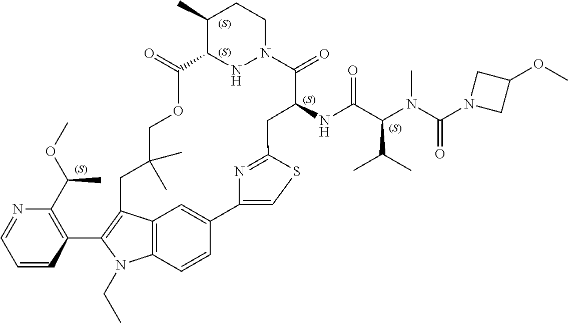 |
| AA580 | 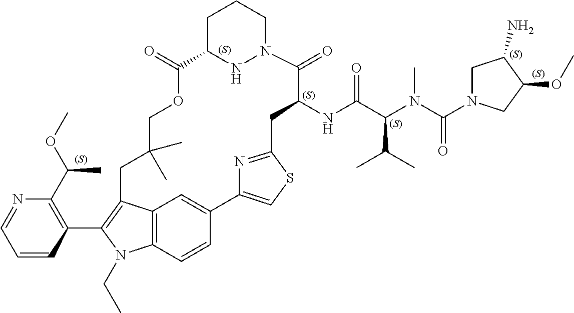 |
| AA581 | 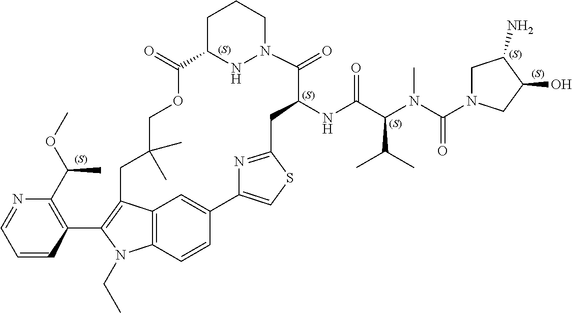 |
| AA582 | 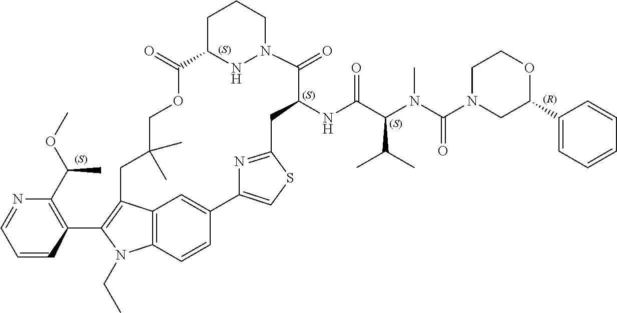 |
| AA583 | 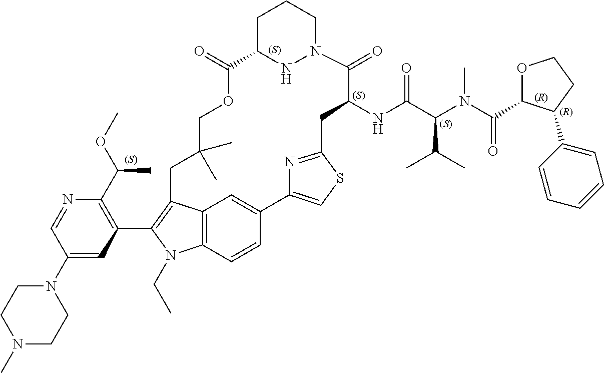 |
| AA584 | 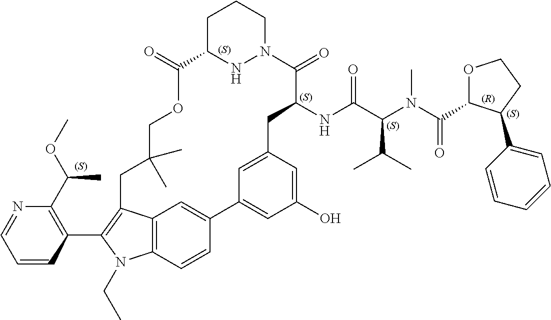 |
| AA585 | 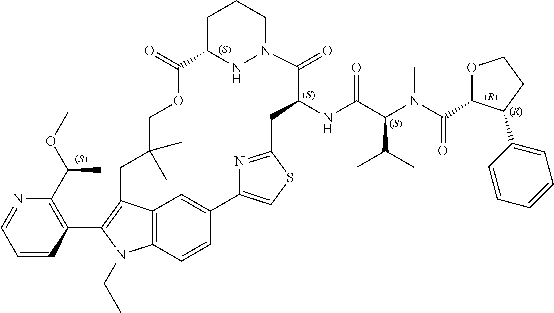 |
| AA586 | 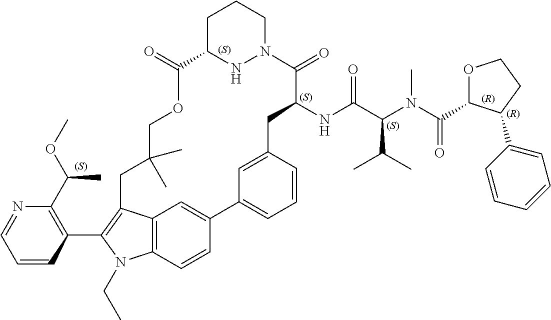 |
| AA587 | 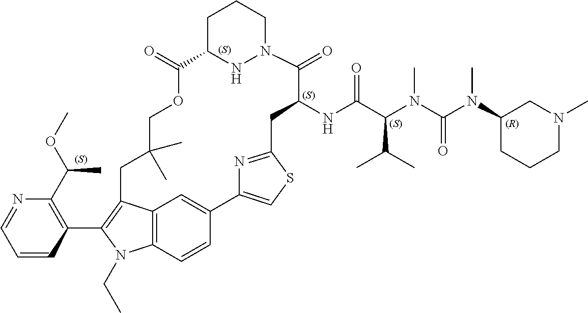 |
| AA588 | 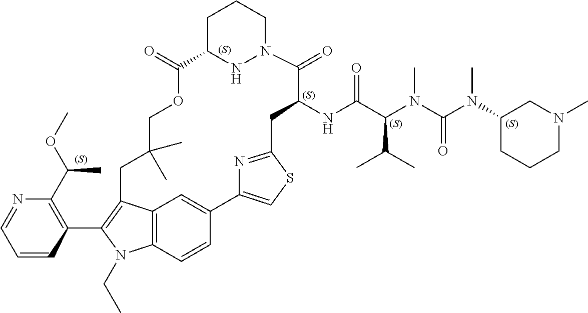 |
| AA589 | 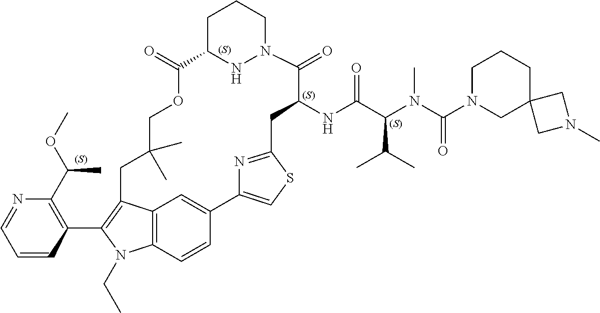 |
| AA590 | 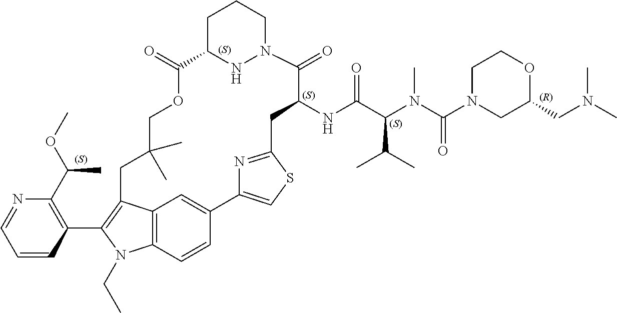 |
| AA591 | 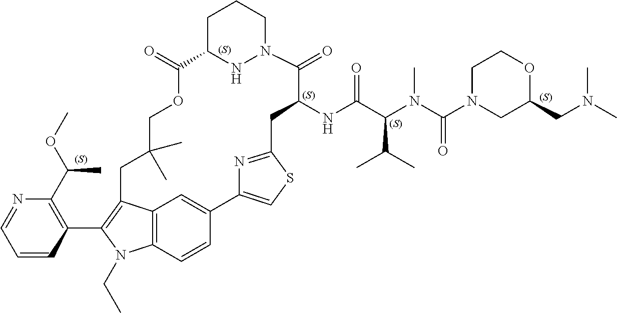 |
| AA592 | 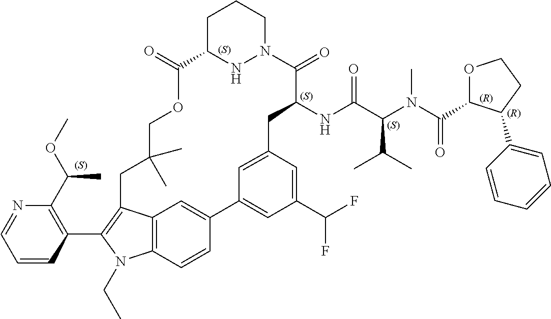 |
| AA593 | 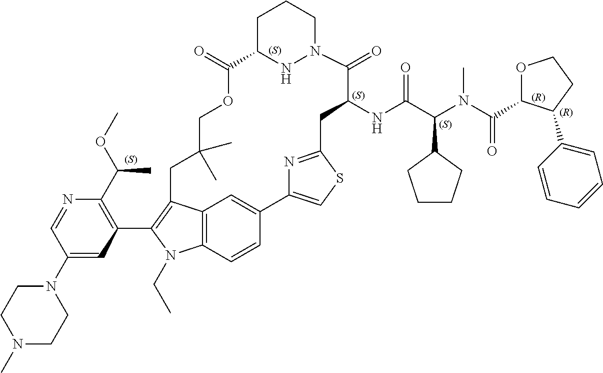 |
| AA594 | 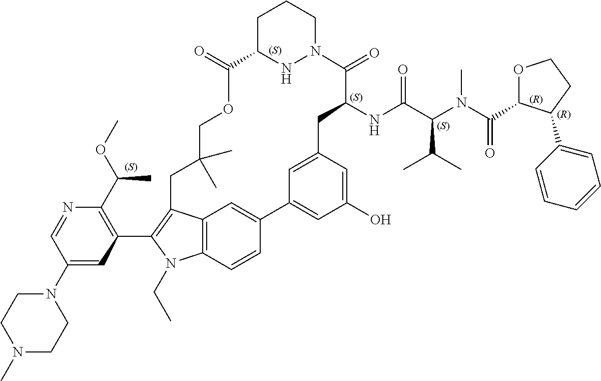 |
| AA595 | 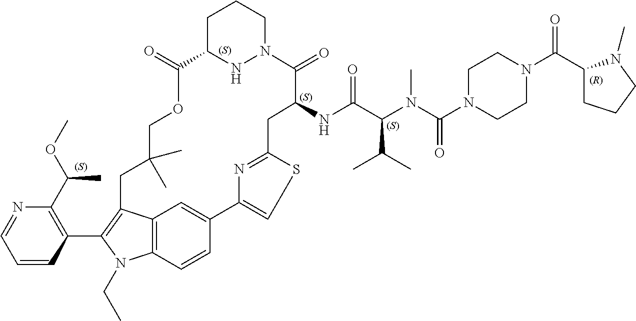 |
| AA596 | 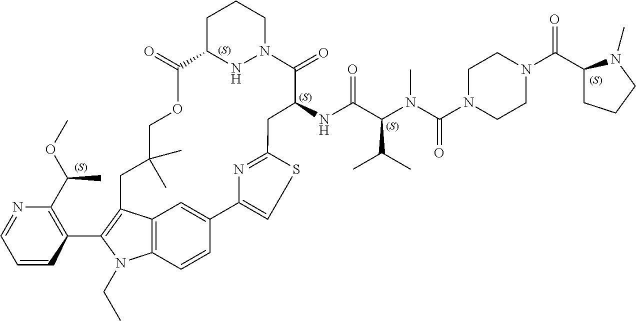 |
| AA597 | 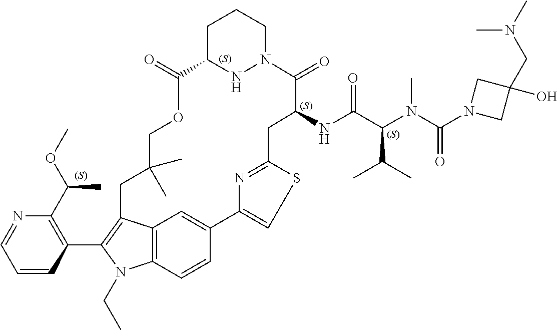 |
| AA598 | 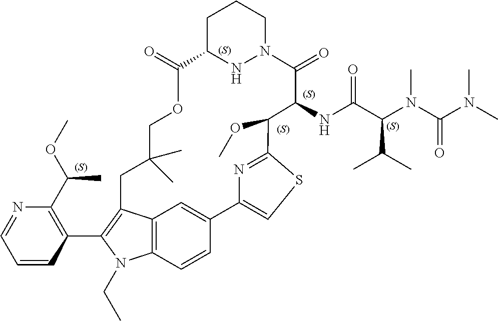 |
| AA599 | 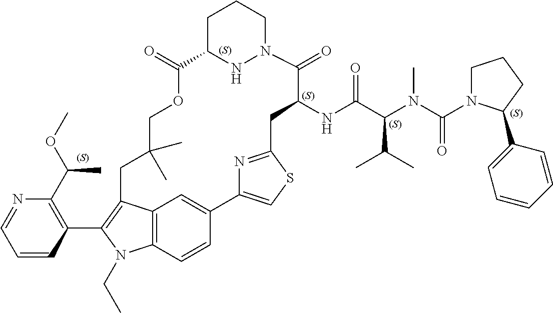 |
| AA600 | 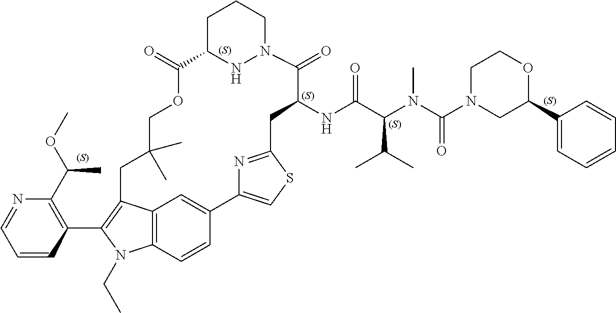 |
| AA601 | 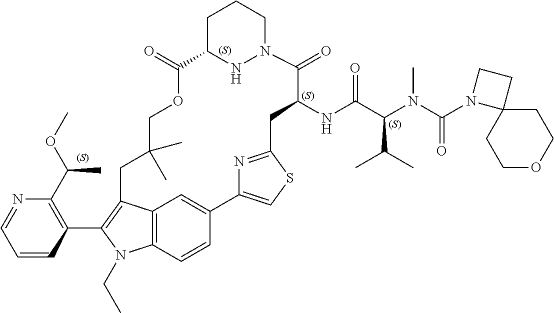 |
| AA602 | 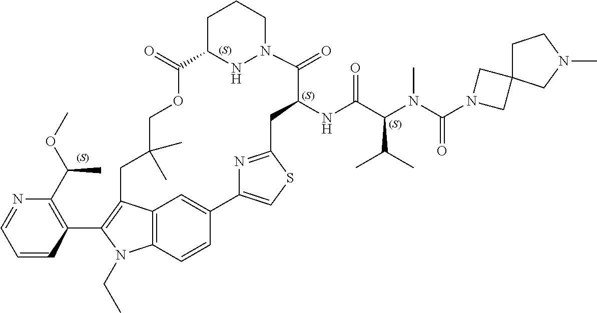 |
| AA603 | 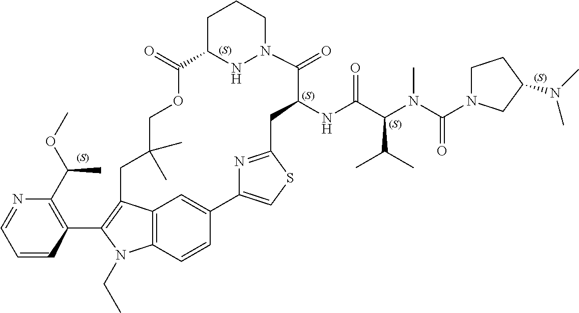 |
| AA604 | 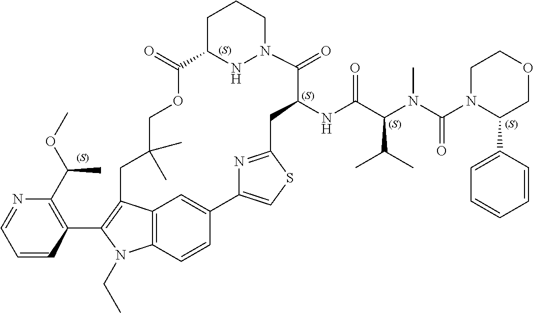 |
| AA605 | 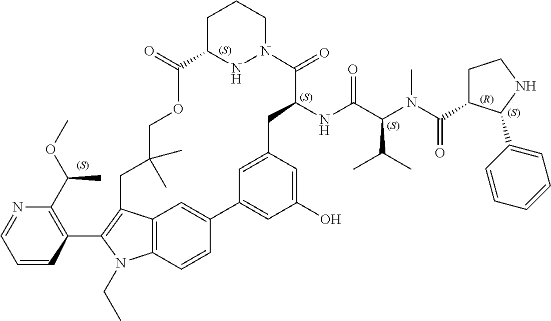 |
| AA606 | 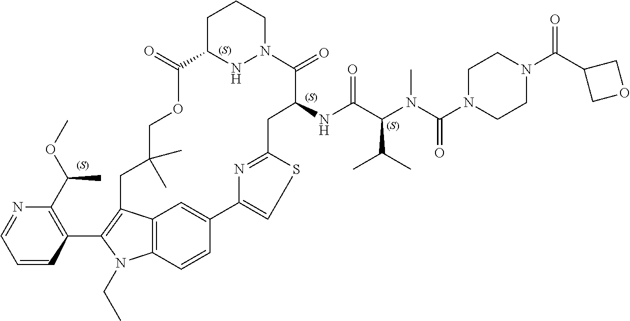 |
| AA607 | 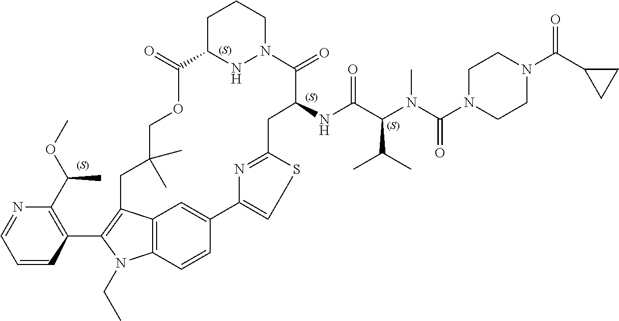 |
| AA608 | 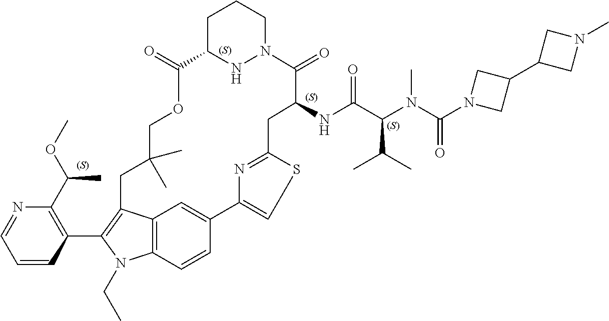 |
| AA609 | 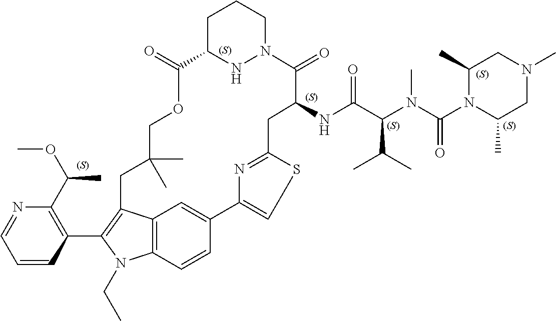 |
| AA610 | 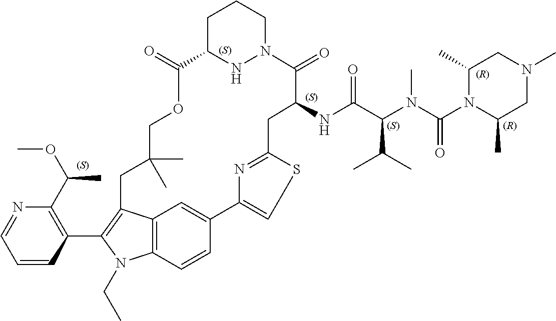 |
| AA611 | 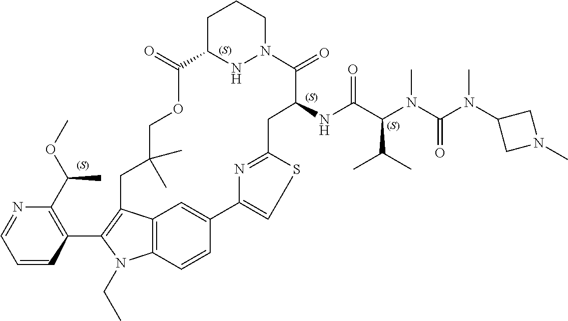 |
| AA612 | 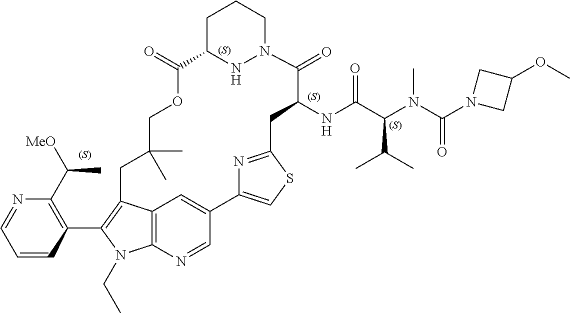 |
| AA613 | 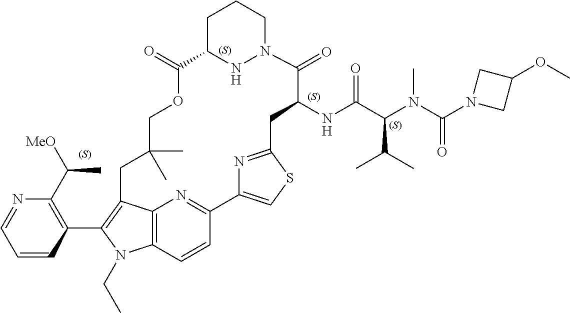 |
| AA614 | 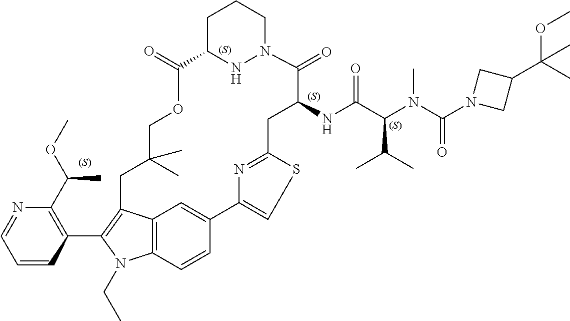 |
| AA615 | 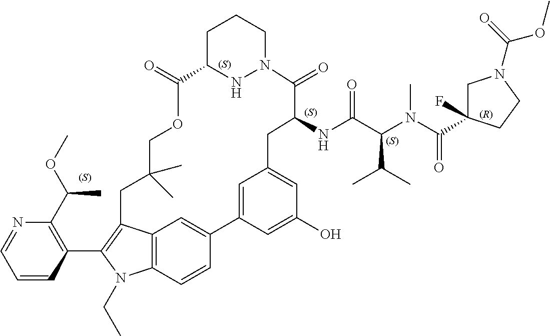 |
| AA616 | 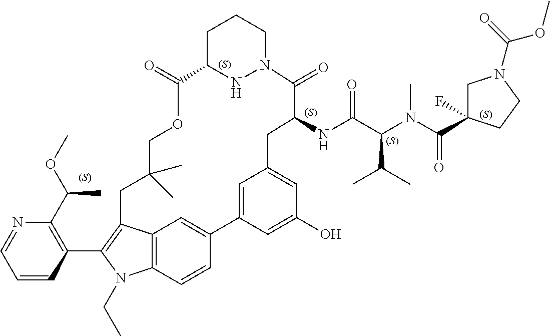 |

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

In some embodiments, a compound of Table A2 is provided, or a pharmaceutically acceptable salt thereof. In some embodiments, the RAS(ON) inhibitor is selected from Table A2, or a pharmaceutically acceptable salt or atropisomer thereof.

TABLE A2
Certain Compounds of the Present Invention

| Ex# | Structure |
|---|---|
| AB4 | 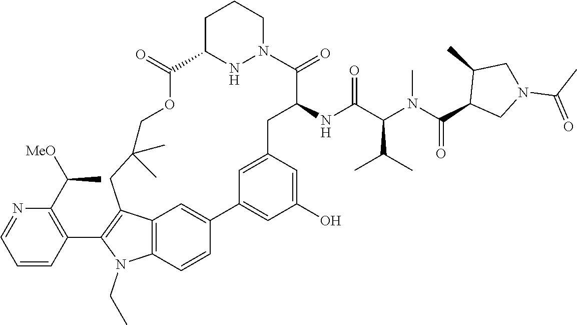 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| AB5 | 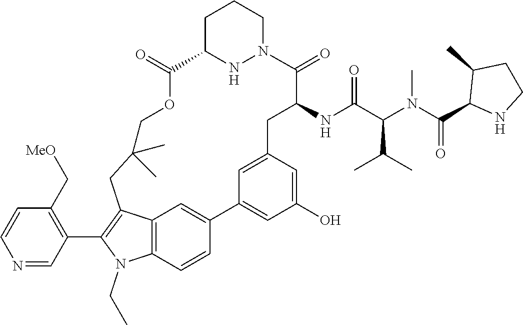 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| AB6 | 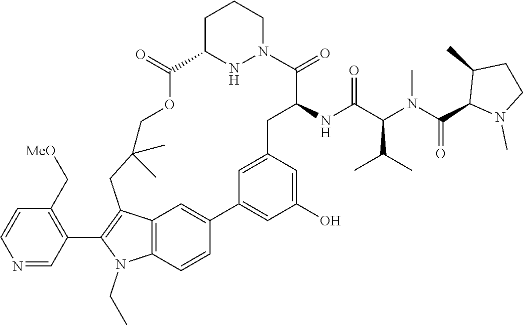 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| AB8 | 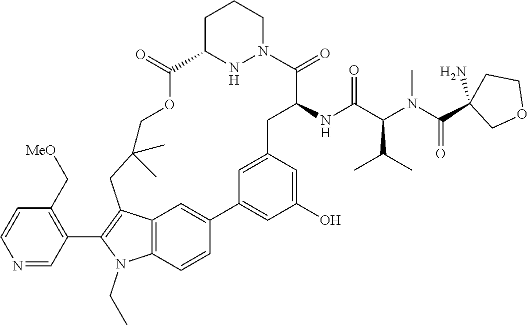 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| AB9 | 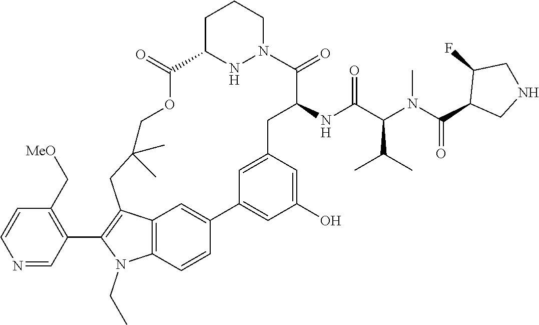 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| AB12 | 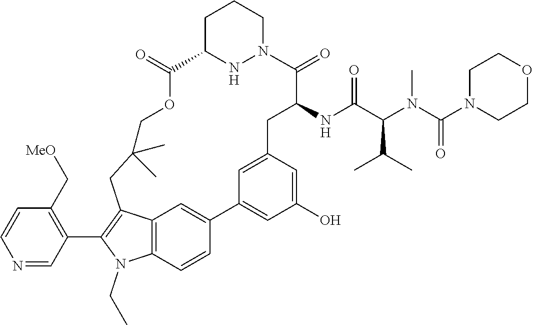 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AB13 | 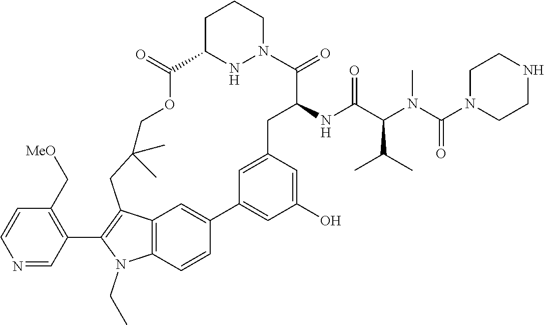 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AB19 | 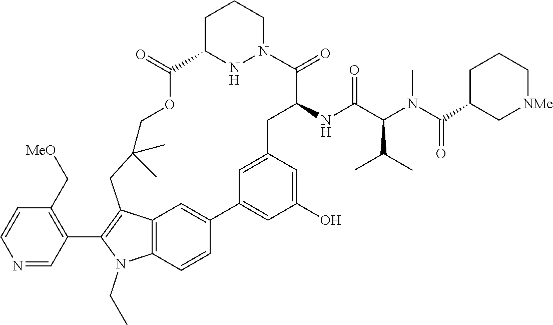 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| AB44 | 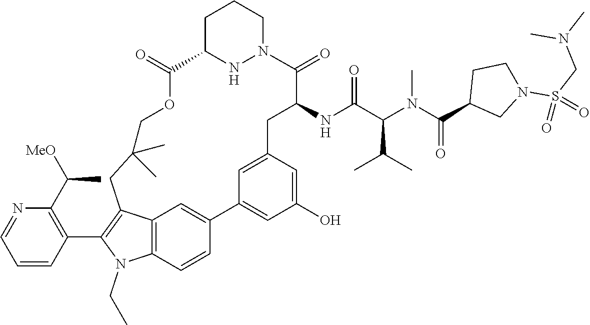 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)CN(C)C)C1)C(=O)N1CCC[C@H](N1)C(=O)OCC(C)(C)C2] |
| AB47 | 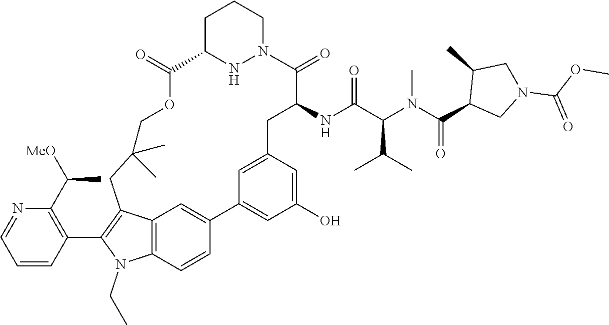 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2] |
| AB57 | 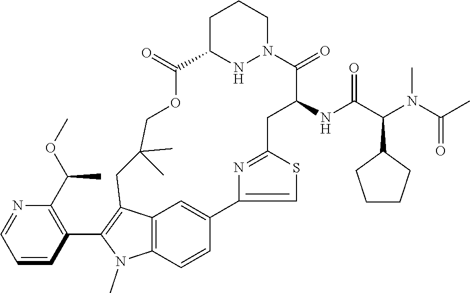 |
| AB58 | 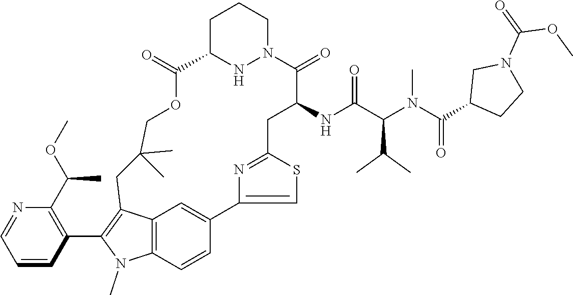 |
| AB59 | 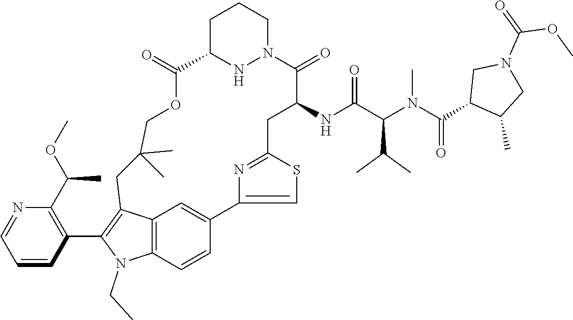 |
| AB60 | 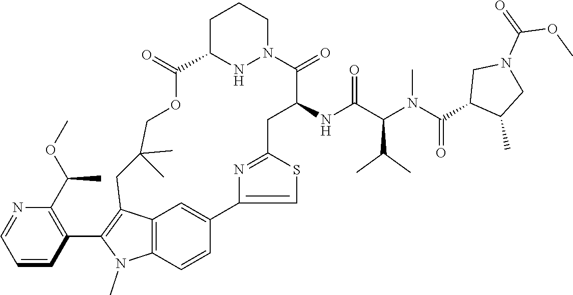 |
| AB61 | 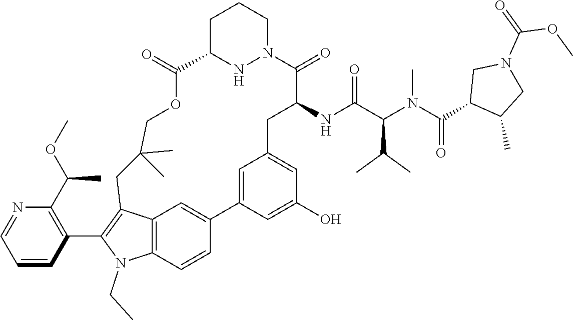 |
| AB66 | 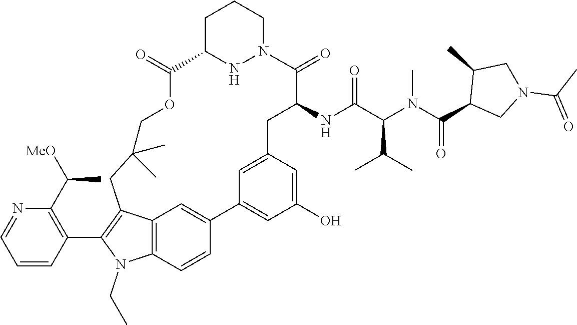 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| AB67 | 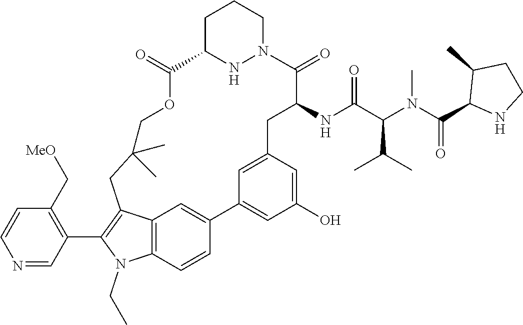 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| AB69 | 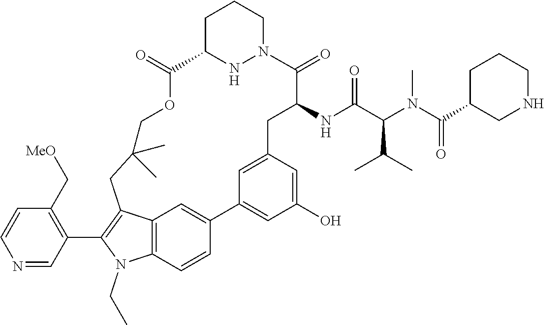 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCNC1)C(=O)N1CCC[C@H](N1)C(=O)OCC(C)(C)C2] |
| AB71 | 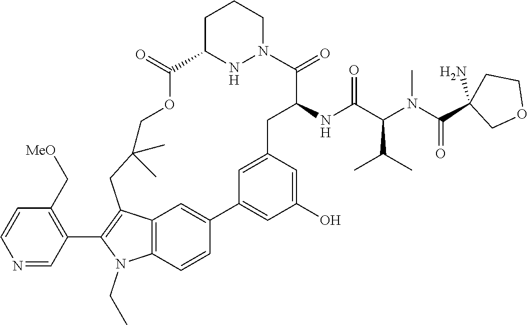 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| AB73 | 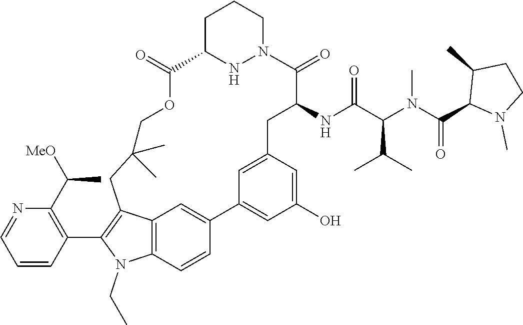 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| AB74 | 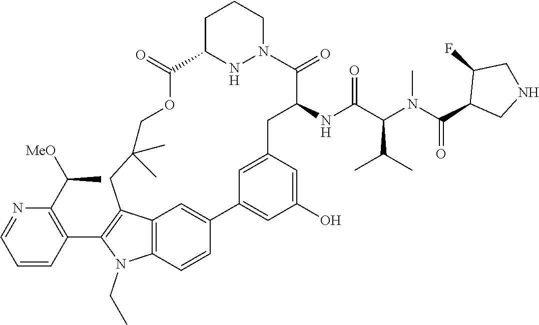 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| AB80 | 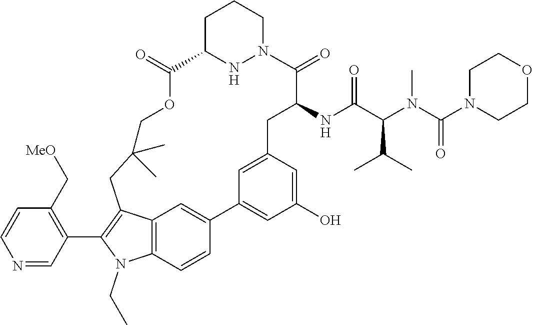 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AB81 | 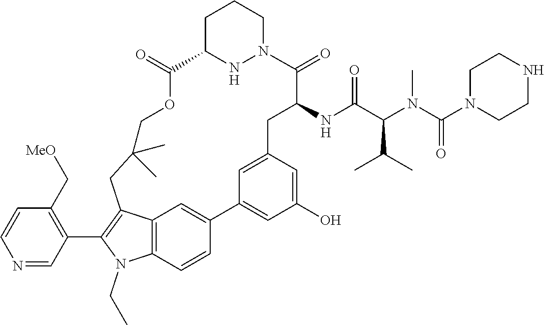 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AB94 | 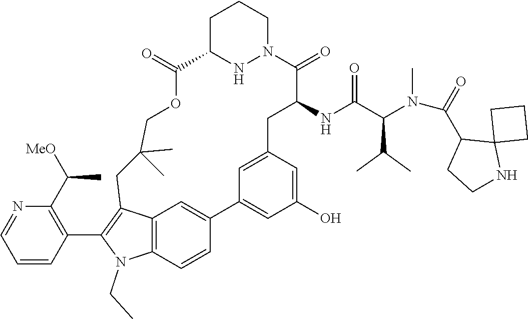 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CCC3)C(=O)N1CCC[C@H](N1)C(=O)OCC(C)(C)C2] |
| AB95 | 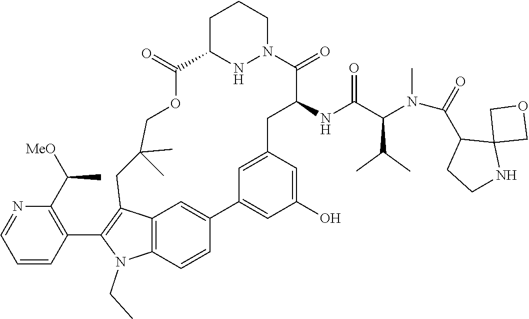 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13COC3)C(=O)N1CCC[C@H](N1)C(=O)OCC(C)(C)C2] |
| AB96 | 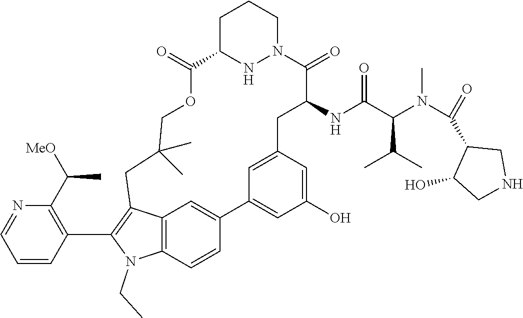 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| AB97 | 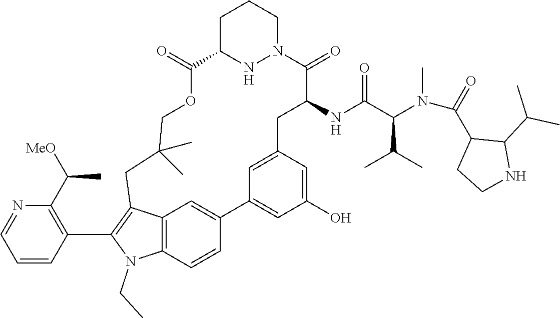 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC1C(C)C)C(=O)N1CCC[C@H](N1)C(=O)OCC(C)(C)C2] |
| AB99 | 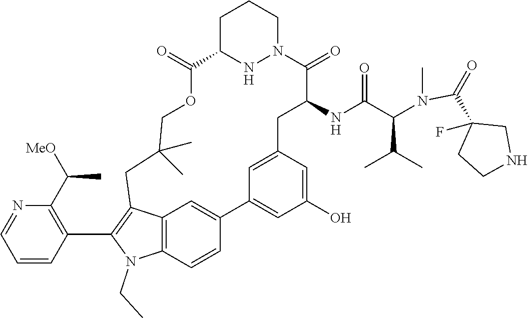 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(F)CCNC1)C(=O)N1CCC[C@H](N1)C(=O)OCC(C)(C)C2] |
| AB100 | 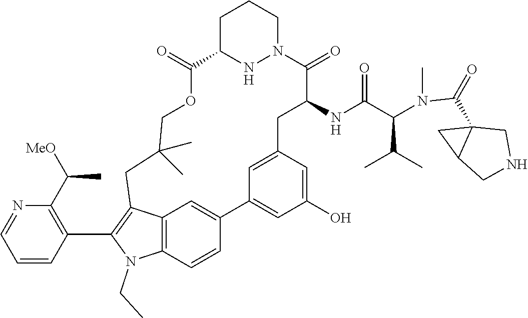 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@]13CNCC1C3)C(=O)N1CCC[C@H](N1)C(=O)OCC(C)(C)C2] |
| AB104 | 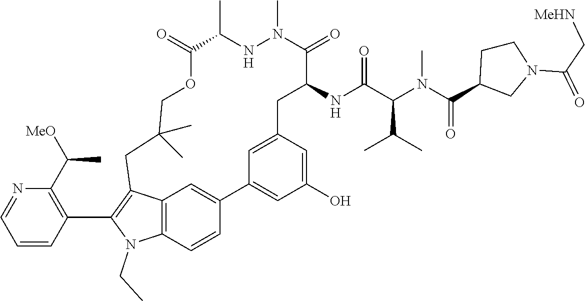 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N(C)N[C@@H](C)C(=O)OCC(C)(C)C2] |
| AB106 | 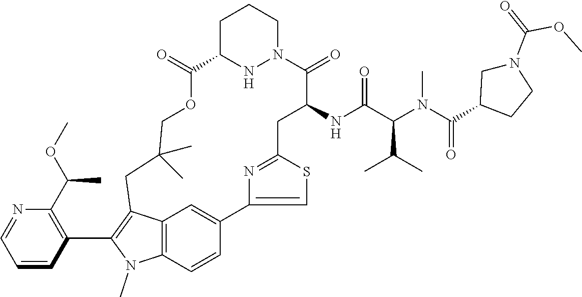 |
| AB107 | 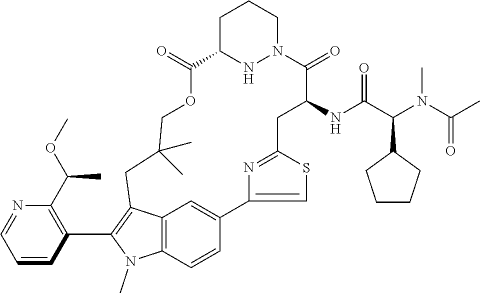 |
| AB109 | 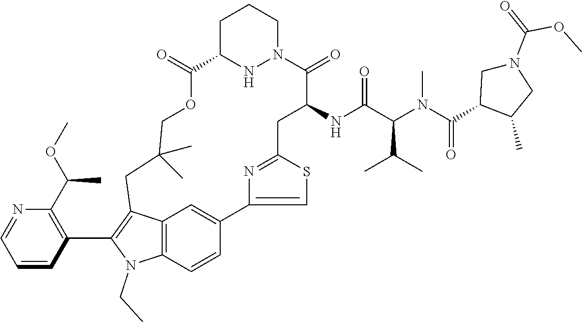 |
| AB110 | 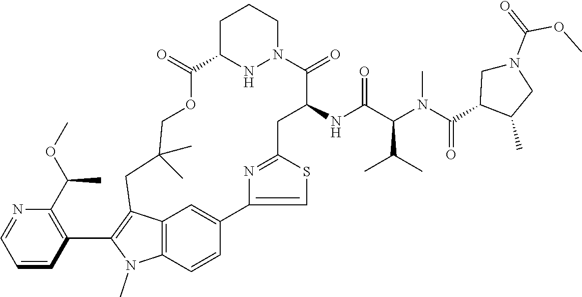 |
| AB111 | 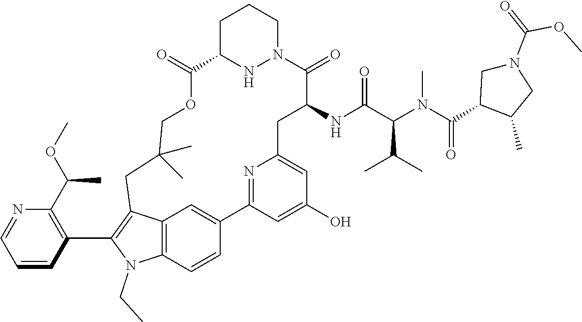 |
| AB112 | 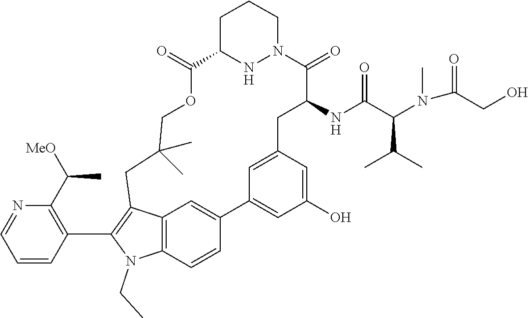 [SMILES: CCn1c(-c2cccnc2[C@H](C)OC)c2c3cc(ccc31)-c1cc(O)cc(c1)C[C@H](NC(=O)[C@H](C(C)C)N(C)C(=O)CO)C(=O)N1CCC[C@H](N1)C(=O)OCC(C)(C)C2] |
| AB113 | 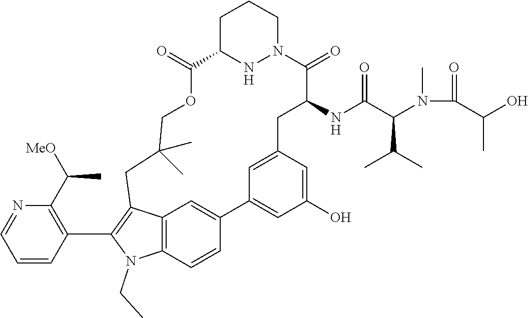 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(C)O)C(=O)N1CCC[C@H](N1)C(=O)OCC(C)(C)C2] |
| AB114 | 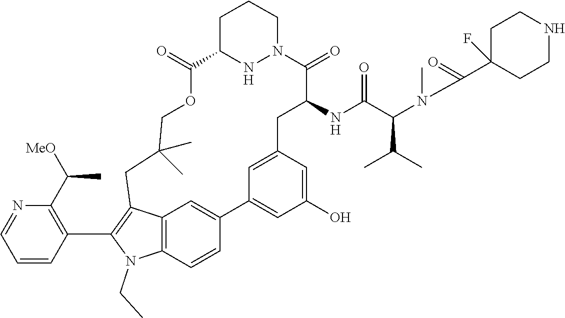 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(F)CCNCC1)C(=O)N1CCC[C@H](N1)C(=O)OCC(C)(C)C2] |
| AB117 | 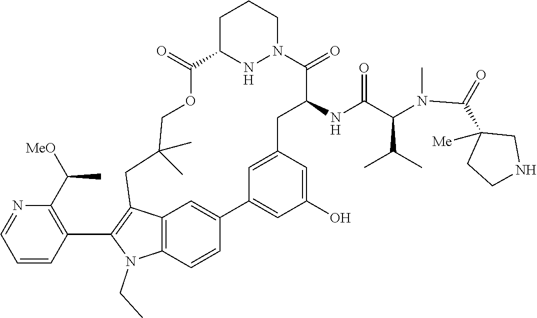 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(C)CCNC1)C(=O)N1CCC[C@H](N1)C(=O)OCC(C)(C)C2] |
| AB119 | 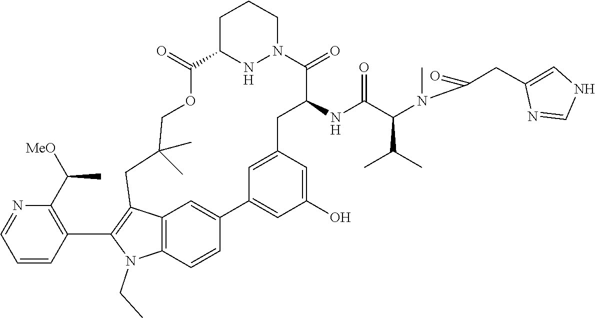 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1c[nH]cn1)C(=O)N1CCC[C@H](N1)C(=O)OCC(C)(C)C2] |
| AB122 | 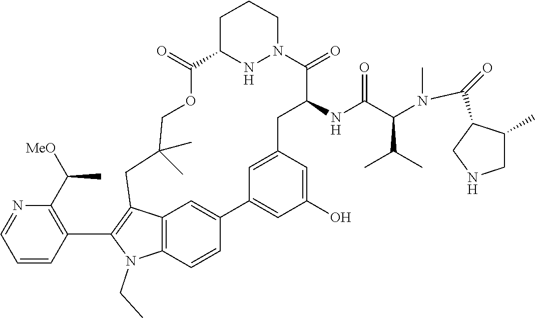 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CNC[C@H]1C)C(=O)N1CCC[C@H](N1)C(=O)OCC(C)(C)C2] |
| AB123 | 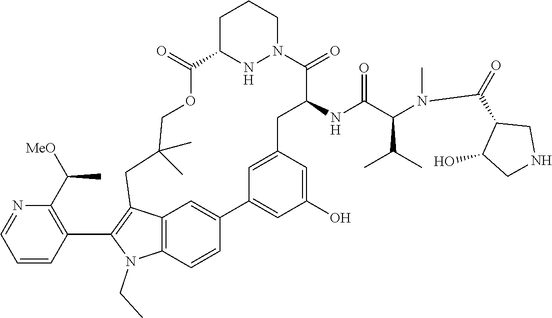 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| AB124 | 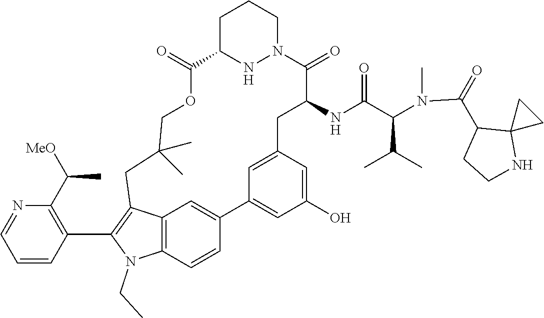 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CC3)C(=O)N1CCC[C@H](N1)C(=O)OCC(C)(C)C2] |
| AB126 | 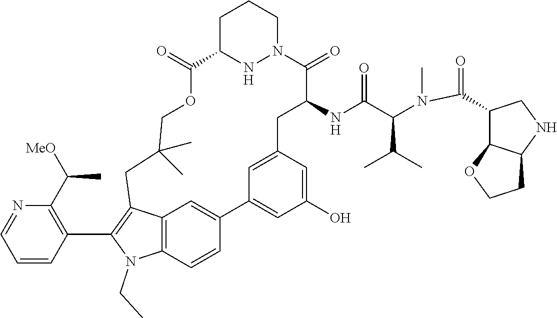 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN[C@H]3CCO[C@H]31)C(=O)N1CCC[C@H](N1)C(=O)OCC(C)(C)C2] |
| AB128 | 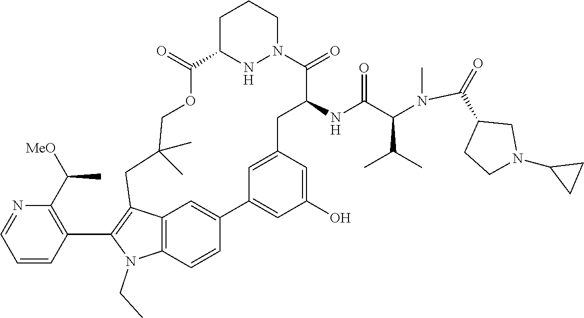 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C3CC3)C1)C(=O)N1CCC[C@H](N1)C(=O)OCC(C)(C)C2] |
| AB129 | 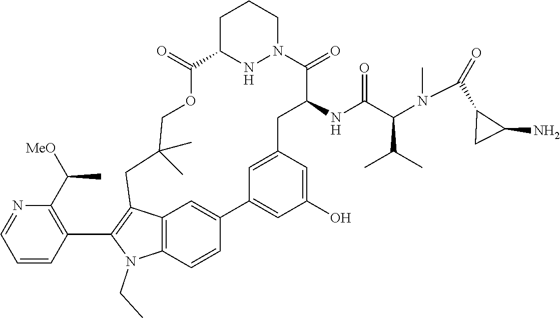 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H]1N)C(=O)N1CCC[C@H](N1)C(=O)OCC(C)(C)C2] |
| AB130 | 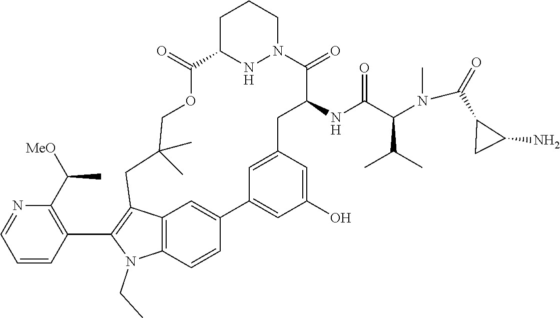 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@H]1N)C(=O)N1CCC[C@H](N1)C(=O)OCC(C)(C)C2] |
| AB133 | 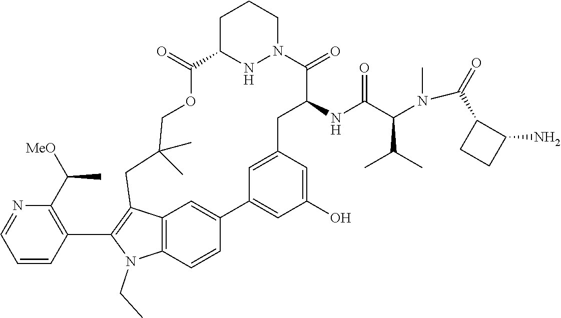 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CC[C@H]1N)C(=O)N1CCC[C@H](N1)C(=O)OCC(C)(C)C2] |
| AB134 | 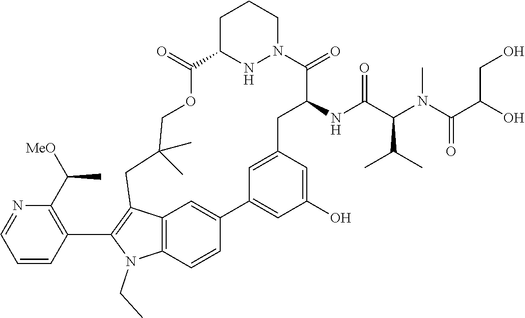 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(O)CO)C(=O)N1CCC[C@H](N1)C(=O)OCC(C)(C)C2] |
| AB135 | 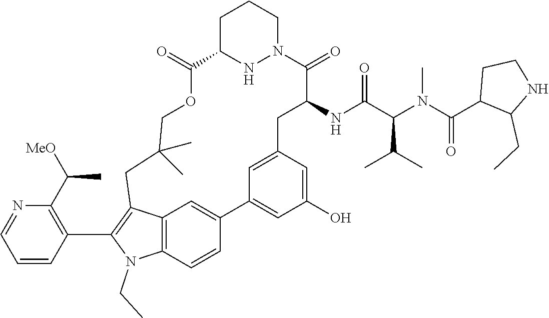 [SMILES: CCC1NCCC1C(=O)N(C)[C@H](C(=O)N[C@H]1Cc2cc(O)cc(c2)-c2ccc3c(c2)c(c(-c2cccnc2[C@H](C)OC)n3CC)CC(C)(C)COC(=O)[C@@H]2CCCN(N2)C1=O)C(C)C] |
| AB137 | 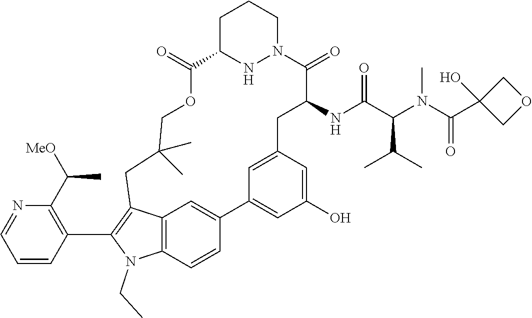 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| AB138 | 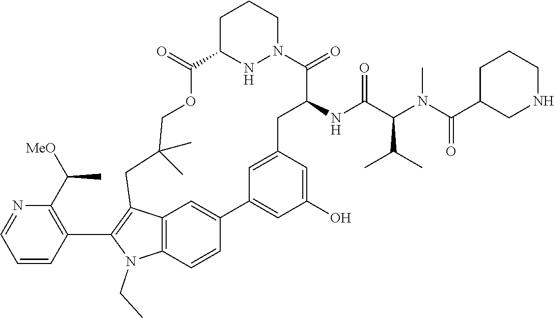 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCCNC1)C(=O)N1CCC[C@H](N1)C(=O)OCC(C)(C)C2] |
| AB139 | 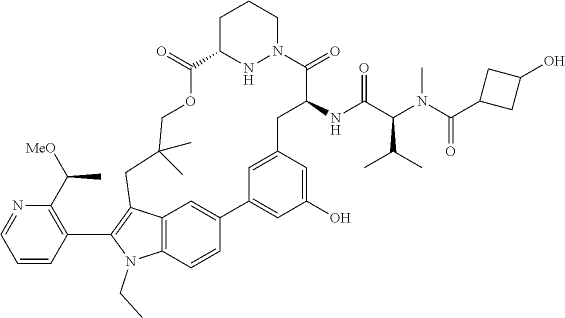 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC(O)C1)C(=O)N1CCC[C@H](N1)C(=O)OCC(C)(C)C2] |
| AB141 | 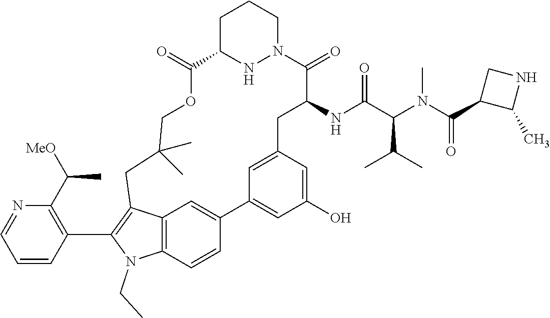 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CN[C@@H]1C)C(=O)N1CCC[C@H](N1)C(=O)OCC(C)(C)C2] |
| AB143 | 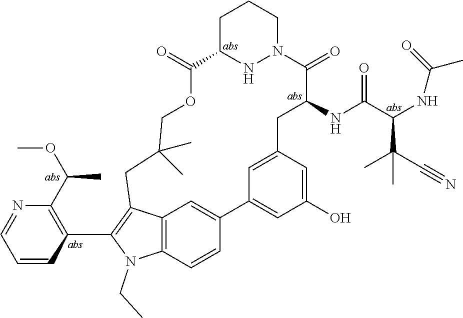 |
| AB144 | 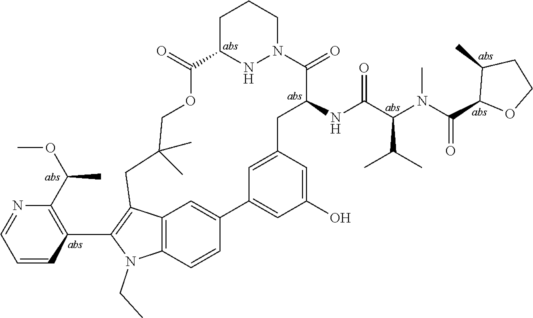 |
| AB145 | 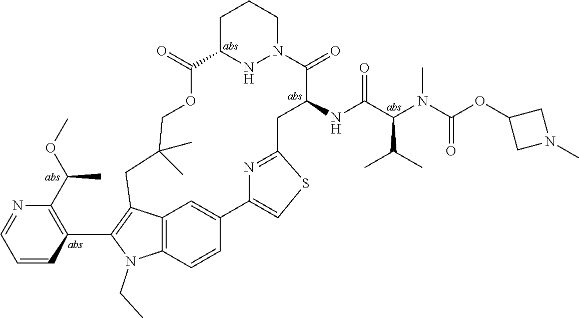 |
| AB146 | 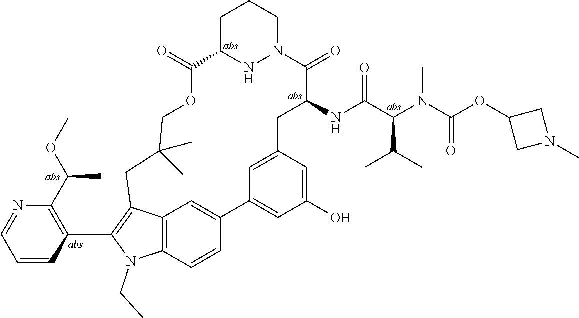 |
| AB147 | 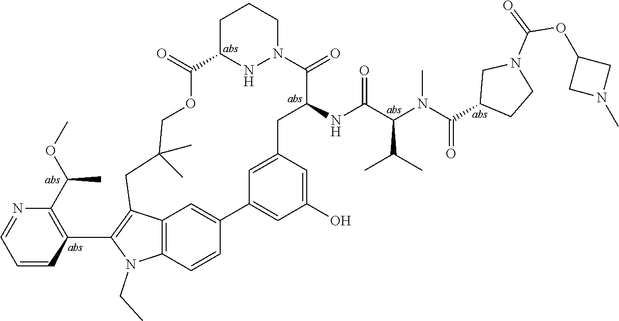 |
| AB148 | 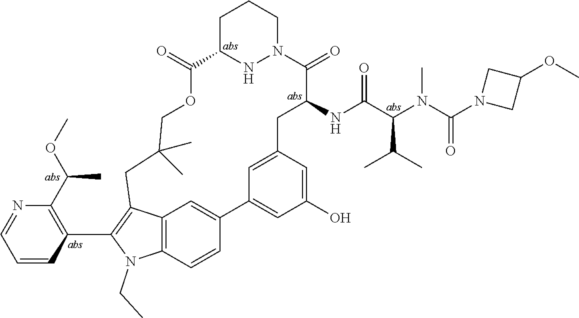 |
| AB149 | 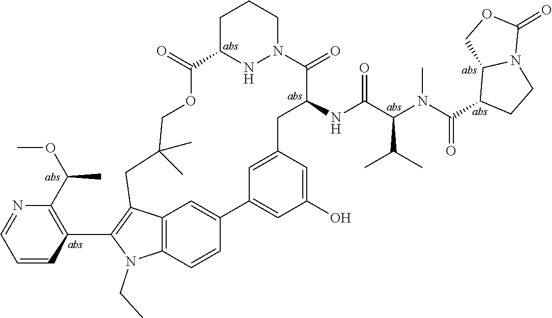 |
| AB150 | 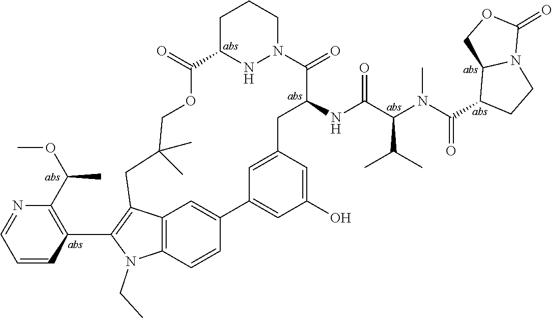 |
| AB151 | 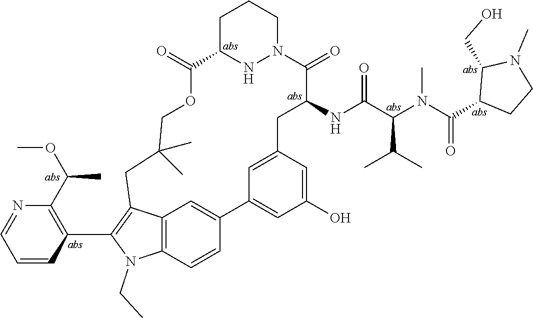 |
| AB152 | 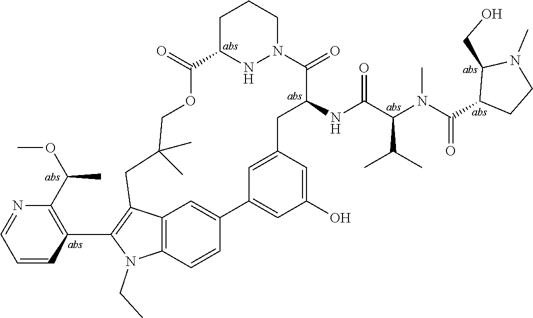 |
| AB153 | 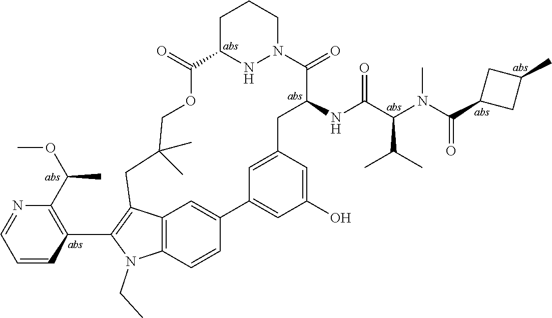 |
| AB154 | 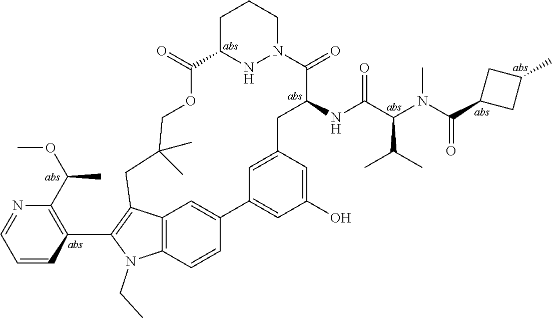 |
| AB155 | 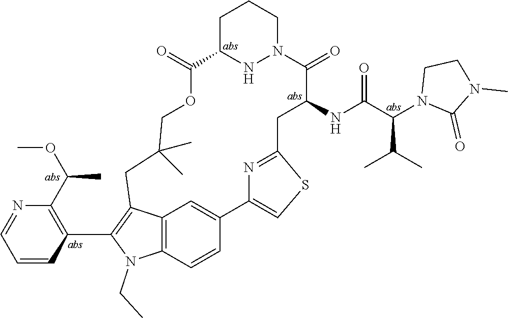 |
| AB156 | 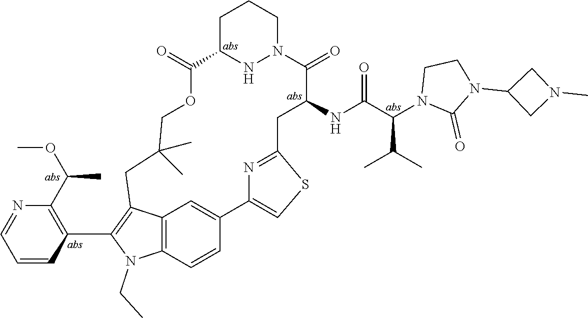 |
| AB157 | 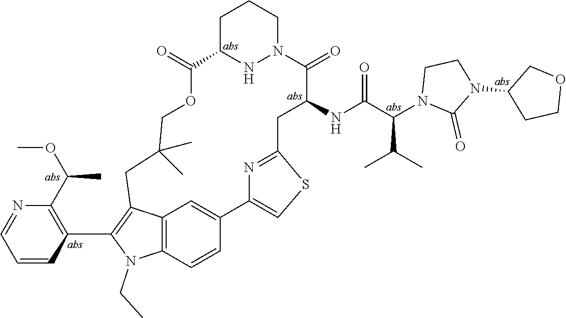 |
| AB158 | 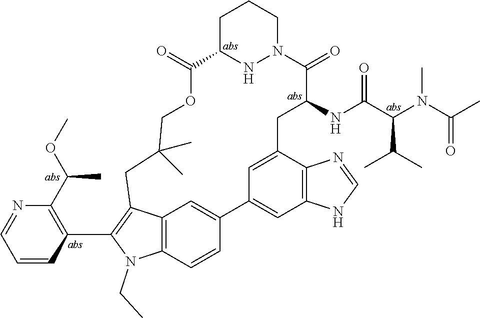 |
| AB159 | 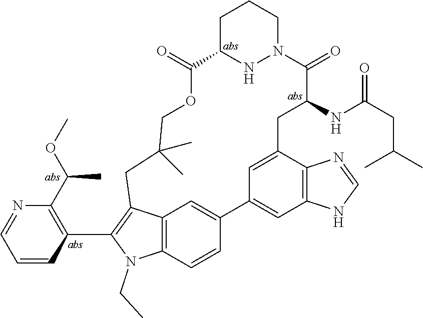 |
| AB160 | 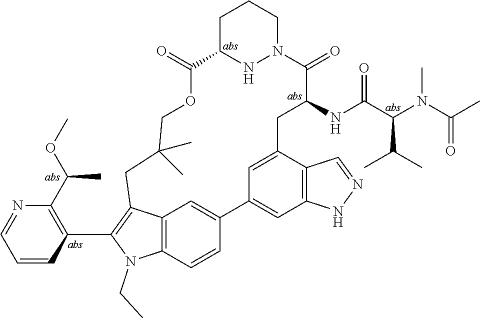 |
| AB161 | 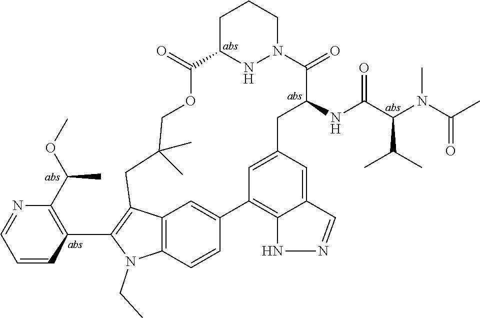 |
| AB162 | 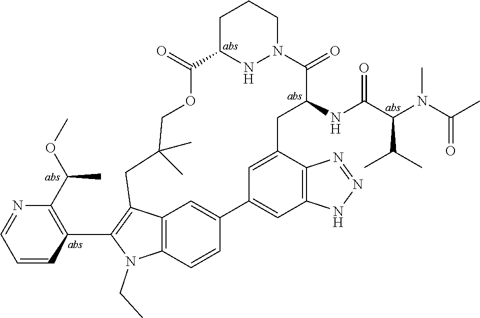 |
| AB163 | 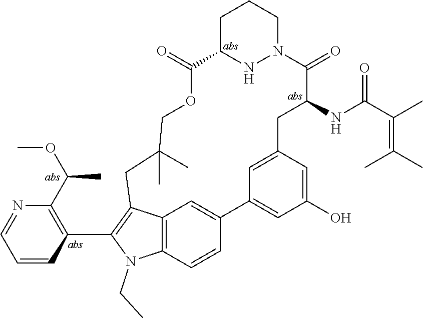 |
| AB164 | 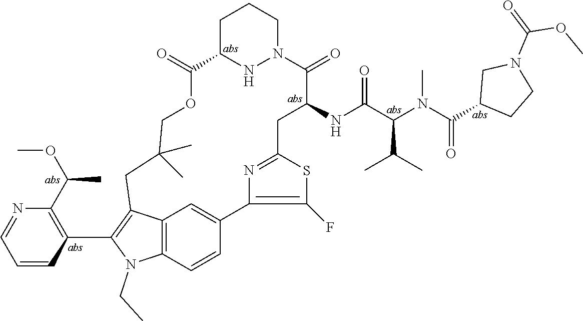 |
| AB165 | 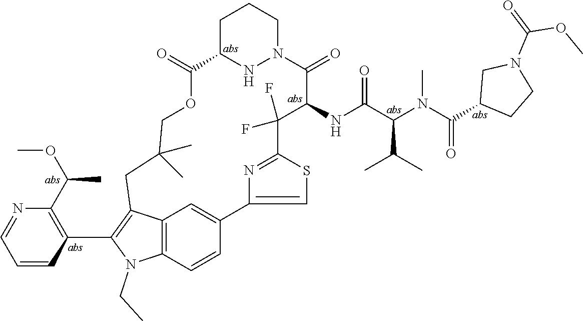 |
| AB166 | 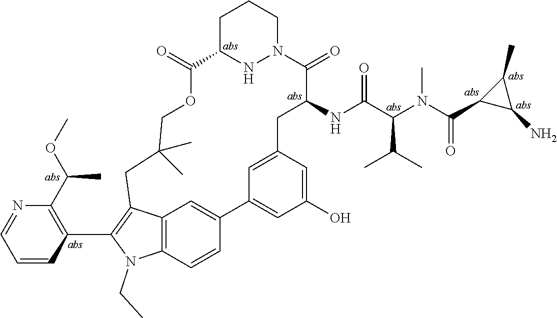 |
| AB167 | 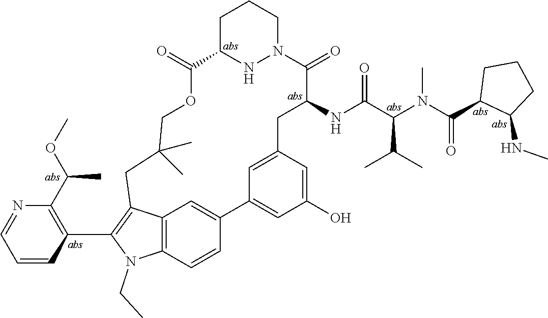 |
| AB168 | 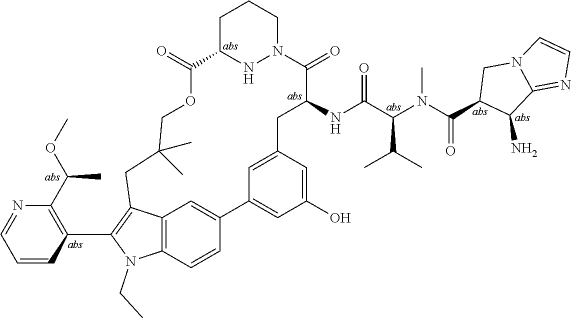 |
| AB169 | 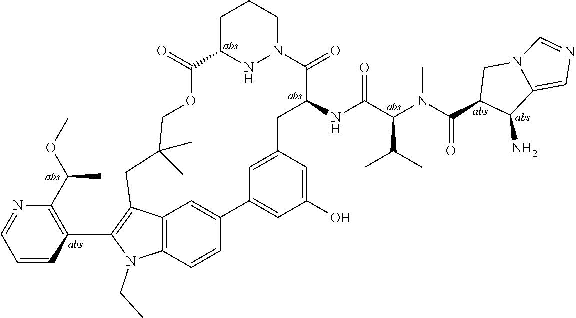 |
| AB170 | 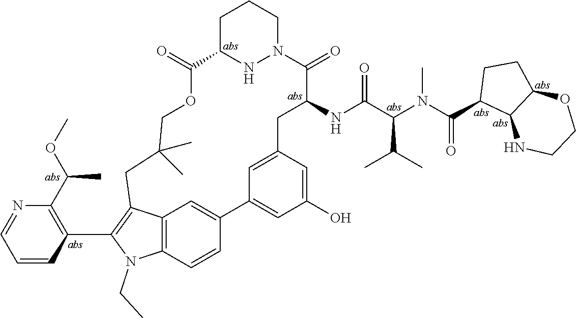 |
| AB171 | 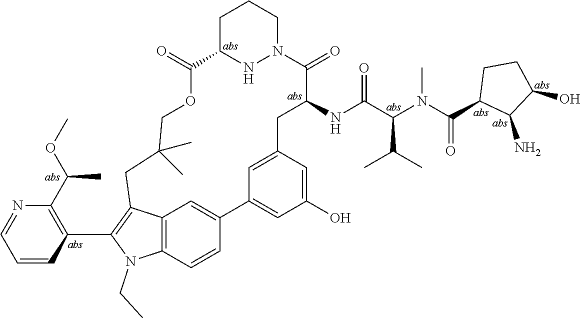 |
| AB172 | 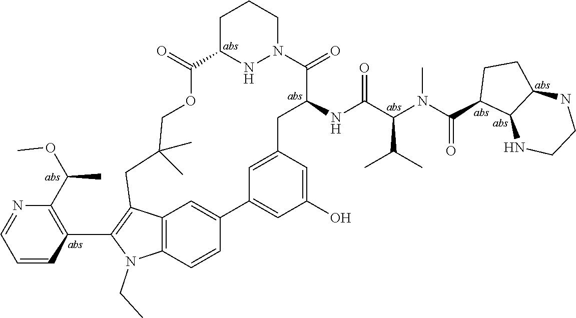 |
| AB173 | 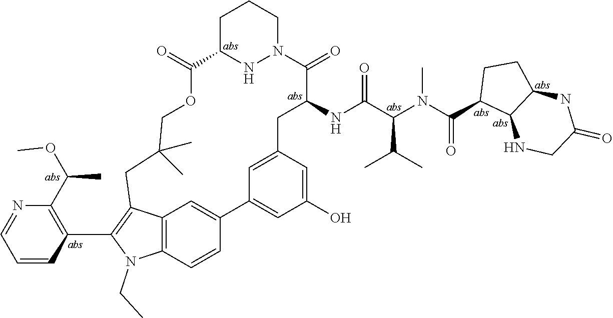 |
| AB174 | 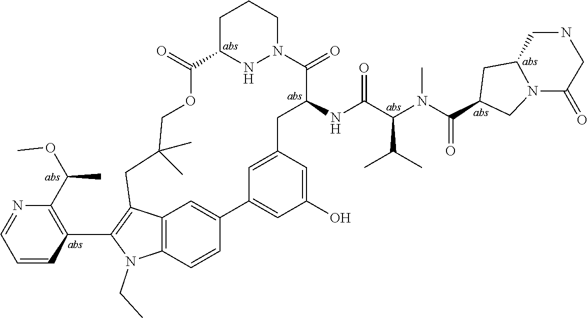 |
| AB175 | 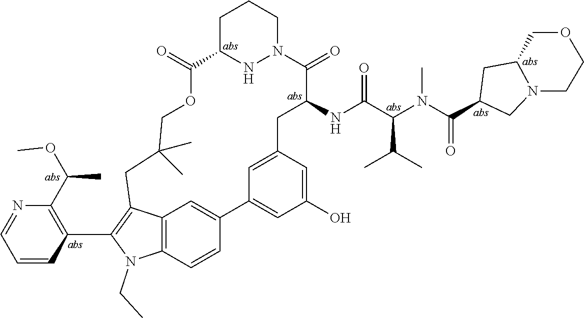 |
| AB176 | 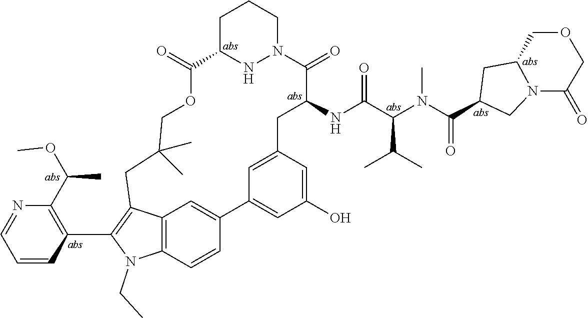 |
| AB177 | 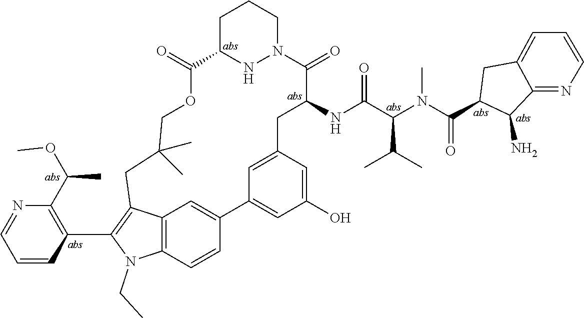 |
| AB178 | 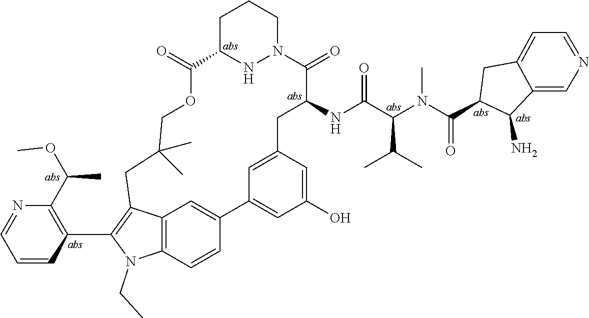 |
| AB179 | 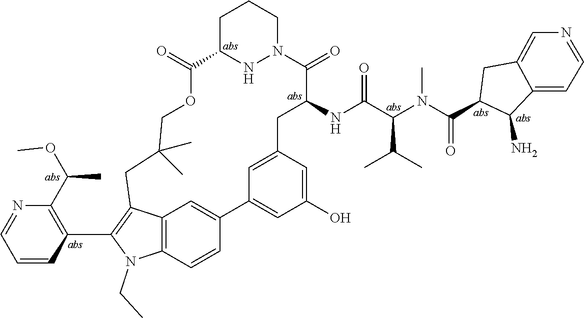 |
| AB180 | 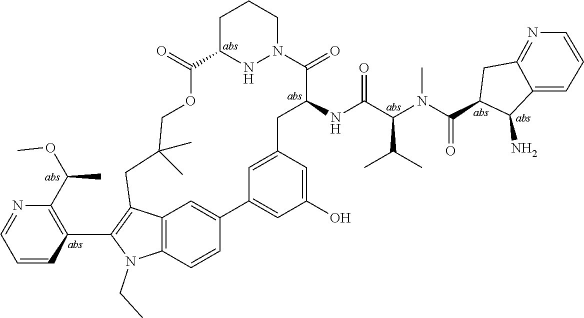 |
| AB181 | 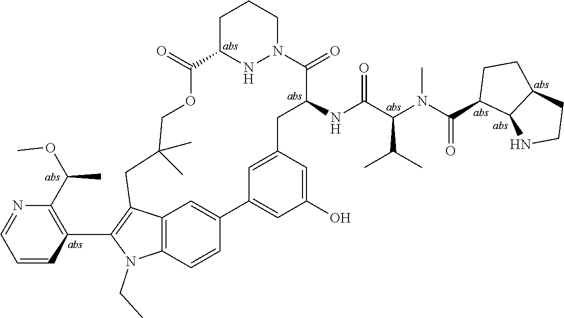 |
| AB182 | 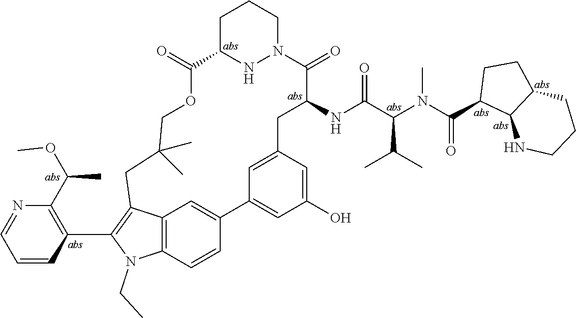 |
| AB183 | 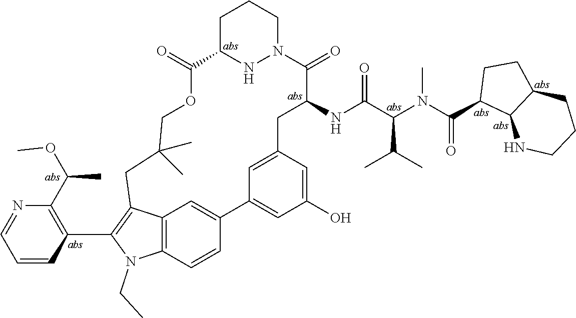 |
| AB184 | 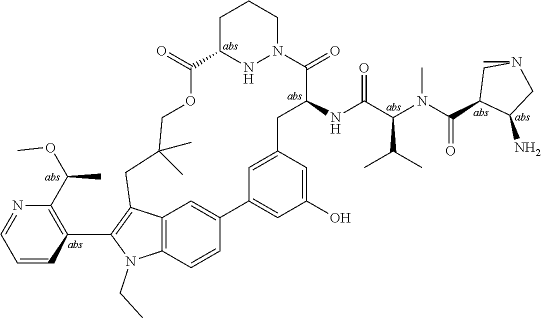 |
| AB185 | 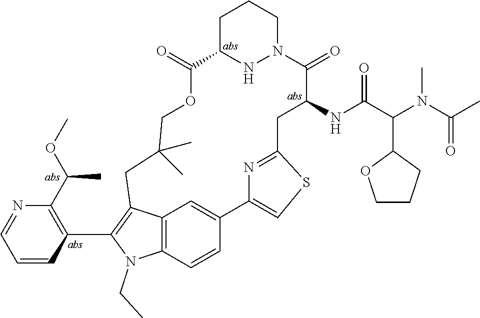 |
| AB186 | 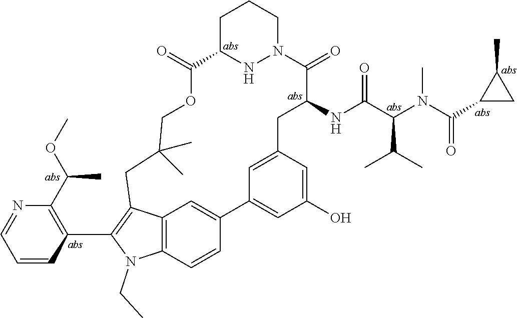 |
| AB187 | 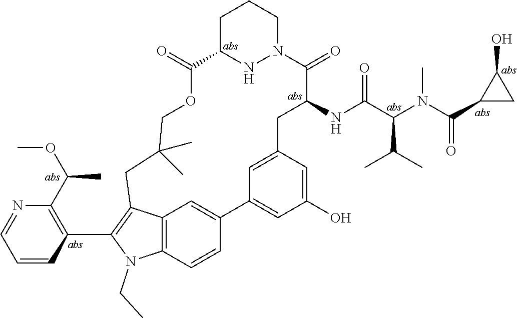 |
| AB188 | 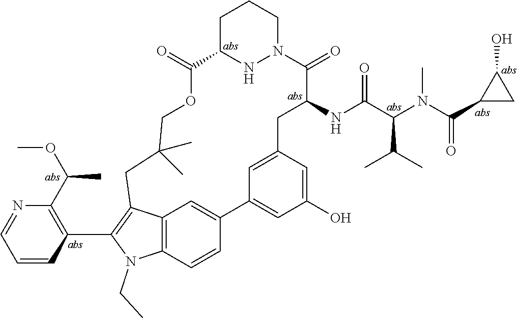 |
| AB189 | 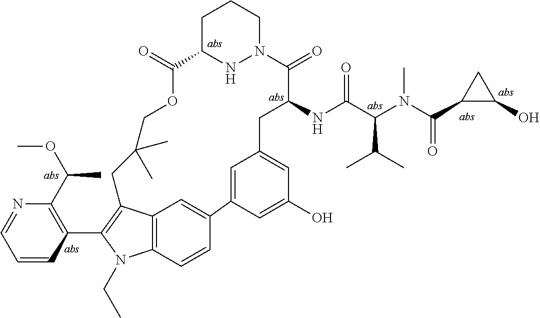 |
| AB190 | 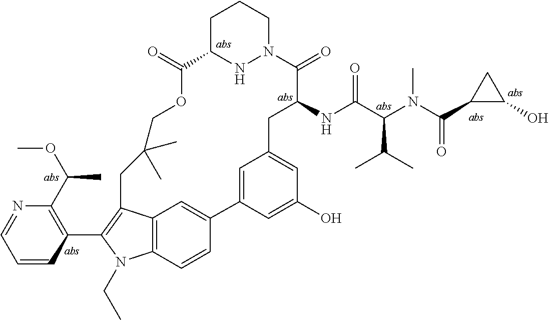 |
| AB191 | 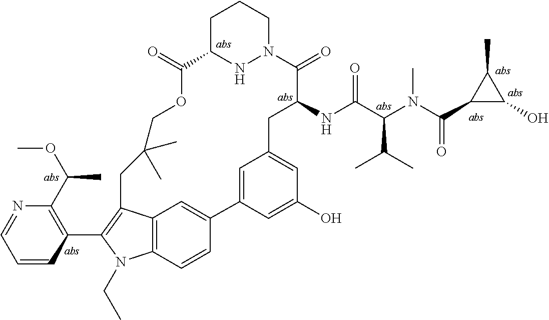 |
| AB192 | 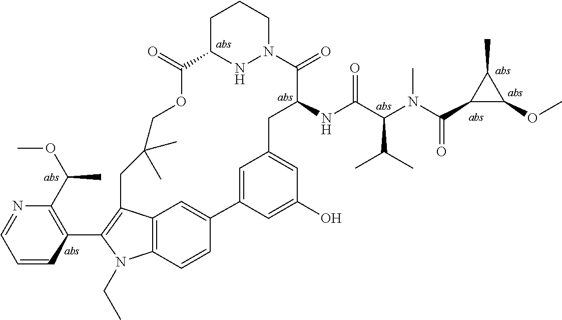 |
| AB193 | 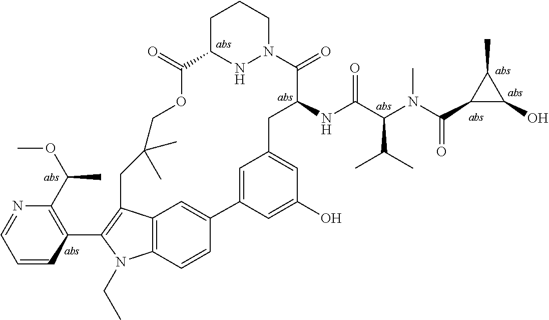 |
| AB194 | 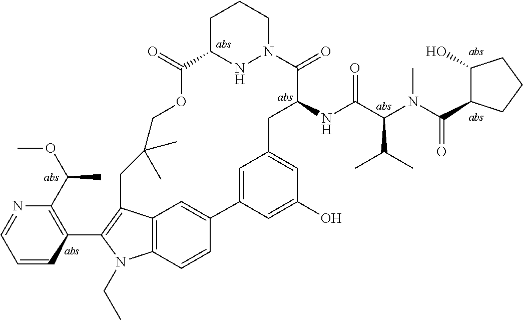 |
| AB195 | 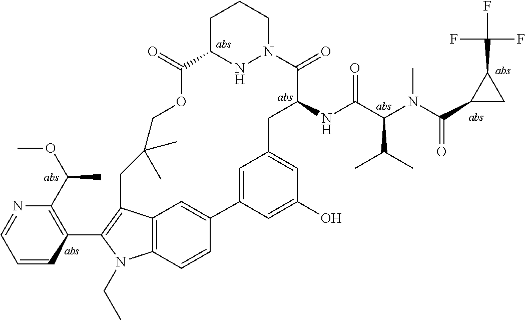 |
| AB196 | 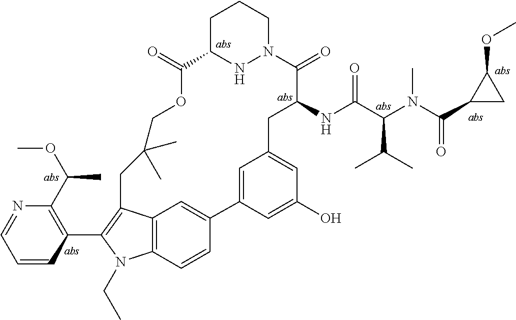 |
| AB197 | 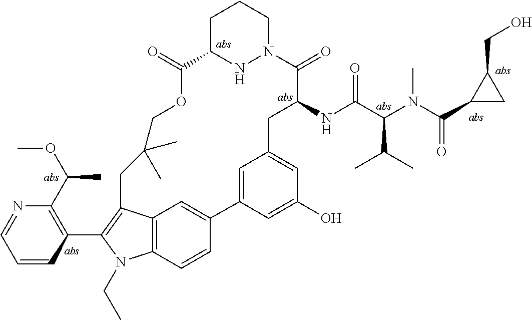 |
| AB198 | 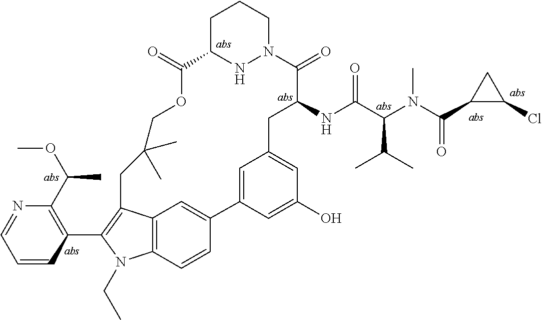 |
| AB199 | 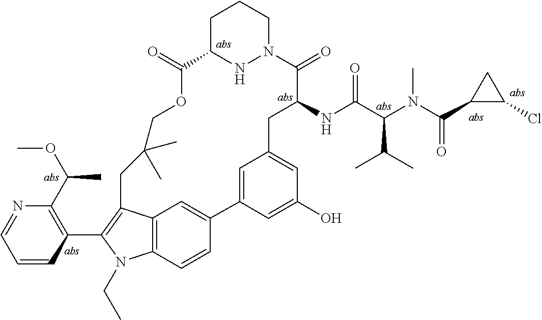 |
| AB200 | 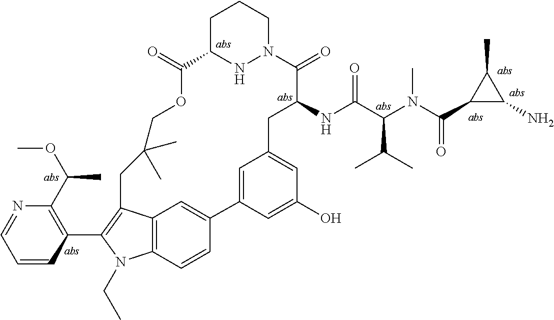 |
| AB201 | 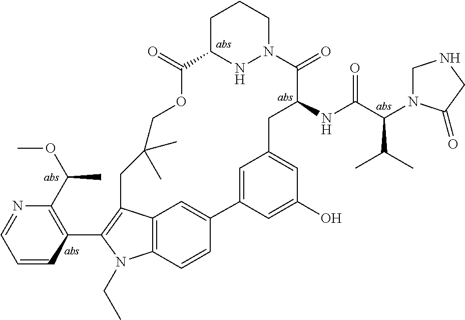 |
| AB202 | 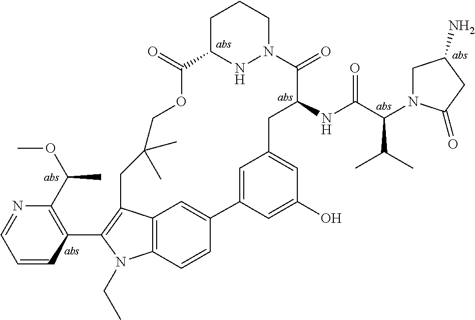 |
| AB203 | 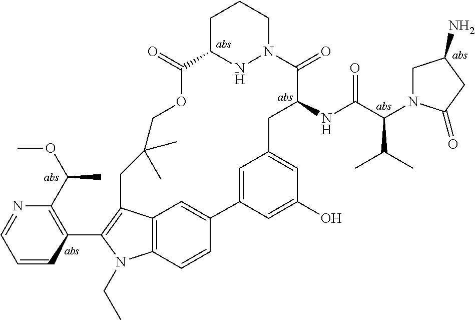 |
| AB204 | 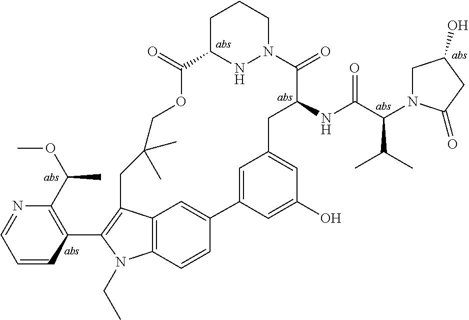 |
| AB205 | 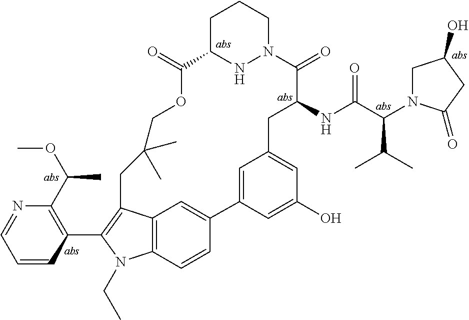 |
| AB206 | 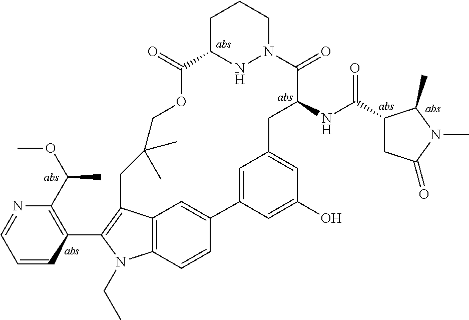 |
| AB207 | 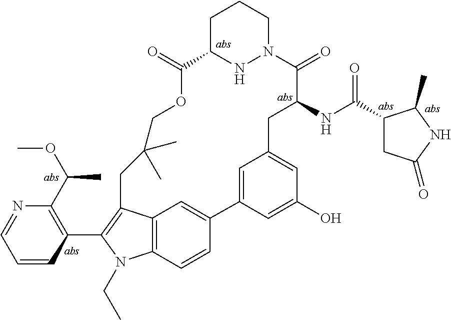 |
| AB208 | 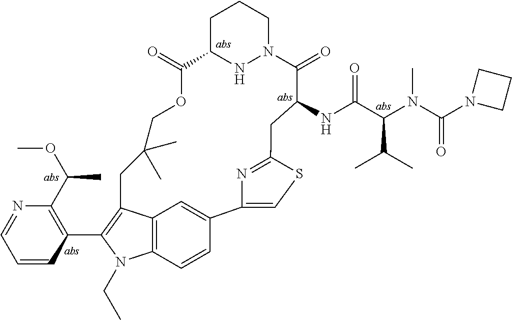 |
| AB209 | 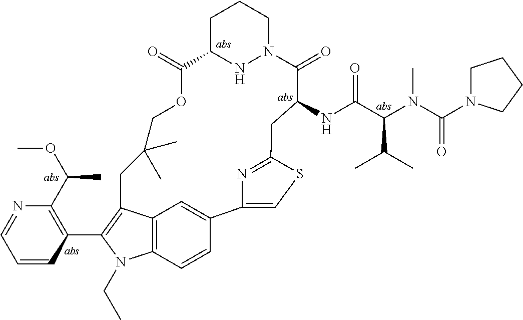 |
| AB210 | 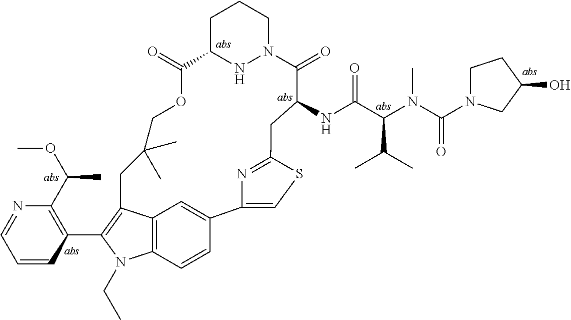 |
| AB211 | 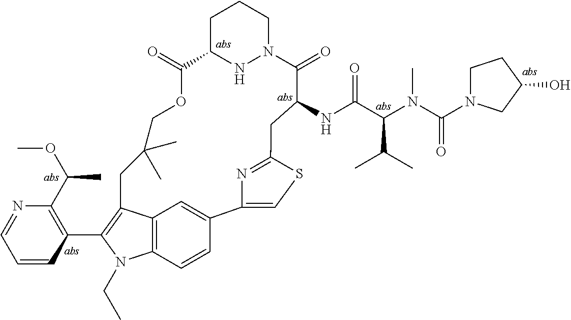 |
| AB212 | 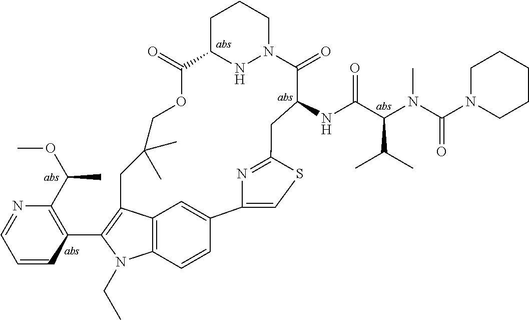 |
| AB213 | 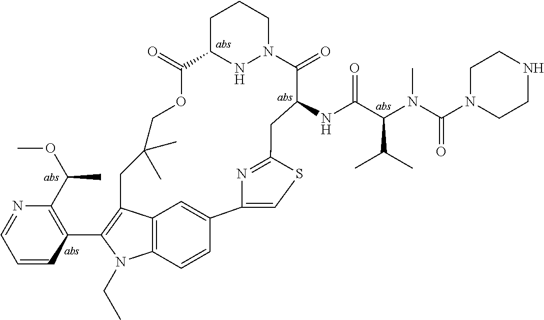 |
| AB214 | 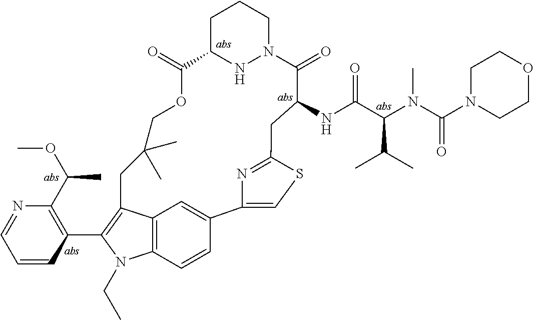 |
| AB215 | 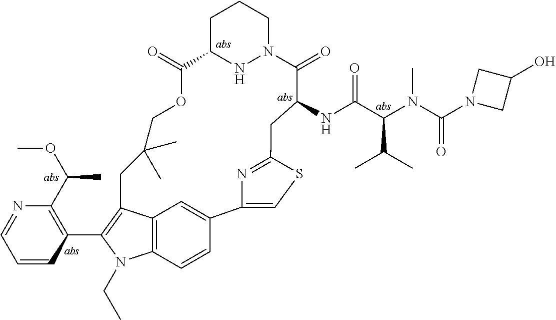 |
| AB216 | 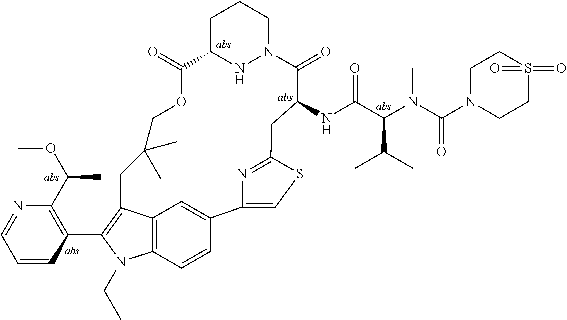 |
| AB217 | 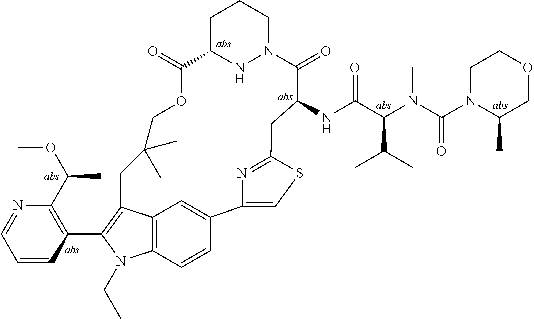 |
| AB218 | 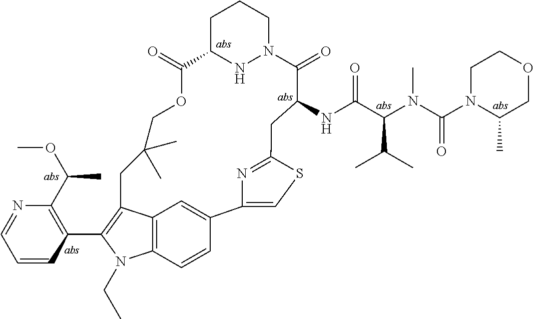 |
| AB219 | 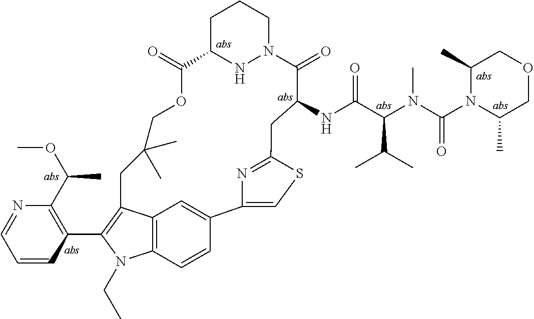 |
| AB220 | 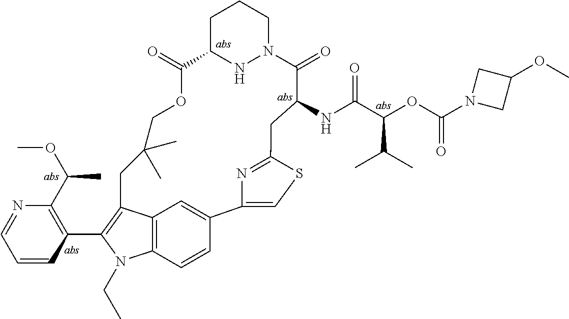 |
| AB221 | 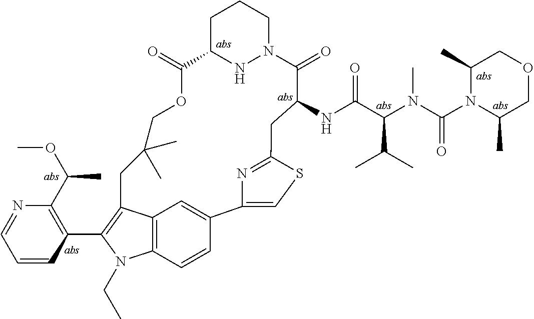 |
| AB222 | 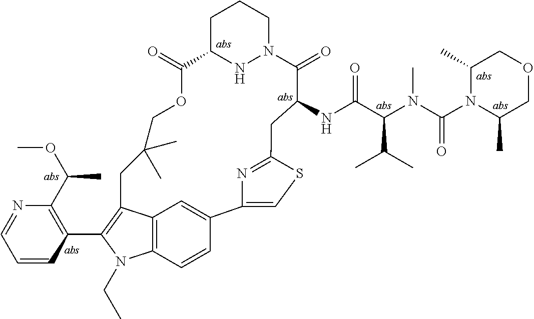 |
| AB223 | 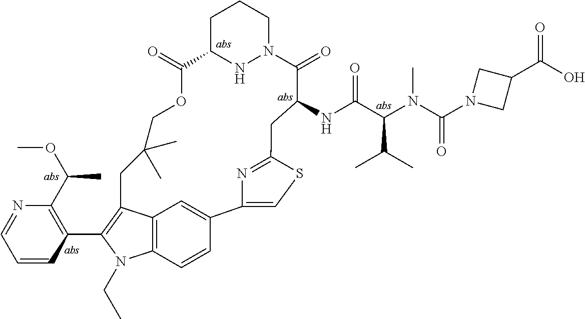 |
| AB224 | 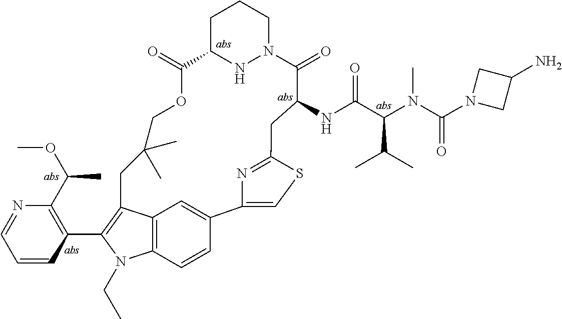 |
| AB225 | 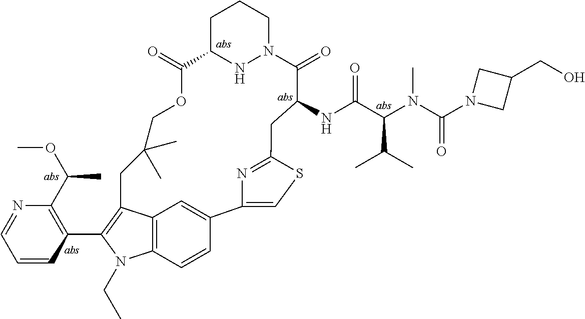 |
| AB226 | 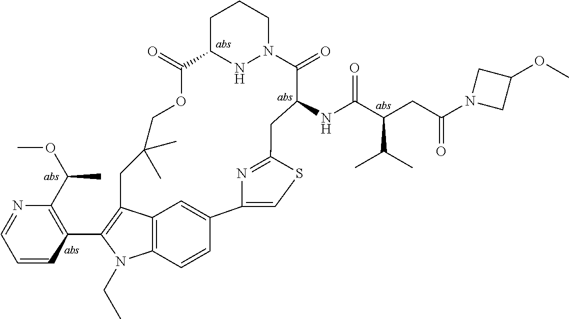 |
| AB227 | 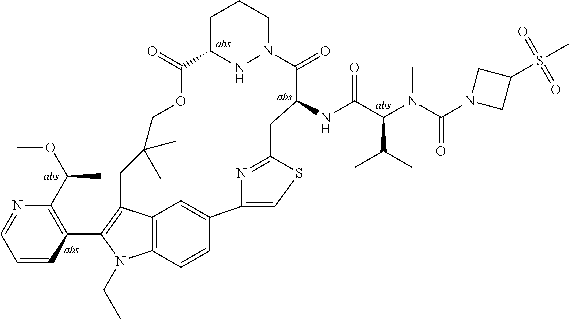 |
| AB228 | 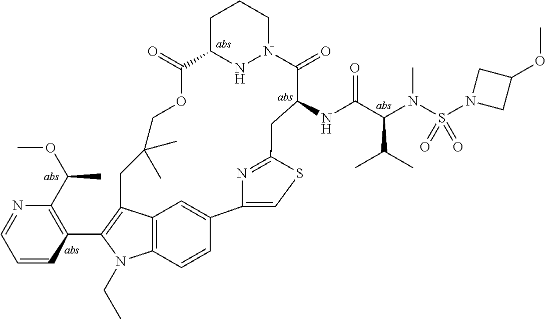 |
| AB229 | 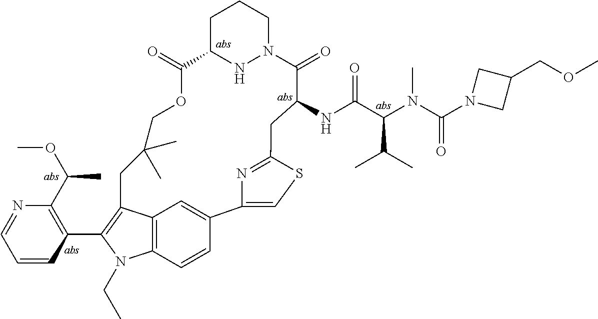 |
| AB230 | 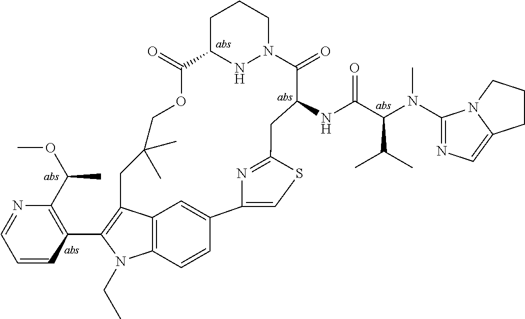 |
| AB231 | 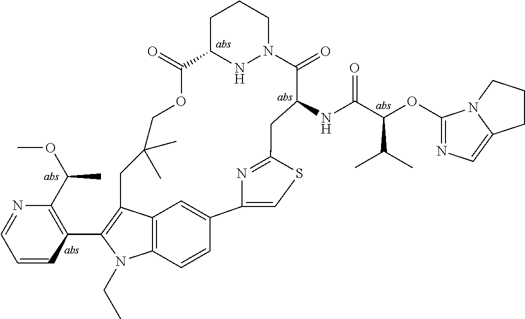 |
| AB232 | 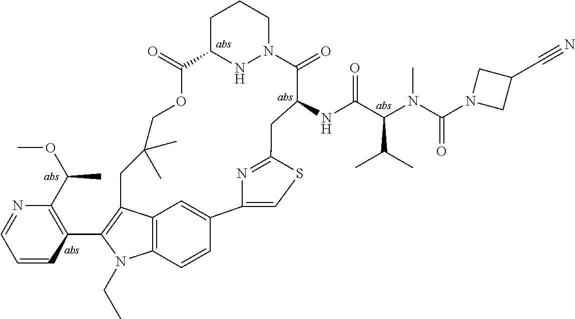 |
| AB233 | 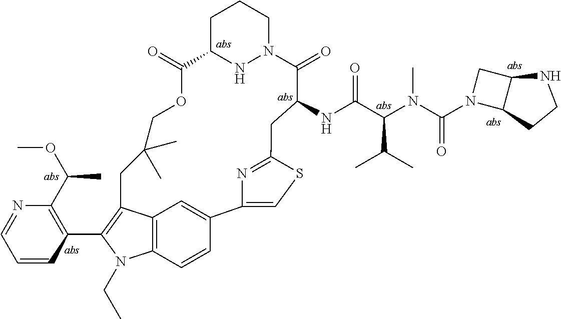 |
| AB234 | 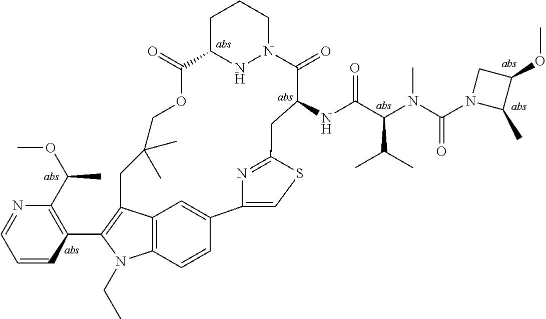 |
| AB235 | 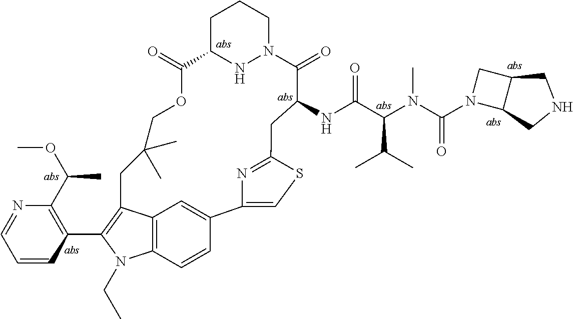 |
| AB236 | 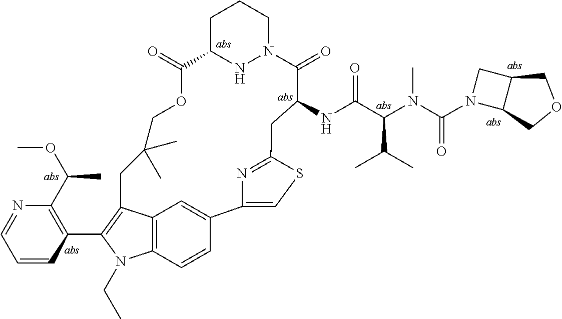 |
| AB237 | 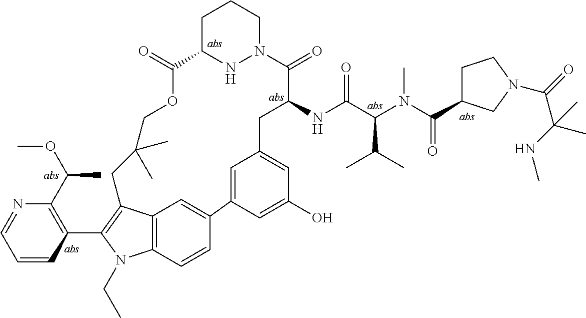 |
| AB238 | 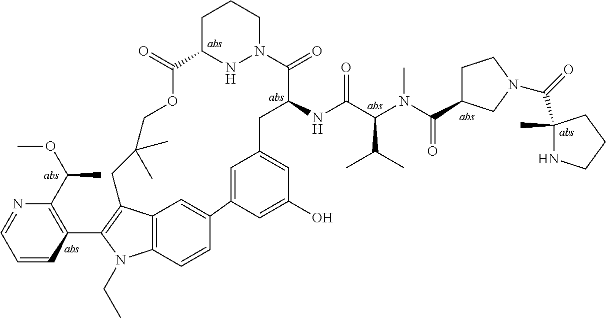 |
| AB239 | 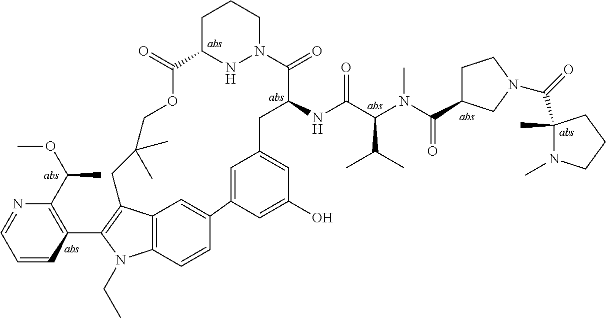 |
| AB240 | 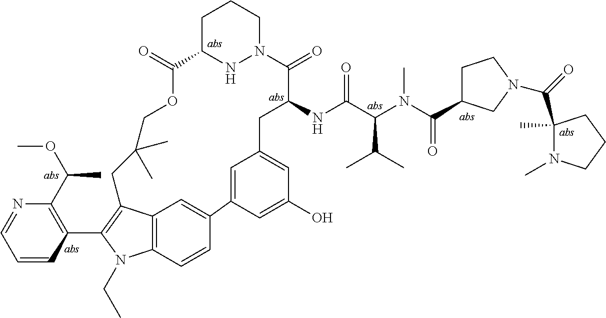 |
| AB241 | 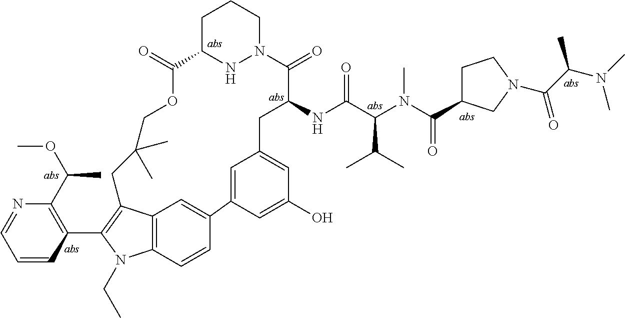 |
| AB242 | 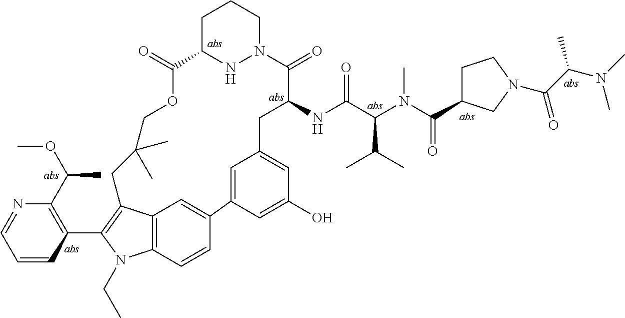 |
| AB243 | 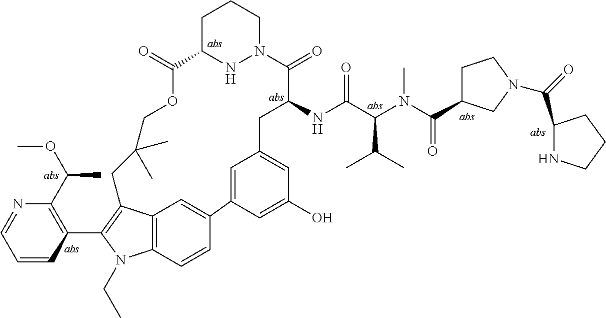 |
| AB244 | 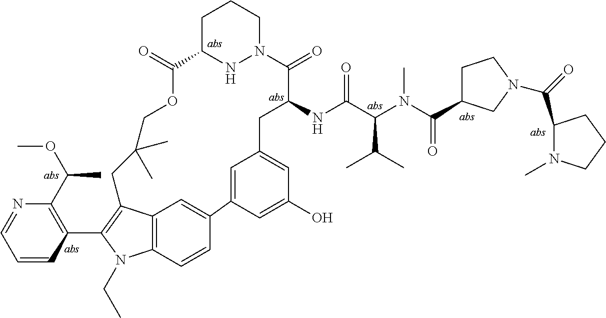 |
| AB245 | 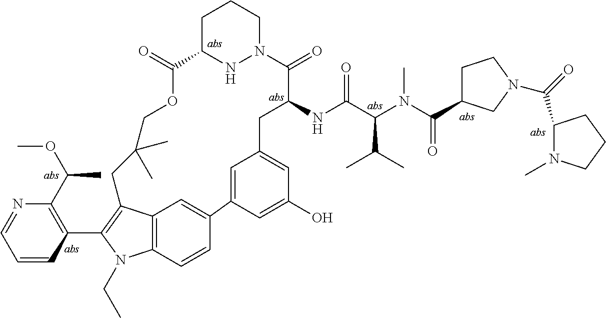 |
| AB246 | 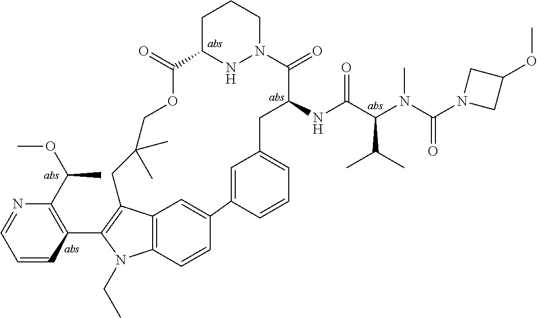 |
| AB247 | 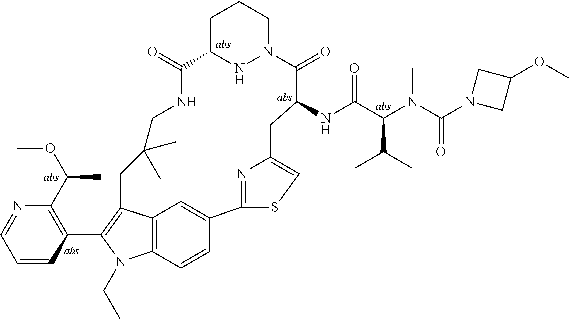 |
| AB248 | 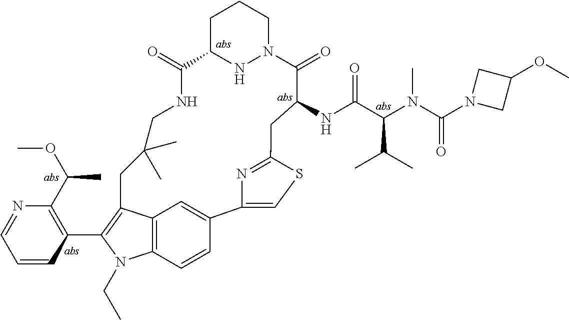 |
| AB249 | 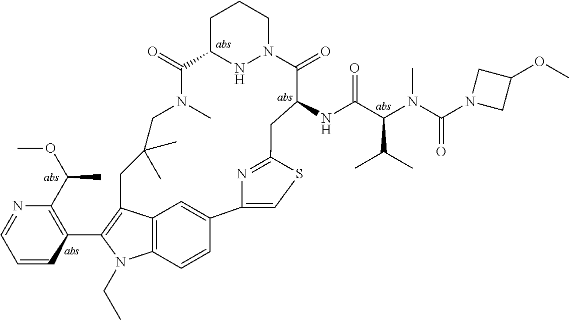 |
| AB250 | 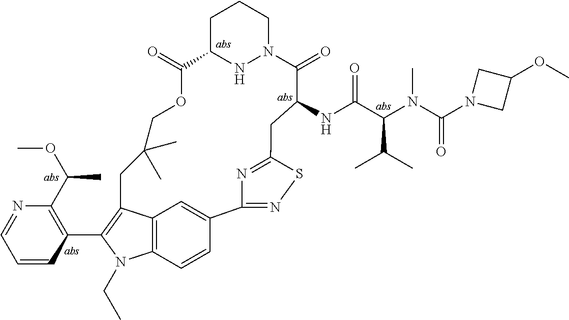 |
| AB251 | 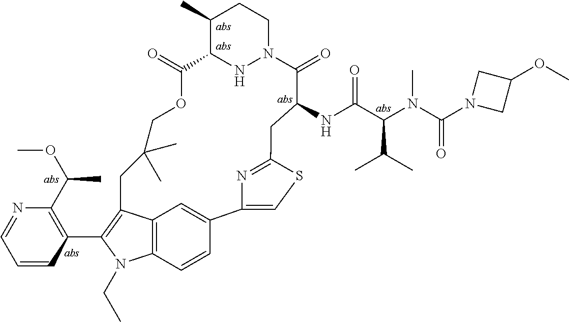 |
| AB252 | 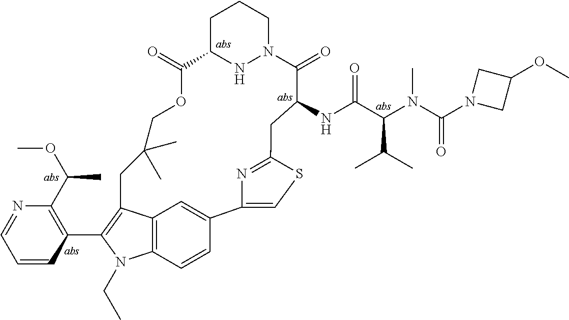 |
| AB253 | 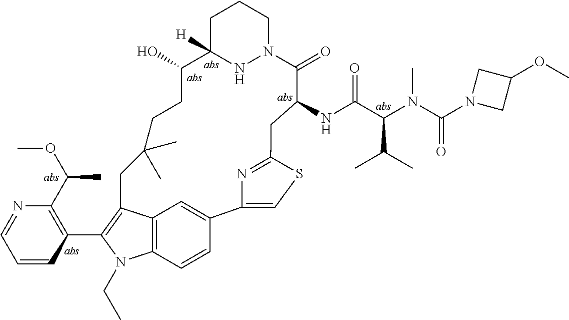 |
| AB254 | 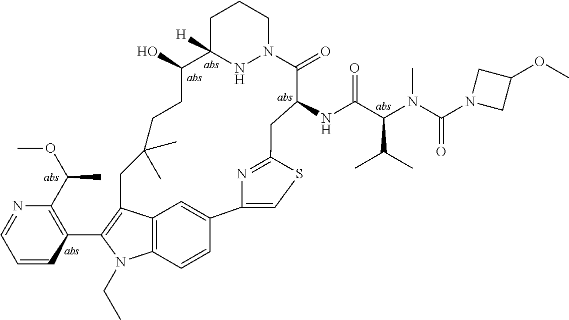 |
| AB255 | 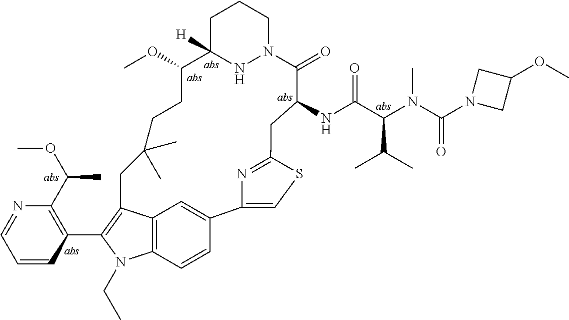 |
| AB256 | 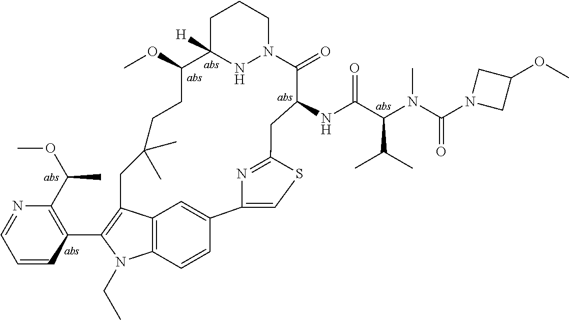 |
| AB257 | 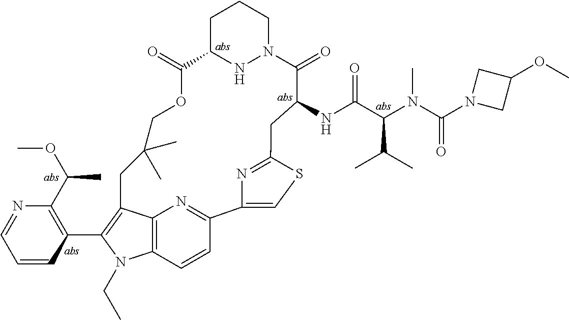 |
| AB258 | 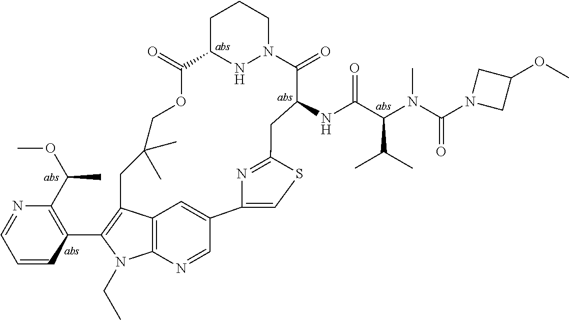 |
| AB259 | 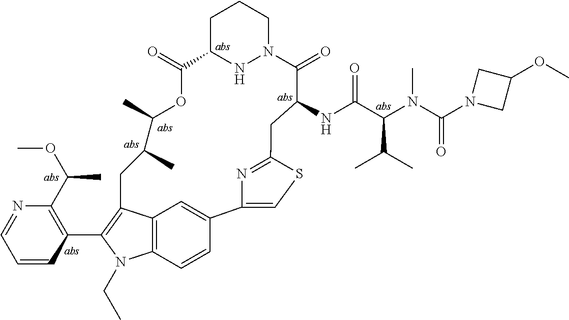 |
| AB260 | 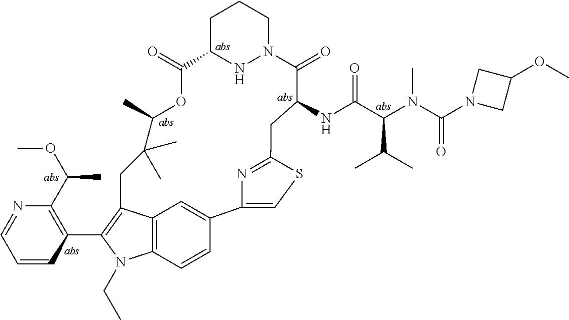 |
| AB261 | 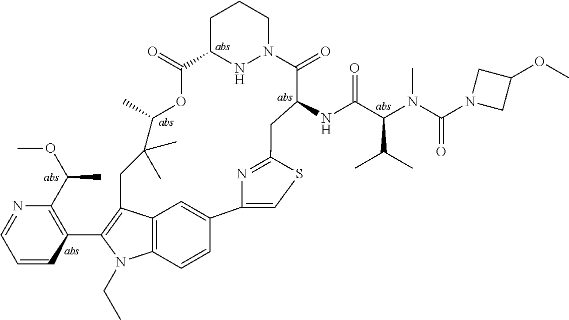 |
| AB262 | 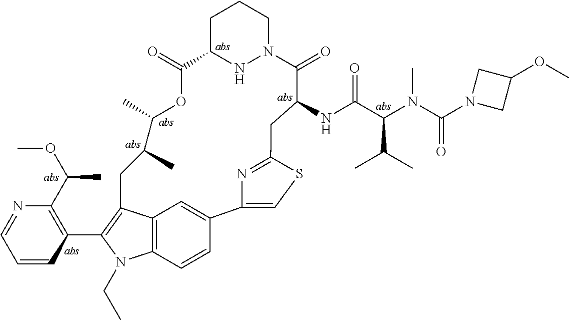 |
| AB263 | 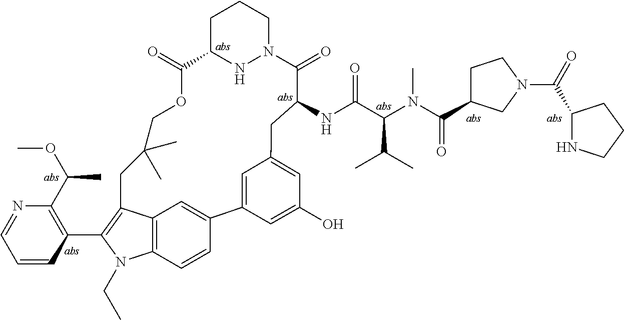 |

Note that some compoounds are shown with bonds as flat or wedged. In some instances, the relative stereochemistry has been determined; in some instances, the absolute stereochemistry has been determined. All stereoisomers of the compounds of the foregoing table are contemplated by the present invention. In particular embodiments, an atropisomer of a compound of the foregoing table is contemplated.

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below and in WO 2021/091956, together with synthetic methods known in the art of synthetic organic chemistry, or variations thereon as appreciated by those skilled in the art. These methods include but are not limited to those methods described in the Schemes below or as described in WO 2021/091956.

Compounds of Table A1 herein were prepared using methods disclosed herein or were prepared using methods disclosed herein combined with the knowledge of one of skill in the art. Compounds of Table A2 may be prepared using methods disclosed herein or may be prepared using methods disclosed herein combined with the knowledge of one of skill in the art.

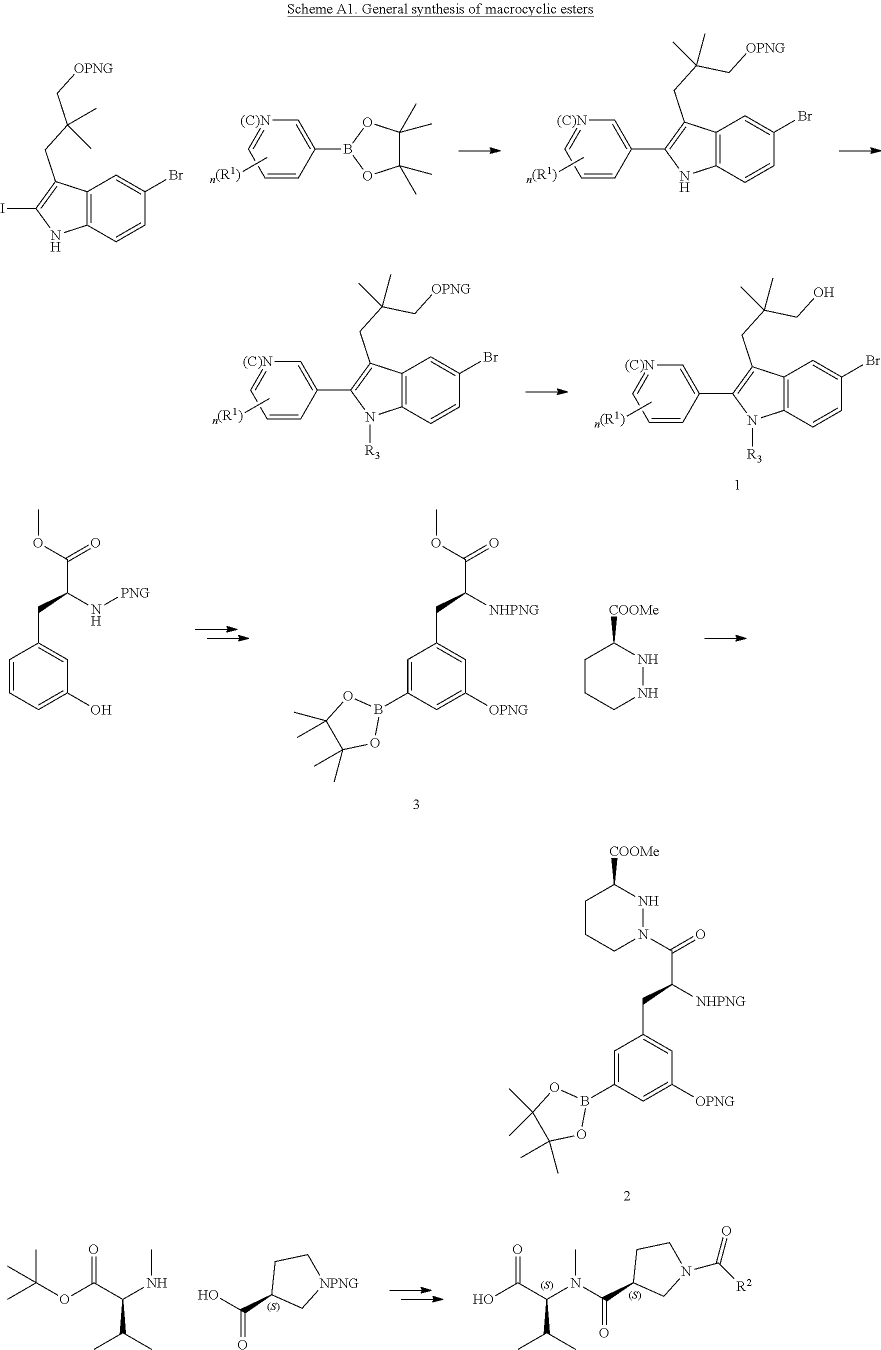

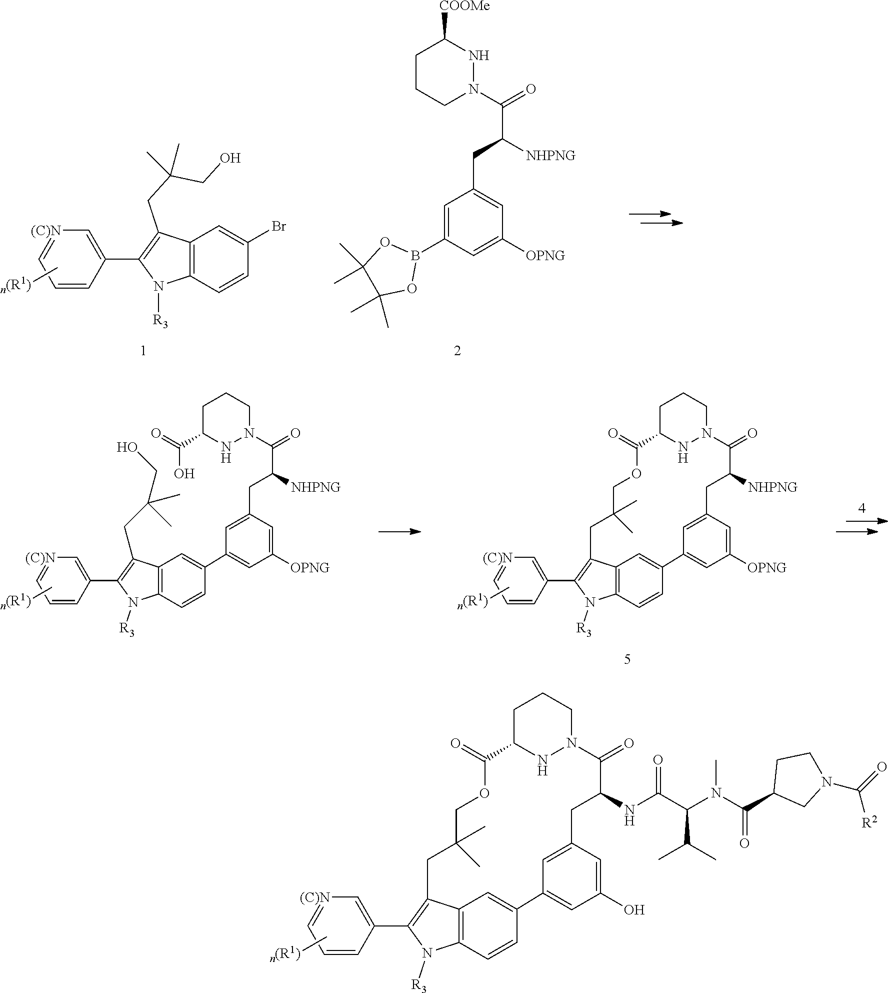

A general synthesis of macrocyclic esters is outlined in Scheme A1. An appropriately substituted Aryl Indole intermediate (1) can be prepared in three steps starting from protected 3-(5-bromo-2-iodo-1H-indol-3-yl)-2,2-dimethylpropan-1-ol and appropriately substituted boronic acid, including Palladium mediated coupling, alkylation, and de-protection reactions.

Methyl-amino-hexahydropyridazine-3-carboxylate-boronic ester (2) can be prepared in three steps, including protection, Iridium catalyst mediated borylation, and coupling with methyl (S)-hexahydropyridazine-3-carboxylate.

An appropriately substituted acetylpyrrolidine-3-carbonyl-N-methyl-L-valine (4) can be made by coupling of methyl-L-valinate and protected (S)-pyrrolidine-3-carboxylic acid, followed by deprotection, coupling with an appropriately substituted carboxylic acid, and a hydrolysis step.

The final macrocyclic esters can be made by coupling of methyl-amino-hexahydropyridazine-3-carboxylate-boronic ester (2) and intermediate (1) in the presence of Pd catalyst followed by hydrolysis and macrolactonization steps to result in an appropriately protected macrocyclic intermediate (5). Deprotection and coupling with an appropriately substituted acetylpyrrolidine-3-carbonyl-N-methyl-L-valine (4) results in a macrocyclic product. Additional deprotection or functionalization steps are be required to produce a final compound. For example, a person of skill in the art would be able to install into a macrocyclic ester a desired —B-L-W group of a compound of Formula (A1), where B, L and W are defined herein, including by using methods exemplified in the Example section herein.

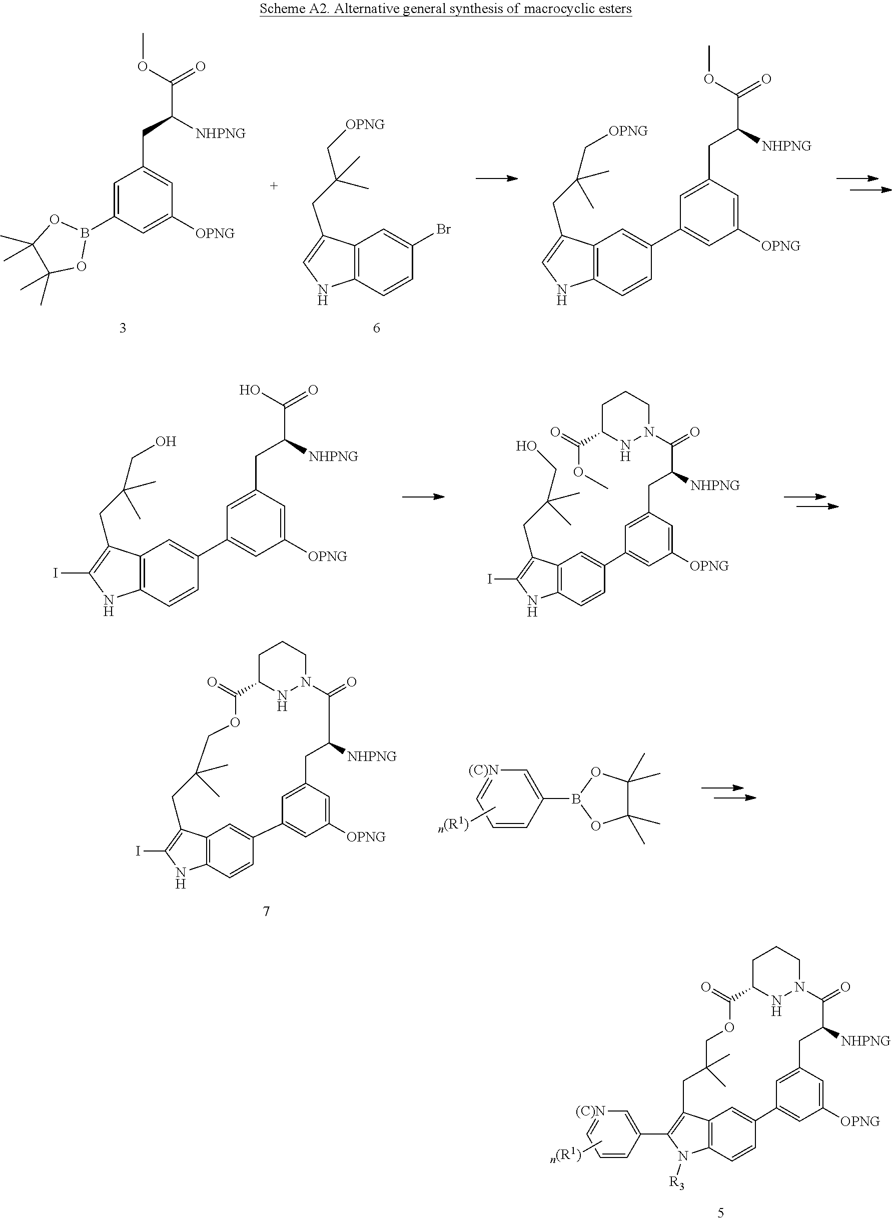

Alternatively, macrocyclic esters can be prepared as described in Scheme 2. An appropriately protected bromo-indolyl (6) can be coupled in the presence of Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of Pd catalyst with an appropriately substituted boronic ester and alkylation can yield fully a protected macrocycle (5). Additional deprotection or functionalization steps are required to produce a final compound. For example, a person of skill in the art would be able to install into a macrocyclic ester a desired —B-L-W group of a compound of Formula (A1), where B, L and W are defined herein, including by using methods exemplified in the Example section herein.

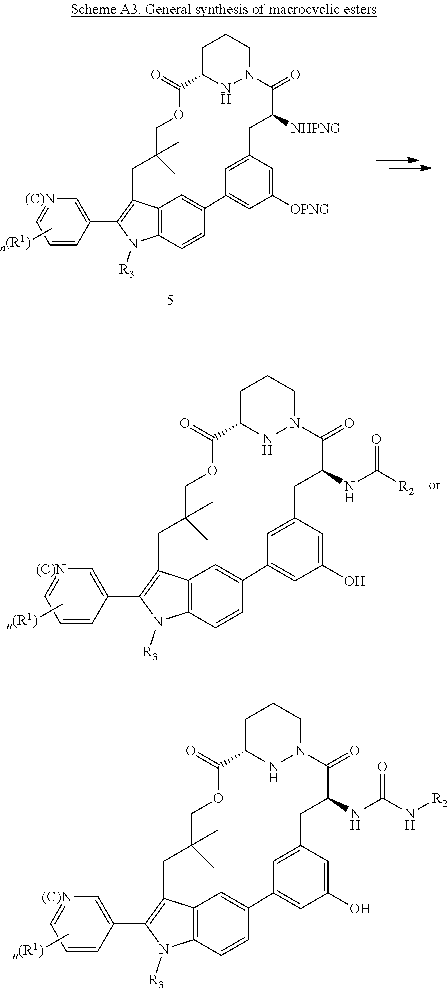

Alternatively, fully a protected macrocycle (5) can be deprotected and coupled with an appropriately substitututed coupling partners, and deprotected to results in a macrocyclic product. Additional deprotection or functionalization steps are be required to produce a final compound. For example, a person of skill in the art would be able to install into a macrocyclic ester a desired —B-L-W group of a compound of Formula (A1), where B, L and W are defined herein, including by using methods exemplified in the Example section herein.

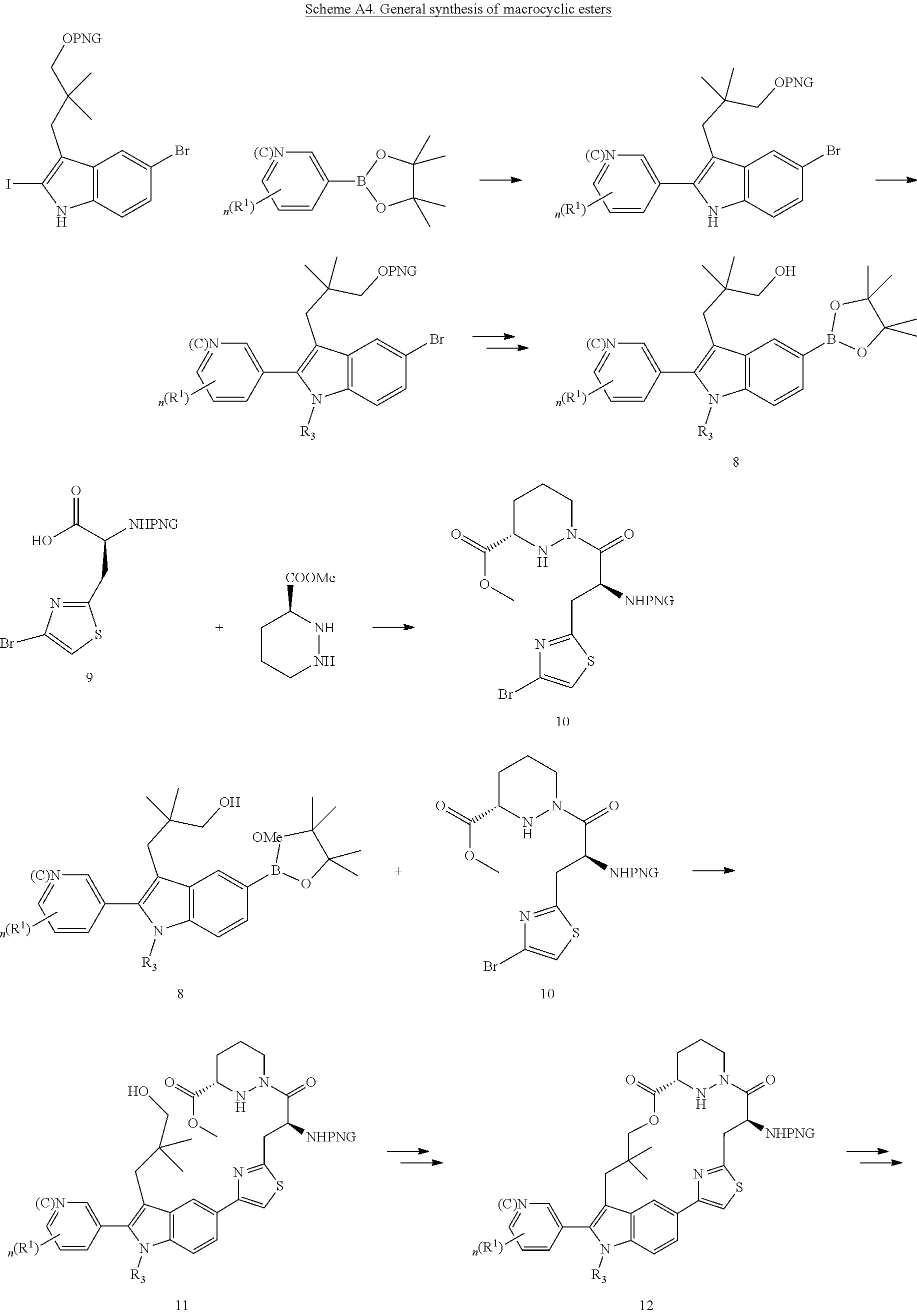

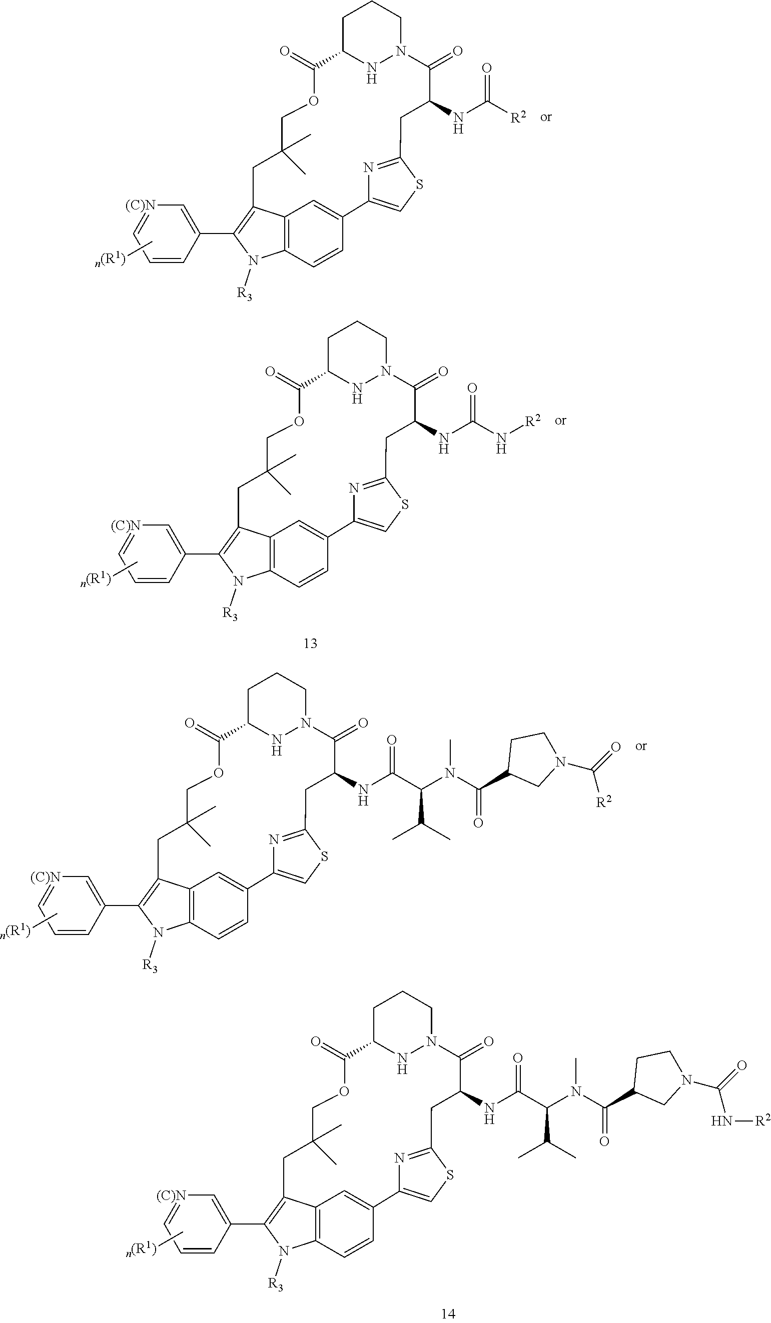

An alternative general synthesis of macrocyclic esters is outlined in Scheme A4. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted carboxylic acid (or other coupling partner) or intermediate 4 can result in a macrocyclic product. Additional deprotection or functionalization steps could be required to produce a final compound 13 or 14.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the WO 2021/091956. For example, a person of skill in the art would be able to install into a macrocyclic ester a desired —B-L-W group of a compound of Formula (AI), where B, L and W are defined herein, including by using methods exemplified in the Example section herein.

In some embodiments, the RAS(ON) inhibitor is a compound, or a pharmaceutically acceptable salt thereof, having the structure of Formula BI:

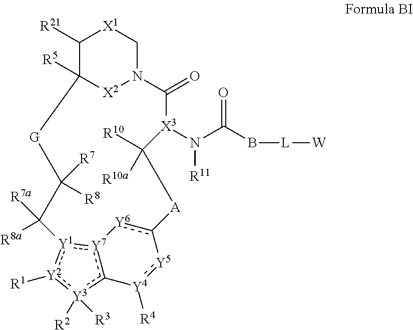

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments of Formula BI, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI, R²¹ is hydrogen.

In some embodiments, provided herein is a compound, or pharmaceutically acceptable salt thereof, having the structure of Formula BIa:

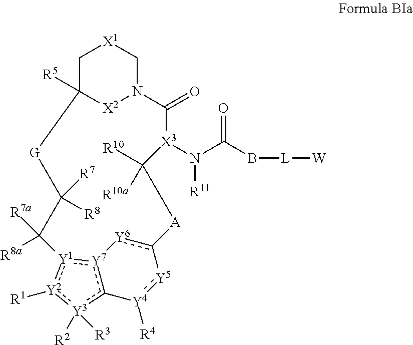

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the disclosure features a compound, or pharmaceutically acceptable salt thereof, of structural Formula BIb:

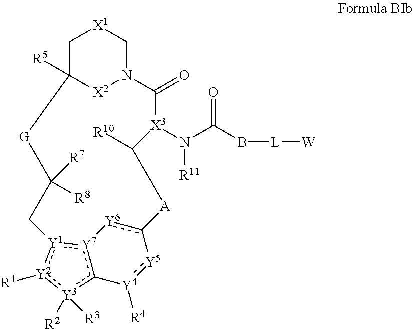

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(0-C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula BI and subformula thereof, G is optionally substituted C₁-C₄ heteroalkylene.

In some embodiments, a compound having the structure of Formula BIc is provided, or a pharmaceutically acceptable salt thereof:

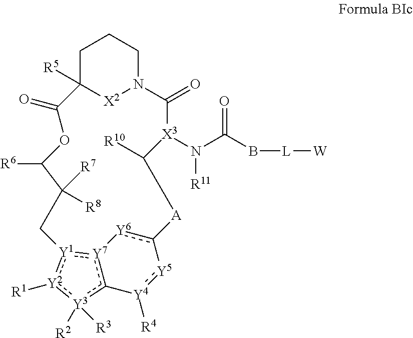

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula BI and subformula thereof, X² is NH. In some embodiments of Formula BI and subformula thereof, X³ is CH. In some embodiments of Formula BI and subformula thereof, R¹¹ is hydrogen. In some embodiments of Formula BI and subformula thereof, R¹¹ is C₁-C₃ alkyl. In some embodiments of Formula BI and subformula thereof, R¹¹ is methyl.

In some embodiments, the RAS(ON) inhibitor has the structure of Formula Bid, or a pharmaceutically acceptable salt thereof:

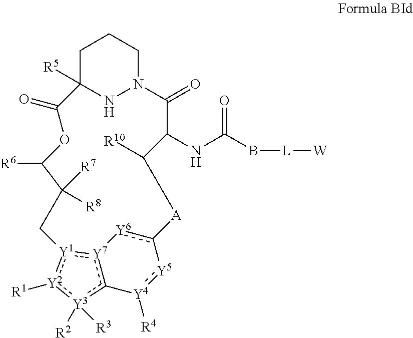

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula BI and subformula thereof, X¹ is optionally substituted C₁-C₂ alkylene. In some embodiments, X¹ is methylene. In some embodiments of Formula BI and subformula thereof, X¹ is methylene substituted with a C₁-C₆ alkyl group or a halogen. In some embodiments, X¹ is —CH(Br)—. In some embodiments, X¹ is —CH(CH₃)—. In some embodiments of Formula BI and subformula thereof, R⁵ is hydrogen. In some embodiments of Formula BI and subformula thereof, R⁵ is C₁-C₄ alkyl optionally substituted with halogen. In some embodiments, R⁵ is methyl. In some embodiments of Formula BI and subformula thereof, Y⁴ is C. In some embodiments of Formula BI and subformula thereof, R⁴ is hydrogen. In some embodiments of Formula BI and subformula thereof, Y⁵ is CH. In some embodiments of Formula BI and subformula thereof, Y⁶ is CH. In some embodiments of Formula BI and subformula thereof, Y¹ is C. In some embodiments of Formula BI and subformula thereof, Y² is C. In some embodiments of Formula BI and subformula thereof, Y³ is N. In some embodiments of Formula BI and subformula thereof, R³ is absent. In some embodiments of Formula BI and subformula thereof, Y⁷ is C.

In some embodiments, the RAS(ON) inhibitor has the structure of Formula BIe, or a pharmaceutically acceptable salt thereof:

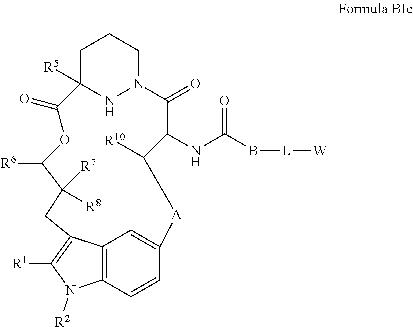

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula BI and subformula thereof, R⁶ is hydrogen. In some embodiments, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl. In some embodiments, R² is fluoroalkyl. In some embodiments, R² is ethyl. In some embodiments, R² is —CH₂CF₃. In some embodiments, R² is C₂-C₆ alkynyl. In some embodiments, R² is —CHC═CH. In some embodiments, R² is —CH₂C═CCH₃. In some embodiments, R⁷ is optionally substituted C₁-C₃ alkyl. In some embodiments, R⁷ is C₁-C₃ alkyl. In some embodiments, R⁸ is optionally substituted C₁-C₃ alkyl. In some embodiments, R⁸ is C₁-C₃ alkyl.

In some embodiments, the RAS(ON) inhibitor has the structure of Formula BIf, or a pharmaceutically acceptable salt thereof:

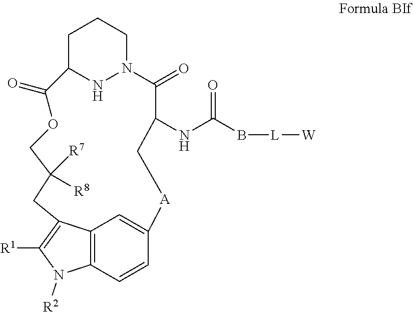

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI and subformula thereof, R¹ is optionally substituted 6 to 10-membered aryl, optionally substituted 3 to 6-membered cycloalkenyl, or optionally substituted 5 to 10-membered heteroaryl. In some embodiments, R¹ is optionally substituted 6-membered aryl, optionally substituted 6-membered cycloalkenyl, or optionally substituted 6-membered heteroaryl.

In some embodiments of Formula BI and subformula thereof, R₁ is

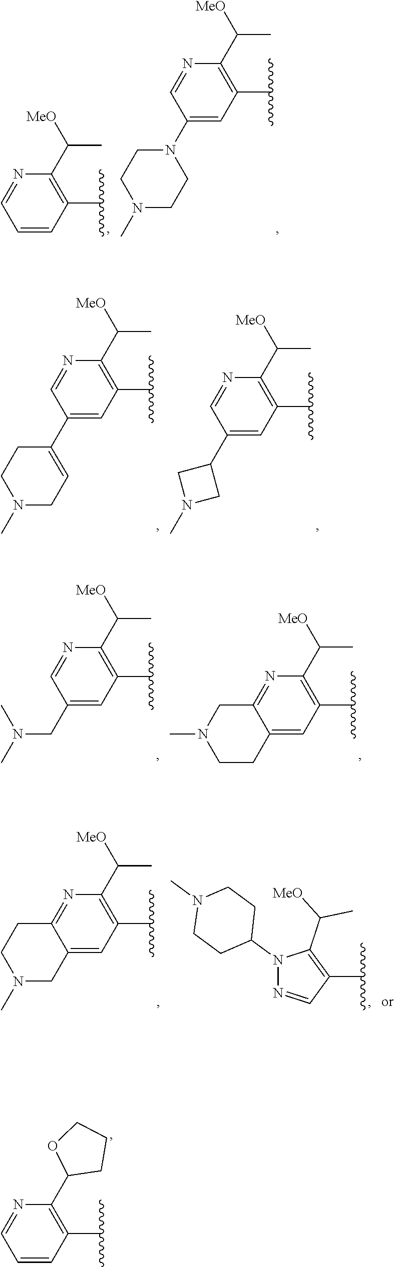

or a stereoisomer (e.g., atropisomer) thereof. In some embodiments of Formula BI and subformula thereof, R₁

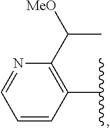 [SMILES: *c1cccnc1C(C)OC]

or a stereoisomer (e.g., atropisomer) thereof. In some embodiments of Formula BI and subformula thereof, R₁ is

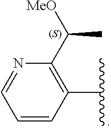

In some embodiments, the RAS(ON) inhibitor has the structure of Formula Big, or a pharmaceutically acceptable salt thereof:

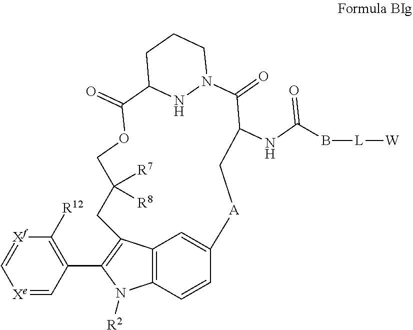

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl
X^{e} and X^{f} are, independently, N or CH; and
R¹² is optionally substituted C₁-C₆ alkyl, optionally substituted C₁-C₆ heteroalkyl, or optionally substituted 3 to 6-membered heterocycloalkylene.

In some embodiments of Formula BI and subformula thereof, X^{e} is N and X^{f} is CH. In some embodiments, X^{e} is CH and X^{f} is N.

In some embodiments of Formula BI and subformula thereof, R¹² is optionally substituted C₁-C₆ heteroalkyl. In some embodiments, R¹² is

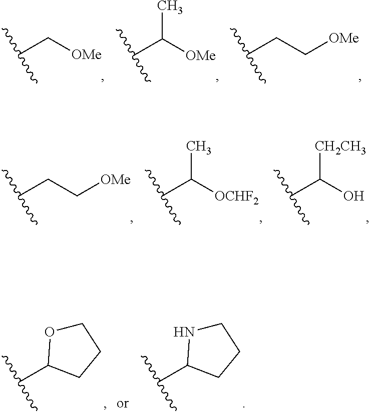

In some embodiments, R¹² is

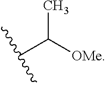 [SMILES: *C(C)OC]

In some embodiments, the RAS(ON) inhibitor has the structure of Formula BVI, or a pharmaceutically acceptable salt thereof:

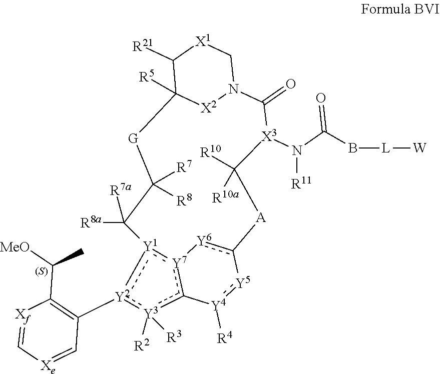

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R²¹ is hydrogen or C₁-C₃ alkyl (e.g., methyl); and
X^{e} and X^{f} are, independently, N or CH.

In some embodiments, the RAS(ON) inhibitor has the structure of Formula BVIa, or a pharmaceutically acceptable salt thereof:

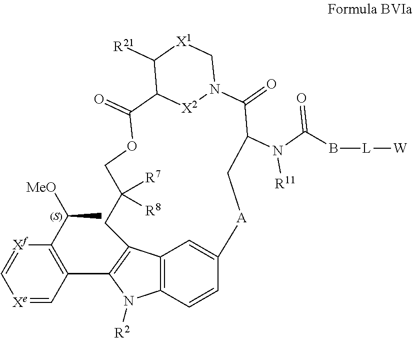

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of Formula BI and subformula thereof, X^{e} is N and X^{f} is CH. In some embodiments, X^{e} is CH and X^{f} is N.

In some embodiments, the RAS(ON) inhibitor has the structure of Formula BVIb, or a pharmaceutically acceptable salt thereof:

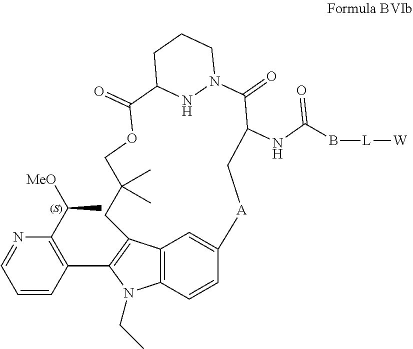

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
L is absent or a linker; and
W is a cross-linking group comprising a vinyl ketone, a vinyl sulfone, an ynone, or an alkynyl sulfone.

In some embodiments of formula BI or subformula thereof, A is optionally substituted 6-membered arylene.

In some embodiments, the RAS(ON) inhibitor has the structure of Formula BVIc, or a pharmaceutically acceptable salt thereof:

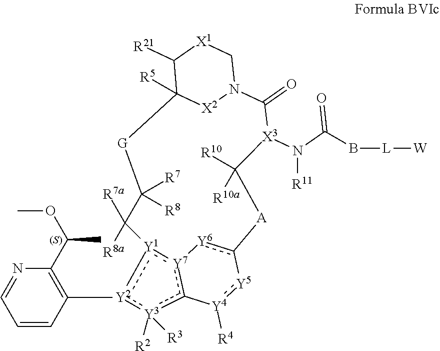

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments of Formula BI and subformula thereof, A has the structure:

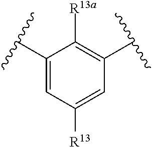

wherein R¹³ is hydrogen, halo, hydroxy, amino, optionally substituted C₁-C₆ alkyl, or optionally substituted C₁-C₆ heteroalkyl; and R^{13a} is hydrogen or halo. In some embodiments, R¹³ is hydrogen. In some embodiments, R¹³ and R^{13a} are each hydrogen. In some embodiments, R¹³ is hydroxy, methyl, fluoro, or difluoromethyl.

In some embodiments of Formula BI and subformula thereof, A is optionally substituted 5 to 6-membered heteroarylene. In some embodiments, A is:

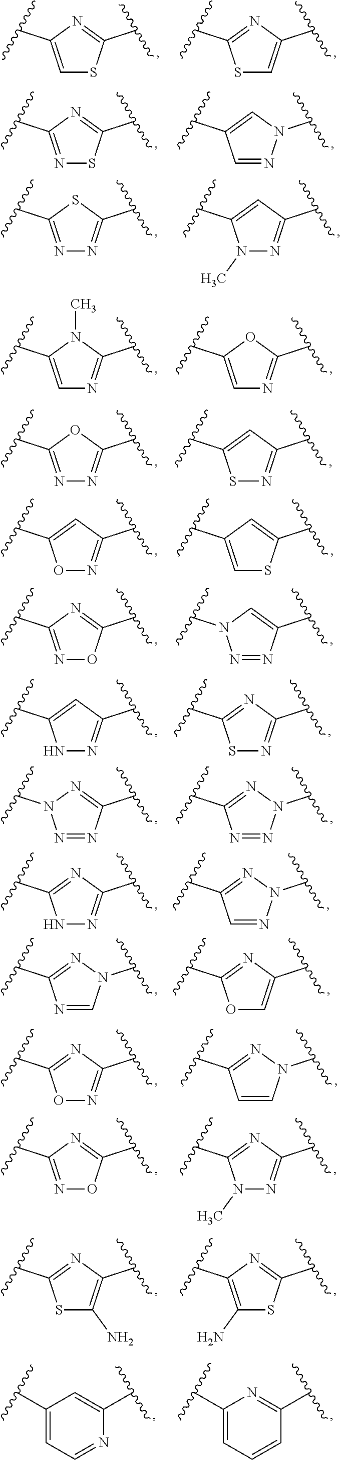

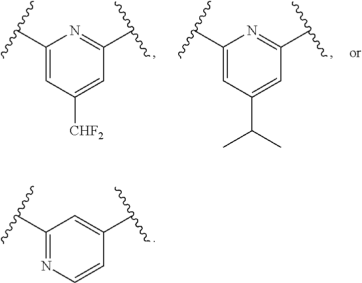 [SMILES: *c1cc(C(F)F)cc(*)n1 ; *c1cc(C(C)C)cc(*)n1 ; *c1ccnc(*)c1]

In some embodiments of Formula BI and subformula thereof, A is optionally substituted C₁-C₄ heteroalkylene. In some embodiments, A is:

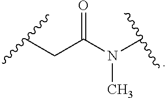 [SMILES: *CC(=O)N(*)C]

In some embodiments of Formula BI and subformula thereof, A is optionally substituted 3 to 6-membered heterocycloalkylene. In some embodiments, A is:

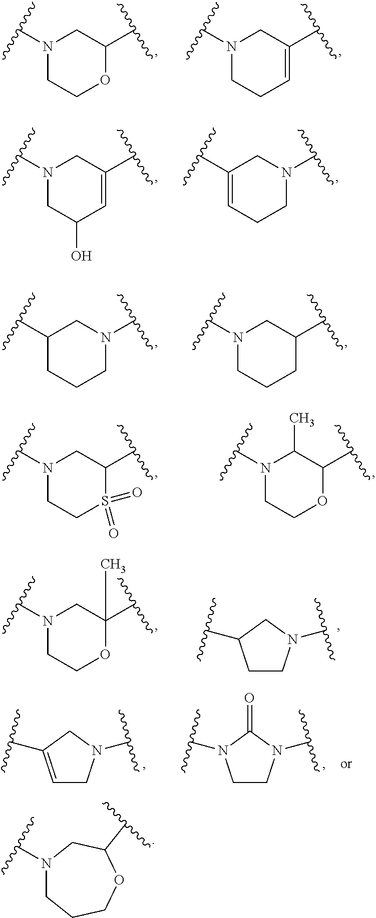

In some embodiments, A is

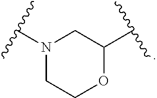 [SMILES: *C1CN(*)CCO1]

In some embodiments of Formula BI and subformula thereof, B is —CHR⁹—. In some embodiments of Formula BI and subformula thereof, R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl. In some embodiments, R⁹ is:

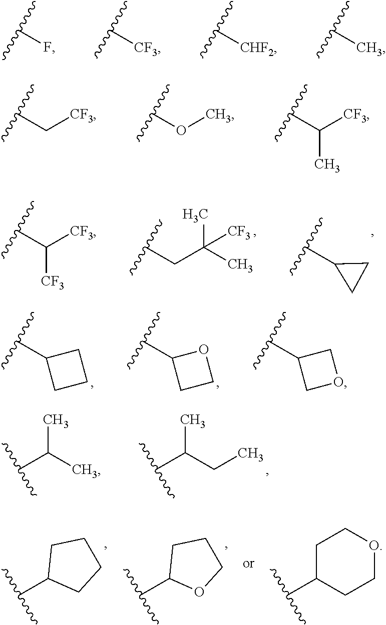

In some embodiments, R⁹ is:

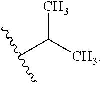 [SMILES: *C(C)C]

In some embodiments, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI and subformula thereof, B is optionally substituted 6-membered arylene. In some embodiments, B is 6-membered arylene. In some embodiments, B is:

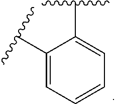 [SMILES: *c1ccccc1*]

In some embodiments of Formula BI and subformula thereof, R⁷ is methyl.

In some embodiments of Formula BI and subformula thereof, R⁸ is methyl.

In some embodiments of Formula BI and subformula thereof, R²¹ is hydrogen.

In some embodiments of Formula BI and subformula thereof, the linker is the structure of Formula BII:

A¹-(B¹)_{f}—(C¹)_{g}—(B²)ₕ-(D¹)-(B³)ᵢ—(C²)ⱼ—(B⁴)ₖ-A² Formula BII

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁₋₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments, the linker is acyclic. In some embodiments, linker has the structure of Formula BIIa:

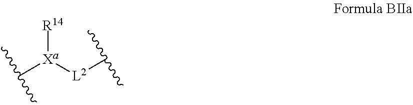

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

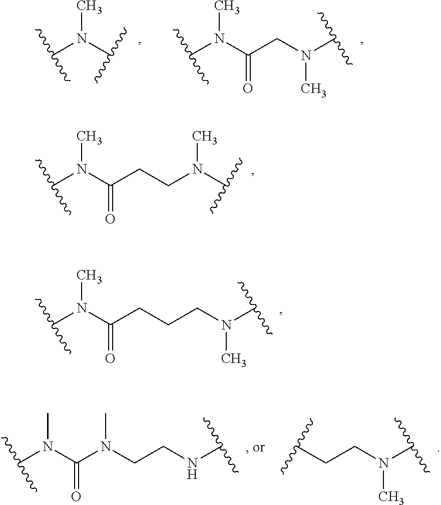

In some embodiments of Formula BI and subformula thereof, the linker is or comprises a cyclic moiety. In some embodiments, the linker has the structure of Formula BIIb:

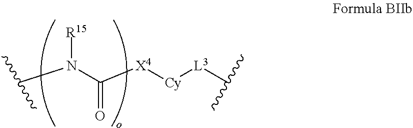

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
X⁴ is absent, optionally substituted C₁-C₄ alkylene, O, NCH₃, or optionally substituted C₁-C₄ heteroalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments of Formula BI and subformula thereof, the linker has the structure of Formula BIIb-1:

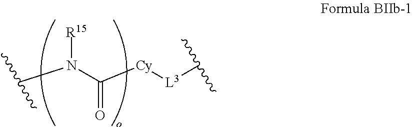

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments of Formula BI and subformula thereof, the linker has the structure of Formula BIIc:

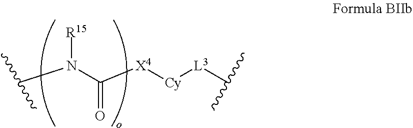

wherein R¹⁵ is hydrogen, optionally substituted C₁-C₆ alkyl, optionally substituted 3 to 8-membered cycloalkylene, or optionally substituted 3 to 8-membered heterocycloalkylene; and
R^{15a}, R^{15b}, R^{15c}, R^{15d}, R^{15e}, R^{15f}, and R^{15g} are, independently, hydrogen, halo, hydroxy, cyano, amino, optionally substituted C₁-C₆ alkyl, optionally substituted C₁-C₆ alkoxy, or, or R^{15b} and R^{15d} combine with the carbons to which they are attached to form an optionally substituted 3 to 8-membered cycloalkylene, or optionally substituted 3 to 8-membered heterocycloalkylene.

In some embodiments of Formula BI and subformula thereof, the linker has the structure:

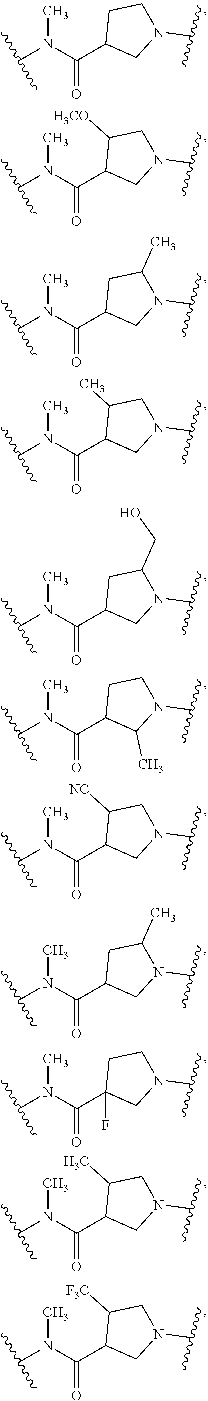

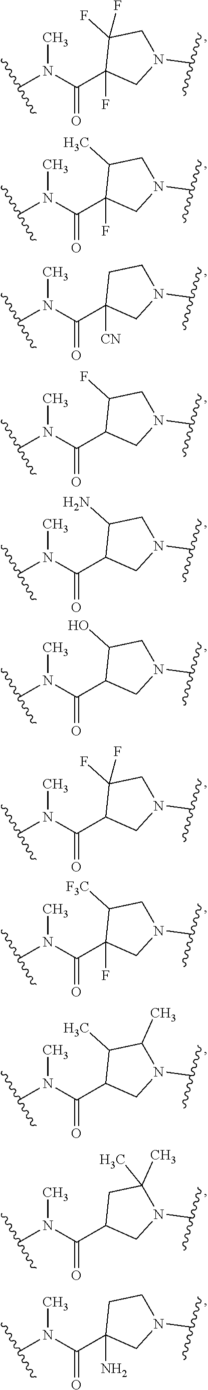

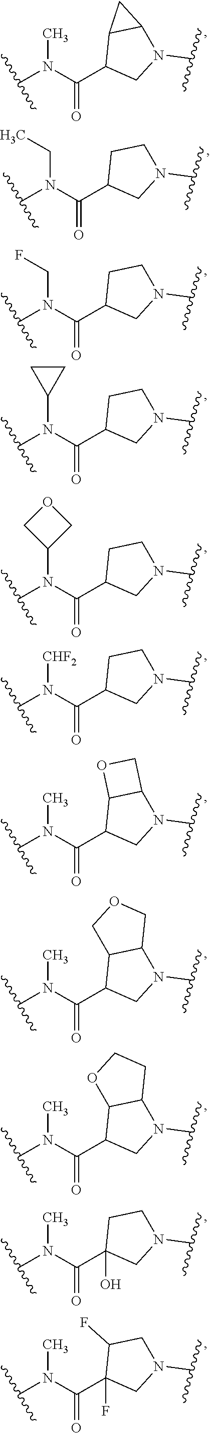

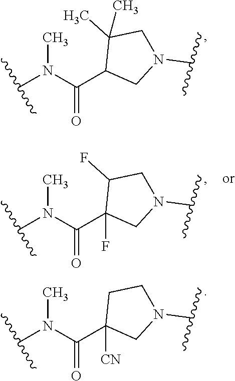

In some embodiments of Formula BI and subformula thereof, the linker has the structure:

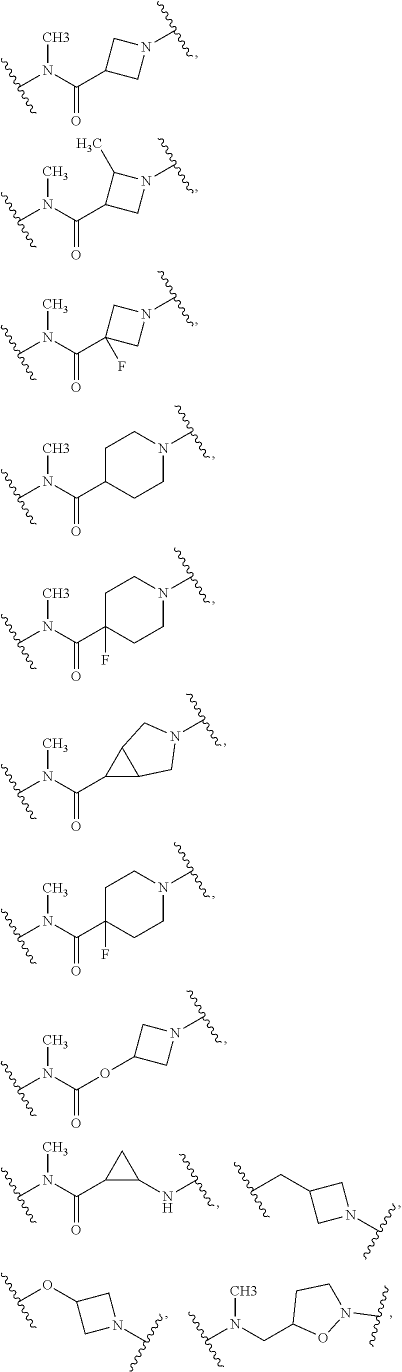

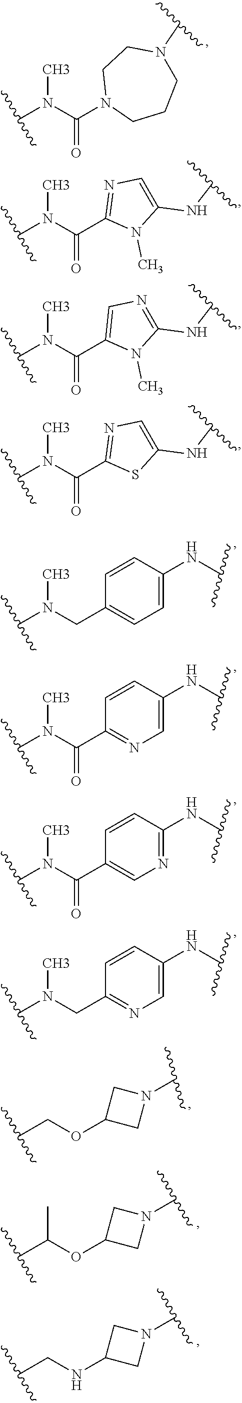

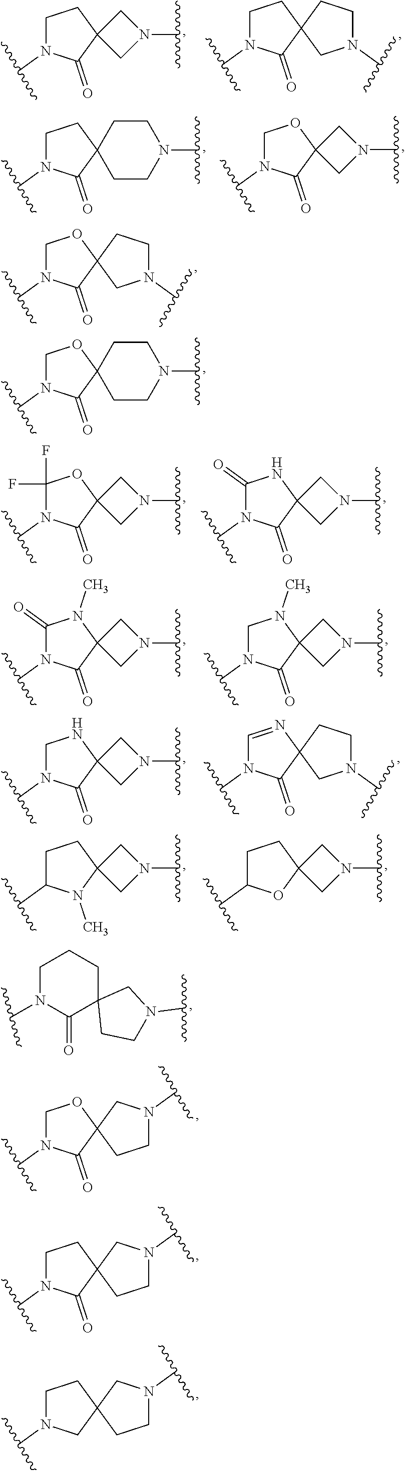

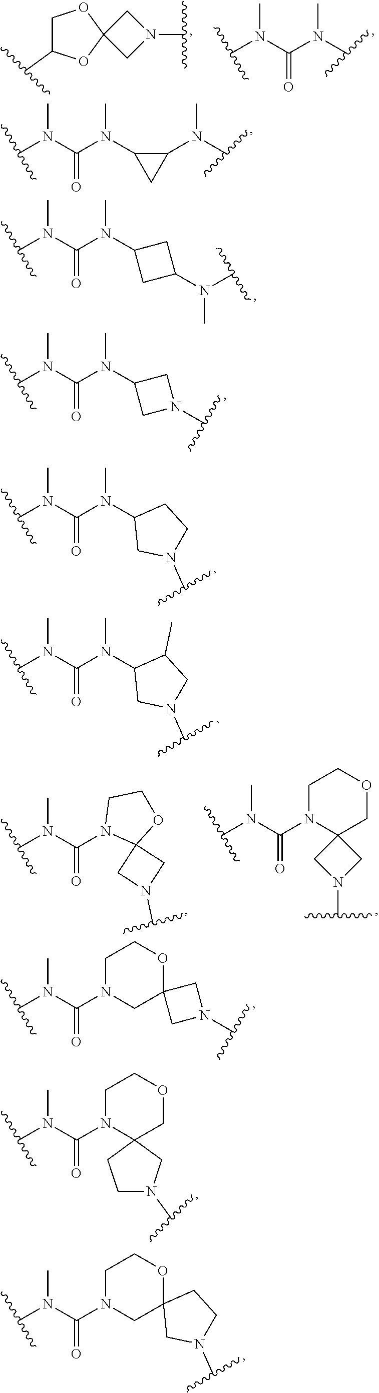

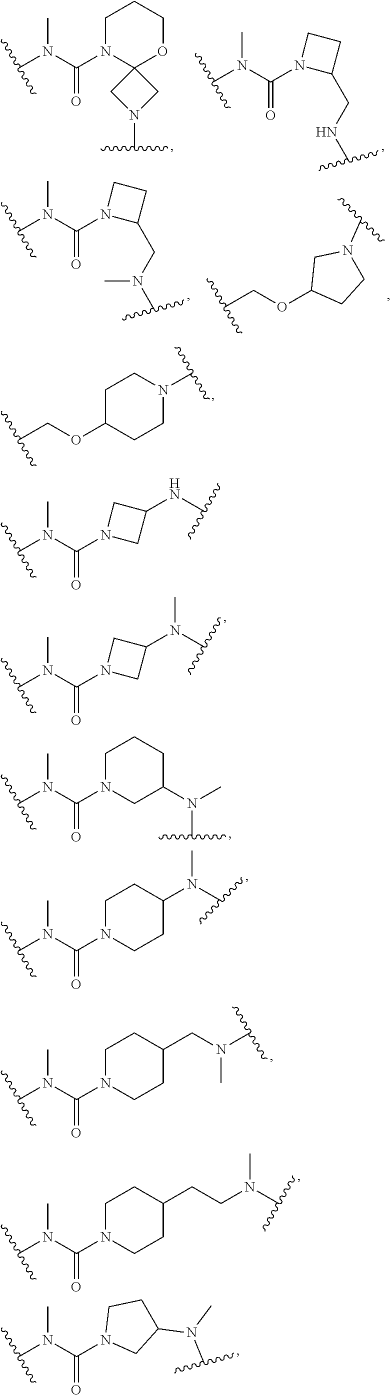

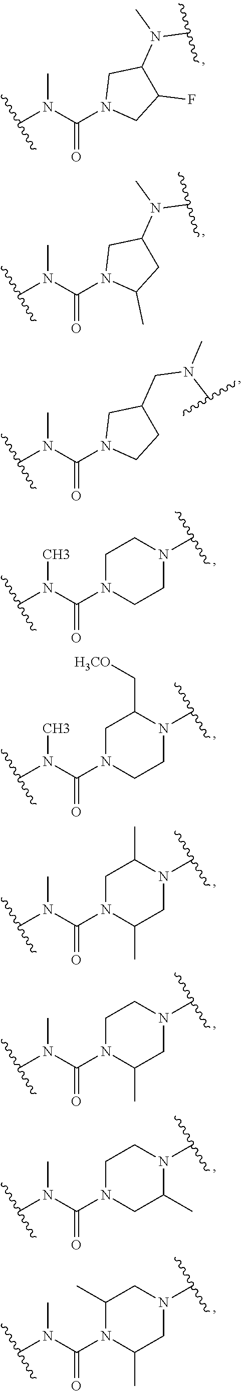

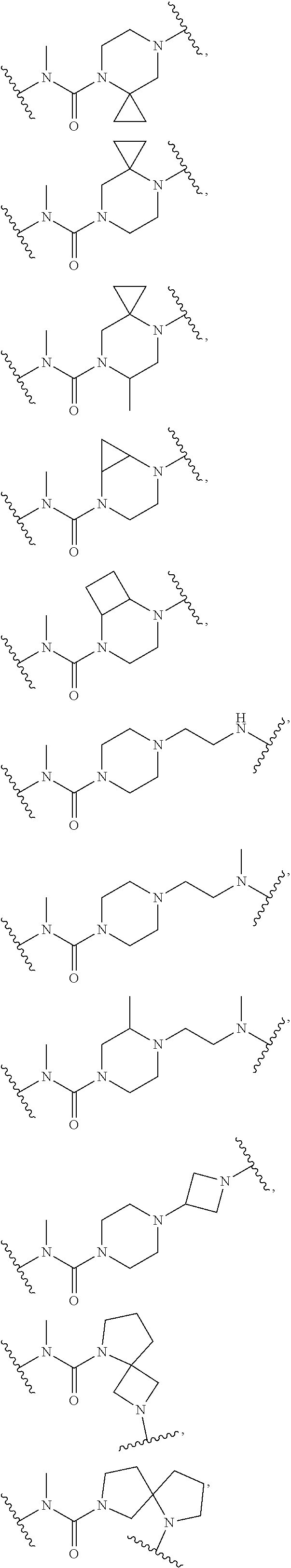

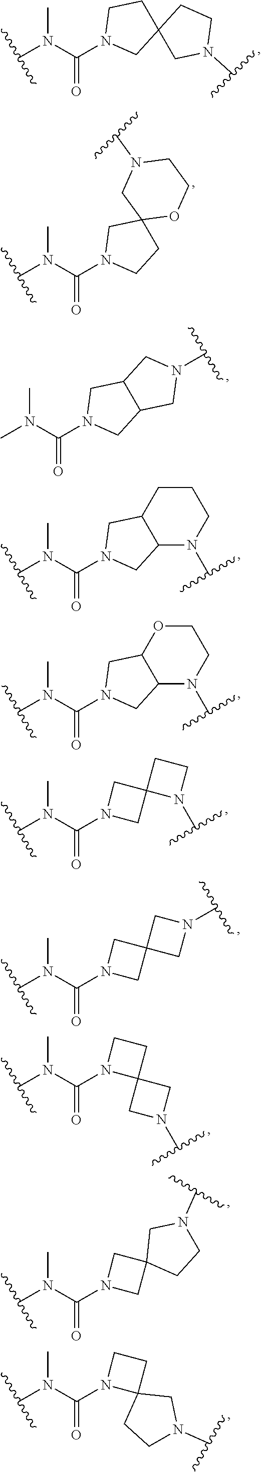

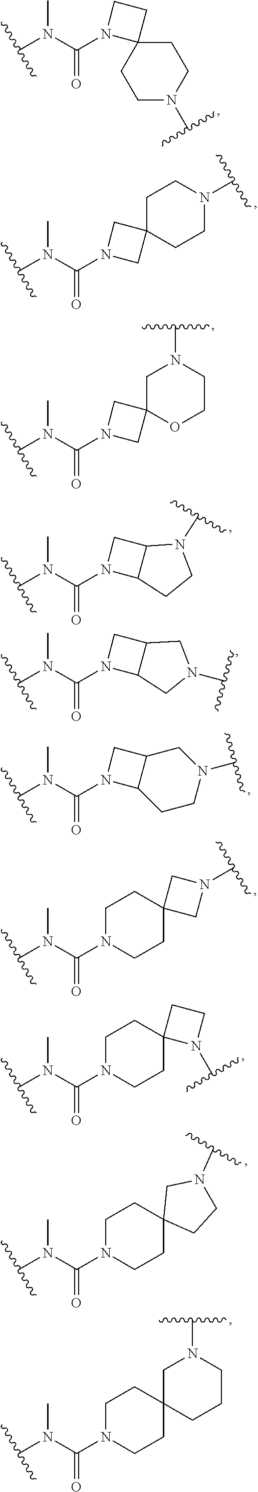

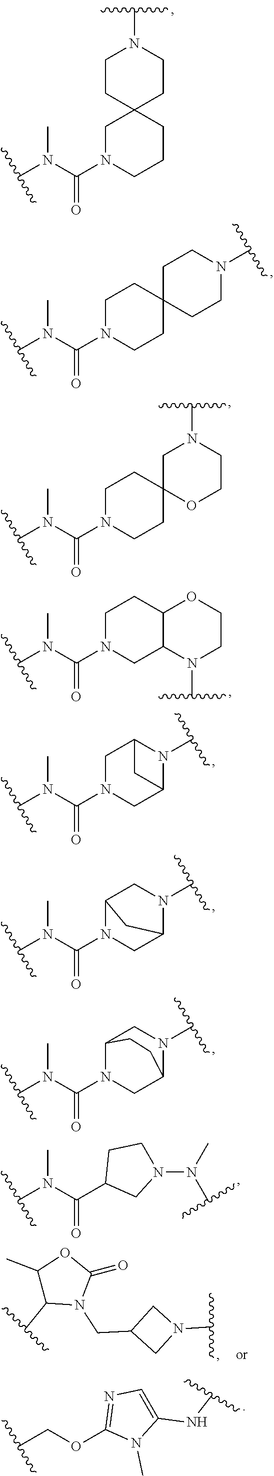

In some embodiments of Formula BI and subformula thereof, the linker has the structure

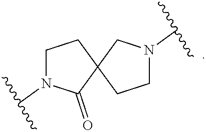 [SMILES: *N1CCC2(CCN(*)C2=O)C1]

In some embodiments of Formula BI and subformula thereof, the linker has the structure

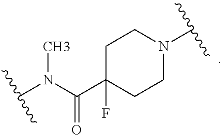 [SMILES: *N1CCC(F)(C(=O)N(*)C)CC1]

In some embodiments of Formula BI and subformula thereof, W is a cross-linking group comprising a vinyl ketone. In some embodiments, W has the structure of Formula BIIIa:

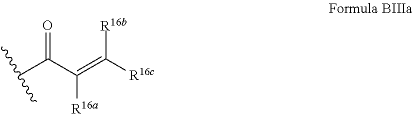

wherein R^{16a}, R^{16b}, and R^{16c} are, independently, hydrogen, —CN, halogen, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

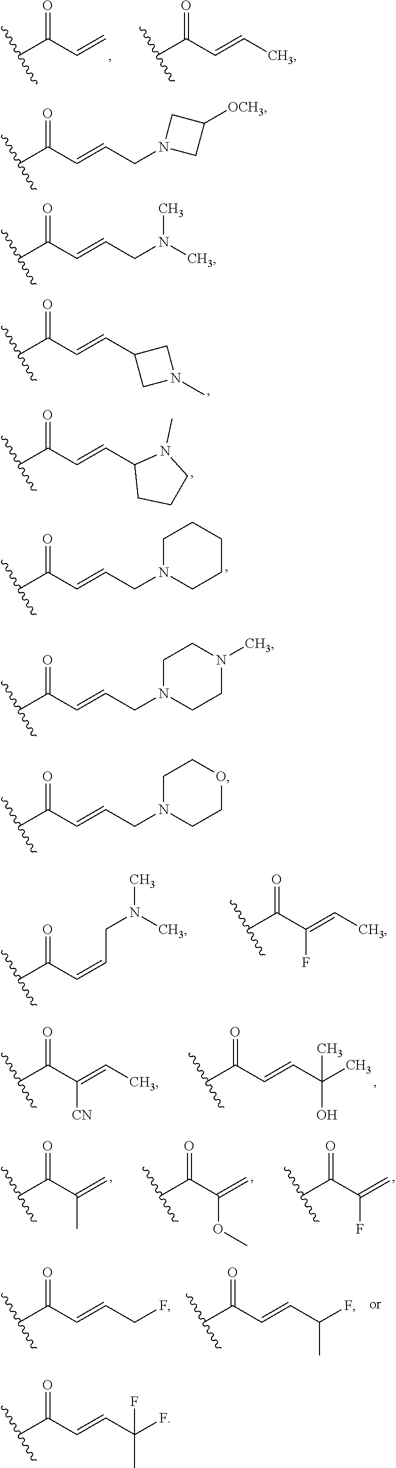

In some embodiments of Formula BI and subformula thereof, W is a cross-linking group comprising an ynone. In some embodiments, W has the structure of Formula BIIIb:

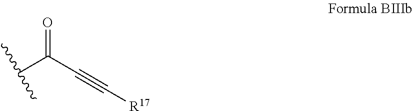

wherein R¹⁷ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl. In some embodiments, W is:

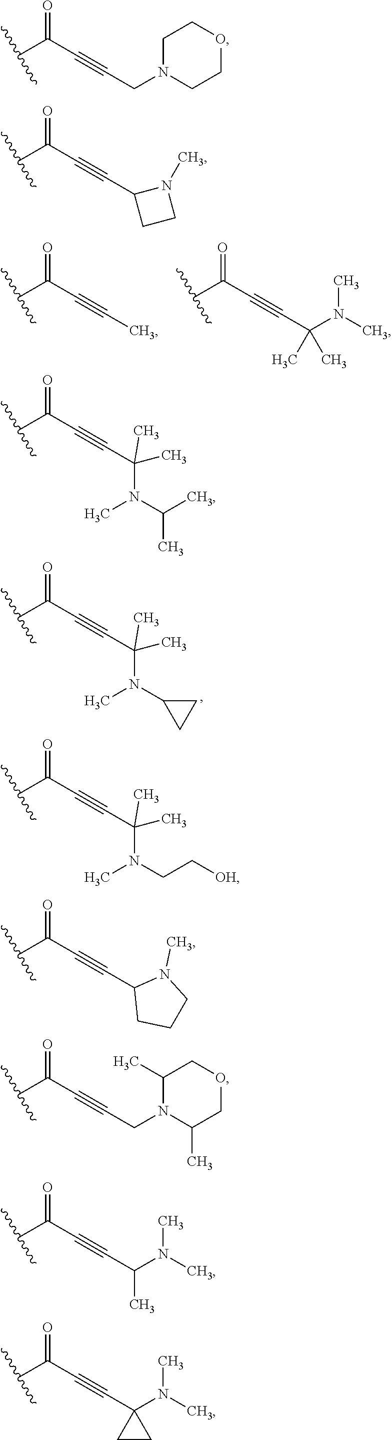

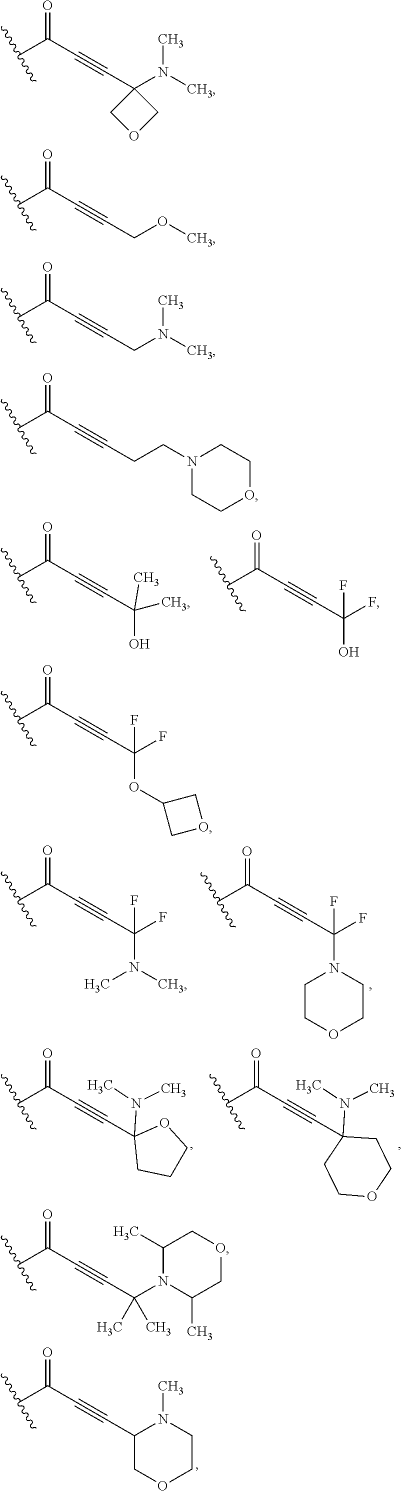

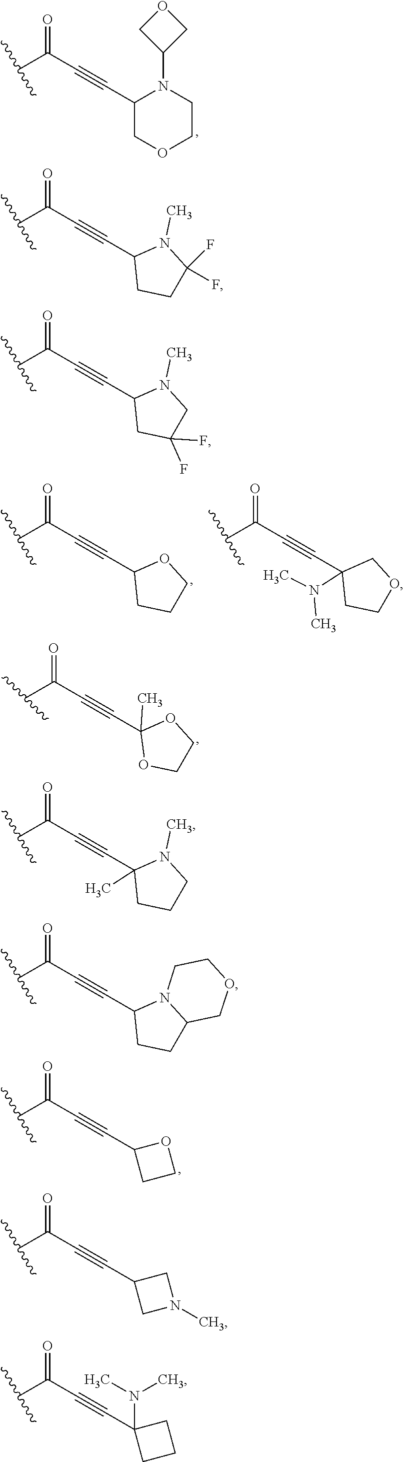

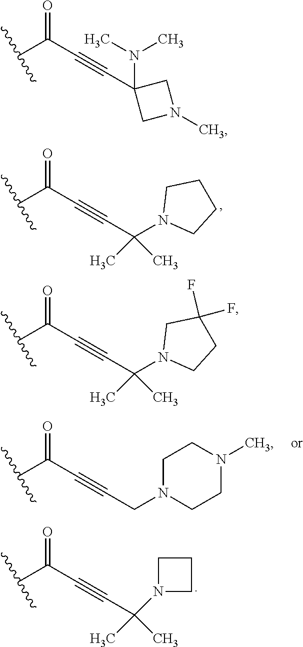

In some embodiments, W is

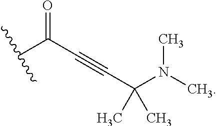 [SMILES: *C(=O)C#CC(C)(C)N(C)C]

In some embodiments of Formula BI and subformula thereof, W is a cross-linking group comprising a vinyl sulfone. In some embodiments, W has the structure of Formula BIIIc:

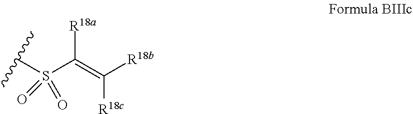

wherein R^{18a}, R^{18b}, and R^{18c} are, independently, hydrogen, —CN, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

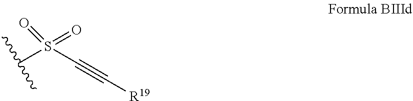

In some embodiments of Formula BI and subformula thereof, W is a cross-linking group comprising an alkynyl sulfone. In some embodiments, W has the structure of Formula BIIId:

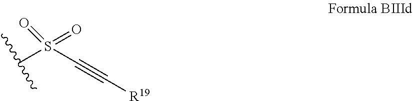

wherein R¹⁹ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl. In some embodiments, W is:

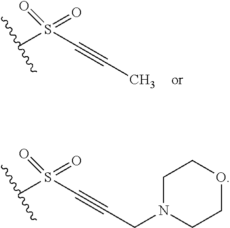

In some embodiments of Formula BI and subformula thereof, W has the structure of Formula BIIIe:

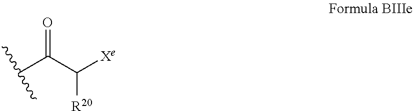

wherein X^{e} is a halogen; and
R²⁰ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments of Formula BI and subformula thereof, W is haloacetyl. In some embodiments of Formula BI and subformula thereof, W is not haloacetyl.

In some embodiments, the RAS(ON) inhibitor is selected from Table B1, or a pharmaceutically acceptable salt or stereoisomer thereof. In some embodiments, the RAS(ON) inhibitor is selected from Table B1, or a pharmaceutically acceptable salt or atropisomer thereof.

TABLE B1
Certain Compounds of the Present Invention

| Ex# | Structure |
|---|---|
| BA1 | 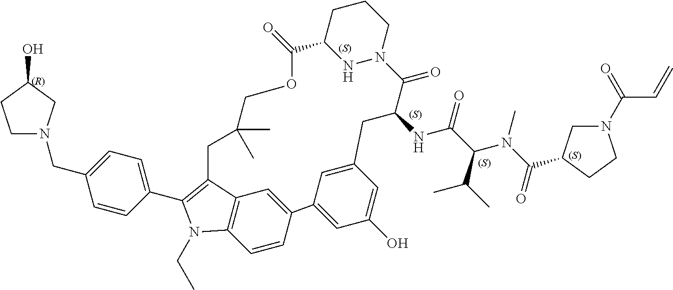 |
| BA2 | 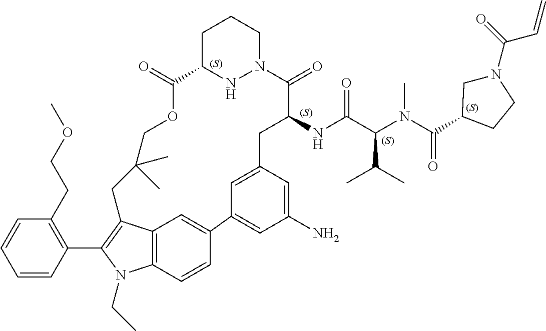 |
| BA3 | 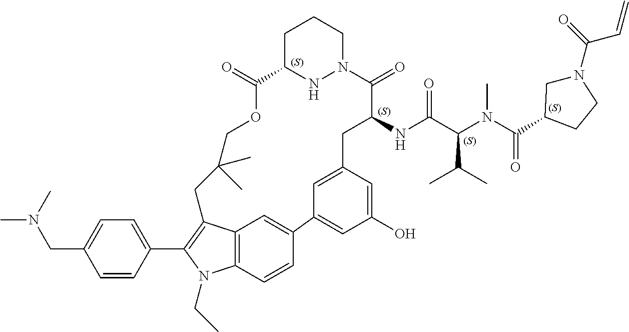 |
| BA4 | 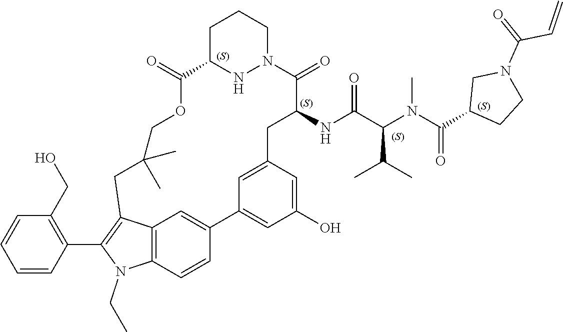 |
| BA5 | 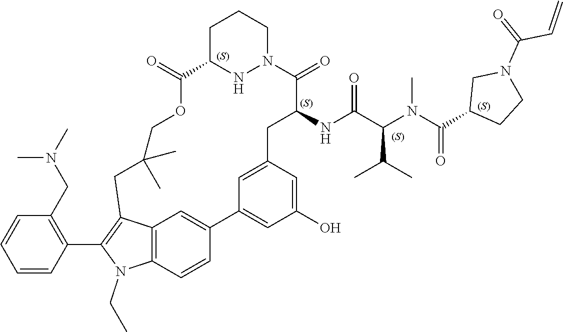 |
| BA6 | 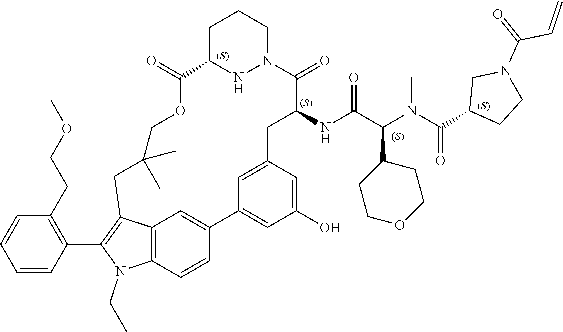 |
| BA7 | 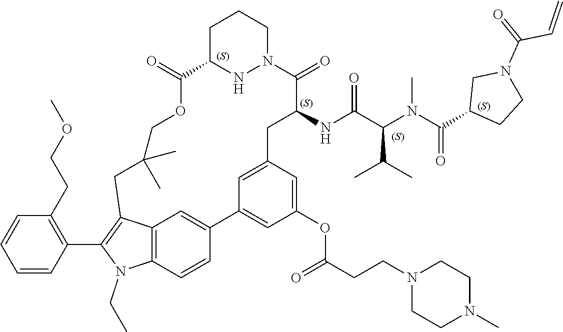 |
| BA8 | 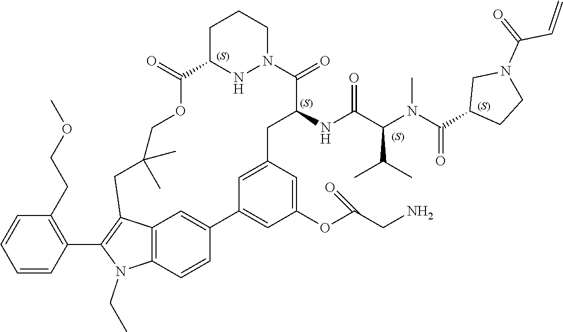 |
| BA9 | 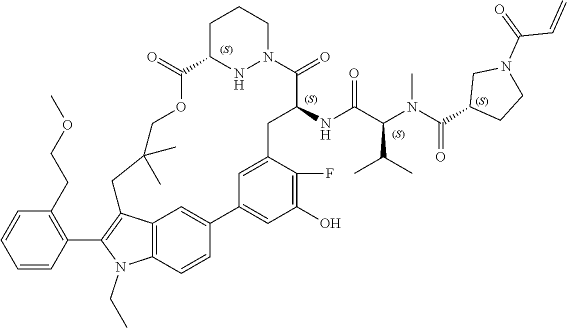 |
| BA10 | 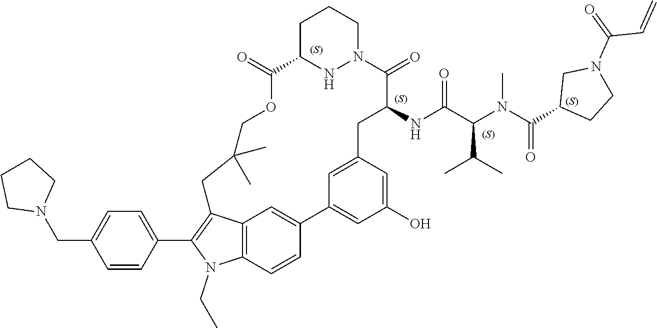 |
| BA11 | 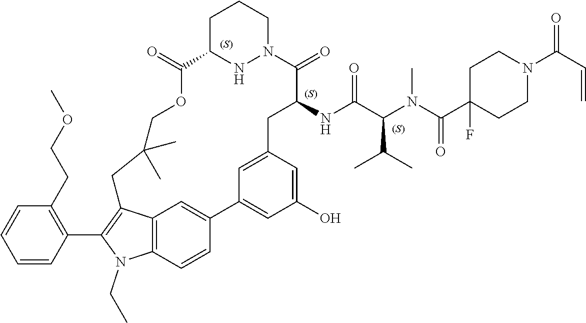 |
| BA12 | 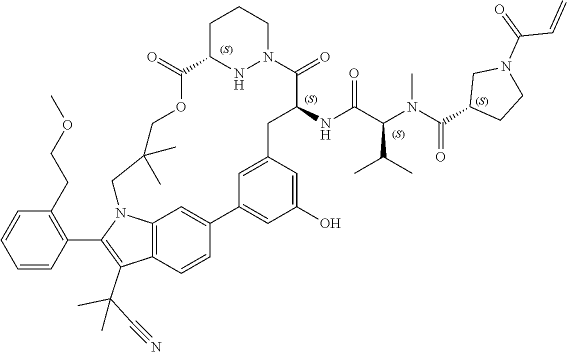 |
| BA13 | 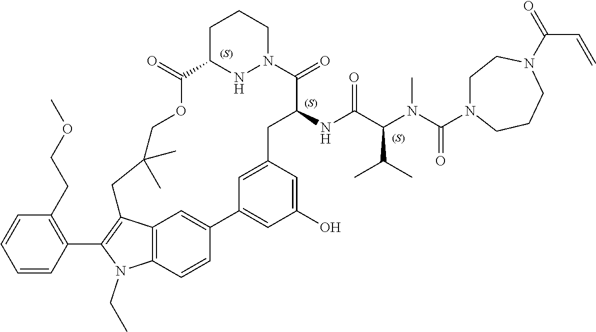 |
| BA14 | 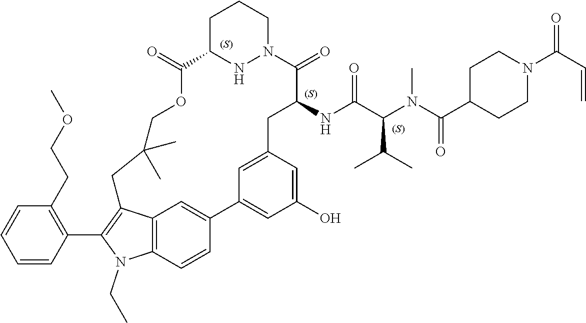 |
| BA15 | 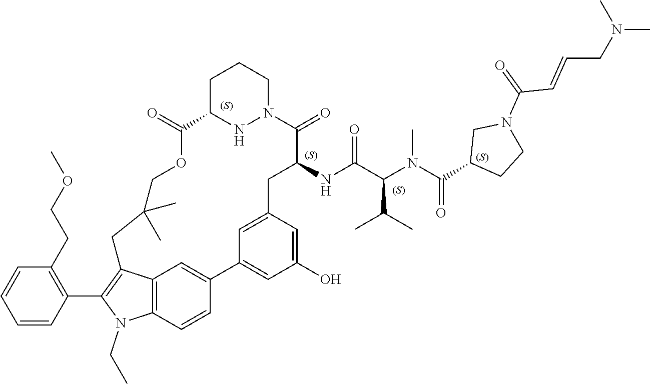 |
| BA16 | 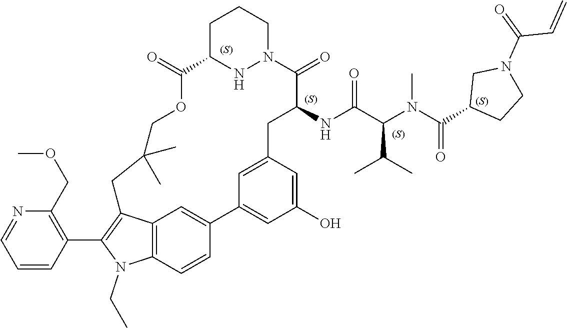 |
| BA17 | 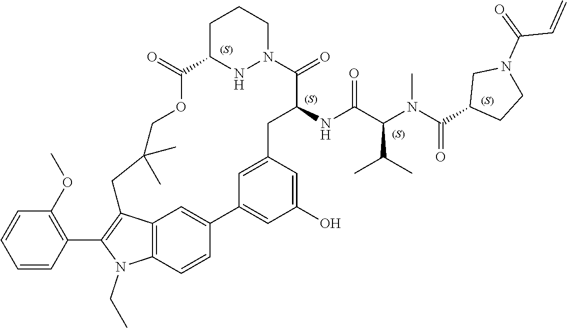 |
| BA18 | 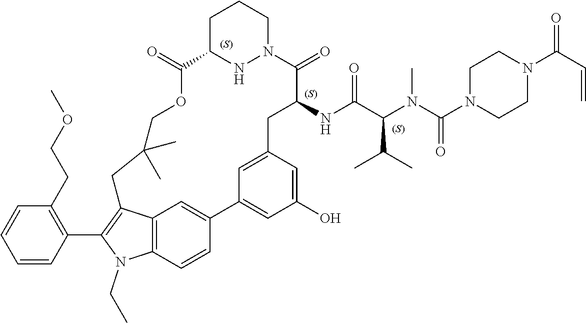 |
| BA19 | 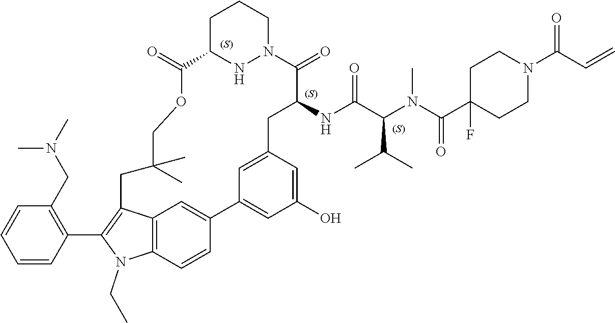 |
| BA20 | 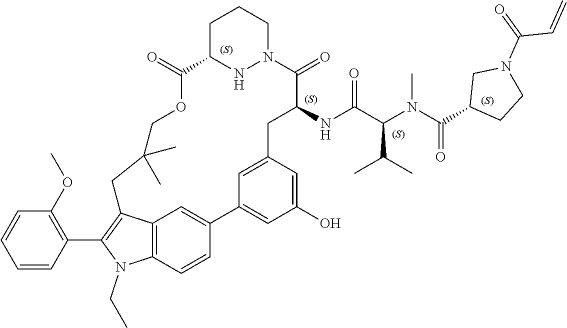 |
| BA21 | 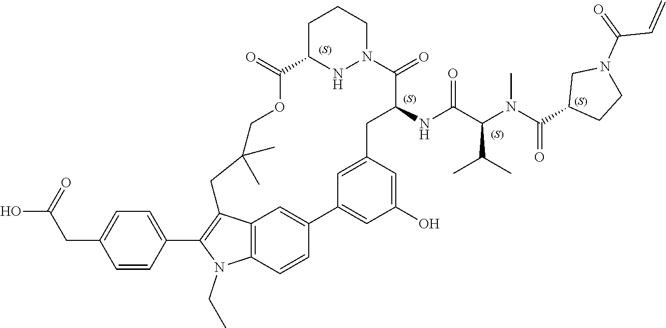 |
| BA22 | 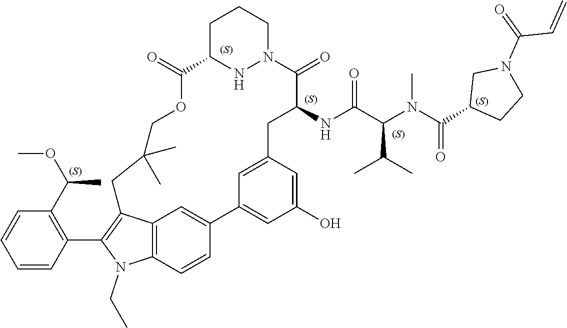 |
| BA23 | 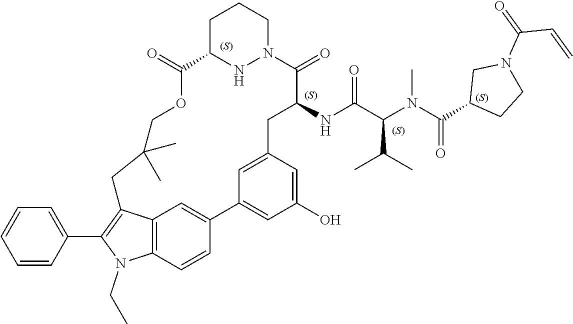 |
| BA24 | 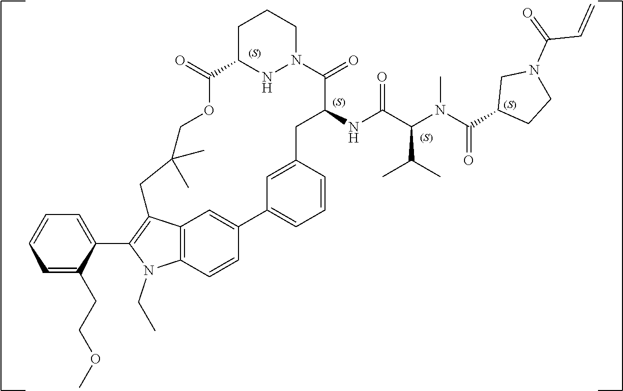 |
| BA25 | 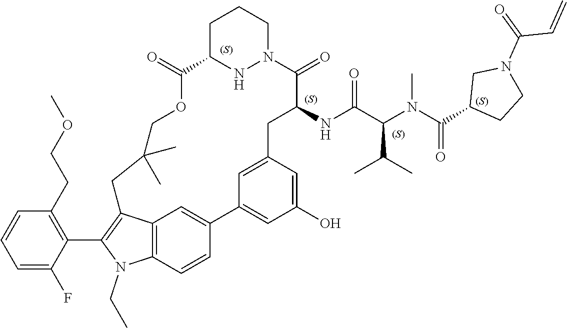 |
| BA26 | 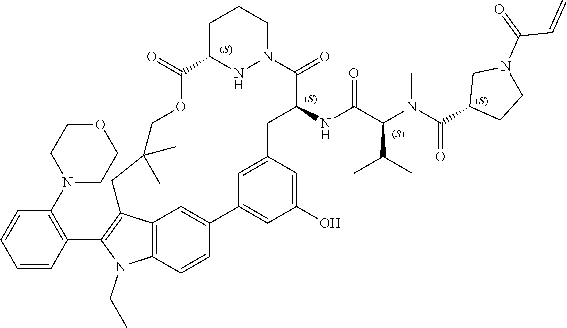 |
| BA27 | 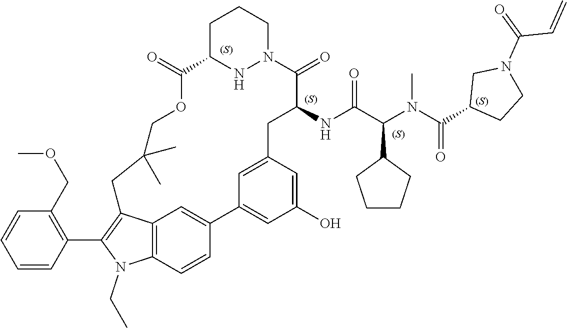 |
| BA28 | 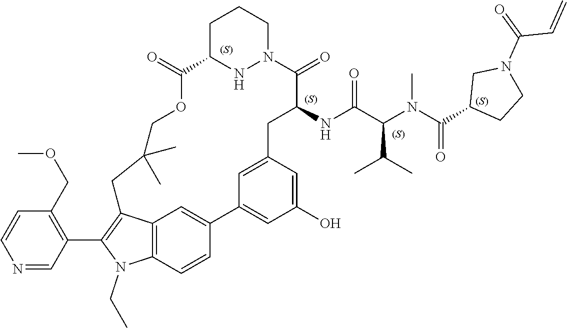 |
| BA29 | 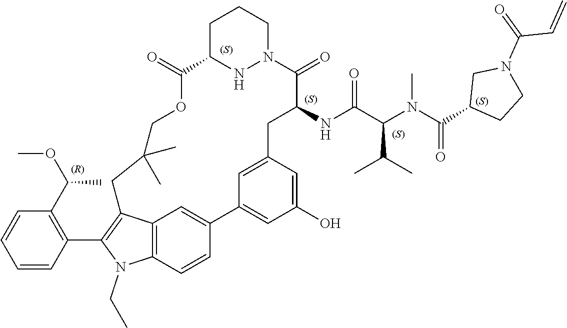 |
| BA30 | 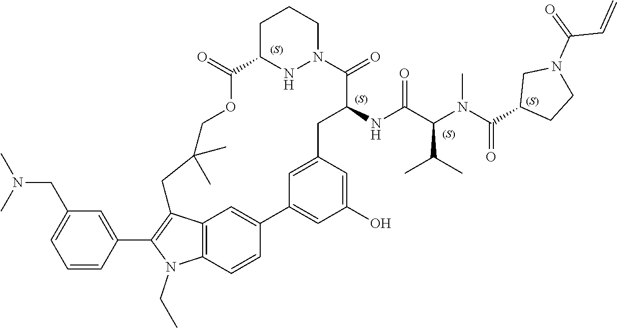 |
| BA31 | 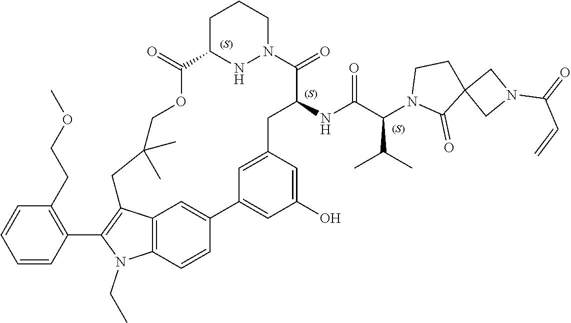 |
| BA32 | 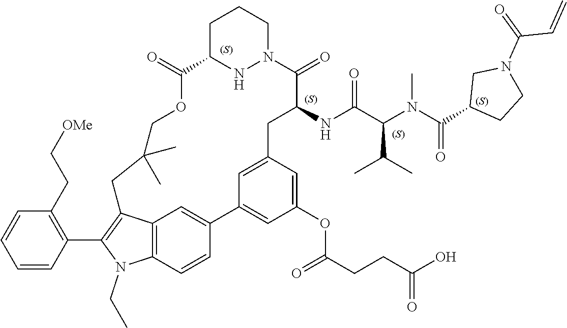 |
| BA33 | 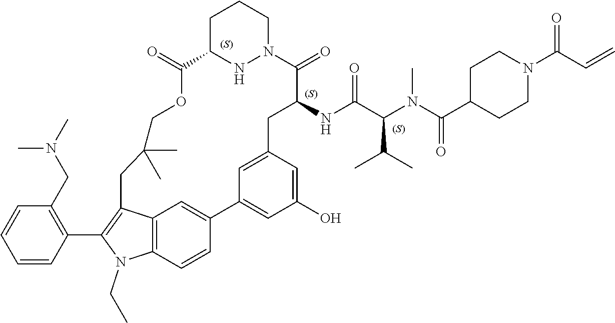 |
| BA34 | 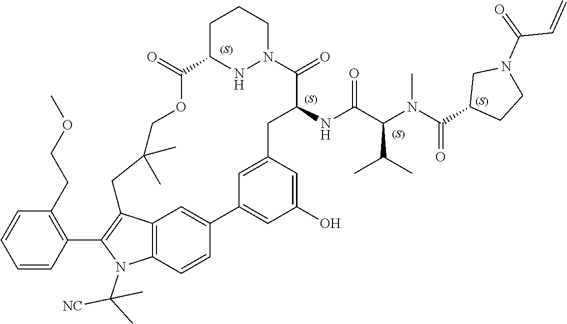 |
| BA35 | 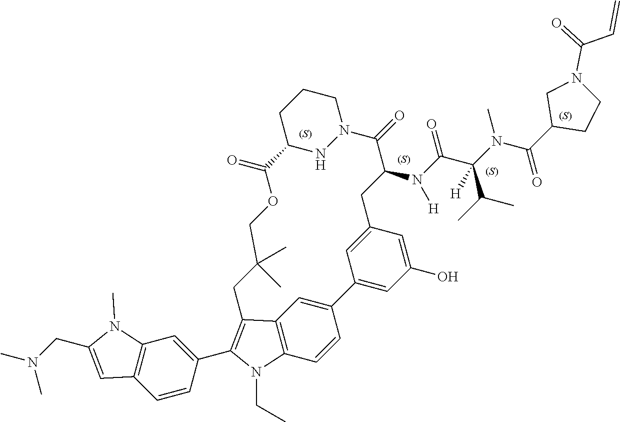 |
| BA36 | 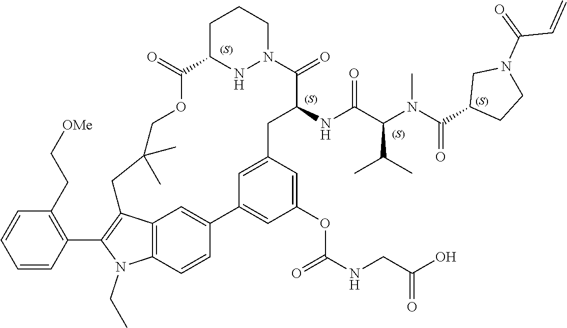 |
| BA37 | 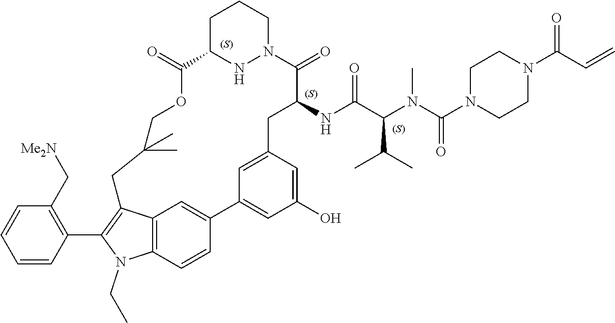 |
| BA38 | 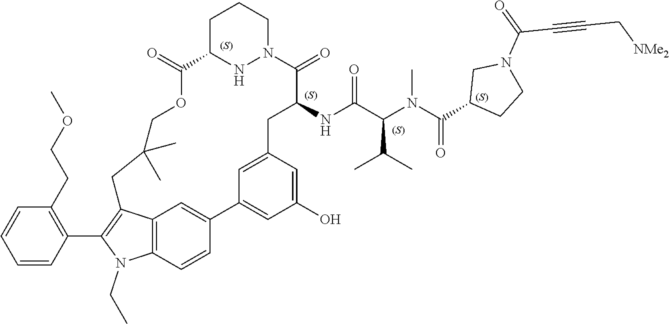 |
| BA39 | 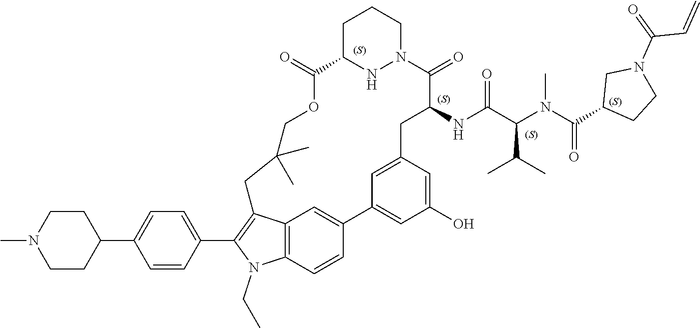 |
| BA40 | 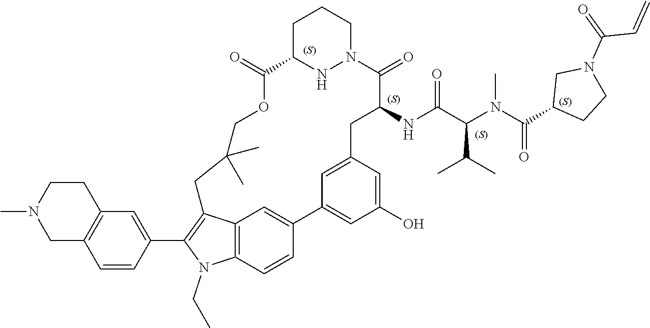 |
| BA41 | 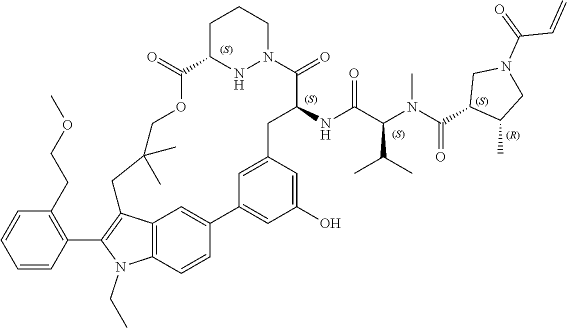 |
| BA42 | 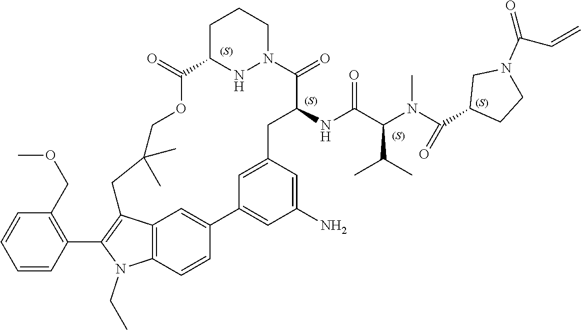 |
| BA43 | 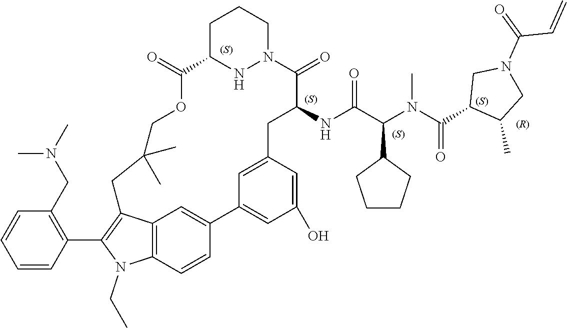 |
| BA44 | 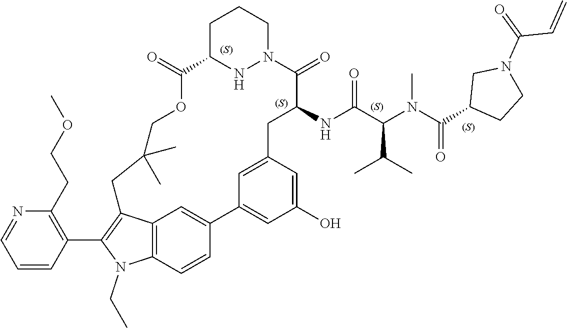 |
| BA45 | 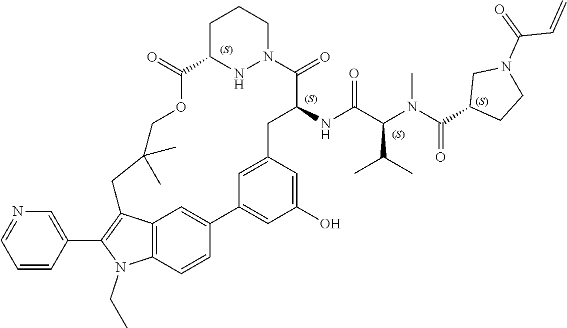 |
| BA46 | 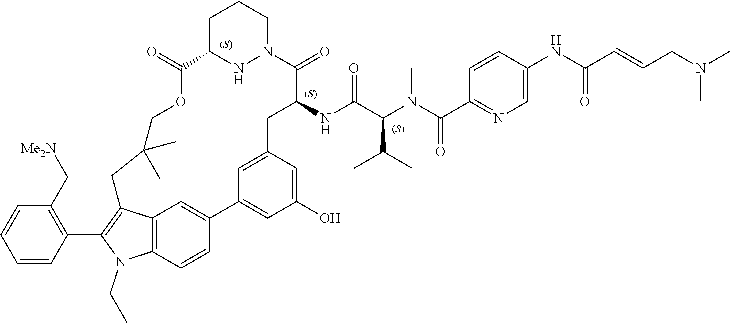 |
| BA47 | 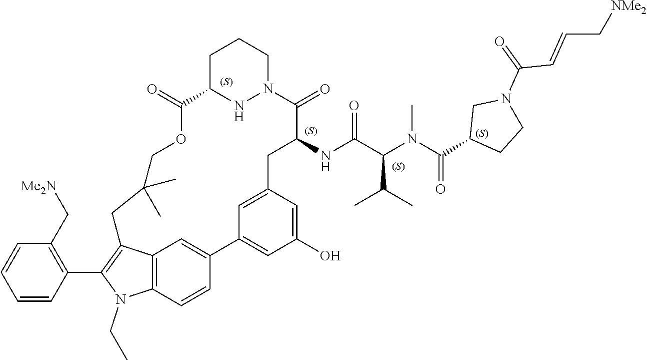 |
| BA48 | 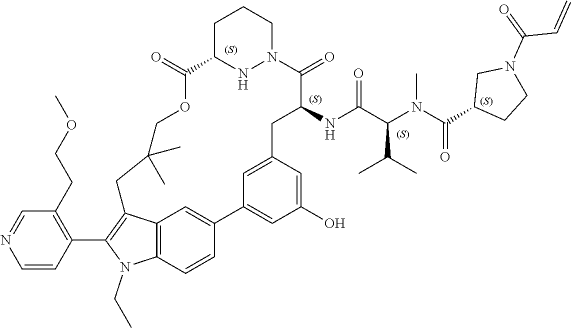 |
| BA49 | 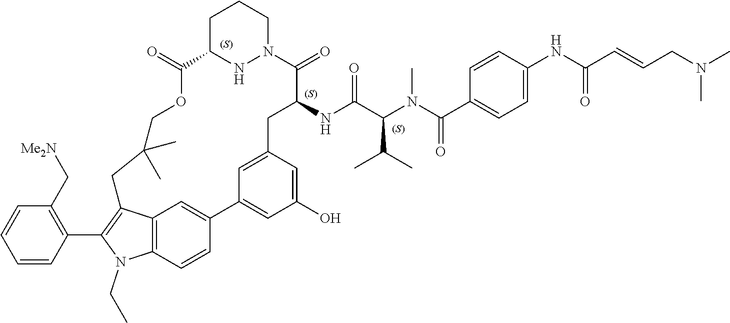 |
| BA50 | 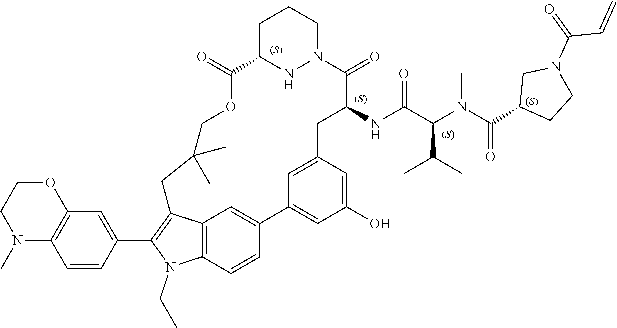 |
| BA51 | 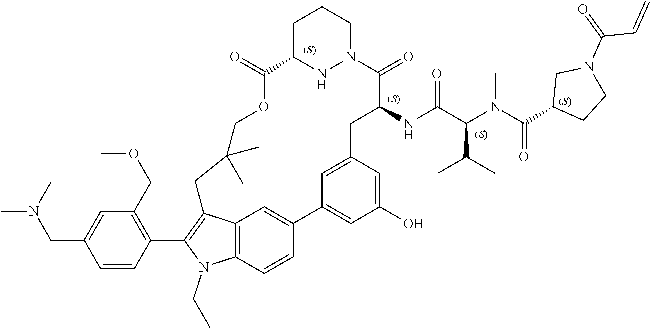 |
| BA52 | 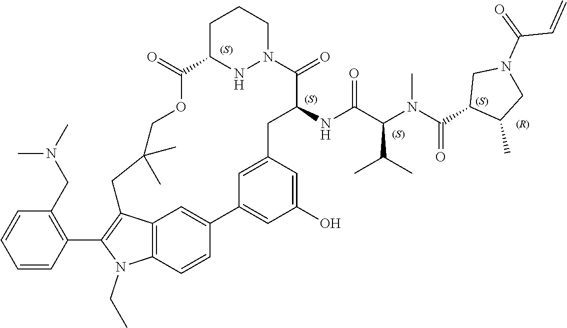 |
| BA53 | 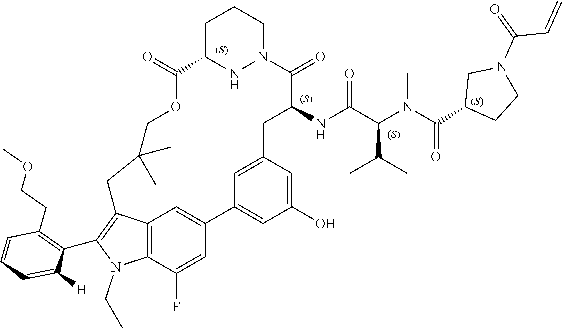 |
| BA54 | 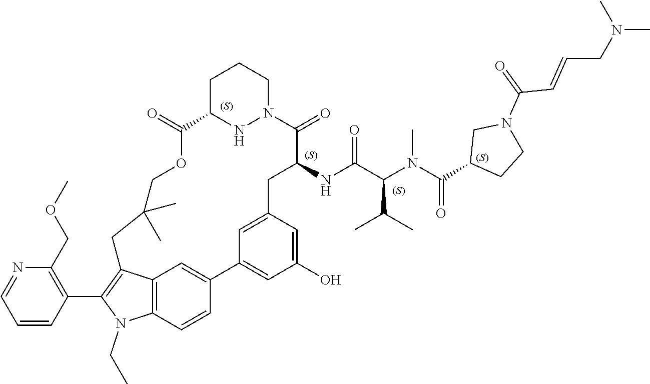 |
| BA55 | 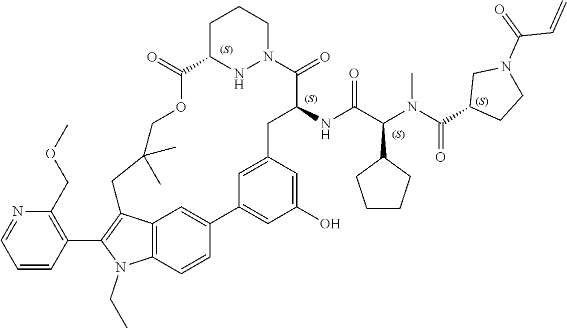 |
| BA56 | 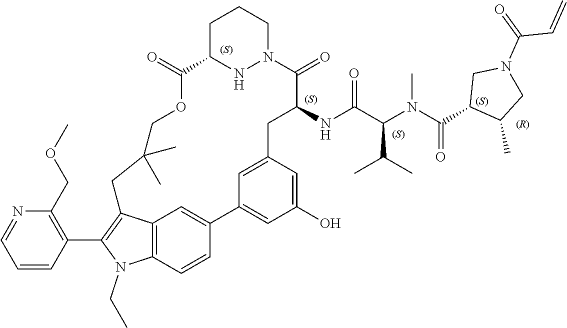 |
| BA57 | 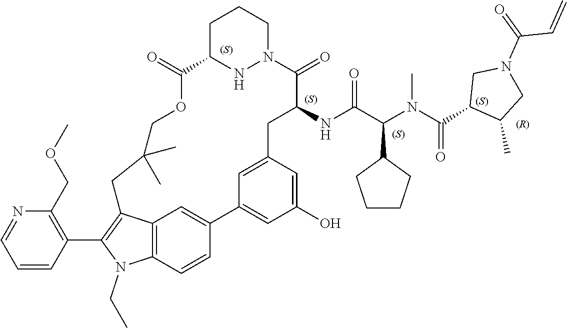 |
| BA58 | 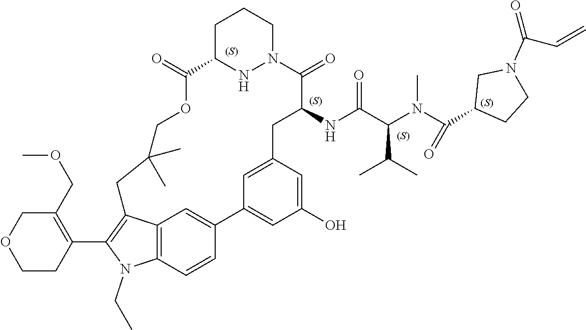 |
| BA59 | 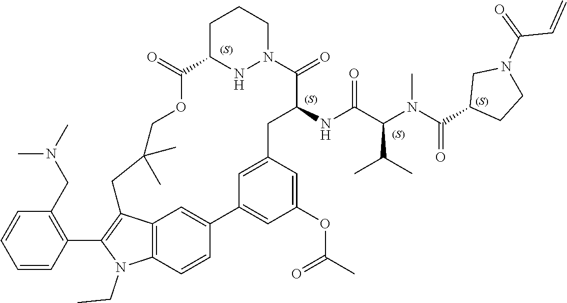 |
| BA60 | 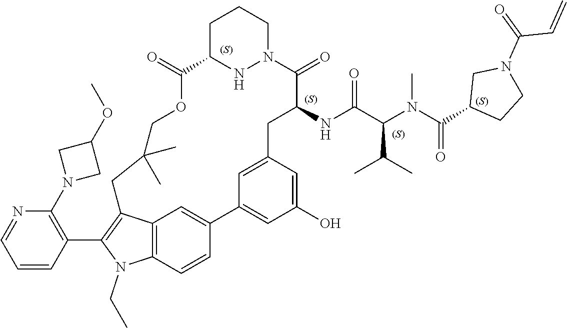 |
| BA61 | 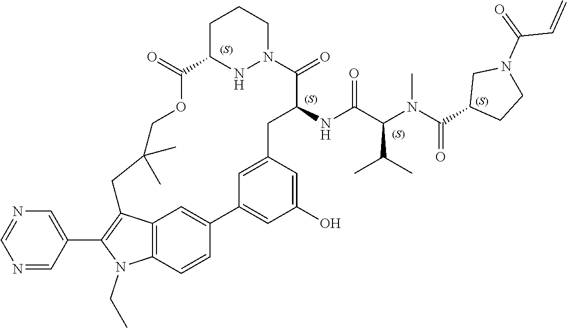 |
| BA62 | 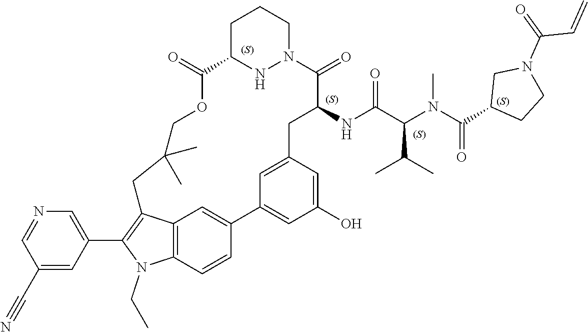 |
| BA63 | 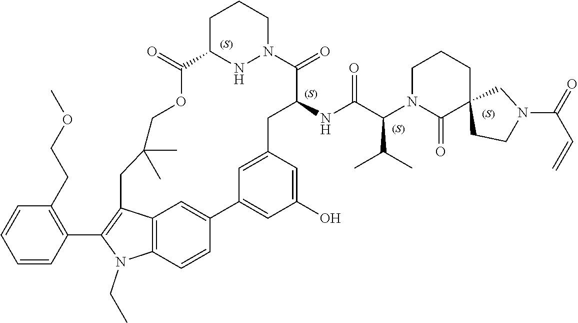 |
| BA64 | 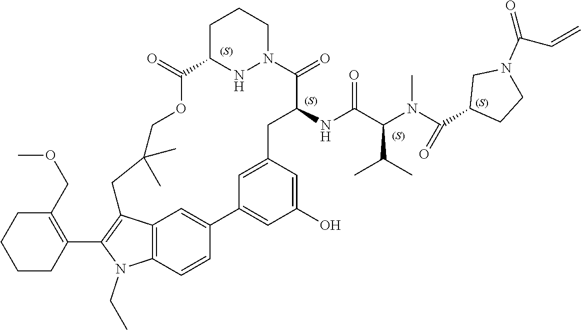 |
| BA65 | 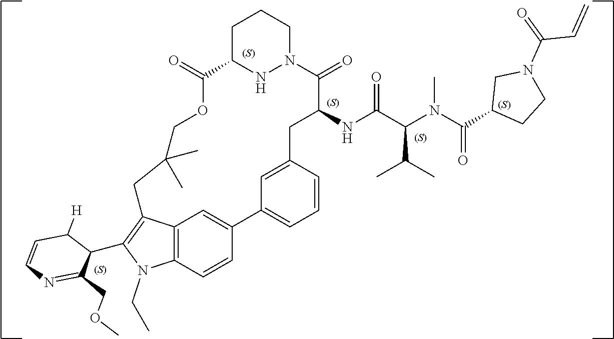 |
| BA66 | 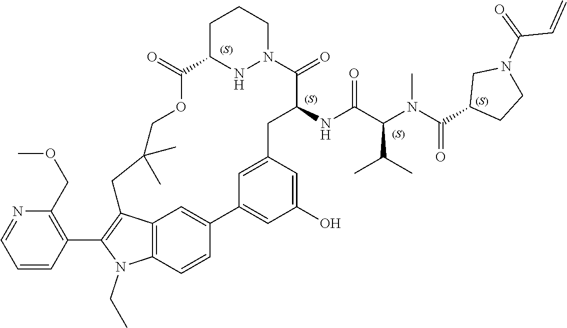 |
| BA67 | 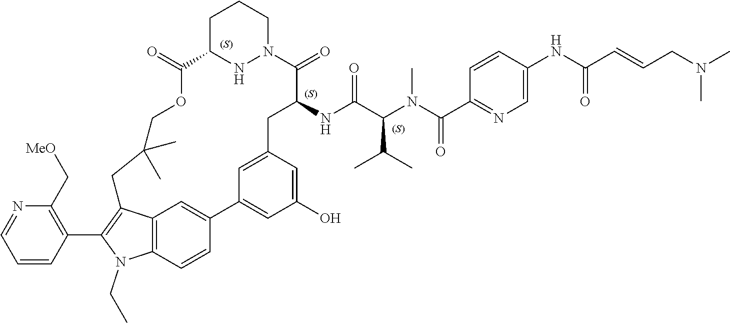 |
| BA68 | 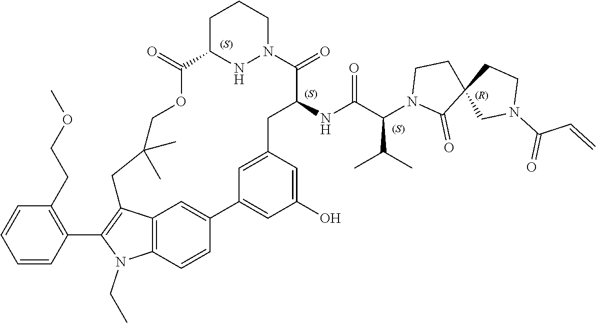 |
| BA69 | 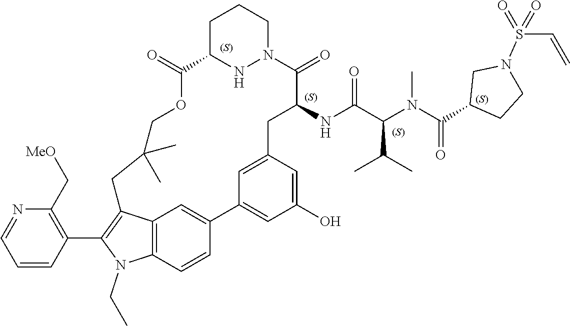 |
| BA70 | 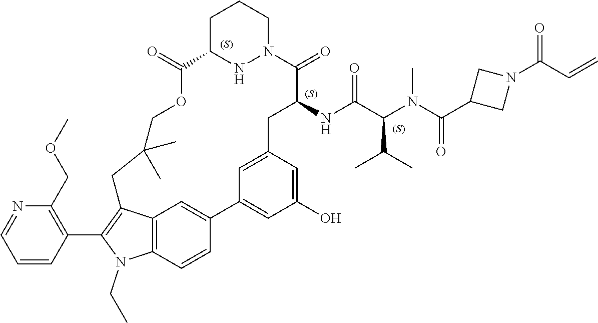 |
| BA71 | 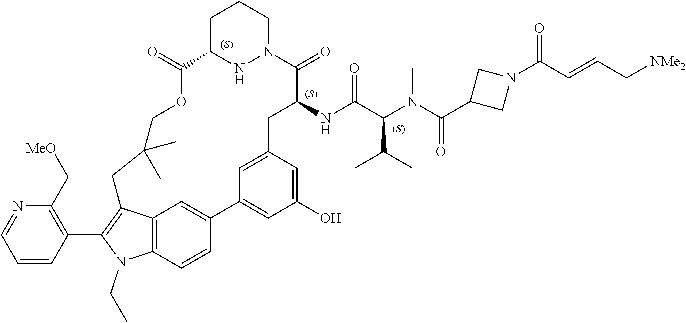 |
| BA72 | 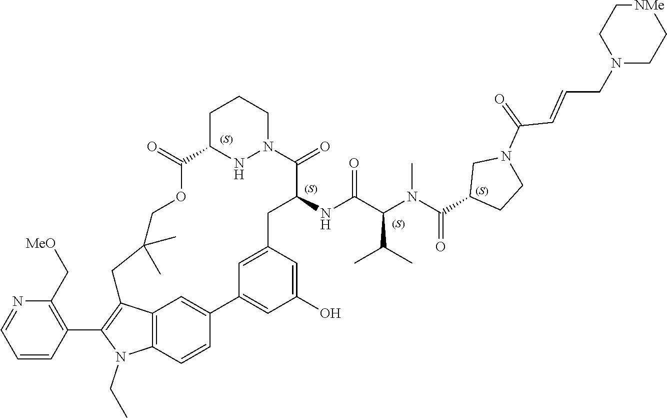 |
| BA73 | 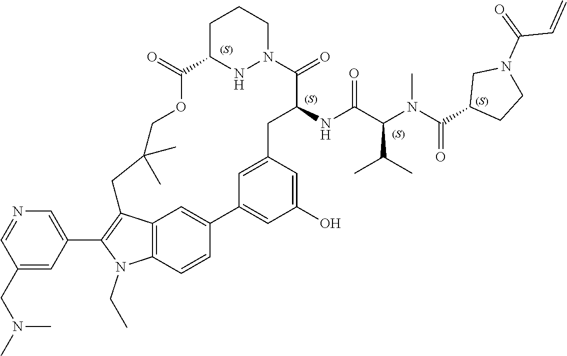 |
| BA74 | 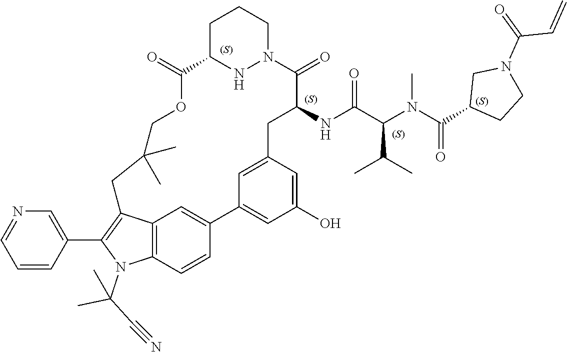 |
| BA75 | 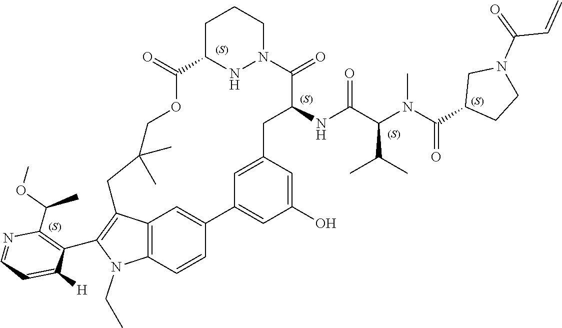 |
| BA76 | 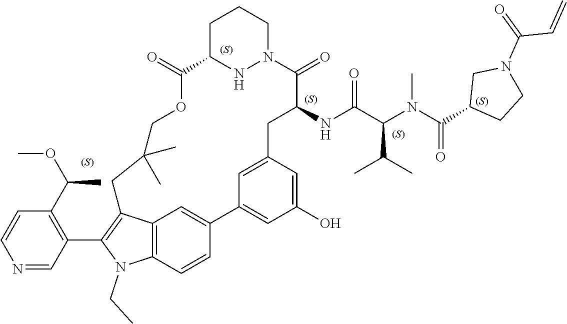 |
| BA77 | 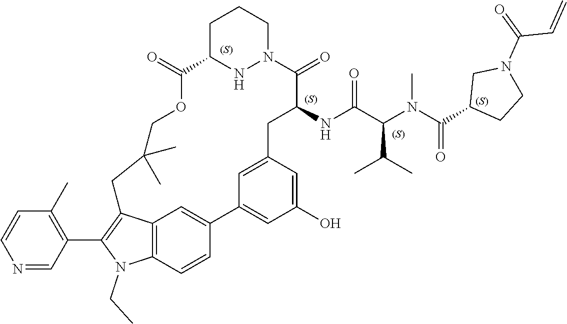 |
| BA78 | 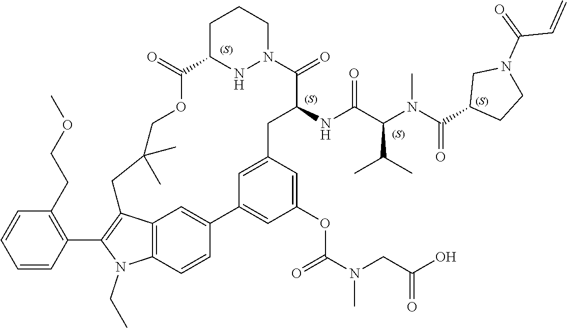 |
| BA79 | 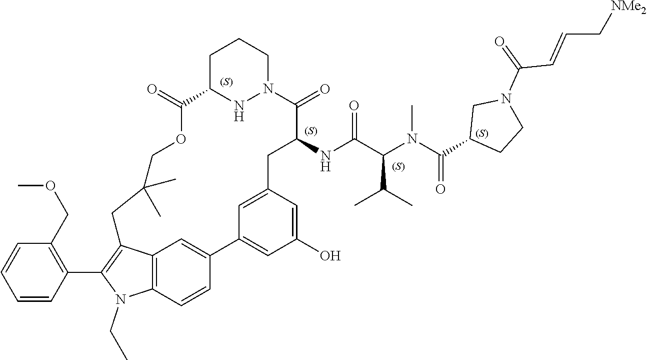 |
| BA80 | 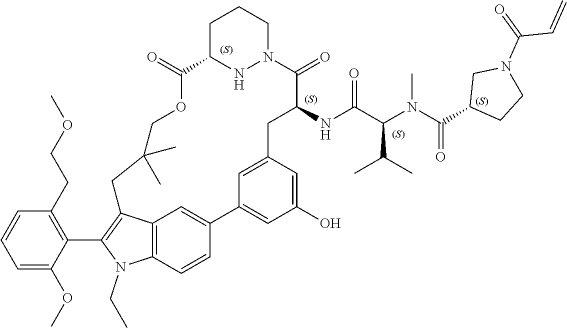 |
| BA81 | 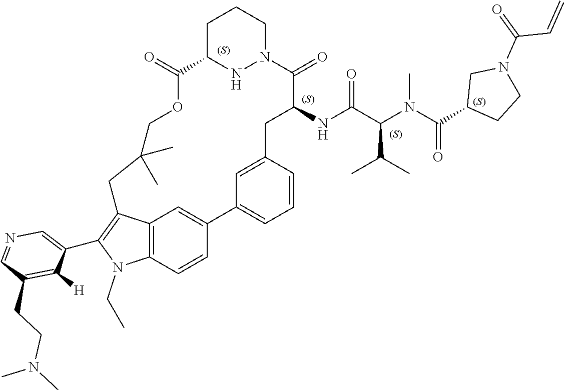 |
| BA82 | 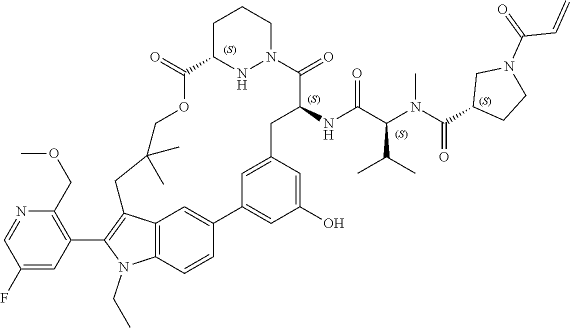 |
| BA83 | 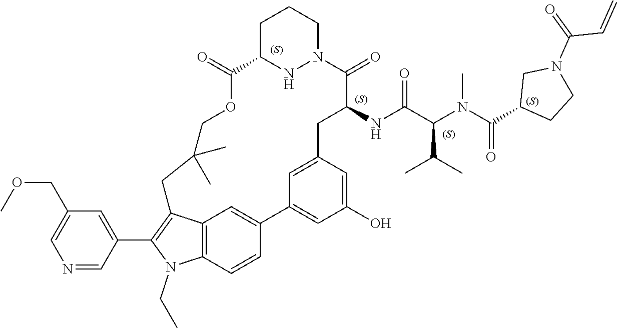 |
| BA84 | 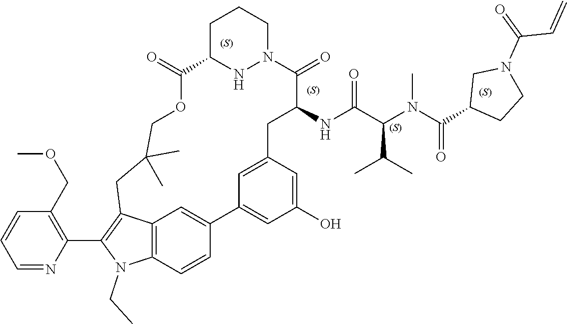 |
| BA85 | 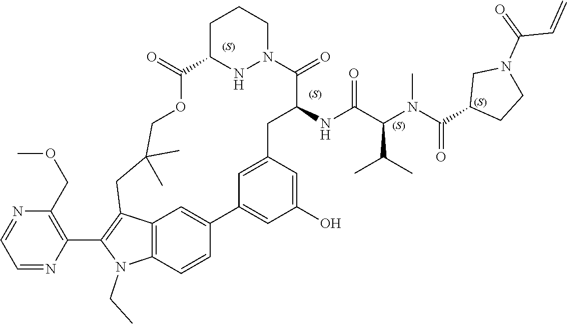 |
| BA86 | 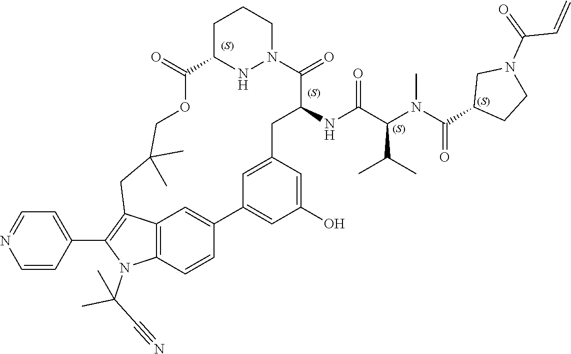 |
| BA87 | 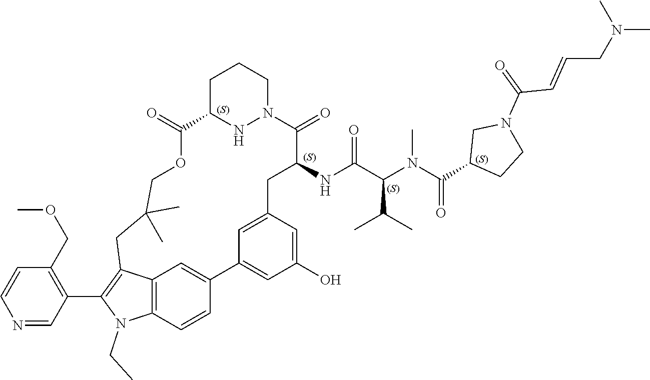 |
| BA88 | 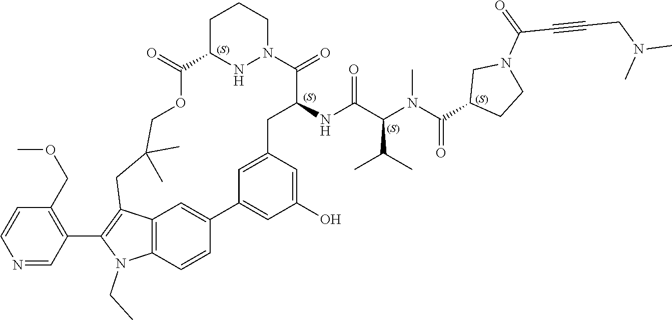 |
| BA89 | 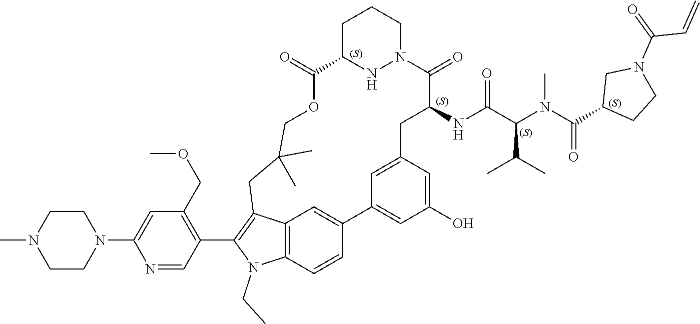 |
| BA90 | 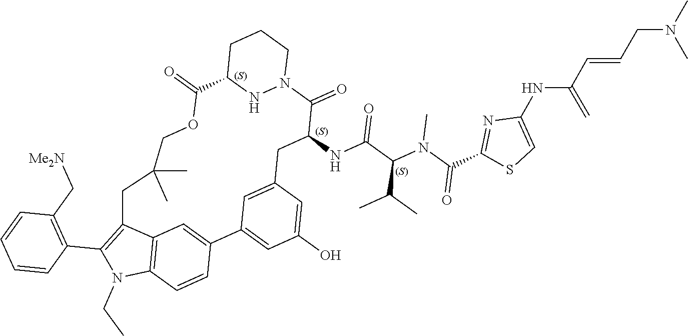 |
| BA91 | 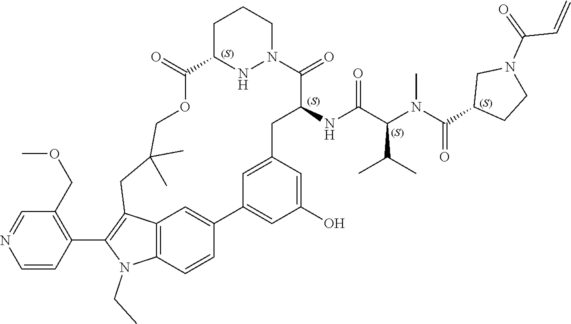 |
| BA92 | 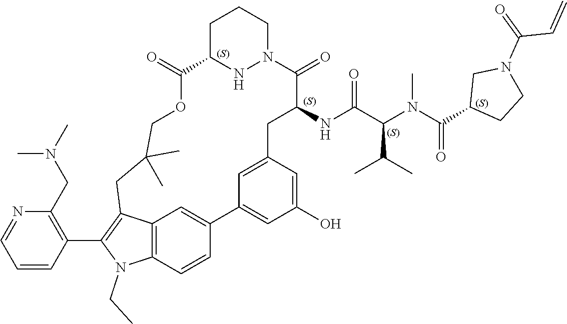 |
| BA93 | 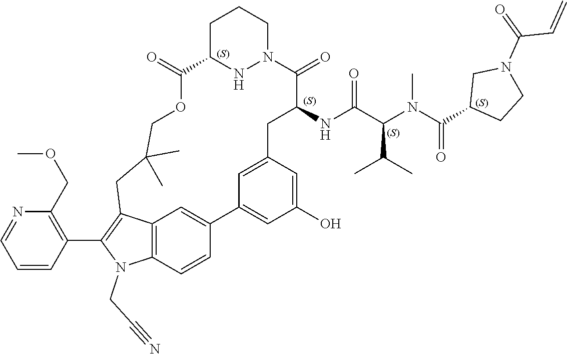 |
| BA94 | 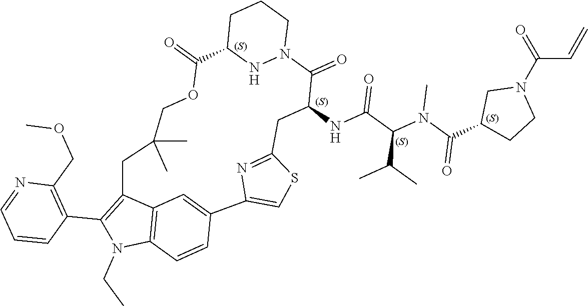 |
| BA95 | 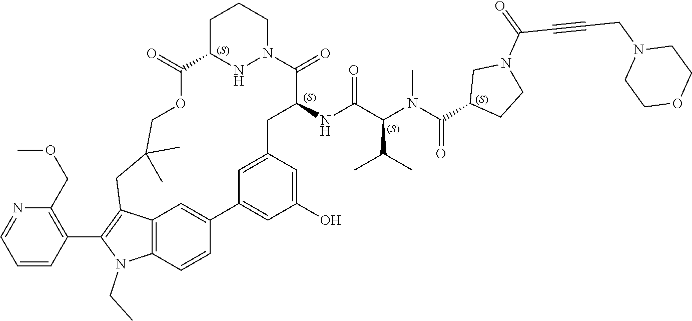 |
| BA96 | 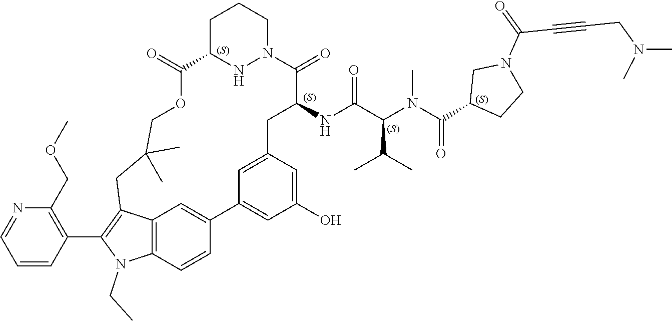 |
| BA97 | 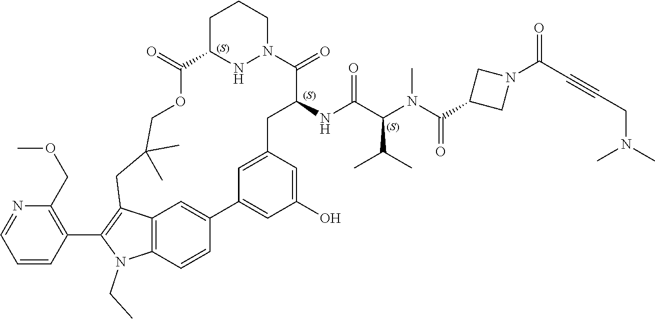 |
| BA98 | 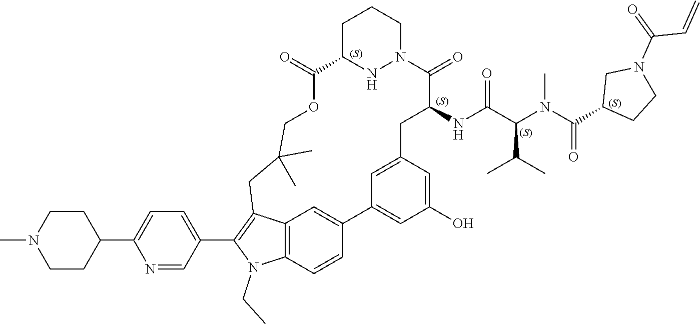 |
| BA99 | 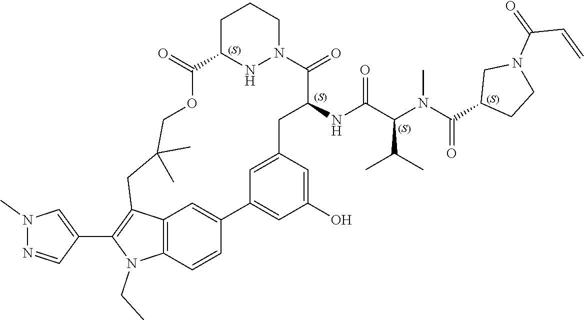 |
| BA100 | 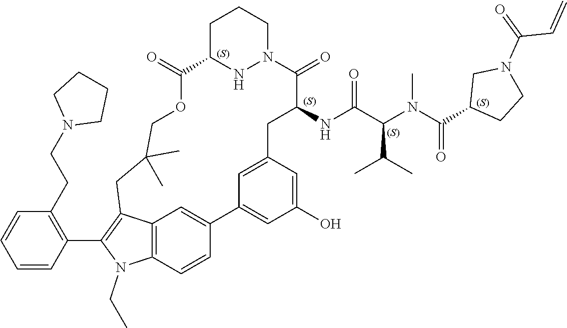 |
| BA101 | 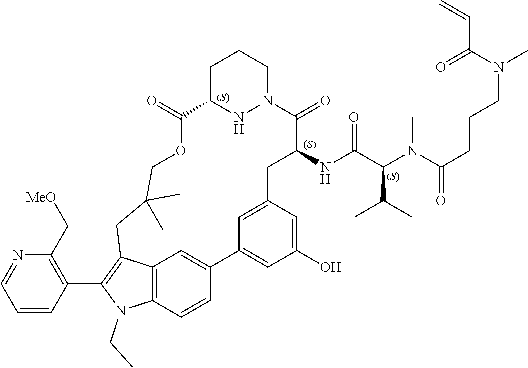 |
| BA102 | 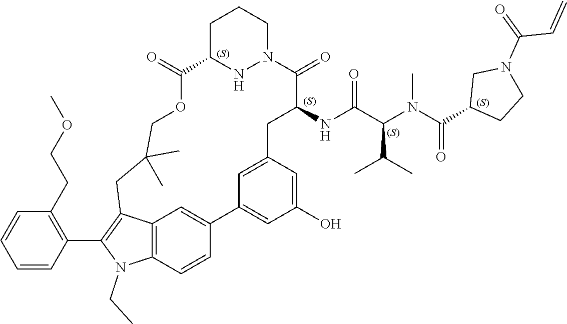 |
| BA103 | 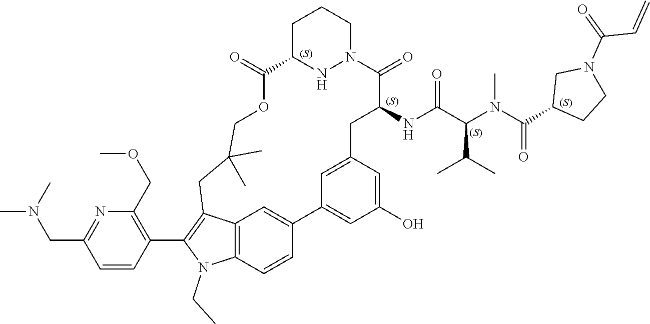 |
| BA104 | 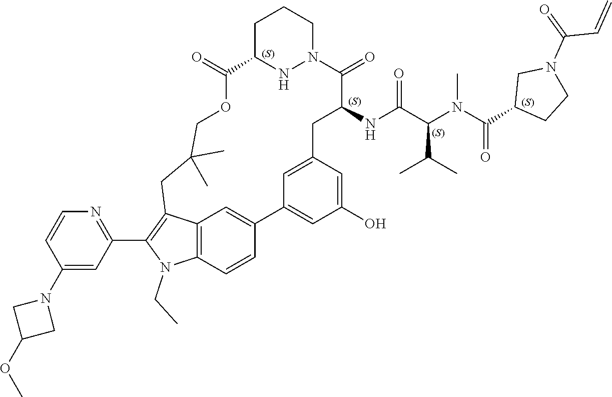 |
| BA105 | 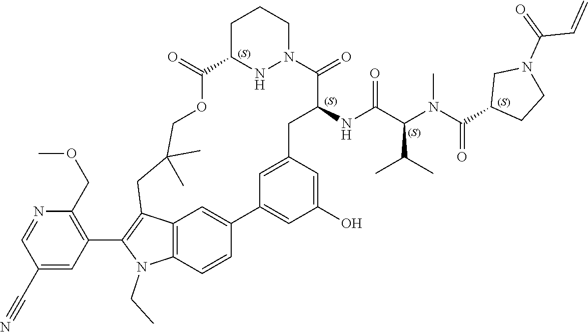 |
| BA106 | 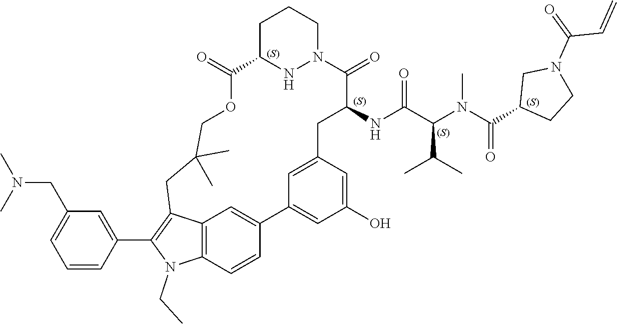 |
| BA107 | 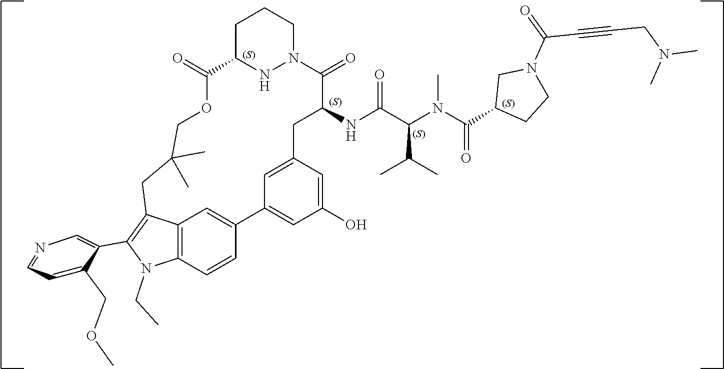 |
| BA108 | 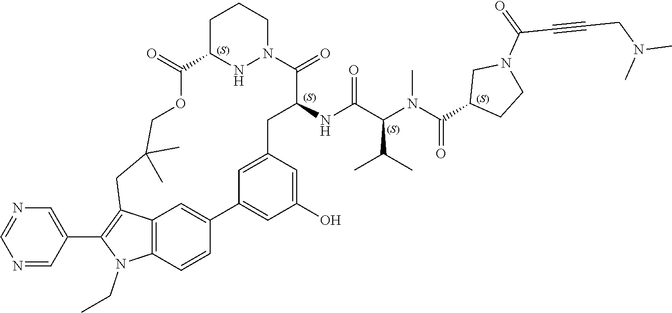 |
| BA109 | 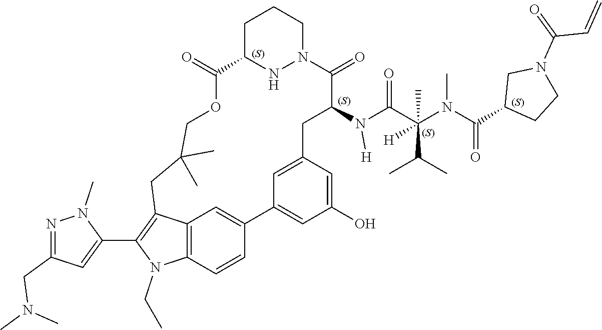 |
| BA110 | 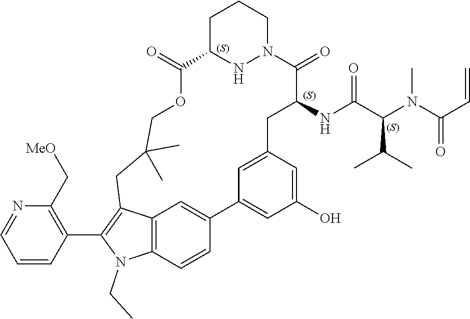 |
| BA111 | 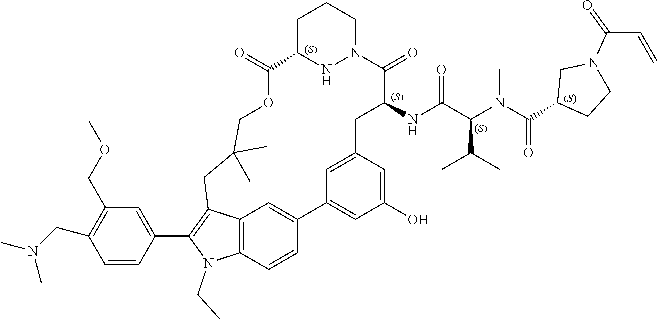 |
| BA112 | 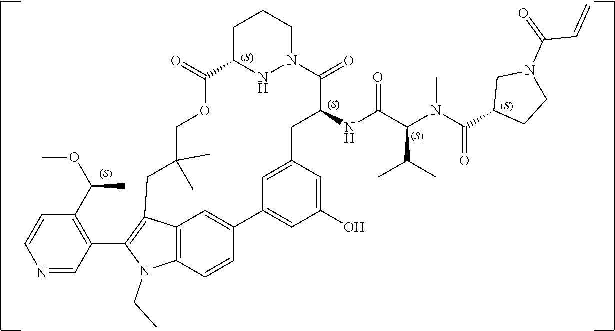 |
| BA113 | 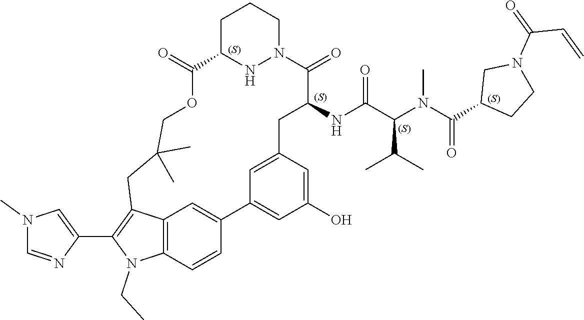 |
| BA114 | 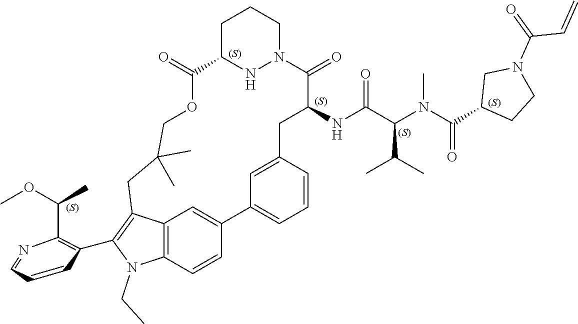 |
| BA115 | 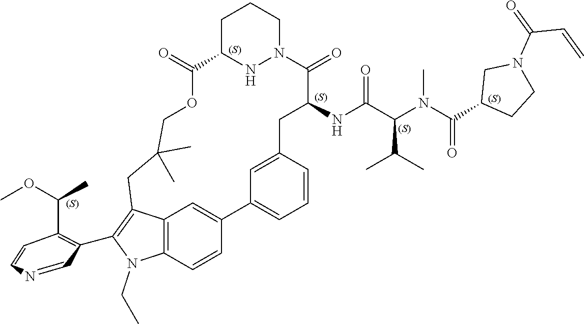 |
| BA116 | 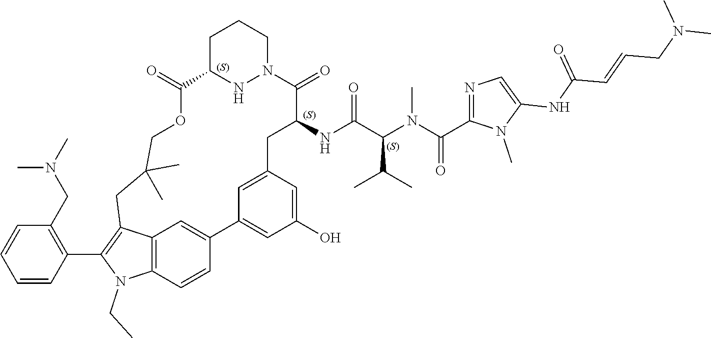 |
| BA117 | 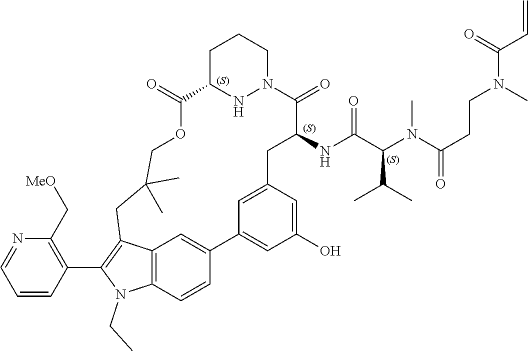 |
| BA118 | 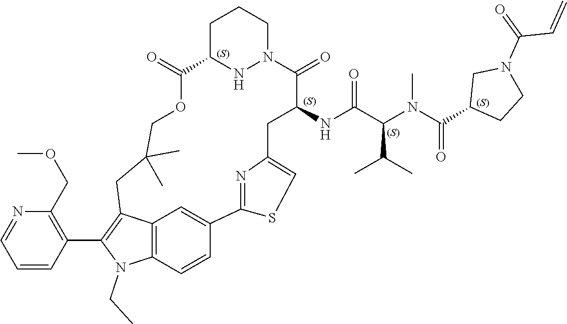 |
| BA119 | 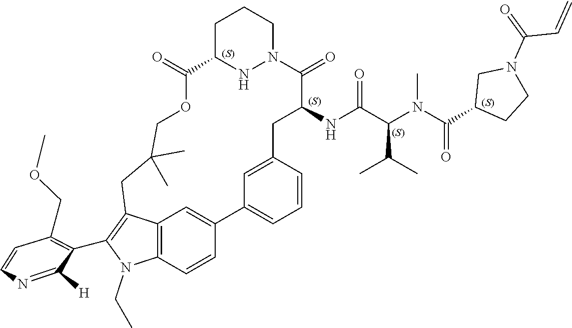 |
| BA120 | 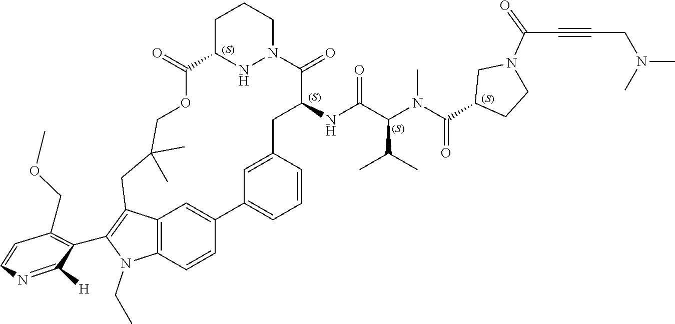 |
| BA121 | 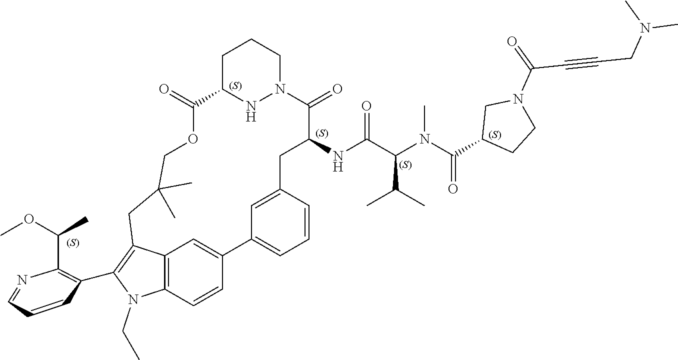 |
| BA122 | 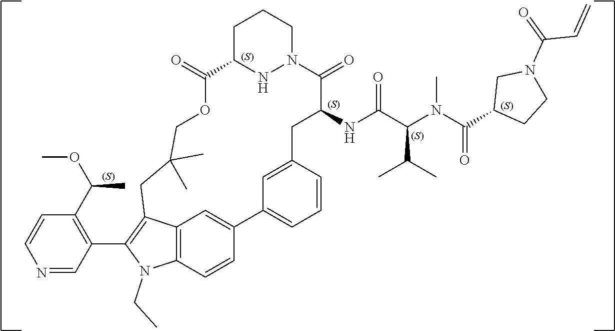 |
| BA123 | 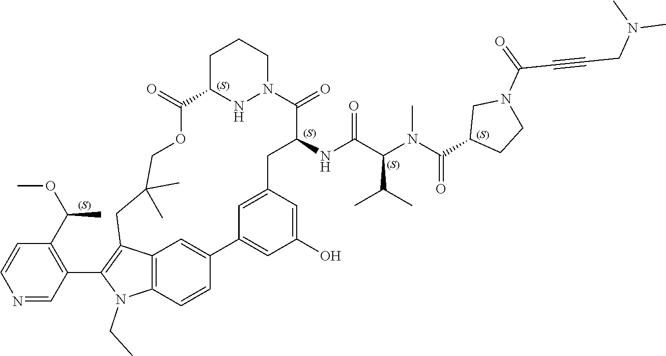 |
| BA124 | 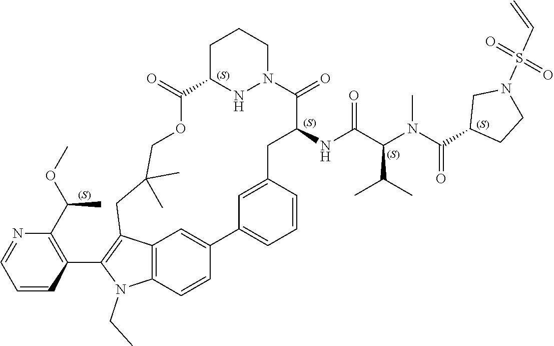 |
| BA125 | 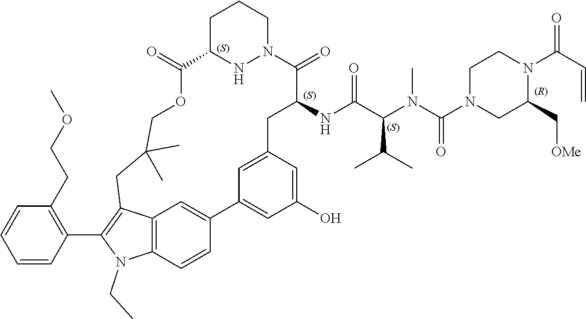 |
| BA126 | 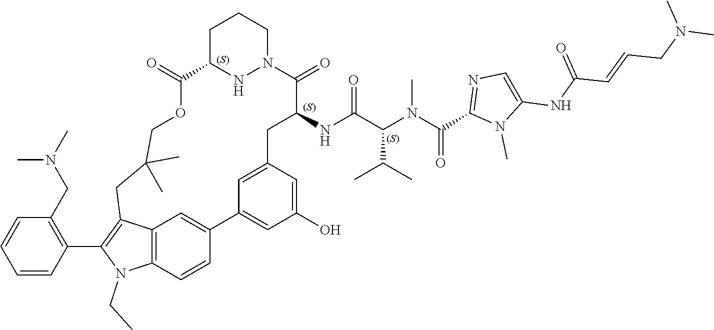 |
| BA127 | 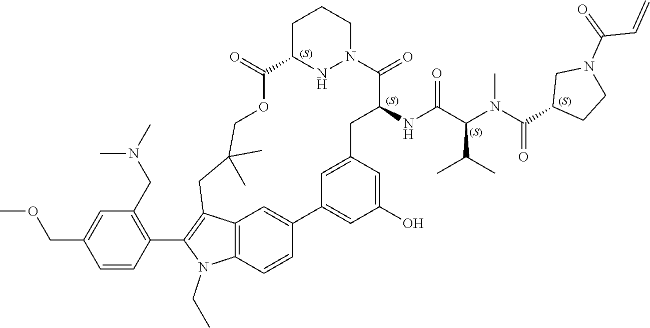 |
| BA128 | 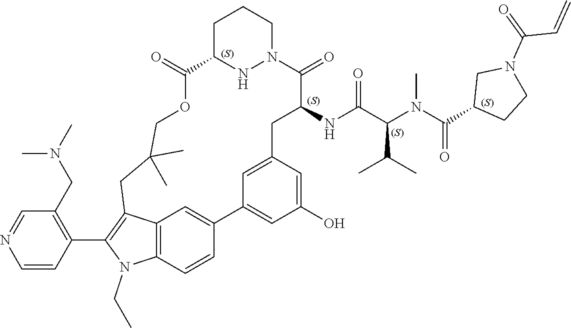 |
| BA129 | 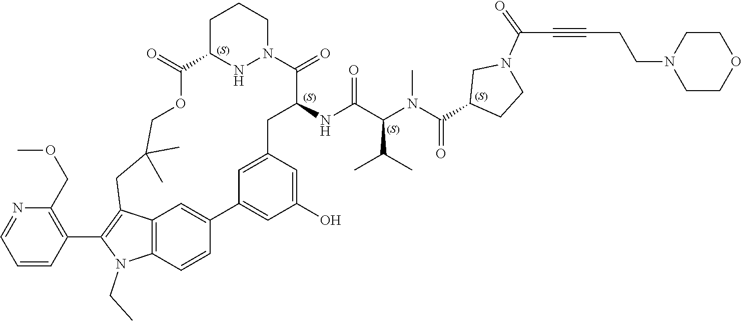 |
| BA130 | 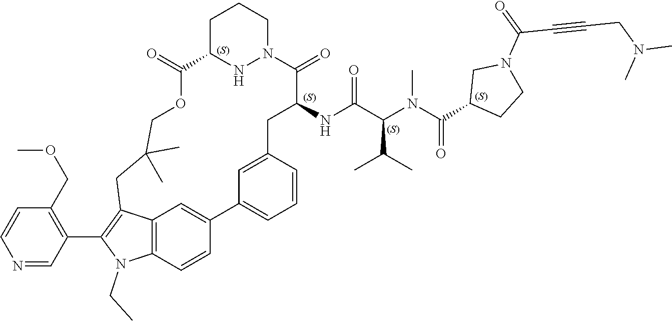 |
| BA131 | 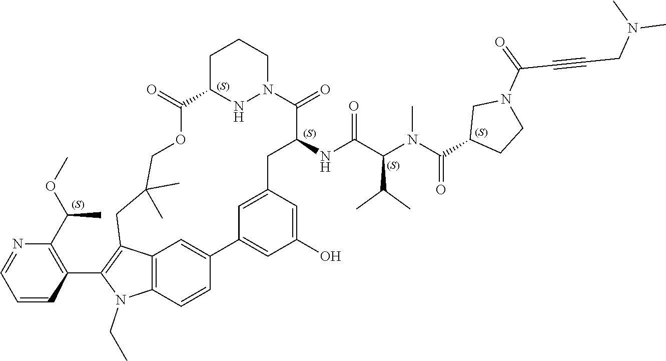 |
| BA132 | 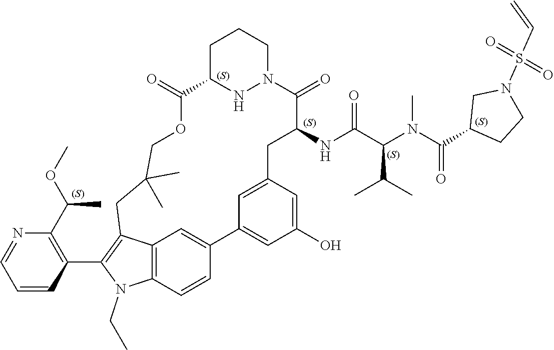 |
| BA133 | 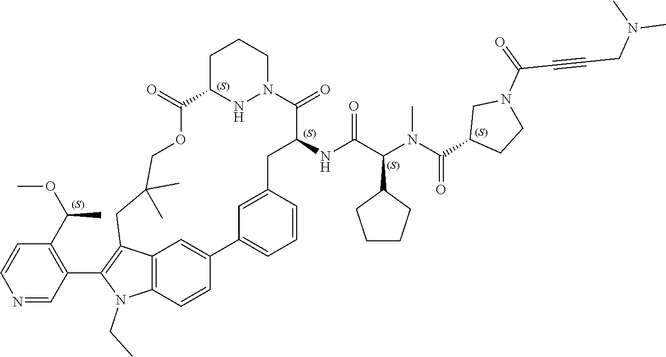 |
| BA134 | 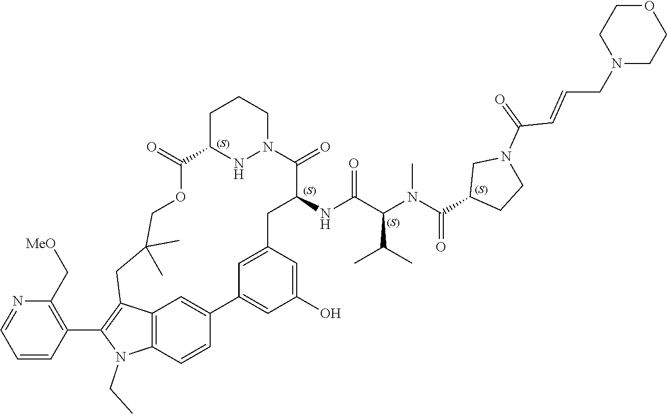 |
| BA135 | 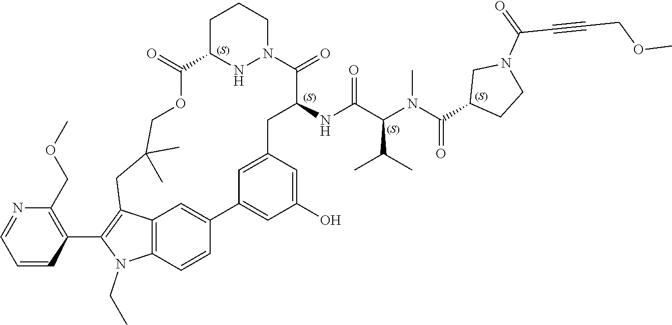 |
| BA136 | 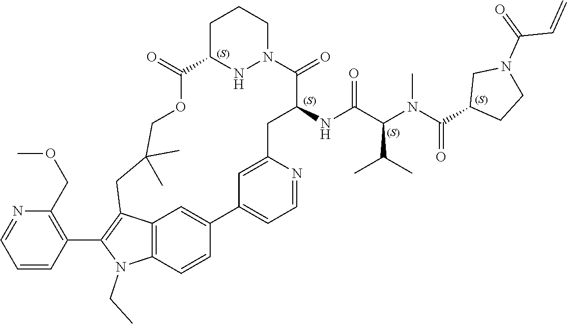 |
| BA137 | 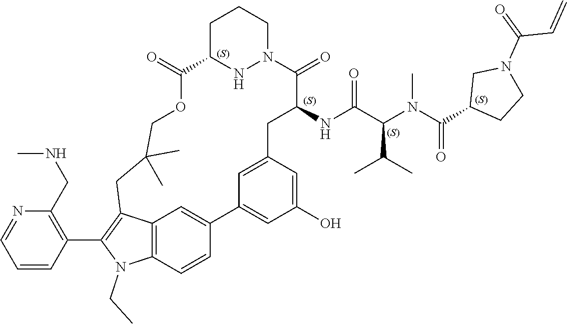 |
| BA138 | 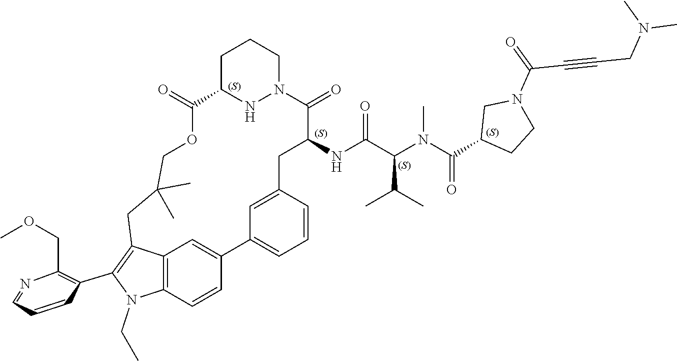 |
| BA139 | 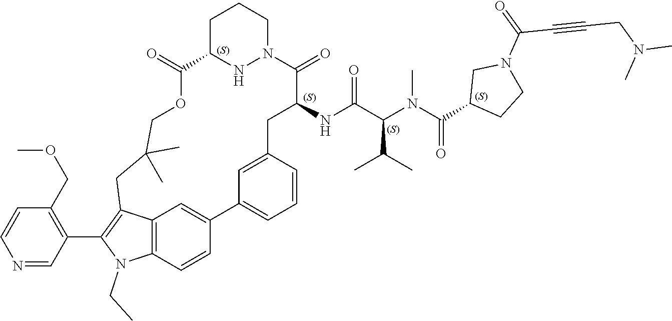 |
| BA140 | 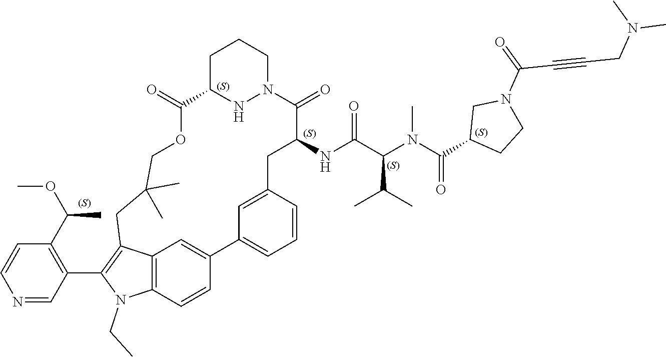 |
| BA141 | 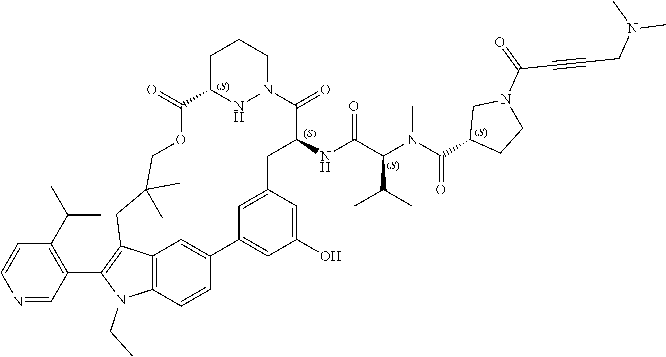 |
| BA142 | 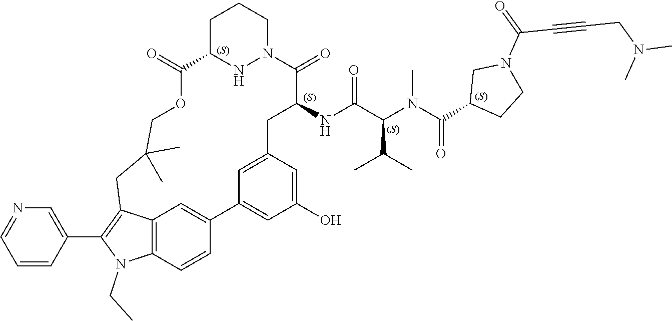 |
| BA143 | 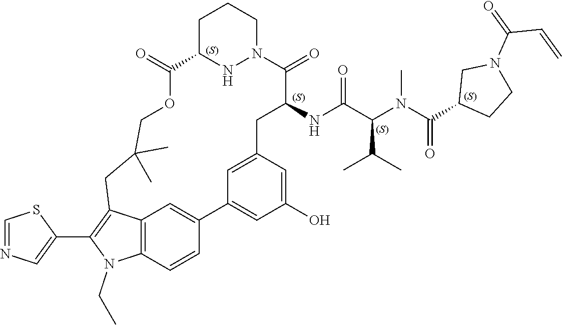 |
| BA144 | 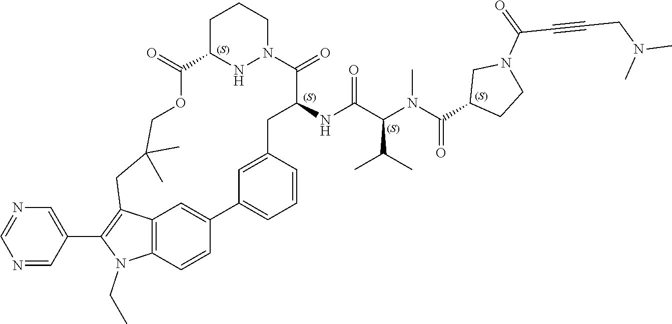 |
| BA145 | 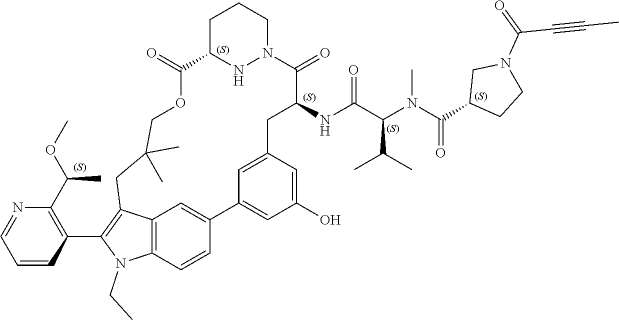 |
| BA146 | 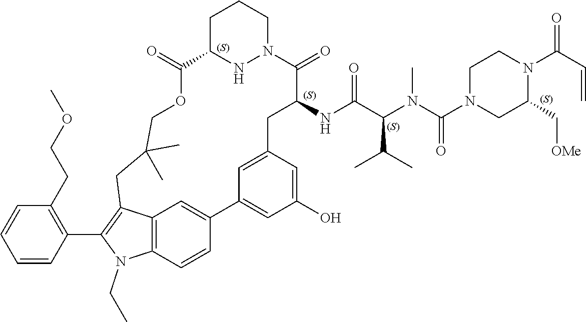 |
| BA147 | 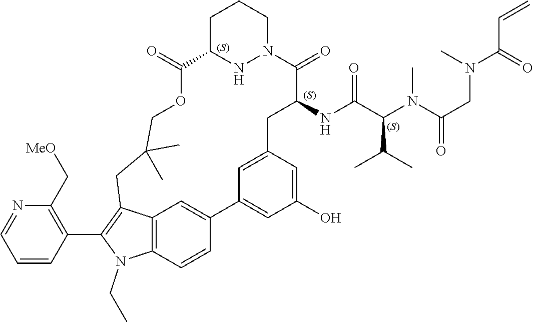 |
| BA148 | 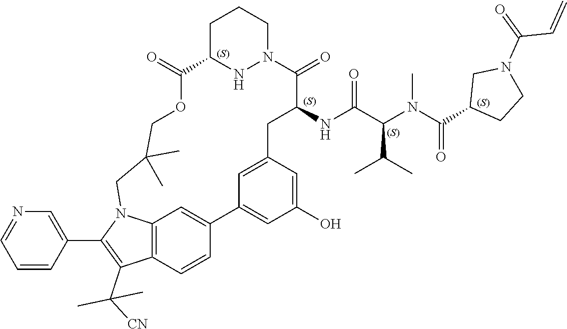 |
| BA149 | 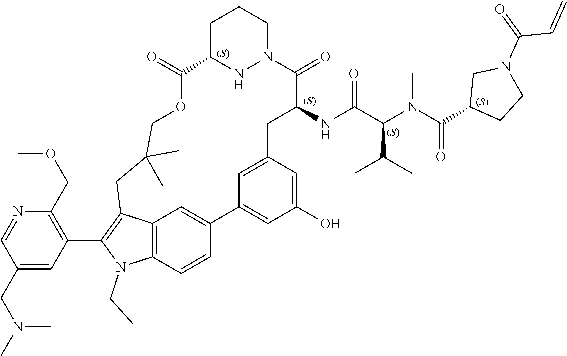 |
| BA150 | 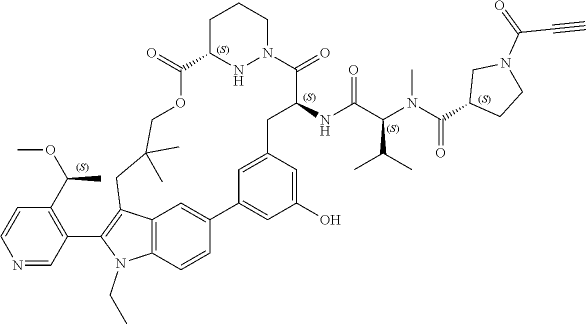 |
| BA151 | 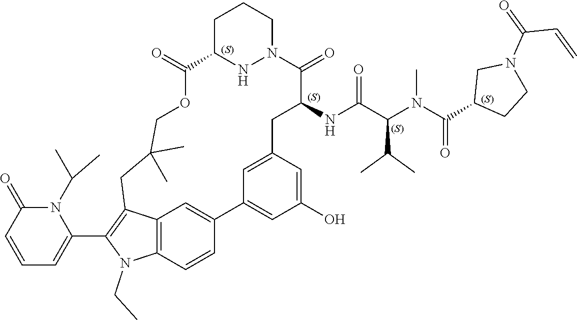 |
| BA152 | 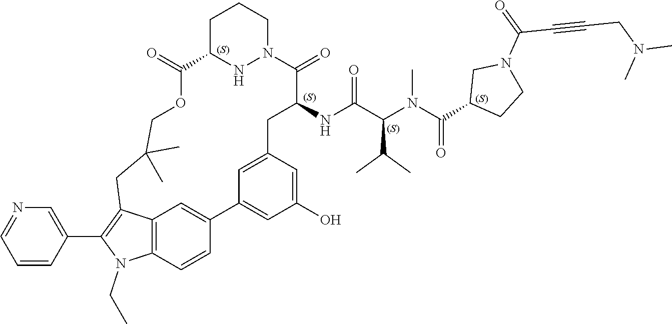 |
| BA153 | 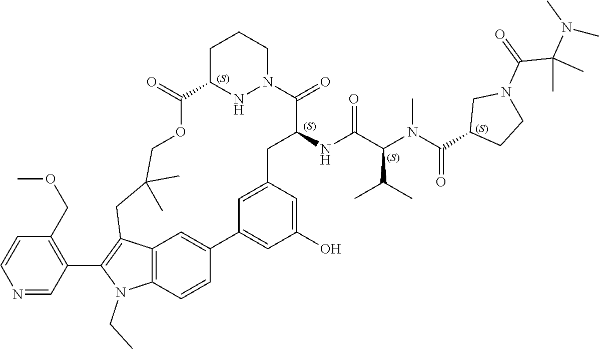 |
| BA154 | 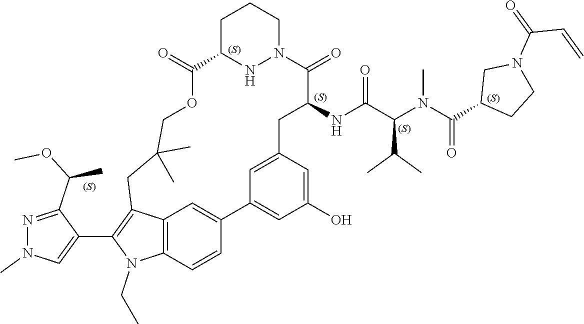 |
| BA155 | 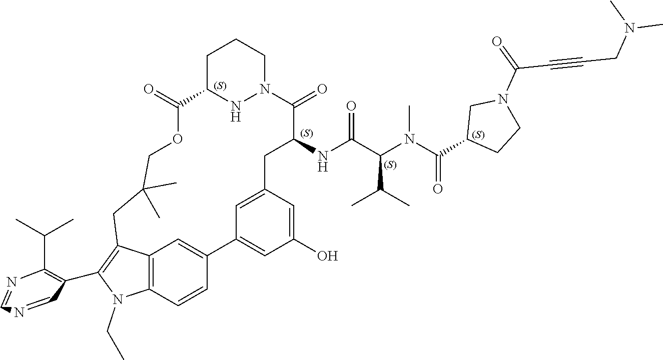 |
| BA156 | 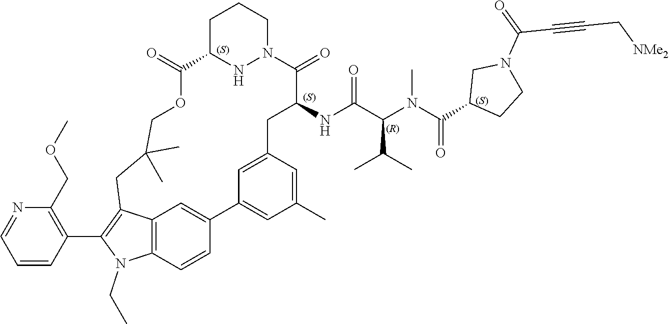 |
| BA157 | 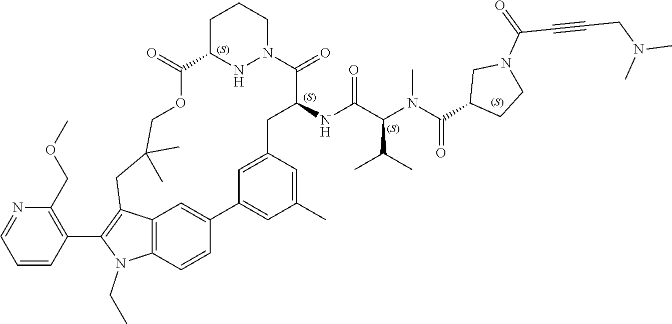 |
| BA158 | 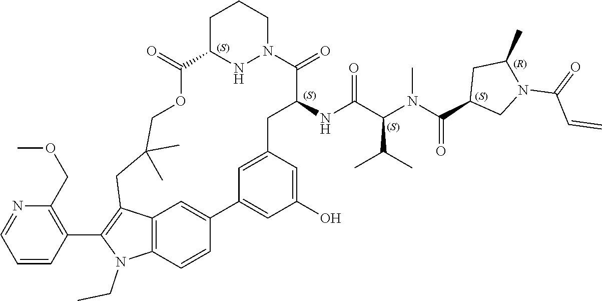 |
| BA159 | 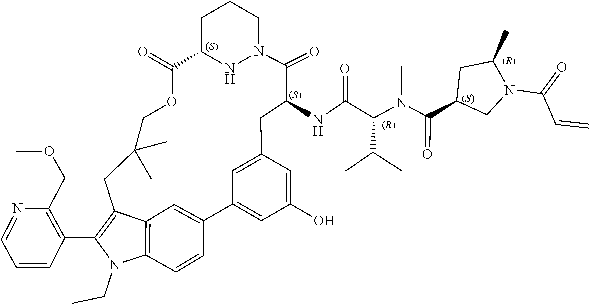 |
| BA160 | 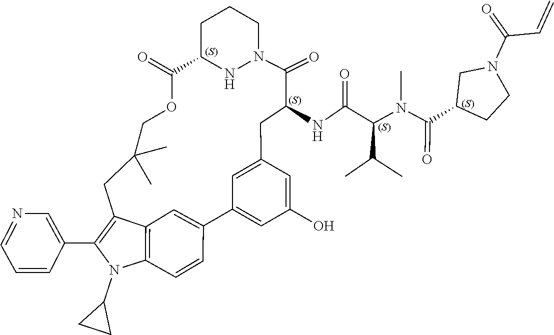 |
| BA161 | 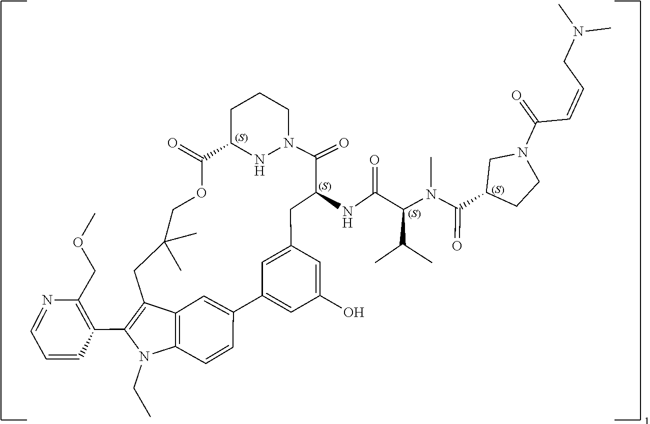 |
| BA162 | 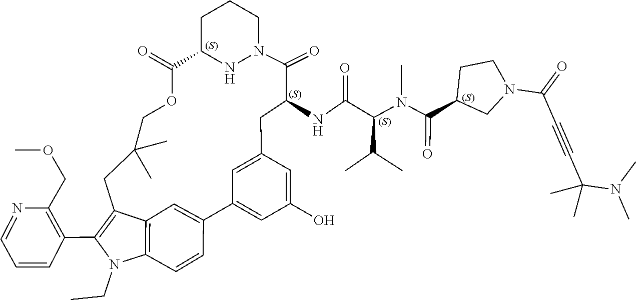 |
| BA163 | 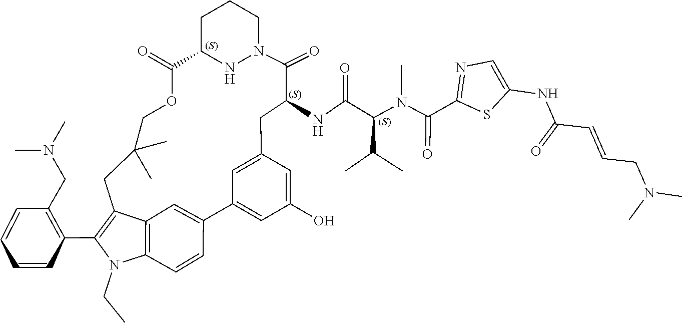 |
| BA164 | 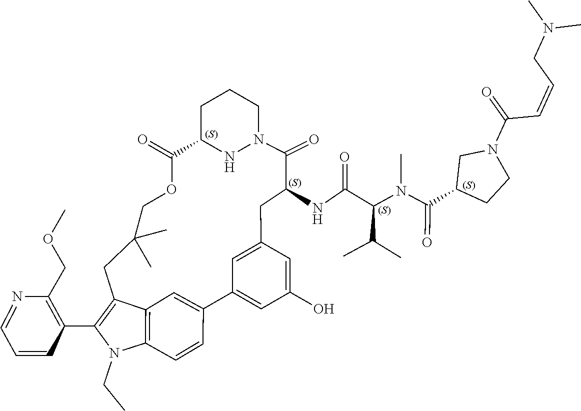 |
| BA165 | 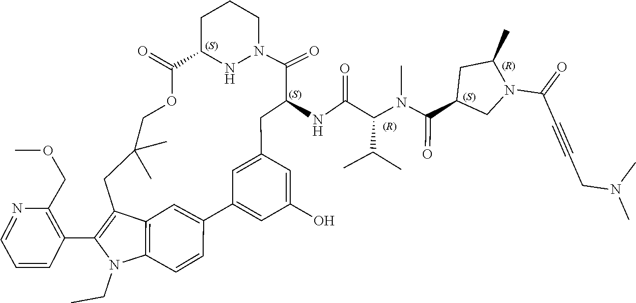 |
| BA166 | 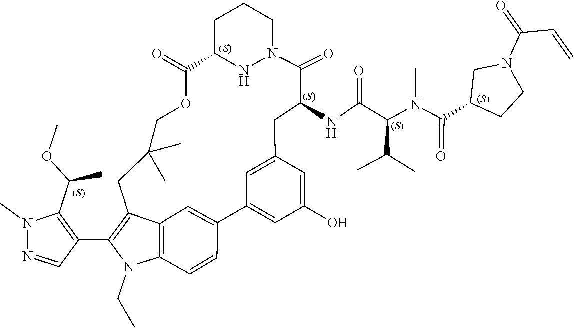 |
| BA167 | 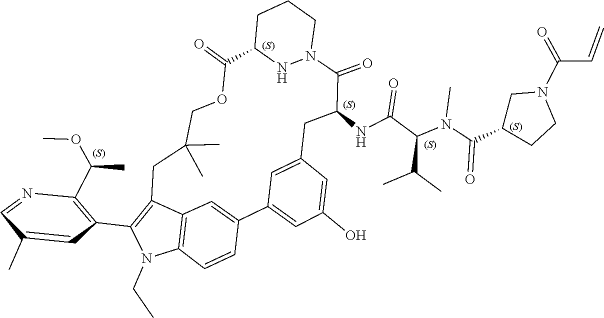 |
| BA168 | 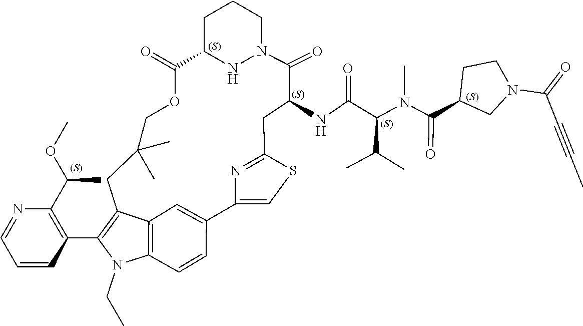 |
| BA169 | 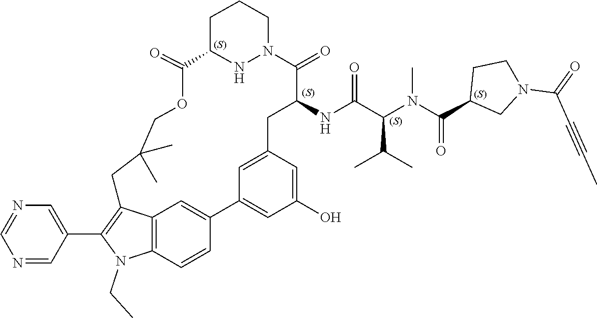 |
| BA170 | 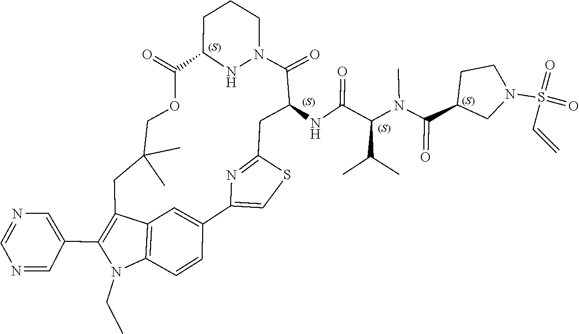 |
| BA171 | 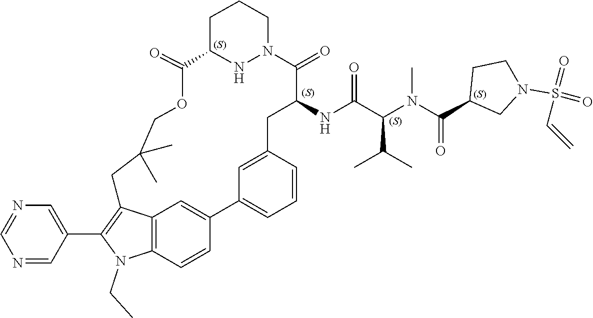 |
| BA172 | 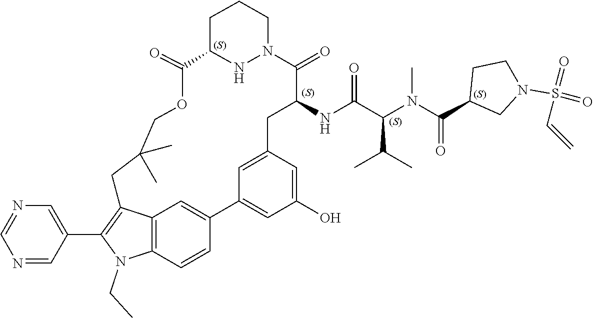 |
| BA173 | 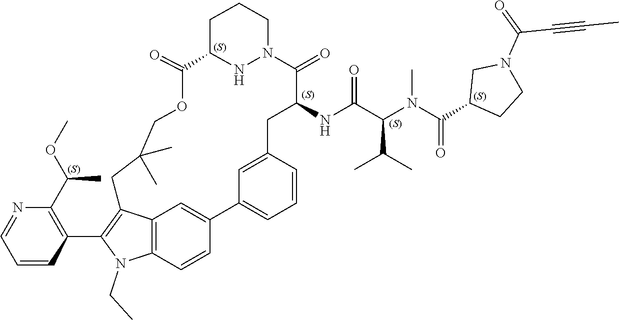 |
| BA174 | 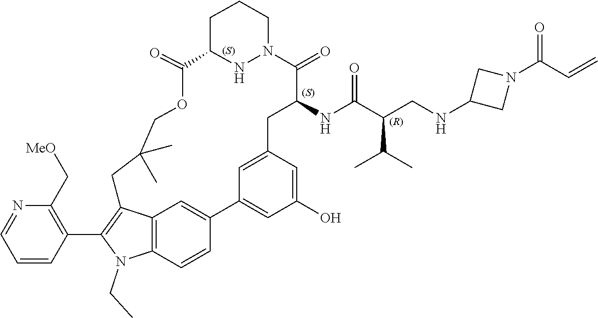 |
| BA175 | 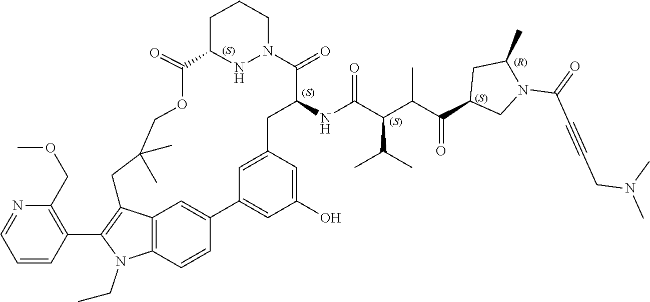 |
| BA176 | 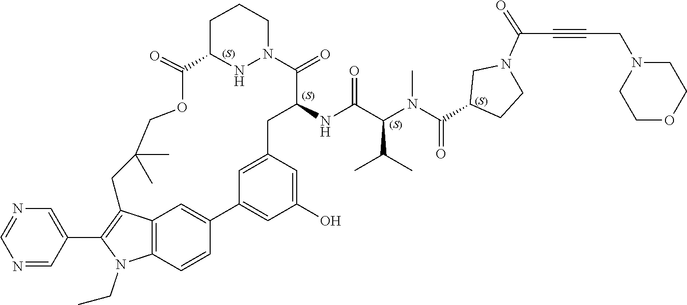 |
| BA177 | 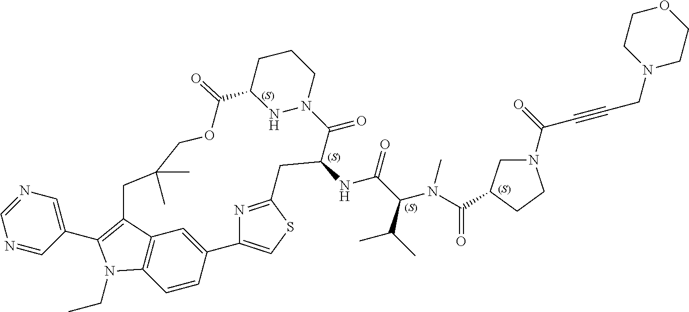 |
| BA178 | 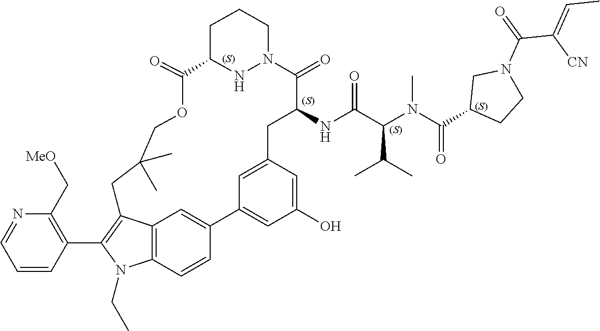 |
| BA179 | 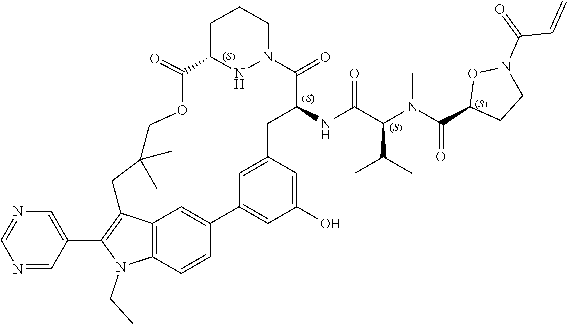 |
| BA180 | 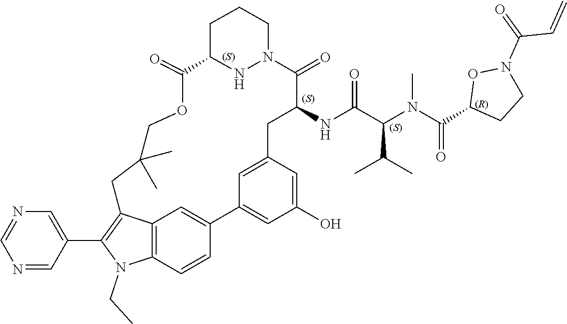 |
| BA181 | 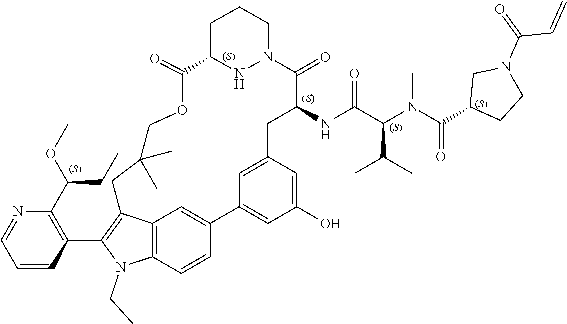 |
| BA182 | 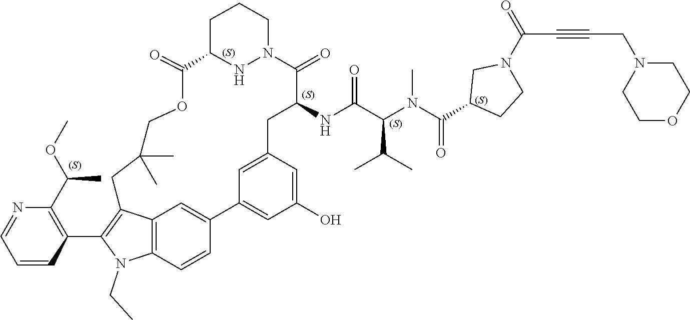 |
| BA183 | 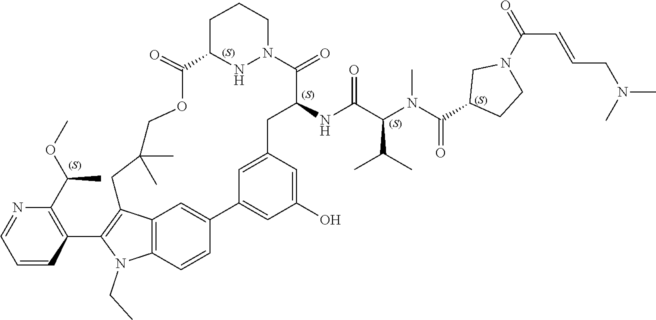 |
| BA184 | 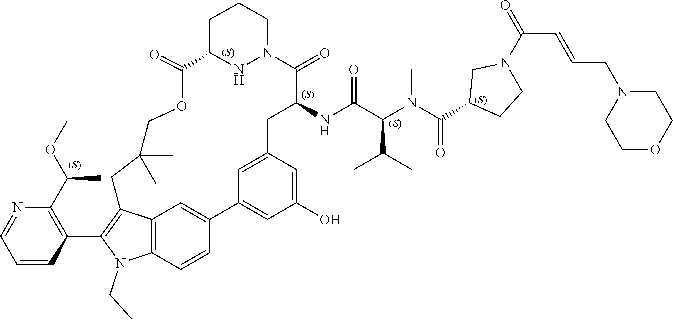 |
| BA185 | 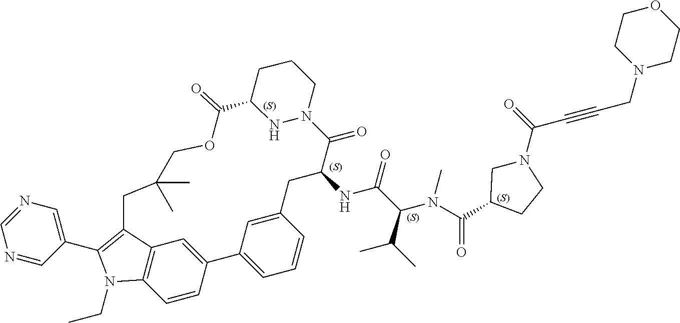 |
| BA186 | 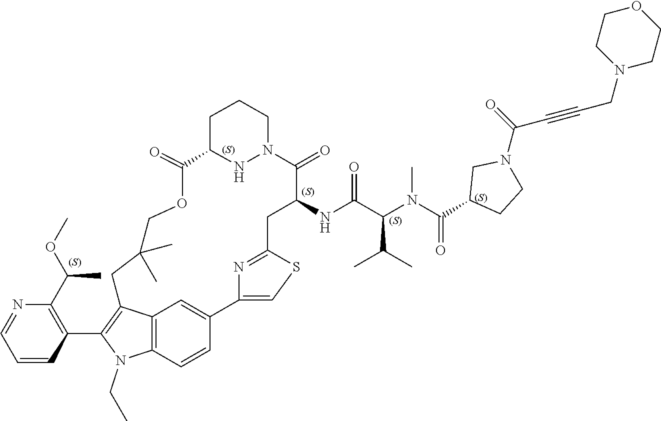 |
| BA187 | 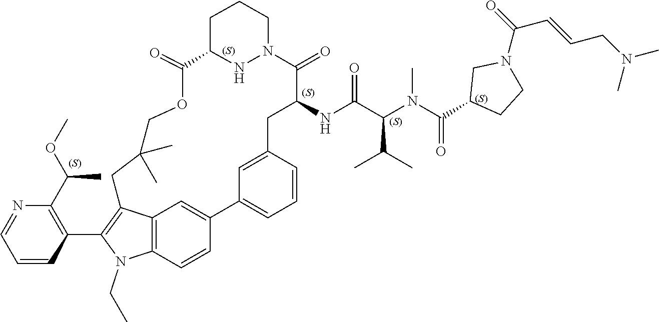 |
| BA188 | 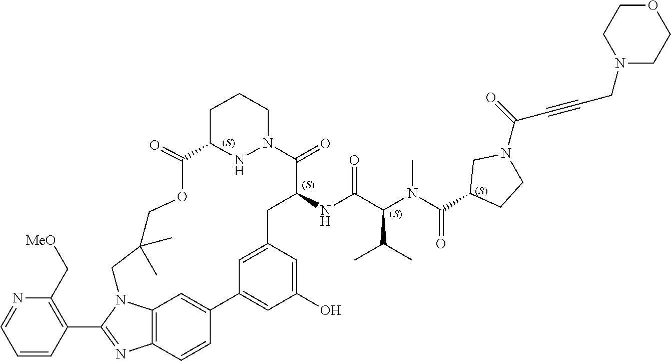 |
| BA189 | 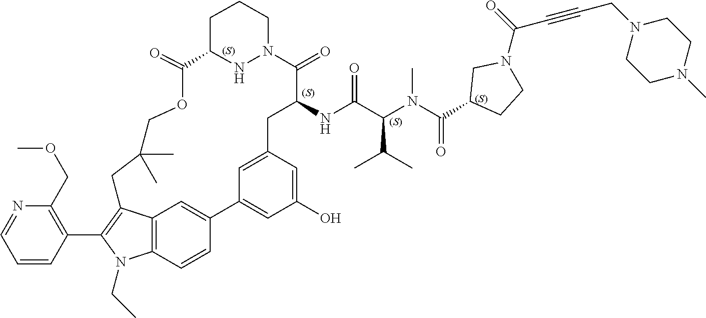 |
| BA190 | 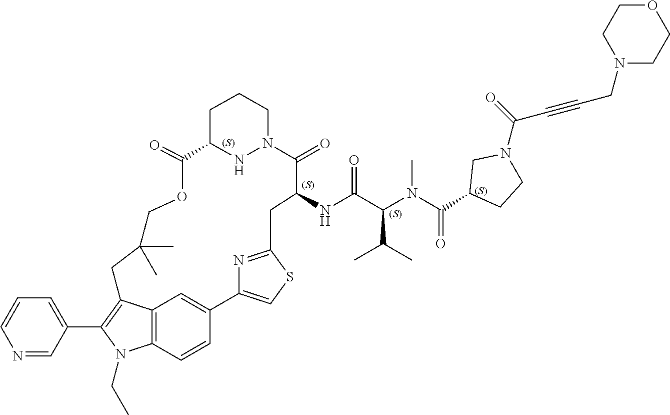 |
| BA191 | 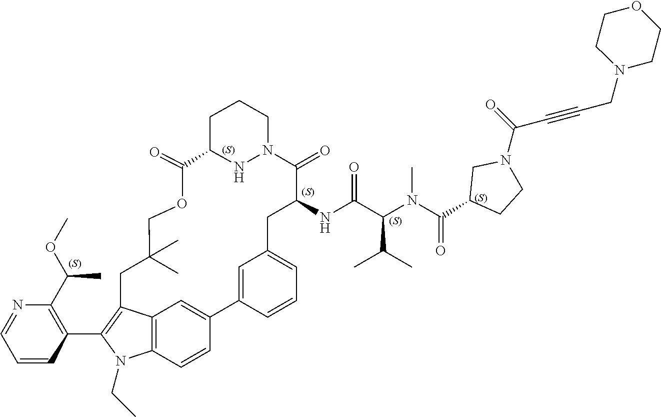 |
| BA192 | 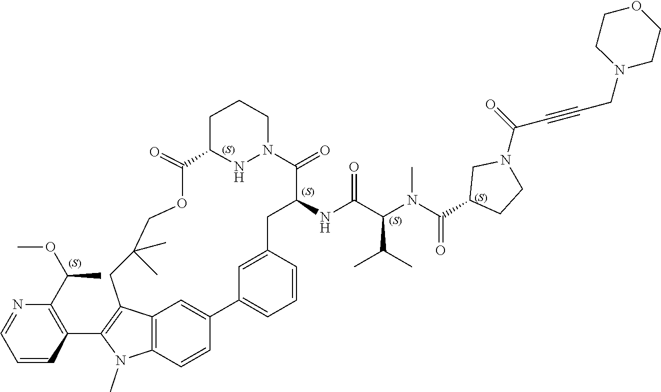 |
| BA193 | 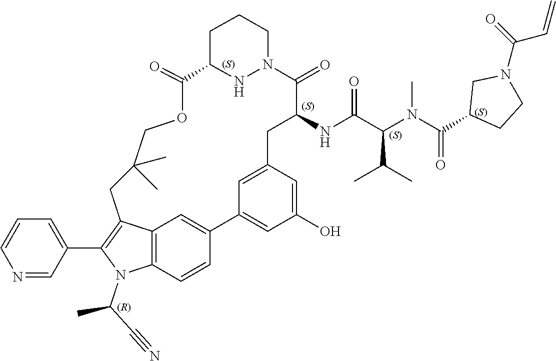 |
| BA194 | 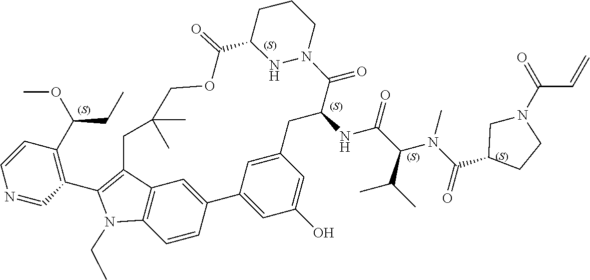 |
| BA195 | 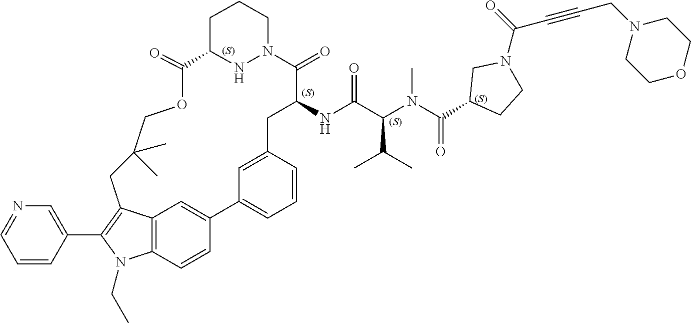 |
| BA196 | 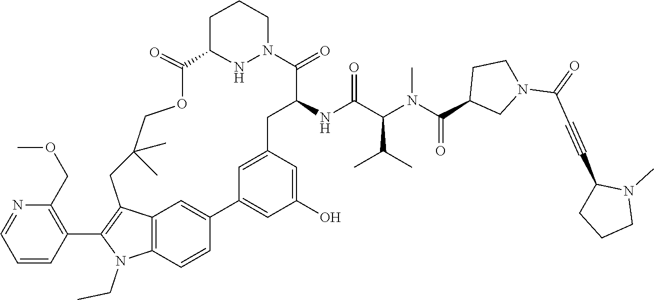 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| BA197 | 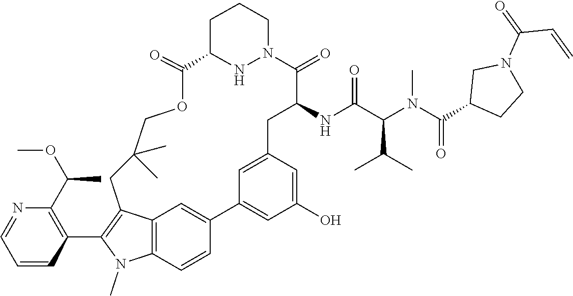 |
| BA198 | 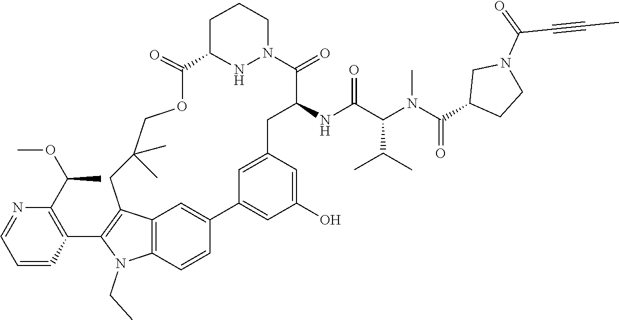 |
| BA199 | 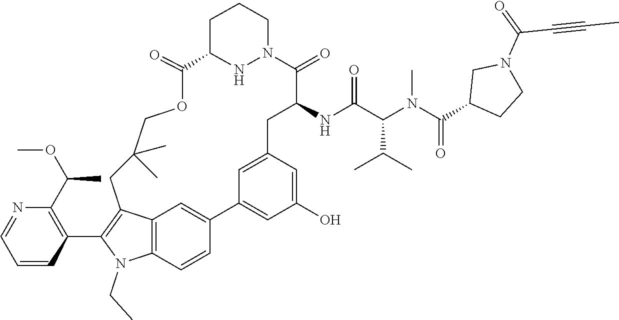 |
| BA200 | 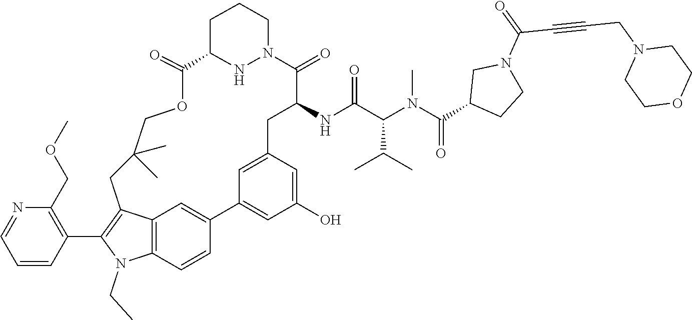 [SMILES: CCn1c(-c2cccnc2COC)c2c3cc(ccc31)-c1cc(O)cc(c1)C[C@H](NC(=O)[C@@H](C(C)C)N(C)C(=O)[C@H]1CCN(C(=O)C#CCN3CCOCC3)C1)C(=O)N1CCC[C@H](N1)C(=O)OCC(C)(C)C2] |
| BA201 | 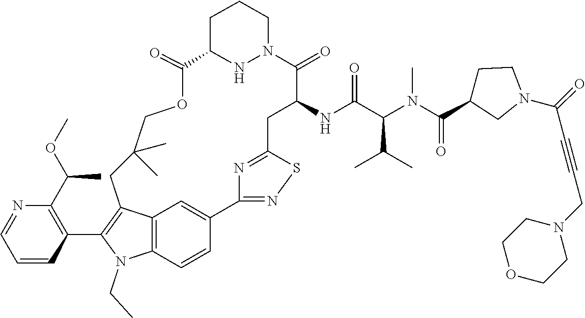 |
| BA202 | 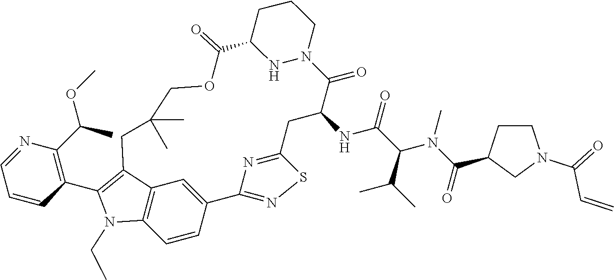 |
| BA203 | 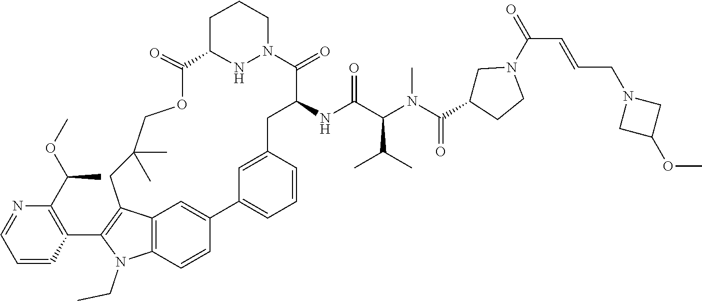 |
| BA204 | 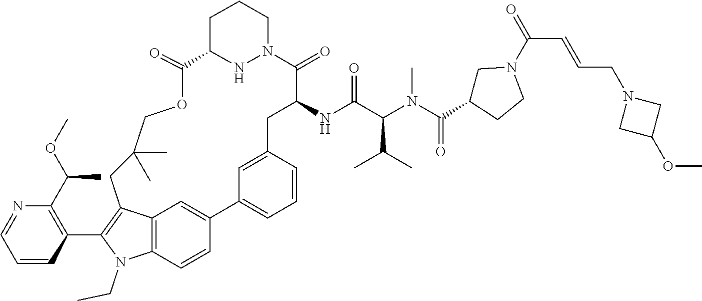 |
| BA205 | 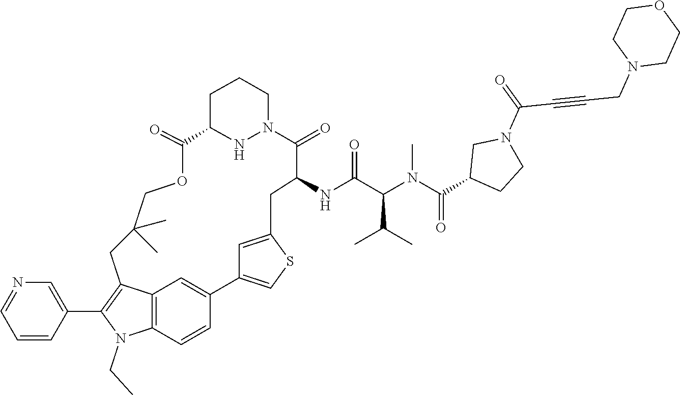 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BA206 | 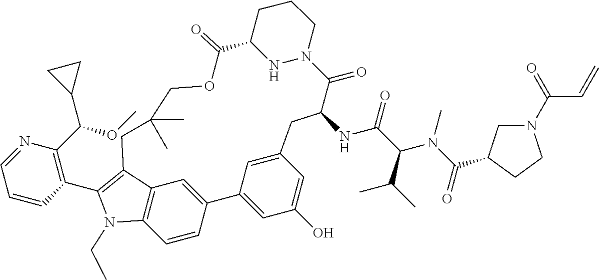 |
| BA207 | 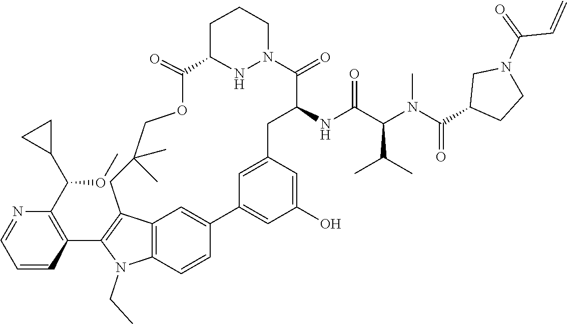 |
| BA208 | 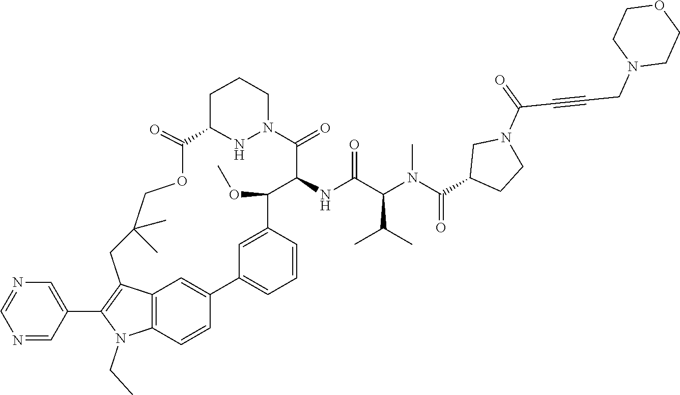 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BA209 | 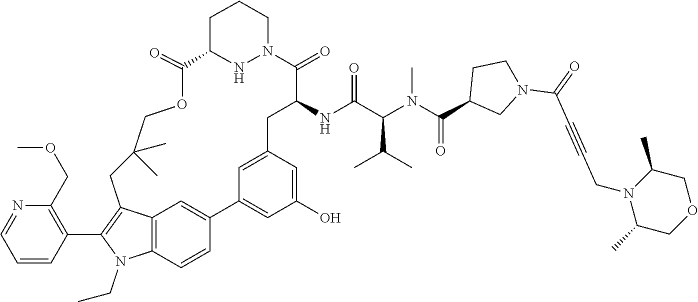 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@@H](C)COC[C@@H]3C)C1)C(=O)N1CCC[C@H](N1)C(=O)OCC(C)(C)C2] |
| BA210 | 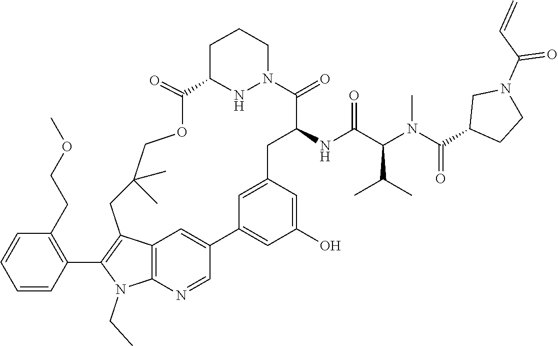 [SMILES: C=CC(=O)N1CC[C@H](C(=O)N(C)[C@H](C(=O)N[C@H]2Cc3cc(O)cc(c3)-c3cnc4c(c3)c(c(-c3ccccc3CCOC)n4CC)CC(C)(C)COC(=O)[C@@H]3CCCN(N3)C2=O)C(C)C)C1] |
| BA211 | 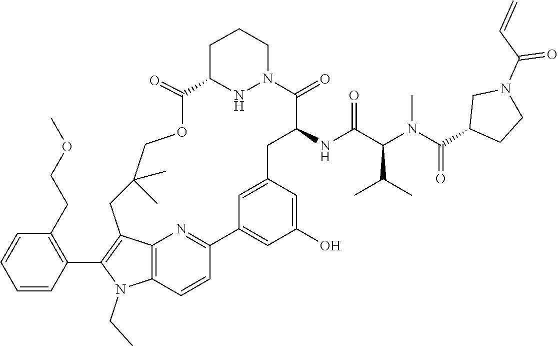 [SMILES: C=CC(=O)N1CC[C@H](C(=O)N(C)[C@H](C(=O)N[C@H]2Cc3cc(O)cc(c3)-c3ccc4c(n3)c(c(-c3ccccc3CCOC)n4CC)CC(C)(C)COC(=O)[C@@H]3CCCN(N3)C2=O)C(C)C)C1] |
| BA212 | 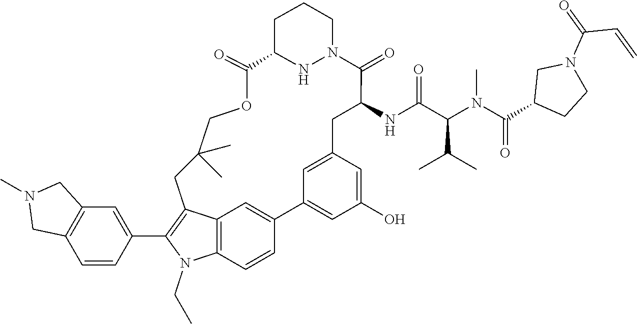 [SMILES: C=CC(=O)N1CC[C@H](C(=O)N(C)[C@H](C(=O)N[C@H]2Cc3cc(O)cc(c3)-c3ccc4c(c3)c(c(-c3ccc5c(c3)CN(C)C5)n4CC)CC(C)(C)COC(=O)[C@@H]3CCCN(N3)C2=O)C(C)C)C1] |
| BA213 | 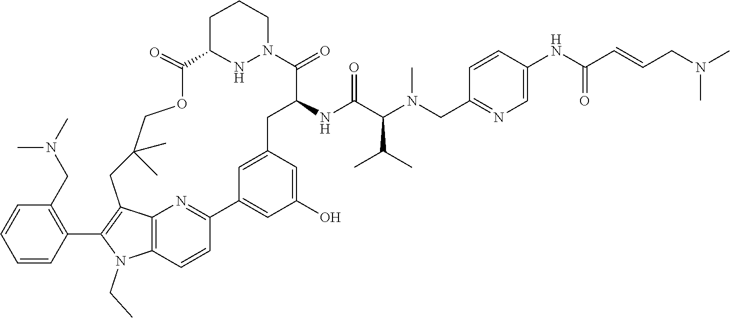 [SMILES: CCn1c(-c2ccccc2CN(C)C)c2c3nc(ccc31)-c1cc(O)cc(c1)C[C@H](NC(=O)[C@H](C(C)C)N(C)Cc1ccc(NC(=O)/C=C/CN(C)C)cn1)C(=O)N1CCC[C@H](N1)C(=O)OCC(C)(C)C2] |
| BA214 | 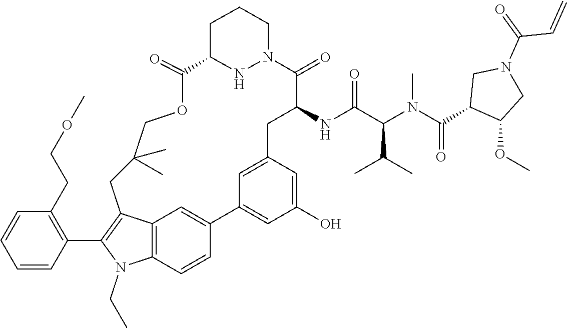 [SMILES: C=CC(=O)N1C[C@H](OC)[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| BA215 | 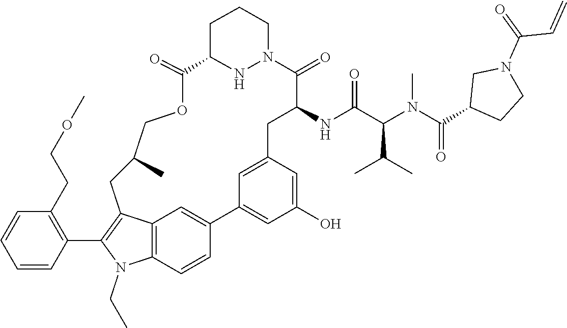 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)C[C@H](C)COC(=O)[C@@H]3CCCN(N3)C2=O)C(C)C)C1] |
| BA216 | 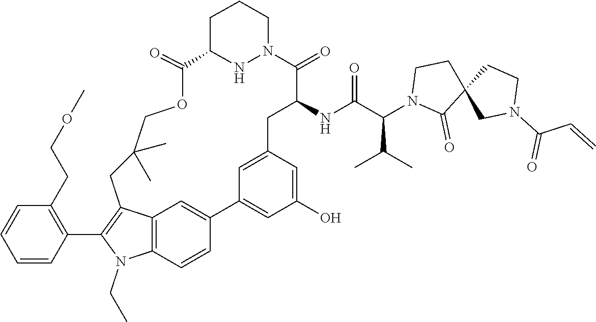 [SMILES: C=CC(=O)N1CC[C@]2(CCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| BA217 | 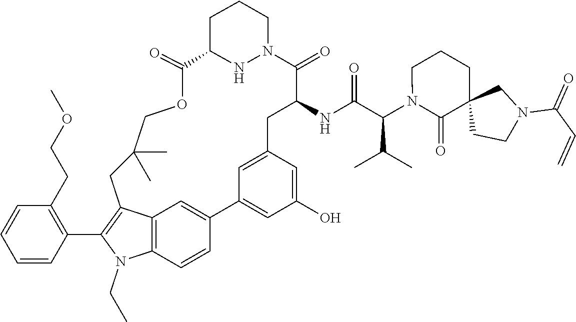 [SMILES: C=CC(=O)N1CC[C@]2(CCCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| BA218 | 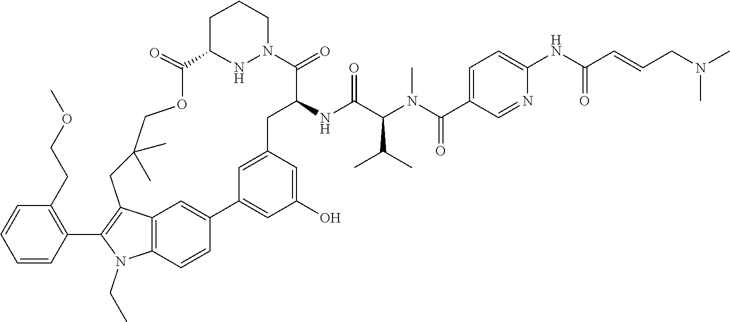 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)nc1)C(=O)N1CCC[C@H](N1)C(=O)OCC(C)(C)C2] |
| BA219 | 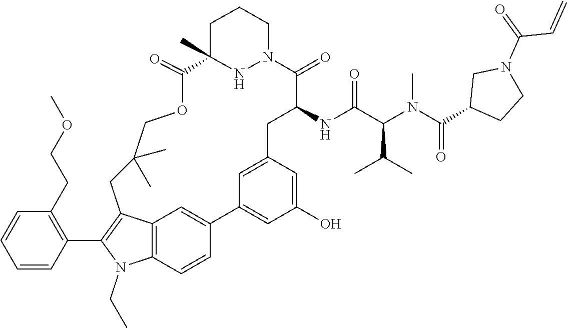 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@]3(C)CCCN(N3)C2=O)C(C)C)C1] |
| BA220 | 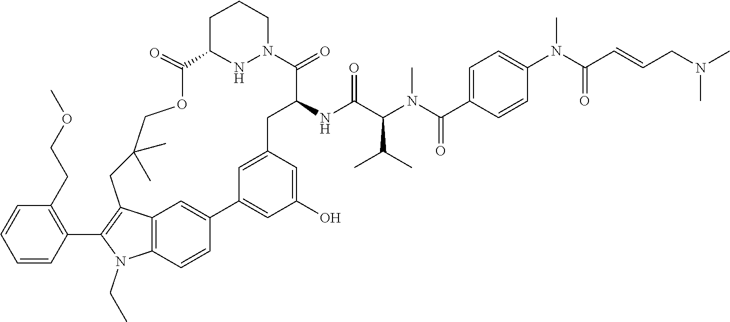 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(N(C)C(=O)/C=C/CN(C)C)cc1)C(=O)N1CCC[C@H](N1)C(=O)OCC(C)(C)C2] |
| BA221 | 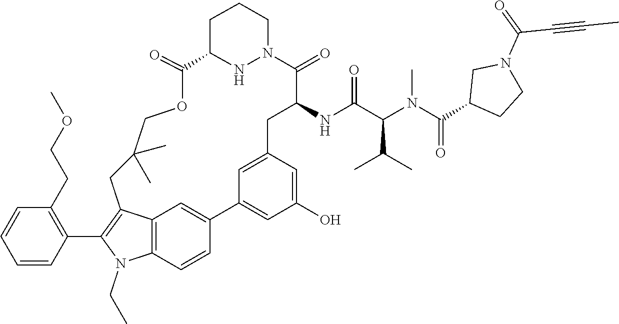 [SMILES: CC#CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| BA222 | 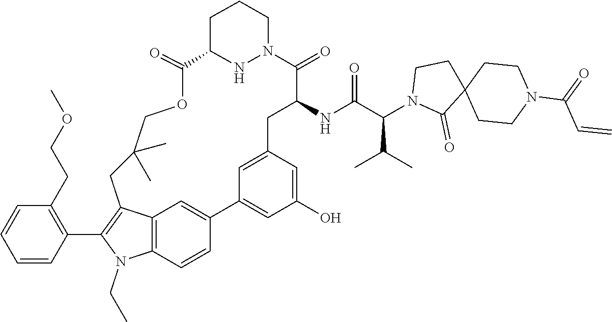 [SMILES: C=CC(=O)N1CCC2(CC1)CCN([C@H](C(=O)N[C@H]1Cc3cc(O)cc(c3)-c3ccc4c(c3)c(c(-c3ccccc3CCOC)n4CC)CC(C)(C)COC(=O)[C@@H]3CCCN(N3)C1=O)C(C)C)C2=O] |
| BA223 | 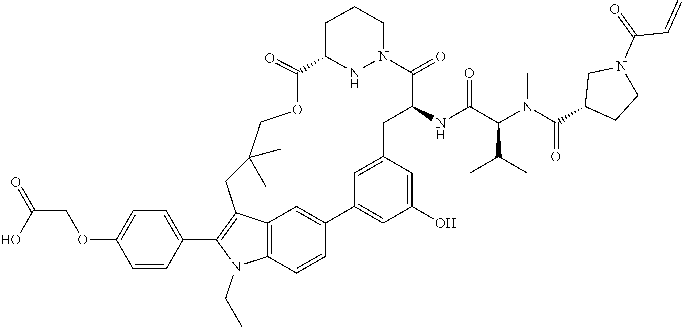 [SMILES: C=CC(=O)N1CC[C@H](C(=O)N(C)[C@H](C(=O)N[C@H]2Cc3cc(O)cc(c3)-c3ccc4c(c3)c(c(-c3ccc(OCC(=O)O)cc3)n4CC)CC(C)(C)COC(=O)[C@@H]3CCCN(N3)C2=O)C(C)C)C1] |
| BA224 | 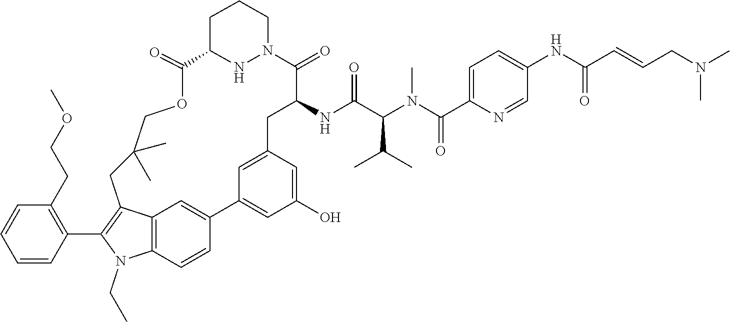 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| BA225 | 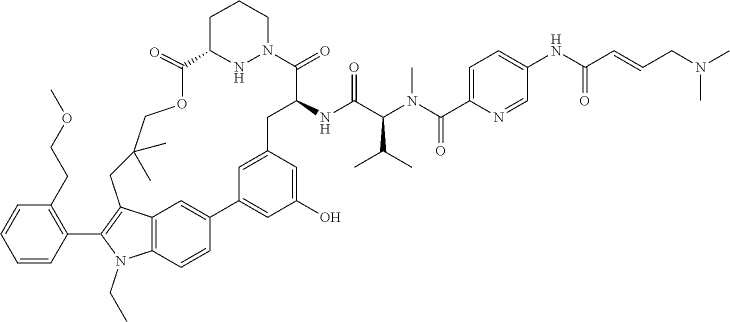 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| BA226 | 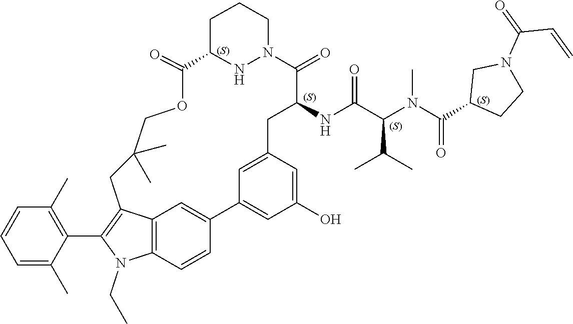 |
| BA227 | 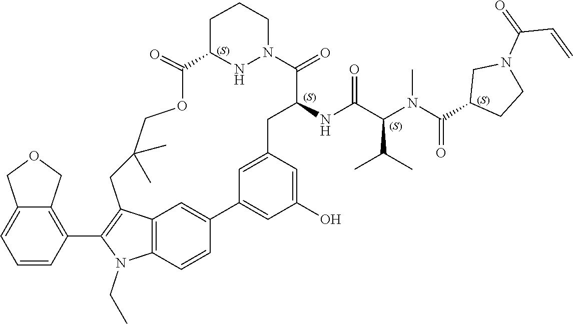 |
| BA228 | 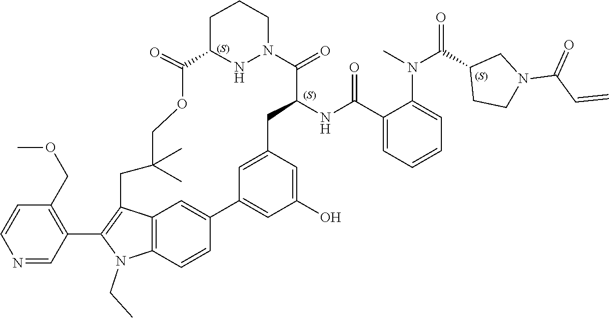 |
| BA229 | 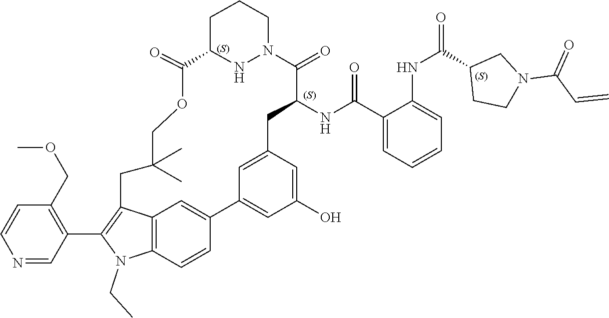 |
| BA230 | 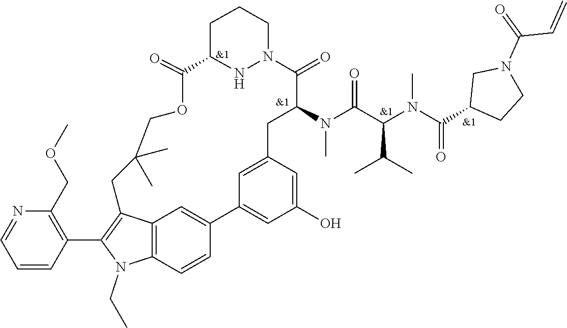 [SMILES: C=CC(=O)N1CC[C@H](C(=O)N(C)[C@H](C(=O)N(C)[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| BA231 | 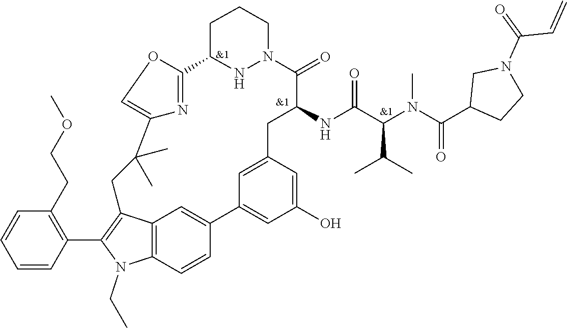 [SMILES: C=CC(=O)N1CCC(C(=O)N(C)[C@H](C(=O)N[C@H]2Cc3cc(O)cc(c3)-c3ccc4c(c3)c(c(-c3ccccc3CCOC)n4CC)CC(C)(C)c3coc(n3)[C@@H]3CCCN(N3)C2=O)C(C)C)C1] |
| BA232 | 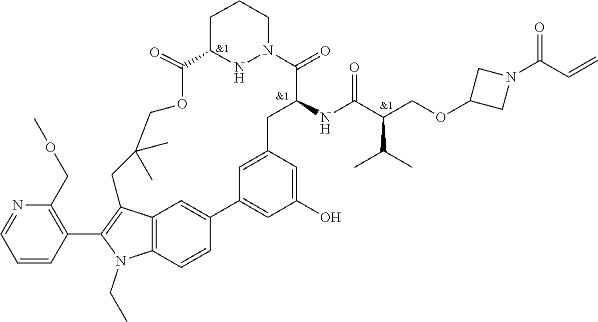 [SMILES: C=CC(=O)N1CC(OC[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| BA233 | 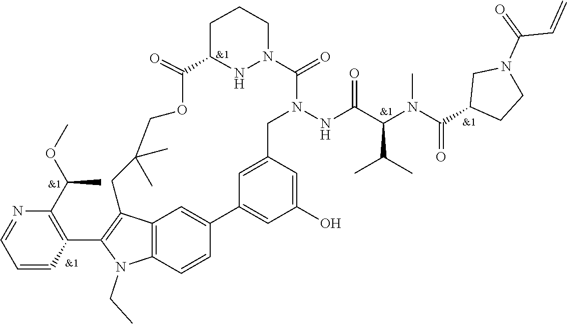 |
| BA234 | 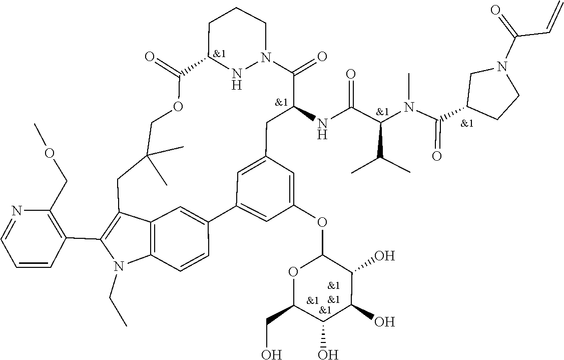 [SMILES: C=CC(=O)N1CC[C@H](C(=O)N(C)[C@H](C(=O)N[C@H]2Cc3cc(OC4O[C@H](CO)[C@@H](O)[C@H](O)[C@H]4O)cc(c3)-c3ccc4c(c3)c(c(-c3cccnc3COC)n4CC)CC(C)(C)COC(=O)[C@@H]3CCCN(N3)C2=O)C(C)C)C1] |
| BA235 | 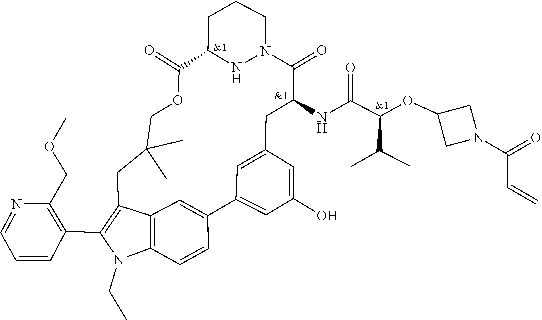 [SMILES: C=CC(=O)N1CC(O[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| BA236 | 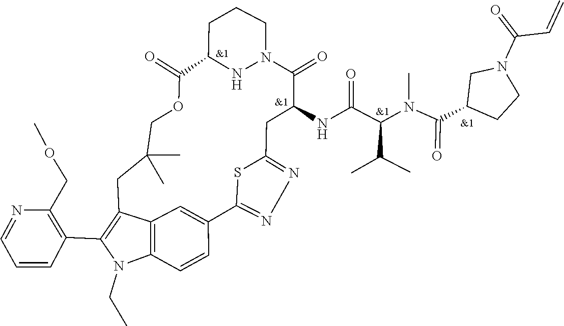 [SMILES: C=CC(=O)N1CC[C@H](C(=O)N(C)[C@H](C(=O)N[C@H]2Cc3nnc(s3)-c3ccc4c(c3)c(c(-c3cccnc3COC)n4CC)CC(C)(C)COC(=O)[C@@H]3CCCN(N3)C2=O)C(C)C)C1] |
| BA237 | 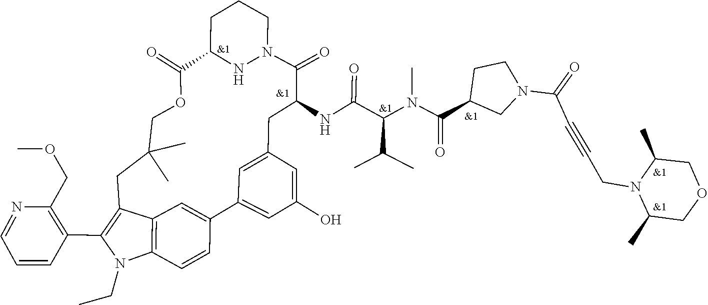 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@H](C)COC[C@@H]3C)C1)C(=O)N1CCC[C@H](N1)C(=O)OCC(C)(C)C2] |
| BA238 | 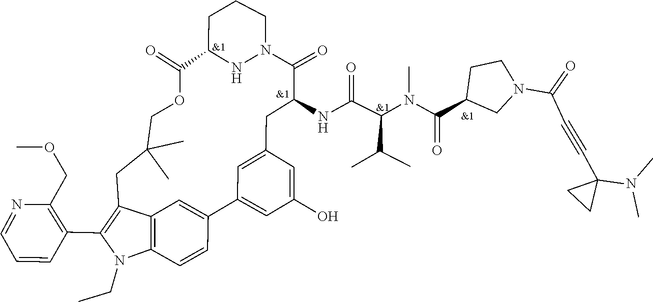 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CC3(N(C)C)CC3)C1)C(=O)N1CCC[C@H](N1)C(=O)OCC(C)(C)C2] |
| BA239 | 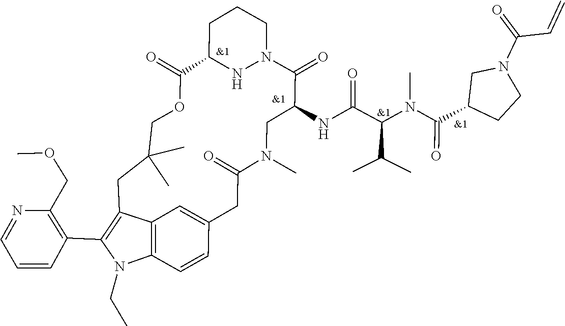 [SMILES: C=CC(=O)N1CC[C@H](C(=O)N(C)[C@H](C(=O)N[C@H]2CN(C)C(=O)Cc3ccc4c(c3)c(c(-c3cccnc3COC)n4CC)CC(C)(C)COC(=O)[C@@H]3CCCN(N3)C2=O)C(C)C)C1] |
| BA240 | 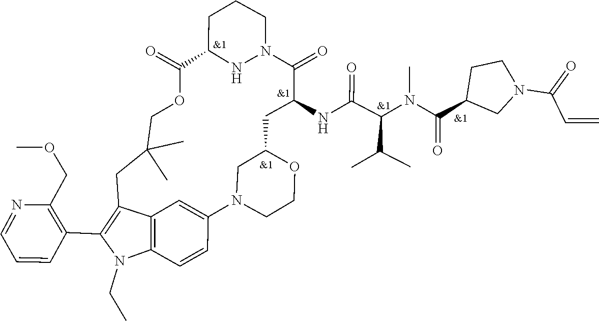 [SMILES: C=CC(=O)N1CC[C@H](C(=O)N(C)[C@H](C(=O)N[C@H]2C[C@H]3CN(CCO3)c3ccc4c(c3)c(c(-c3cccnc3COC)n4CC)CC(C)(C)COC(=O)[C@@H]3CCCN(N3)C2=O)C(C)C)C1] |
| BA241 | 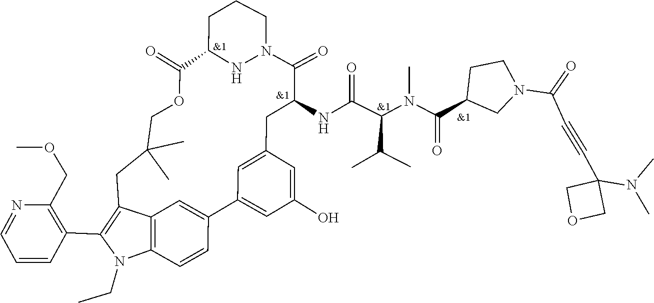 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CC3(N(C)C)COC3)C1)C(=O)N1CCC[C@H](N1)C(=O)OCC(C)(C)C2] |
| BA242 | 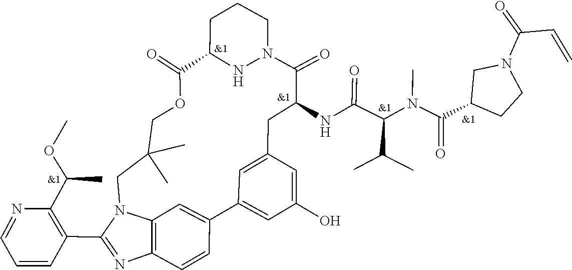 [SMILES: C=CC(=O)N1CC[C@H](C(=O)N(C)[C@H](C(=O)N[C@H]2Cc3cc(O)cc(c3)-c3ccc4nc(-c5cccnc5[C@H](C)OC)n(c4c3)CC(C)(C)COC(=O)[C@@H]3CCCN(N3)C2=O)C(C)C)C1] |
| BA243 | 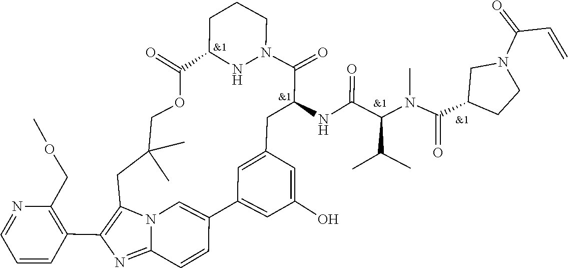 [SMILES: C=CC(=O)N1CC[C@H](C(=O)N(C)[C@H](C(=O)N[C@H]2Cc3cc(O)cc(c3)-c3ccc4nc(-c5cccnc5COC)c(n4c3)CC(C)(C)COC(=O)[C@@H]3CCCN(N3)C2=O)C(C)C)C1] |
| BA244 | 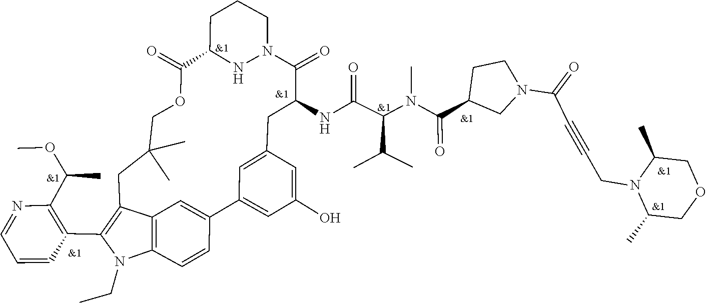 |
| BA245 | 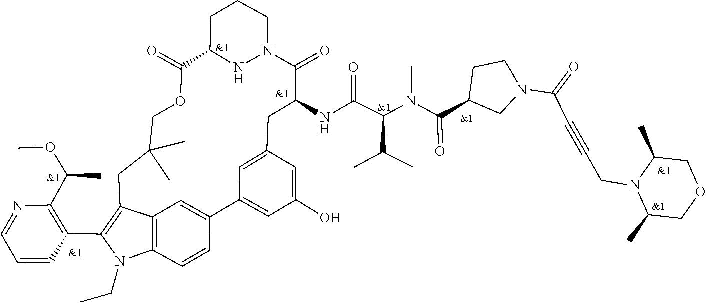 |
| BA246 | 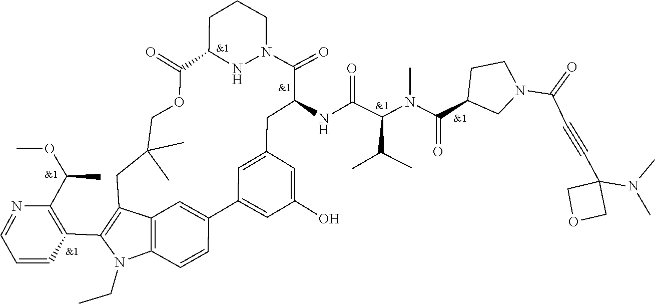 |
| BA247 | 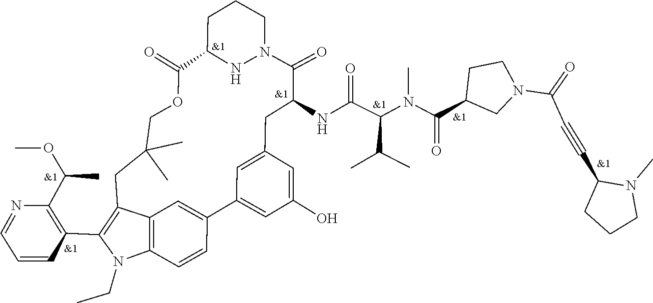 |
| BA248 | 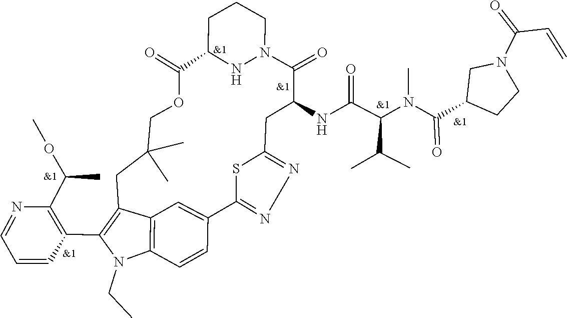 |
| BA249 | 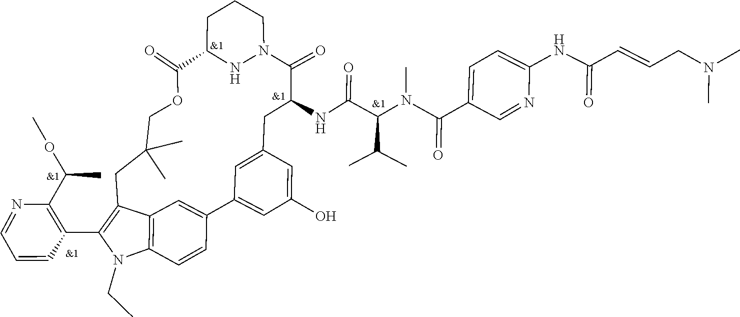 |
| BA250 | 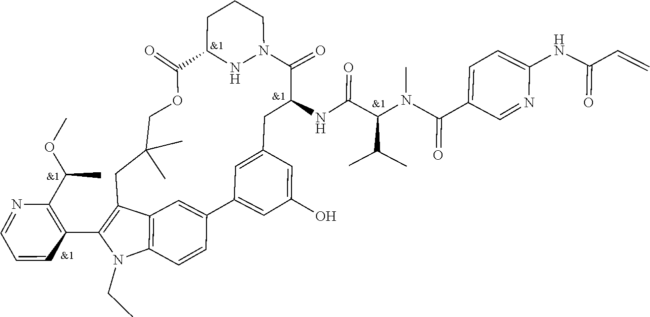 |
| BA251 | 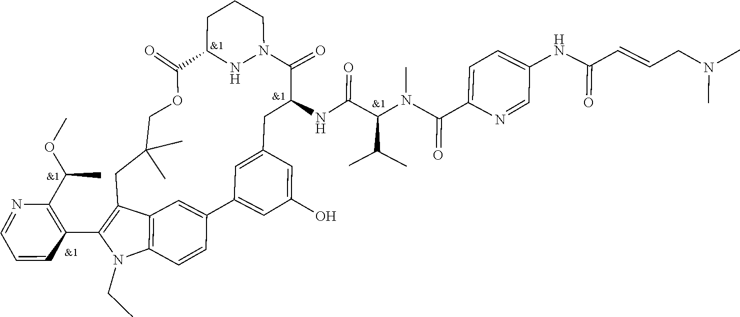 |
| BA252 | 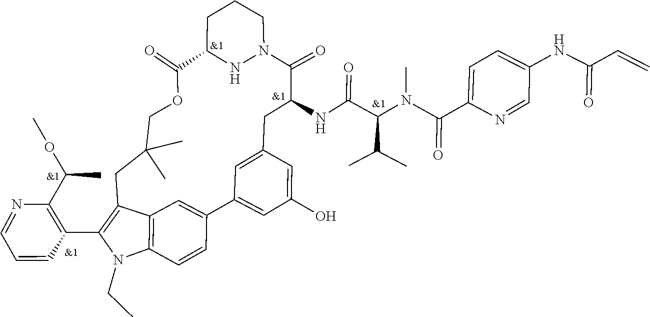 |
| BA253 | 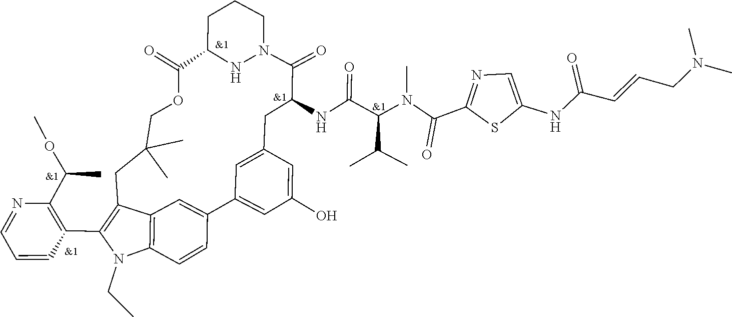 |
| BA254 | 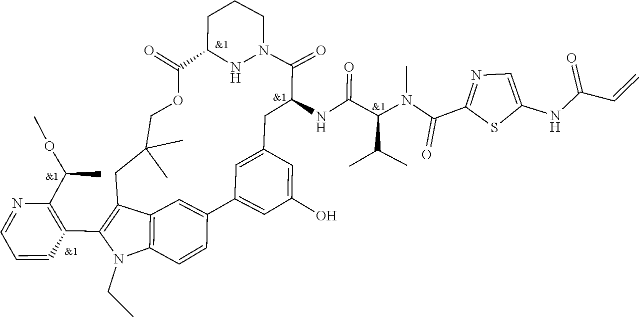 |
| BA255 | 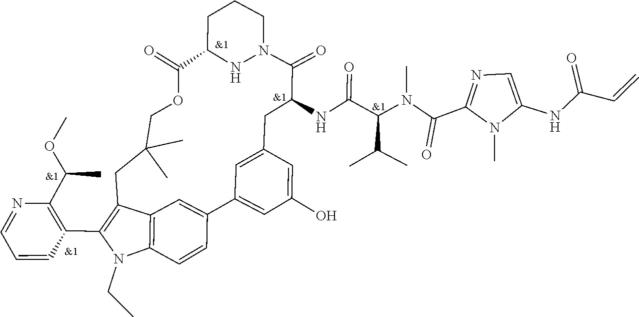 |
| BA256 | 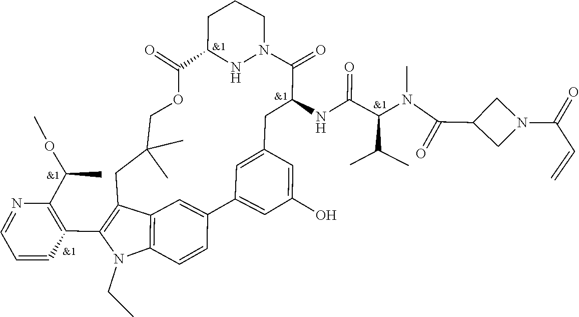 |
| BA257 | 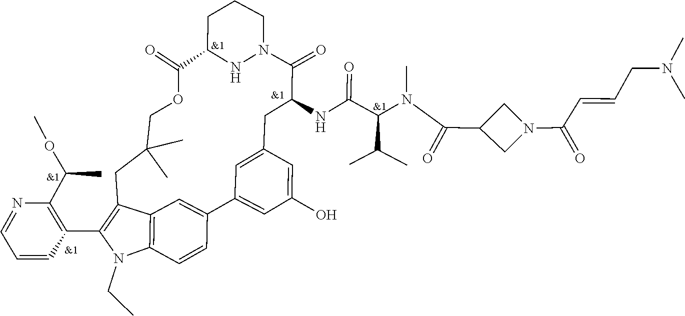 |
| BA258 | 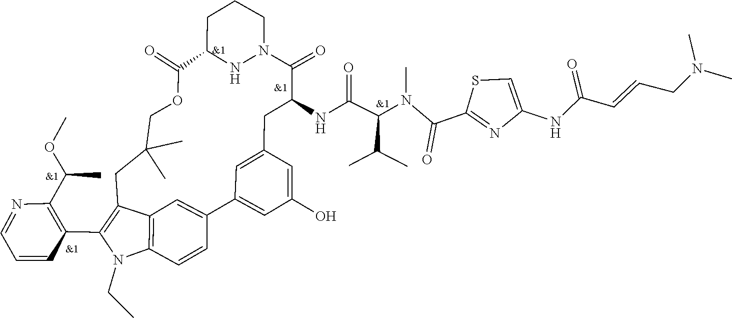 |
| BA259 | 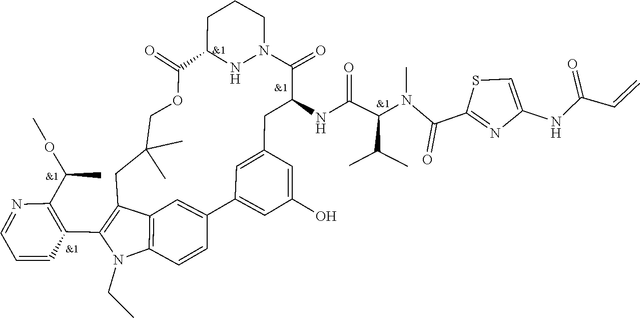 |
| BA260 | 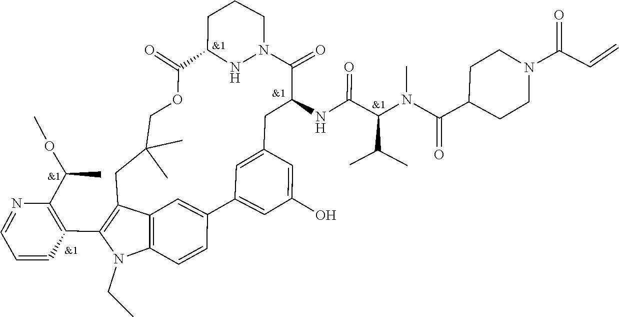 |
| BA261 | 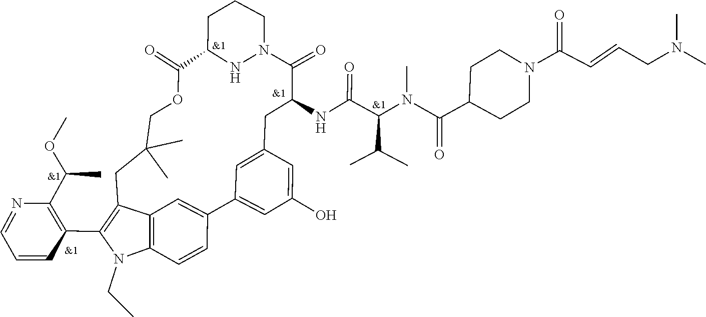 |
| BA262 | 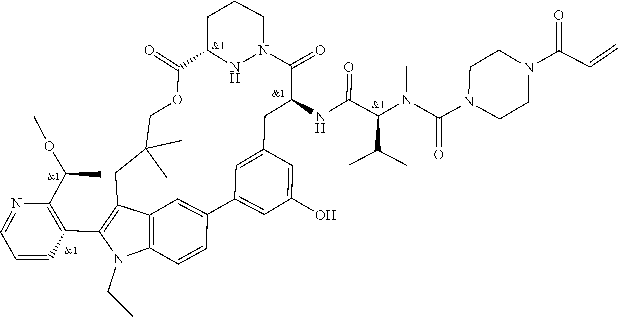 |
| BA263 | 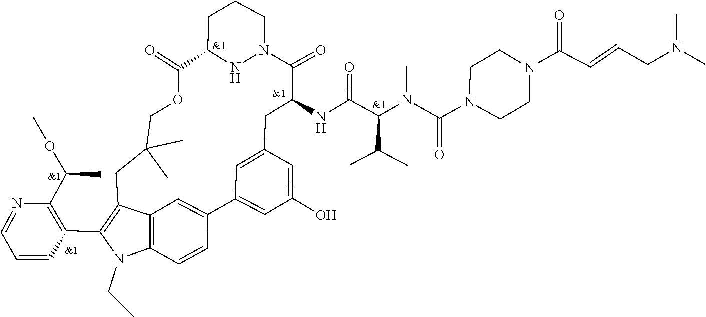 |
| BA264 | +GET,1174 |
| BA265 | 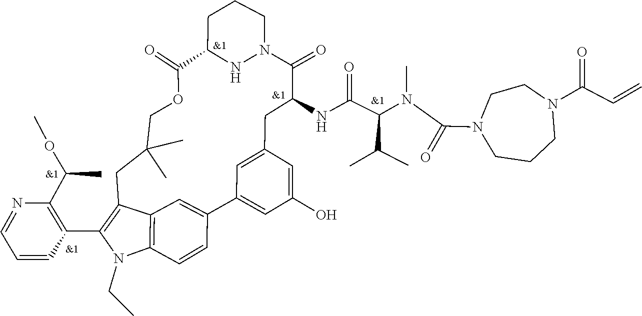 |
| BA266 | 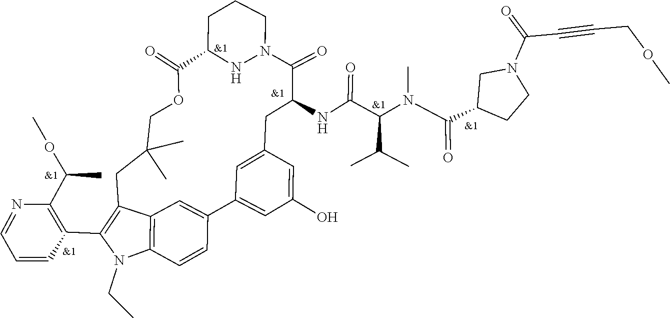 |
| BA267 | 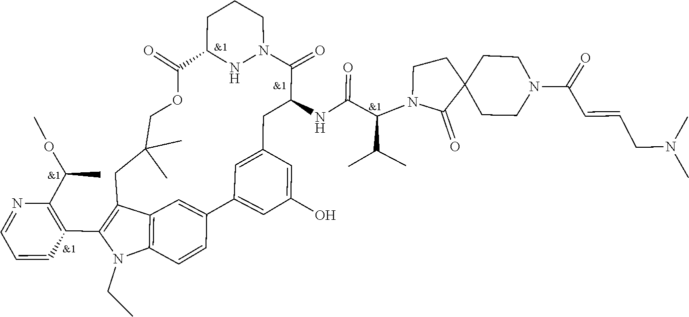 |
| BA268 | 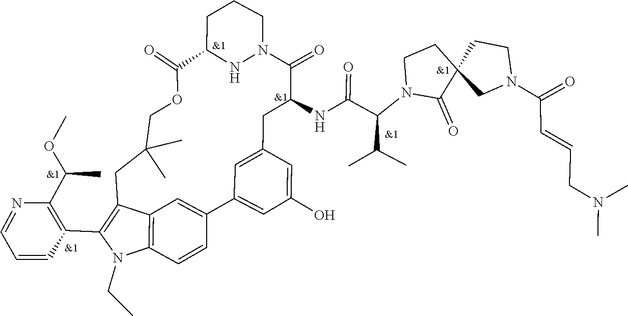 |
| BA269 | 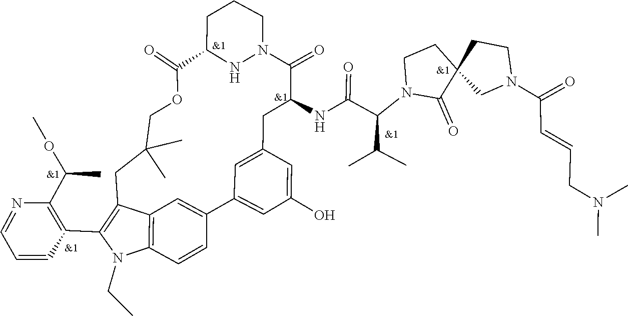 |
| BA270 | 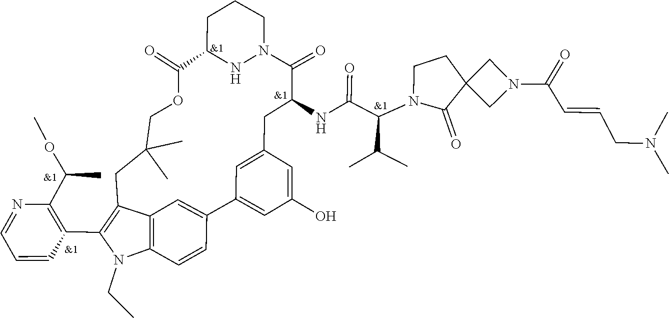 |
| BA271 | 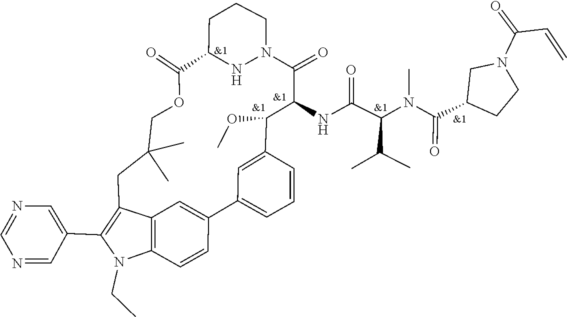 [SMILES: C=CC(=O)N1CC[C@H](C(=O)N(C)[C@H](C(=O)N[C@@H]2C(=O)N3CCC[C@H](N3)C(=O)OCC(C)(C)Cc3c(-c4cncnc4)n(CC)c4ccc(cc34)-c3cccc(c3)[C@@H]2OC)C(C)C)C1] |
| BA272 | 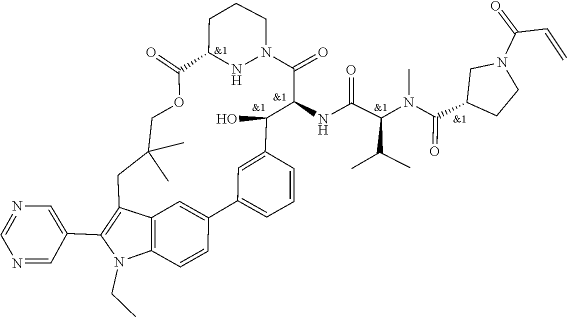 [SMILES: C=CC(=O)N1CC[C@H](C(=O)N(C)[C@H](C(=O)N[C@@H]2C(=O)N3CCC[C@H](N3)C(=O)OCC(C)(C)Cc3c(-c4cncnc4)n(CC)c4ccc(cc34)-c3cccc(c3)[C@H]2O)C(C)C)C1] |
| BA273 | 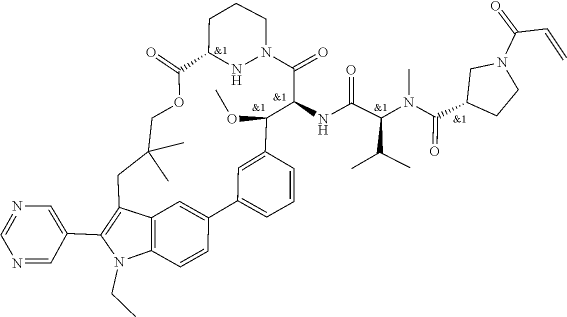 [SMILES: C=CC(=O)N1CC[C@H](C(=O)N(C)[C@H](C(=O)N[C@@H]2C(=O)N3CCC[C@H](N3)C(=O)OCC(C)(C)Cc3c(-c4cncnc4)n(CC)c4ccc(cc34)-c3cccc(c3)[C@H]2OC)C(C)C)C1] |
| BA274 | 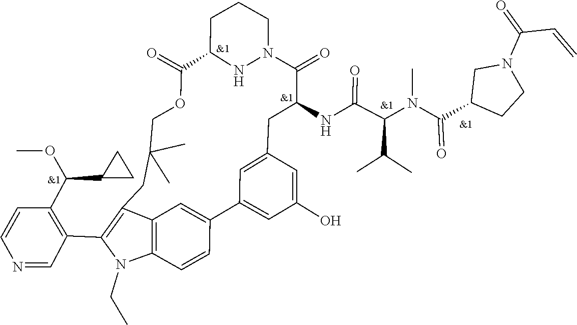 [SMILES: C=CC(=O)N1CC[C@H](C(=O)N(C)[C@H](C(=O)N[C@H]2Cc3cc(O)cc(c3)-c3ccc4c(c3)c(c(-c3cnccc3[C@@H](OC)C3CC3)n4CC)CC(C)(C)COC(=O)[C@@H]3CCCN(N3)C2=O)C(C)C)C1] |
| BA275 | 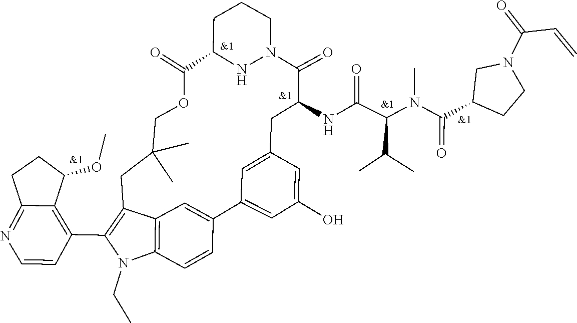 [SMILES: C=CC(=O)N1CC[C@H](C(=O)N(C)[C@H](C(=O)N[C@H]2Cc3cc(O)cc(c3)-c3ccc4c(c3)c(c(-c3ccnc5c3[C@@H](OC)CC5)n4CC)CC(C)(C)COC(=O)[C@@H]3CCCN(N3)C2=O)C(C)C)C1] |
| BA276 | 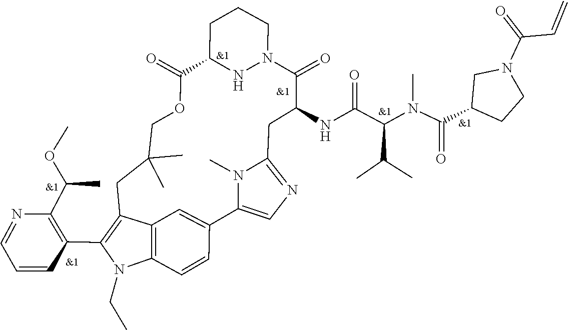 |
| BA277 | 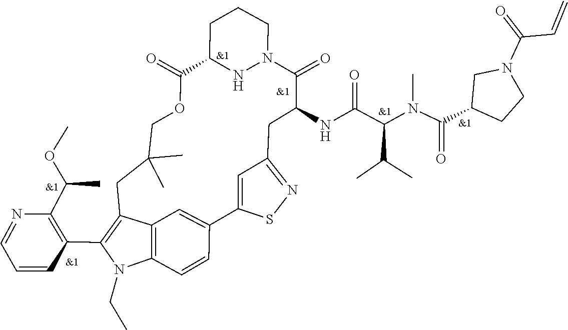 |
| BA278 | 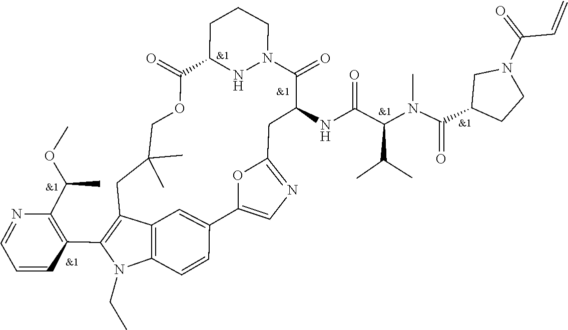 |
| BA279 | 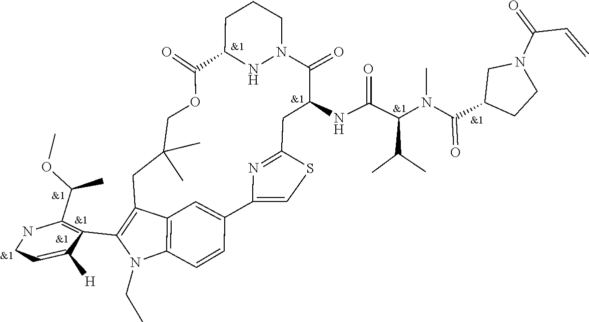 |
| BA280 | 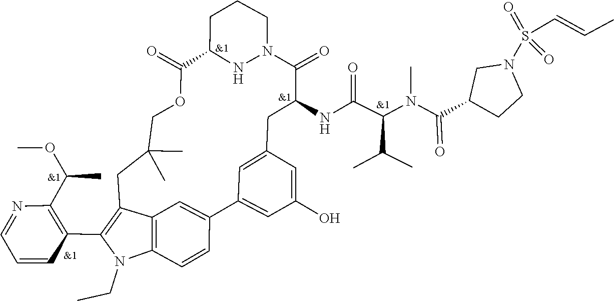 |
| BA281 | 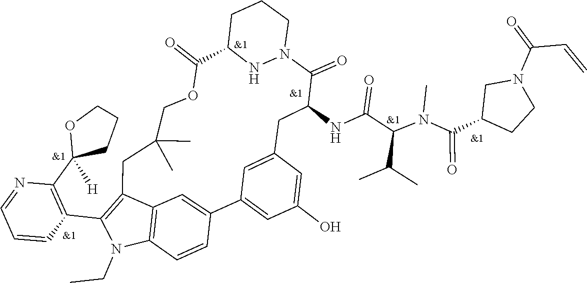 |
| BA282 | 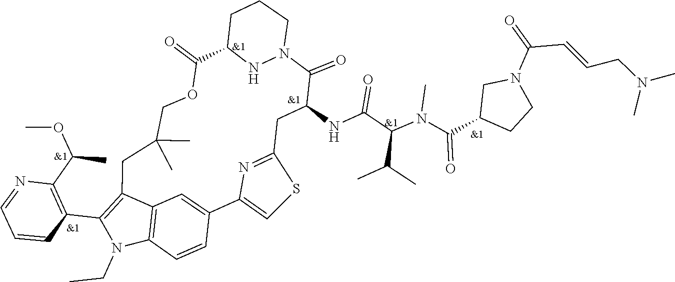 |
| BA283 | 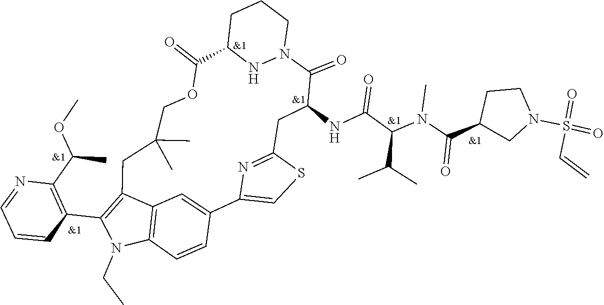 |
| BA284 | 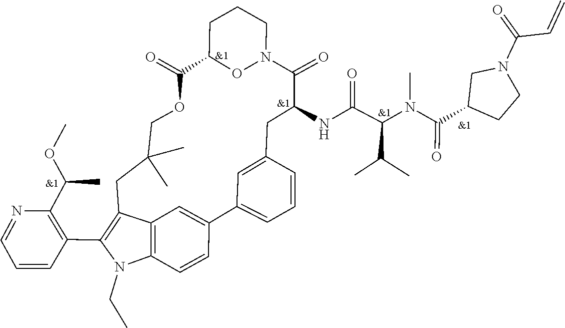 |
| BA285 | 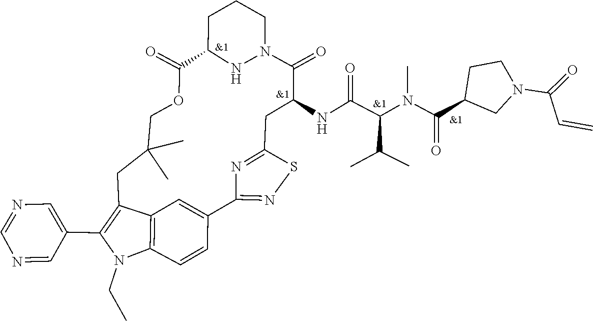 [SMILES: C=CC(=O)N1CC[C@H](C(=O)N(C)[C@H](C(=O)N[C@H]2Cc3nc(ns3)-c3ccc4c(c3)c(c(-c3cncnc3)n4CC)CC(C)(C)COC(=O)[C@@H]3CCCN(N3)C2=O)C(C)C)C1] |
| BA286 | 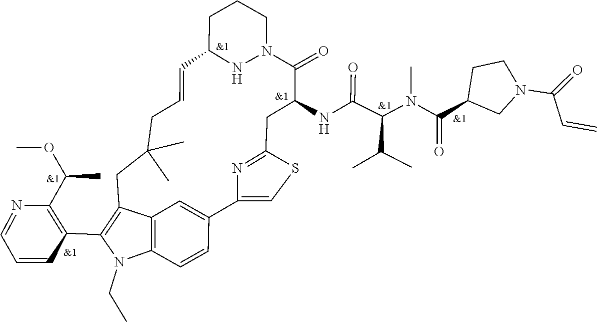 |
| BA287 | 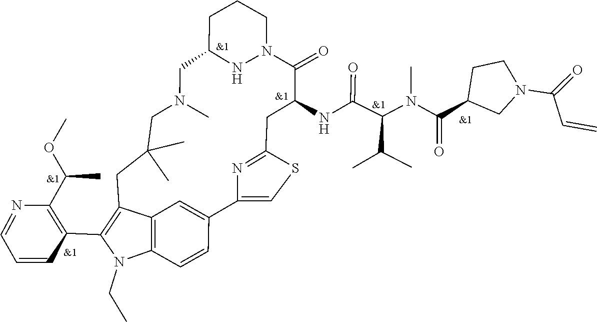 |
| BA288 | 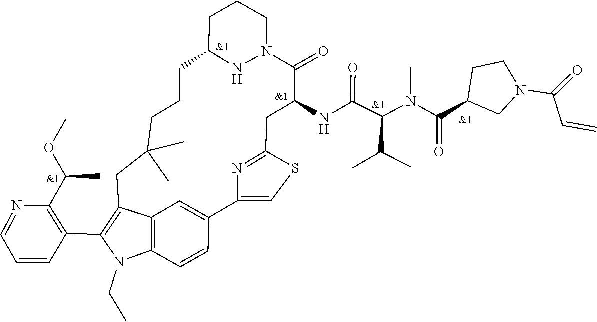 [SMILES: C=CC(=O)N1CC[C@H](C(=O)N(C)[C@H](C(=O)N[C@H]2Cc3nc(cs3)-c3ccc4c(c3)c(c(-c3cccnc3[C@H](C)OC)n4CC)CC(C)(C)CCC[C@@H]3CCCN(N3)C2=O)C(C)C)C1] |
| BA289 | 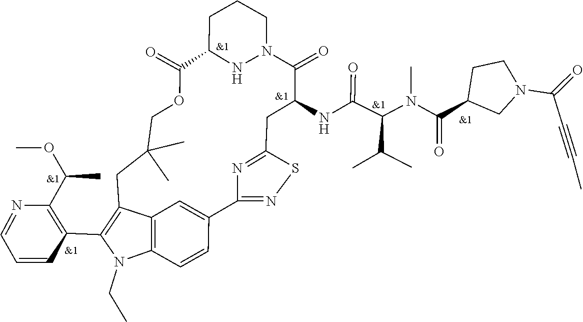 |
| BA290 | 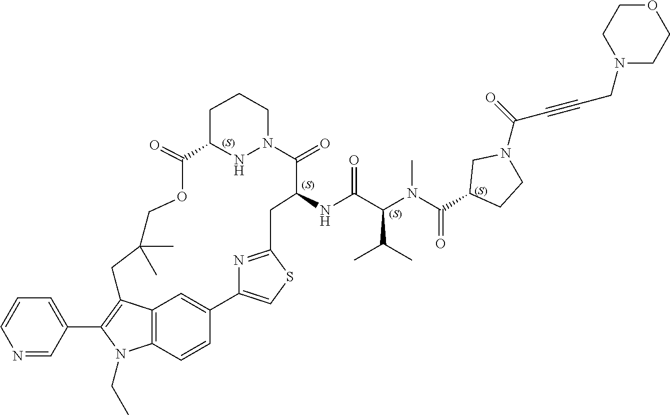 |
| BA291 | 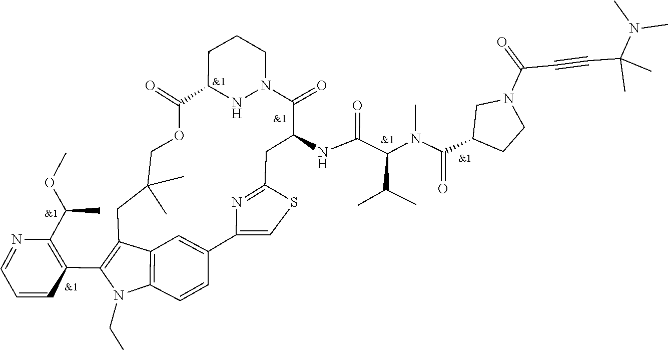 |
| BA292 | 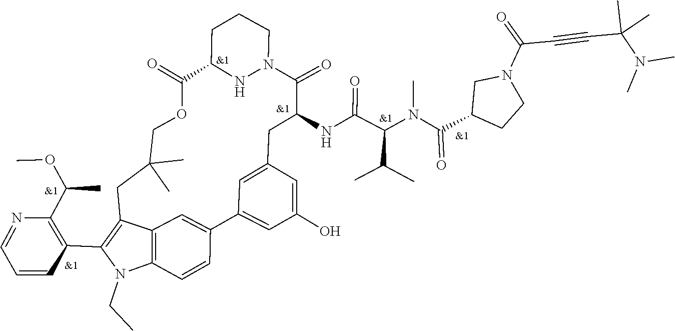 |
| BA293 | 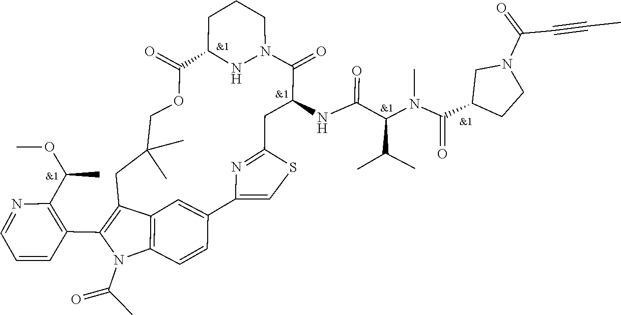 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(C)=O)CC(C)(C)COC(=O)[C@@H]3CCCN(N3)C2=O)C(C)C)C1] |
| BA294 | 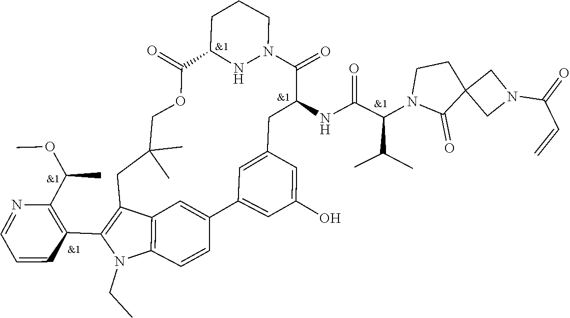 |
| BA295 | 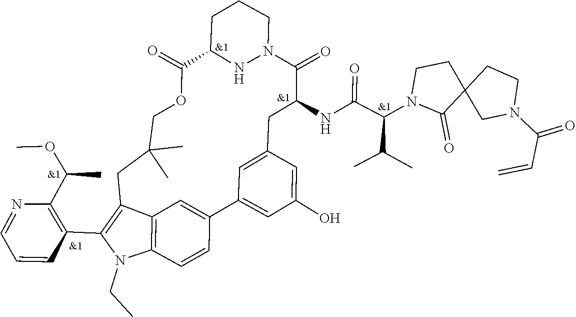 |
| BA296 | 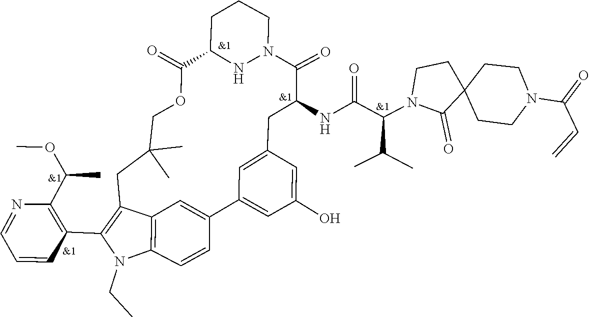 |
| BA297 | 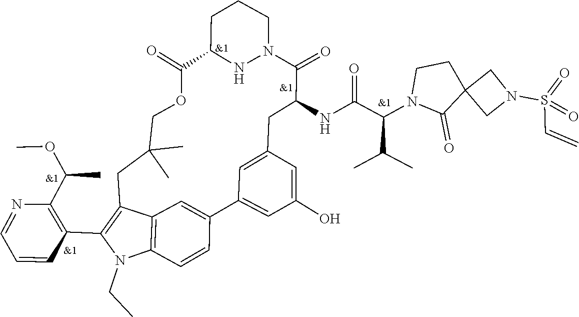 |
| BA298 | 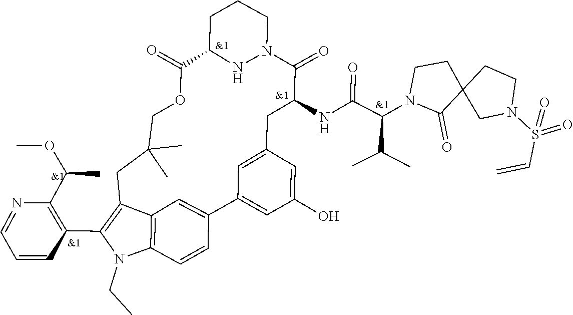 |
| BA299 | 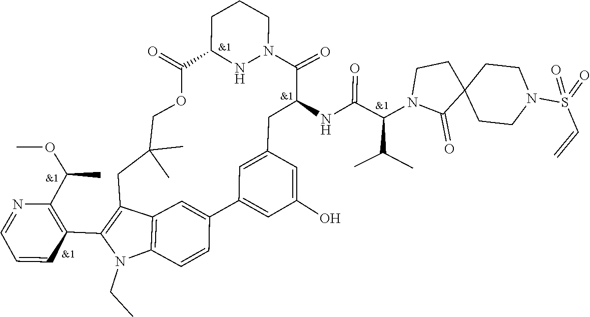 |
| BA300 | 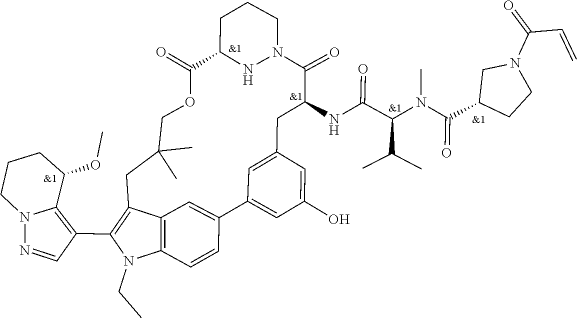 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CCC5)n4CC)CC(C)(C)COC(=O)[C@@H]3CCCN(N3)C2=O)C(C)C)C1] |
| BA301 | 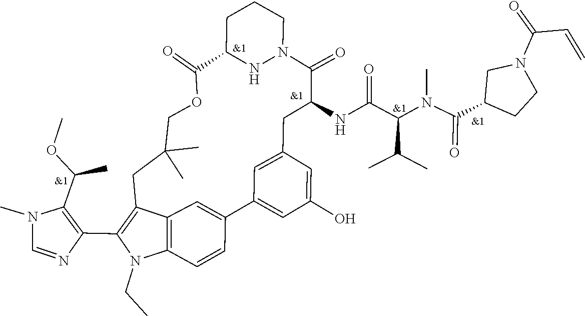 [SMILES: C=CC(=O)N1CC[C@H](C(=O)N(C)[C@H](C(=O)N[C@H]2Cc3cc(O)cc(c3)-c3ccc4c(c3)c(c(-c3ncn(C)c3[C@H](C)OC)n4CC)CC(C)(C)COC(=O)[C@@H]3CCCN(N3)C2=O)C(C)C)C1] |
| BA302 | 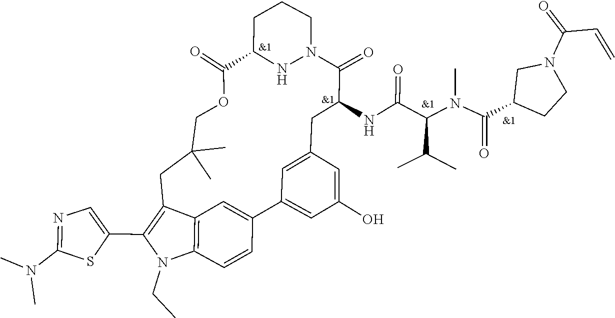 [SMILES: C=CC(=O)N1CC[C@H](C(=O)N(C)[C@H](C(=O)N[C@H]2Cc3cc(O)cc(c3)-c3ccc4c(c3)c(c(-c3cnc(N(C)C)s3)n4CC)CC(C)(C)COC(=O)[C@@H]3CCCN(N3)C2=O)C(C)C)C1] |
| BA303 | 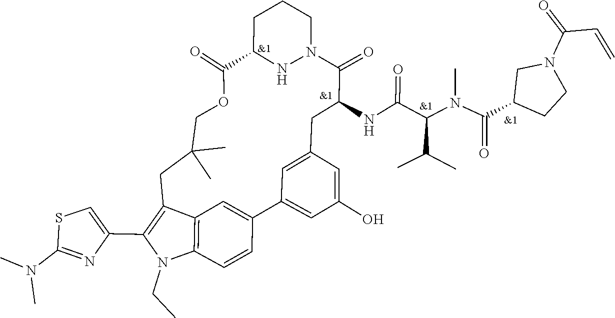 [SMILES: C=CC(=O)N1CC[C@H](C(=O)N(C)[C@H](C(=O)N[C@H]2Cc3cc(O)cc(c3)-c3ccc4c(c3)c(c(-c3csc(N(C)C)n3)n4CC)CC(C)(C)COC(=O)[C@@H]3CCCN(N3)C2=O)C(C)C)C1] |
| BA304 | 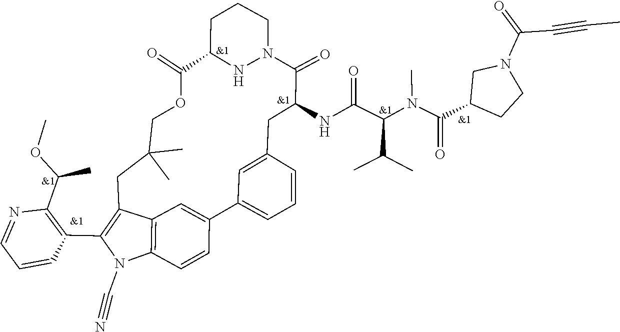 |
| BA305 | 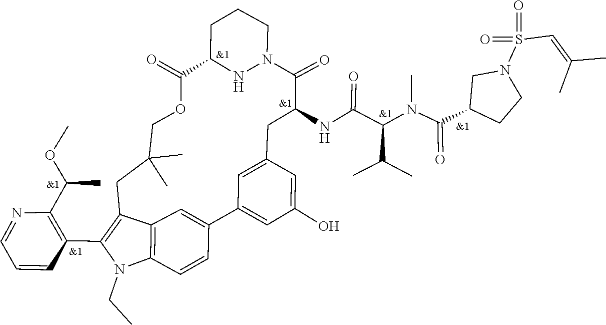 |
| BA306 | 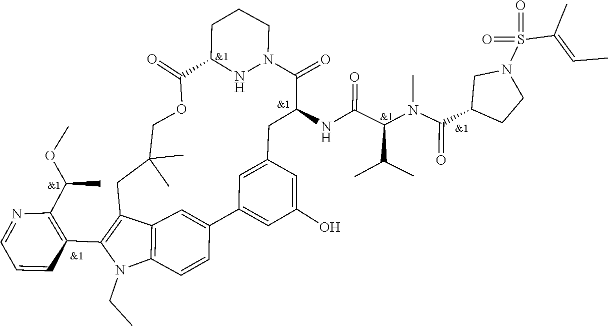 |
| BA307 | 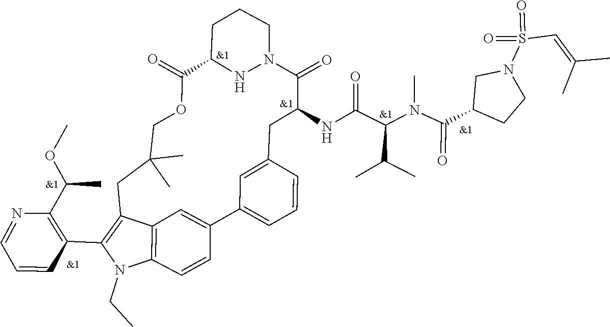 |
| BA308 | 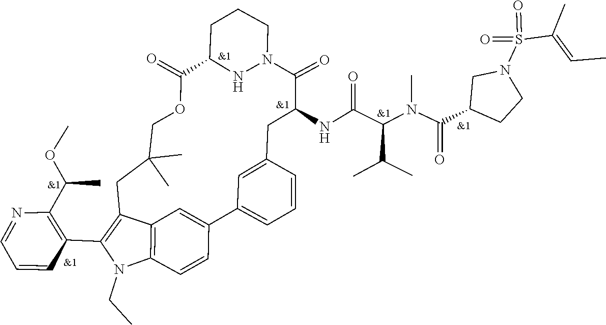 |
| BA309 | 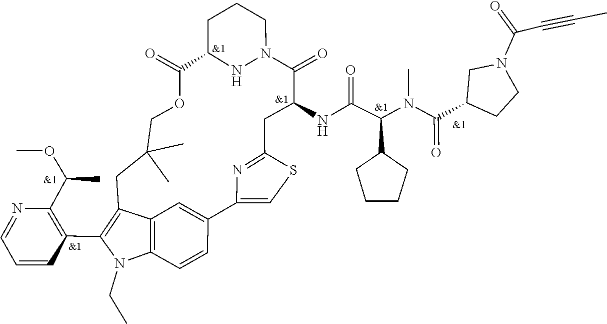 |
| BA310 | 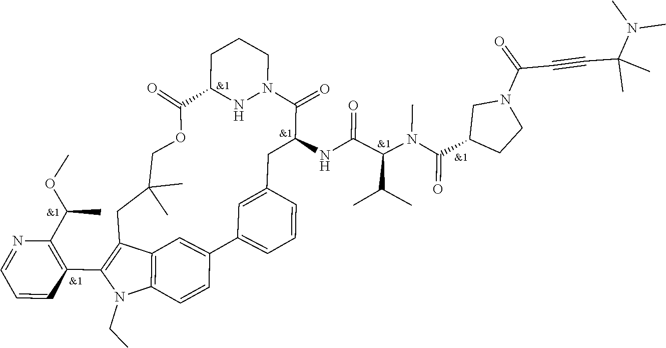 |
| BA311 | 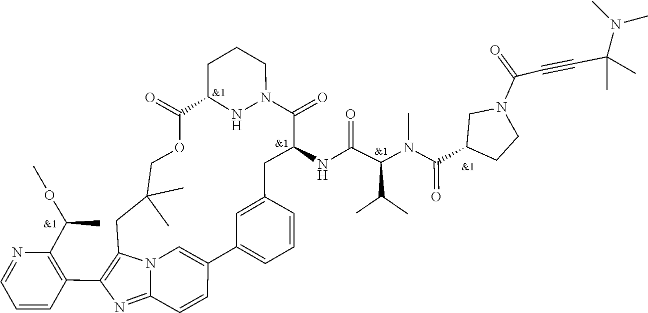 [SMILES: CO[C@@H](C)c1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CC(C)(C)N(C)C)C1)Cc1cccc-3c1] |
| BA312 | 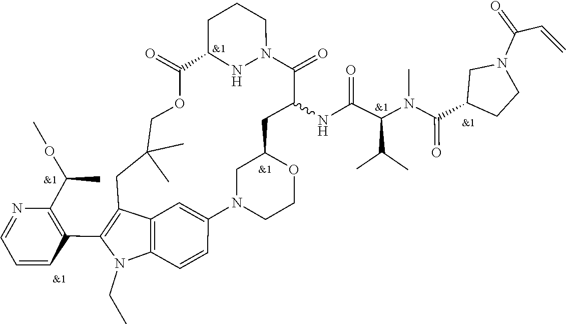 |
| BA313 | 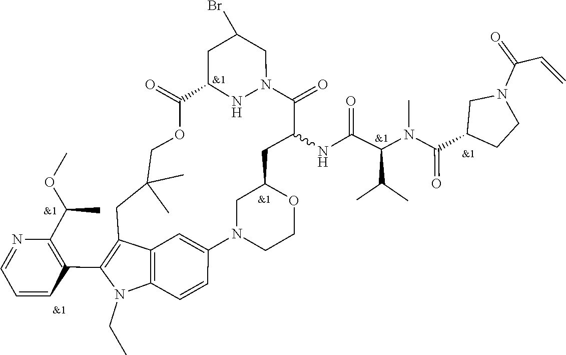 |
| BA314 | 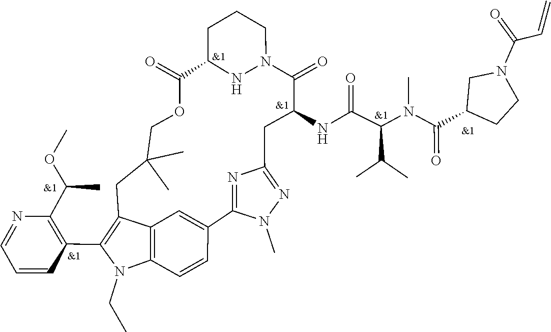 |
| BA316 | 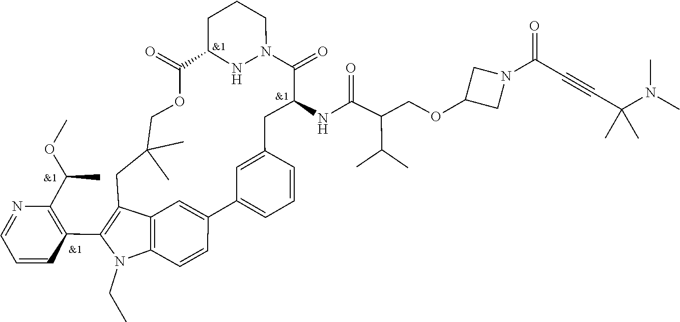 |
| BA317 | 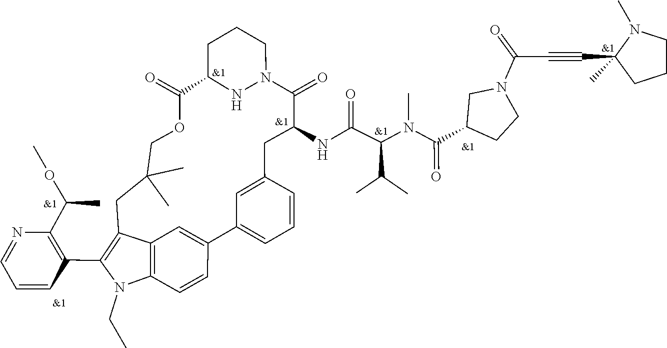 |
| BA318 | 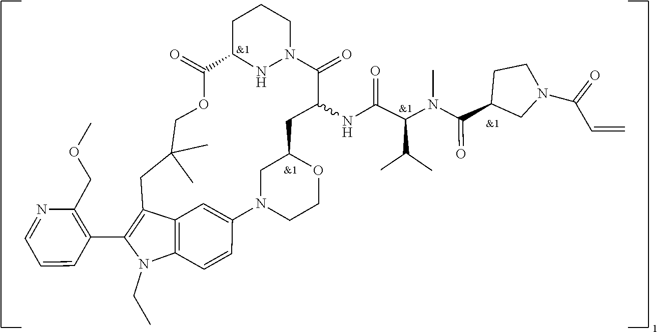 |
| BA319 | 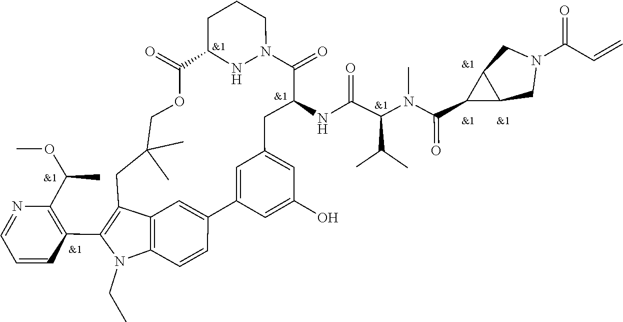 |
| BA320 | 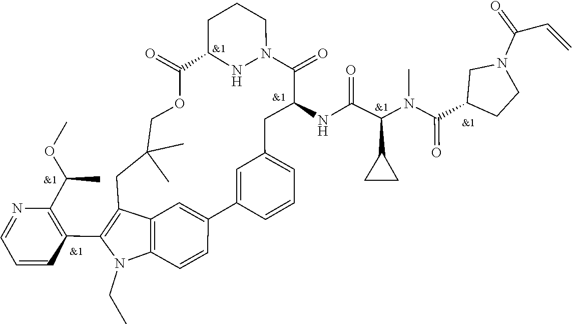 |
| BA321 | 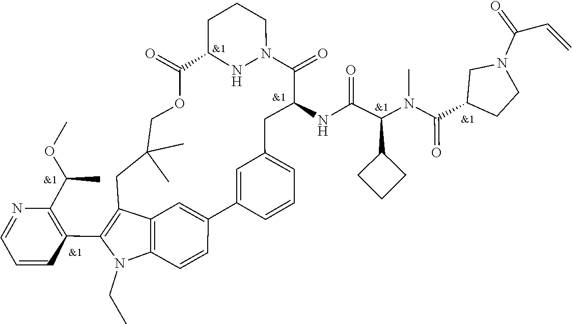 |
| BA322 | 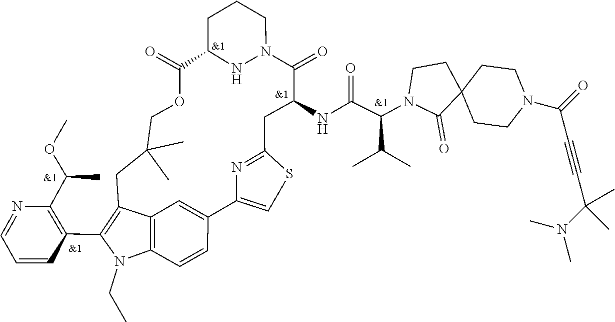 |
| BA323 | 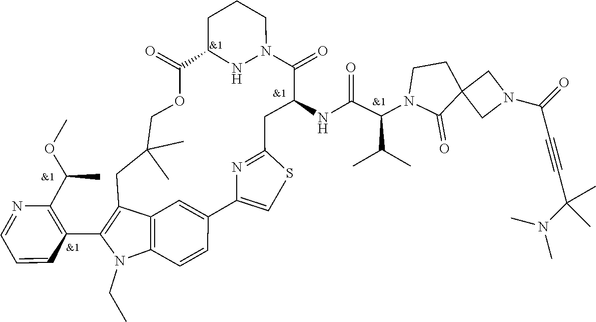 |
| BA324 | 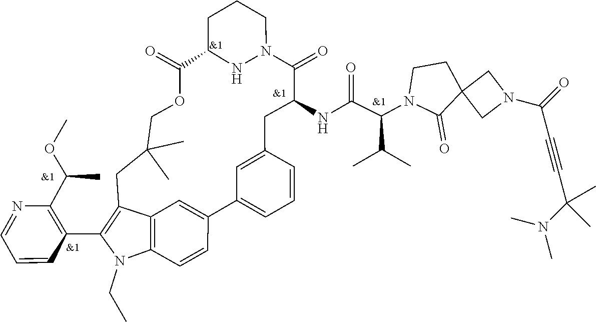 |
| BA325 | 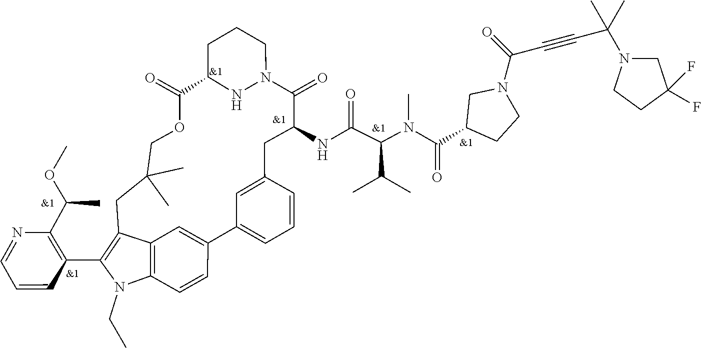 |
| BA326 | 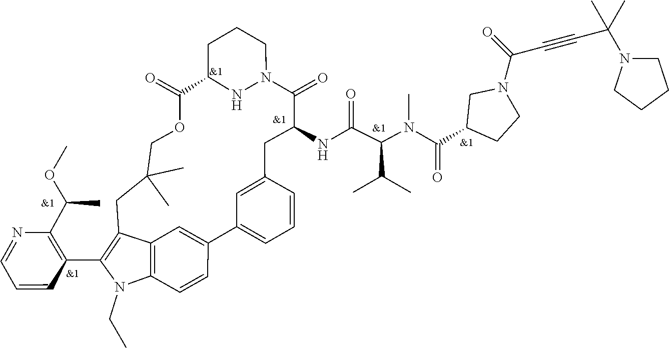 |
| BA327 | 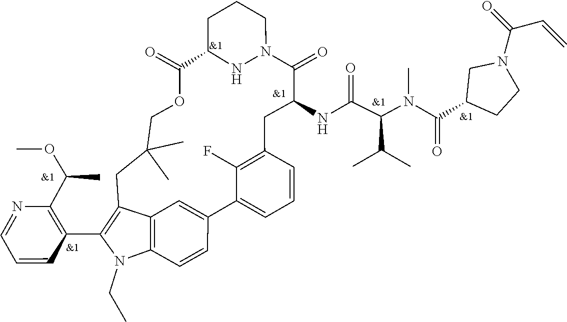 |
| BA328 | 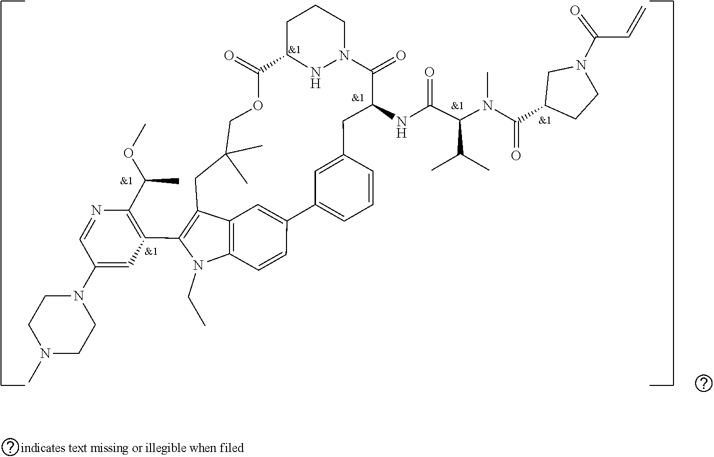 |
| BA329 | 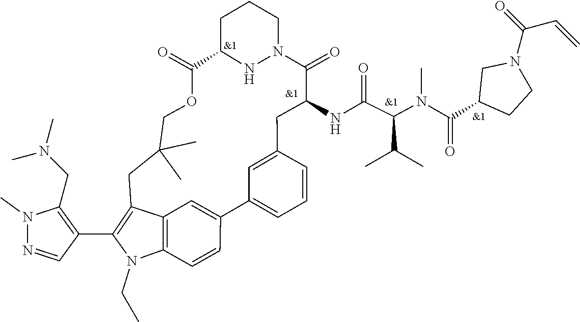 [SMILES: C=CC(=O)N1CC[C@H](C(=O)N(C)[C@H](C(=O)N[C@H]2Cc3cccc(c3)-c3ccc4c(c3)c(c(-c3cnn(C)c3CN(C)C)n4CC)CC(C)(C)COC(=O)[C@@H]3CCCN(N3)C2=O)C(C)C)C1] |
| BA330 | 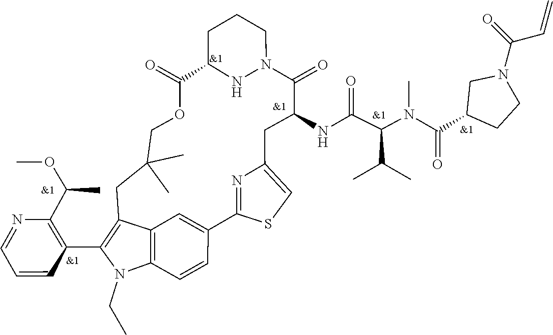 |
| BA331 | 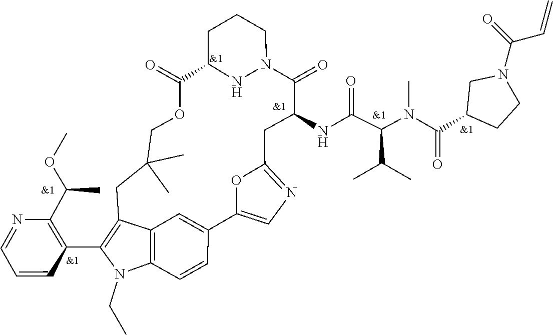 |
| BA332 | 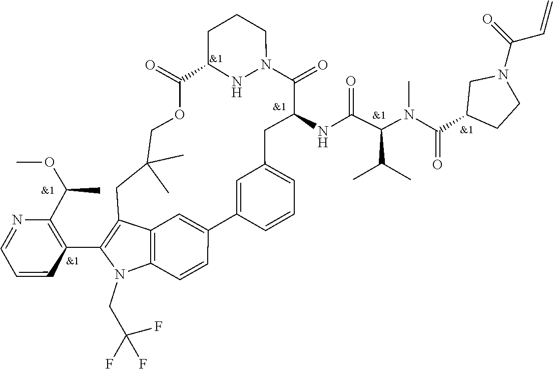 |
| BA333 | 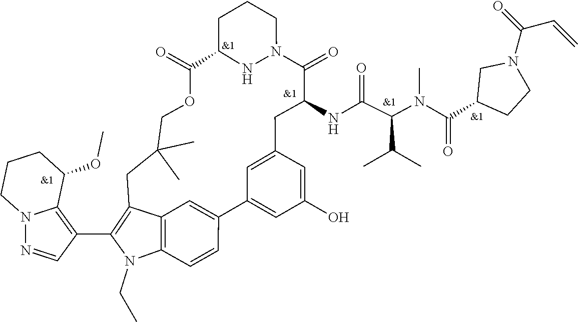 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CCC5)n4CC)CC(C)(C)COC(=O)[C@@H]3CCCN(N3)C2=O)C(C)C)C1] |
| BA334 | 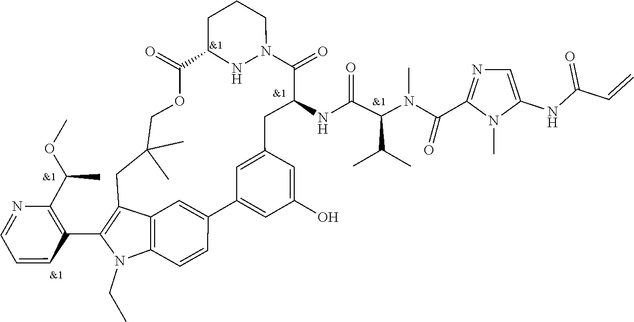 |
| BA335 | 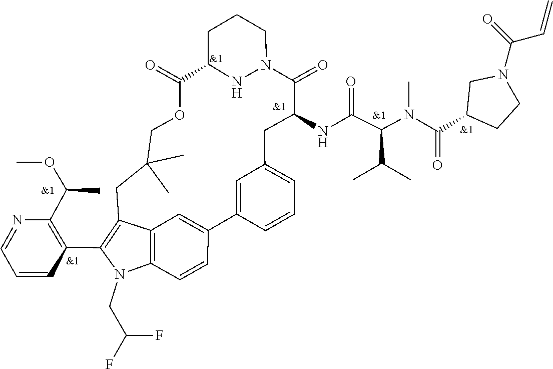 |
| BA336 | 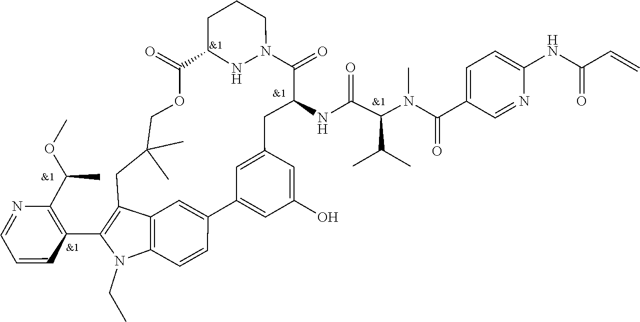 |
| BA337 | 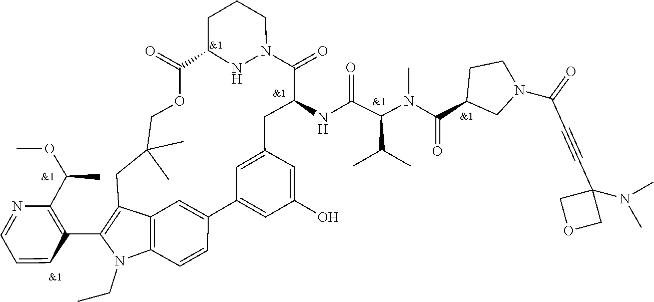 |
| BA338 | 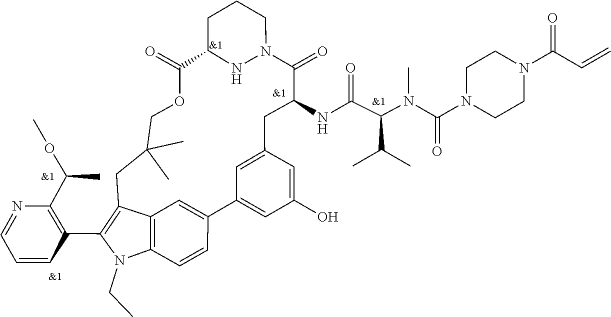 |
| BA339 | 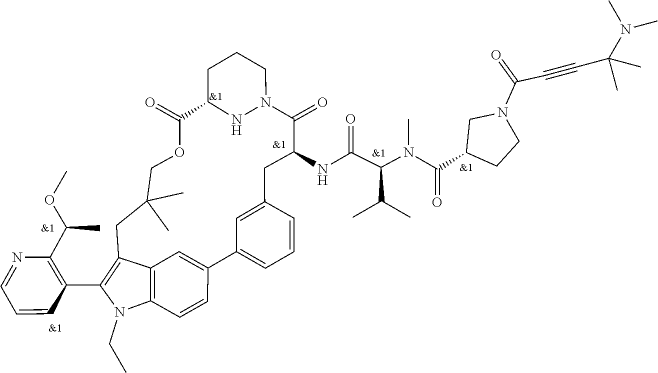 |
| BA340 | 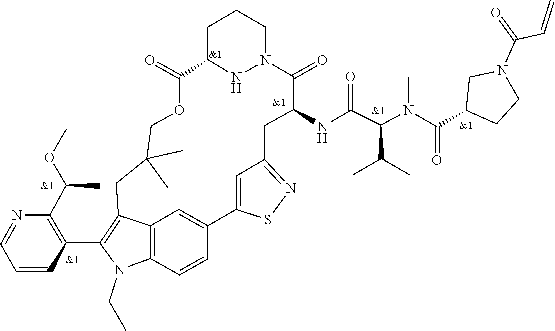 |
| BA341 | 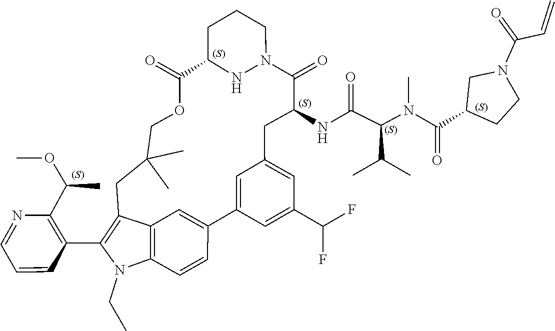 |
| BA342 | 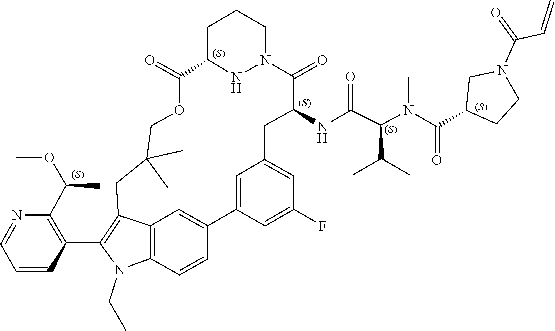 |
| BA343 | 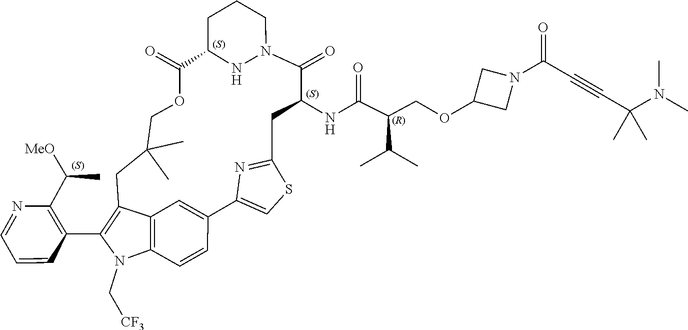 |
| BA344 | 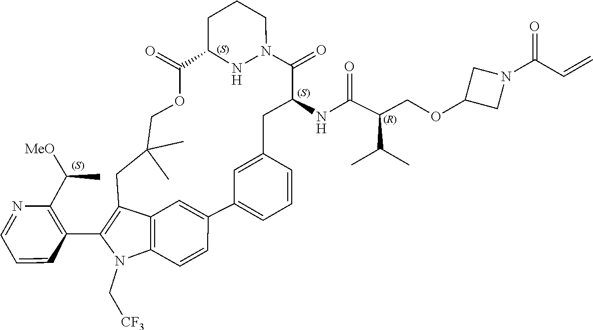 |
| BA345 | 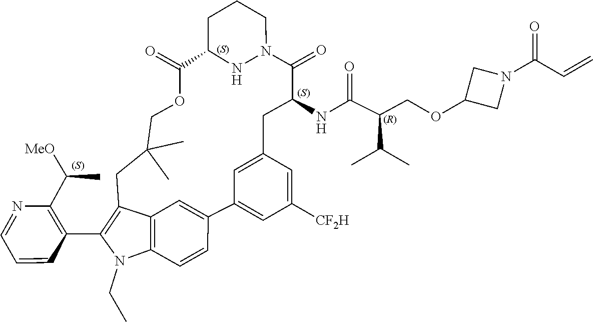 |
| BA346 | |
| BA347 | 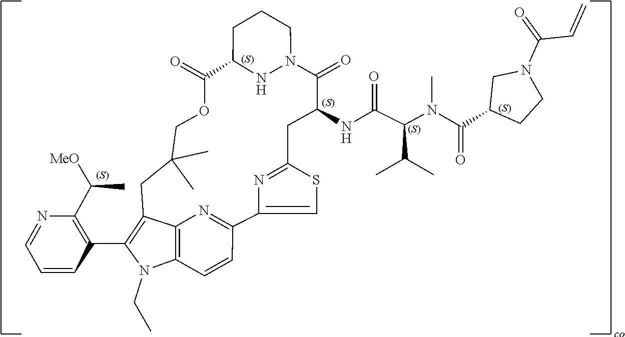 |
| BA348 | 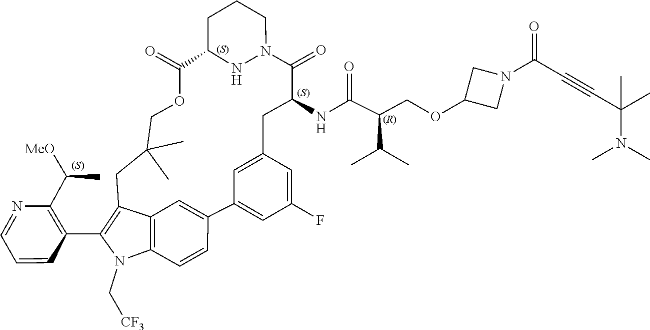 |
| BA349 | 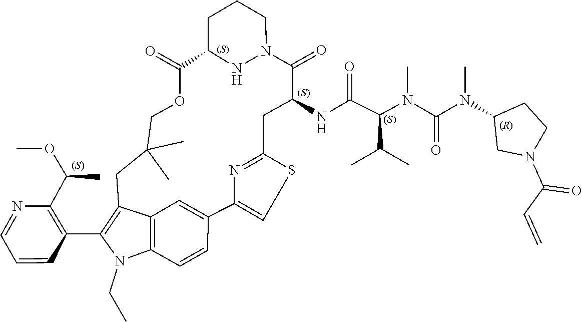 |
| BA350 | 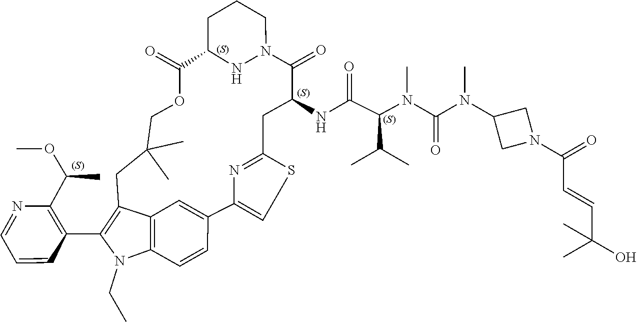 |
| BA351 | 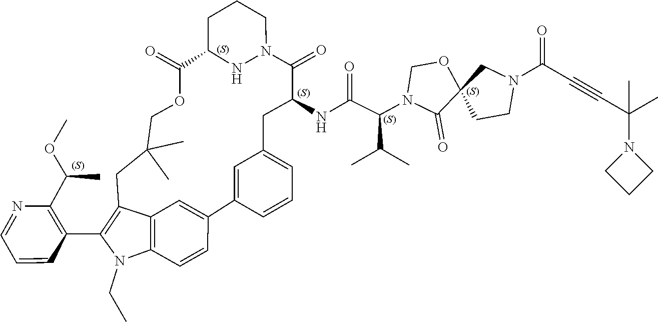 |
| BA352 | 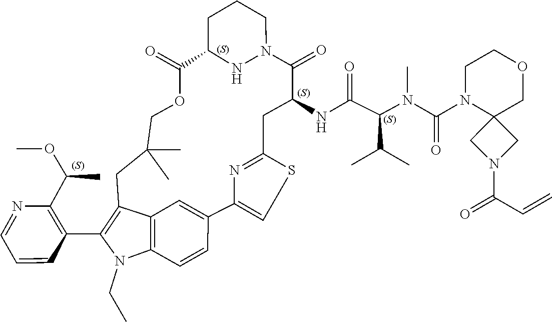 |
| BA353 | 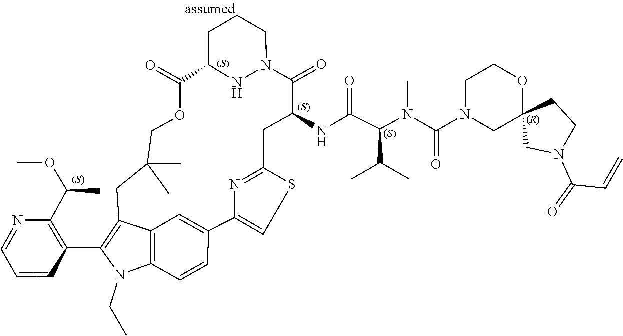 |
| BA354 | 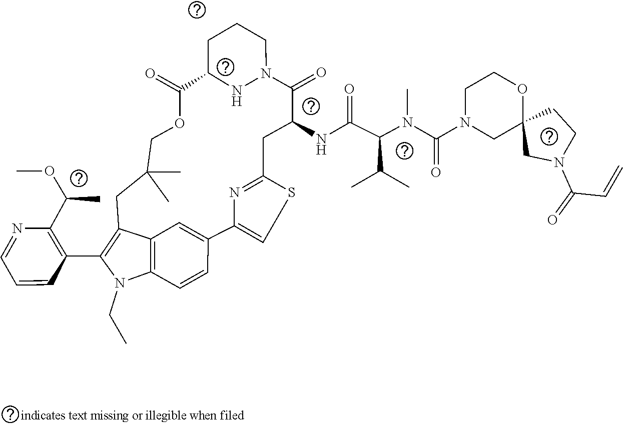 |
| BA355 | 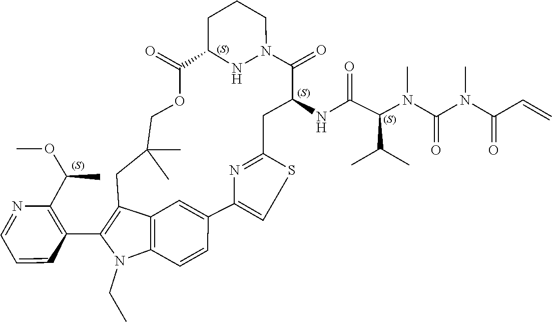 |
| BA356 | 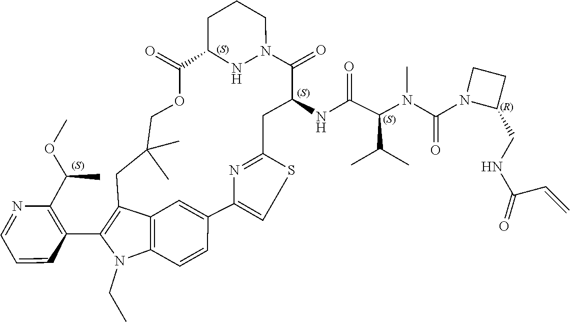 |
| BA357 | 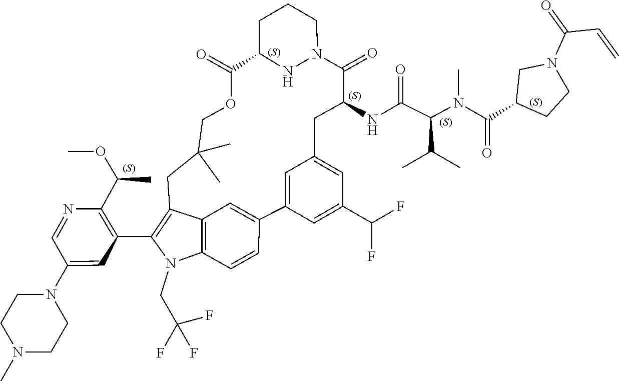 |
| BA358 | 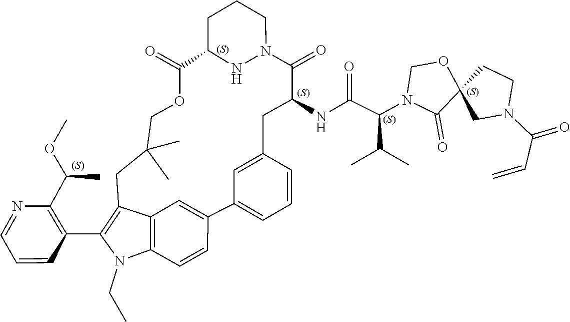 |
| BA359 | |
| BA360 | 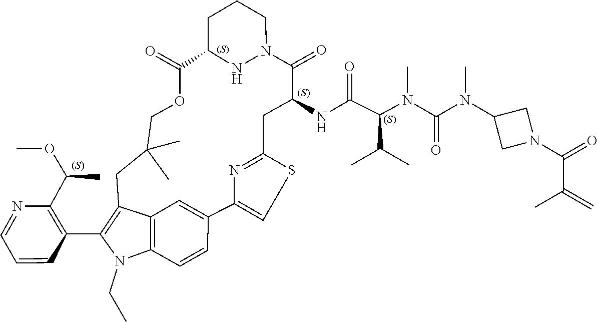 |
| BA361 | 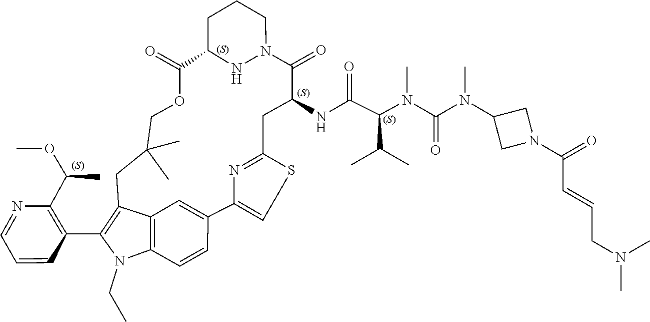 |
| BA362 | 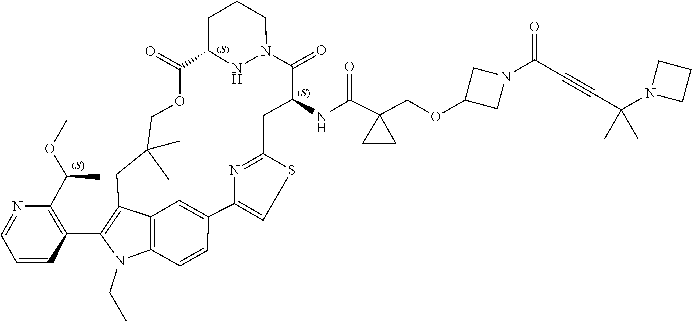 |
| BA363 | 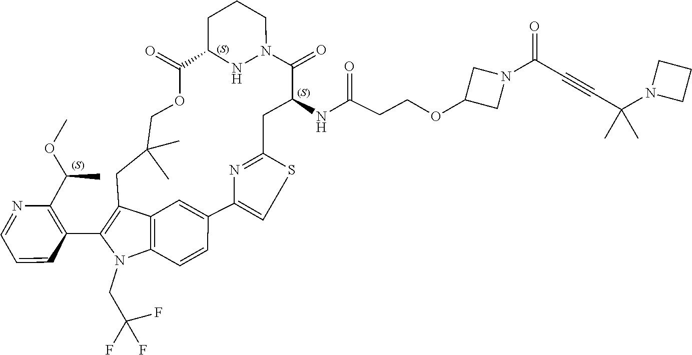 |
| BA364 | 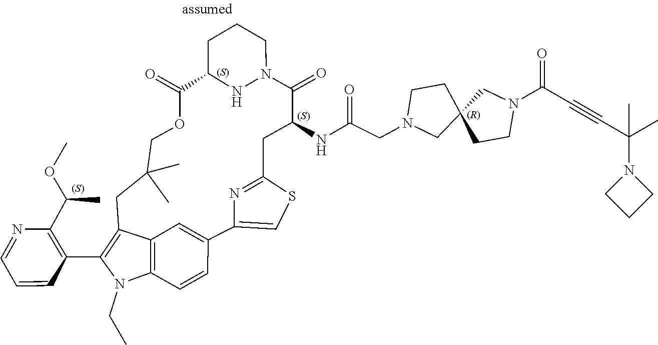 |
| BA365 | 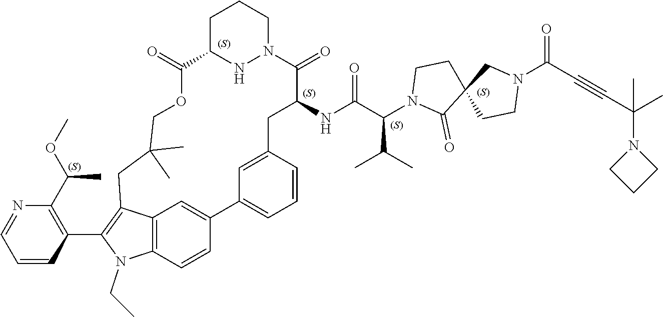 |
| BA366 | 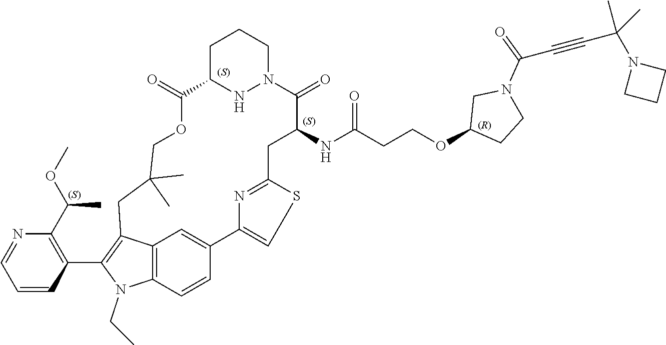 |
| BA367 | 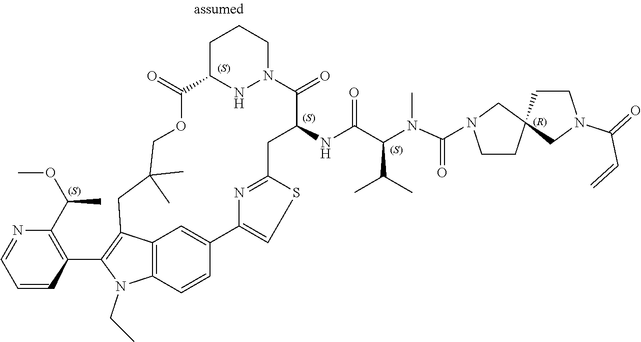 |
| BA368 | 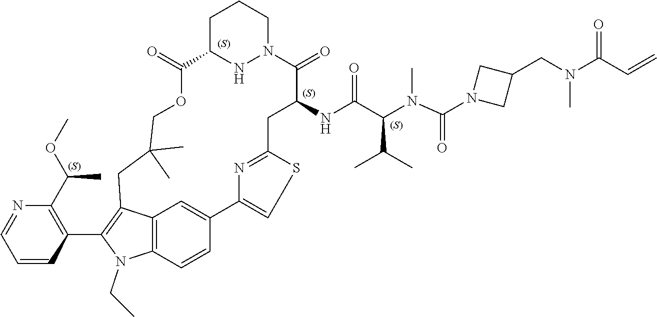 |
| BA369 | 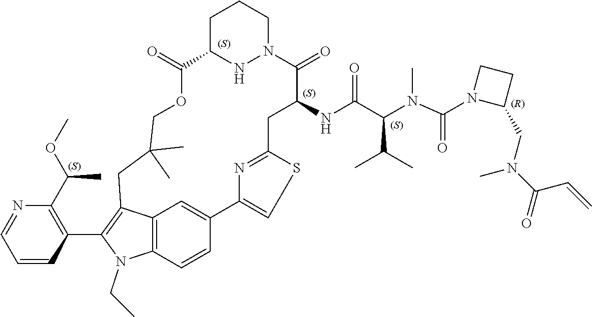 |
| BA370 | 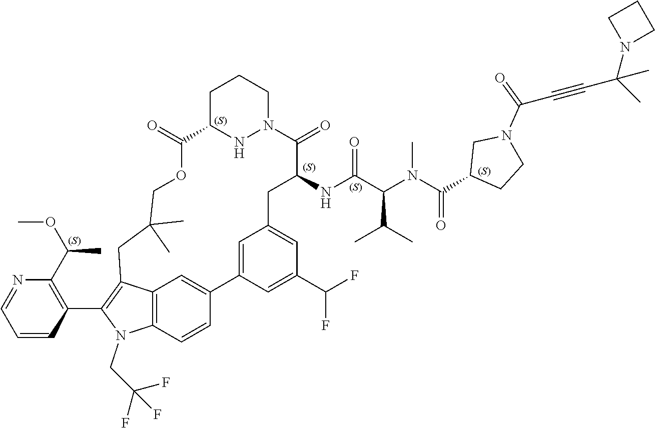 |
| BA371 | 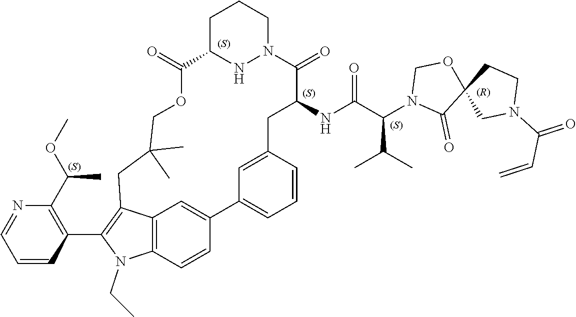 |
| BA372 | 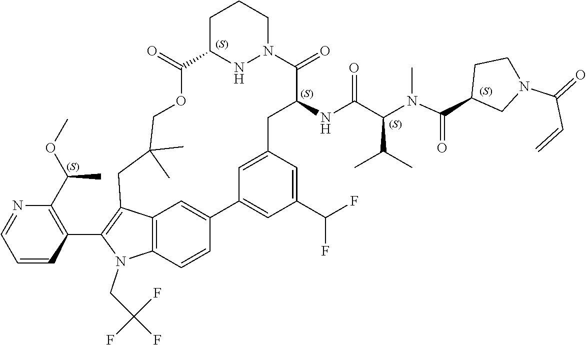 |
| BA373 | 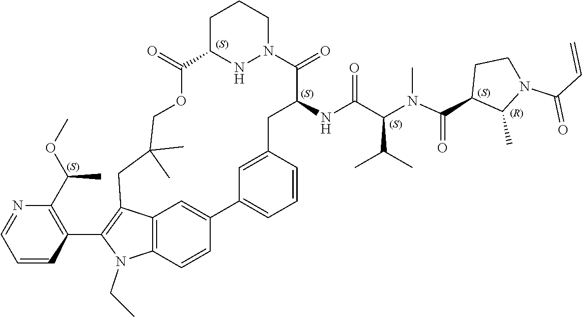 |
| BA374 | |
| BA375 | 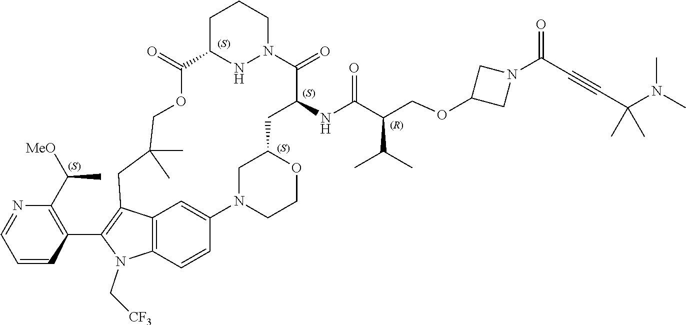 |
| BA376 | 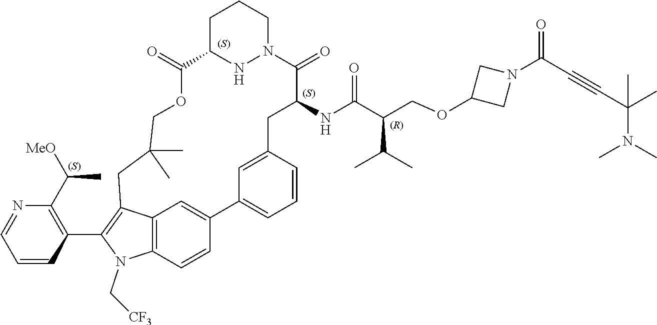 |
| BA377 | 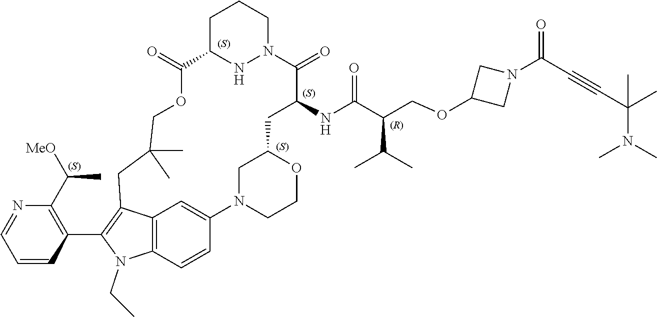 |
| BA378 | |
| BA379 | 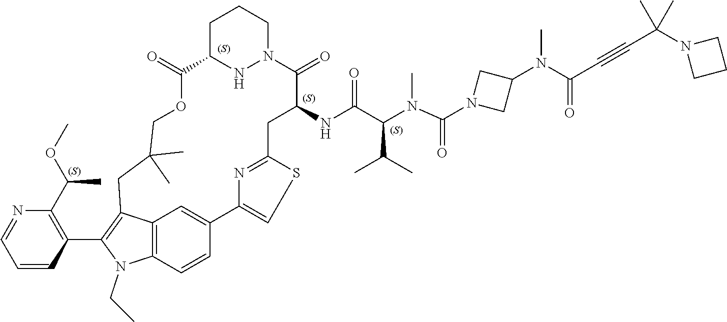 |
| BA380 | 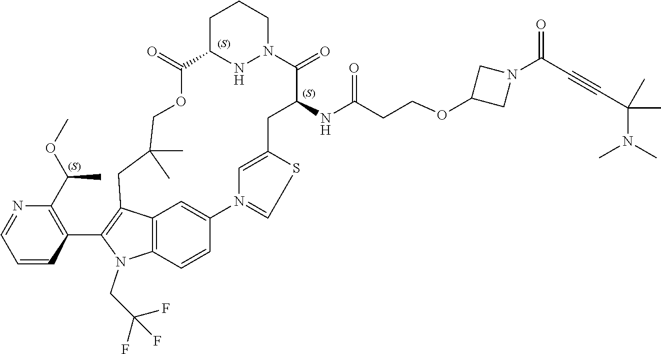 |
| BA381 | 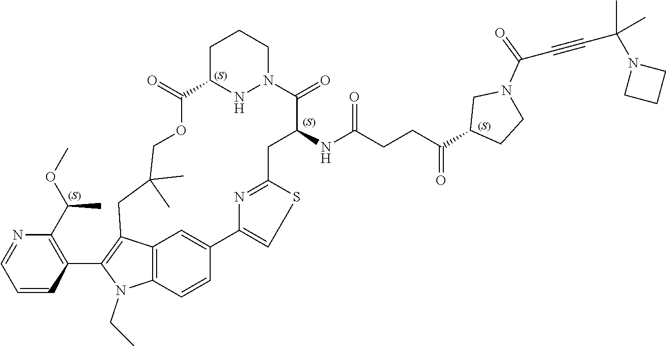 |
| BA382 | 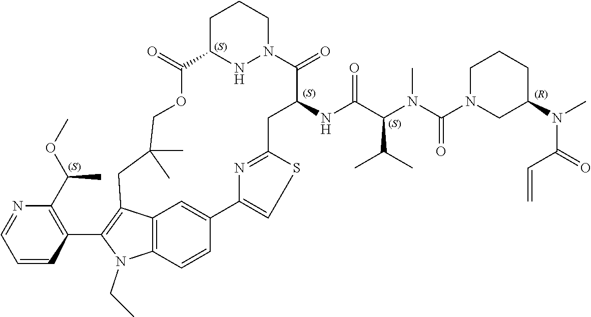 |
| BA383 | 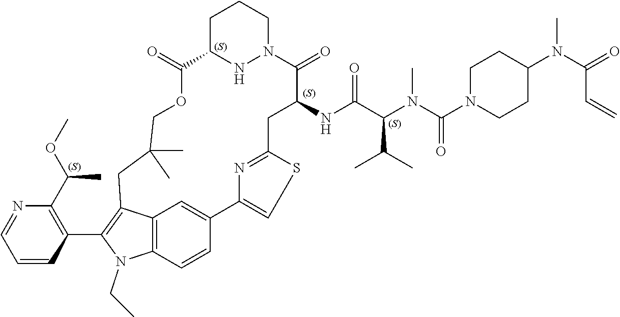 |
| BA384 | 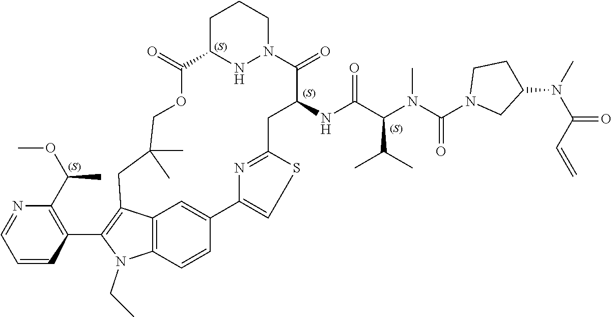 |
| BA385 | 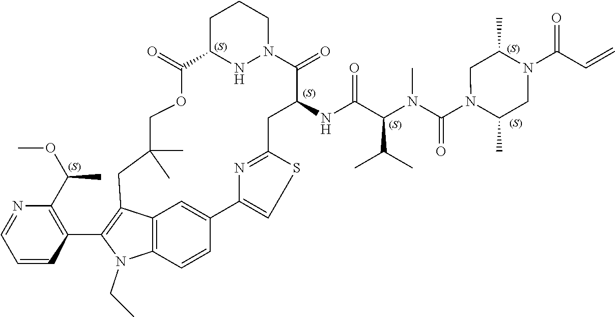 |
| BA386 | 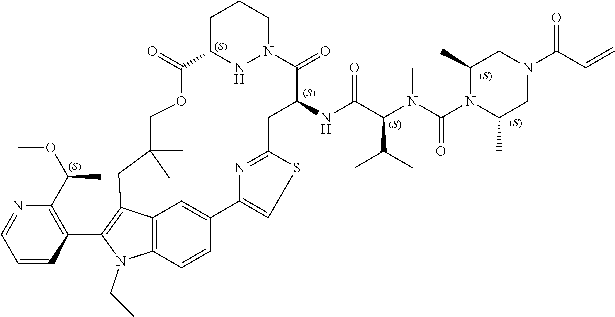 |
| BA387 | 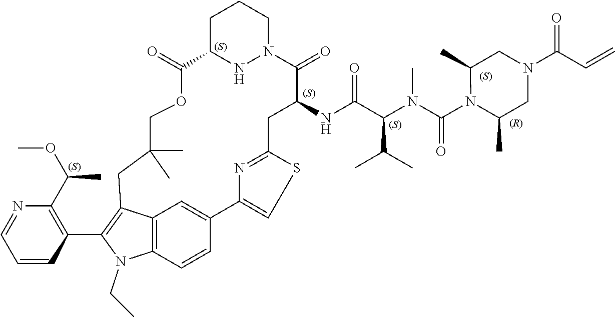 |
| BA388 | 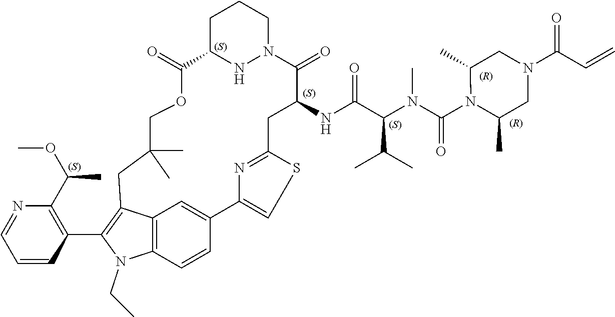 |
| BA389 | 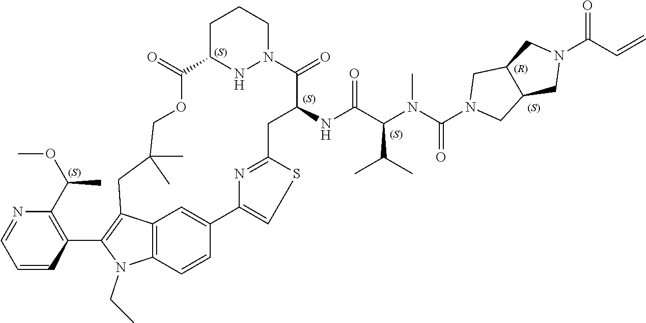 |
| BA390 | 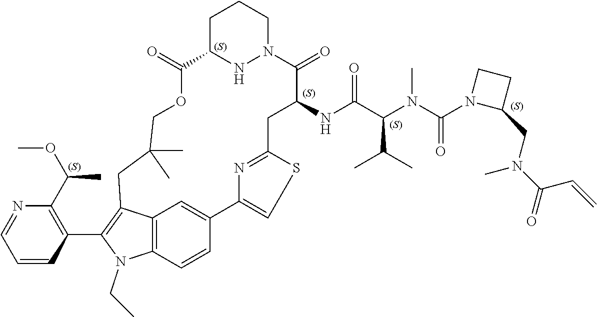 |
| BA391 | 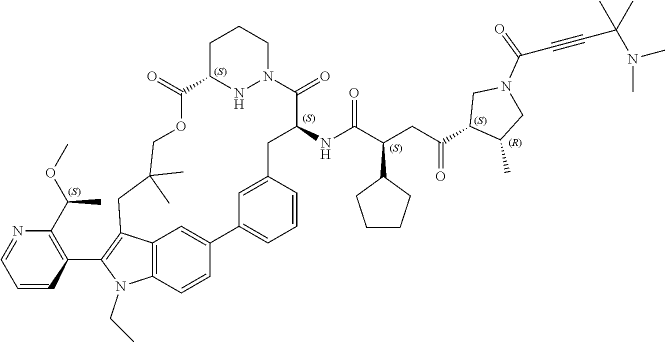 |
| BA392 | 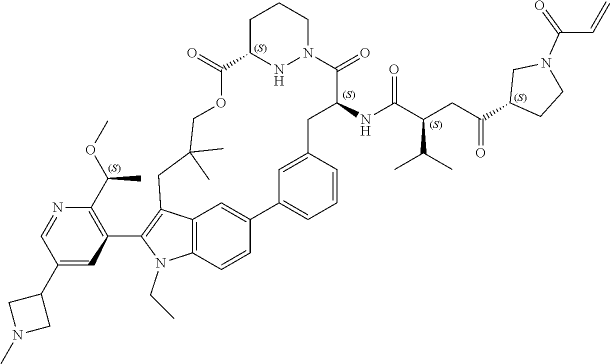 |
| BA393 | 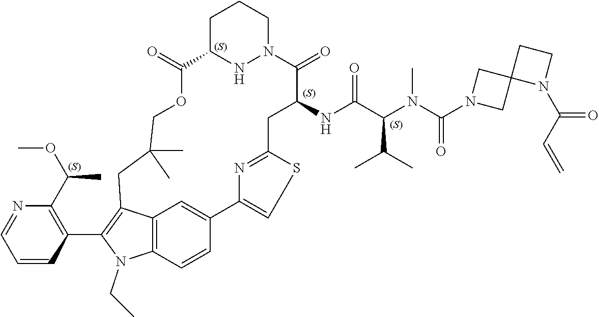 |
| BA394 | 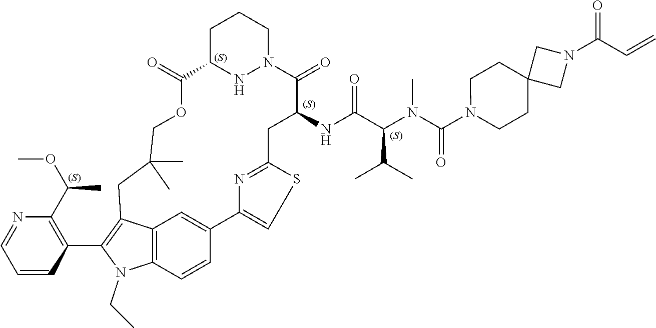 |
| BA395 | 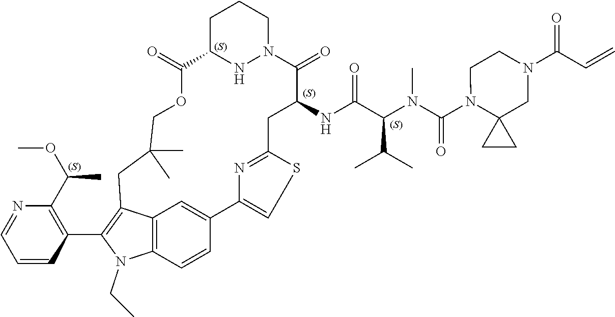 |
| BA396 | 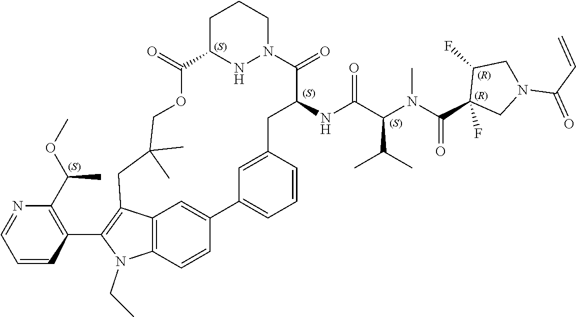 |
| BA397 | 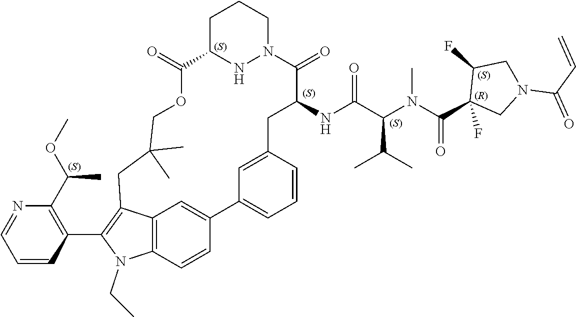 |
| BA398 | 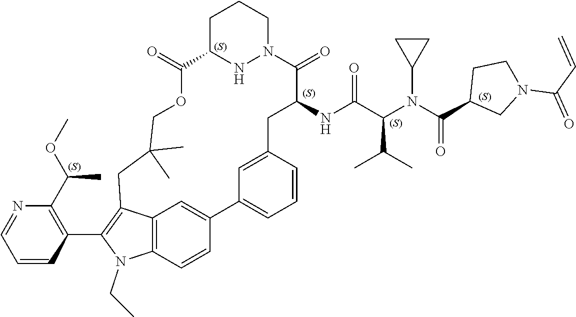 |
| BA399 | 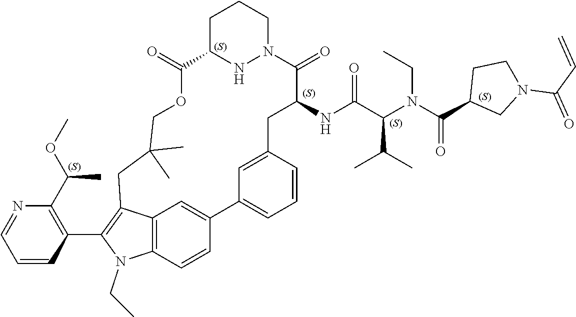 |
| BA400 | 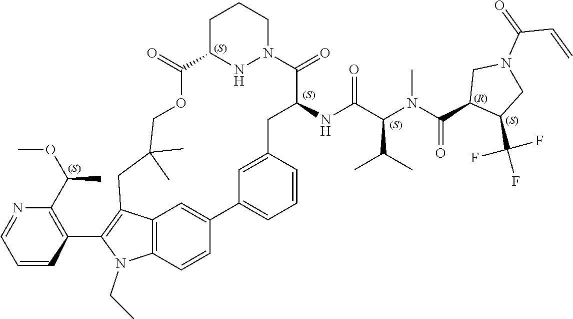 |
| BA401 | 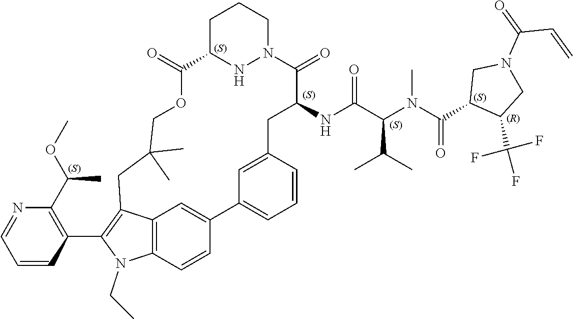 |
| BA402 | 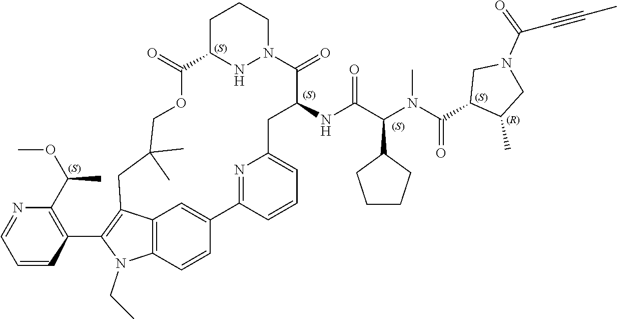 |
| BA403 | 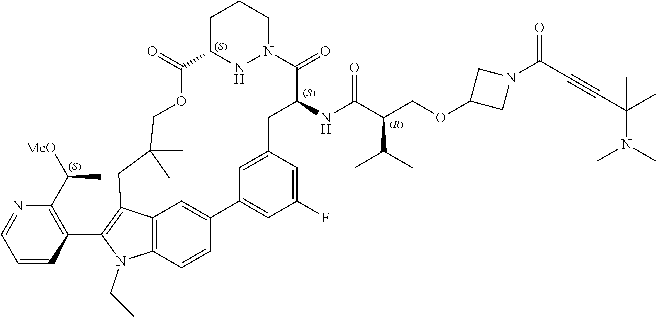 |
| BA404 | 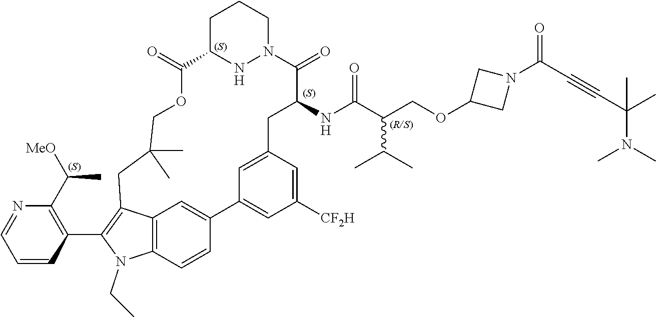 |
| BA405 | 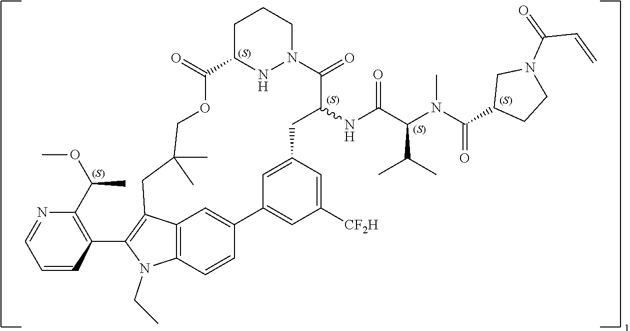 |
| BA406 | 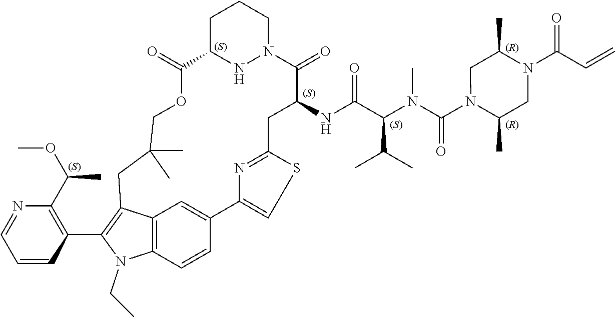 |
| BA407 | 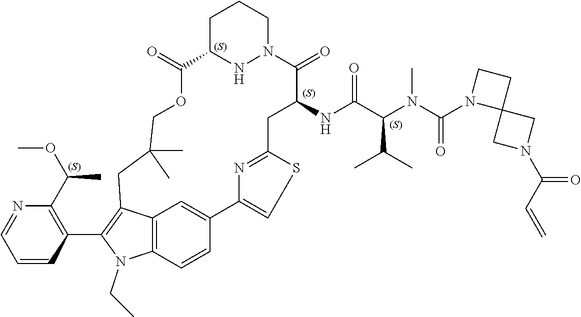 |
| BA408 | 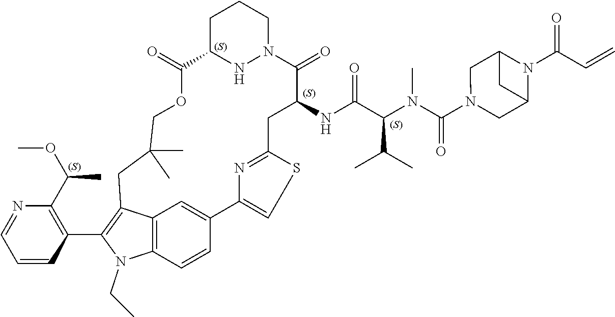 |
| BA409 | 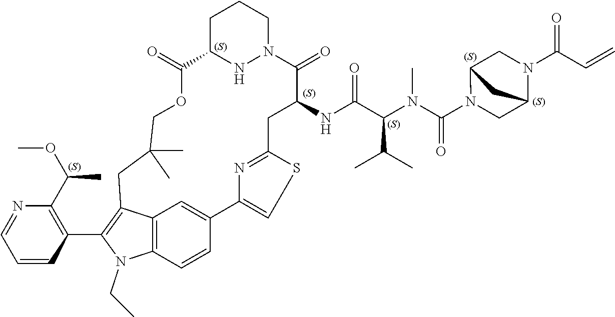 |
| BA410 | 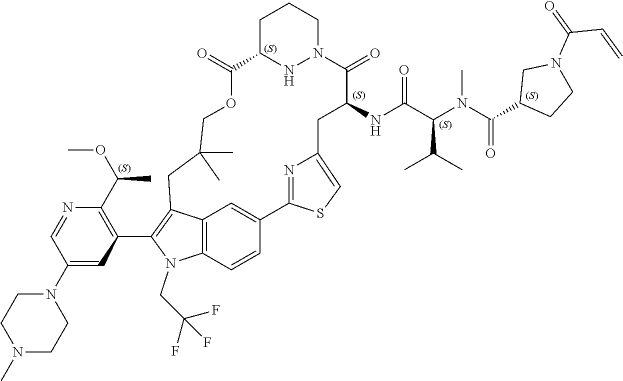 |
| BA411 | 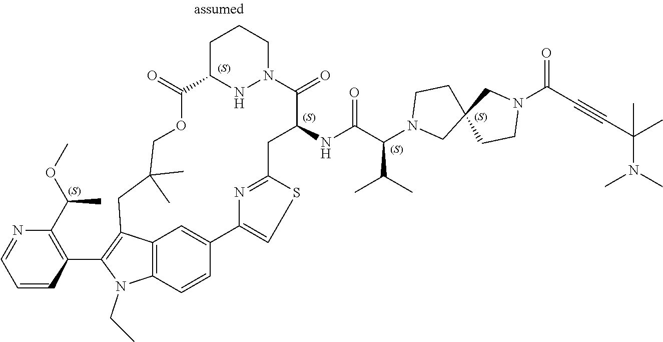 |
| BA412 | 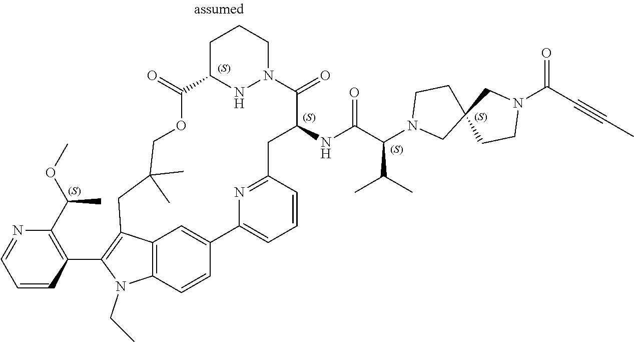 |
| BA413 | 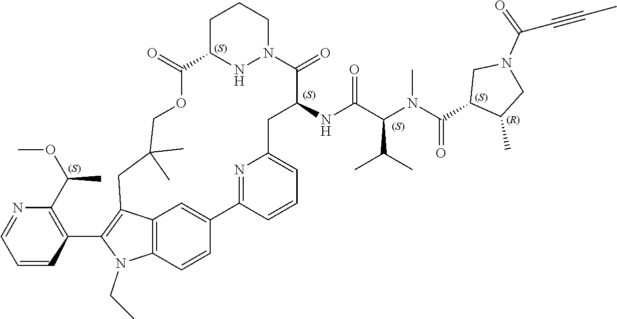 |
| BA414 | 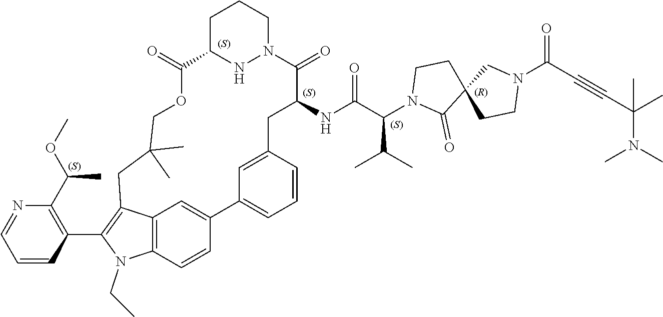 |
| BA415 | 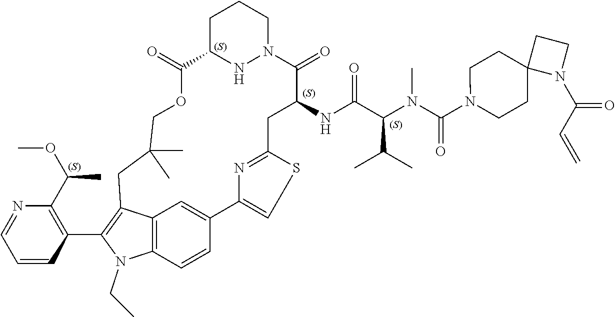 |
| BA416 | 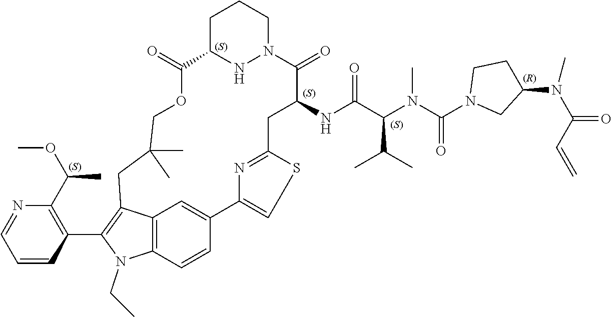 |
| BA417 | 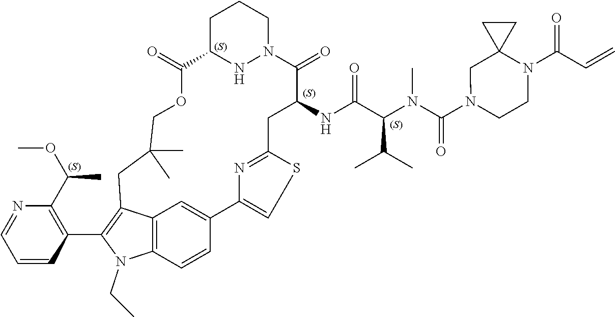 |
| BA418 | 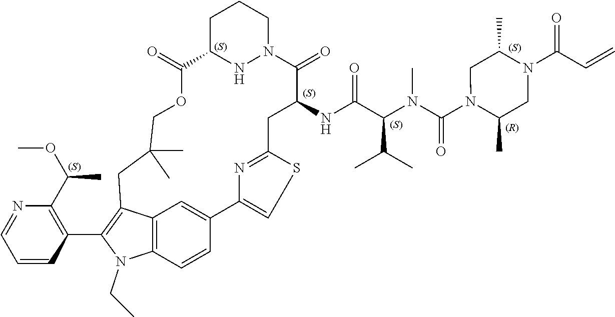 |
| BA419 | 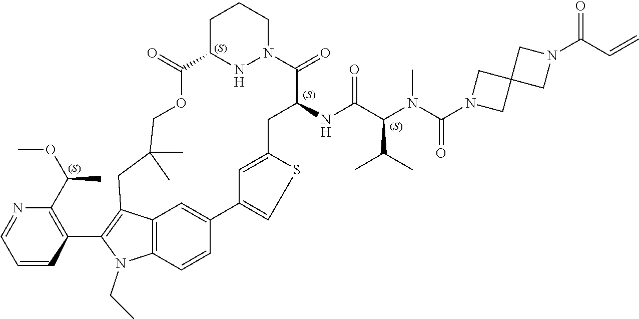 |
| BA420 | 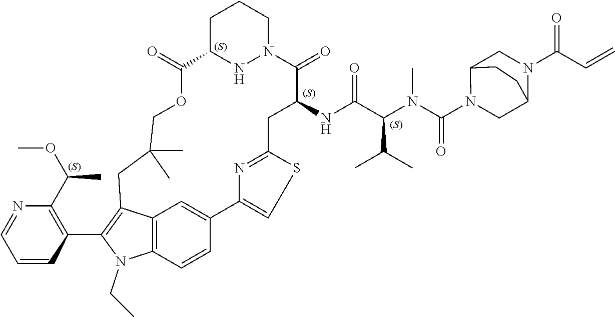 |
| BA421 | 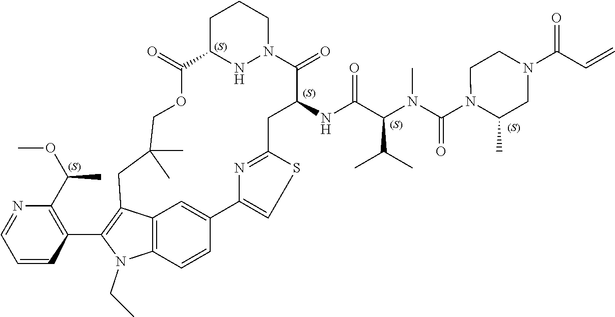 |
| BA422 | 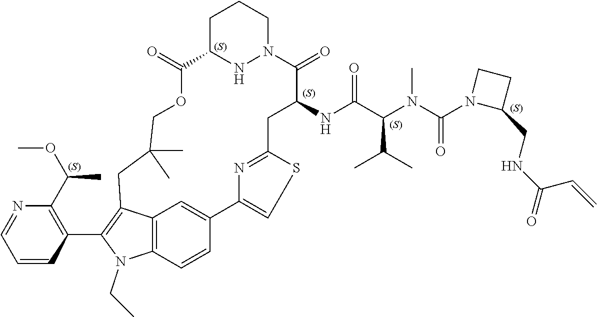 |
| BA423 | 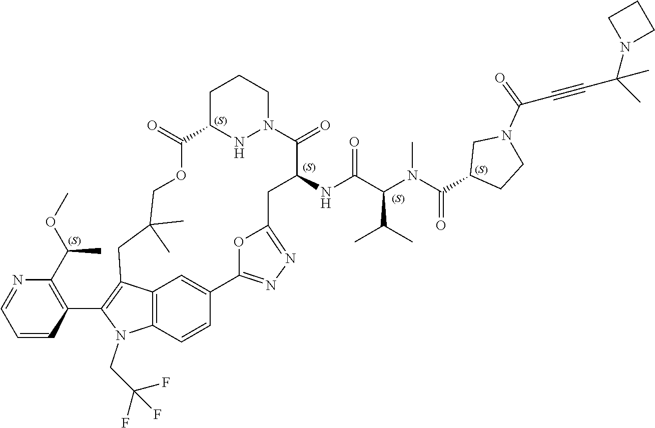 |
| BA424 | 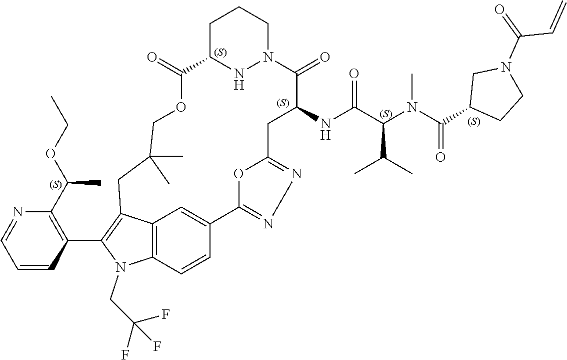 |
| BA425 | 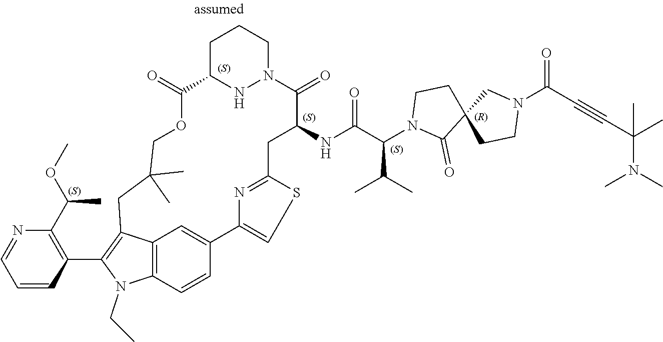 |
| BA426 | 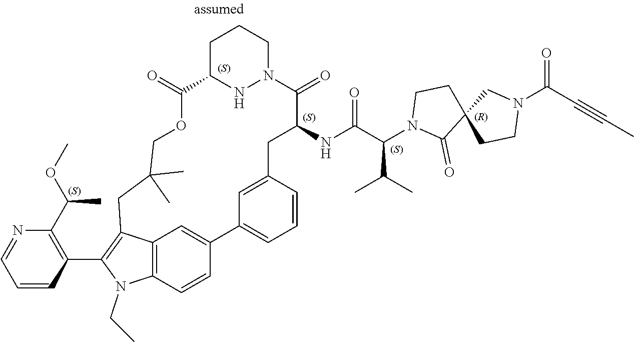 |
| BA427 | 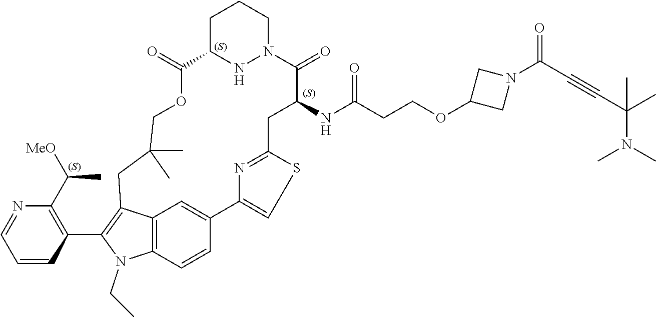 |
| BA428 | 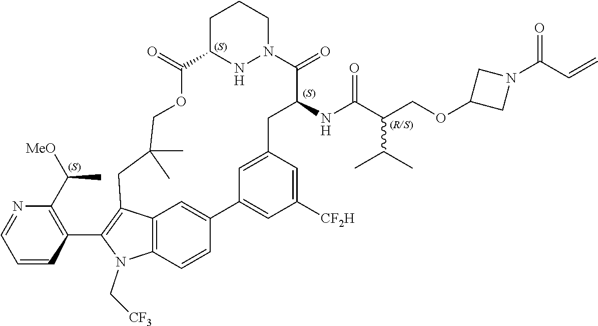 |
| BA429 | 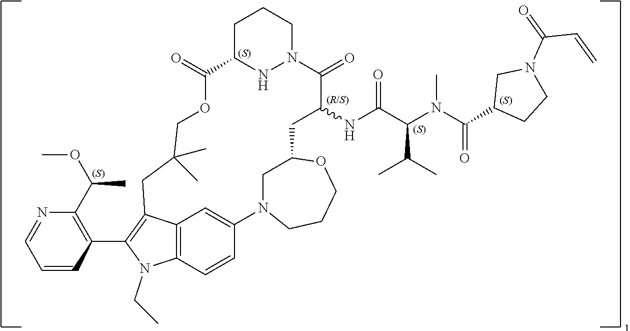 |
| BA430 | 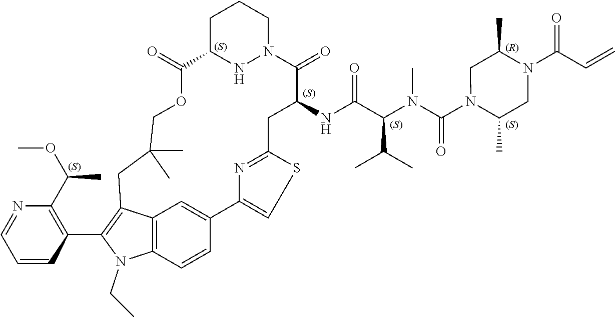 |
| BA431 | 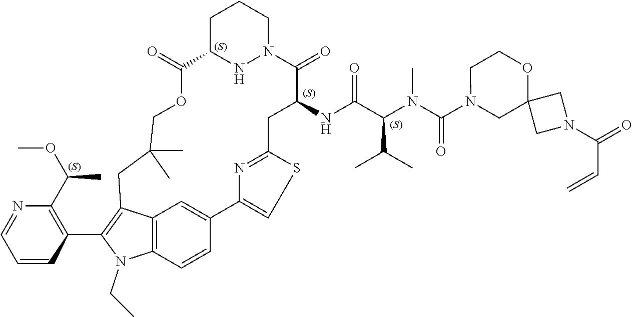 |
| BA432 | 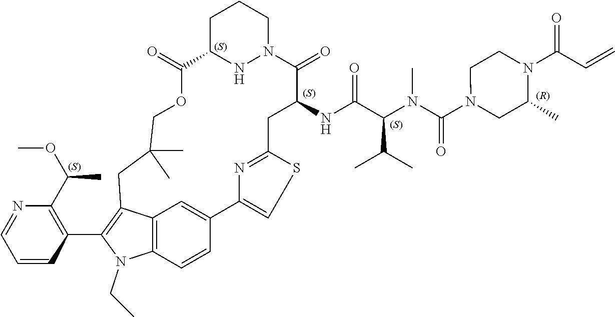 |
| BA433 | 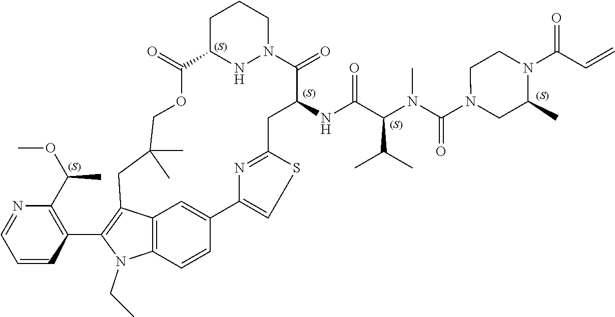 |
| BA334 | 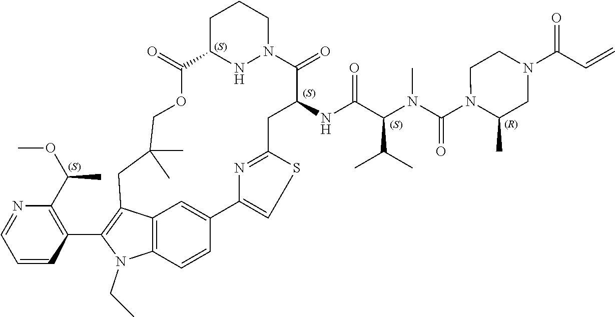 |
| BA435 | 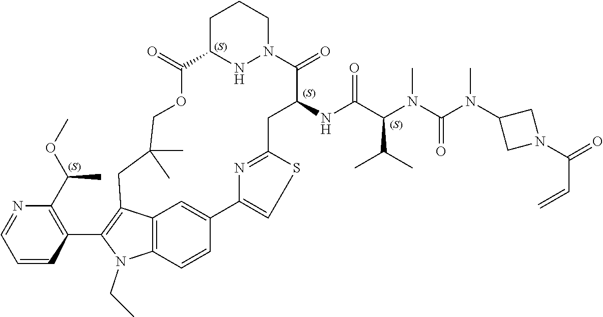 |
| BA436 | 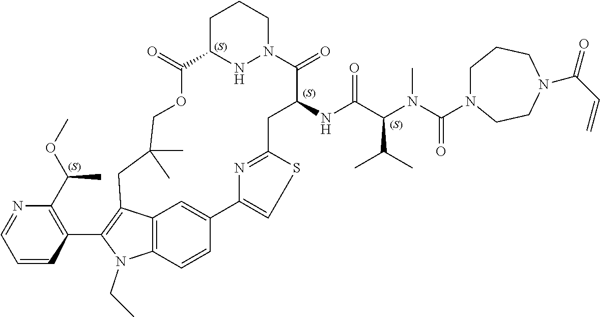 |
| BA437 | 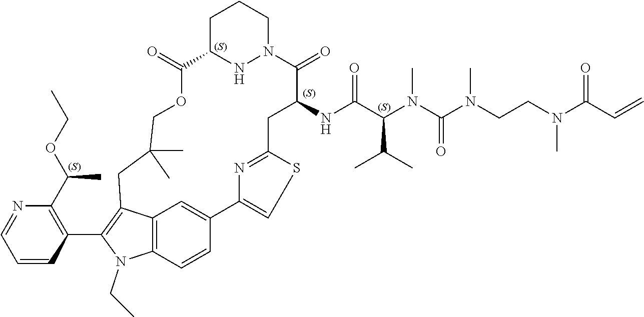 |
| BA438 | 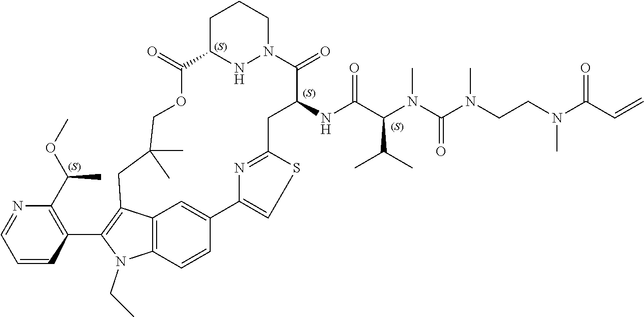 |
| BA439 | 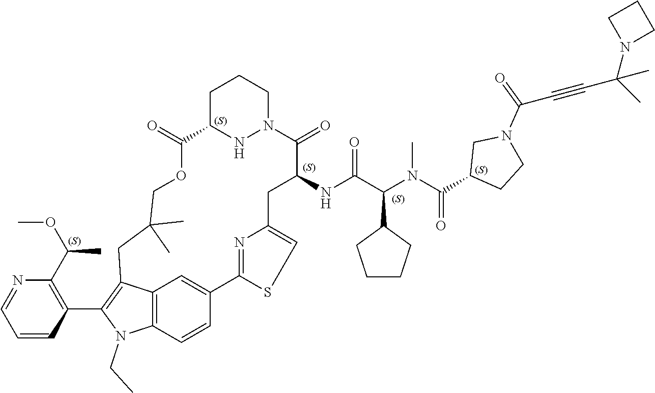 |
| BA440 | 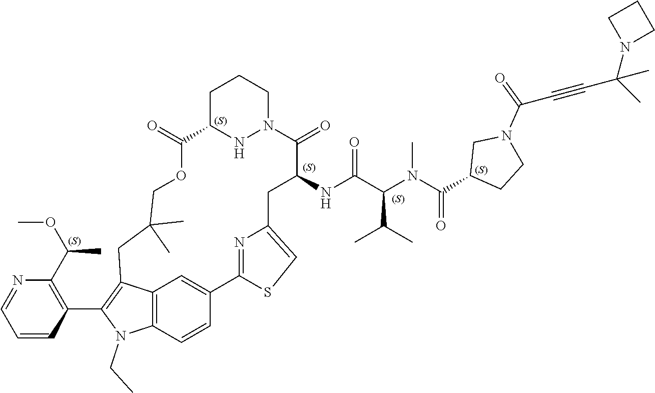 |
| BA441 | 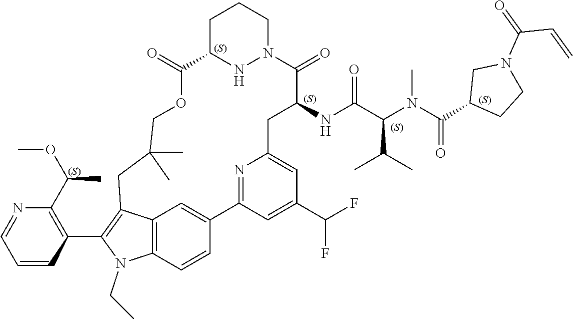 |
| BA442 | 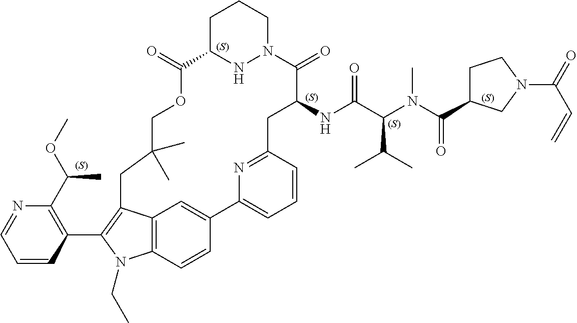 |
| BA443 | 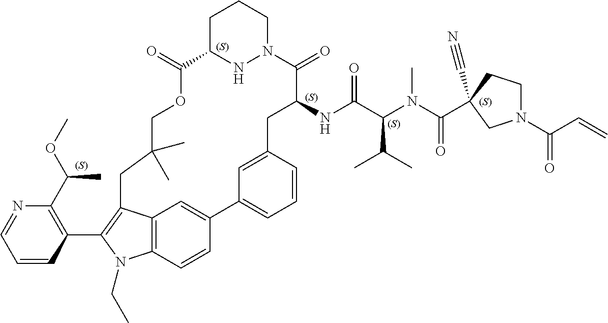 |
| BA444 | 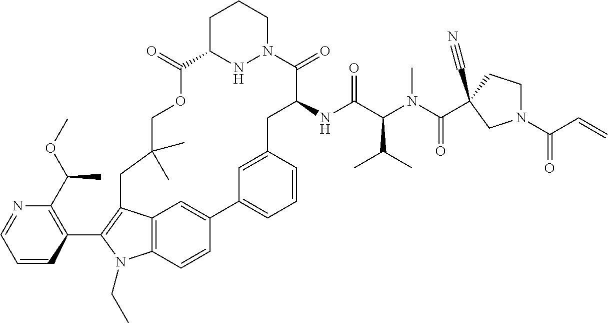 |
| BA445 | 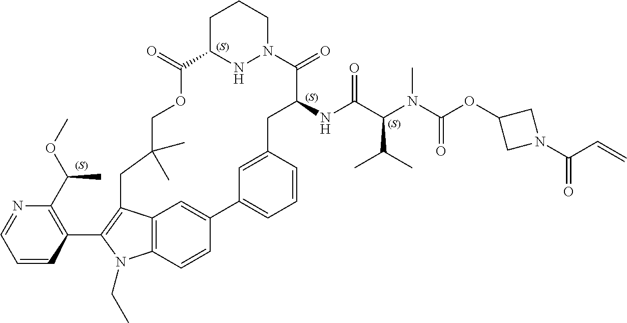 |
| BA446 | 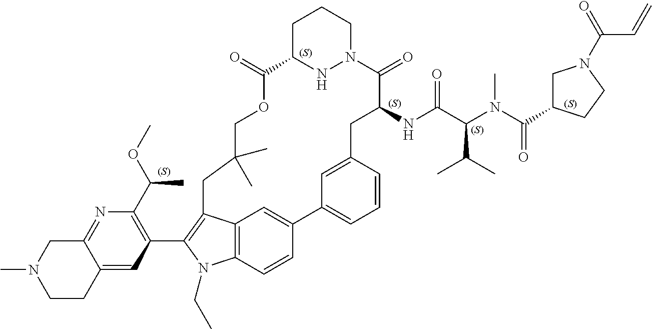 |
| BA447 | 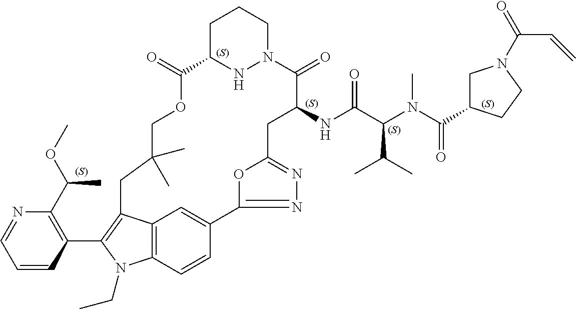 |
| BA448 | 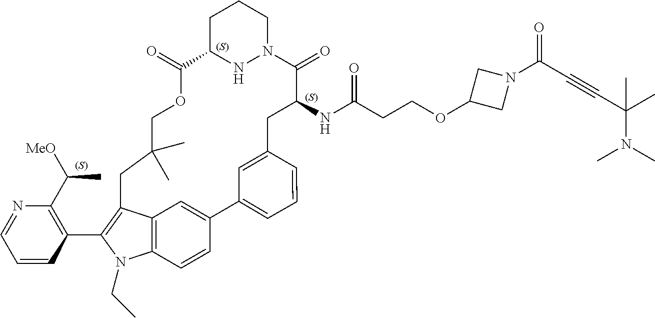 |
| BA449 | 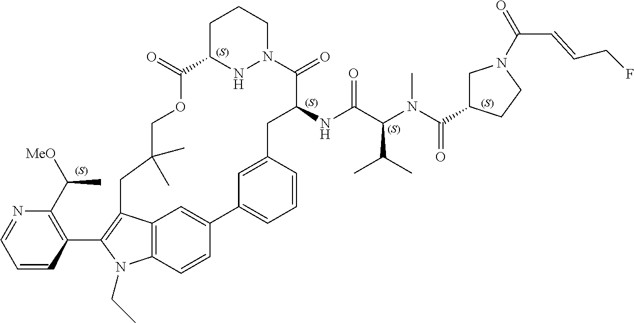 |
| BA450 | 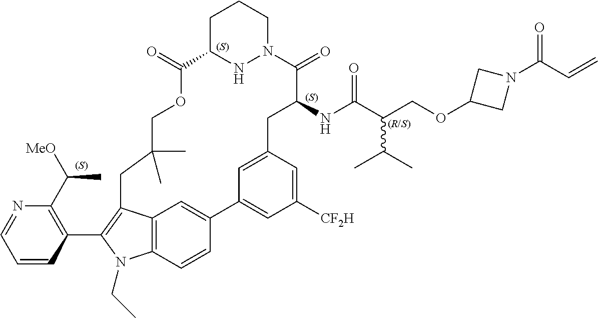 |
| BA451 | 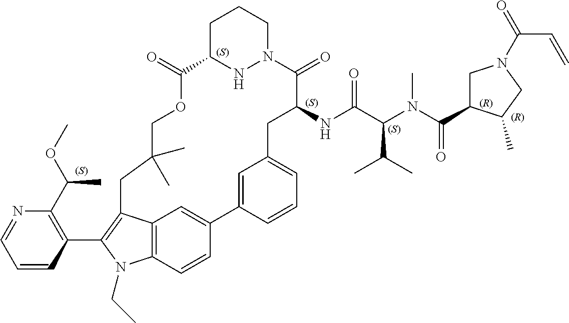 |
| BA452 | 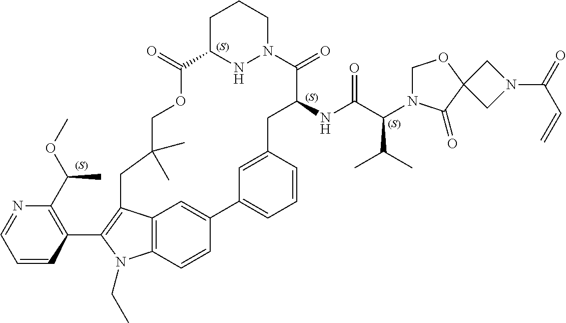 |
| BA453 | 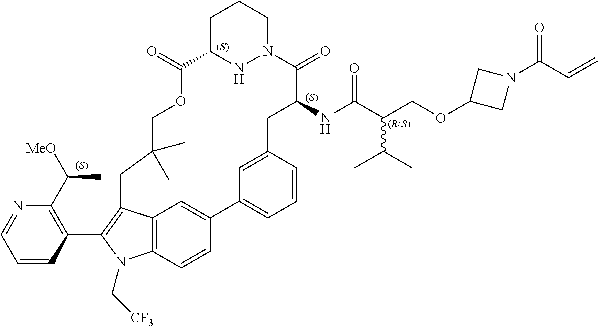 |
| BA454 | 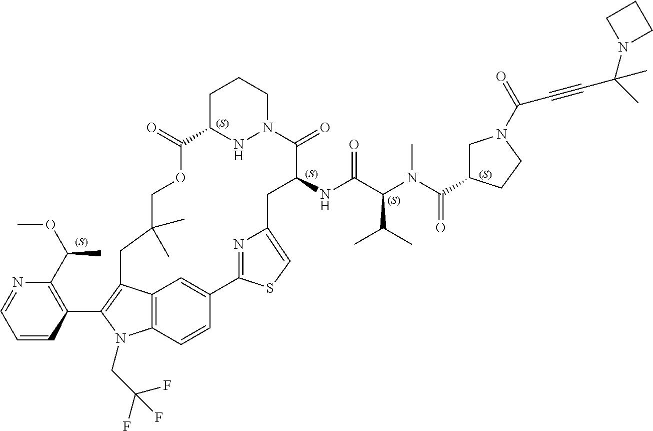 |
| BA455 | 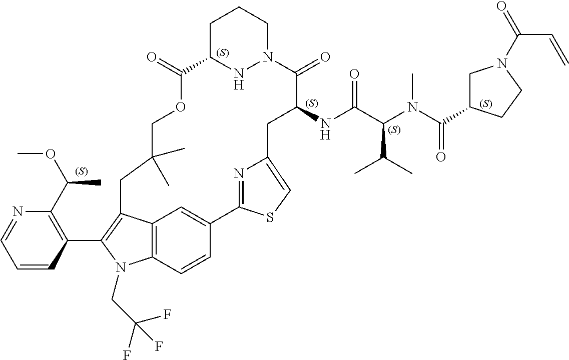 |
| BA456 | 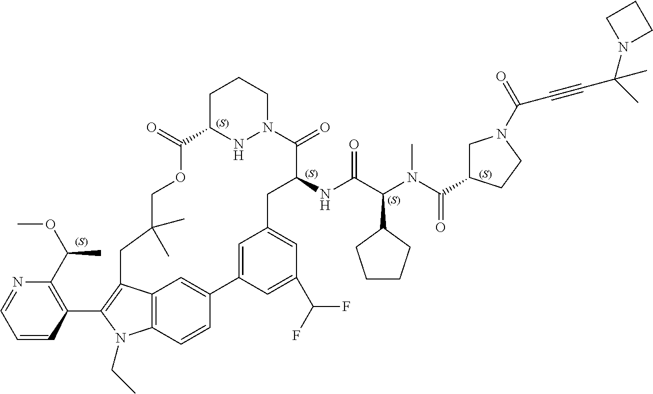 |
| BA457 | 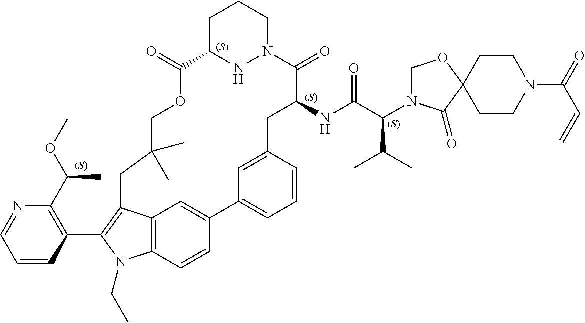 |
| BA458 | 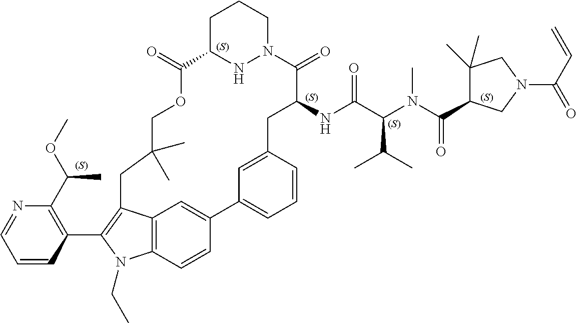 |
| BA459 | 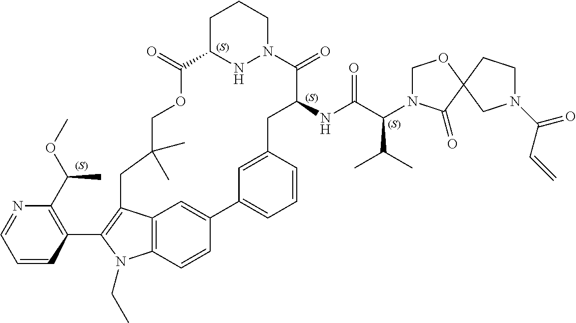 |
| BA460 | 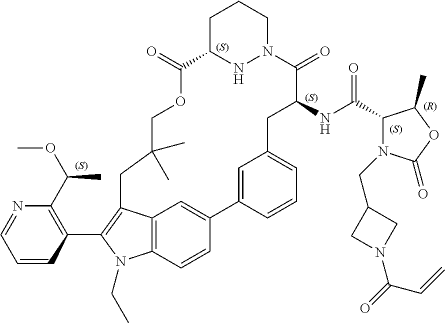 |
| BA461 | 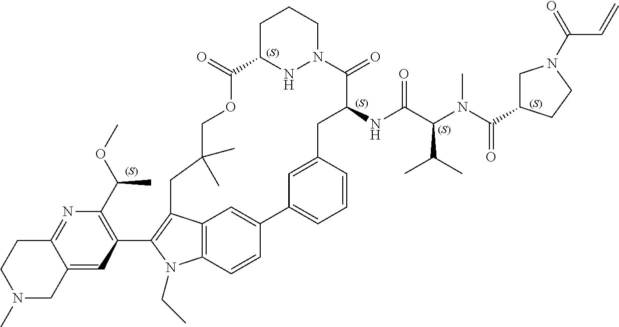 |
| BA462 | 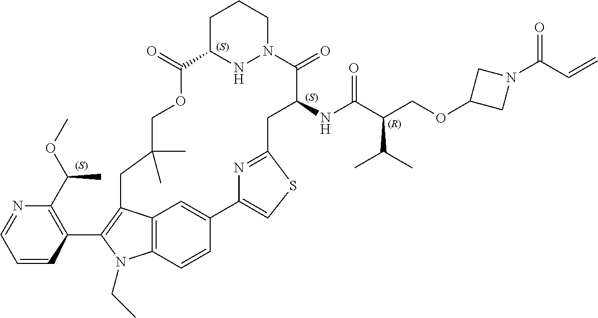 |
| BA463 | 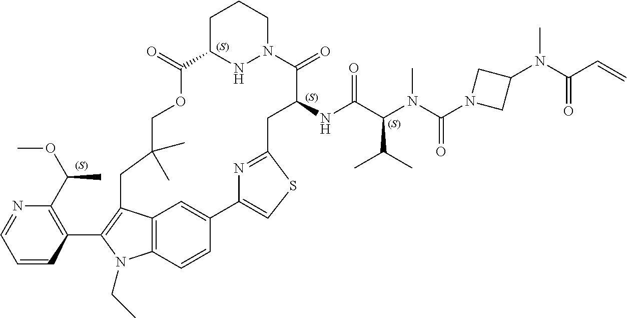 |
| BA464 | 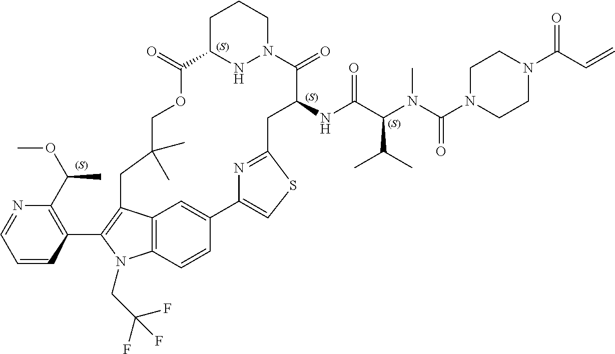 |
| BA465 | 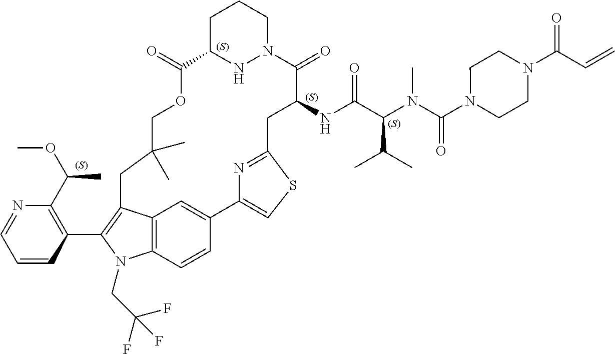 |
| BA466 | 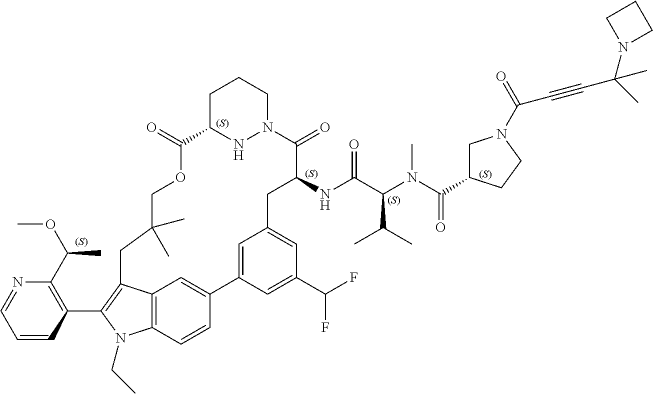 |
| BA467 | 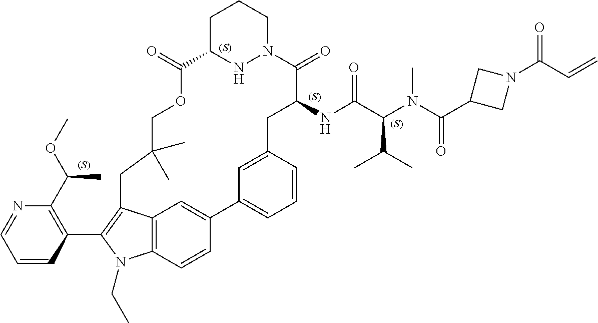 |
| BA468 | 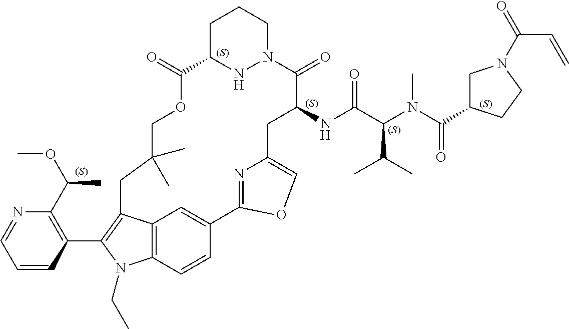 |
| BA469 | 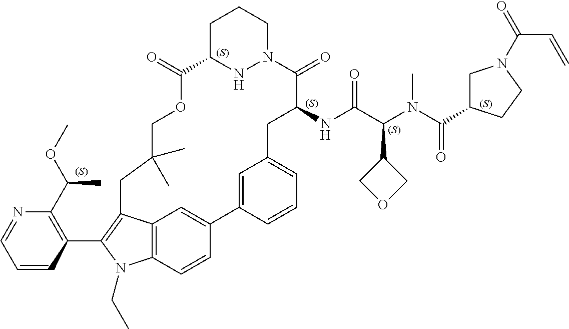 |
| BA470 | 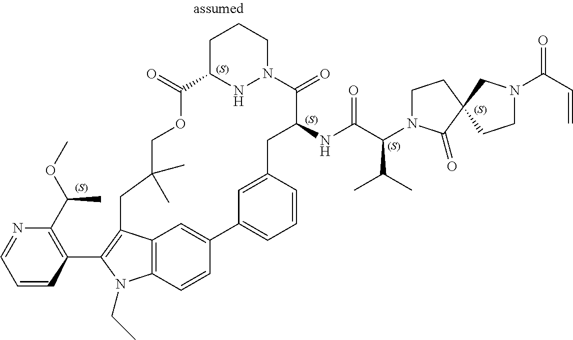 |
| BA471 | 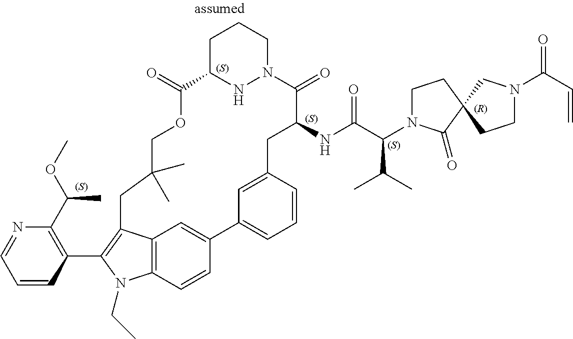 |
| BA472 | 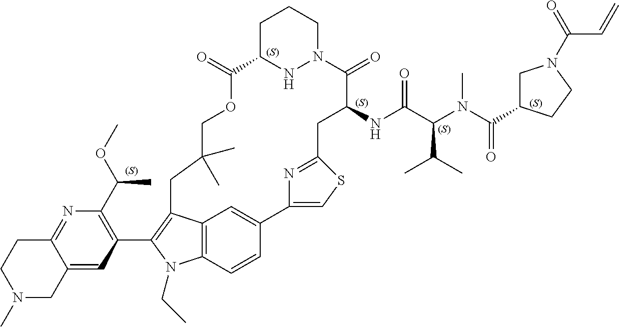 |
| BA473 | 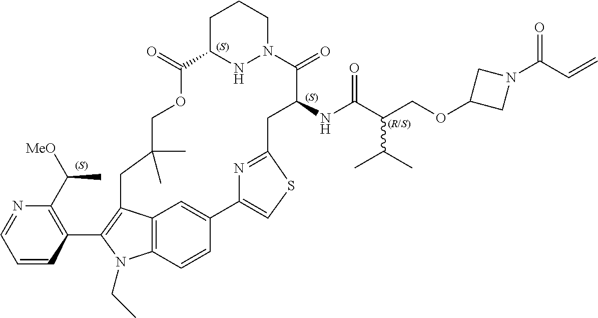 |
| BA474 | 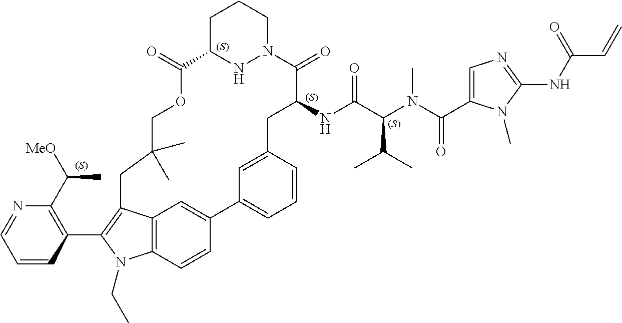 |
| BA475 | 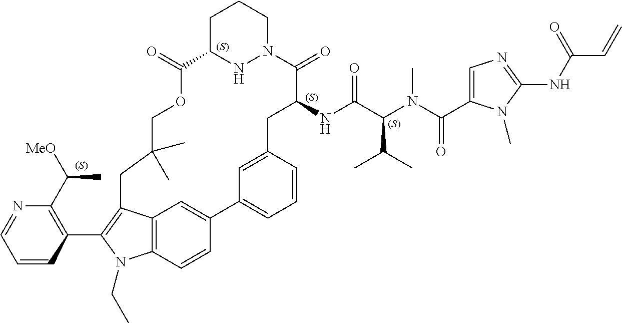 |
| BA476 | 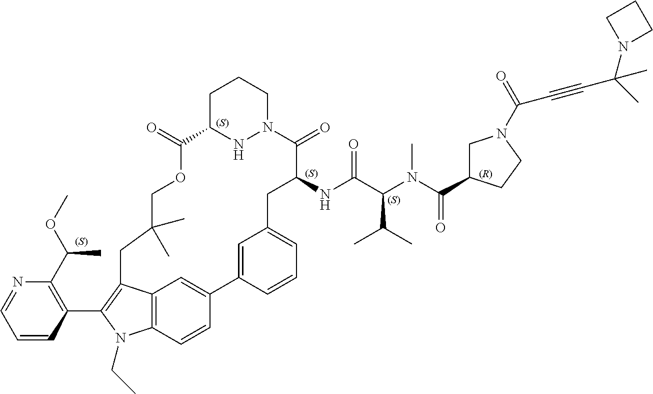 |
| BA477 | 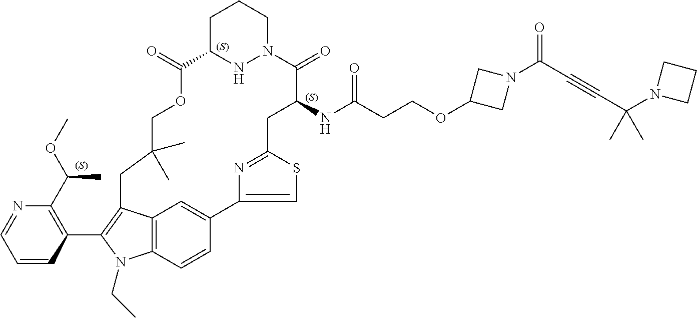 |
| BA478 | 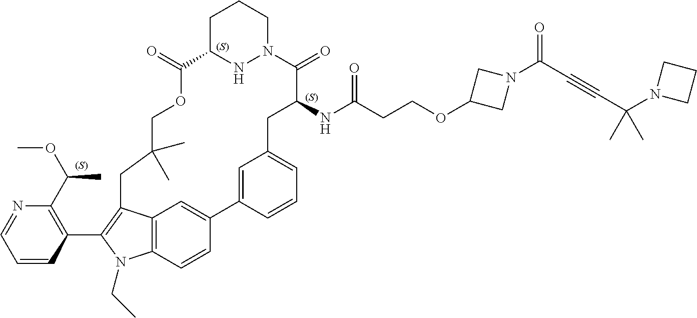 |
| BA479 | 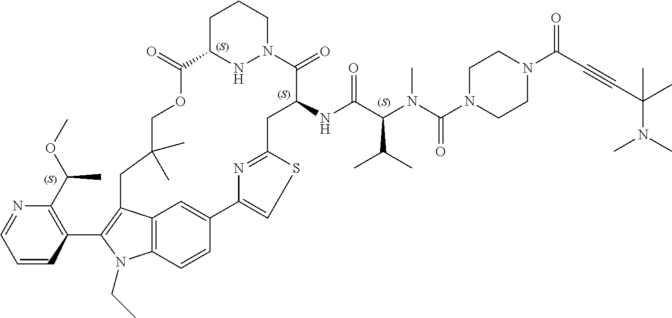 |
| BA480 | 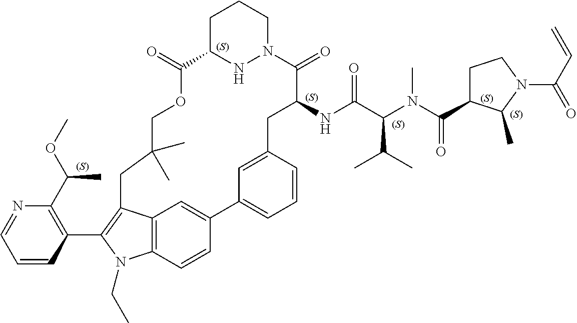 |
| BA481 | 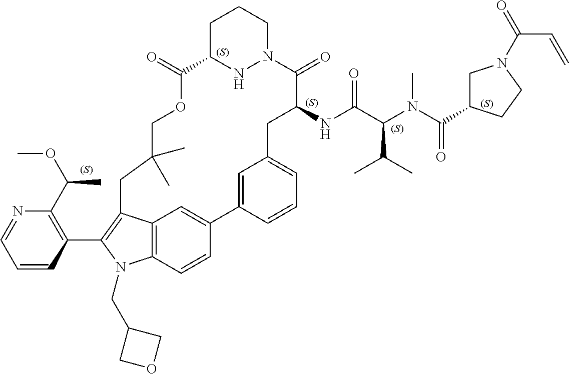 |
| BA482 | 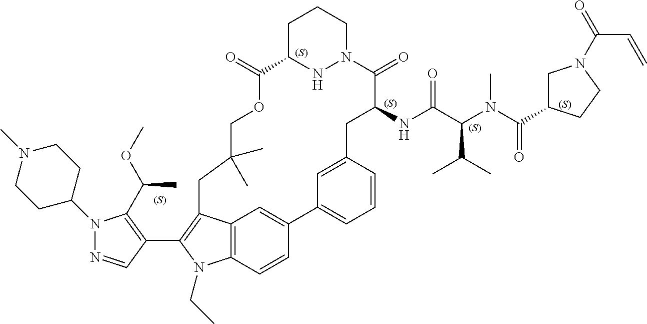 |
| BA483 | 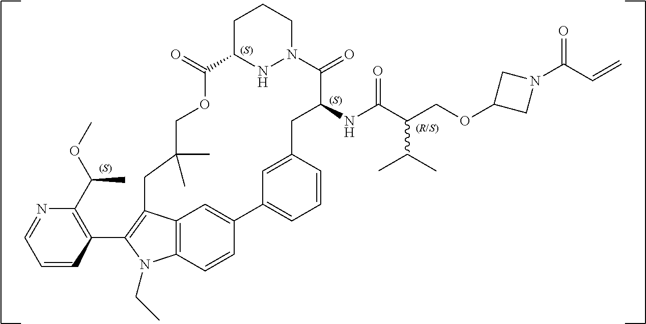 |
| BA484 | 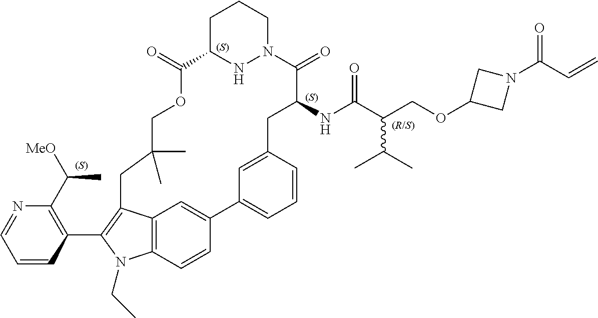 |
| BA485 | 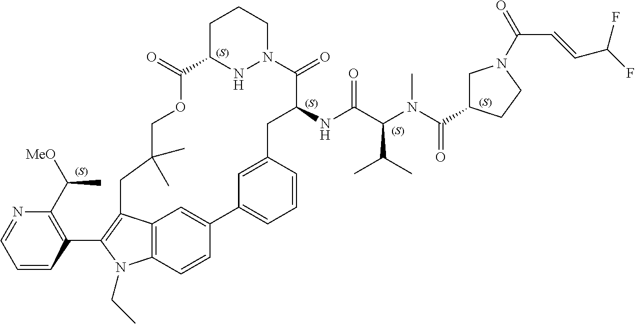 |
| BA486 | 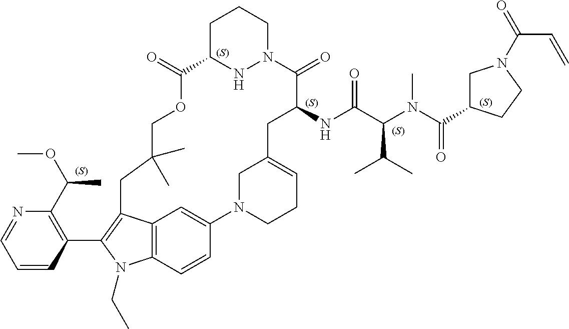 |
| BA487 | 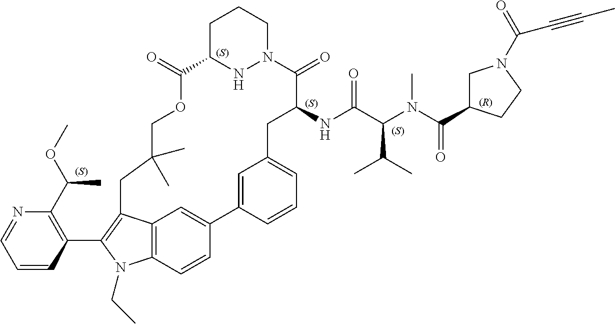 |
| BA488 | 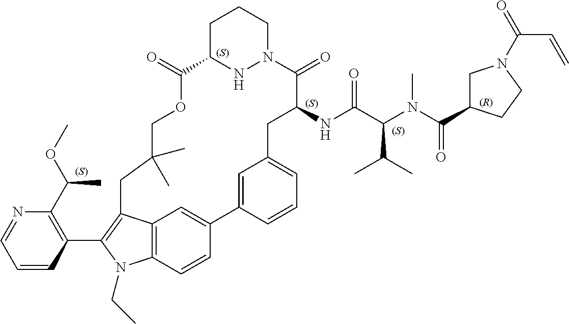 |
| BA489 | 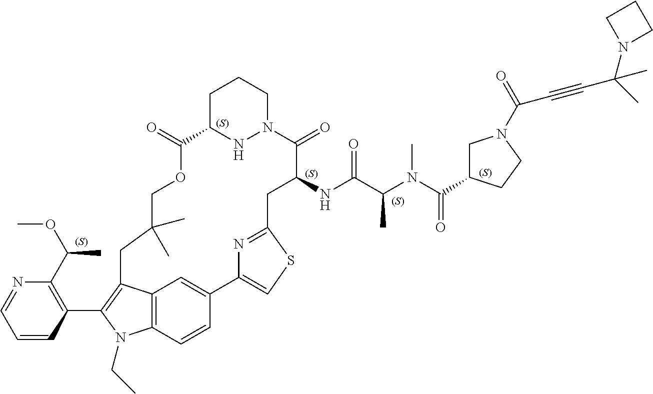 |
| BA490 | 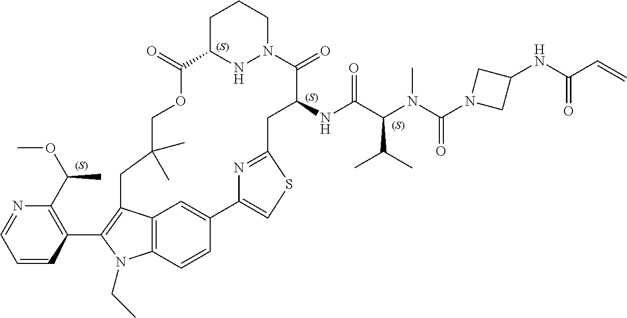 |
| BA491 | 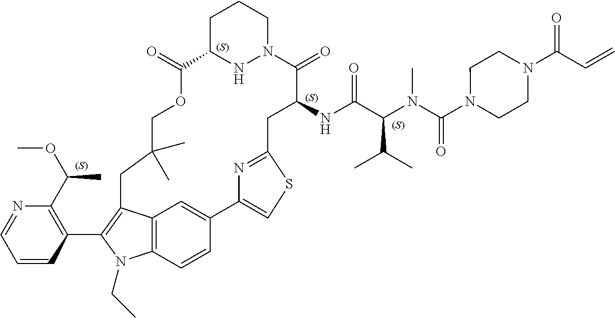 |
| BA492 | 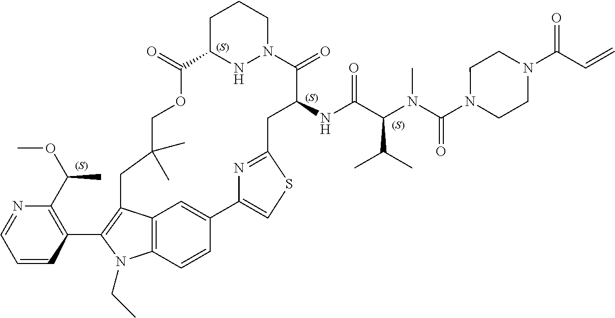 |
| BA493 | 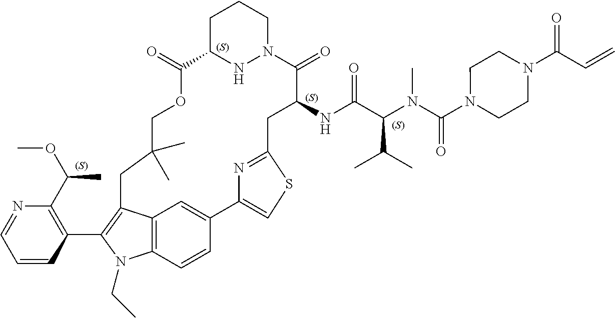 |
| BA494 | 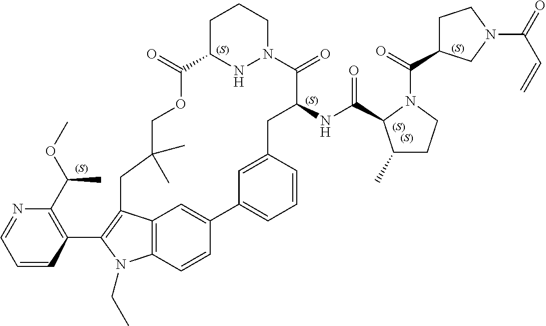 |
| BA495 | 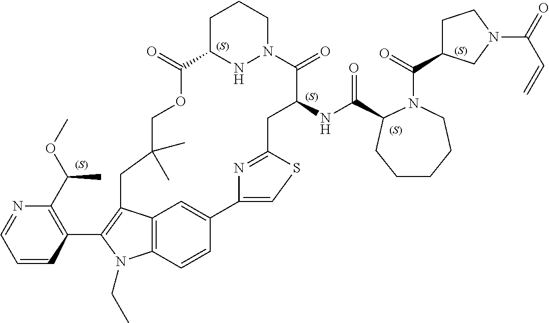 |
| BA496 | 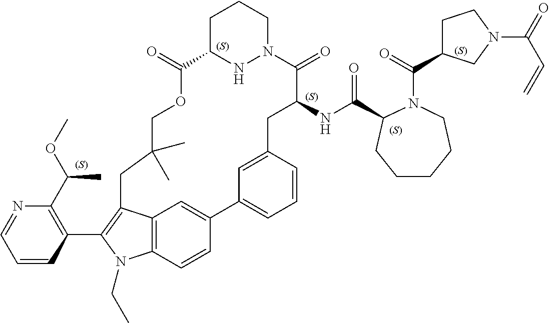 |
| BA497 | 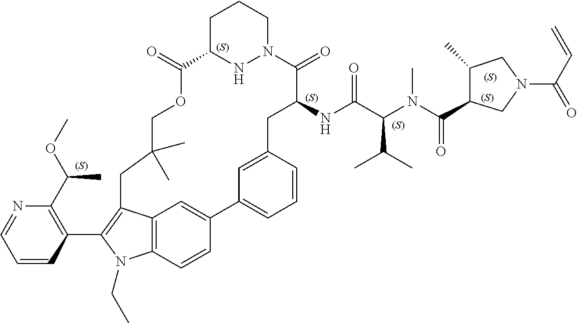 |
| BA498 | 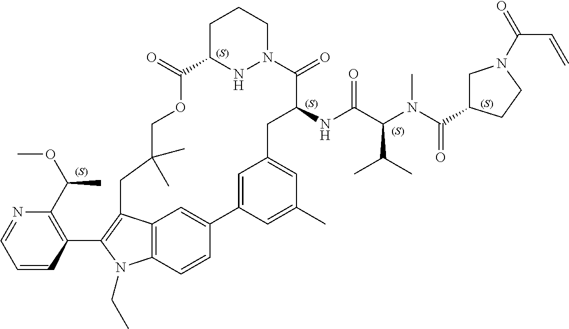 |
| BA499 | 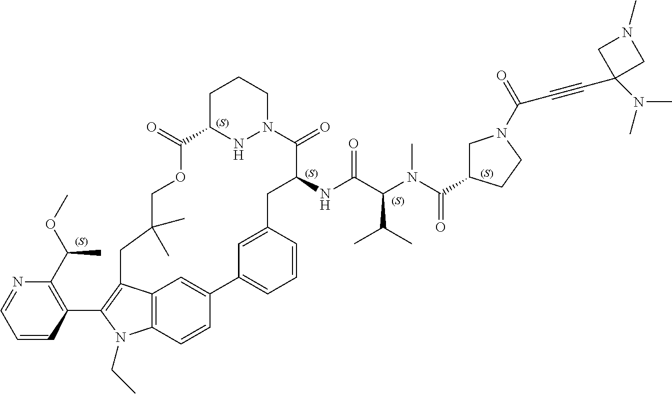 |
| BA500 | 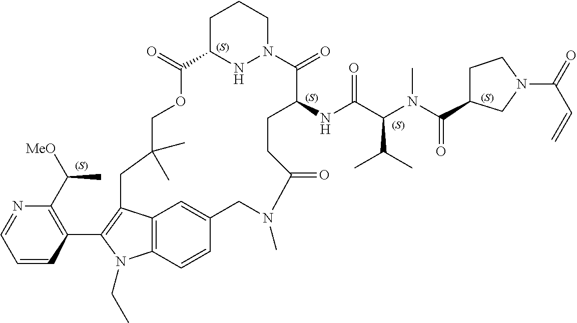 |
| BA501 | 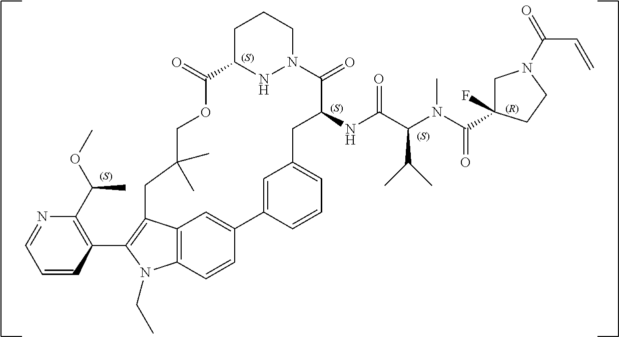 |
| BA502 | 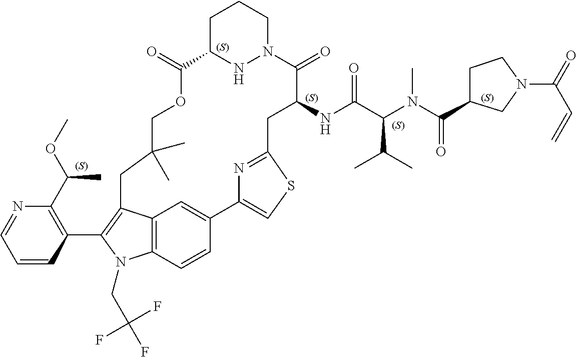 |
| BA503 | 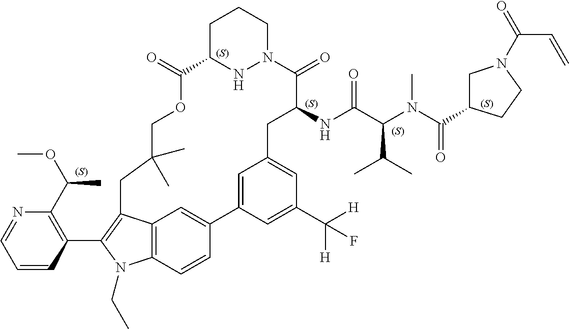 |
| BA504 | 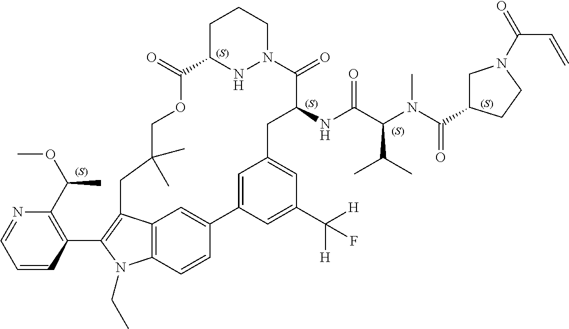 |
| BA505 | 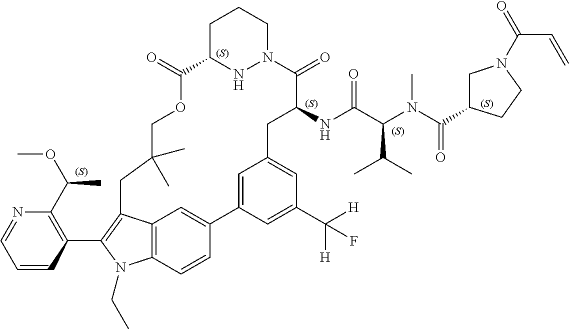 |
| BA506 | 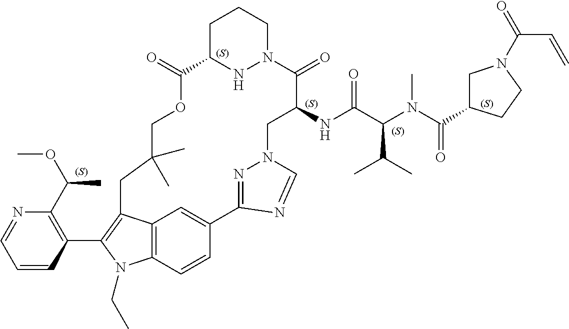 |
| BA507 | 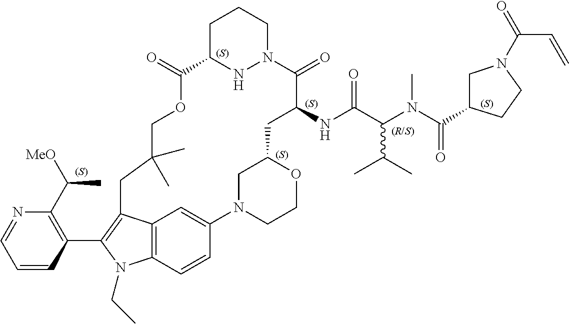 |
| BA508 | 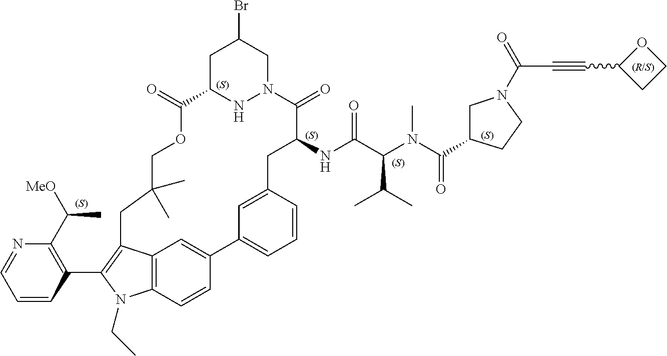 |
| BA509 | 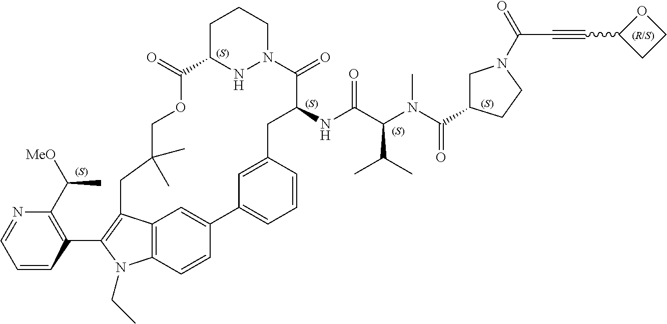 |
| BA510 | 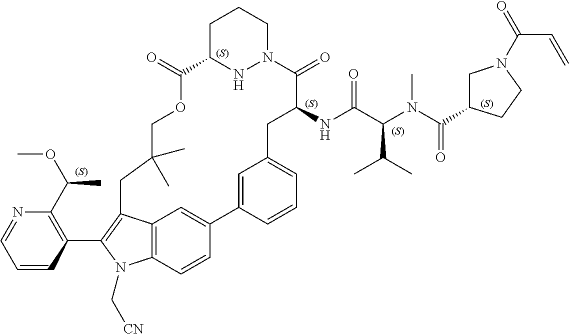 |
| BA511 | 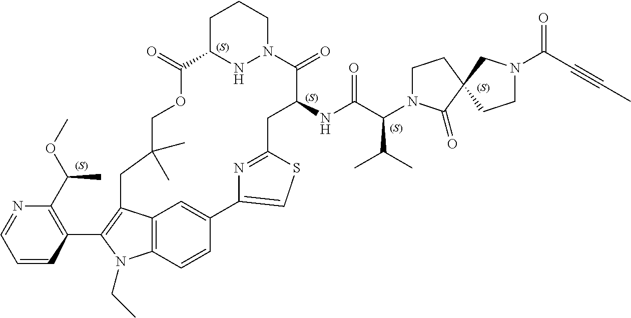 |
| BA512 | 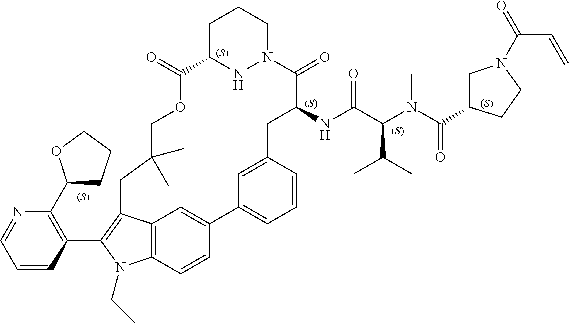 |
| BA513 | 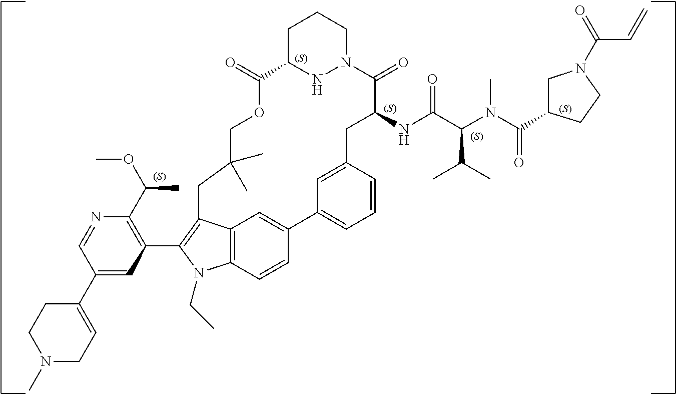 |
| BA514 | 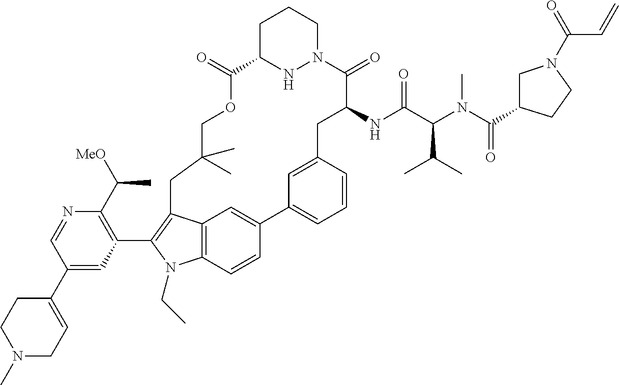 |
| BA515 | 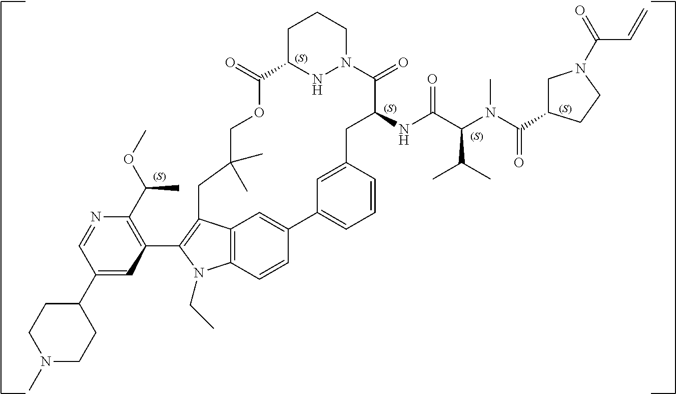 |
| BA516 | 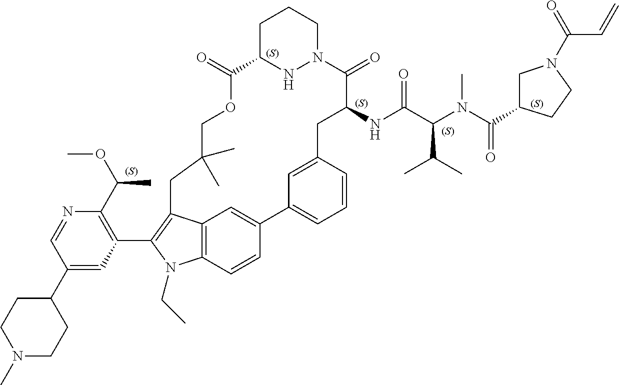 |
| BA517 | 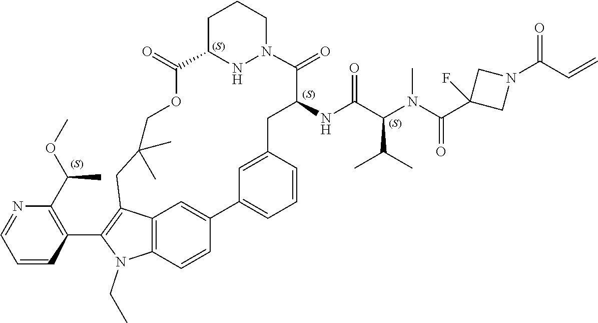 |
| BA518 | 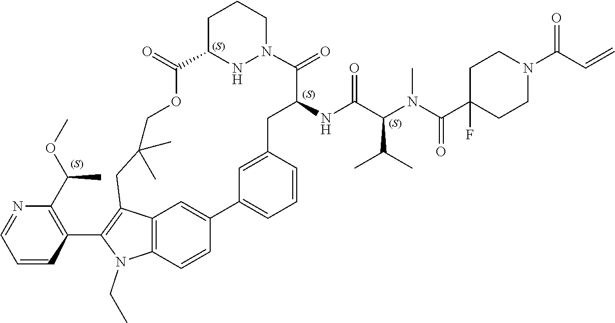 |
| BA519 | 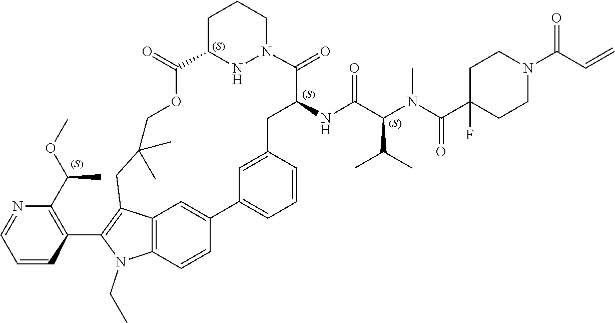 |
| BA520 | 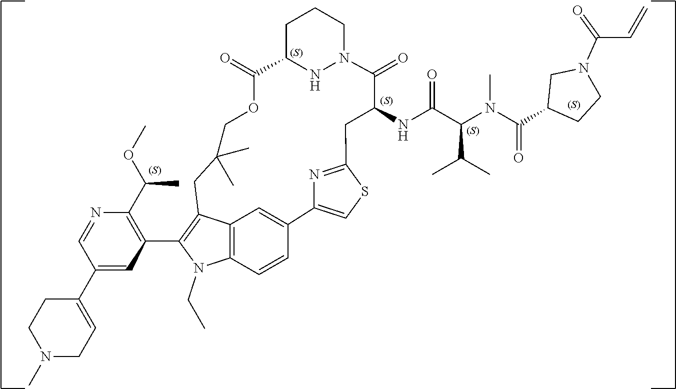 |
| BA521 | 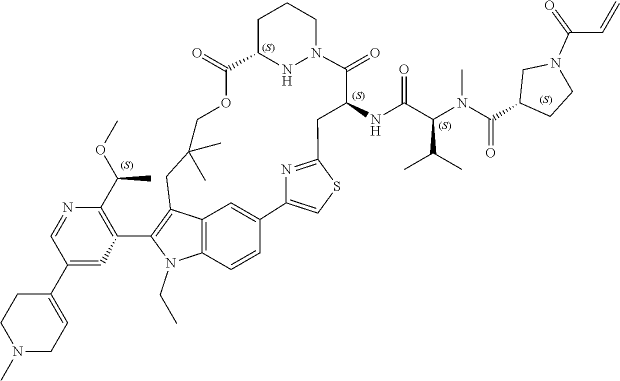 |
| BA522 | 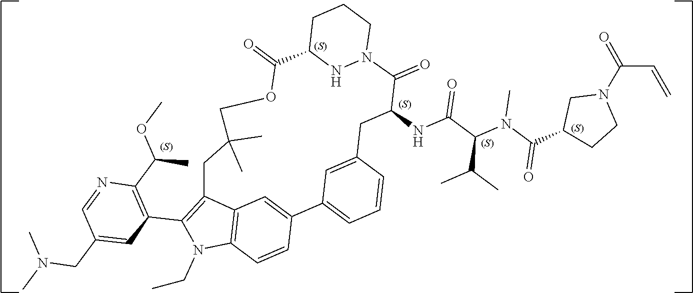 |
| BA523 | 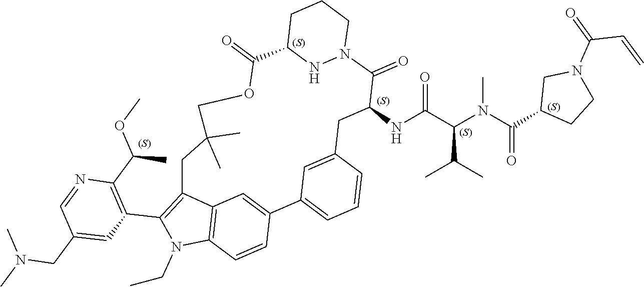 |
| BA524 | 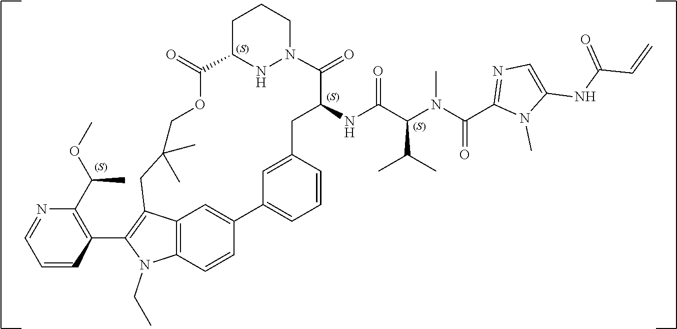 |
| BA525 | 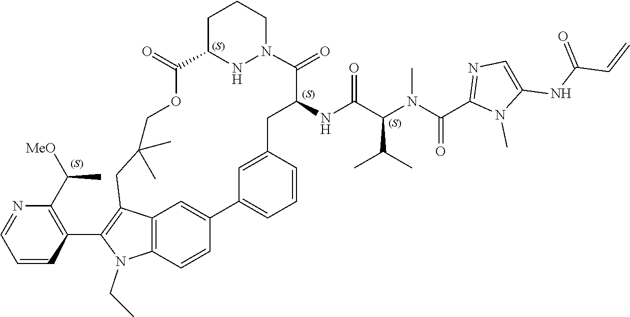 |
| BA526 | 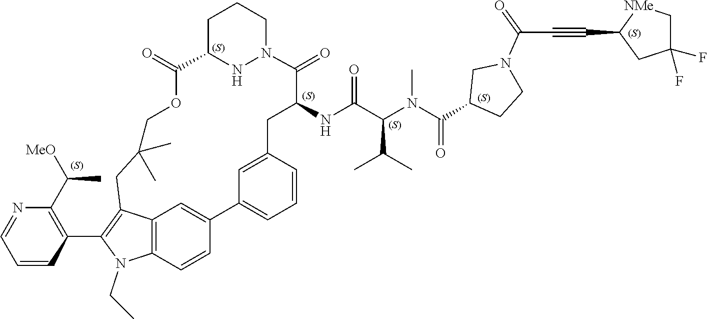 |
| BA527 | 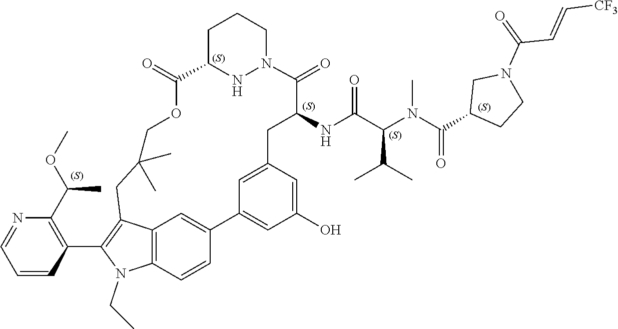 |
| BA528 | 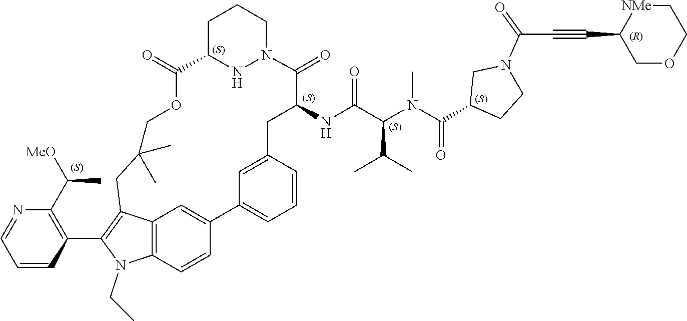 |
| BA529 | 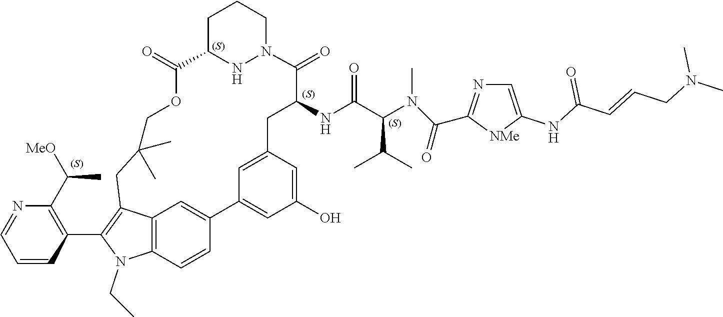 |
| BA530 | 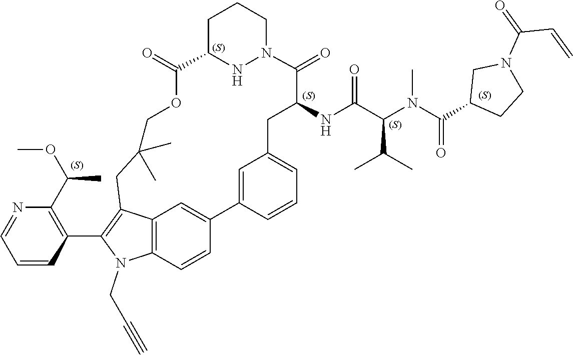 |
| BA531 | 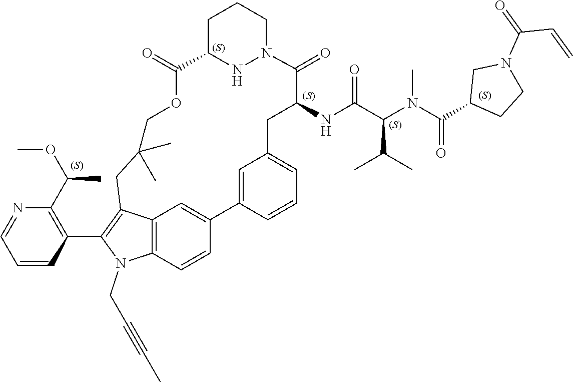 |
| BA532 | 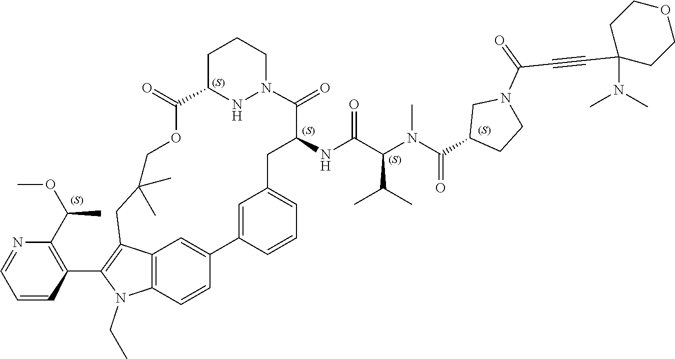 |
| BA533 | 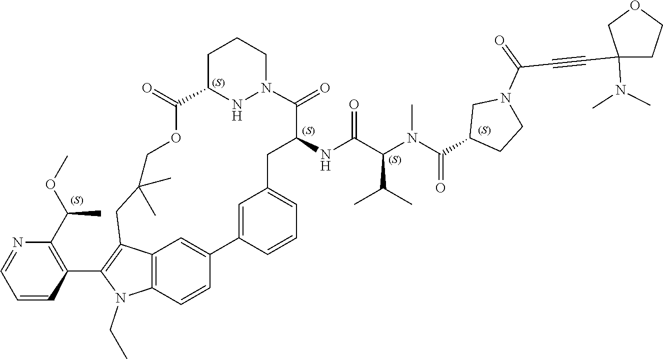 |
| BA534 | 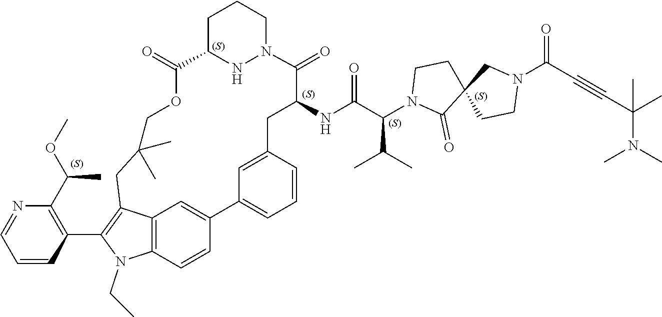 |
| BA535 | 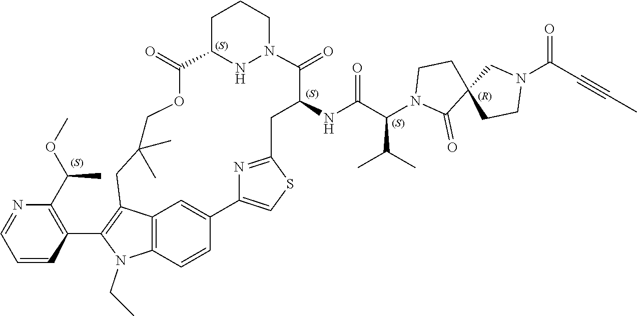 |
| BA536 | 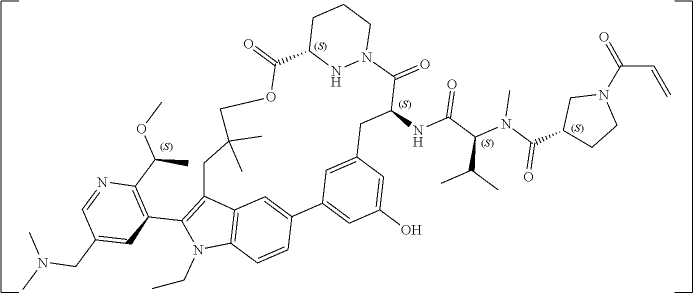 |
| BA537 | 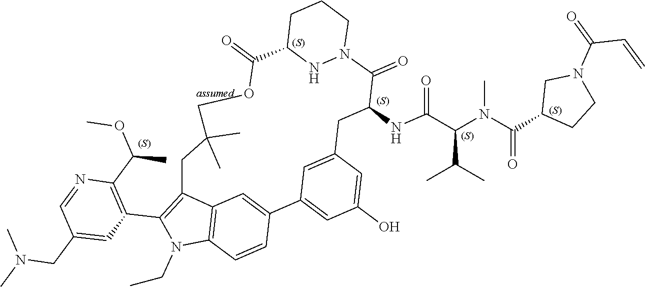 |
| BA538 | 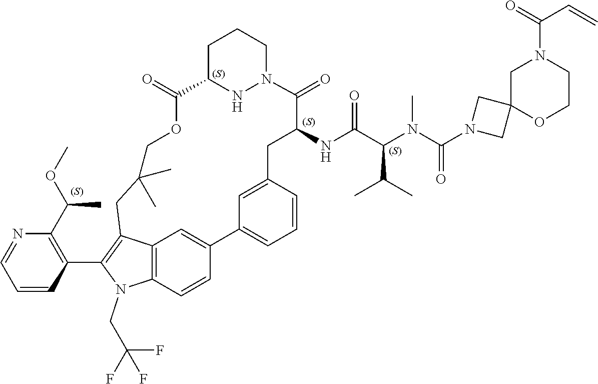 |
| BA539 | 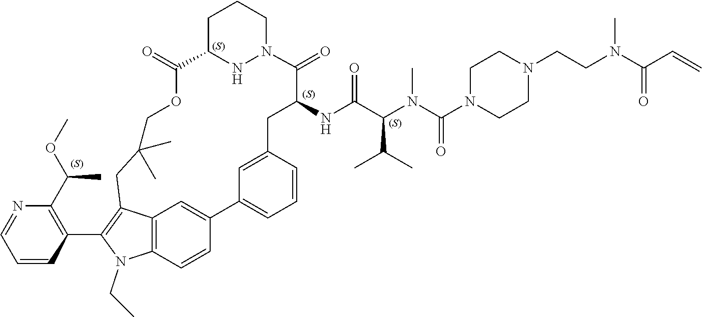 |
| BA540 | 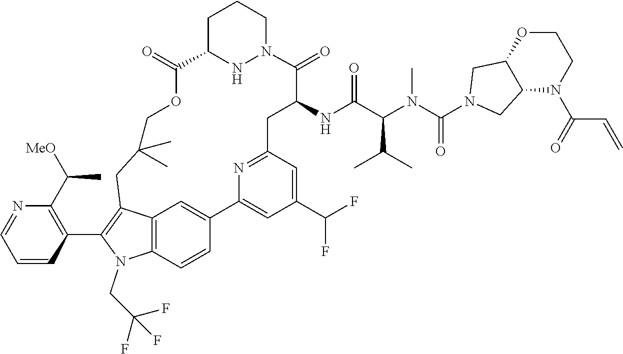 |
| BA541 | 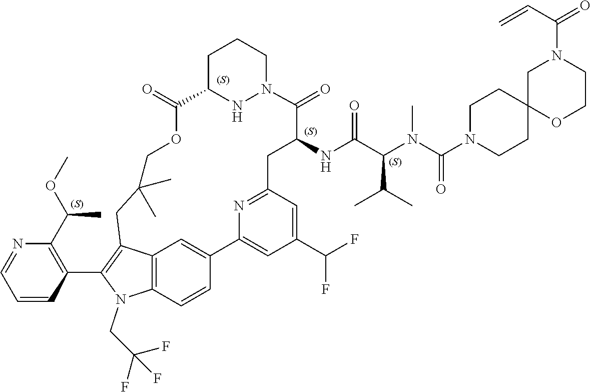 |
| BA542 | 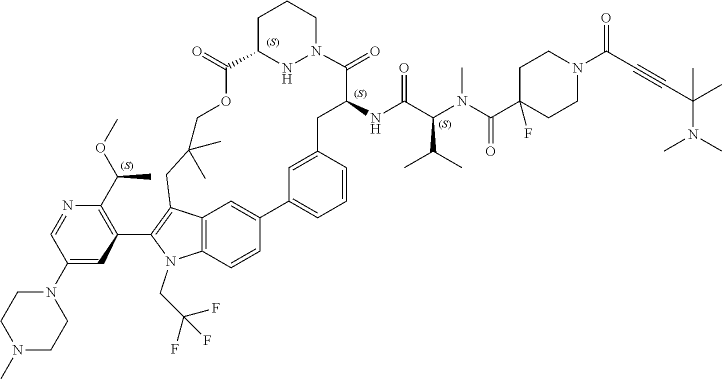 |
| BA543 | 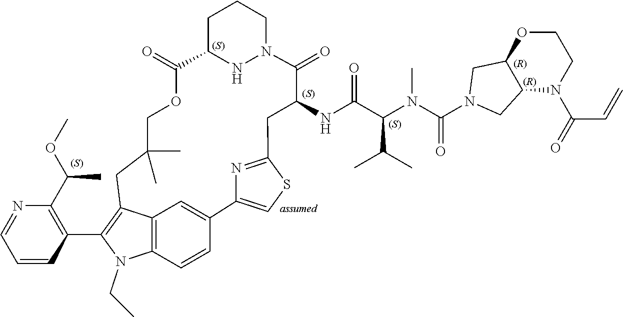 |
| BA544 | 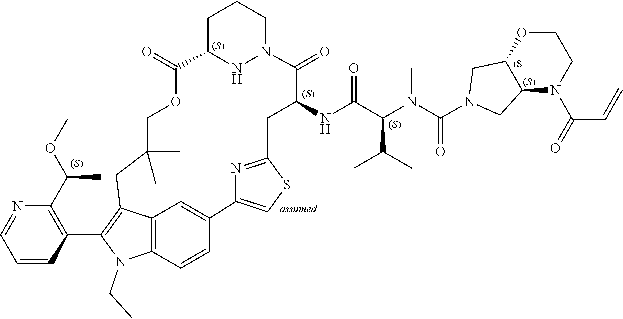 |
| BA545 | 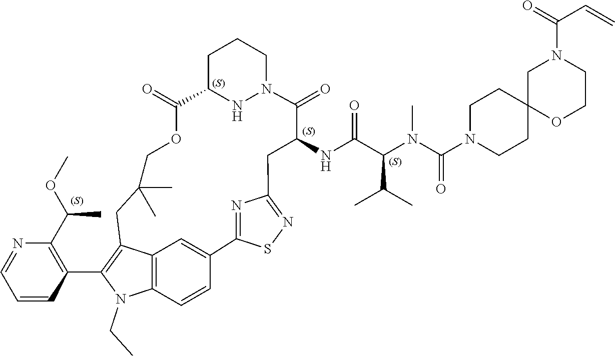 |
| BA546 | 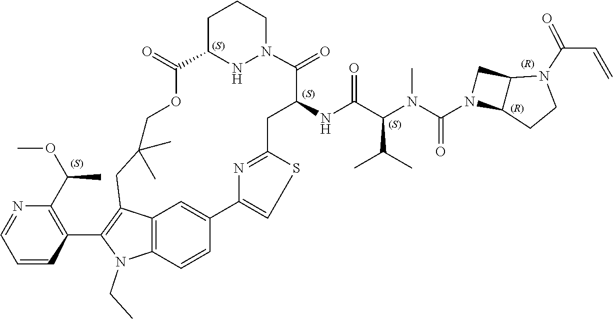 |
| BA547 | 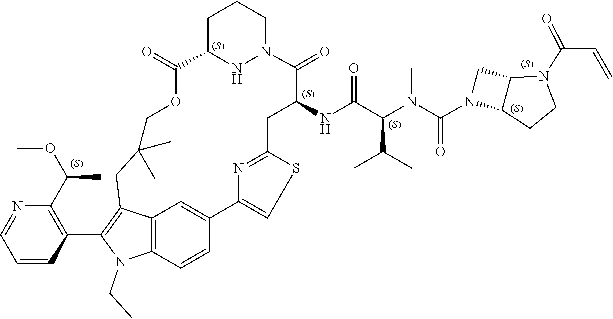 |
| BA548 | 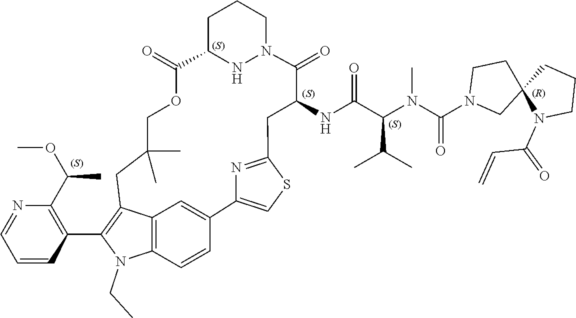 |
| BA549 | 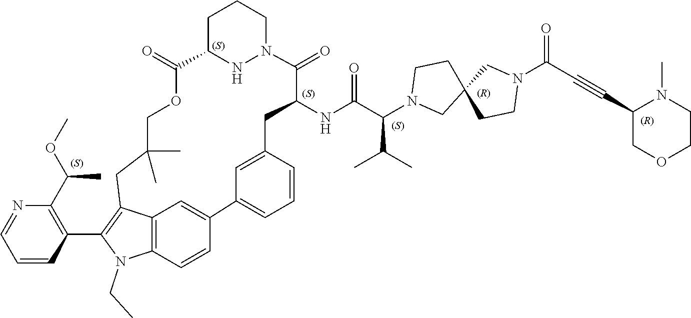 |
| BA550 | 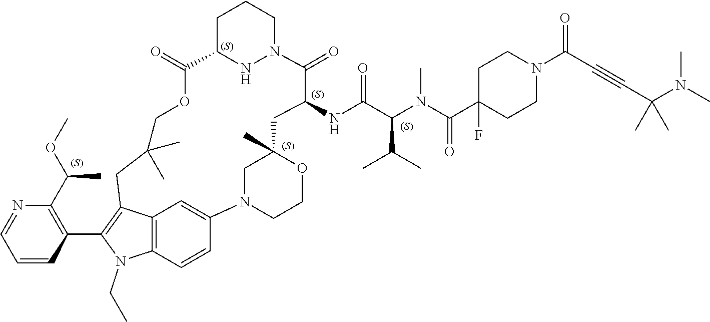 |
| BA551 | 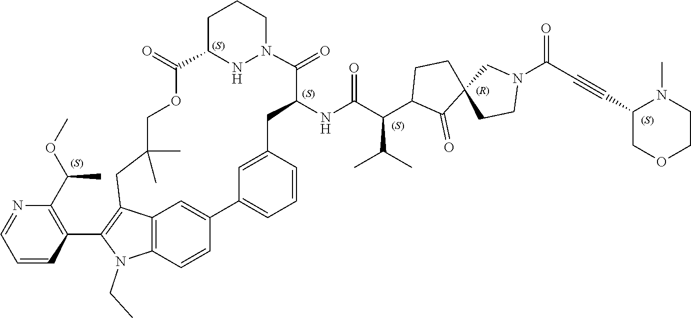 |
| BA552 | 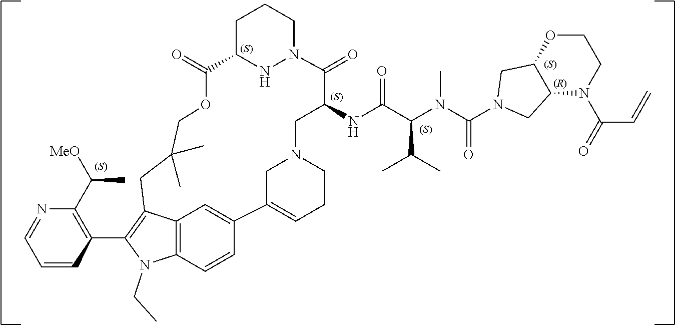 |
| BA553 | 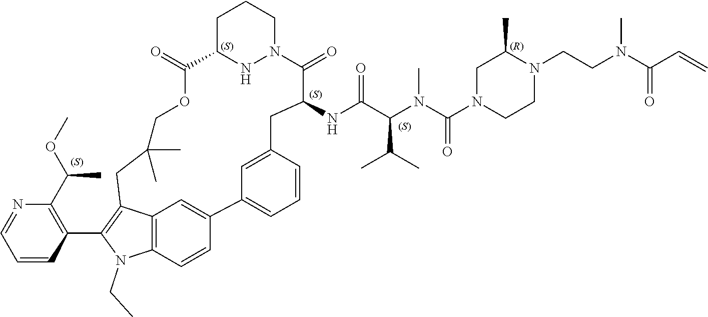 |
| BA554 | 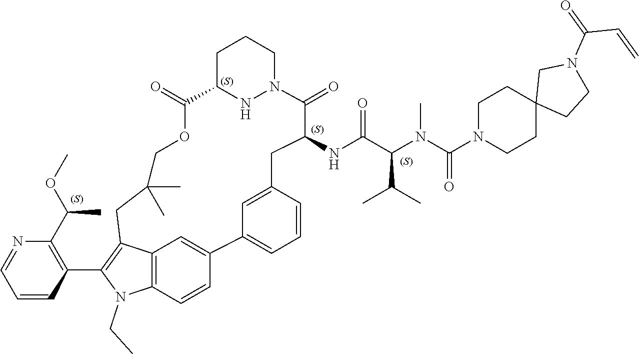 |
| BA555 | 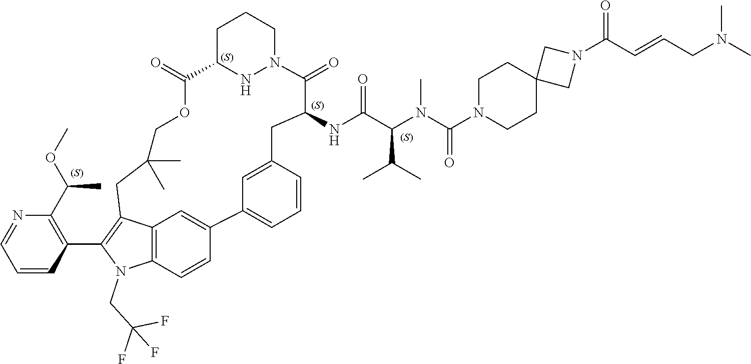 |
| BA556 | 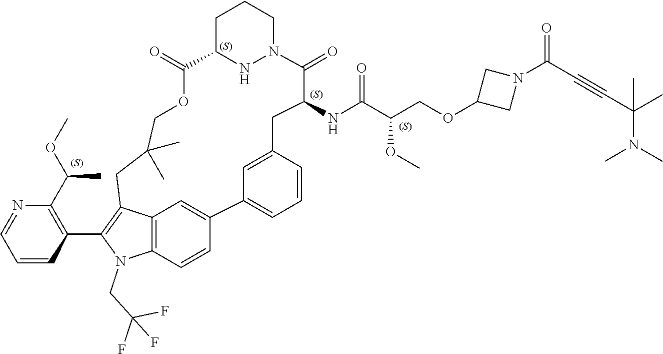 |
| BA557 | 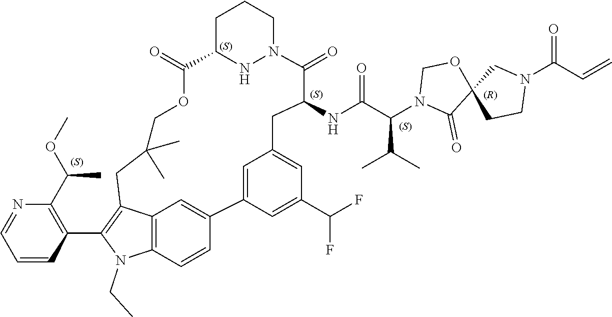 |
| BA558 | 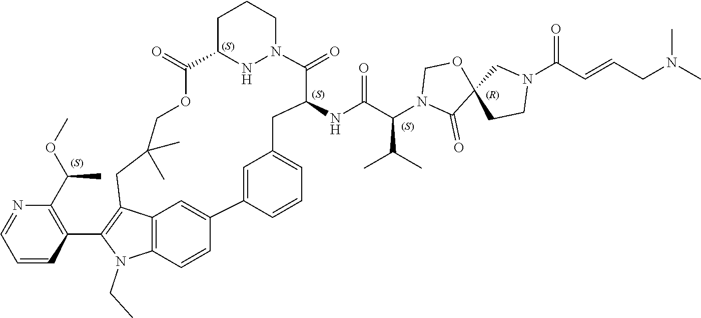 |
| BA559 | 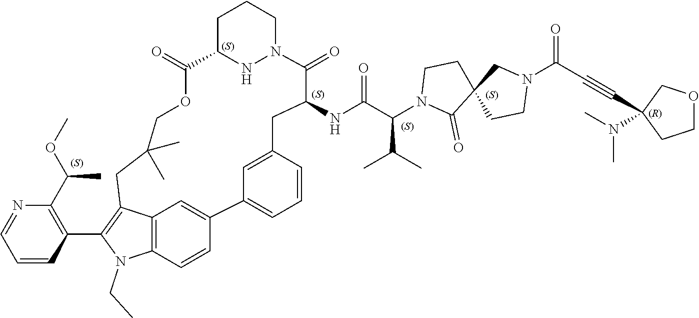 |
| BA560 | 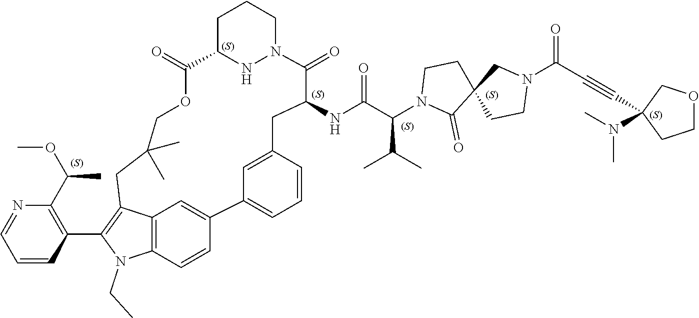 |
| BA561 | 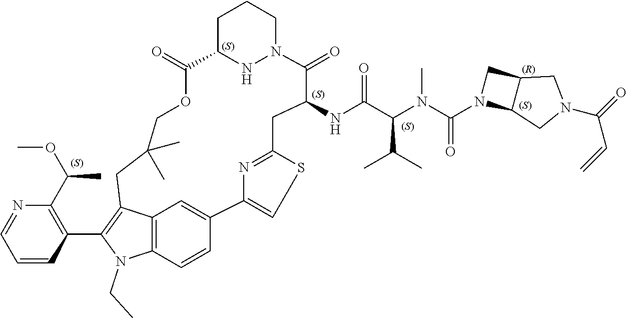 |
| BA562 | 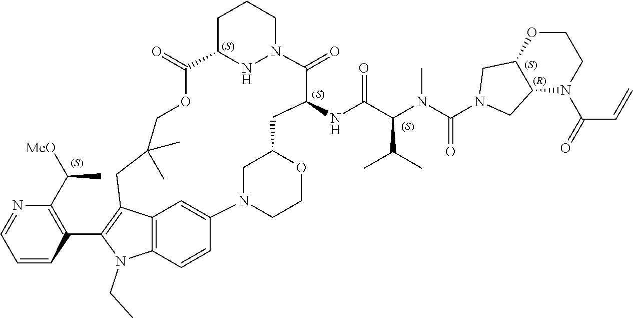 |
| BA563 | 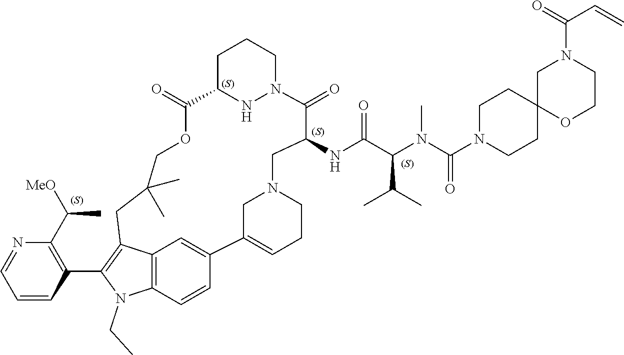 |
| BA564 | 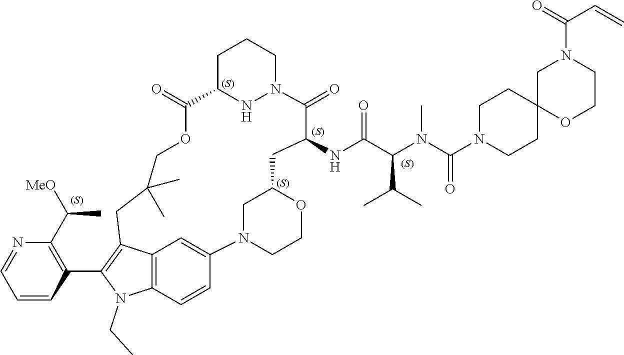 |
| BA565 | 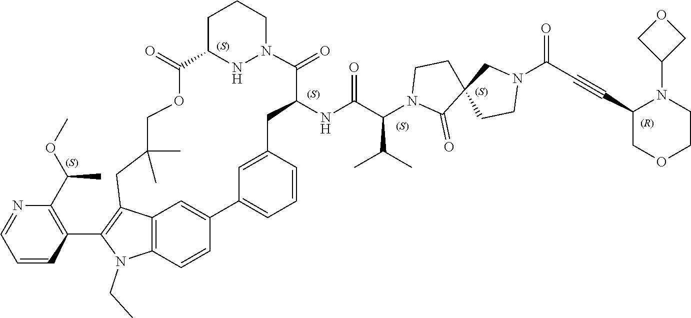 |
| BA566 | 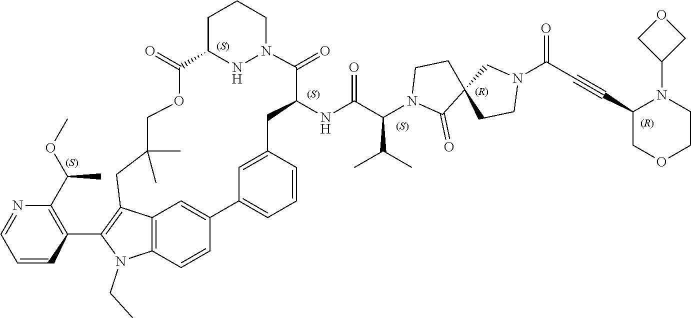 |
| BA567 | 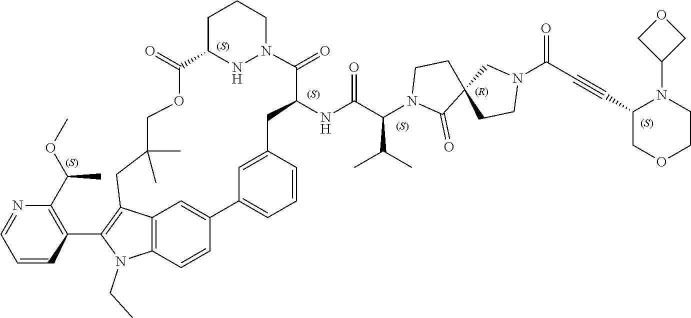 |
| BA568 | 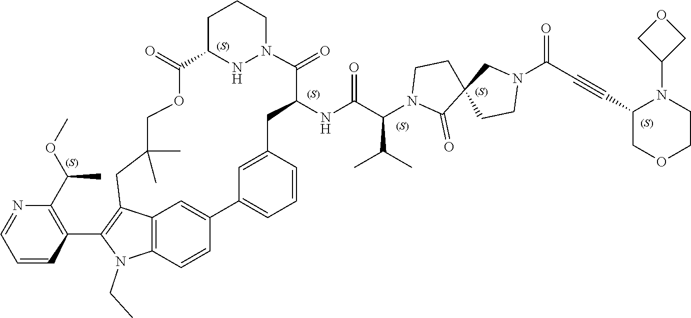 |
| BA569 | 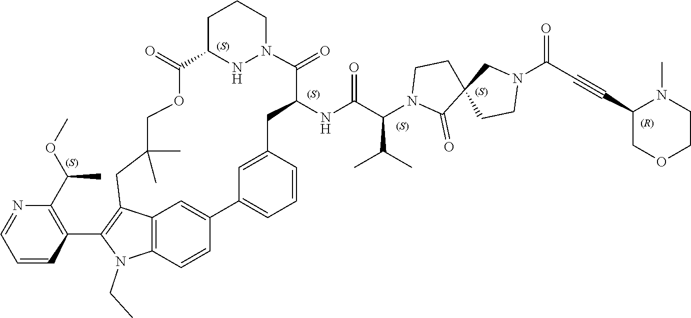 |
| BA570 | 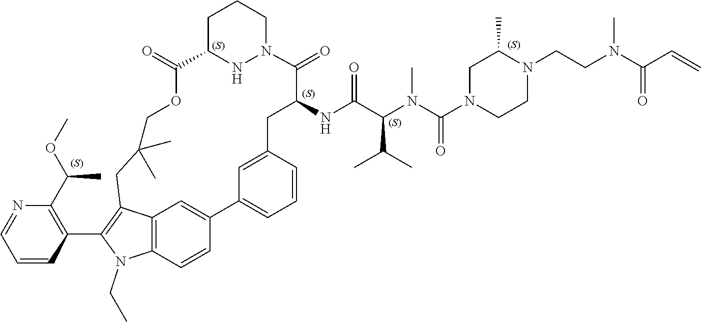 |
| BA571 | 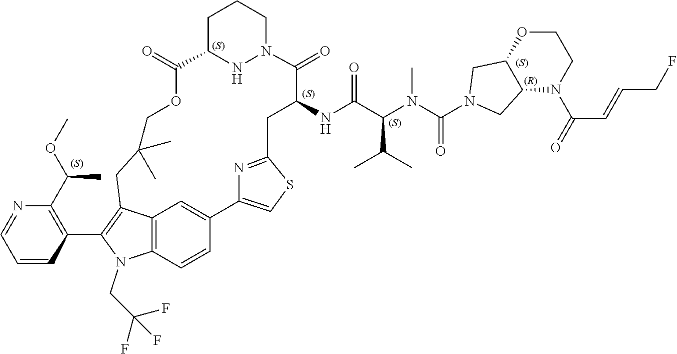 |
| BA572 | 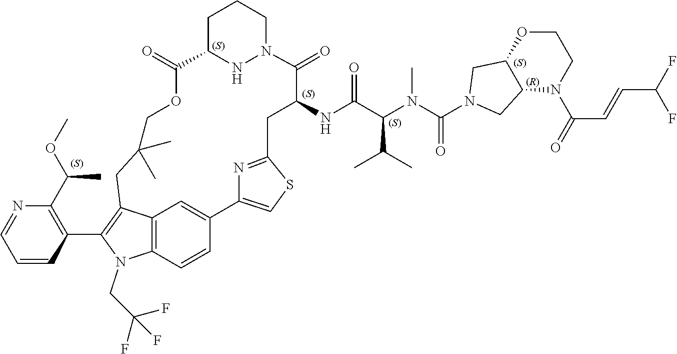 |
| BA573 | 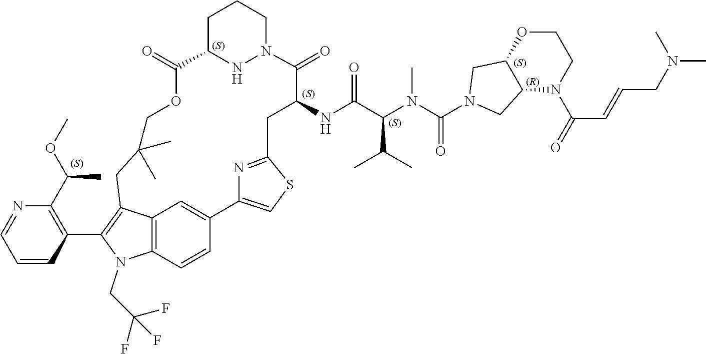 |
| BA574 | 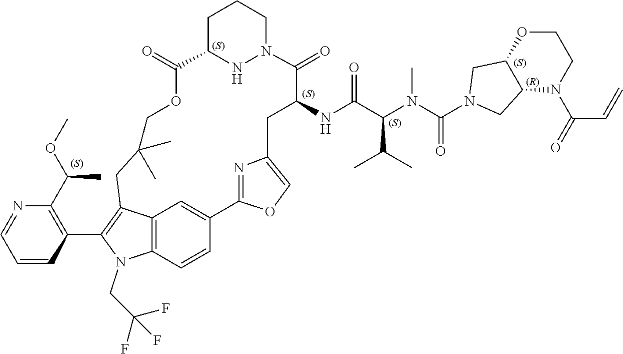 |
| BA575 | 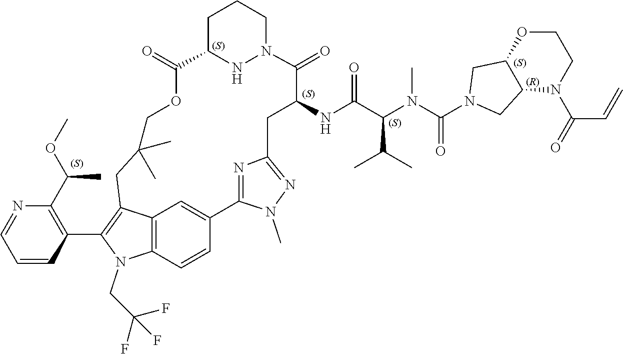 |
| BA576 | 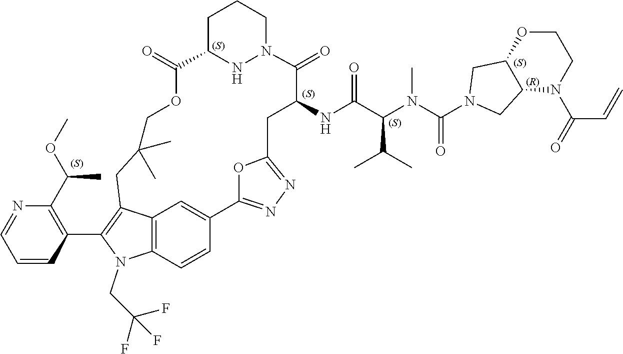 |
| BA577 | 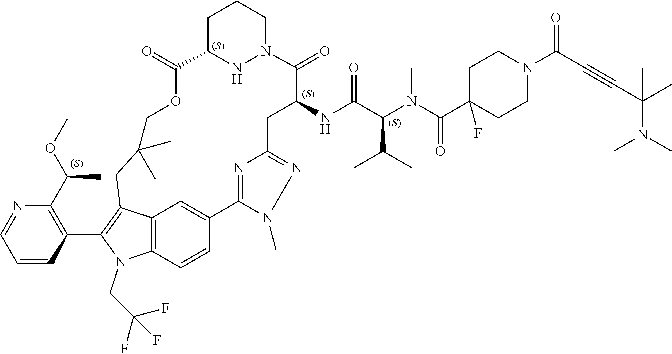 |
| BA578 | 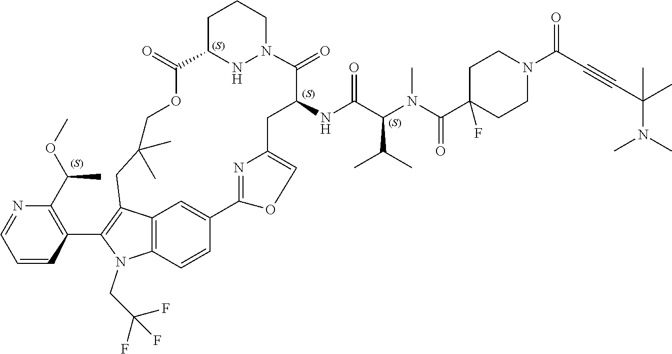 |
| BA579 | 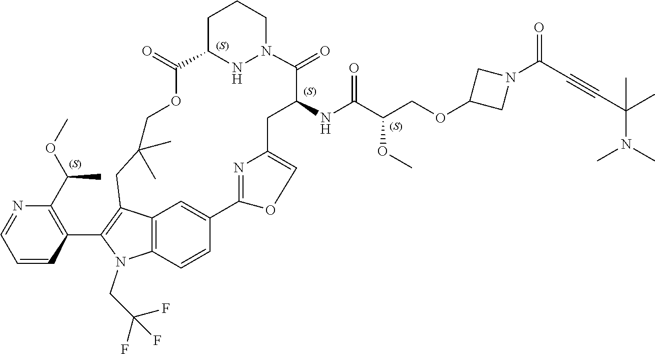 |
| BA580 | 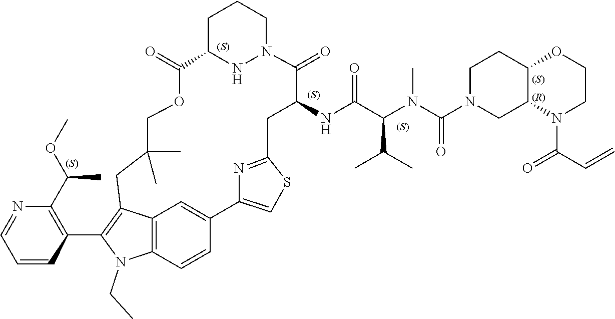 |
| BA581 | 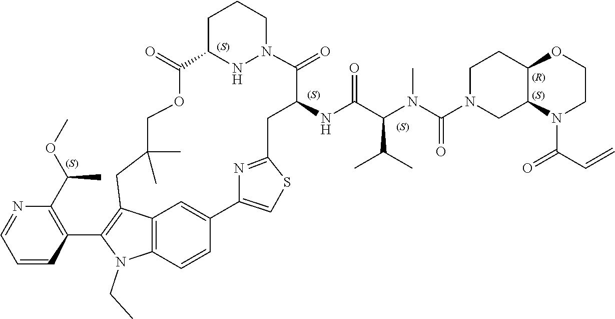 |
| BA582 | 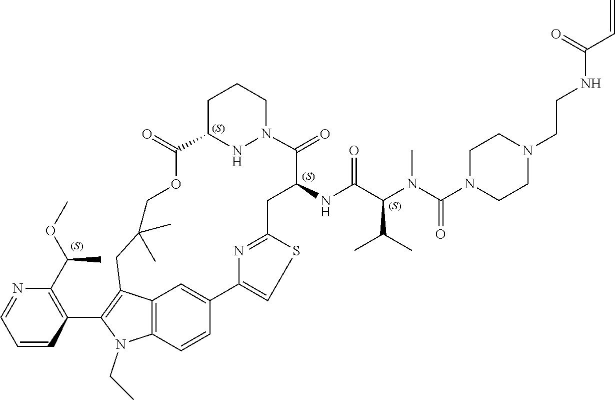 |
| BA583 | 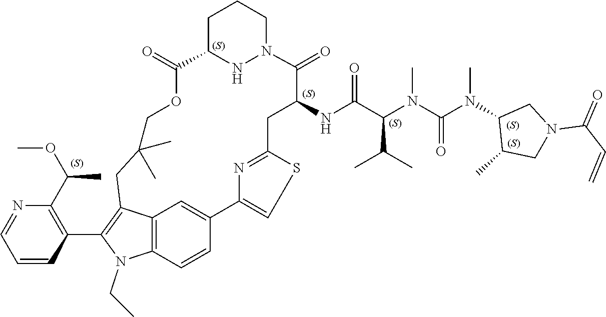 |
| BA584 | 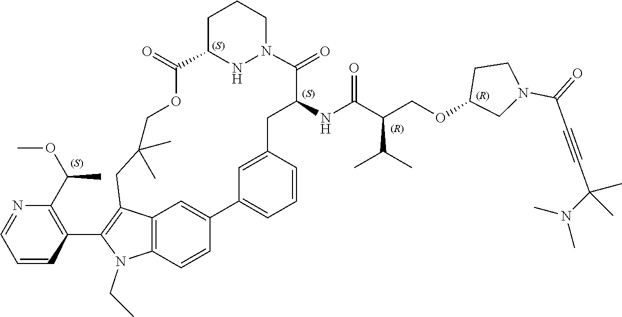 |
| BA585 | 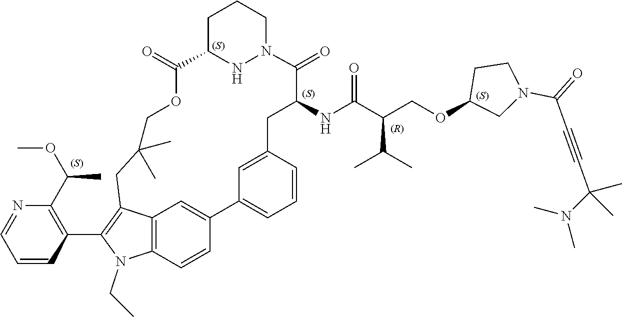 |
| BA586 | 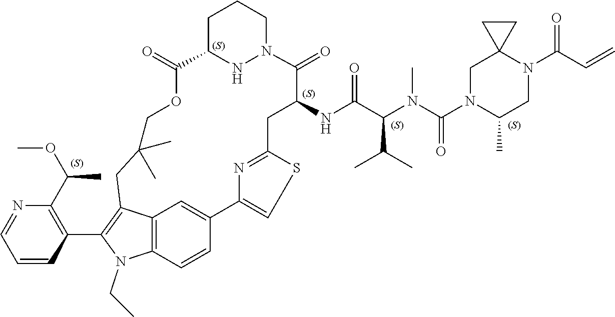 |
| BA587 | 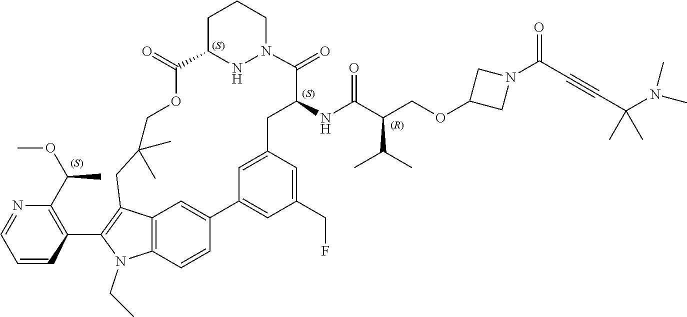 |
| BA588 | 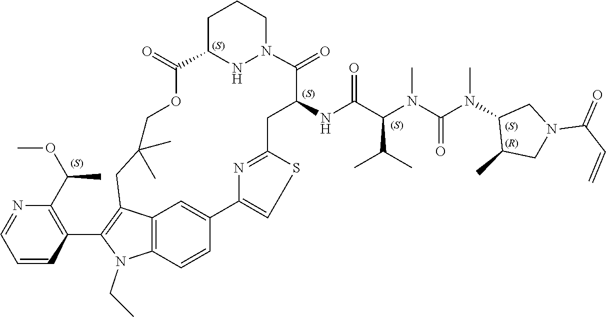 |
| BA589 | 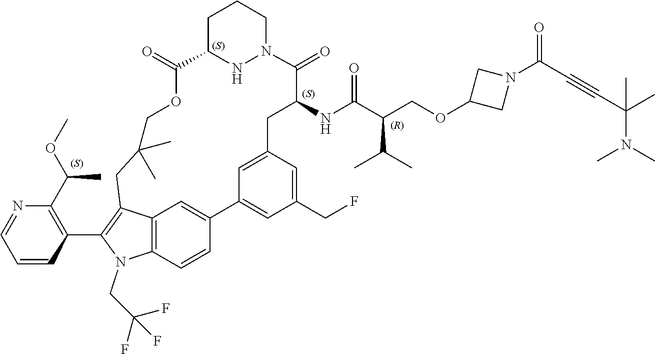 |
| BA590 | 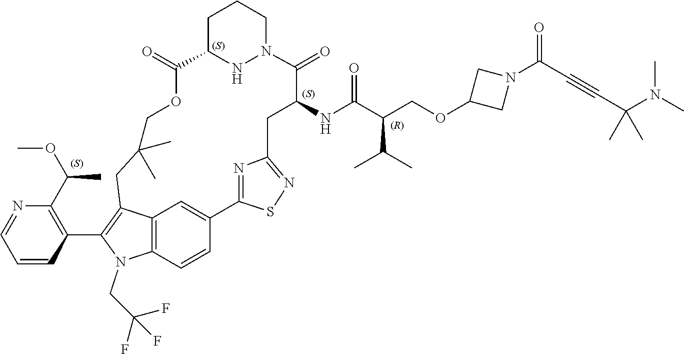 |
| BA591 | 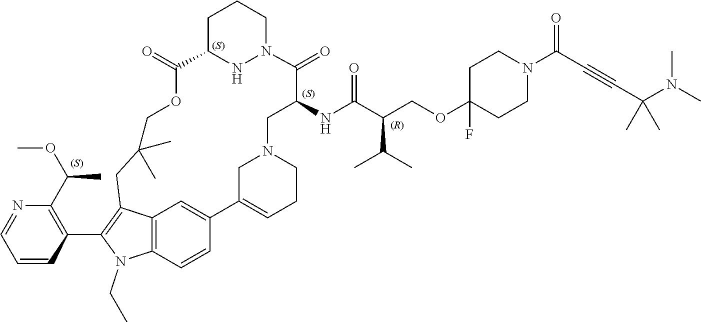 |
| BA592 | 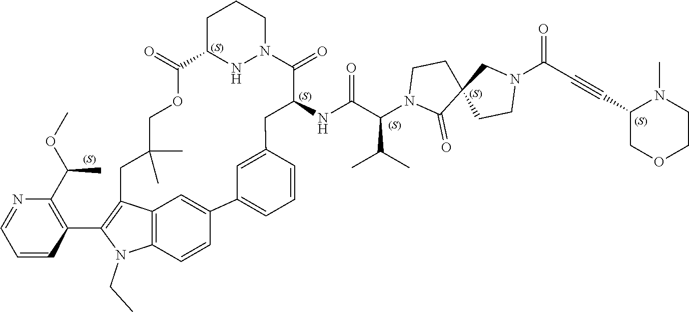 |
| BA593 | 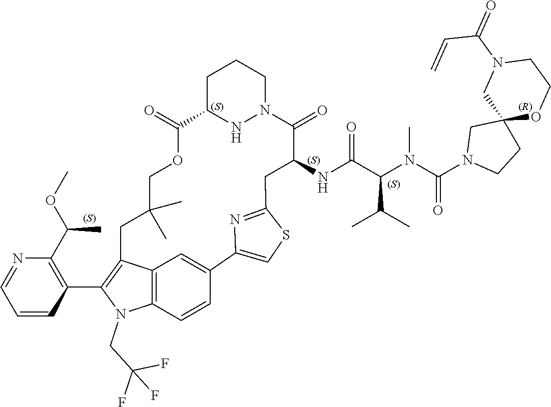 |
| BA594 | 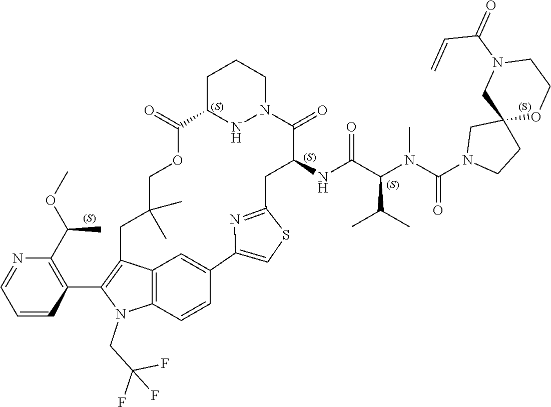 |
| BA595 | 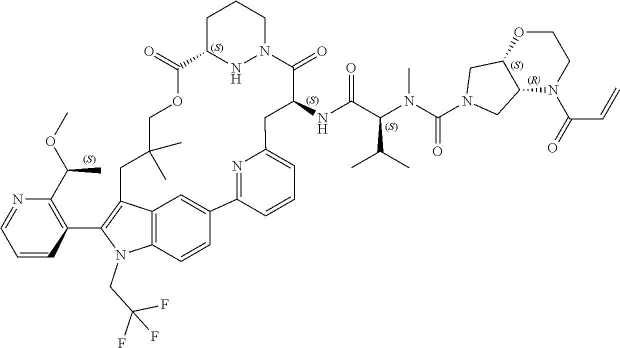 |
| BA596 | 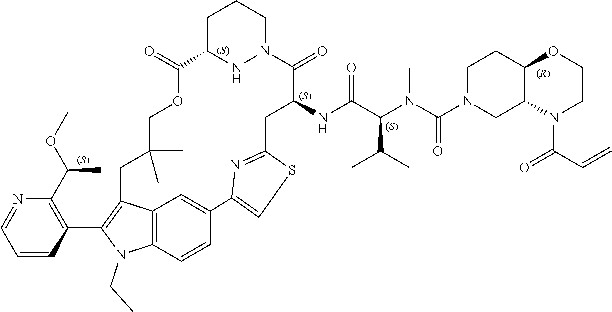 |
| BA597 | 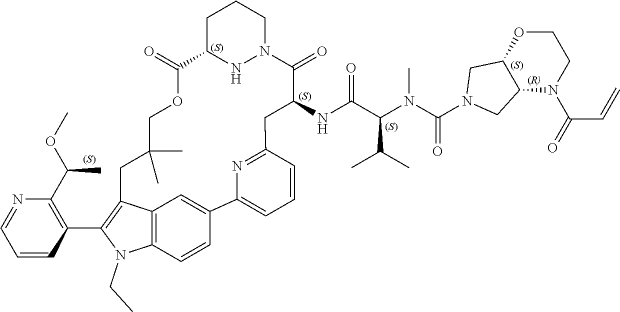 |
| BA598 | 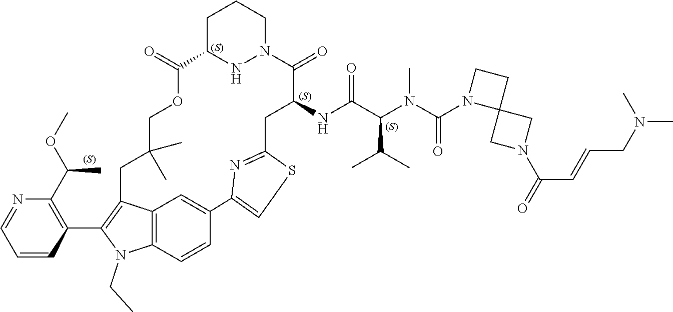 |
| BA599 | 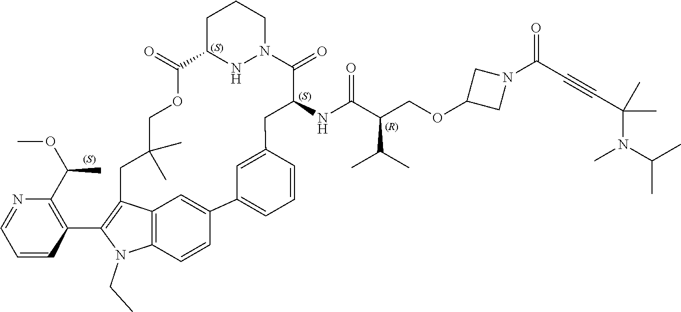 |
| BA600 | 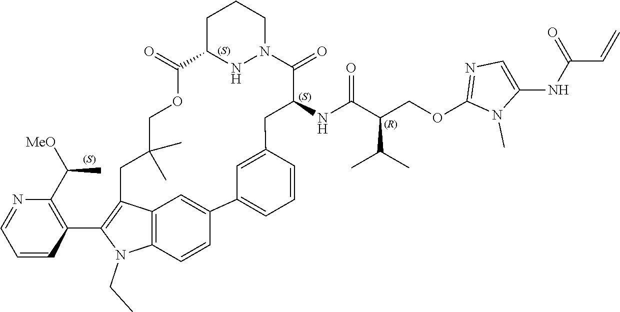 |
| BA601 | 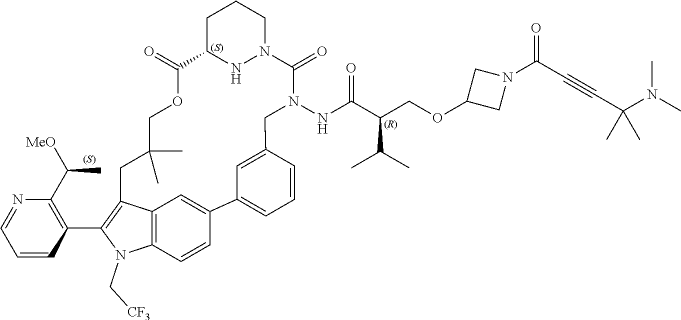 |
| BA602 | 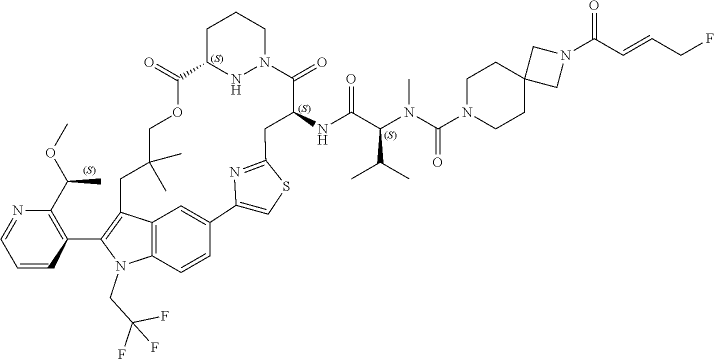 |
| BA603 | 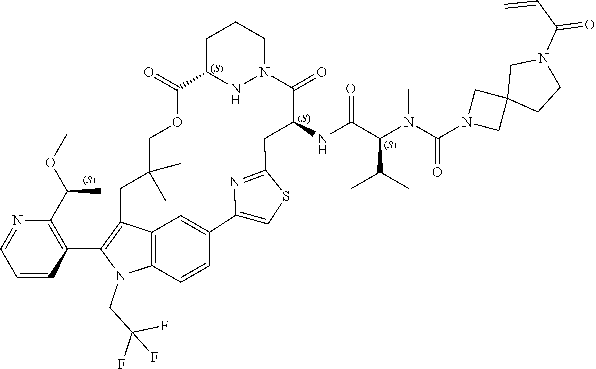 |
| BA604 | 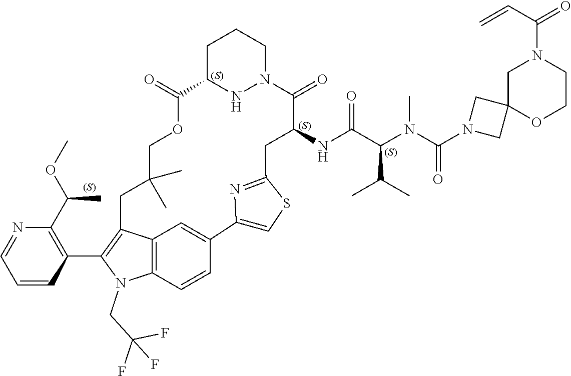 |
| BA605 | 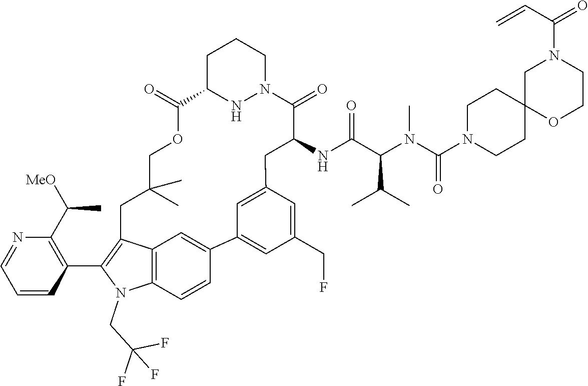 |
| BA606 | 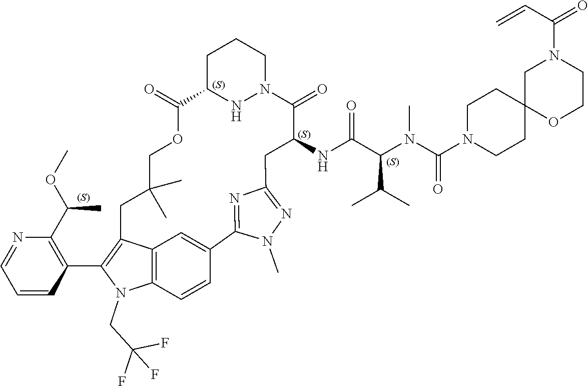 |
| BA607 | 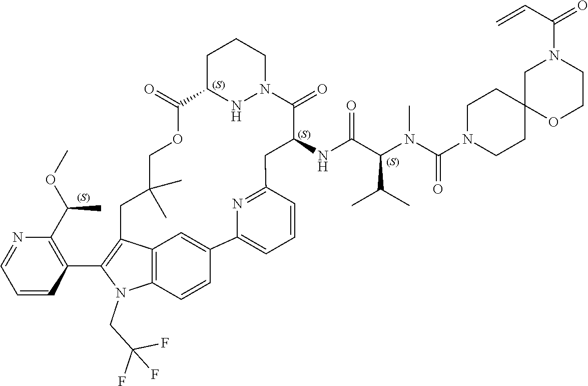 |
| BA608 | 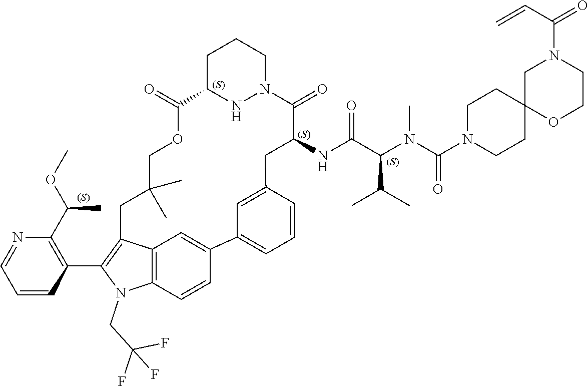 |
| BA609 | 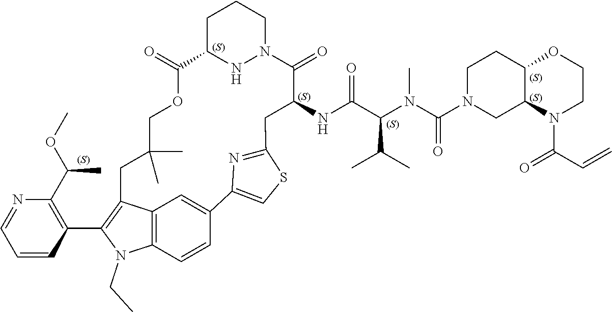 |
| BA610 | 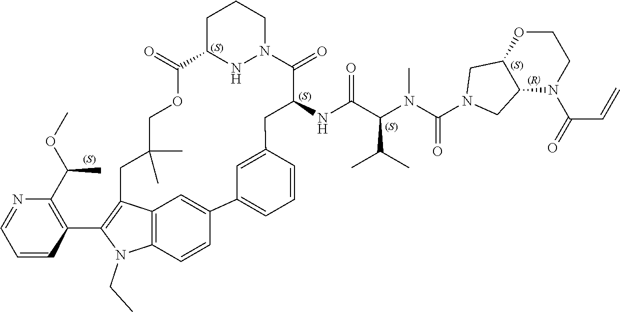 |
| BA611 | 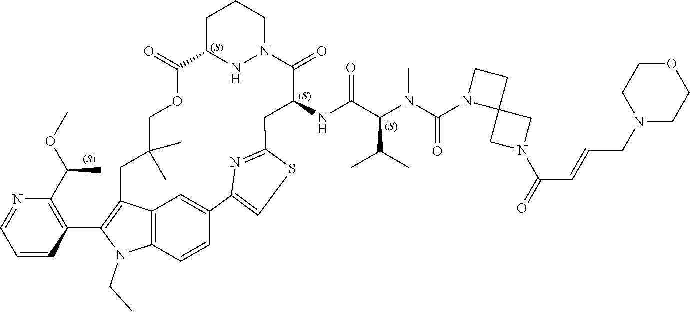 |
| BA612 | 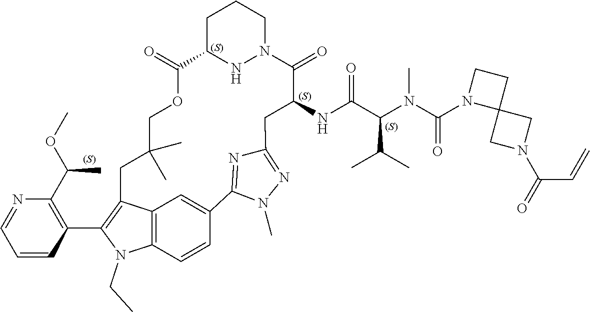 |
| BA613 | 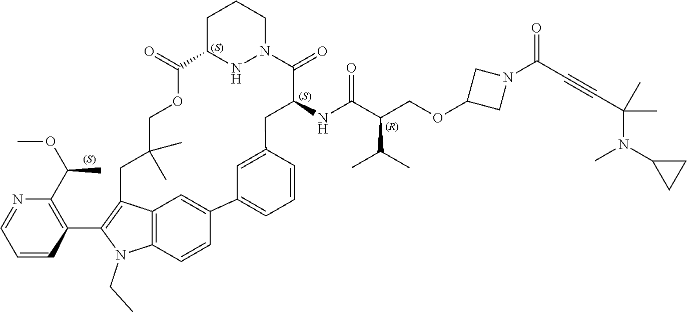 |
| BA614 | 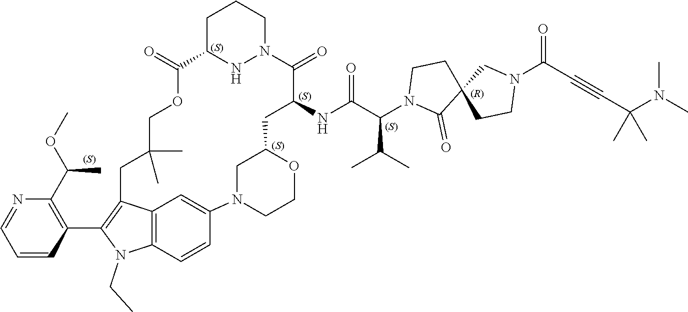 |
| BA615 | 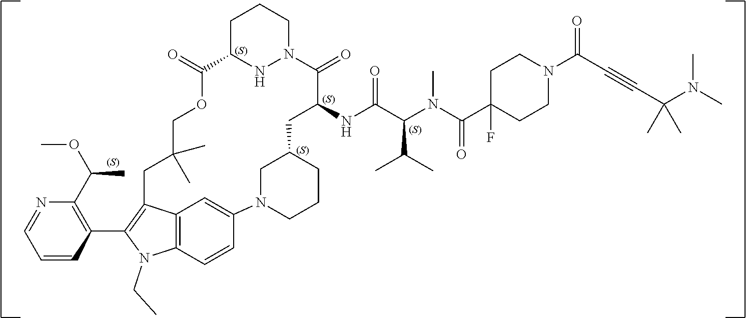 |
| BA616 | 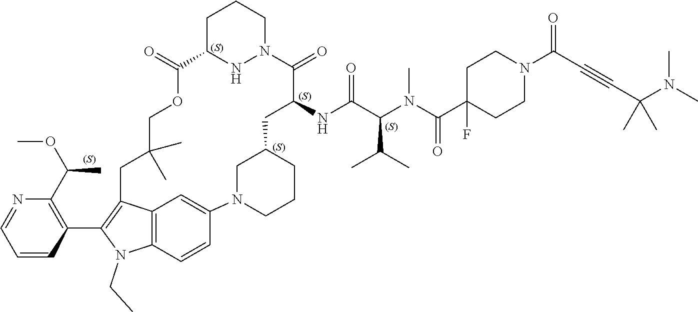 |
| BA617 | 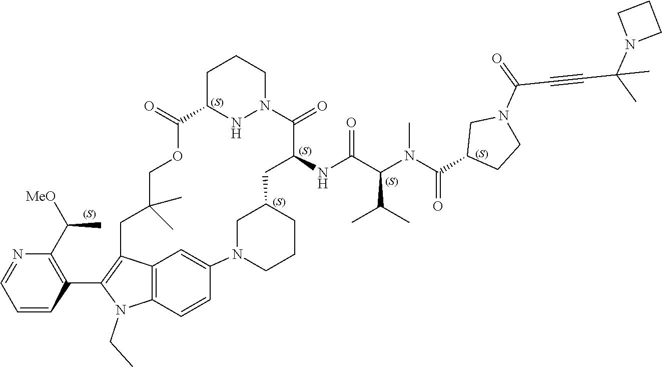 |
| BA618 | 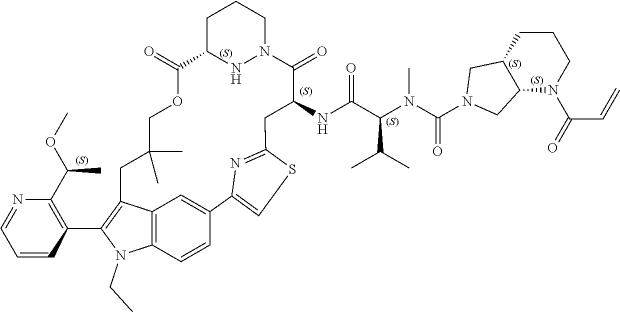 |
| BA619 | 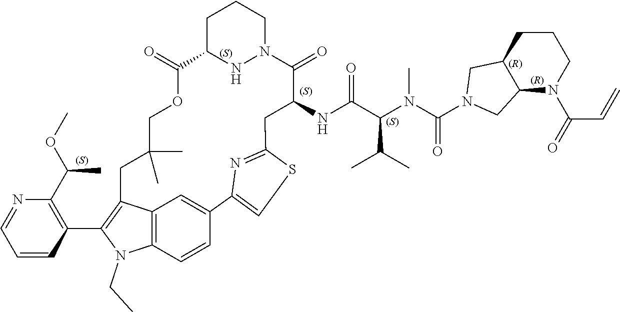 |
| BA620 | 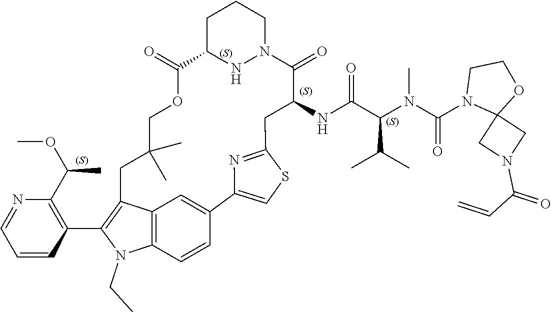 |
| BA621 | 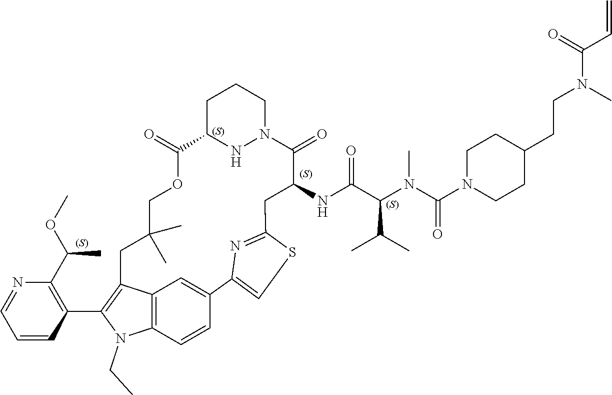 |
| BA622 | 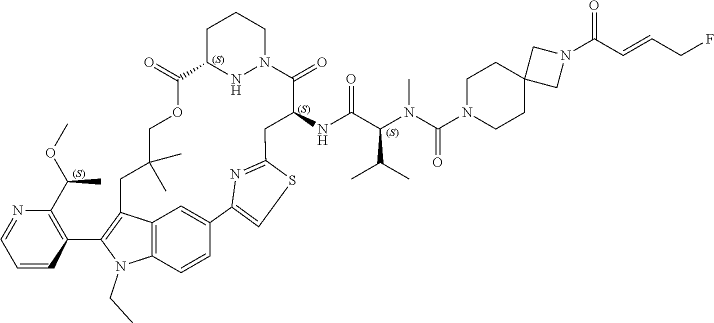 |
| BA623 | 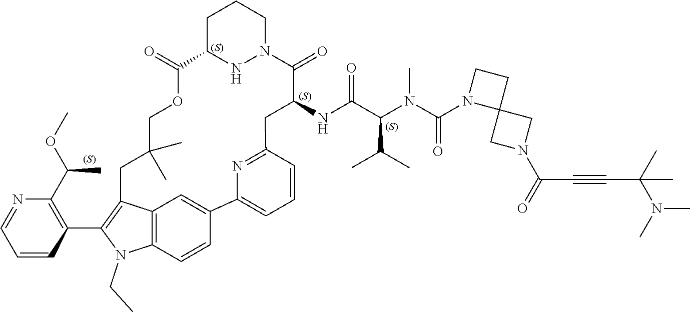 |
| BA624 | 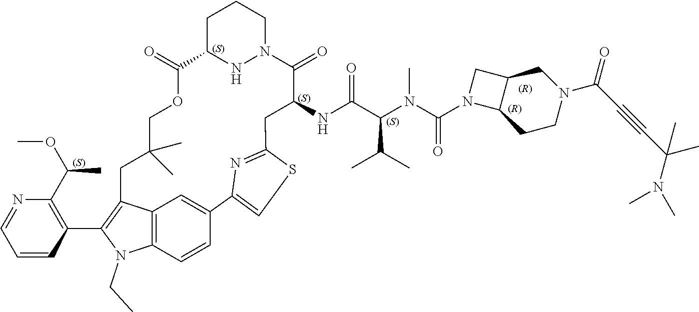 |
| BA625 | 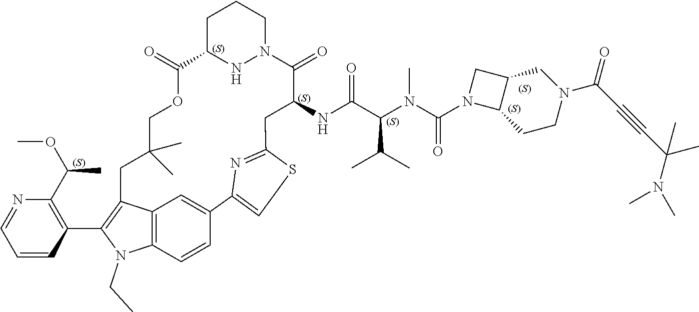 |
| BA626 | 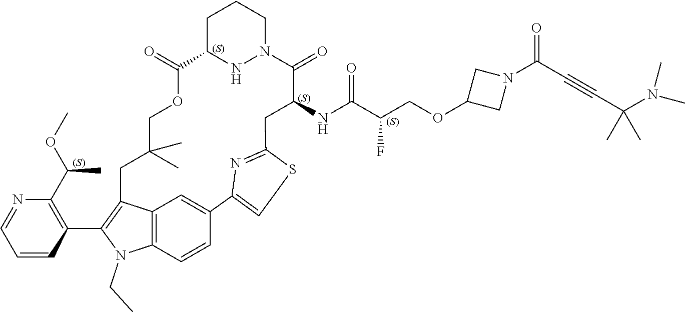 |
| BA627 | 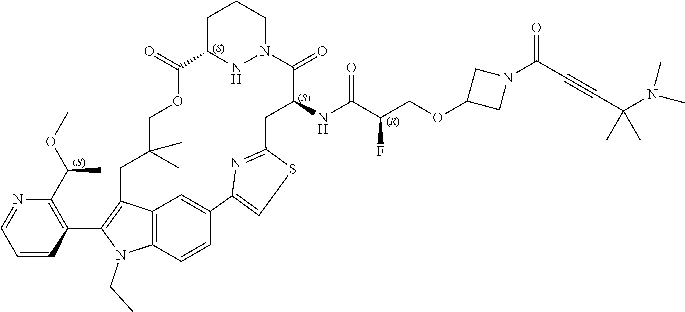 |
| BA628 | 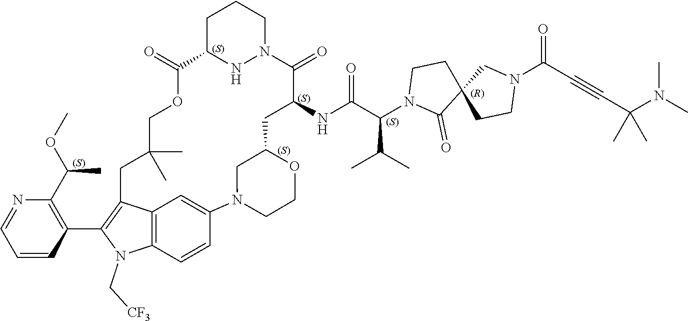 |
| BA629 | 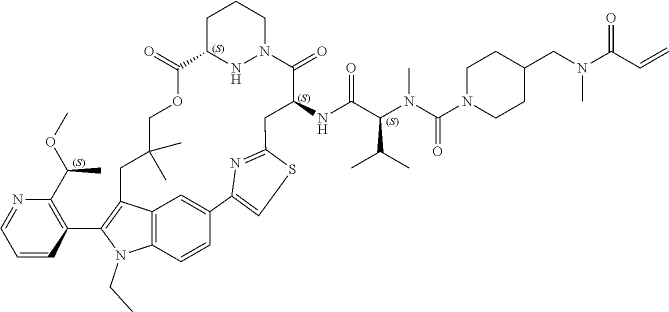 |
| BA630 | 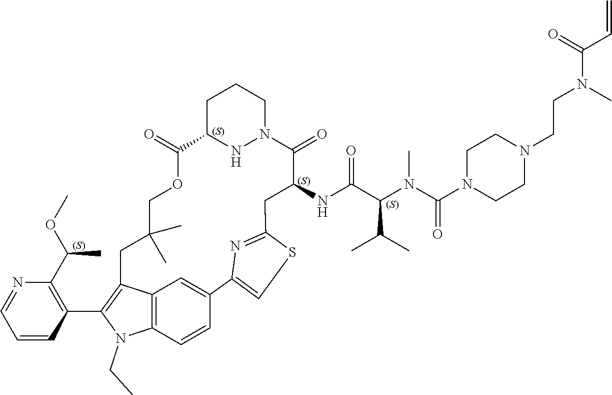 |
| BA631 | 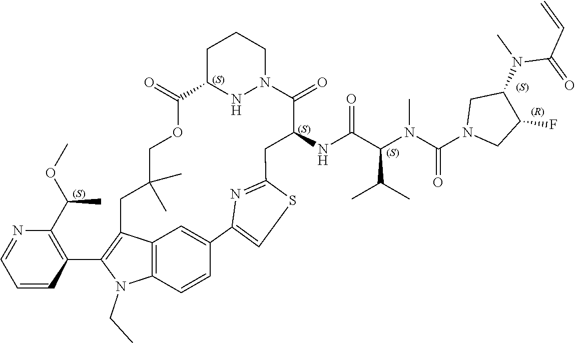 |
| BA632 | 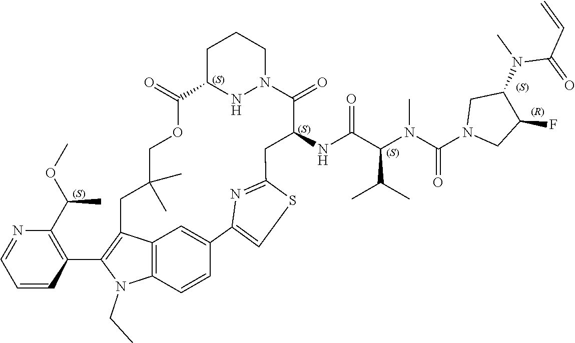 |
| BA633 | 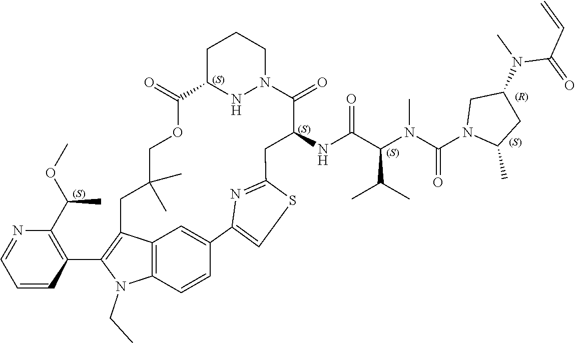 |
| BA634 | 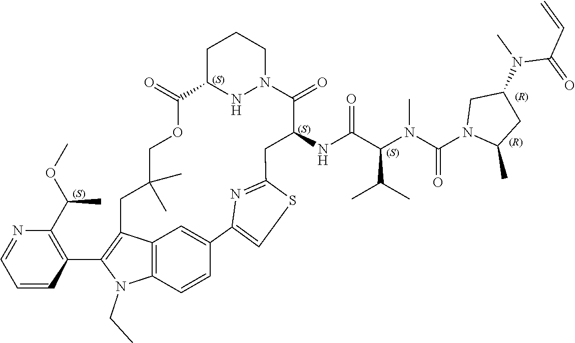 |
| BA635 | 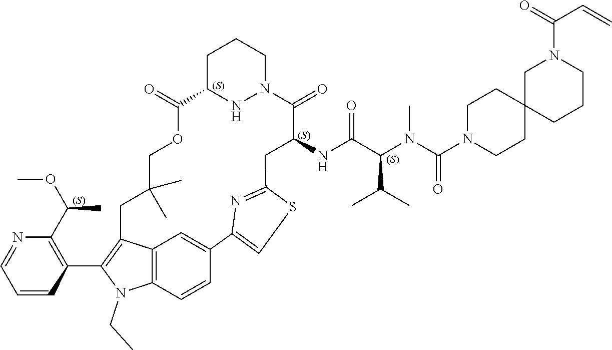 |
| BA636 | 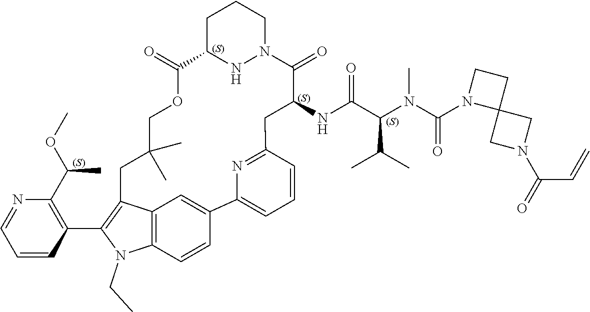 |
| BA637 | 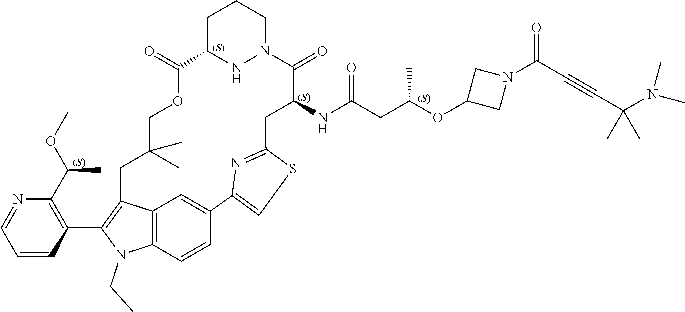 |
| BA638 | 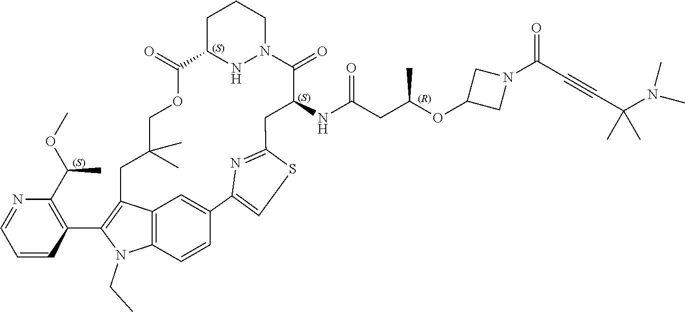 |
| BA639 | 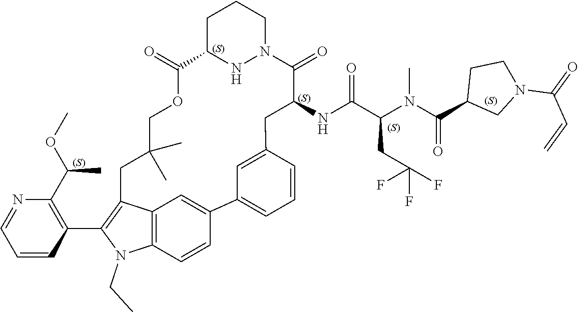 |
| BA640 | 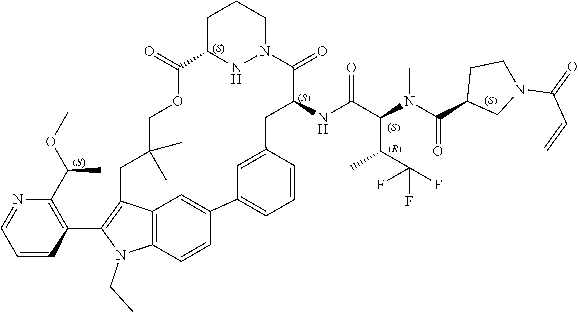 |
| BA641 | 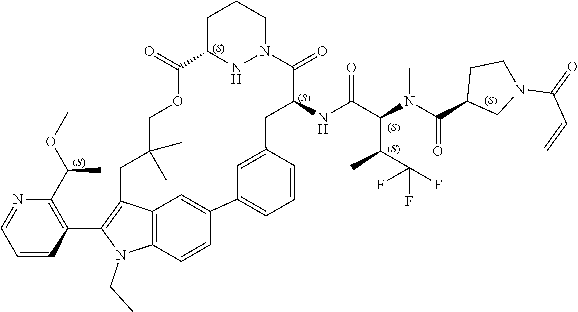 |
| BA642 | 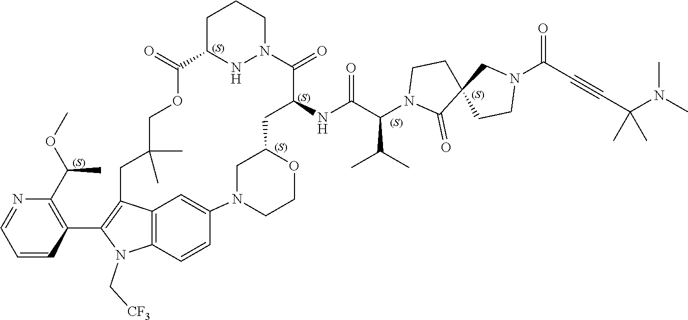 |
| BA643 | 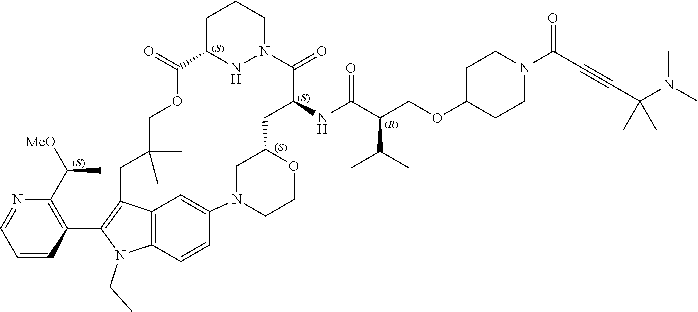 |
| BA644 | 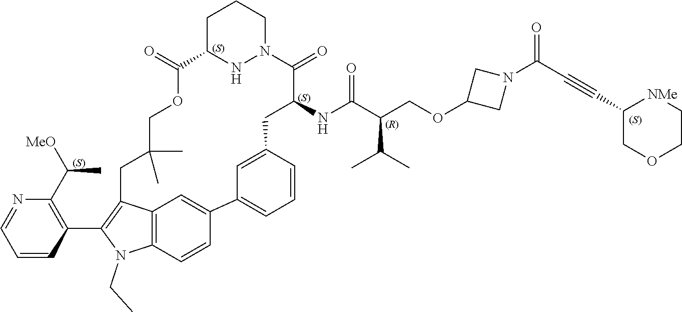 |
| BA645 | 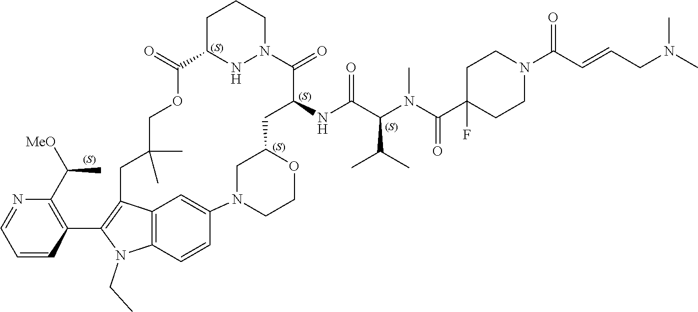 |
| BA646 | 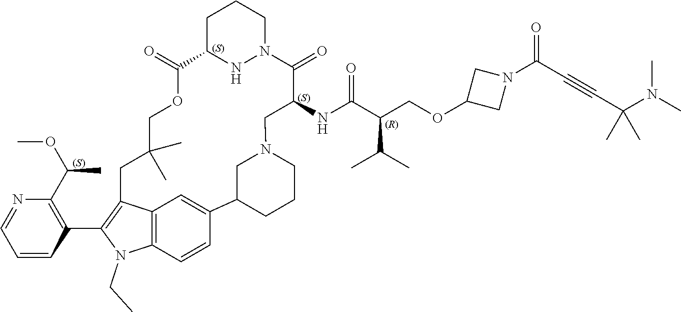 |
| BA647 | 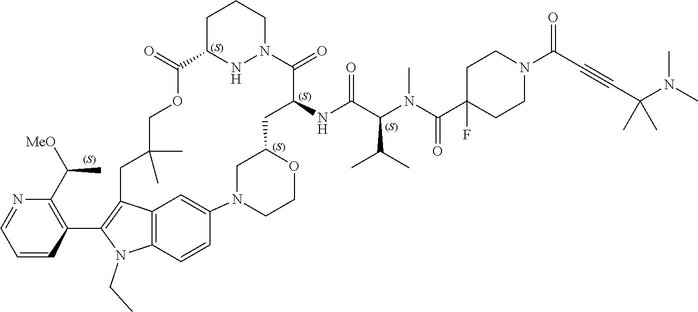 |
| BA648 | 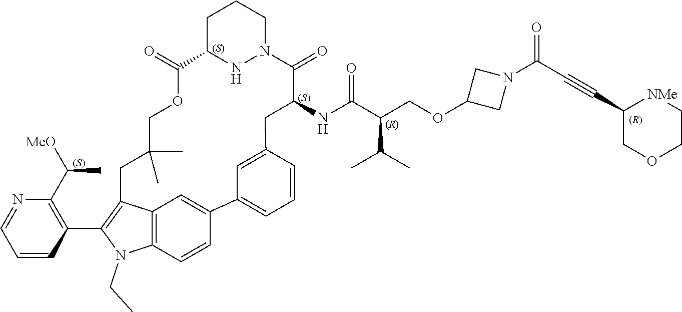 |
| BA649 | 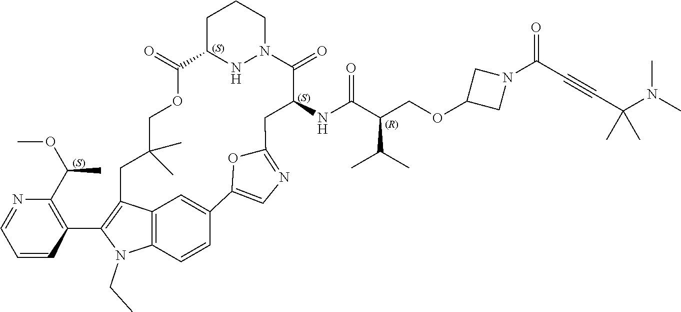 |
| BA650 | 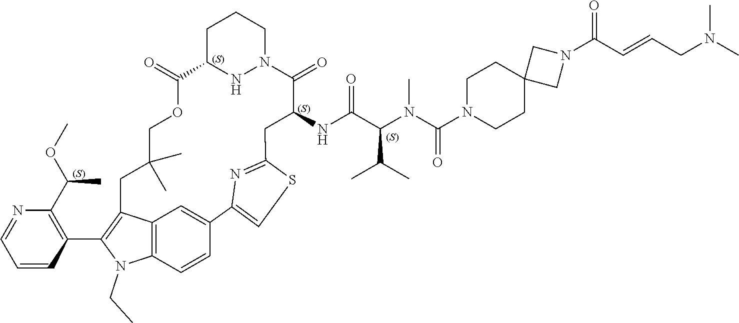 |
| BA651 | 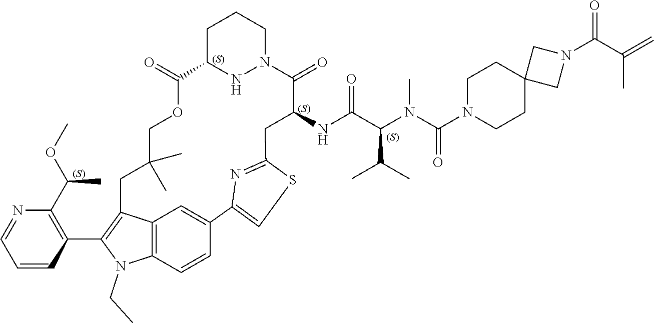 |
| BA652 | 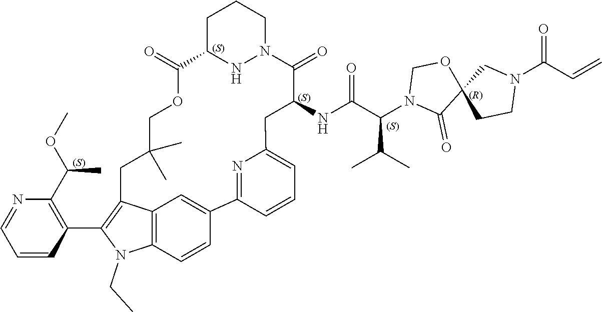 |
| BA653 | 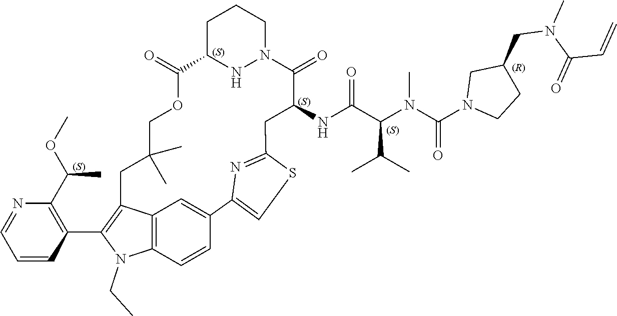 |
| BA654 | 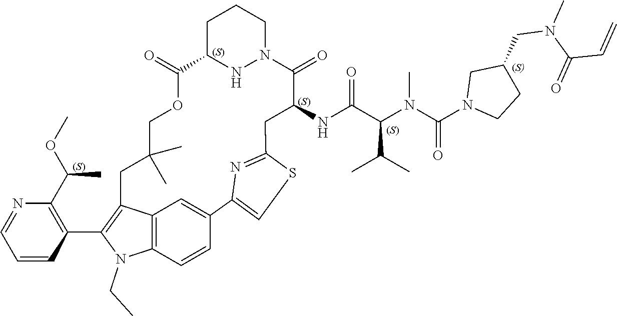 |
| BA655 | 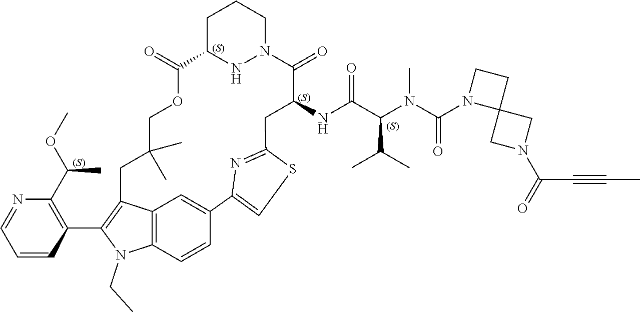 |
| BA656 | 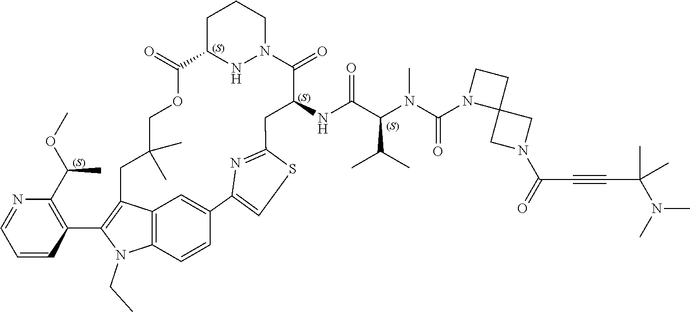 |
| BA657 | 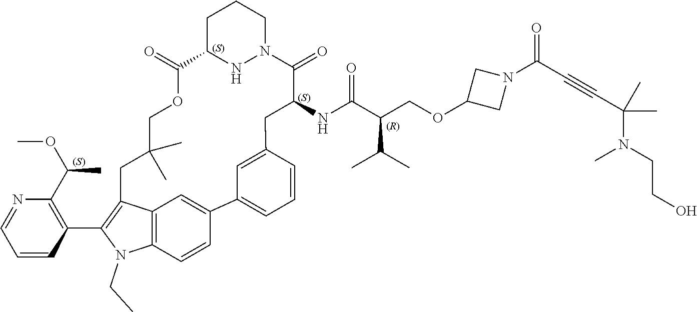 |
| BA658 | 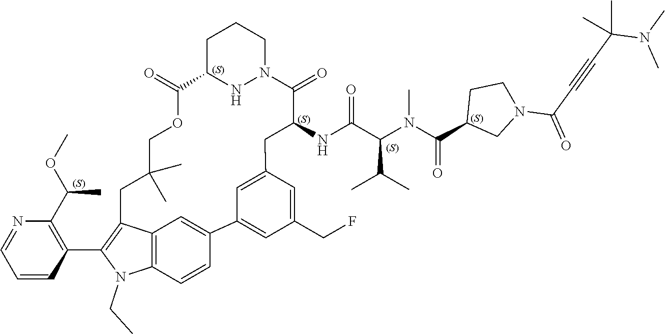 |
| BA659 | 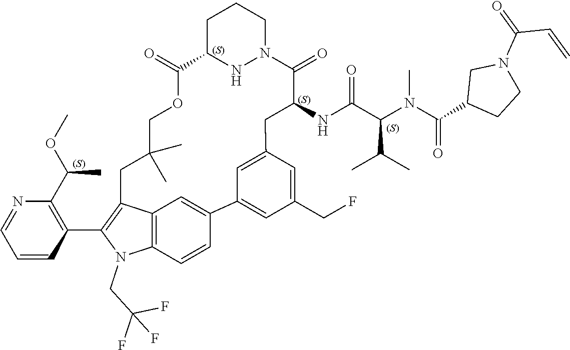 |
| BA660 | 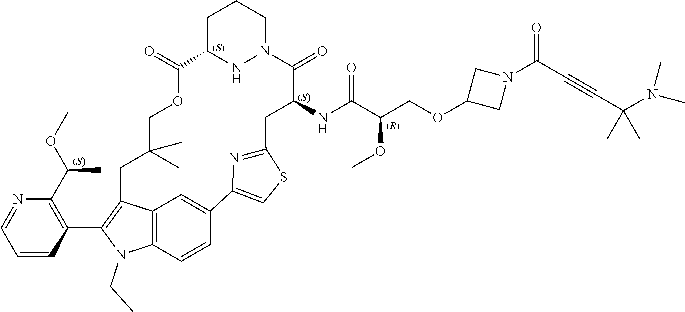 |
| BA661 | 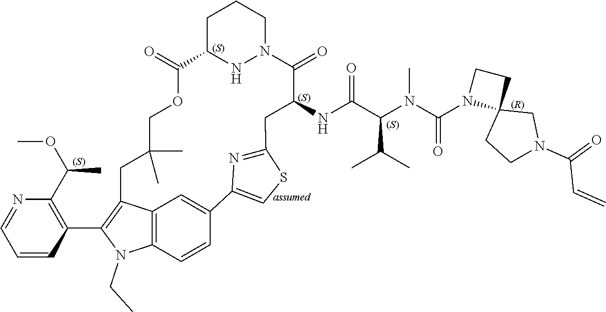 |
| BA662 | 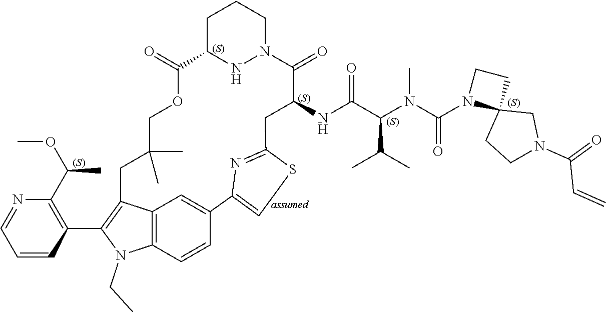 |
| BA663 | 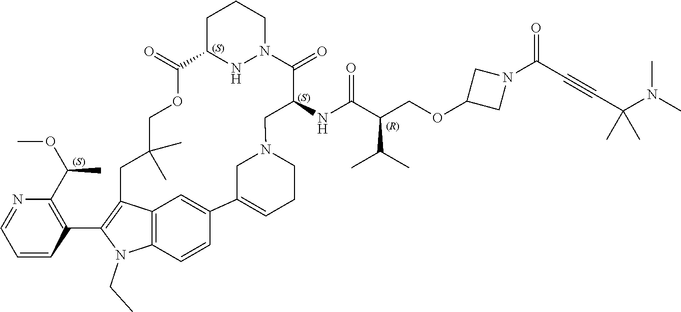 |
| BA664 | 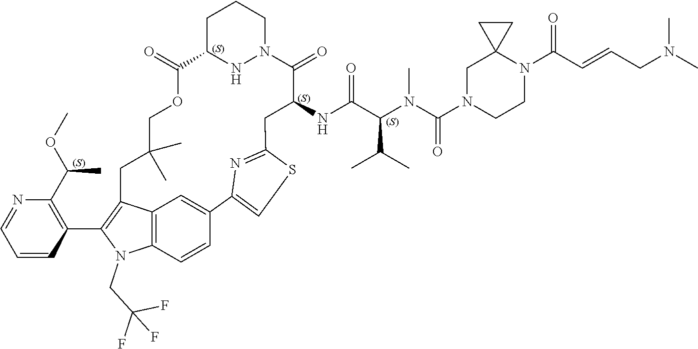 |
| BA665 | 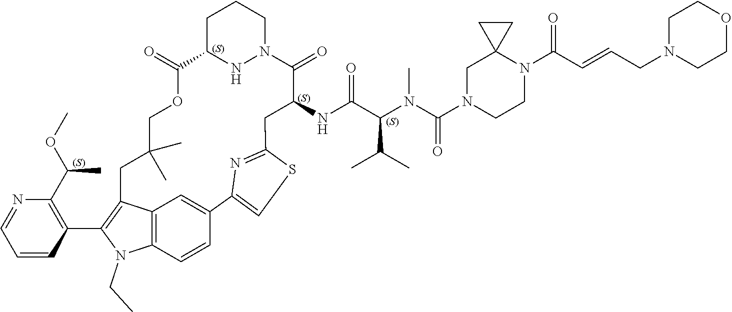 |
| BA666 | 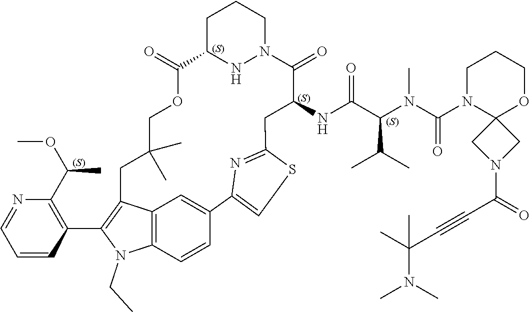 |
| BA667 | 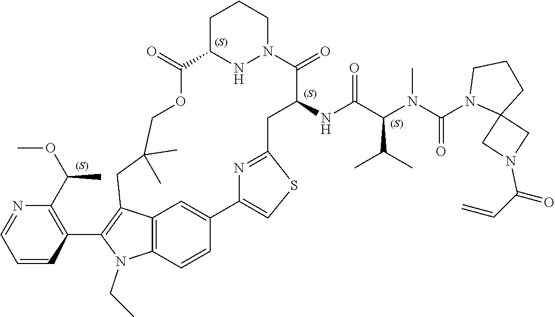 |
| BA668 | 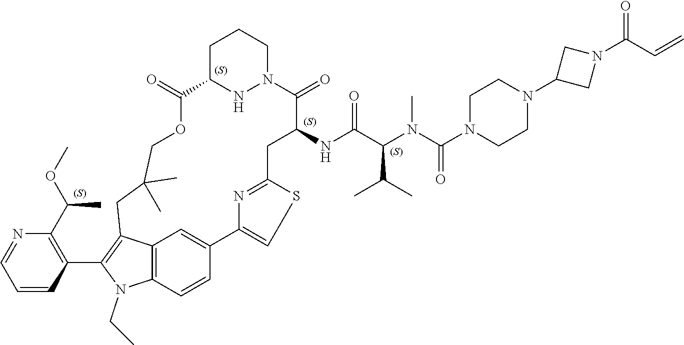 |
| BA669 | 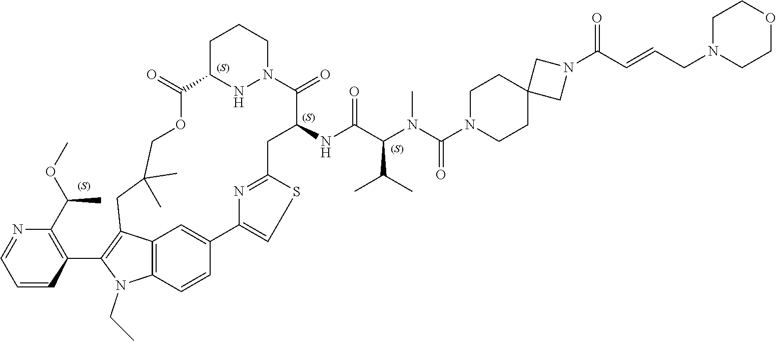 |
| BA670 | 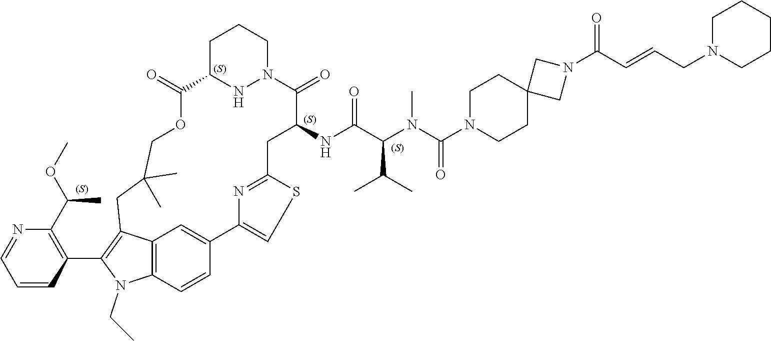 |
| BA671 | 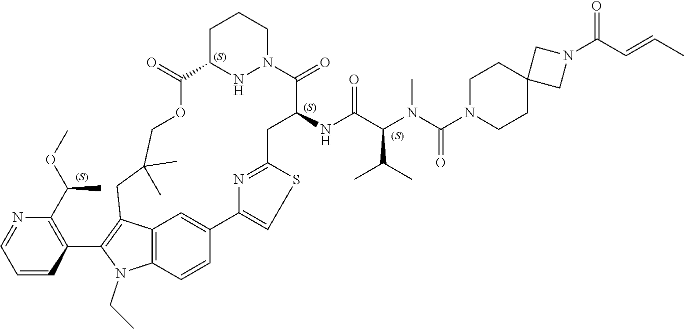 |
| BA672 | 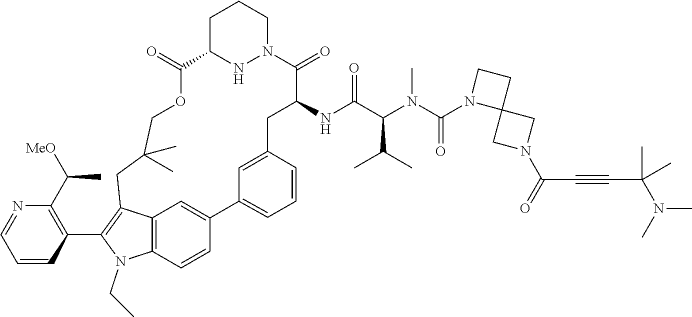 |
| BA673 | 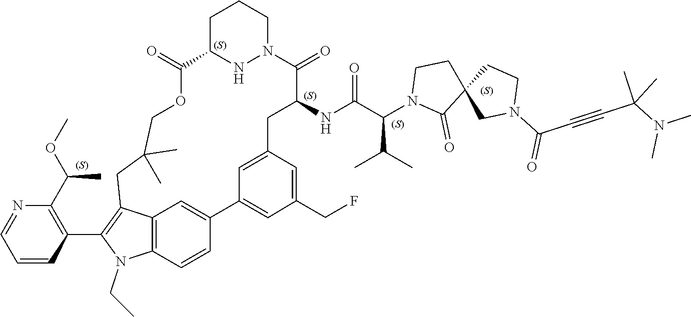 |
| BA674 | 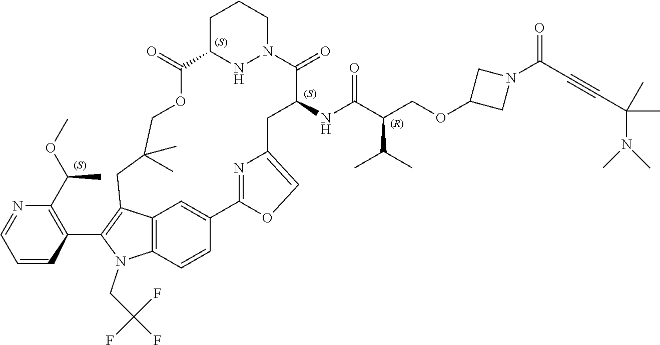 |
| BA675 | 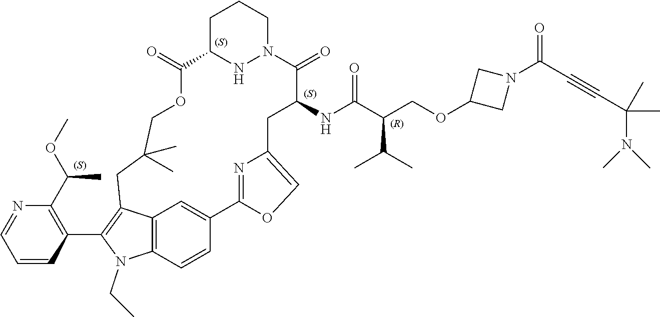 |
| BA676 | 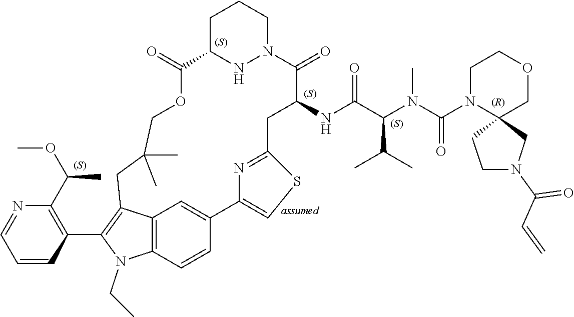 |
| BA677 | 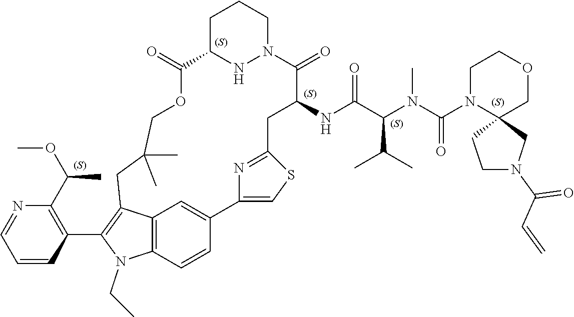 |
| BA678 | 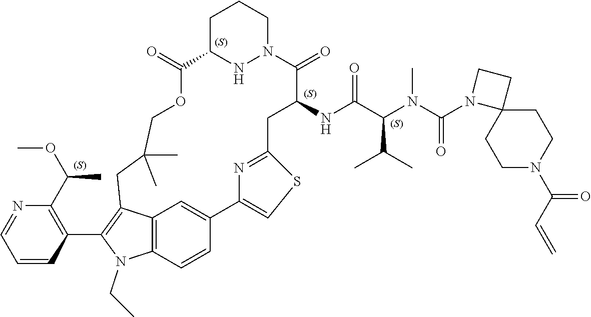 |
| BA679 | 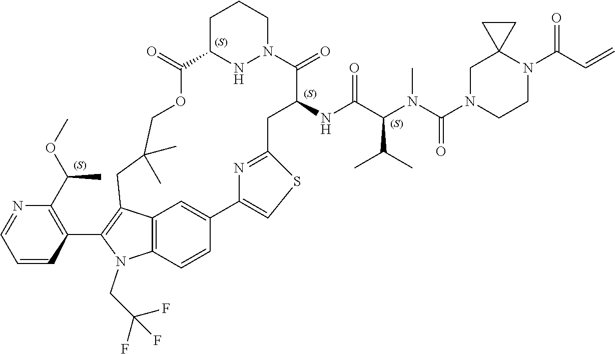 |
| BA680 | 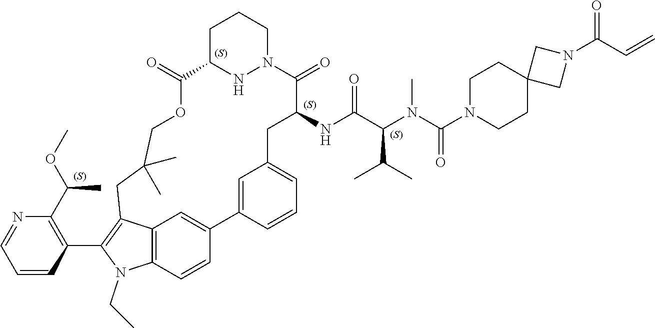 |
| BA681 | 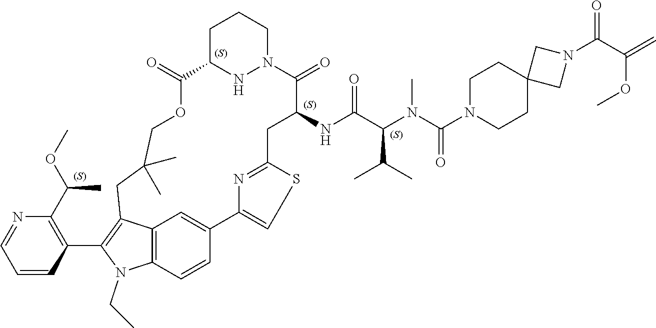 |
| BA682 | 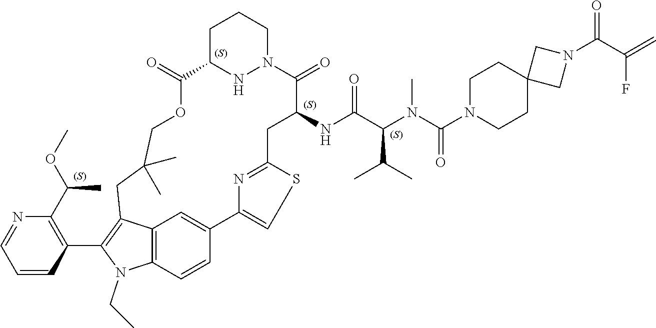 |
| BA683 | 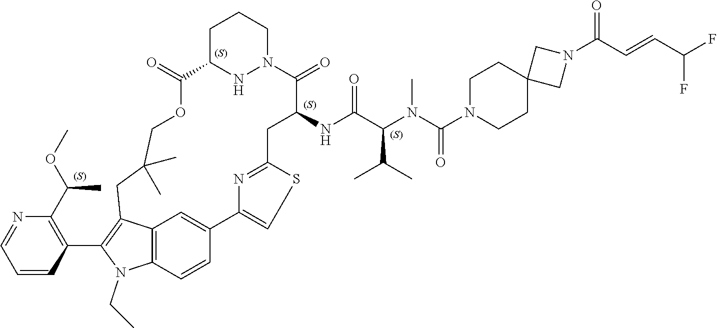 |
| BA684 | 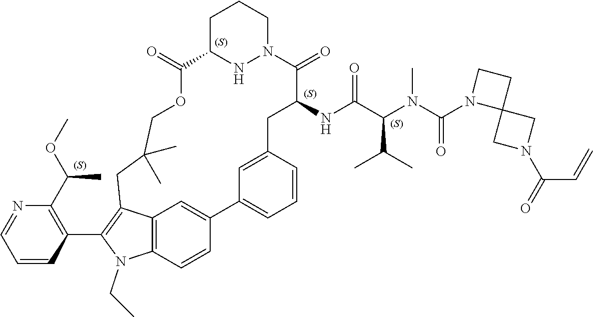 |
| BA685 | 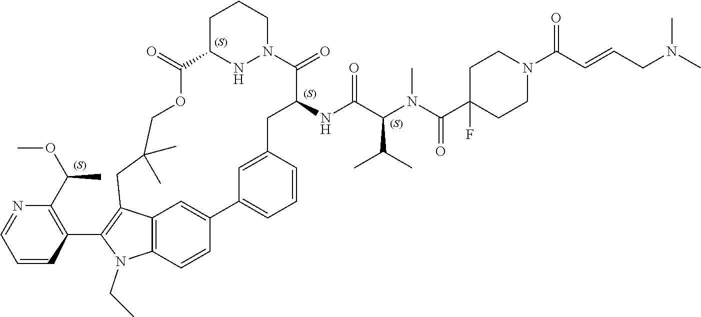 |
| BA686 | 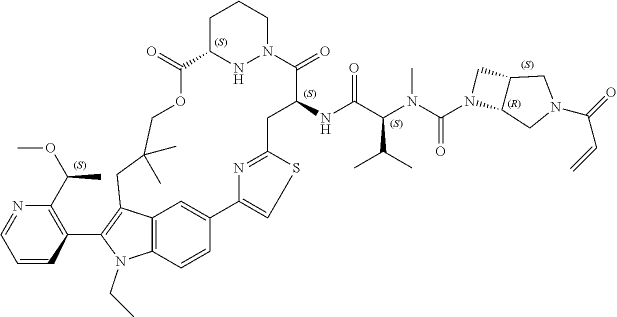 |
| BA687 | 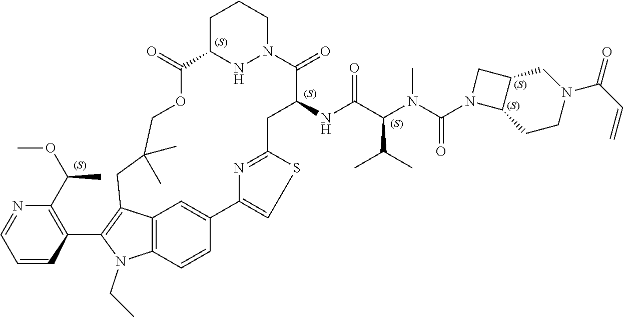 |
| BA688 | 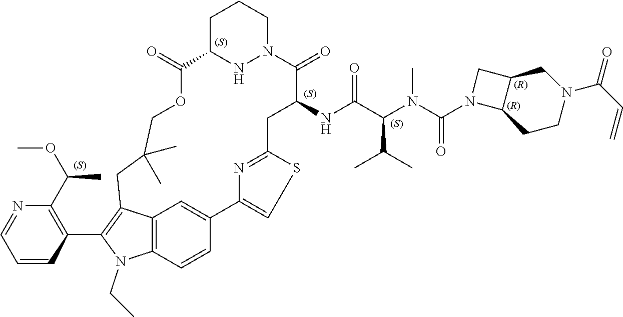 |
| BA689 | 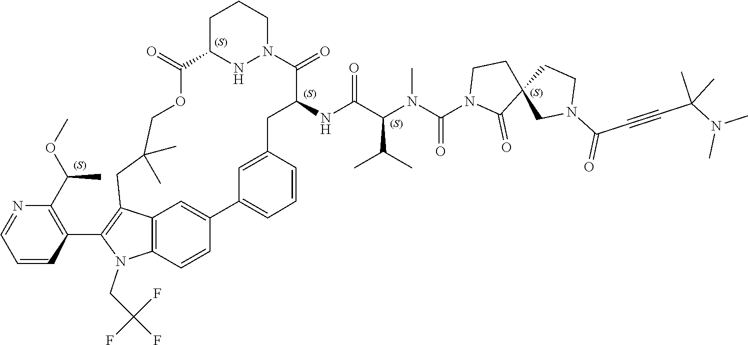 |
| BA690 | 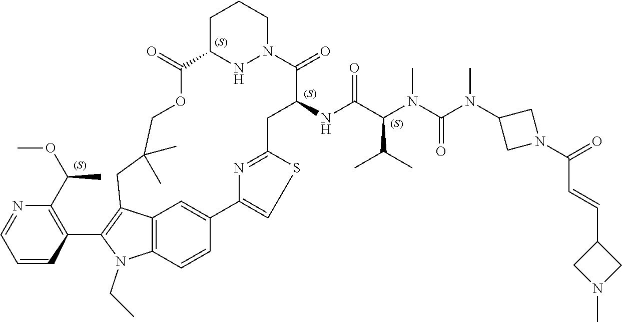 |
| BA691 | 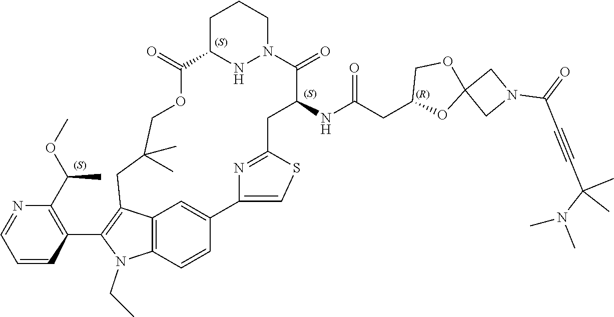 |
| BA692 | 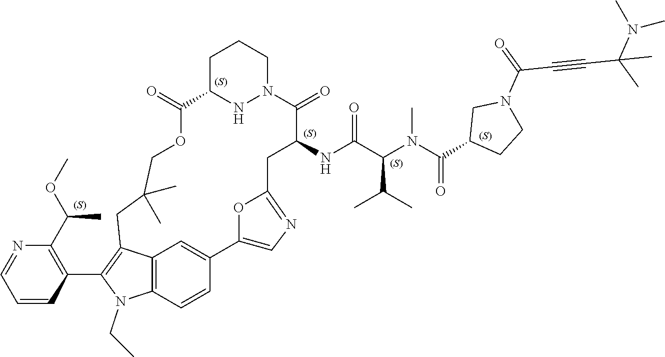 |
| BA693 | 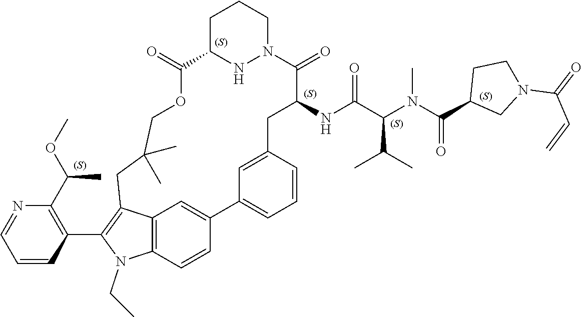 |
| BA694 | 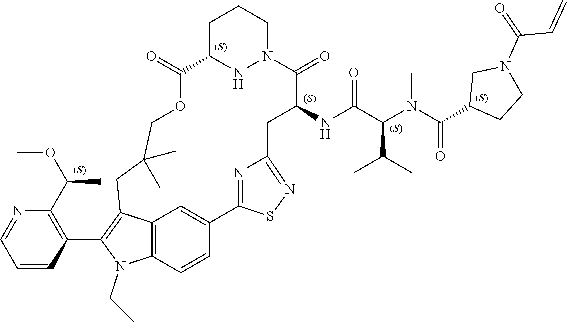 |
| BA695 | 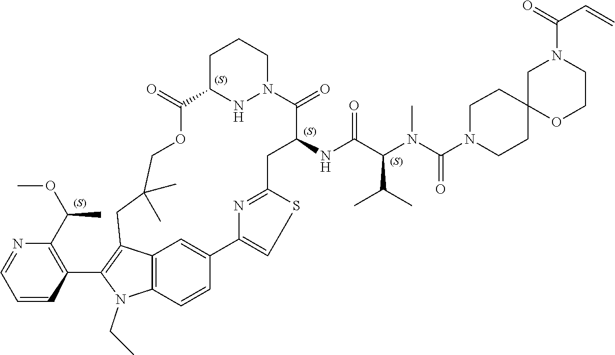 |
| BA696 | 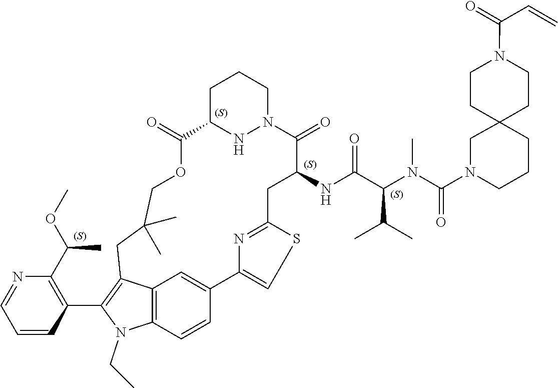 |
| BA697 | 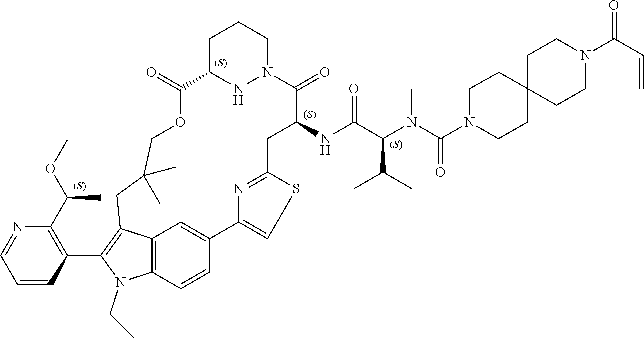 |
| BA698 | 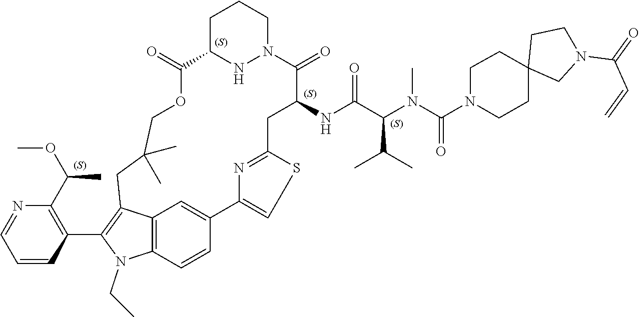 |
| BA699 | 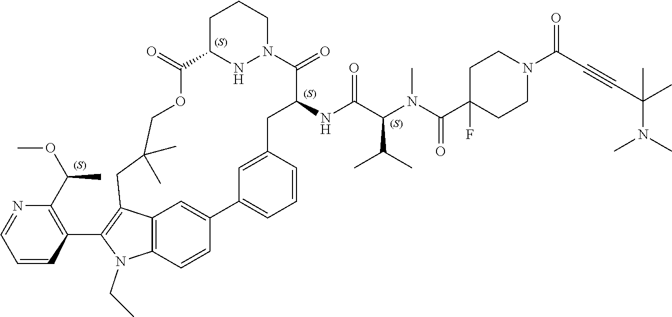 |
| BA700 | 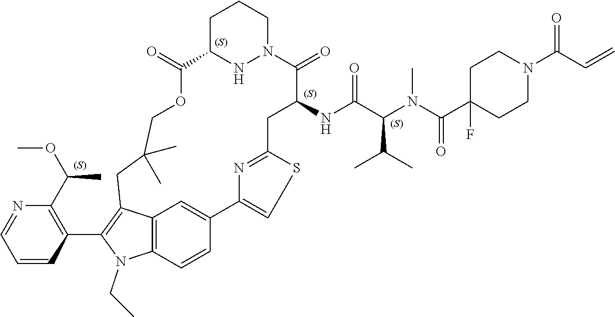 |
| BA701 | 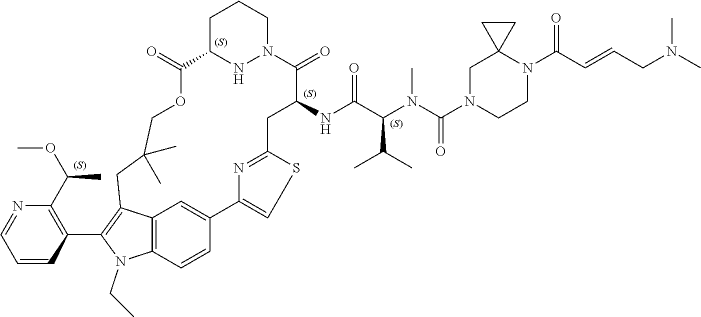 |
| BA702 | 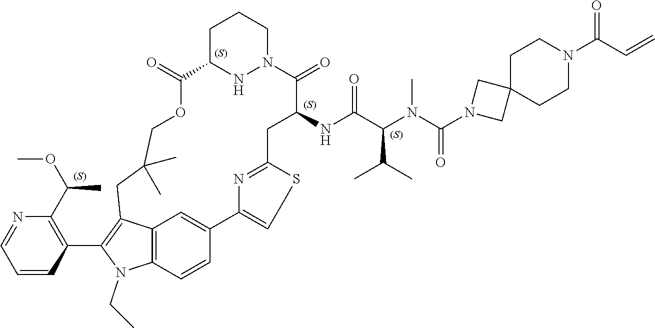 |
| BA703 | 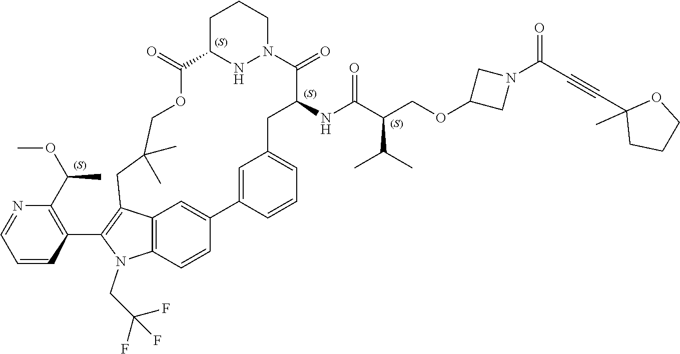 |
| BA704 | 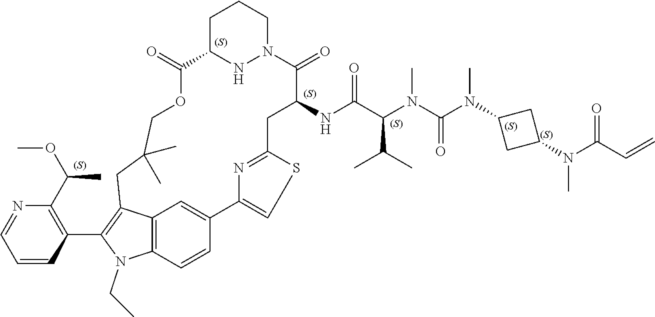 |
| BA705 | 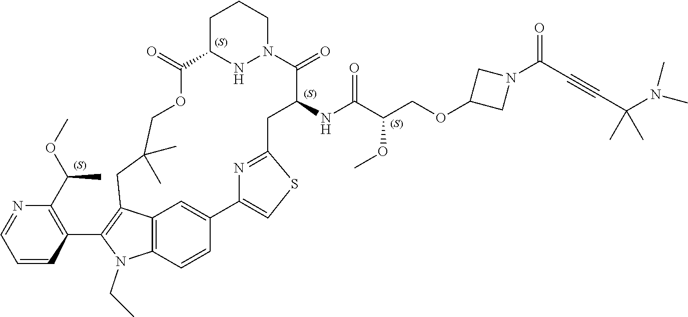 |
| BA706 | 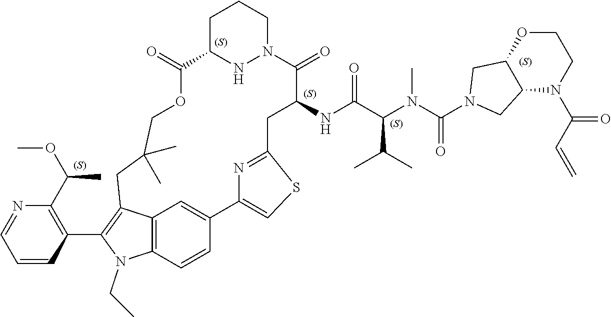 |
| BA707 | 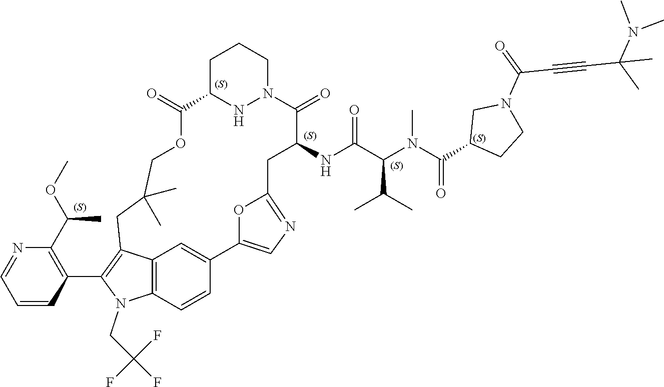 |
| BA708 | 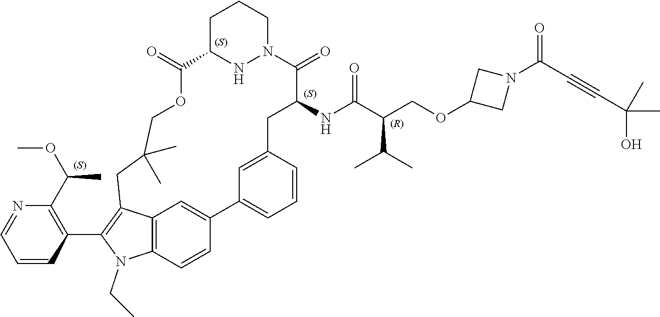 |
| BA709 | 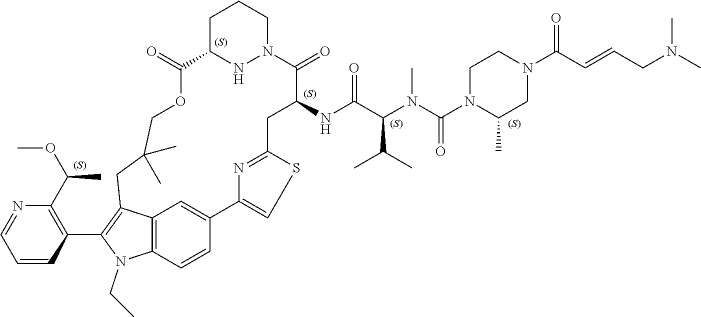 |
| BA710 | 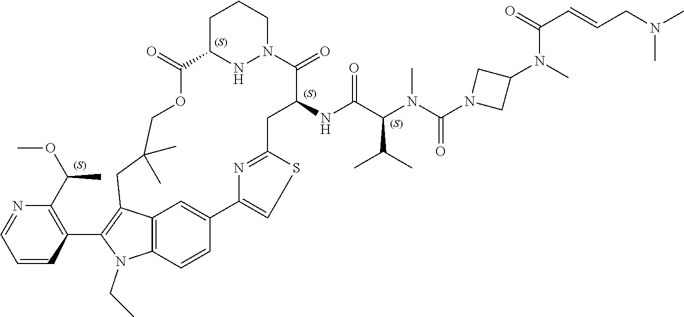 |
| BA711 | 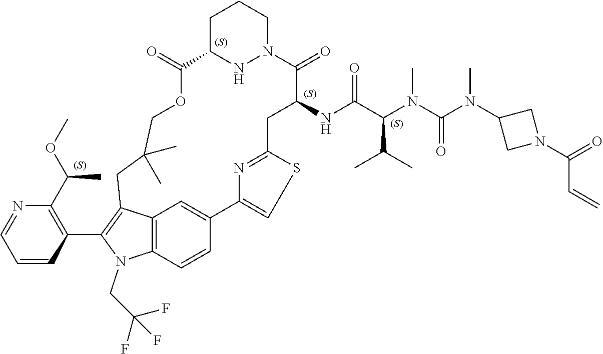 |
| BA712 | 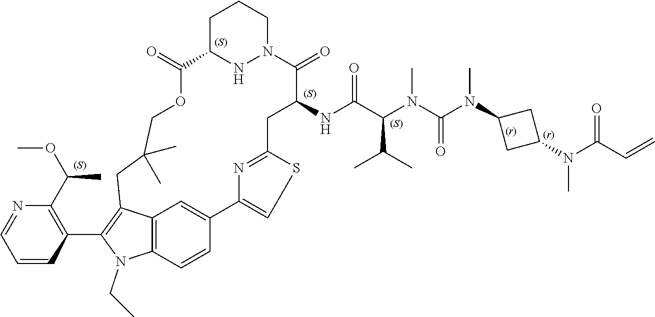 |
| BA713 | 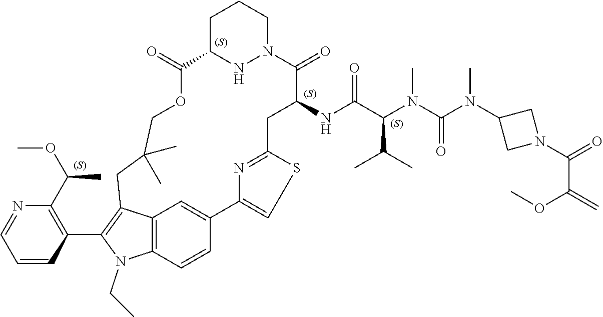 |
| BA714 | 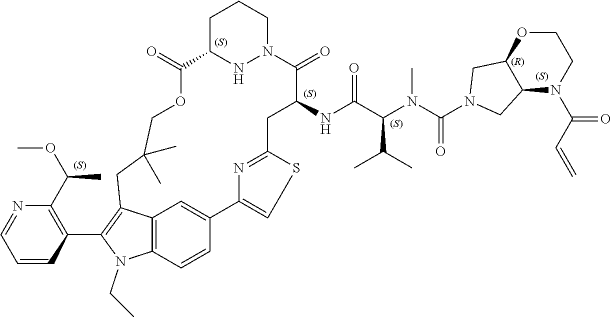 |
| BA715 | 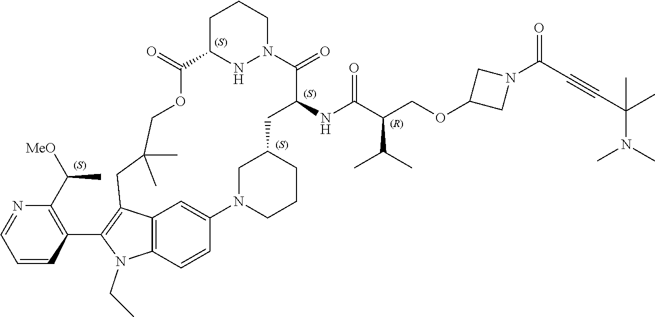 |
| BA716 | 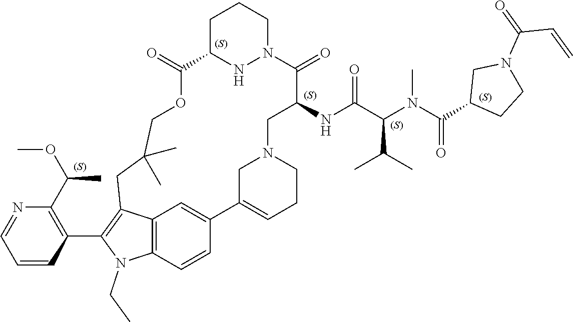 |
| BA717 | 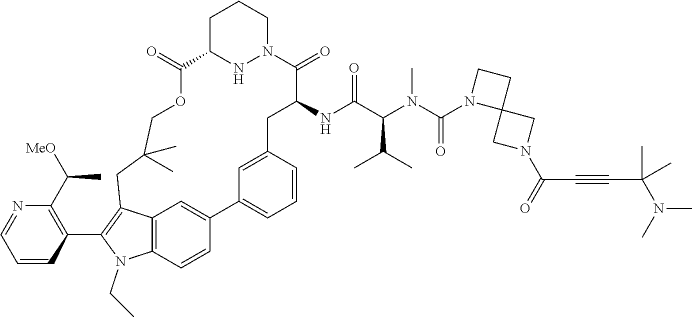 |
| BA718 | 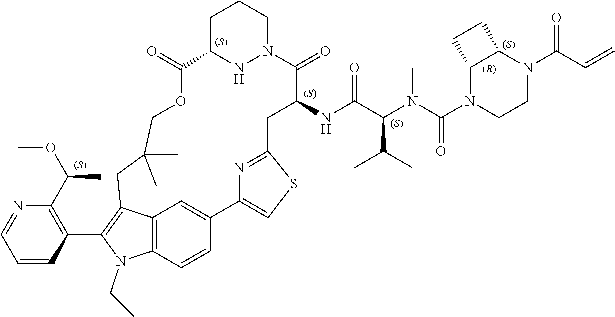 |
| BA719 | 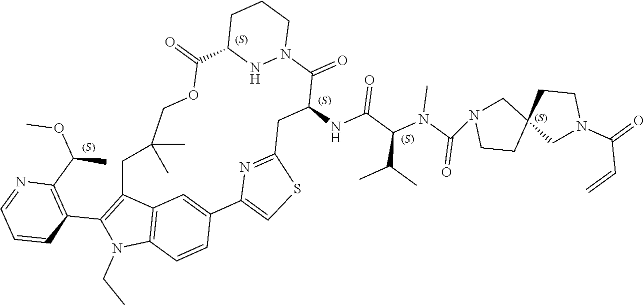 |
| BA720 | 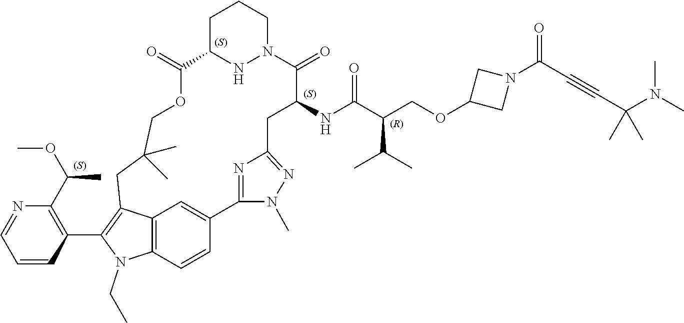 |
| BA721 | 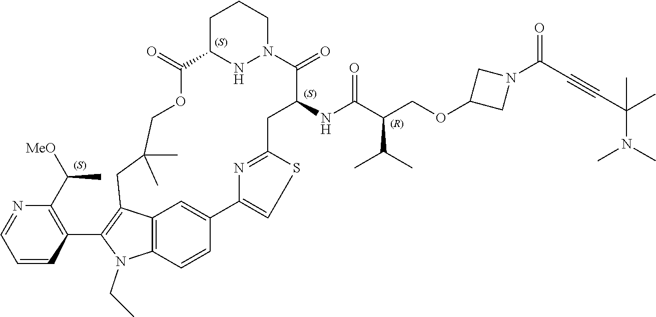 |
| BA722 | 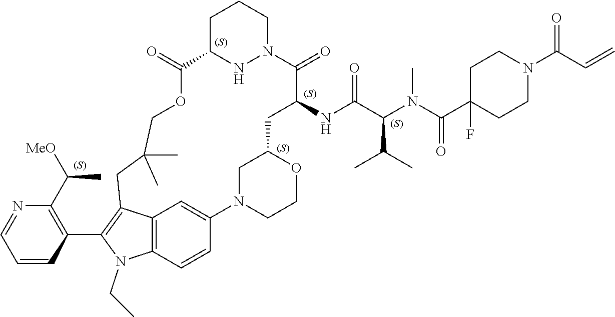 |
| BA723 | 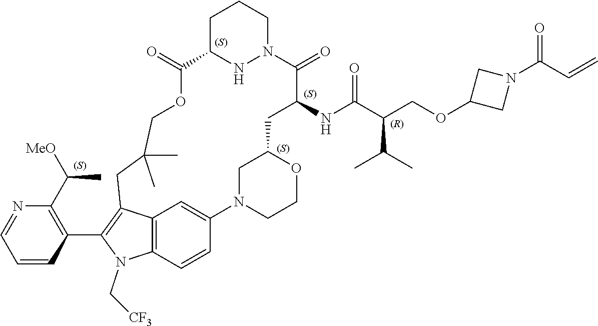 |
| BA724 | 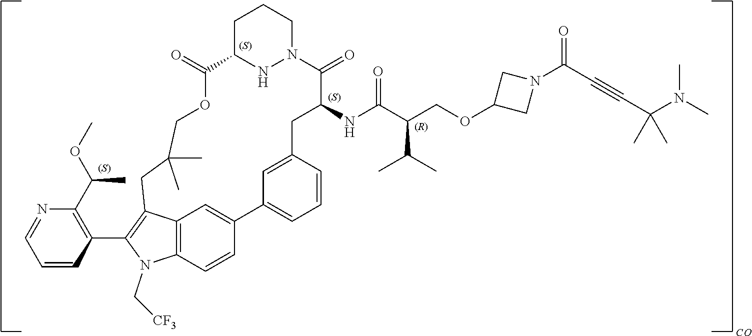 |
| BA725 | 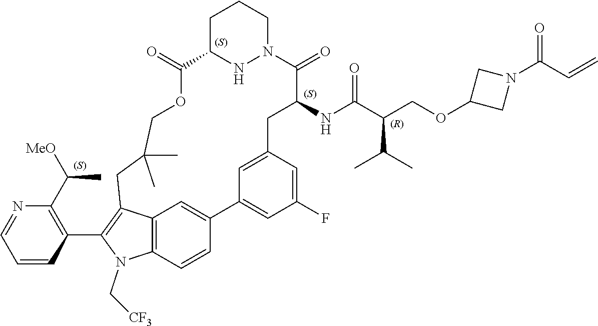 |
| BA726 | 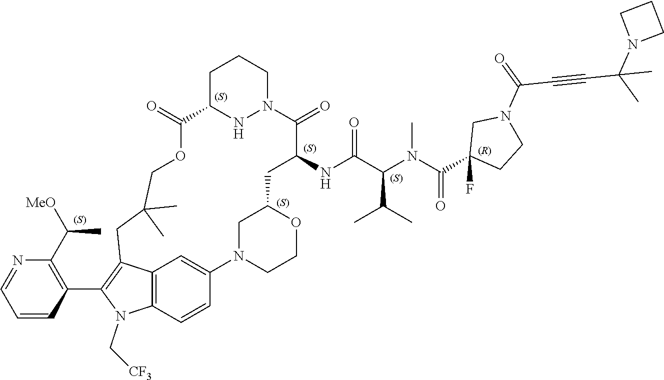 |
| BA727 | 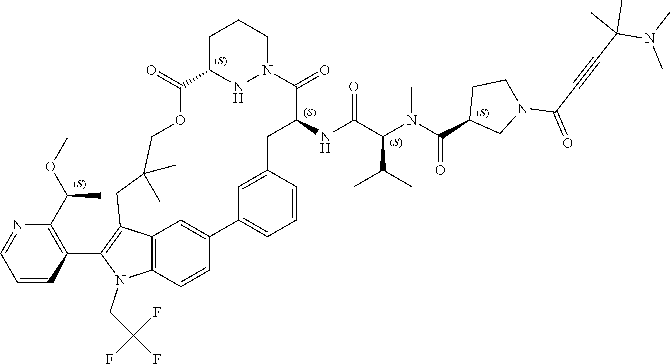 |
| BA728 | 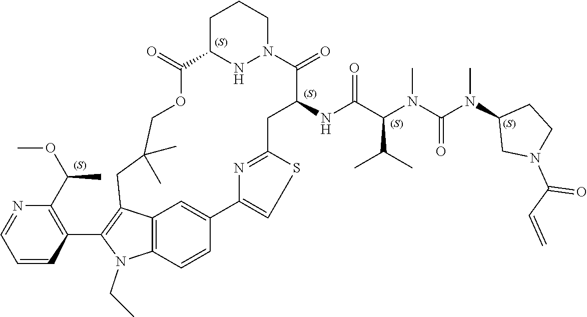 |
| BA729 | 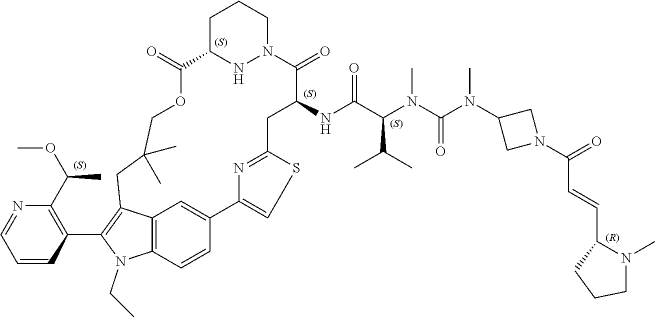 |
| BA730 | 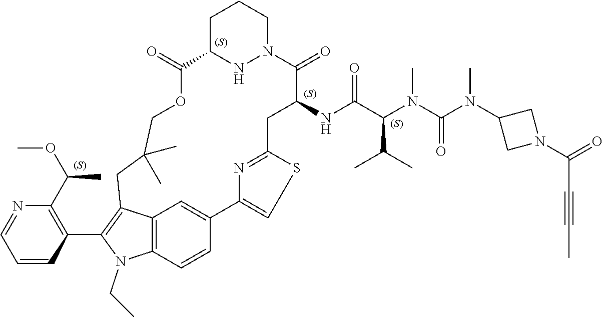 |
| BA731 | 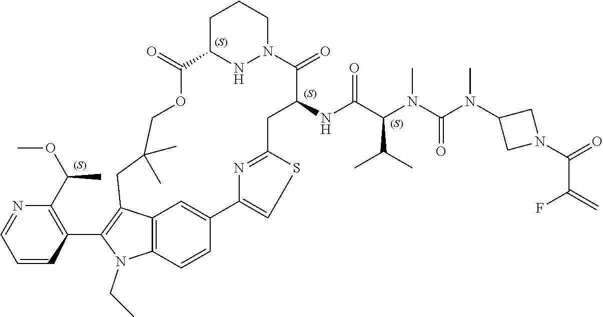 |
| BA732 | 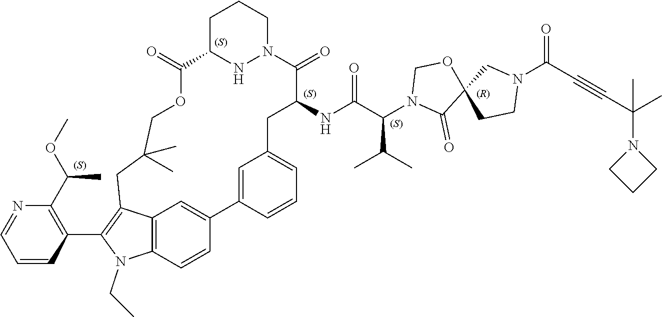 |
| BA733 | 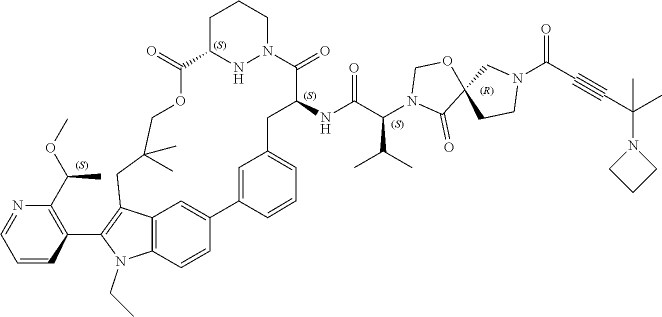 |
| BA734 | 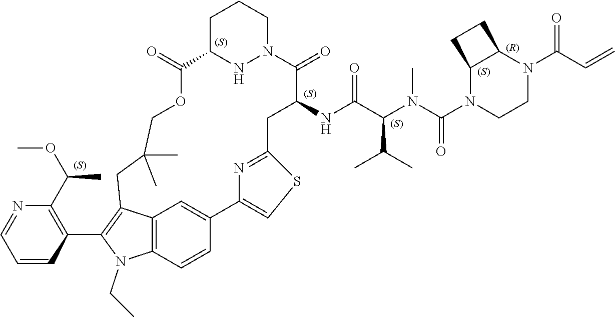 |
| BA735 | 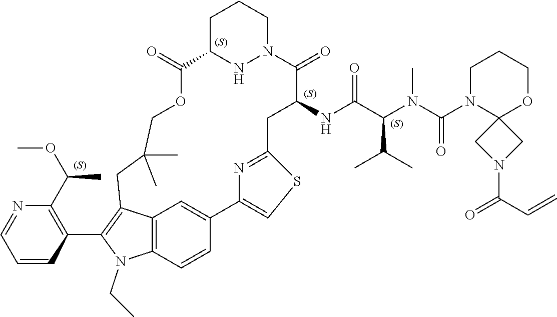 |
| BA736 | 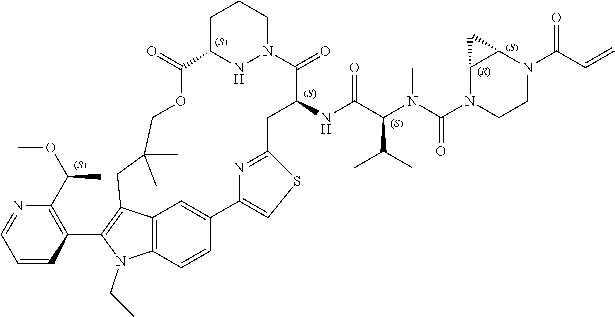 |
| BA737 | 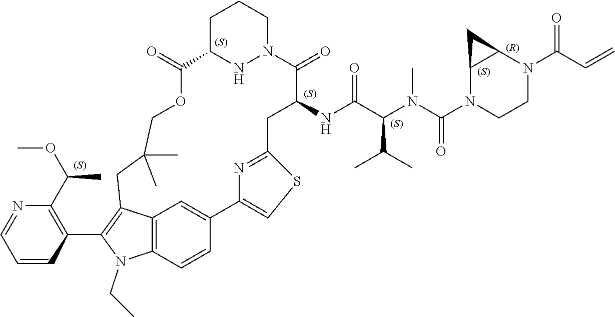 |
| BA738 | 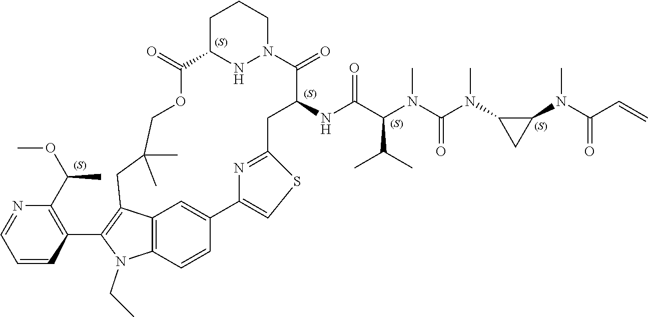 |
| BA739 | 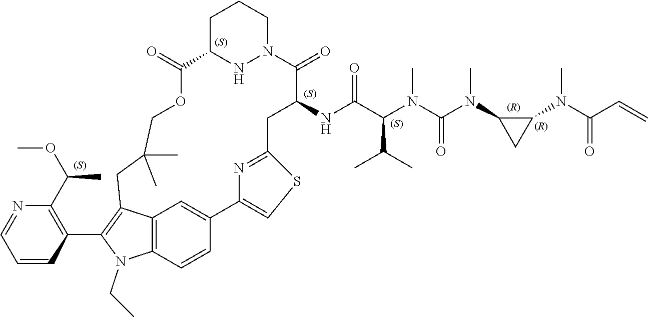 |
| BA740 | 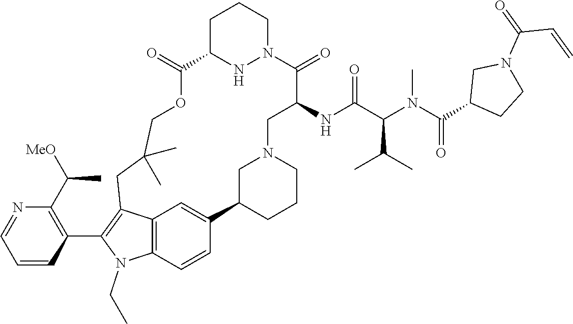 |
| BA741 | 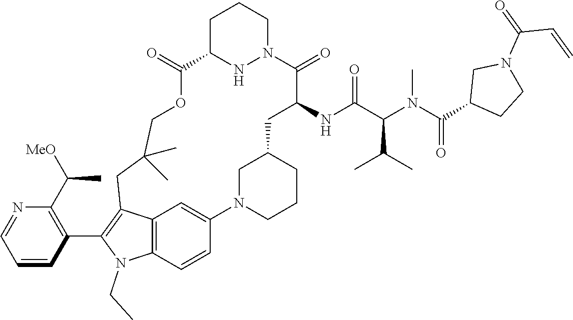 |

Note
that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated. Brackets are to be ignored.
*The activity of this stereoisomer may, in fact, be attributable to the presence of a small amount of the
stereoisomer with the (S) configuration at the —NC(O)—CH(CH₃)₂—N(CH₃)— position.

In some embodiments, a compound of Table B2 is provided, or a pharmaceutically acceptable salt thereof. In some embodiments, the RAS(ON) inhibitor is selected from Table B2, or a pharmaceutically acceptable salt or atropisomer thereof.

TABLE B2
Certain Compounds of the Present Invention

| Ex # | Structure |
|---|---|
| BB1 | 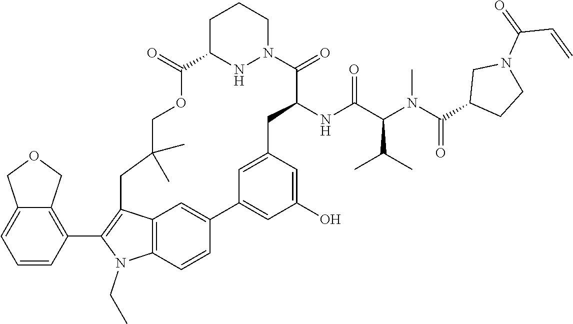 [SMILES: C=CC(=O)N1CC[C@H](C(=O)N(C)[C@H](C(=O)N[C@H]2Cc3cc(O)cc(c3)-c3ccc4c(c3)c(c(-c3cccc5c3COC5)n4CC)CC(C)(C)COC(=O)[C@@H]3CCCN(N3)C2=O)C(C)C)C1] |
| BB2 | 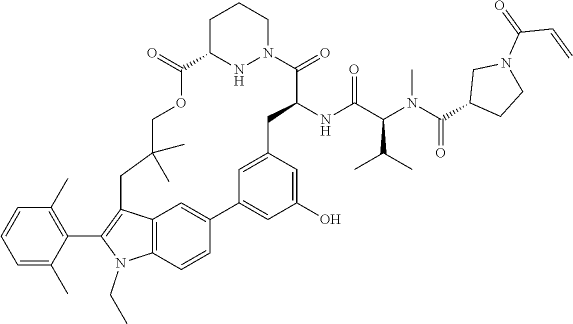 [SMILES: C=CC(=O)N1CC[C@H](C(=O)N(C)[C@H](C(=O)N[C@H]2Cc3cc(O)cc(c3)-c3ccc4c(c3)c(c(-c3c(C)cccc3C)n4CC)CC(C)(C)COC(=O)[C@@H]3CCCN(N3)C2=O)C(C)C)C1] |
| BB3 | 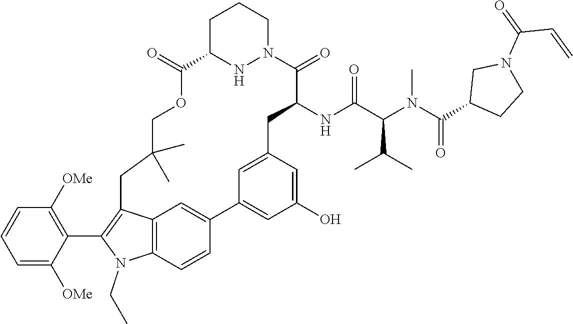 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cccc3OC)n4CC)CC(C)(C)COC(=O)[C@@H]3CCCN(N3)C2=O)C(C)C)C1] |
| BB4 | 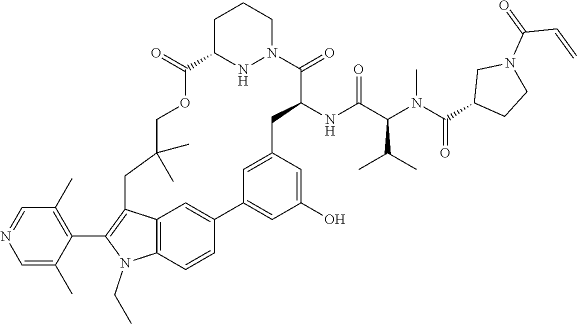 [SMILES: C=CC(=O)N1CC[C@H](C(=O)N(C)[C@H](C(=O)N[C@H]2Cc3cc(O)cc(c3)-c3ccc4c(c3)c(c(-c3c(C)cncc3C)n4CC)CC(C)(C)COC(=O)[C@@H]3CCCN(N3)C2=O)C(C)C)C1] |
| BB5 | 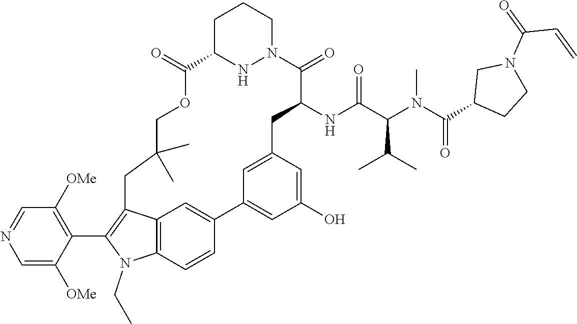 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cncc3OC)n4CC)CC(C)(C)COC(=O)[C@@H]3CCCN(N3)C2=O)C(C)C)C1] |
| BB6 | 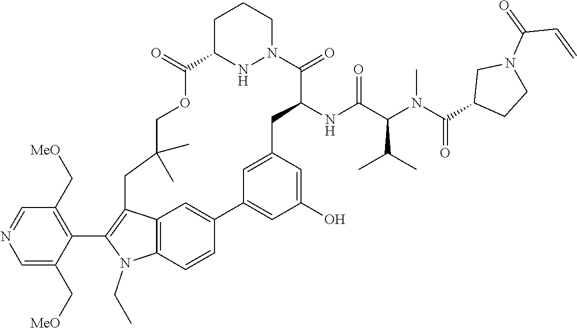 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cncc3COC)n4CC)CC(C)(C)COC(=O)[C@@H]3CCCN(N3)C2=O)C(C)C)C1] |
| BB7 | 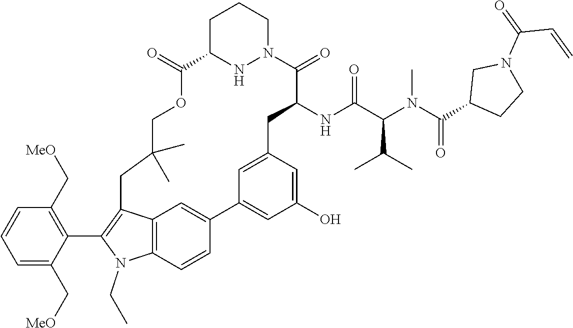 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cccc3COC)n4CC)CC(C)(C)COC(=O)[C@@H]3CCCN(N3)C2=O)C(C)C)C1] |
| BB11 | 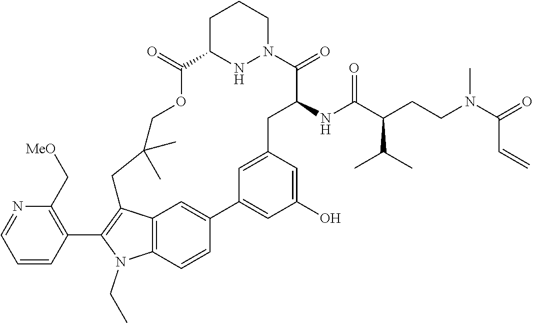 [SMILES: C=CC(=O)N(C)CC[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| BB12 | 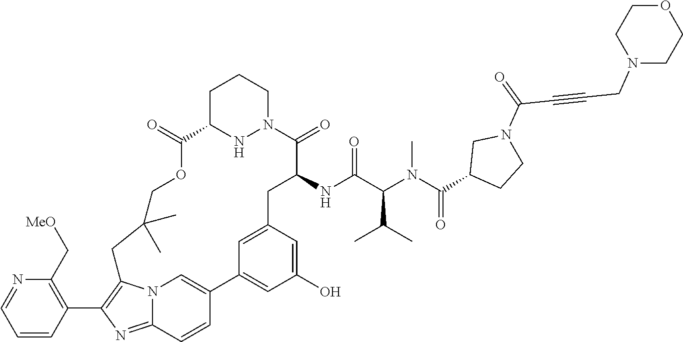 [SMILES: COCc1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CCN2CCOCC2)C1)Cc1cc(O)cc-3c1] |
| BB13 | 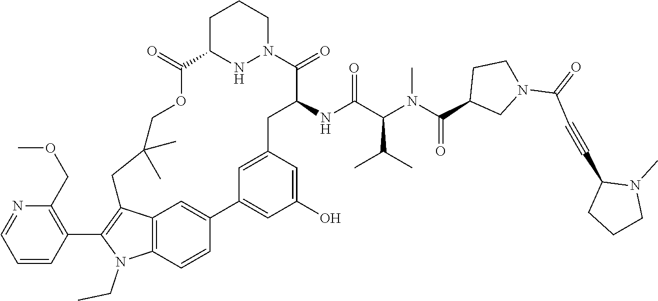 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| BB18 | 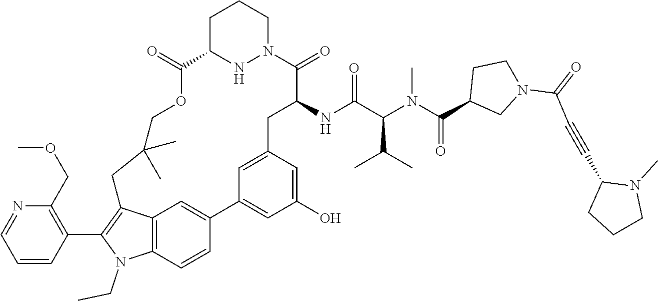 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H]3CCCN3C)C1)C(=O)N1CCC[C@H](N1)C(=O)OCC(C)(C)C2] |
| BB21 | 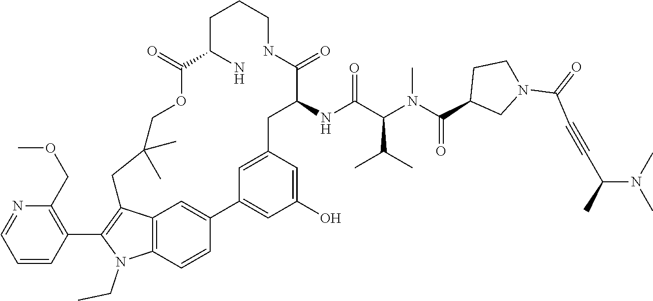 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H](C)N(C)C)C1)C(=O)NCCC[C@H](N)C(=O)OCC(C)(C)C2] |
| BB22 | 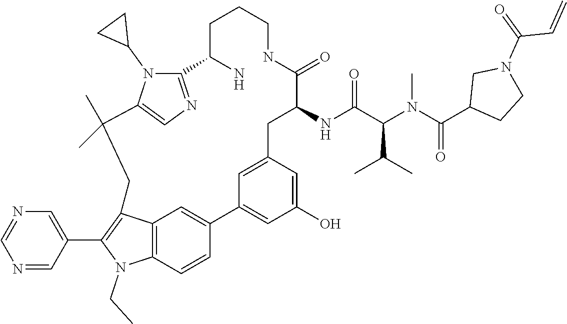 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)c3cnc(n3C3CC3)[C@@H](N)CCCNC2=O)C(C)C)C1] |
| BB25 | 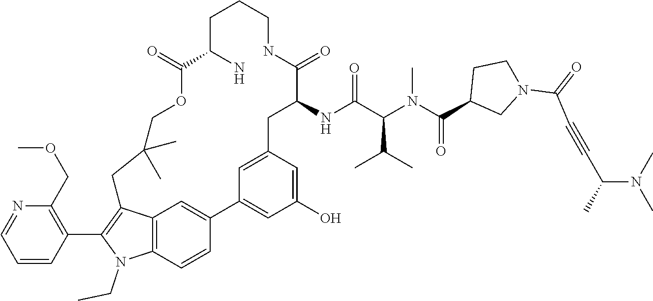 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H](C)N(C)C)C1)C(=O)NCCC[C@H](N)C(=O)OCC(C)(C)C2] |
| BB27 | 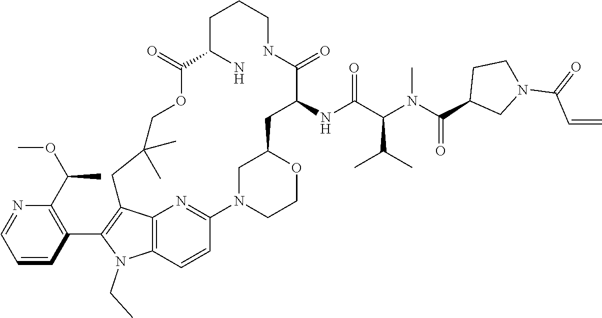 |
| BB28 | 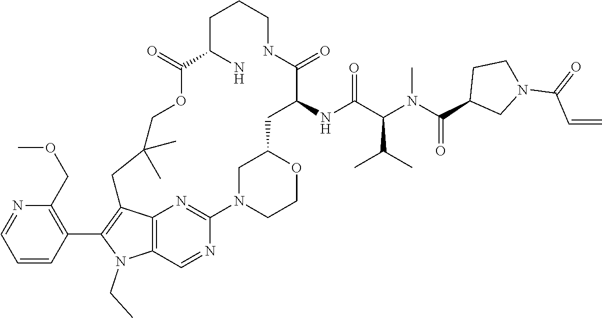 [SMILES: C=CC(=O)N1CC[C@H](C(=O)N(C)[C@H](C(=O)N[C@H]2C[C@H]3CN(CCO3)c3ncc4c(n3)c(c(-c3cccnc3COC)n4CC)CC(C)(C)COC(=O)[C@@H](N)CCCNC2=O)C(C)C)C1] |
| BB29 | 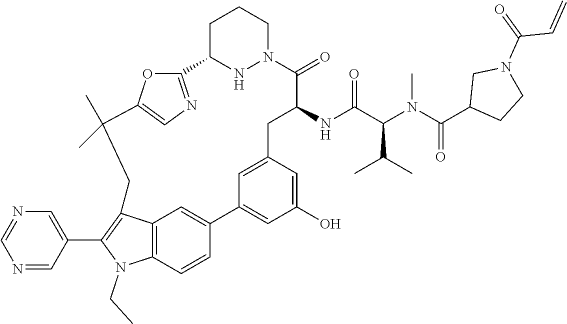 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)C3=CN=C(O3)[C@@H]3CCCN(N3)C2=O)C(C)C)C1] |
| BB30 | 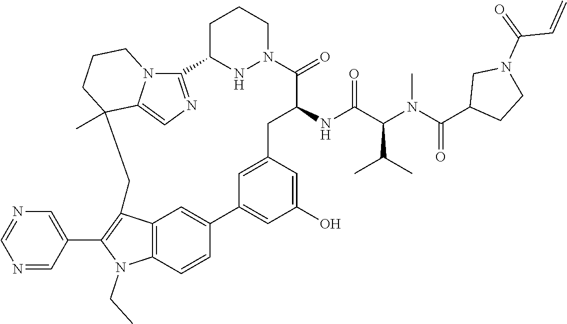 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC3(C)CCCn4c3cnc4[C@@H]3CCCN(N3)C2=O)C(C)C)C1] |
| BB32 | 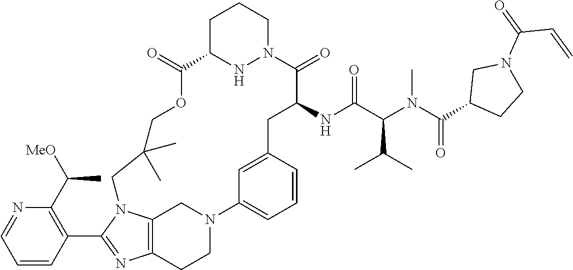 [SMILES: C=CC(=O)N1CC[C@H](C(=O)N(C)[C@H](C(=O)N[C@H]2Cc3cccc(c3)N3CCc4nc(-c5cccnc5[C@H](C)OC)n(c4C3)CC(C)(C)COC(=O)[C@@H]3CCCN(N3)C2=O)C(C)C)C1] |
| BB34 | 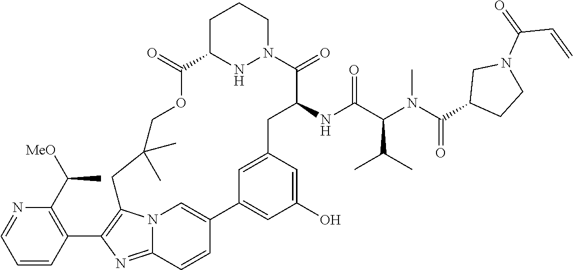 [SMILES: C=CC(=O)N1CC[C@H](C(=O)N(C)[C@H](C(=O)N[C@H]2Cc3cc(O)cc(c3)-c3ccc4nc(-c5cccnc5[C@H](C)OC)c(n4c3)CC(C)(C)COC(=O)[C@@H]3CCCN(N3)C2=O)C(C)C)C1] |
| BB38 | 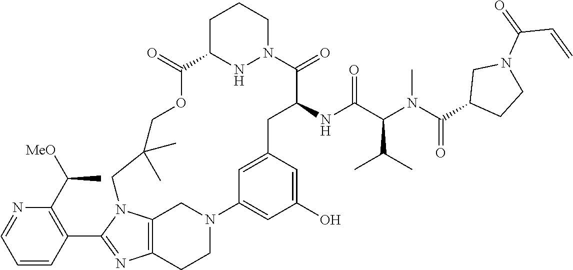 [SMILES: C=CC(=O)N1CC[C@H](C(=O)N(C)[C@H](C(=O)N[C@H]2Cc3cc(O)cc(c3)N3CCc4nc(-c5cccnc5[C@H](C)OC)n(c4C3)CC(C)(C)COC(=O)[C@@H]3CCCN(N3)C2=O)C(C)C)C1] |
| BB47 | 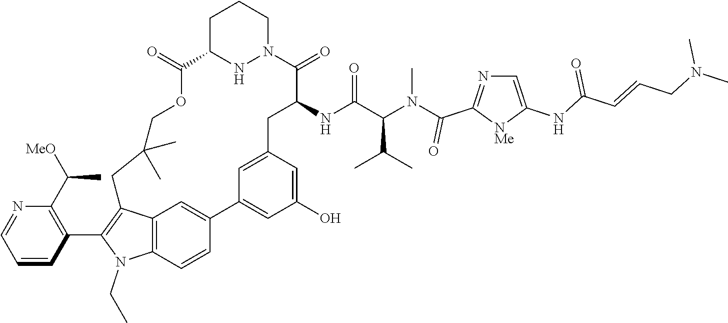 |
| BB64 | 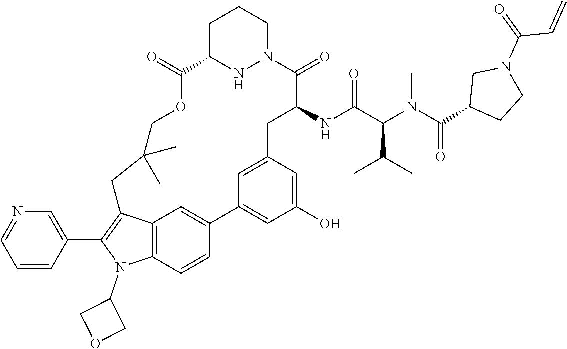 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4C3COC3)CC(C)(C)COC(=O)[C@@H]3CCCN(N3)C2=O)C(C)C)C1] |
| BB65 | 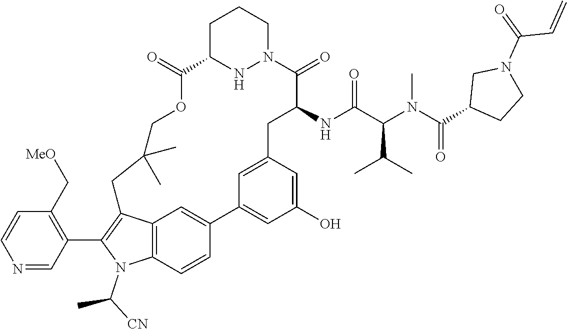 [SMILES: C=CC(=O)N1CC[C@H](C(=O)N(C)[C@H](C(=O)N[C@H]2Cc3cc(O)cc(c3)-c3ccc4c(c3)c(c(-c3cnccc3COC)n4[C@H](C)C#N)CC(C)(C)COC(=O)[C@@H]3CCCN(N3)C2=O)C(C)C)C1] |
| BB66 | 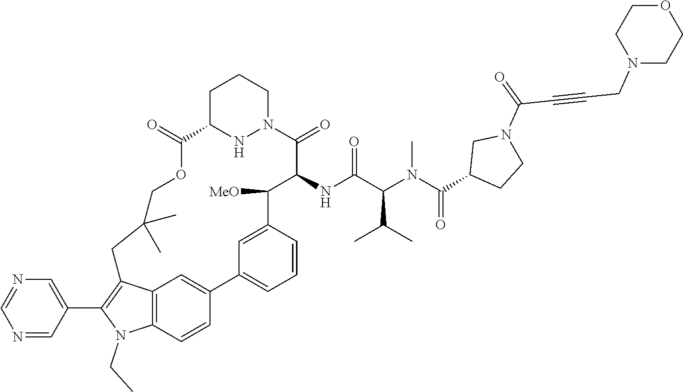 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB70 | 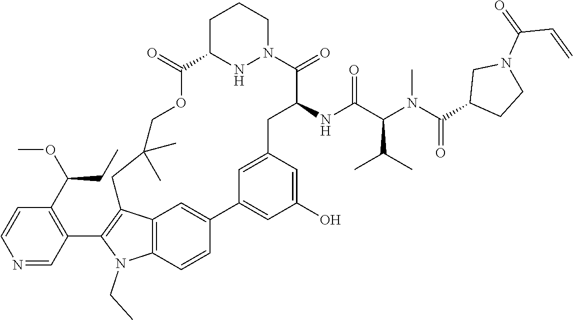 [SMILES: C=CC(=O)N1CC[C@H](C(=O)N(C)[C@H](C(=O)N[C@H]2Cc3cc(O)cc(c3)-c3ccc4c(c3)c(c(-c3cnccc3[C@H](CC)OC)n4CC)CC(C)(C)COC(=O)[C@@H]3CCCN(N3)C2=O)C(C)C)C1] |
| BB73 | 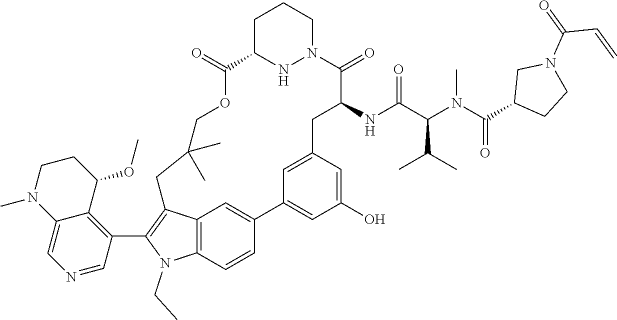 [SMILES: C=CC(=O)N1CC[C@H](C(=O)N(C)[C@H](C(=O)N[C@H]2Cc3cc(O)cc(c3)-c3ccc4c(c3)c(c(-c3cncc5c3[C@@H](OC)CCN5C)n4CC)CC(C)(C)COC(=O)[C@@H]3CCCN(N3)C2=O)C(C)C)C1] |
| BB74 | 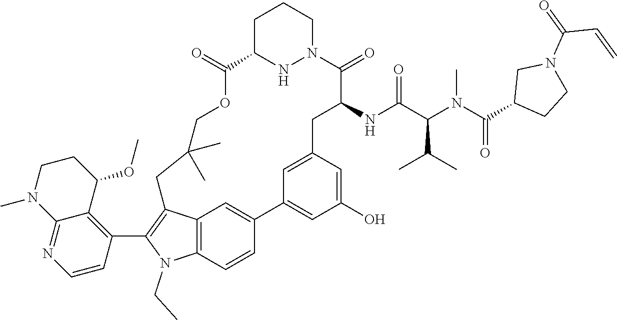 [SMILES: C=CC(=O)N1CC[C@H](C(=O)N(C)[C@H](C(=O)N[C@H]2Cc3cc(O)cc(c3)-c3ccc4c(c3)c(c(-c3ccnc5c3[C@@H](OC)CCN5C)n4CC)CC(C)(C)COC(=O)[C@@H]3CCCN(N3)C2=O)C(C)C)C1] |
| BB75 | 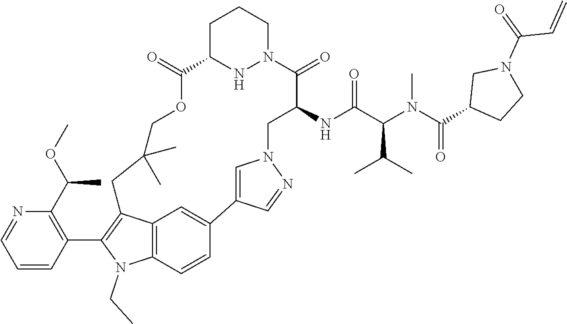 [SMILES: C=CC(=O)N1CC[C@H](C(=O)N(C)[C@H](C(=O)N[C@H]2Cn3cc(cn3)-c3ccc4c(c3)c(c(-c3cccnc3[C@H](C)OC)n4CC)CC(C)(C)COC(=O)[C@@H]3CCCN(N3)C2=O)C(C)C)C1] |
| BB76 | 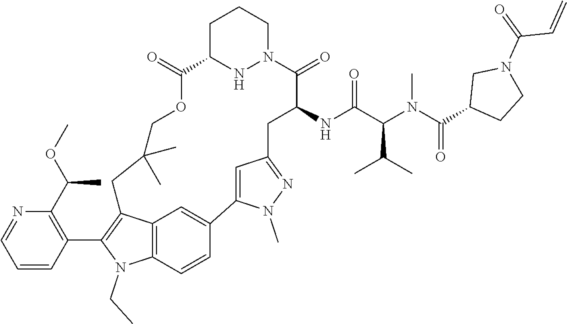 [SMILES: C=CC(=O)N1CC[C@H](C(=O)N(C)[C@H](C(=O)N[C@H]2Cc3cc(n(C)n3)-c3ccc4c(c3)c(c(-c3cccnc3[C@H](C)OC)n4CC)CC(C)(C)COC(=O)[C@@H]3CCCN(N3)C2=O)C(C)C)C1] |
| BB77 | 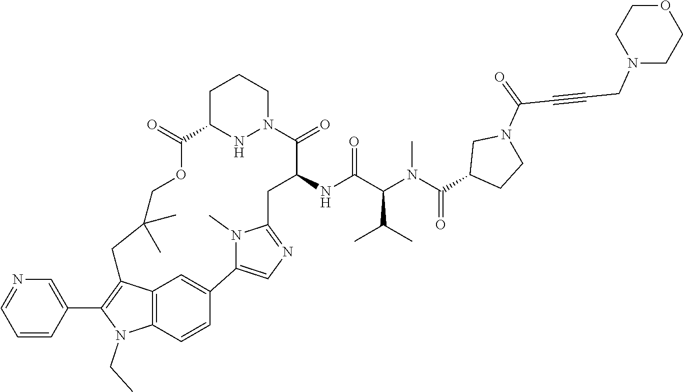 [SMILES: CCn1c(-c2cccnc2)c2c3cc(ccc31)-c1cnc(n1C)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB81 | 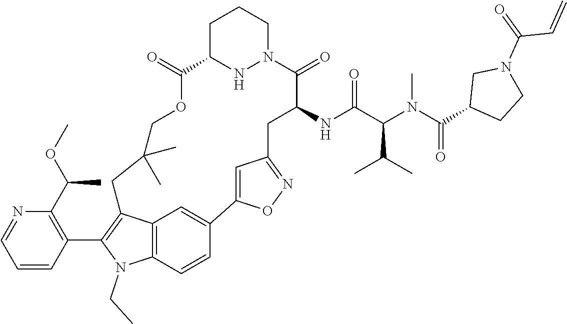 [SMILES: C=CC(=O)N1CC[C@H](C(=O)N(C)[C@H](C(=O)N[C@H]2Cc3cc(on3)-c3ccc4c(c3)c(c(-c3cccnc3[C@H](C)OC)n4CC)CC(C)(C)COC(=O)[C@@H]3CCCN(N3)C2=O)C(C)C)C1] |
| BB83 | 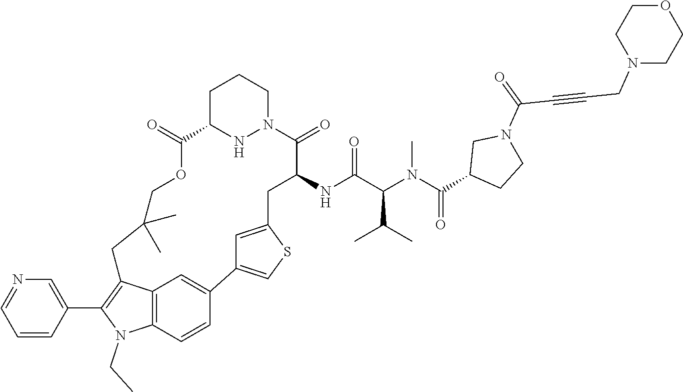 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB85 | 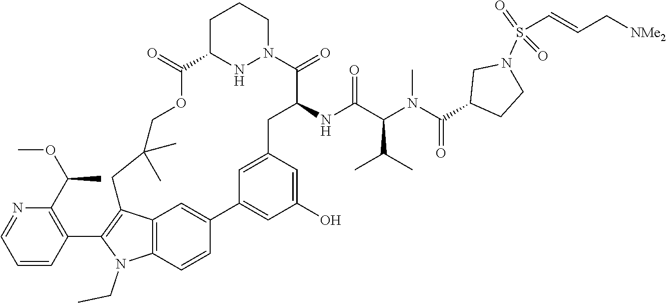 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)/C=C/CN(C)C)C1)C(=O)N1CCC[C@H](N1)C(=O)OCC(C)(C)C2] |
| BB86 | 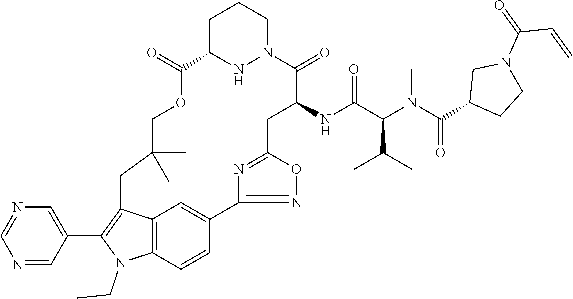 [SMILES: C=CC(=O)N1CC[C@H](C(=O)N(C)[C@H](C(=O)N[C@H]2Cc3nc(no3)-c3ccc4c(c3)c(c(-c3cncnc3)n4CC)CC(C)(C)COC(=O)[C@@H]3CCCN(N3)C2=O)C(C)C)C1] |
| BB87 | 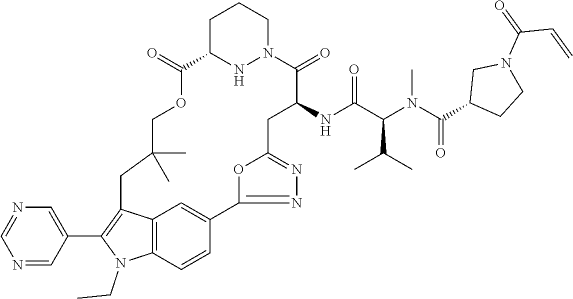 [SMILES: C=CC(=O)N1CC[C@H](C(=O)N(C)[C@H](C(=O)N[C@H]2Cc3nnc(o3)-c3ccc4c(c3)c(c(-c3cncnc3)n4CC)CC(C)(C)COC(=O)[C@@H]3CCCN(N3)C2=O)C(C)C)C1] |
| BB88 | 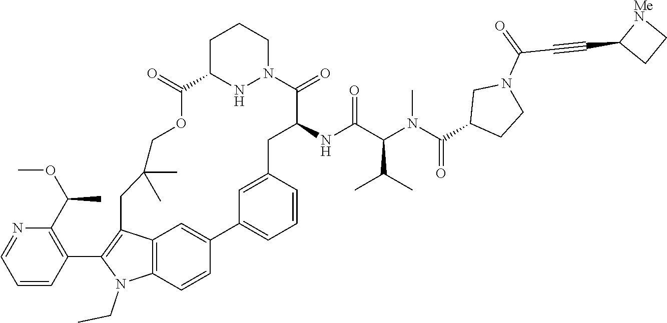 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| BB89 | 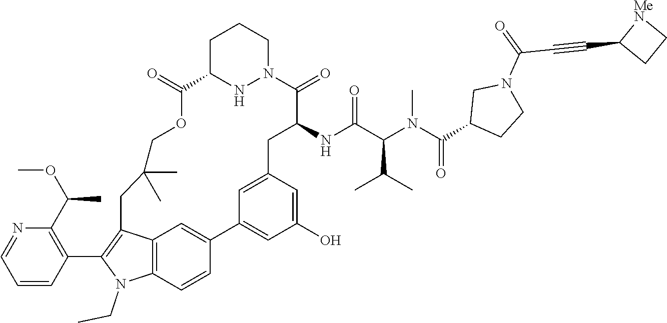 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| BB90 | 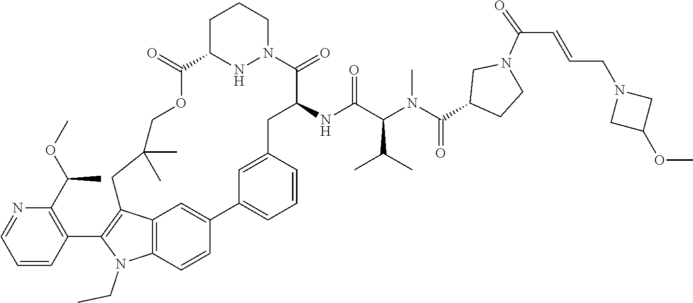 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)/C=C/CN3CC(OC)C3)C1)C(=O)N1CCC[C@H](N1)C(=O)OCC(C)(C)C2] |
| BB91 | 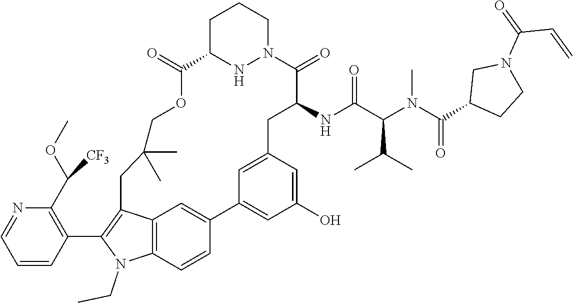 [SMILES: C=CC(=O)N1CC[C@H](C(=O)N(C)[C@H](C(=O)N[C@H]2Cc3cc(O)cc(c3)-c3ccc4c(c3)c(c(-c3cccnc3[C@@H](OC)C(F)(F)F)n4CC)CC(C)(C)COC(=O)[C@@H]3CCCN(N3)C2=O)C(C)C)C1] |
| BB96 | 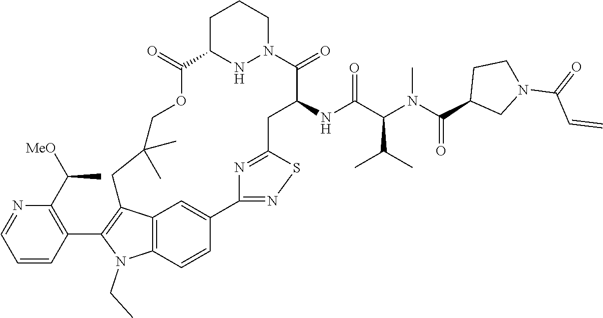 [SMILES: C=CC(=O)N1CC[C@H](C(=O)N(C)[C@H](C(=O)N[C@H]2Cc3nc(ns3)-c3ccc4c(c3)c(c(-c3cccnc3[C@H](C)OC)n4CC)CC(C)(C)COC(=O)[C@@H]3CCCN(N3)C2=O)C(C)C)C1] |
| BB97 | 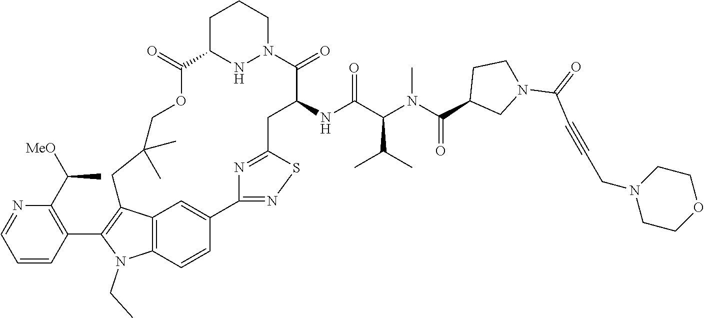 [SMILES: CCn1c(-c2cccnc2[C@H](C)OC)c2c3cc(ccc31)-c1nsc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB102 | 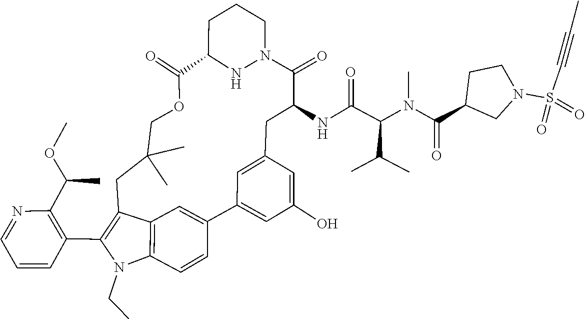 [SMILES: CC#CS(=O)(=O)N1CC[C@H](C(=O)N(C)[C@H](C(=O)N[C@H]2Cc3cc(O)cc(c3)-c3ccc4c(c3)c(c(-c3cccnc3[C@H](C)OC)n4CC)CC(C)(C)COC(=O)[C@@H]3CCCN(N3)C2=O)C(C)C)C1] |
| BB103 | 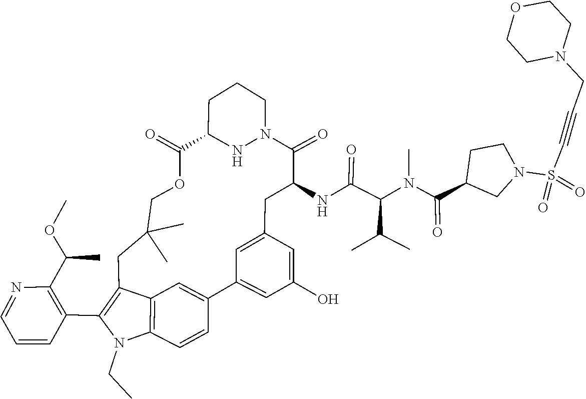 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)C#CCN3CCOCC3)C1)C(=O)N1CCC[C@H](N1)C(=O)OCC(C)(C)C2] |
| BB104 | 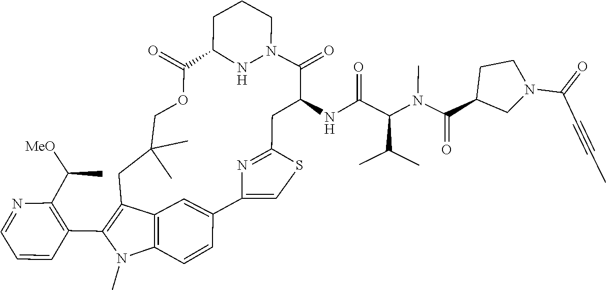 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C)CC(C)(C)COC(=O)[C@@H]3CCCN(N3)C2=O)C(C)C)C1] |
| BB106 | 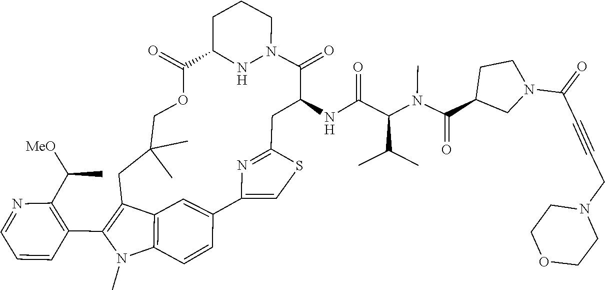 [SMILES: CO[C@@H](C)c1ncccc1-c1c2c3cc(ccc3n1C)-c1csc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB107 | 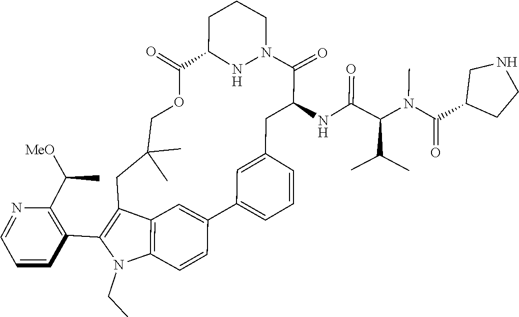 |
| BB109 | 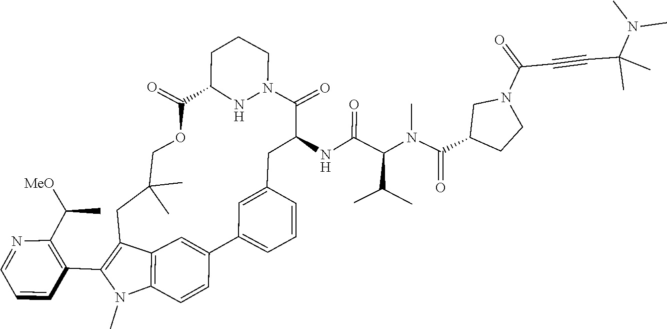 |
| BB111 | 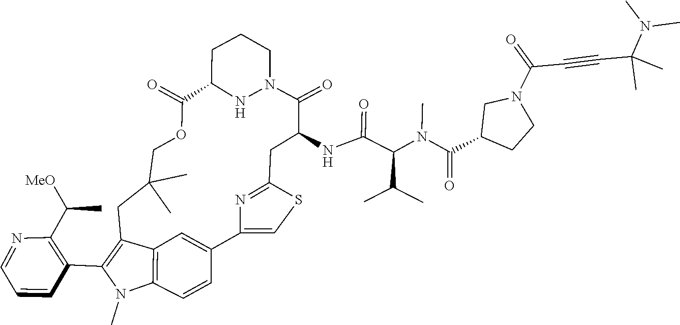 |
| BB112 | 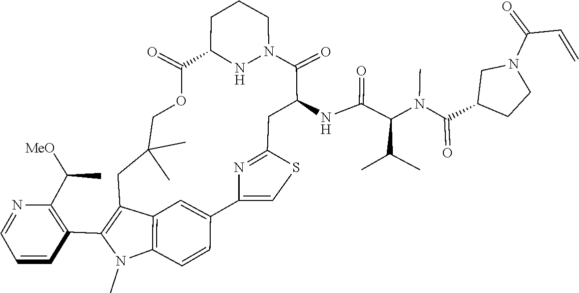 |
| BB113 | 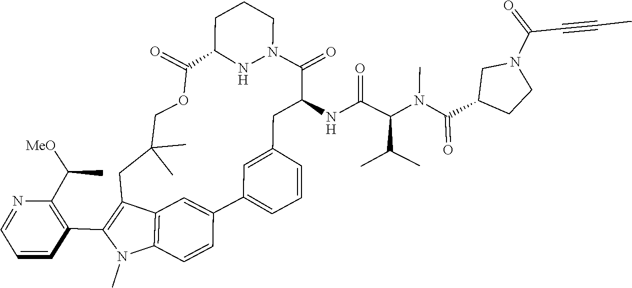 |
| BB115 | 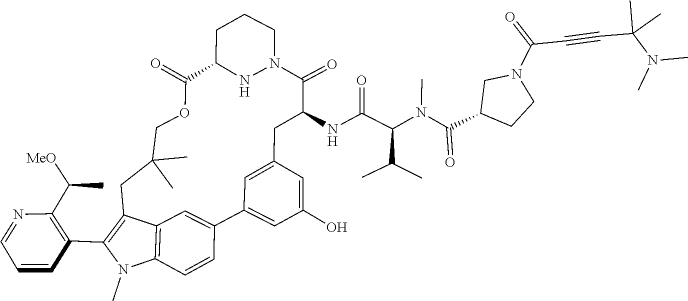 |
| BB116 | 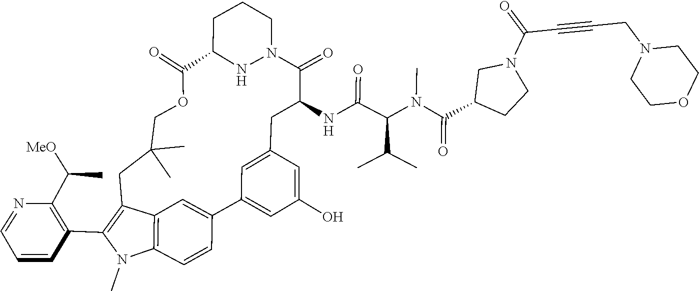 |
| BB117 | 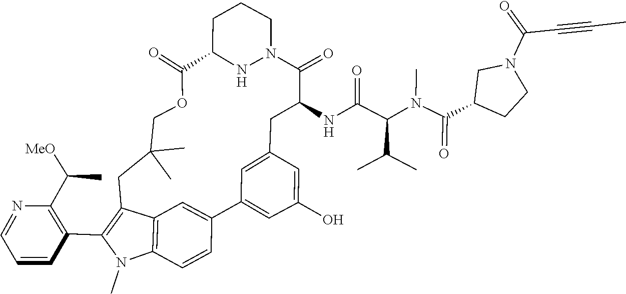 |
| BB118 | 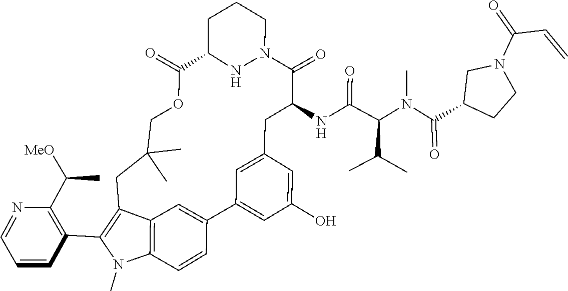 |
| BB119 | 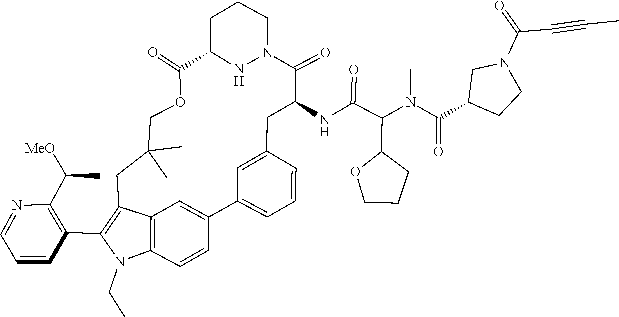 |
| BB120 | 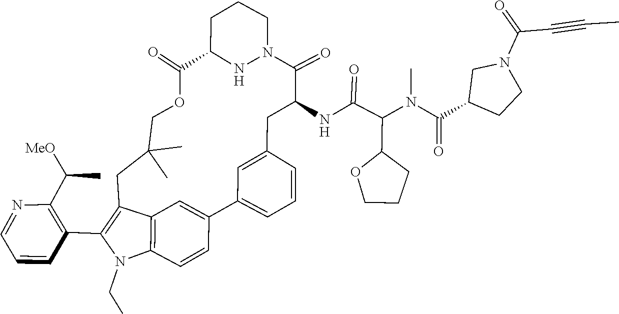 |
| BB121 | 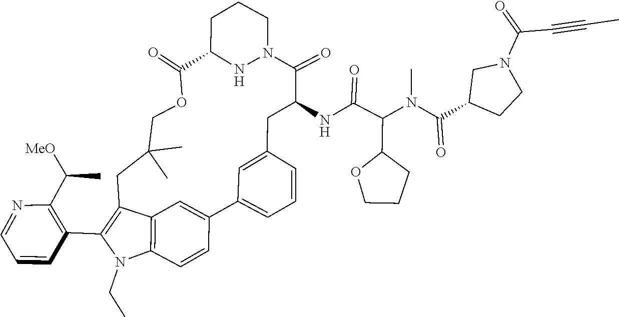 |
| BB122 | 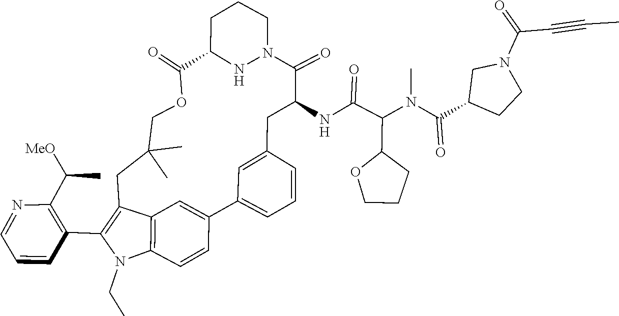 |
| BB123 | 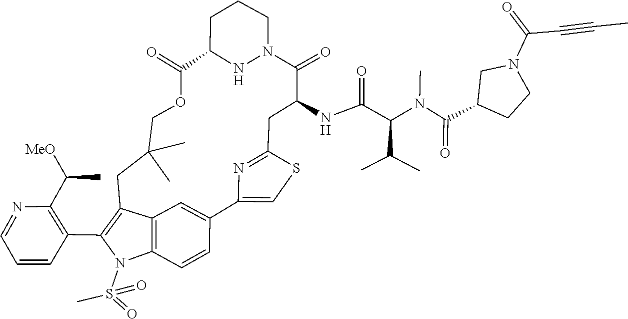 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4S(C)(=O)=O)CC(C)(C)COC(=O)[C@@H]3CCCN(N3)C2=O)C(C)C)C1] |
| BB124 | 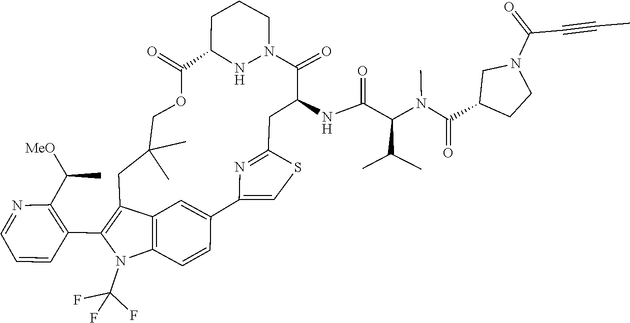 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(F)(F)F)CC(C)(C)COC(=O)[C@@H]3CCCN(N3)C2=O)C(C)C)C1] |
| BB126 | 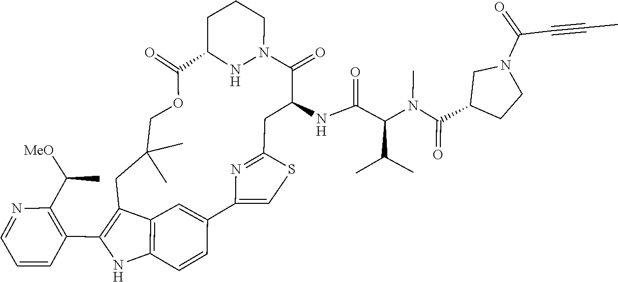 [SMILES: CC#CC(=O)N1CC[C@H](C(=O)N(C)[C@H](C(=O)N[C@H]2Cc3nc(cs3)-c3ccc4[nH]c(-c5cccnc5[C@H](C)OC)c(c4c3)CC(C)(C)COC(=O)[C@@H]3CCCN(N3)C2=O)C(C)C)C1] |
| BB127 | 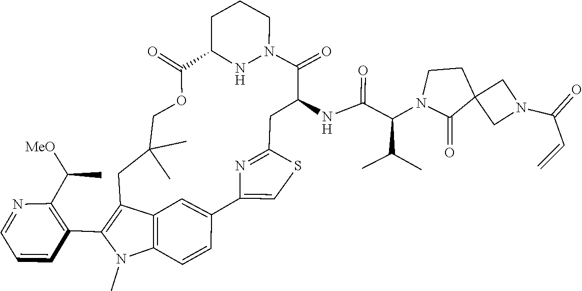 |
| BB128 | 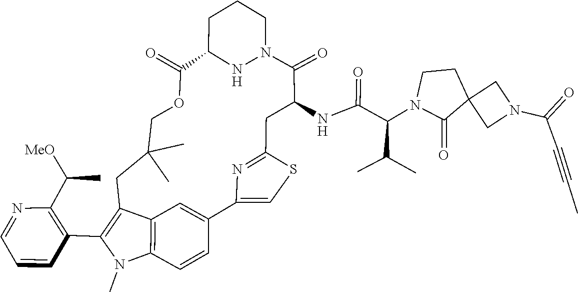 |
| BB129 | 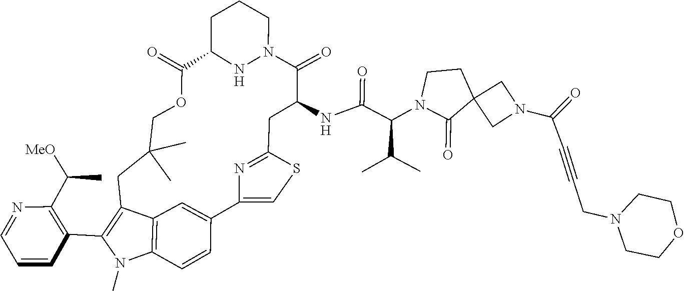 |
| BB130 | 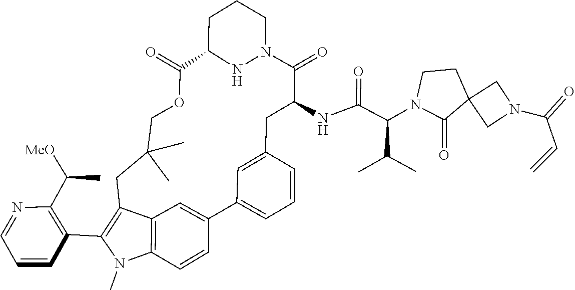 |
| BB131 | 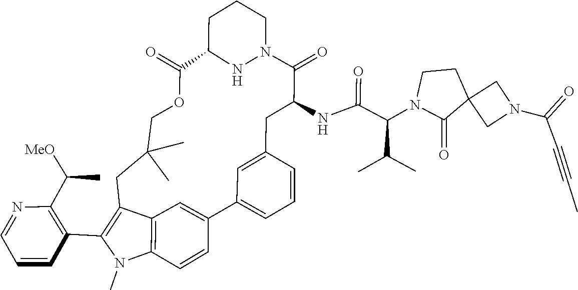 |
| BB132 | 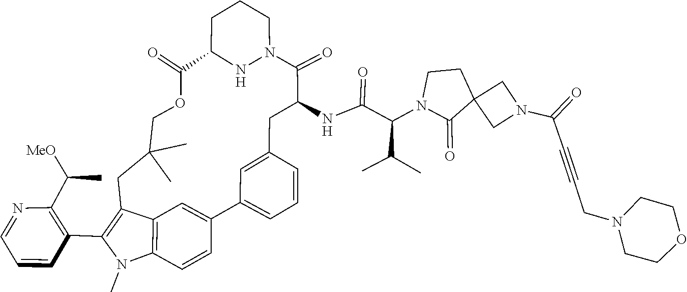 |
| BB139 | 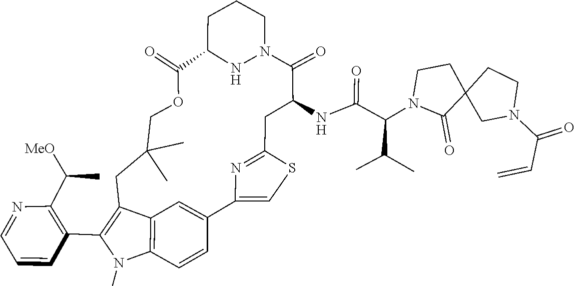 |
| BB140 | 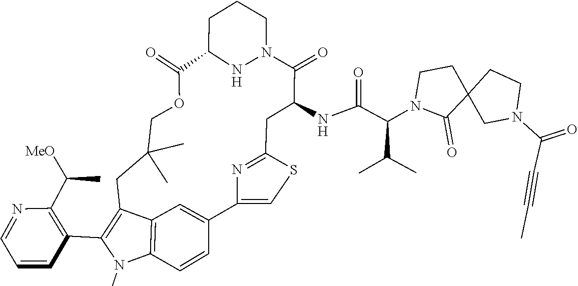 |
| BB141 | 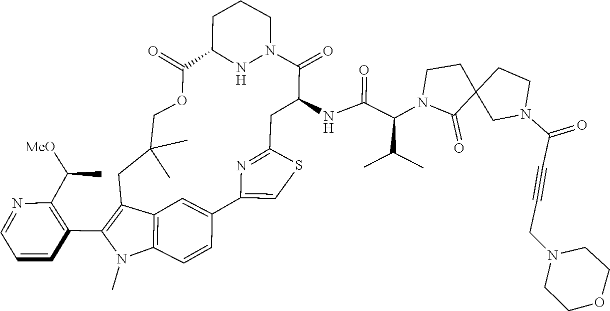 |
| BB142 | 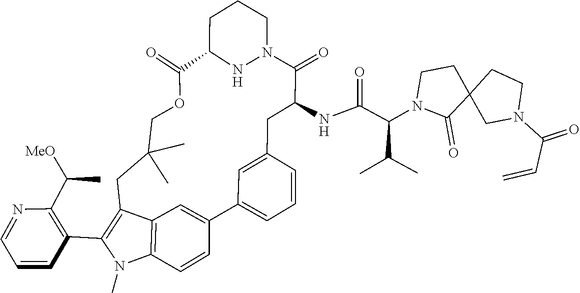 |
| BB143 | 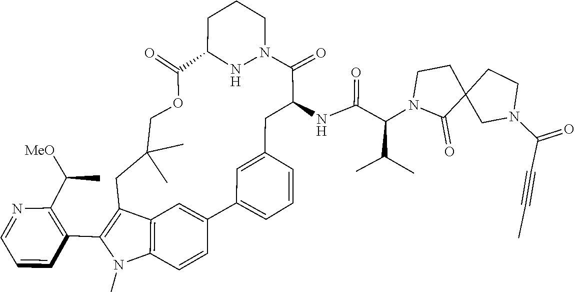 |
| BB144 | 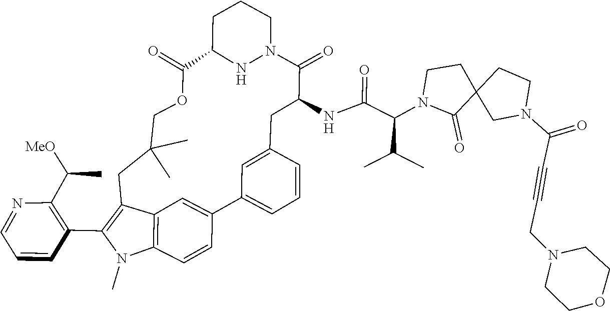 |
| BB145 | 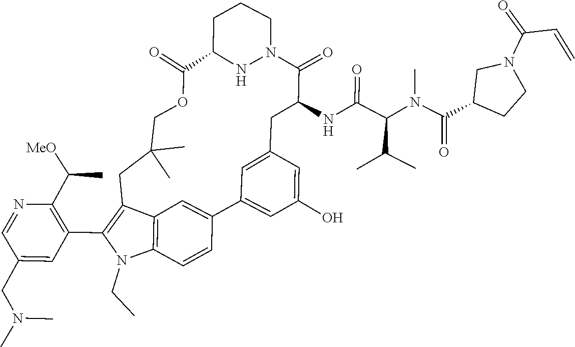 [SMILES: C=CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB146 | 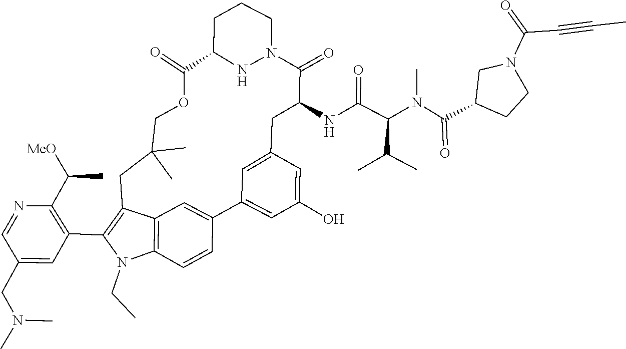 [SMILES: CC#CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB147 | 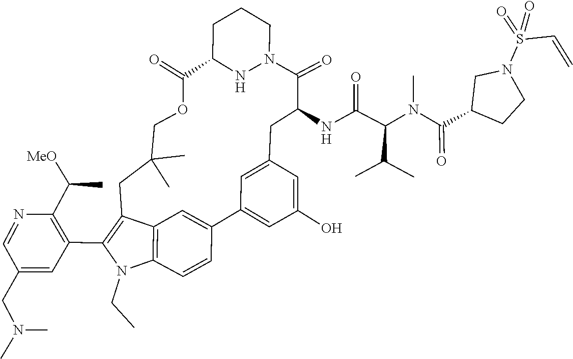 [SMILES: C=CS(=O)(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB148 | 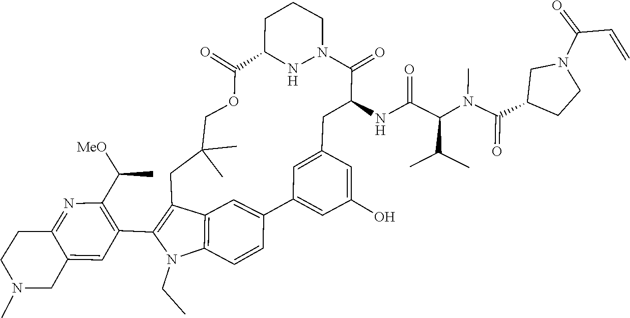 [SMILES: C=CC(=O)N1CC[C@H](C(=O)N(C)[C@H](C(=O)N[C@H]2Cc3cc(O)cc(c3)-c3ccc4c(c3)c(c(-c3cc5c(nc3[C@H](C)OC)CCN(C)C5)n4CC)CC(C)(C)COC(=O)[C@@H]3CCCN(N3)C2=O)C(C)C)C1] |
| BB149 | 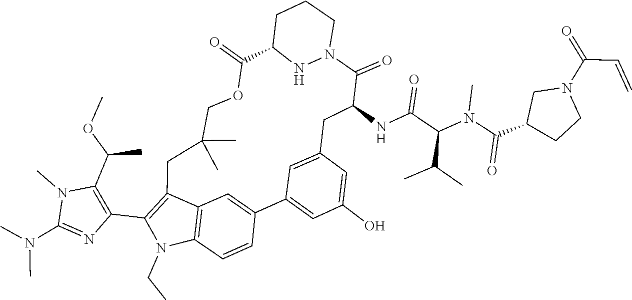 [SMILES: C=CC(=O)N1CC[C@H](C(=O)N(C)[C@H](C(=O)N[C@H]2Cc3cc(O)cc(c3)-c3ccc4c(c3)c(c(-c3nc(N(C)C)n(C)c3[C@H](C)OC)n4CC)CC(C)(C)COC(=O)[C@@H]3CCCN(N3)C2=O)C(C)C)C1] |
| BB150 | 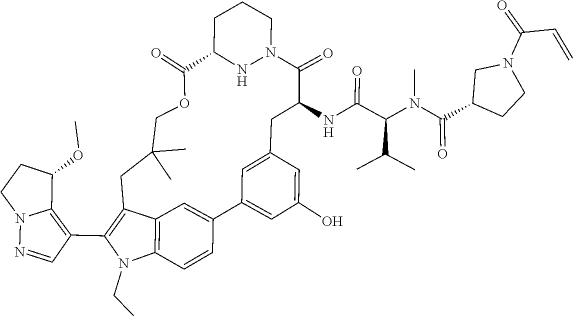 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CC5)n4CC)CC(C)(C)COC(=O)[C@@H]3CCCN(N3)C2=O)C(C)C)C1] |
| BB161 | 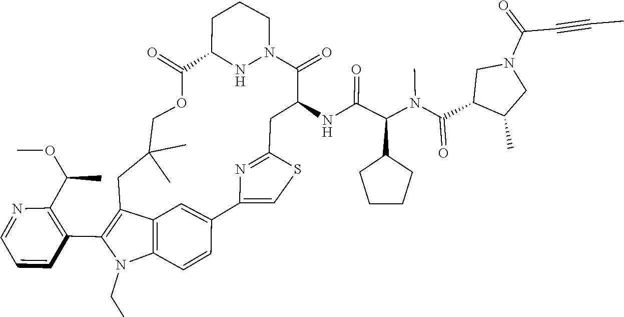 |
| BB162 | 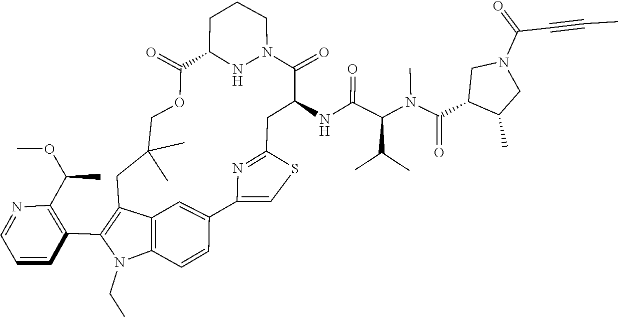 |
| BB163 | 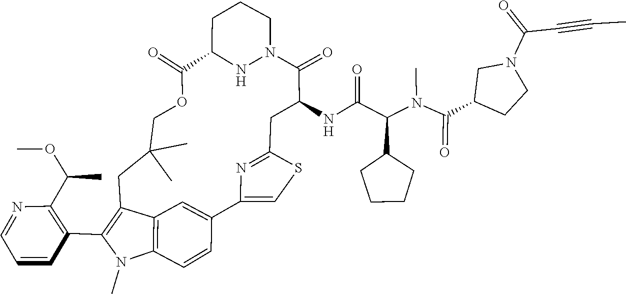 |
| BB164 | 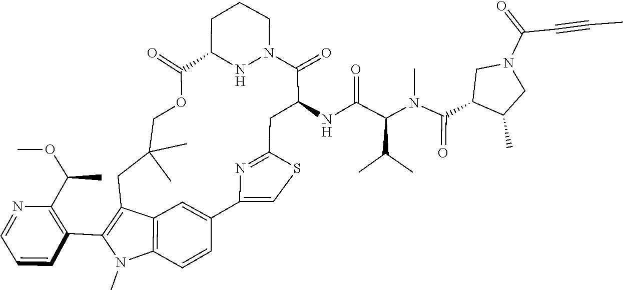 |
| BB165 | 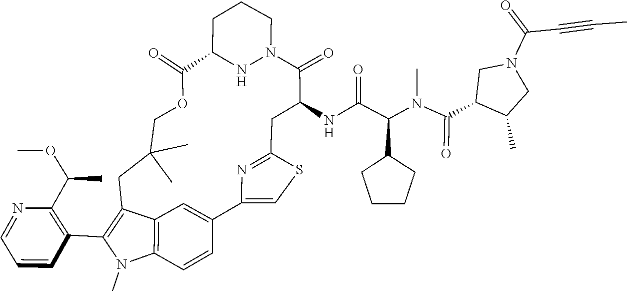 |
| BB167 | 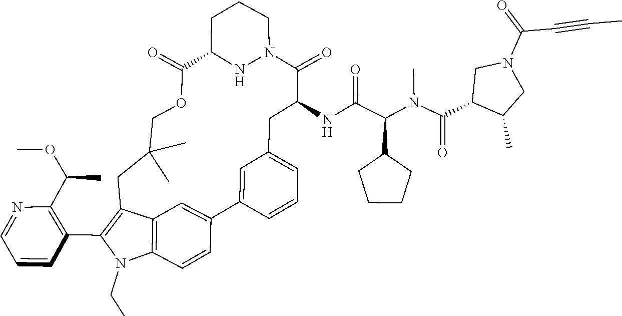 |
| BB168 | 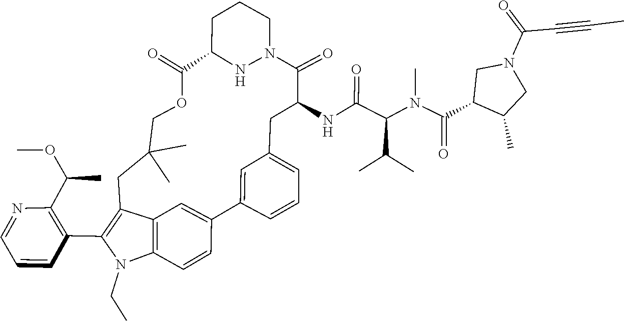 |
| BB169 | 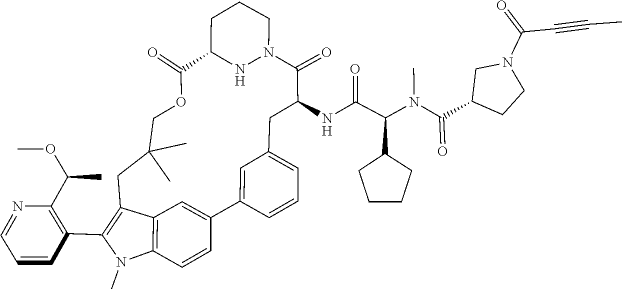 |
| BB170 | 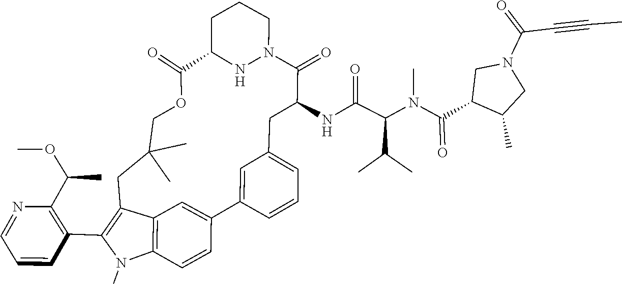 |
| BB171 | 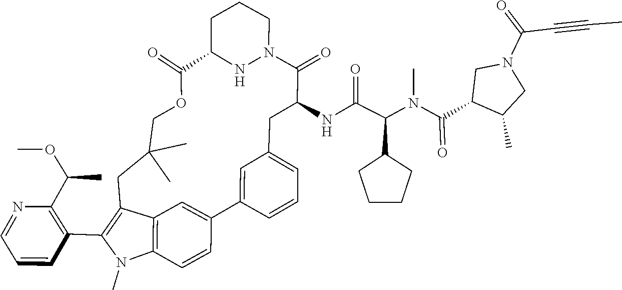 |
| BB172 | 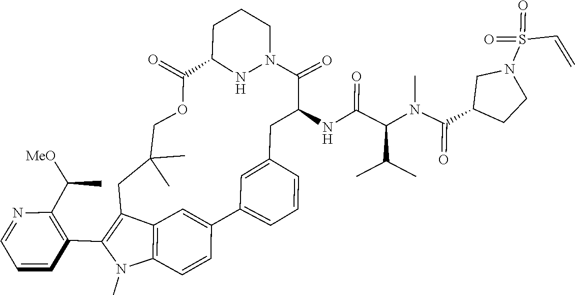 |
| BB173 | 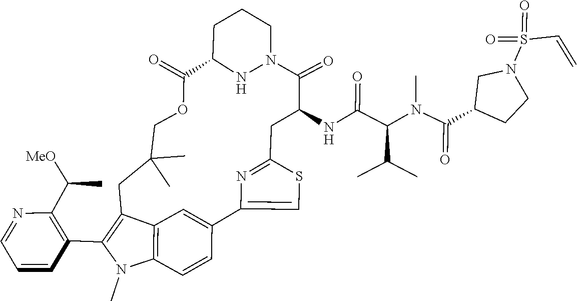 |
| BB174 | 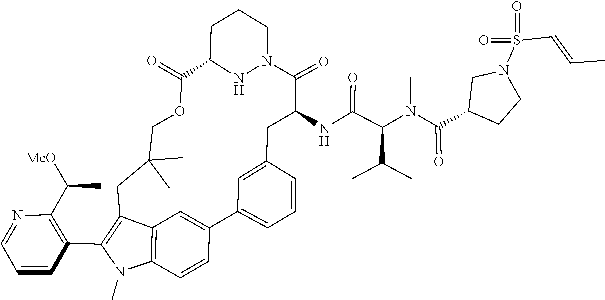 |
| BB175 | 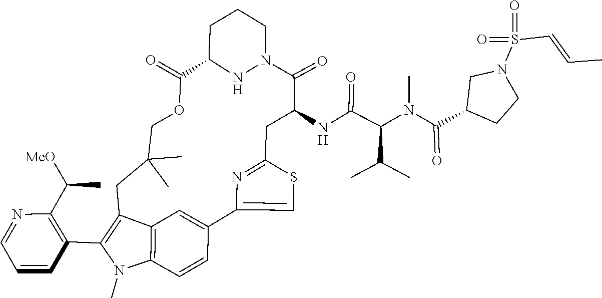 |
| BB176 | 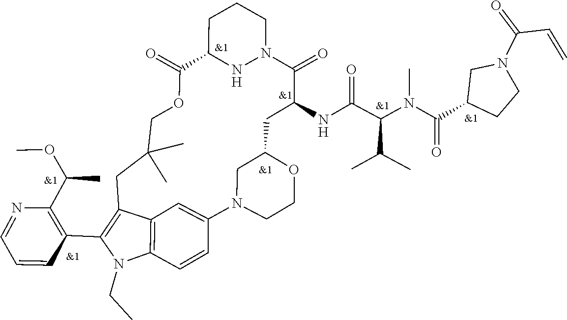 |
| BB177 | 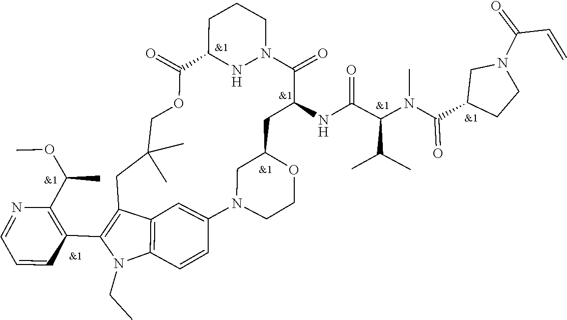 |
| BB178 | 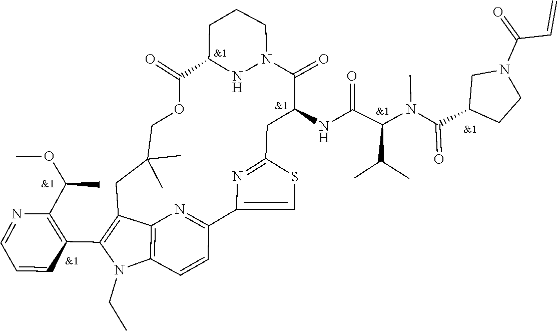 |
| BB179 | 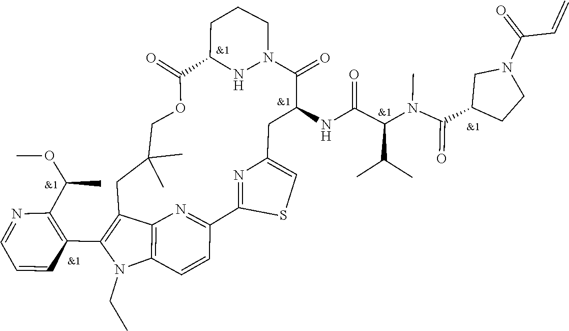 |
| BB180 | 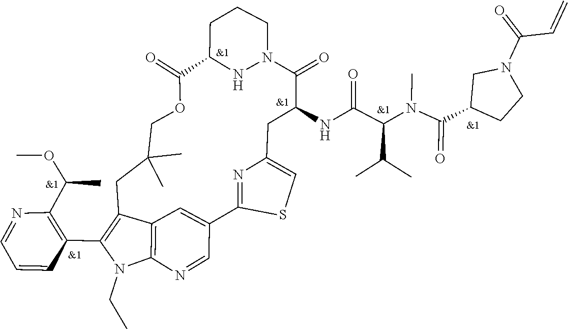 |
| BB181 | 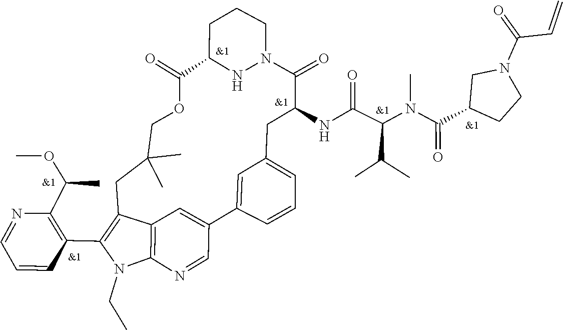 |
| BB182 | 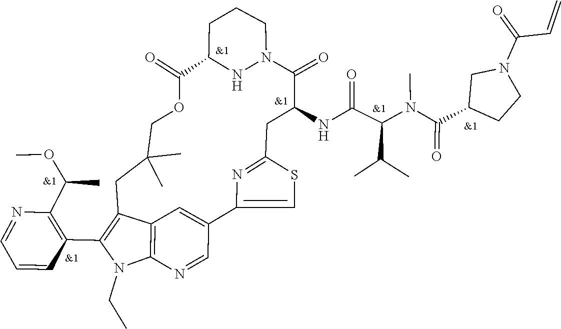 |
| BB183 | 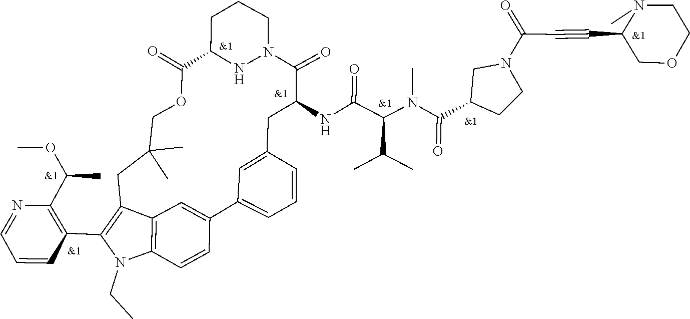 |
| BB184 | 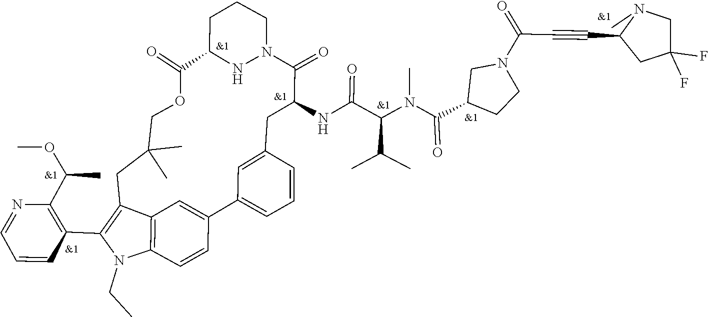 |
| BB185 | 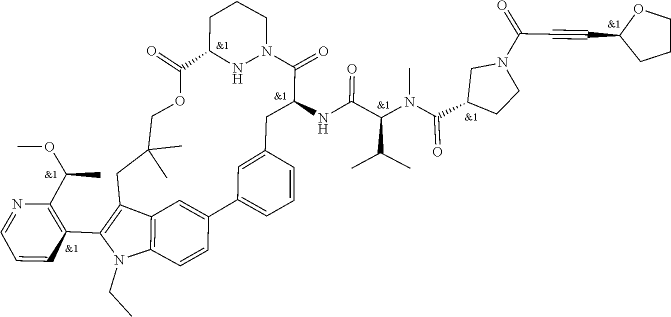 |
| BB186 | 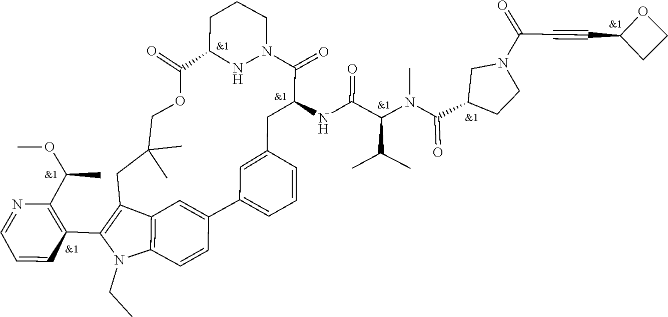 |
| BB187 | 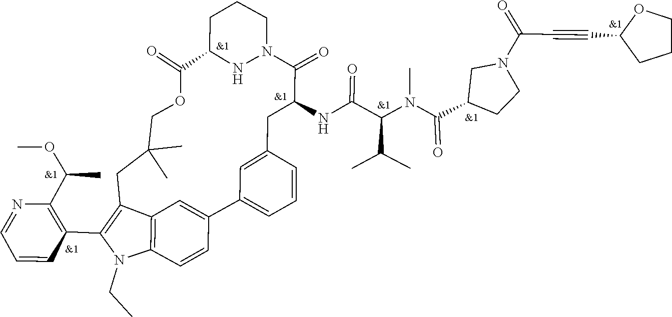 |
| BB188 | 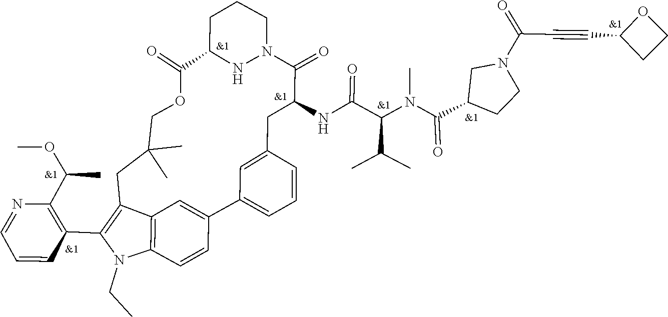 |
| BB189 | 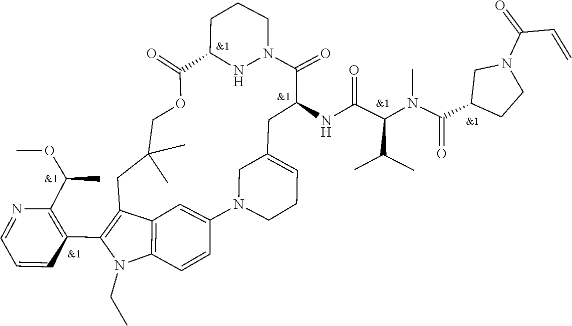 |
| BB190 | 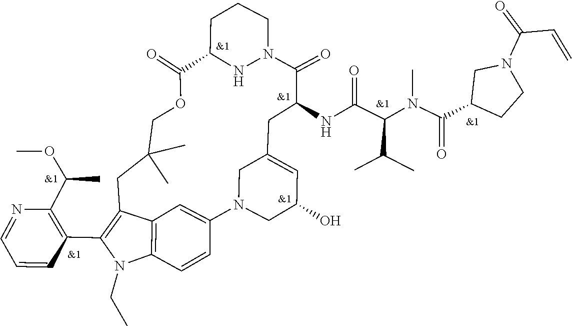 |
| BB191 | 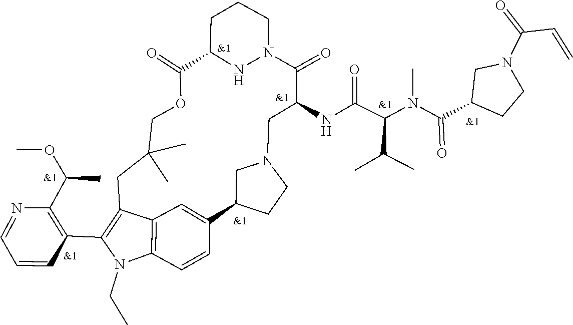 |
| BB192 | 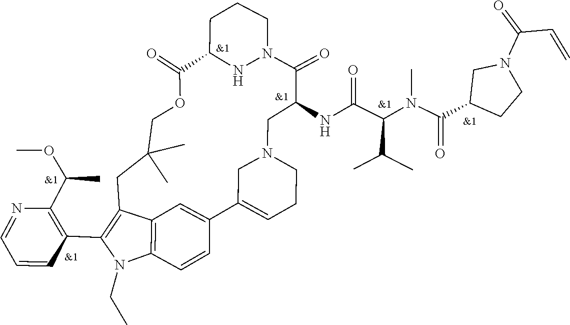 |
| BB194 | 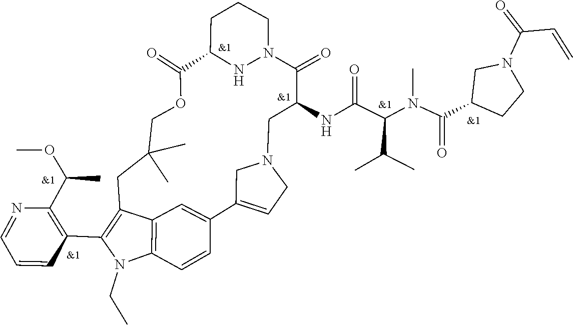 |
| BB195 | 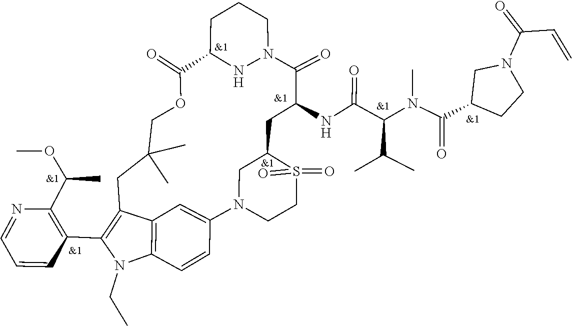 |
| BB196 | 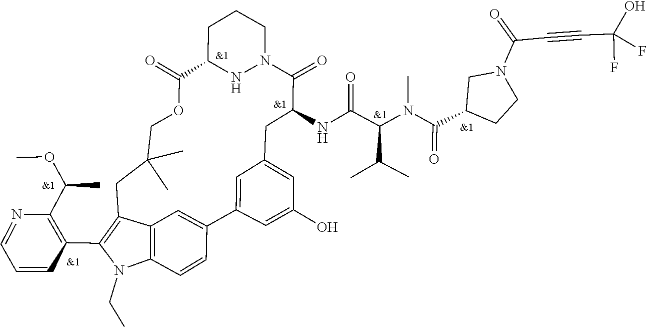 |
| BB197 | 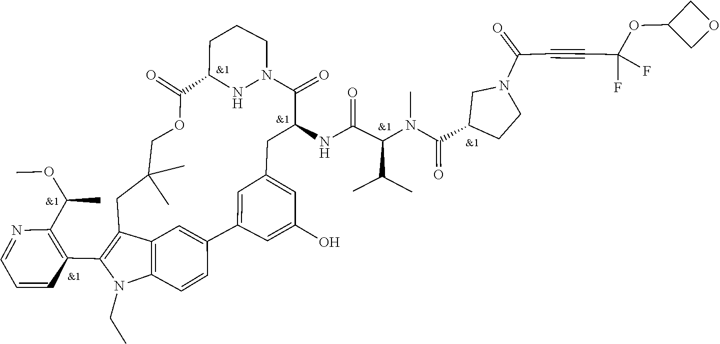 |
| BB198 | 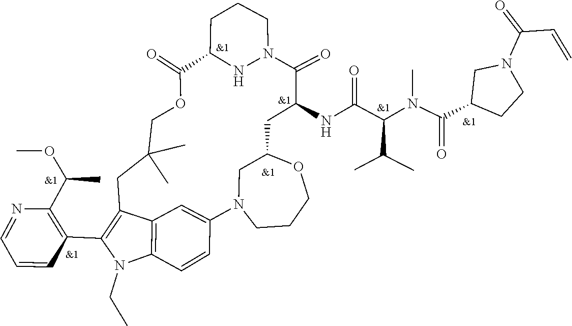 |
| BB199 | 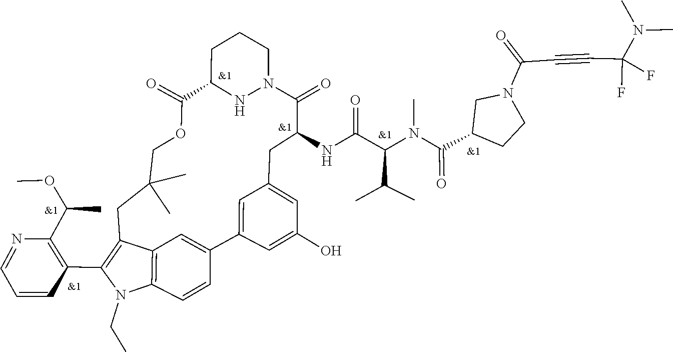 |
| BB200 | 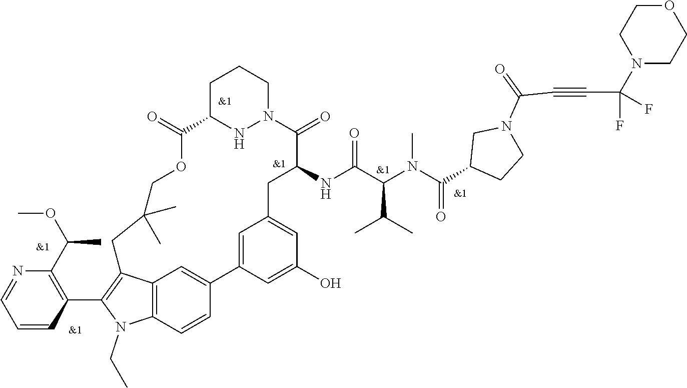 |
| BB201 | 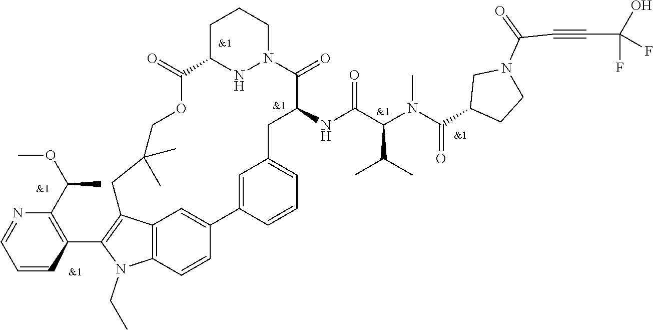 |
| BB202 | 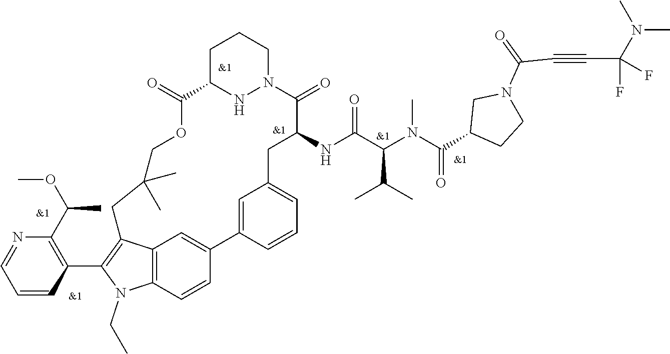 |
| BB203 | 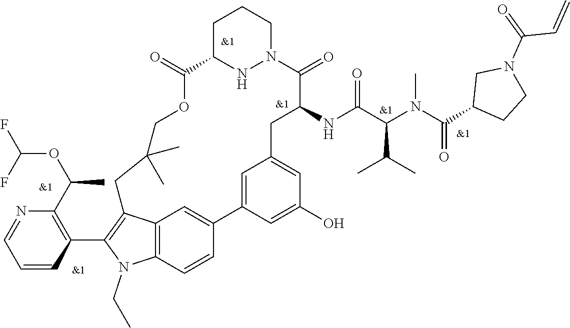 |
| BB204 | 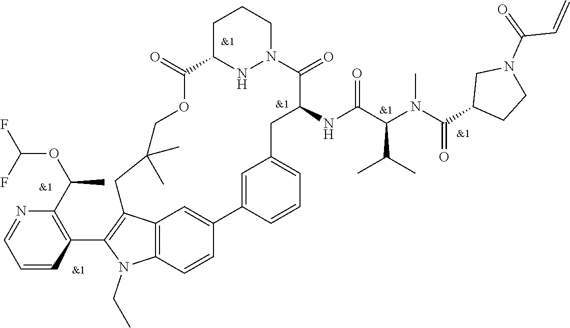 |
| BB205 | 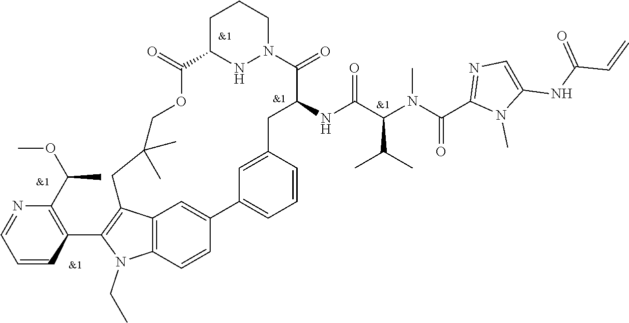 |
| BB206 | 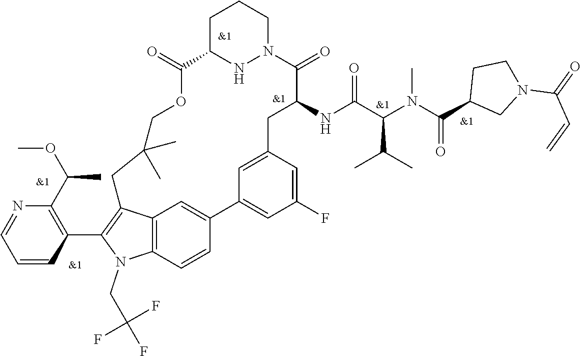 |
| BB207 | 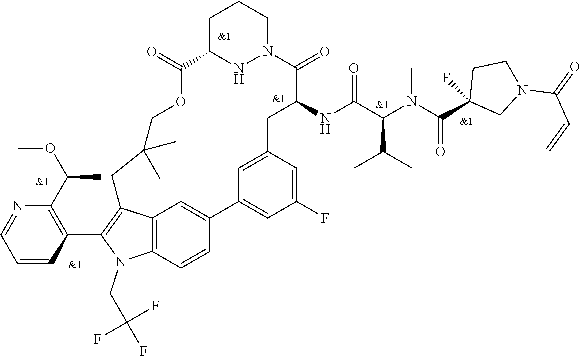 |
| BB208 | 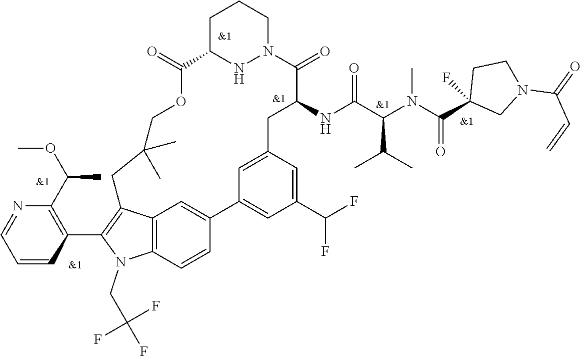 |
| BB209 | 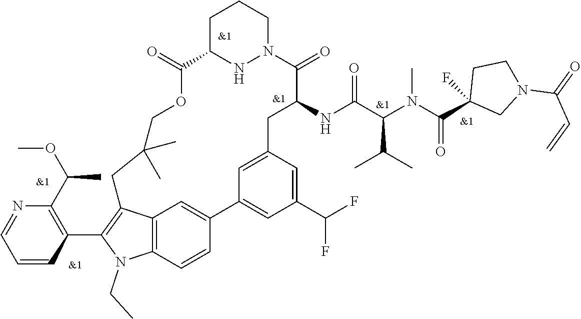 |
| BB210 | 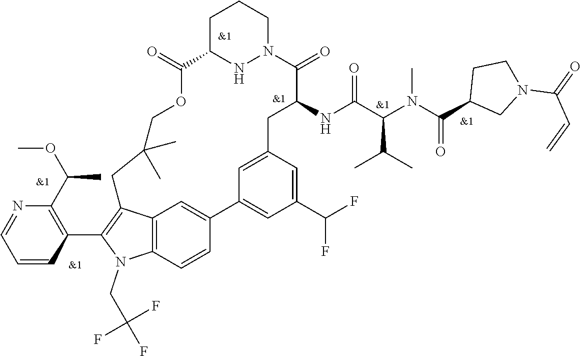 |
| BB211 | 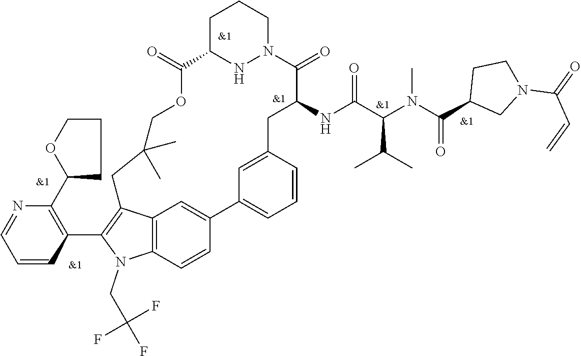 |
| BB212 | 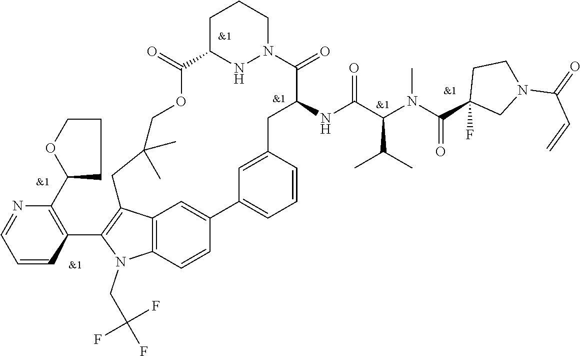 |
| BB213 | 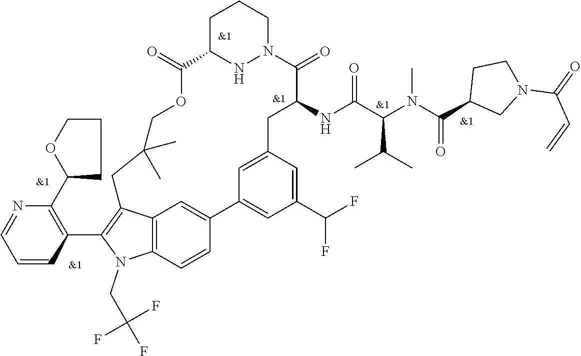 |
| BB214 | 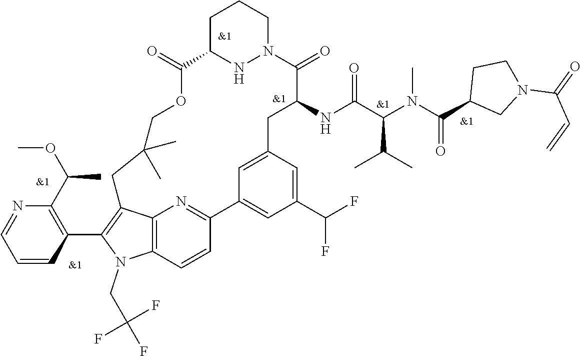 |
| BB215 | 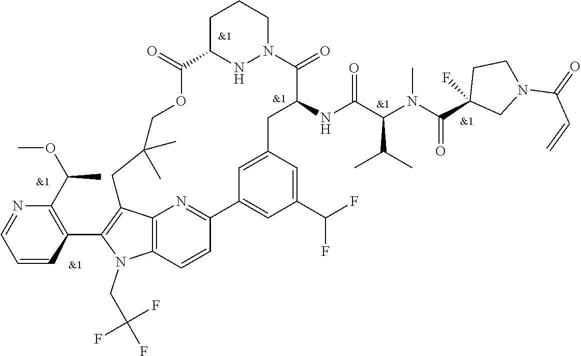 |
| BB216 | 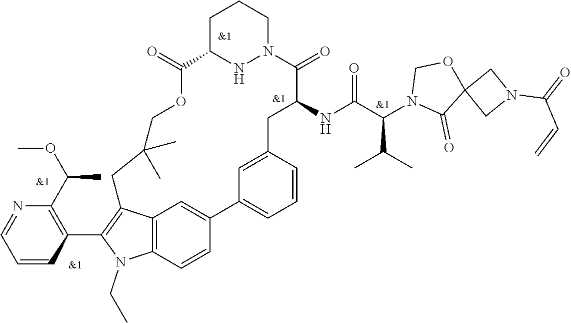 |
| BB217 | 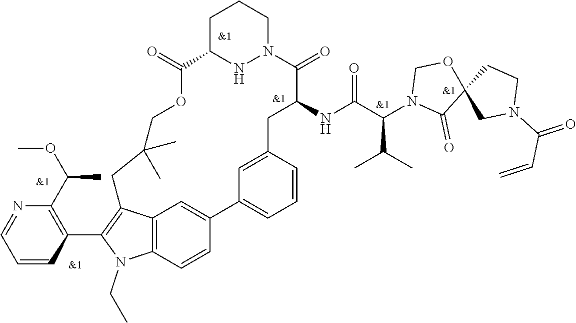 |
| BB218 | 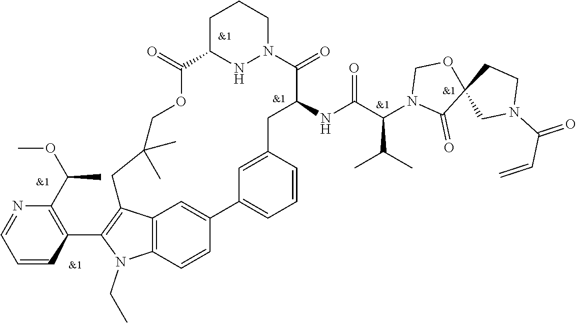 |
| BB219 | 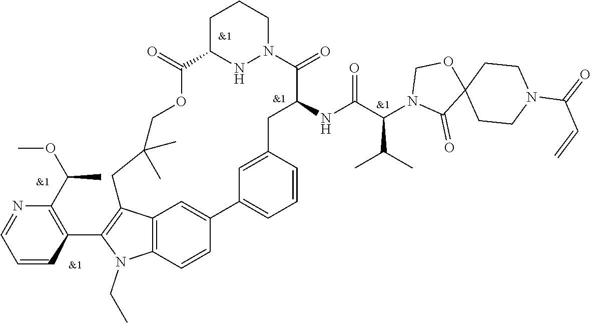 |
| BB220 | 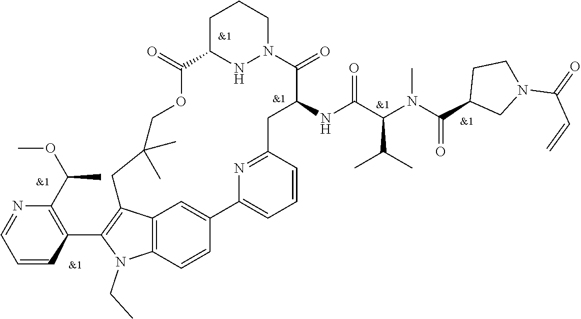 |
| BB221 | 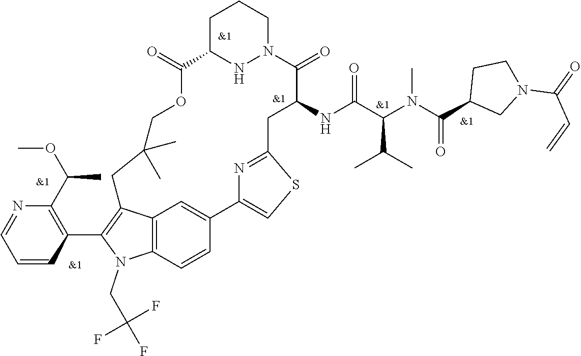 |
| BB222 | 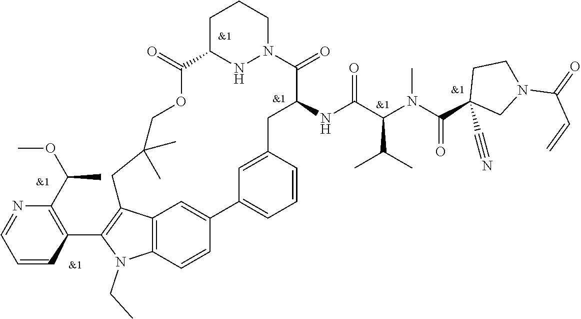 |
| BB223 | 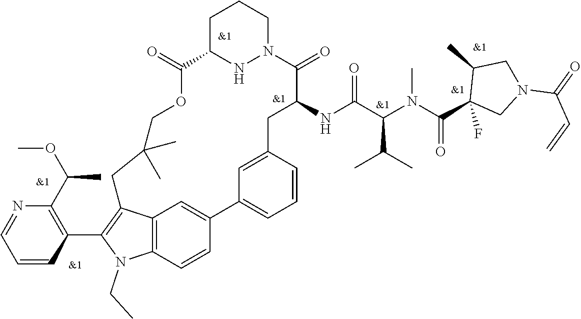 |
| BB224 | 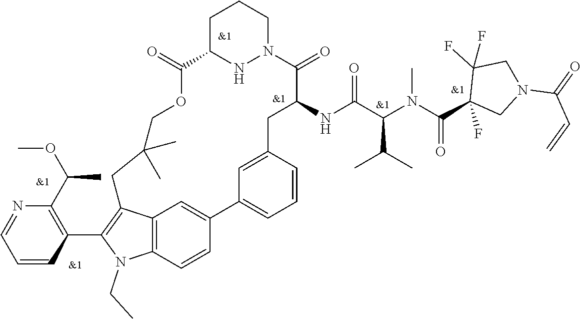 |
| BB225 | 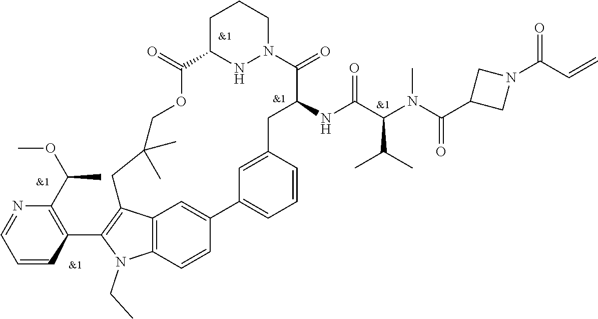 |
| BB226 | 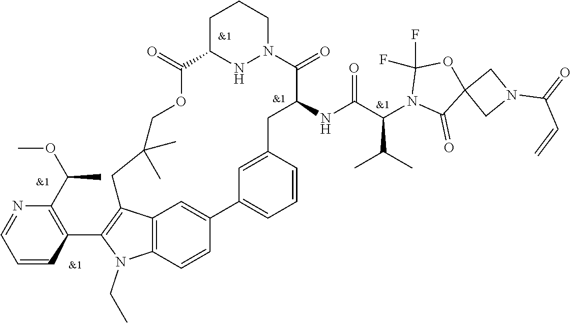 |
| BB227 | 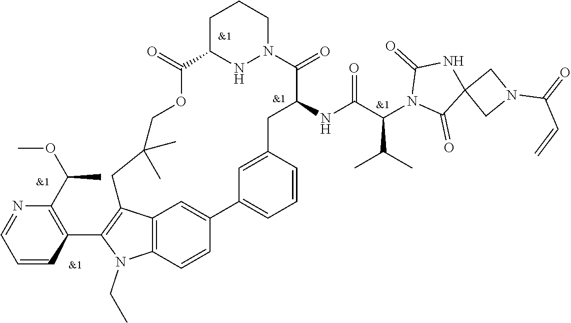 |
| BB228 | 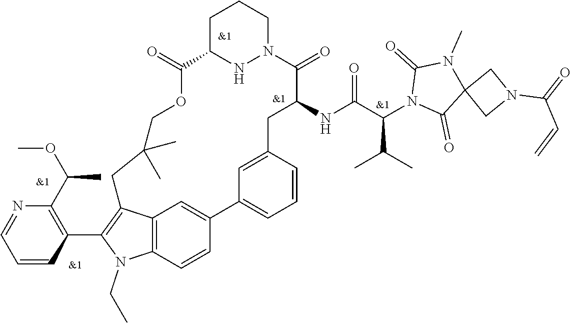 |
| BB229 | 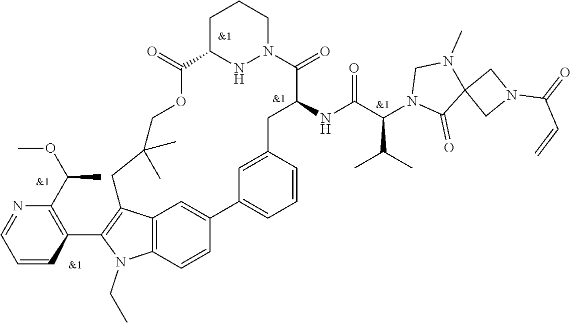 |
| BB230 | 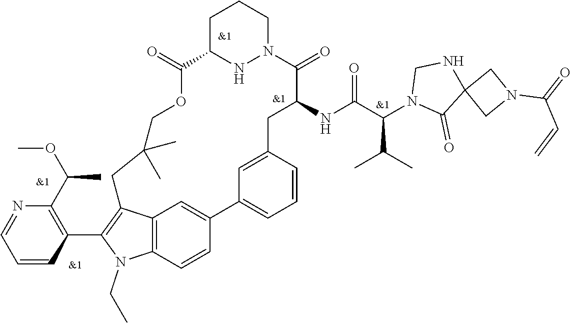 |
| BB231 | 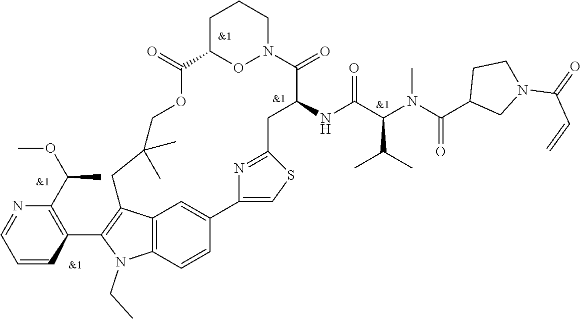 |
| BB232 | 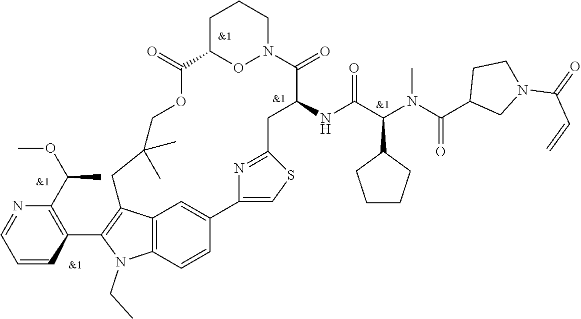 |
| BB233 | 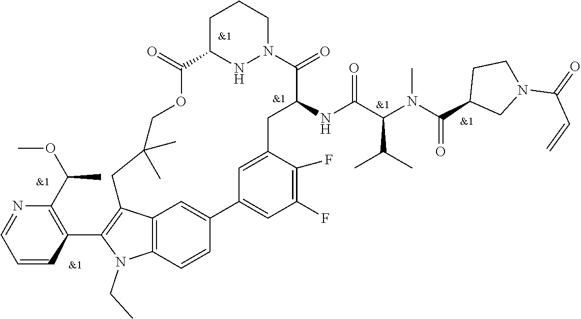 |
| BB234 | 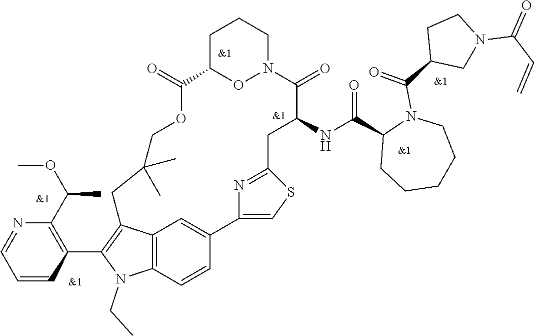 |
| BB235 | 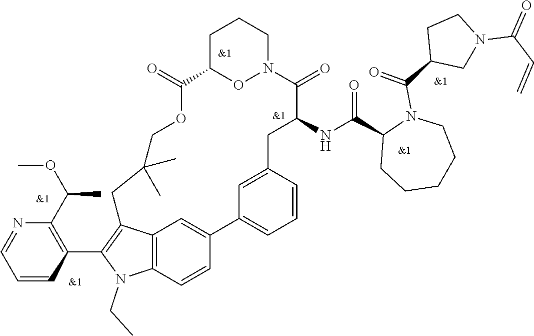 |
| BB236 | 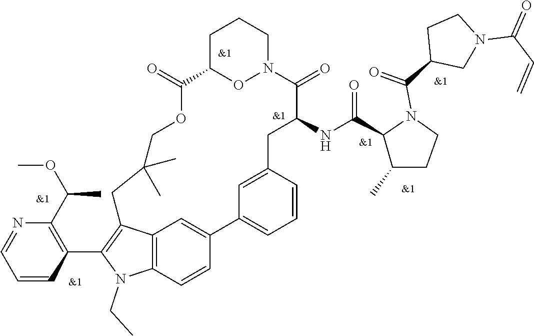 |
| BB237 | 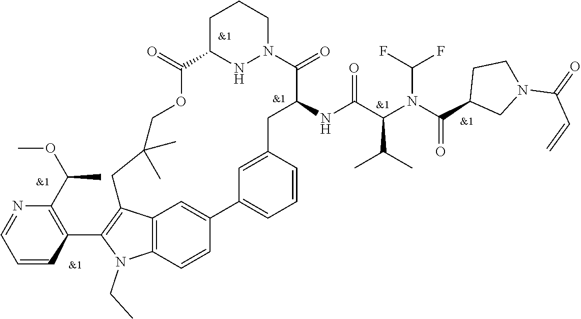 |
| BB238 | 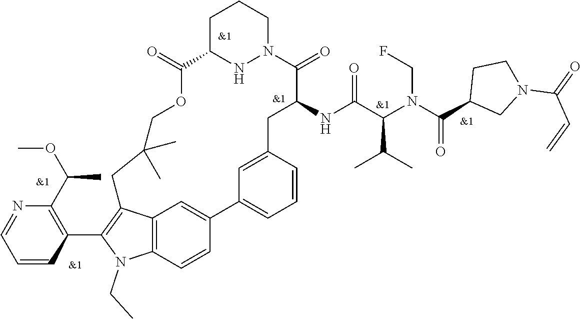 |
| BB239 | 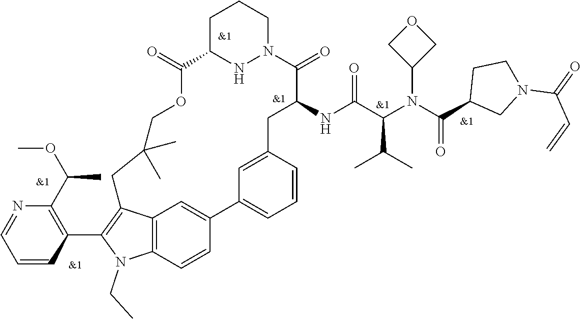 |
| BB240 | 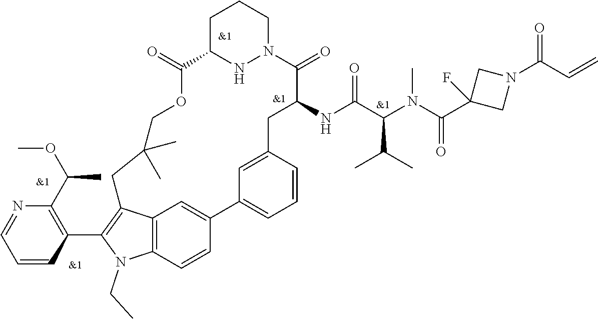 |
| BB241 | 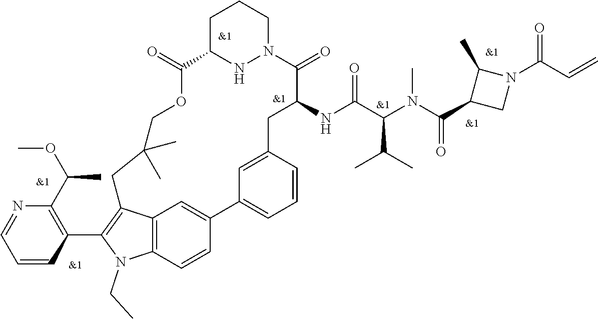 |
| BB242 | 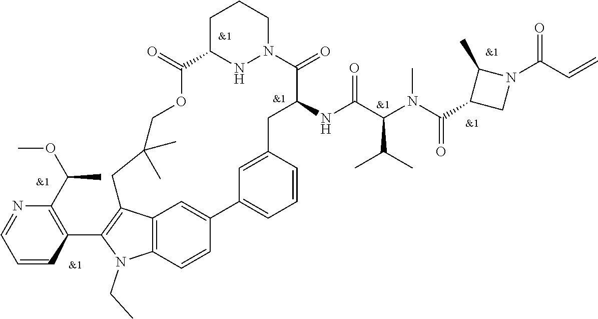 |
| BB243 | 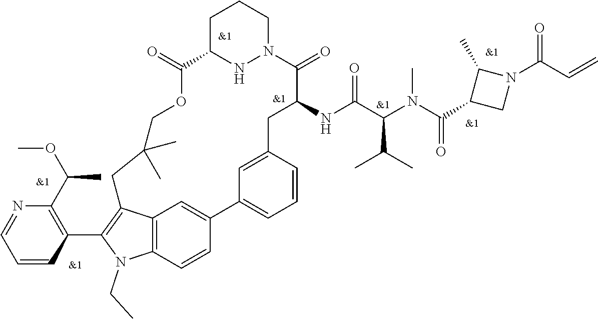 |
| BB244 | 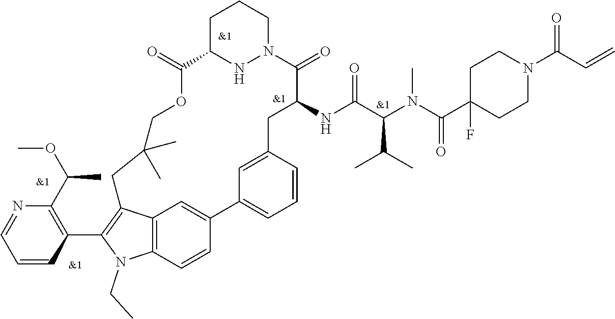 |
| BB245 | 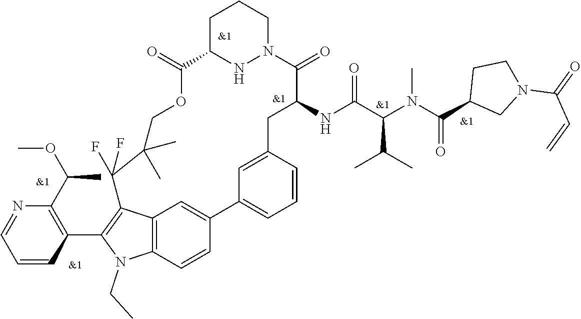 |
| BB246 | 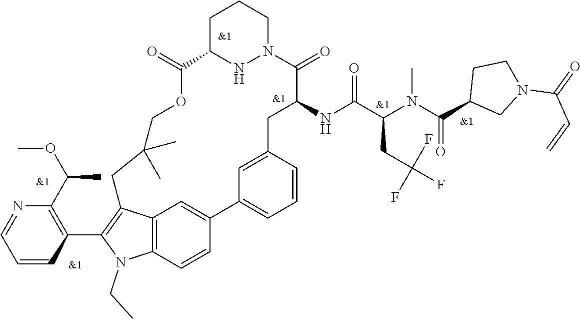 |
| BB247 | 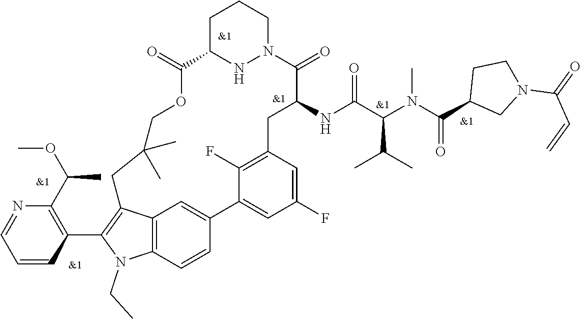 |
| BB248 | 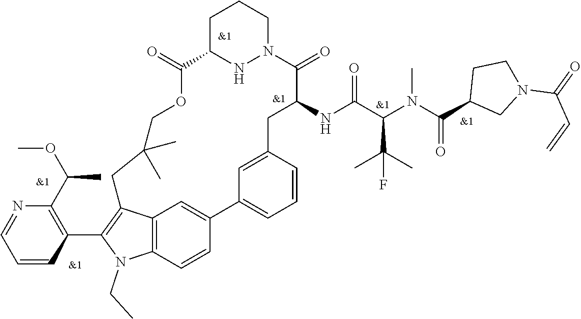 |
| BB249 | 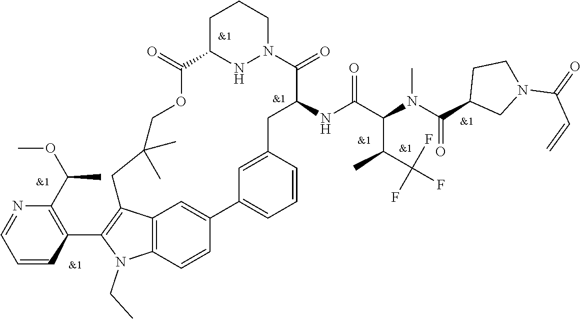 |
| BB250 | 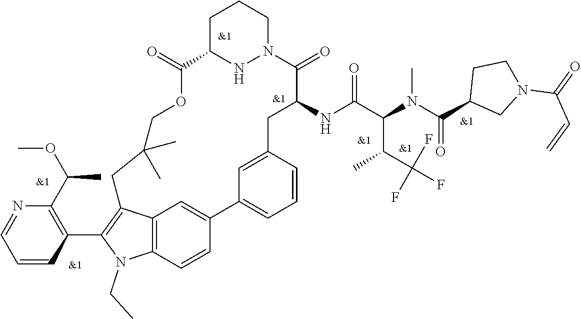 |
| BB251 | 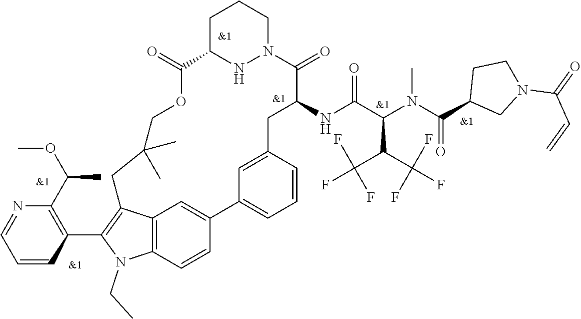 |
| BB252 | 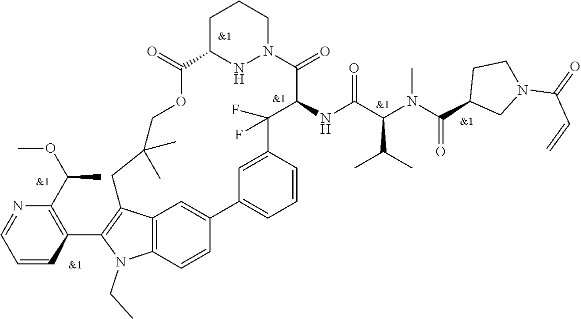 |
| BB253 | 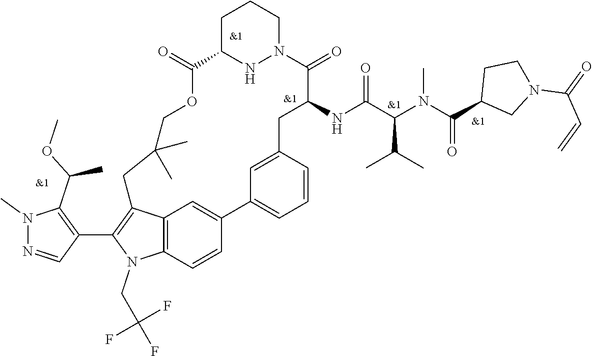 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)COC(=O)[C@@H]3CCCN(N3)C2=O)C(C)C)C1] |
| BB254 | 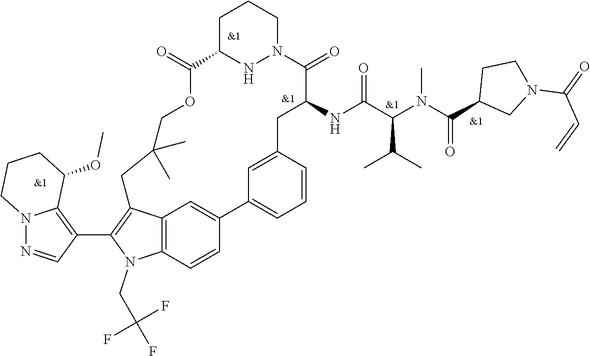 [SMILES: C=CC(=O)N1CC[C@H](C(=O)N(C)[C@H](C(=O)N[C@H]2Cc3cccc(c3)-c3ccc4c(c3)c(c(-c3cnn5c3[C@@H](OC)CCC5)n4CC(F)(F)F)CC(C)(C)COC(=O)[C@@H]3CCCN(N3)C2=O)C(C)C)C1] |
| BB255 | 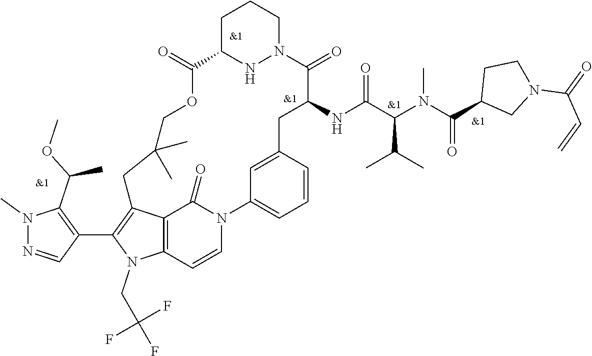 [SMILES: C=CC(=O)N1CC[C@H](C(=O)N(C)[C@H](C(=O)N[C@H]2Cc3cccc(c3)-n3ccc4c(c(c(-c5cnn(C)c5[C@H](C)OC)n4CC(F)(F)F)CC(C)(C)COC(=O)[C@@H]4CCCN(N4)C2=O)c3=O)C(C)C)C1] |
| BB256 | 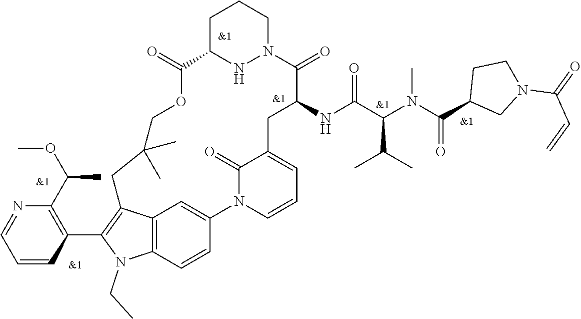 |
| BB257 | 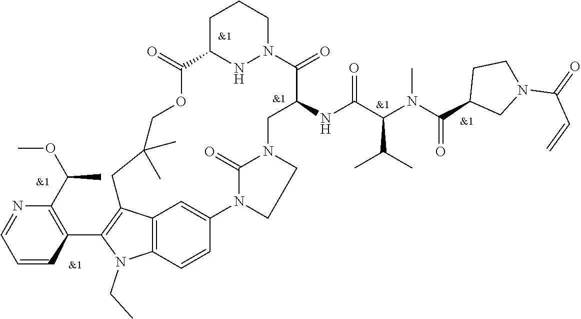 |
| BB258 | 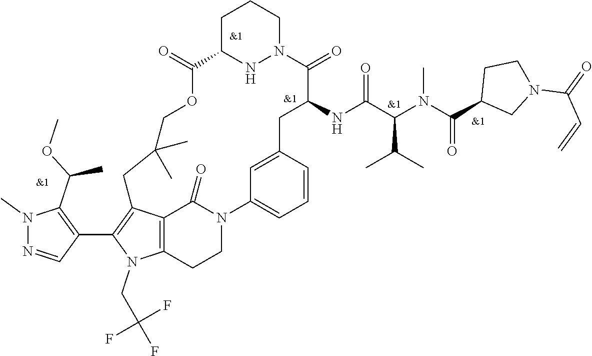 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3=O)C(C)C)C1] |
| BB259 | 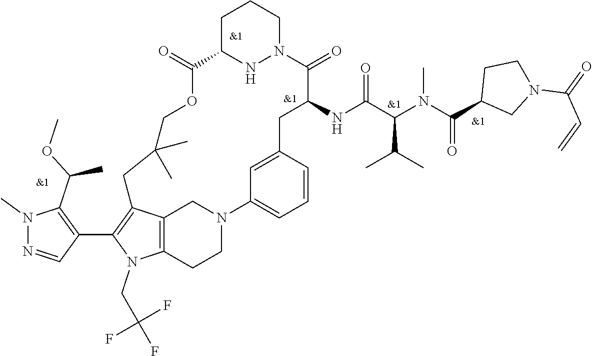 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| BB260 | 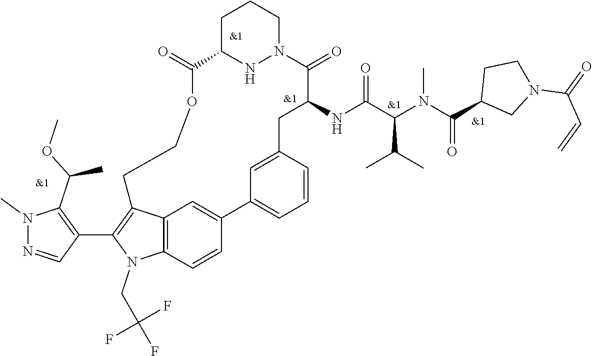 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CCOC(=O)[C@@H]3CCCN(N3)C2=O)C(C)C)C1] |
| BB261 | 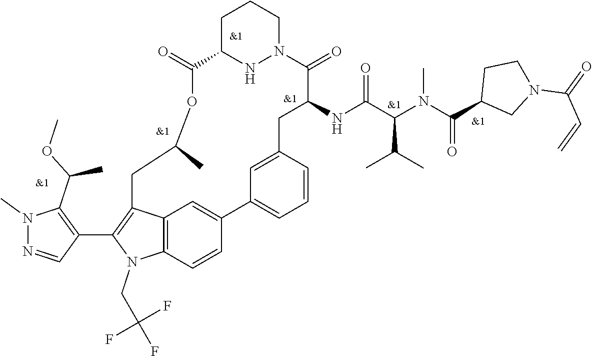 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@H](C)OC(=O)[C@@H]3CCCN(N3)C2=O)C(C)C)C1] |
| BB262 | 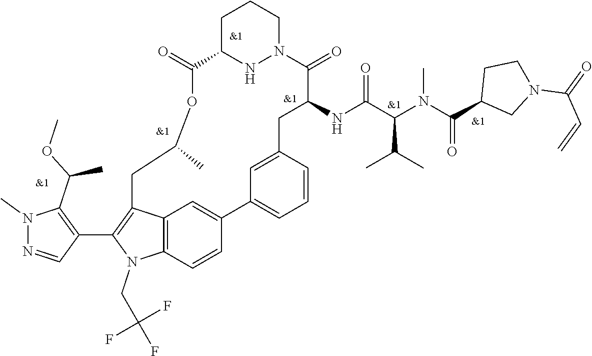 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@@H](C)OC(=O)[C@@H]3CCCN(N3)C2=O)C(C)C)C1] |
| BB263 | 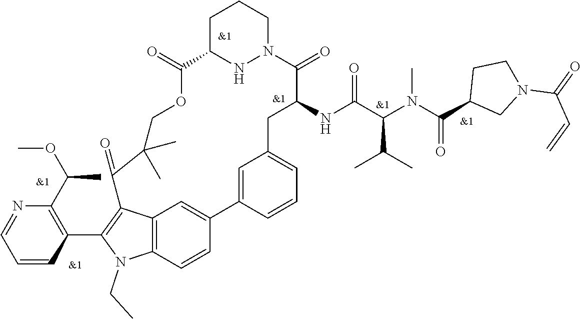 |
| BB264 | 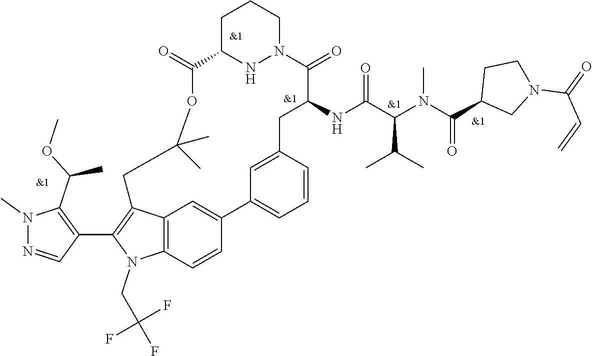 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)OC(=O)[C@@H]3CCCN(N3)C2=O)C(C)C)C1] |
| BB265 | 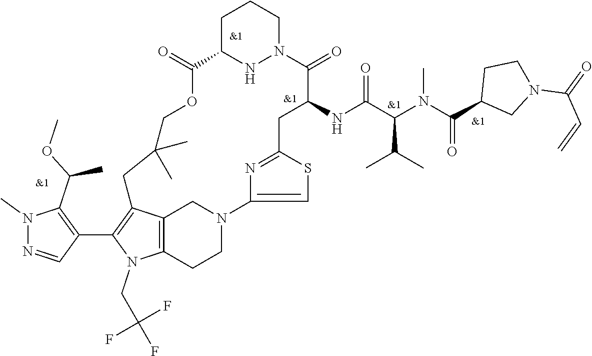 [SMILES: C=CC(=O)N1CC[C@H](C(=O)N(C)[C@H](C(=O)N[C@H]2Cc3nc(cs3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| BB266 | 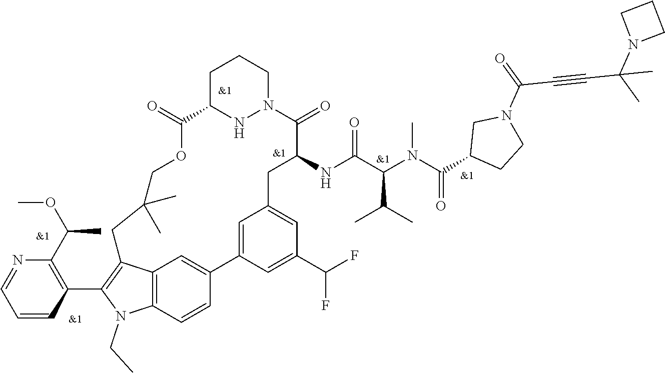 |
| BB267 | 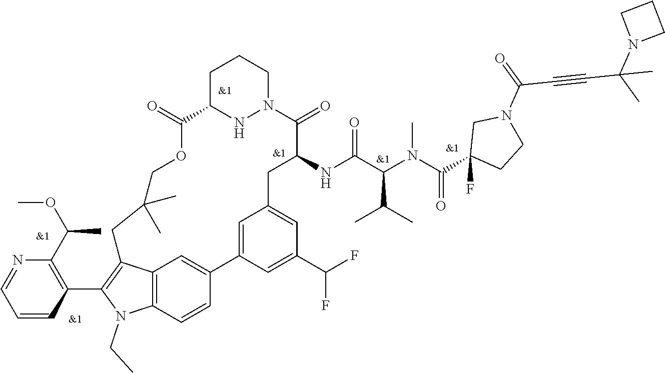 |
| BB268 | 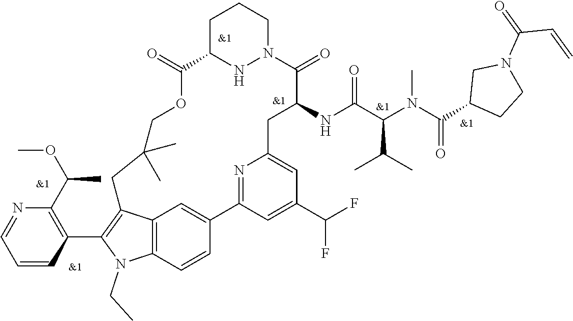 |
| BB269 | 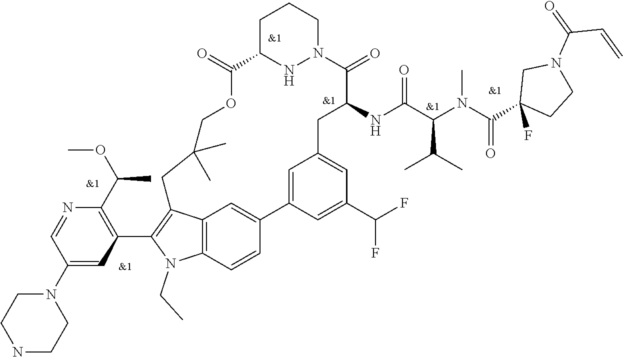 |
| BB270 | 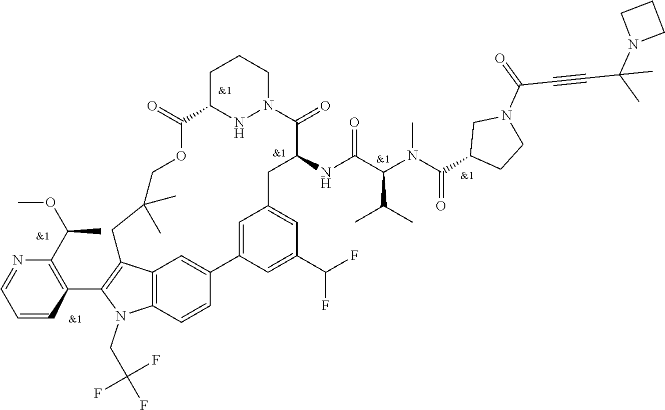 |
| BB271 | 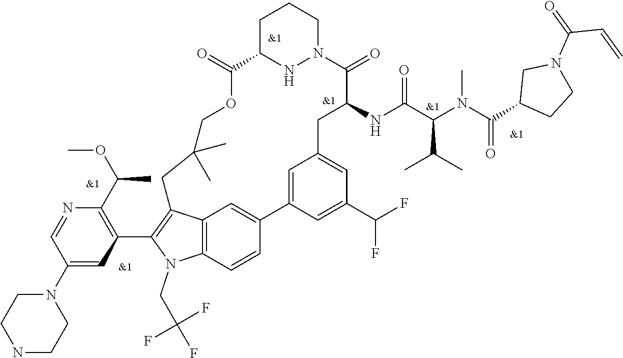 |
| BB272 | 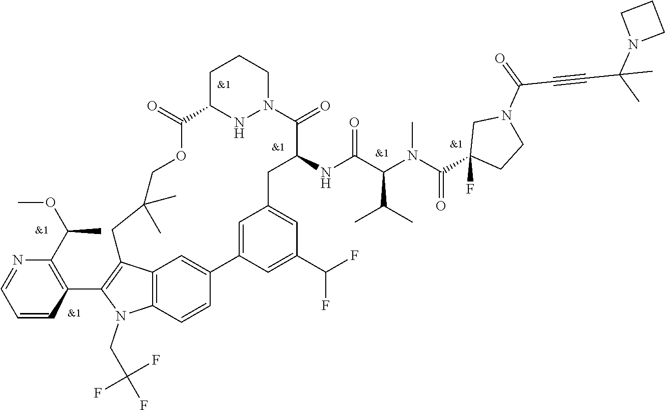 |
| BB273 | 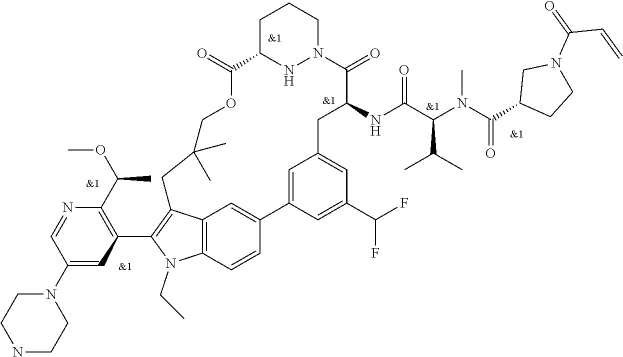 |
| BB274 | 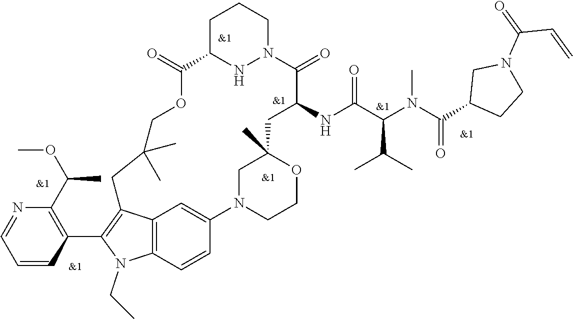 |
| BB275 | 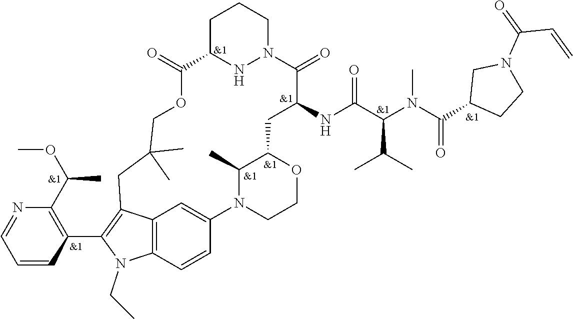 |
| BB276 | 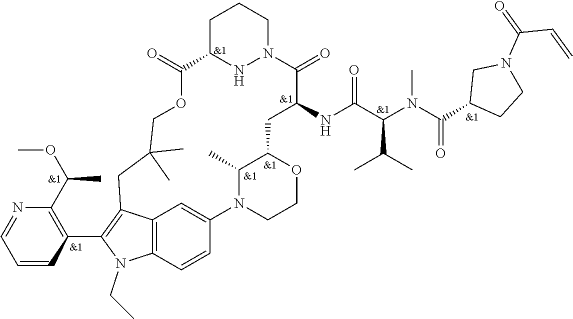 |
| BB277 | 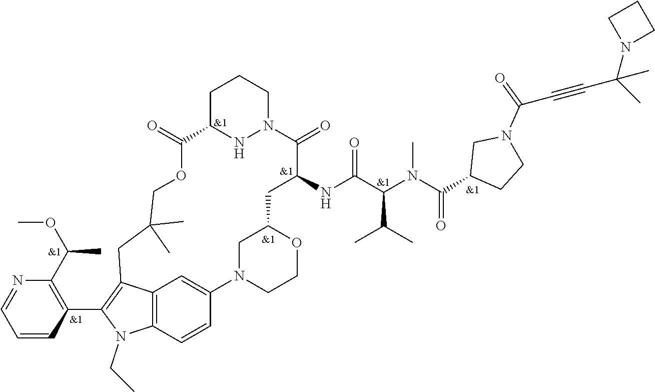 |
| BB278 | 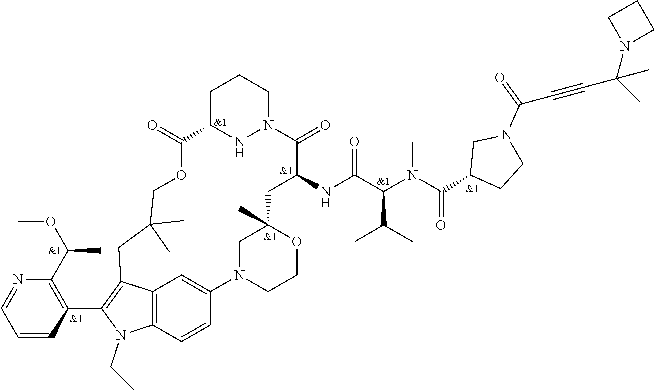 |
| BB279 | 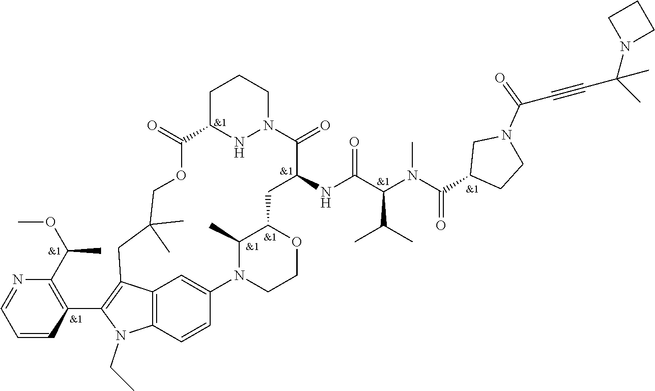 |
| BB280 | 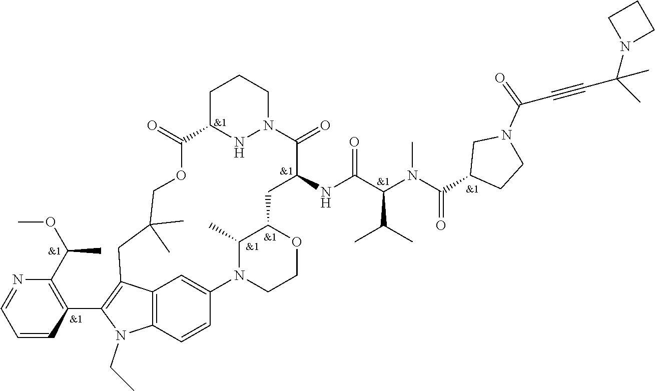 |
| BB282 | 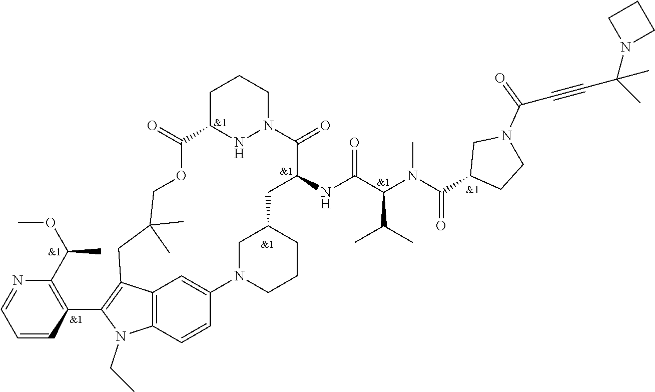 |
| BB283 | 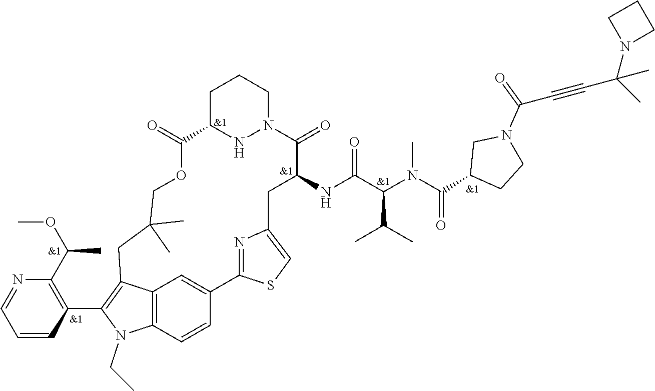 |
| BB284 | 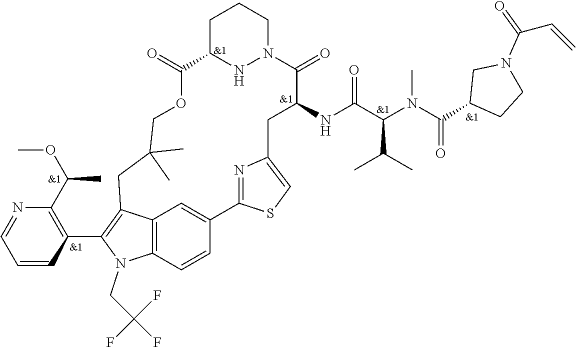 |
| BB285 | 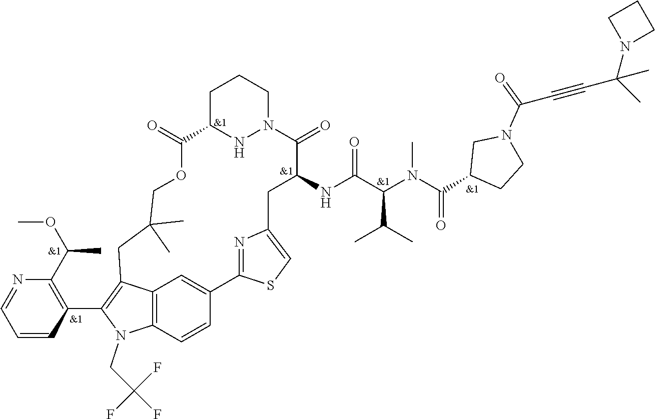 |
| BB286 | 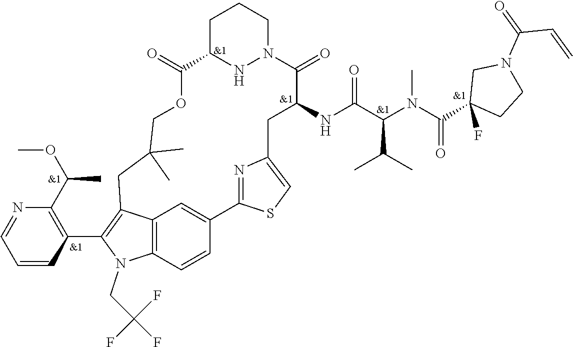 |
| BB287 | 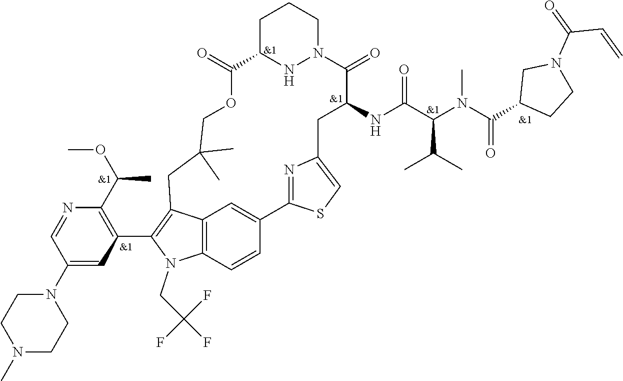 |
| BB288 | 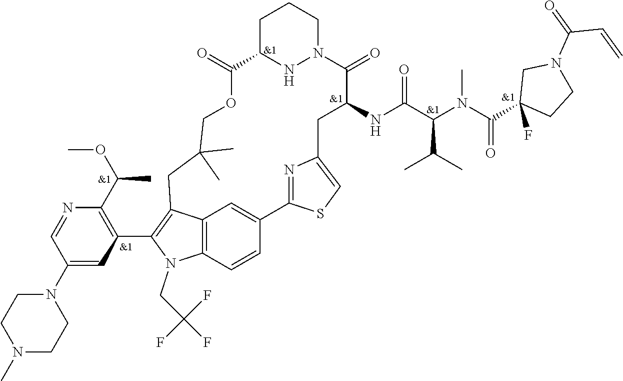 |
| BB289 | 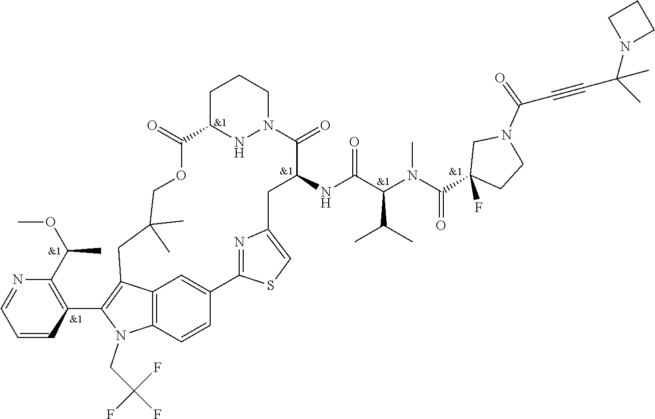 |
| BB290 | 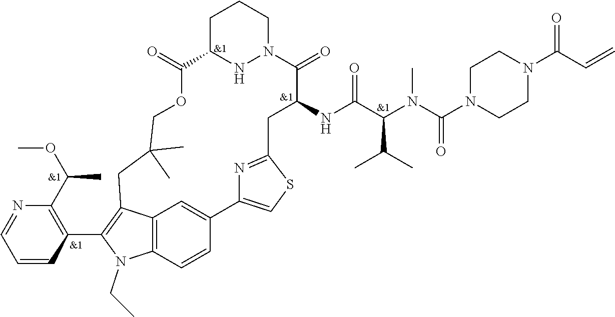 |
| BB291 | 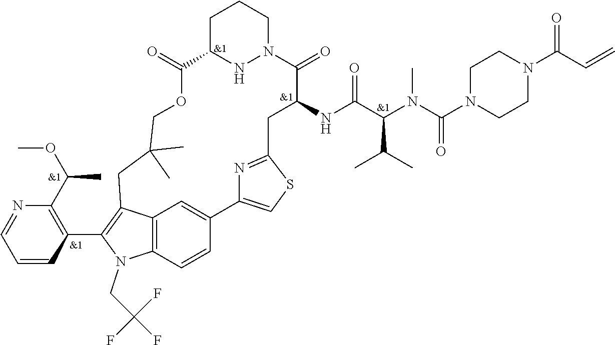 |
| BB292 | 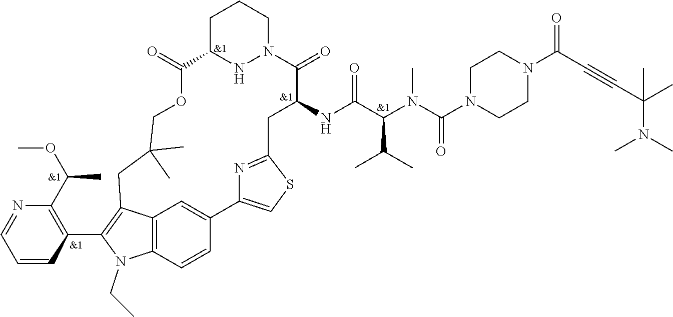 |
| BB293 | 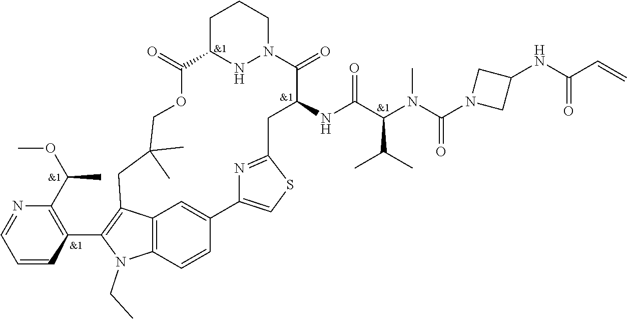 |
| BB294 | 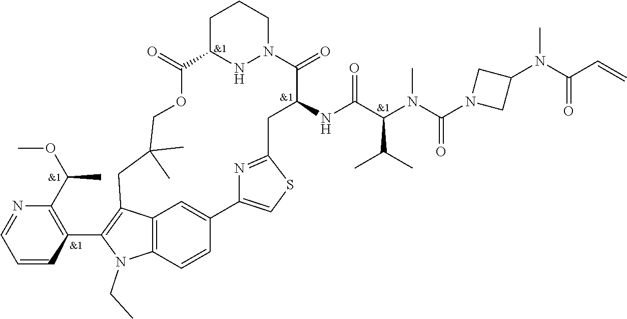 |
| BB295 | 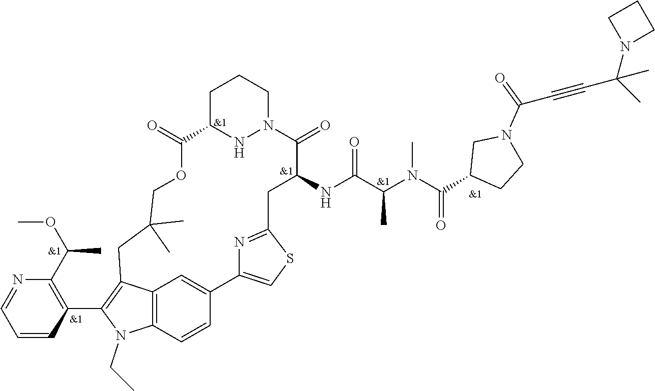 |
| BB296 | 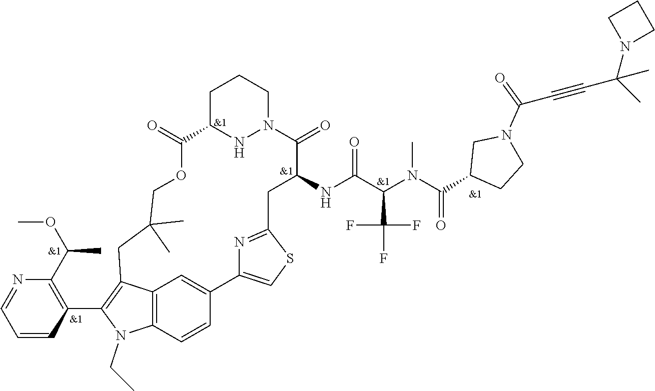 |
| BB297 | 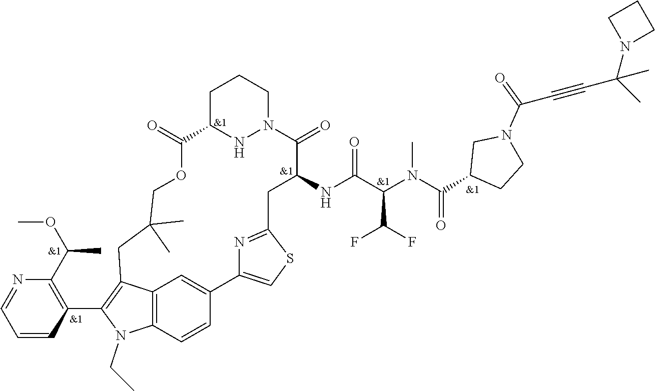 |
| BB298 | 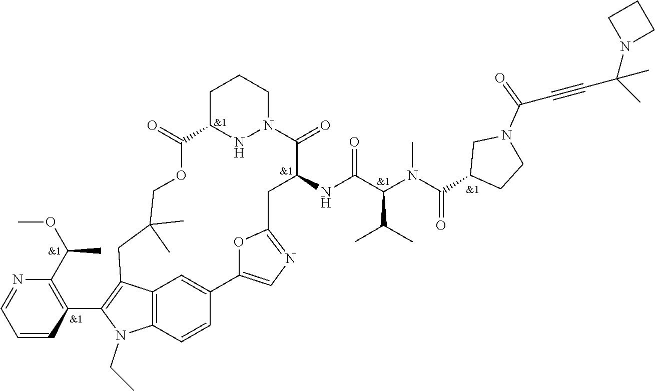 |
| BB299 | 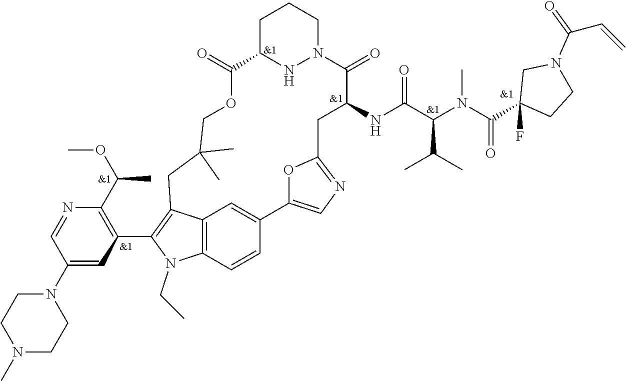 |
| BB300 | 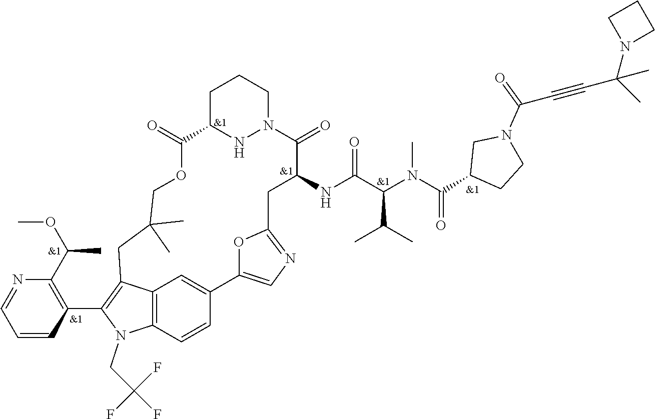 |
| BB301 | 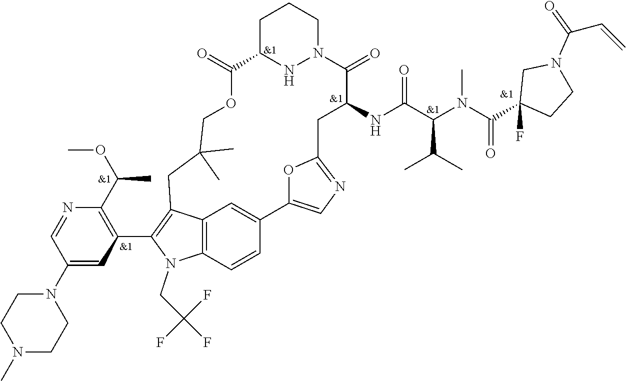 |
| BB302 | 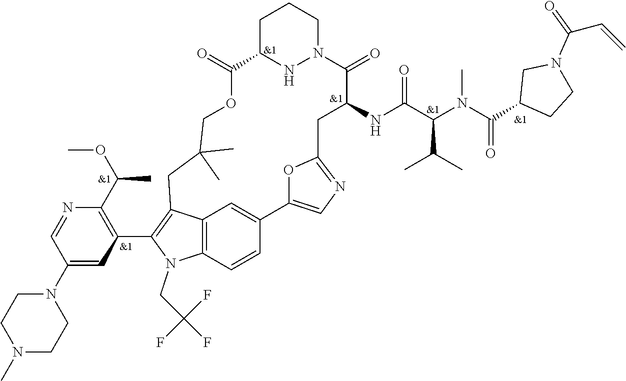 |
| BB303 | 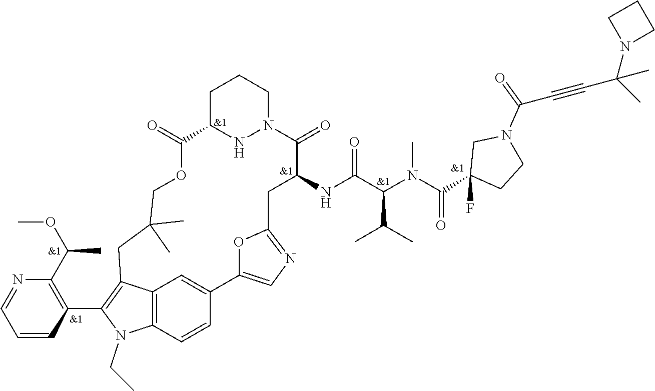 |
| BB304 | 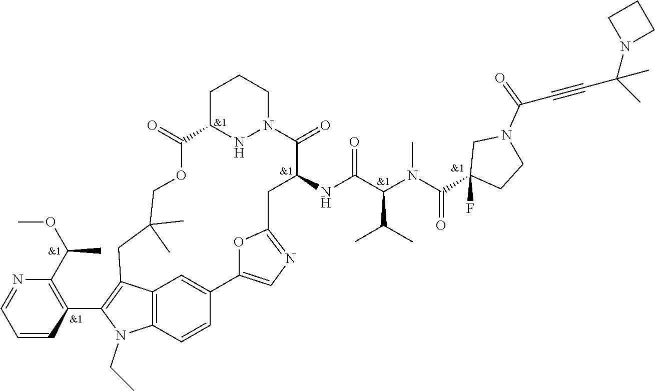 |
| BB305 | 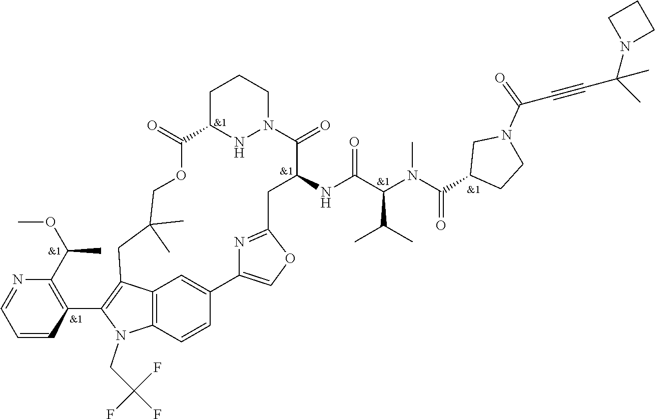 |
| BB306 | 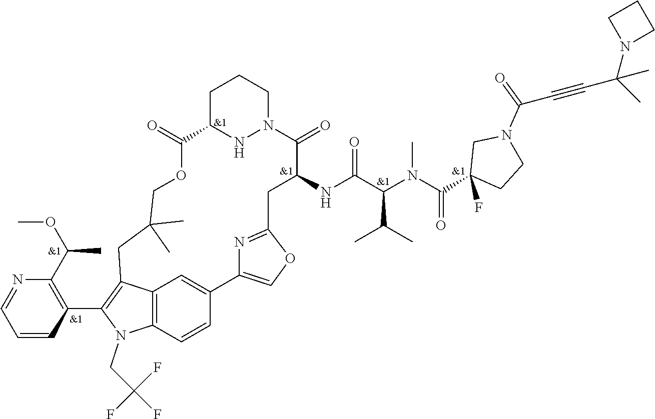 |
| BB307 | 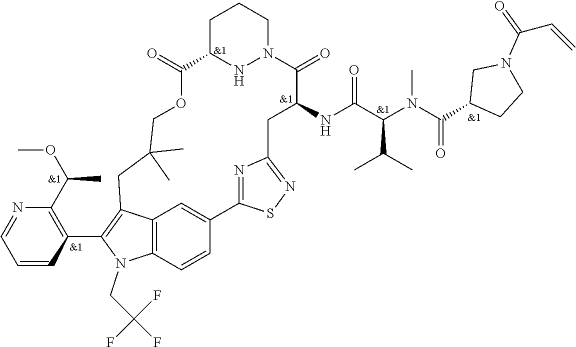 |
| BB308 | 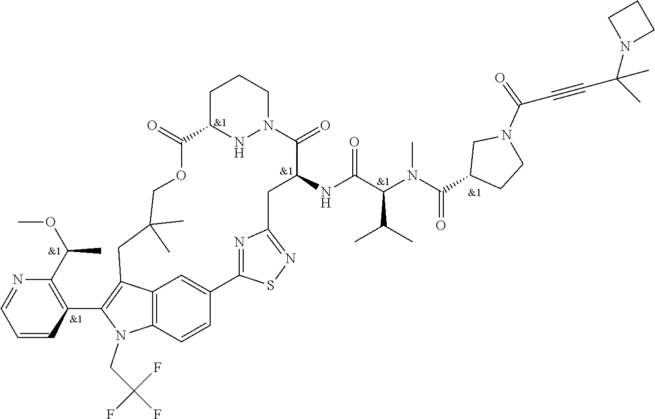 |
| BB309 | 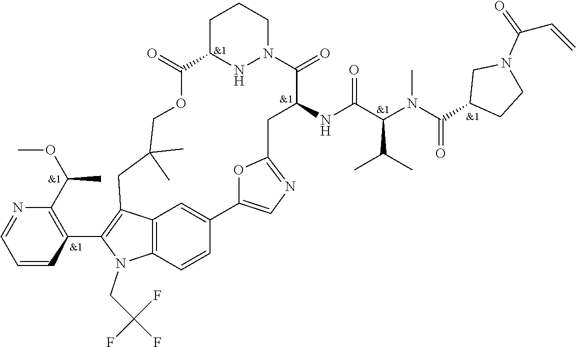 |
| BB310 | 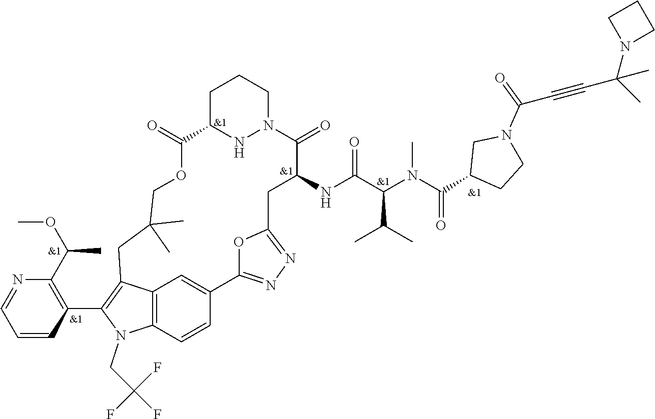 |
| BB311 | 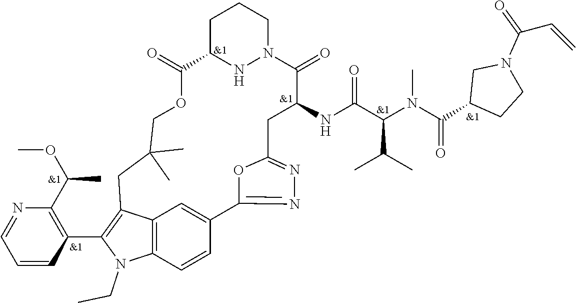 |
| BB312 | 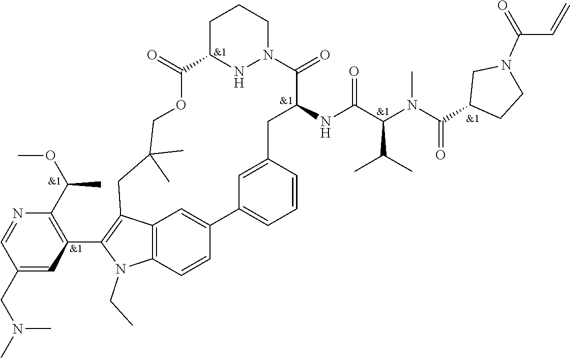 |
| BB313 | 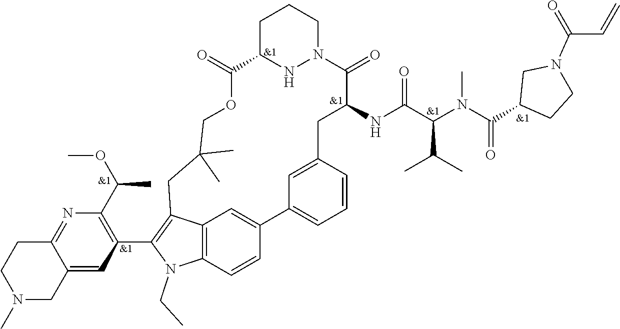 |
| BB314 | 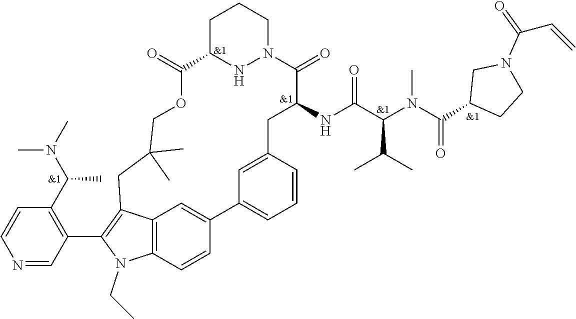 [SMILES: C=CC(=O)N1CC[C@H](C(=O)N(C)[C@H](C(=O)N[C@H]2Cc3cccc(c3)-c3ccc4c(c3)c(c(-c3cnccc3[C@@H](C)N(C)C)n4CC)CC(C)(C)COC(=O)[C@@H]3CCCN(N3)C2=O)C(C)C)C1] |
| BB315 | 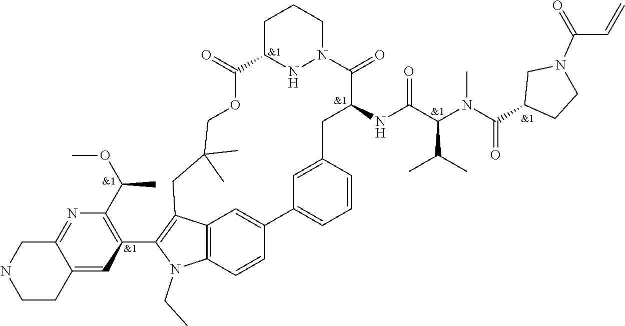 |
| BB316 | 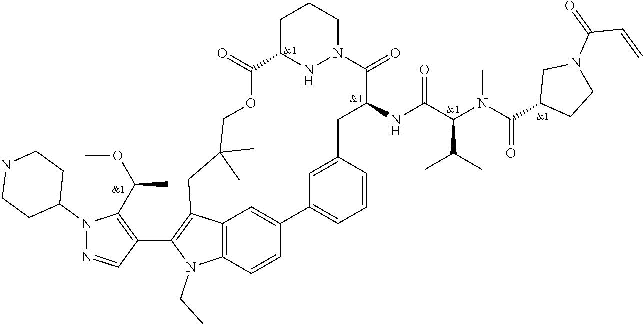 [SMILES: C=CC(=O)N1CC[C@H](C(=O)N(C)[C@H](C(=O)N[C@H]2Cc3cccc(c3)-c3ccc4c(c3)c(c(-c3cnn(C5CCNCC5)c3[C@H](C)OC)n4CC)CC(C)(C)COC(=O)[C@@H]3CCCN(N3)C2=O)C(C)C)C1] |
| BB317 | 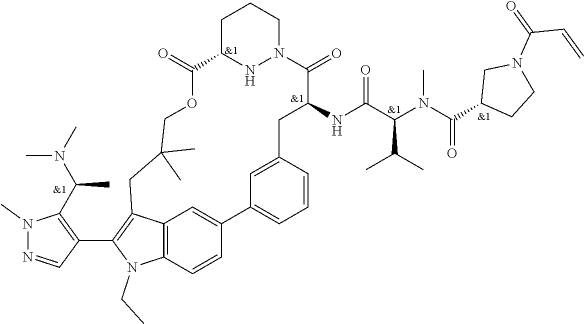 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)N(C)C)n4CC)CC(C)(C)COC(=O)[C@@H]3CCCN(N3)C2=O)C(C)C)C1] |
| BB318 | 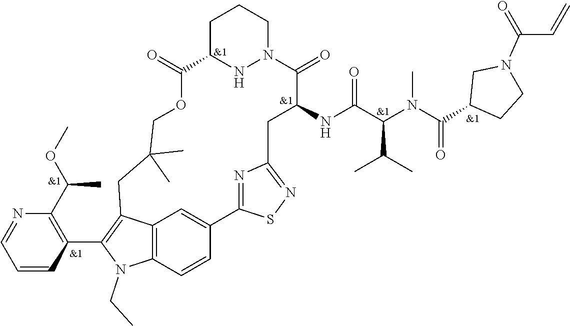 |
| BB319 | 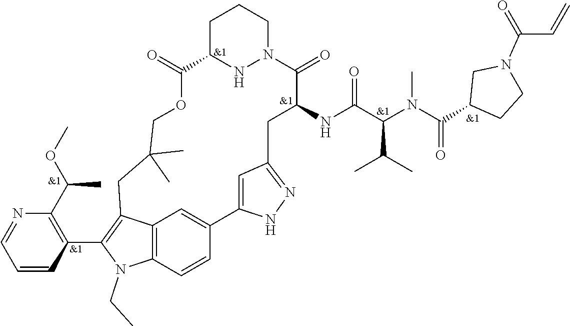 |
| BB320 | 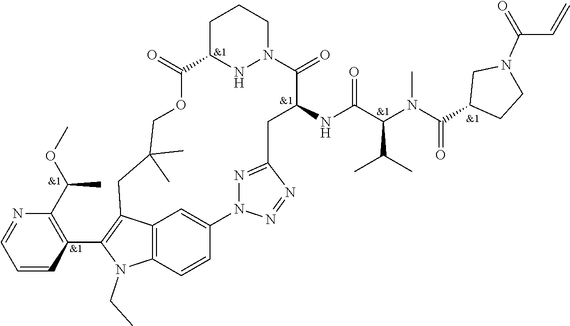 |
| BB321 | 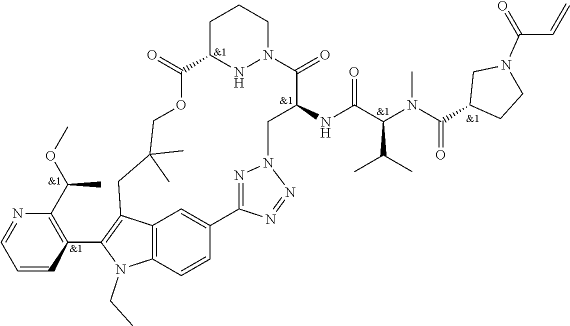 |
| BB322 | 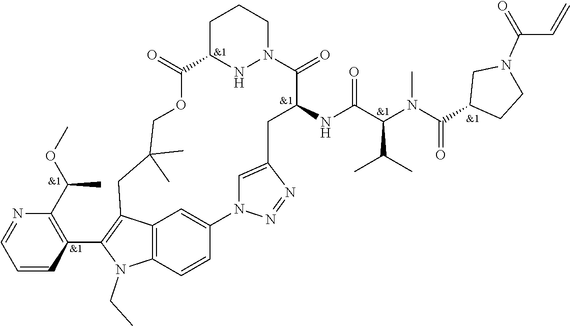 |
| BB323 | 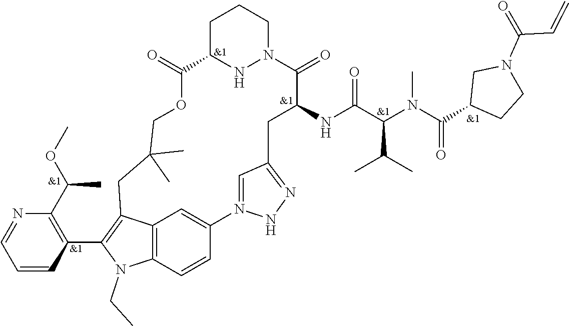 |
| BB324 | 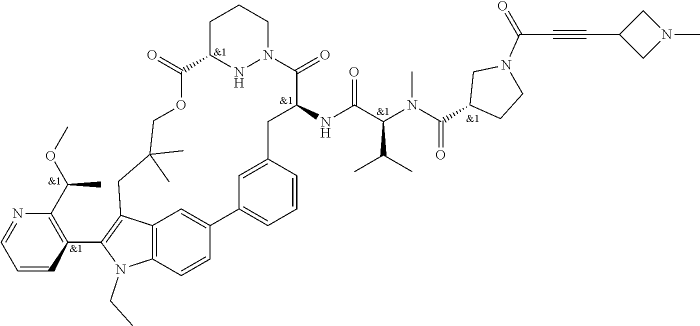 |
| BB325 | 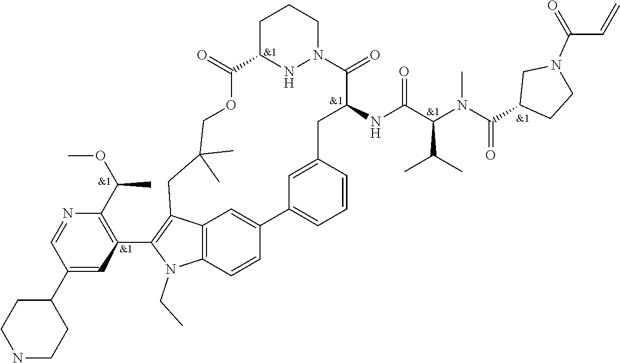 |
| BB326 | 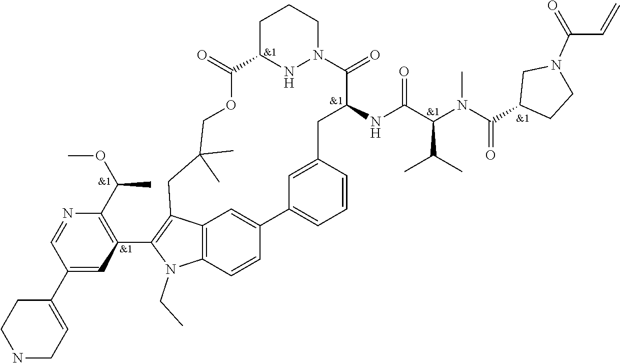 |
| BB327 | 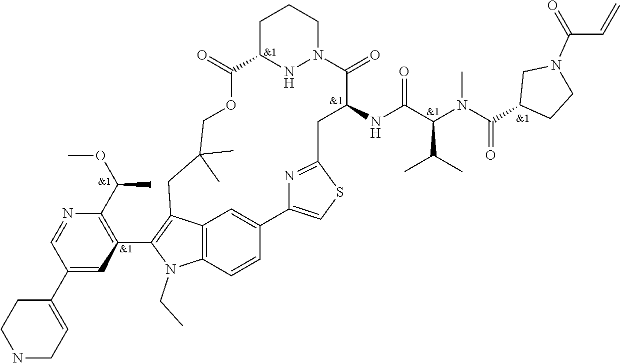 |
| BB328 | 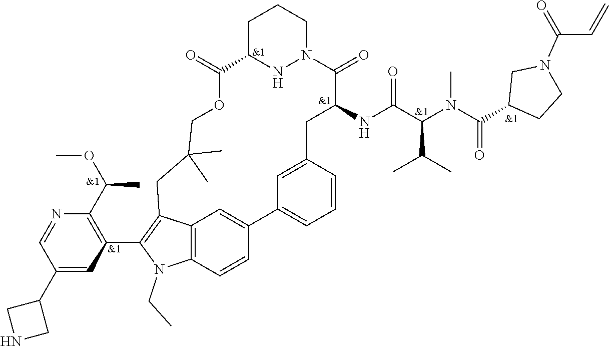 |
| BB329 | 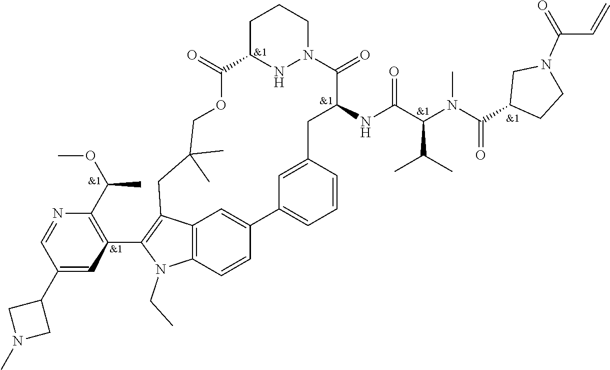 |
| BB330 | 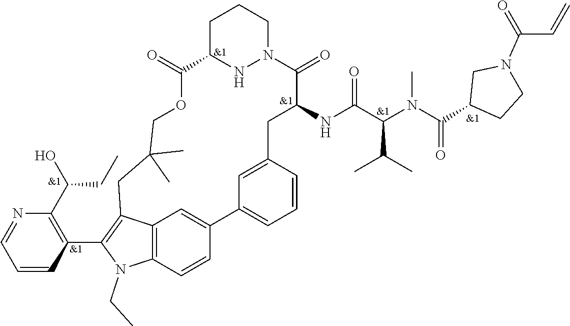 |
| BB331 | 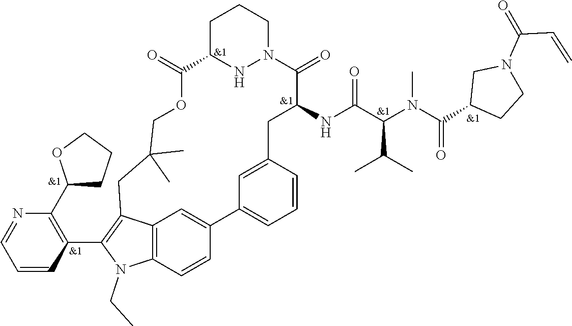 |
| BB332 | 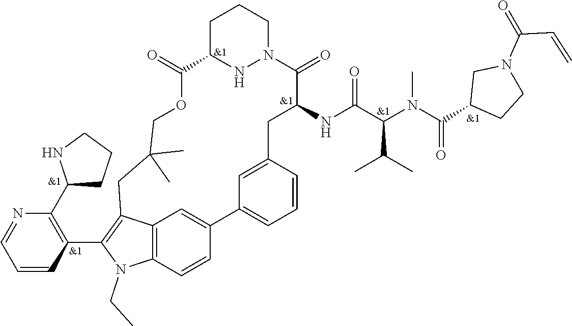 |
| BB333 | 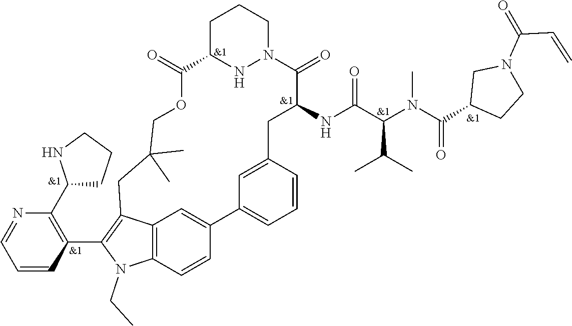 |
| BB334 | 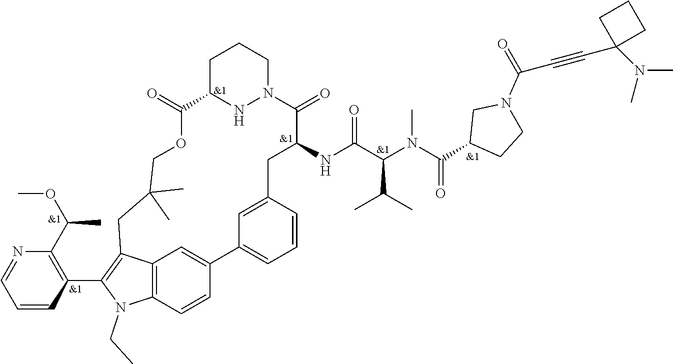 |
| BB335 | 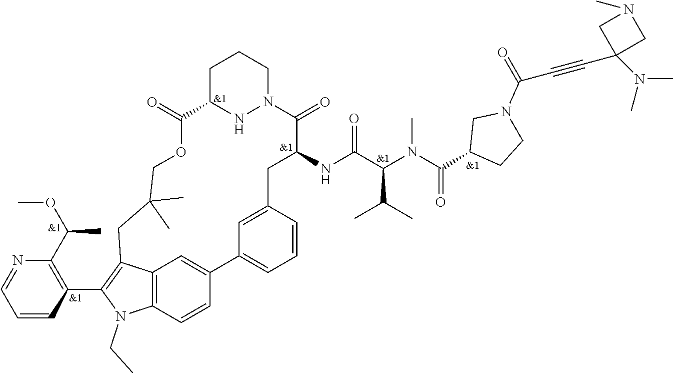 |
| BB336 | 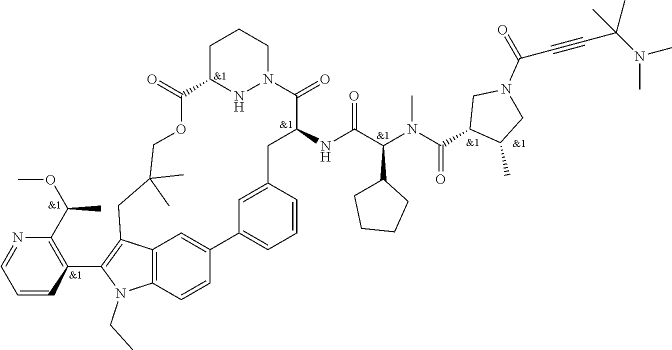 |
| BB337 | 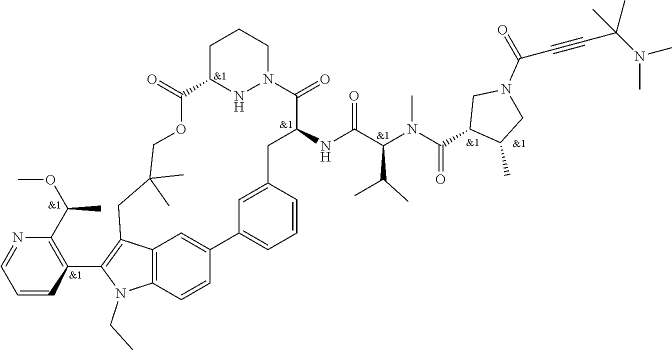 |
| BB338 | 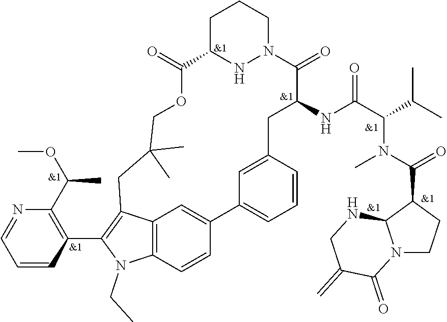 |
| BB339 | 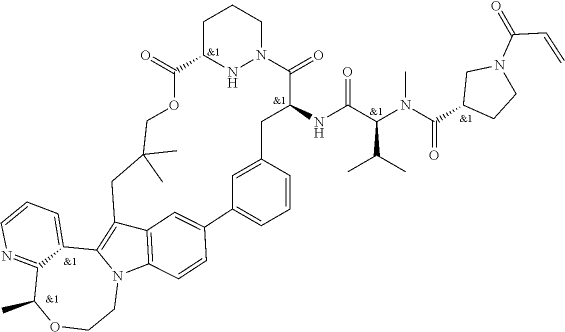 |
| BB340 | 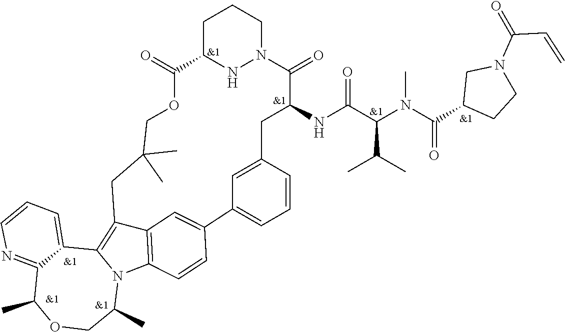 |
| BB341 | 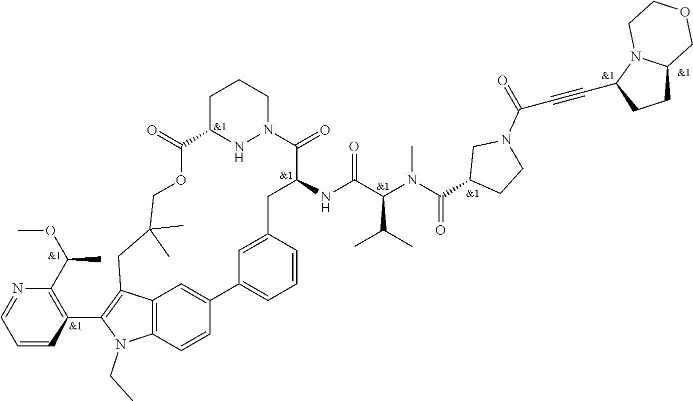 |
| BB342 | 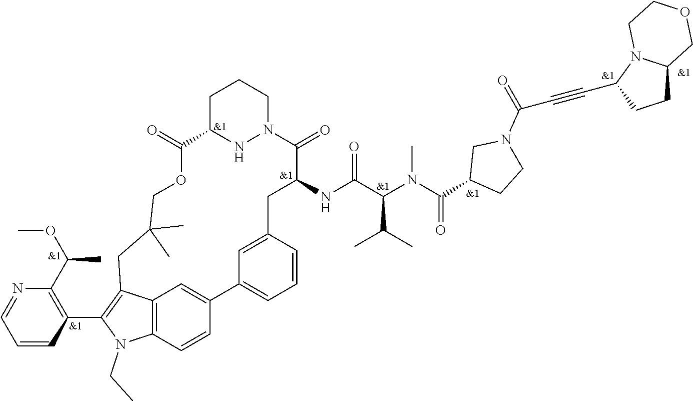 |
| BB343 | 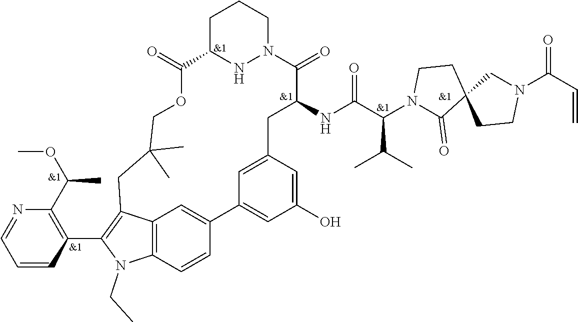 |
| BB344 | 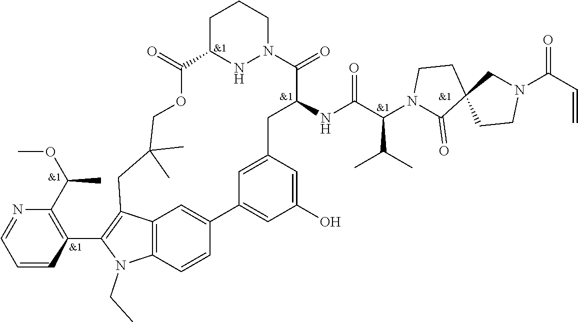 |
| BB345 | 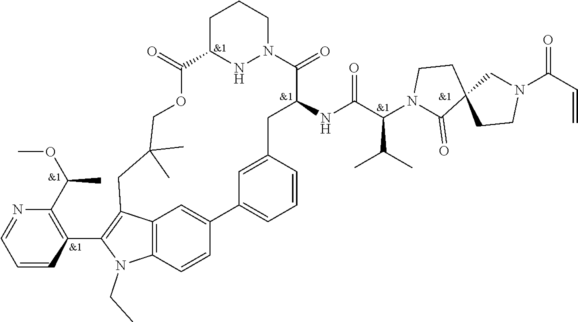 |
| BB346 | 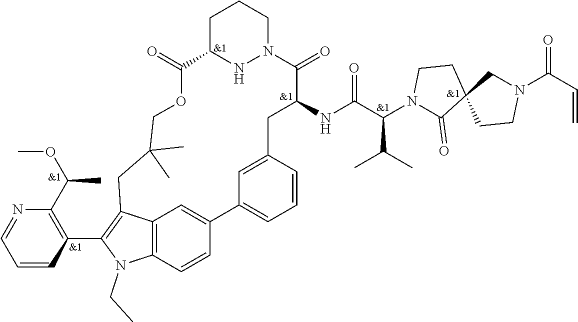 |
| BB347 | 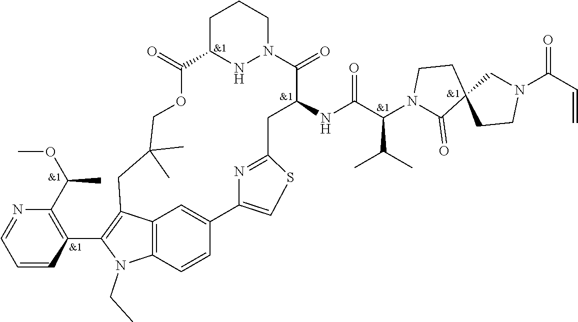 |
| BB348 | 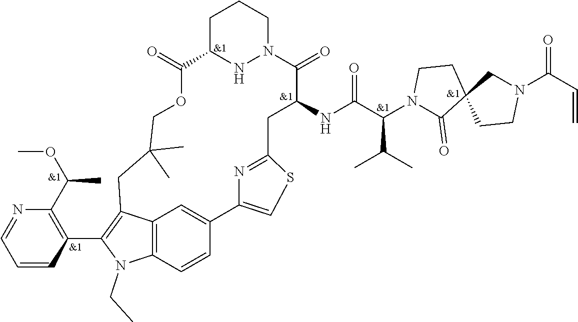 |
| BB349 | 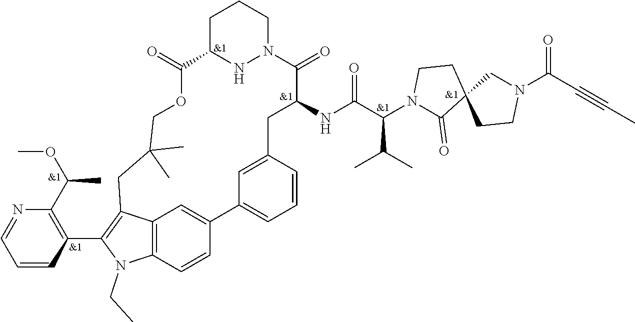 |
| BB350 | 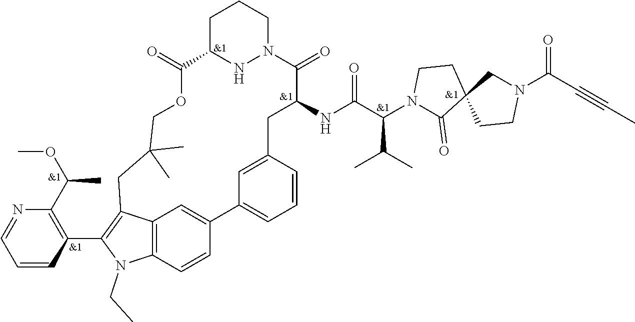 |
| BB351 | 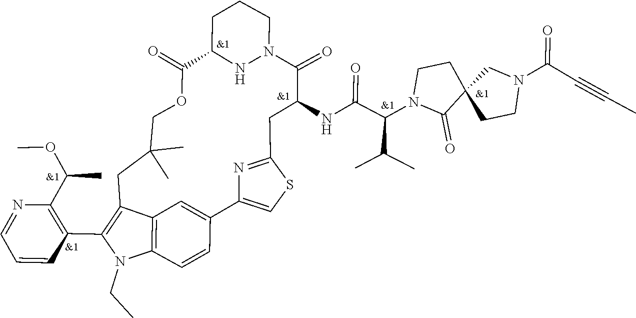 |
| BB352 | 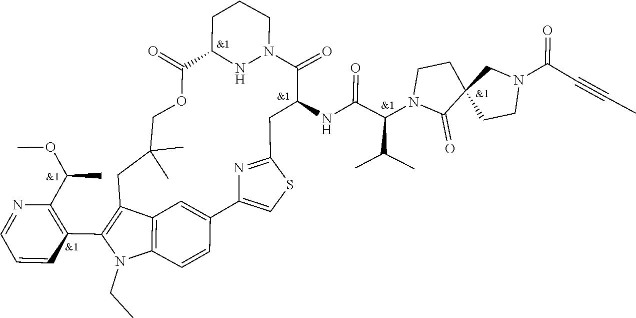 |
| BB353 | 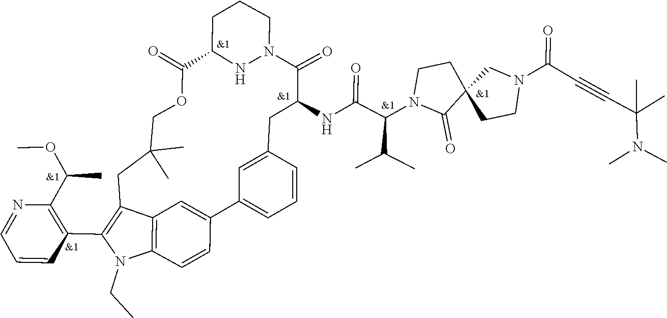 |
| BB354 | 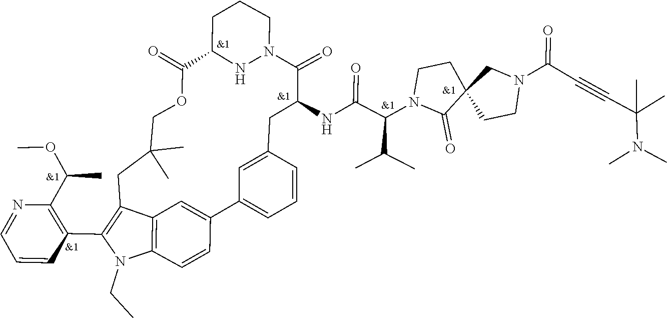 |
| BB355 | 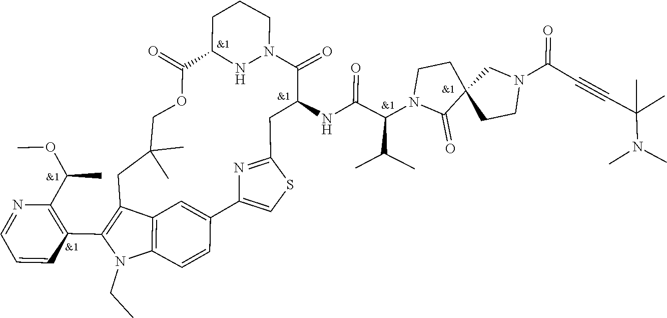 |
| BB356 | 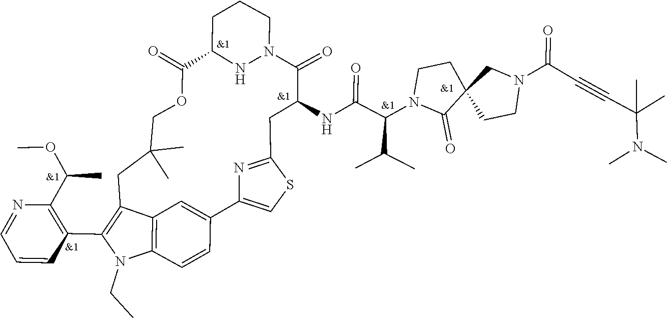 |
| BB357 | 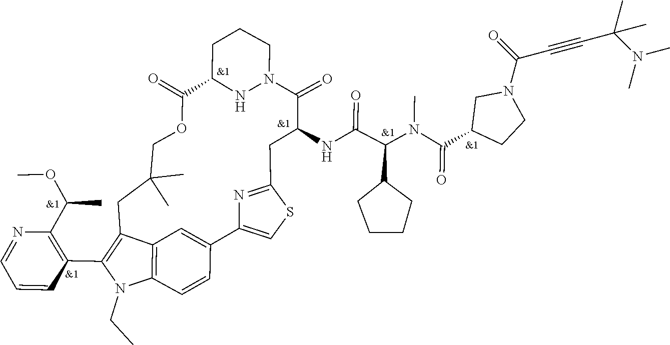 |
| BB358 | 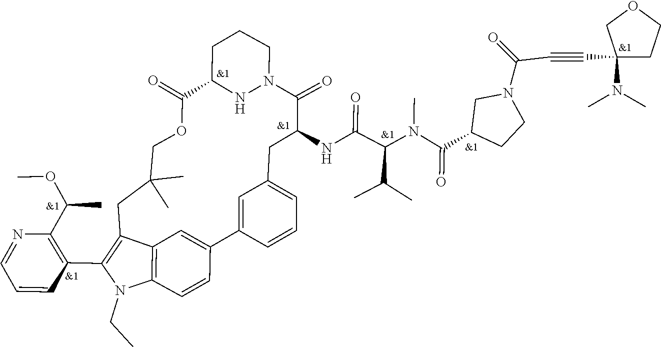 |
| BB359 | 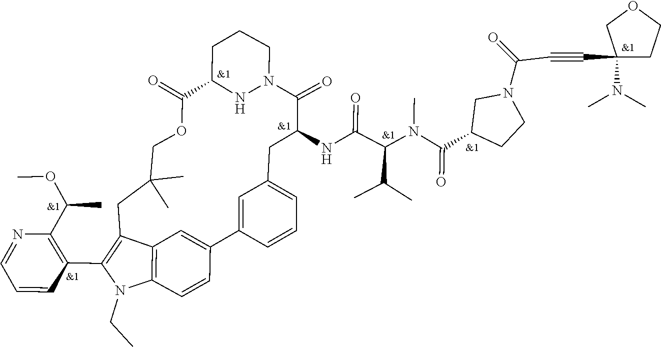 |
| BB360 | 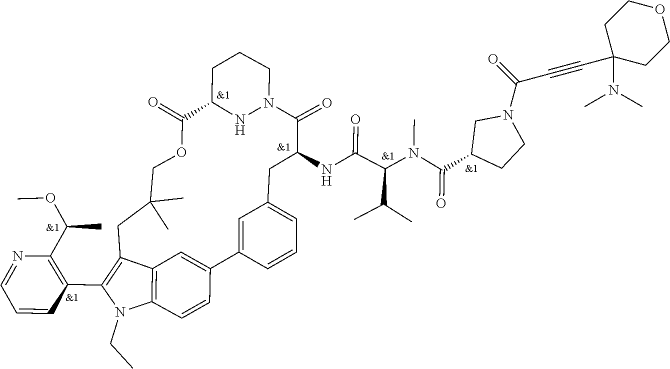 |
| BB361 | 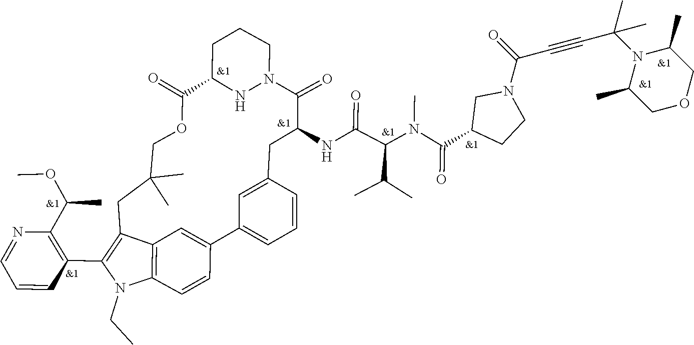 |
| BB362 | 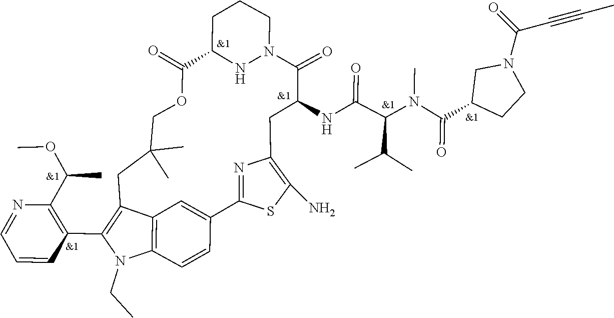 |
| BB363 | 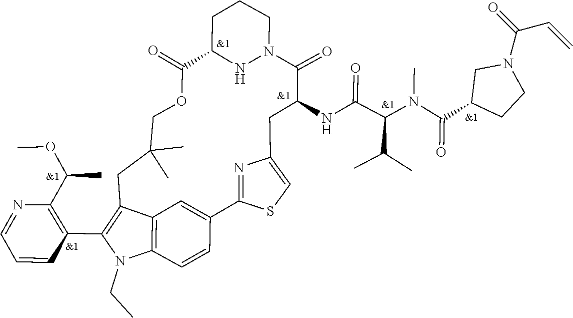 |
| BB364 | 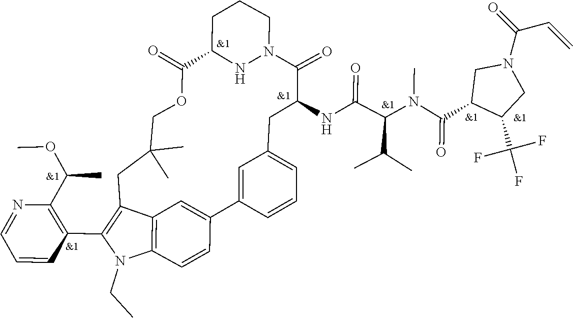 |
| BB365 | 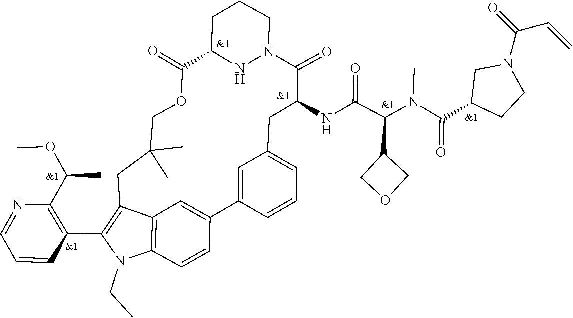 |
| BB366 | 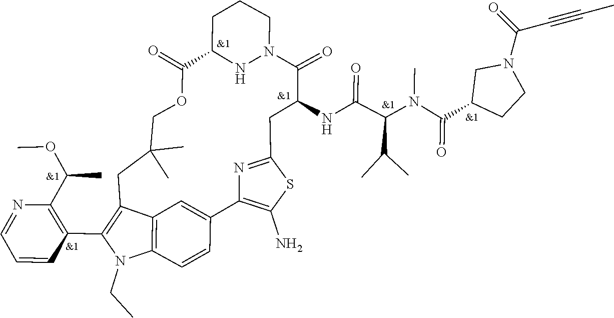 |
| BB367 | 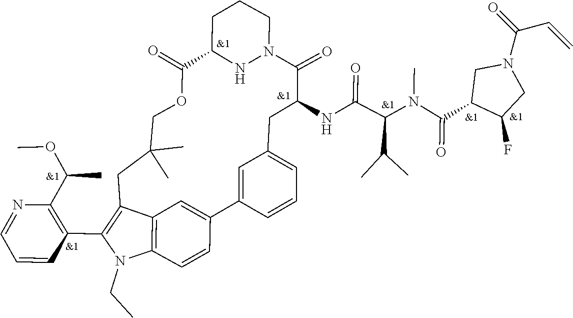 |
| BB368 | 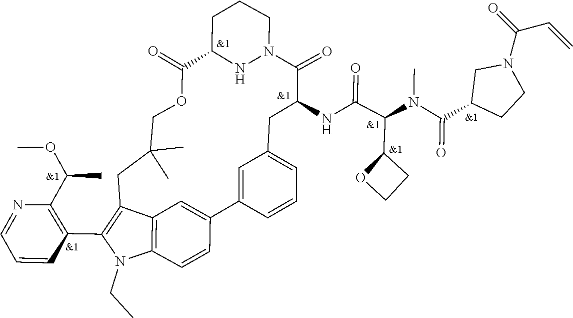 |
| BB369 | 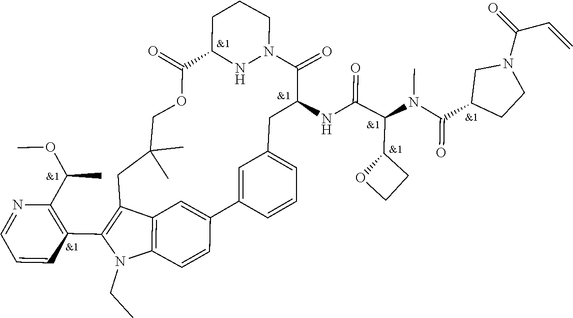 |
| BB370 | 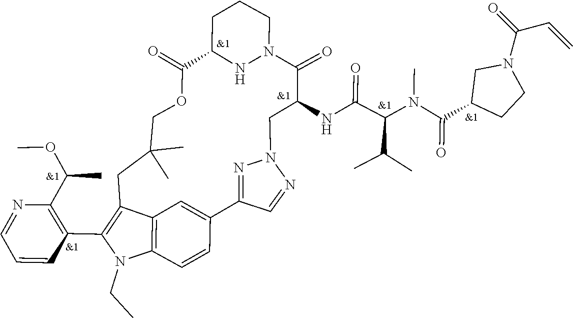 |
| BB371 | 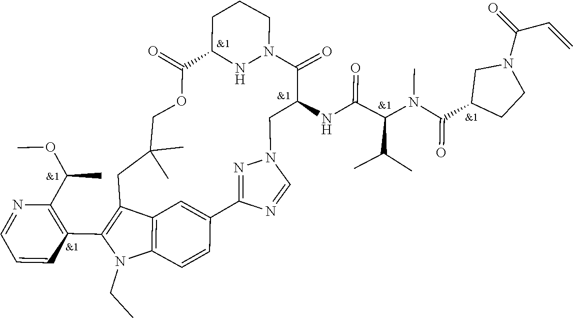 |
| BB372 | 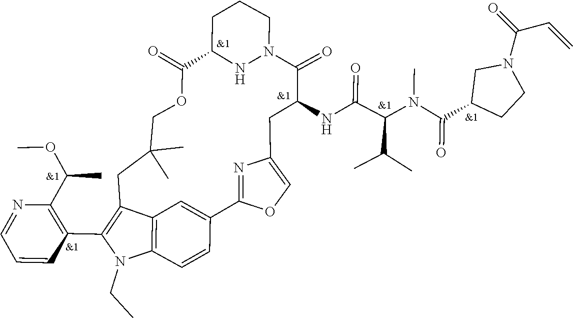 |
| BB373 | 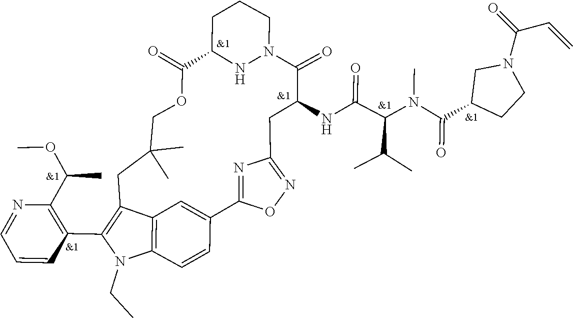 |
| BB374 | 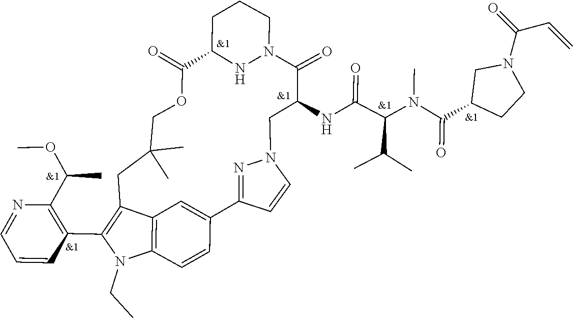 |
| BB375 | 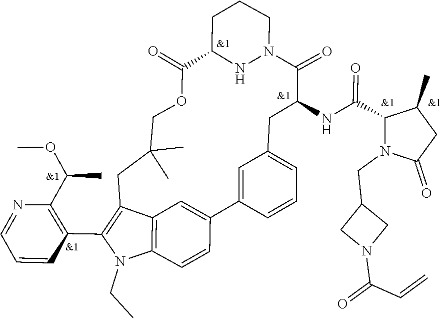 |
| BB376 | 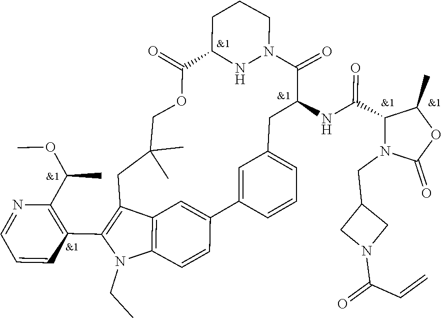 |
| BB377 | 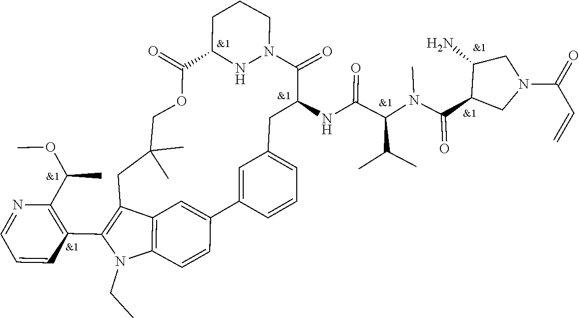 |
| BB378 | 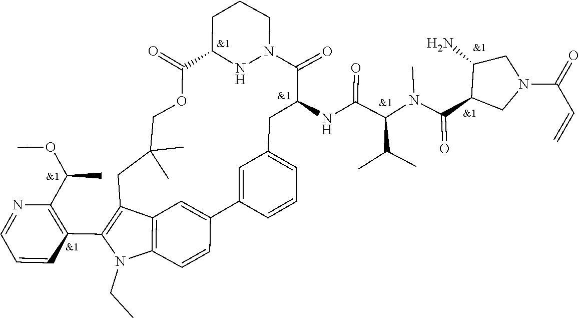 |
| BB379 | 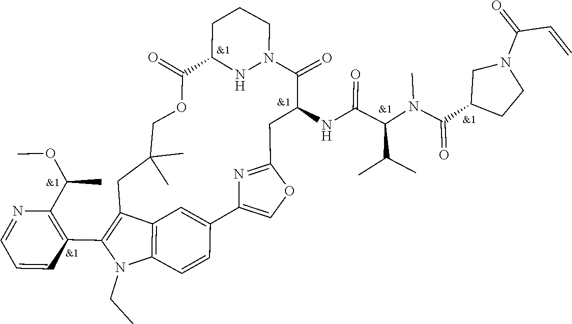 |
| BB380 | 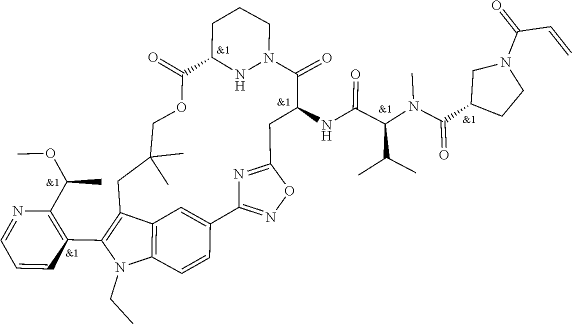 |
| BB381 | 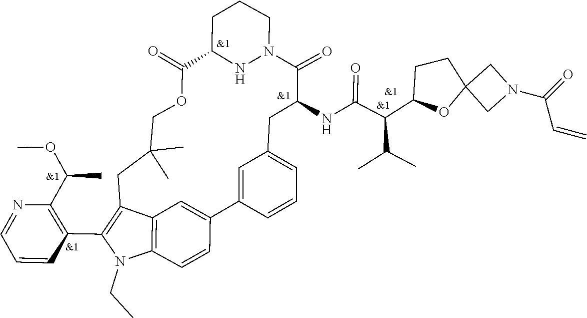 |
| BB382 | 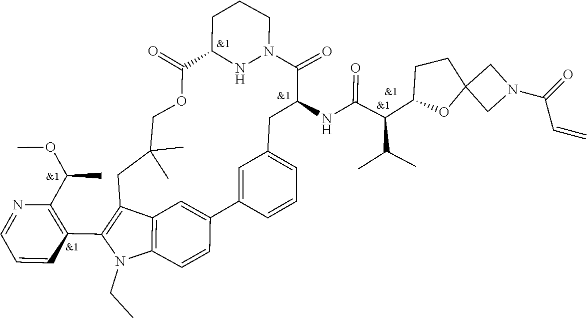 |
| BB383 | 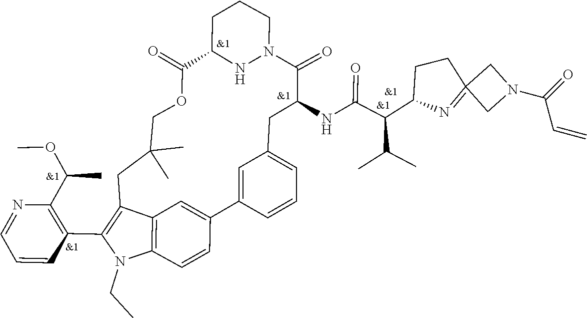 |
| BB384 | 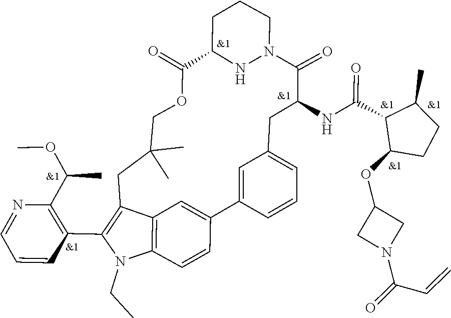 |
| BB385 | 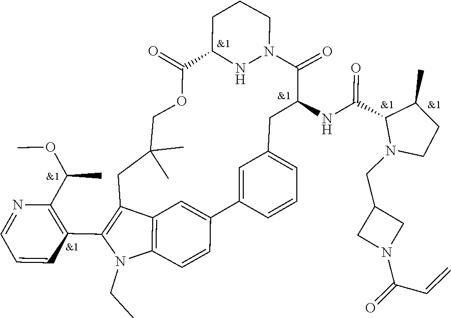 |
| BB386 | 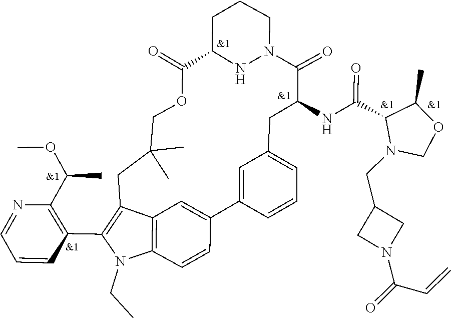 |
| BB387 | 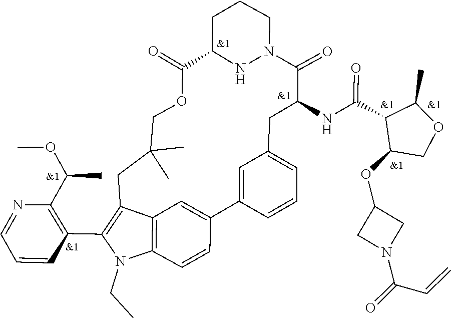 |
| BB388 | 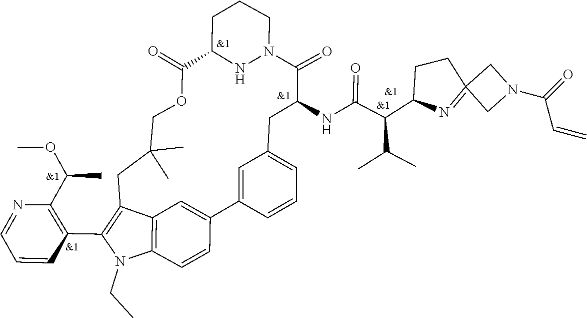 |
| BB389 | 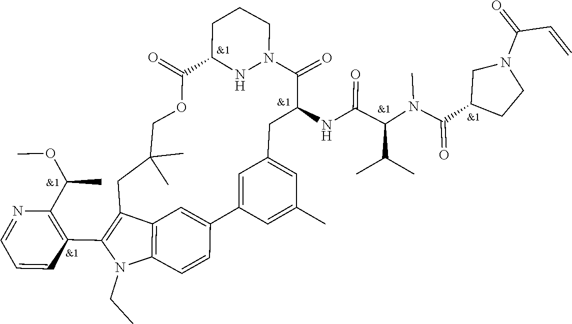 |
| BB390 | 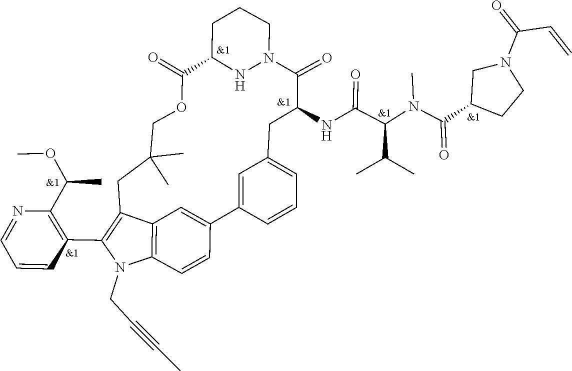 |
| BB391 | 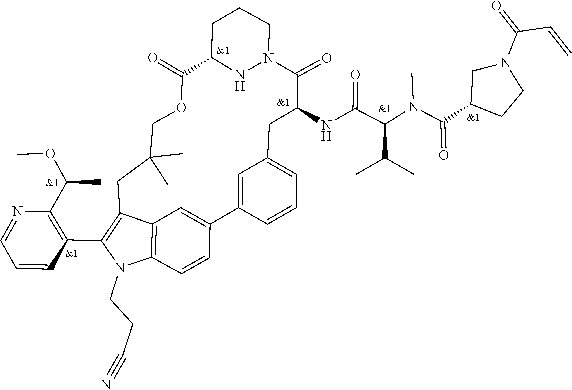 |
| BB392 | 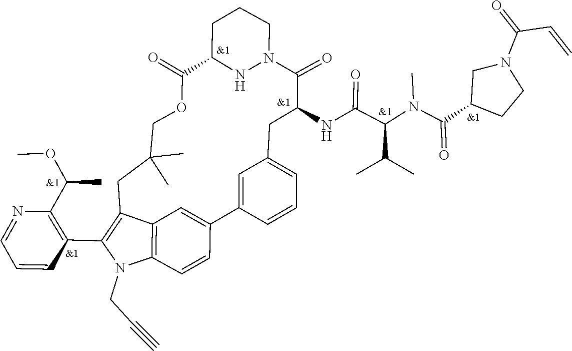 |
| BB393 | 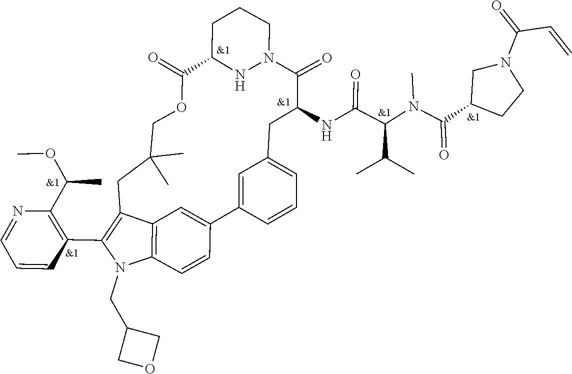 |
| BB394 | 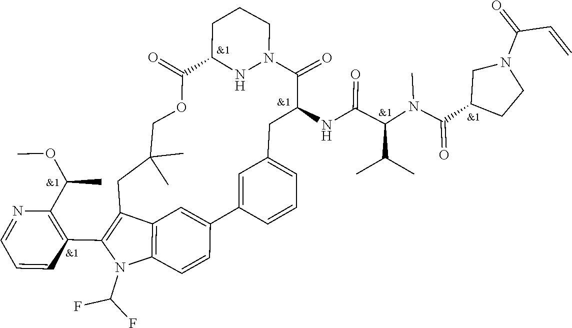 |
| BB395 | 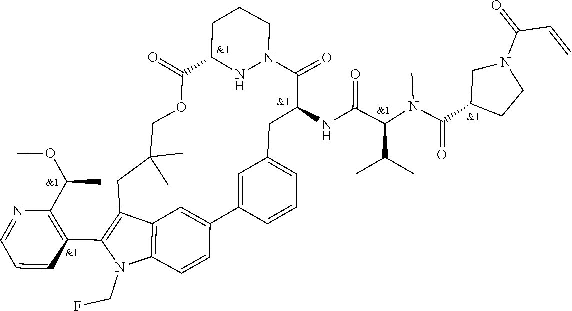 |
| BB396 | 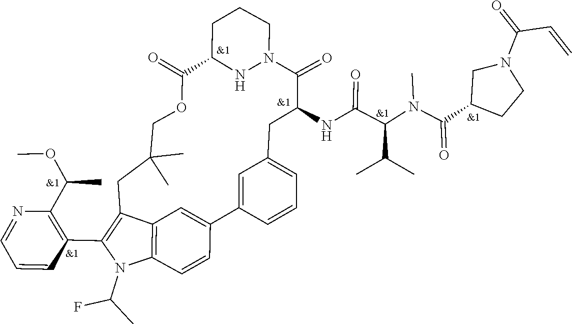 |
| BB397 | 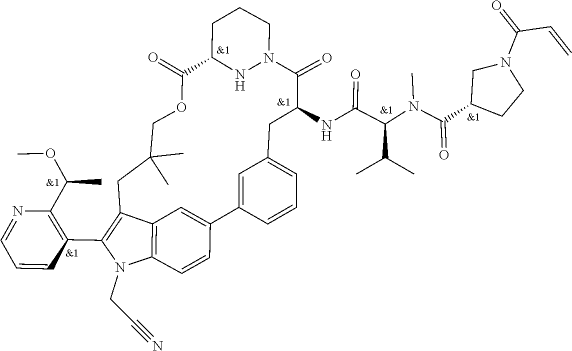 |
| BB398 | 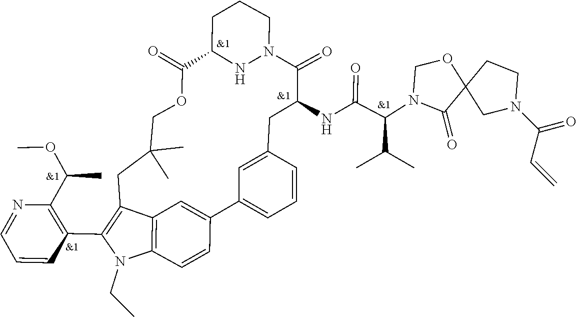 |
| BB399 | 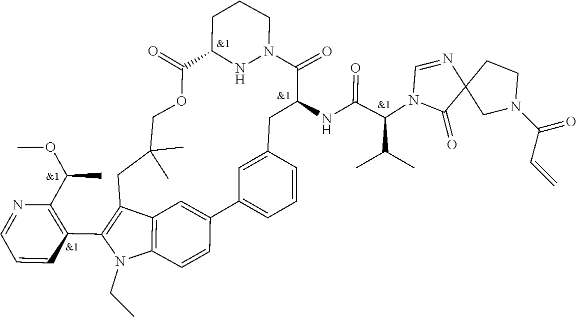 |
| BB400 | 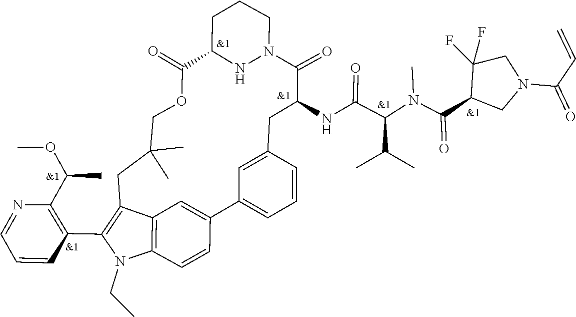 |
| BB401 | 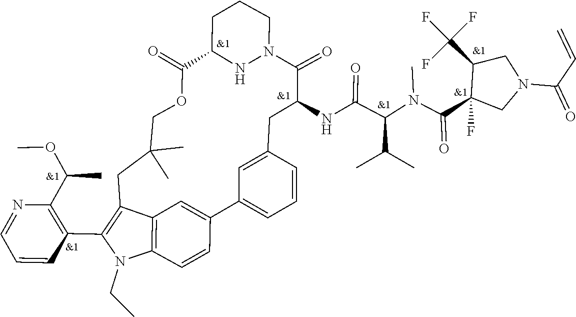 |
| BB402 | 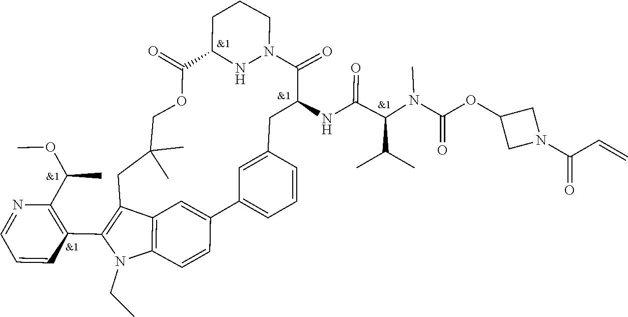 |
| BB403 | 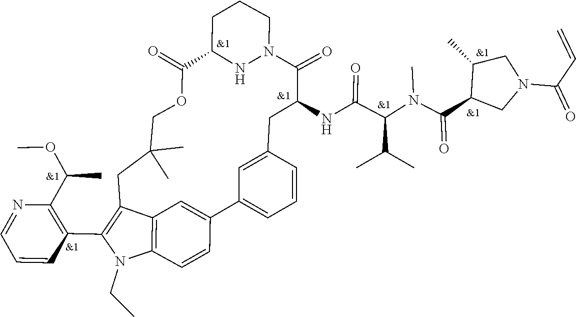 |
| BB404 | 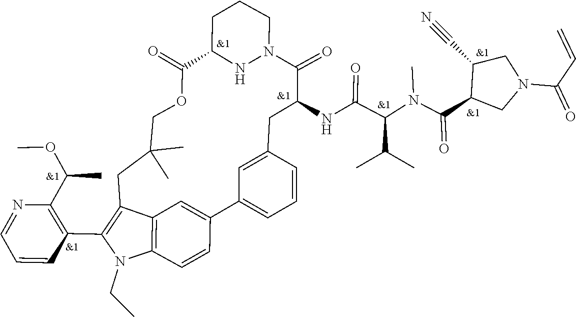 |
| BB405 | 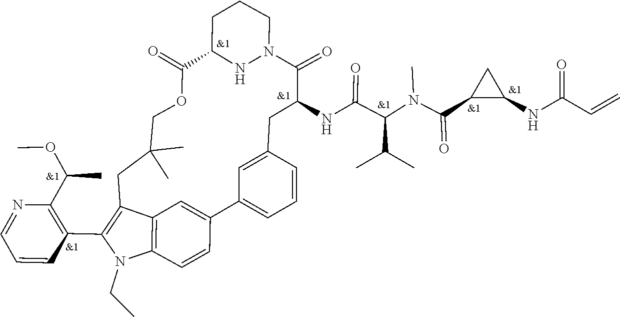 |
| BB406 | 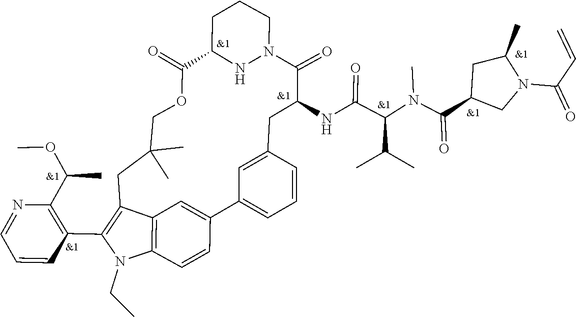 |
| BB407 | 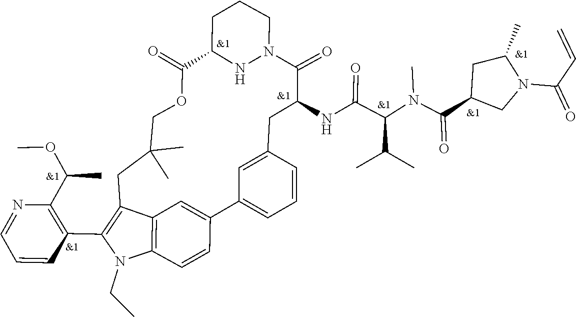 |
| BB408 | 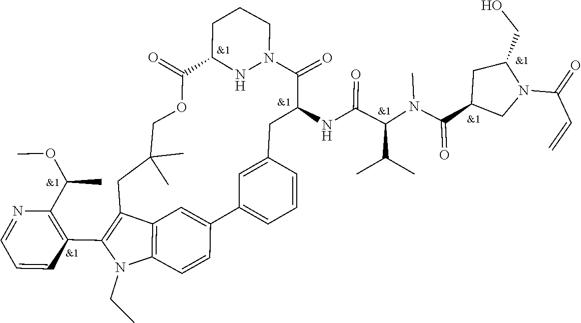 |
| BB409 | 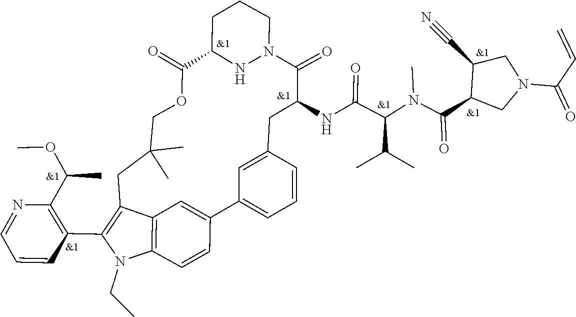 |
| BB410 | 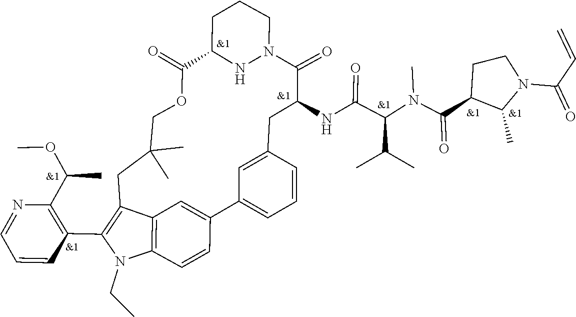 |
| BB411 | 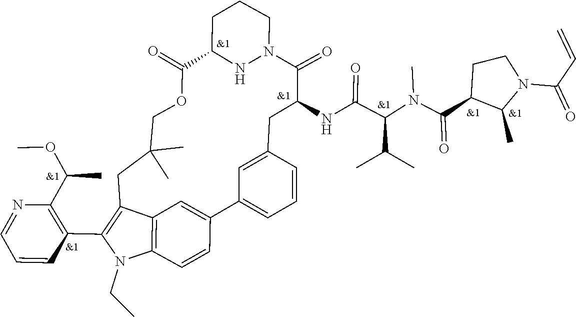 |
| BB412 | 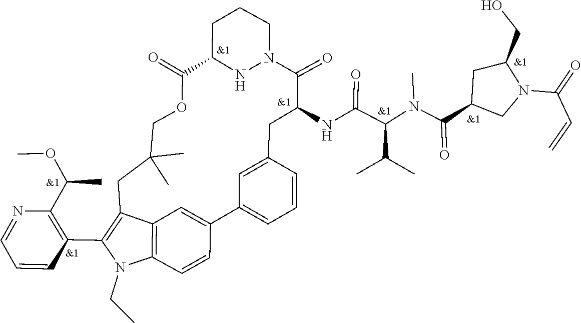 |
| BB413 | 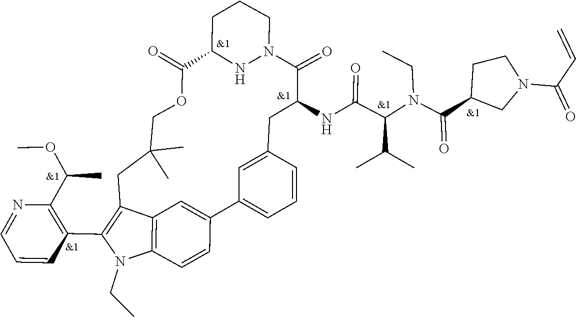 |
| BB414 | 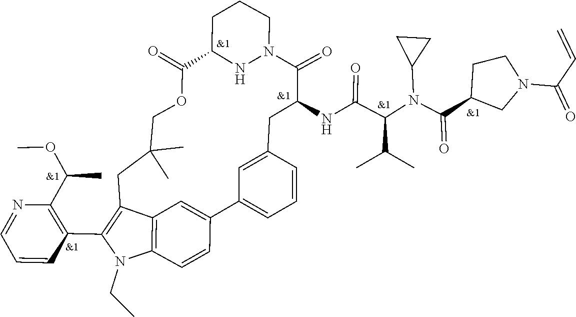 |
| BB415 | 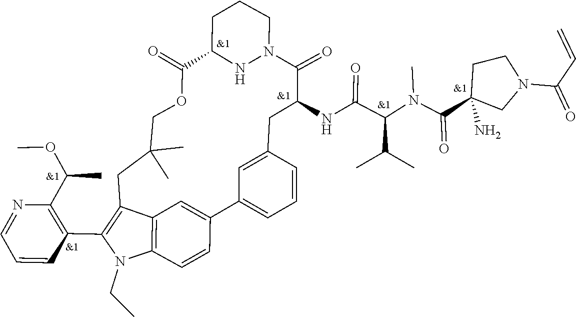 |
| BB416 | 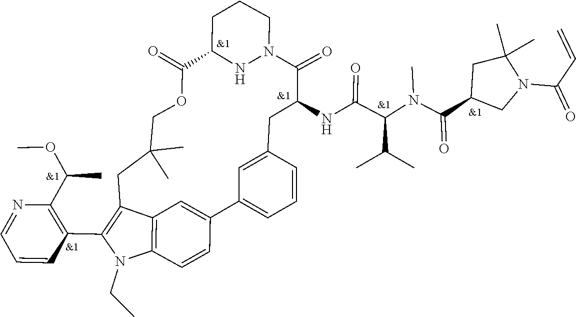 |
| BB417 | 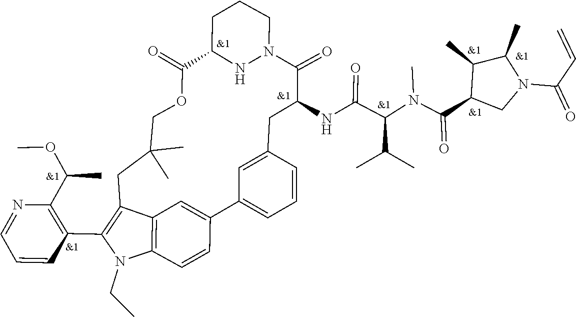 |
| BB418 | 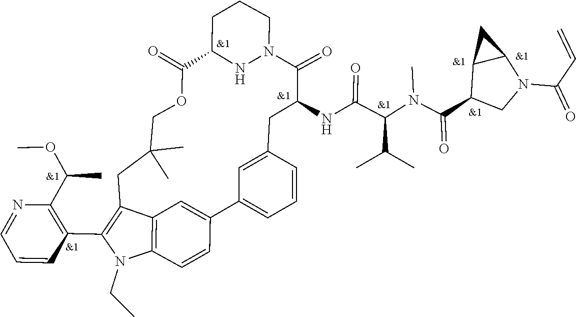 |
| BB419 | 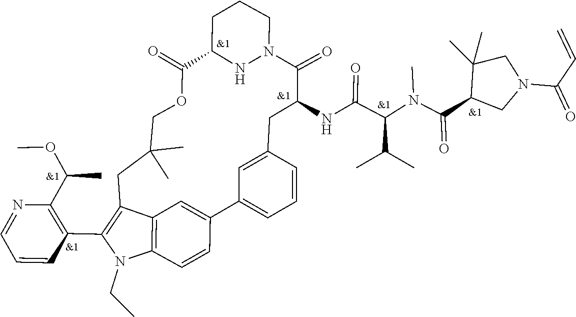 |
| BB420 | 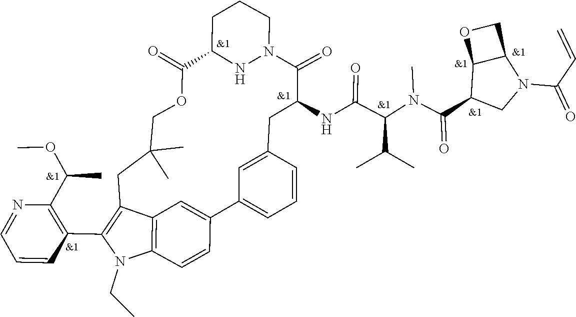 |
| BB421 | 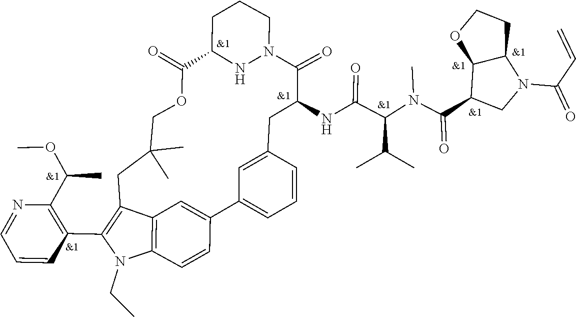 |
| BB422 | 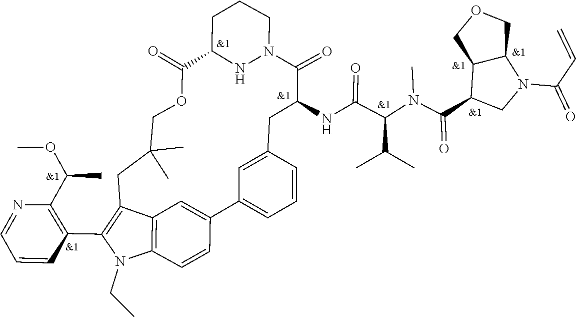 |
| BB423 | 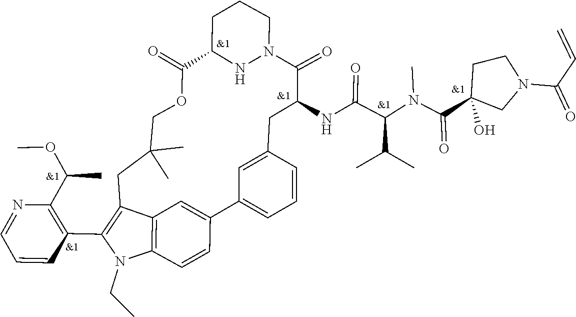 |
| BB424 | 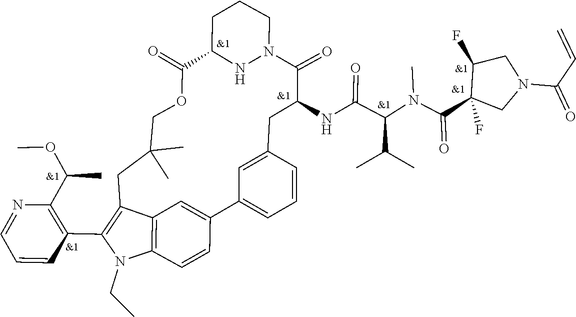 |
| BB425 | 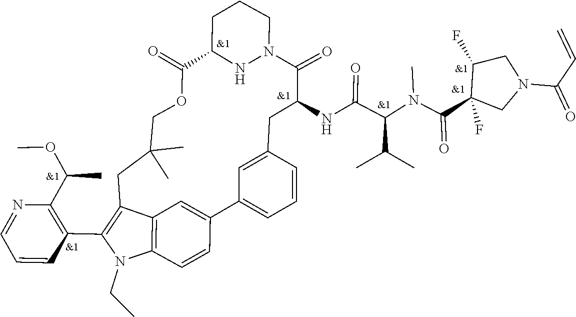 |

Note that some compounds are shown with bonds as flat or wedged. In some instances, the relative stereochemistry of stereoisomers has
been determined; in some instances, the absolute stereochemistry has been determined. All stereoisomers of the compounds of the foregoing
table are contemplated by the present invention. In particular embodiments, an atropisomer of a compound of the foregoing table is cotemplated.

In some embodiments, the RAS(ON) inhibitor is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

In some embodiments, the RAS(ON) inhibitor is provided as a conjugate, or salt thereof, comprising the structure of Formula BIV:

M-L-P Formula BIV
wherein L is a linker;
P is a monovalent organic moiety; and
M has the structure of Formula BVa:

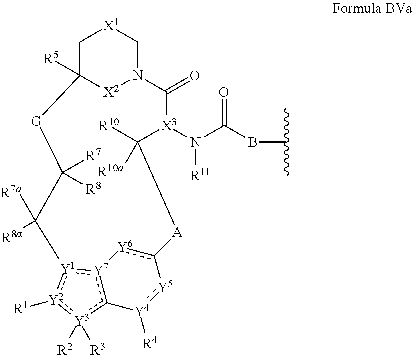

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments the conjugate, or salt thereof, comprises the structure of Formula BIV:

M-L-P Formula BIV
wherein L is a linker;
P is a monovalent organic moiety; and
M has the structure of Formula BVb:

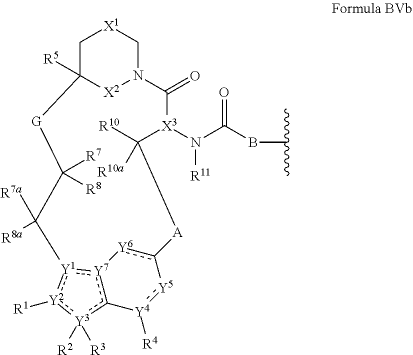

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N; Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the conjugate has the structure of Formula BIV:

M-L-P Formula BIV
wherein L is a linker;
P is a monovalent organic moiety; and
M has the structure of Formula BVc:

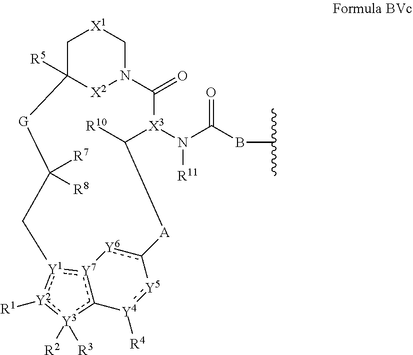

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, the RAS(ON) inhibitor has the structure of Formula BIV:

M-L-P Formula BIV
wherein L is a linker;
P is a monovalent organic moiety; and
M has the structure of Formula BVd:

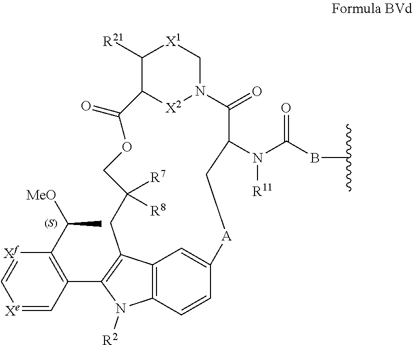

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of formula BI and subformula thereof, X^{e} is N and X^{f} is CH. In some embodiments, X^{e} is CH and X^{f} is N.

In some embodiments, the RAS(ON) inhibitor has the structure of of Formula BIV:

M-L-P Formula BIV
wherein L is a linker;
P is a monovalent organic moiety; and
M has the structure of Formula BVe:

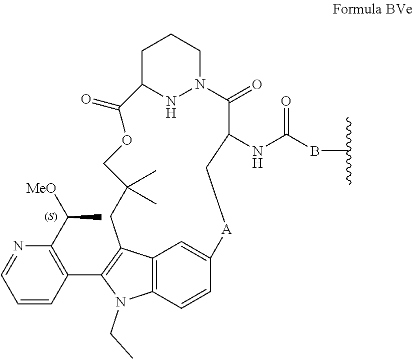

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of a conjugate of Formula BIV, the linker has the structure of Formula BII:

A¹-(B¹)_{f}—(C¹)_{g}—(B²)ₕ-(D¹)-(B³)ᵢ—(C²)ⱼ—(B⁴)ₖ-A² Formula BII
where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)_{J}—(B⁴)ₖ-A².

In some embodiments of a conjugate of formula BIV, the monovalent organic moiety is a protein, such as a Ras protein. In some embodiments, the Ras protein is K-Ras G12C, K-Ras G13C, H-Ras G12C, H-Ras G13C, N-Ras G12C, or N-Ras G13C. Other Ras proteins are described herein. In some embodiments, the linker is bound to the monovalent organic moiety through a bond to a sulfhydryl group of an amino acid residue of the monovalent organic moiety. In some embodiments, the linker is bound to the monovalent organic moiety through a bond to a carboxyl group of an amino acid residue of the monovalent organic moiety.

The compounds described in Tables B1 and B2 may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below or as described in WO 2021/091982.

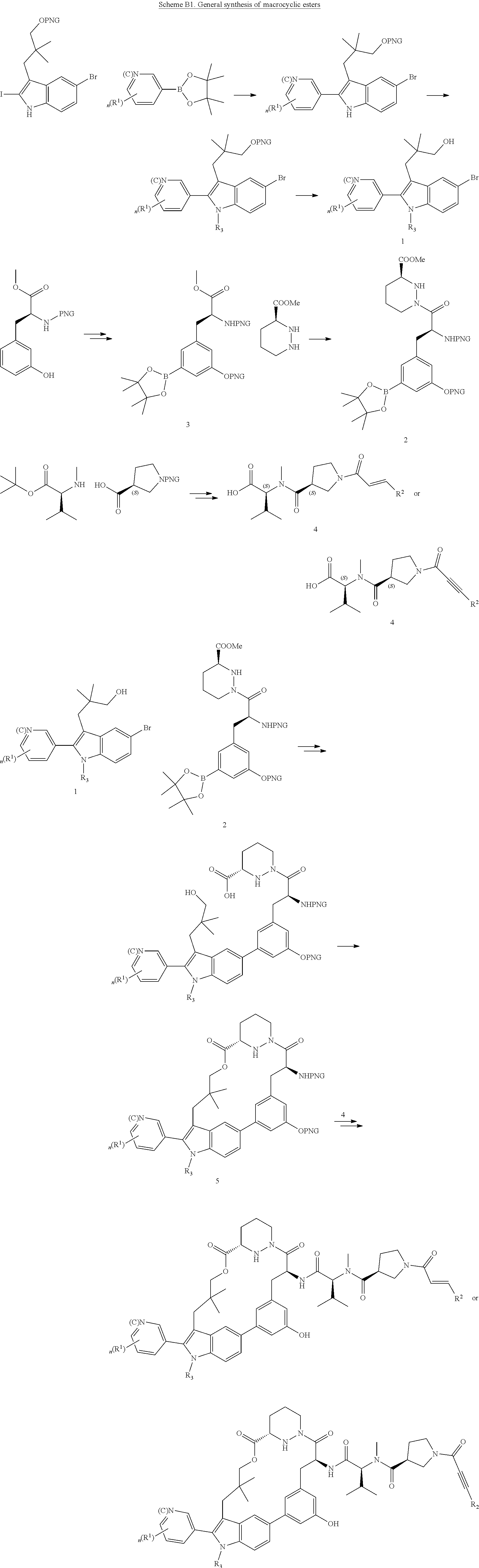

A general synthesis of macrocyclic esters is outlined in Scheme B1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions. Methyl-amino-hexahydropyridazine-3-carboxylate-boronic ester (2) can be prepared in three steps, including protection, iridium catalyst mediated borylation, and coupling with methyl methyl (S)-hexahydropyridazine-3-carboxylate.

An appropriately substituted acetylpyrrolidine-3-carbonyl-N-methyl-L-valine (or an alternative aminoacid derivative (4) can be made by coupling of methyl-L-valinate and protected (S)-pyrrolidine-3-carboxylic acid, followed by deprotection, coupling with a carboxylic acid containing an appropriately substituted Michael acceptor, and a hydrolysis step.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of a Pd catalyst followed by hydrolysis and macrolactonization steps to result in an appropriately protected macrocyclic intermediate (5). Deprotection and coupling with an appropriately substituted intermediate 4 results in a macrocyclic product. Additional deprotection and/or functionalization steps can be required to produce the final compound.

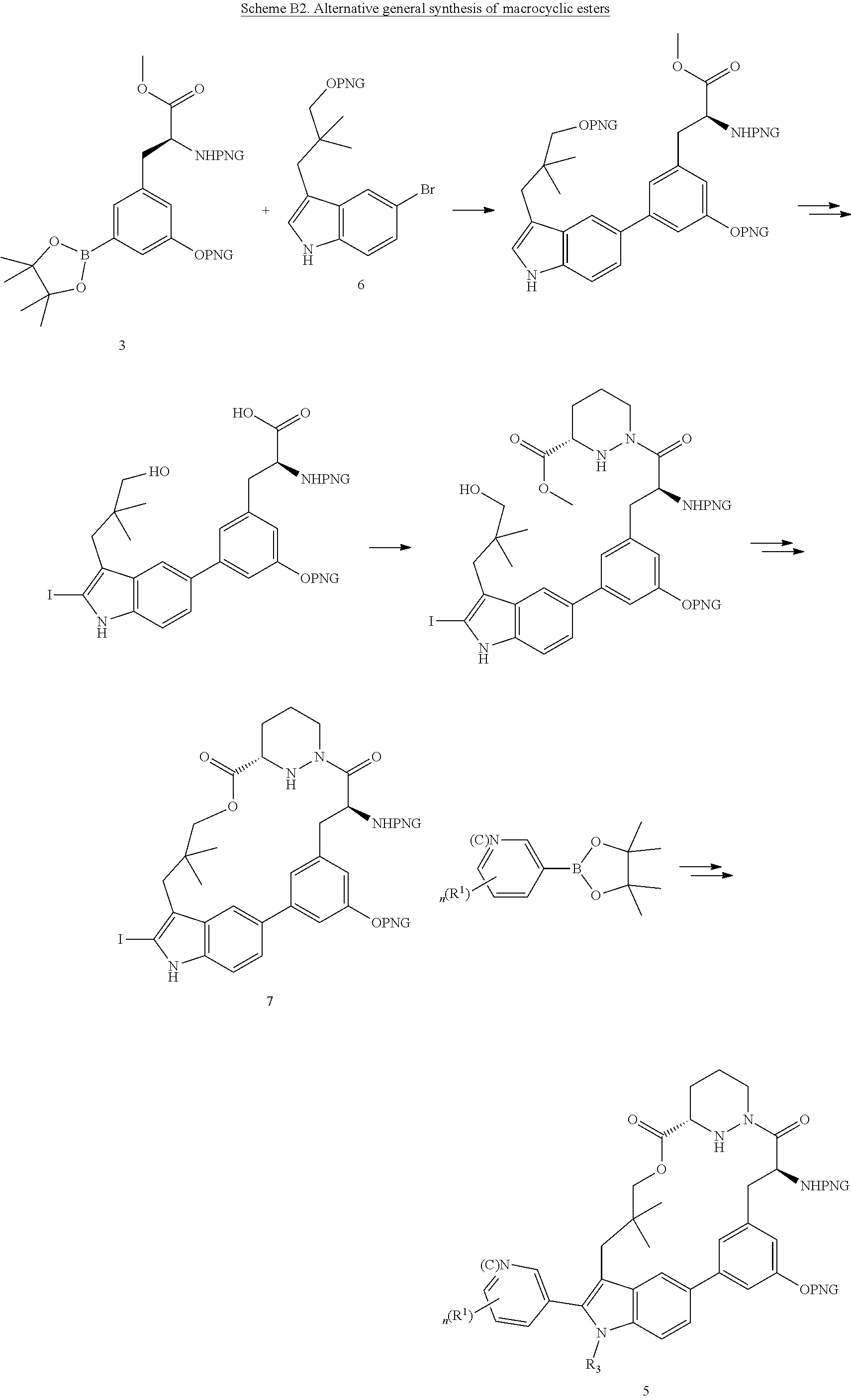

Alternatively, macrocyclic ester can be prepared as described in Scheme B2. An appropriately protected bromo-indolyl (6) coupled in the presence of a Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of a Pd catalyst with an appropriately substituted boronic ester and alkylation can yield fully protected macrocycle (5). Additional deprotection or functionalization steps are required to produce the final compound.

In addition, compounds of the disclosure can be synthesized using the methods described in the Examples below or as described in WO 2021/091982, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (BI), where B, L and W are defined herein, including by using methods exemplified in the Example section herein and in WO 2021/091982.

Compounds of Table B1 herein were prepared using methods disclosed herein or were prepared using methods disclosed herein combined with the knowledge of one of skill in the art. Compounds of Table B2 may be prepared using methods disclosed herein or may be prepared using methods disclosed herein combined with the knowledge of one of skill in the art.

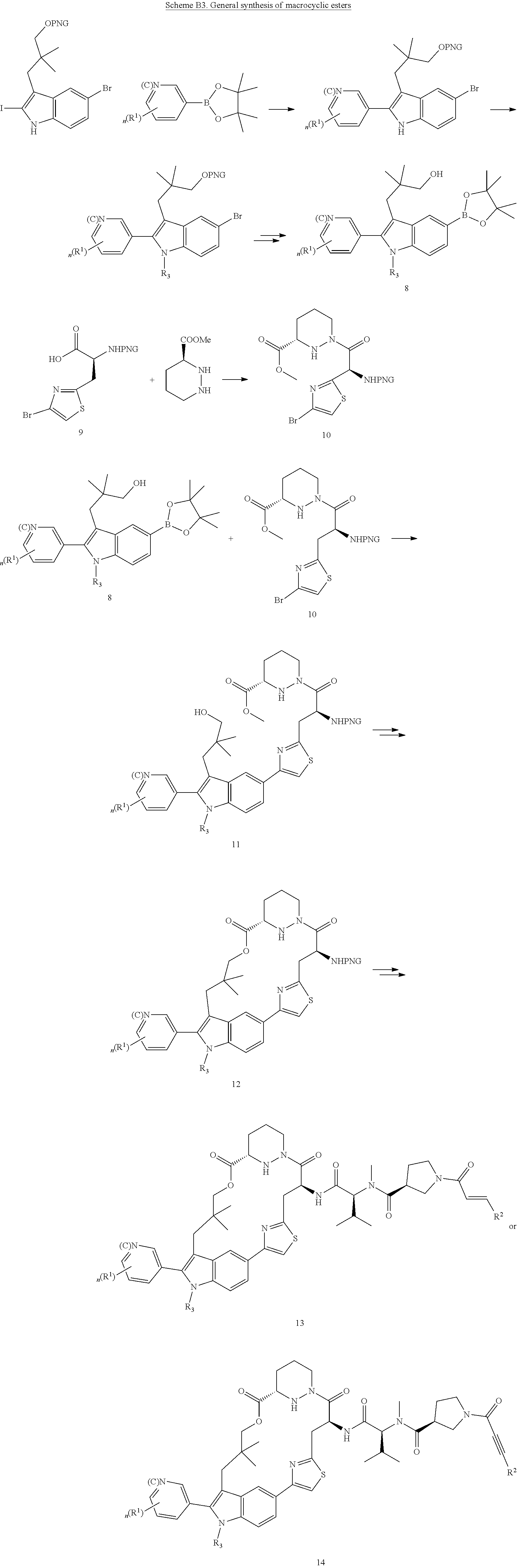

An alternative general synthesis of macrocyclic esters is outlined in Scheme B3. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted intermediate 4 can result in a macrocyclic product. Additional deprotection or functionalization steps could be required to produce a final compound 13 or 14.

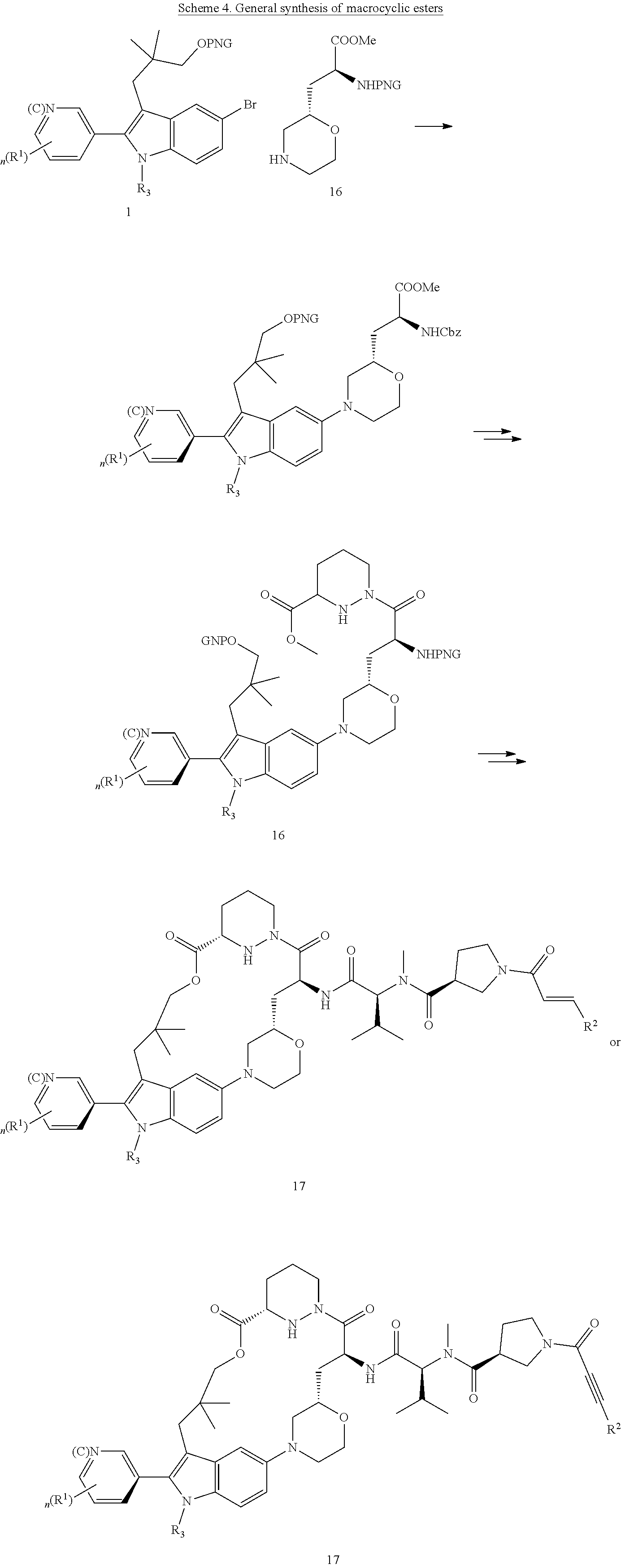

An alternative general synthesis of macrocyclic esters is outlined in Scheme B4. An appropriately substituted morpholine or an alternative heterocyclic intermediate (15) can be coupled with appropriately protected Intermediate 1 via Palladium mediated coupling. Subsequent ester hydrolysis, and coupling with piperazoic ester results in intermediate 16.

The macrocyclic esters can be made by hydrolysis, deprotection and macrocyclization sequence. Subsequent deprotection and coupling with Intermediate 4 (or analogs) result in an appropriately substituted final macrocyclic products. Additional deprotection or functionalization steps could be required to produce a final compound 17.

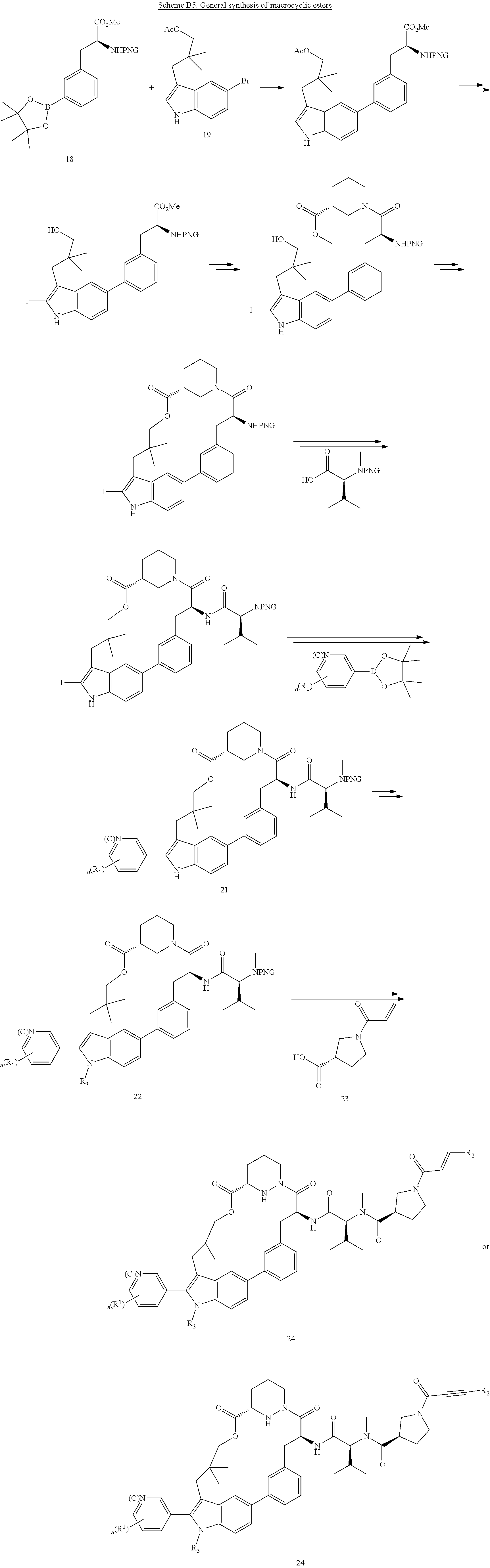

An alternative general synthesis of macrocyclic esters is outlined in Scheme B5. An appropriately substituted macrocycle (20) can be prepared starting from an appropriately protected boronic ester and bromo indolyl intermediate (19), including Palladium mediated coupling, hydrolysis, coupling with piperazoic ester, hydrolysis, de-protection, and macrocyclizarion steps. Subsequent coupling with an appropriately substituted protected amino acid followed by palladium mediated coupling yiels intermediate 21. Additional deprotection and derivatization steps, including alkylation may be required at this point.

The final macrocyclic esters can be made by coupling of intermediate (22) and an appropriately substituted carboxylic acid intermediate (23). Additional deprotection or functionalization steps could be required to produce a final compound (24).

In addition, compounds of the disclosure can be synthesized using the methods described in the Examples below and in WO 2021/091982, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (BI), where B, L and W are defined herein, including by using methods exemplified in the WO 2021/091982.

In some embodiments, the RAS(ON) inhibitor is a compound, or a pharmaceutically acceptable salt thereof, having the structure of Formula CI:

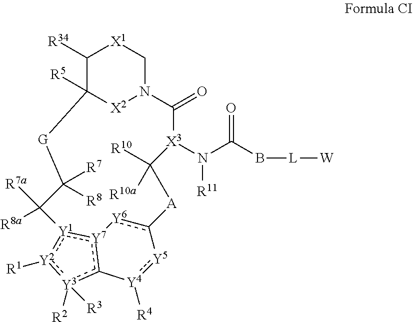

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl; and
R³⁴ is hydrogen or C₁-C₃ alkyl (e.g., methyl).

In some embodiments of Formula CI and subformula thereof, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula CI and subformula thereof, R³⁴ is hydrogen.

In some embodiments of Formula CI and subformula thereof, G is optionally substituted C₁-C₄ heteroalkylene.

In some embodiments, the RAS(ON) inhibitor has the structure of Formula CIa, or a pharmaceutically acceptable salt thereof:

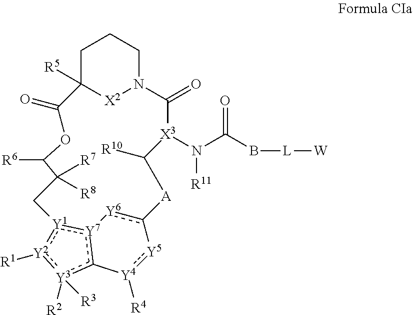

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula CI and subformula thereof, X² is NH. In some embodiments, X³ is CH.

In some embodiments of Formula CI and subformula thereof, R¹¹ is hydrogen. In some embodiments, R¹¹ is C₁-C₃ alkyl, such as methyl.

In some embodiments, the RAS(ON) inhibitor has the structure of Formula CIb, or a pharmaceutically acceptable salt thereof:

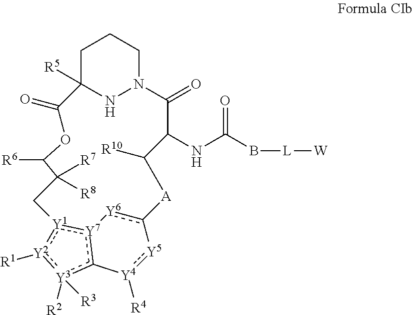

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula CI and subformula thereof, X¹ is optionally substituted C₁-C₂ alkylene. In some embodiments, X¹ is methylene.

In some embodiments of Formula CI and subformula thereof, R⁴ is hydrogen.

In some embodiments of Formula CI and subformula thereof, R⁵ is hydrogen. In some embodiments, R⁵ is C₁-C₄ alkyl optionally substituted with halogen. In some embodiments, R⁵ is methyl.

In some embodiments of Formula CI and subformula thereof, Y⁴ is C. In some embodiments of Formula CI and subformula thereof, R⁴ is hydrogen. In some embodiments of Formula CI and subformula thereof, Y⁵ is CH. In some embodiments of Formula CI and subformula thereof, Y⁶ is CH. In some embodiments of Formula CI and subformula thereof, Y¹ is C. In some embodiments of Formula CI and subformula thereof, Y² is C. In some embodiments of Formula CI and subformula thereof, Y³ is N. In some embodiments of Formula CI and subformula thereof, R³ is absent. In some embodiments of Formula CI and subformula thereof, Y⁷ is C.

In some embodiments, the RAS(ON) inhibitor has the structure of Formula CIc, or a pharmaceutically acceptable salt thereof:

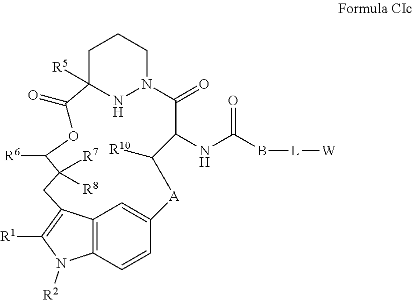

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷ is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula CI and subformula thereof, R⁶ is hydrogen.

In some embodiments of Formula CI and subformula thereof, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl, such as ethyl.

In some embodiments of Formula CI and subformula thereof, R⁷ is optionally substituted C₁-C₃ alkyl. In some embodiments, R⁷ is C₁-C₃ alkyl.

In some embodiments of Formula CI and subformula thereof, R⁸ is optionally substituted C₁-C₃ alkyl. In some embodiments, R⁸ is C₁-C₃ alkyl.

In some embodiments, the RAS(ON) inhibitor has the structure of Formula CId, or a pharmaceutically acceptable salt thereof:

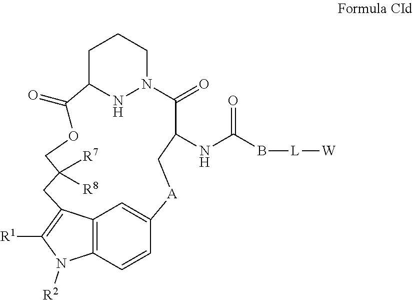

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula CI and subformula thereof, R¹ is 5 to 10-membered heteroaryl. In some embodiments, R¹ is optionally substituted 6-membered aryl or optionally substituted 6-membered heteroaryl.

In some embodiments, the RAS(ON) inhibitor has the structure of Formula Cle, or a pharmaceutically acceptable salt thereof:

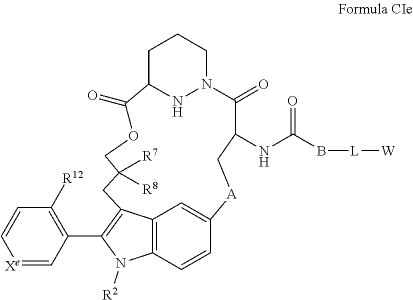

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl
X^{e} is N or CH; and
R¹² is optionally substituted C₁-C₆ alkyl or optionally substituted C₁-C₆ heteroalkyl.

In some embodiments of Formula CI and subformula thereof, X^{e} is N. In some embodiments, X^{e} is CH.

In some embodiments of Formula CI and subformula thereof, R¹² is optionally substituted C₁-C₆ heteroalkyl. In some embodiments, R¹² is

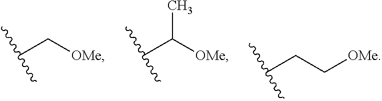

In some embodiments, R¹² is

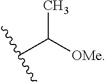 [SMILES: *C(C)OC]

In some embodiments, the RAS(ON) inhibitor has the structure of Formula CIf, or a pharmaceutically acceptable salt thereof:

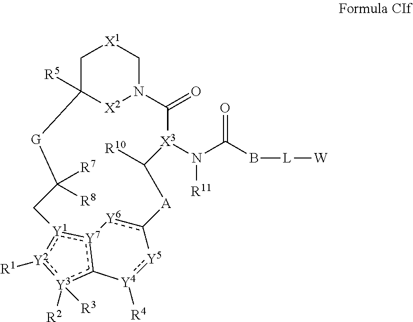

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, the RAS(ON) inhibitor has the structure of Formula CVI, or a pharmaceutically acceptable salt thereof:

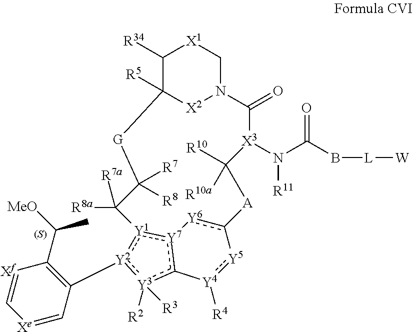

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N; Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R³⁴ is hydrogen or C₁-C₃ alkyl; and
X^{e} and X^{f} are, independently, N or CH.

In some embodiments, the RAS(ON) inhibitor has the structure of Formula CVIa, or a pharmaceutically acceptable salt thereof:

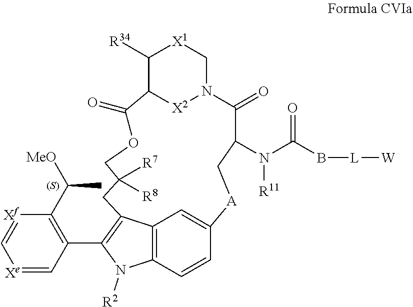

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of Formula CI and subformula thereof, X^{e} is N and X^{f} is CH. In some embodiments, X^{e} is CH and X^{f} is N.

In some embodiments, the RAS(ON) inhibitor has the structure of Formula CVIb, or a pharmaceutically acceptable salt thereof:

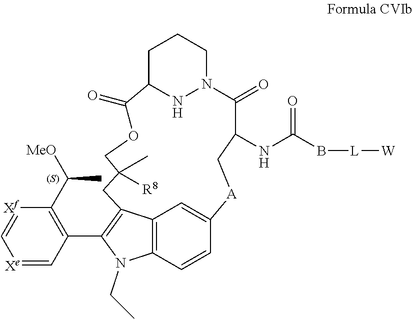

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
X^{e} and X^{f} are, independently, N or CH.

In some embodiments of Formula CI and subformula thereof, X^{e} is N and X^{f} is CH. In some embodiments, X^{e} is CH and X^{f} is N.

In some embodiments, the RAS(ON) inhibitor has the structure of Formula CVII, or a pharmaceutically acceptable salt thereof:

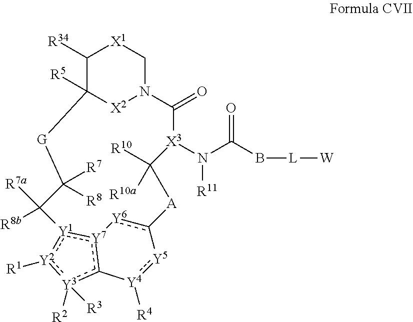

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is

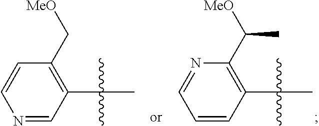 [SMILES: *c1cnccc1COC ; *c1cccnc1[C@H](C)OC]

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl (e.g., methyl).

In some embodiments of Formula CI and subformula thereof, A is optionally substituted 6-membered arylene. In some embodiments, A has the structure:

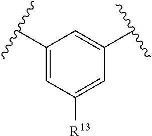

wherein R¹³ is hydrogen, hydroxy, amino, optionally substituted C₁-C₆ alkyl, or optionally substituted C₁-C₆ heteroalkyl. In some embodiments, R¹³ is hydrogen. In some embodiments, R¹³ is hydroxy.

In some embodiments of Formula C₁ and subformula thereof, B is —CHR⁹—. In some embodiments, R⁹ is optionally substituted C₁-C₆ alkyl or optionally substituted 3 to 6-membered cycloalkyl. In some embodiments, R⁹ is:

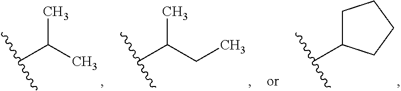

In some embodiments, R⁹ is:

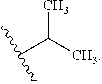 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula CI and subformula thereof, B is optionally substituted 6-membered arylene. In some embodiments, B is 6-membered arylene. In some embodiments, B is:

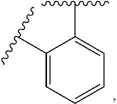 [SMILES: *c1ccccc1*]

In some embodiments of Formula CI and subformula thereof, R⁷ is methyl.

In some embodiments of Formula CI and subformula thereof, R⁸ is methyl.

In some embodiments of Formula CI and subformula thereof, R³⁴ is hydrogen.

In some embodiments of Formula CI and subformula thereof, the linker is the structure of Formula CII:

A¹-(B¹)_{f}—(C¹)_{g}—(B²)ₕ-(D¹)-(B³)ᵢ—(C²)ⱼ—(B⁴)ₖ-A² Formula CII
where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments of Formula CI and subformula thereof, the linker is acyclic. In some embodiments of Formula CI and subformula thereof, the linker has the structure of Formula CIIa:

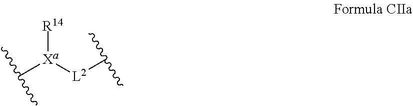

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

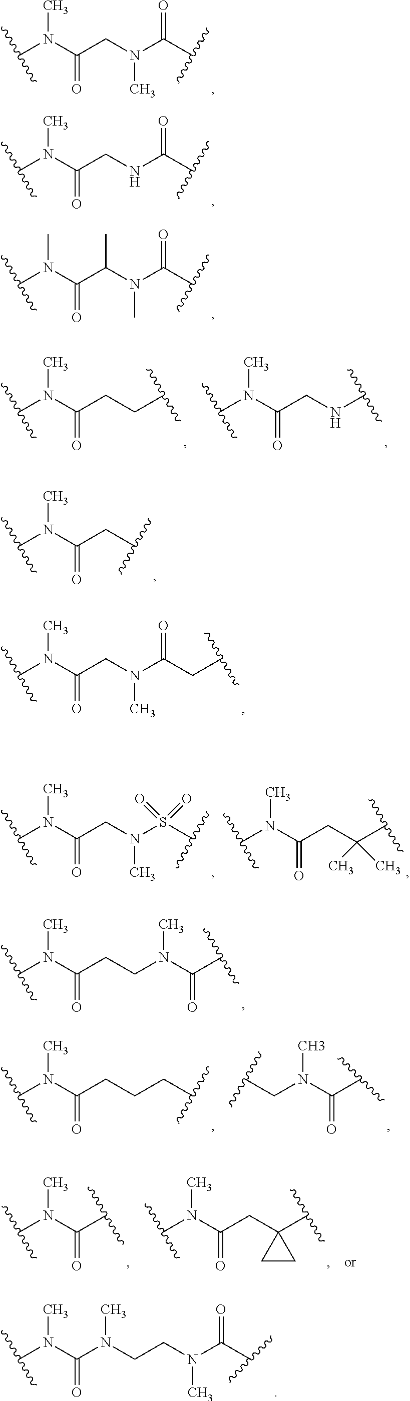

In some embodiments of Formula CI and subformula thereof, the linker is or a comprises a cyclic group. In some embodiments, the linker has the structure of Formula CIIb:

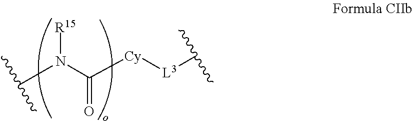

wherein o is 0 or 1;
R¹⁵ is hydrogen or optionally substituted C₁-C₆ alkyl; Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene. In some embodiments, the linker has the structure:

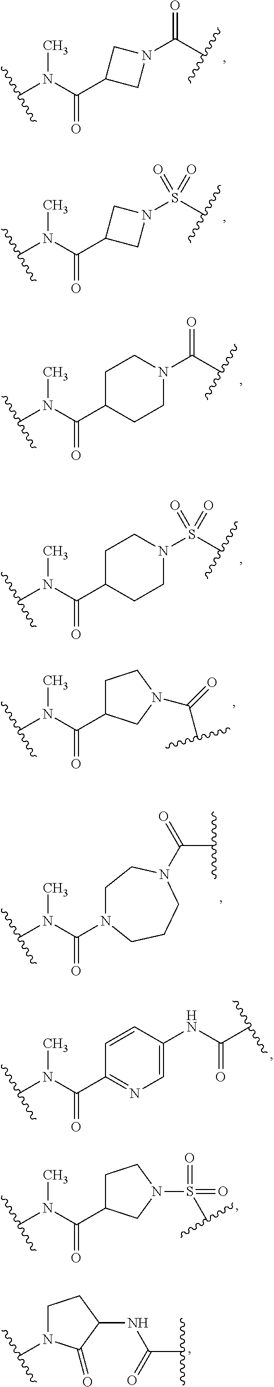

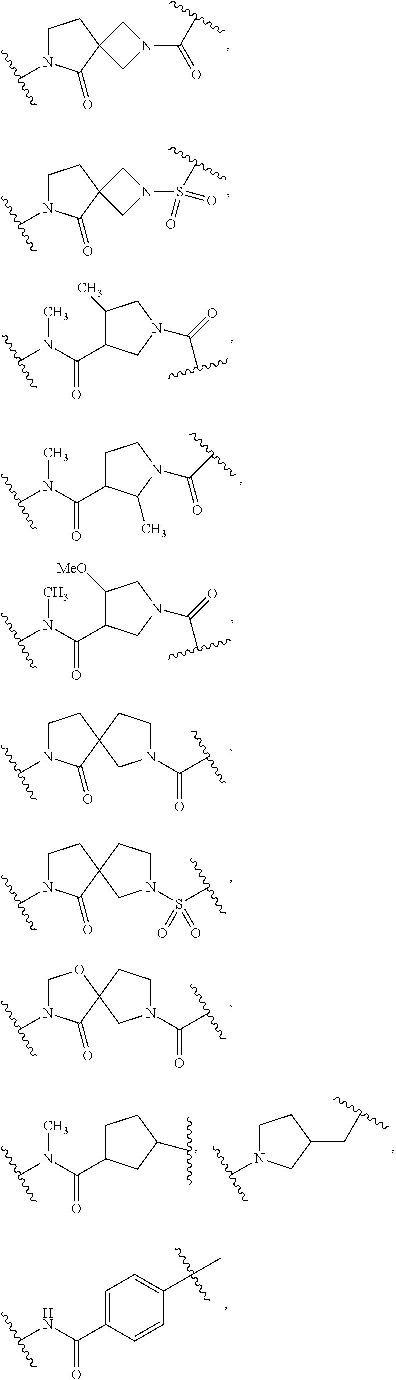

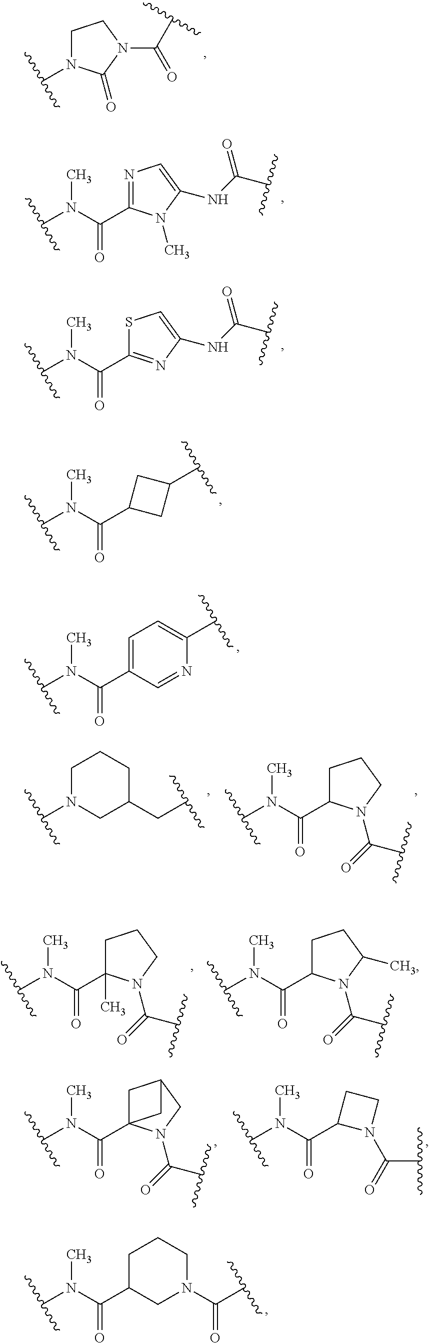

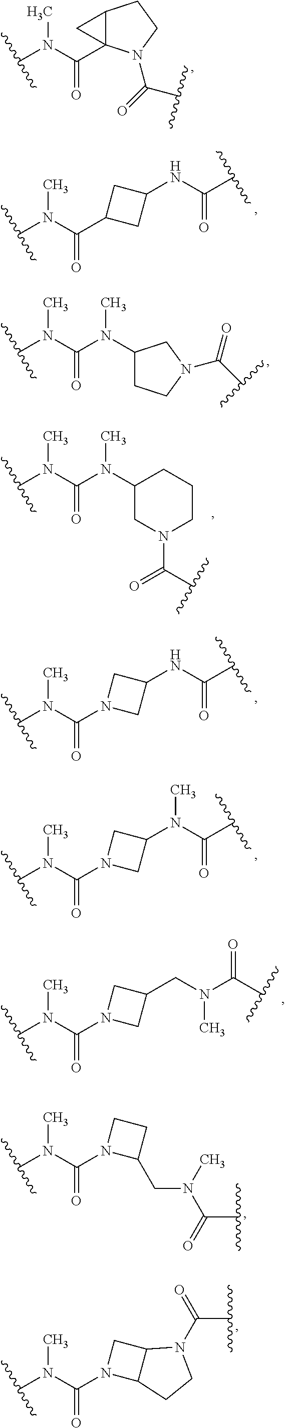

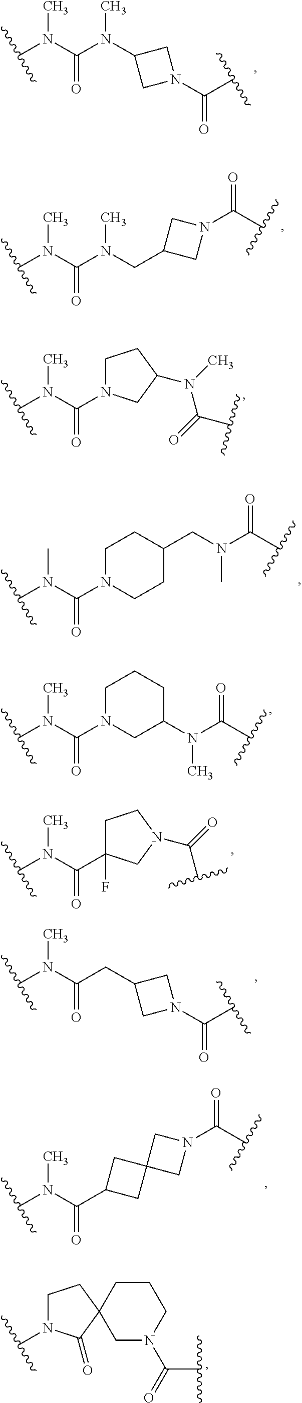

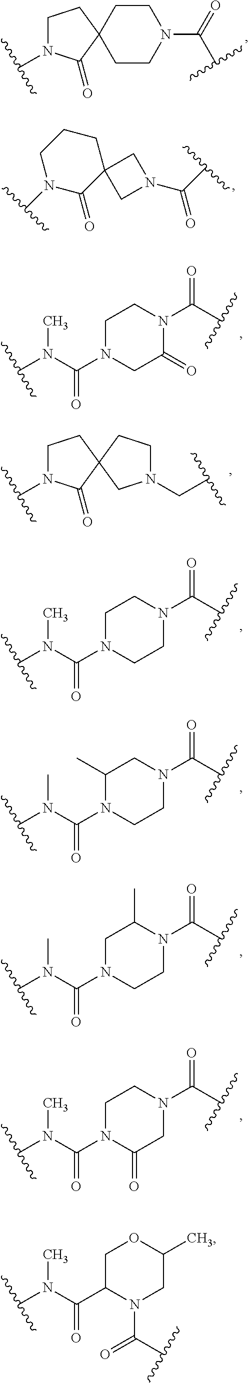

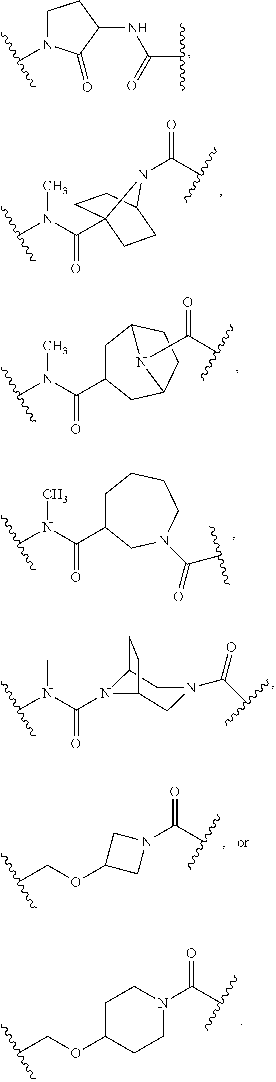

In some embodiments, a linker of Formula CII is selected from the group consisting of

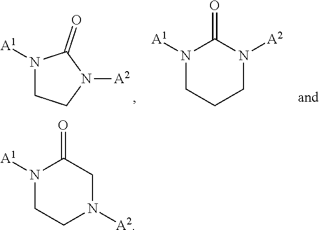

In some embodiments of Formula CI and subformula thereof, W comprises a carbodiimide. In some embodiments, W has the structure of Formula CIIIa:

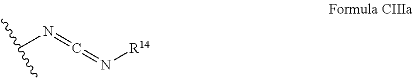

wherein R¹⁴ is optionally substituted C₁-C₆ alkyl, optionally substituted C₁-C₆ heteroalkyl, optionally substituted 3 to 8-membered cycloalkyl, optionally substituted 6 to 10-membered aryl, optionally substituted 3 to 14-membered heterocycloalkyl, or optionally substituted 5 to 10-membered heteroaryl. In some embodiments, W has the structure:

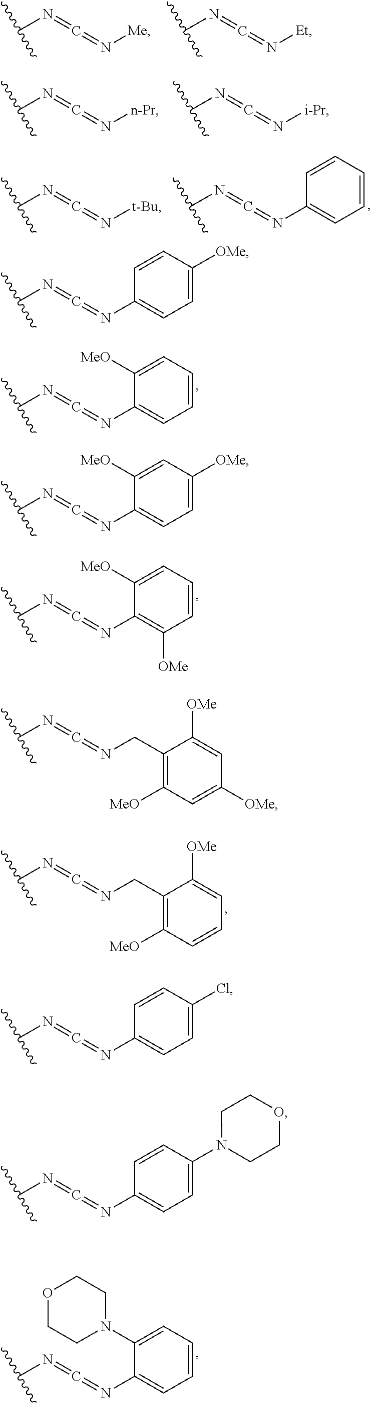

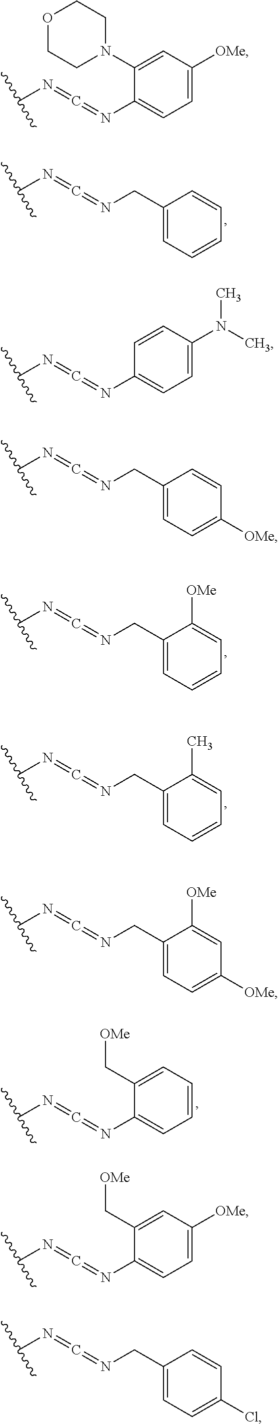

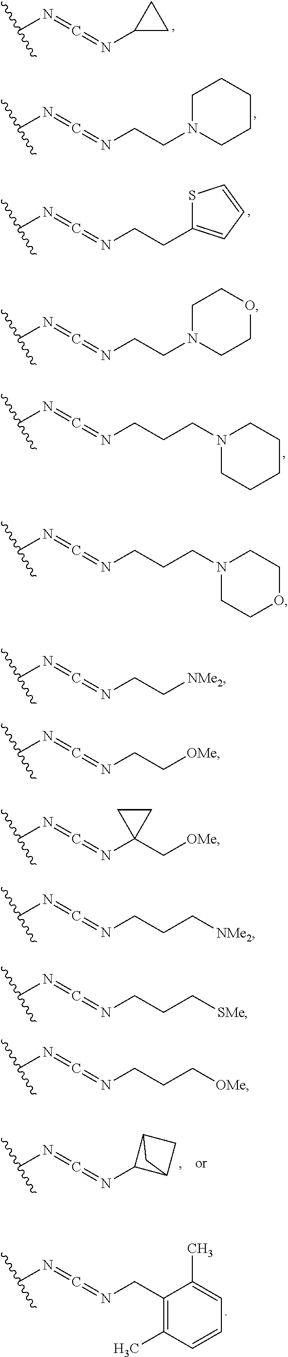

In some embodiments of Formula CI and subformula thereof, W comprises an oxazoline or thiazoline. In some embodiments, W has the structure of Formula CIIb:

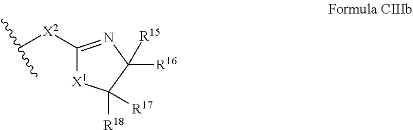

wherein X¹ is O or S;
X² is absent or NR¹⁹;
R¹⁵, R¹⁶, R¹⁷, and R¹⁸ are, independently, hydrogen or optionally substituted C₁-C₆ alkyl; and
R¹⁹ is hydrogen, C(O)(optionally substituted C₁-C₆ alkyl), optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

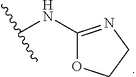 [SMILES: *NC1=NCCO1]

In some embodiments of Formula CI and subformula thereof, W comprises a chloroethyl urea, a chloroethyl thiourea, a chloroethyl carbamate, or a chloroethyl thiocarbamate. In some embodiments, W has the structure of Formula CIIIc:

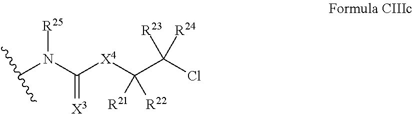

wherein X³ is O or S;
X⁴ is O, S, NR²⁶;
R²¹, R²², R²³, R²⁴, and R²⁶ are, independently, hydrogen or optionally substituted C₁-C₆ alkyl; and
R²⁵ is hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

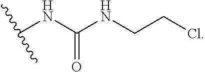 [SMILES: *NC(=O)NCCCl]

In some embodiments of Formula CI and subformula thereof, W comprises an aziridine. In some embodiments, W has the structure of Formula CIIId1, Formula CIIId2, Formula CIIId3, or Formula CIIId4:

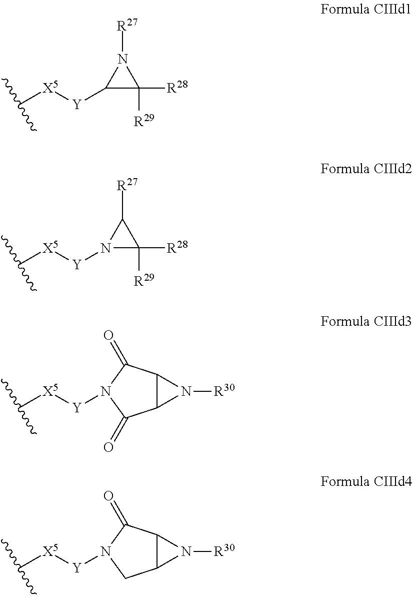

wherein X⁵ is absent or NR³⁰;
Y is absent or C(O), C(S), S(O), SO₂, or optionally substituted C₁-C₃ alkylene;
R²⁷ is hydrogen, —C(O)R³², —C(O)OR³², —SOR³³, —SO₂R³³, optionally substituted C₁-C₆ alkyl, optionally substituted 6 to 10-membered aryl, optionally substituted 3 to 14-membered heterocycloalkyl, or optionally substituted 5 to 10-membered heteroaryl;
R²⁸ and R²⁹ are, independently, hydrogen, CN, C(O)R³¹, CO₂R³¹, C(O)R³¹R³¹ optionally substituted C₁-C₆ alkyl, optionally substituted 3 to 10-membered cycloalkyl, optionally substituted 6 to 10-membered aryl, optionally substituted 3 to 14-membered heterocycloalkyl, or optionally substituted 5 to 10-membered heteroaryl;
each R³¹ is, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl;
R³⁰ is hydrogen or optionally substituted C₁-C₆ alkyl; and
R³² and R³³ are, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is:

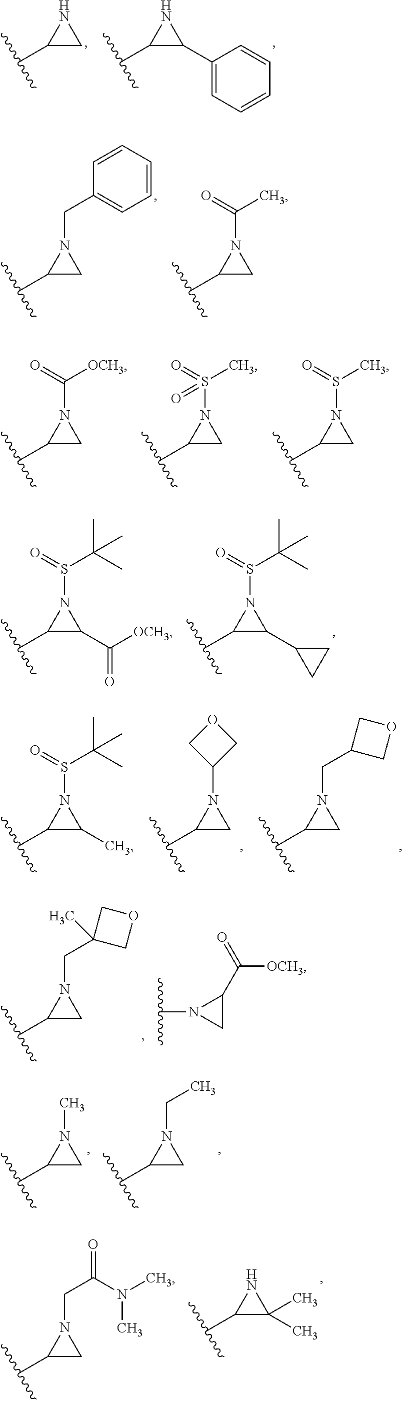

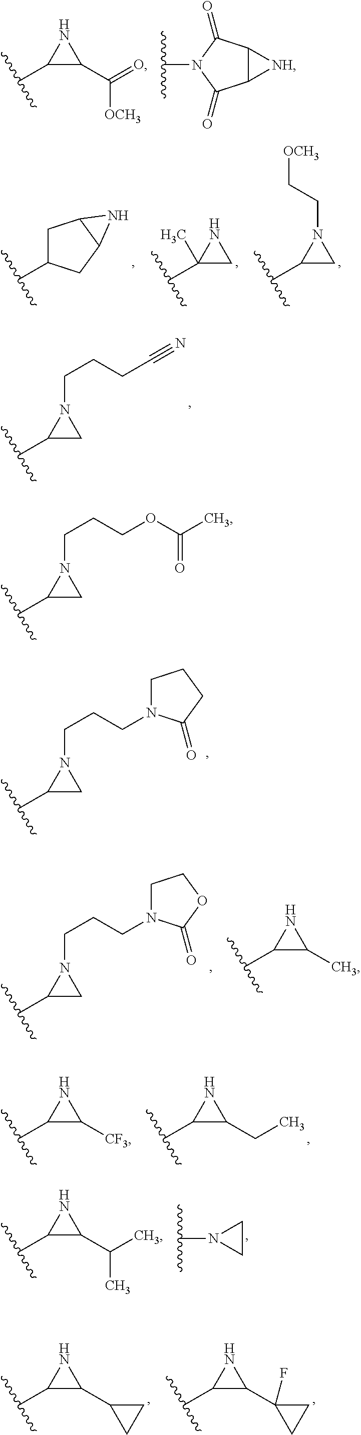

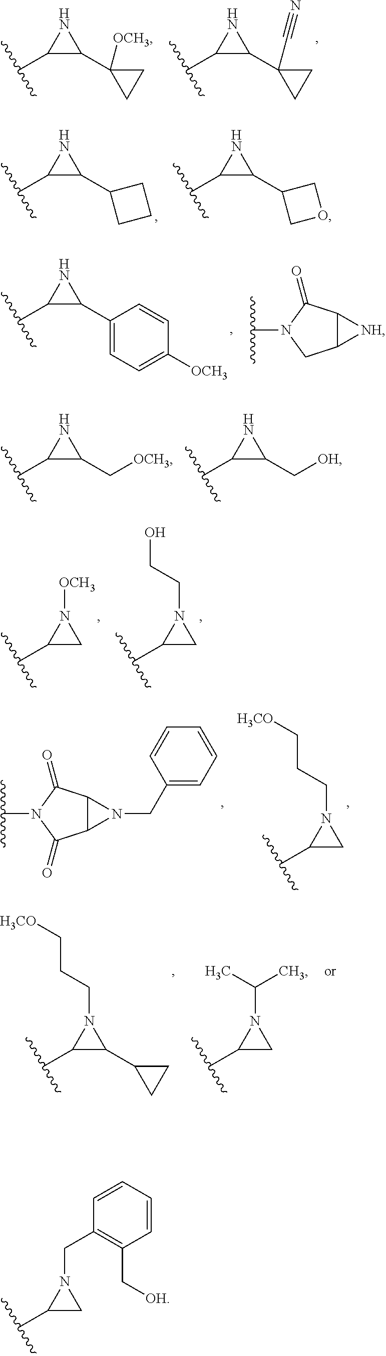

In some embodiments of Formula CI and subformula thereof, W comprises an epoxide. In some embodiments, W is

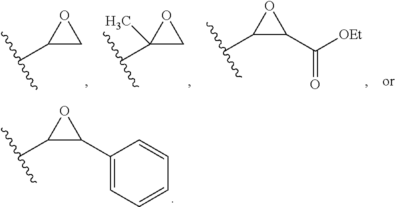

In some embodiments of Formula CI and subformula thereof, W is a cross-linking group bound to an organic moiety that is a Ras binding moiety, i.e., RBM-W, wherein upon contact of an RBM-W compound with a Ras protein, the RBM-W binds to the Ras protein to form a conjugate. For example, the W moiety of an RBM-W compound may bind, e.g., cross-link, with an amino acid of the Ras protein to form the conjugate. In some embodiments, the Ras binding moiety is a K-Ras binding moiety. In some embodiments, the K-Ras binding moiety binds to a residue of a K-Ras Switch-II binding pocket of the K-Ras protein. In some embodiments, the Ras binding moiety is an H-Ras binding moiety that binds to a residue of an H-Ras Switch-II binding pocket of an H-Ras protein. In some embodiments, the Ras binding moiety is an N-Ras binding moiety that binds to a residue of an N-Ras Switch-II binding pocket of an N-Ras protein. The W of an RBM-W compound may comprise any W described herein. The Ras binding moiety typically has a molecular weight of under 1200 Da. See, e.g., see, e.g., Johnson et al., 292:12981-12993 (2017) for a description of Ras protein domains, incorporated herein by reference.

In some embodiments, the RAS(ON) inhibitor is selected from Table C1, or a pharmaceutically acceptable salt or stereoisomer thereof. In some embodiments, the RAS(ON) inhibitor is selected from Table C1, or a pharmaceutically acceptable salt or atropisomer thereof.

TABLE C1
Certain Compounds of the Present Invention

| Ex# | Structure |
|---|---|
| C1 | 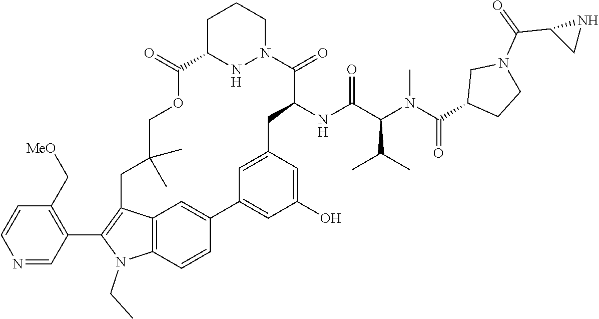 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@H]3CN3)C1)C(=O)N1CCC[C@H](N1)C(=O)OCC(C)(C)C2] |
| C2 | 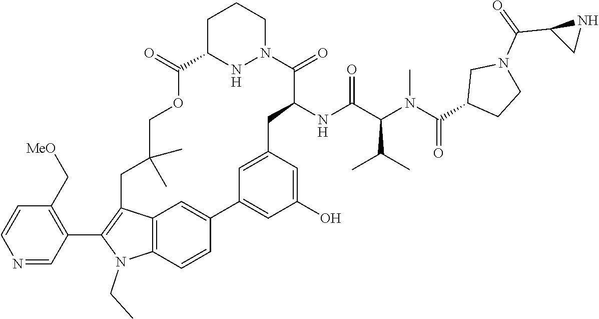 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@@H]3CN3)C1)C(=O)N1CCC[C@H](N1)C(=O)OCC(C)(C)C2] |
| C3 | 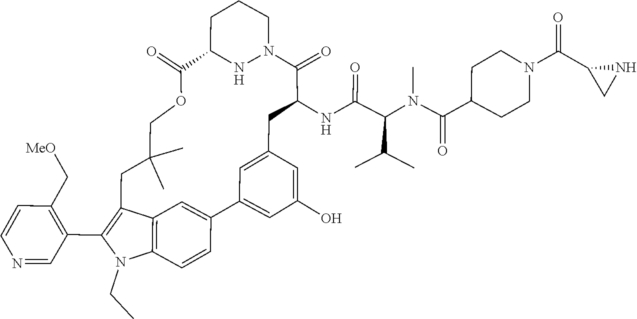 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@H]3CN3)CC1)C(=O)N1CCC[C@H](N1)C(=O)OCC(C)(C)C2] |
| C4 | 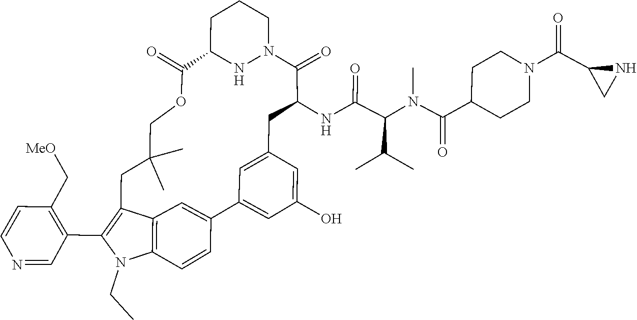 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@@H]3CN3)CC1)C(=O)N1CCC[C@H](N1)C(=O)OCC(C)(C)C2] |
| C5 | 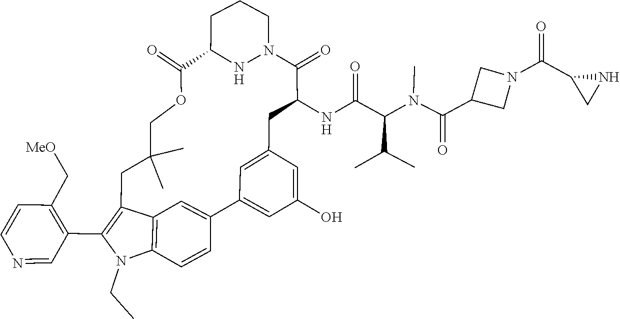 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@H]3CN3)C1)C(=O)N1CCC[C@H](N1)C(=O)OCC(C)(C)C2] |
| C6 | 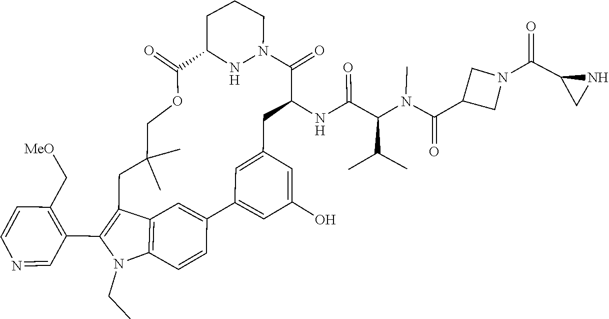 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@@H]3CN3)C1)C(=O)N1CCC[C@H](N1)C(=O)OCC(C)(C)C2] |
| C7 | 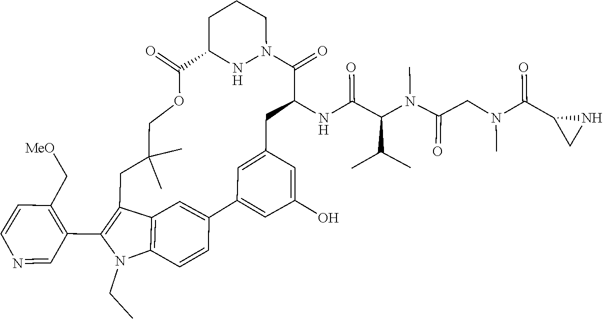 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1)C(=O)N1CCC[C@H](N1)C(=O)OCC(C)(C)C2] |
| C8 | 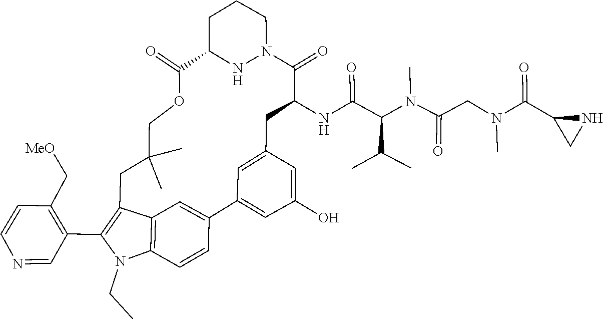 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1)C(=O)N1CCC[C@H](N1)C(=O)OCC(C)(C)C2] |
| C9 | 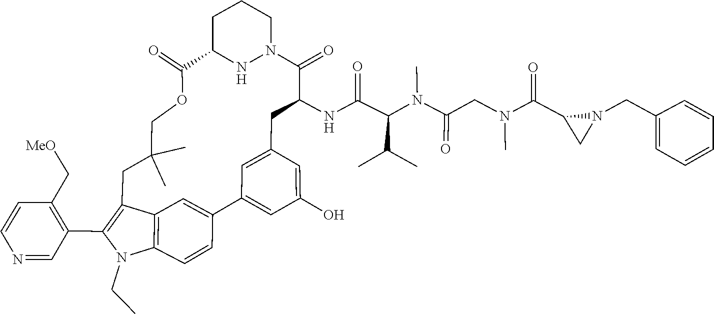 |
| C10 | 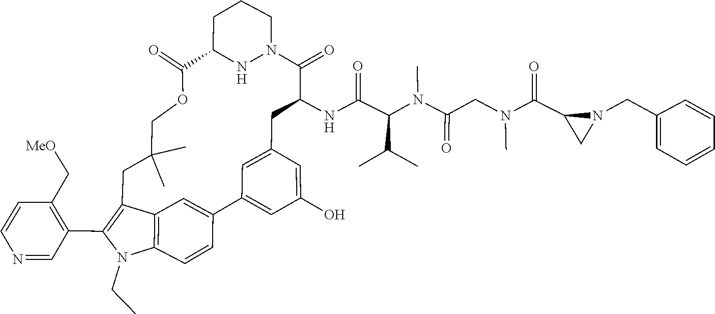 |
| C11 | 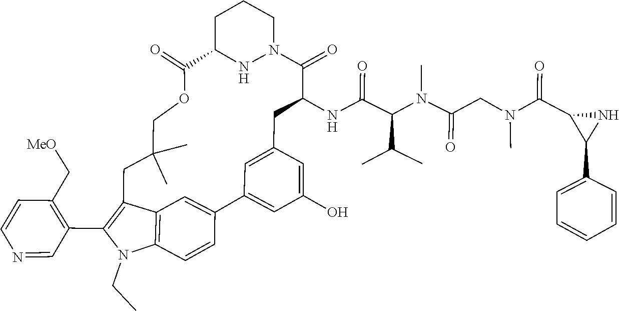 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1c1ccccc1)C(=O)N1CCC[C@H](N1)C(=O)OCC(C)(C)C2] |
| C12 | 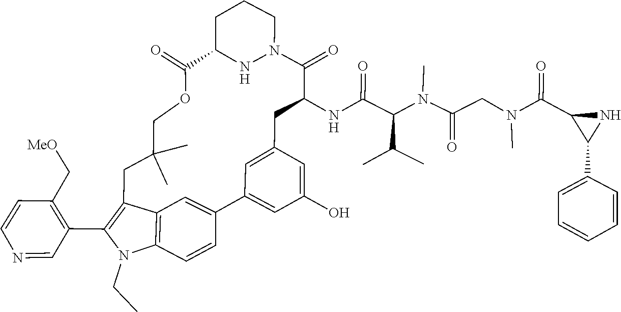 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1c1ccccc1)C(=O)N1CCC[C@H](N1)C(=O)OCC(C)(C)C2] |
| C13 | 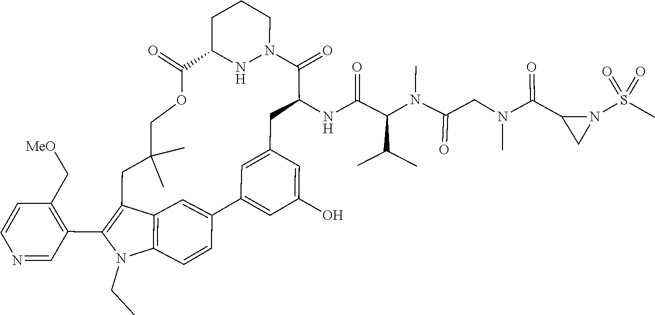 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1S(C)(=O)=O)C(=O)N1CCC[C@H](N1)C(=O)OCC(C)(C)C2] |
| C14 | 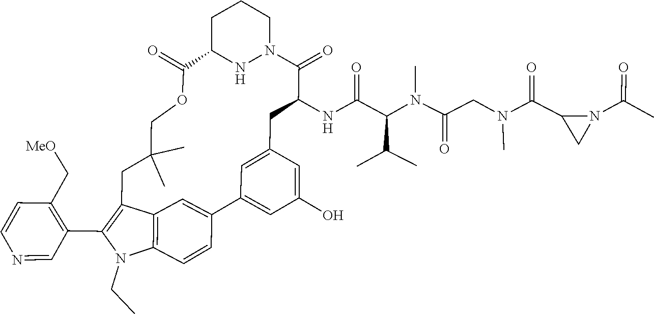 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1C(C)=O)C(=O)N1CCC[C@H](N1)C(=O)OCC(C)(C)C2] |
| C15 A and B | 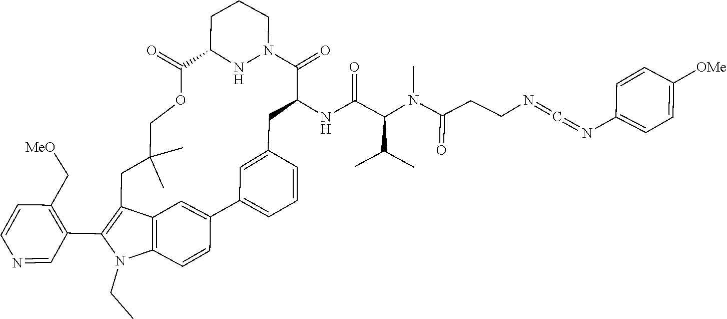 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1)C(=O)N1CCC[C@H](N1)C(=O)OCC(C)(C)C2] |
| C16 A and B | 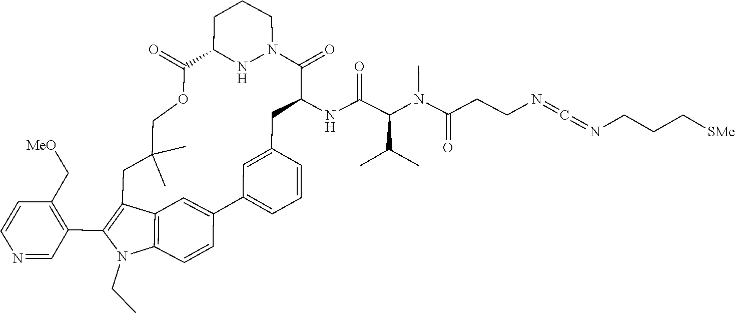 [SMILES: CCn1c(-c2cnccc2COC)c2c3cc(ccc31)-c1cccc(c1)C[C@H](NC(=O)[C@H](C(C)C)N(C)C(=O)CCN=C=NCCCSC)C(=O)N1CCC[C@H](N1)C(=O)OCC(C)(C)C2] |
| C17 A and B | 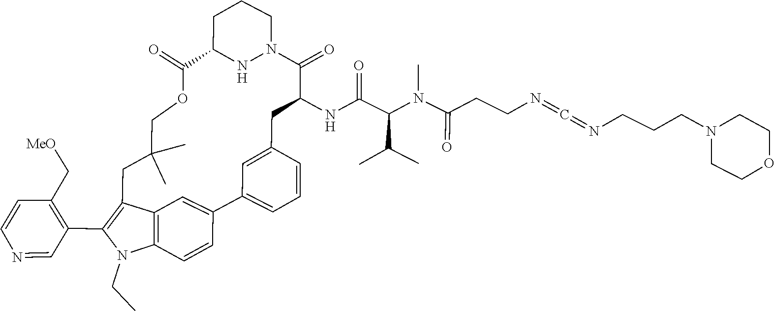 [SMILES: CCn1c(-c2cnccc2COC)c2c3cc(ccc31)-c1cccc(c1)C[C@H](NC(=O)[C@H](C(C)C)N(C)C(=O)CCN=C=NCCCN1CCOCC1)C(=O)N1CCC[C@H](N1)C(=O)OCC(C)(C)C2] |
| C18 A and B | 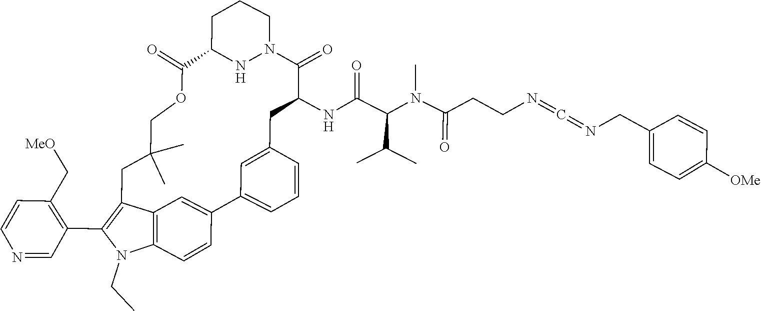 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccc(OC)cc1)C(=O)N1CCC[C@H](N1)C(=O)OCC(C)(C)C2] |
| C19 A and B | 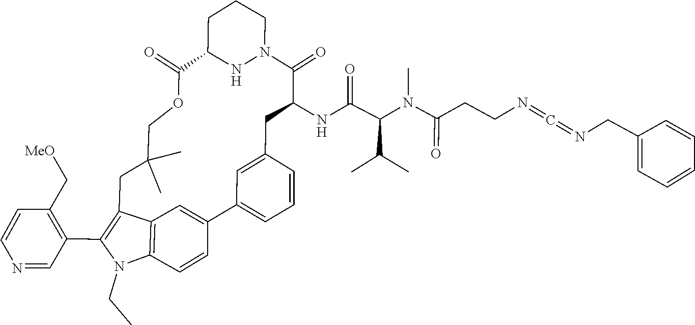 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccccc1)C(=O)N1CCC[C@H](N1)C(=O)OCC(C)(C)C2] |
| C20 A and B | 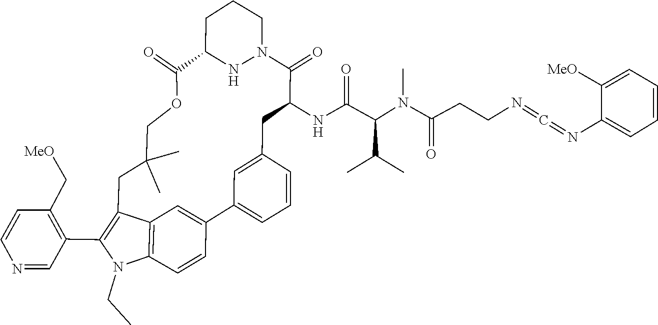 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1OC)C(=O)N1CCC[C@H](N1)C(=O)OCC(C)(C)C2] |
| C21 A and B | 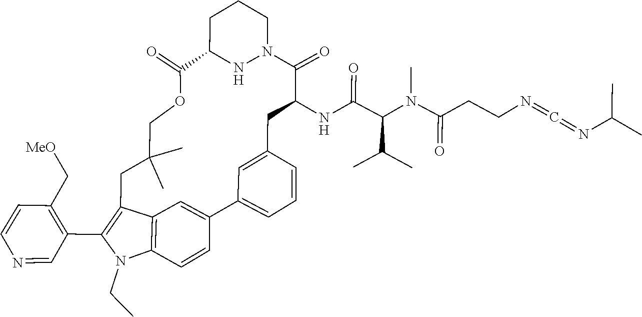 [SMILES: CCn1c(-c2cnccc2COC)c2c3cc(ccc31)-c1cccc(c1)C[C@H](NC(=O)[C@H](C(C)C)N(C)C(=O)CCN=C=NC(C)C)C(=O)N1CCC[C@H](N1)C(=O)OCC(C)(C)C2] |
| C22 A and B | 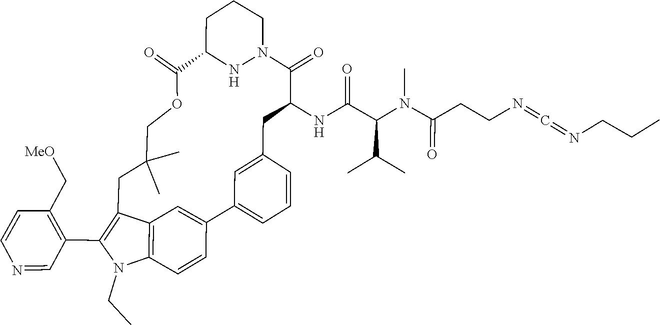 [SMILES: CCCN=C=NCCC(=O)N(C)[C@H](C(=O)N[C@H]1Cc2cccc(c2)-c2ccc3c(c2)c(c(-c2cnccc2COC)n3CC)CC(C)(C)COC(=O)[C@@H]2CCCN(N2)C1=O)C(C)C] |
| C23 A and B | 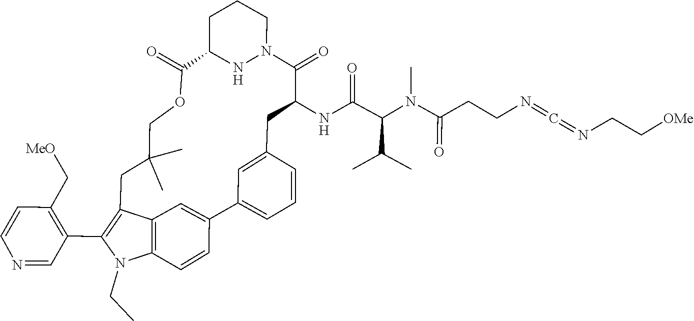 [SMILES: CCn1c(-c2cnccc2COC)c2c3cc(ccc31)-c1cccc(c1)C[C@H](NC(=O)[C@H](C(C)C)N(C)C(=O)CCN=C=NCCOC)C(=O)N1CCC[C@H](N1)C(=O)OCC(C)(C)C2] |
| C24 A and B | 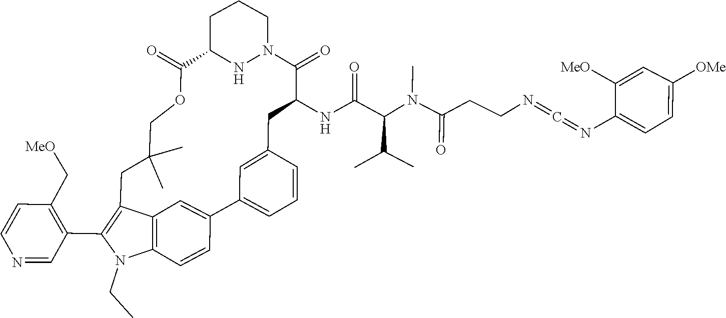 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1OC)C(=O)N1CCC[C@H](N1)C(=O)OCC(C)(C)C2] |
| C25 | 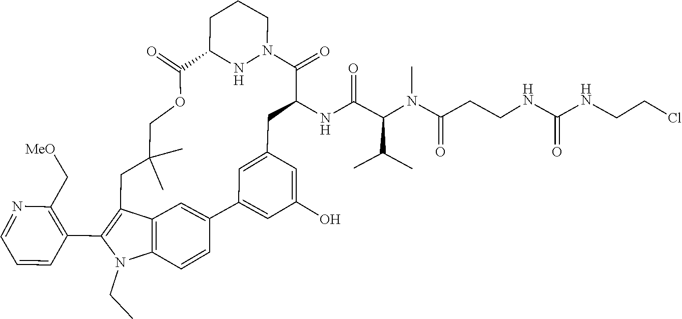 [SMILES: CCn1c(-c2cccnc2COC)c2c3cc(ccc31)-c1cc(O)cc(c1)C[C@H](NC(=O)[C@H](C(C)C)N(C)C(=O)CCNC(=O)NCCCl)C(=O)N1CCC[C@H](N1)C(=O)OCC(C)(C)C2] |
| C26 | 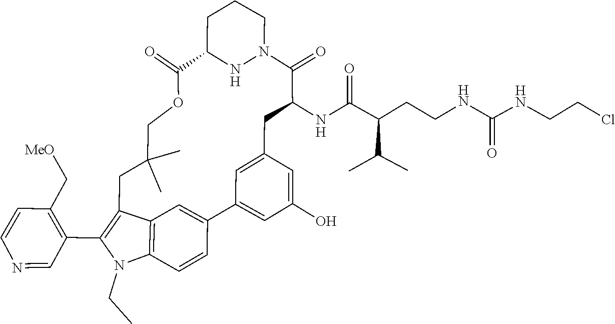 [SMILES: CCn1c(-c2cnccc2COC)c2c3cc(ccc31)-c1cc(O)cc(c1)C[C@H](NC(=O)[C@@H](CCNC(=O)NCCCl)C(C)C)C(=O)N1CCC[C@H](N1)C(=O)OCC(C)(C)C2] |
| C27 | 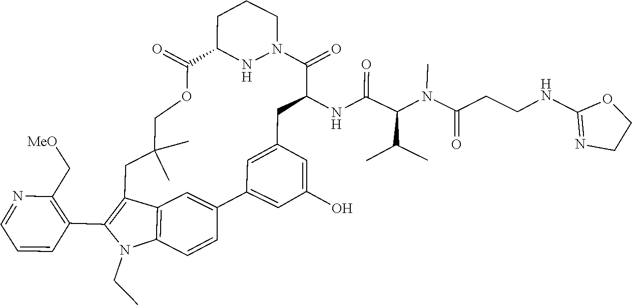 [SMILES: CCn1c(-c2cccnc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| C28 | 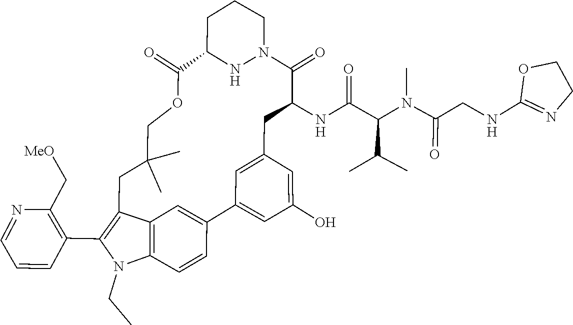 [SMILES: CCn1c(-c2cccnc2COC)c2c3cc(ccc31)-c1cc(O)cc(c1)C[C@H](NC(=O)[C@H](C(C)C)N(C)C(=O)CNC1=NCCO1)C(=O)N1CCC[C@H](N1)C(=O)OCC(C)(C)C2] |
| C29 | 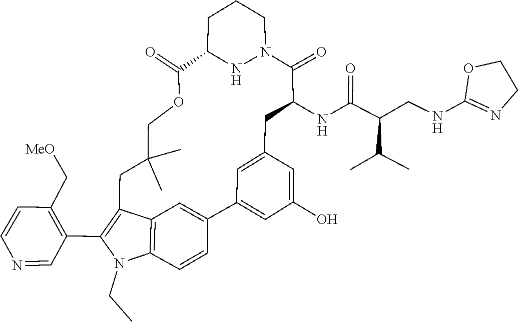 [SMILES: CCn1c(-c2cnccc2COC)c2c3cc(ccc31)-c1cc(O)cc(c1)C[C@H](NC(=O)[C@@H](CNC1=NCCO1)C(C)C)C(=O)N1CCC[C@H](N1)C(=O)OCC(C)(C)C2] |
| C30 | 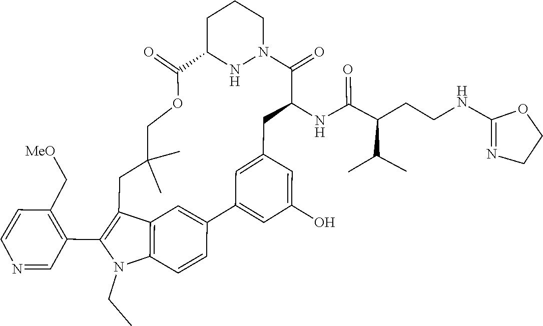 [SMILES: CCn1c(-c2cnccc2COC)c2c3cc(ccc31)-c1cc(O)cc(c1)C[C@H](NC(=O)[C@@H](CCNC1=NCCO1)C(C)C)C(=O)N1CCC[C@H](N1)C(=O)OCC(C)(C)C2] |
| C31 | 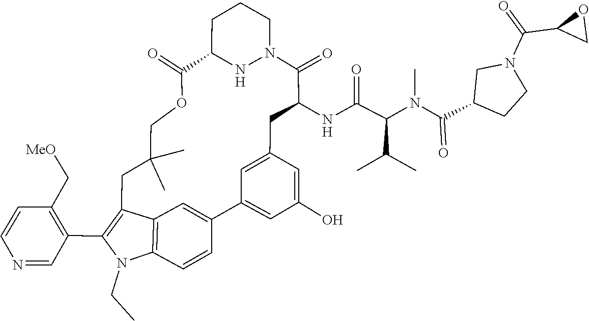 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@@H]3CO3)C1)C(=O)N1CCC[C@H](N1)C(=O)OCC(C)(C)C2] |
| C32 A and B | 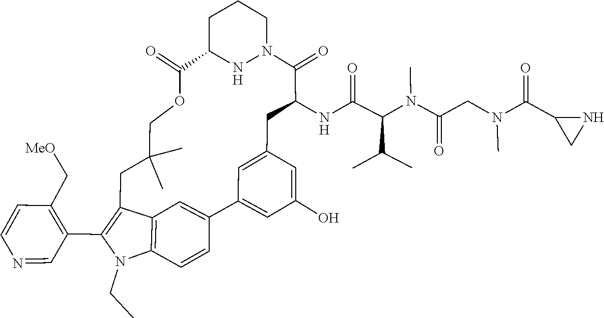 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| C33 | 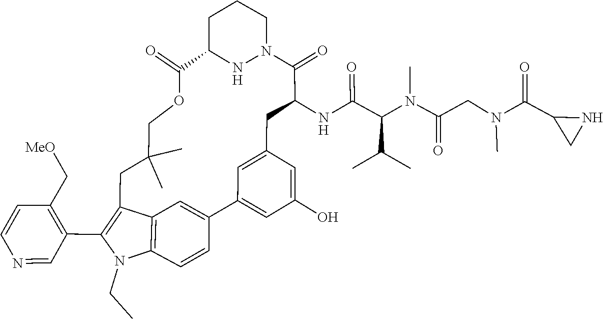 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| C34 | 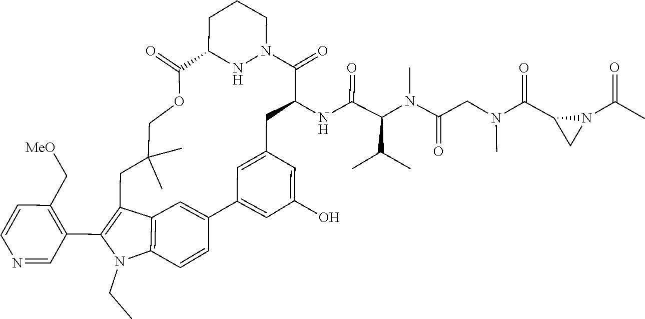 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(C)=O)C(=O)N1CCC[C@H](N1)C(=O)OCC(C)(C)C2] |
| C35 | 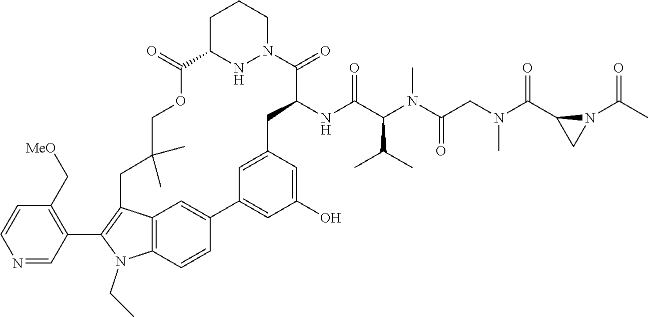 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(C)=O)C(=O)N1CCC[C@H](N1)C(=O)OCC(C)(C)C2] |
| C36 | 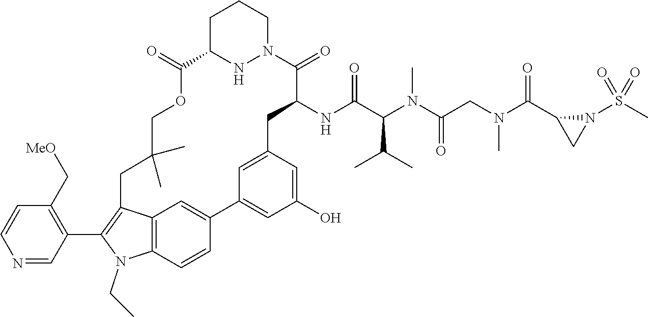 |
| C37 | 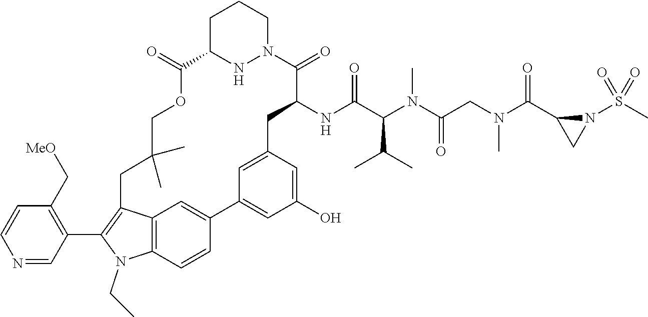 |
| C38 | 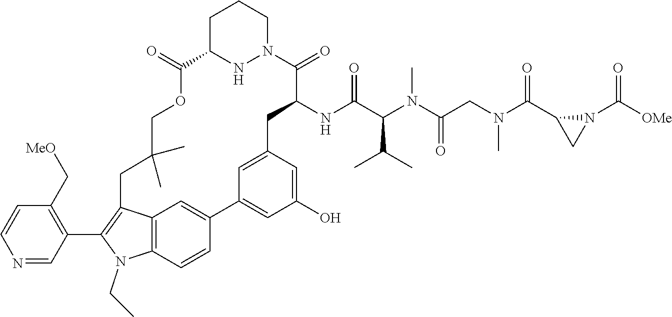 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(=O)OC)C(=O)N1CCC[C@H](N1)C(=O)OCC(C)(C)C2] |
| C39 | 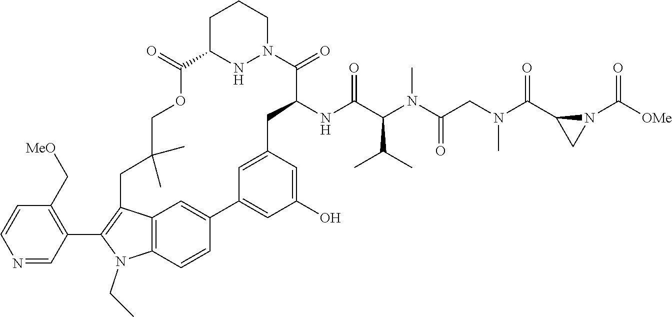 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(=O)OC)C(=O)N1CCC[C@H](N1)C(=O)OCC(C)(C)C2] |
| C40 | 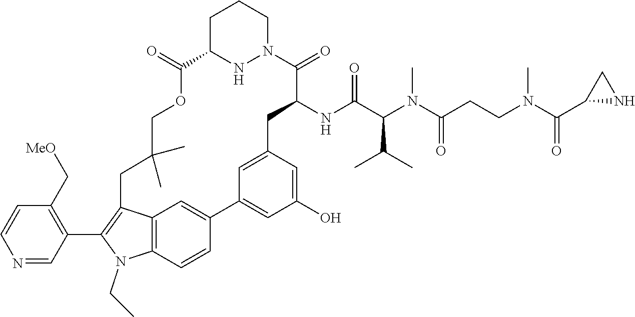 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@@H]1CN1)C(=O)N1CCC[C@H](N1)C(=O)OCC(C)(C)C2] |
| C41 | 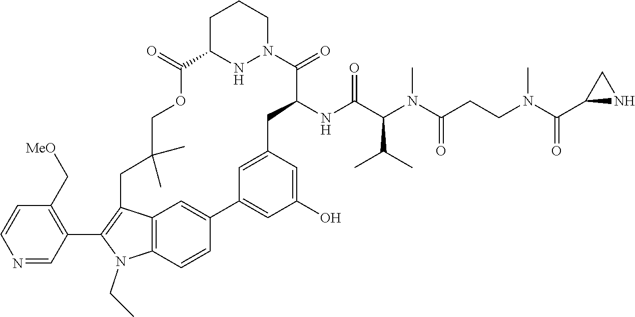 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@H]1CN1)C(=O)N1CCC[C@H](N1)C(=O)OCC(C)(C)C2] |
| C42A | 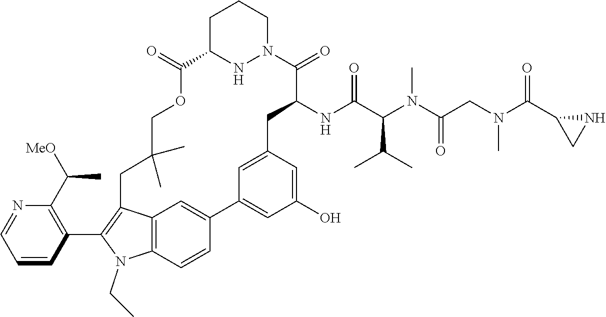 |
| C42B | 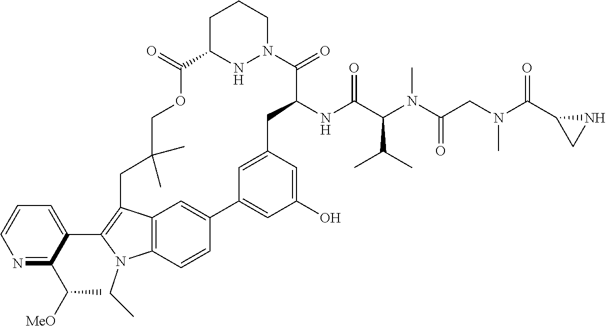 |
| C43A | 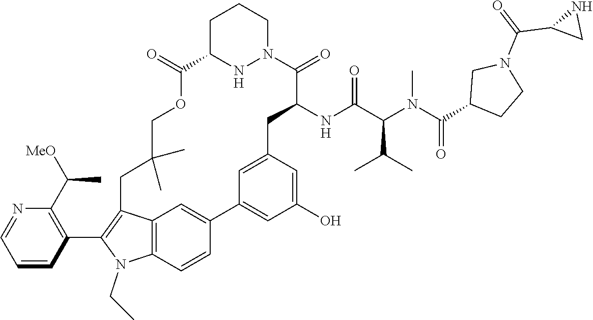 |
| C43B | 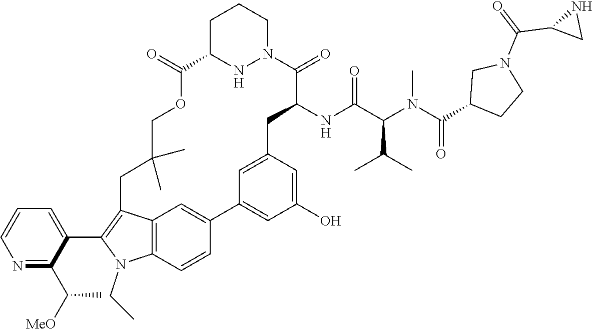 |
| C44 | 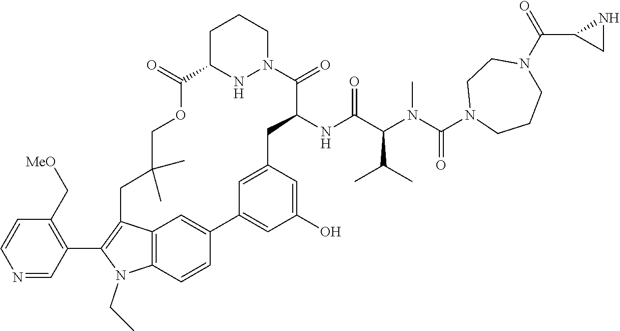 [SMILES: CCn1c(-c2cnccc2COC)c2c3cc(ccc31)-c1cc(O)cc(c1)C[C@H](NC(=O)[C@H](C(C)C)N(C)C(=O)N1CCCN(C(=O)[C@H]3CN3)CC1)C(=O)N1CCC[C@H](N1)C(=O)OCC(C)(C)C2] |
| C45 | 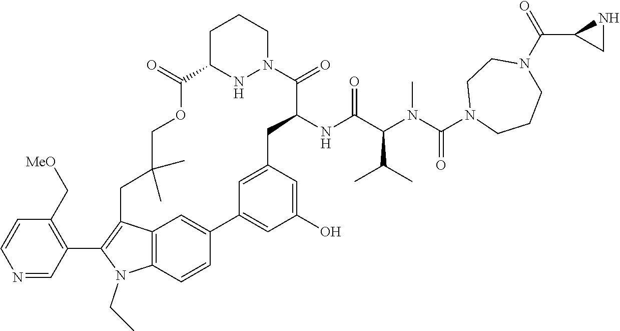 [SMILES: CCn1c(-c2cnccc2COC)c2c3cc(ccc31)-c1cc(O)cc(c1)C[C@H](NC(=O)[C@H](C(C)C)N(C)C(=O)N1CCCN(C(=O)[C@@H]3CN3)CC1)C(=O)N1CCC[C@H](N1)C(=O)OCC(C)(C)C2] |
| C46 | 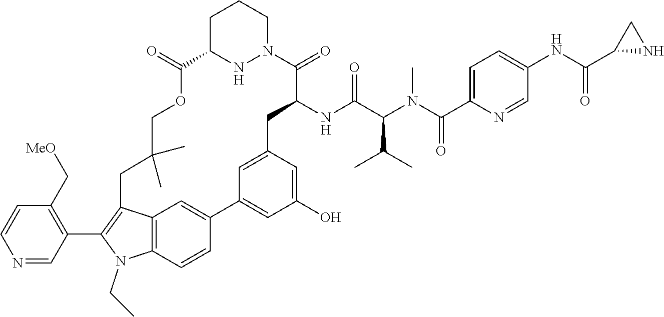 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3CN3)cn1)C(=O)N1CCC[C@H](N1)C(=O)OCC(C)(C)C2] |
| C47 A and B | 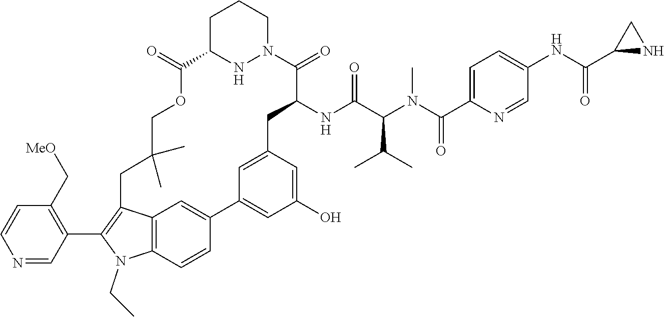 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3CN3)cn1)C(=O)N1CCC[C@H](N1)C(=O)OCC(C)(C)C2] |
| C48 | 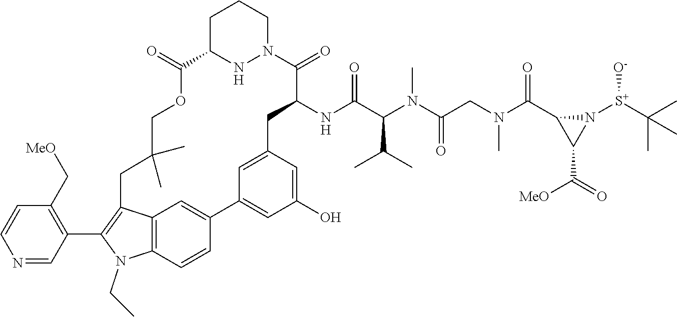 |
| C49 | 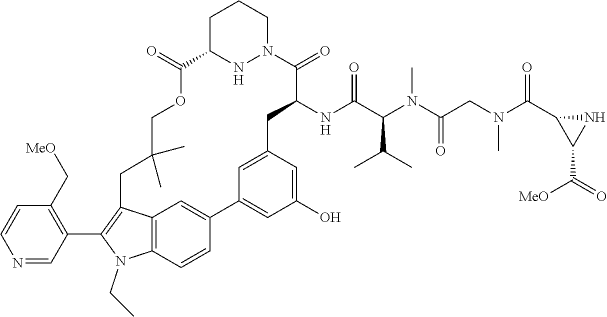 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C(=O)OC)C(=O)N1CCC[C@H](N1)C(=O)OCC(C)(C)C2] |
| C50 | 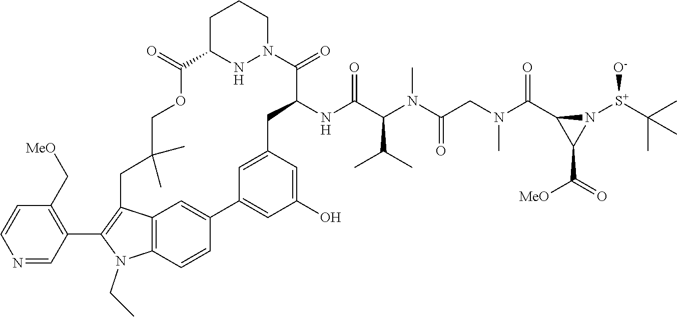 |
| C51 | 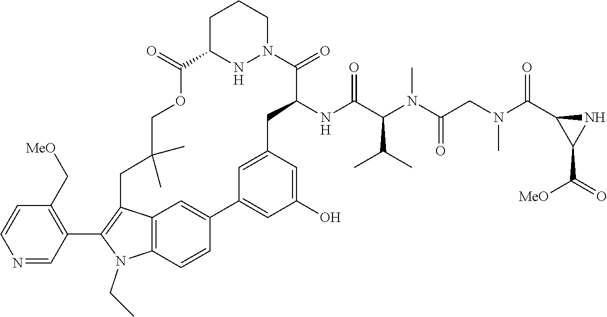 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C(=O)OC)C(=O)N1CCC[C@H](N1)C(=O)OCC(C)(C)C2] |
| C52 | 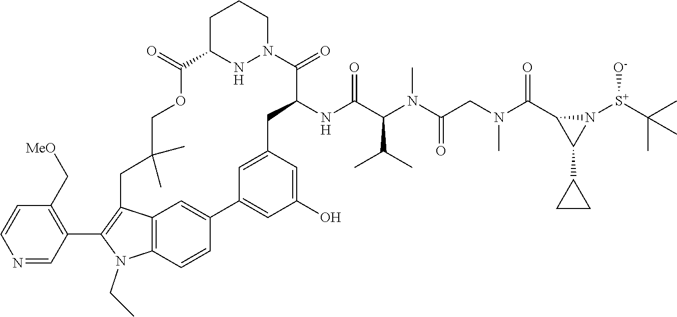 |
| C53 | 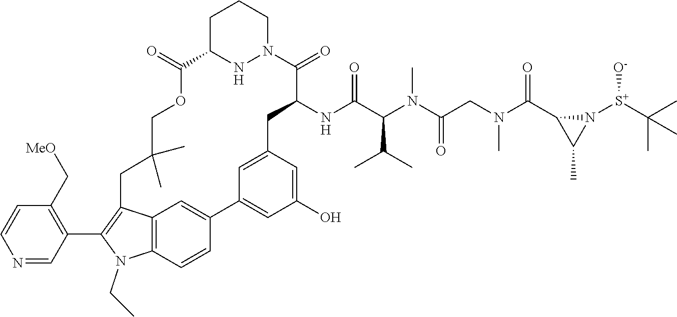 |
| C54 | 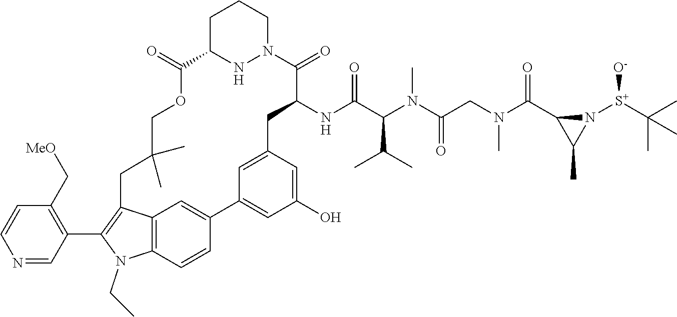 |
| C55 | 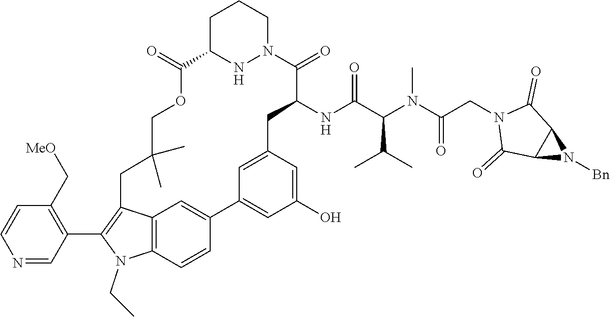 |
| C56* | 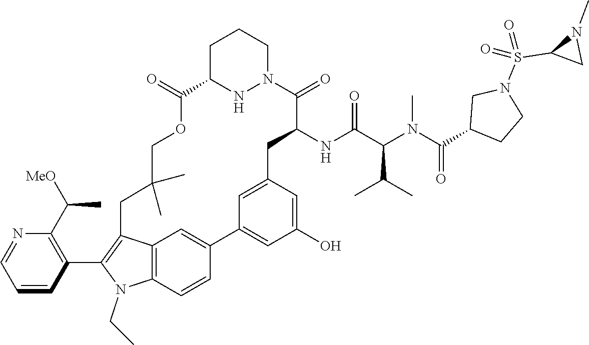 |
| C57* | 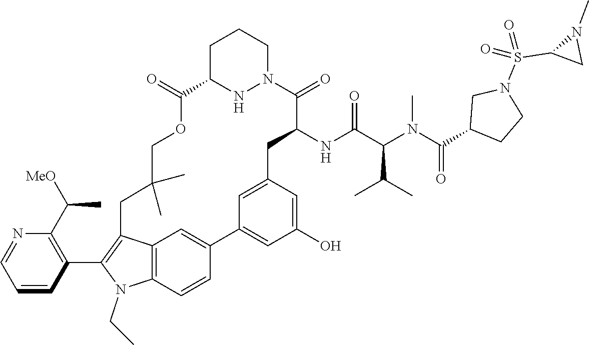 |
| C58 | 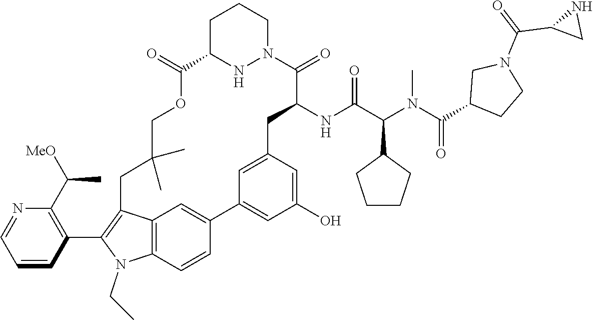 |
| C59 | 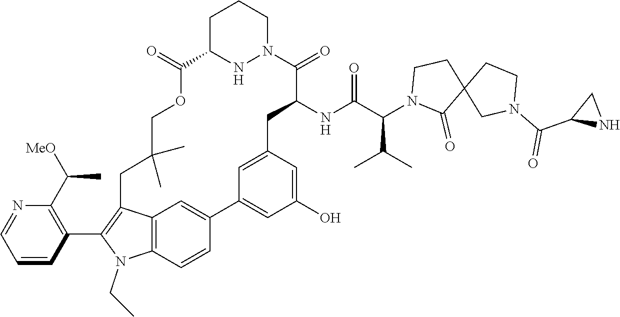 |
| C60 | 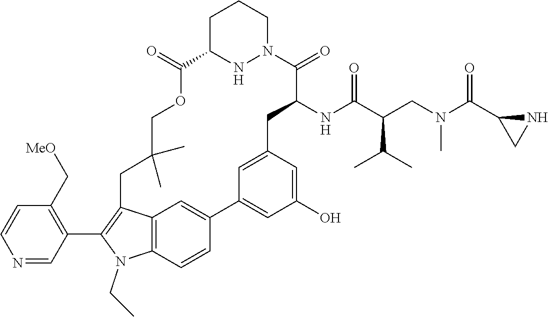 [SMILES: CCn1c(-c2cnccc2COC)c2c3cc(ccc31)-c1cc(O)cc(c1)C[C@H](NC(=O)[C@@H](CN(C)C(=O)[C@@H]1CN1)C(C)C)C(=O)N1CCC[C@H](N1)C(=O)OCC(C)(C)C2] |
| C61 A and B | 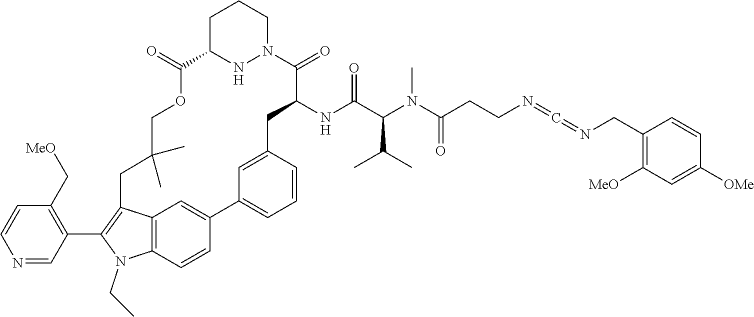 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccc(OC)cc1OC)C(=O)N1CCC[C@H](N1)C(=O)OCC(C)(C)C2] |
| C62A and B | 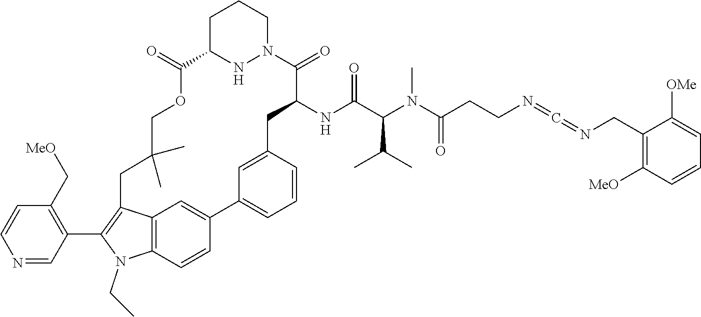 [SMILES: CCn1c(-c2cnccc2COC)c2c3cc(ccc31)-c1cccc(c1)C[C@H](NC(=O)[C@H](C(C)C)N(C)C(=O)CCN=C=NCc1c(OC)cccc1OC)C(=O)N1CCC[C@H](N1)C(=O)OCC(C)(C)C2] |
| C63 | 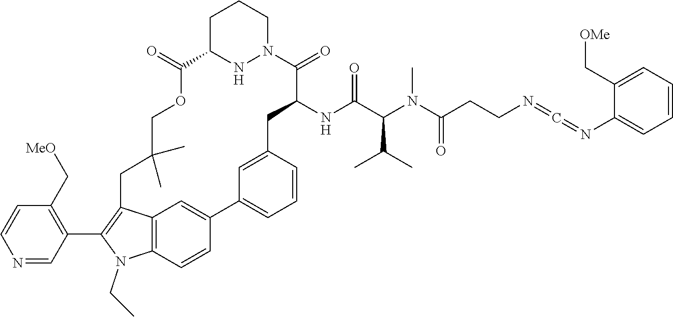 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1COC)C(=O)N1CCC[C@H](N1)C(=O)OCC(C)(C)C2] |
| C64 A and B | 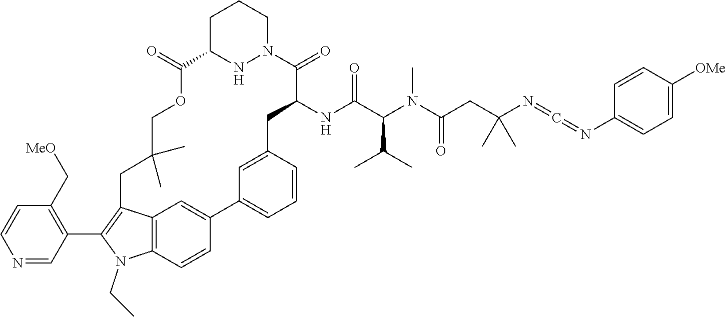 [SMILES: CCn1c(-c2cnccc2COC)c2c3cc(ccc31)-c1cccc(c1)C[C@H](NC(=O)[C@H](C(C)C)N(C)C(=O)CC(C)(C)N=C=Nc1ccc(OC)cc1)C(=O)N1CCC[C@H](N1)C(=O)OCC(C)(C)C2] |
| C65 A and B | 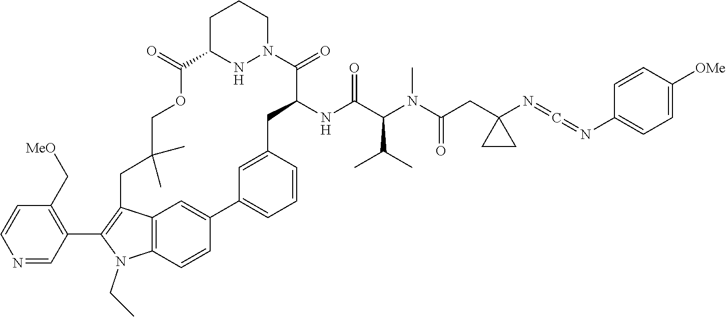 [SMILES: CCn1c(-c2cnccc2COC)c2c3cc(ccc31)-c1cccc(c1)C[C@H](NC(=O)[C@H](C(C)C)N(C)C(=O)CC1(N=C=Nc3ccc(OC)cc3)CC1)C(=O)N1CCC[C@H](N1)C(=O)OCC(C)(C)C2] |
| C66 | 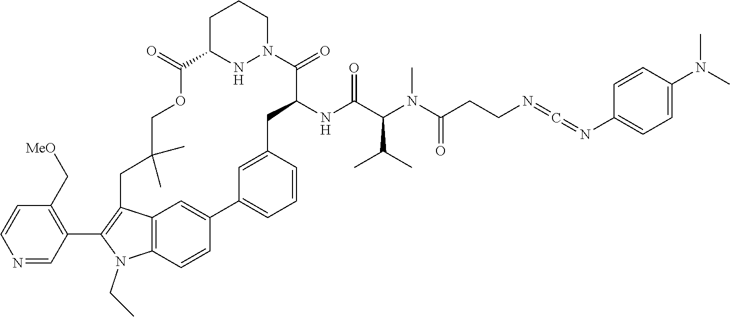 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(N(C)C)cc1)C(=O)N1CCC[C@H](N1)C(=O)OCC(C)(C)C2] |
| C67 | 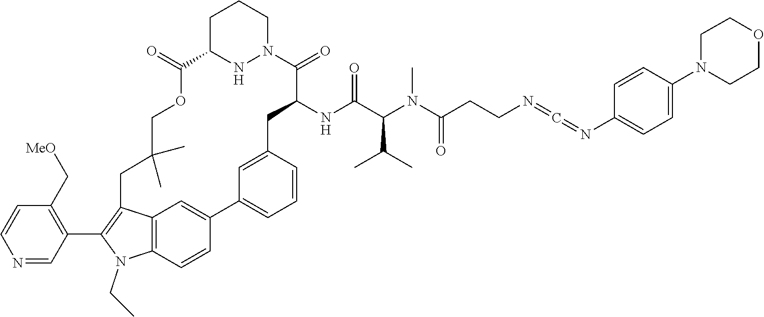 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(N3CCOCC3)cc1)C(=O)N1CCC[C@H](N1)C(=O)OCC(C)(C)C2] |
| C68 A and B | 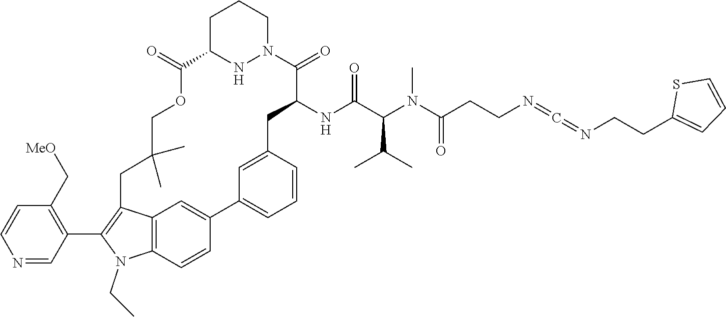 [SMILES: CCn1c(-c2cnccc2COC)c2c3cc(ccc31)-c1cccc(c1)C[C@H](NC(=O)[C@H](C(C)C)N(C)C(=O)CCN=C=NCCc1cccs1)C(=O)N1CCC[C@H](N1)C(=O)OCC(C)(C)C2] |
| C69 A and B | 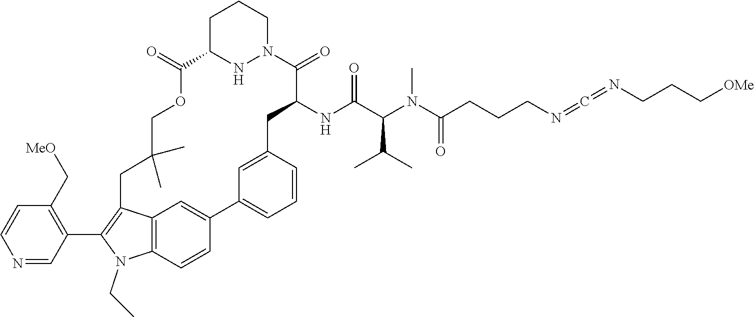 [SMILES: CCn1c(-c2cnccc2COC)c2c3cc(ccc31)-c1cccc(c1)C[C@H](NC(=O)[C@H](C(C)C)N(C)C(=O)CCCN=C=NCCCOC)C(=O)N1CCC[C@H](N1)C(=O)OCC(C)(C)C2] |
| C70 A and B | 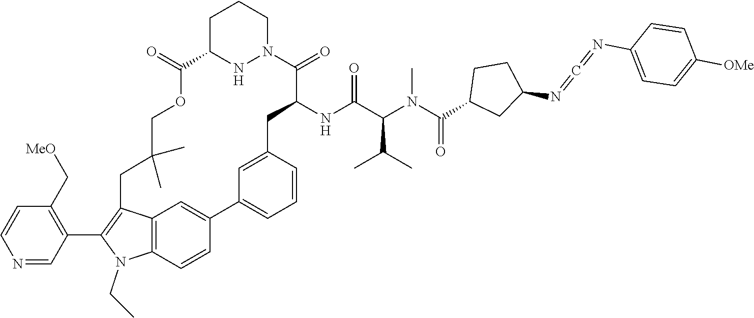 [SMILES: CCn1c(-c2cnccc2COC)c2c3cc(ccc31)-c1cccc(c1)C[C@H](NC(=O)[C@H](C(C)C)N(C)C(=O)[C@@H]1CC[C@@H](N=C=Nc3ccc(OC)cc3)C1)C(=O)N1CCC[C@H](N1)C(=O)OCC(C)(C)C2] |
| C71 A and B | 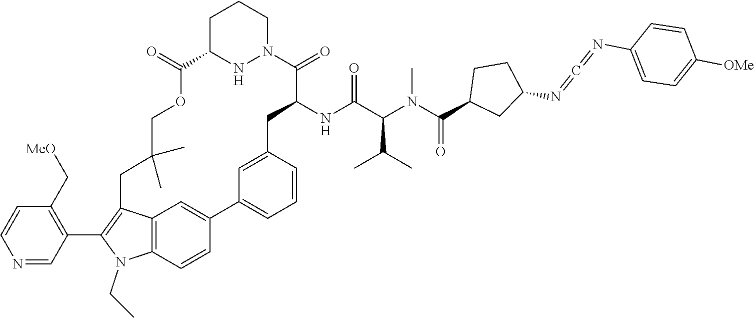 [SMILES: CCn1c(-c2cnccc2COC)c2c3cc(ccc31)-c1cccc(c1)C[C@H](NC(=O)[C@H](C(C)C)N(C)C(=O)[C@H]1CC[C@H](N=C=Nc3ccc(OC)cc3)C1)C(=O)N1CCC[C@H](N1)C(=O)OCC(C)(C)C2] |
| C72 A and B | 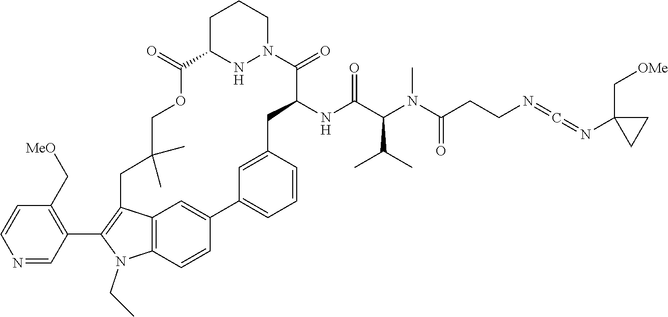 [SMILES: CCn1c(-c2cnccc2COC)c2c3cc(ccc31)-c1cccc(c1)C[C@H](NC(=O)[C@H](C(C)C)N(C)C(=O)CCN=C=NC1(COC)CC1)C(=O)N1CCC[C@H](N1)C(=O)OCC(C)(C)C2] |
| C73 | 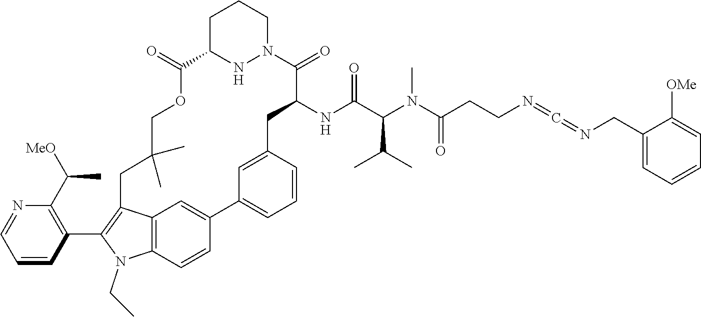 |
| C74 | 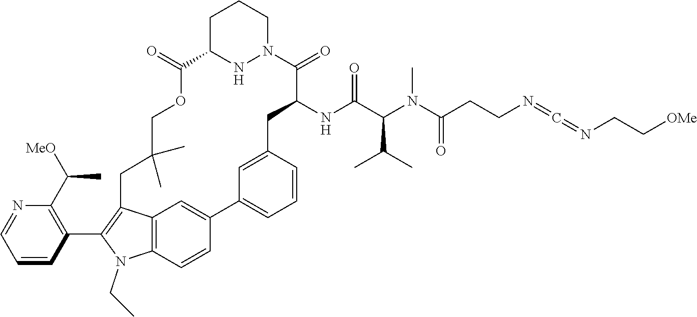 |
| C75 | 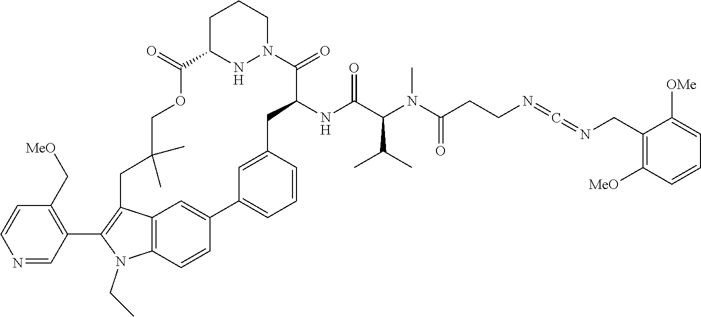 [SMILES: CCn1c(-c2cnccc2COC)c2c3cc(ccc31)-c1cccc(c1)C[C@H](NC(=O)[C@H](C(C)C)N(C)C(=O)CCN=C=NCc1c(OC)cccc1OC)C(=O)N1CCC[C@H](N1)C(=O)OCC(C)(C)C2] |
| C76 | 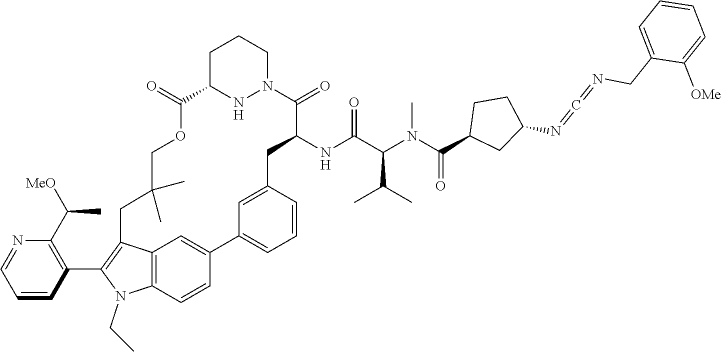 |
| C77 | 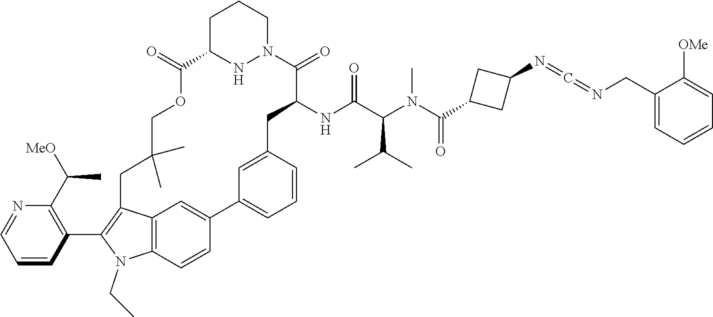 |
| C78 | 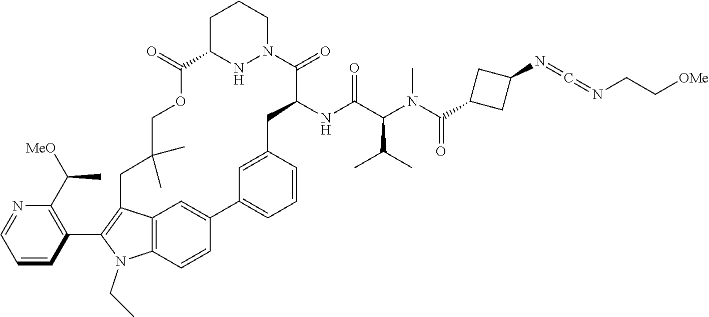 |
| C79 A and B | 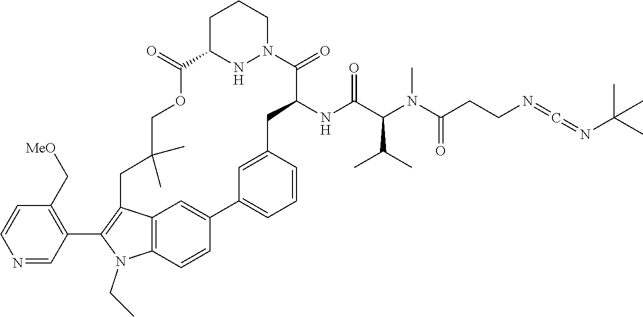 [SMILES: CCn1c(-c2cnccc2COC)c2c3cc(ccc31)-c1cccc(c1)C[C@H](NC(=O)[C@H](C(C)C)N(C)C(=O)CCN=C=NC(C)(C)C)C(=O)N1CCC[C@H](N1)C(=O)OCC(C)(C)C2] |
| C80 | 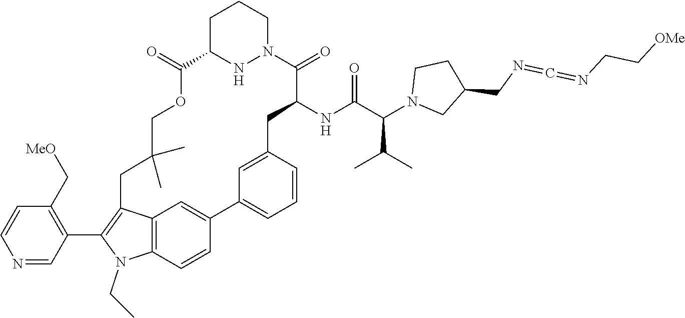 [SMILES: CCn1c(-c2cnccc2COC)c2c3cc(ccc31)-c1cccc(c1)C[C@H](NC(=O)[C@H](C(C)C)N1CC[C@@H](CN=C=NCCOC)C1)C(=O)N1CCC[C@H](N1)C(=O)OCC(C)(C)C2] |
| C81 | 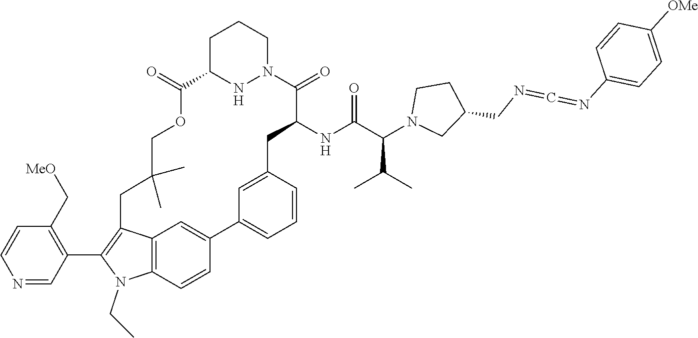 [SMILES: CCn1c(-c2cnccc2COC)c2c3cc(ccc31)-c1cccc(c1)C[C@H](NC(=O)[C@H](C(C)C)N1CC[C@H](CN=C=Nc3ccc(OC)cc3)C1)C(=O)N1CCC[C@H](N1)C(=O)OCC(C)(C)C2] |
| C82 A and B | 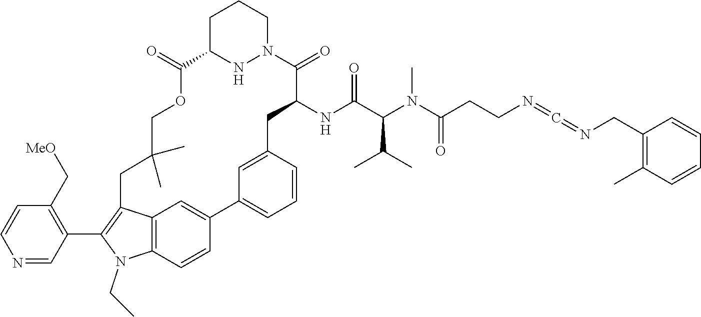 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccccc1C)C(=O)N1CCC[C@H](N1)C(=O)OCC(C)(C)C2] |
| C83 | 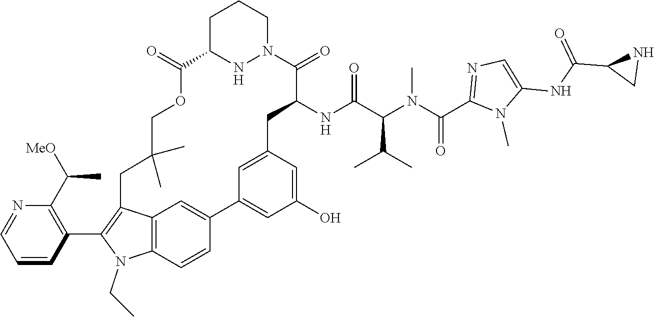 |
| C84 | 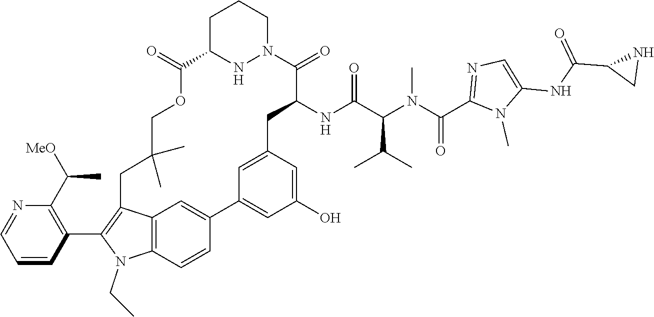 |
| C85 | 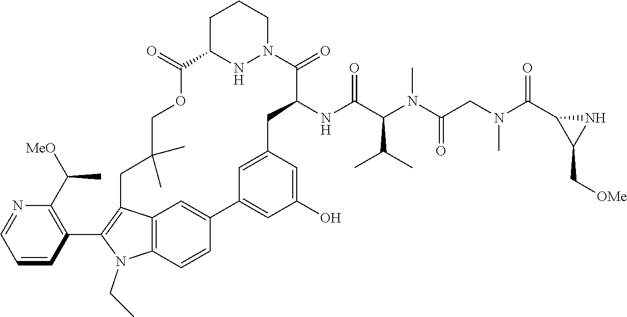 |
| C86 | 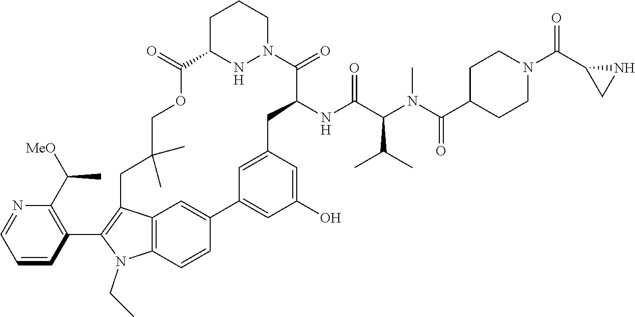 |
| C87 | 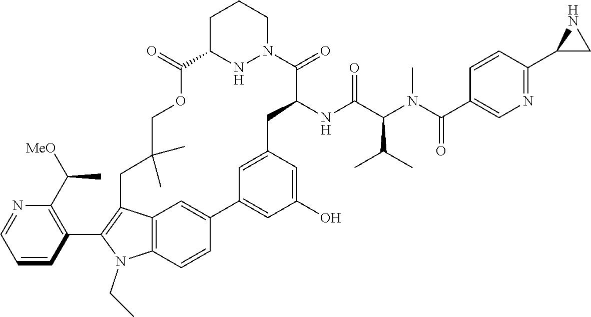 |
| C88 | 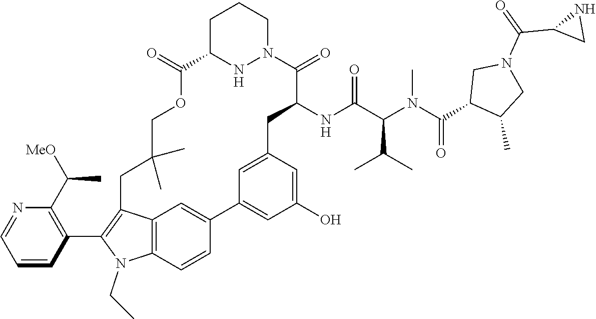 |
| C89 | 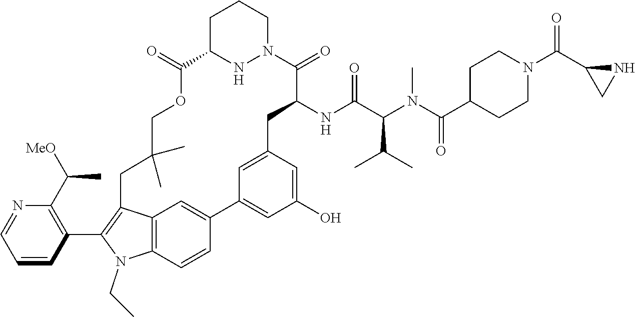 |
| C90 | 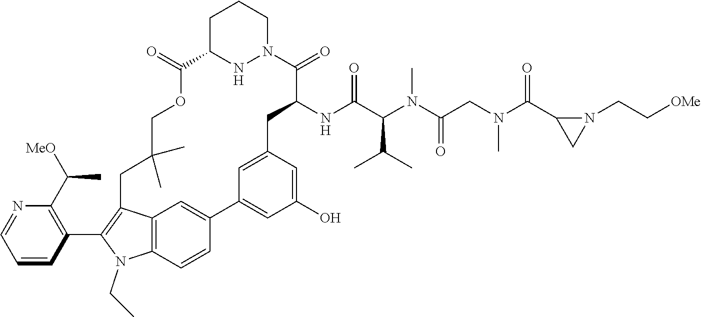 |
| C91* | 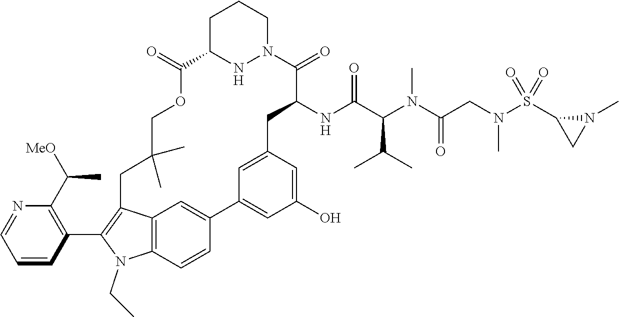 |
| C92* | 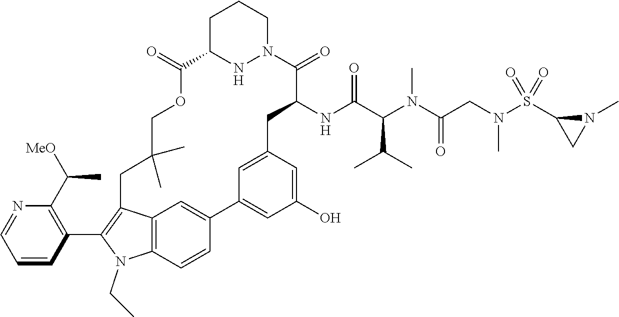 |
| C93 | 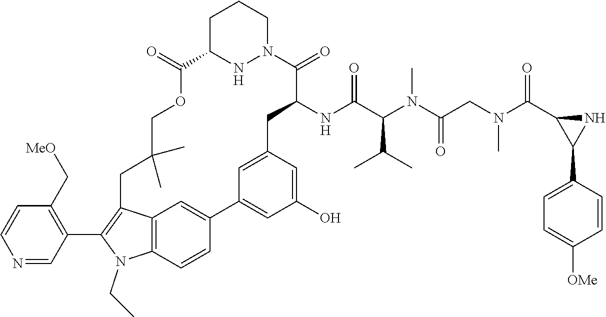 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1c1ccc(OC)cc1)C(=O)N1CCC[C@H](N1)C(=O)OCC(C)(C)C2] |
| C94 | 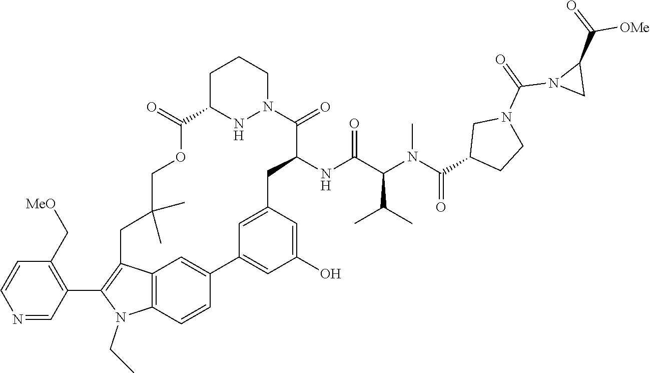 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@@H]3C(=O)OC)C1)C(=O)N1CCC[C@H](N1)C(=O)OCC(C)(C)C2] |
| C95 | 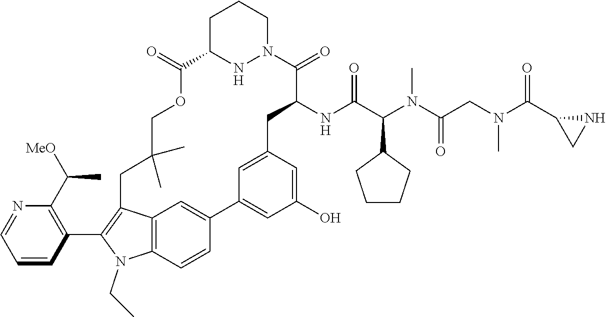 |
| C96 | 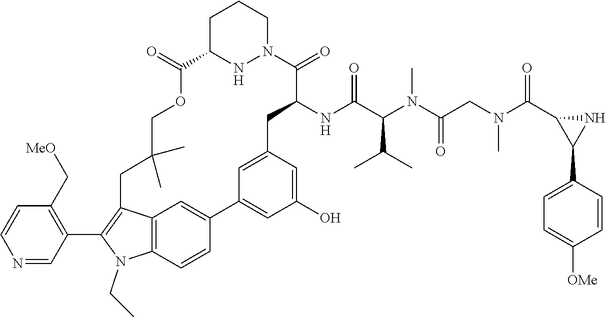 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1c1ccc(OC)cc1)C(=O)N1CCC[C@H](N1)C(=O)OCC(C)(C)C2] |
| C97 | 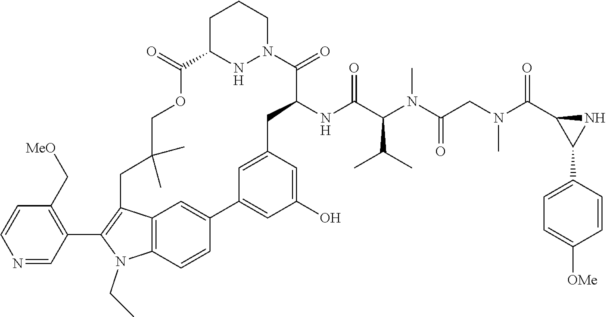 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1c1ccc(OC)cc1)C(=O)N1CCC[C@H](N1)C(=O)OCC(C)(C)C2] |
| C98 | 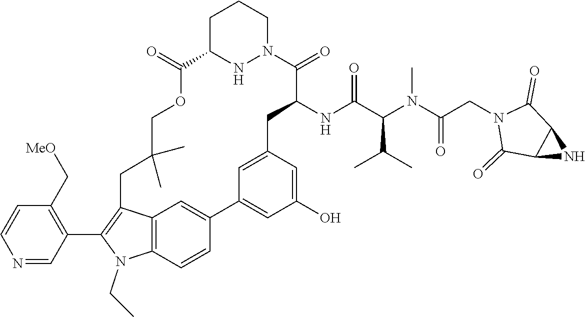 [SMILES: CCn1c(-c2cnccc2COC)c2c3cc(ccc31)-c1cc(O)cc(c1)C[C@H](NC(=O)[C@H](C(C)C)N(C)C(=O)CN1C(=O)[C@H]3N[C@H]3C1=O)C(=O)N1CCC[C@H](N1)C(=O)OCC(C)(C)C2] |
| C99 | 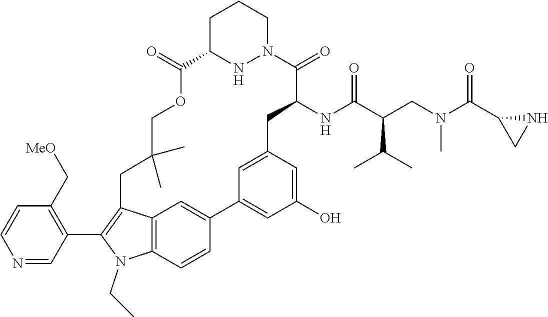 [SMILES: CCn1c(-c2cnccc2COC)c2c3cc(ccc31)-c1cc(O)cc(c1)C[C@H](NC(=O)[C@@H](CN(C)C(=O)[C@H]1CN1)C(C)C)C(=O)N1CCC[C@H](N1)C(=O)OCC(C)(C)C2] |
| C100 | 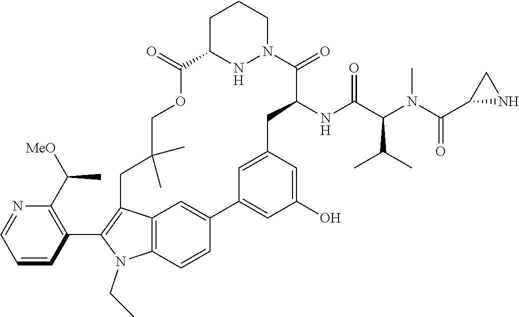 |
| C101 | 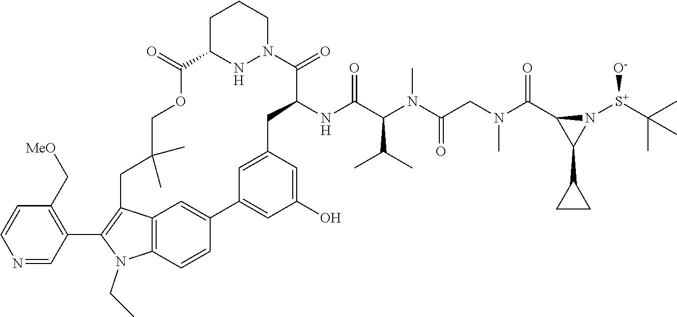 |
| C102 | 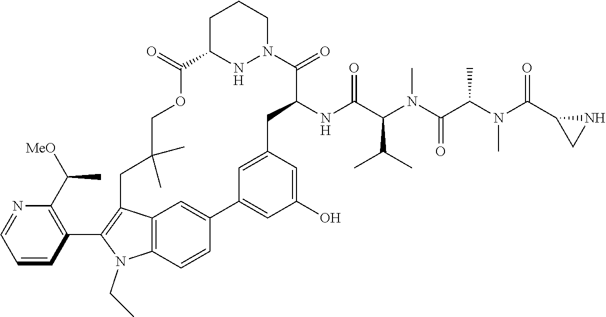 |
| C103 | 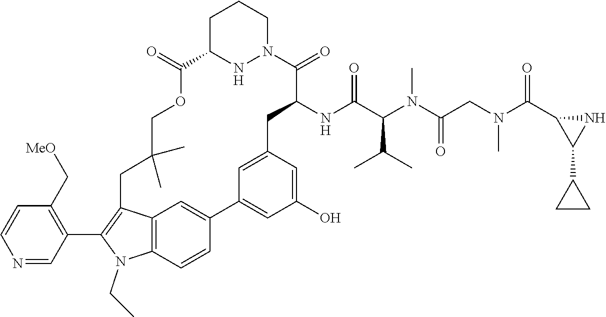 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C1CC1)C(=O)N1CCC[C@H](N1)C(=O)OCC(C)(C)C2] |
| C104 | 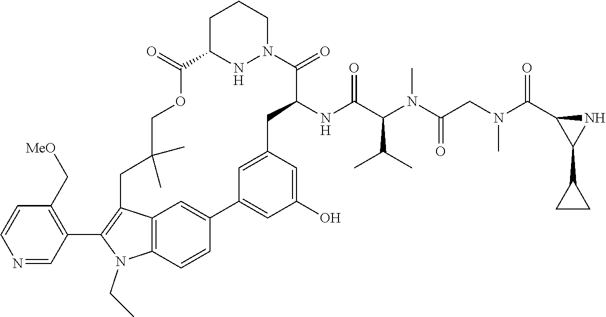 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C1CC1)C(=O)N1CCC[C@H](N1)C(=O)OCC(C)(C)C2] |
| C105 | 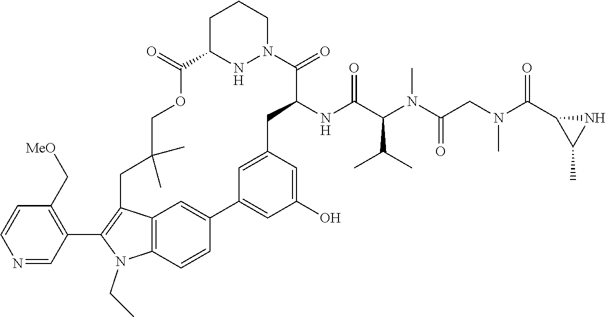 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C)C(=O)N1CCC[C@H](N1)C(=O)OCC(C)(C)C2] |
| C106 | 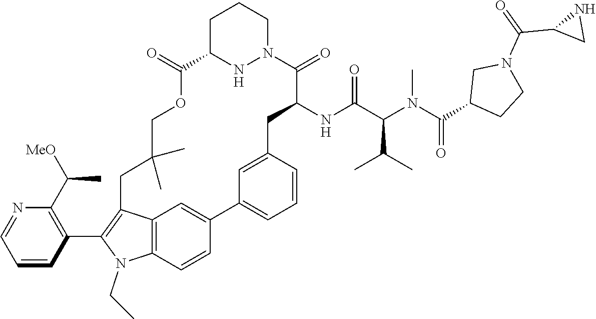 |
| C107 | 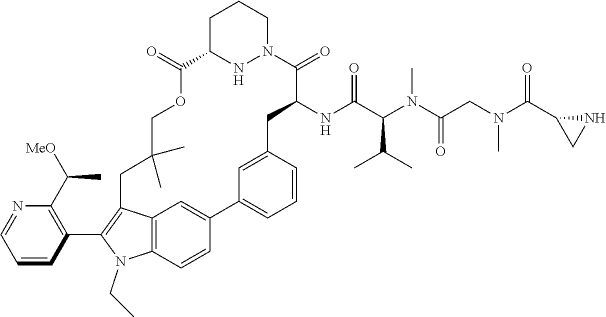 |
| C108* | 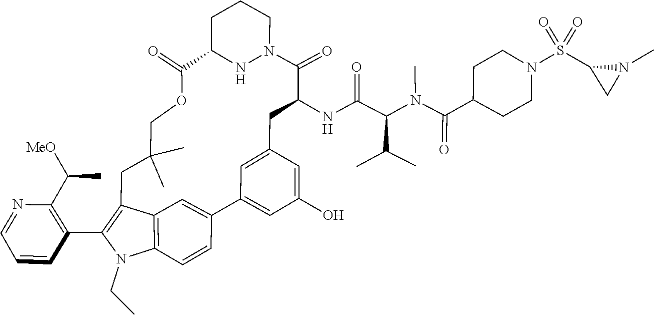 |
| C109 | 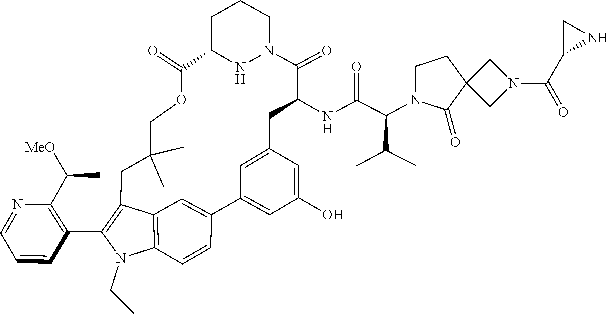 |
| C110 | 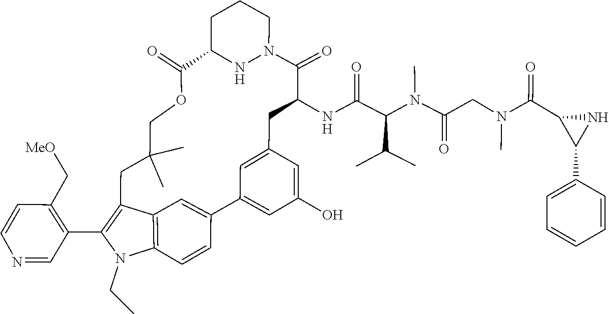 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccccc1)C(=O)N1CCC[C@H](N1)C(=O)OCC(C)(C)C2] |
| C111 | 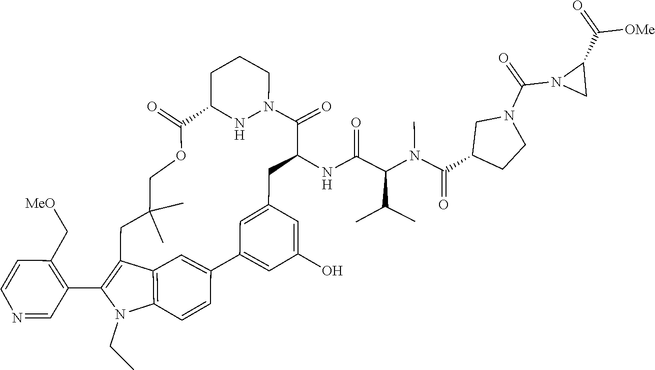 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@H]3C(=O)OC)C1)C(=O)N1CCC[C@H](N1)C(=O)OCC(C)(C)C2] |
| C112 | 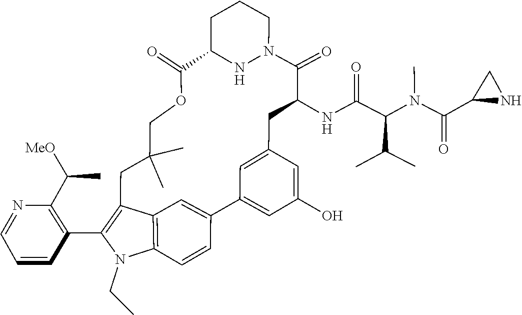 |
| C113 | 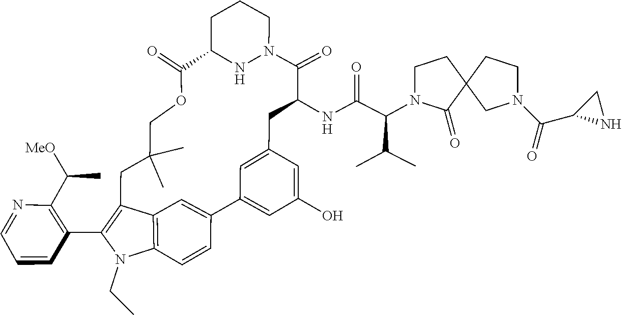 |
| C114 | 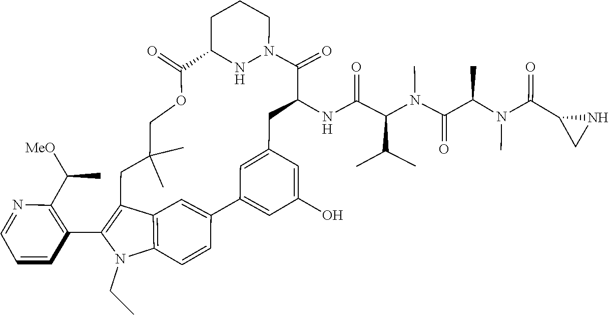 |
| C115 | 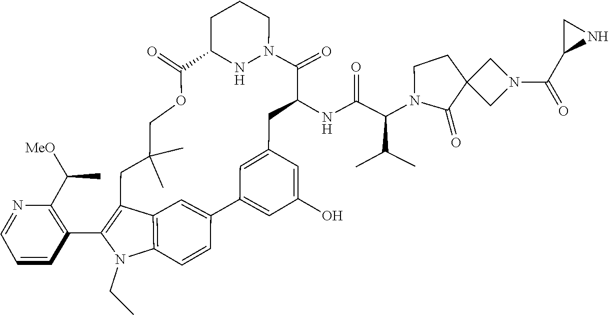 |
| C116 | 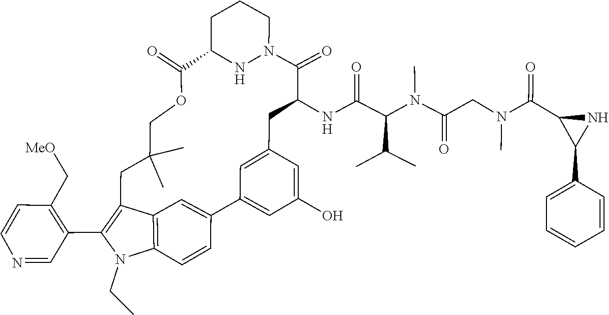 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1c1ccccc1)C(=O)N1CCC[C@H](N1)C(=O)OCC(C)(C)C2] |
| C117* | 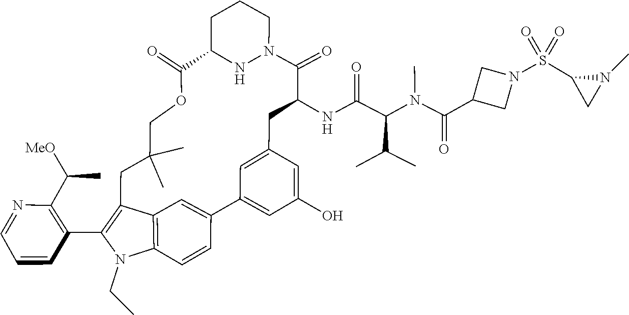 |
| C118* | 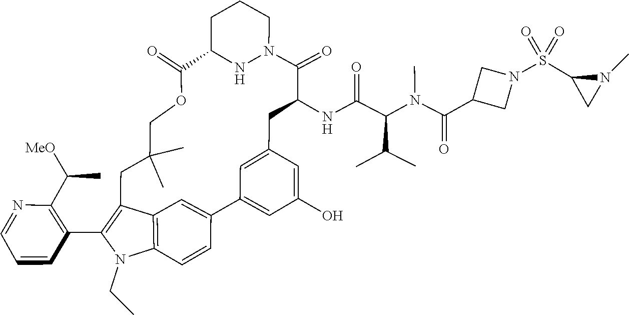 |
| C119* | 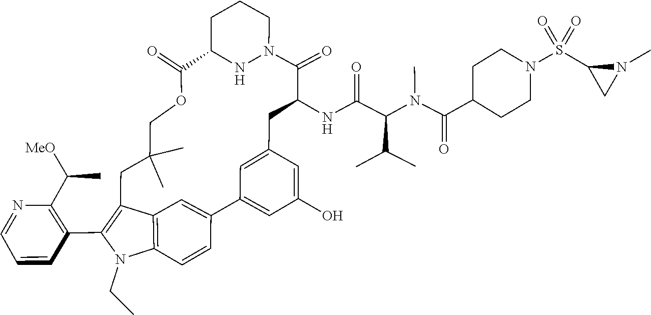 |
| C120 | 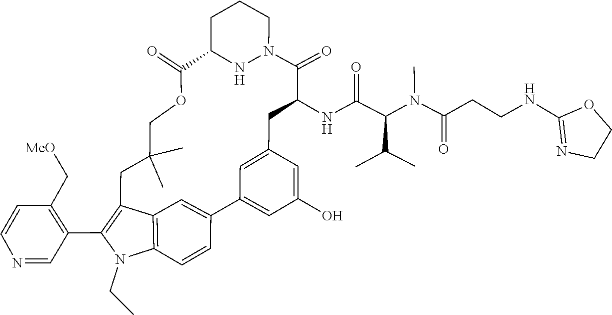 [SMILES: CCn1c(-c2cnccc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| C121 | 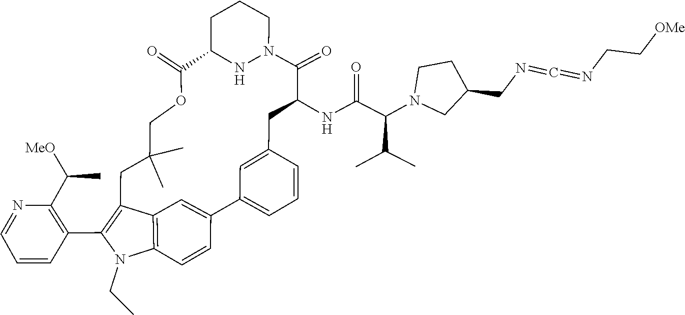 [SMILES: CCn1c(-c2cccnc2[C@H](C)OC)c2c3cc(ccc31)-c1cccc(c1)C[C@H](NC(=O)[C@H](C(C)C)N1CC[C@@H](CN=C=NCCOC)C1)C(=O)N1CCC[C@H](N1)C(=O)OCC(C)(C)C2] |
| C122 | 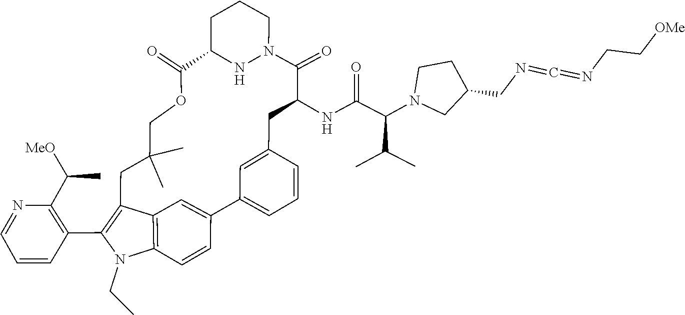 [SMILES: CCn1c(-c2cccnc2[C@H](C)OC)c2c3cc(ccc31)-c1cccc(c1)C[C@H](NC(=O)[C@H](C(C)C)N1CC[C@H](CN=C=NCCOC)C1)C(=O)N1CCC[C@H](N1)C(=O)OCC(C)(C)C2] |
| C123 | 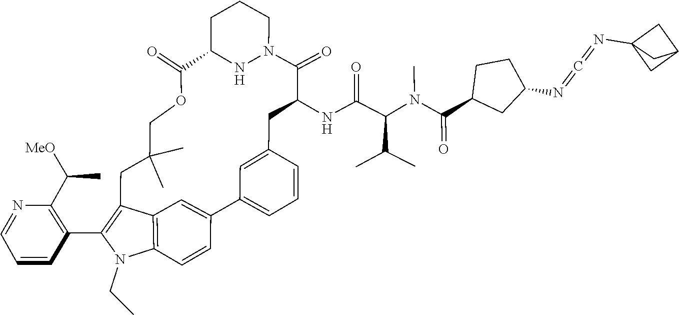 |
| C124 | 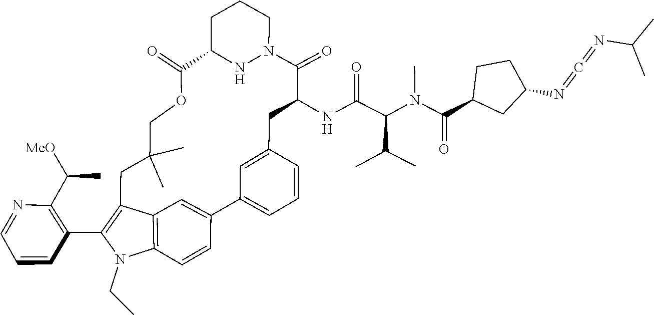 |
| C125 | 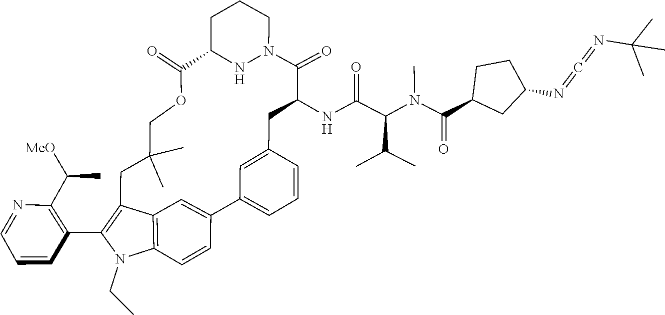 |
| C126 | 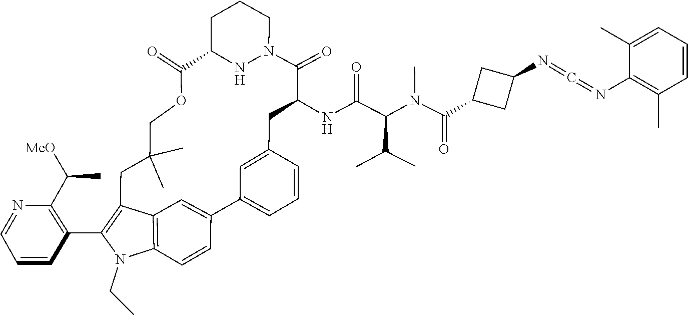 |
| C127 | 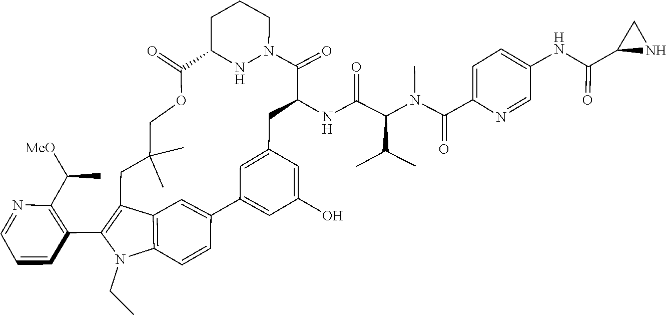 |
| C128 | 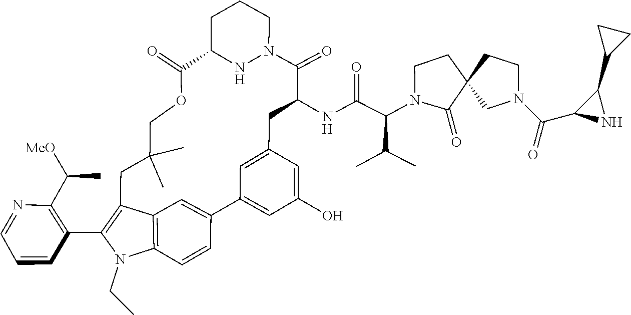 |
| C129 | 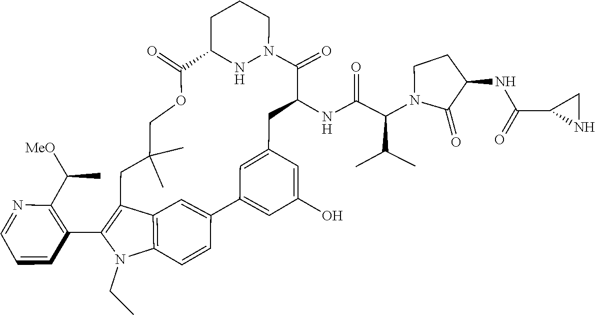 |
| C130 | 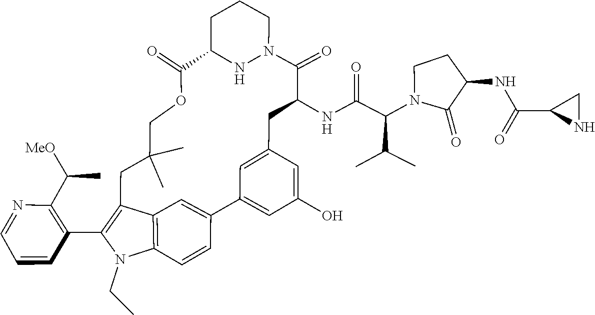 |
| C131 | 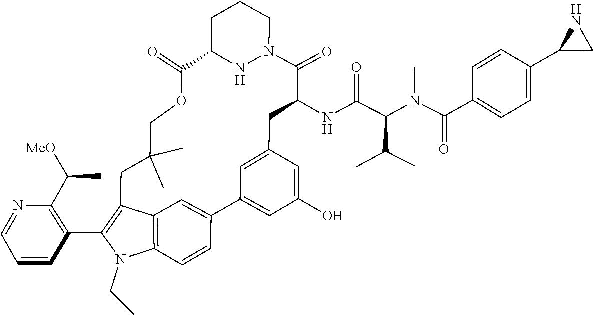 |
| C132 | 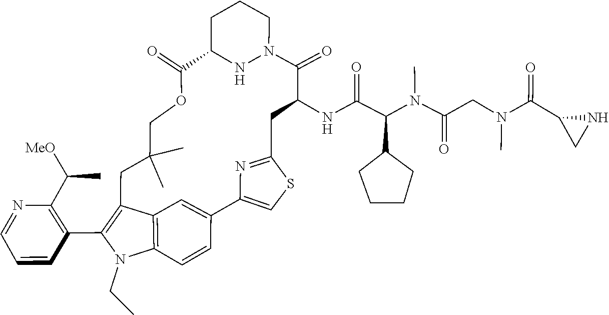 |
| C133 | 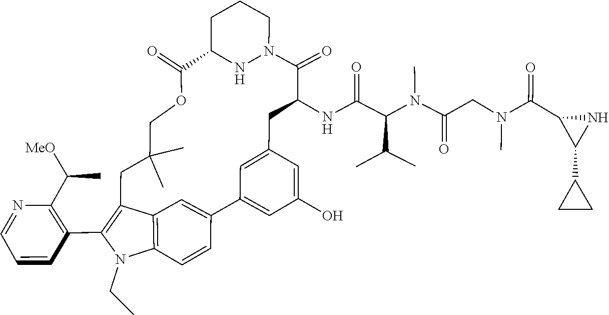 |
| C134 | 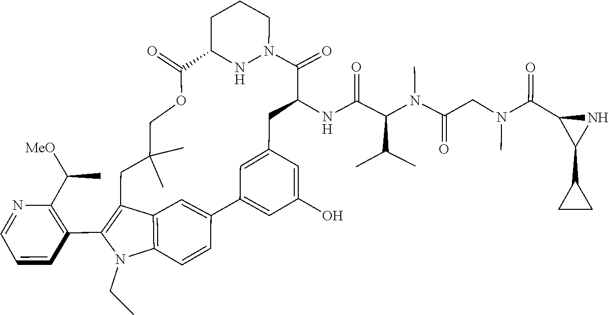 |
| C135 | 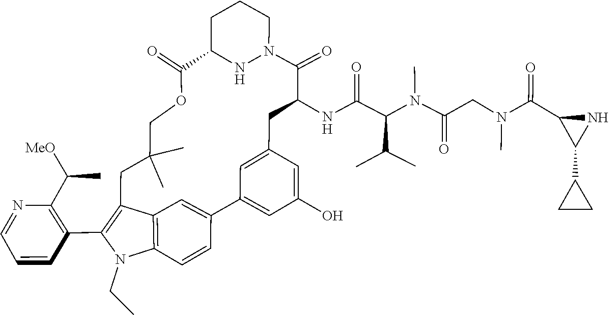 |
| C136 | 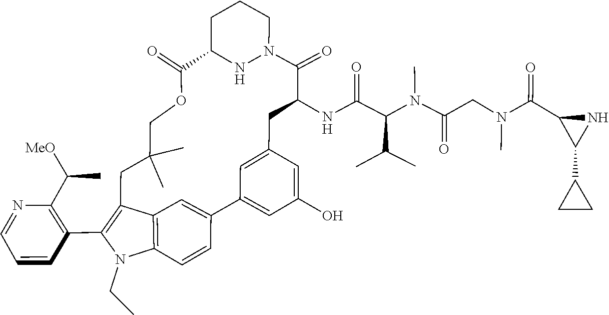 |
| C137 | 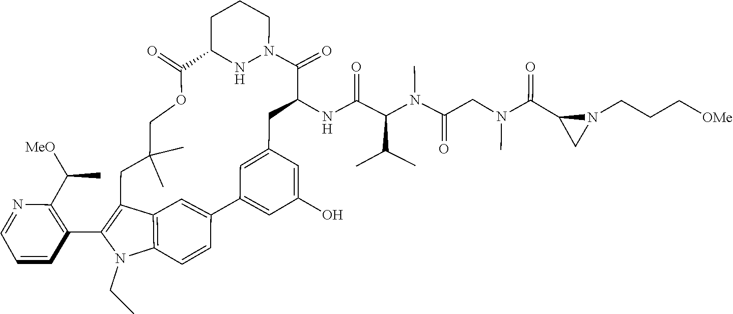 |
| C138 | 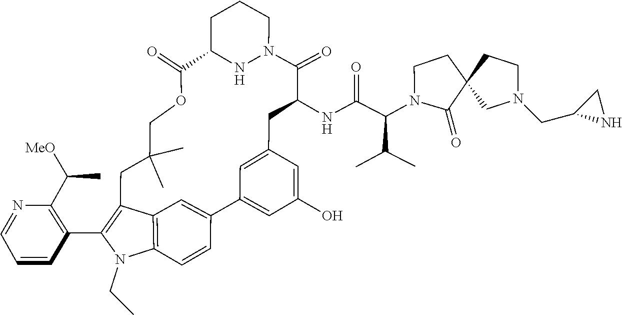 |
| C139 | 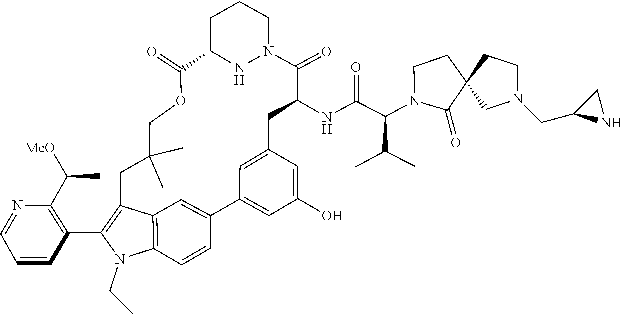 |
| C140 | 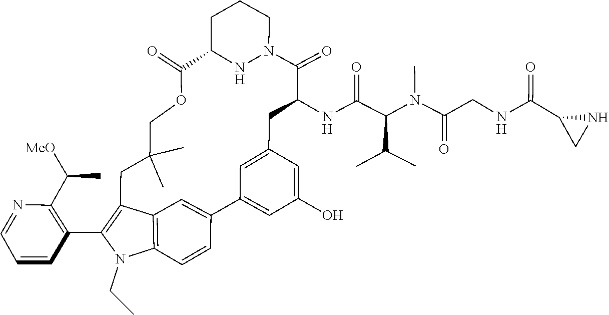 |
| C141 | 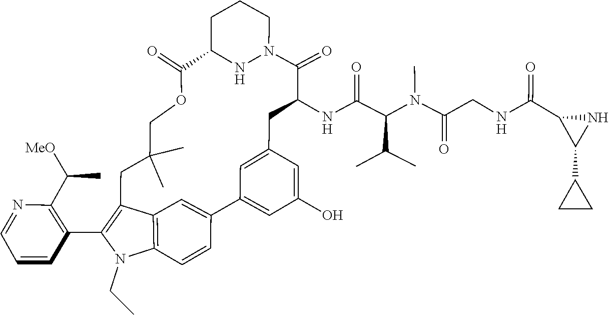 |
| C142 | 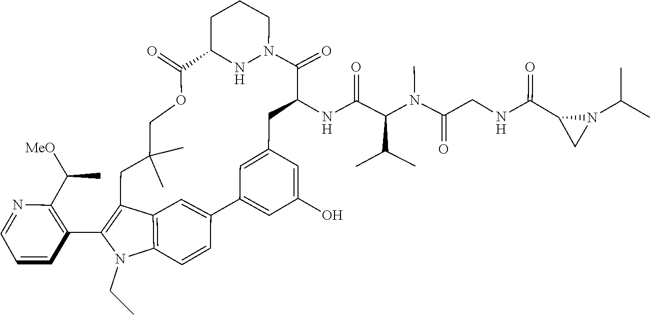 |
| C143 | 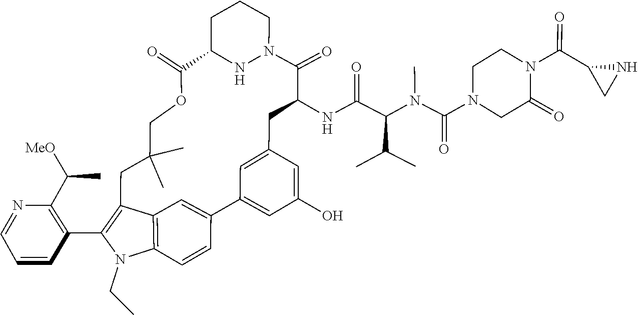 |
| C144 | 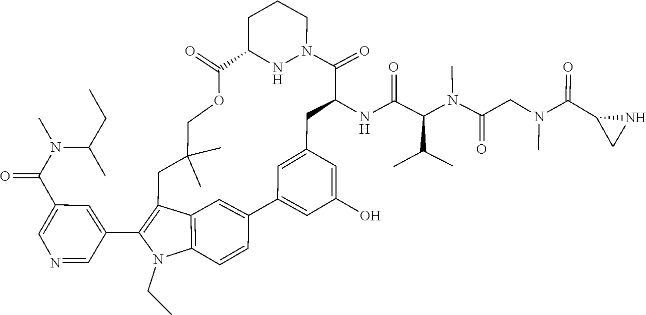 [SMILES: CCC(C)N(C)C(=O)c1cncc(-c2c3c4cc(ccc4n2CC)-c2cc(O)cc(c2)C[C@H](NC(=O)[C@H](C(C)C)N(C)C(=O)CN(C)C(=O)[C@H]2CN2)C(=O)N2CCC[C@H](N2)C(=O)OCC(C)(C)C3)c1] |
| C145 | 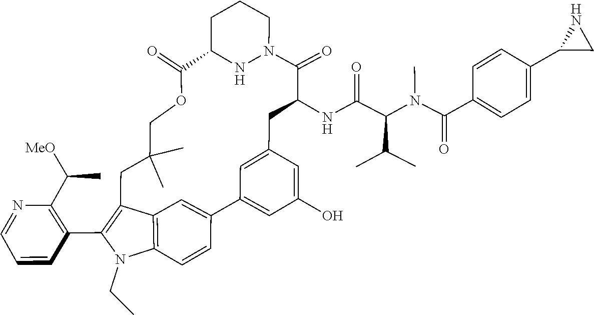 |
| C146 | 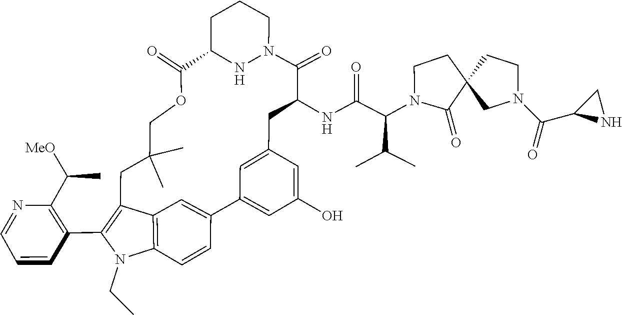 |
| C147 | 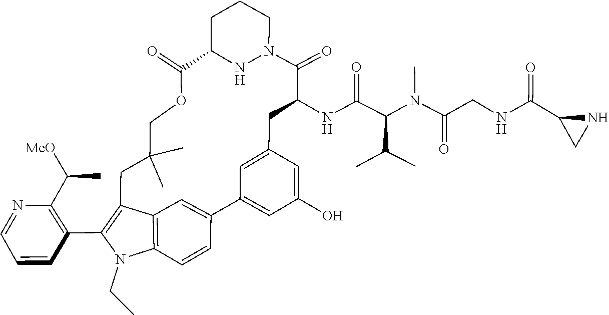 |
| C148 | 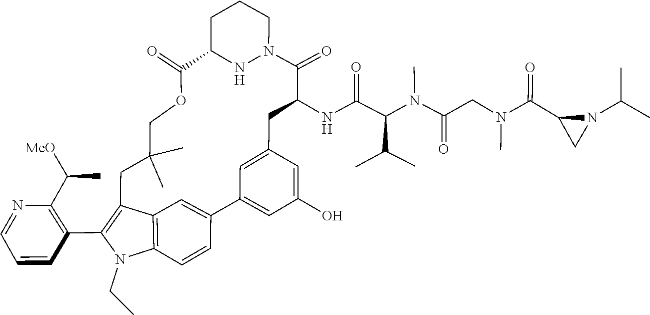 |
| C149 | 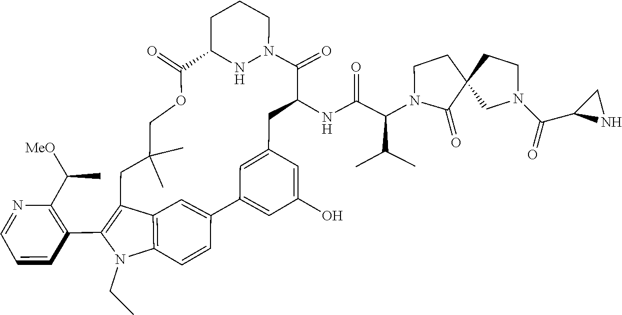 |
| C150 | 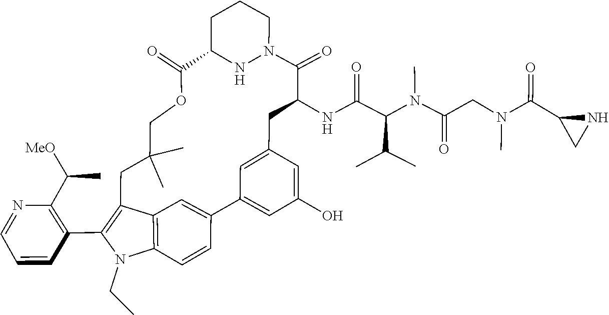 |
| C151 | 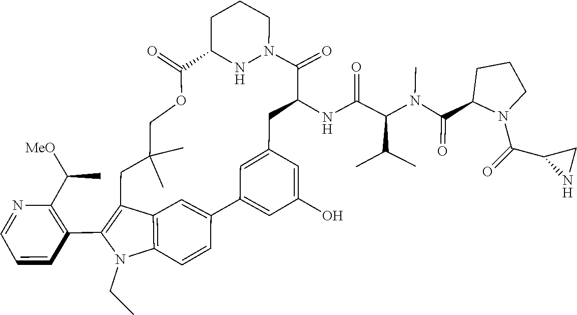 |
| C152 | 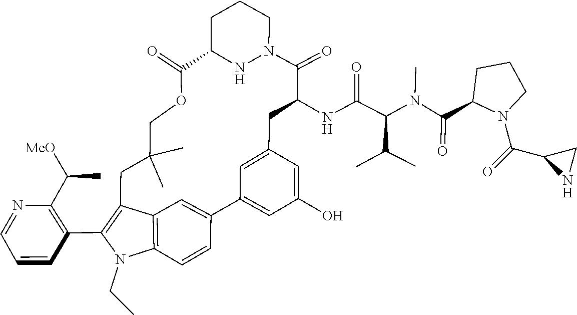 |
| C153 | 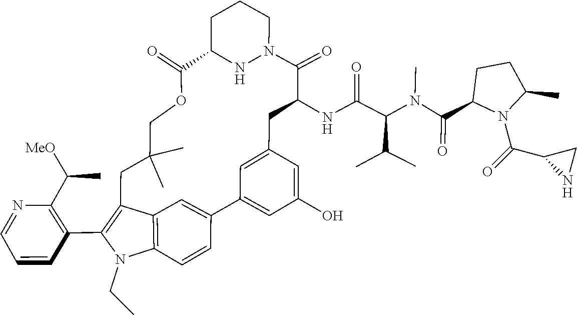 |
| C154 | 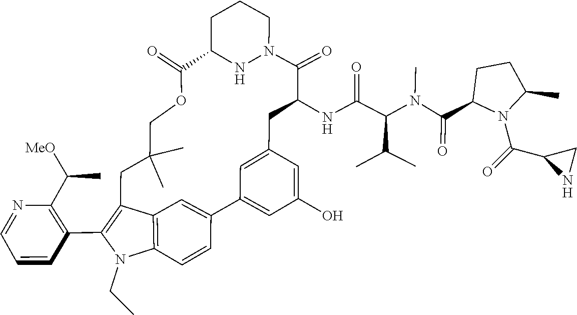 |
| C155 | 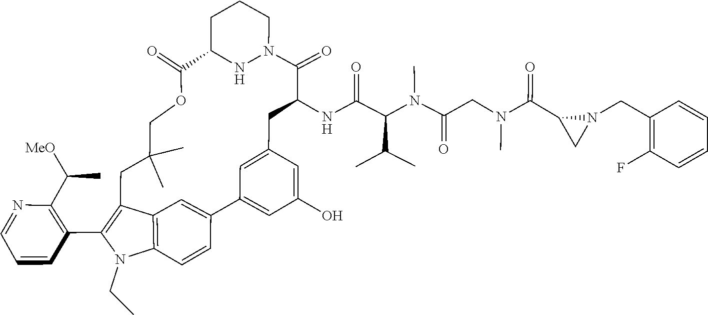 |
| C156 | 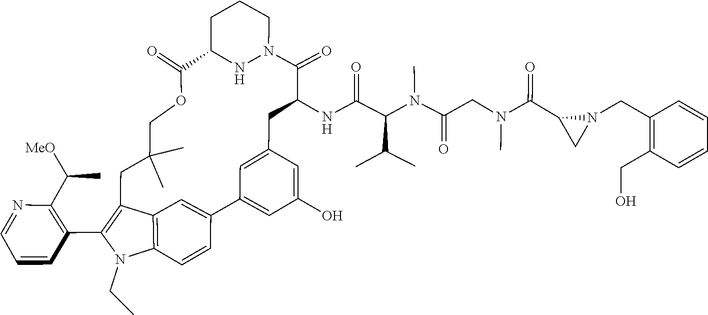 |
| C157 | 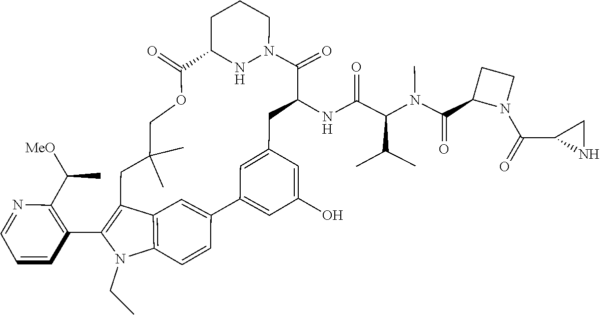 |
| C158 | 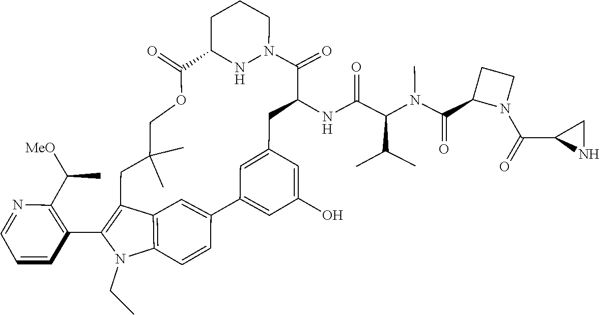 |
| C159 | 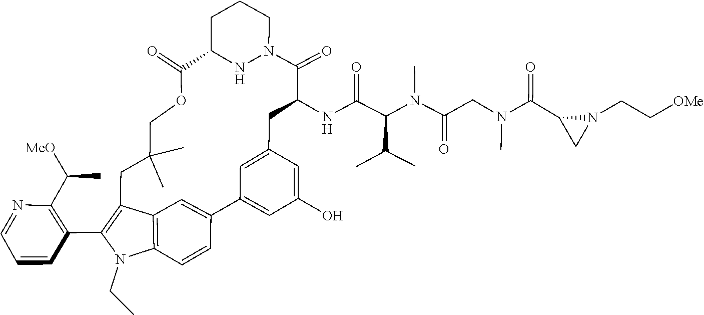 |
| C160 | 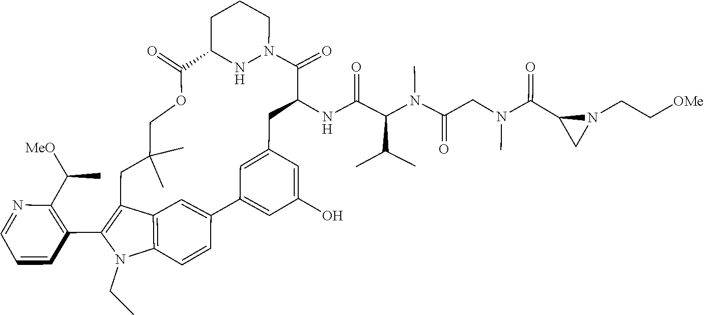 |
| C161 | 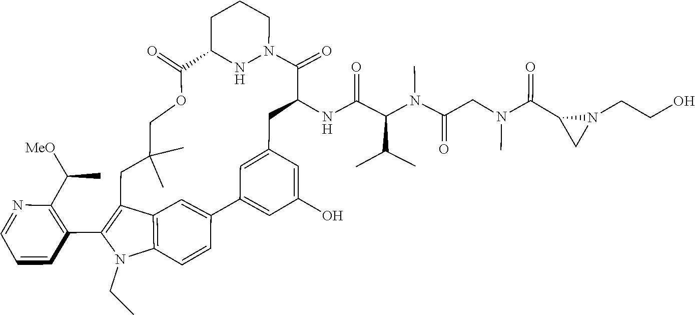 |
| C162 | 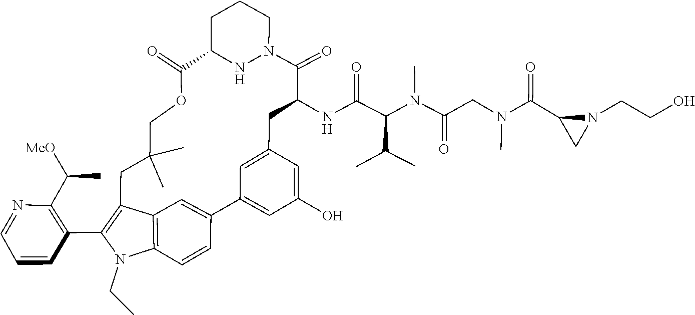 |
| C163 | 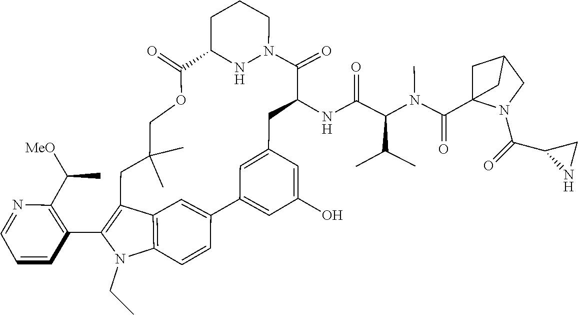 |
| C164 | 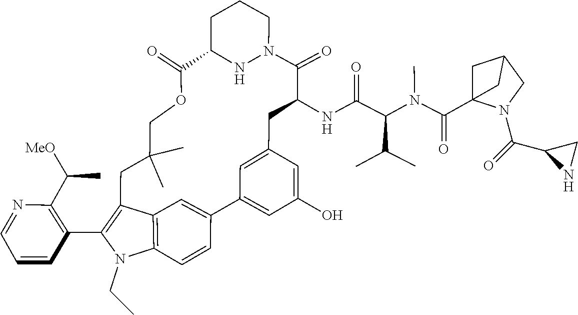 |
| C165 | 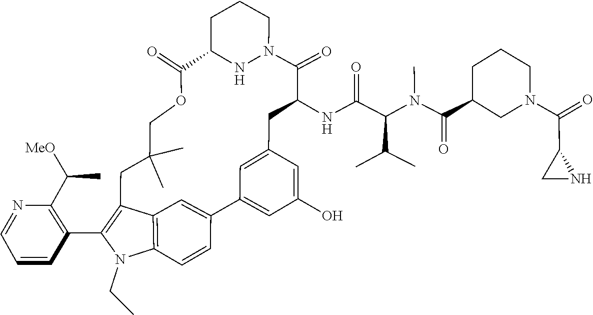 |
| C166 | 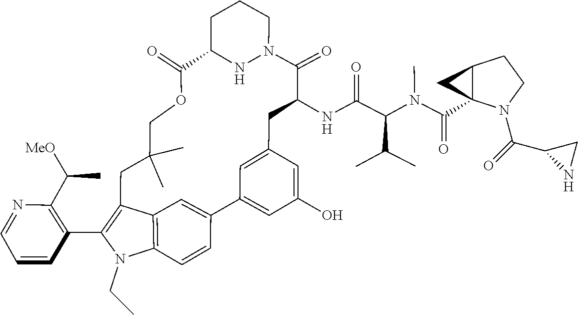 |
| C167 | 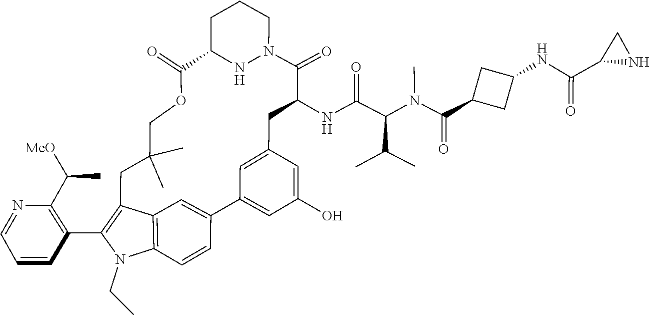 |
| C168 | 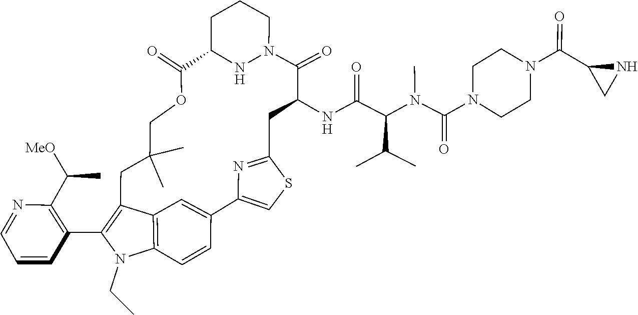 |
| C169 | 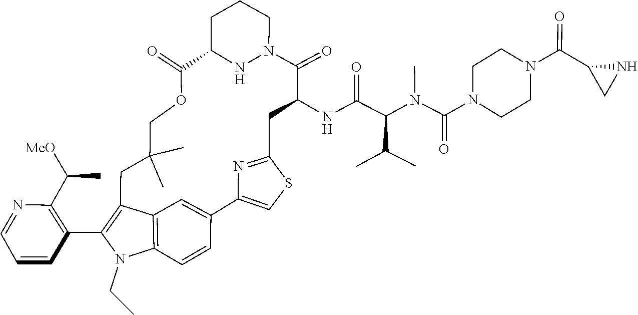 |
| C170 | 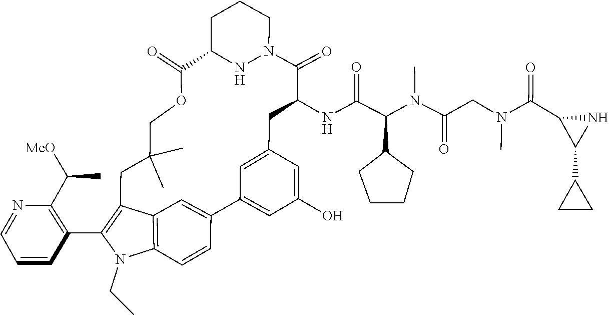 |
| C171 | 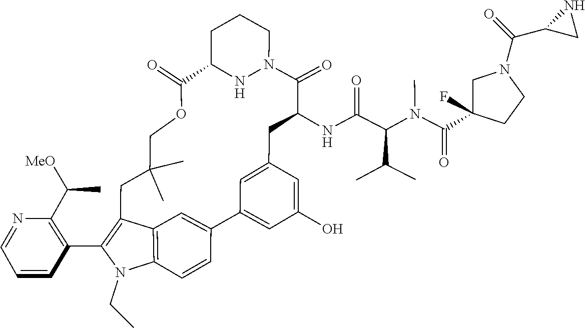 |
| C172 | 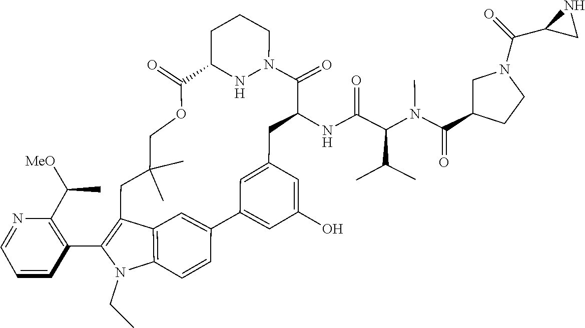 |
| C173 | 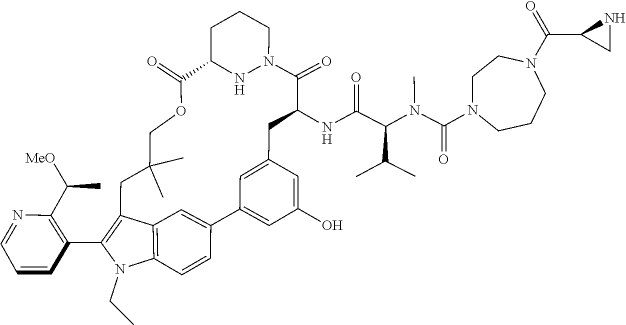 |
| C174 | 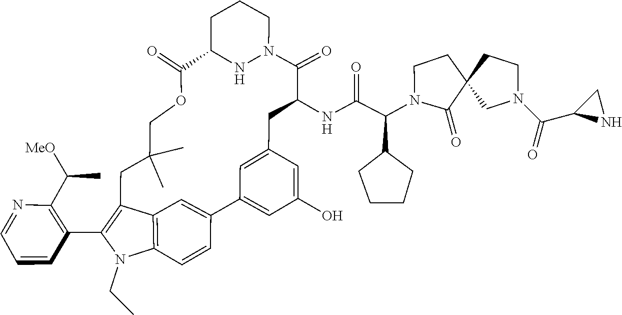 |
| C175 | 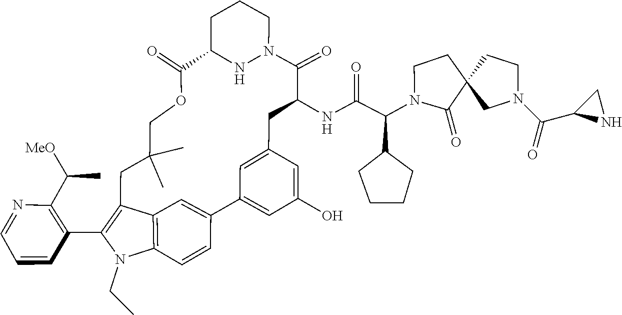 |
| C176 | 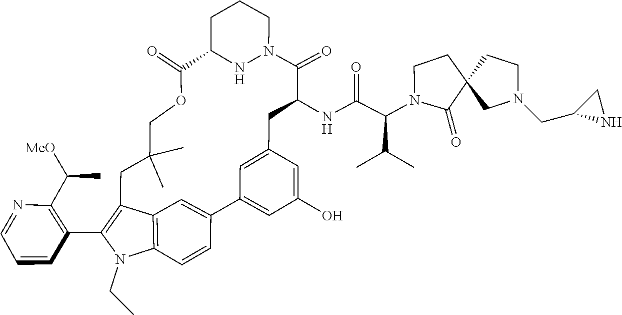 |
| C177 | 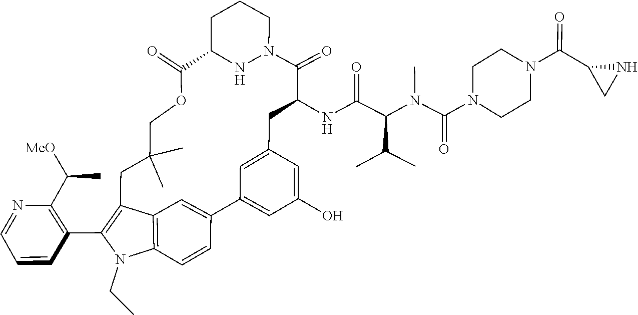 |
| C178 | 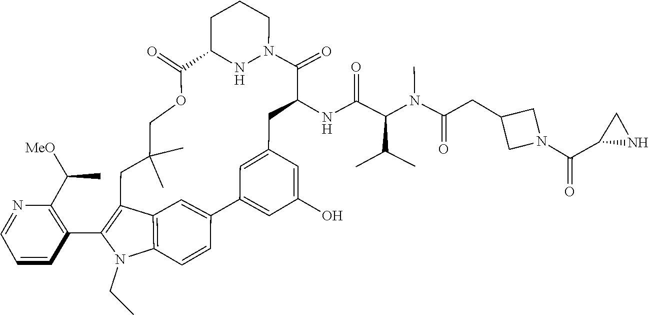 |
| C179 | 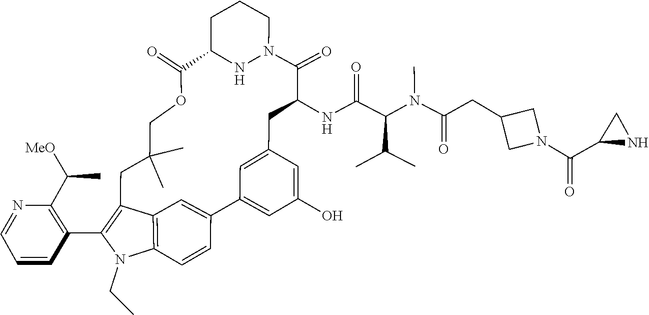 |
| C180 | 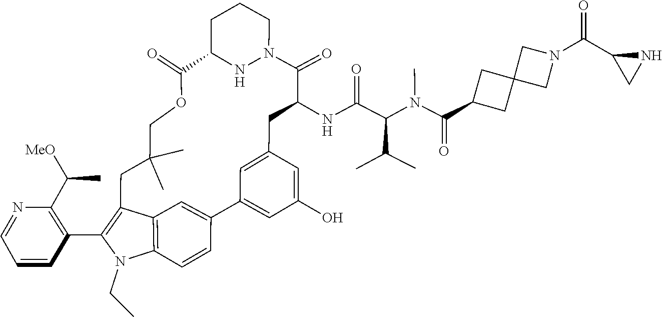 |
| C181 | 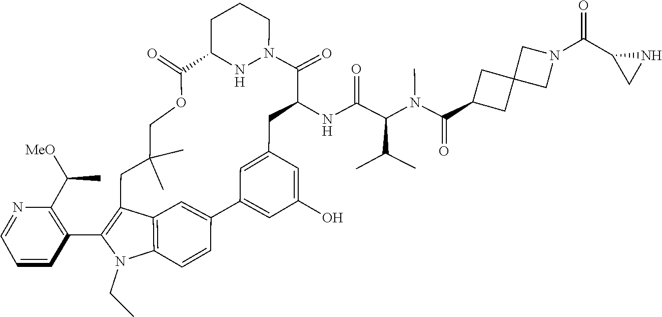 |
| C182 | 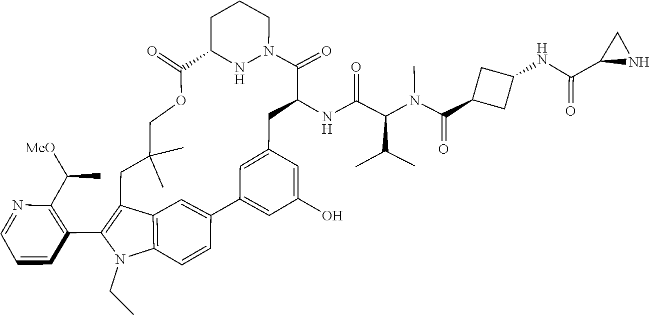 |
| C183 | 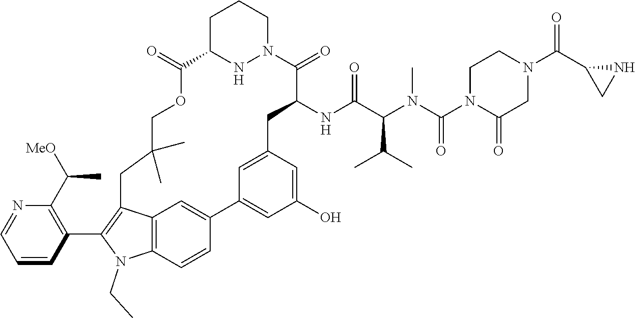 |
| C184 | 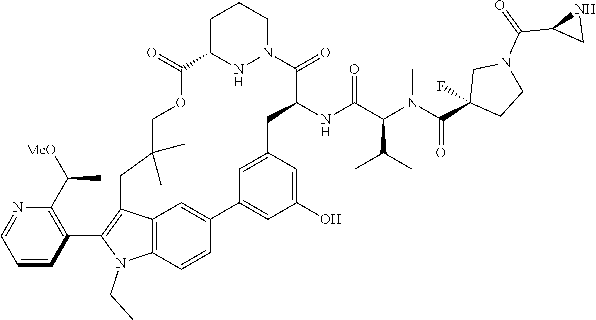 |
| C185 | 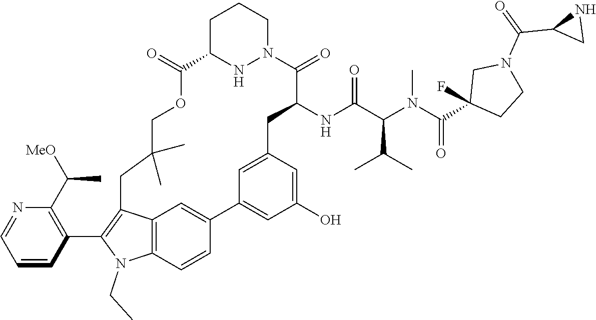 |
| C186 | 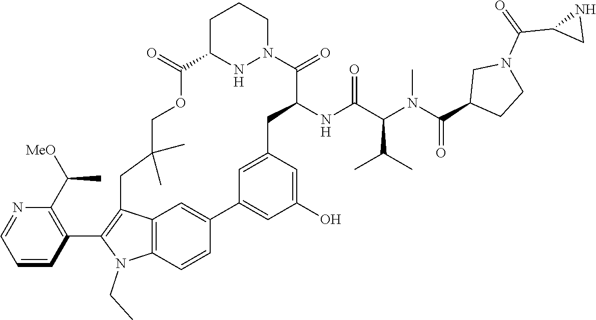 |
| C187 | 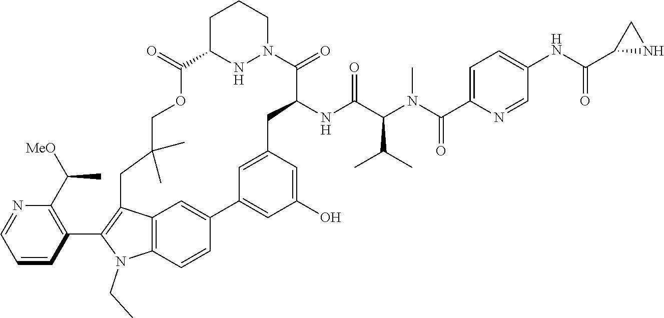 |
| C188 | 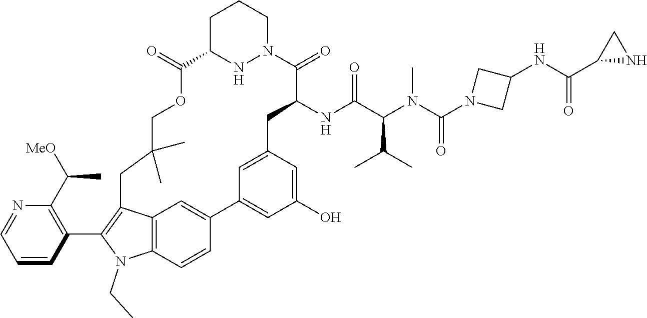 |
| C189 | 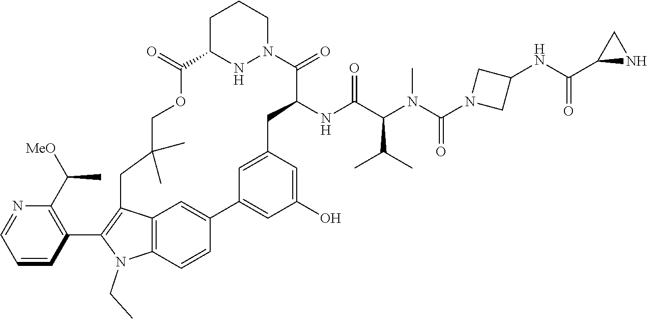 |
| C190 | 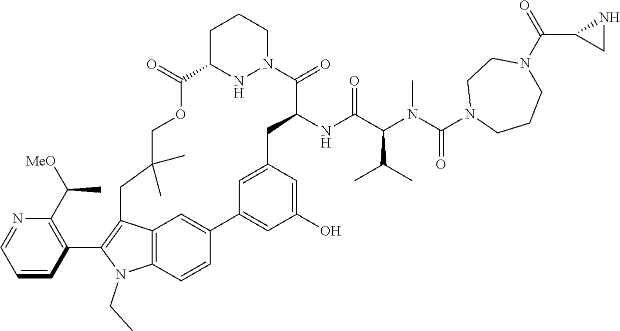 |
| C191 | 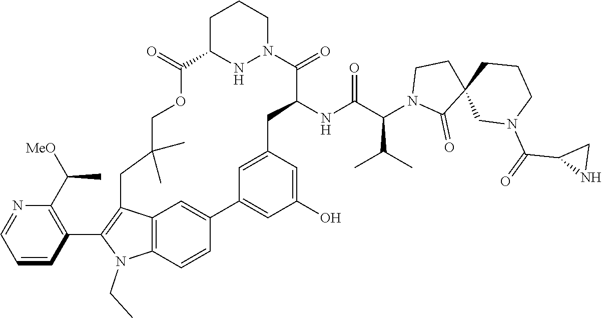 |
| C192 | 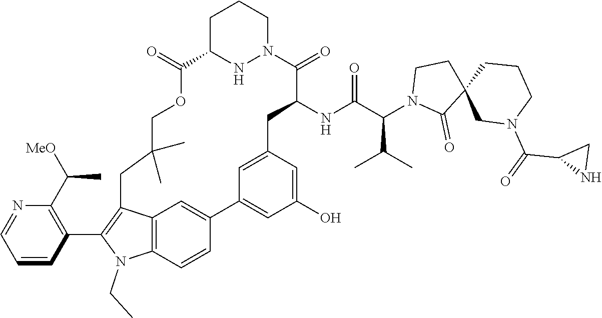 |
| C193 | 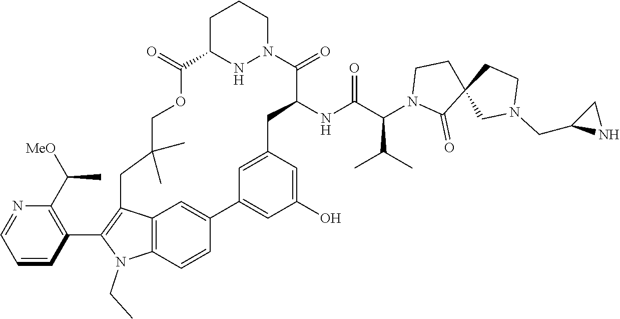 |
| C194 | 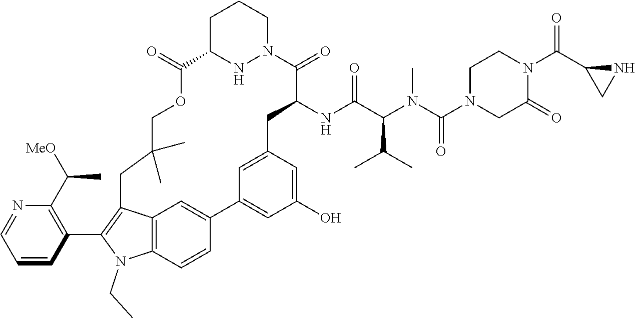 |
| C195 | 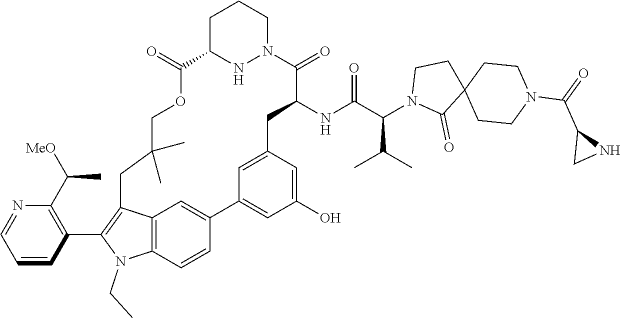 |
| C196 | 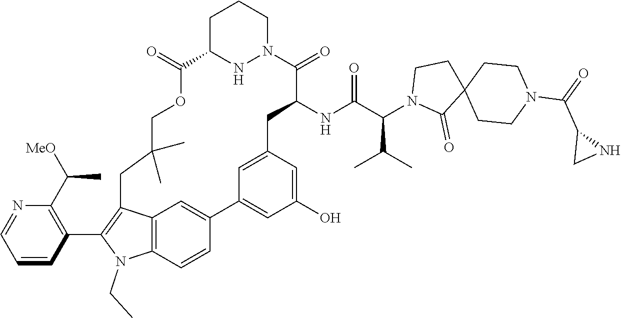 |
| C197 | 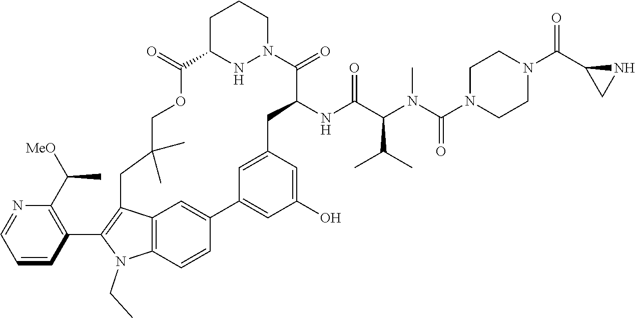 |
| C198 | 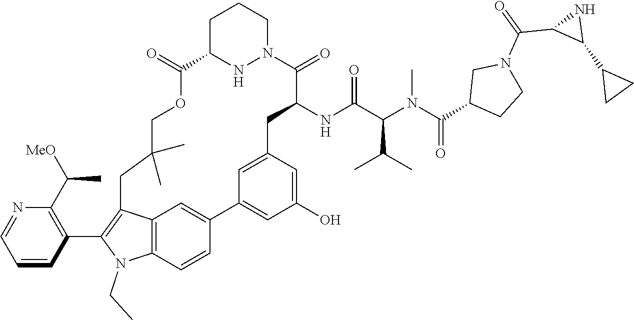 |
| C199 | 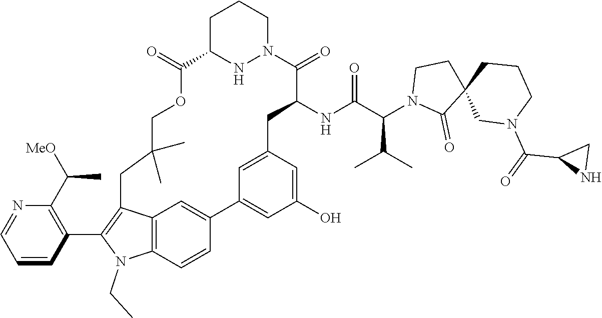 |
| C200 | 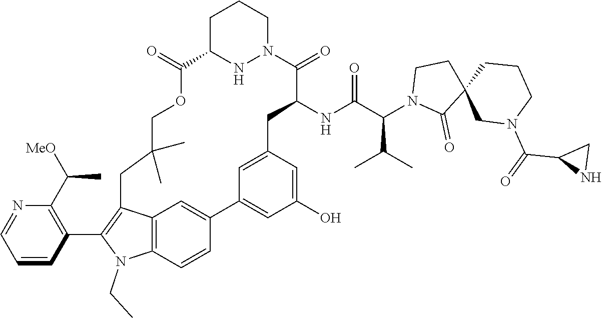 |
| C201 | 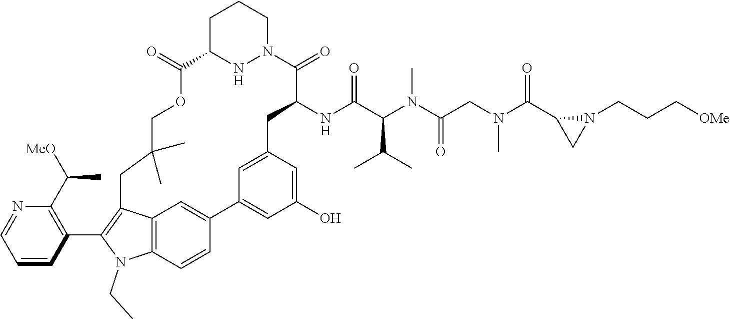 |
| C202 | 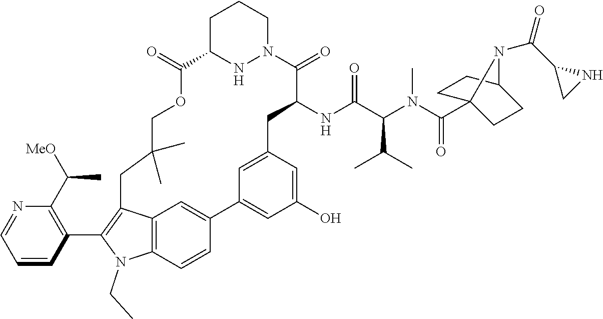 |
| C203 | 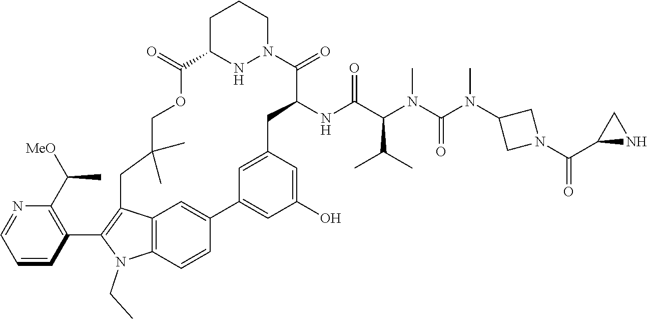 |
| C204 | 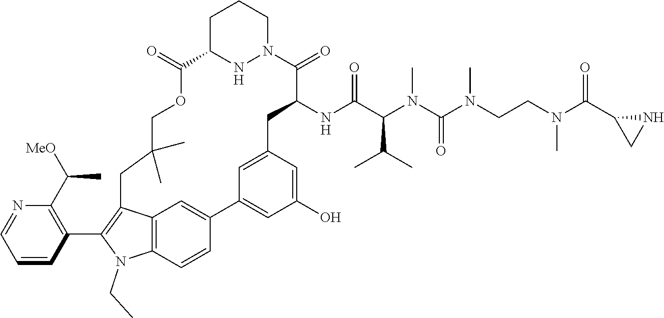 |
| C205 | 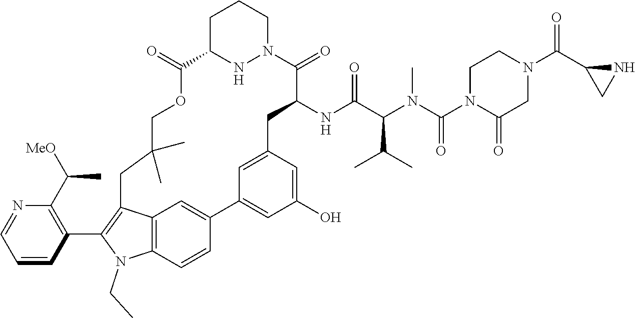 |
| C206 | 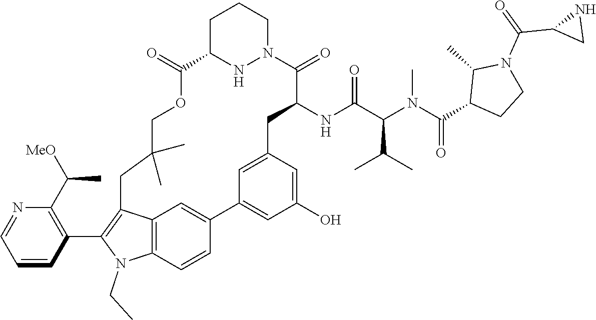 |
| C207 | 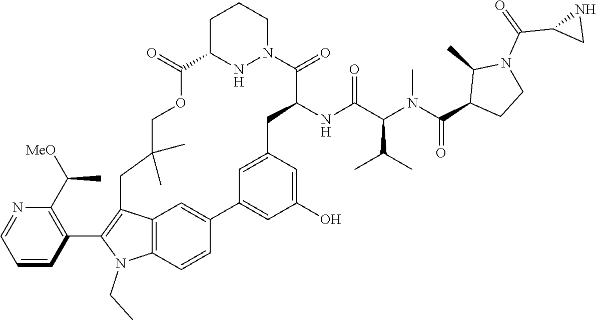 |
| C208 | 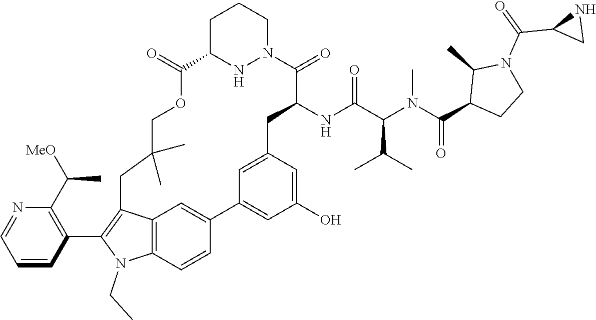 |
| C209 | 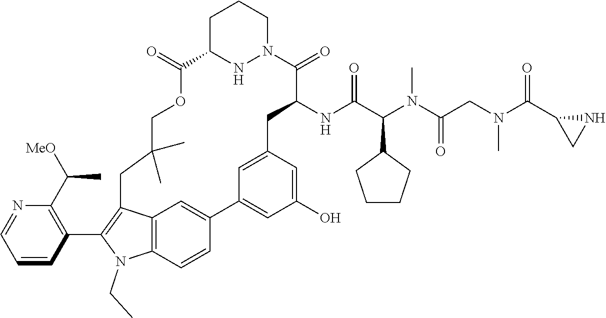 |
| C210 | 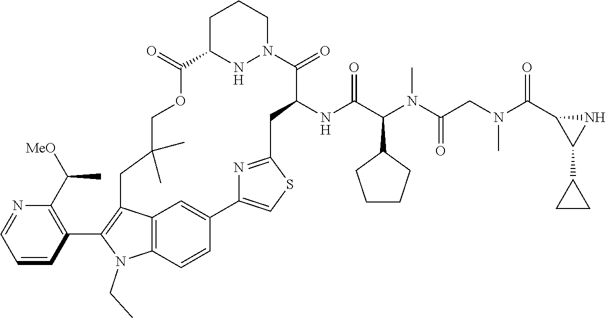 |
| C211 | 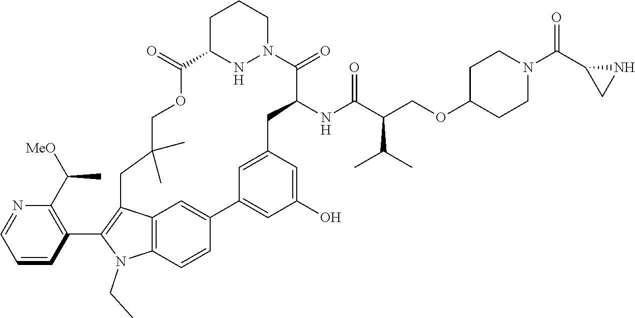 |
| C212 | 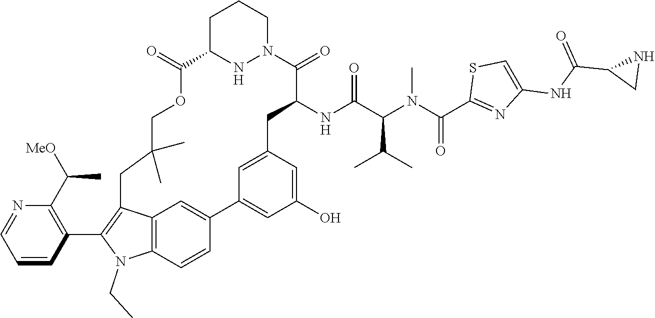 |
| C213 | 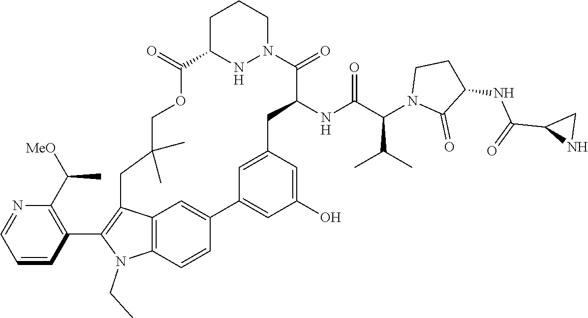 |
| C214 | 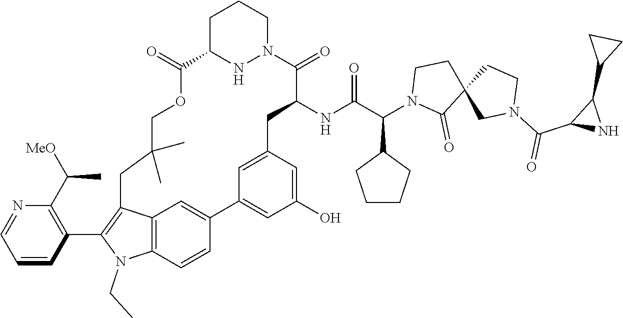 |
| C215 | 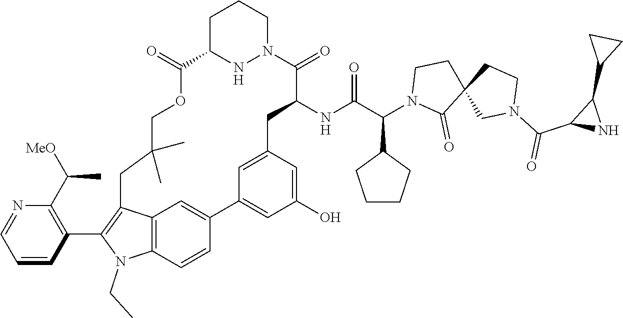 |
| C216 | 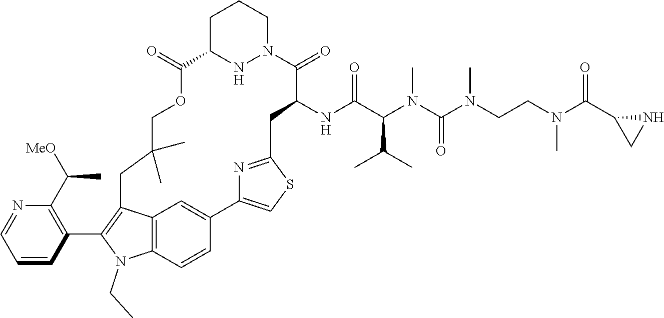 |
| C217 | 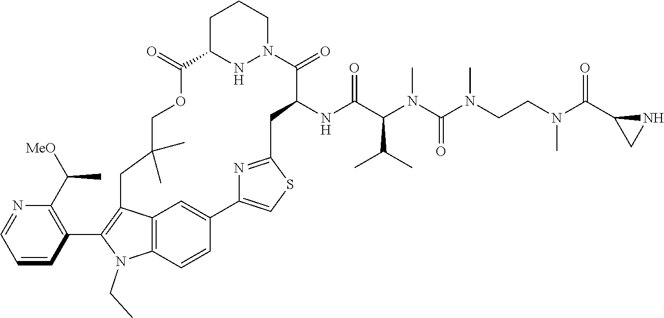 |
| C218 | 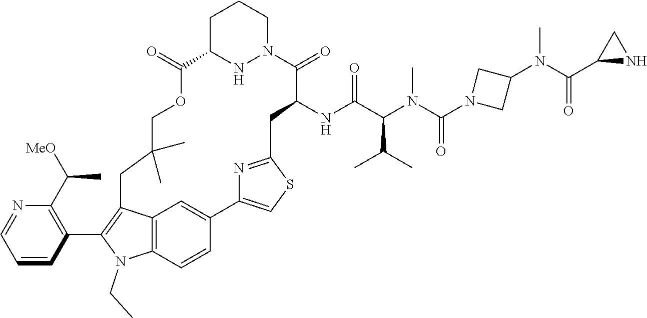 |
| C219 | 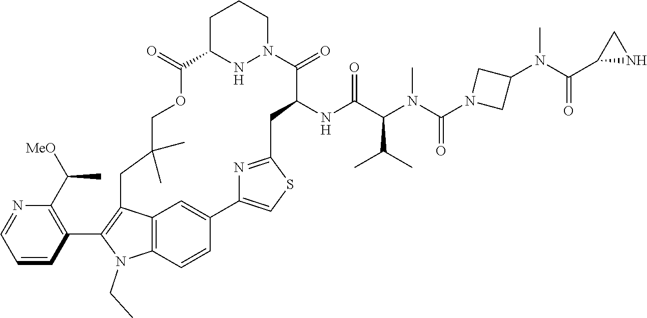 |
| C220 | 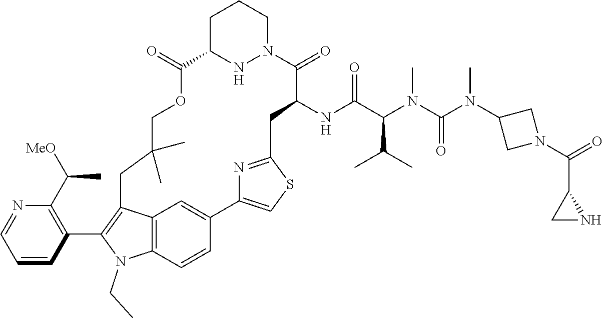 |
| C221 | 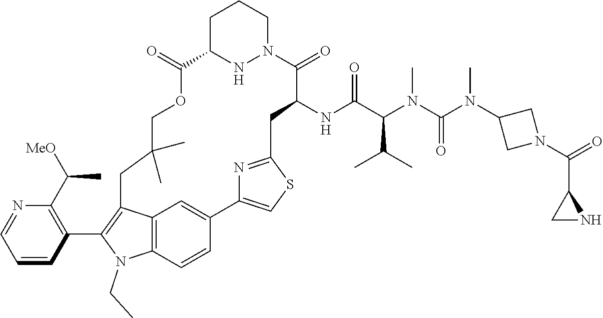 |
| C222 | 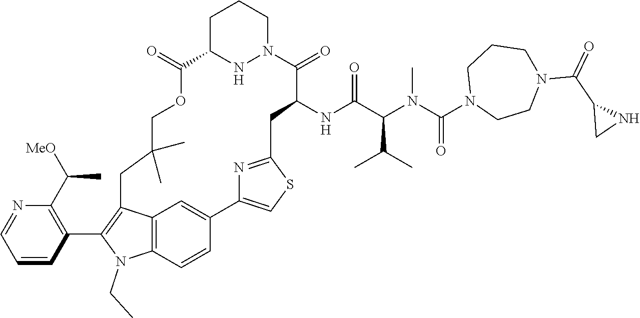 |
| C223 | 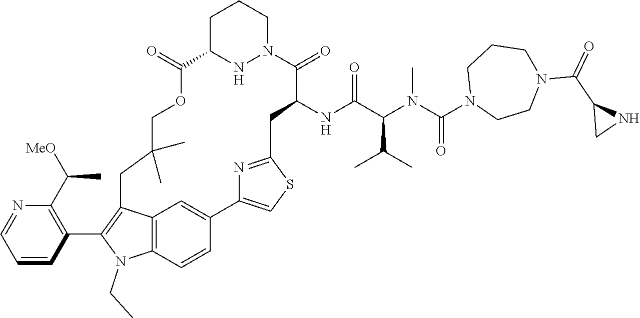 |
| C224 | 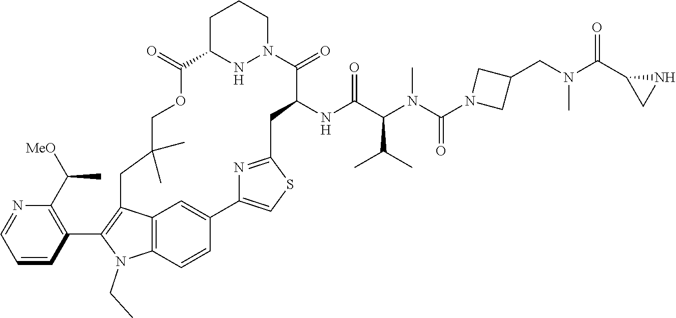 |
| C225 | 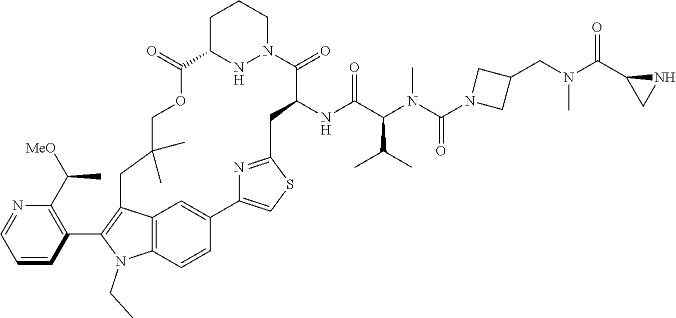 |
| C226 | 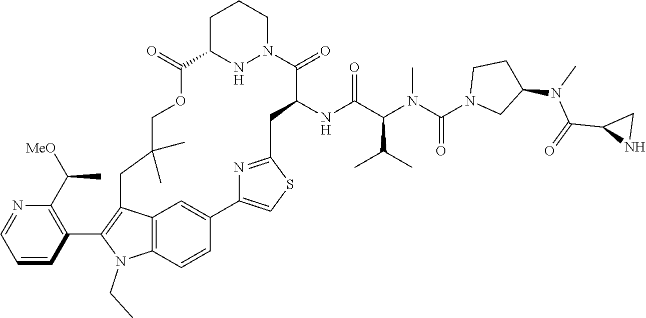 |
| C227 | 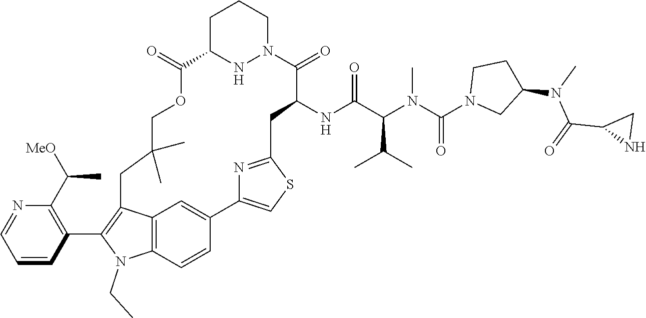 |
| C228 | 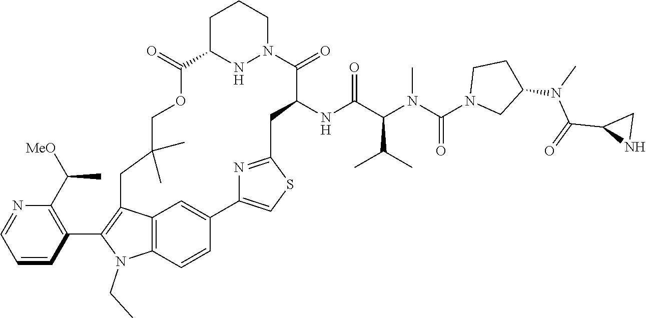 |
| C229 | 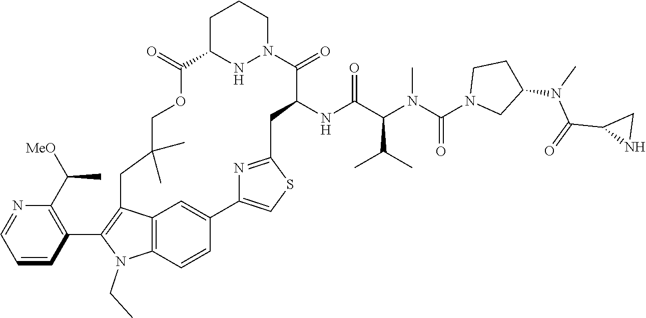 |
| C230 | 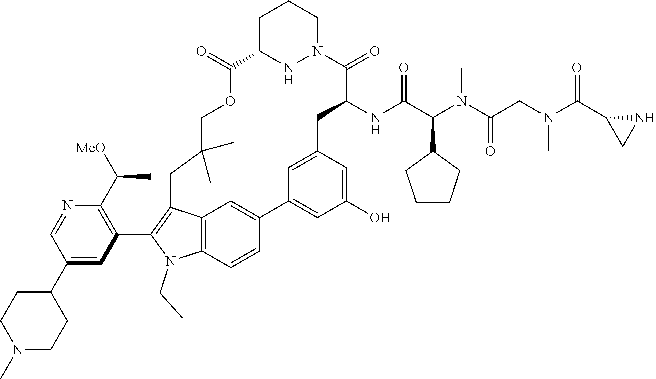 |
| C231 | 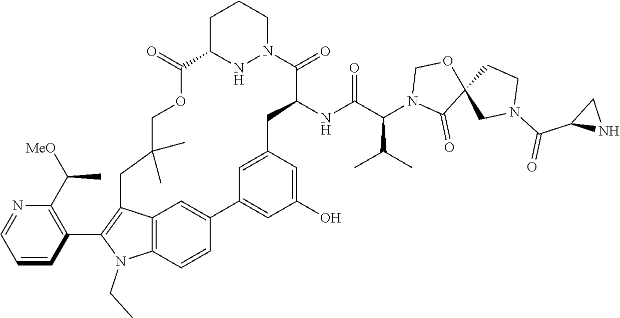 |
| C232 | 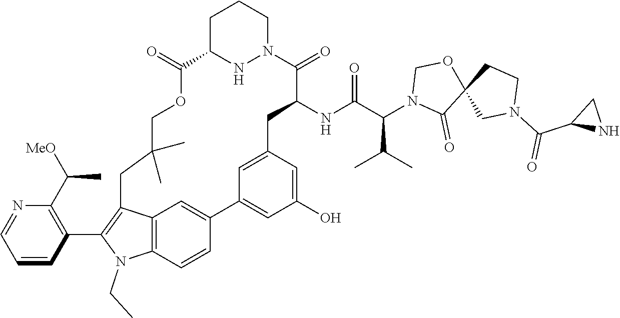 |
| C233 | 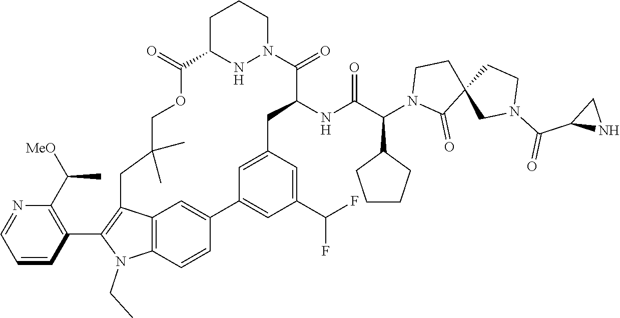 |
| C234 | 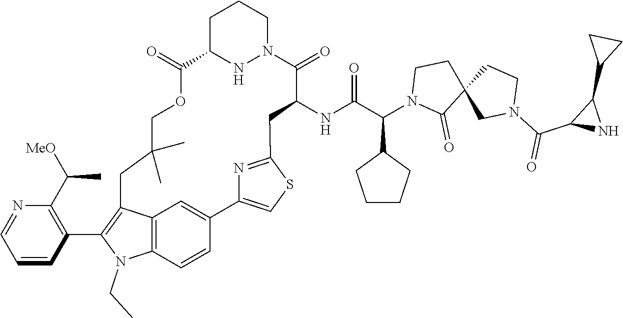 |
| C235 | 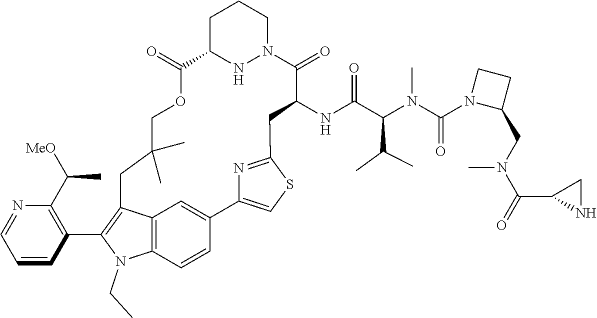 |
| C236 | 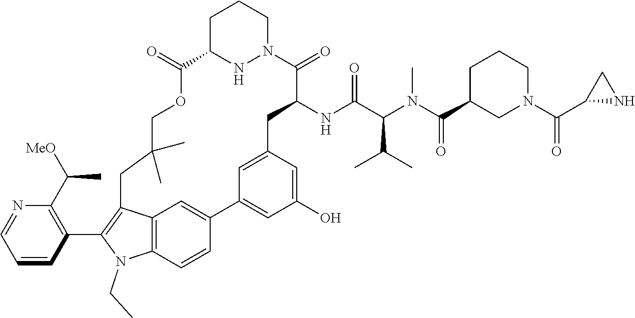 |
| C237 | 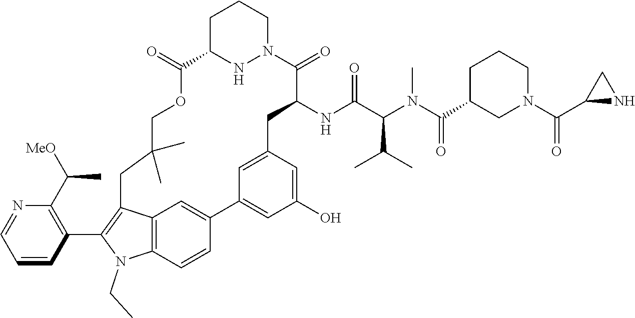 |
| C238 | 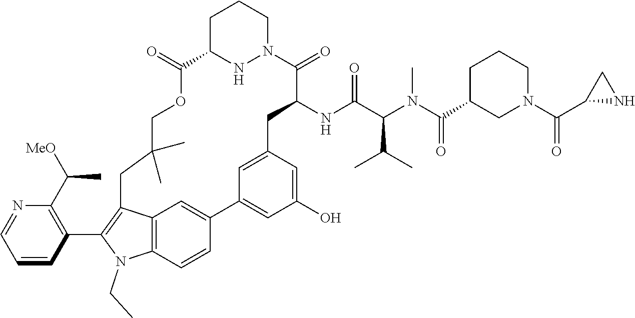 |
| C239 | 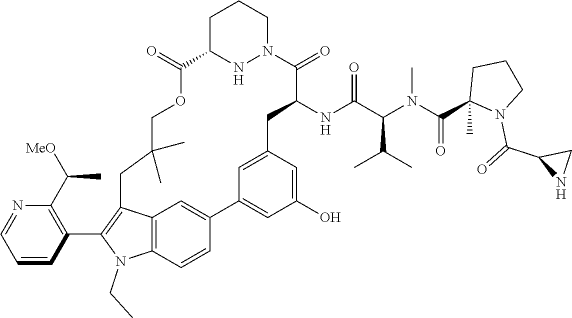 |
| C240 | 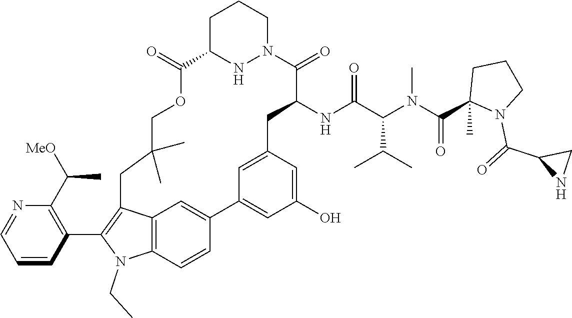 |
| C241 | 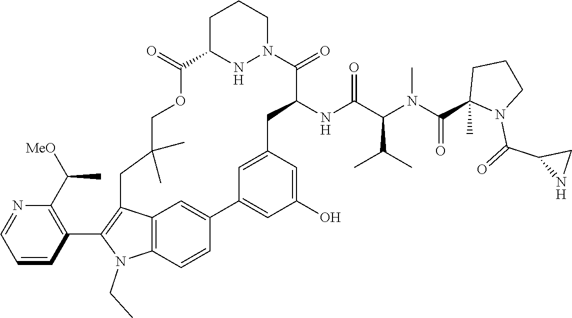 |
| C242 | 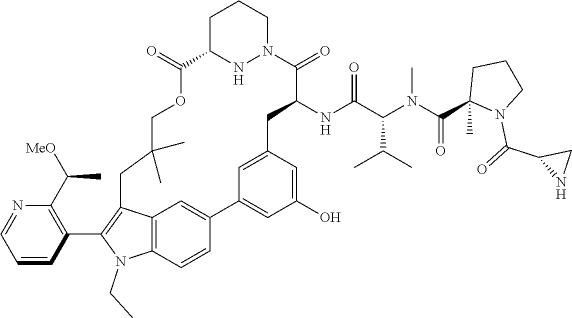 |
| C243 | 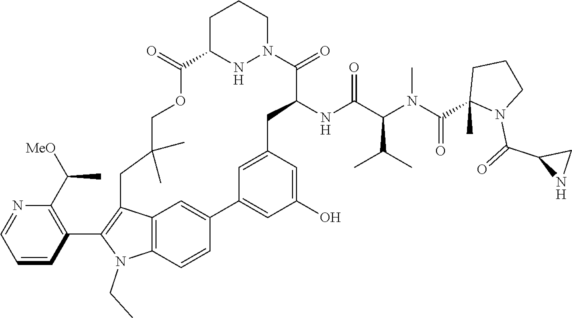 |
| C244 | 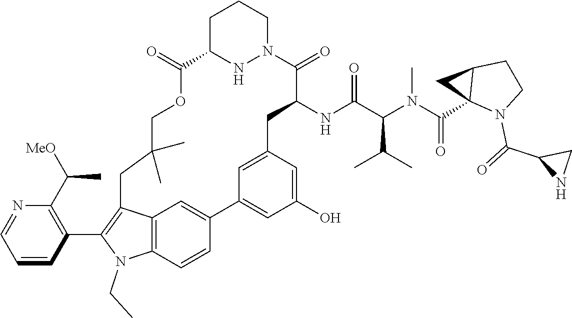 |
| C245 | 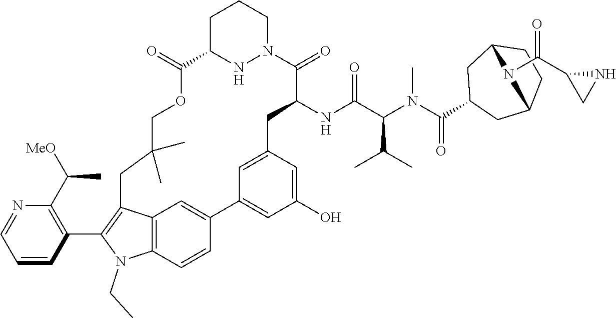 |
| C246 | 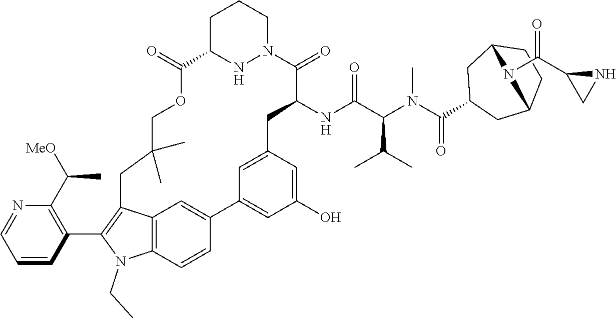 |
| C247 | 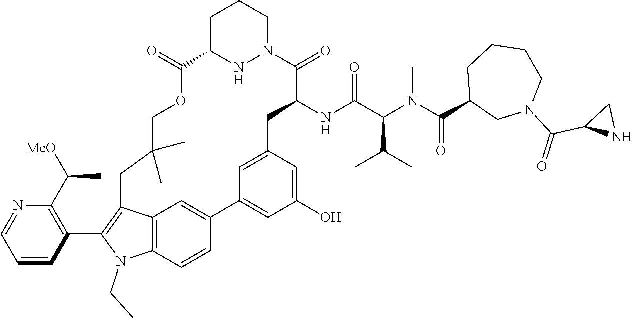 |
| C248 | 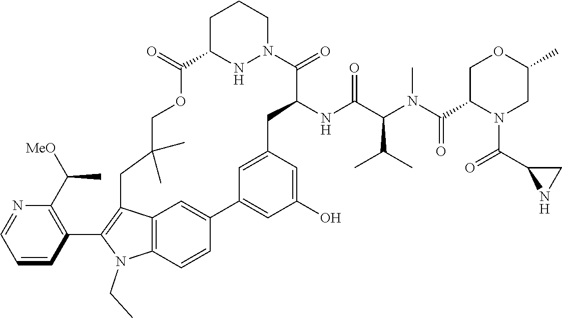 |
| C249 | 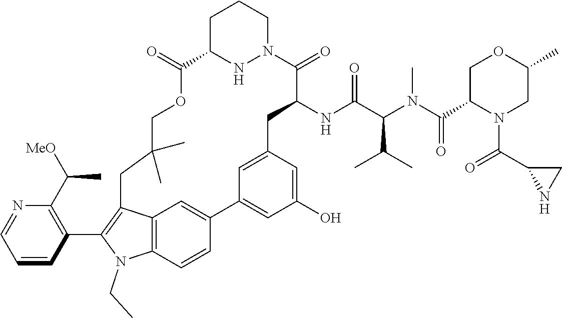 |
| C250 | 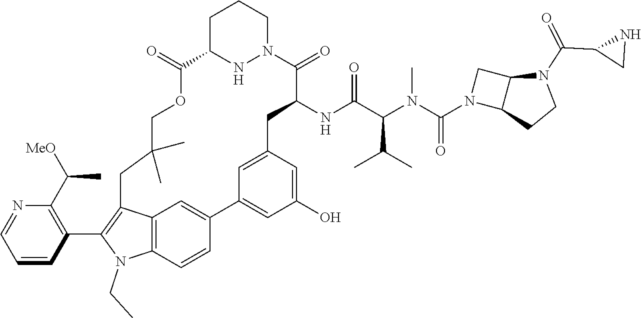 |
| C251 | 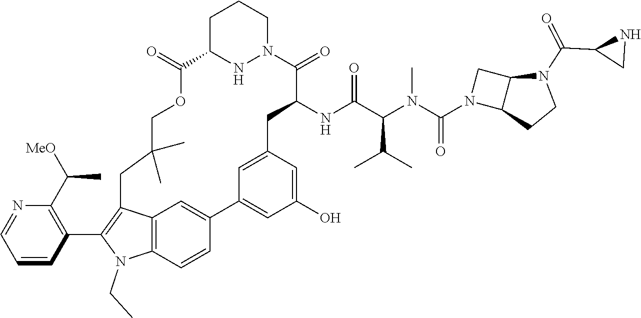 |
| C252 | 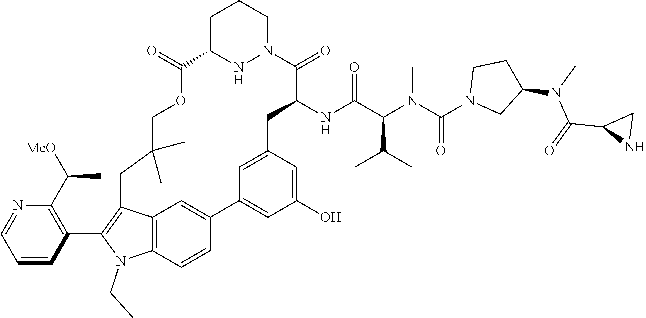 |
| C253 | 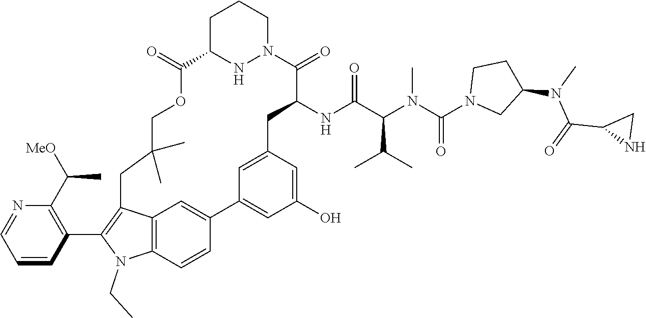 |
| C254 | 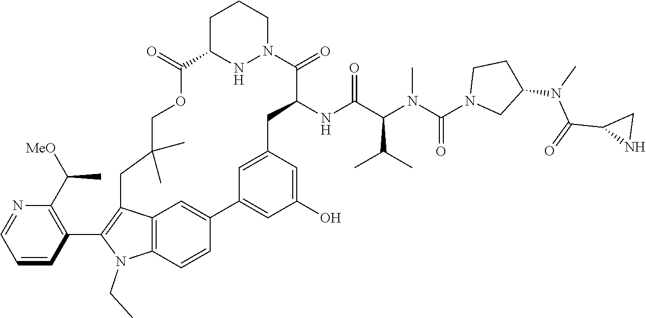 |
| C255 | 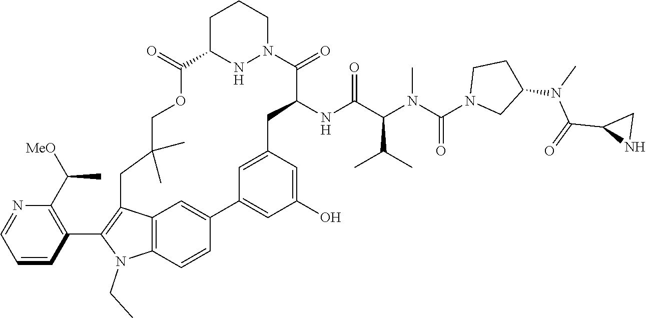 |
| C256 | 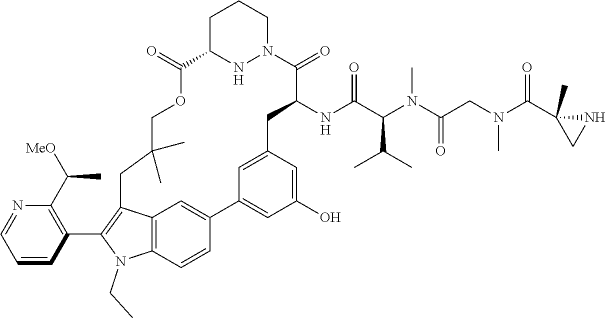 |
| C257 | 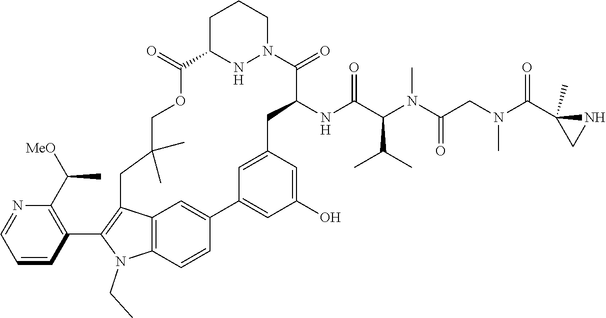 |
| C258* | 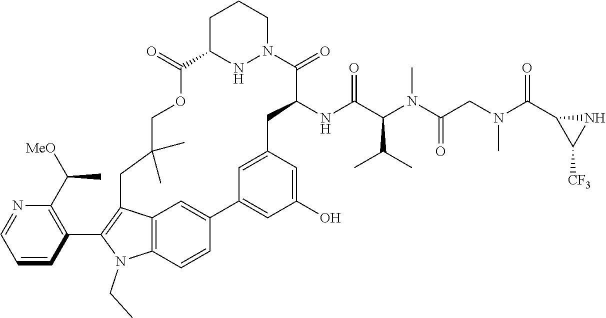 |
| C259* | 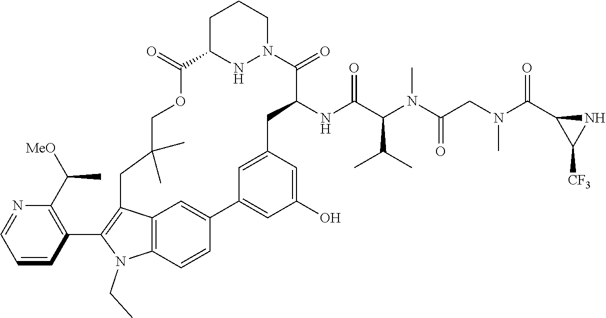 |
| C260* | 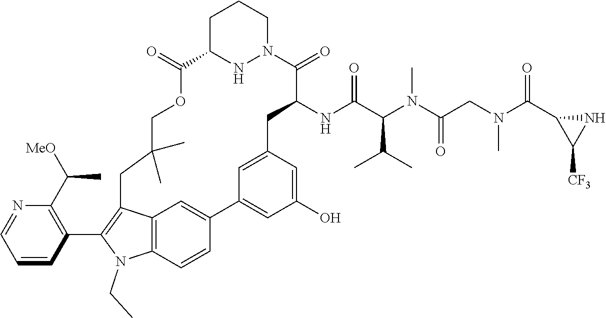 |
| C261* | 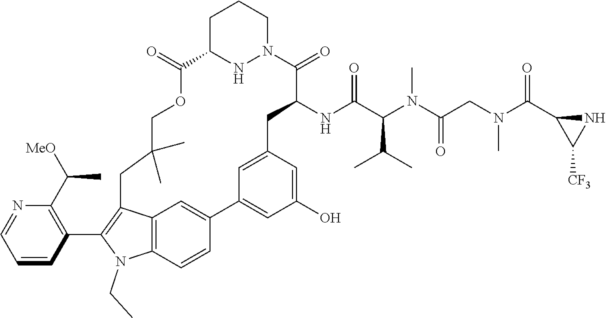 |
| C262 | 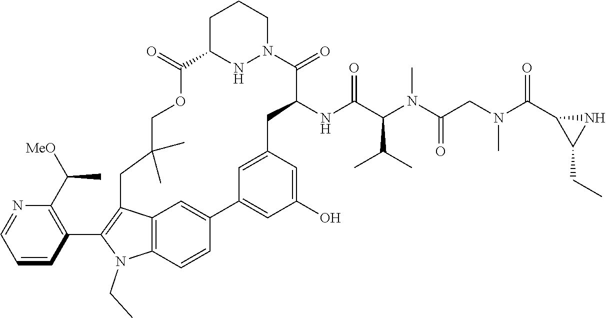 |
| C263 | 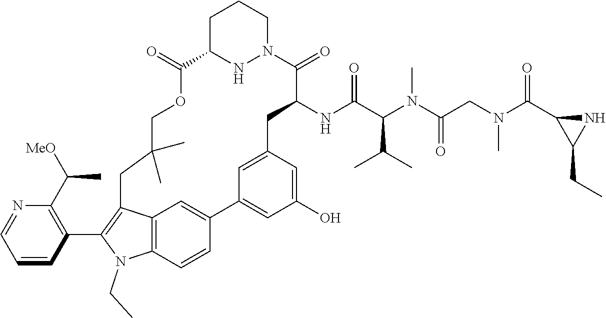 |
| C264 | 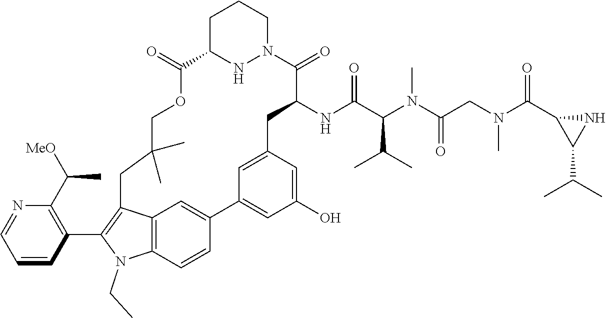 |
| C265 | 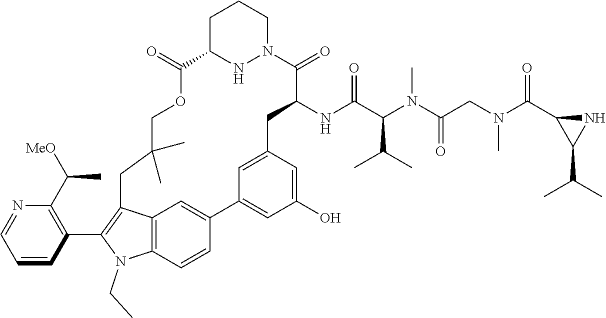 |
| C266 | 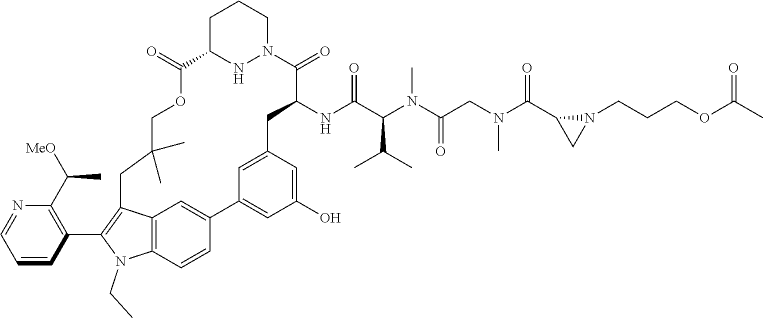 |
| C267 | 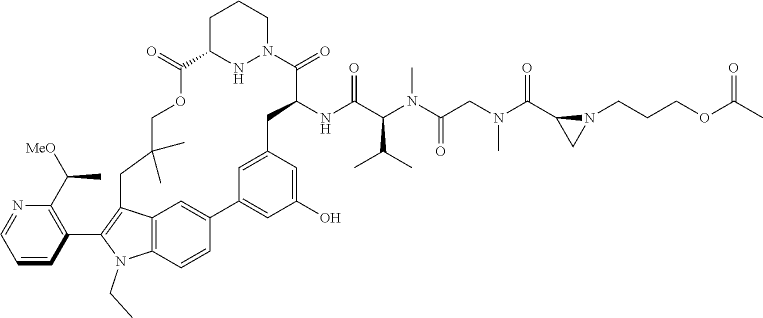 |
| C268 | 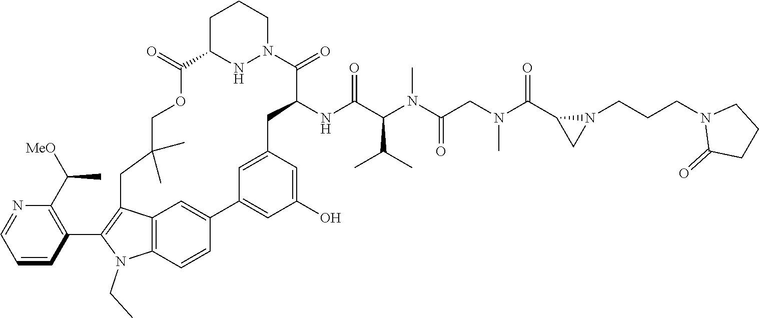 |
| C269 | 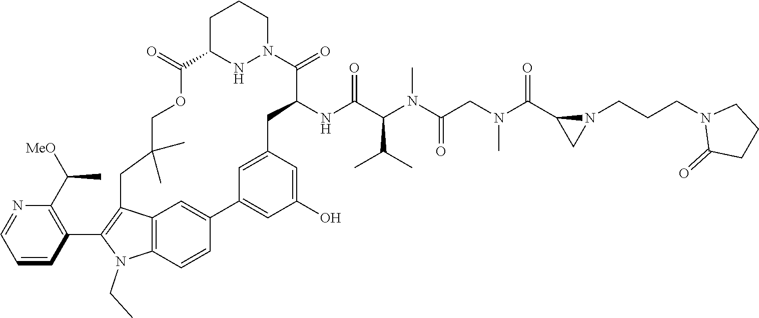 |
| C270 | 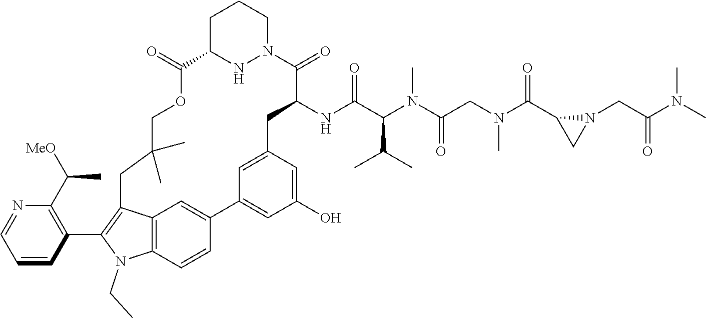 |
| C271 | 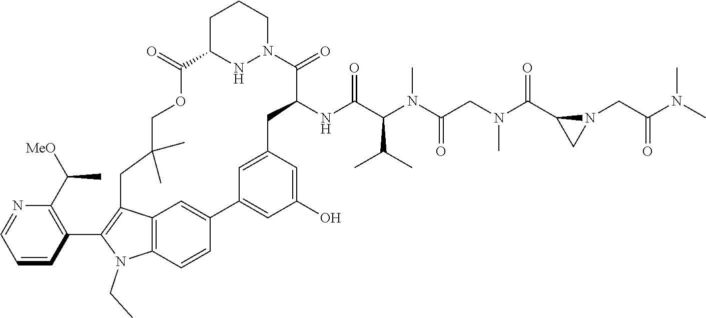 |
| C272 | 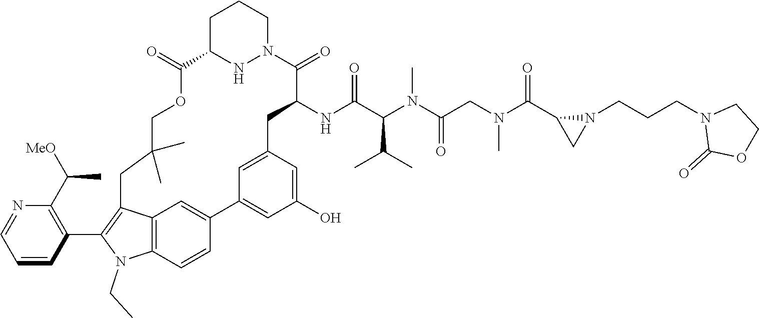 |
| C273 | 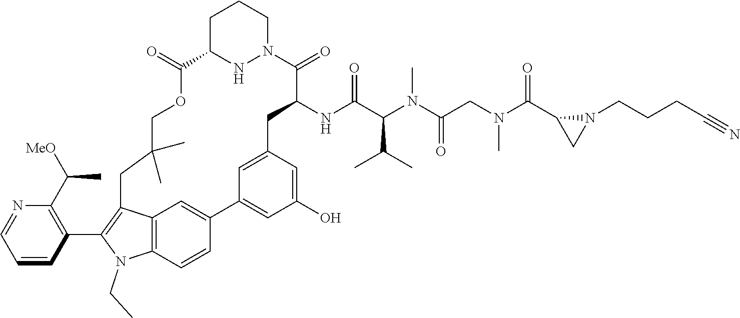 |
| C274 | 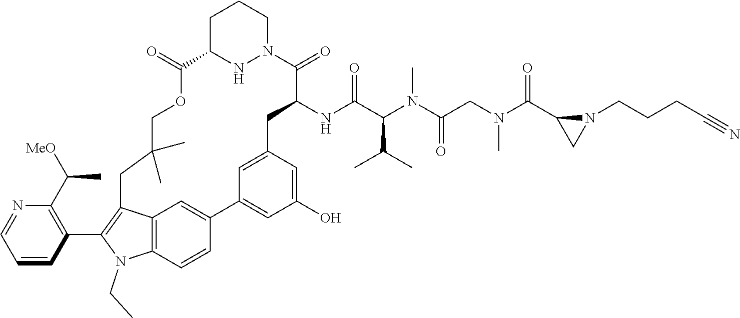 |
| C275 | 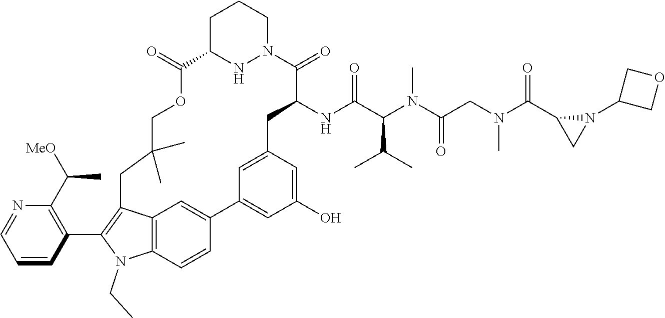 |
| C276 | 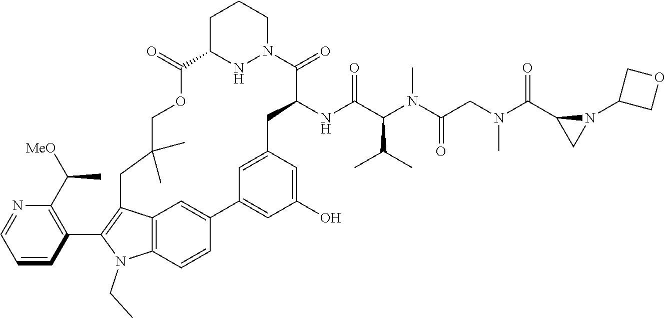 |
| C277 | 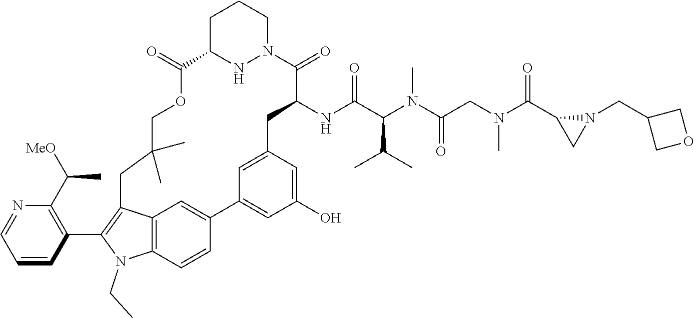 |
| C278 | 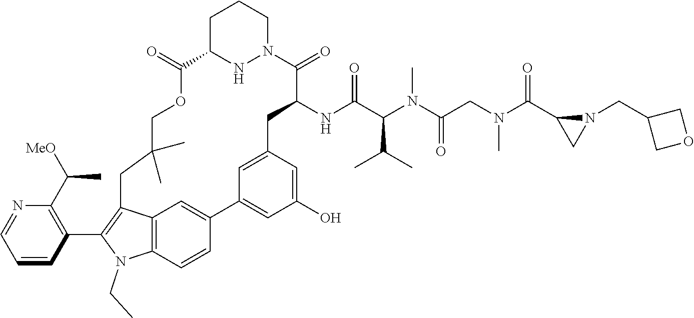 |
| C279 | 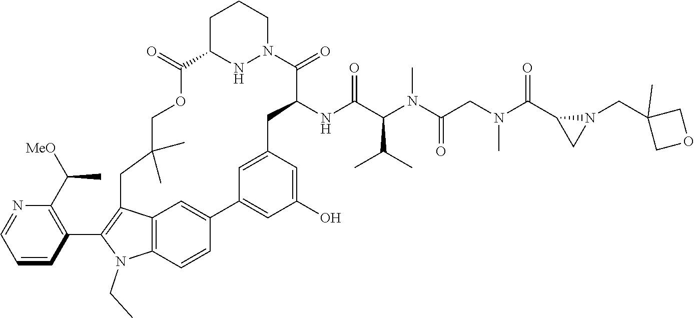 |
| C280 | 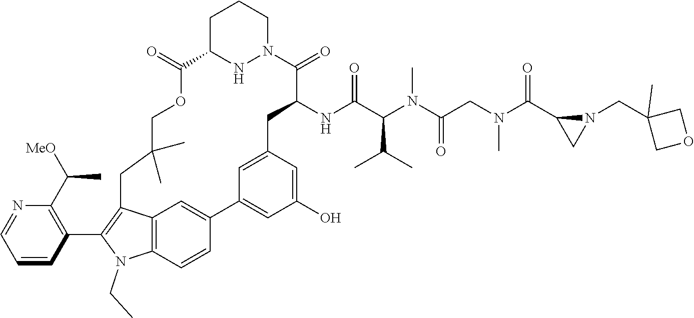 |
| C281* | 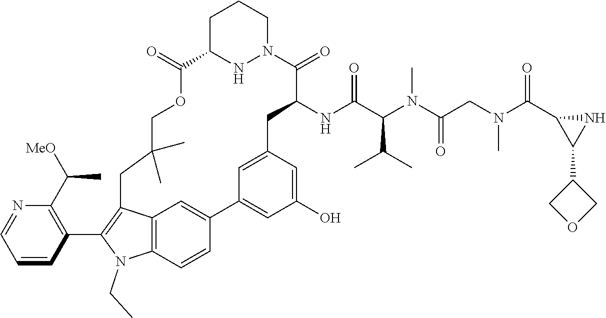 |
| C282* | 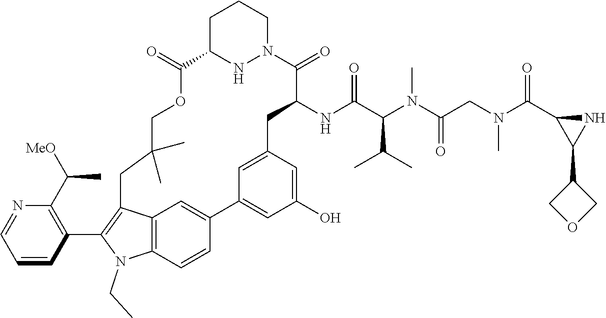 |
| C283* | 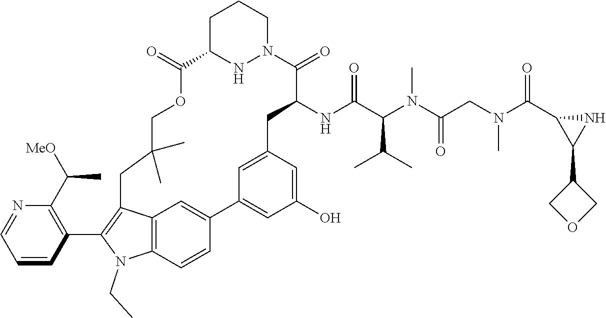 |
| C284* | 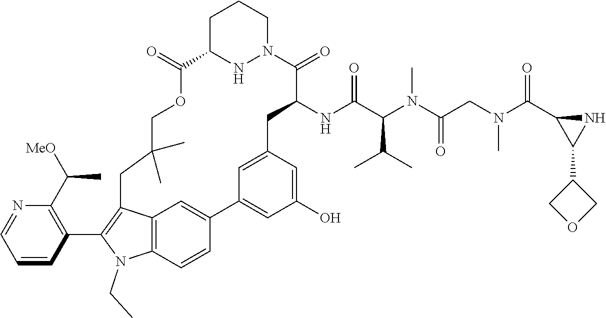 |
| C285 | 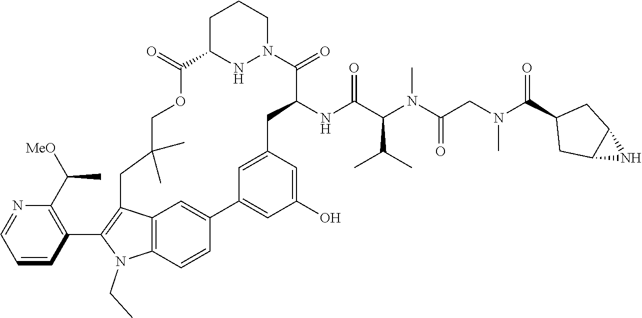 |
| C286 | 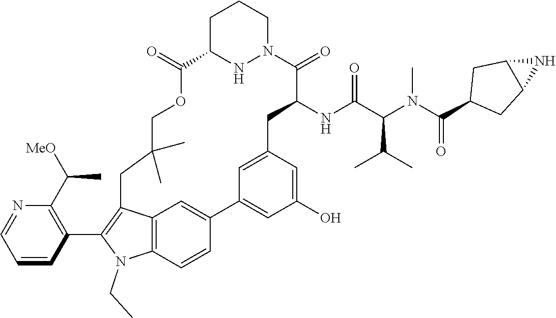 |
| C287 | 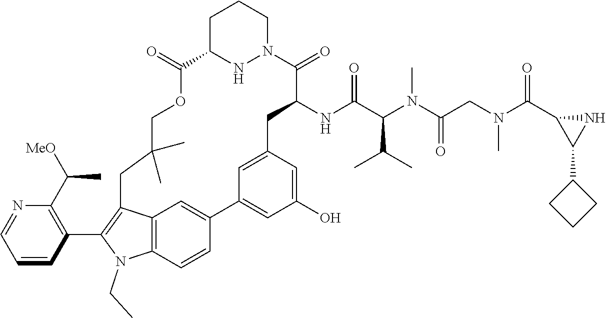 |
| C288 | 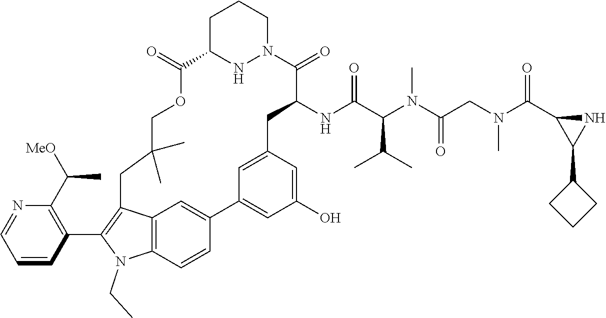 |
| C289 | 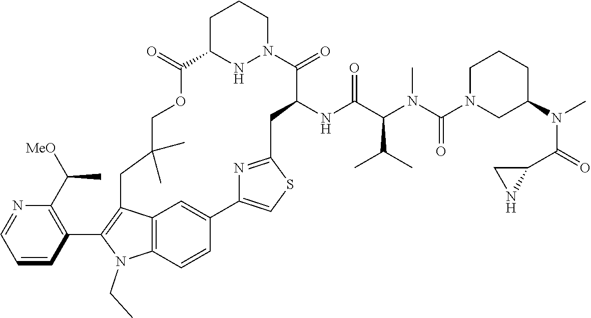 |
| C290 | 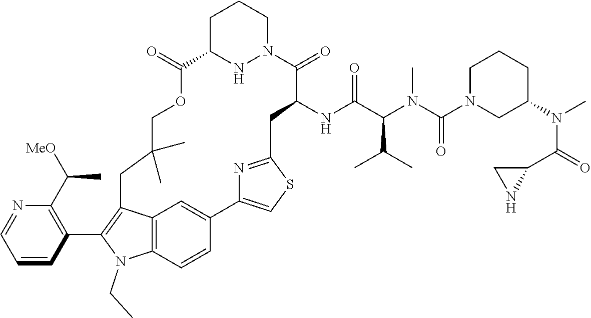 |
| C291 | 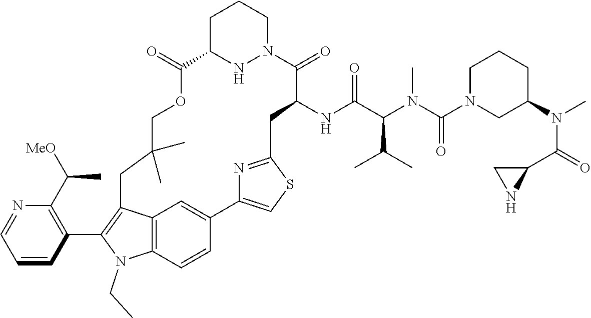 |
| C292 | 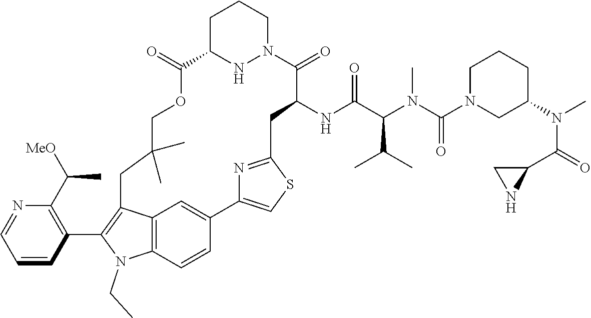 |
| C293 | 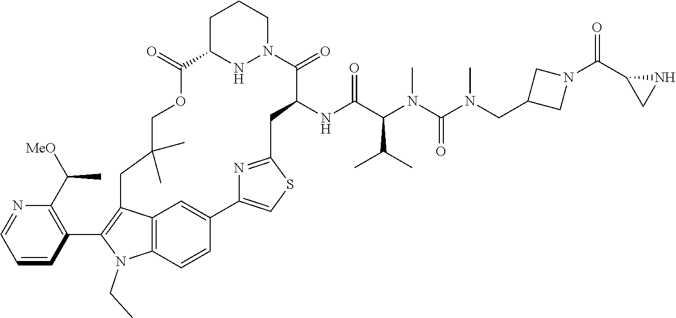 |
| C294 | 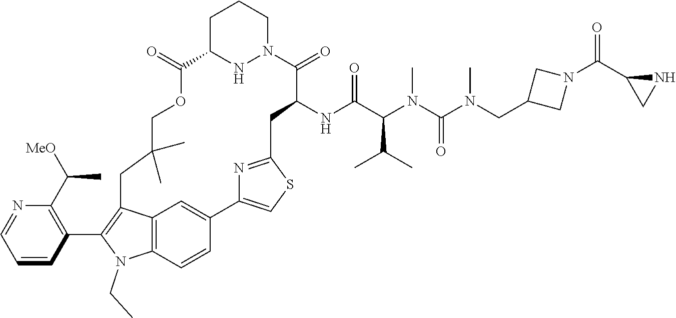 |
| C295 | 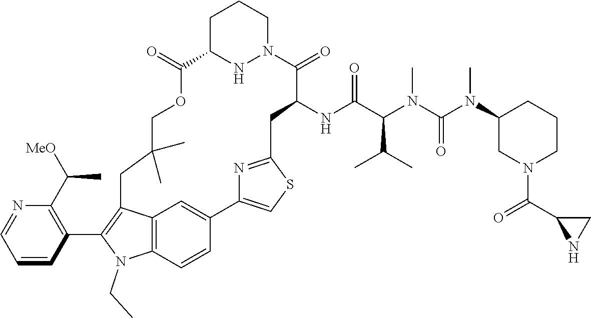 |
| C296 | 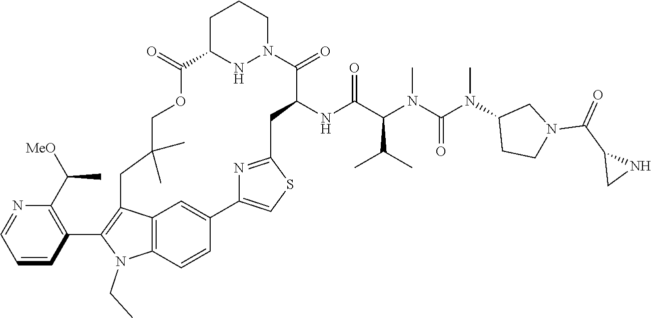 |
| C297 | 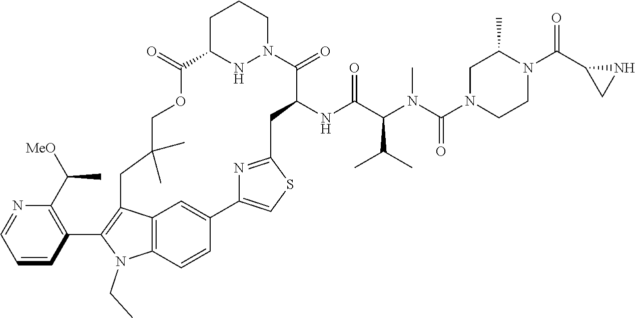 |
| C298 | 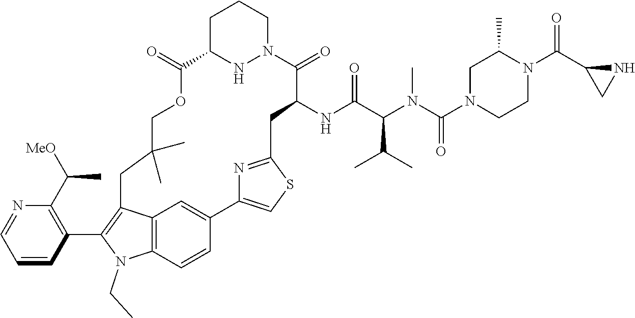 |
| C299 | 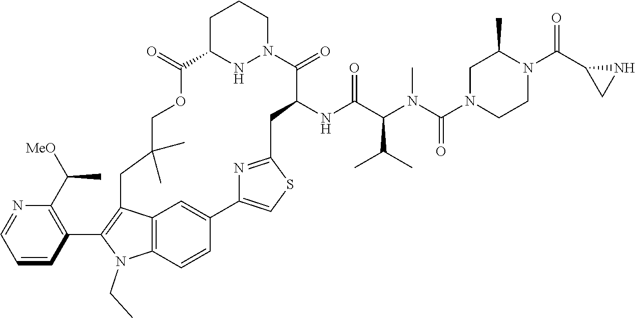 |
| C300 | 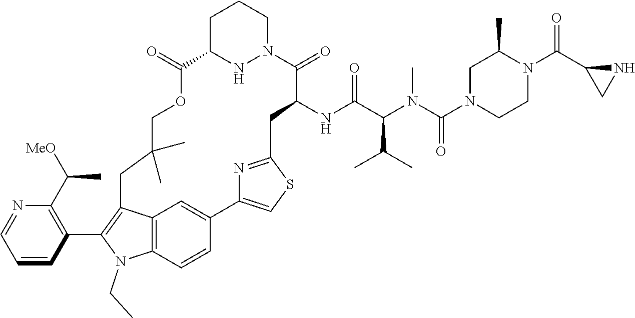 |
| C301 | 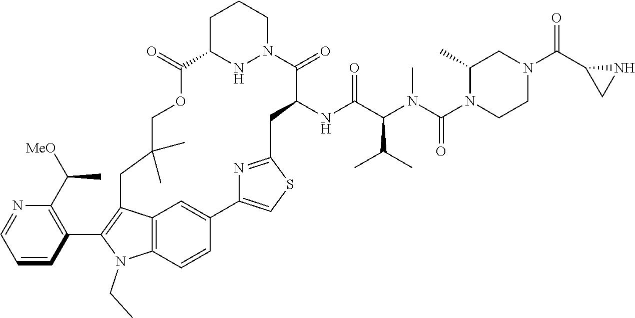 |
| C302 | 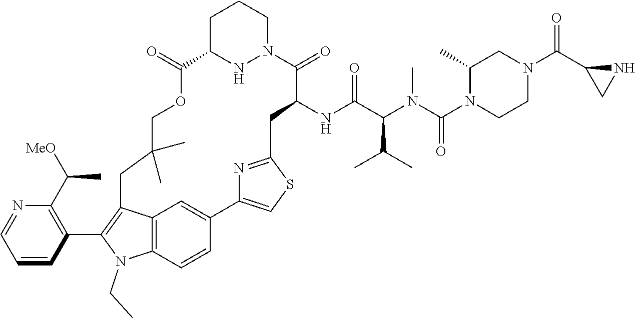 |
| C303 | 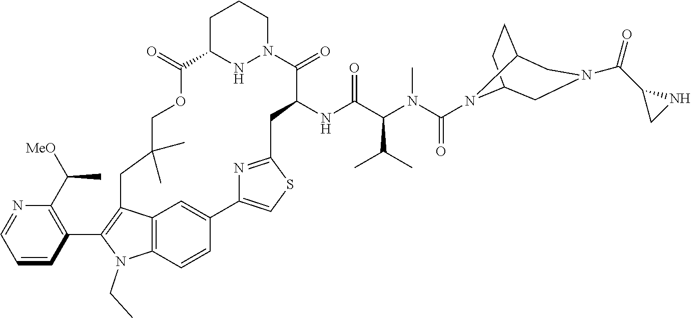 |
| C304 | 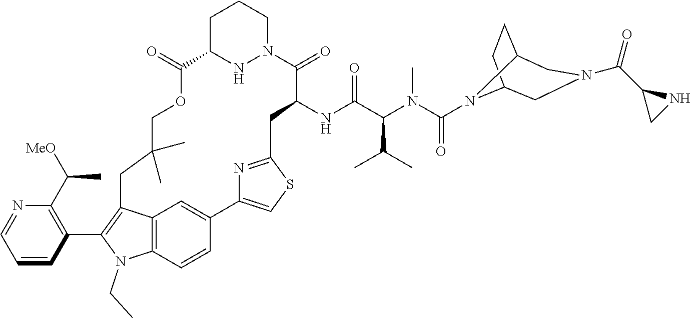 |
| C305 | 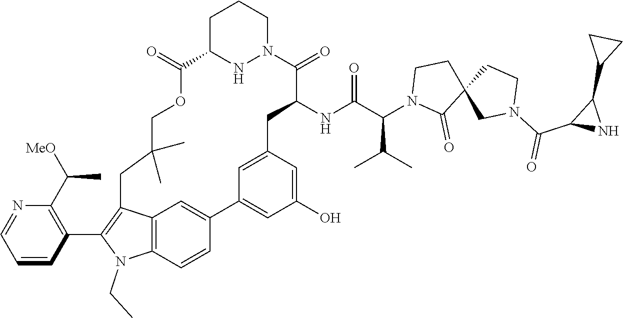 |
| C306 | 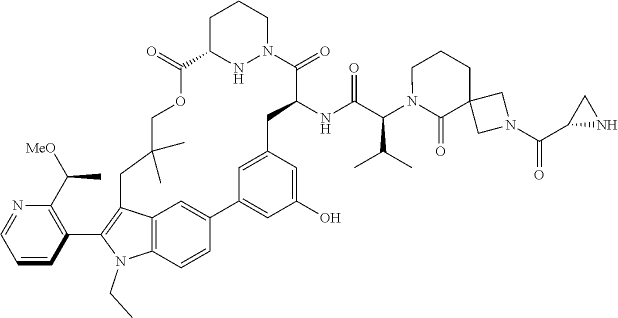 |
| C307 | 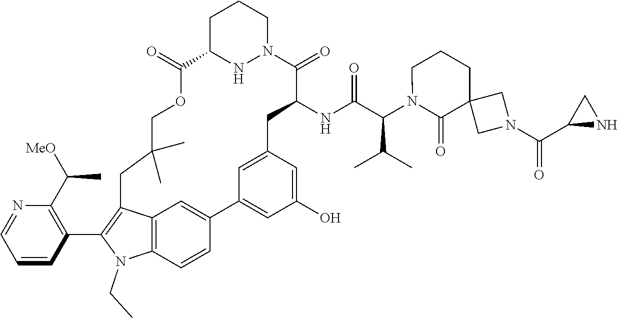 |
| C308* | 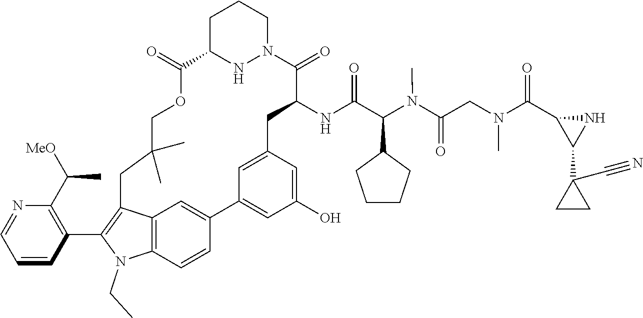 |
| C309 | 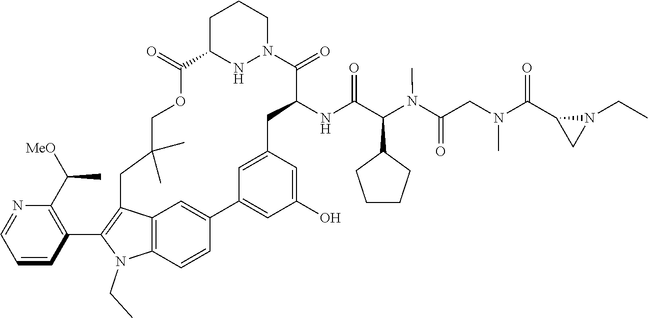 |
| C310 | 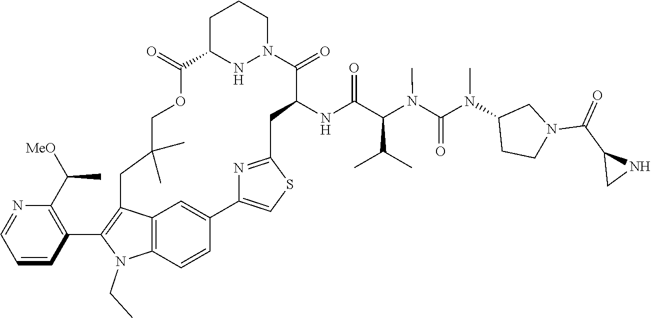 |
| C311 | 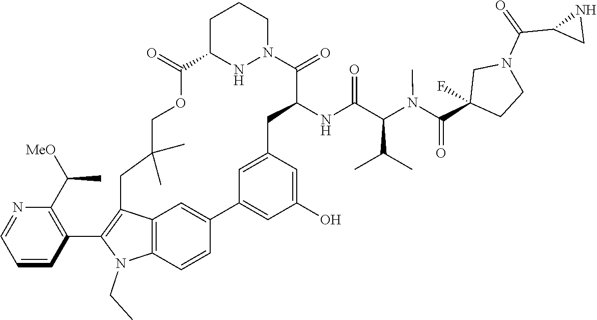 |
| C312 | 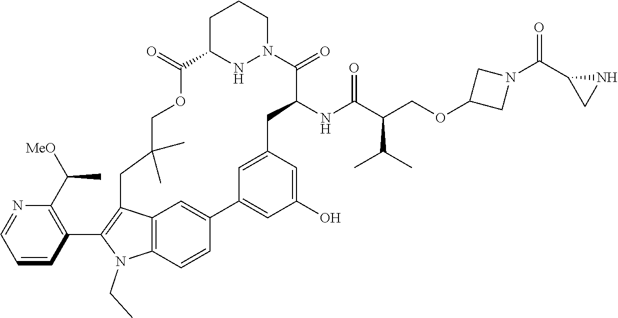 |
| C313 | 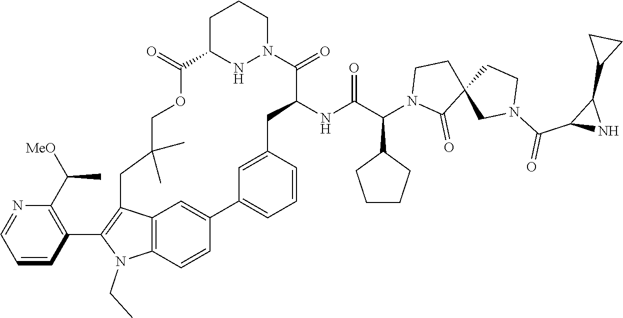 |
| C314* | 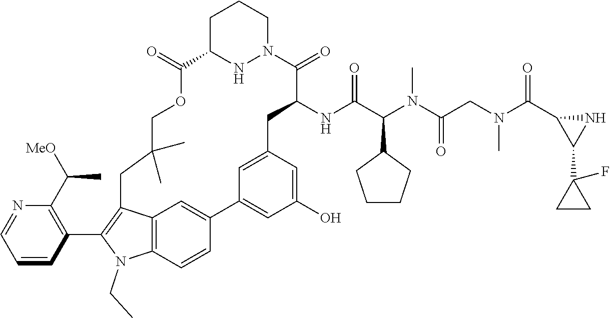 |
| C315* | 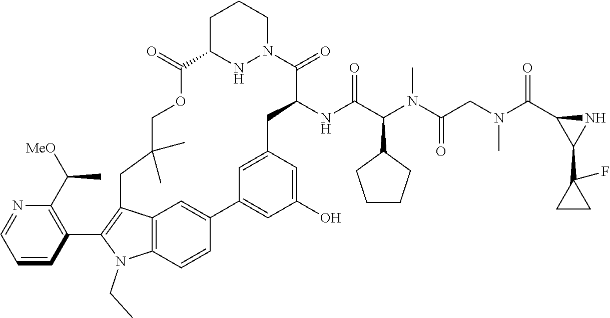 |
| C316* | 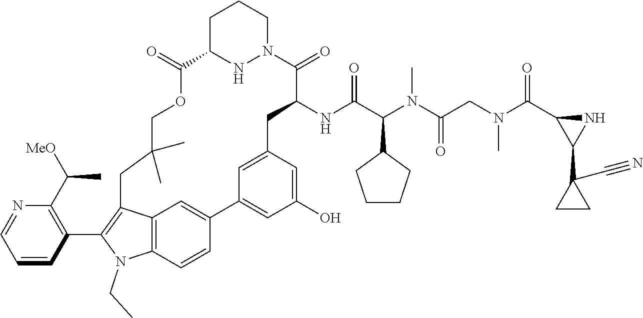 |
| C317 | 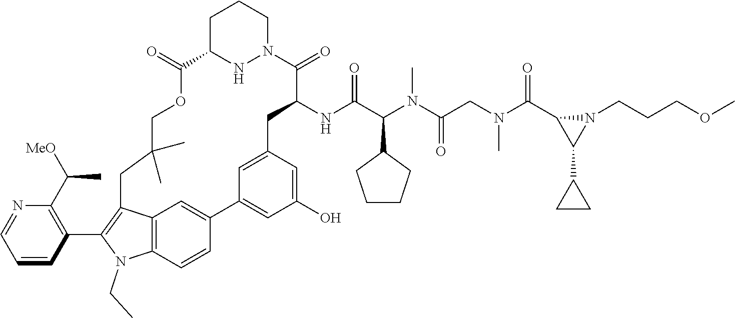 |
| C318 | 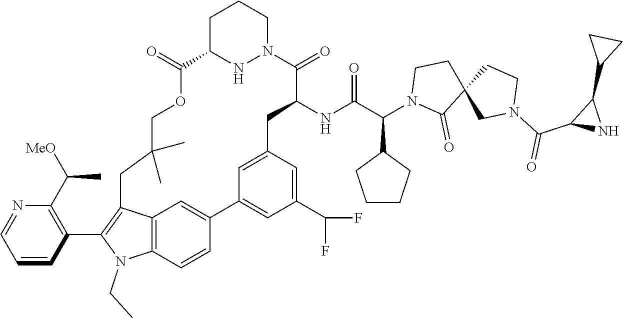 |
| C319 | 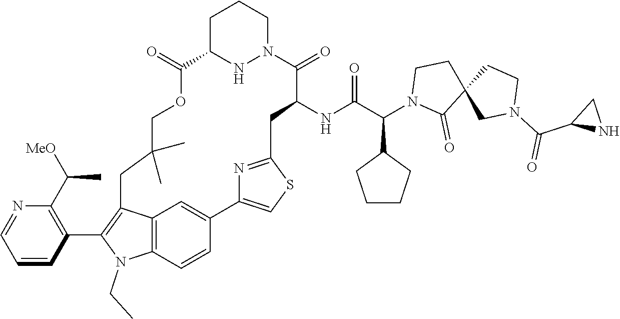 |
| C320 | 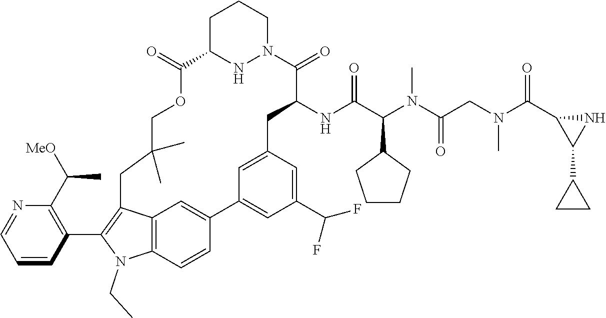 |
| C321 | 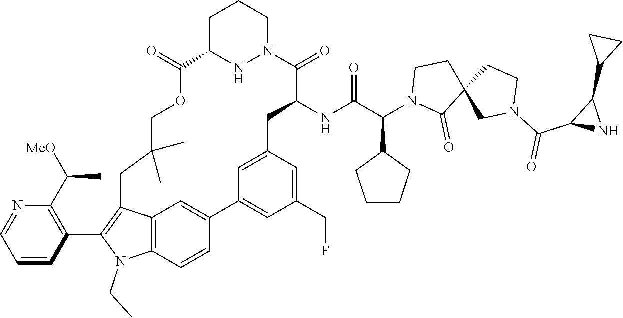 |
| C322* | 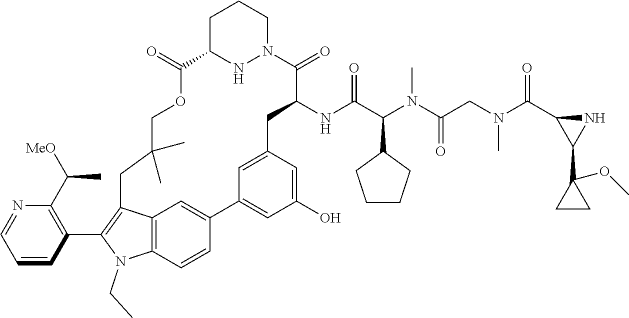 |
| C323* | 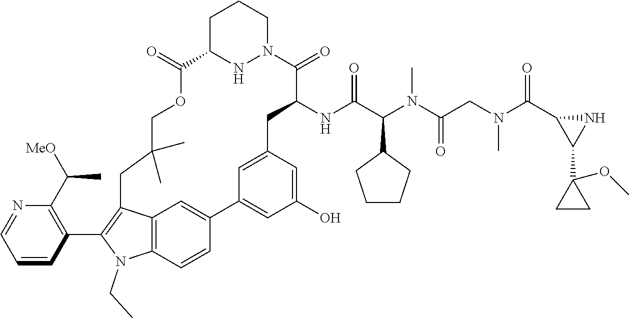 |
| C324 | 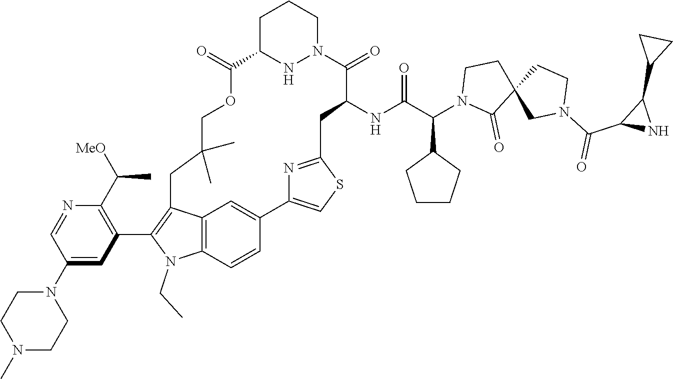 |
| C325 | 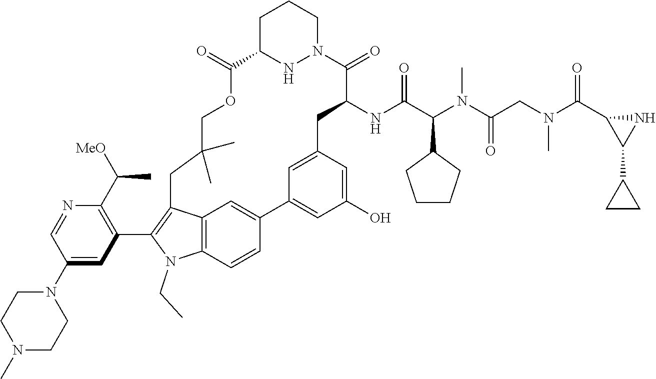 |
| C326 | 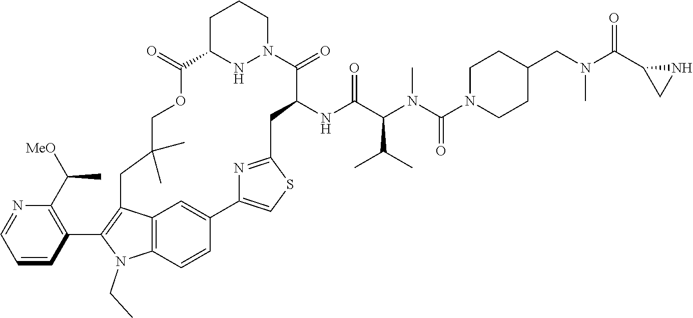 |
| C327 | 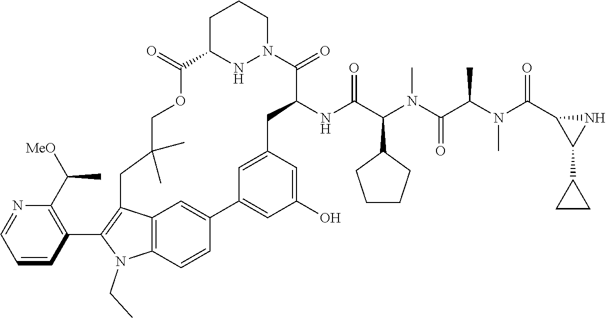 |
| C328 | 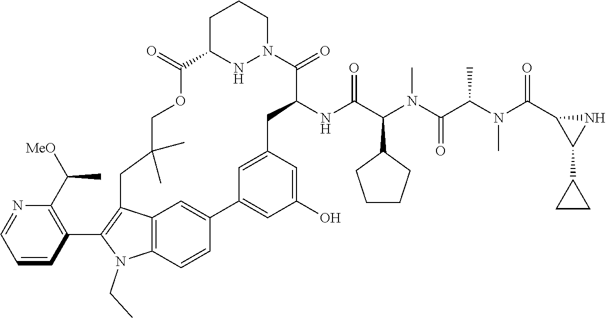 |
| C329 | 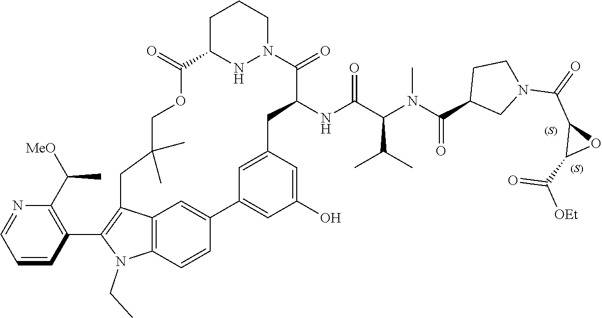 |
| C330 | 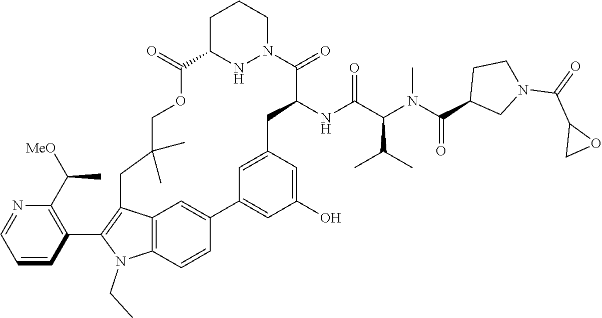 |
| C331 | 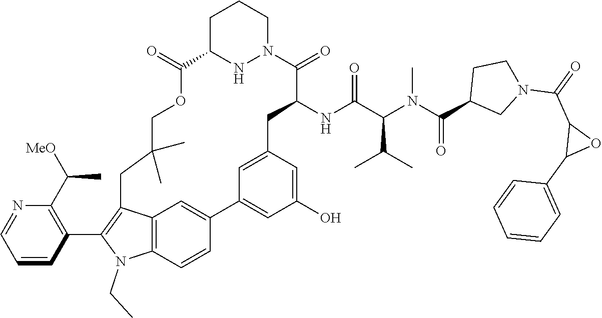 |
| C332 | 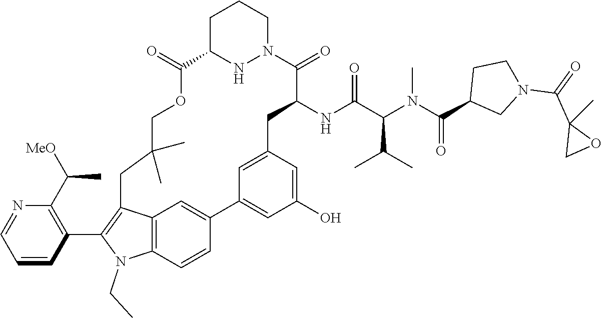 |

*Stereochemistry of the aziridine carbon is assumed.
Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.

In some embodiments, a compound of Table C2 is provided, or a pharmaceutically acceptable salt thereof. In some embodiments, the RAS(ON) inhibitor is selected from Table C2, or a pharmaceutically acceptable salt or atropisomer thereof.

TABLE C2
Certain Compounds of the Present Invention

| Ex# | Structure |
|---|---|
| CB5 | 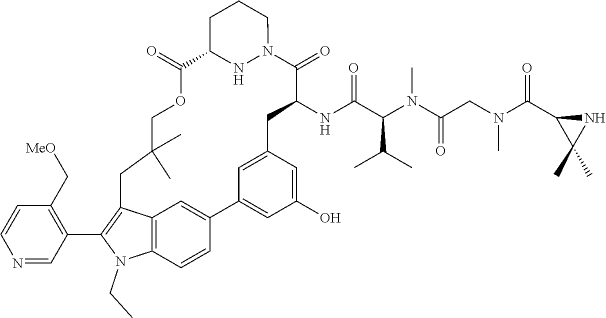 |
| CB6 | 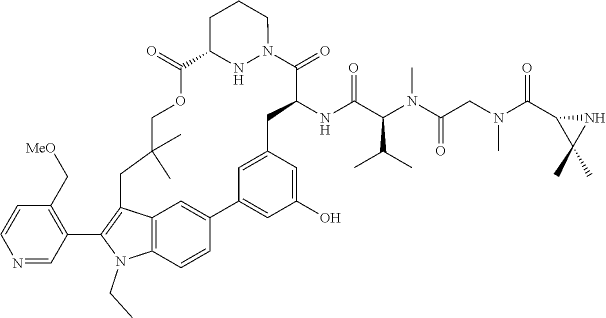 |
| CB8 | 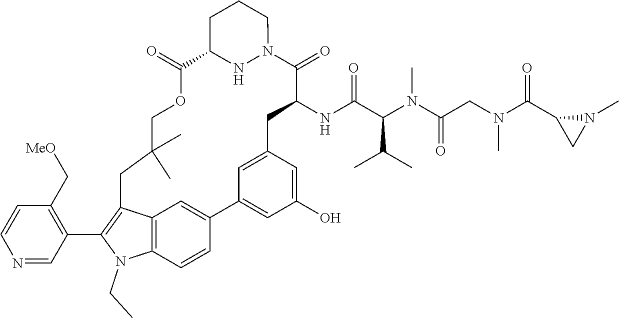 |
| CB9 | 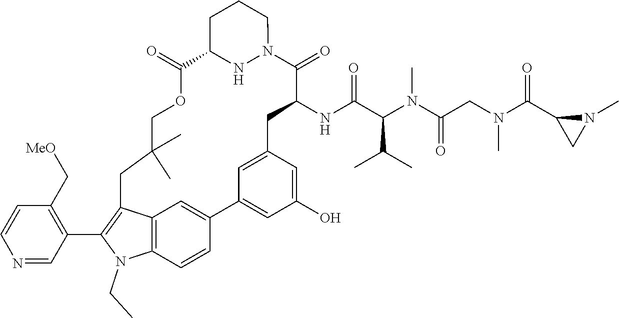 |
| CB16 | 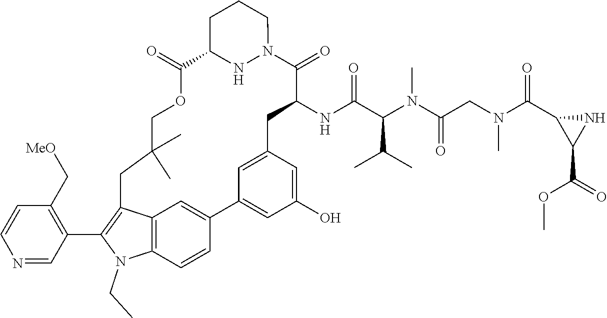 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C(=O)OC)C(=O)N1CCC[C@H](N1)C(=O)OCC(C)(C)C2] |
| CB19 | 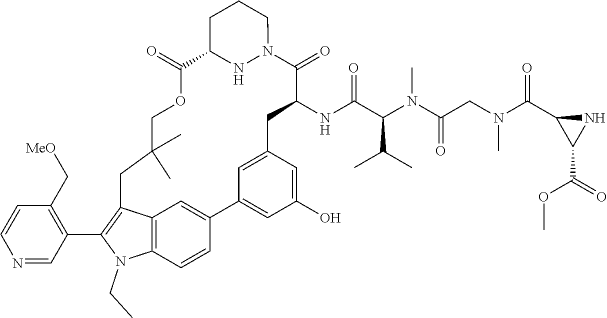 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C(=O)OC)C(=O)N1CCC[C@H](N1)C(=O)OCC(C)(C)C2] |
| CB29 | 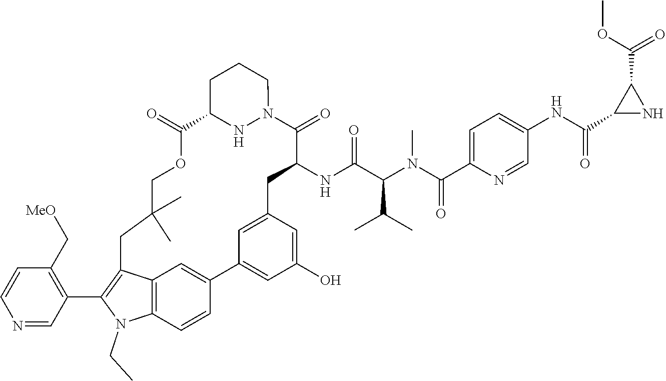 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@H]3C(=O)OC)cn1)C(=O)N1CCC[C@H](N1)C(=O)OCC(C)(C)C2] |
| CB30 | 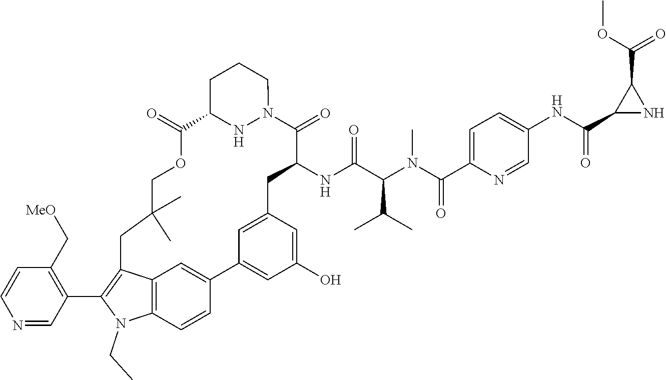 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@@H]3C(=O)OC)cn1)C(=O)N1CCC[C@H](N1)C(=O)OCC(C)(C)C2] |
| CB31 | 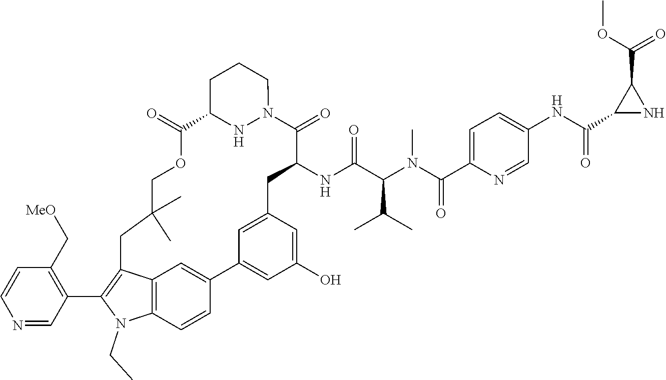 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@@H]3C(=O)OC)cn1)C(=O)N1CCC[C@H](N1)C(=O)OCC(C)(C)C2] |
| CB32 | 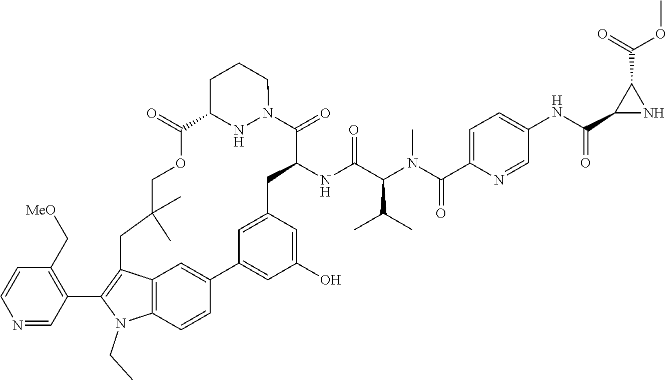 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@H]3C(=O)OC)cn1)C(=O)N1CCC[C@H](N1)C(=O)OCC(C)(C)C2] |
| CB35 | 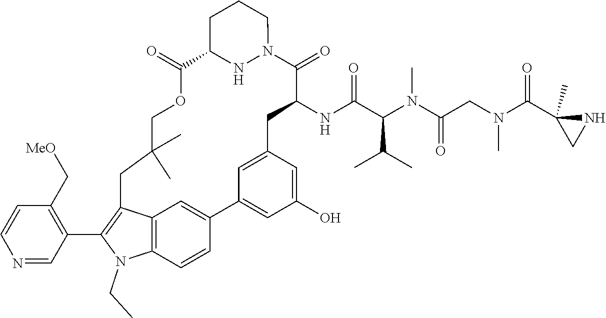 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@]1(C)CN1)C(=O)N1CCC[C@H](N1)C(=O)OCC(C)(C)C2] |
| CB36 | 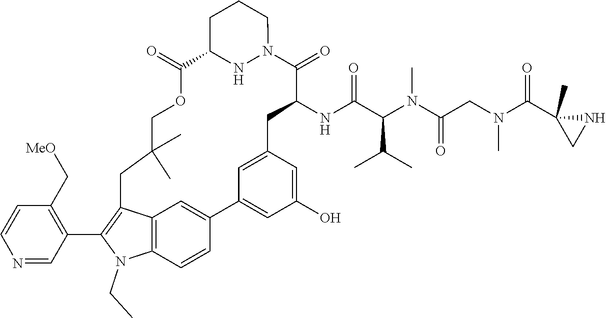 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@]1(C)CN1)C(=O)N1CCC[C@H](N1)C(=O)OCC(C)(C)C2] |
| CB37 | 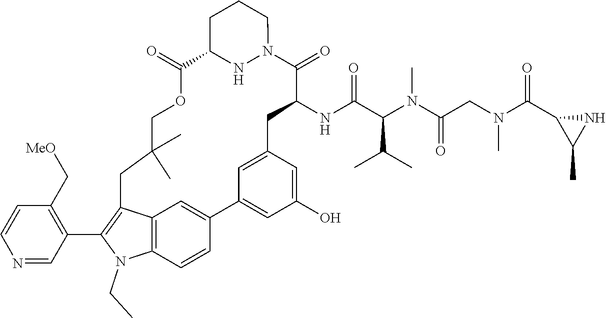 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C)C(=O)N1CCC[C@H](N1)C(=O)OCC(C)(C)C2] |
| CB38 | 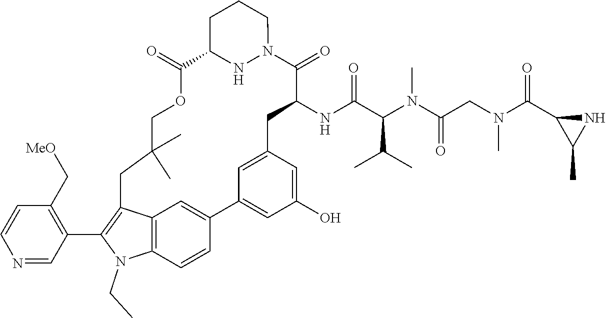 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C)C(=O)N1CCC[C@H](N1)C(=O)OCC(C)(C)C2] |
| CB40 | 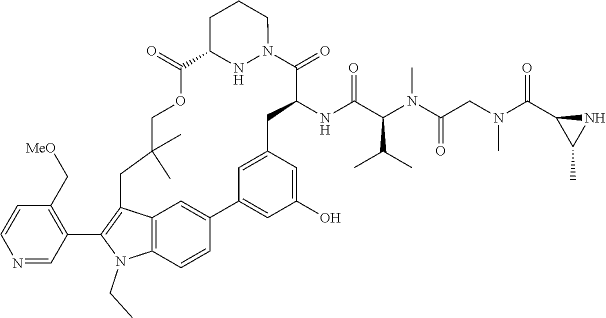 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C)C(=O)N1CCC[C@H](N1)C(=O)OCC(C)(C)C2] |
| CB41 | 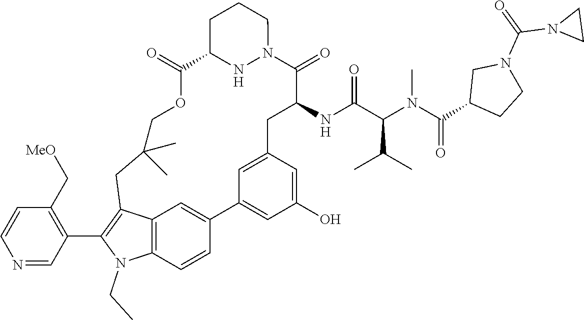 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3CC3)C1)C(=O)N1CCC[C@H](N1)C(=O)OCC(C)(C)C2] |
| CB42 | 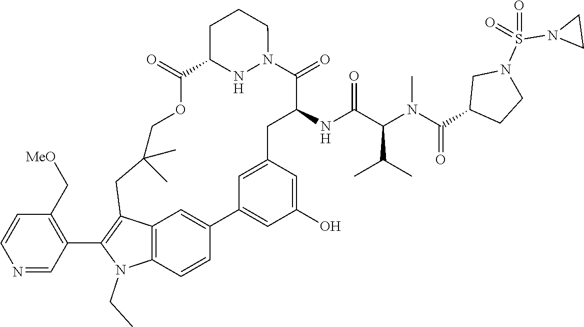 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3CC3)C1)C(=O)N1CCC[C@H](N1)C(=O)OCC(C)(C)C2] |
| CB43 | 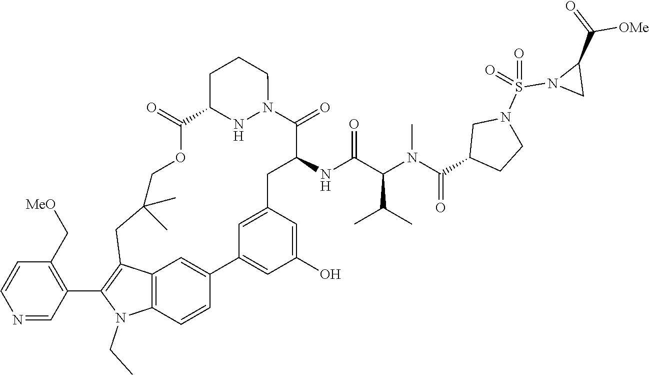 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3C[C@@H]3C(=O)OC)C1)C(=O)N1CCC[C@H](N1)C(=O)OCC(C)(C)C2] |
| CB44 | 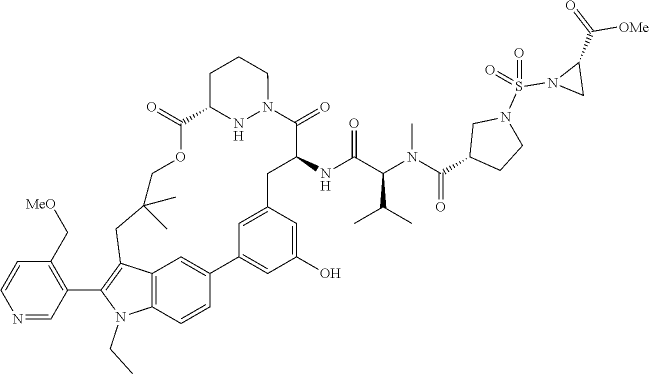 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3C[C@H]3C(=O)OC)C1)C(=O)N1CCC[C@H](N1)C(=O)OCC(C)(C)C2] |
| CB46 | 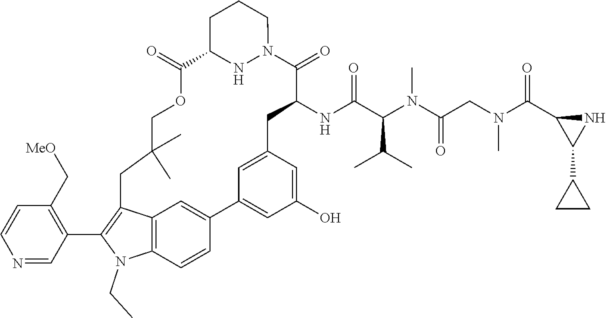 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C1CC1)C(=O)N1CCC[C@H](N1)C(=O)OCC(C)(C)C2] |
| CB48 | 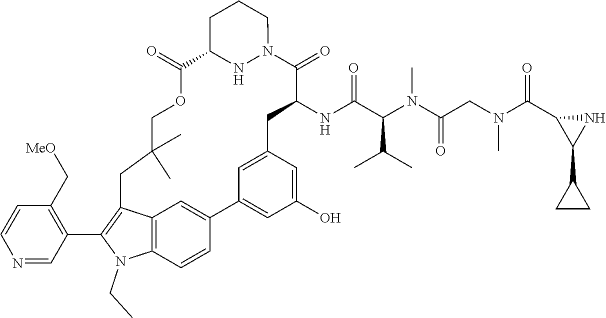 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C1CC1)C(=O)N1CCC[C@H](N1)C(=O)OCC(C)(C)C2] |
| CB51 | 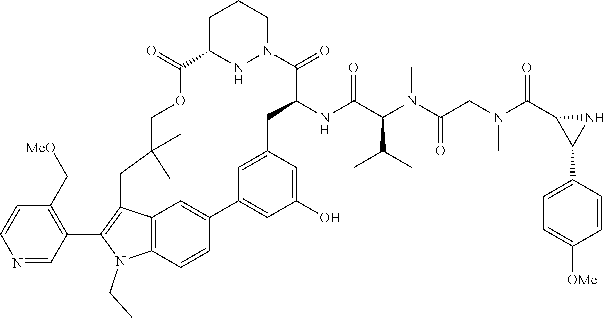 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccc(OC)cc1)C(=O)N1CCC[C@H](N1)C(=O)OCC(C)(C)C2] |
| CB53 | 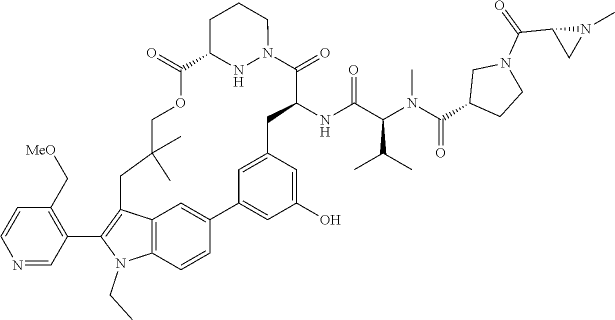 |
| CB54 | 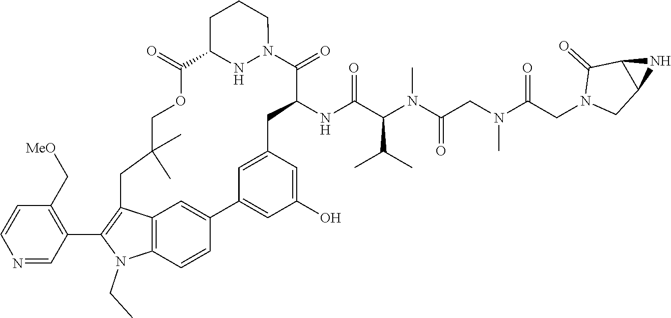 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@@H]3N[C@@H]3C1=O)C(=O)N1CCC[C@H](N1)C(=O)OCC(C)(C)C2] |
| CB55 | 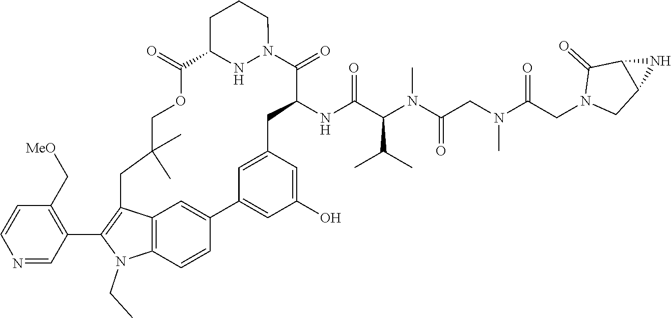 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@H]3N[C@H]3C1=O)C(=O)N1CCC[C@H](N1)C(=O)OCC(C)(C)C2] |
| CB56 | 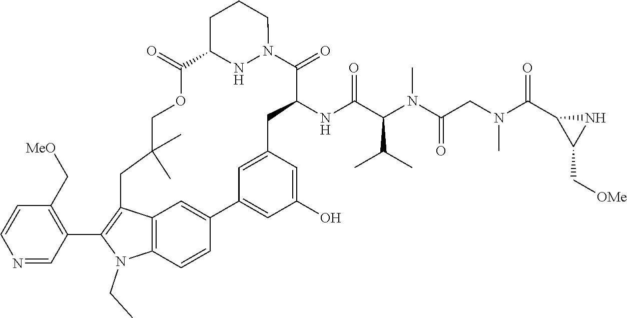 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1COC)C(=O)N1CCC[C@H](N1)C(=O)OCC(C)(C)C2] |
| CB57 | 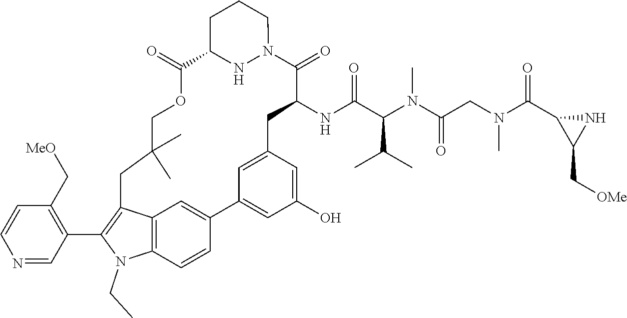 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1COC)C(=O)N1CCC[C@H](N1)C(=O)OCC(C)(C)C2] |
| CB58 | 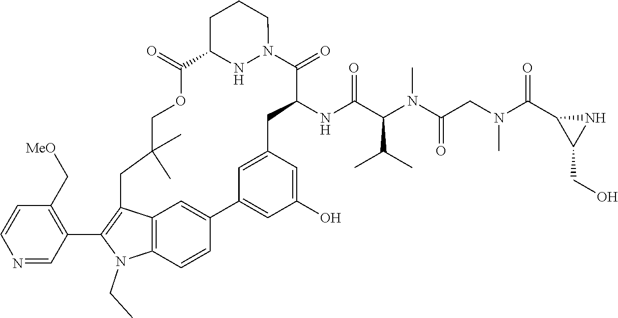 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1CO)C(=O)N1CCC[C@H](N1)C(=O)OCC(C)(C)C2] |
| CB59 | 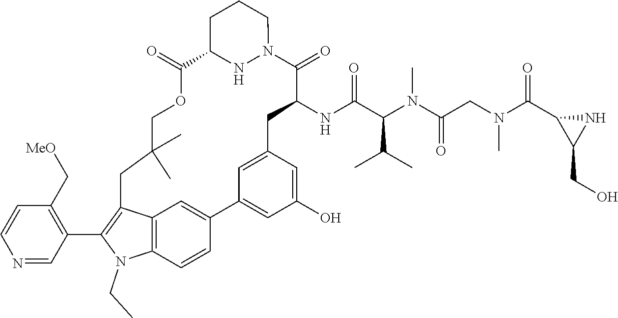 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1CO)C(=O)N1CCC[C@H](N1)C(=O)OCC(C)(C)C2] |
| CB60 | 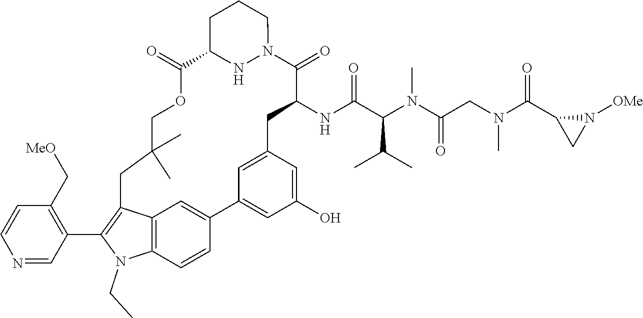 |
| CB61 | 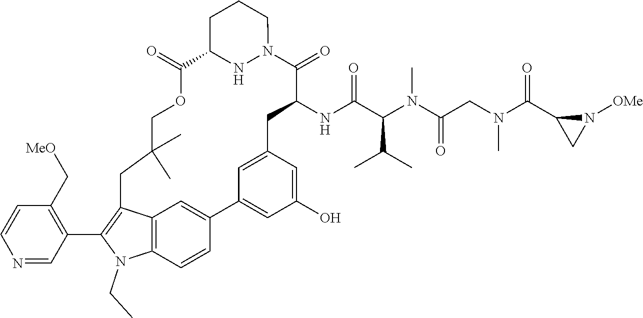 |
| CB62 | 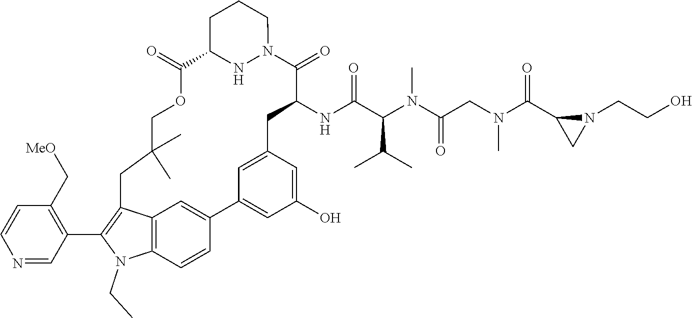 |
| CB63 | 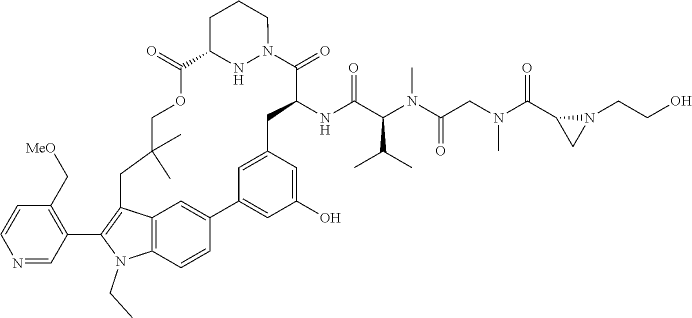 |
| CB73 | 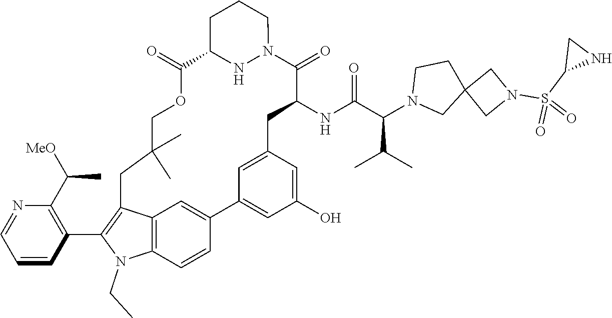 |
| CB74 | 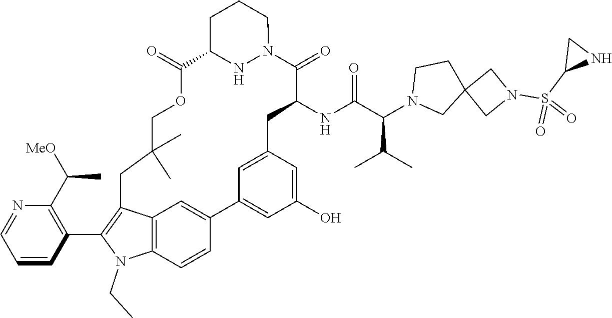 |
| CB76 | 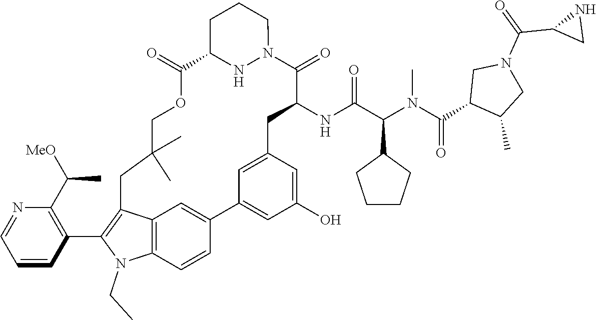 |
| CB78 | 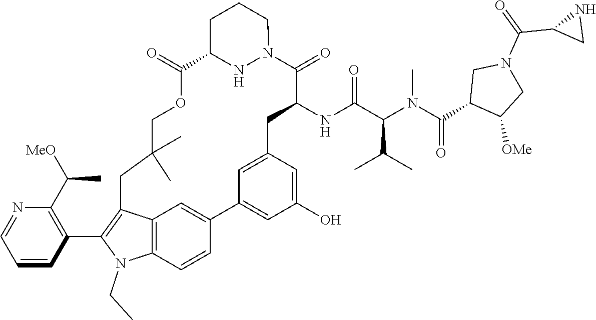 |
| CB79 | 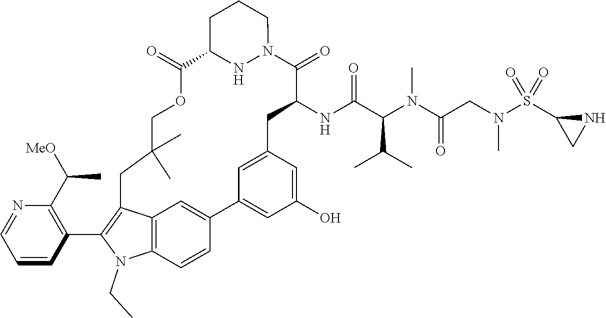 |
| CB80 | 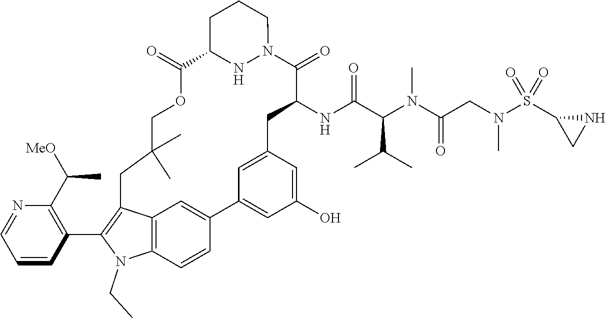 |
| CB83 | 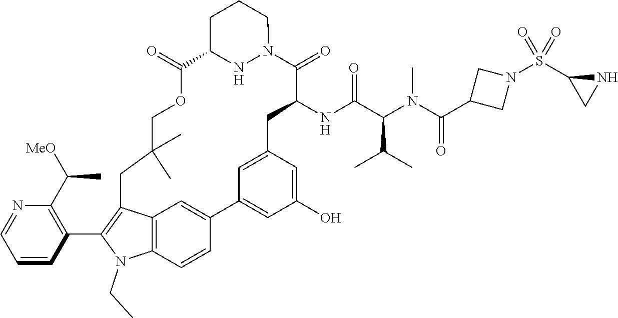 |
| CB84 | 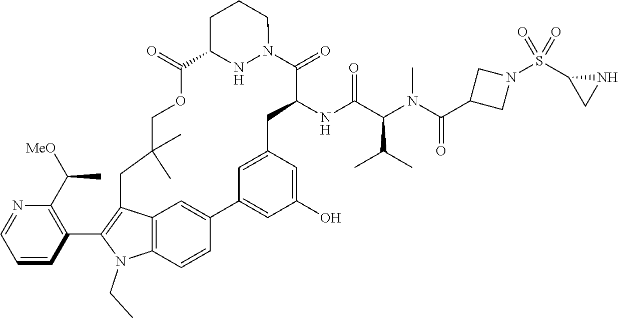 |
| CB87 | 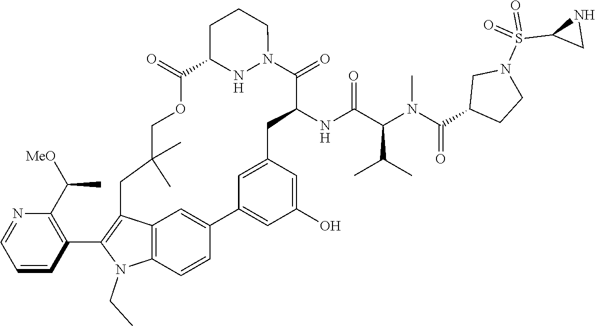 |
| CB88 | 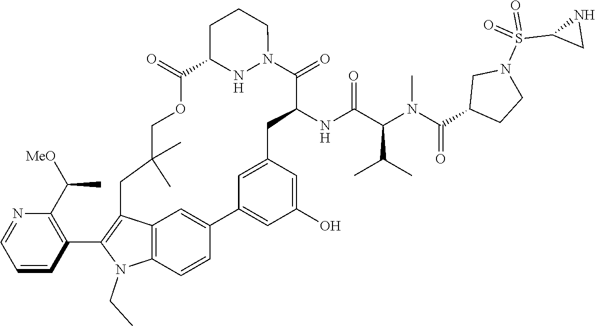 |
| CB91 | 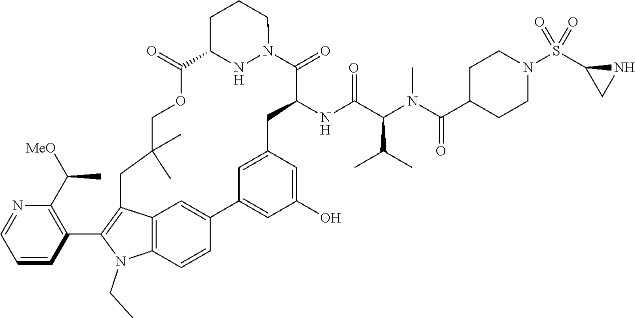 |
| CB92 | 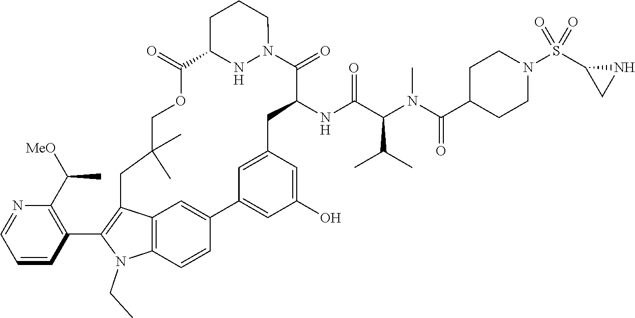 |
| CB97 | 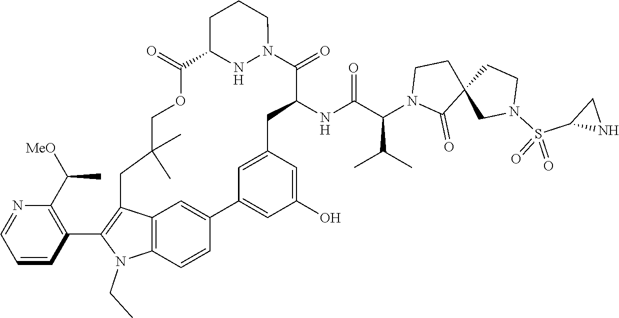 |
| CB98 | 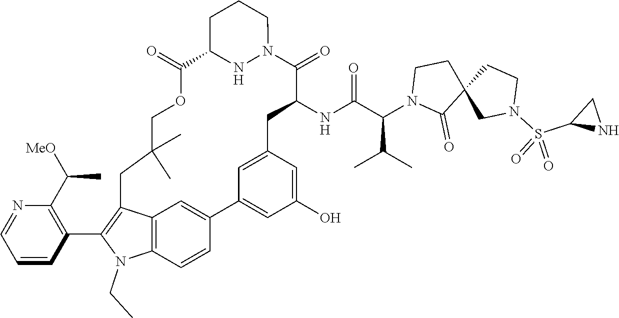 |
| CB101 | 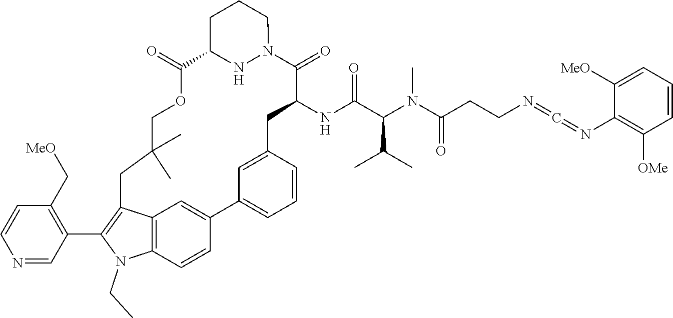 [SMILES: CCn1c(-c2cnccc2COC)c2c3cc(ccc31)-c1cccc(c1)C[C@H](NC(=O)[C@H](C(C)C)N(C)C(=O)CCN=C=Nc1c(OC)cccc1OC)C(=O)N1CCC[C@H](N1)C(=O)OCC(C)(C)C2] |
| CB108 | 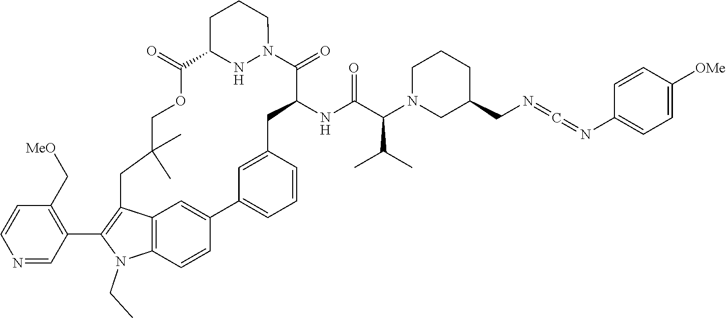 [SMILES: CCn1c(-c2cnccc2COC)c2c3cc(ccc31)-c1cccc(c1)C[C@H](NC(=O)[C@H](C(C)C)N1CCC[C@@H](CN=C=Nc3ccc(OC)cc3)C1)C(=O)N1CCC[C@H](N1)C(=O)OCC(C)(C)C2] |
| CB109 | 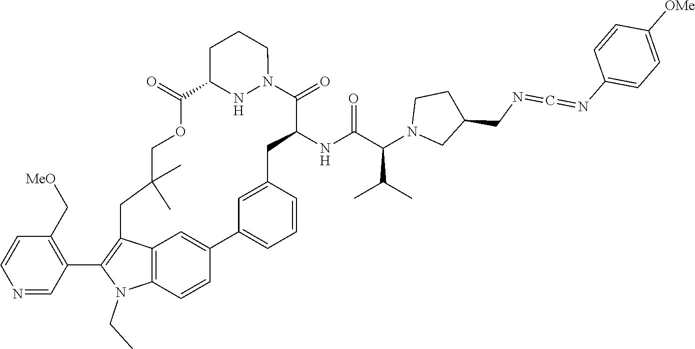 [SMILES: CCn1c(-c2cnccc2COC)c2c3cc(ccc31)-c1cccc(c1)C[C@H](NC(=O)[C@H](C(C)C)N1CC[C@@H](CN=C=Nc3ccc(OC)cc3)C1)C(=O)N1CCC[C@H](N1)C(=O)OCC(C)(C)C2] |
| CB113 | 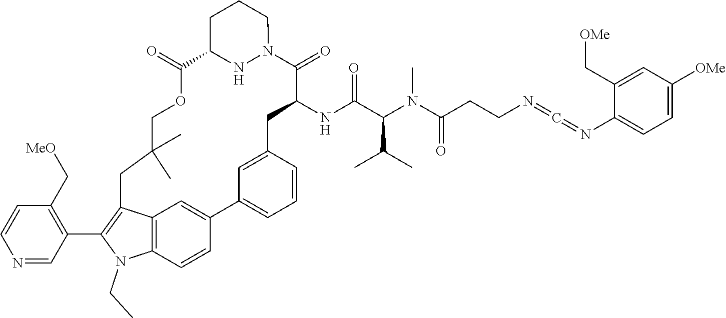 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1COC)C(=O)N1CCC[C@H](N1)C(=O)OCC(C)(C)C2] |
| CB116 | 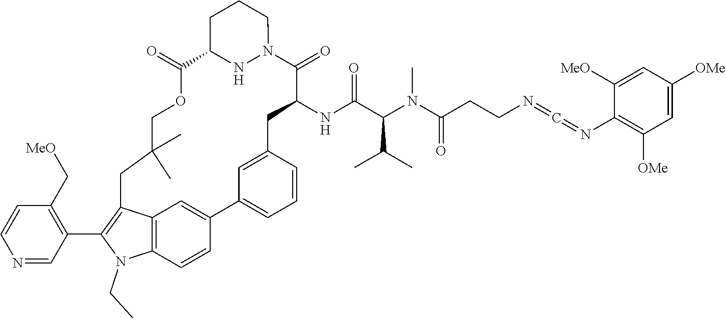 [SMILES: CCn1c(-c2cnccc2COC)c2c3cc(ccc31)-c1cccc(c1)C[C@H](NC(=O)[C@H](C(C)C)N(C)C(=O)CCN=C=Nc1c(OC)cc(OC)cc1OC)C(=O)N1CCC[C@H](N1)C(=O)OCC(C)(C)C2] |
| CB117 | 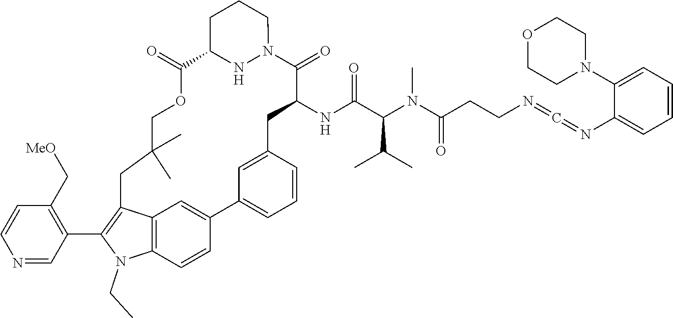 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1N1CCOCC1)C(=O)N1CCC[C@H](N1)C(=O)OCC(C)(C)C2] |
| CB118 | 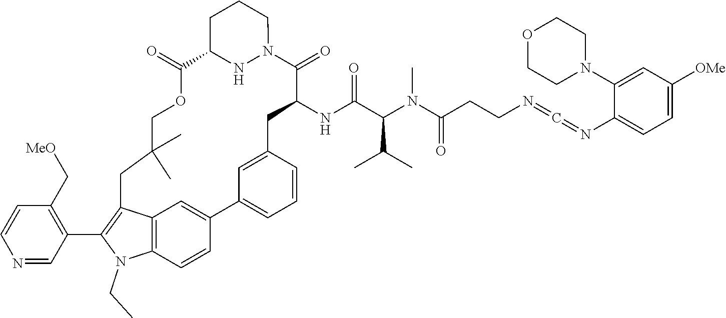 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1N1CCOCC1)C(=O)N1CCC[C@H](N1)C(=O)OCC(C)(C)C2] |
| CB119 | 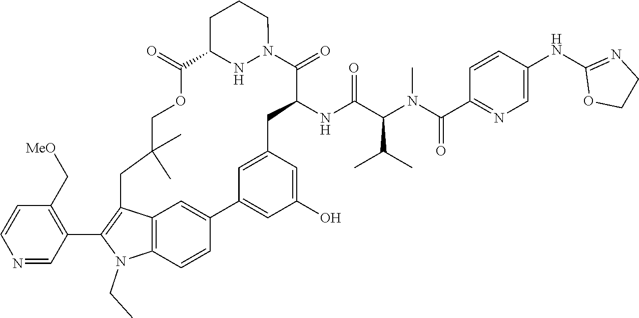 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC3=NCCO3)cn1)C(=O)N1CCC[C@H](N1)C(=O)OCC(C)(C)C2] |
| CB120 | 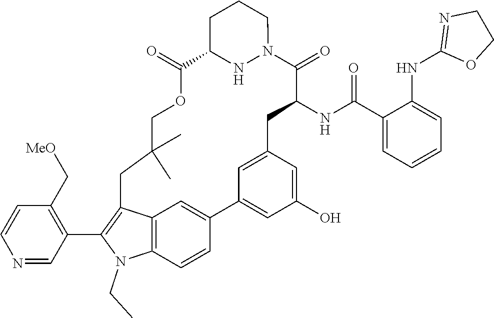 [SMILES: CCn1c(-c2cnccc2COC)c2c3cc(ccc31)-c1cc(O)cc(c1)C[C@H](NC(=O)c1ccccc1NC1=NCCO1)C(=O)N1CCC[C@H](N1)C(=O)OCC(C)(C)C2] |
| CB122 | 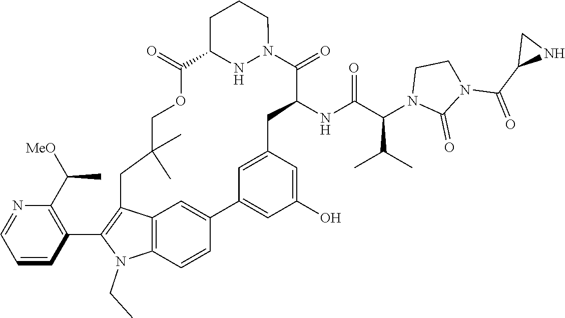 |
| CB123 | 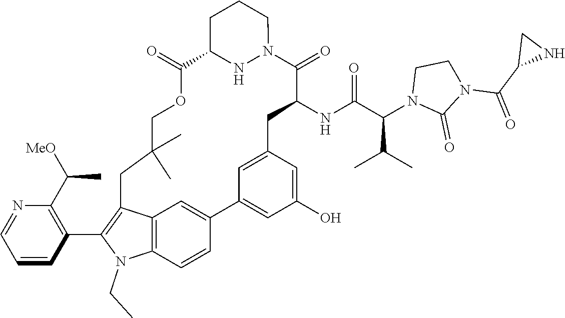 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, the RAS(ON) inhibitor is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

In some embodiments, the RAS(ON) inhibitor is provided as a conjugate, or salt thereof, comprising the structure of Formula CIV:

M-L-P Formula CIV
wherein L is a linker;
P is a monovalent organic moiety; and
M has the structure of Formula CVa:

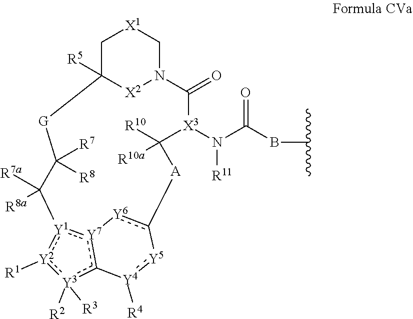

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the conjugate has the structure of Formula CIV:

M-L-P Formula CIV
wherein L is a linker;
P is a monovalent organic moiety; and
M has the structure of Formula CVb:

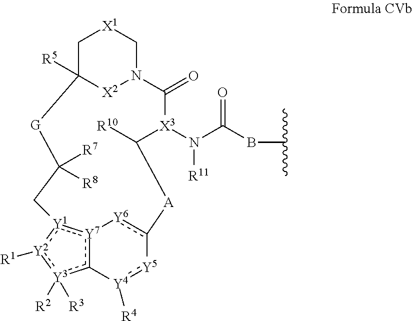

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, the conjugate has the structure of Formula CIV:

M-L-P Formula CIV
wherein L is a linker;
P is a monovalent organic moiety; and
M has the structure of Formula CVc:

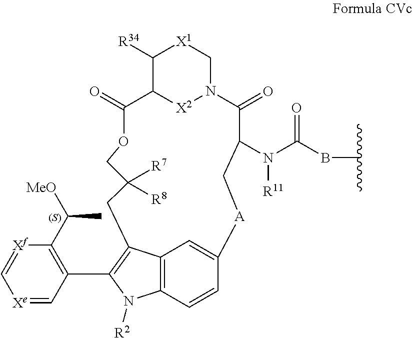

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
X^{e} and X^{f} are, independently, N or CH;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹¹ is hydrogen or C₁-C₃ alkyl; and
R³⁴ is hydrogen or C₁-C₃ alkyl.

In some embodiments of Formula CI and subformula thereof, X^{e} is N and X^{f} is CH. In some embodiments, X^{e} is CH and X^{f} is N.

In some embodiments, the conjugate has the structure of Formula CIV:

M-L-P Formula CIV
wherein L is a linker;
P is a monovalent organic moiety; and
M has the structure of Formula CVd:

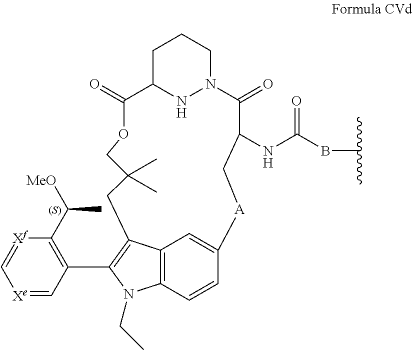

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
X^{e} and X^{f} are, independently, N or CH.

In some embodiments of Formula C₁ and subformula thereof, X^{e} is N and X^{f} is CH. In some embodiments, X^{e} is CH and X^{f} is N.

In some embodiments of conjugates of formula CIV, the linker has the structure of Formula CII:

A¹-(B¹)_{f}—(C¹)_{g}—(B²)ₕ-(D¹)-(B³)ᵢ—(C²)ⱼ—(B⁴)ₖ-A² Formula CII

where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments of conjugates of the present invention, the linker is bound to the monovalent organic moiety through a bond to a carboxyl group of an amino acid residue of the monovalent organic moiety.

In some embodiments of conjugates of formula CIV, the monovalent organic moiety is a protein. In some embodiments, the protein is a Ras protein. In some embodiments, the Ras protein is K-Ras G12D or K-Ras G13D.

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below and in WO 2021/091967.

Compounds of Table C1 herein were prepared using methods disclosed herein or were prepared using methods disclosed herein combined with the knowledge of one of skill in the art. Compounds of Table C2 may be prepared using methods disclosed herein or may be prepared using methods disclosed herein combined with the knowledge of one of skill in the art.

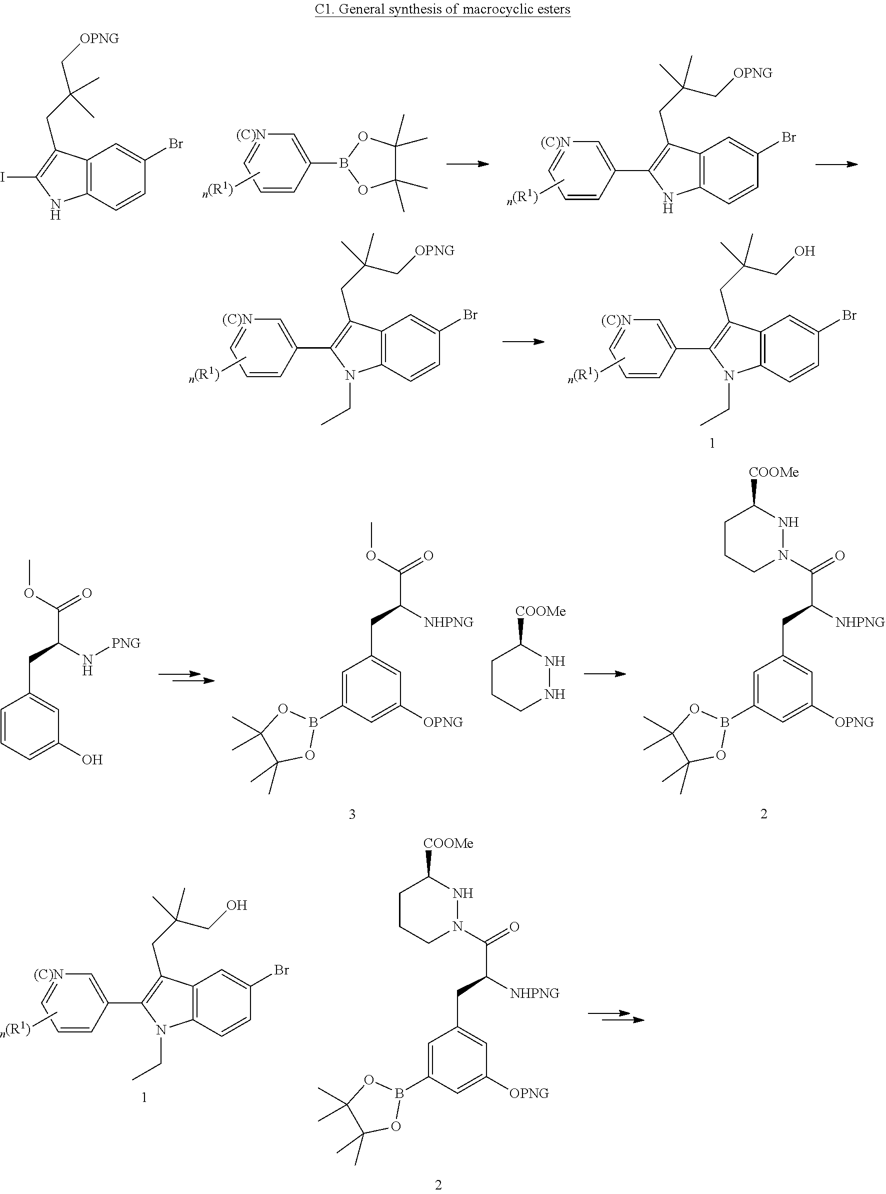

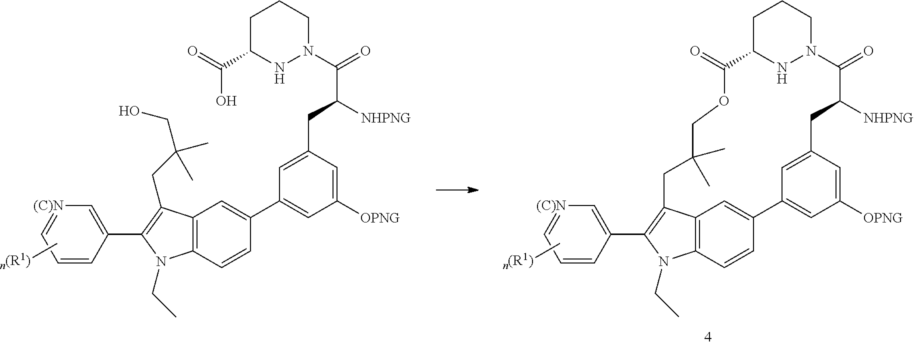

A general synthesis of macrocyclic esters is outlined in Scheme C1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions.

Methyl-amino-hexahydropyridazine-3-carboxylate-boronic ester (2) can be prepared in three steps, including protection, iridium catalyst mediated borylation, and coupling with methyl (S)-hexahydropyridazine-3-carboxylate.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of Pd catalyst followed by hydrolysis and macrolactonization steps to result in an appropriately protected macrocyclic intermediate (4). Additional deprotection or functionalization steps are required to produce a final compound. For example, a person of skill in the art would be able to install into a macrocyclic ester a desired —B-L-W group of a compound of Formula (CI), where B, L and W are defined herein, including by using methods exemplified in certain Schemes below and in the Example section herein.

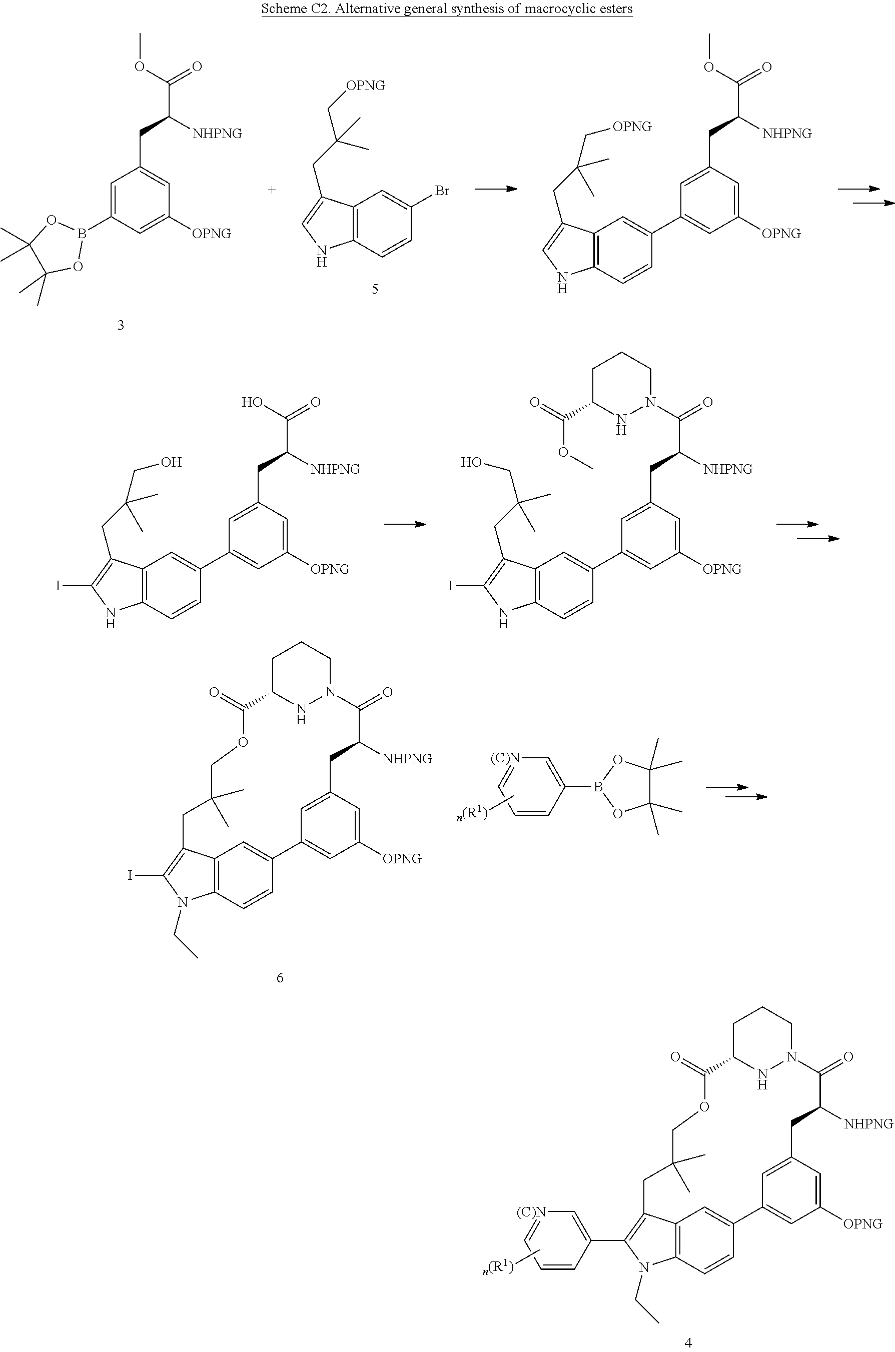

Alternatively, macrocyclic esters can be prepared as described in Scheme C2. An appropriately protected bromo-indolyl (5) can be coupled in the presence of Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (6). Coupling in the presence of Pd catalyst with an appropriately substituted boronic ester can yield fully a protected macrocycle (4). Additional deprotection or functionalization steps are required to produce a final compound. For example, a person of skill in the art would be able to install into a macrocyclic ester a desired —B-L-W group of a compound of Formula (CI), where B, L and W are defined herein, including by using methods exemplified in certain Schemes below and in the Example section herein.

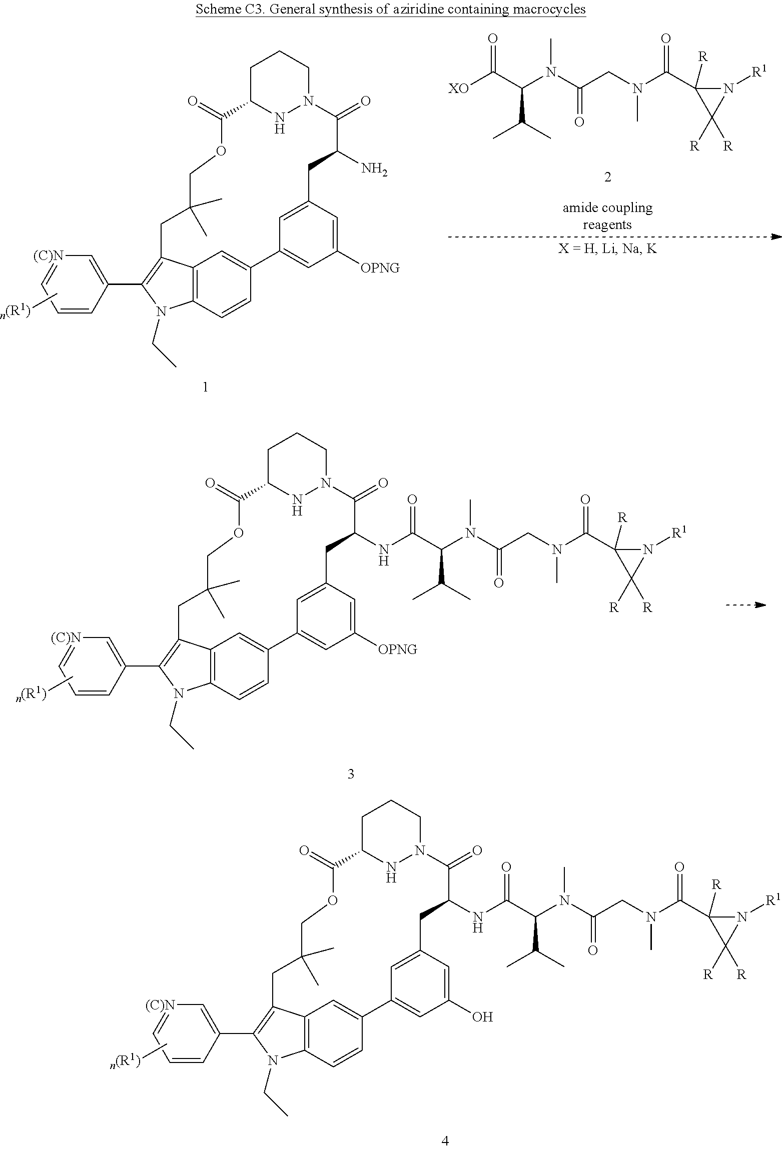

As shown in Scheme C3, compounds of this type may be prepared by the reaction of an appropriate amine (1) with an aziridine containing carboxylic acid (2) in the presence of standard amide coupling reagents, followed by deprotection of the aziridine, if R¹ is a protecting group, and deprotection of the phenol, if required, to produce the final compound (4).

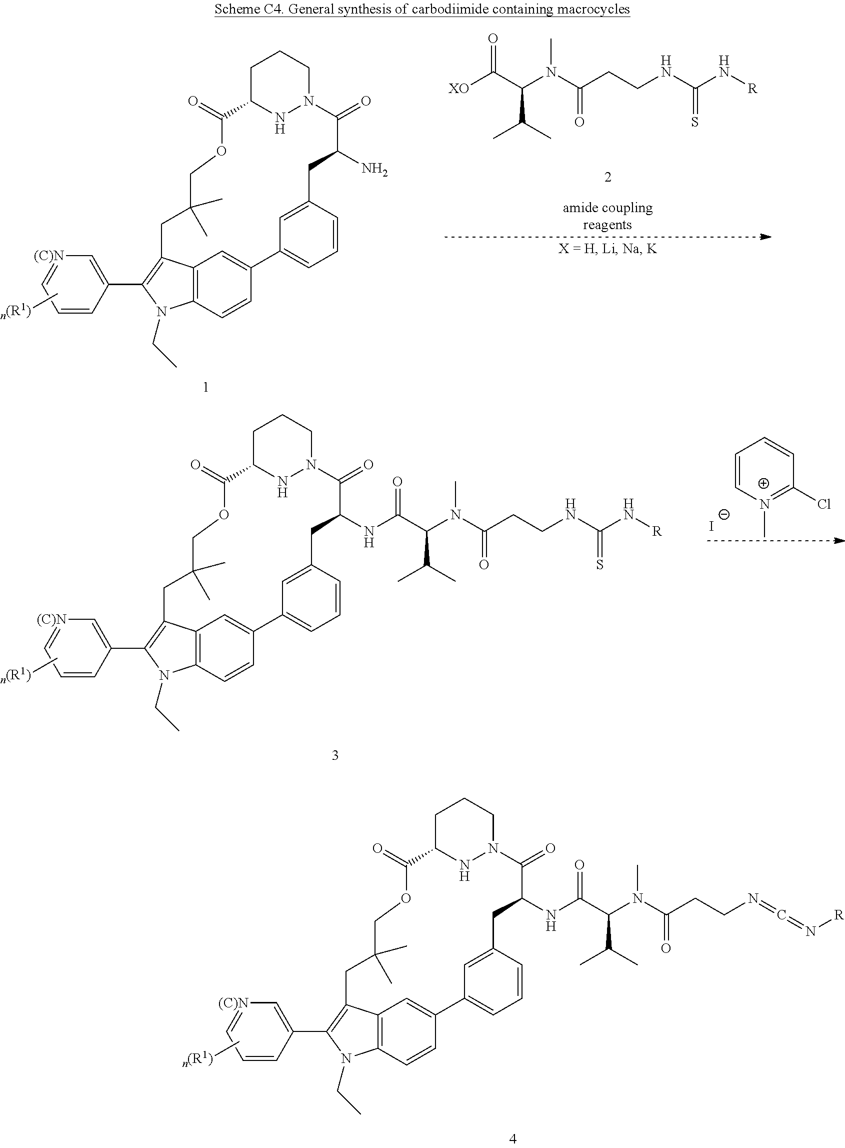

As shown in Scheme C4, compounds of this type may be prepared by the reaction of an appropriate amine (1) with a thiourea containing carboxylic acid (2) in the presence of standard amide coupling reagents, followed by conversion of the thiourea (3) to a carbodiimide (4) in the presence of 2-chloro-1-methylpyridin-1-ium iodide.

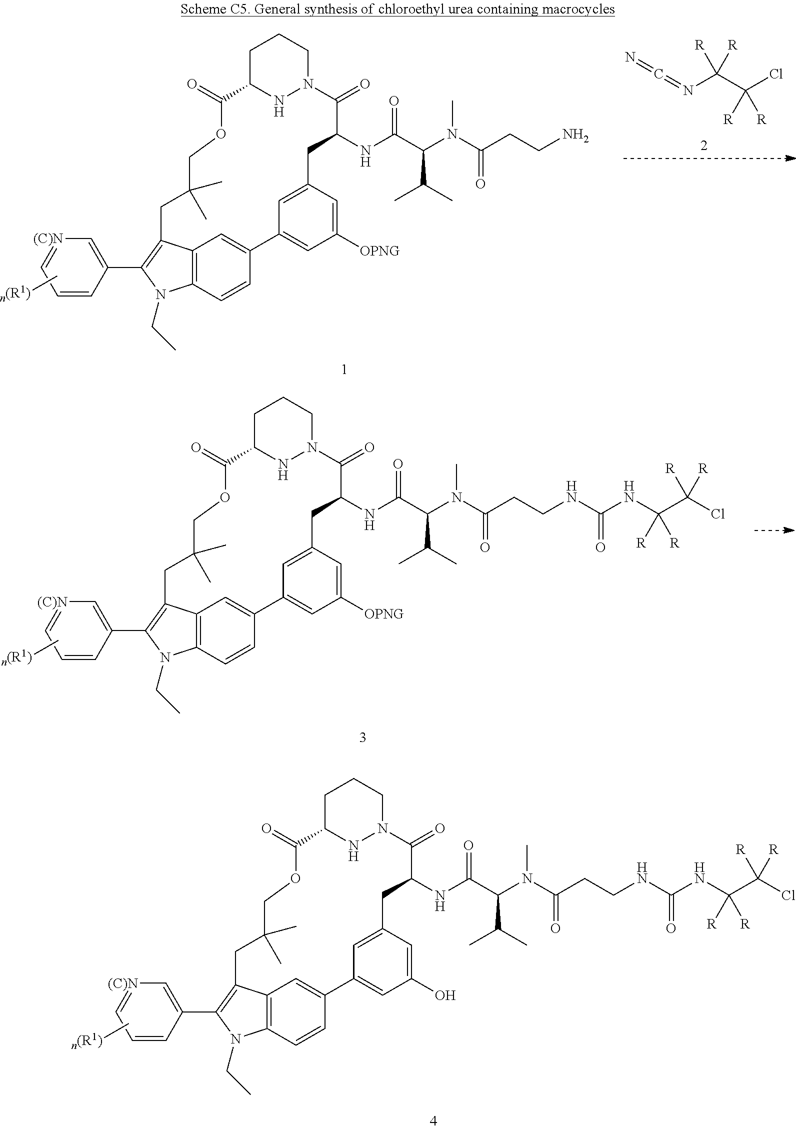

As shown in Scheme C5, compounds of this type may be prepared by the reaction of an appropriate amine (1) with an isocyanate (2) under basic conditions, followed by deprotection of the phenol, if required, to produce the final compound (4).

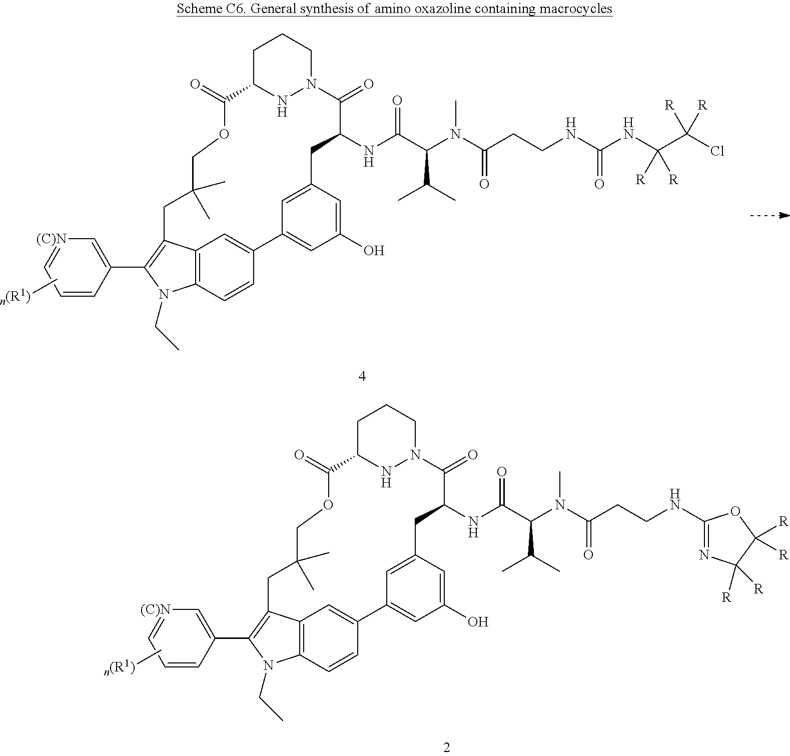

P As shown in Scheme C6, compounds of this type may be prepared by cyclization of an appropriate chloroethyl urea (1) under elevated temperatures to produce the final compound (2).

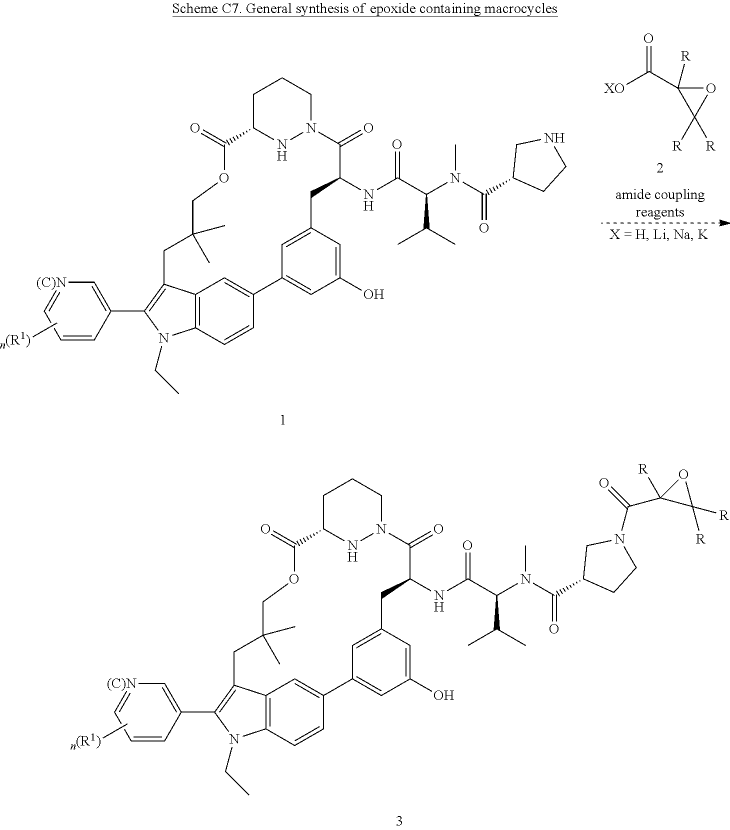

As shown in Scheme C7, compounds of this type may be prepared by the reaction of an appropriate amine (1) with an epoxide containing carboxylic acid (2) in the presence of standard amide coupling reagents to produce the final compound (3).

In addition, compounds of the disclosure can be synthesized using the methods described in the WO 2021/091967, together with synthetic methods known in the art of synthetic organic chemistry, or variations thereon as appreciated by those skilled in the art. These methods include but are not limited to those methods described in the WO 2021/091967. For example, a person of skill in the art would be able to install into a macrocyclic ester a desired —B-L-W group of a compound of Formula (CI), where B, L and W are defined herein, including by using methods exemplified in certain Schemes above and in the Example section herein.

In some embodiments, the RAS(ON) inhibitor is a compound, or a pharmaceutically acceptable salt thereof, having the structure of Formula DIa:

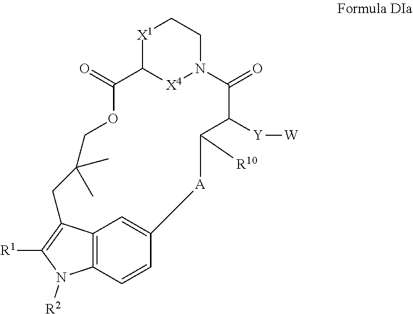

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;

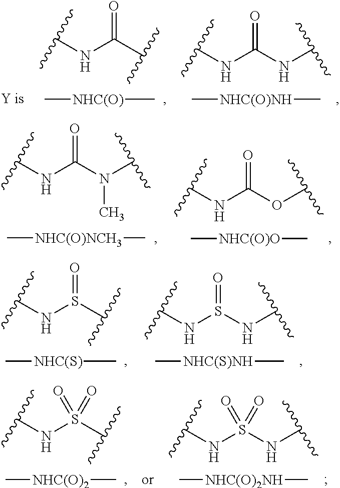

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

In some embodiments, the RAS(ON) inhibitor, or pharmaceutically acceptable salt thereof, has the structure of Formula DIa-2:

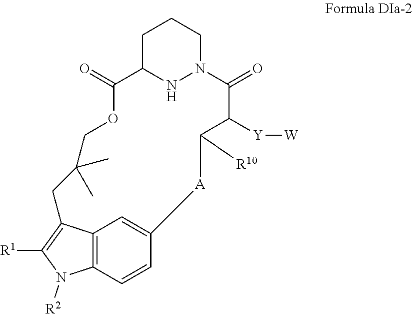

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
Y is

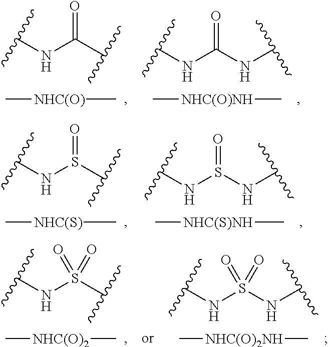

W is hydrogen, 01-C₄ alkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R¹⁰ is hydrogen or optionally substituted C₁-C₆ heteroalkyl. In some embodiments, R¹⁰ is hydrogen.

In some embodiments of Formula DIa and subformula thereof, R¹ is optionally substituted 6 to 10-membered aryl or optionally substituted 5 to 10-membered heteroaryl. In some embodiments, R¹ is optionally substituted phenyl or optionally substituted pyridine.

In some embodiments of Formula DIa and subformula thereof, A is optionally substituted thiazole, optionally substituted triazole, optionally substituted morpholino, optionally substituted piperidinyl, optionally substituted pyridine, or optionally substituted phenyl. In some embodiments, A is optionally substituted thiazole, optionally substituted triazole, optionally substituted morpholino, or phenyl. In some embodiments, A is not an optionally substituted phenyl or benzimidazole. In some embodiments, A is not hydroxyphenyl.

In some embodiments of Formula DIa and subformula thereof, Y is —NHC(O)— or —NHC(O)NH—.

In some embodiments, the RAS(ON) inhibitor, or a pharmaceutically acceptable salt thereof, has the structure of Formula DIIa:

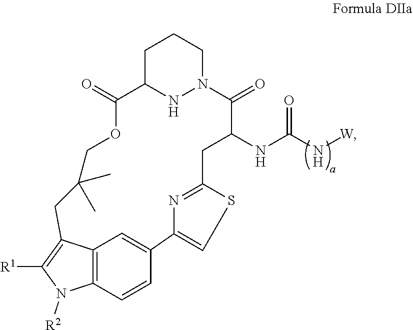

wherein W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
a is 0 or 1.

In some embodiments, the RAS(ON) inhibitor, or a pharmaceutically acceptable salt thereof, has the structure of Formula DIIa-1:

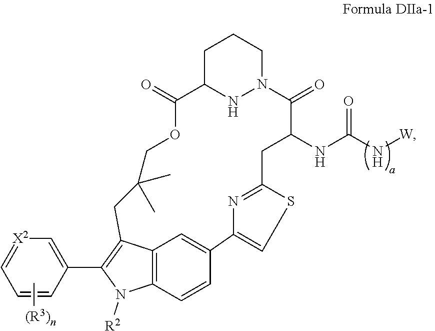

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;

### X² is N or CH;

R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIa-2:

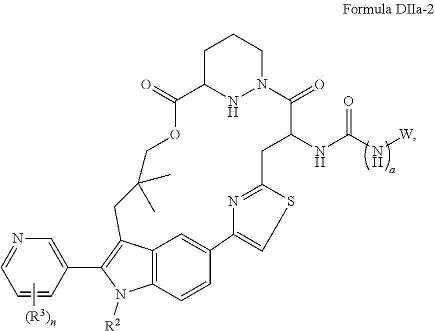

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIa-3:

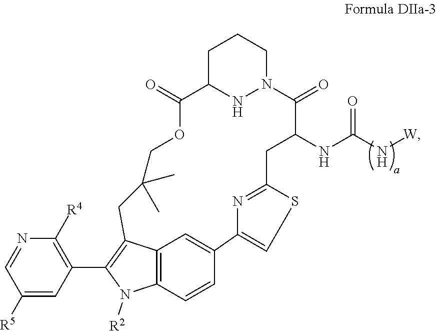

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;

### X² is N or CH;

R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIa-4:

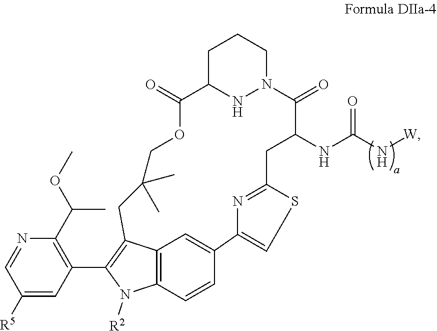

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;

### X² is N or CH;

R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIa-5:

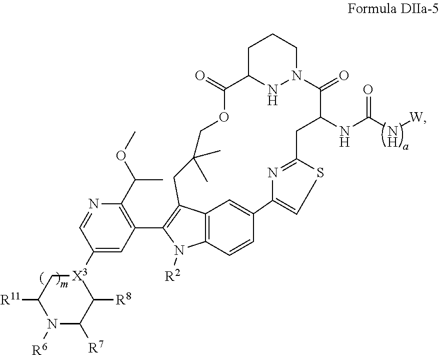

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;

### X² is N or CH;

R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is H. In some embodiments, X³ is N, m is 1, and R¹¹ is H.

In some embodiments, the RAS(ON) inhibitor, or a pharmaceutically acceptable salt thereof, has the structure of Formula DIIa-6:

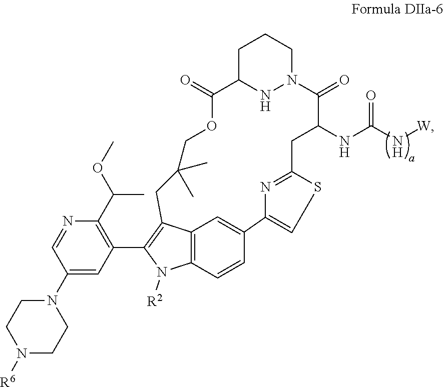

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;

### X² is N or CH;

R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIa-7:

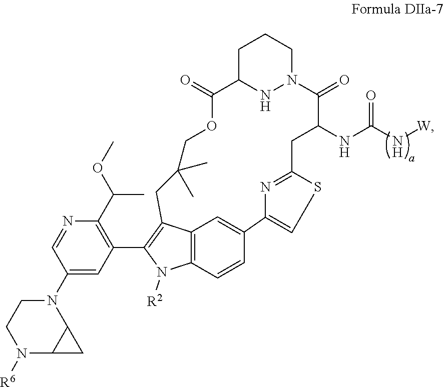

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;

### X² is N or CH;

R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments (e.g., of any one of Formulae DIIa-6 or DIIa-7), R⁶ is methyl.

In some embodiments, the RAS(ON) inhibitor, or a pharmaceutically acceptable salt thereof, has the structure of Formula DIIa-8 or Formula DIIa-9:

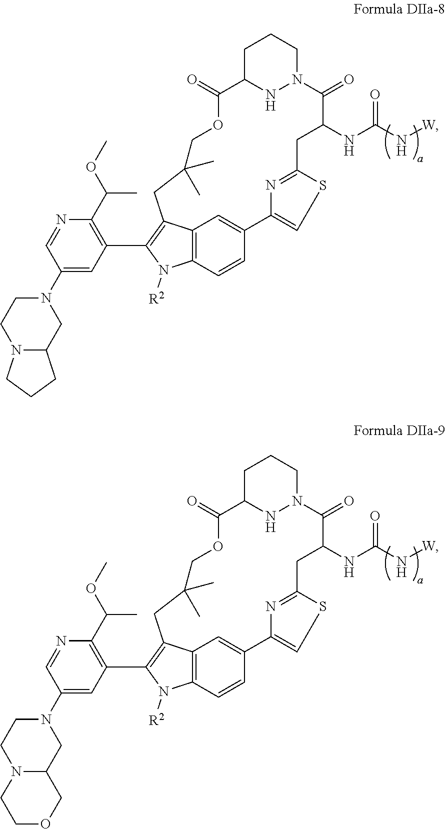

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl.

In some embodiments, the RAS(ON) inhibitor, or a pharmaceutically acceptable salt thereof, has the structure of Formula DIIIa:

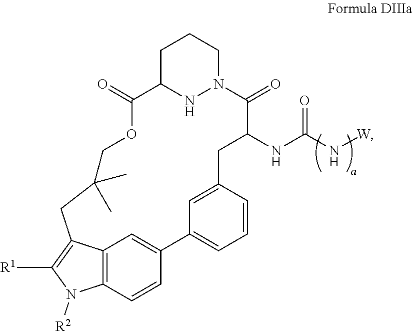

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl.

In some embodiments, the RAS(ON) inhibitor, or a pharmaceutically acceptable salt thereof, has the structure of Formula DIIIa-1:

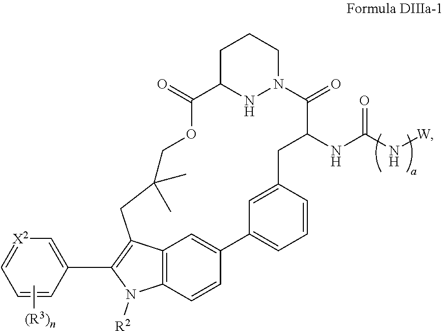

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIIa-2:

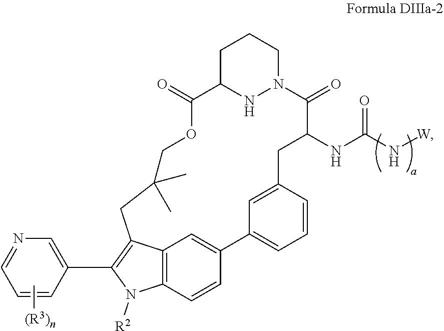

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIIa-3:

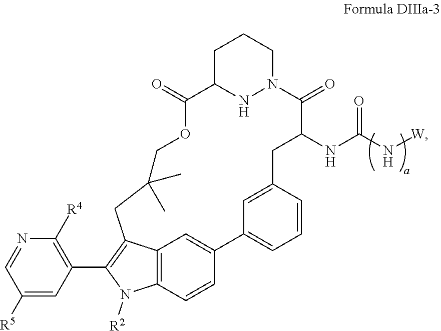

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIIa-4:

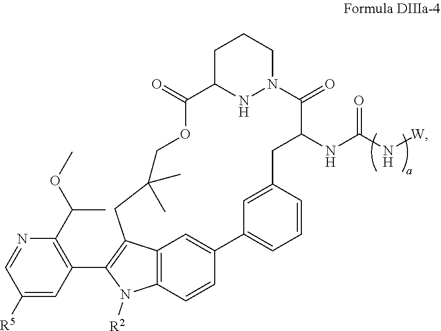

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIIa-5:

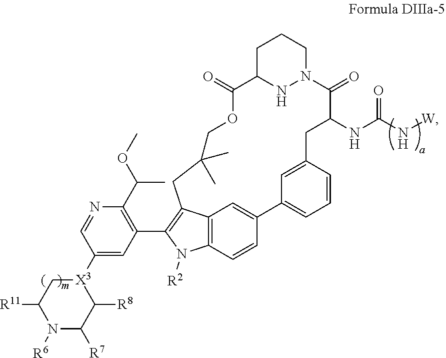

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DIIIa-6:

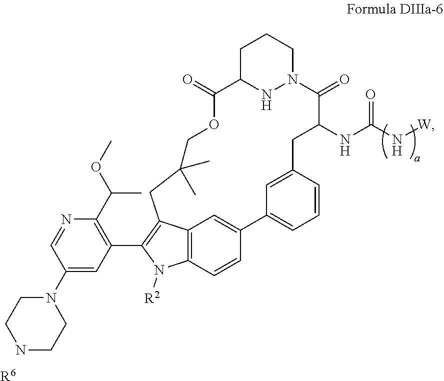

wherein a is 0 or;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIIa-7:

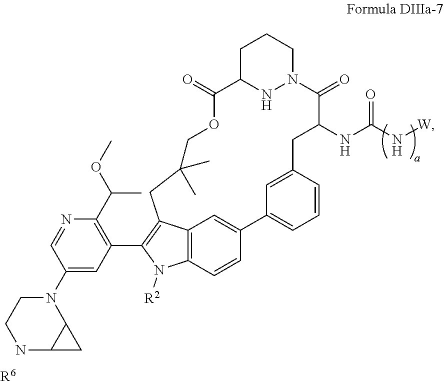

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments (e.g., of any one of Formulae DIIIa-6 or DIIIa-7), R⁶ is methyl.

In some embodiments, the RAS(ON) inhibitor, or a pharmaceutically acceptable salt thereof, has the structure of Formula DIIIa-8 or Formula DIIIa-9:

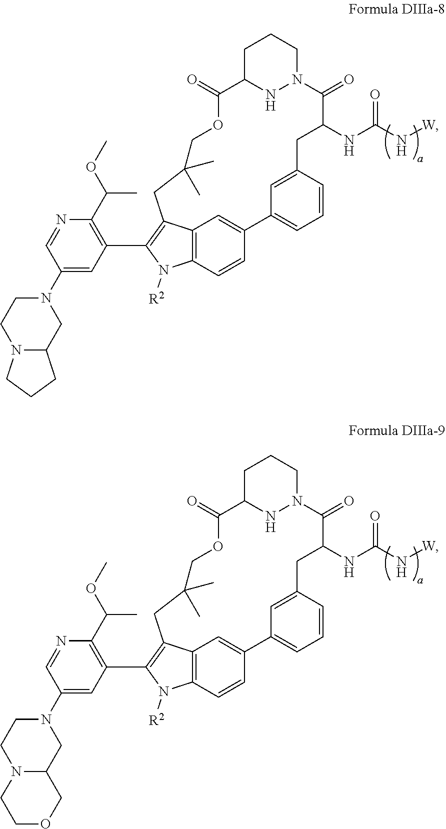

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl.

In some embodiments, the RAS(ON) inhibitor, or a pharmaceutically acceptable salt thereof, has the structure of Formula DIVa:

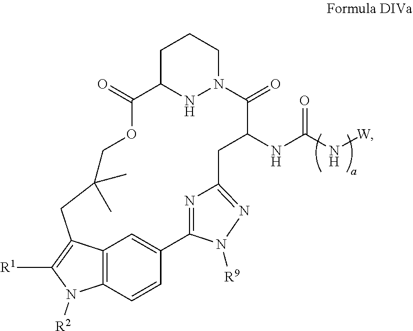

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
a is 0 or 1.

In some embodiments, the RAS(ON) inhibitor, or a pharmaceutically acceptable salt thereof, has the structure of Formula DIVa-1:

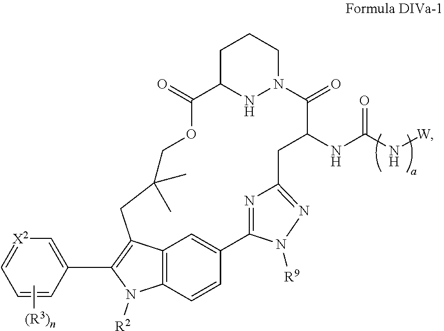

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl;
R⁹ is H or C₁-C₆ alkyl; and
n is an integer from 1 to 4.

In some embodiments, the RAS(ON) inhibitor, or a pharmaceutically acceptable salt thereof, has the structure of Formula DIVa-2:

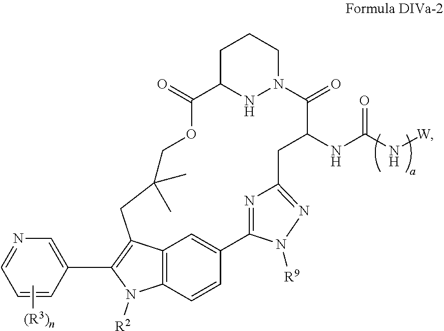

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl;
R⁹ is H or C₁-C₆ alkyl; and
n is an integer from 1 to 4.

In some embodiments, the RAS(ON) inhibitor, or a pharmaceutically acceptable salt thereof, has the structure of Formula DIVa-3:

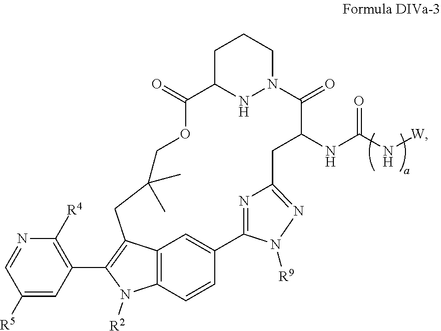

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl; and
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIVa-4:

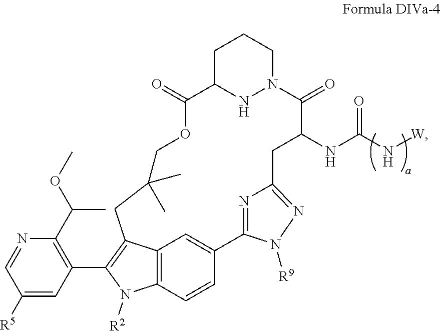

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIVa-5:

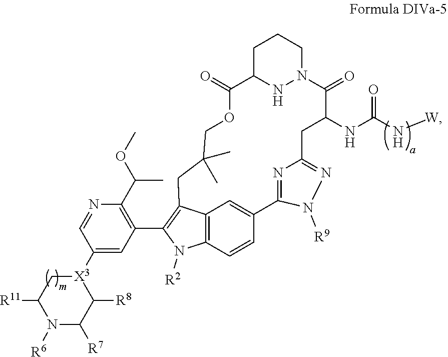

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X³ is N or CH;
m is 1 or 2;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆alkyl;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DIVa-6:

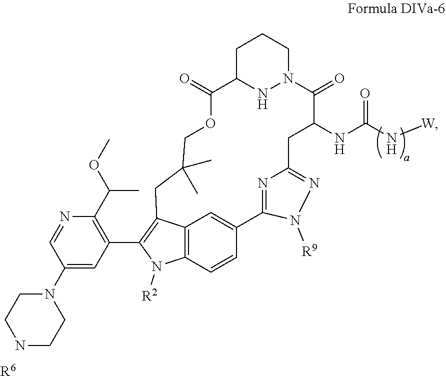

In some embodiments, the RAS(ON) inhibitor, or a pharmaceutically acceptable salt thereof, has the structure of Formula DIVa-7:

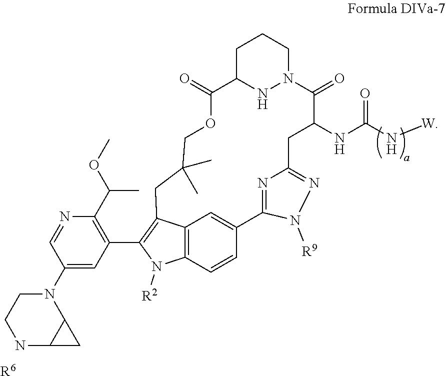

In some embodiments (e.g., of any one of Formulae DIVa-6 or DIVa-7), R⁶ is methyl.

In some embodiments, the RAS(ON) inhibitor, or a pharmaceutically acceptable salt thereof, has the structure of Formula DIVa-8 or Formula DIVa-9:

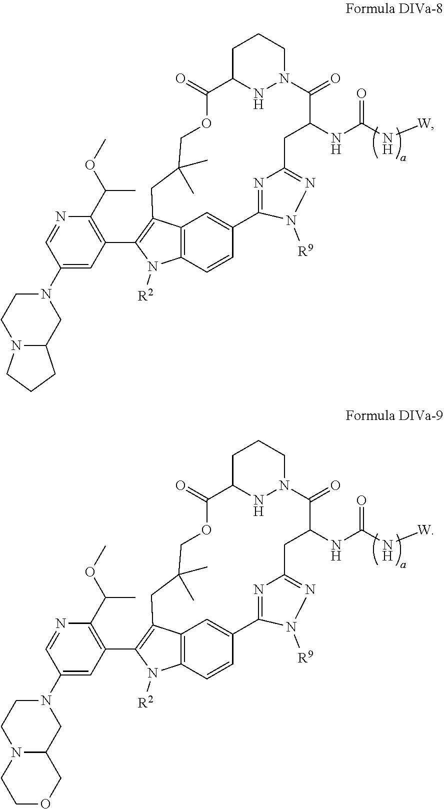

In some embodiments (e.g., of any one of Formulae DIVa, DIVa-1, DIVa-2, DIVa-3, DIVa-4, DIVa-5, DIVa-6, DIVa-7, DIVa-8, or DIVa-9), R⁹ is methyl.

In some embodiments, Y is —NHS(O)₂— or —NHS(O)₂NH—.

In some embodiments, the RAS(ON) inhibitor, or a pharmaceutically acceptable salt thereof, has the structure of Formula DVa:

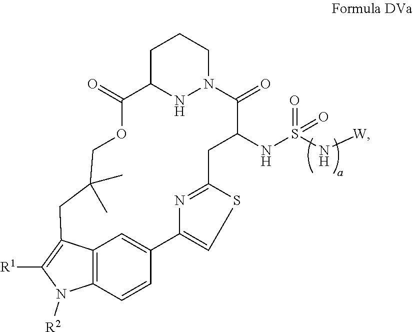

wherein a is 0 or 1.

In some embodiments, the RAS(ON) inhibitor, or a pharmaceutically acceptable salt thereof, has the structure of Formula DVa-1:

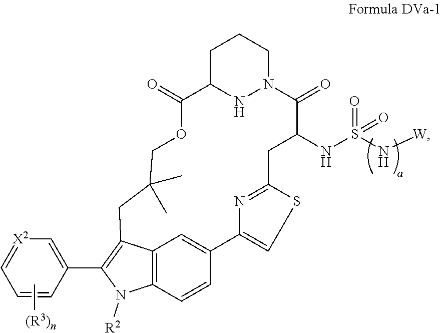

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVa-2:

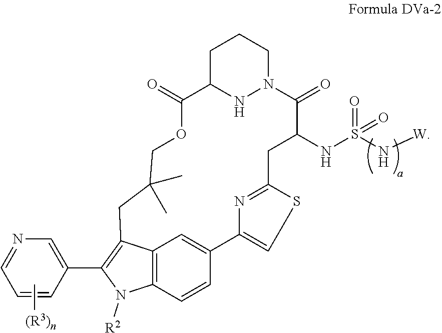

In some embodiments, the RAS(ON) inhibitor, or a pharmaceutically acceptable salt thereof, has the structure of Formula DVa-3:

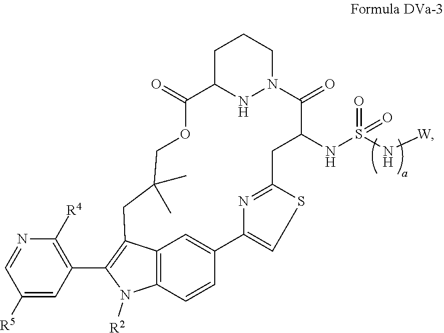

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVa-4:

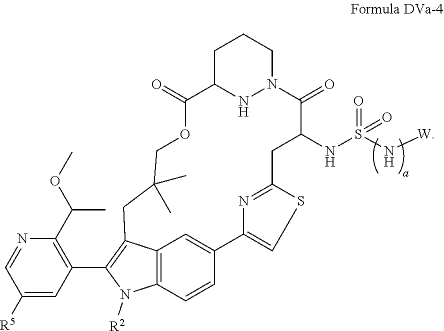

In some embodiments, the RAS(ON) inhibitor, or a pharmaceutically acceptable salt thereof, has the structure of Formula DVa-5:

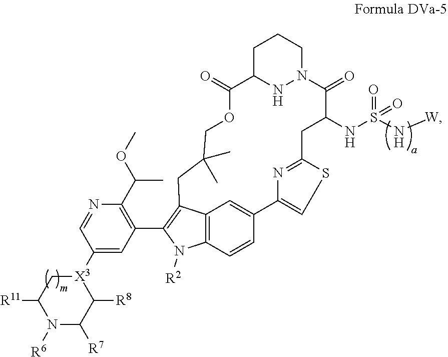

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DVIa:

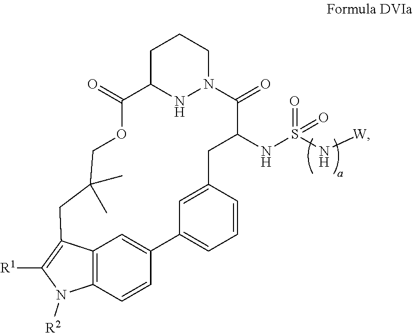

wherein a is 0 or 1.

In some embodiments, the RAS(ON) inhibitor, or a pharmaceutically acceptable salt thereof, has the structure of Formula DVIa-1:

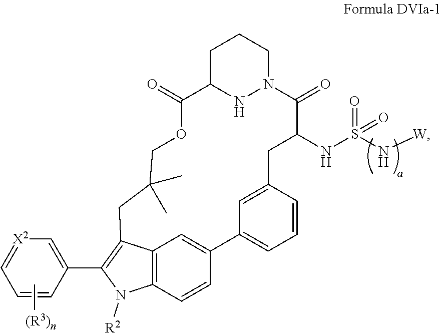

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVIa-2:

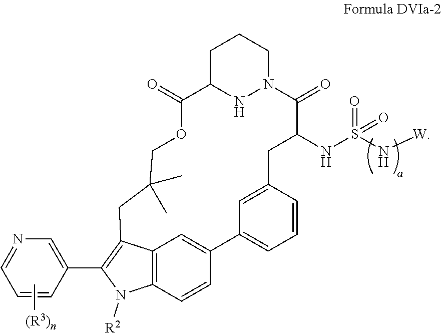

In some embodiments, the RAS(ON) inhibitor, or a pharmaceutically acceptable salt thereof, has the structure of Formula DVIa-3:

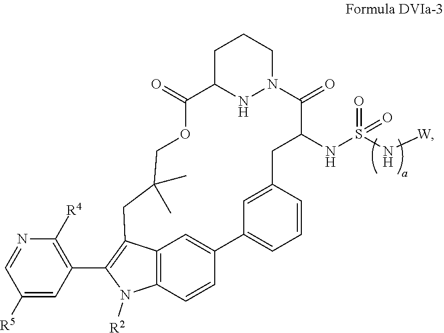

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVIa-4:

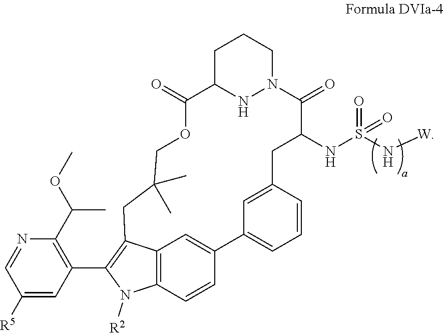

In some embodiments, the RAS(ON) inhibitor, or a pharmaceutically acceptable salt thereof, has the structure of Formula DVIa-5:

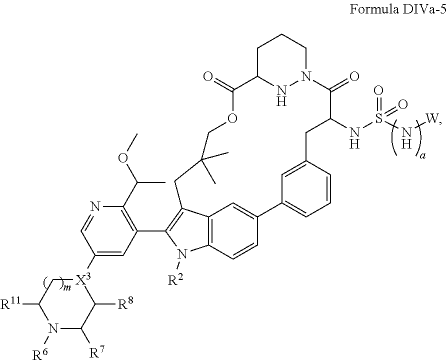

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DVIIa:

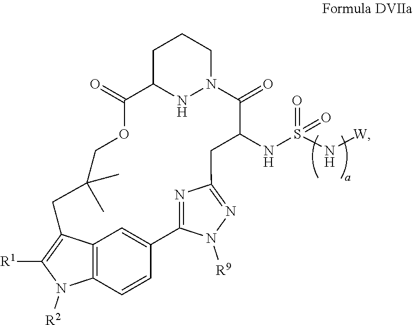

wherein R⁹ is H or C₁-C₆ alkyl; and
a is 0 or 1.

In some embodiments, the RAS(ON) inhibitor, or a pharmaceutically acceptable salt thereof, has the structure of Formula DVIIa-1

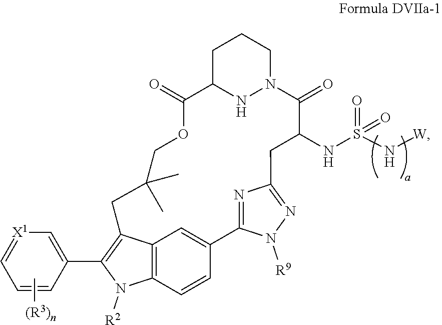

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVIIa-2:

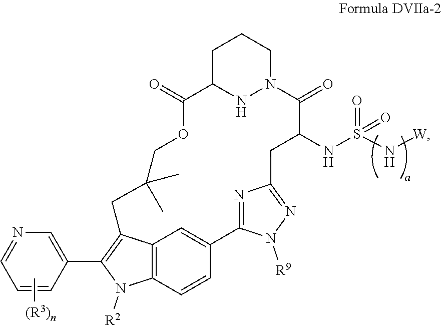

In some embodiments, the RAS(ON) inhibitor, or a pharmaceutically acceptable salt thereof, has the structure of Formula DVIIa-3:

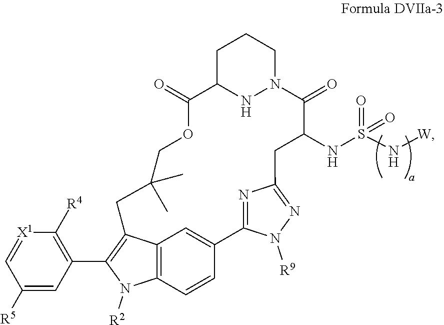

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVIIa-4:

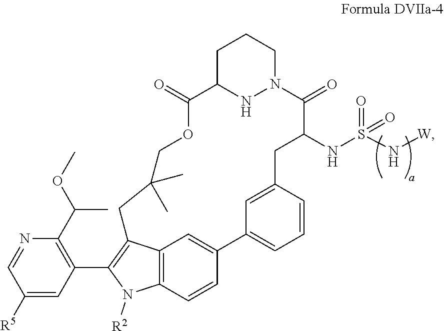

In some embodiments, the RAS(ON) inhibitor, or a pharmaceutically acceptable salt thereof, has the structure of Formula DVIIa-5:

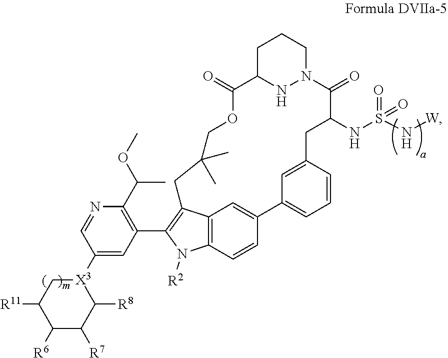

wherein X³ is N or CH;
m is 1 or 2;
R⁶, R⁷, R⁸, and R¹¹ are each independently selected from hydrogren, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae DVIIa, DVIIa-1, DVIIa-2, DVIIa-3, DVIIa-4, or DVIIa-5), R⁹ is methyl.

In some embodiments, Y is —NHS(O)— or —NHS(O)NH—.

In some embodiments, the RAS(ON) inhibitor, or a pharmaceutically acceptable salt thereof, has the structure of Formula DVIIIa:

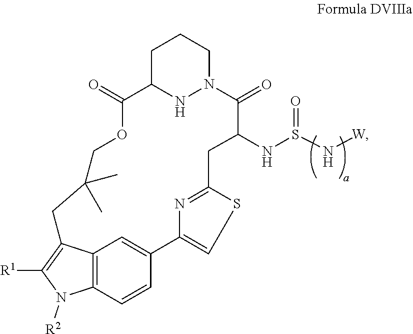

wherein a is 0 or 1.

In some embodiments, the RAS(ON) inhibitor, or a pharmaceutically acceptable salt thereof, has the structure of Formula Villa-1:

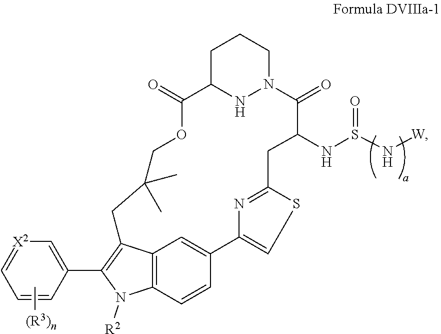

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVIIIa-2:

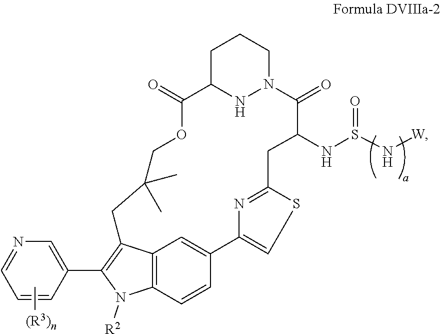

In some embodiments, the RAS(ON) inhibitor, or a pharmaceutically acceptable salt thereof, has the structure of Formula DVIIIa-3:

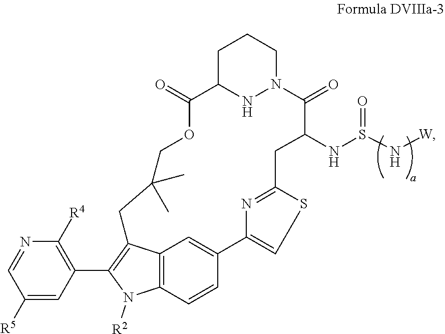

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVIIIa-4:

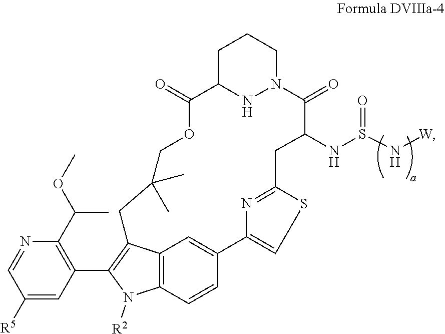

In some embodiments, the RAS(ON) inhibitor, or a pharmaceutically acceptable salt thereof, has the structure of Formula DVIIIa-5:

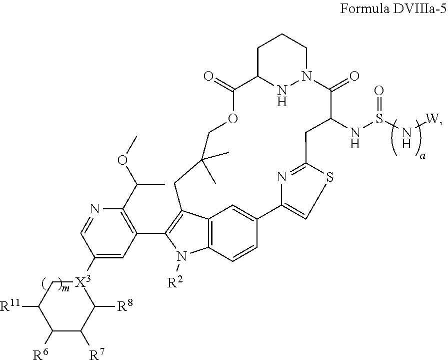

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DIXa:

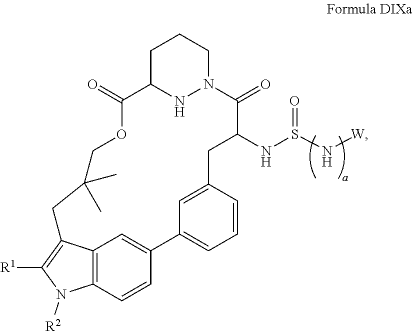

wherein a is 0 or 1.

In some embodiments, the RAS(ON) inhibitor, or a pharmaceutically acceptable salt thereof, has the structure of Formula DIXa-1:

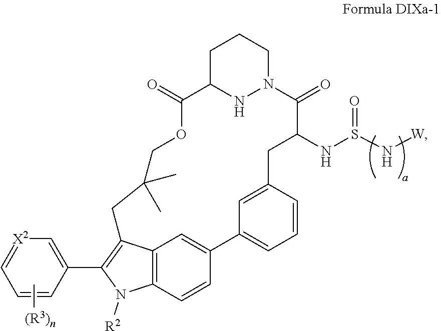

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIXa-2:

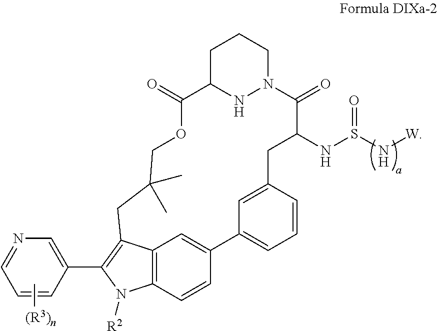

In some embodiments, the RAS(ON) inhibitor, or a pharmaceutically acceptable salt thereof, has the structure of Formula DIXa-3:

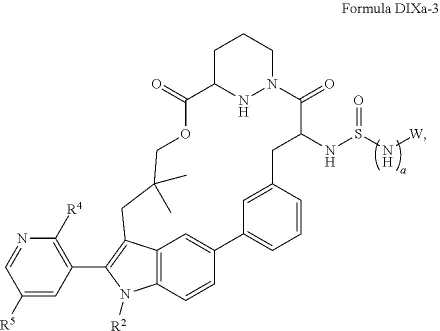

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIXa-4:

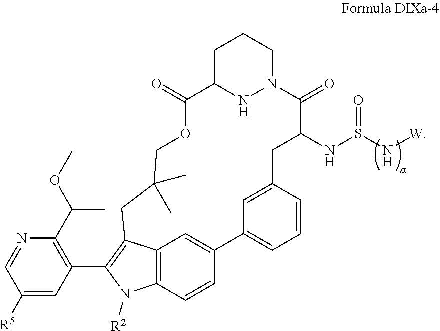

In some embodiments, the RAS(ON) inhibitor, or a pharmaceutically acceptable salt thereof, has the structure of Formula DIXa-5:

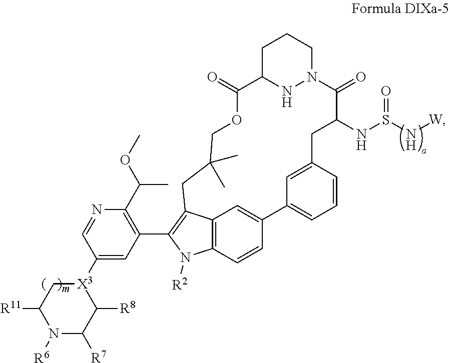

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
   R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DXa:

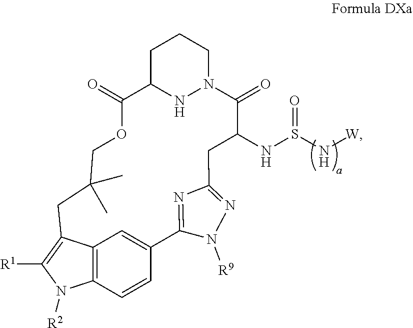

wherein R⁹ is H or C₁-C₆ alkyl; and
a is 0 or 1.

In some embodiments, the RAS(ON) inhibitor, or a pharmaceutically acceptable salt thereof, has the structure of Formula DXa-1:

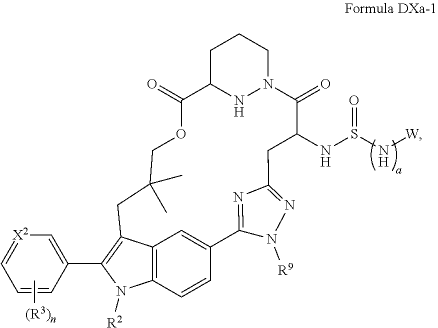

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DXa-2:

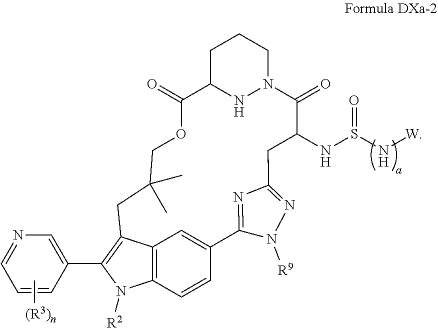

In some embodiments, the RAS(ON) inhibitor, or a pharmaceutically acceptable salt thereof, has the structure of Formula DXa-3:

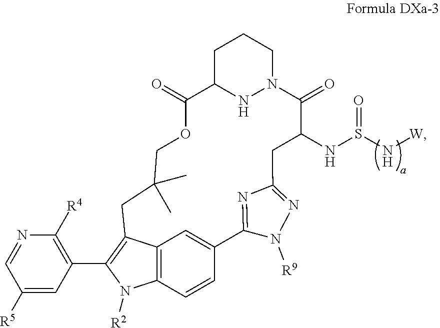

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DXa-4:

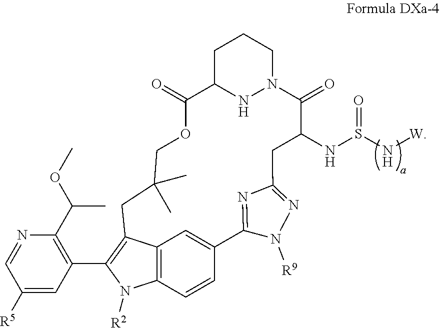

In some embodiments, the RAS(ON) inhibitor, or a pharmaceutically acceptable salt thereof, has the structure of Formula DXa-5:

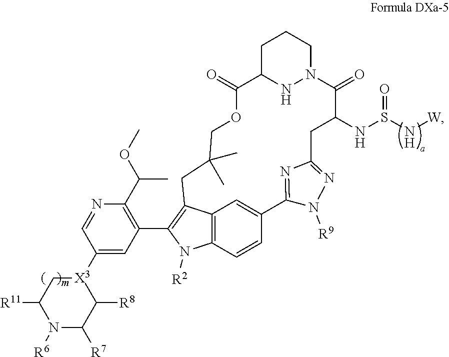

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae DXa, DXa-1, DXa-2, DXa-3, DXa-4, or DXa-5), R⁹ is methyl.

In some embodiments of formula DIa or subformula thereof, a is 0. In some embodiments of formula DIa or subformula thereof, a is 0.

In some embodiments of formula DIa or subformula thereof, R² is optionally substituted C₁-C₆ alkyl. In some embodiments, R² is selected from —CH₂CH₃ or —CH₂CF₃.

In some embodiments of formula DIa or subformula thereof, W is C₁-C₄ alkyl. In some embodiments, W is:

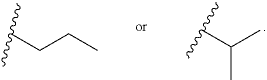

In some embodiments of formula DIa or subformula thereof, W is optionally substituted cyclopropyl, optionally substituted cyclobutyl, optionally substituted cyclopentyl, or optionally substituted cyclohexyl, optionally substituted piperidine, optionally substituted piperazine, optionally substituted pyridine, or optionally substituted phenyl.

In some embodiments of formula DIa or subformula thereof, W is optionally substituted 3 to 10-membered heterocycloalkyl, optionally substituted 3 to 10-membered cycloalkyl, optionally substituted 6 to 10-membered aryl, or optionally substituted 5 to 10-membered heteroaryl.

In some embodiments of formula DIa or subformula thereof, W is optionally substituted 3 to 10-membered heterocycloalkyl. In some embodiments, W is selected from the following, or a stereoisomer thereof:

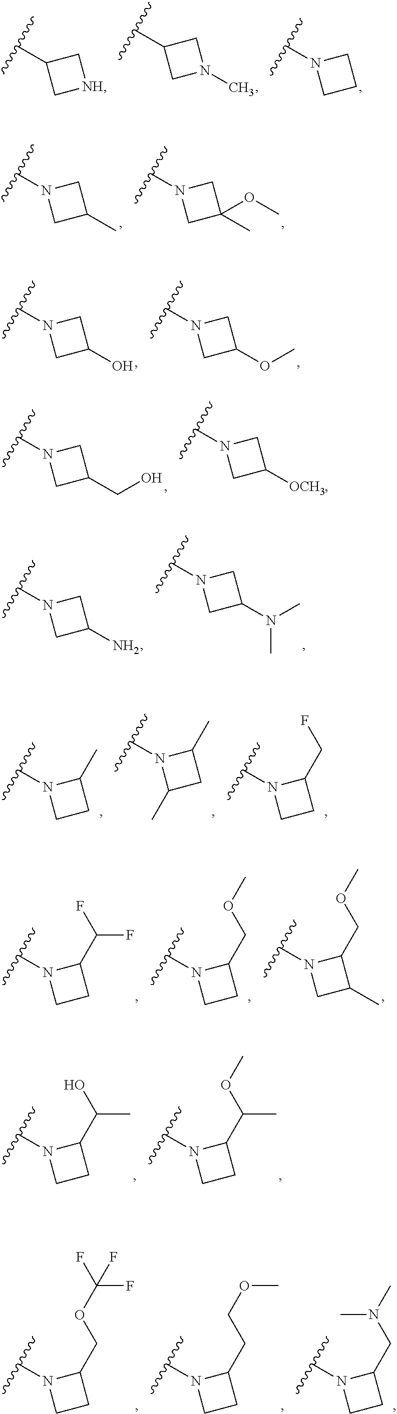

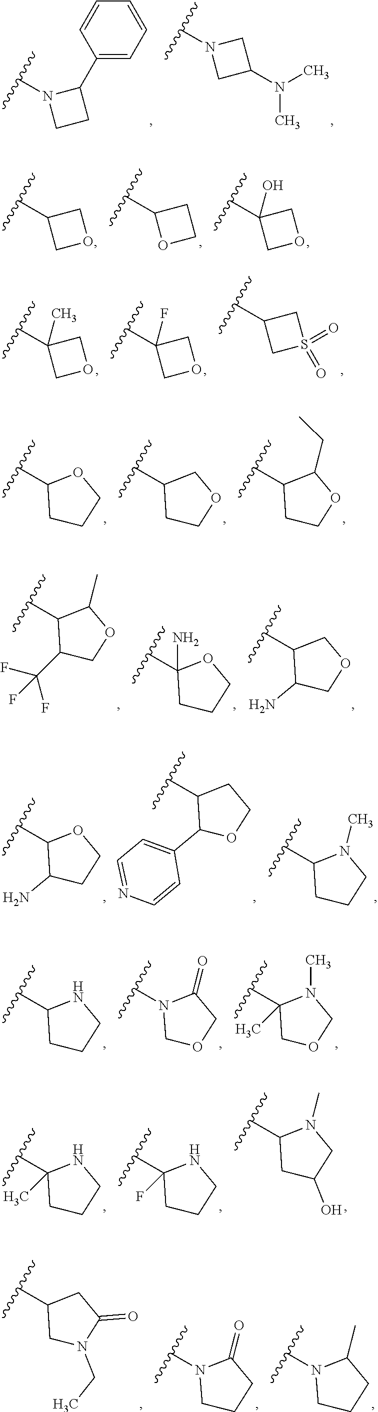

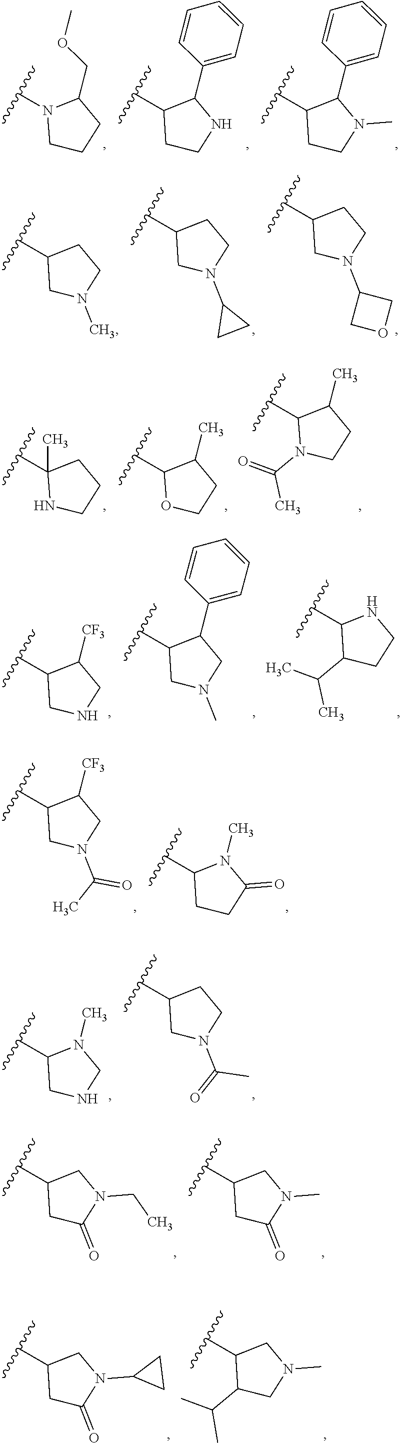

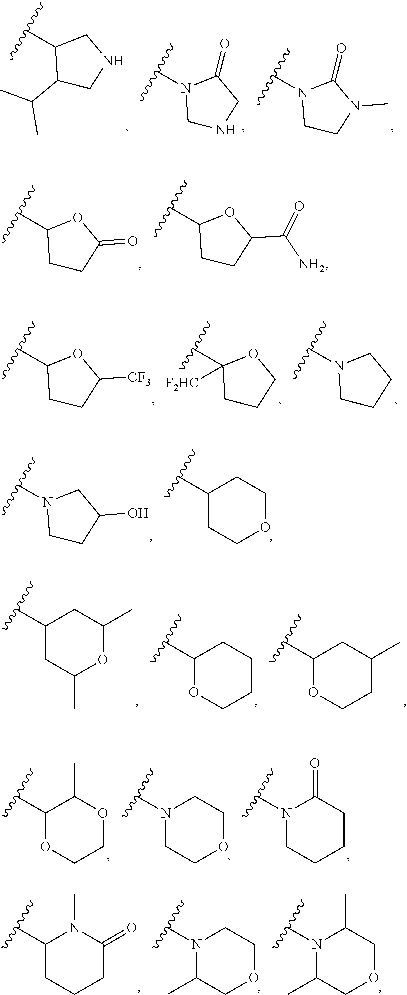

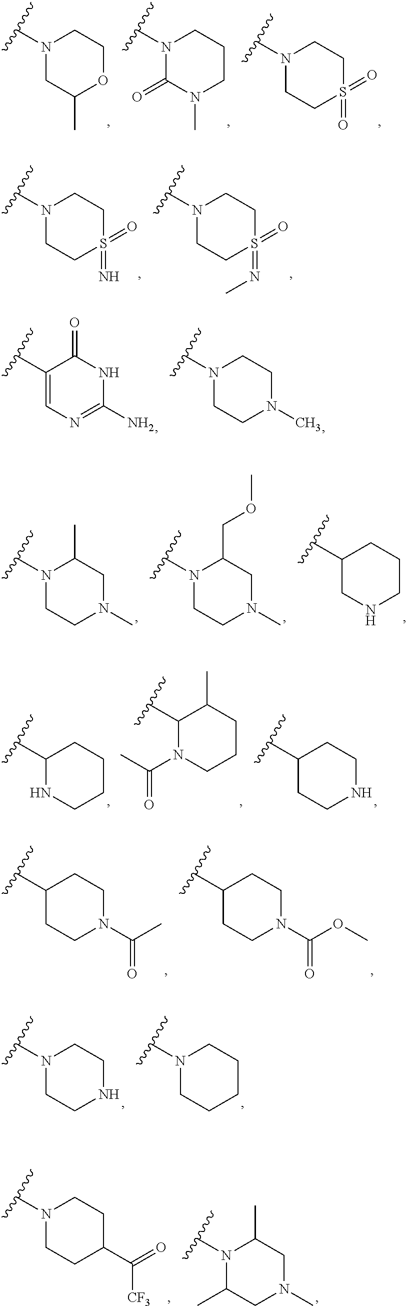

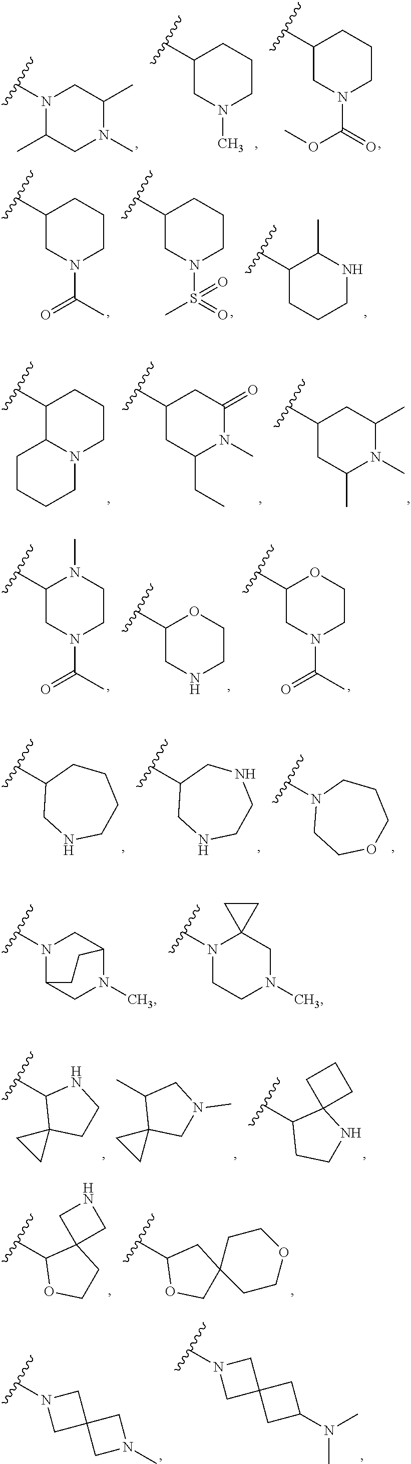

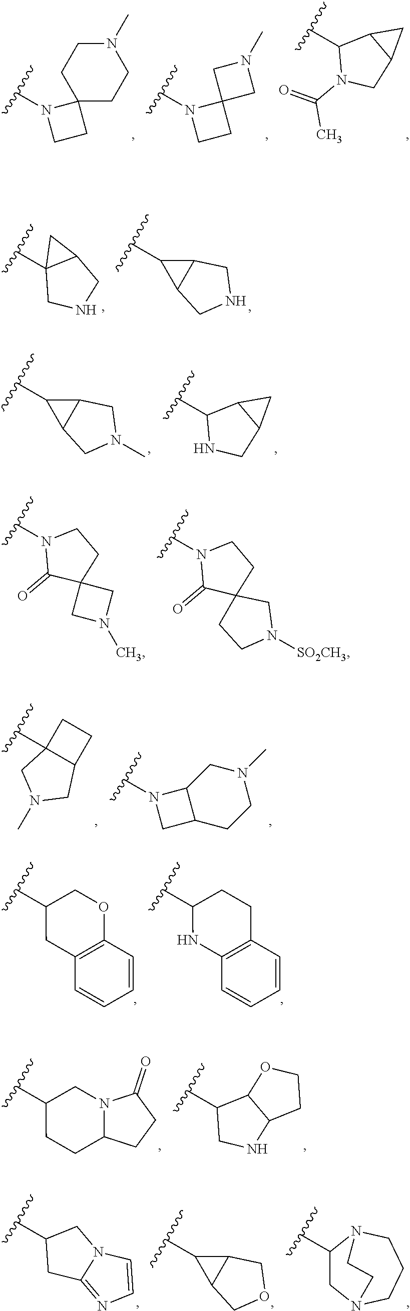

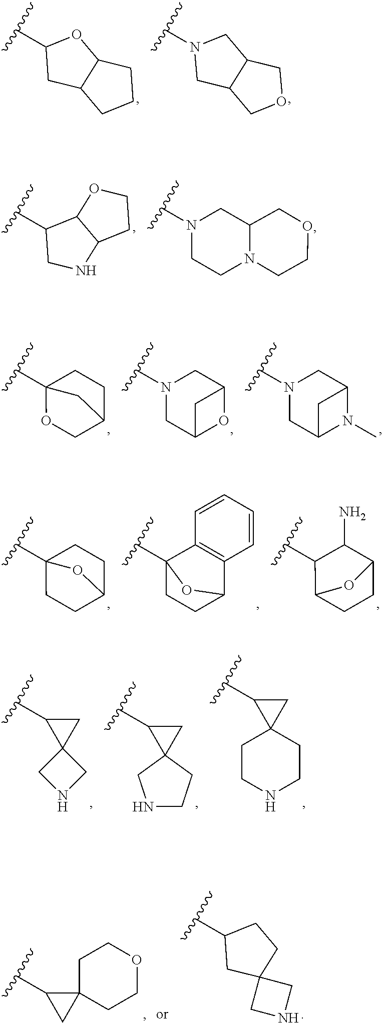

In some embodiments, W is selected from the following, or a stereoisomer thereof:

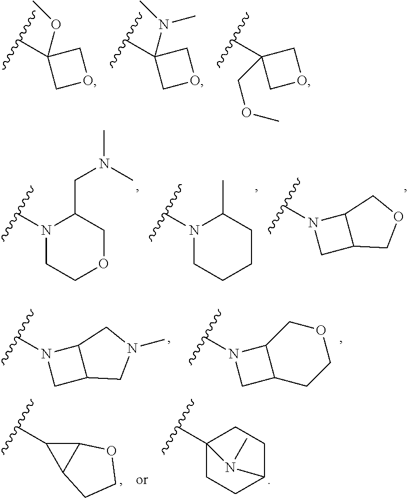

In some embodiments of formula DIa or subformula thereof, W is optionally substituted 3 to 10-membered cycloalkyl. In some embodiments, W is selected from the following, or a stereoisomer thereof:

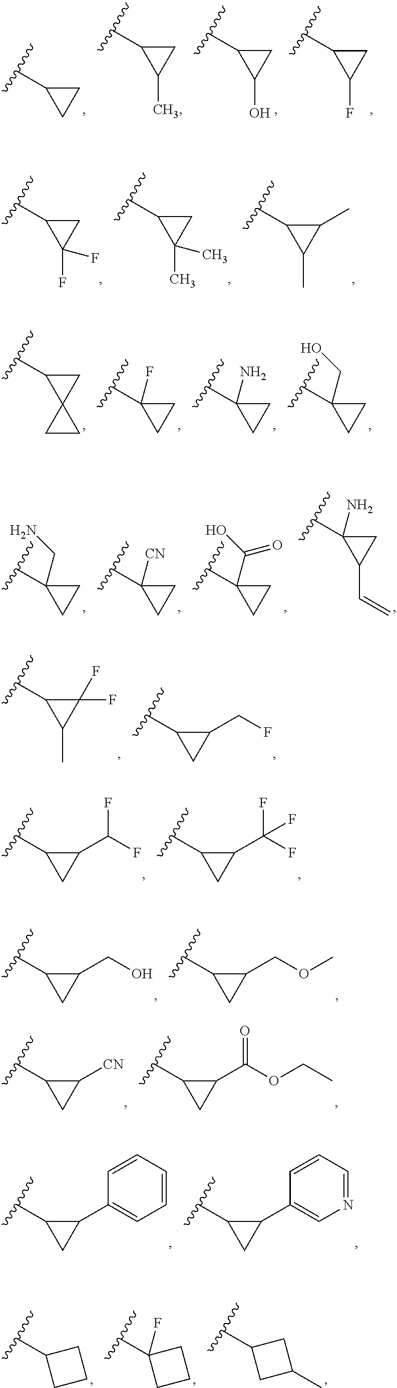

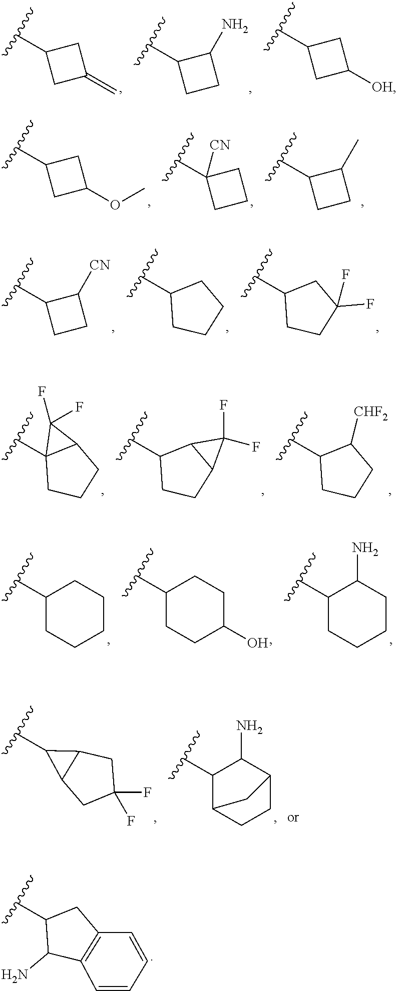

In some embodiments, W is selected from the following, or a stereoisomer thereof:

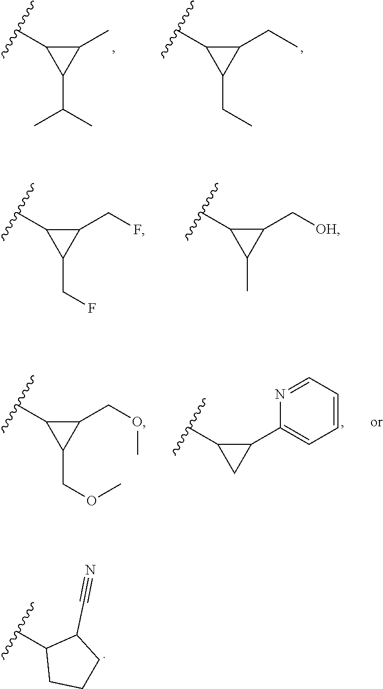

In some embodiments of formula DIa or subformula thereof, W is optionally substituted 5 to 10-membered heteroaryl. In some embodiments, W is selected from the following, or a stereoisomer thereof:

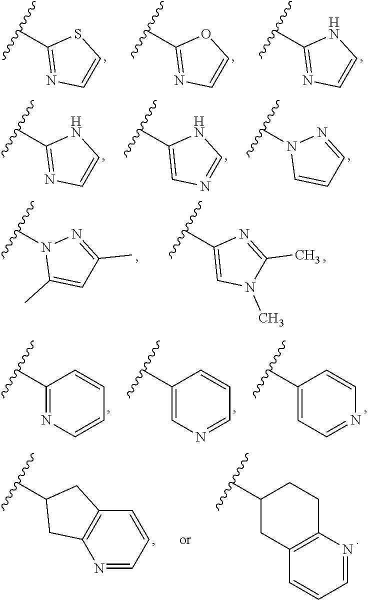

In some embodiments of formula DIa or subformula thereof, W is optionally substituted 6 to 10-membered aryl. In some embodiments, W is optionally substituted phenyl.

In some embodiments of formula DIa or subformula thereof, W is optionally substituted C₁-C₃ heteroalkyl. In some embodiments, W is selected from the following, or a stereoisomer thereof:

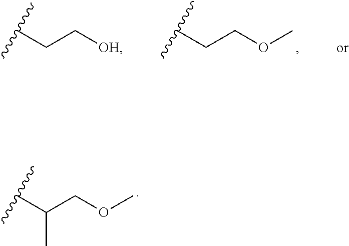

In some embodiments, the RAS(ON) inhibitor, or pharmaceutically acceptable salt thereof, has the structure of Formula Dib:

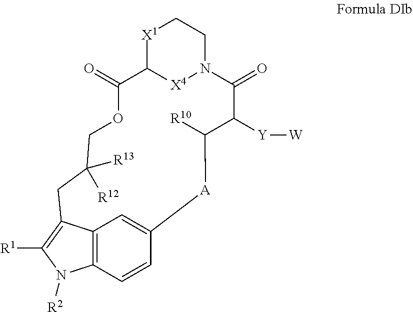

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;

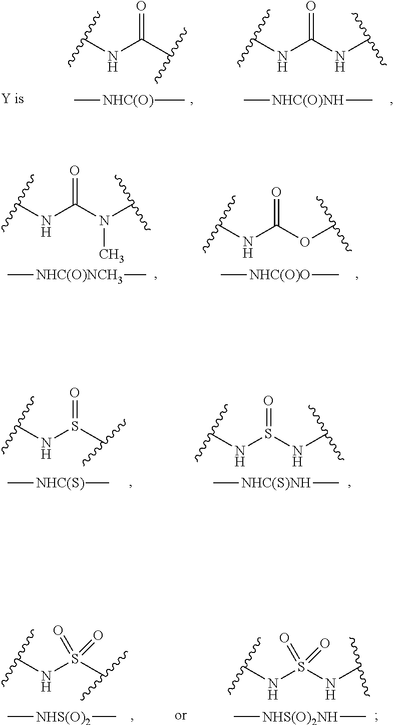

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl; or W is —R¹⁴C(═O)R¹⁵ where R¹⁴ is 3 to 10-membered cycloalkylene and R¹⁵ is selected from optionally substituted 3 to 10-membered cycloalkyl, optionally substituted 6 to 10-membered aryl, or optionally substituted 5 to 10-membered heteroaryl;
X¹ and X⁴ are each, independently, CH₂, CH(CH₃) or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3 to 7-membered heterocycloalkyl; and
R¹² and R¹³ are each, independently, selected from F or CH₃, or R¹² and R¹³ combine with the atoms to which they are attached to make a 3-membered cycloalkyl

In some embodiments, the RAS(ON) inhibitor is selected from Table D1a, or a pharmaceutically acceptable salt or stereoisomer thereof. In some embodiments, the RAS(ON) inhibitor is selected from Table D1a, or a pharmaceutically acceptable salt or atropisomer thereof.

TABLE D1a
Certain Compounds of the Present Invention

| Ex. # | Structure |
|---|---|
| DA1 | 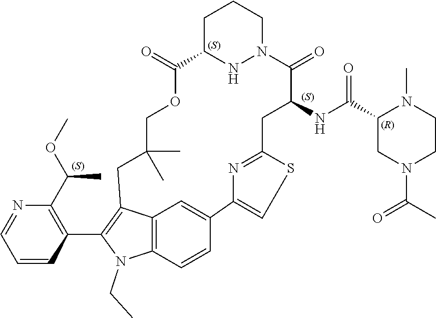 |
| DA2 | 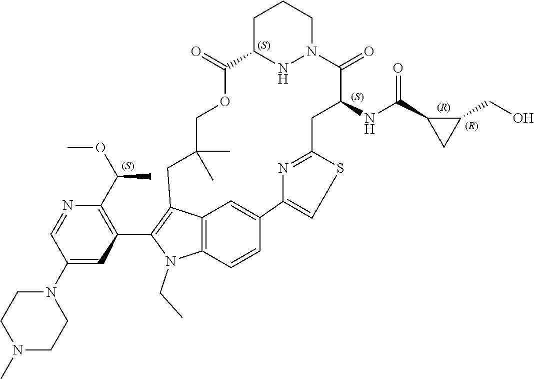 |
| DA3 | 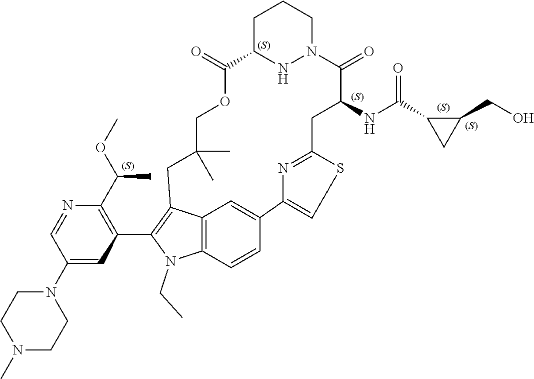 |
| DA4 | 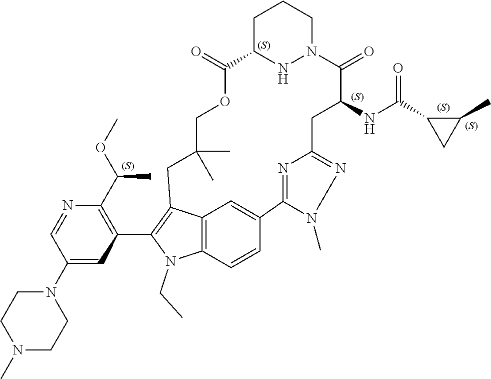 |
| DA5 | 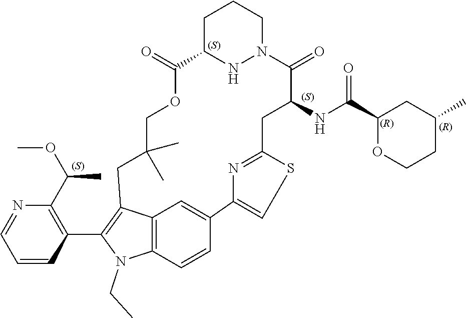 |
| DA6 | 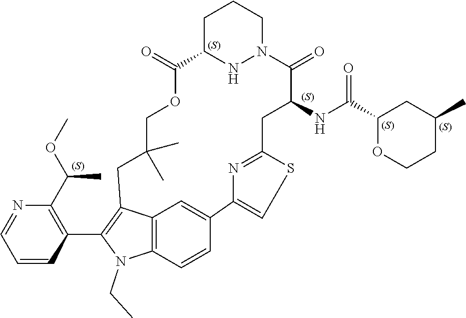 |
| DA7 | 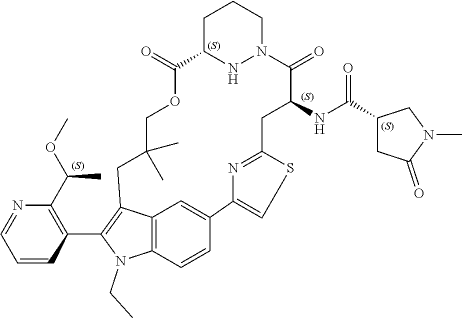 |
| DA8 | 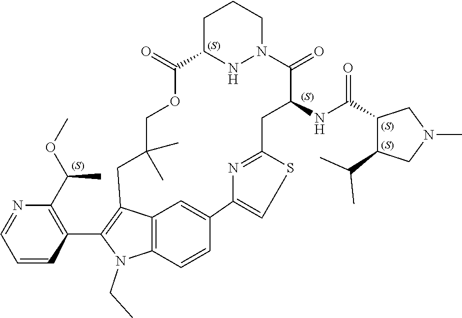 |
| DA9 | 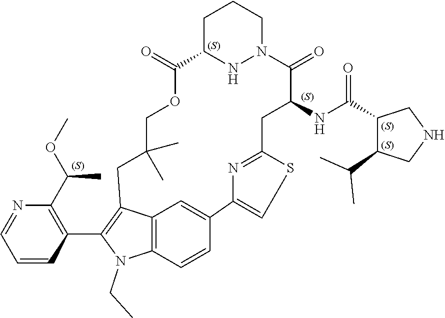 |
| DA10 | 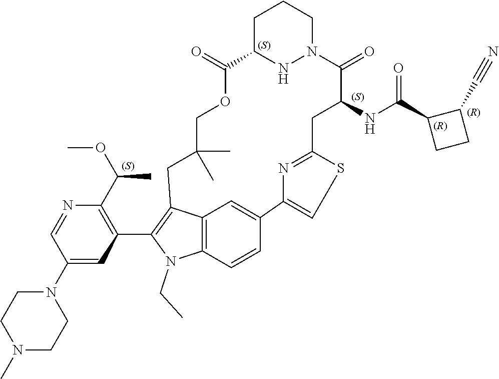 |
| DA11 | 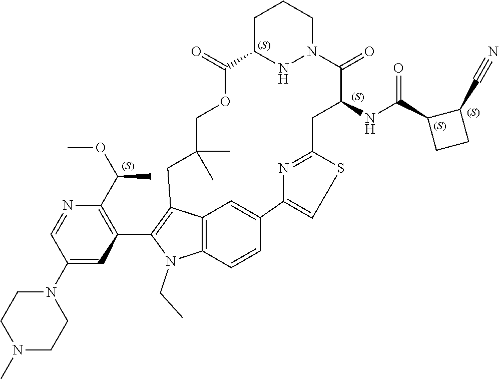 |
| DA12 | 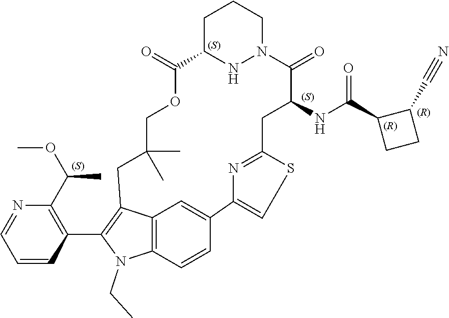 |
| DA13 | 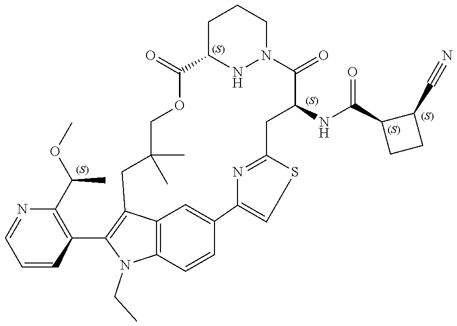 |
| DA14 | 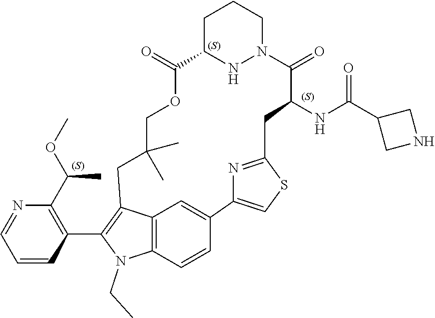 |
| DA15 | 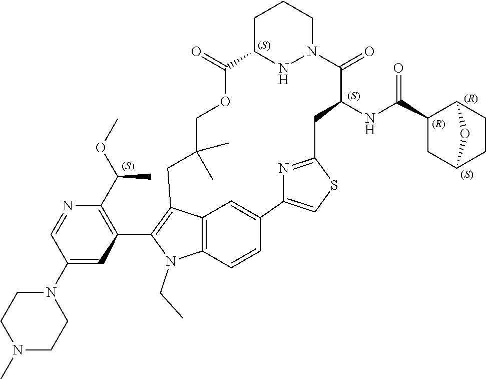 |
| DA16 | 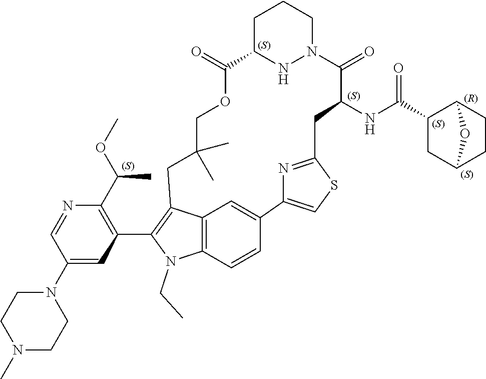 |
| DA17 | 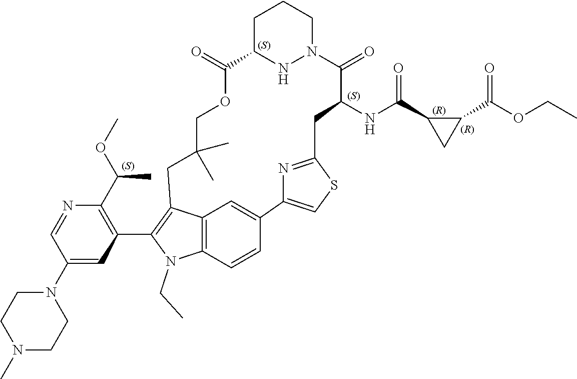 |
| DA18 | 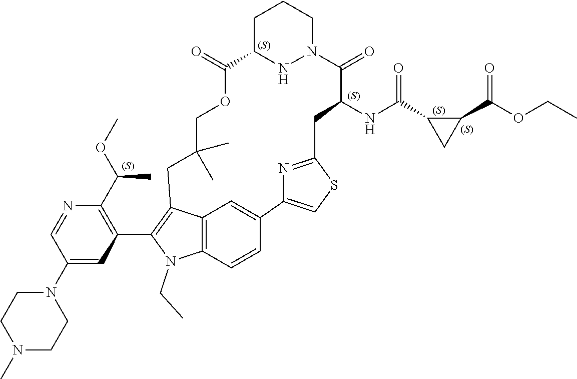 |
| DA19 | 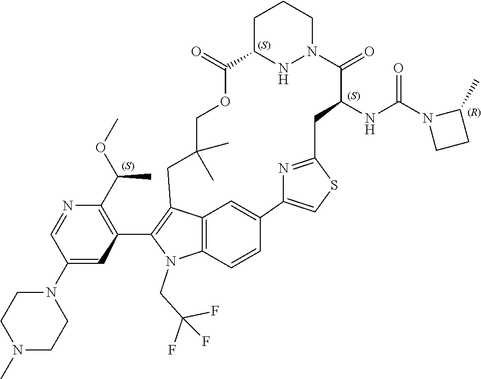 |
| DA20 | 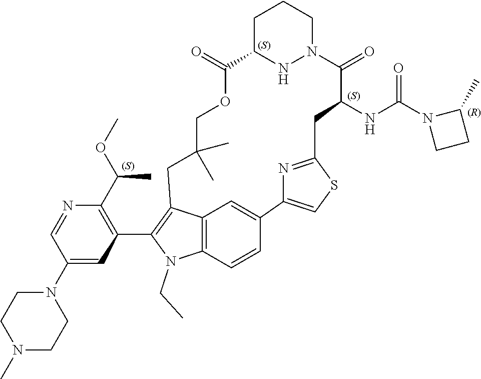 |
| DA21 | 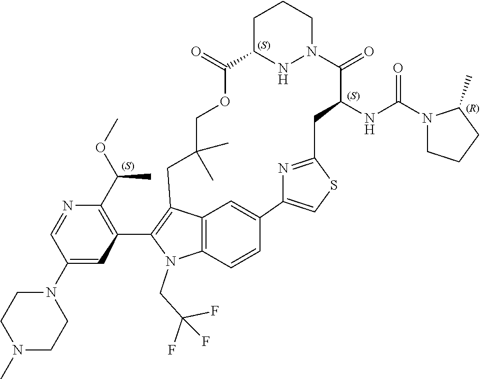 |
| DA22 | 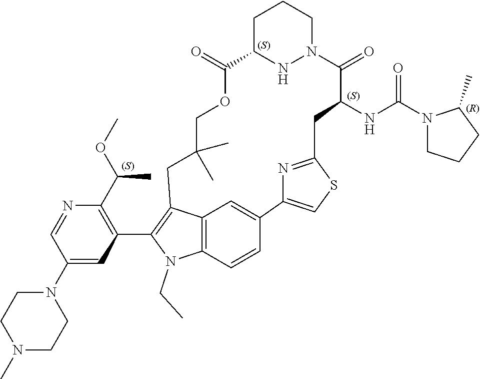 |
| DA23 | 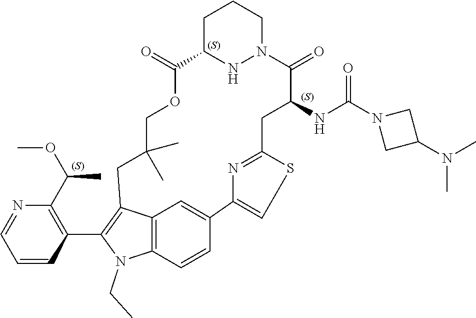 |
| DA24 | 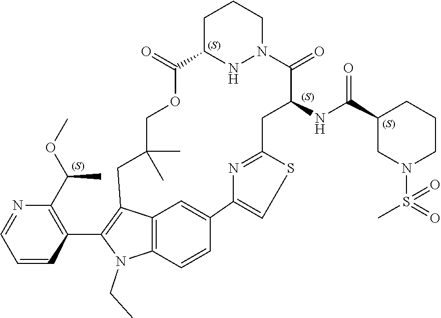 |
| DA25 | 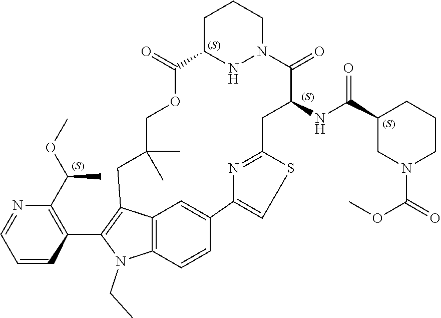 |
| DA26 | 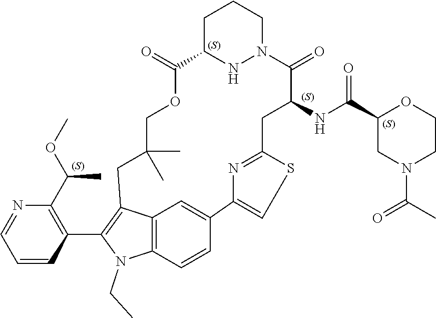 |
| DA27 | 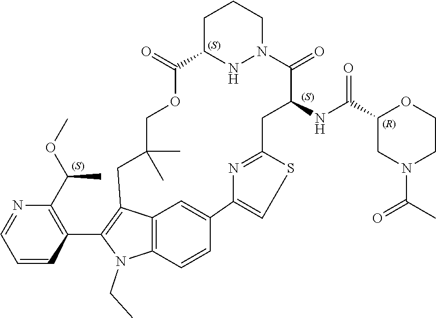 |
| DA28 | 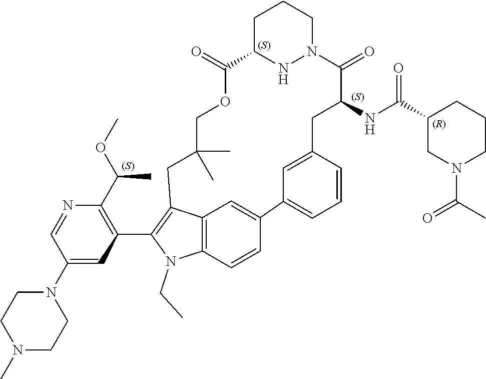 |
| DA29 | 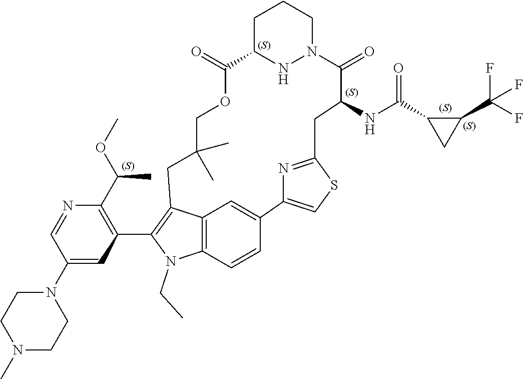 |
| DA30 | 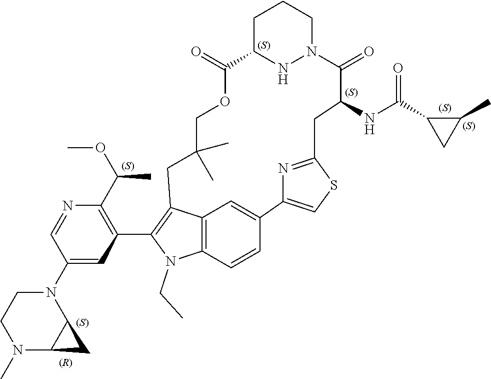 |
| DA31 | 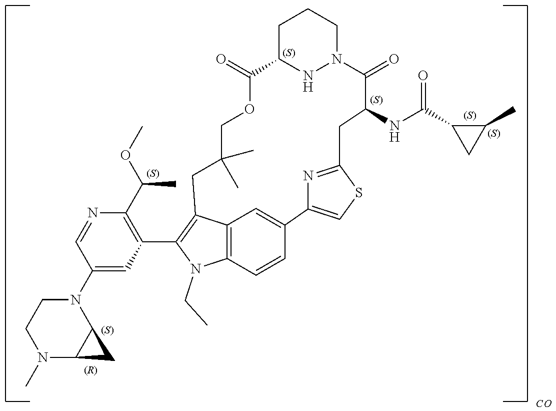 |
| DA32 | 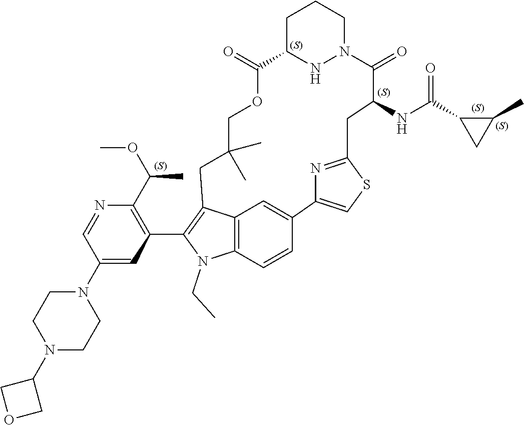 |
| DA33 | 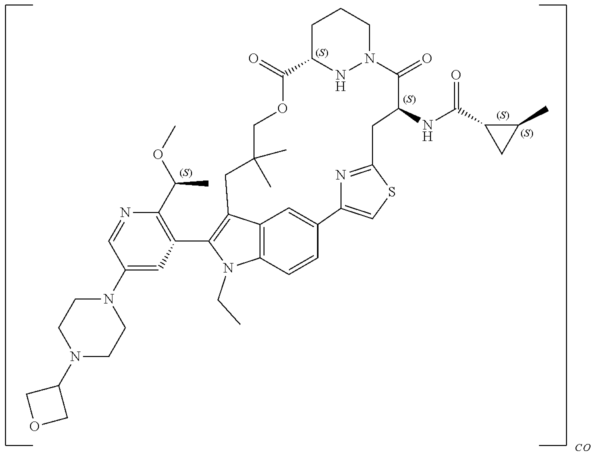 |
| DA34 | 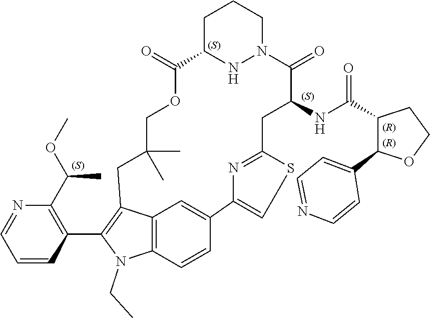 |
| DA35 | 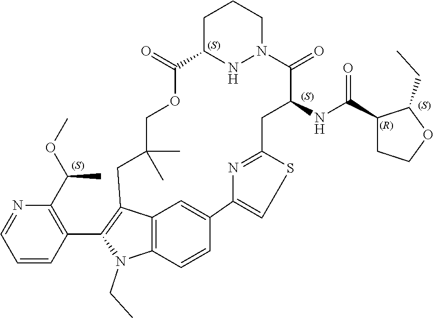 |
| DA36 | 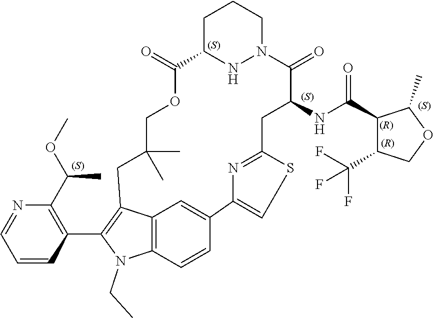 |
| DA37 | 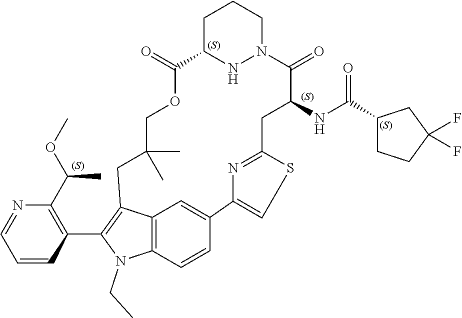 |
| DA38 | 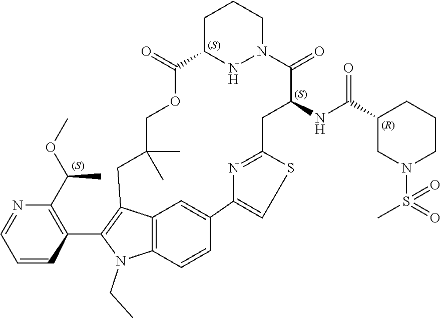 |
| DA39 | 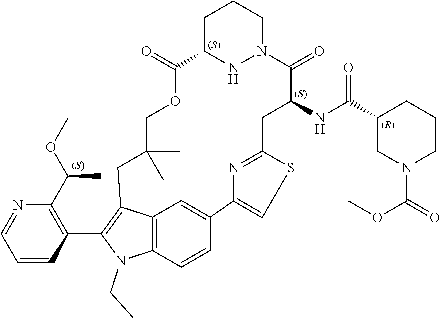 |
| DA40 | 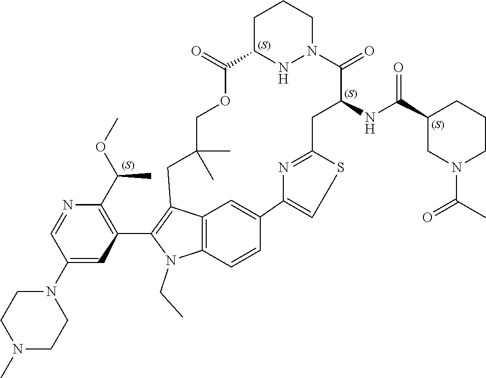 |
| DA41 | 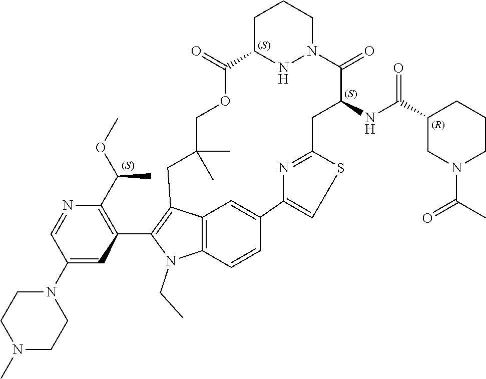 |
| DA42 | 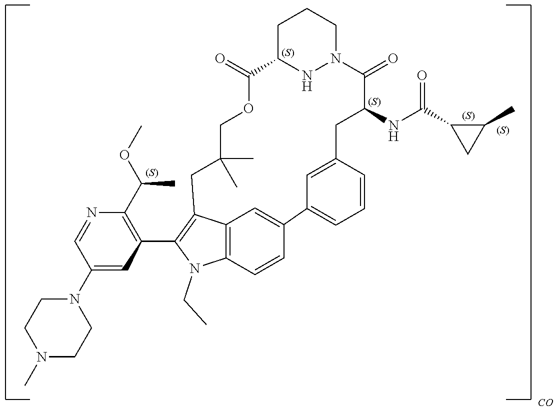 |
| DA43 | 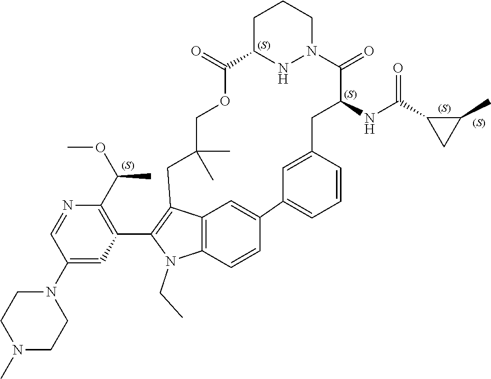 |
| DA44 | 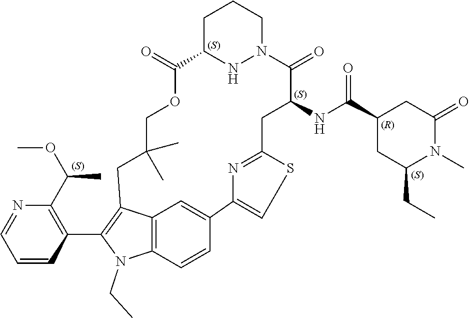 |
| DA45 | 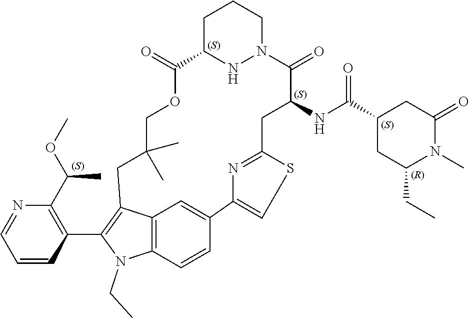 |
| DA46 | 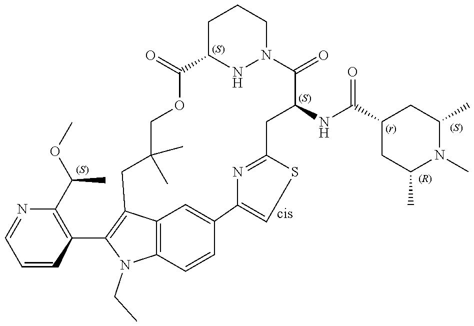 |
| DA47 | 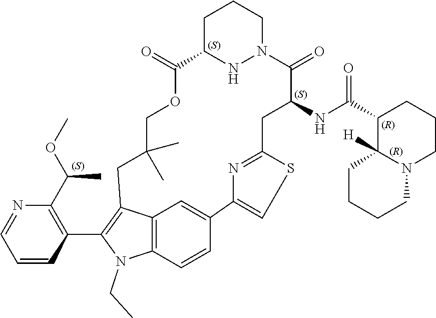 |
| DA48 | 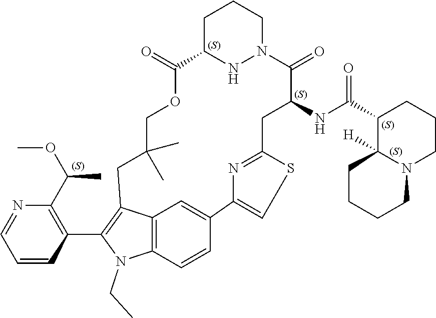 |
| DA49 | 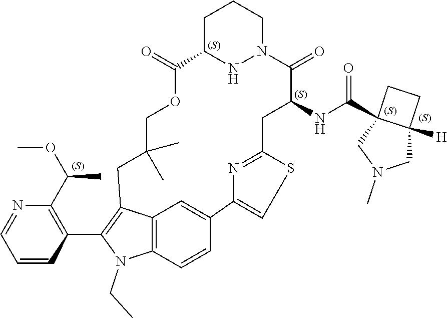 |
| DA50 | 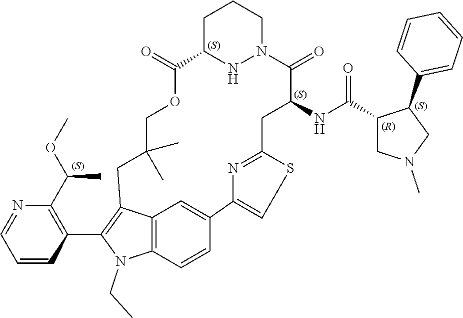 |
| DA51 | 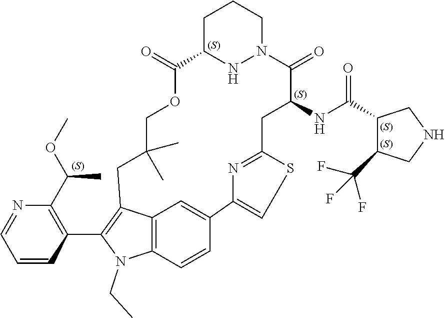 |
| DA52 | 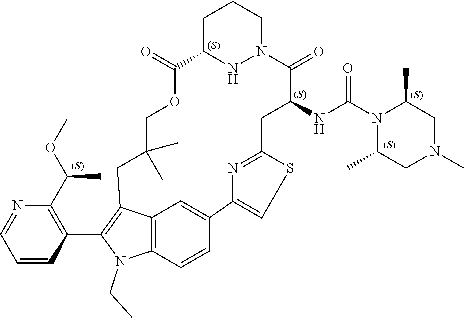 |
| DA53 | 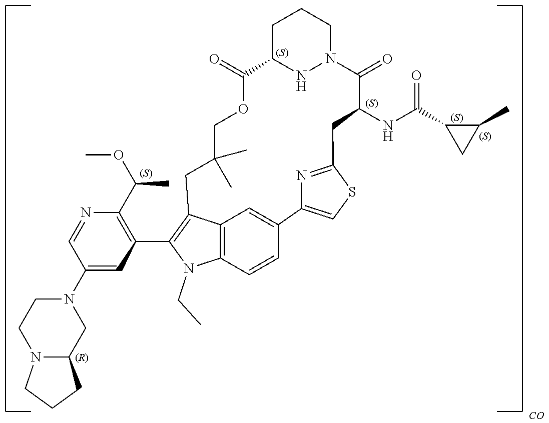 |
| DA54 | 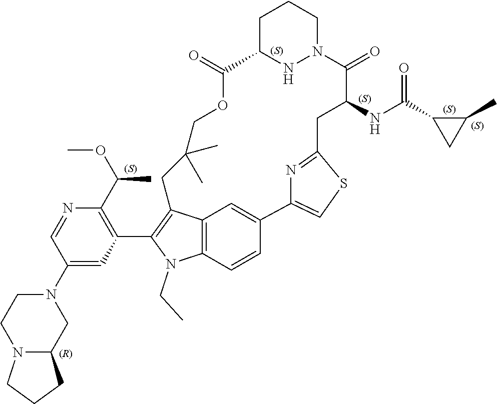 |
| DA55 | 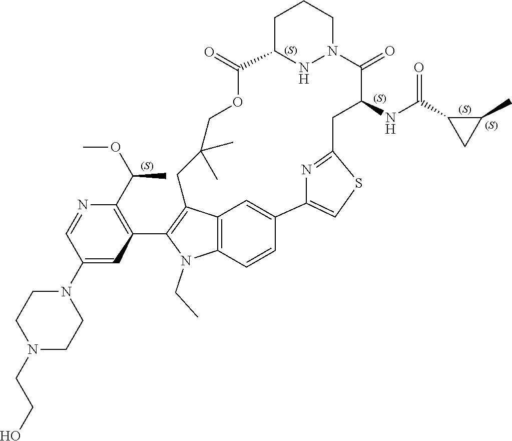 |
| DA56 | 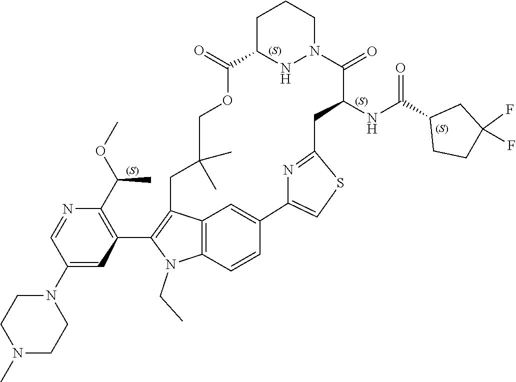 |
| DA57 | 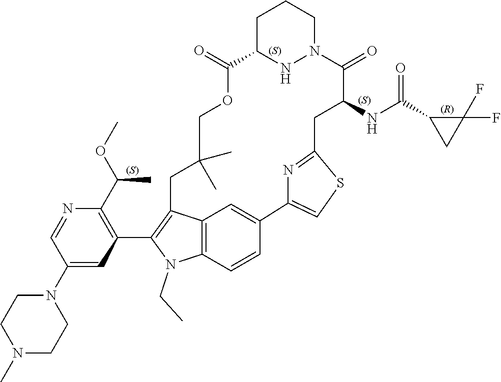 |
| DA58 | 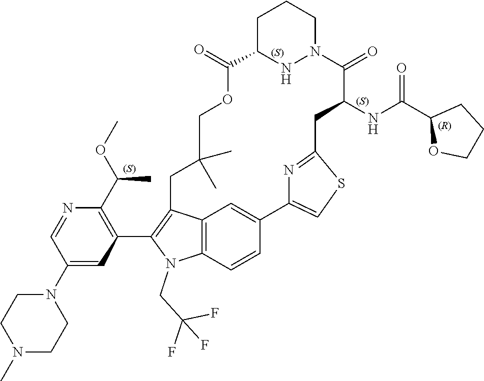 |
| DA59 | 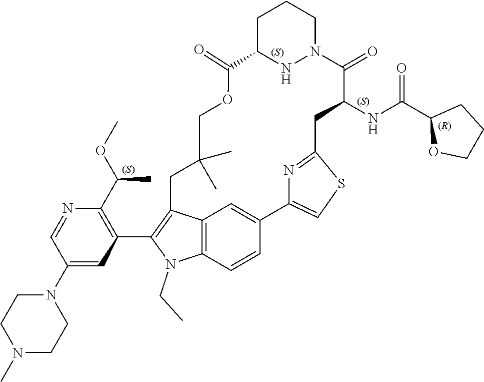 |
| DA60 | 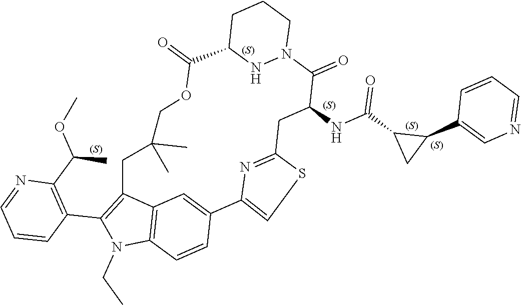 |
| DA61 | 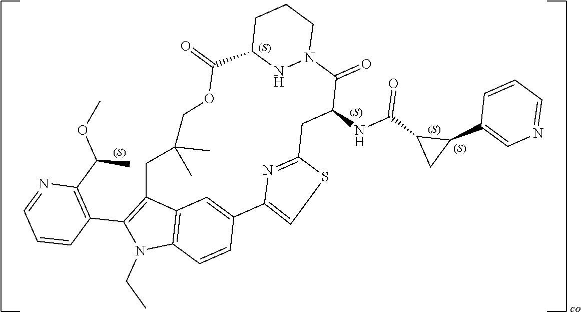 |
| DA62 | 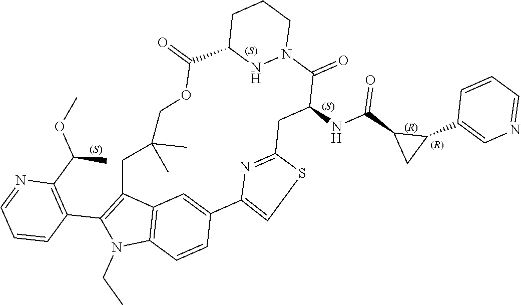 |
| DA63 | 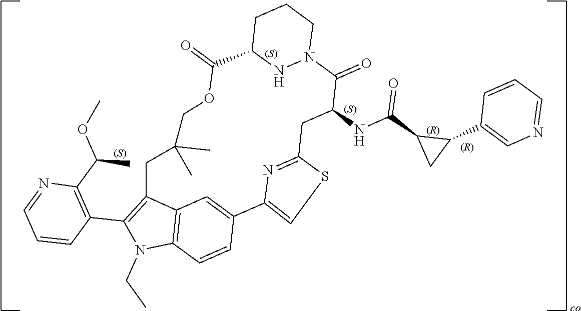 |
| DA64 | 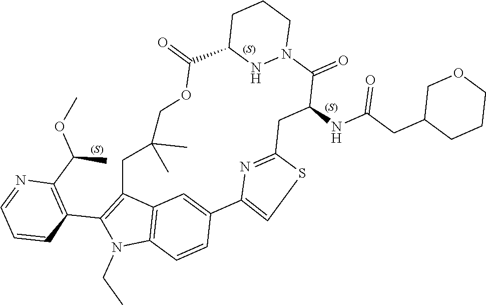 |
| DA65 | 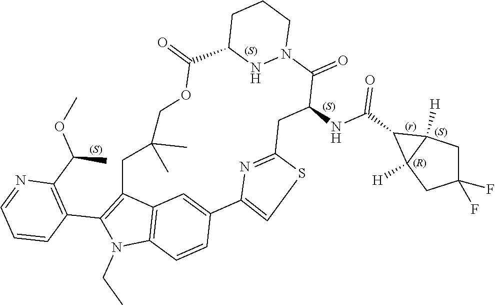 |
| DA66 | 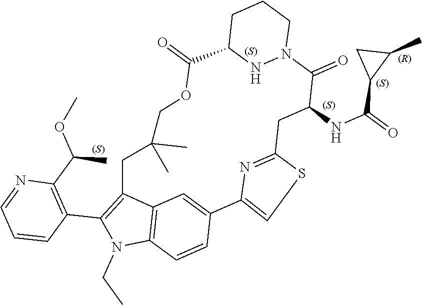 |
| DA67 | 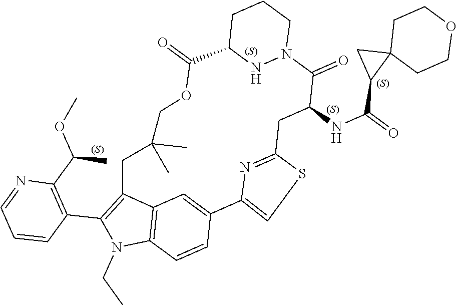 |
| DA68 | 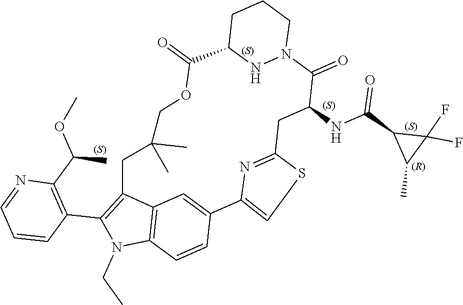 |
| DA69 | 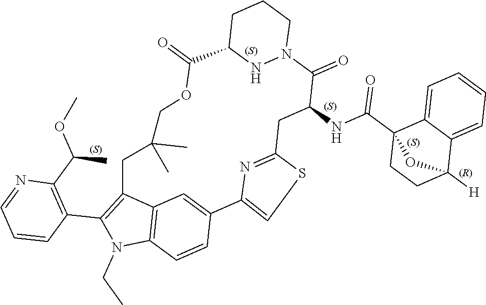 |
| DA70 | 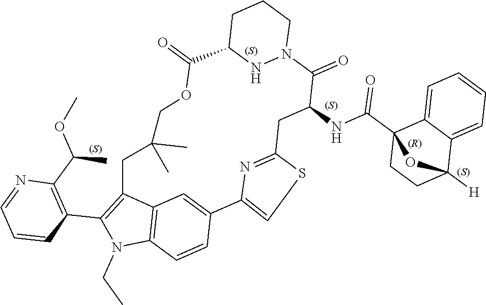 |
| DA71 | 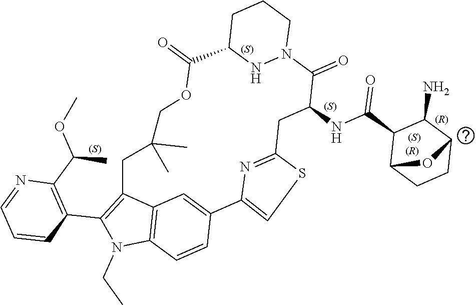 |
| DA72 | 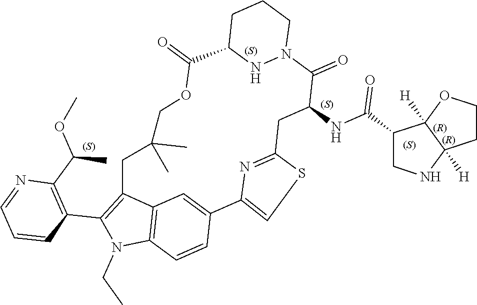 |
| DA73 | 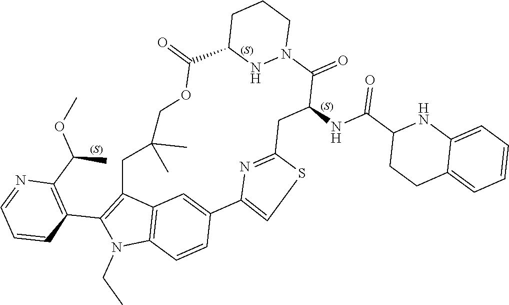 |
| DA74 | 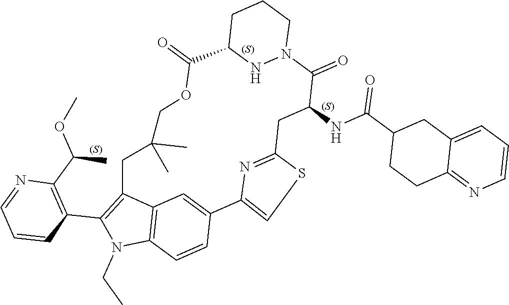 |
| DA75 | 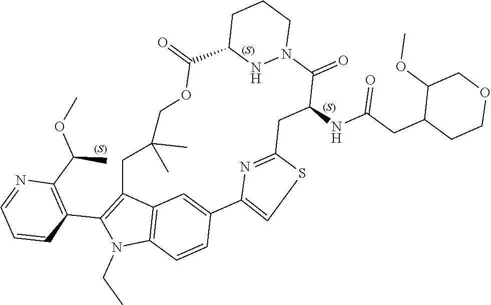 |
| DA76 | 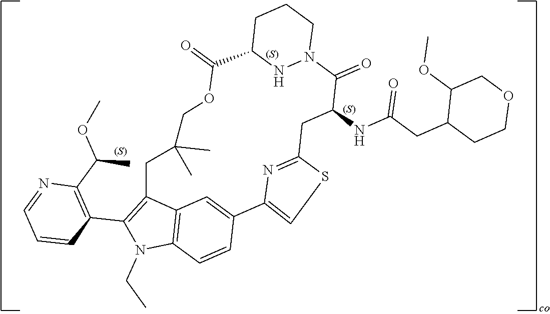 |
| DA77 | 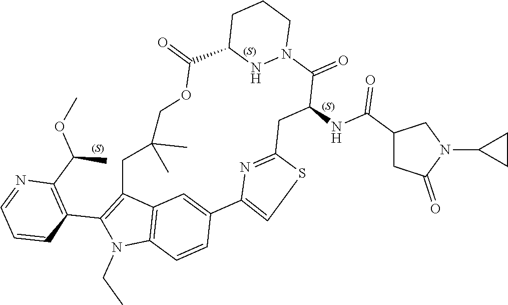 |
| DA78 | 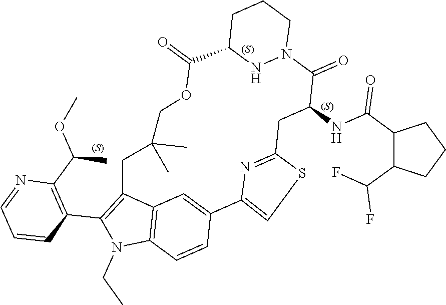 |
| DA79 | 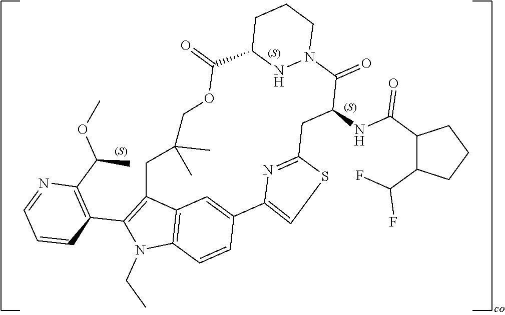 |
| DA80 | 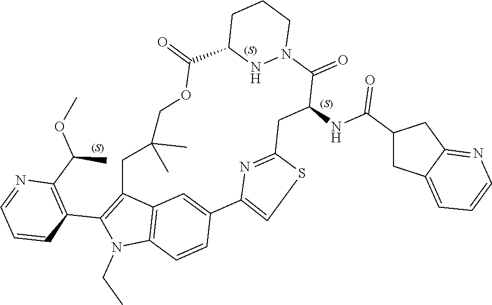 |
| DA81 | 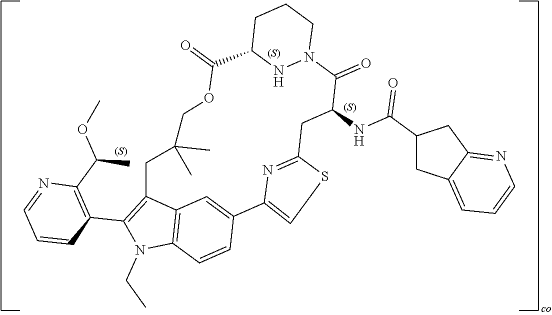 |
| DA82 | 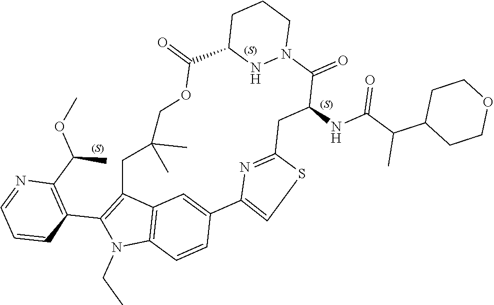 |
| DA83 | 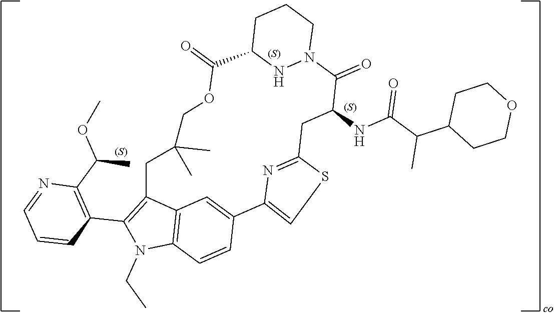 |
| DA84 | 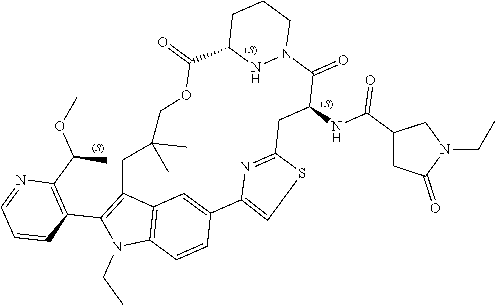 |
| DA85 | 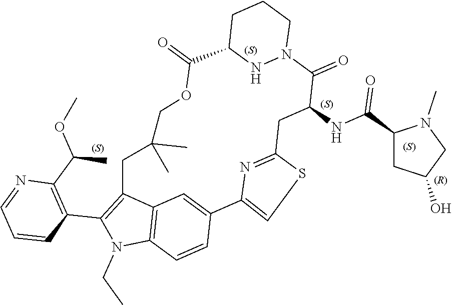 |
| DA86 | 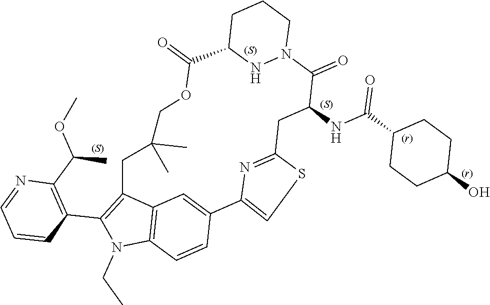 |
| DA87 | 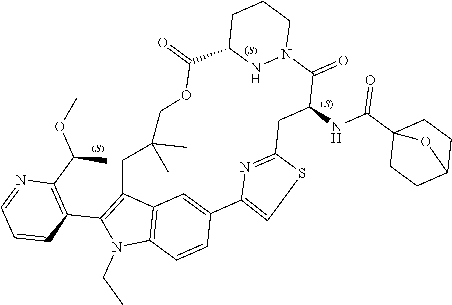 |
| DA88 | 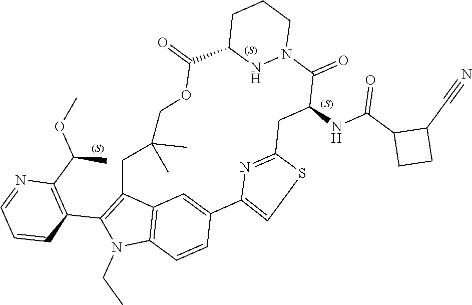 |
| DA89 | 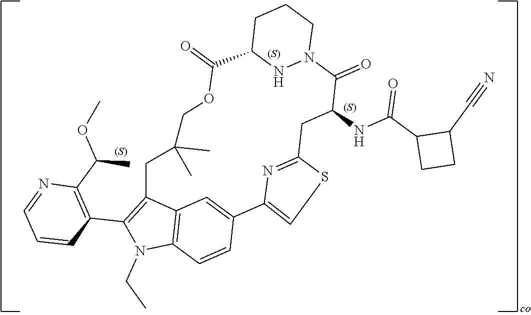 |
| DA90 | 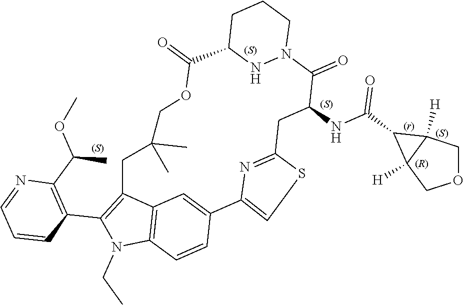 |
| DA91 | 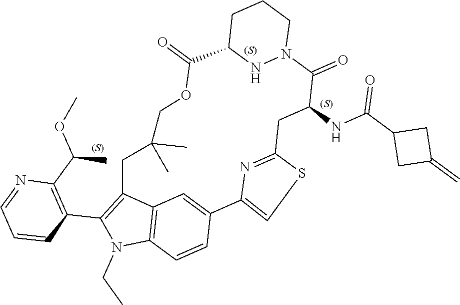 |
| DA92 | 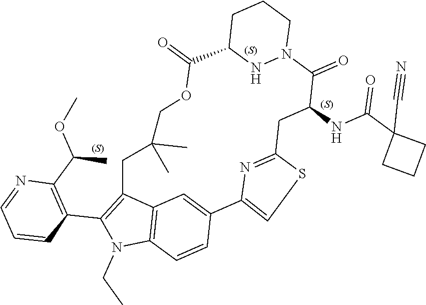 |
| DA93 | 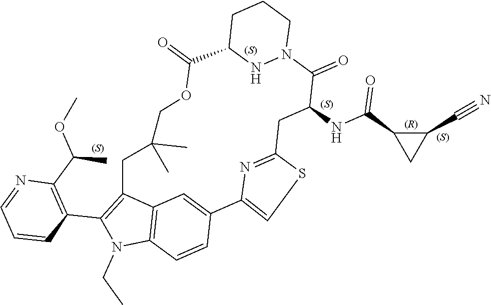 |
| DA94 | 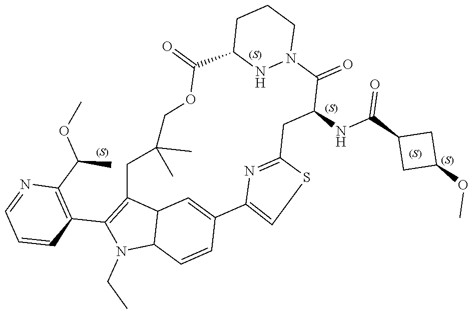 |
| DA95 | 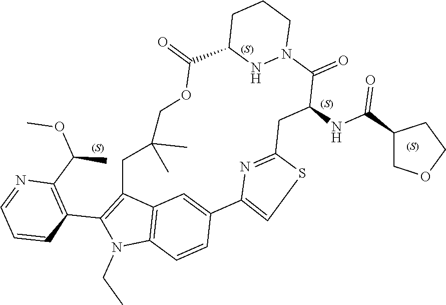 |
| DA96 | 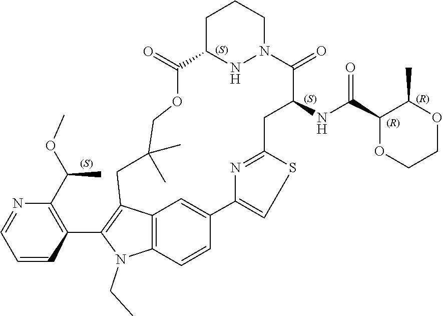 |
| DA97 | 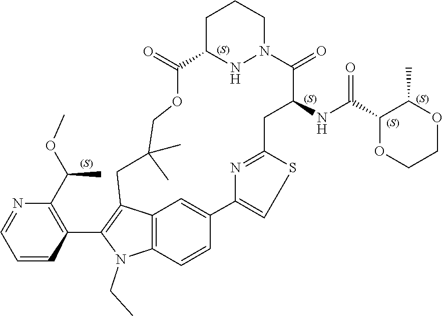 |
| DA98 | 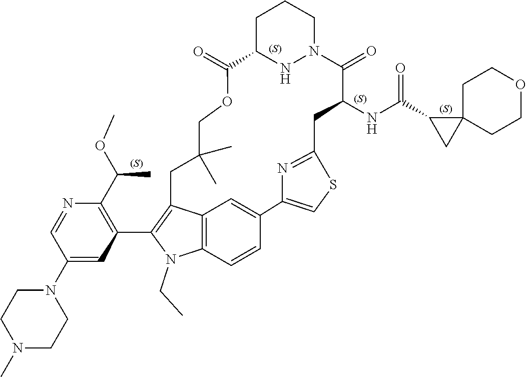 |
| DA99 | 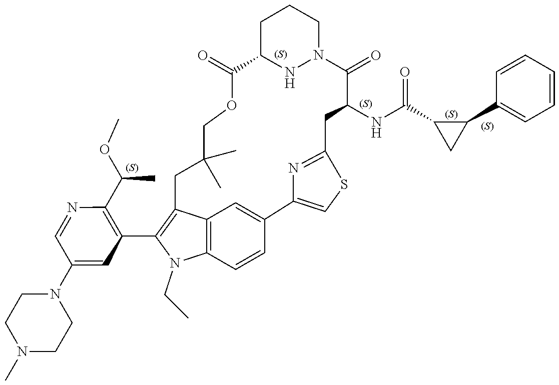 |
| DA100 | 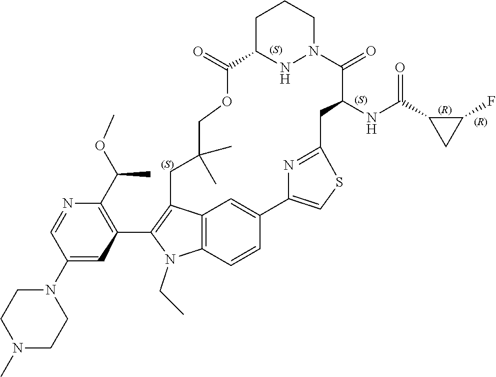 |
| DA101 | 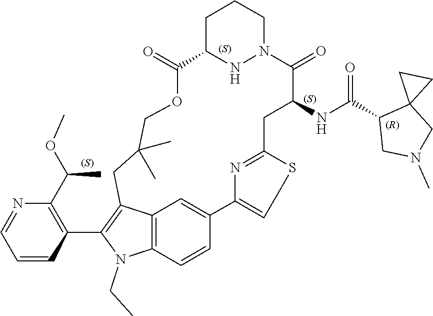 |
| DA102 | 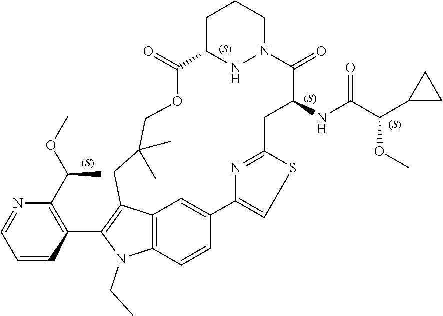 |
| DA103 | 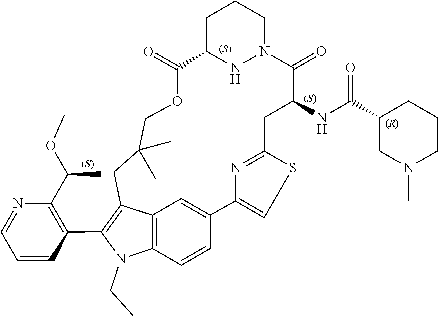 |
| DA104 | 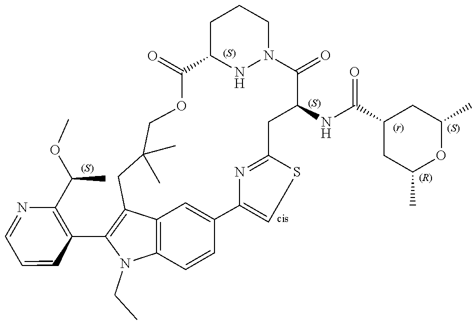 |
| DA105 | 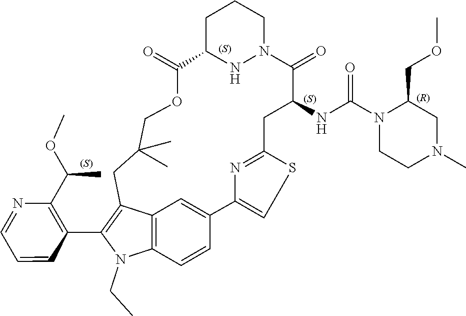 |
| DA106 | 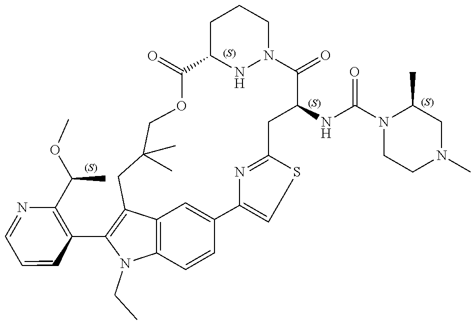 |
| DA107 | 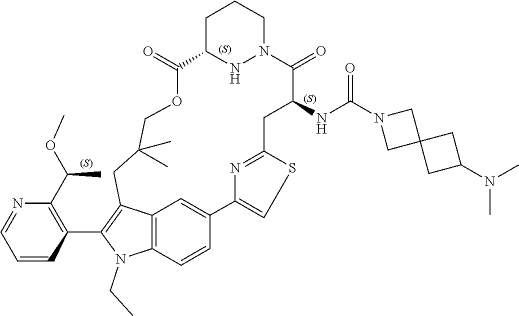 |
| DA108 | 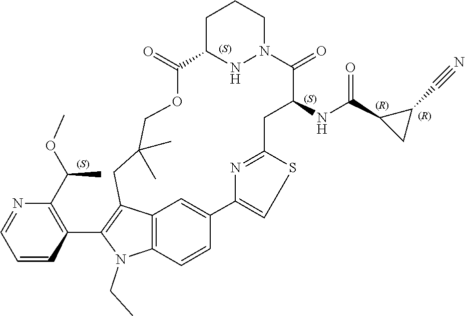 |
| DA109 | 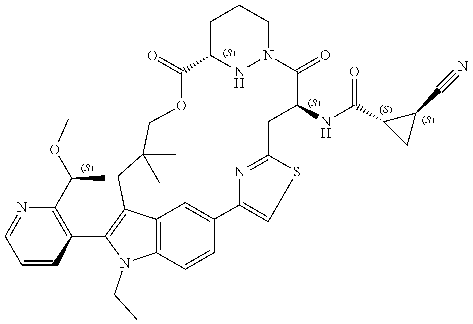 |
| DA110 | 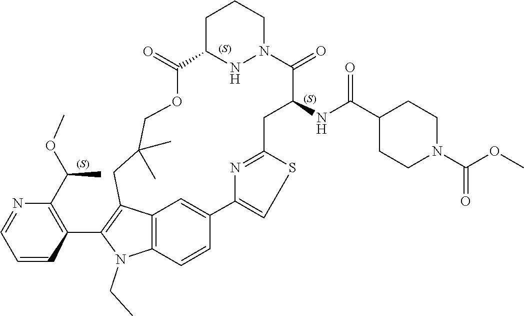 |
| DA111 | 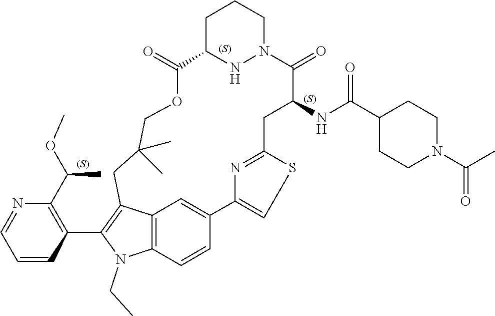 |
| DA112 | 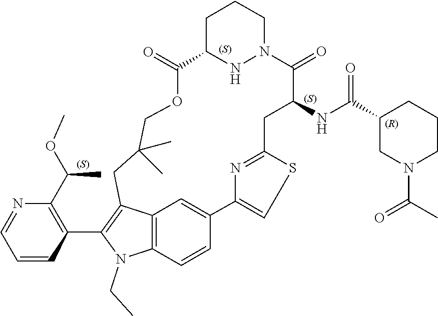 |
| DA113 | 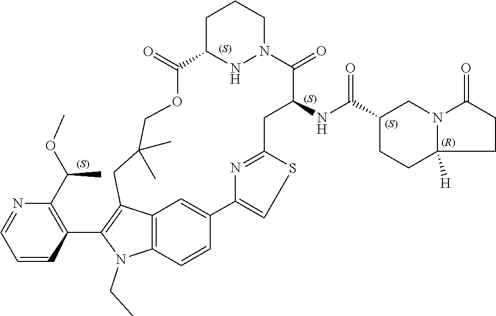 |
| DA114 | 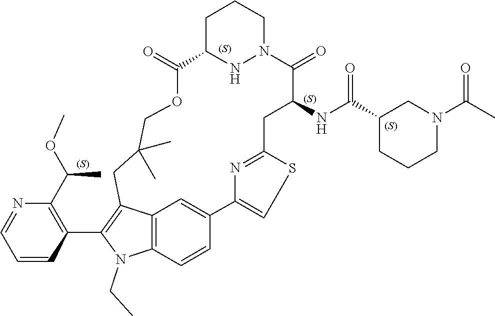 |
| DA115 | 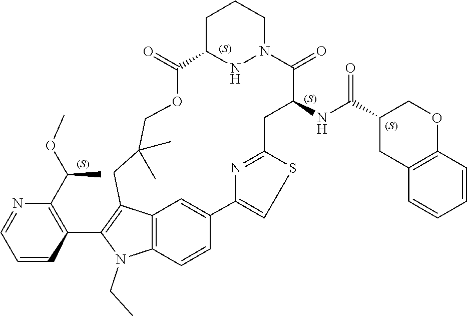 |
| DA116 | 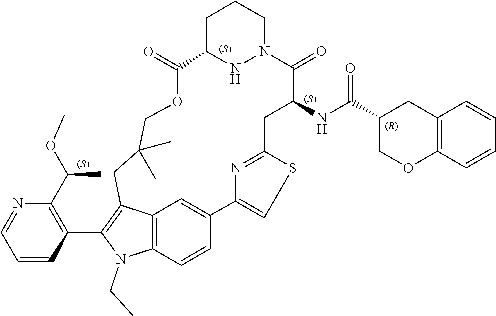 |
| DA117 | 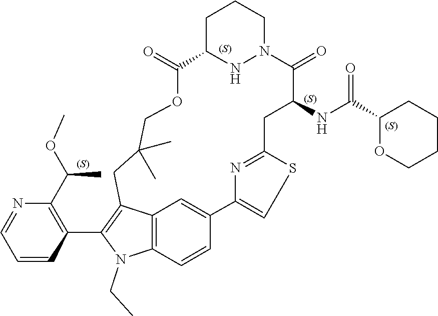 |
| DA118 | 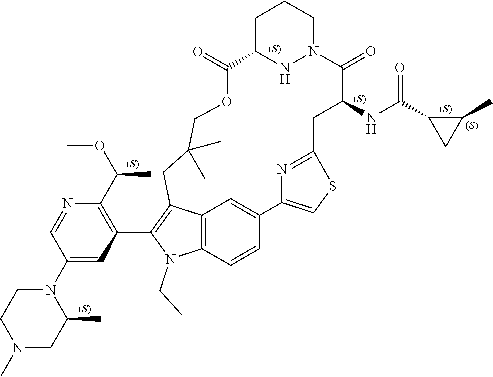 |
| DA119 | 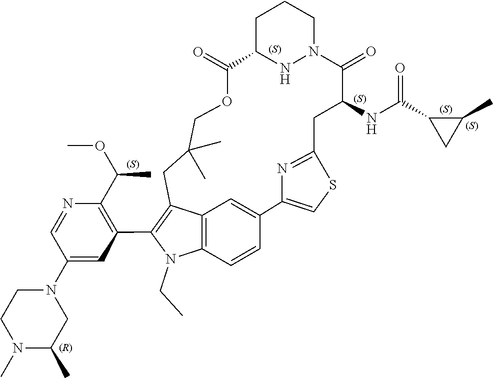 |
| DA120 | 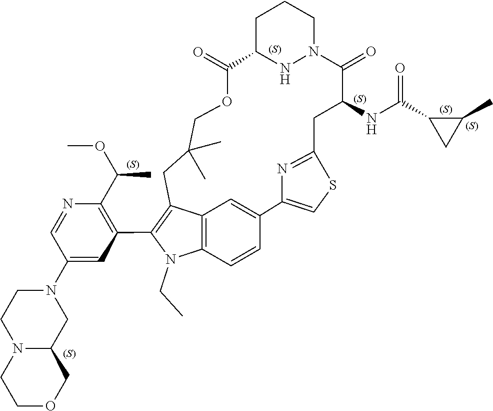 |
| DA121 | 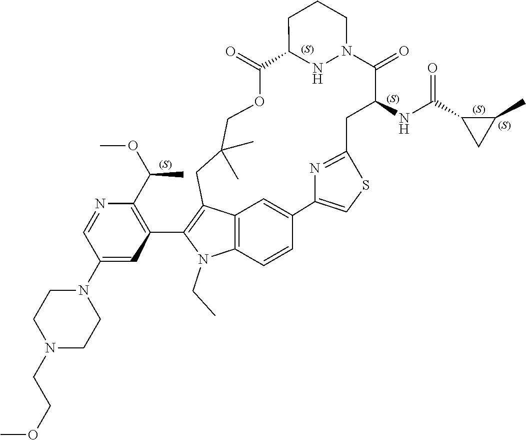 |
| DA122 | 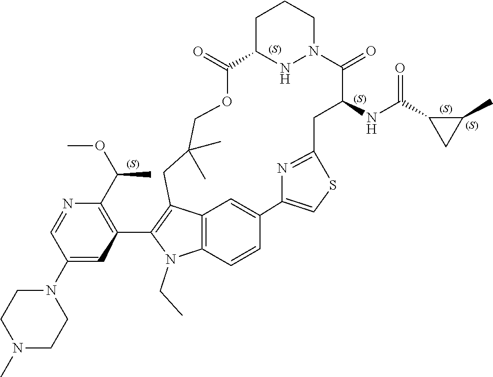 |
| DA123 | 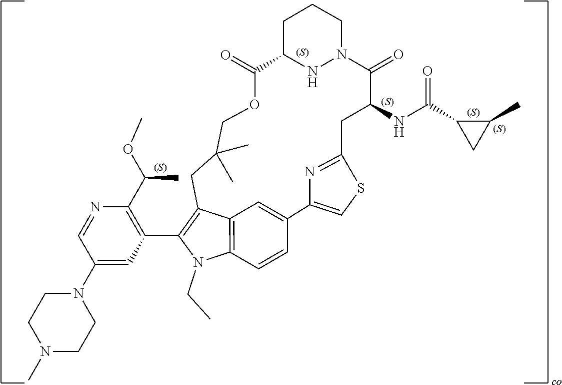 |
| DA124 | 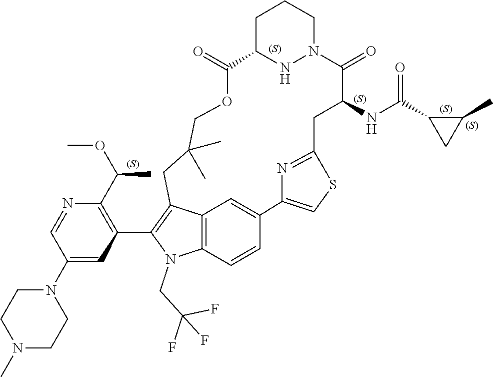 |
| DA125 | 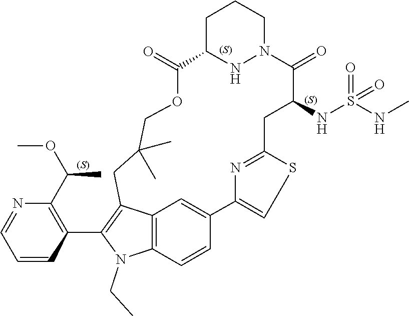 |
| DA126 | 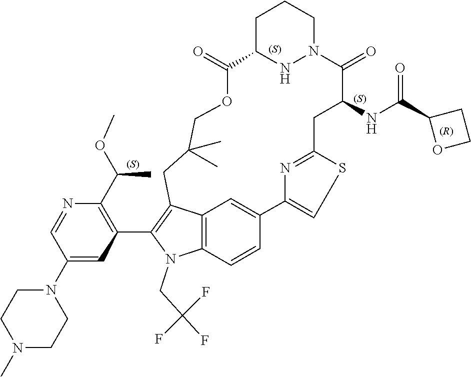 |
| DA127 | 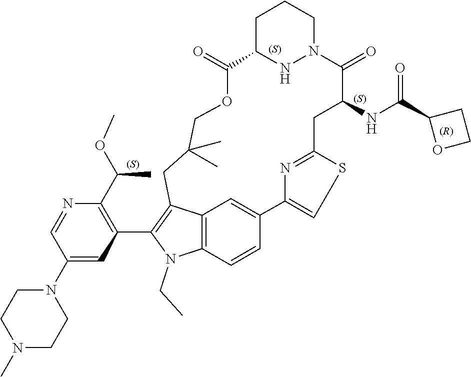 |
| DA128 | 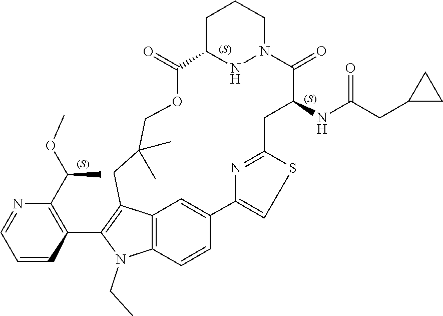 |
| DA129 | 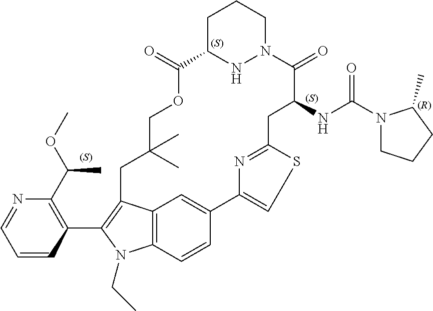 |
| DA130 | 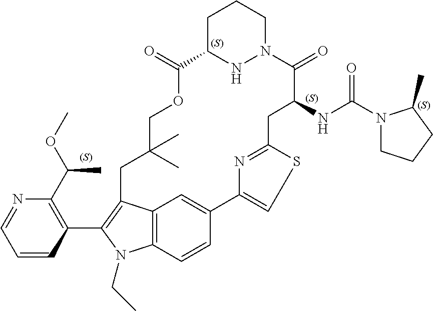 |
| DA131 | 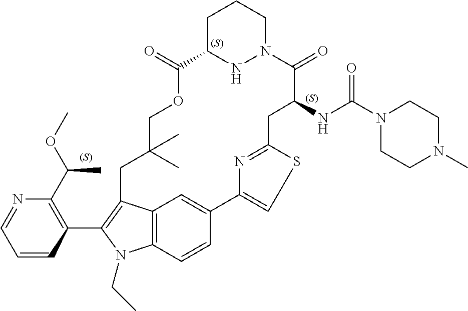 |
| DA132 | 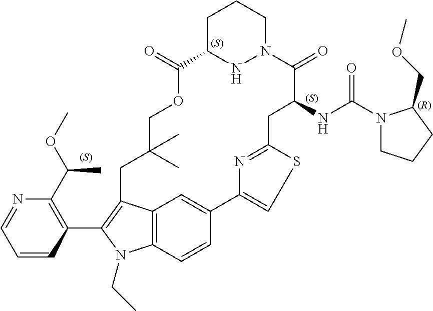 |
| DA133 | 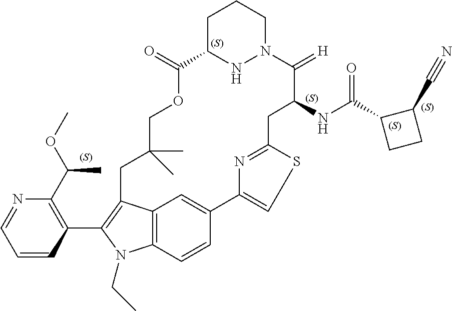 |
| DA133 | 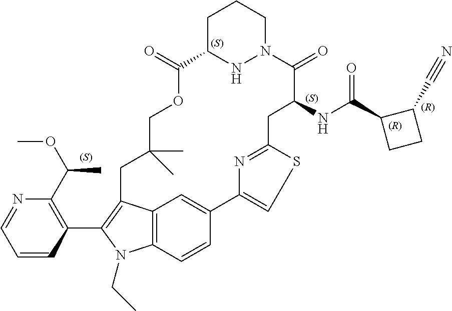 |
| DA134 | 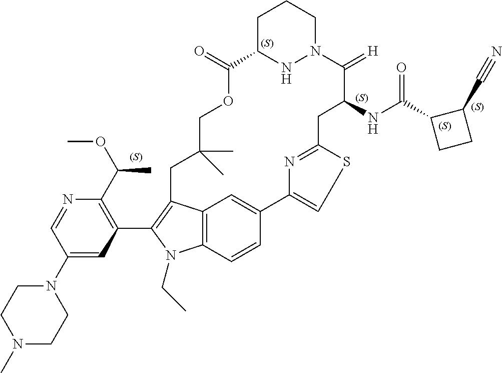 |
| DA135 | 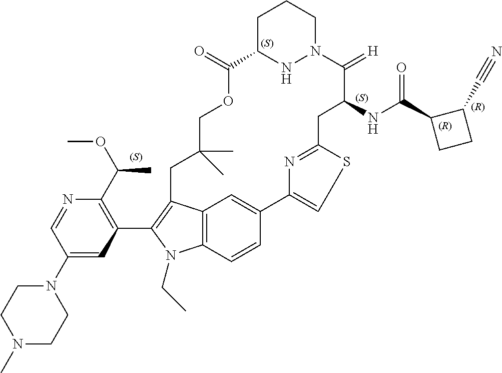 |
| DA136 | 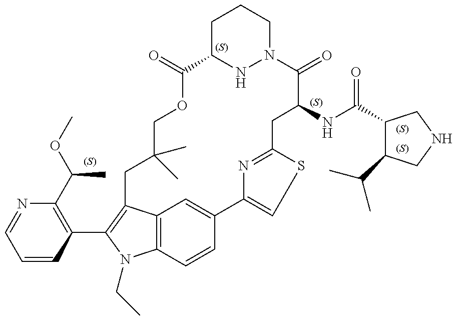 |
| DA137 | 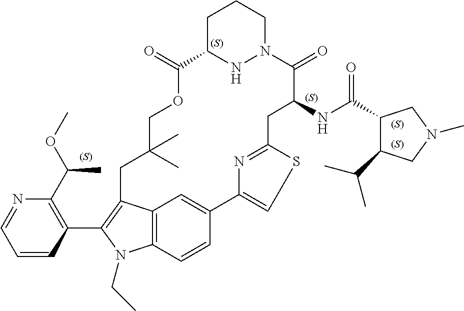 |
| DA138 | 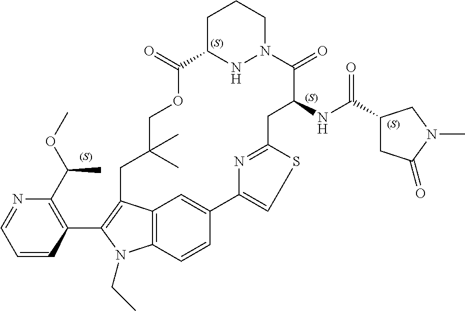 |
| DA139 | 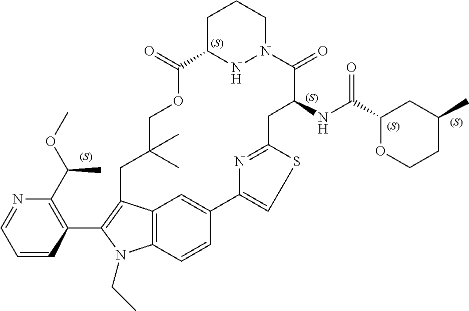 |
| DA140 | 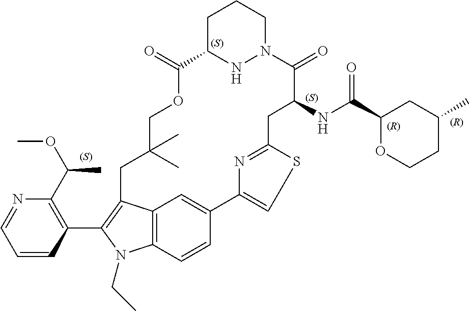 |
| DA141 | 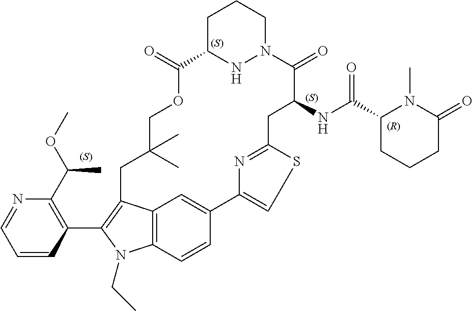 |
| DA142 | 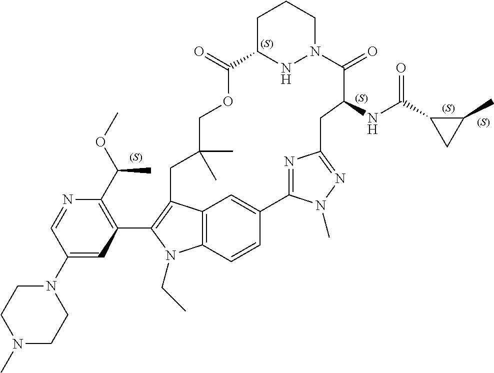 |
| DA143 | 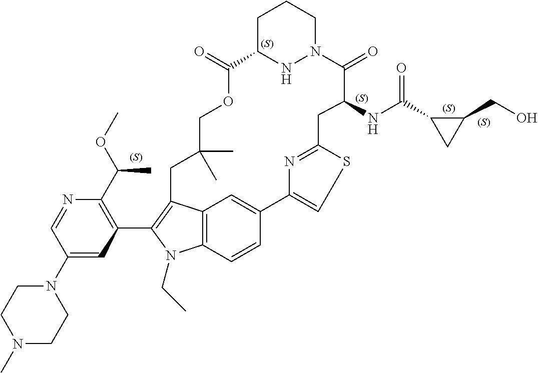 |
| DA144 | 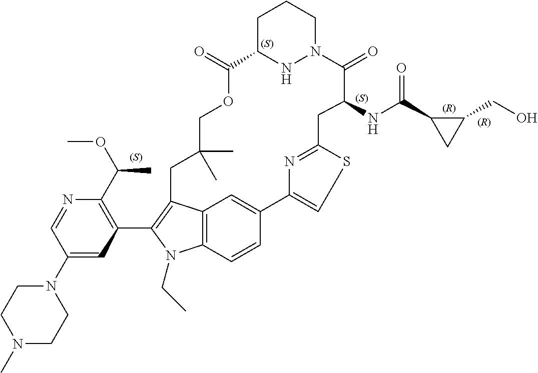 |
| DA145 | 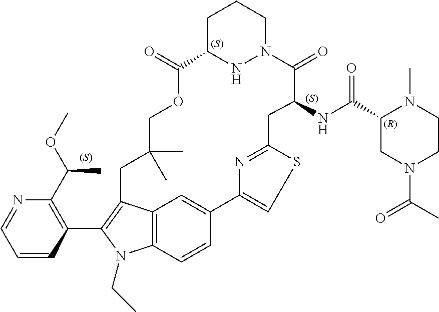 |
| DA146 | 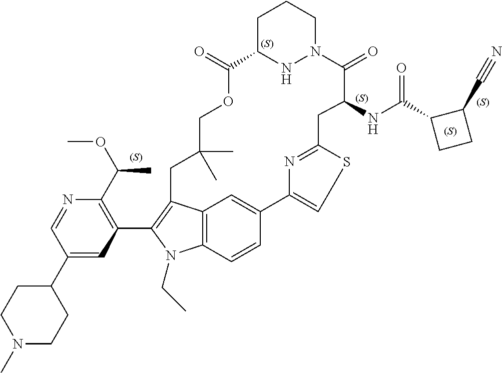 |
| DA147 | 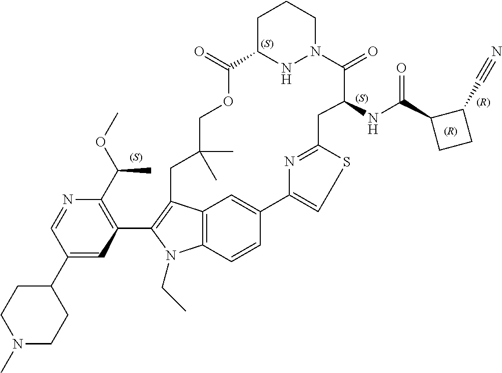 |
| DA148 | 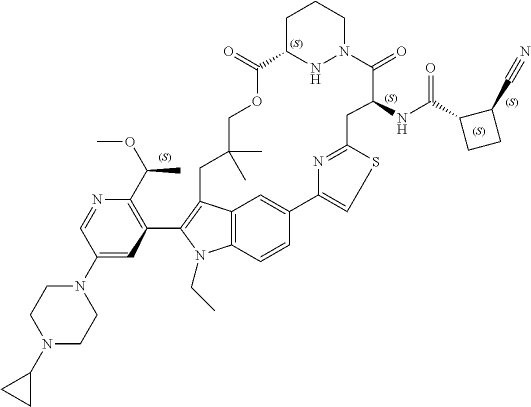 |
| DA149 | 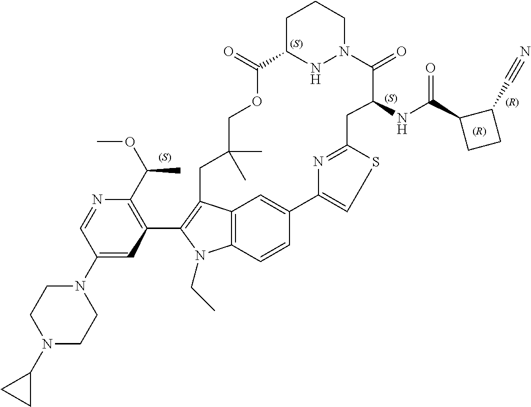 |
| DA150 | 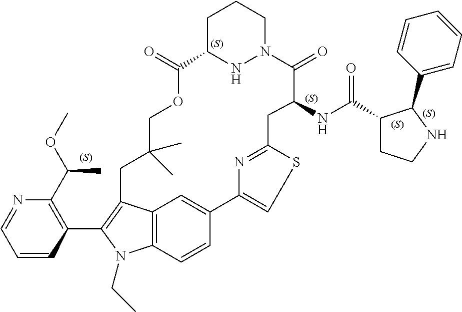 |
| DA151 | 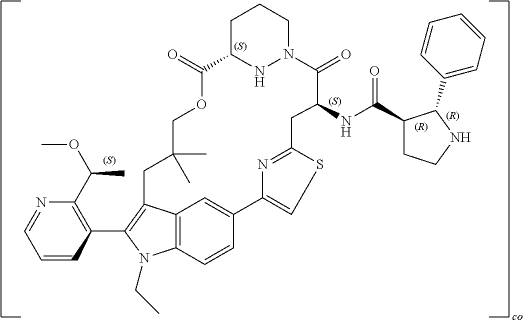 |
| DA152 | 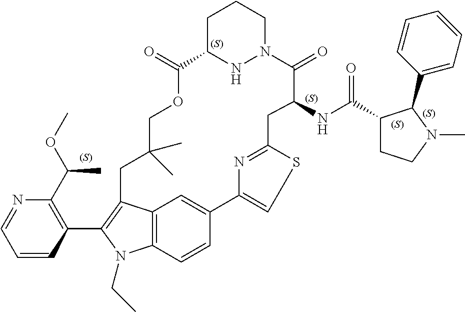 |
| DA153 | 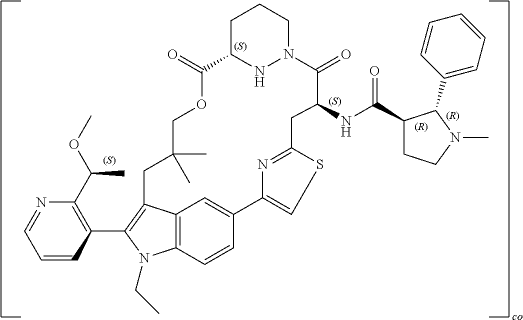 |
| DA154 | 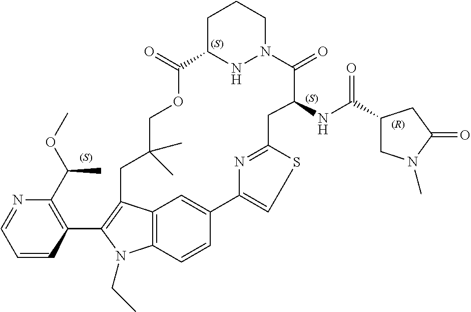 |
| DA155 | 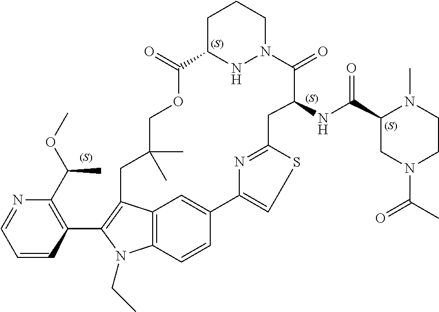 |
| DA156 | 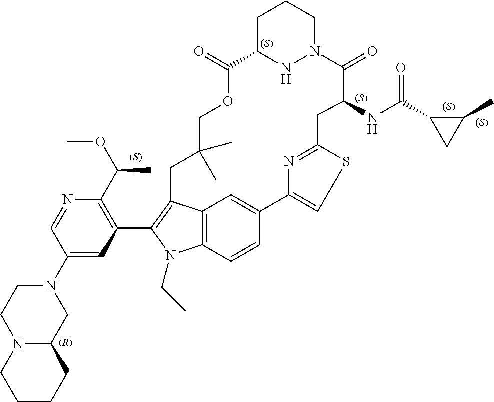 |
| DA157 | 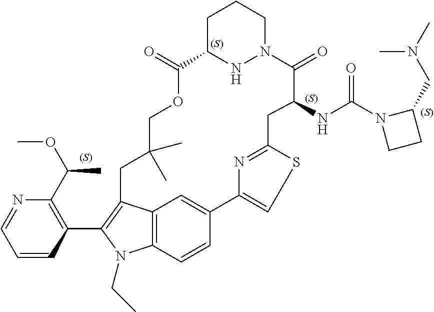 |
| DA158 | 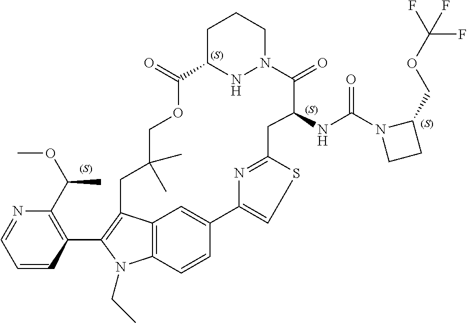 |
| DA159 | 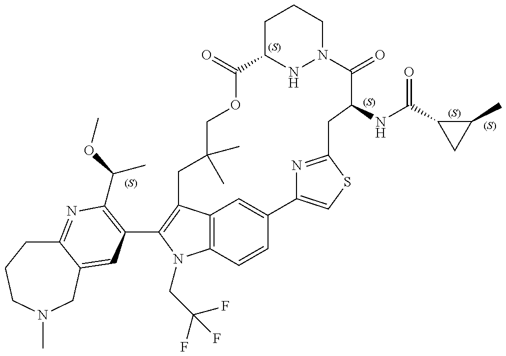 |
| DA160 | 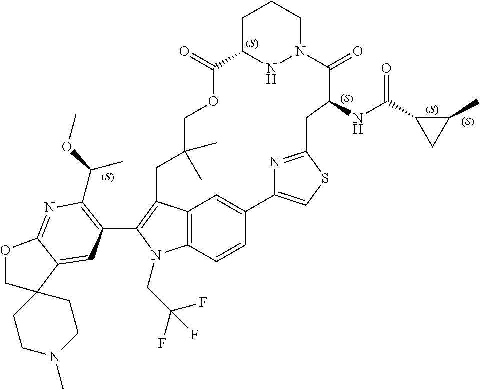 |
| DA161 | 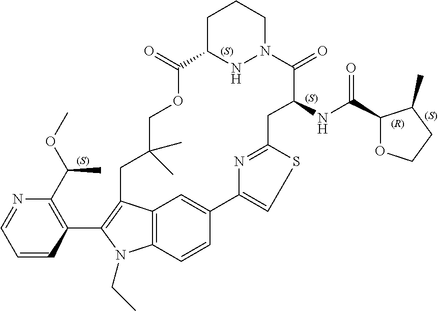 |
| DA162 | 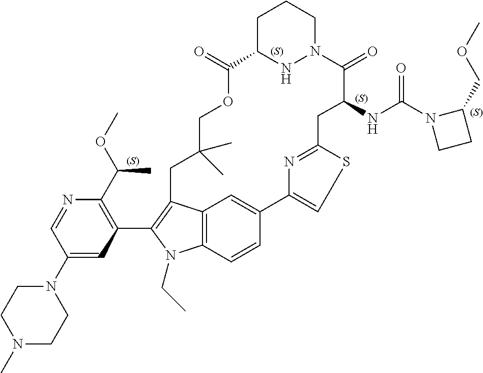 |
| DA163 | 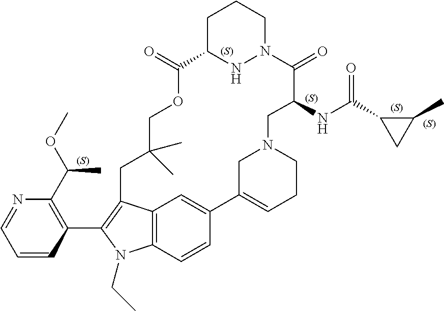 |
| DA164 | 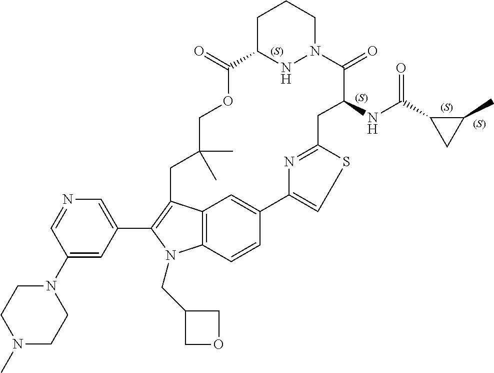 |
| DA165 | 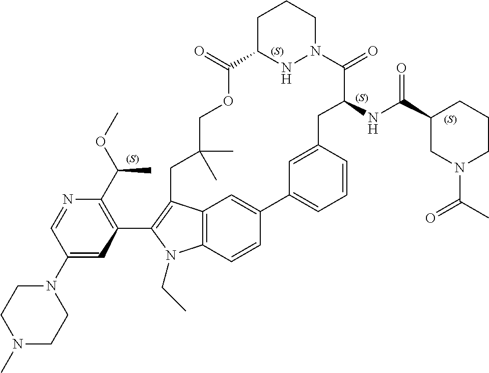 |
| DA166 | 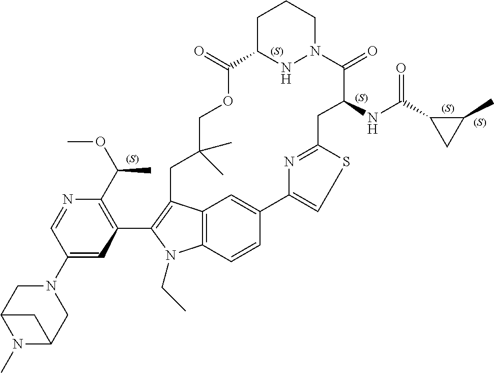 |
| DA167 | 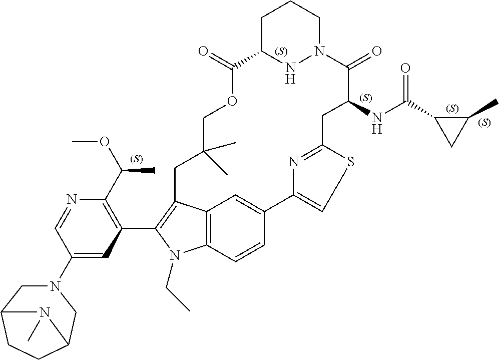 |
| DA168 | 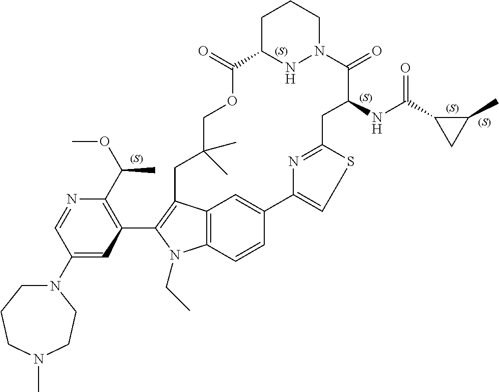 |
| DA169 | 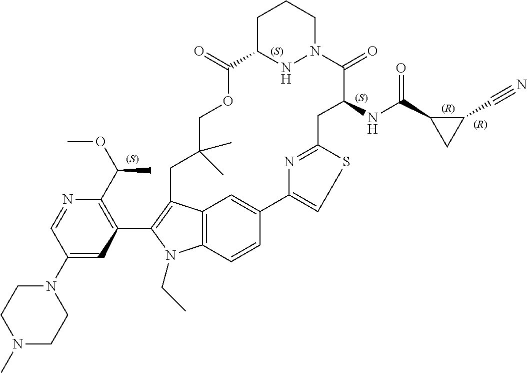 |
| DA170 | 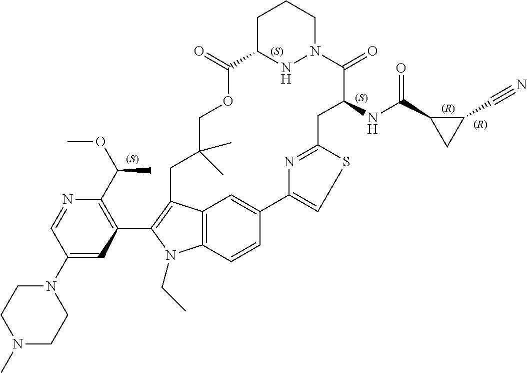 |
| DA171 | 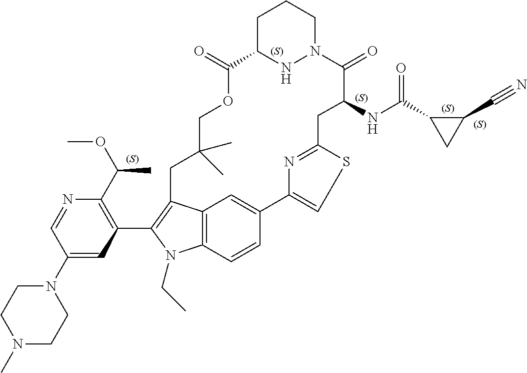 |
| DA172 | 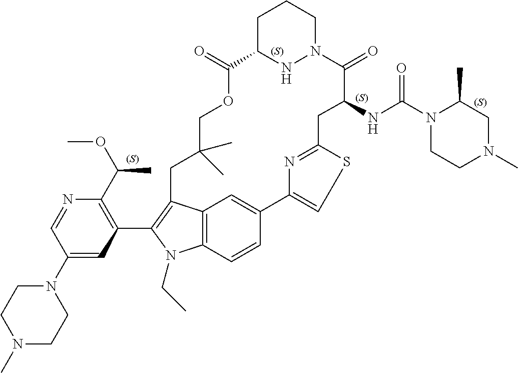 |
| DA173 | 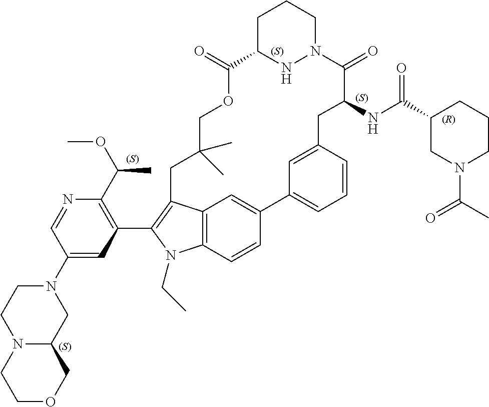 |
| DA174 | 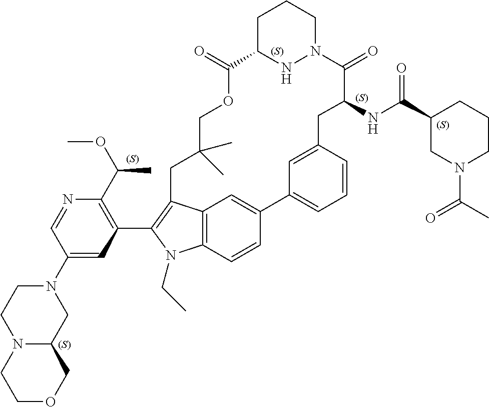 |
| DA175 | 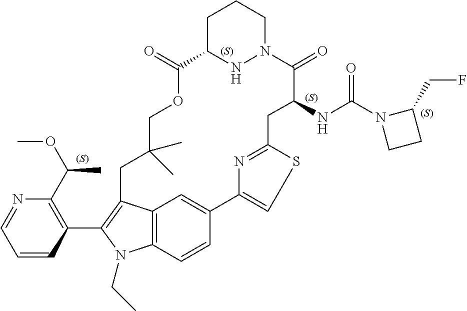 |
| DA176 | 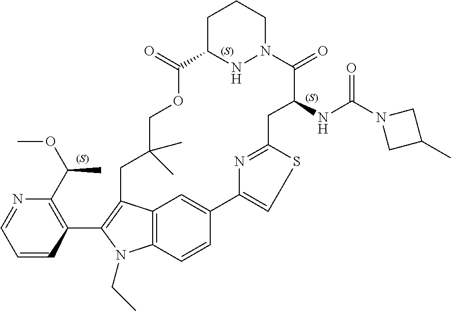 |
| DA177 | 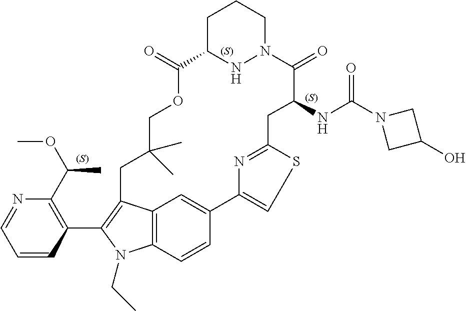 |
| DA178 | 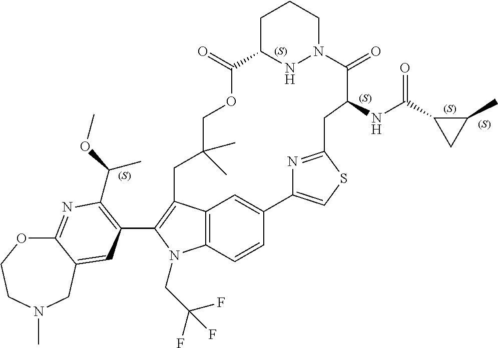 |
| DA179 | 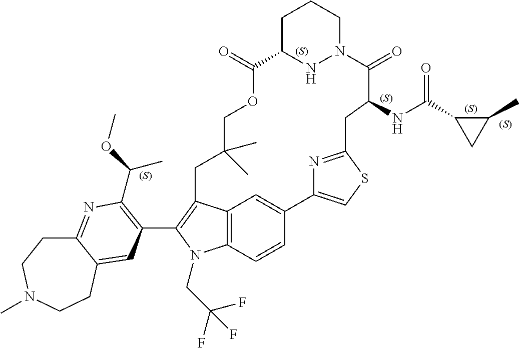 |
| DA180 | 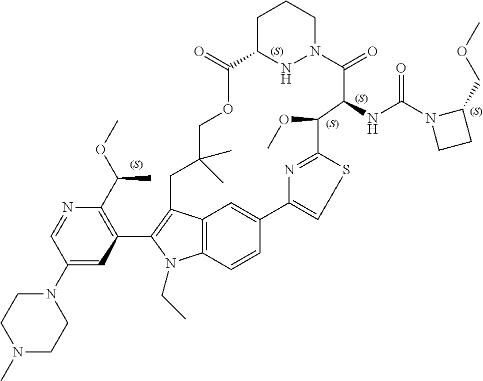 |
| DA181 | 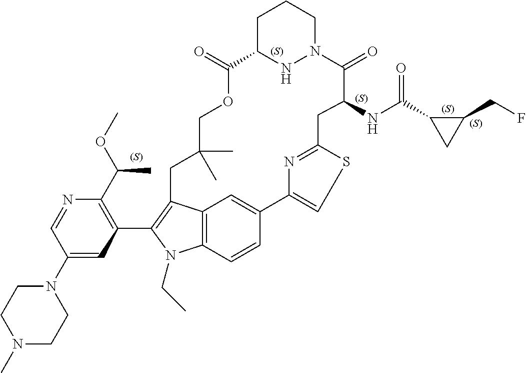 |
| DA182 | 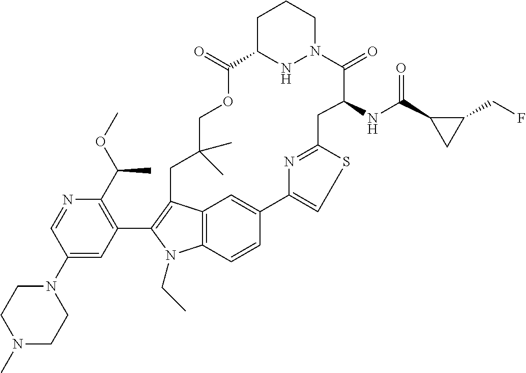 [SMILES: CCn1c(-c2cc(N3CCN(C)CC3)cnc2[C@H](C)OC)c2c3cc(ccc31)-c1csc(n1)C[C@H](NC(=O)[C@@H]1C[C@H]1CF)C(=O)N1CCC[C@H](N1)C(=O)OCC(C)(C)C2] |
| DA183 | 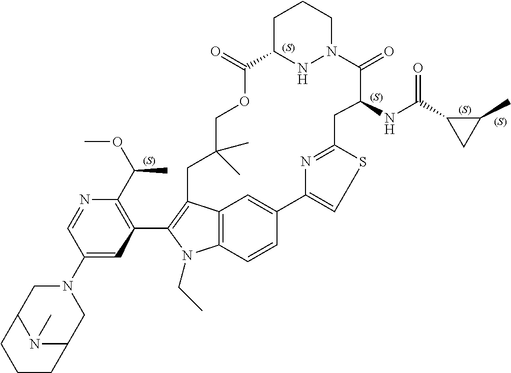 |
| DA184 | 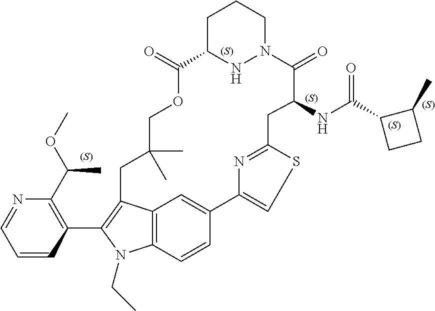 |
| DA185 | 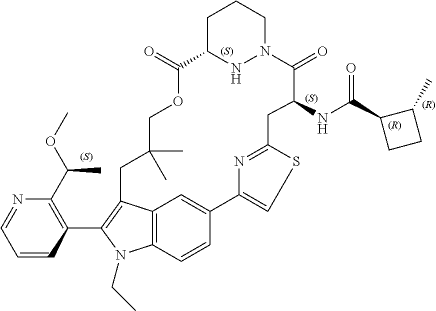 |
| DA186 | 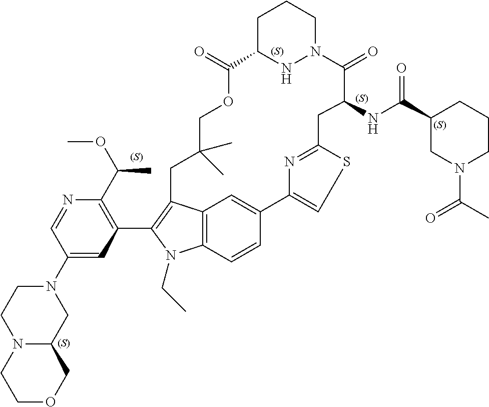 |
| DA187 | 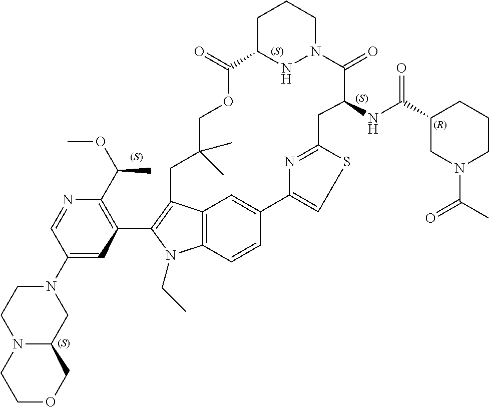 |
| DA188 | 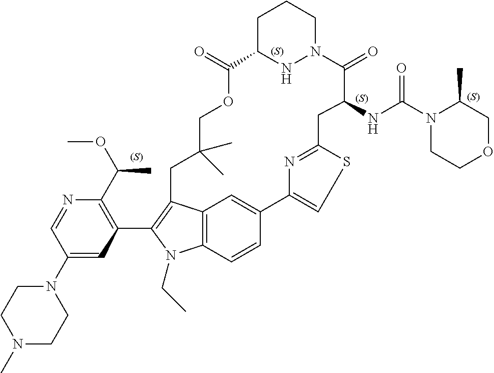 |
| DA189 | 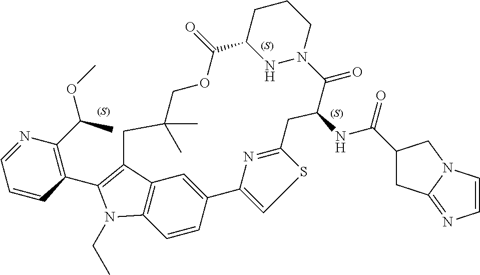 |
| DA190 | 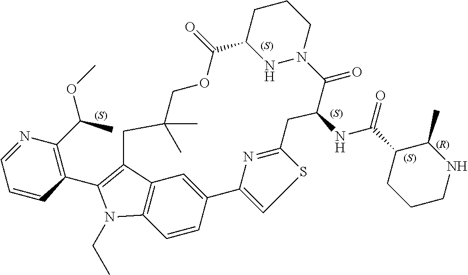 |
| DA191 | 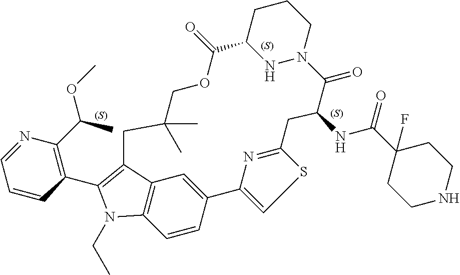 |
| DA192 | 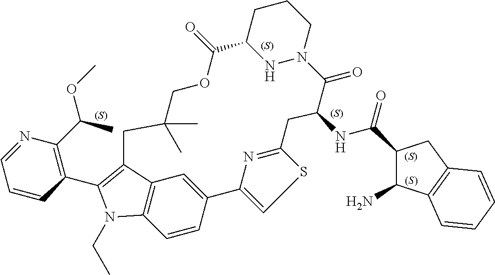 |
| DA193 | 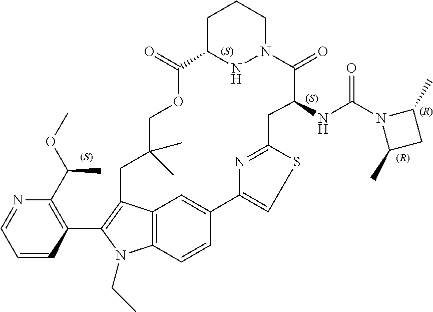 |
| DA194 | 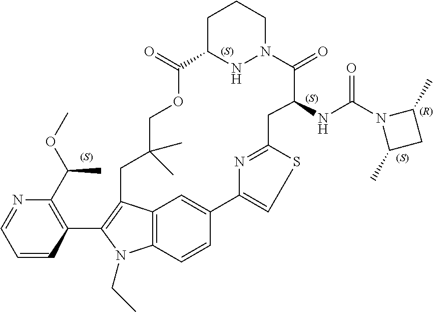 |
| DA195 | 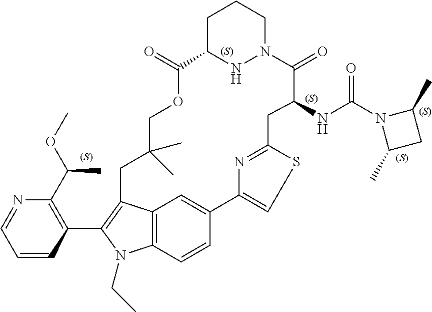 |
| DA196 | 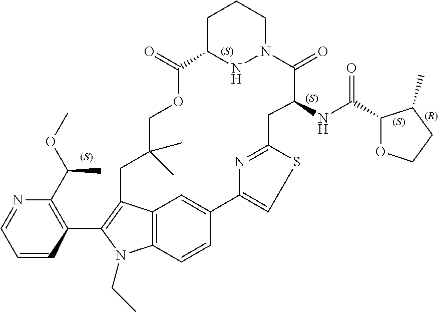 |
| DA197 | 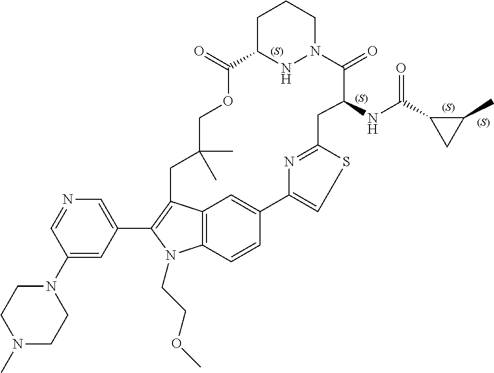 |
| DA198 | 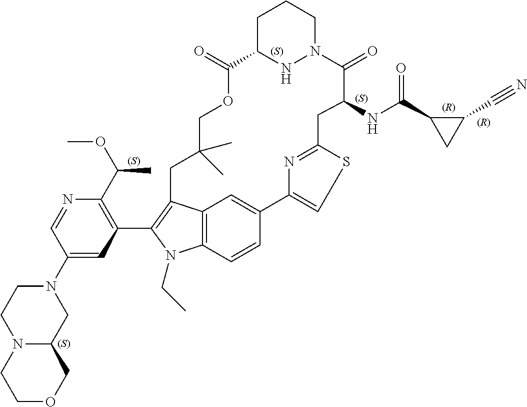 |
| DA199 | 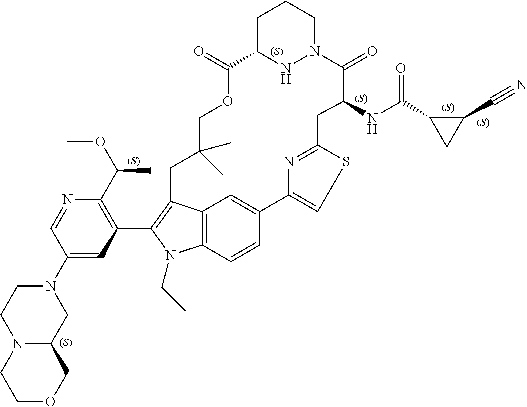 |
| DA200 | 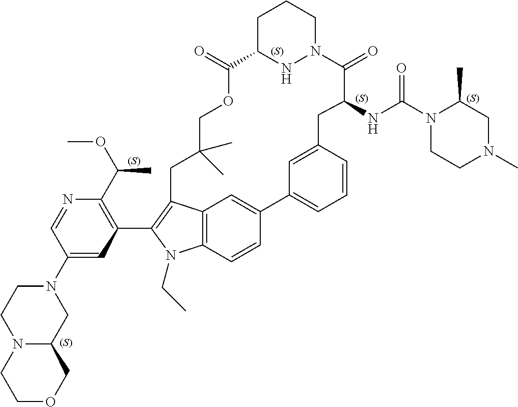 |
| DA201 | 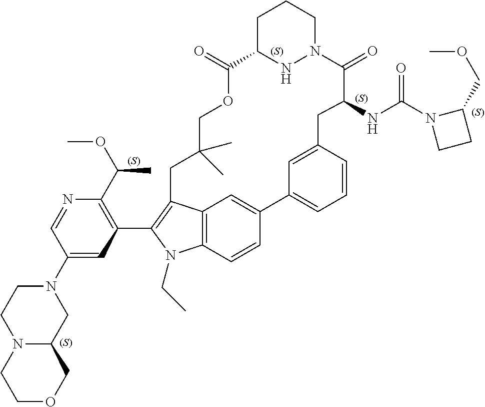 |
| DA202 | 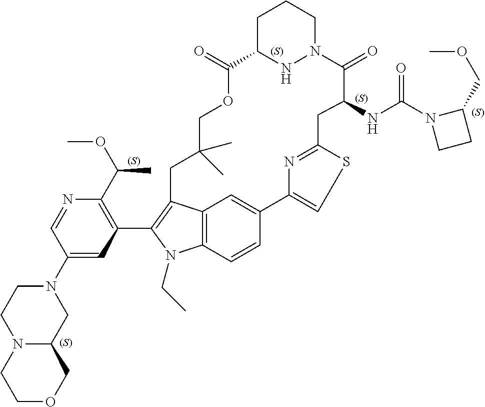 |
| DA203 | 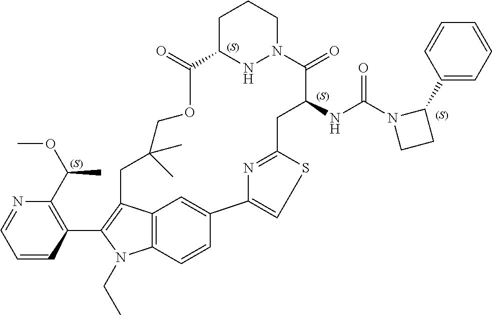 |
| DA204 | 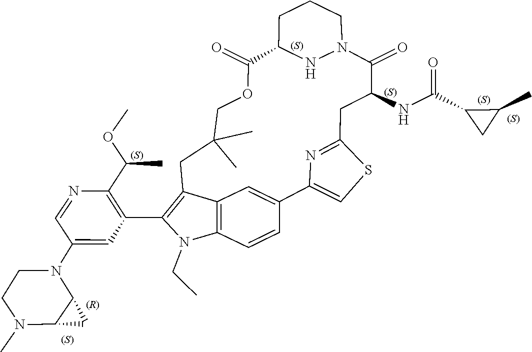 |
| DA205 | 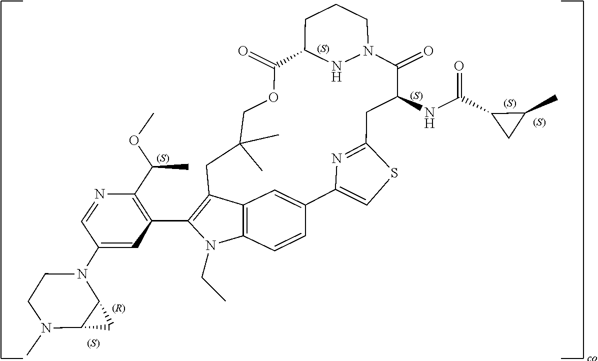 |
| DA206 | 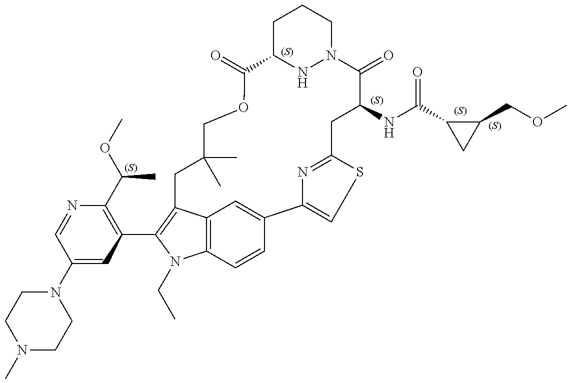 |
| DA207 | 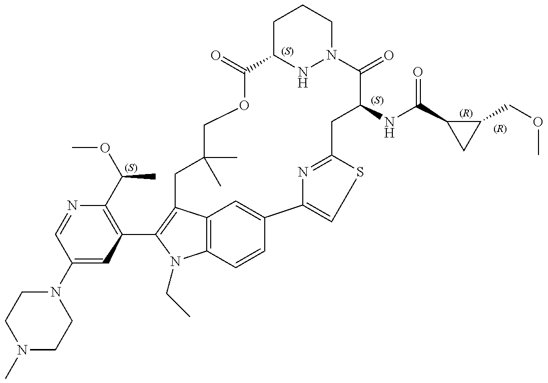 |
| DA208 | 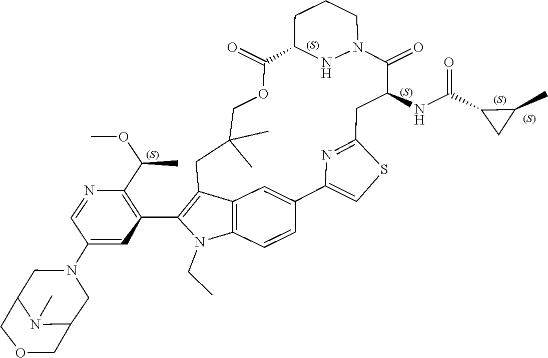 |
| DA209 | 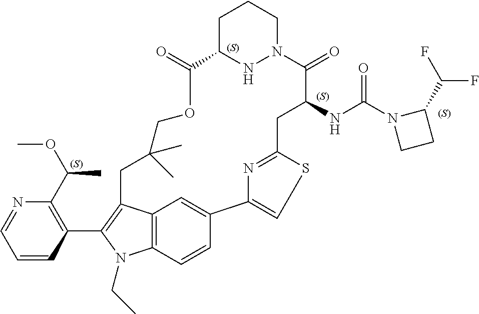 |
| DA210 | 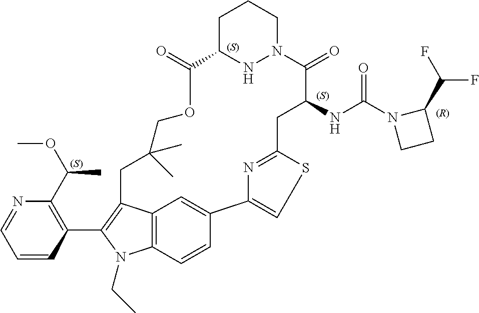 |
| DA211 | 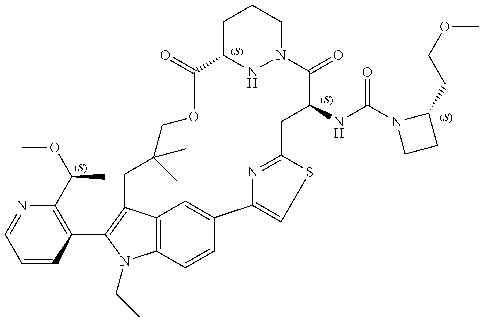 |
| DA212 | 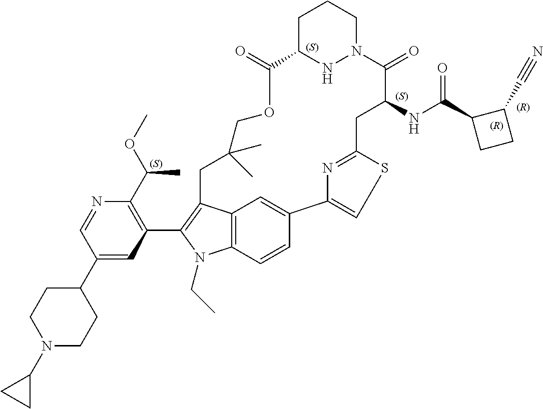 |
| DA213 | 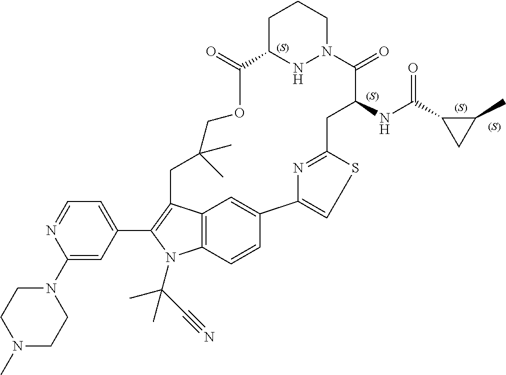 |
| DA214 | 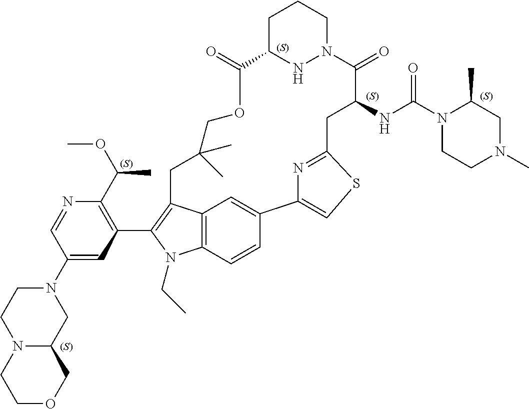 |
| DA215 | 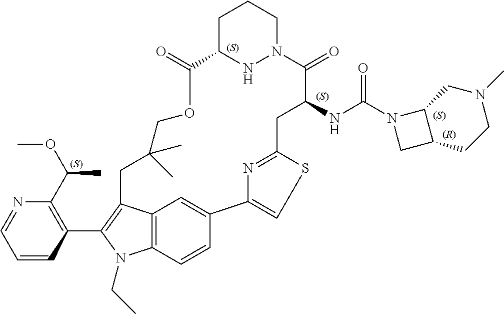 |
| DA216 | 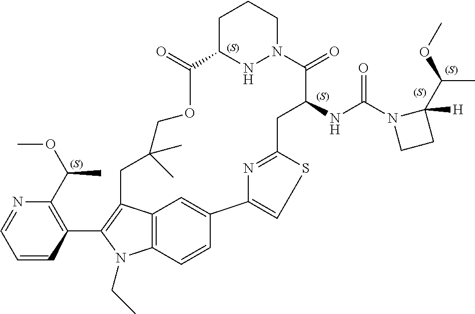 |
| DA217 | 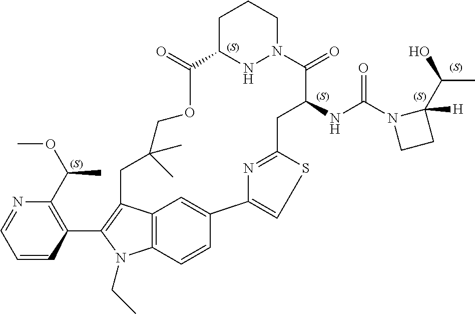 |
| DA218 | 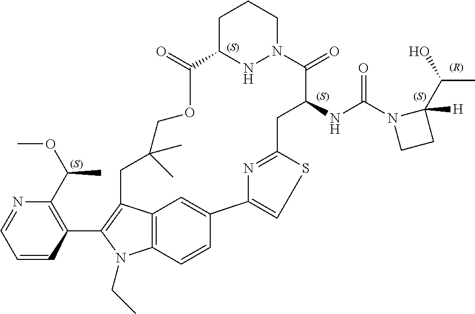 |
| DA219 | 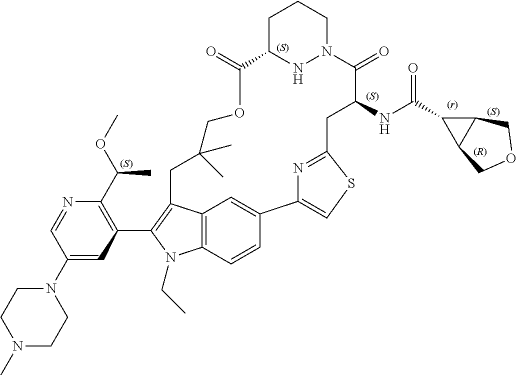 |
| DA220 | 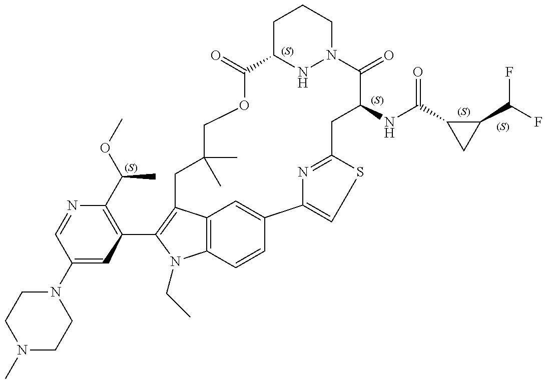 |
| DA221 | 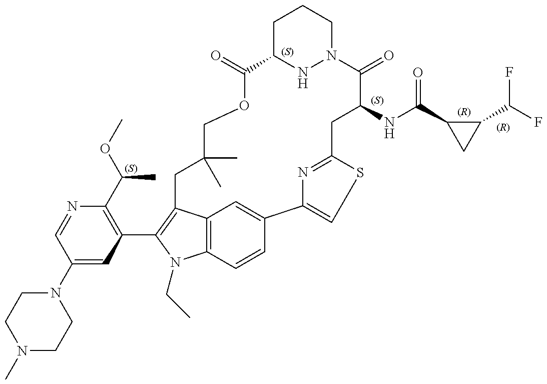 |
| DA222 | 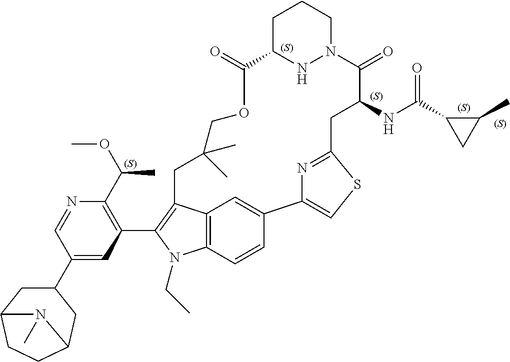 |
| DA223 | 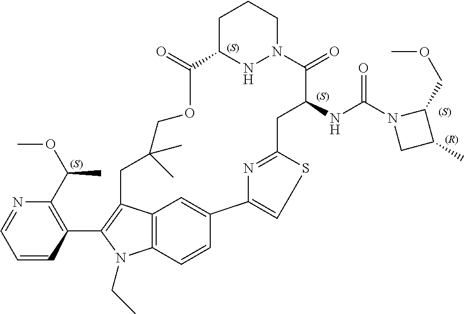 |
| DA224 | 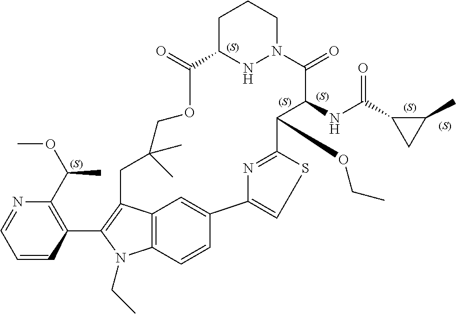 |
| DA225 | 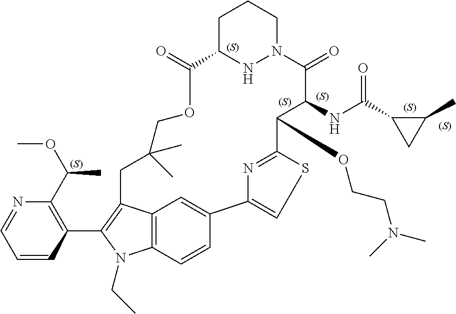 |
| DA226 | 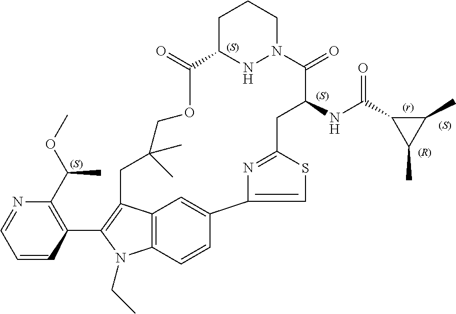 |
| DA227 | 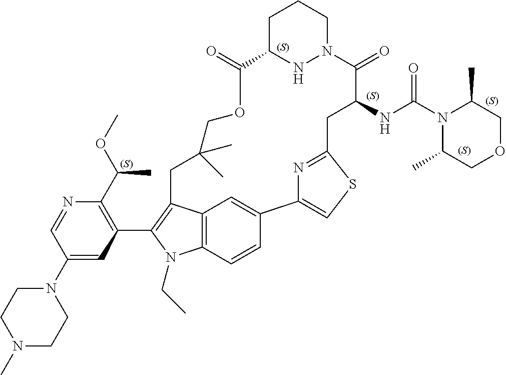 |
| DA228 | 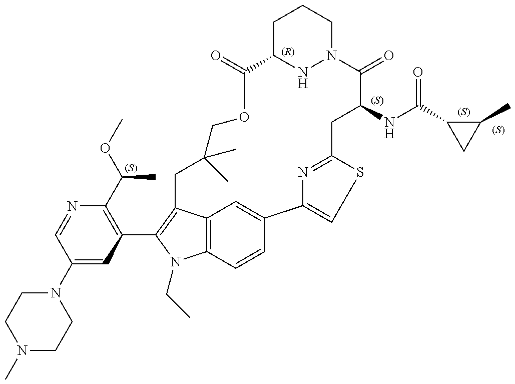 |
| DA229 | 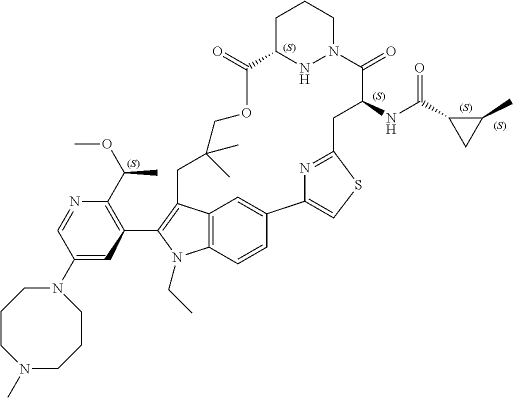 |
| DA230 | 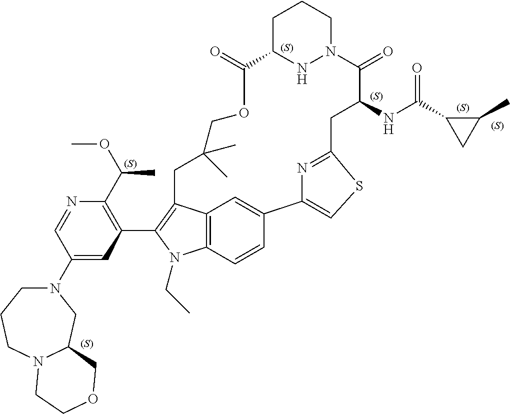 |
| DA231 | 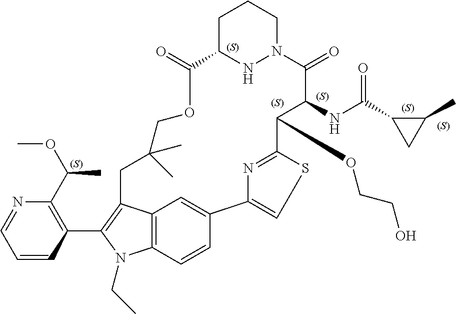 |
| DA232 | 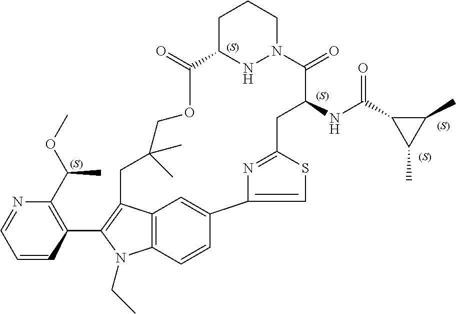 |
| DA233 | 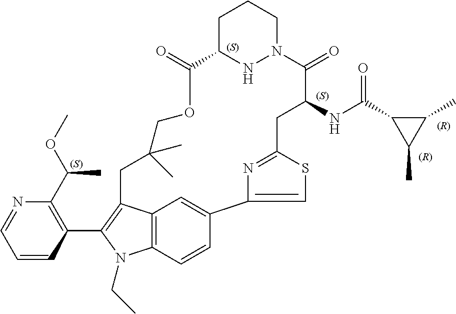 |
| DA234 | 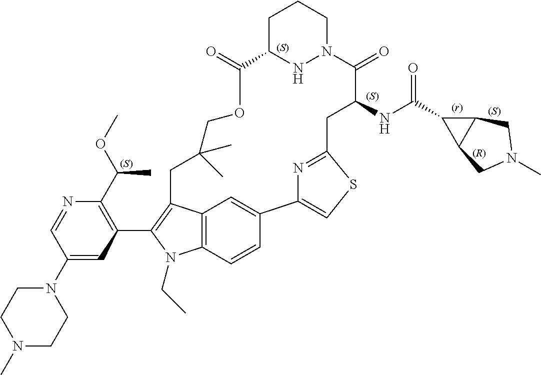 |
| DA235 | 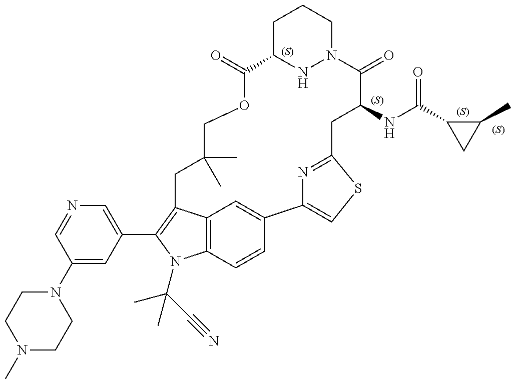 |
| DA236 | 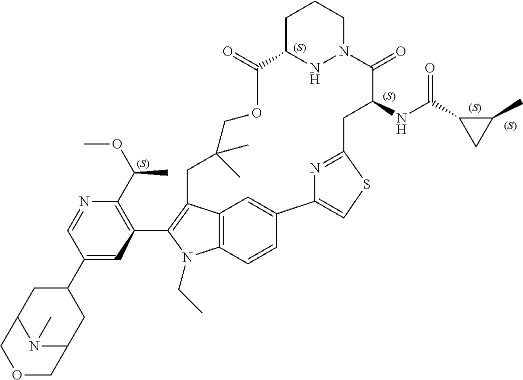 |
| DA237 | 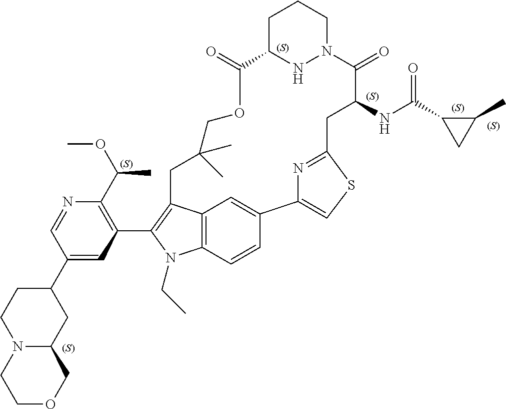 |
| DA238 | 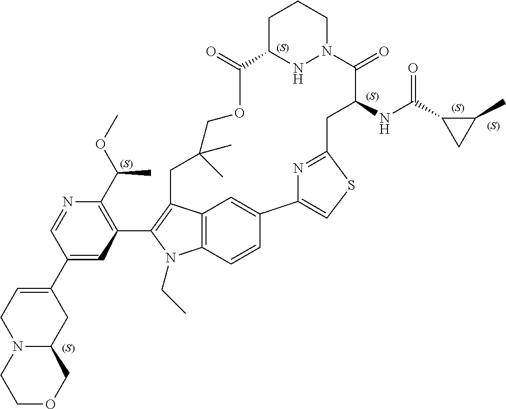 |
| DA239 | 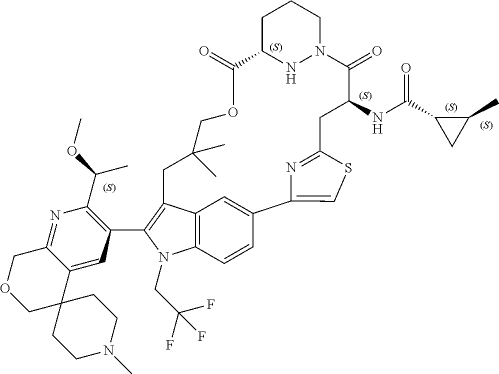 |
| DA240 | 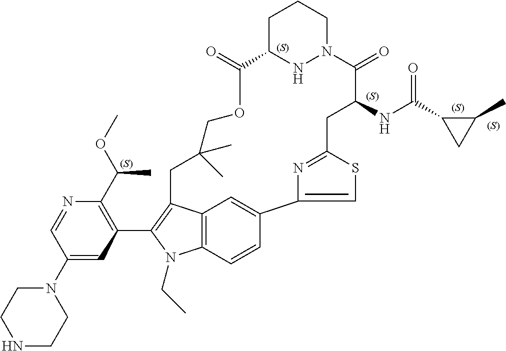 |
| DA241 | 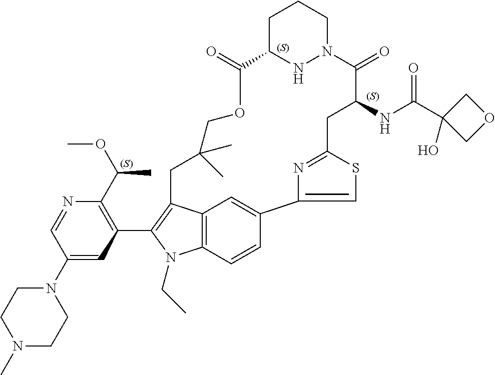 |
| DA242 | 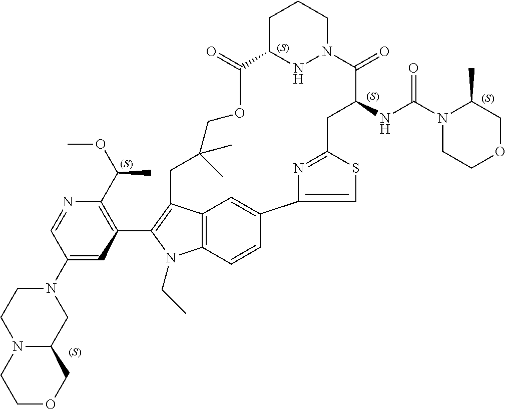 |
| DA243 | 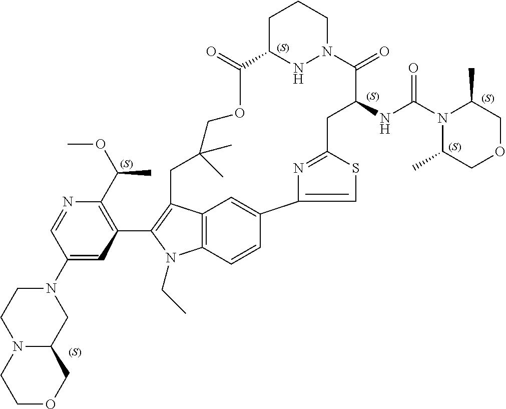 |
| DA244 | 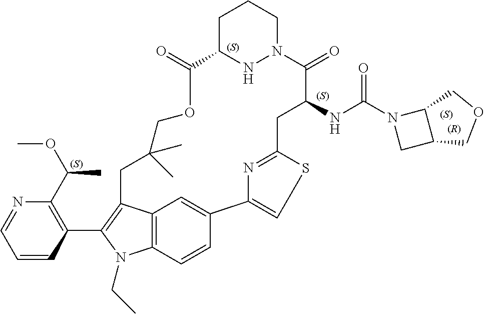 |
| DA245 | 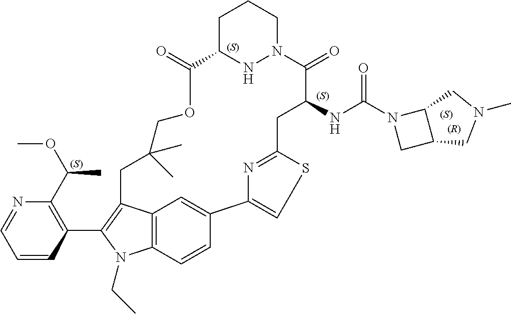 |
| DA246 | 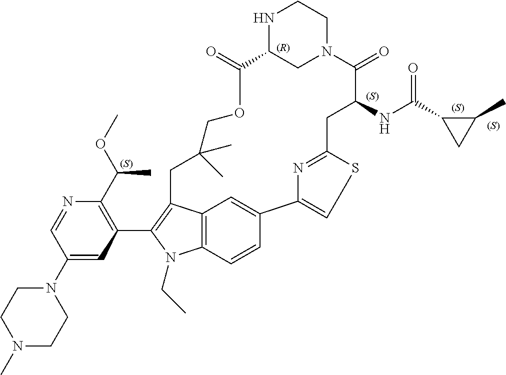 |
| DA247 | 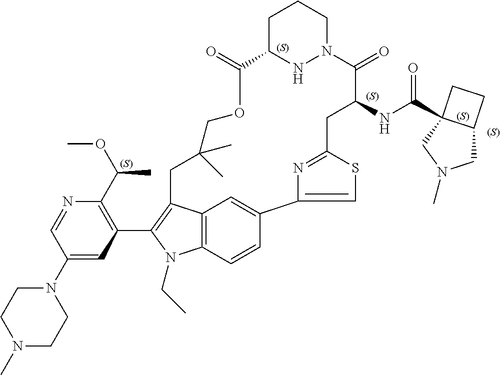 |
| DA248 | 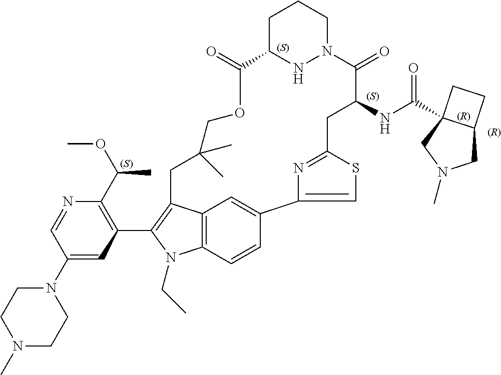 |
| DA249 | 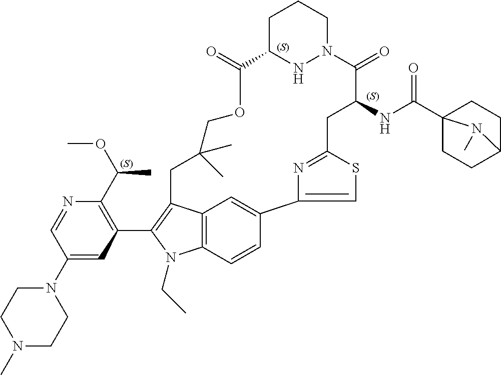 |
| DA250 | 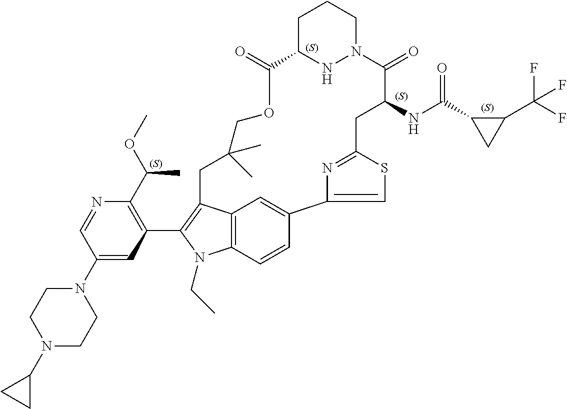 |
| DA251 | 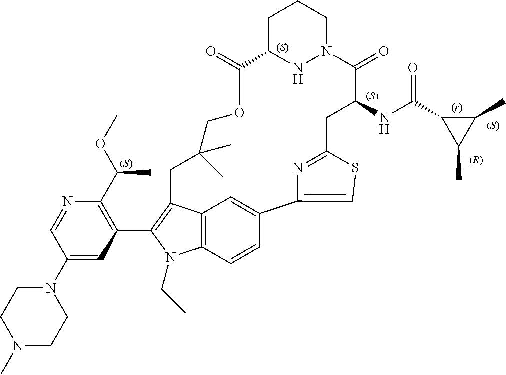 |
| DA252 | 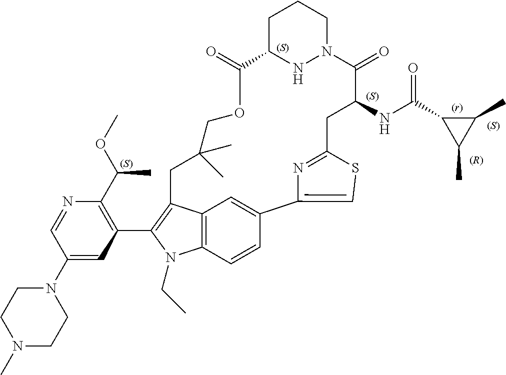 |
| DA253 | 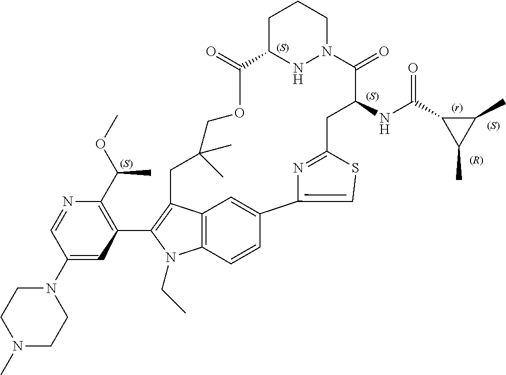 |
| DA254 | 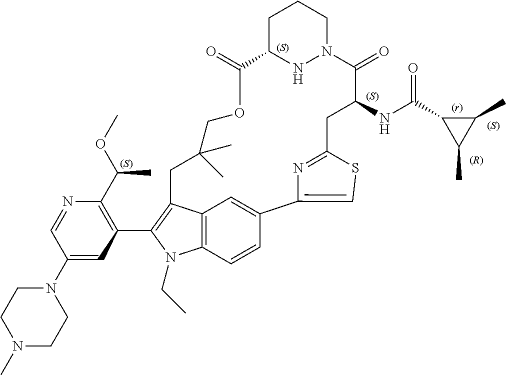 |
| DA255 | 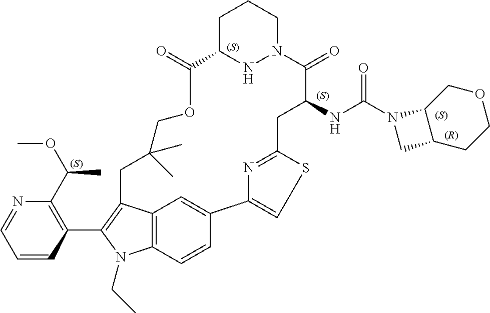 |
| DA256 | 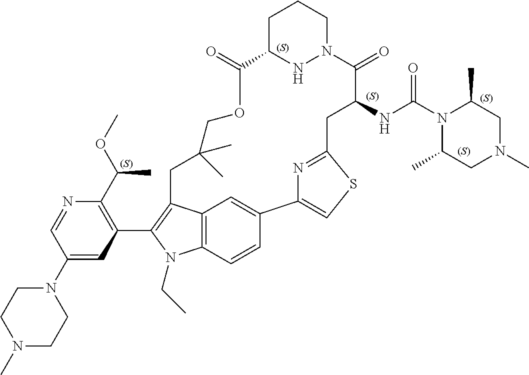 |
| DA257 | 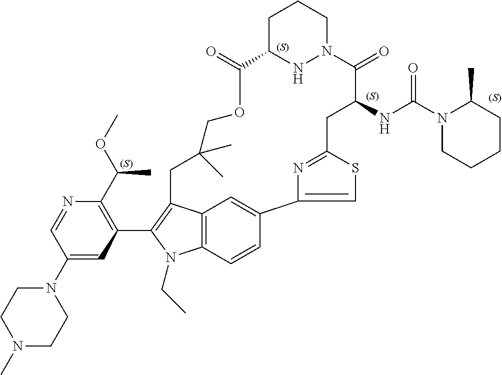 |
| DA258 | 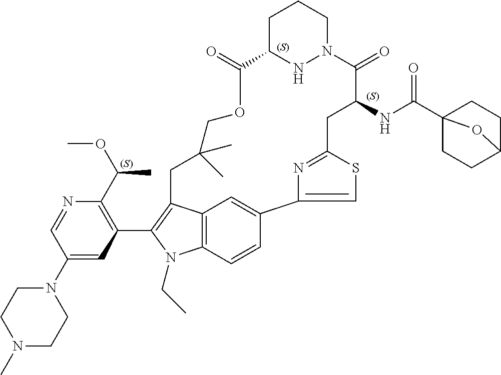 |
| DA259 | 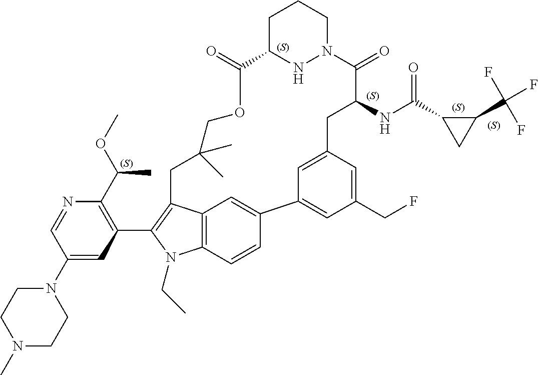 |
| DA260 | 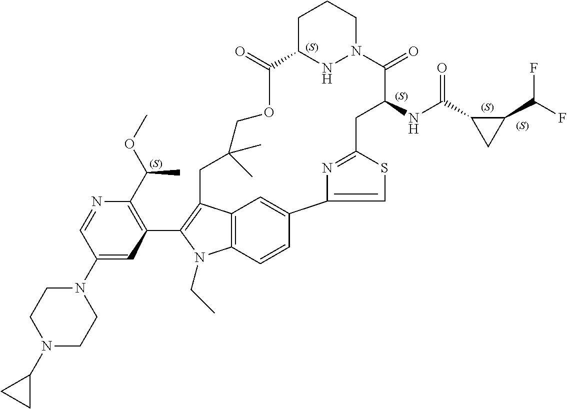 |
| DA261 | 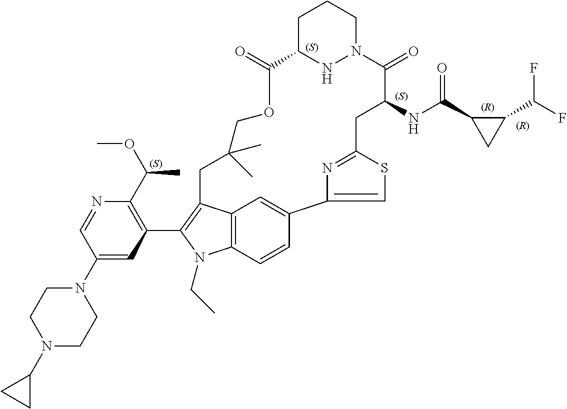 |
| DA262 | 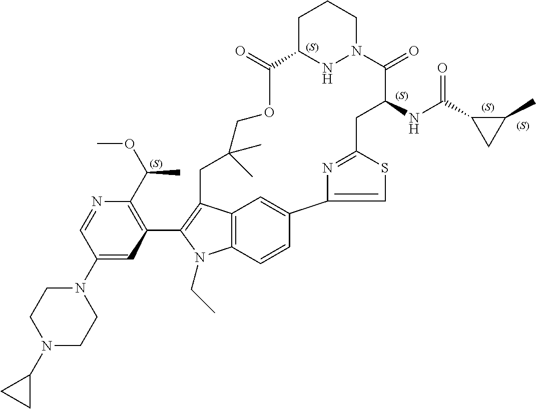 |
| DA263 | 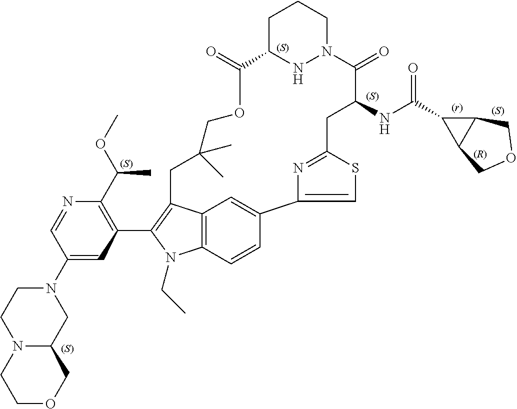 |
| DA264 | 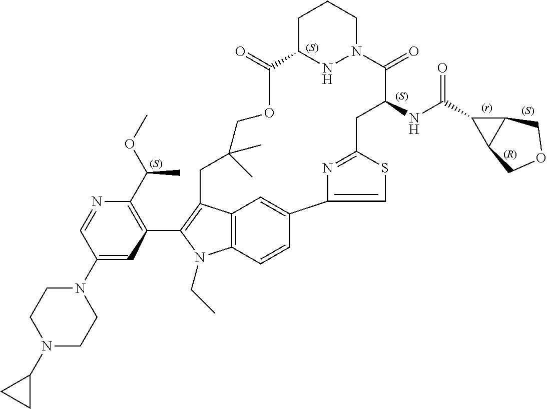 |
| DA265 | 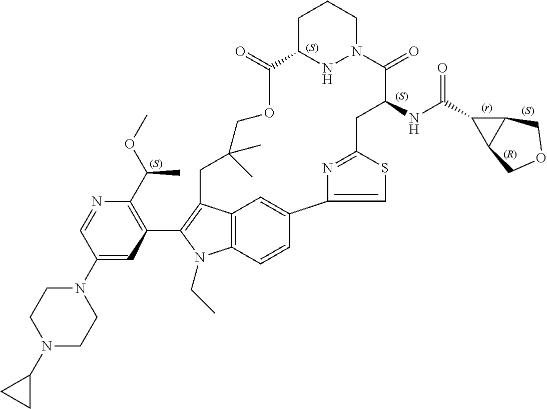 |
| DA266 | 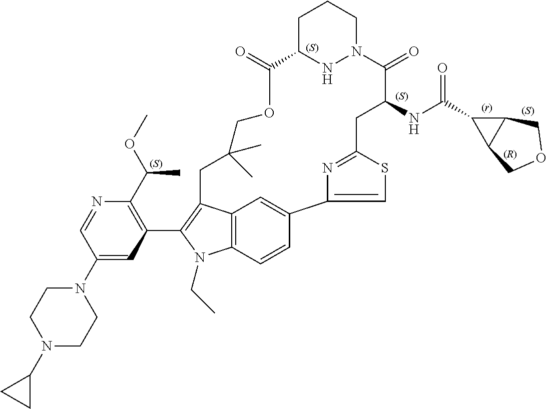 |
| DA267 | 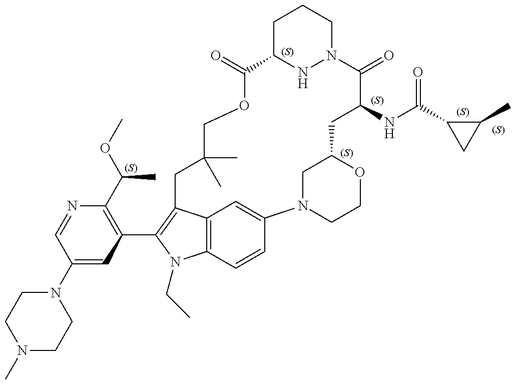 |
| DA268 | 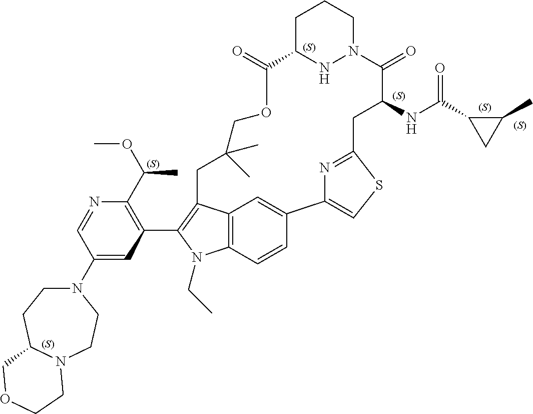 |
| DA269 | 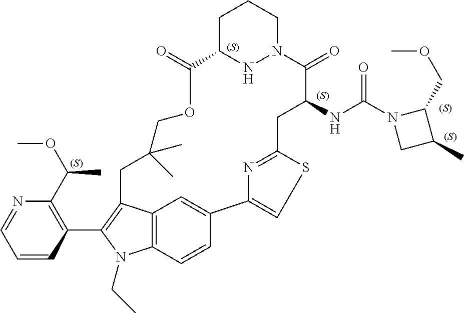 |
| DA270 | 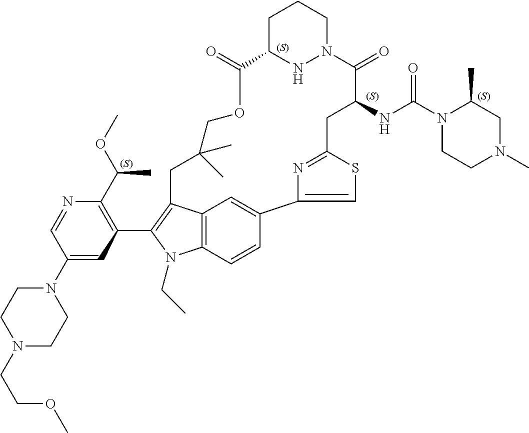 |
| DA271 | 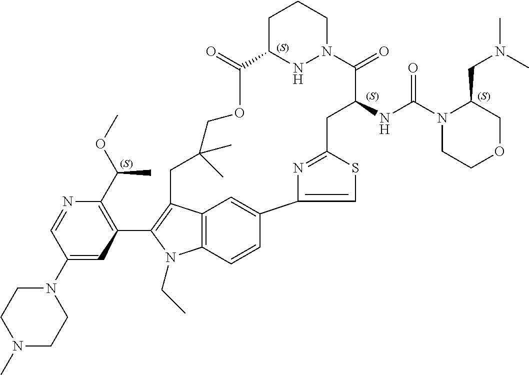 |
| DA272 | 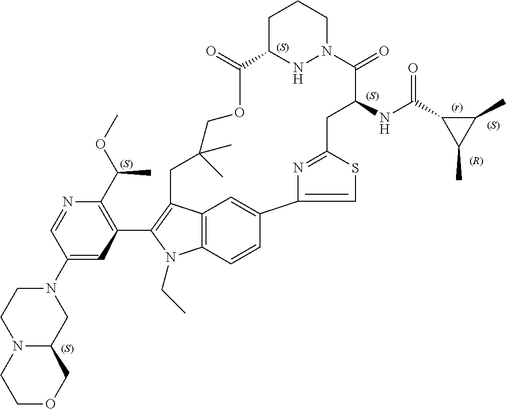 |
| DA273 | 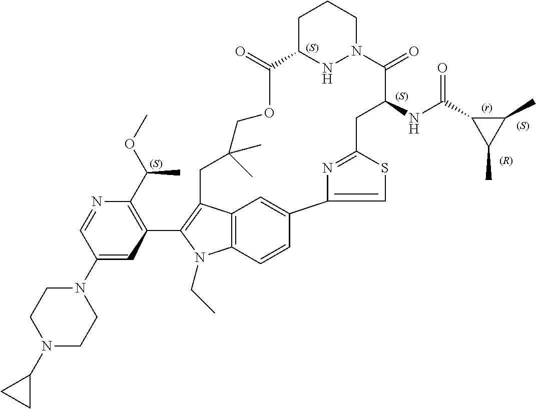 |
| DA274 | 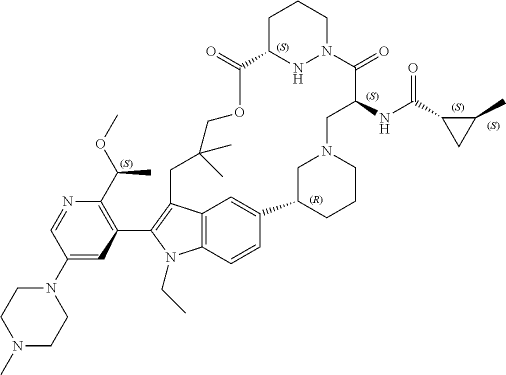 |
| DA275 | 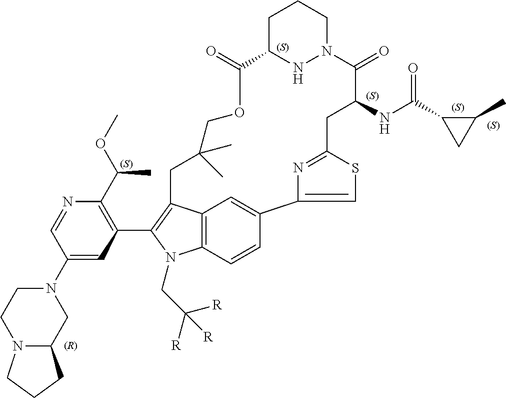 |
| DA276 | 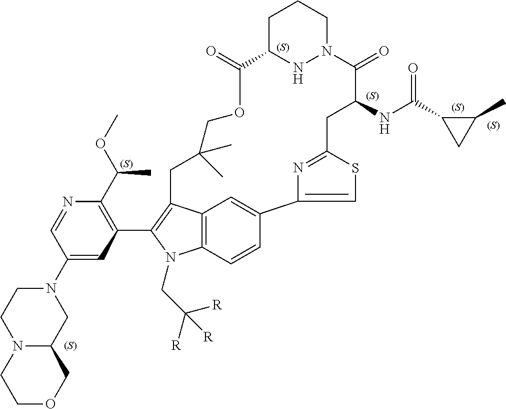 |
| DA277 | 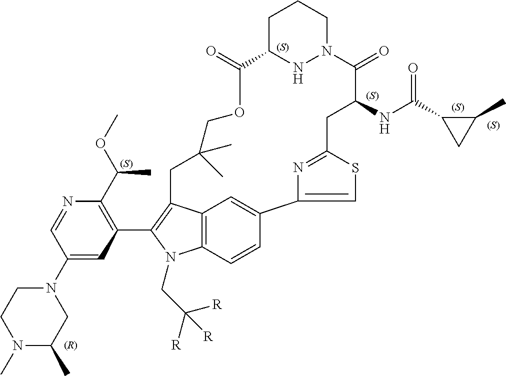 |
| DA278 | 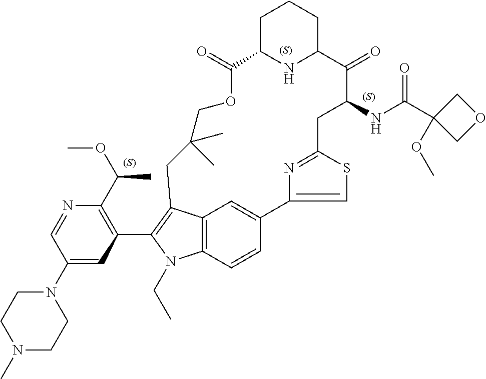 |
| DA279 | 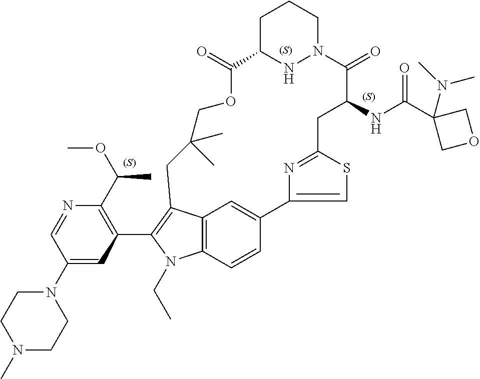 |
| DA280 | 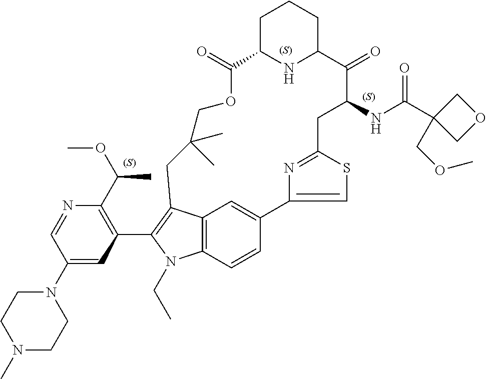 |
| DA281 | 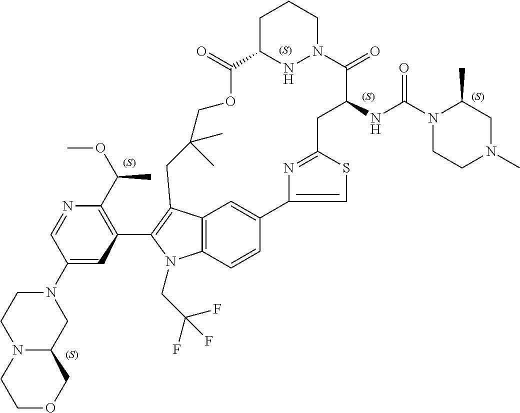 |
| DA282 | 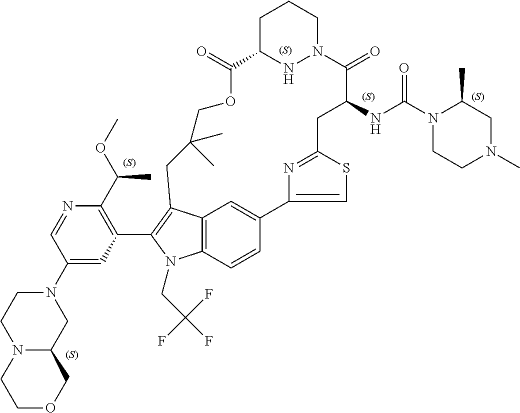 |
| DA283 | 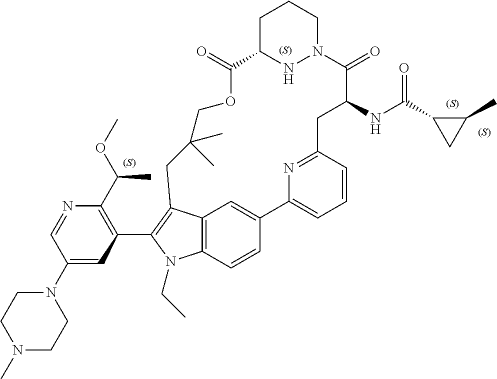 |
| DA284 | 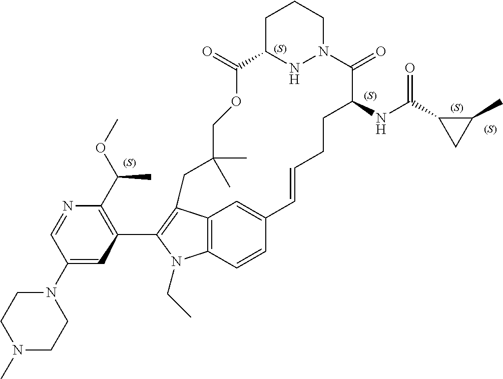 |
| DA285 | 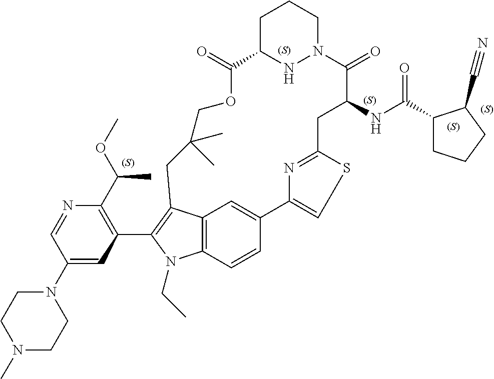 |
| DA286 | 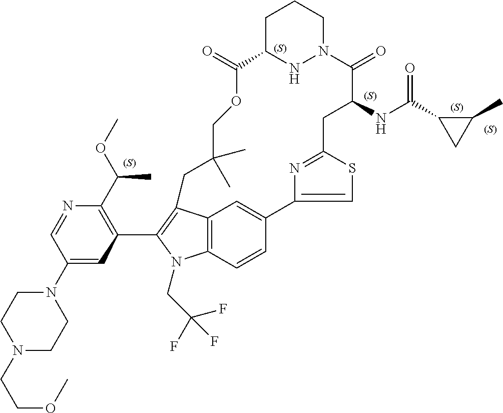 |
| DA287 | 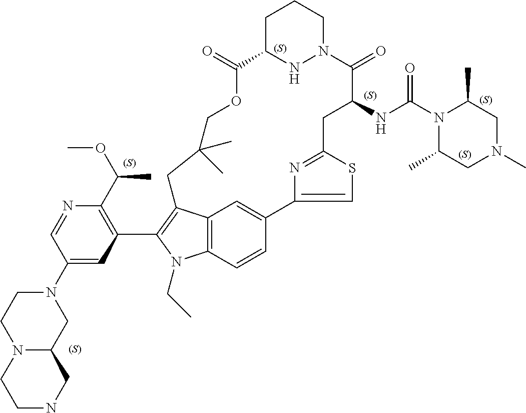 |
| DA288 | 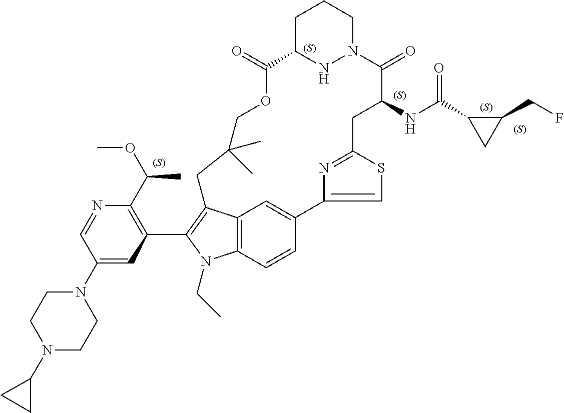 |
| DA289 | 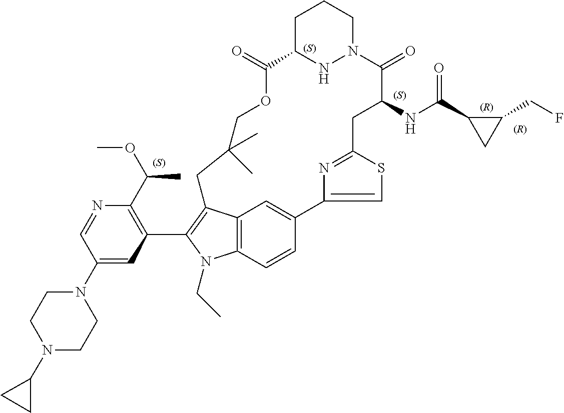 |
| DA290 | 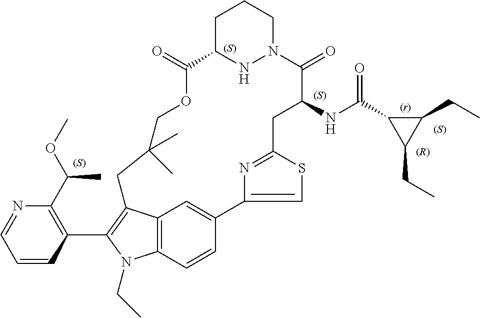 |
| DA291 | 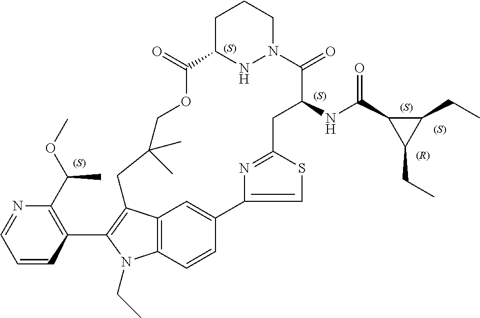 |
| DA292 | 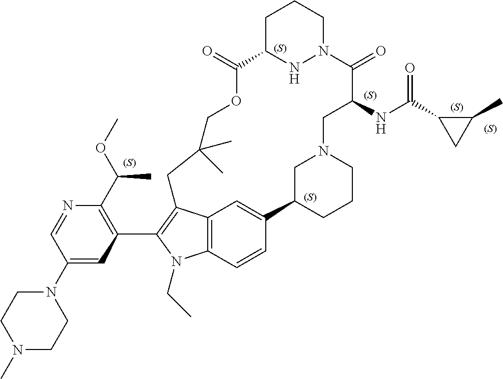 |
| DA293 | 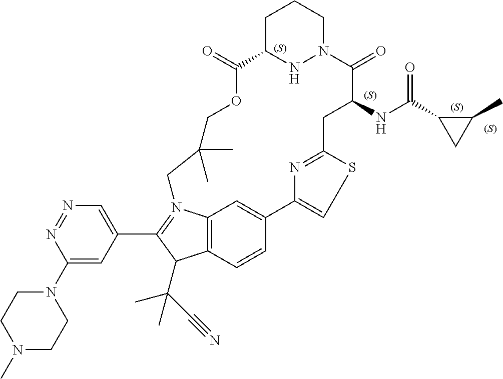 |
| DA294 | 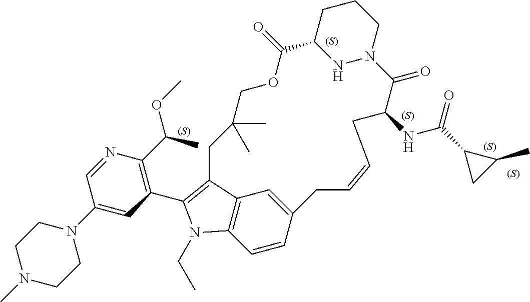 |
| DA295 | 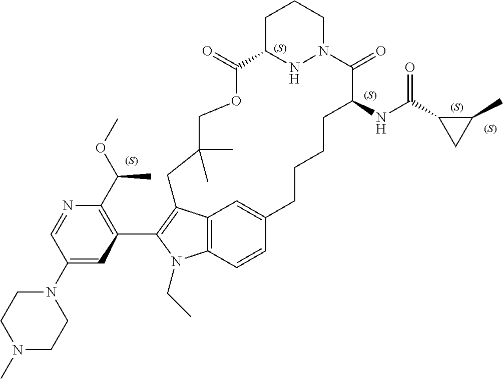 |
| DA296 | 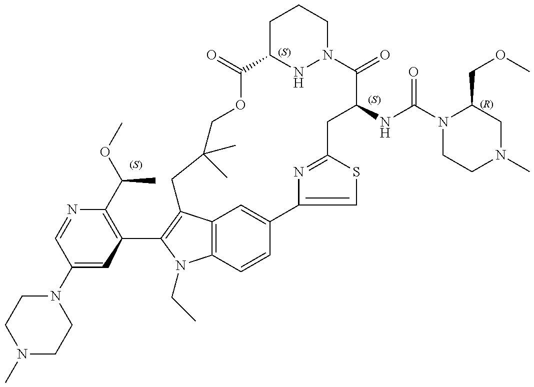 |
| DA297 | 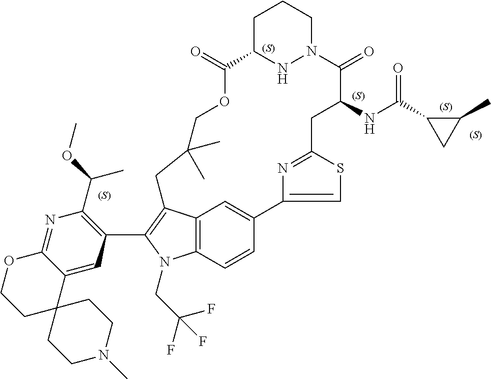 |
| DA298 | 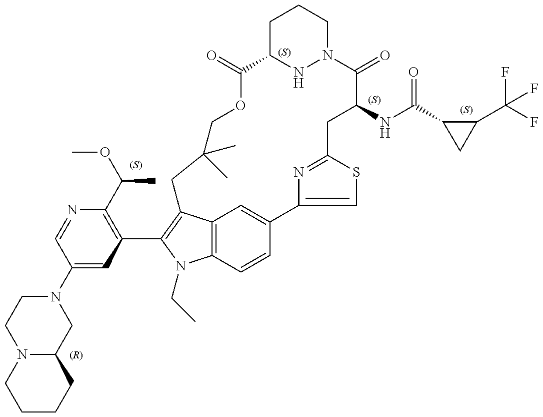 |
| DA299 | 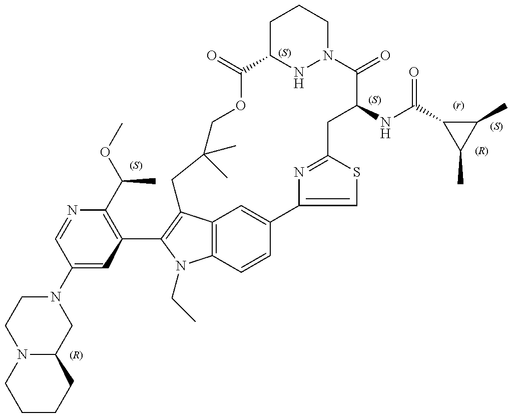 |
| DA300 | 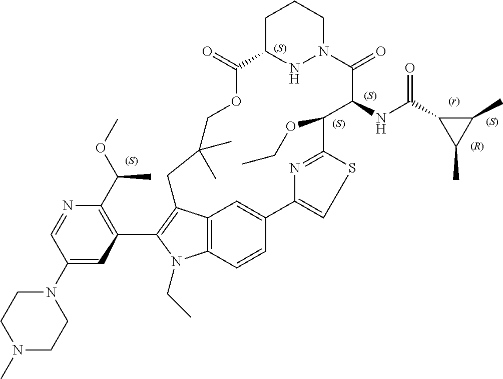 |
| DA301 | 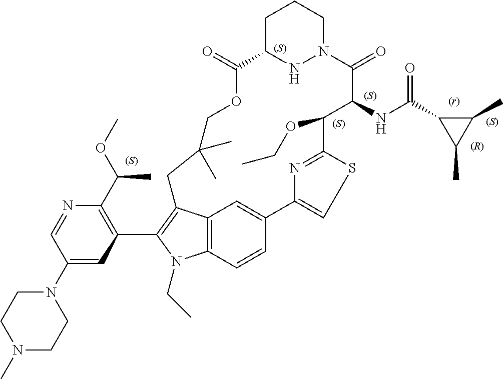 |
| DA302 | 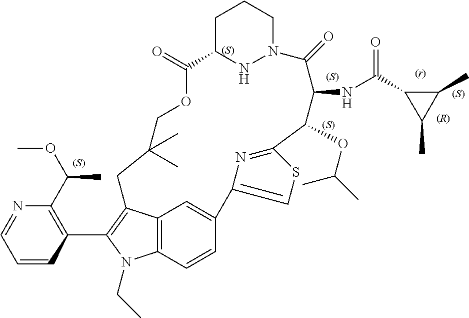 |
| DA303 | 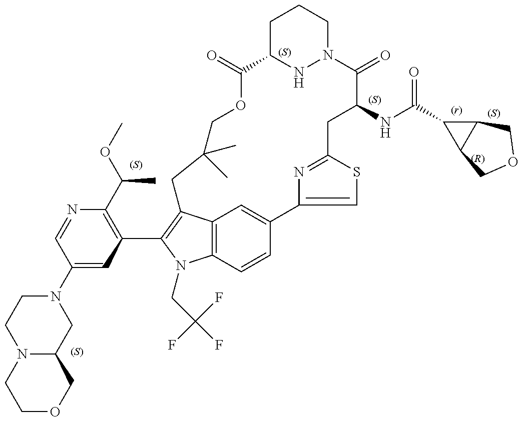 |
| DA304 | 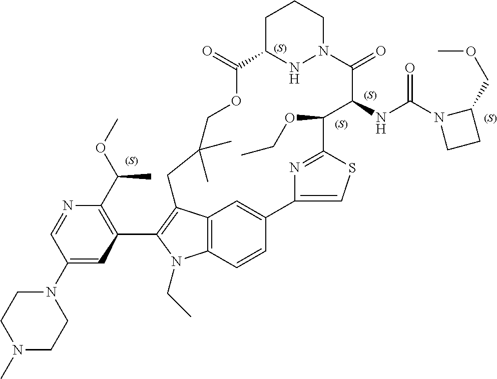 |
| DA305 | 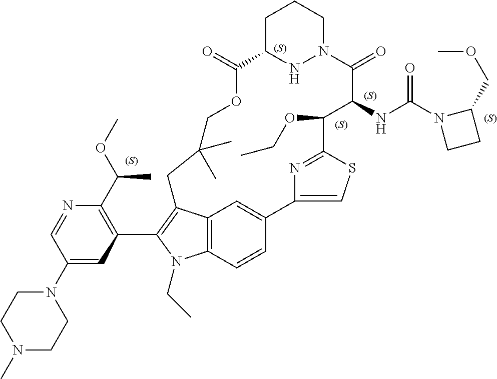 |
| DA306 | 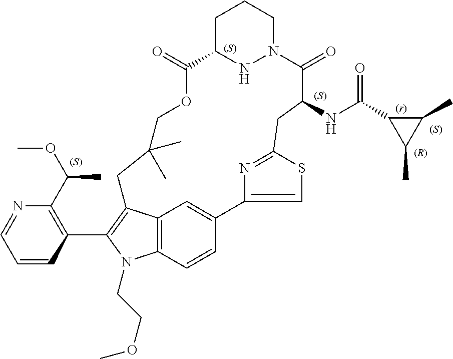 |
| DA307 | 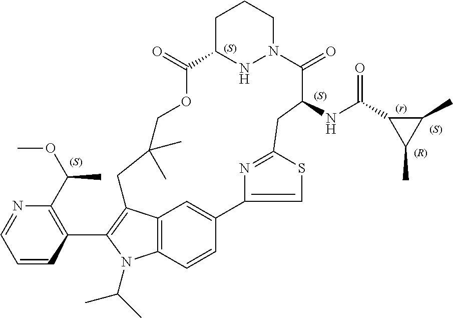 |
| DA308 | 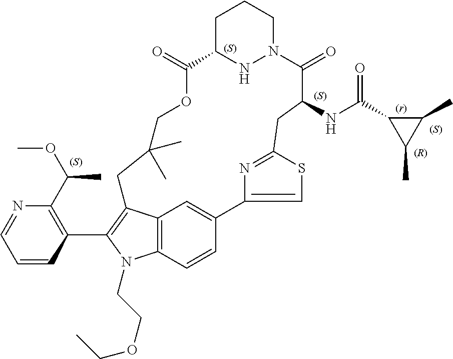 |
| DA309 | 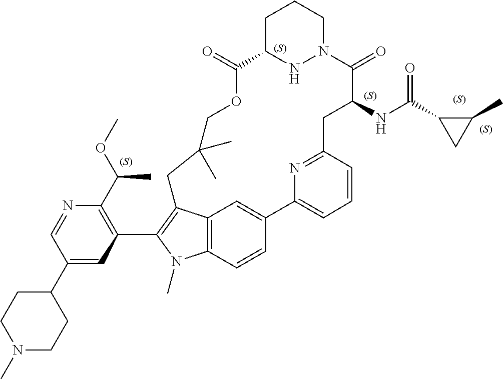 |
| DA310 | 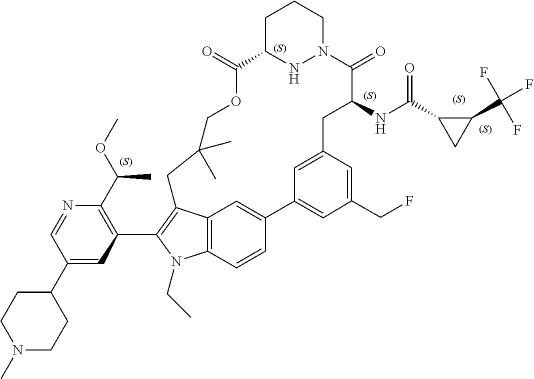 |
| DA311 | 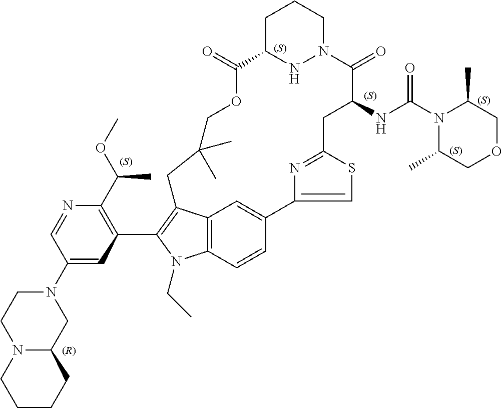 |
| DA312 | 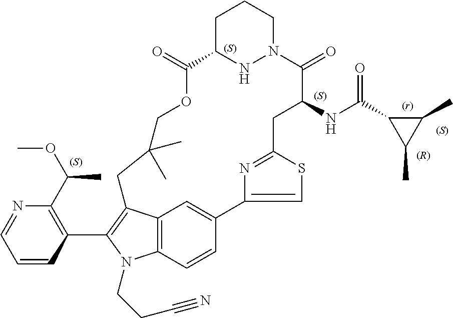 |
| DA313 | 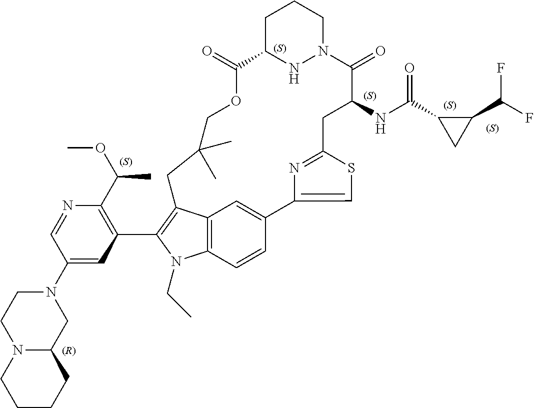 |
| DA314 | 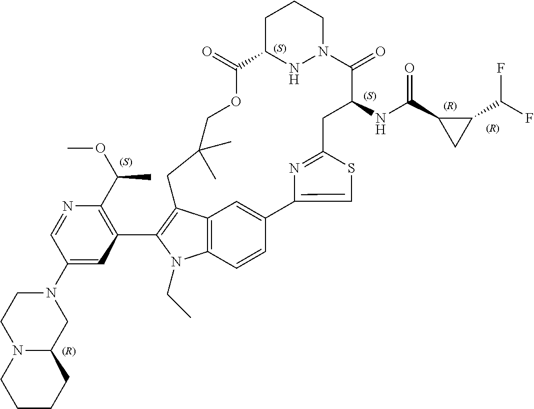 |
| DA315 | 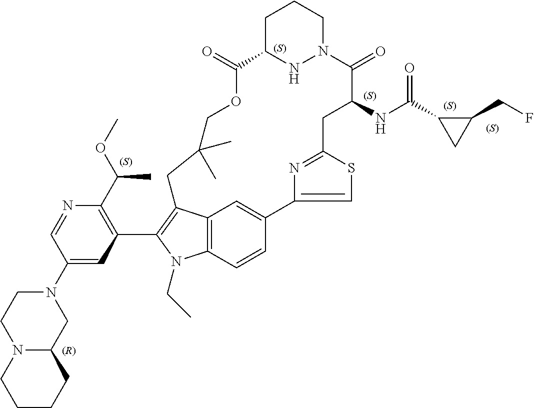 |
| DA316 | 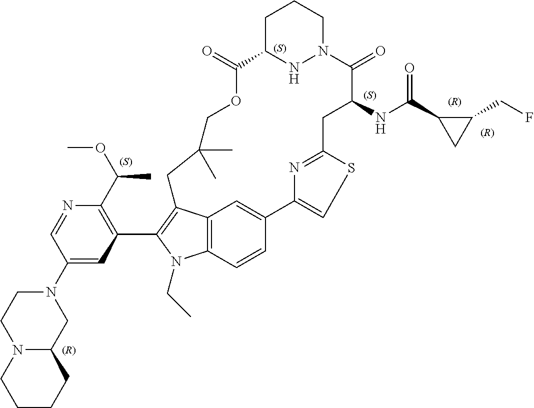 |
| DA317 | 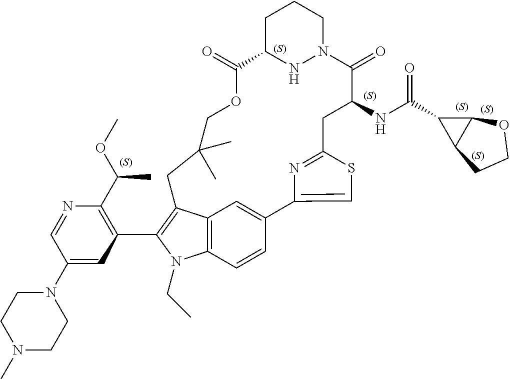 |
| DA318 | 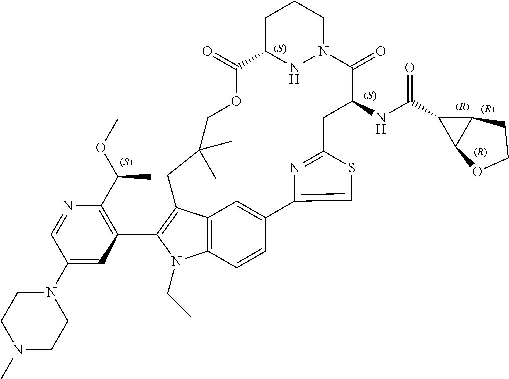 |
| DA319 | 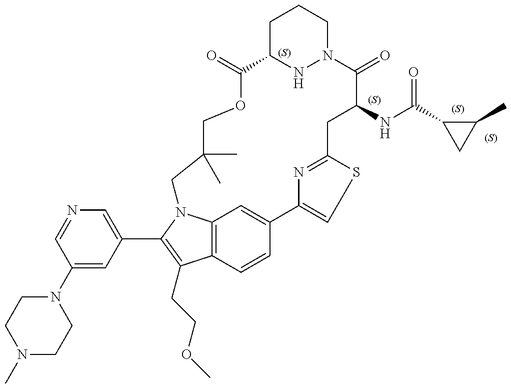 |
| DA320 | 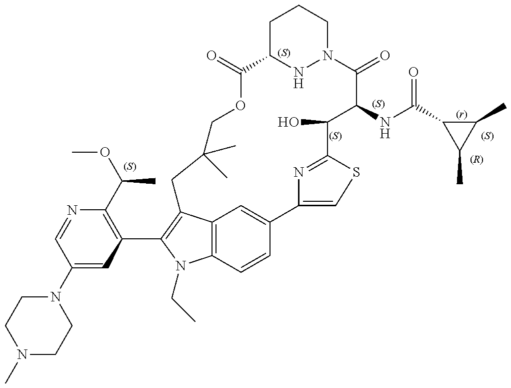 |
| DA321 | 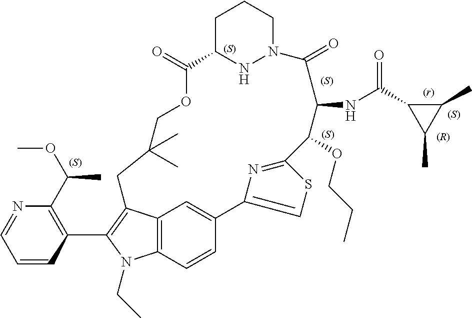 |
| DA322 | 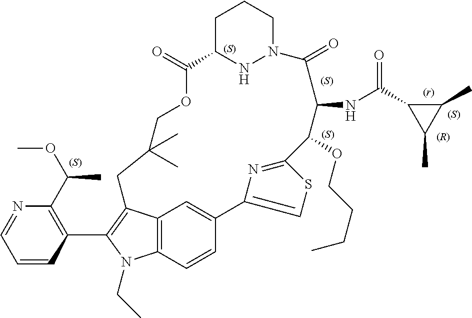 |
| DA323 | 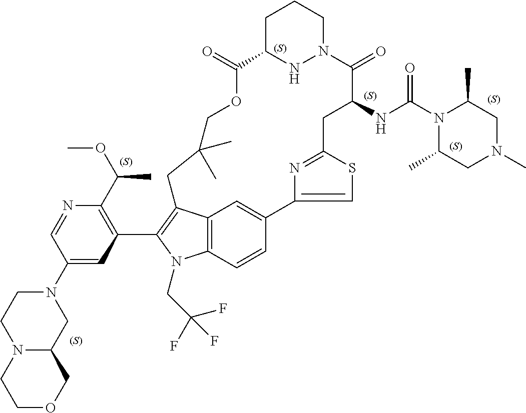 |
| DA324 | 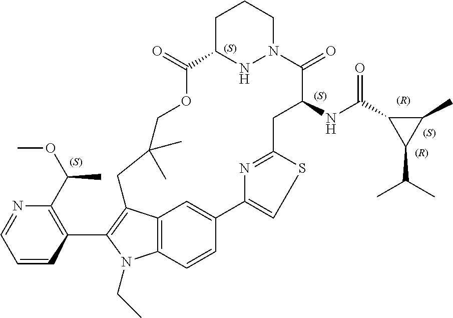 |
| DA325 | 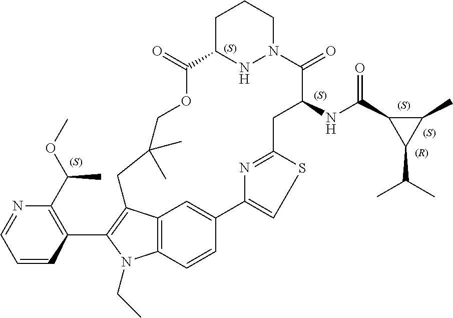 |
| DA326 | 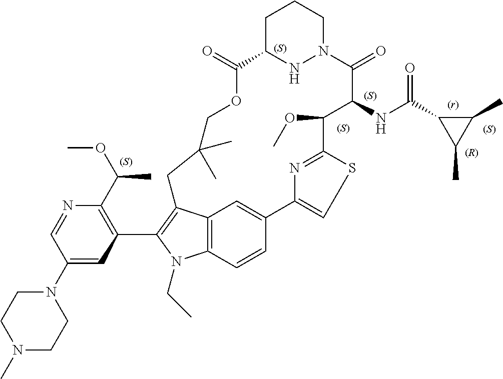 |
| DA327 | 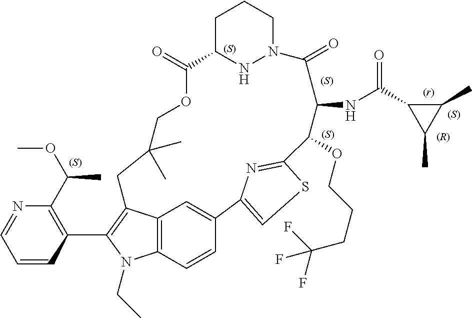 |
| DA328 | 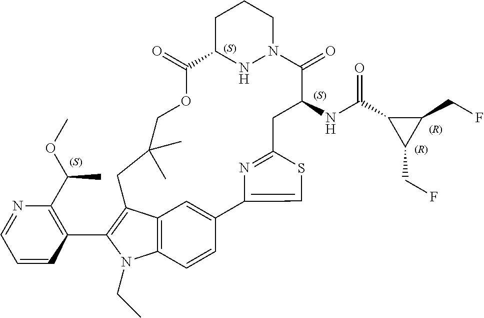 |
| DA329 | 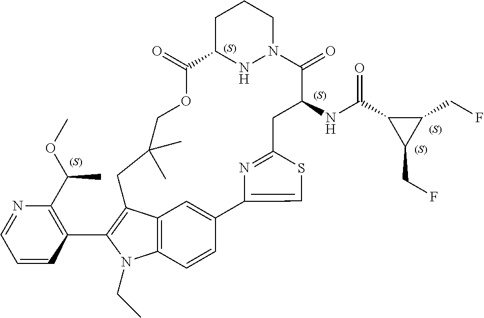 |
| DA330 | 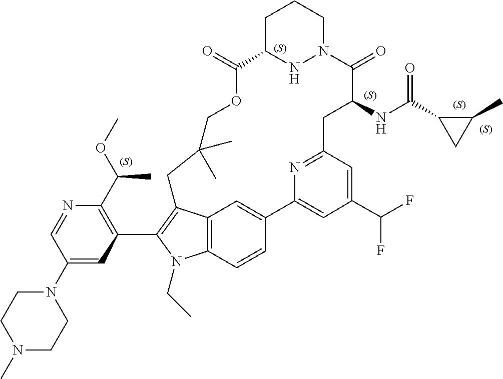 |
| DA331 | 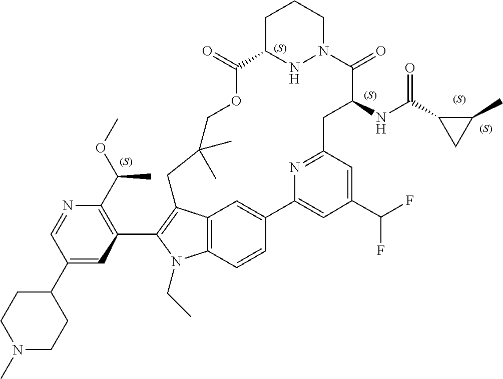 |
| DA332 | 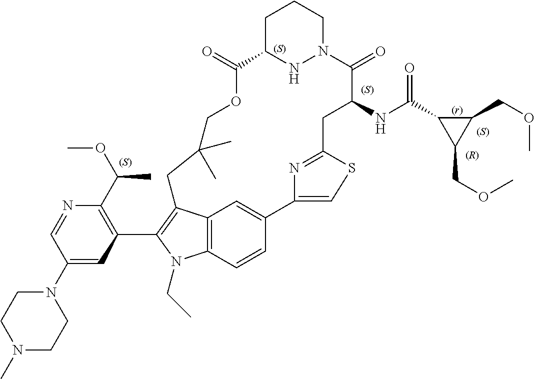 |
| DA333 | 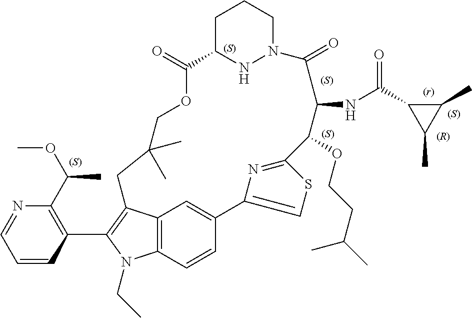 |
| DA334 | 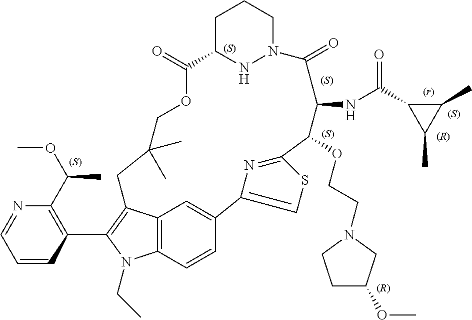 |
| DA335 | 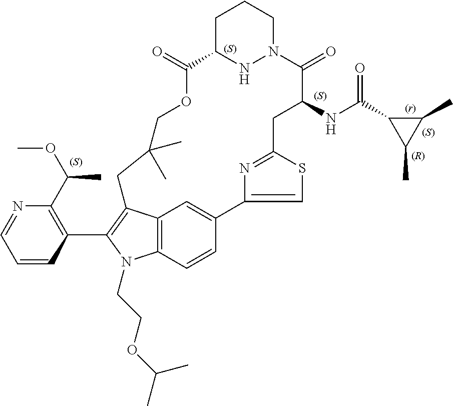 |
| DA336 | 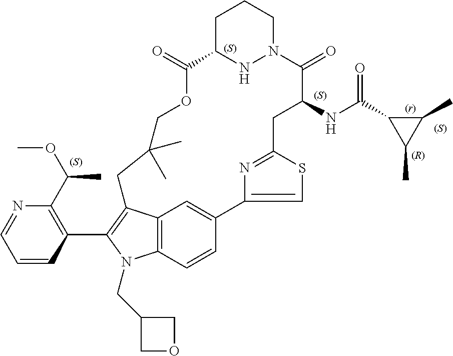 |
| DA337 | 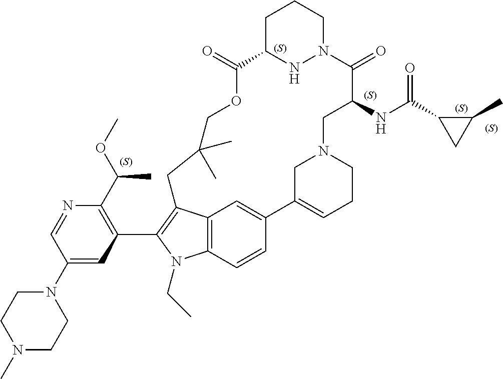 |
| DA338 | 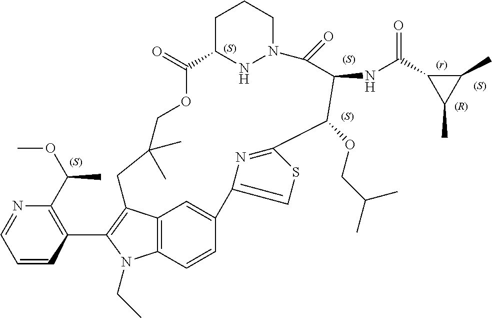 |
| DA339 | 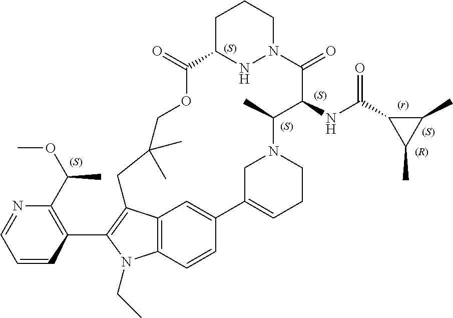 |
| DA340 | 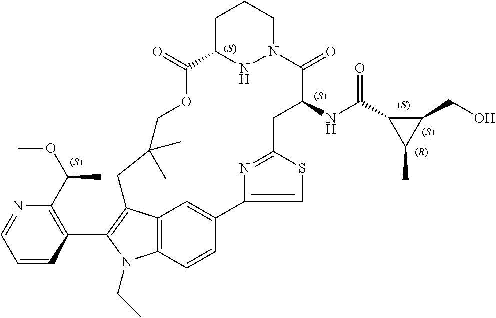 |
| DA341 | 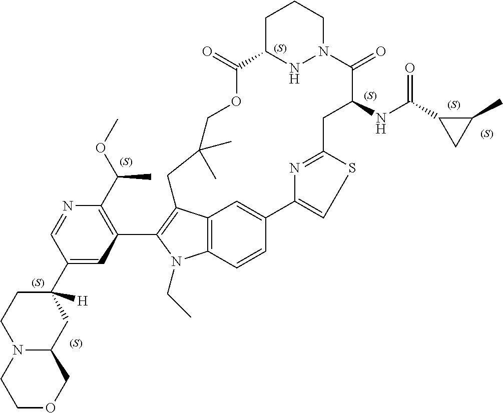 |
| DA342 | 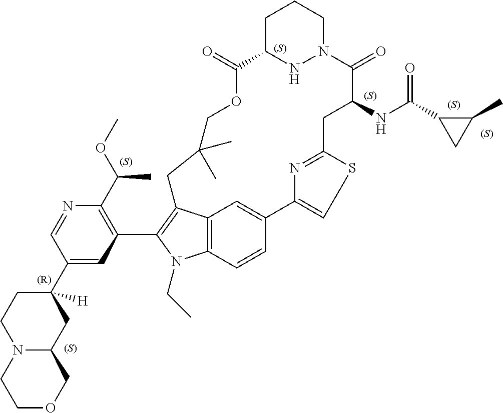 |
| DA343 | 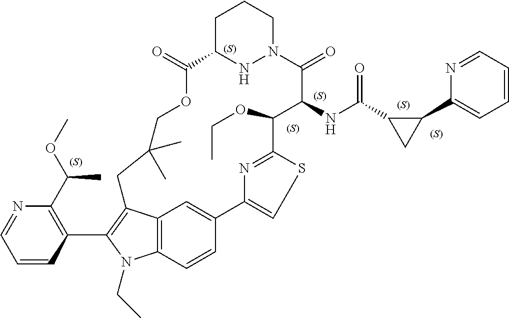 |
| DA344 | 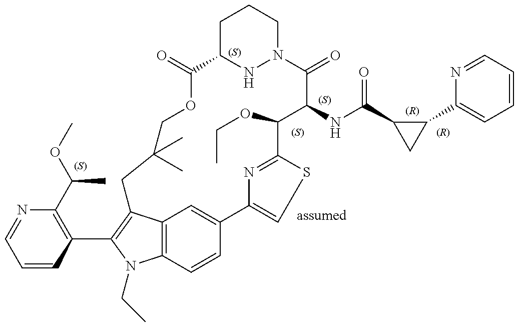 |
| DA345 | 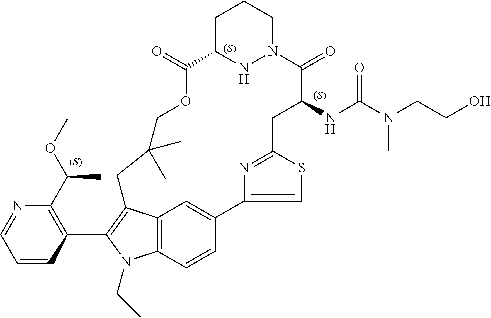 |
| DA346 | 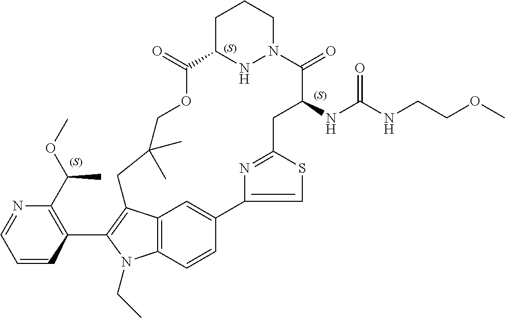 |
| DA347 | 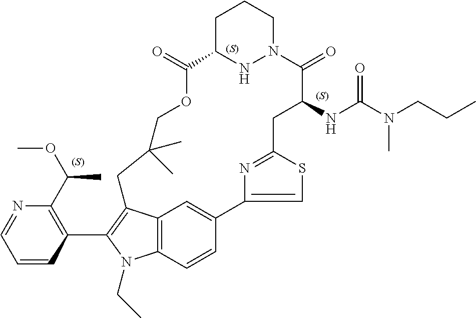 |
| DA348 | 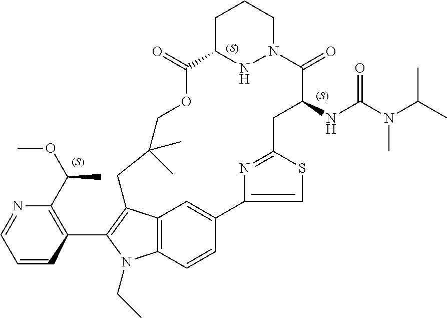 |
| DA349 | 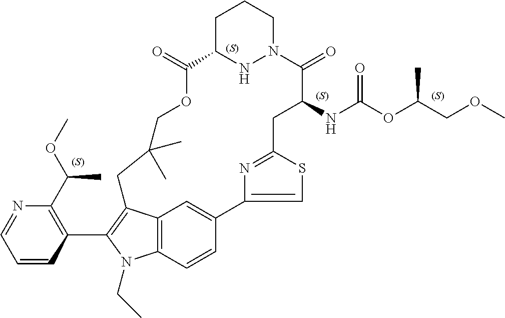 |
| DA350 | 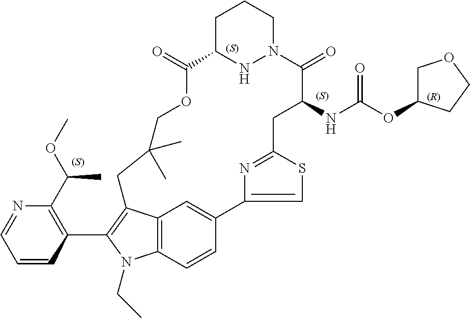 |
| DA351 | 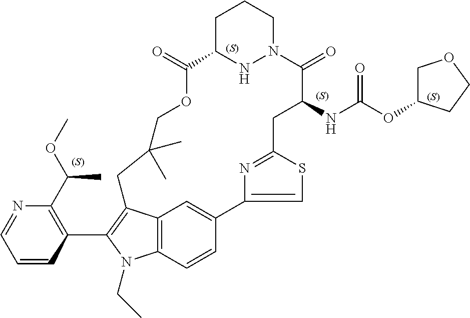 |
| DA352 | 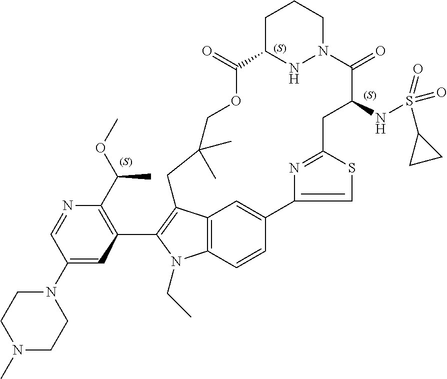 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

In some embodiments, the RAS(ON) inhibitor is selected from Table D1 b, or a pharmaceutically acceptable salt or stereoisomer thereof. In some embodiments, the RAS(ON) inhibitor is selected from Table D1 b, or a pharmaceutically acceptable salt or atropisomer thereof.

TABLE D1b
Certain Compounds of the Present Invention

| Ex. # | Structure |
|---|---|
| DA354 | 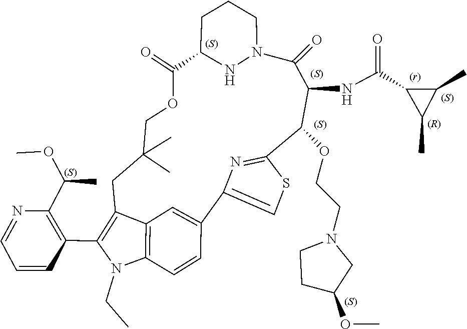 |
| DA355 | 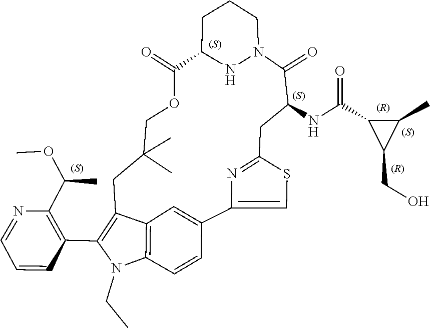 |
| DA356 | 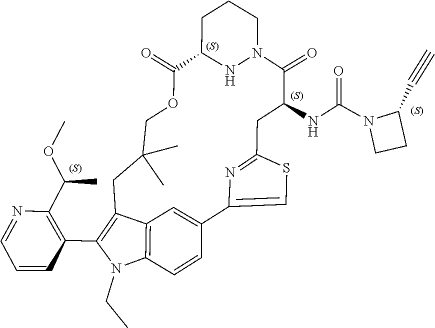 |
| DA357 | 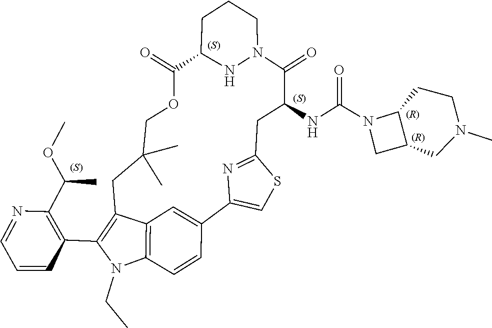 |
| DA358 | 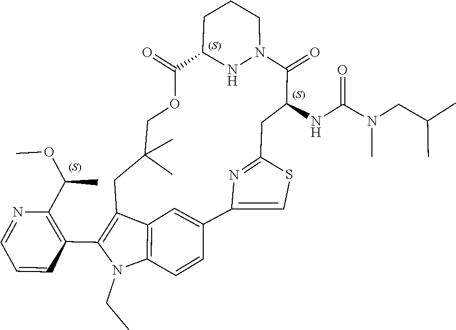 |
| DA359 | 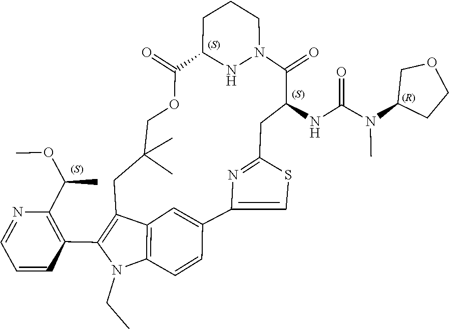 |
| DA360 | 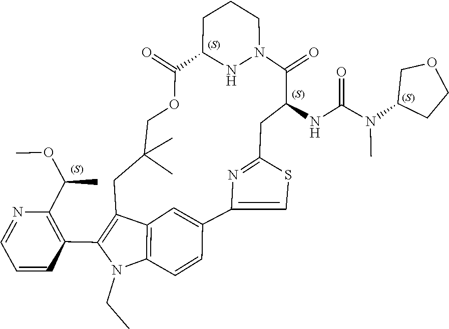 |
| DA361 | 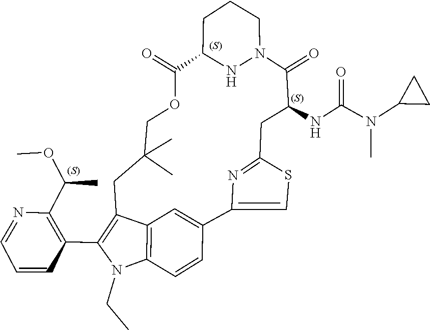 |
| DA362 | 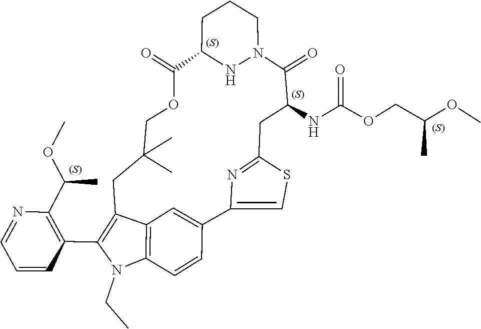 |
| DA363 | 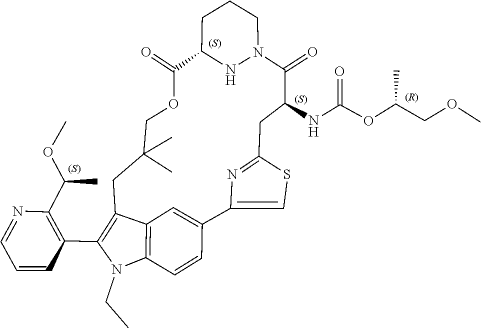 |
| DA364 | 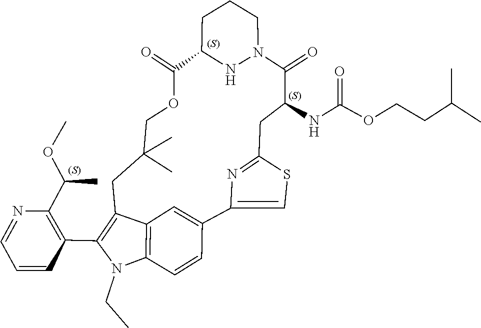 |
| DA365 | 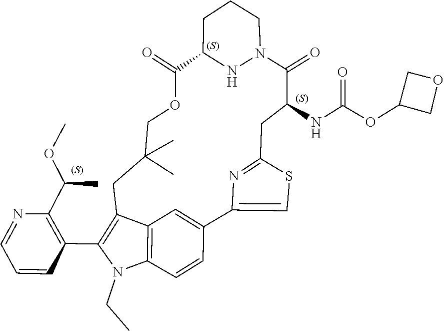 |
| DA366 | 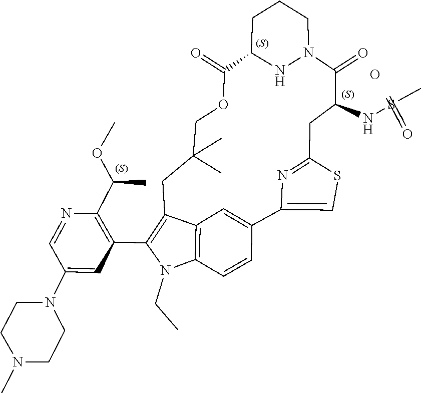 |
| DA367 | 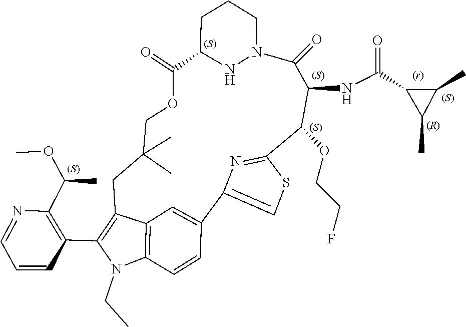 |
| DA3658 | 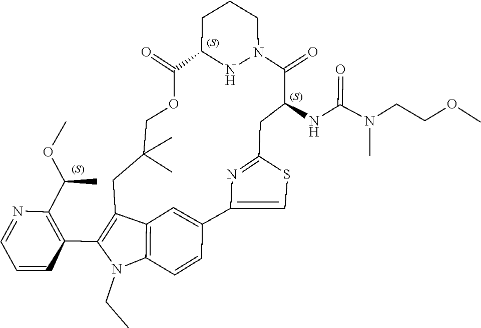 |
| DA369 | 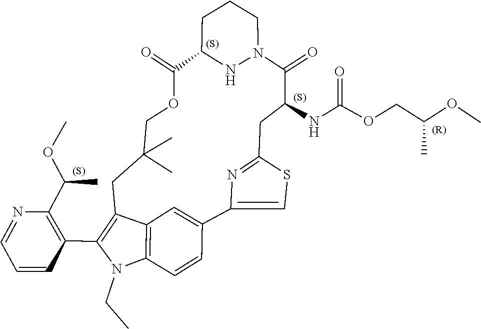 |
| DA370 | 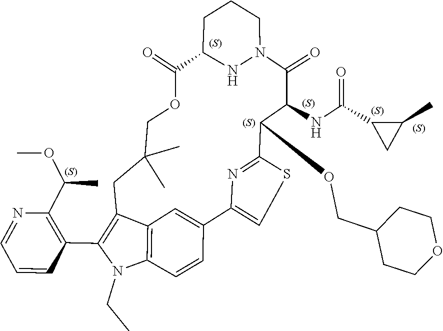 |
| DA371 | 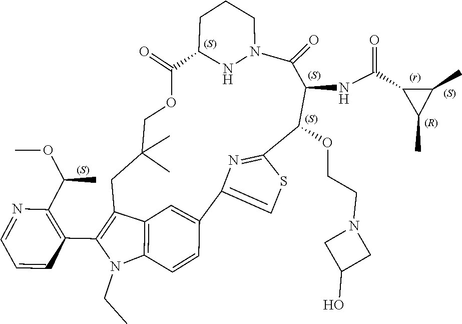 |
| DA372 | 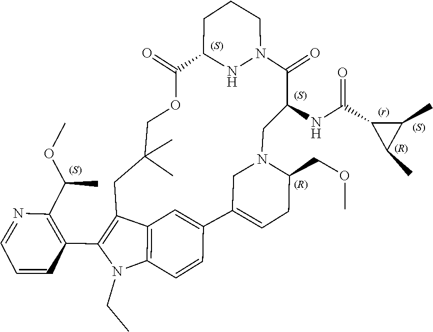 |
| DA373 | 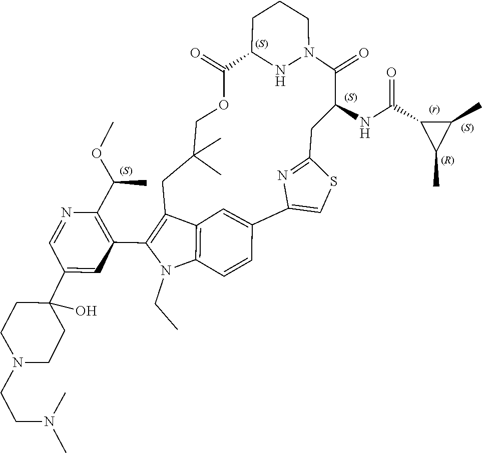 |
| DA374 | 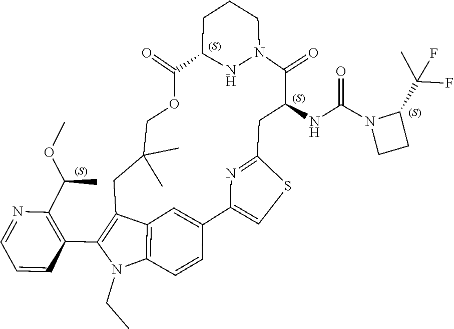 |
| DA375 | 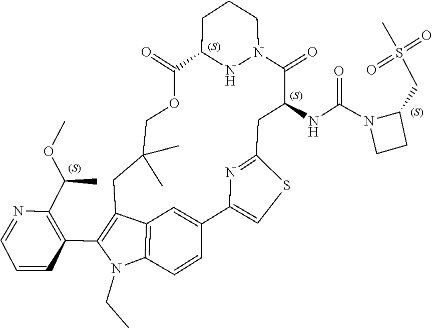 |
| DA376 | 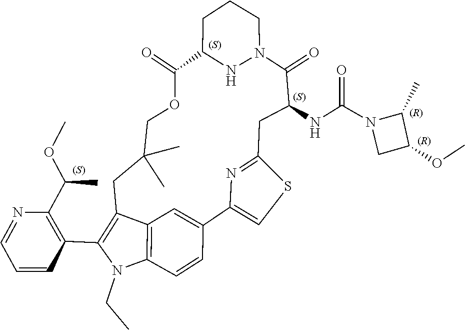 |
| DA377 | 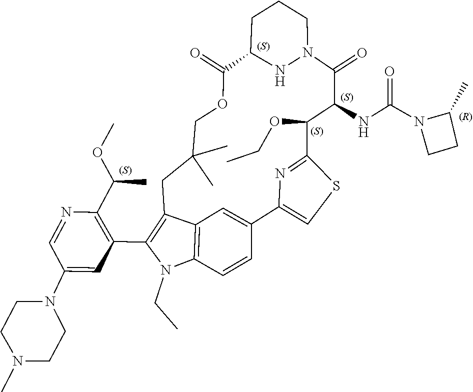 |
| DA378 | 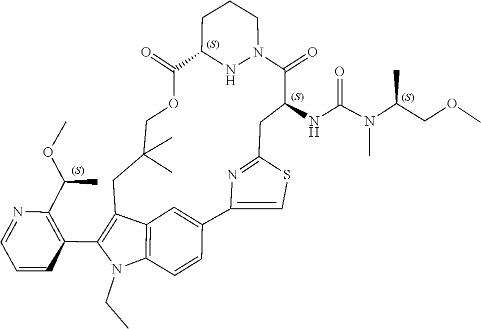 |
| DA379 | 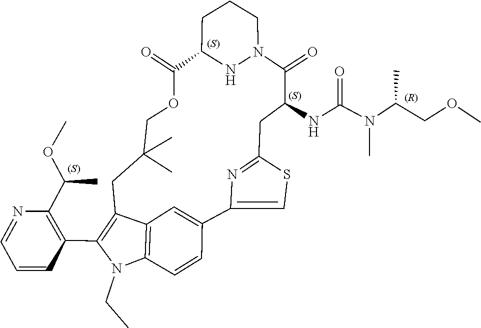 |
| DA380 | 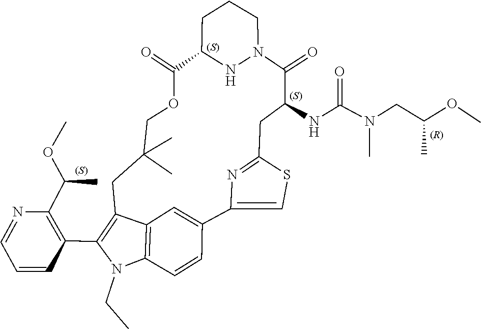 |
| DA381 | 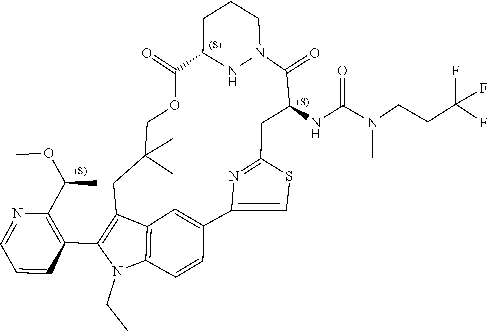 |
| DA382 | 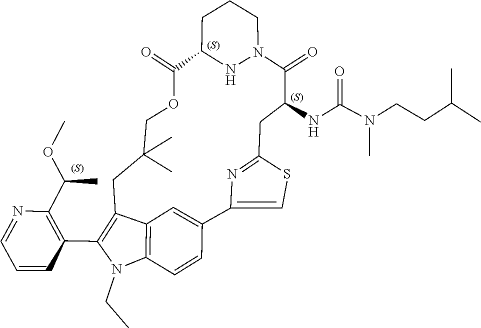 |
| DA383 | 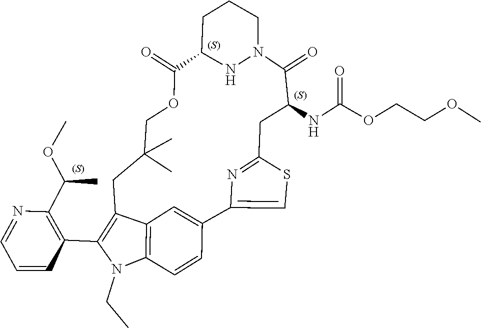 |
| DA384 | 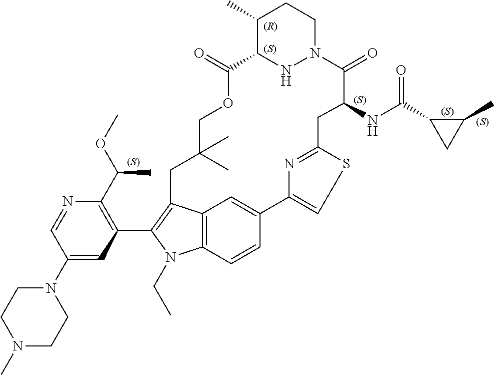 |
| DA385 | 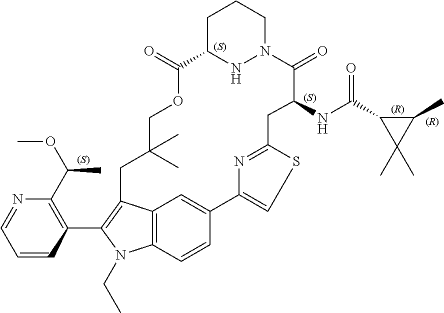 |
| DA386 | 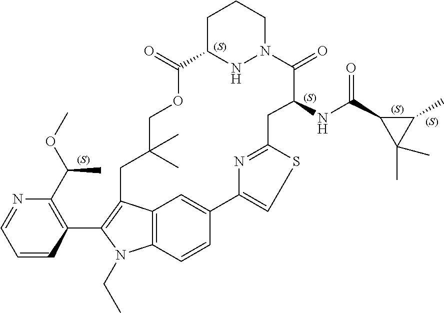 |
| DA387 | 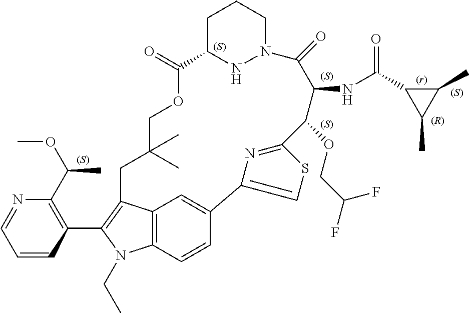 |
| DA388 | 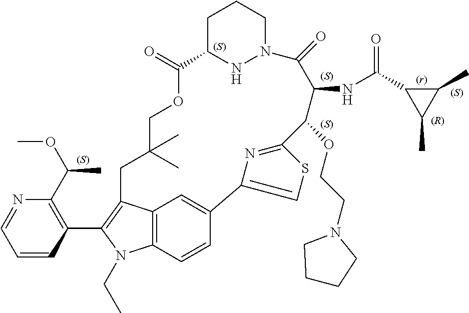 |
| DA389 | 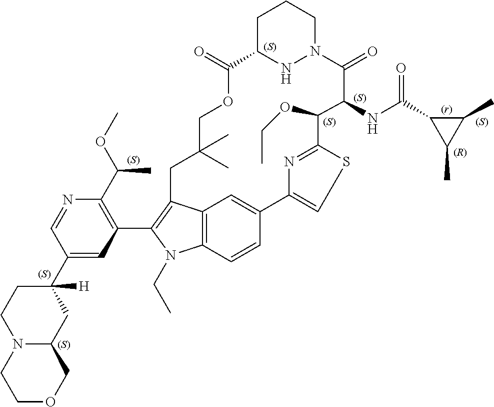 |
| DA390 | 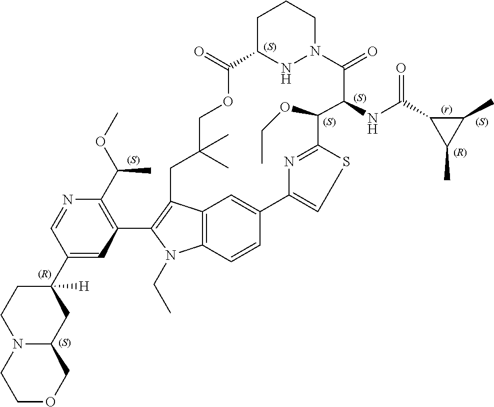 |
| DA391 | 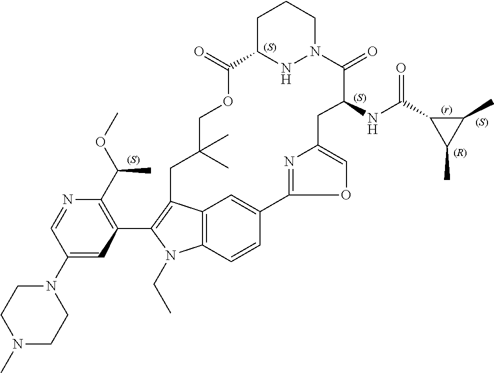 |
| DA392 | 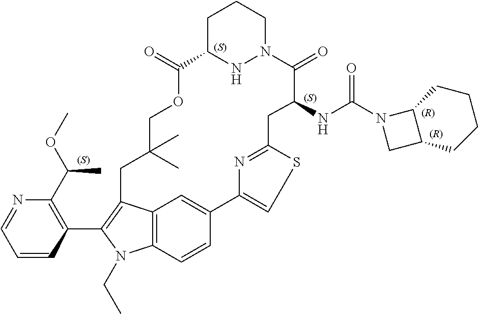 |
| DA393 | 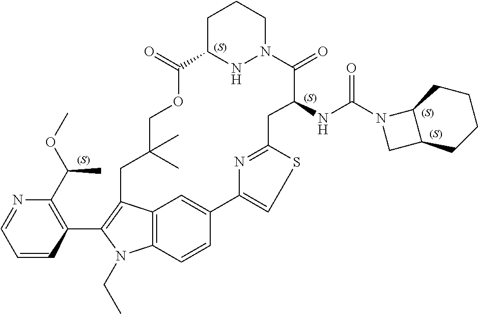 |
| DA394 | 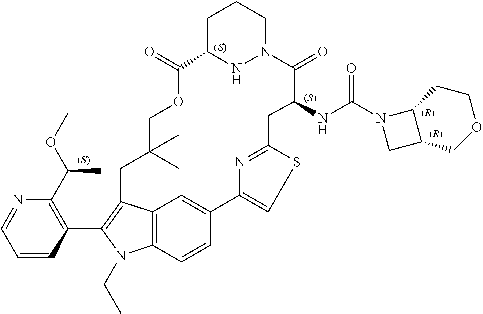 |
| DA395 | 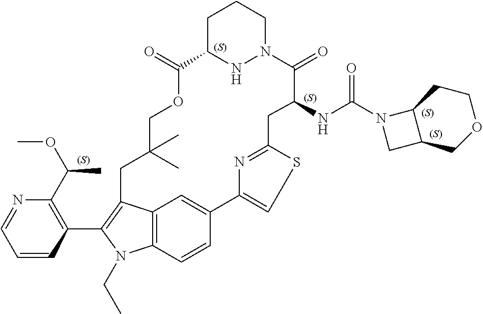 |
| DA396 | 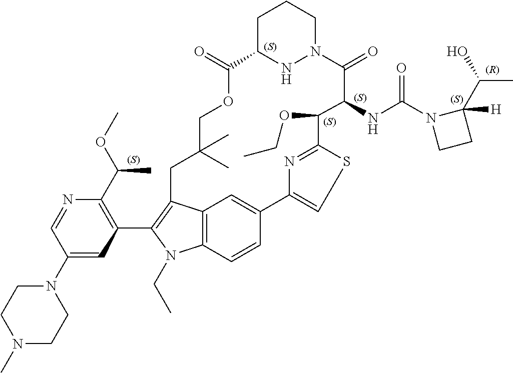 |
| DA397 | 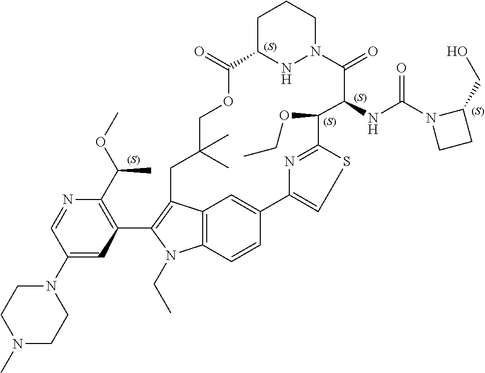 |
| DA398 | 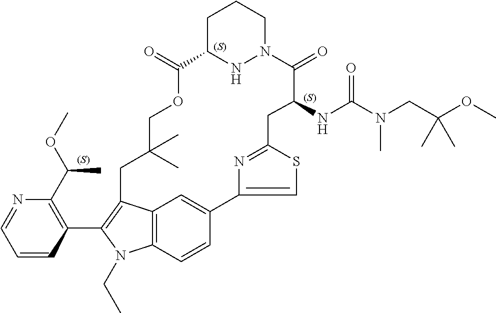 |
| DA399 | 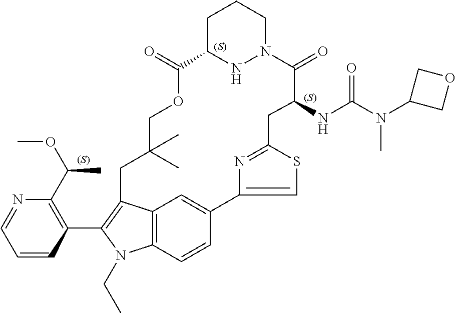 |
| DA400 | 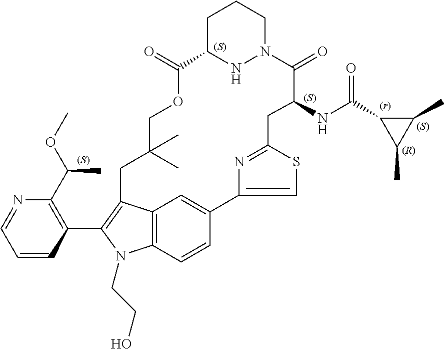 |
| DA401 | 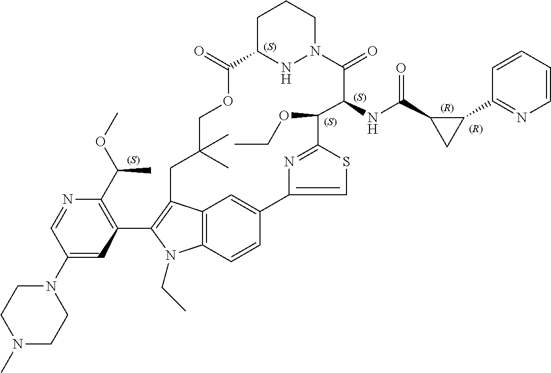 |
| DA402 | 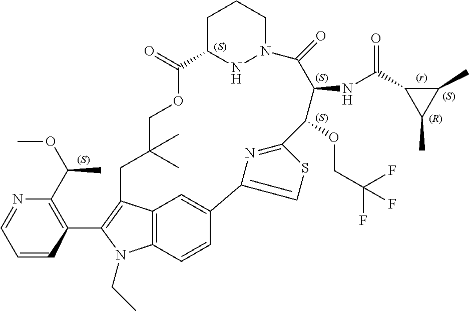 |
| DA403 | 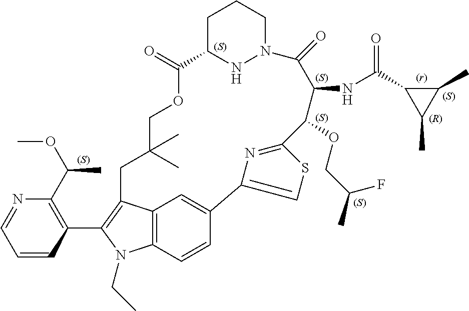 |
| DA404 | 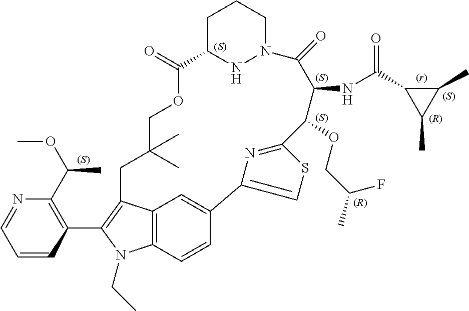 |
| DA405 | 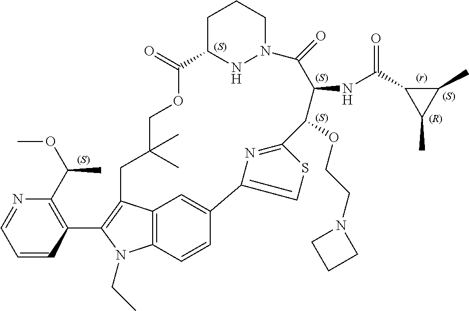 |
| DA406 | 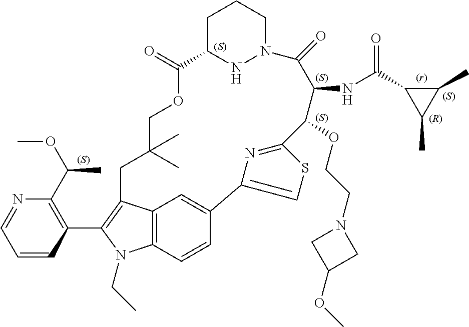 |
| DA407 | 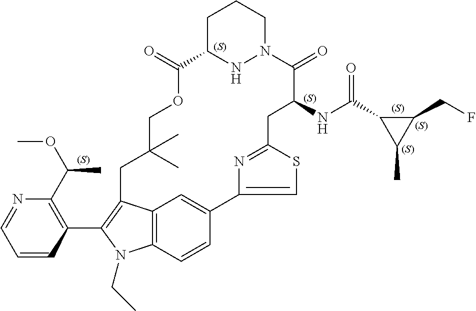 |
| DA408 | 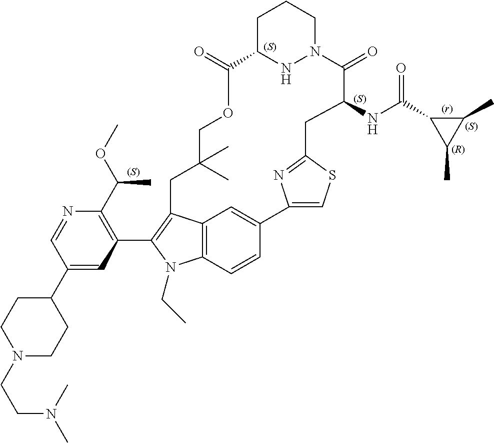 |
| DA409 | 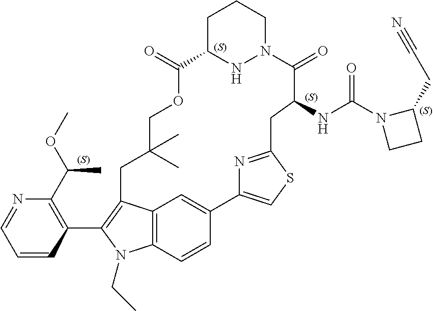 |
| DA410 | 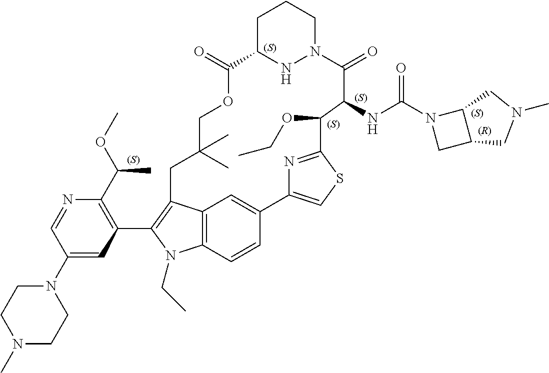 |
| DA411 | 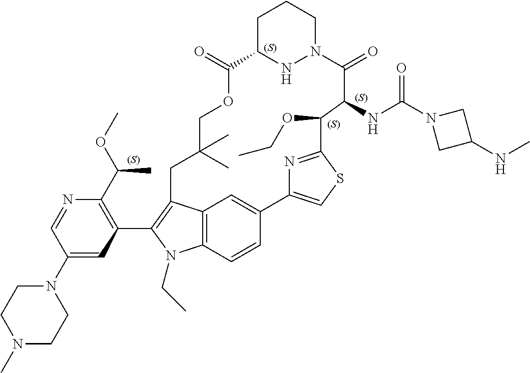 |
| DA412 | 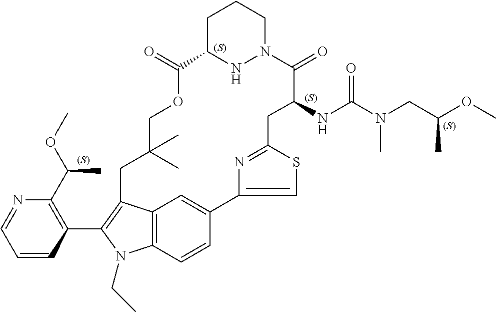 |
| DA413 | 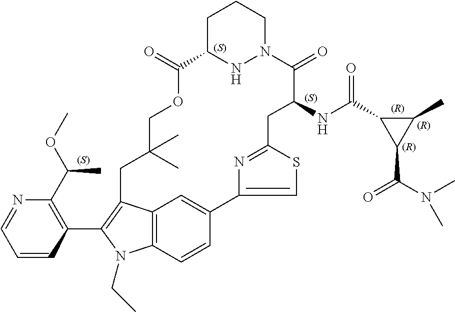 |
| DA414 | 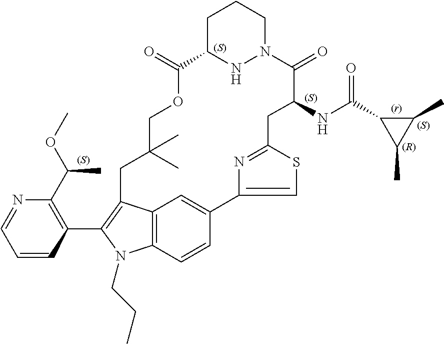 |
| DA415 | 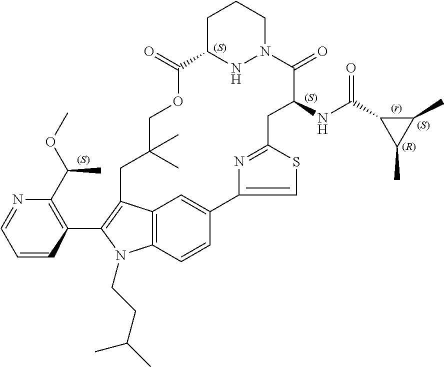 |
| DA416 | 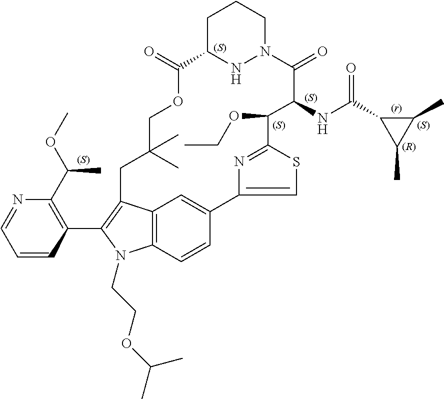 |
| DA417 | 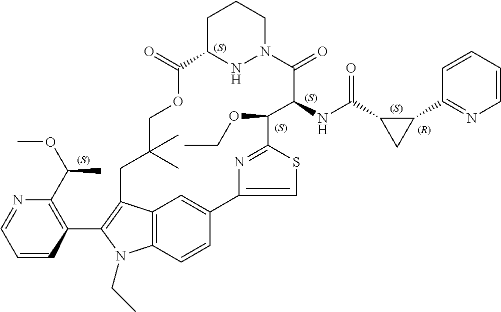 |
| DA418 | 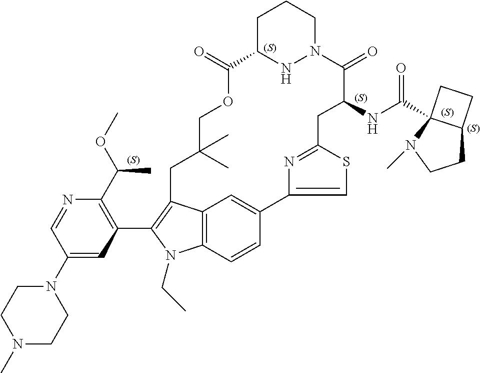 |
| DA419 | 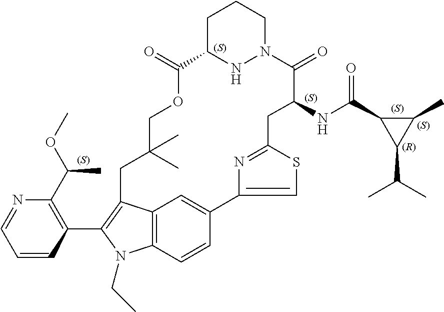 |
| DA420 | 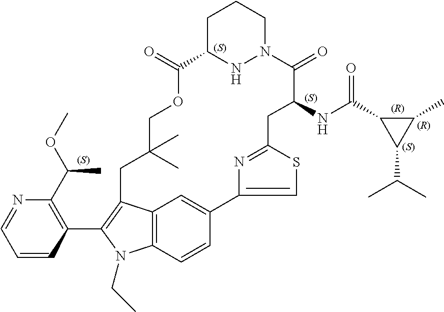 |
| DA421 | 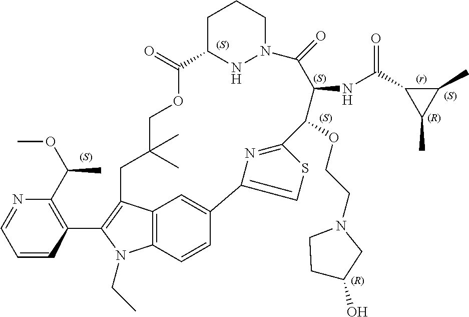 |
| DA422 | 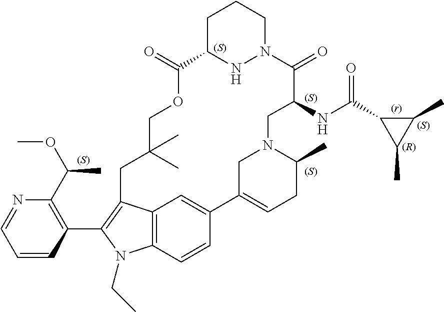 |
| DA423 | 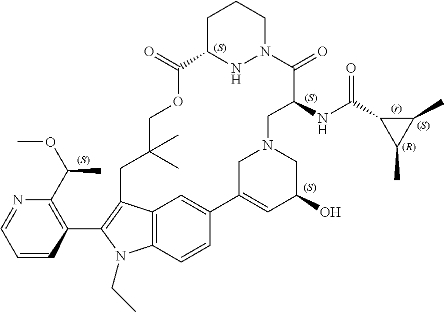 |
| DA424 | 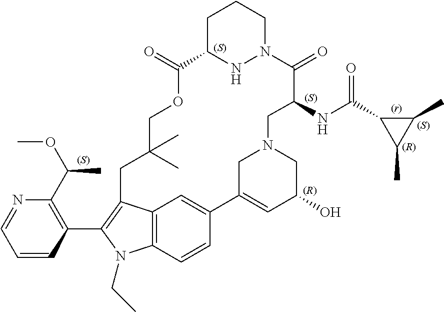 |
| DA425 | 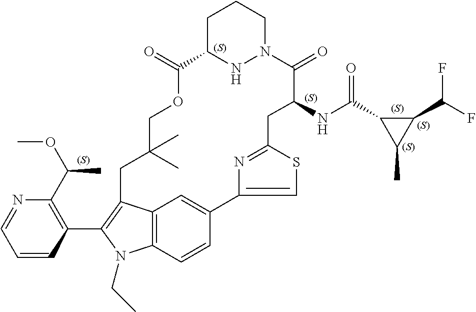 |
| DA426 | 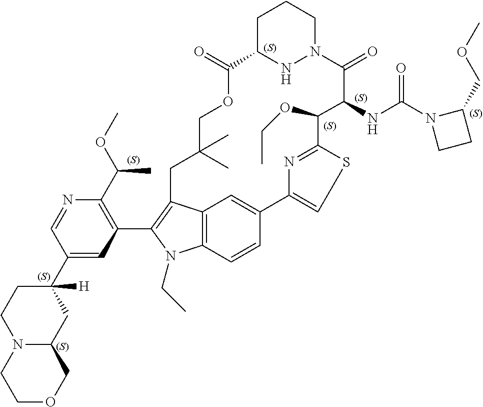 |
| DA427 | 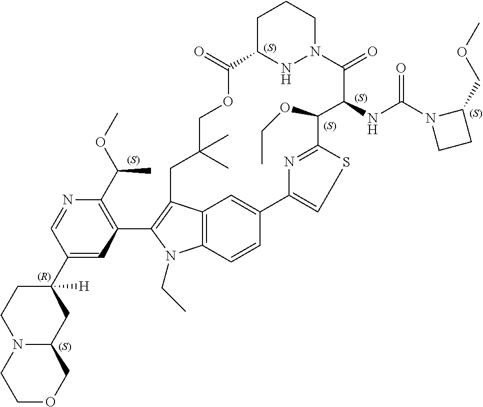 |
| DA428 | 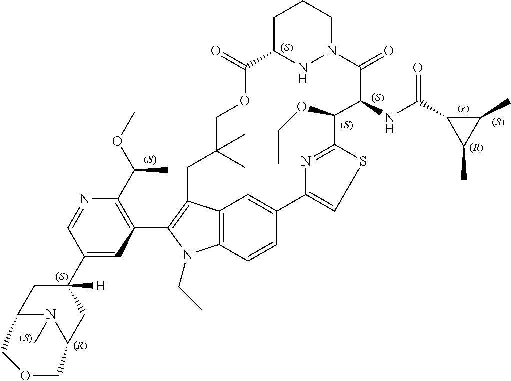 |
| DA429 | 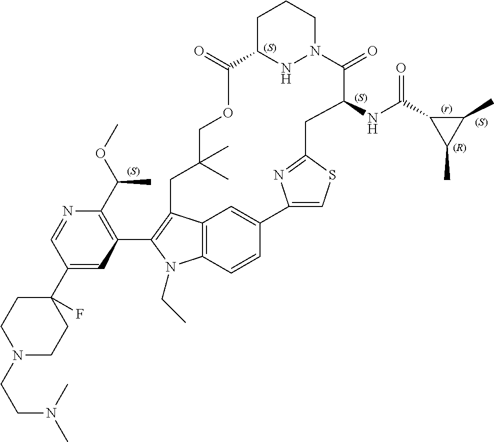 |
| DA430 | 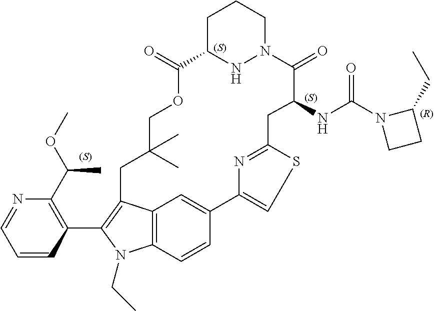 |
| DA431 | 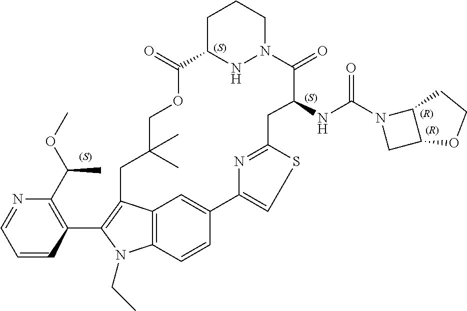 |
| DA432 | 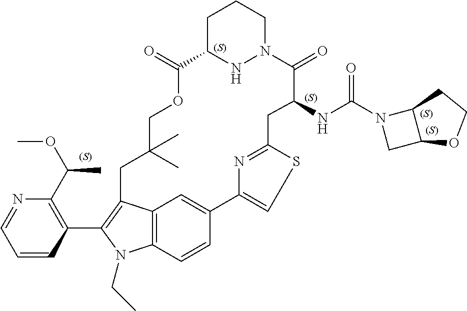 |
| DA433 | 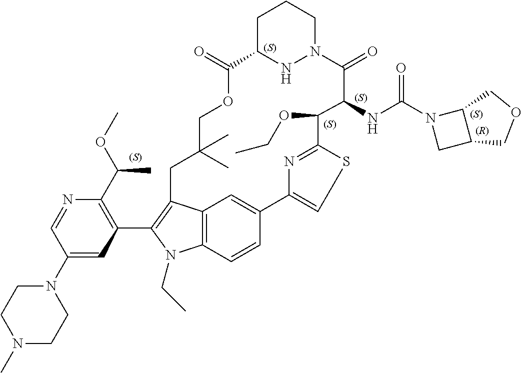 |
| DA434 | 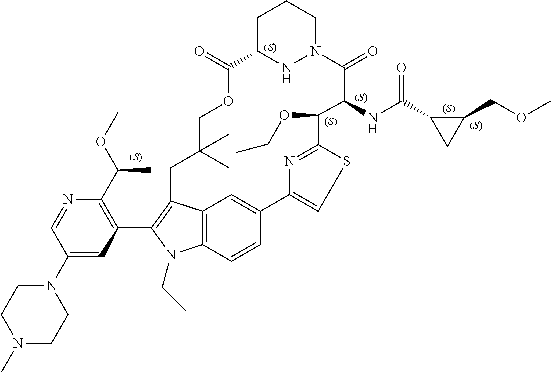 |
| DA435 | 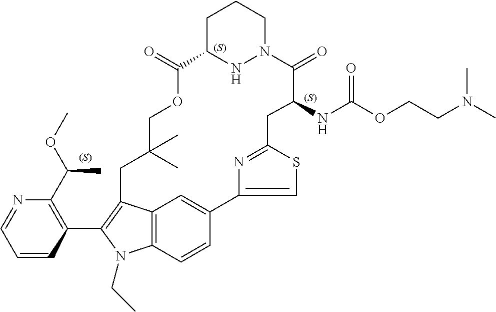 |
| DA436 | 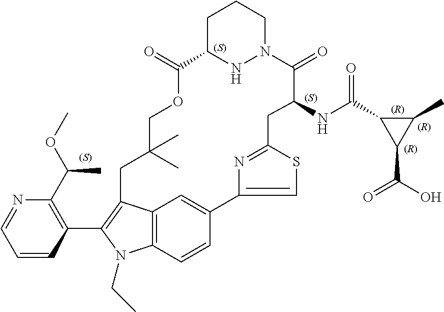 |
| DA437 | 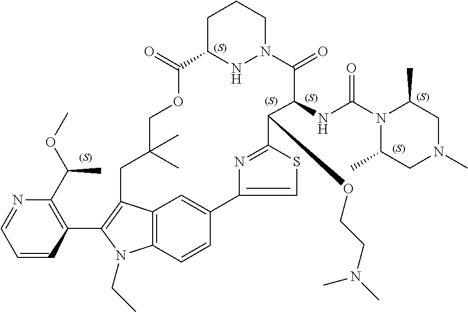 |
| DA438 | 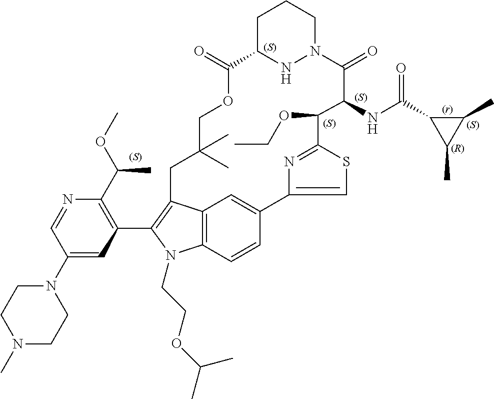 |
| DA439 | 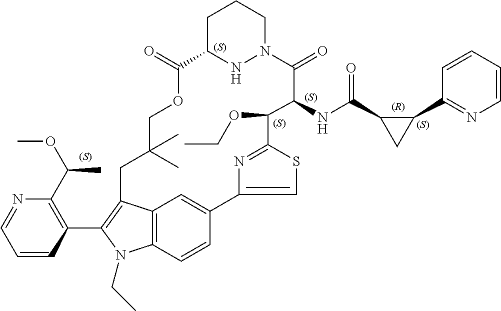 |
| DA440 | 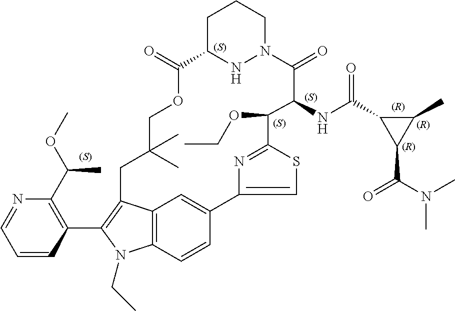 |
| DA441 | 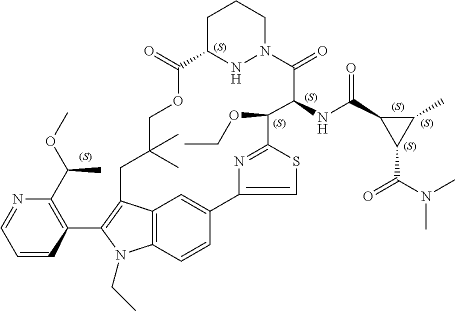 |
| DA442 | 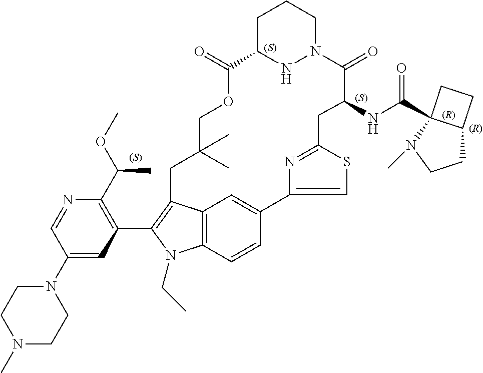 |
| DA443 | 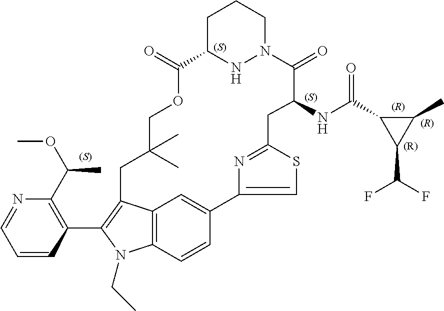 |
| DA444 | 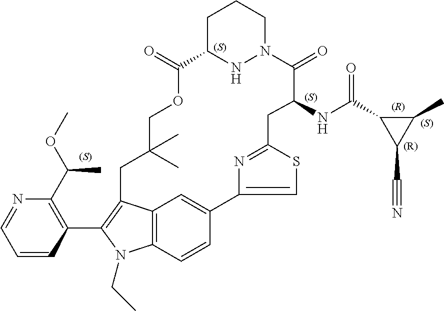 |
| DA445 | 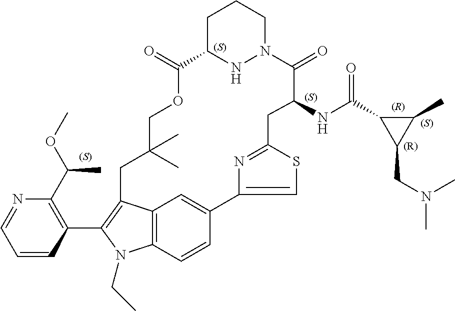 |
| DA446 | 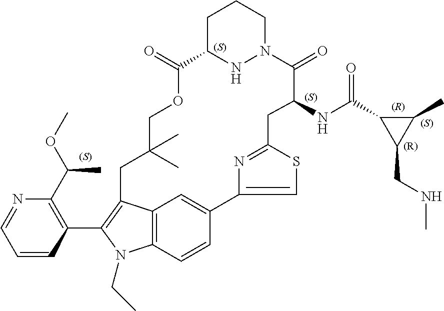 |
| DA447 | 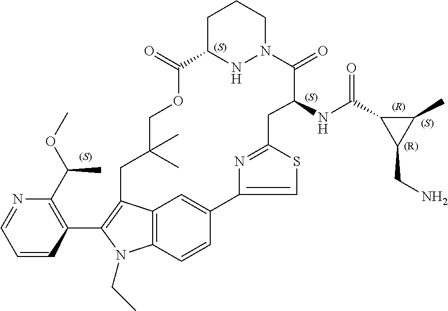 |
| DA448 | 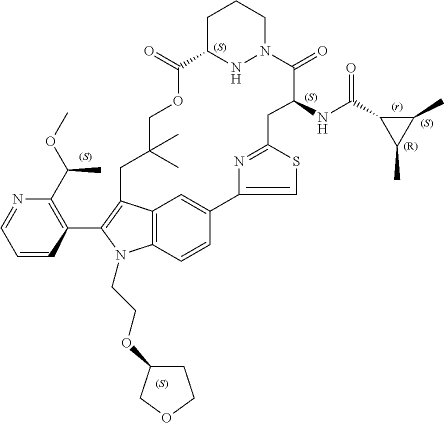 |
| DA449 | 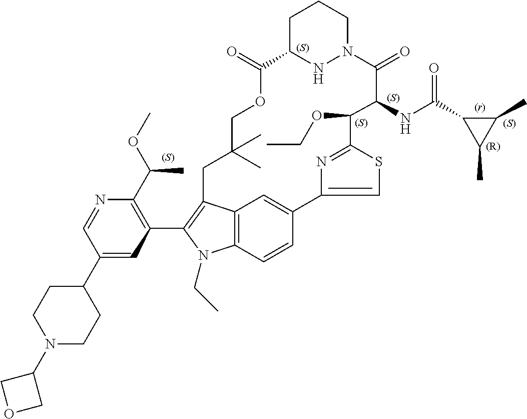 |
| DA450 | 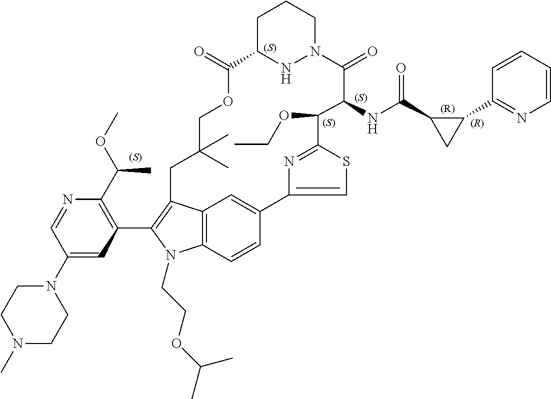 |
| DA451 | 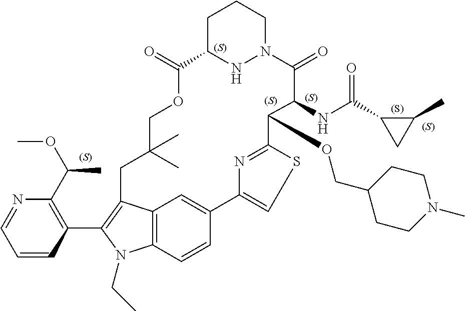 |
| DA452 | 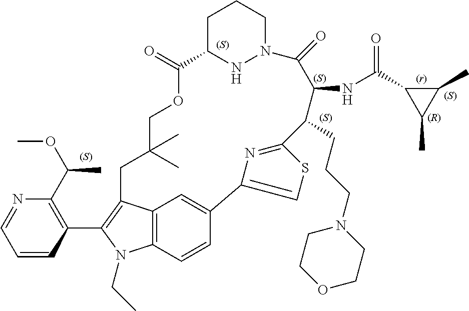 |
| DA453 | 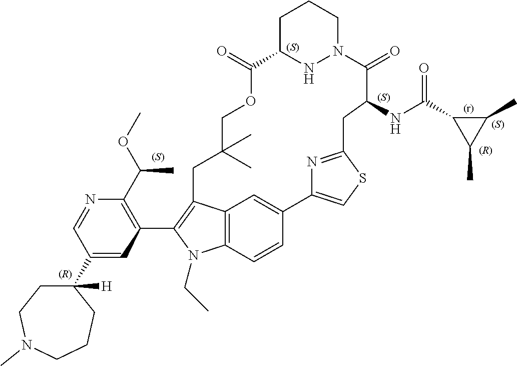 |
| DA454 | 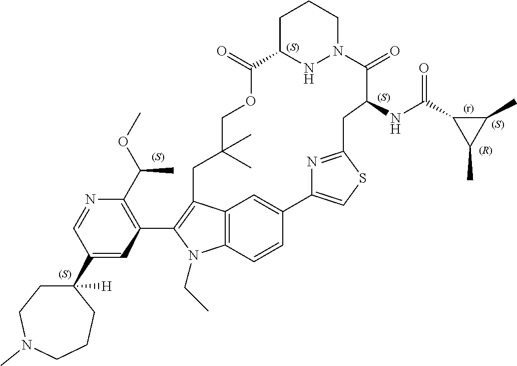 |
| DA455 | 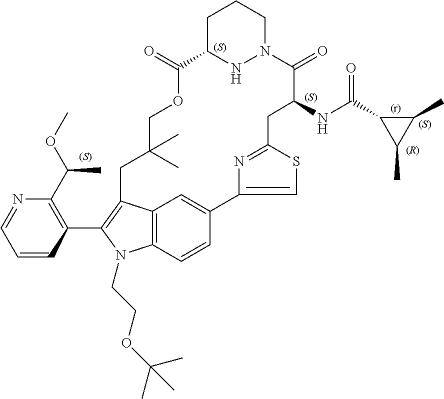 |
| DA456 | 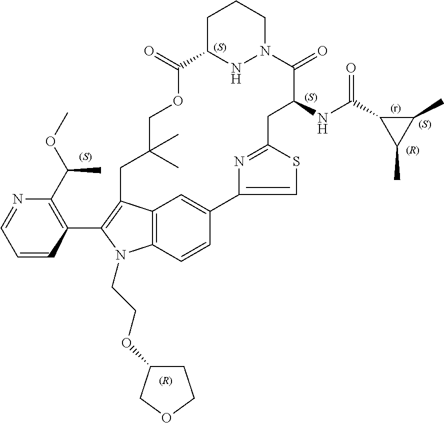 |
| DA457 | 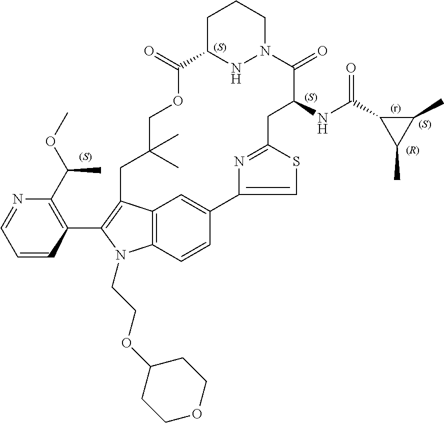 |
| DA458 | 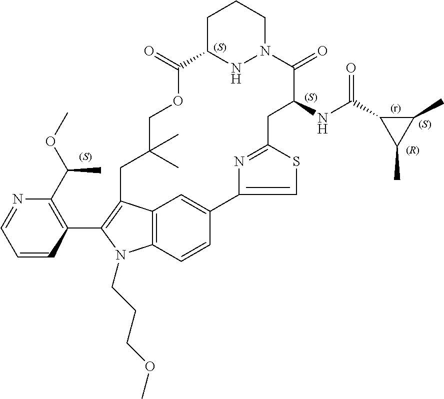 |
| DA459 | 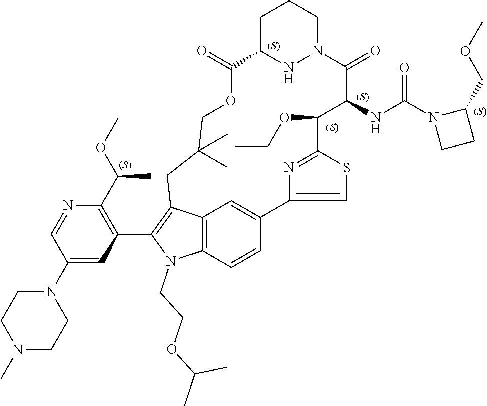 |
| DA460 | 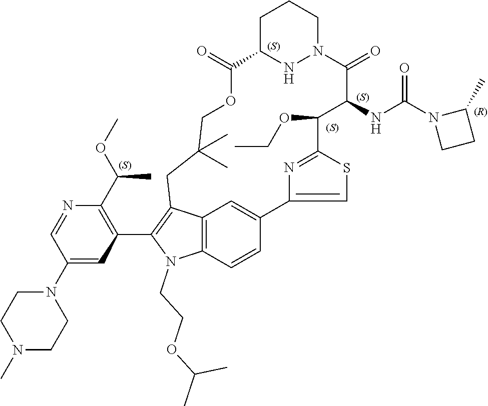 |
| DA461 | 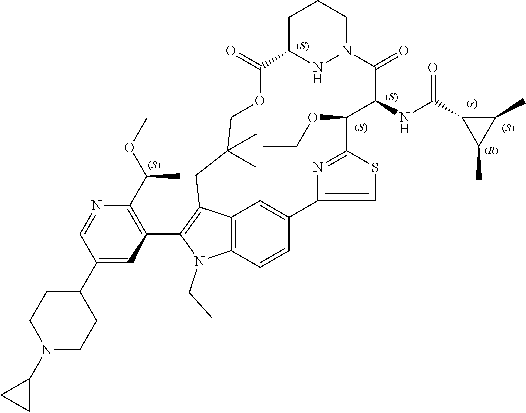 |
| DA462 | 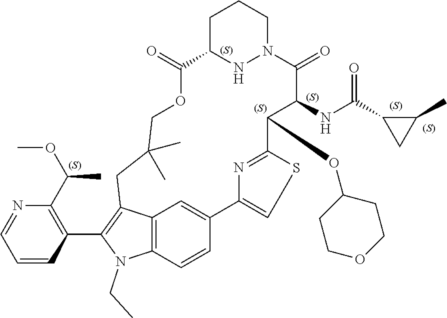 |
| DA463 | 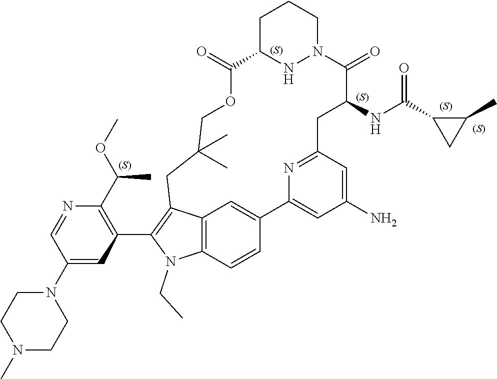 |
| DA464 | 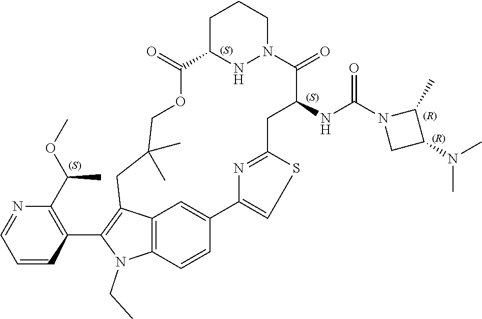 |
| DA465 | 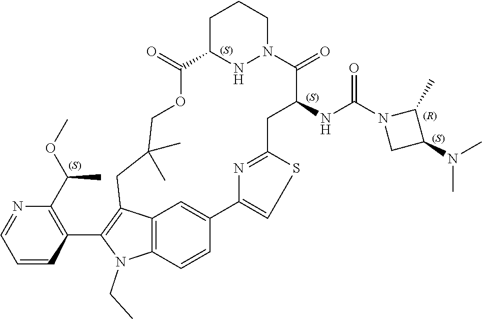 |
| DA466 | 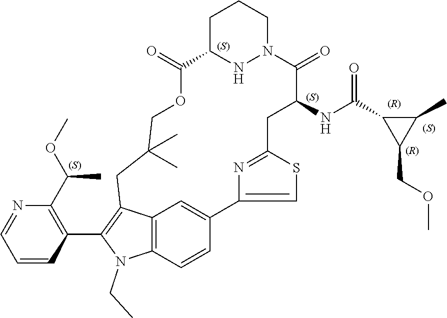 |
| DA467 | 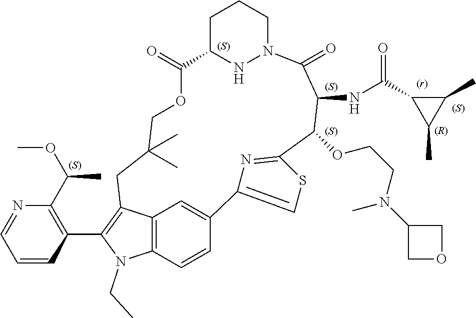 |
| DA468 | 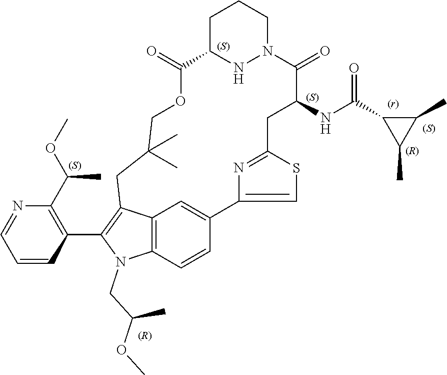 |
| DA469 | 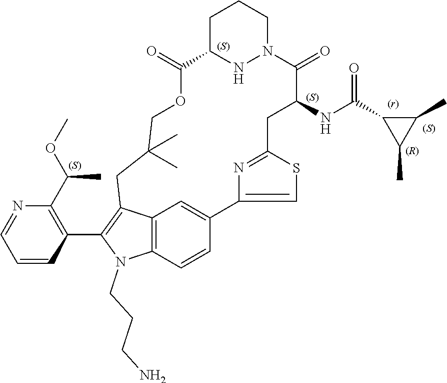 |
| DA470 | 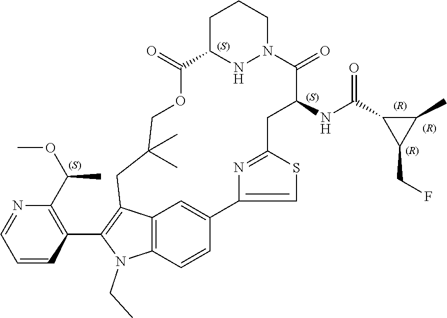 |
| DA471 | 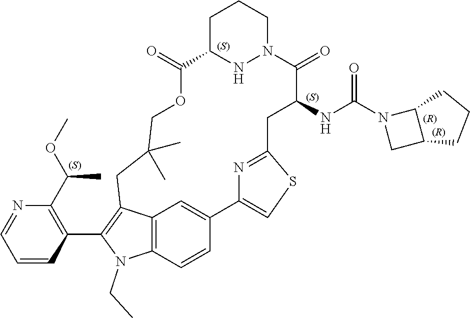 |
| DA472 | 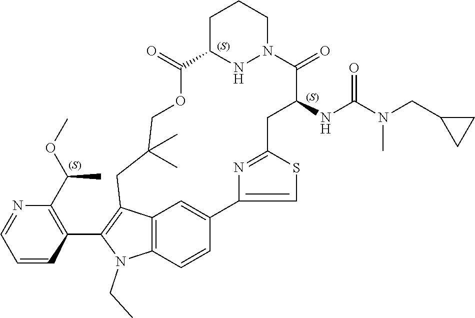 |
| DA473 | 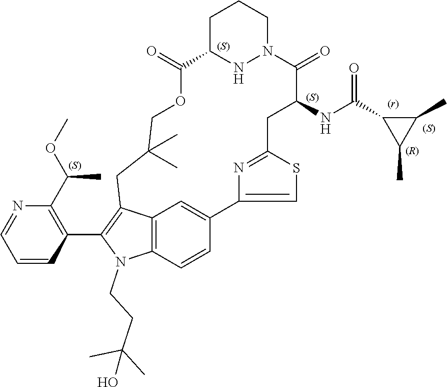 |
| DA474 | 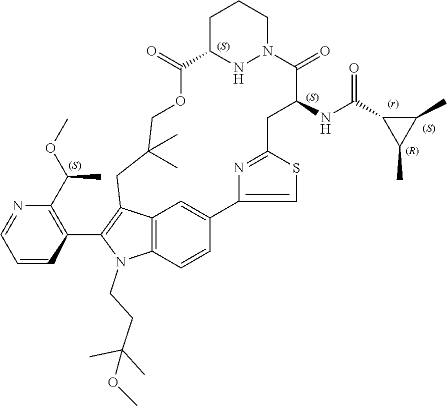 |
| DA475 | 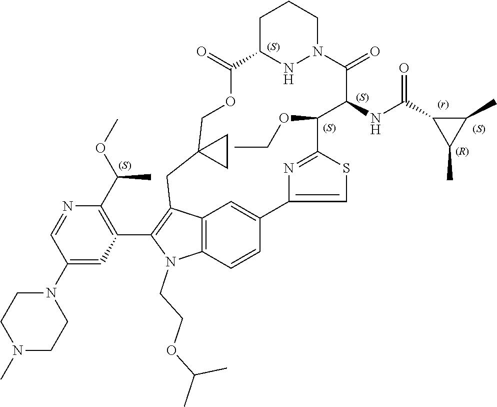 |
| DA476 | 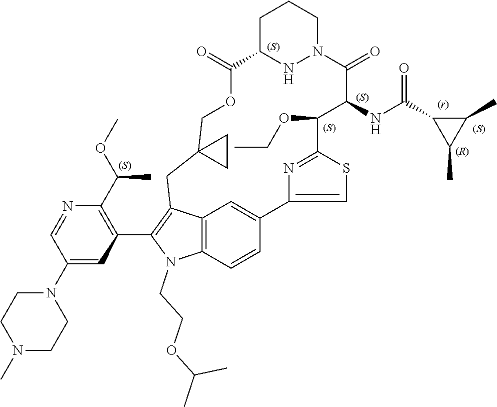 |
| DA477 | 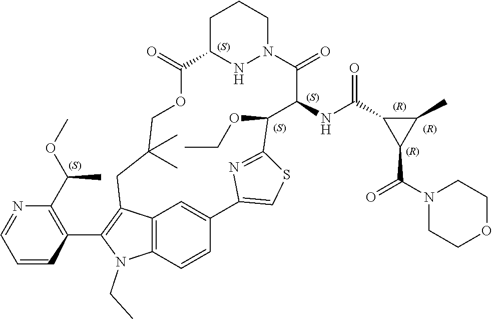 |
| DA478 | 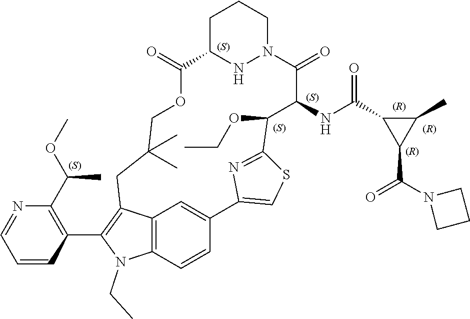 |
| DA479 | 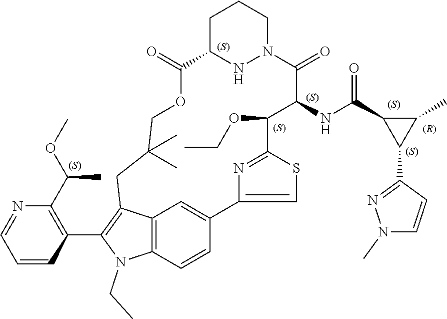 |
| DA480 | 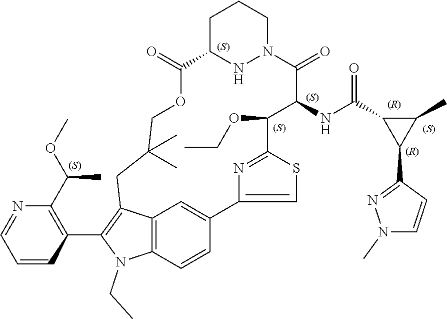 |
| DA481 | 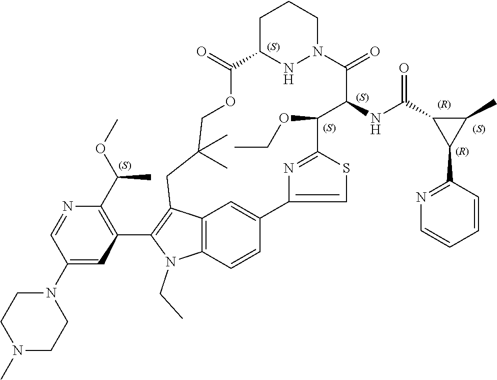 |
| DA482 | 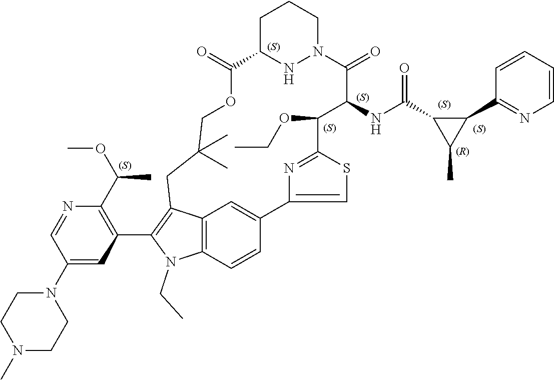 |
| DA483 | 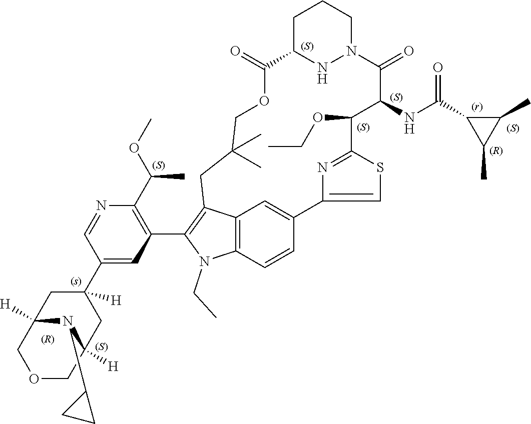 |
| DA484 | 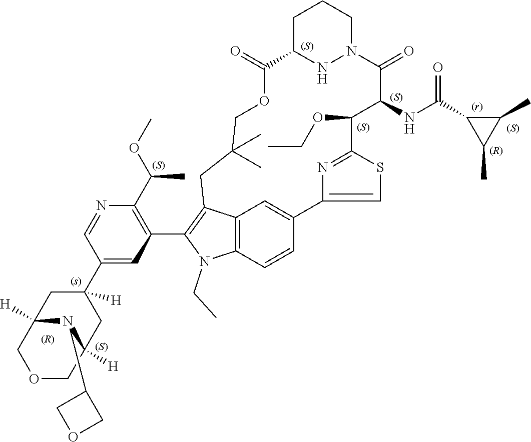 |
| DA485 | 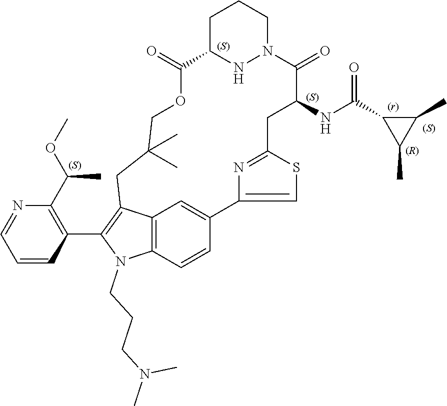 |
| DA486 | 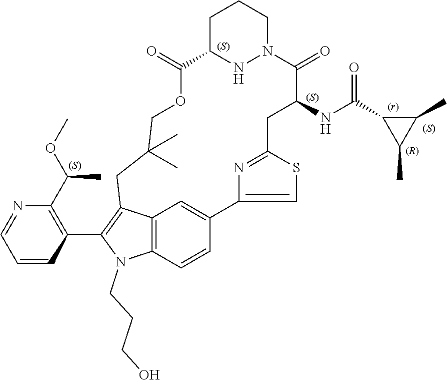 |
| DA487 | 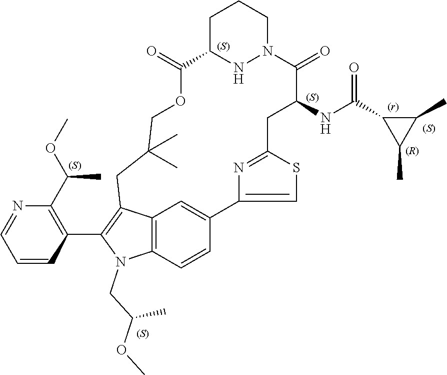 |
| DA488 | 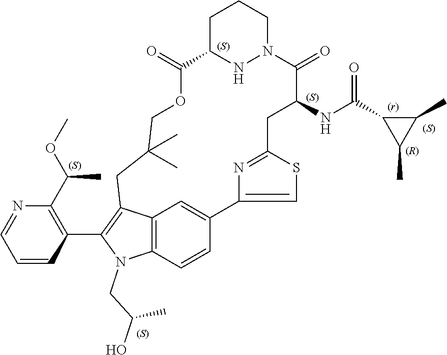 |
| DA489 | 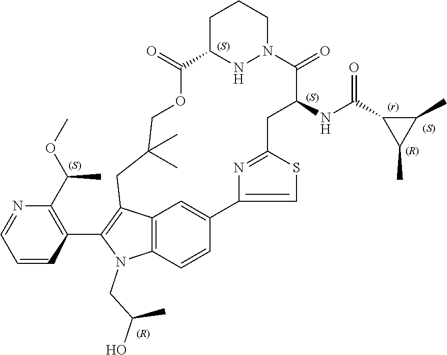 |
| DA490 | 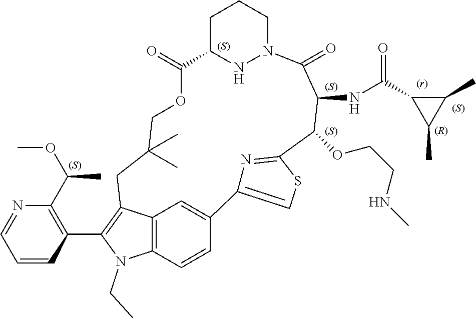 |
| DA491 | 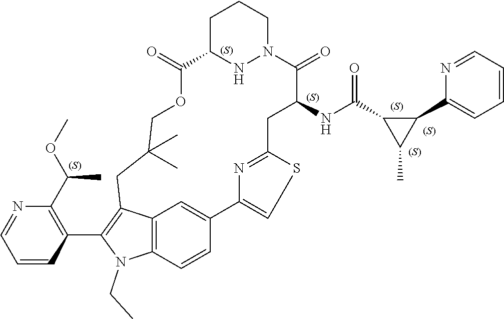 |
| DA492 | 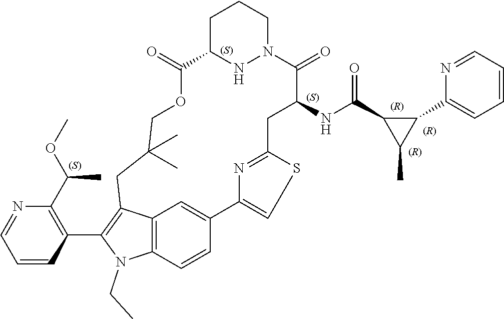 |
| DA493 | 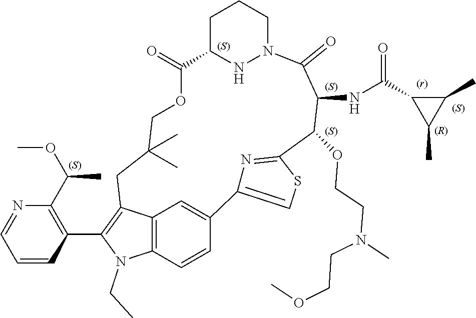 |
| DA494 | 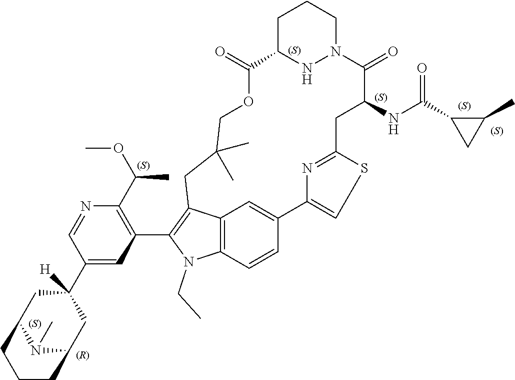 |
| DA495 | 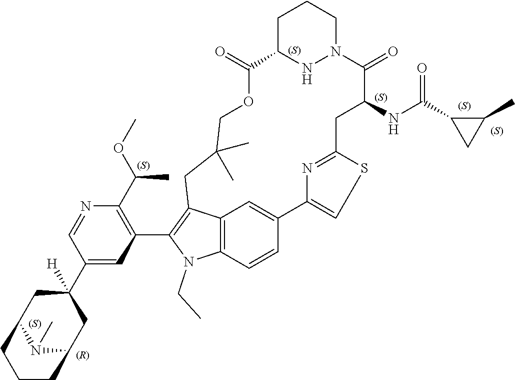 |
| DA496 | 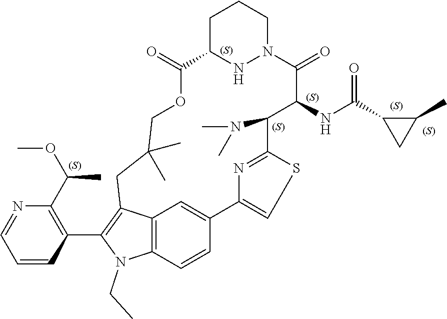 |
| DA497 | 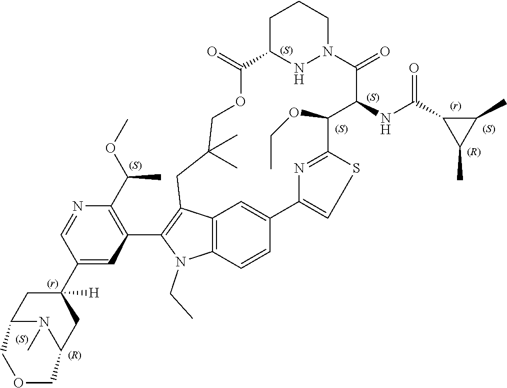 |
| DA498 | 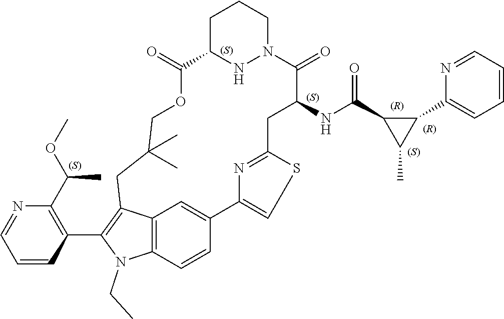 |
| DA499 | 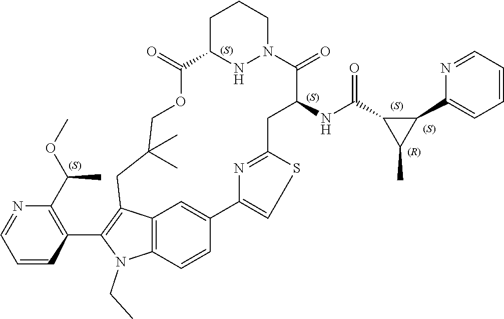 |
| DA500 | 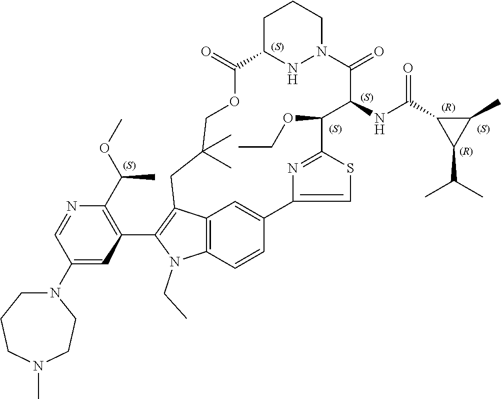 |
| DA501 | 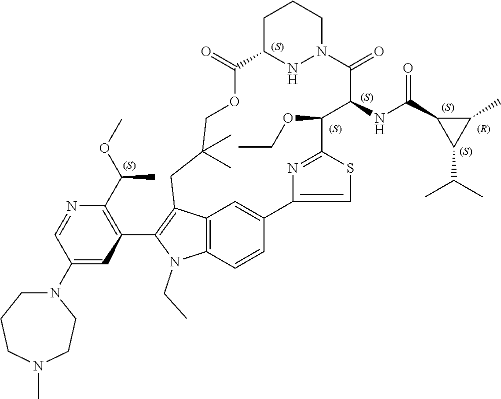 |
| DA502 | 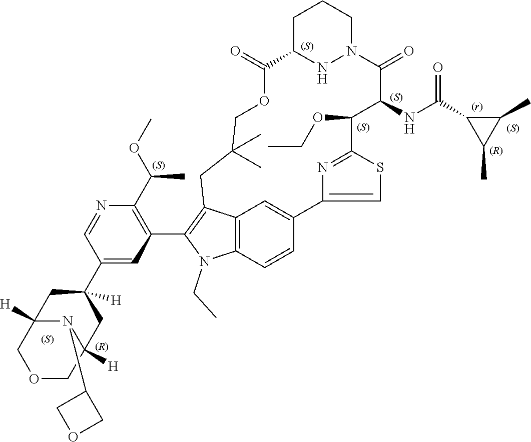 |
| DA503 | 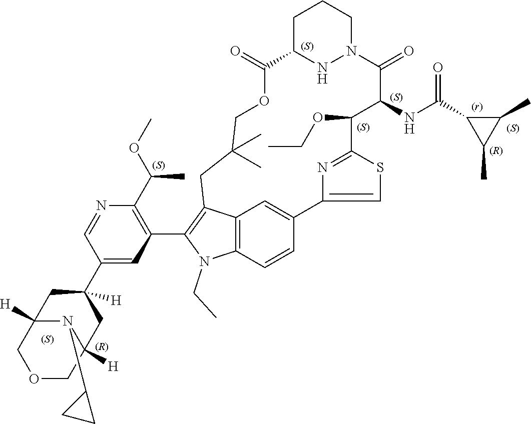 |
| DA504 | 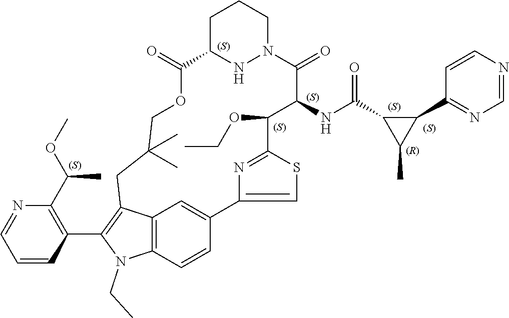 |
| DA505 | 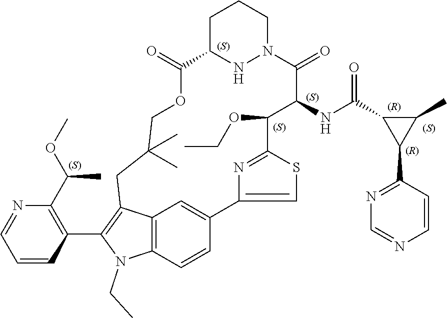 |
| DA506 | 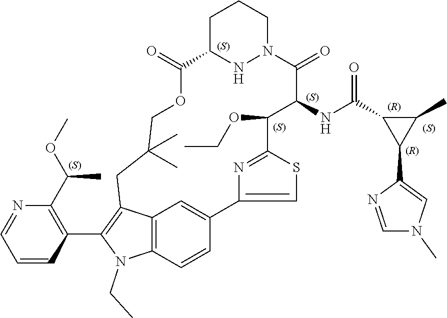 |
| DA507 | 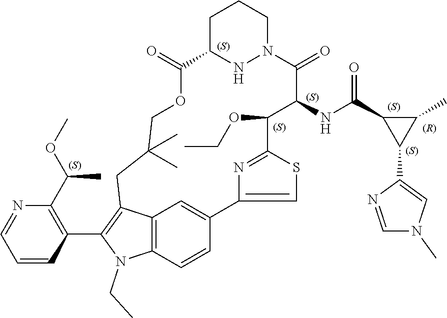 |
| DA508 | 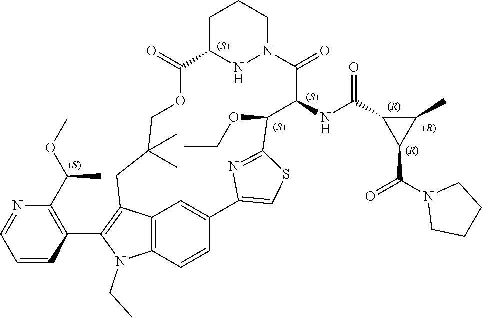 |
| DA509 | 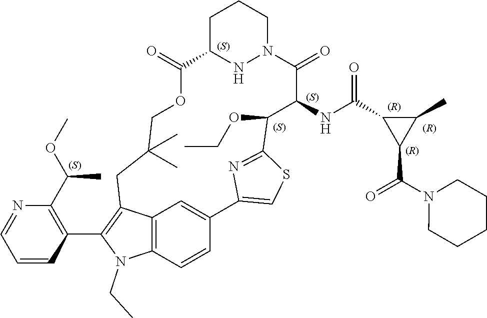 |
| DA510 | 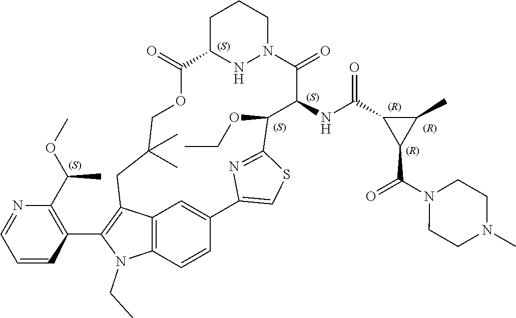 |
| DA511 | 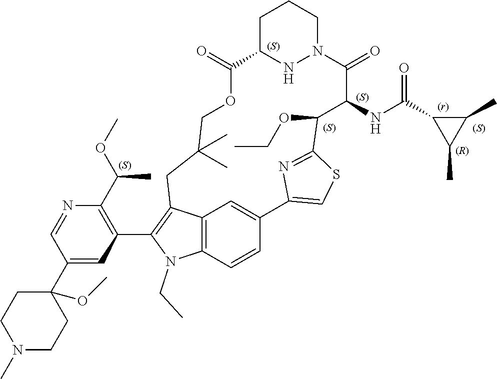 |
| DA512 | 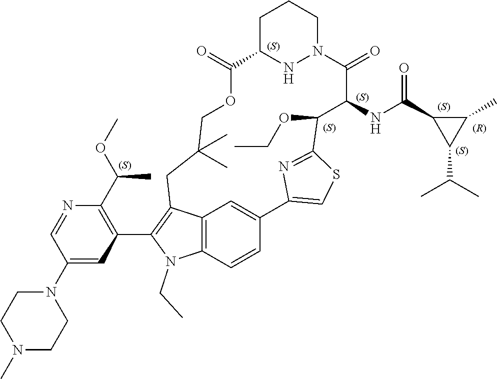 |
| DA513 | 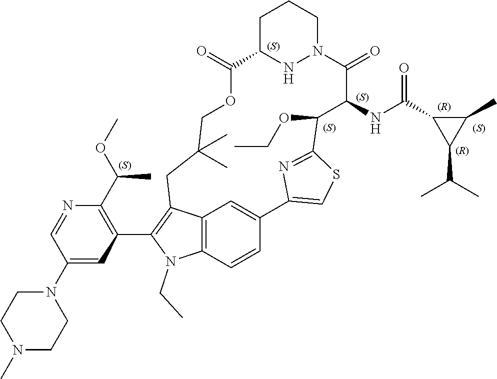 |
| DA514 | 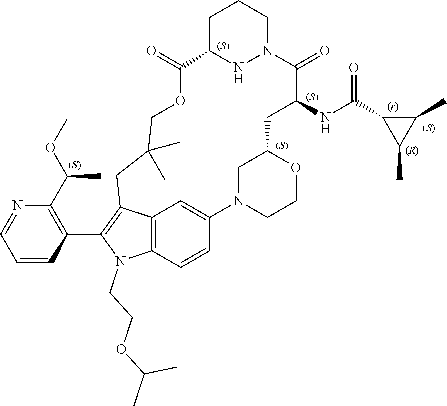 |
| DA515 | 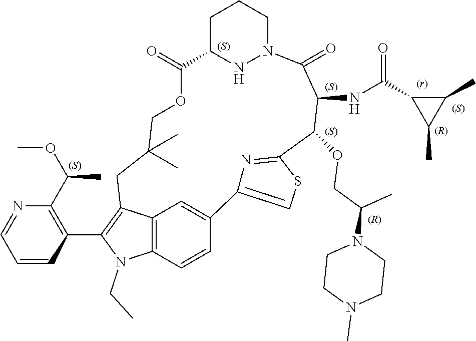 |
| DA516 | 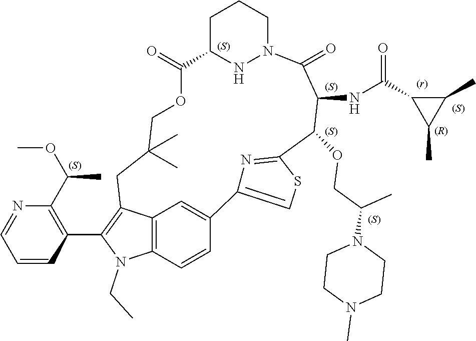 |
| DA517 | 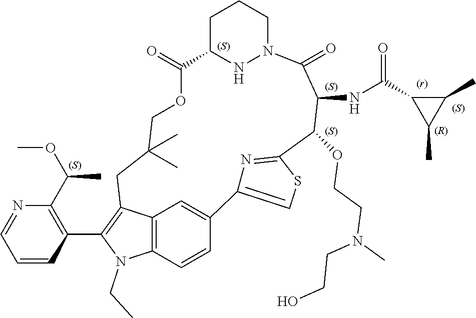 |
| DA518 | 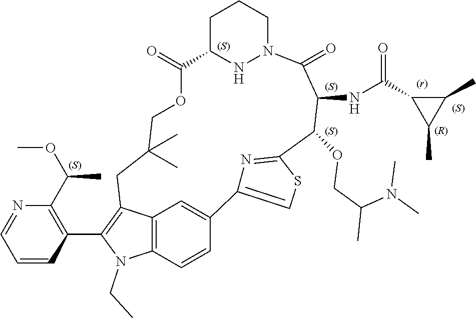 |
| DA519 | 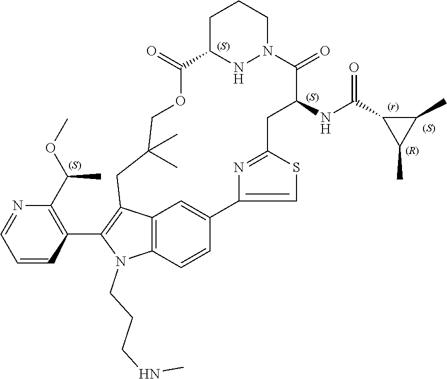 |
| DA520 | 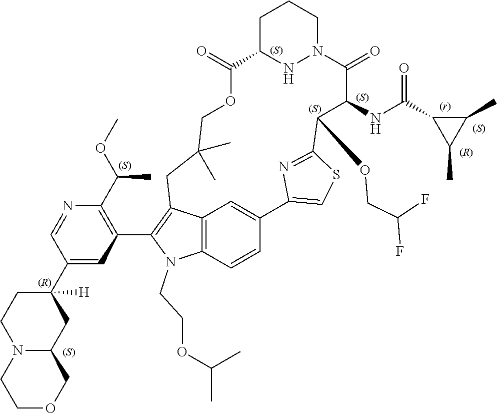 |
| DA521 | 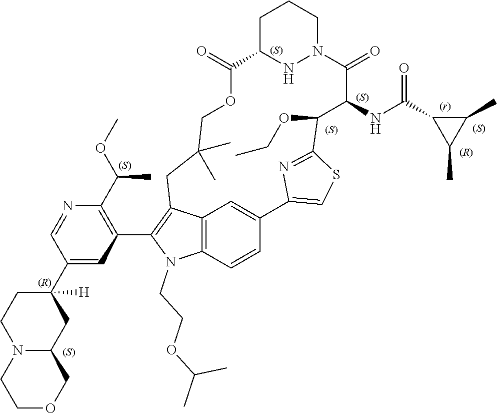 |
| DA522 | 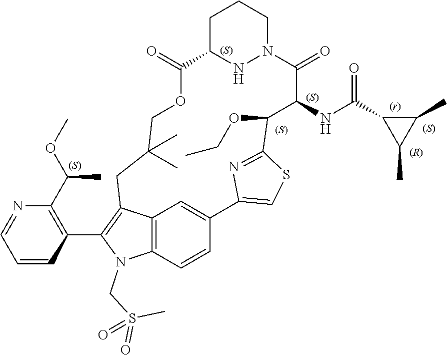 |
| DA523 | 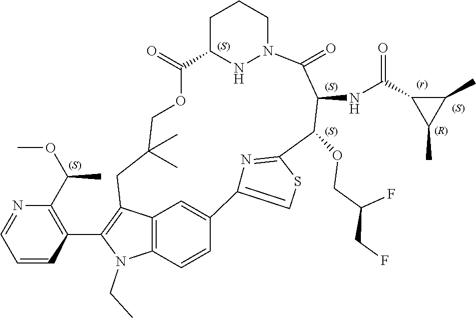 |
| DA524 | 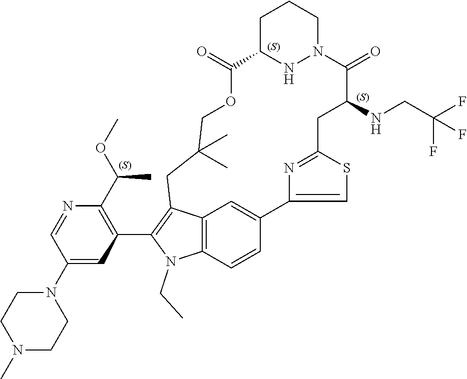 |
| DA525 | 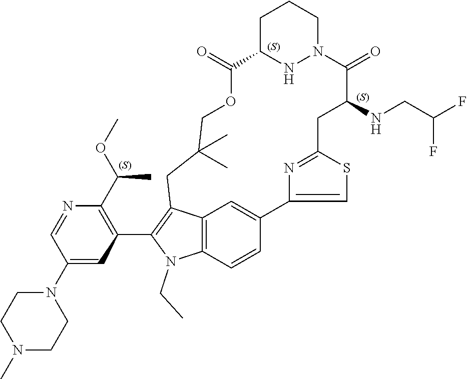 |
| DA526 | 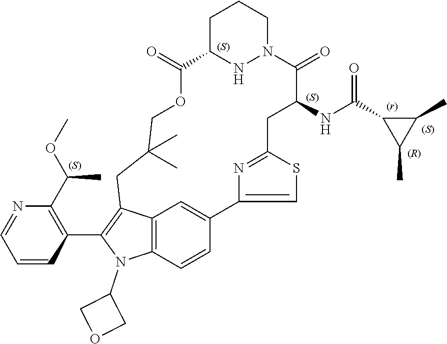 |
| DA527 | 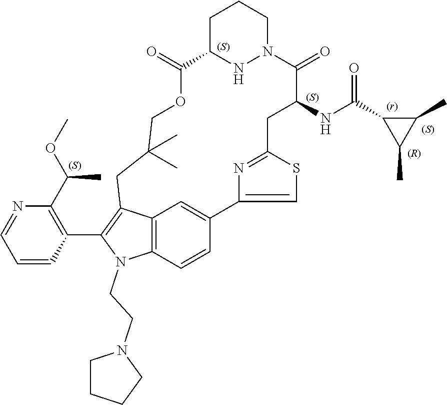 |
| DA528 | 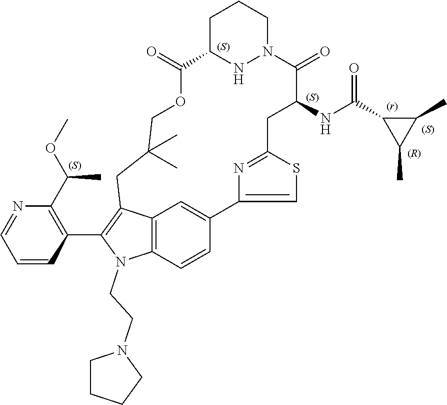 |
| DA529 | 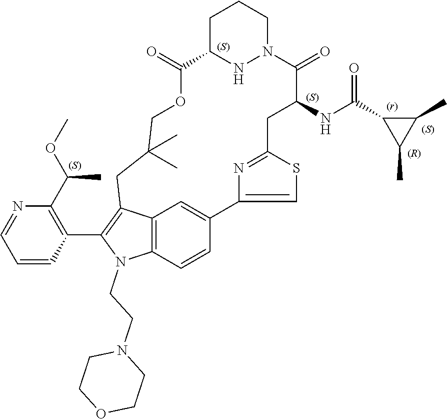 |
| DA530 | 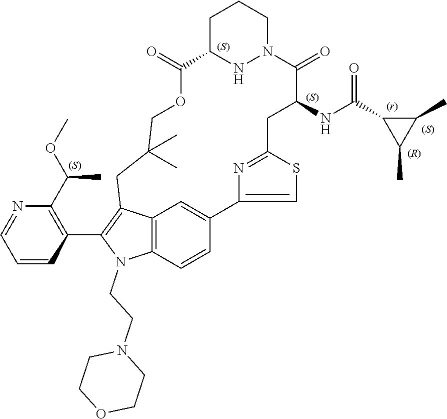 |
| DA531 | 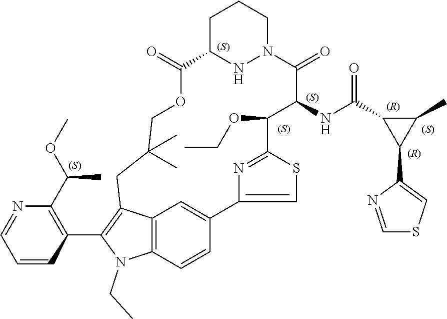 |
| DA532 | 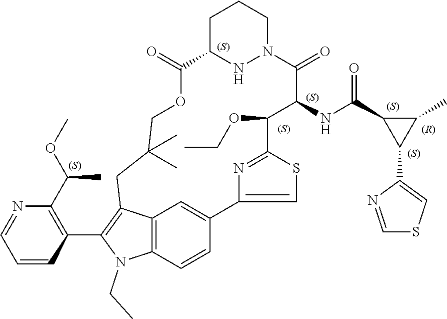 |
| DA533 | 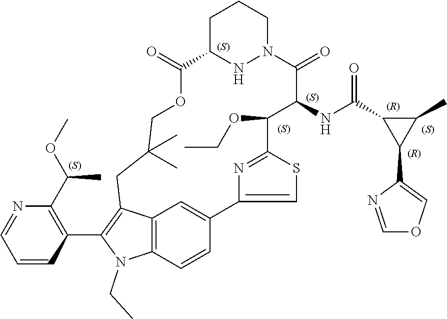 |
| DA534 | 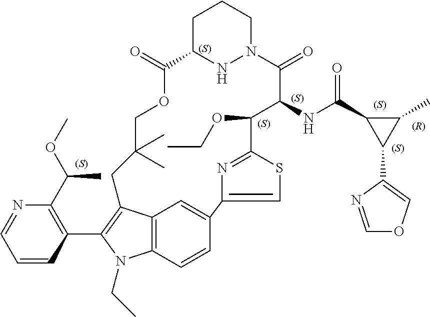 |
| DA535 | 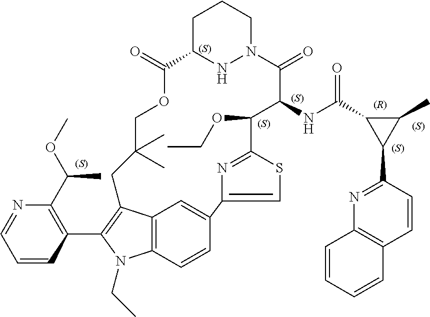 |
| DA536 | 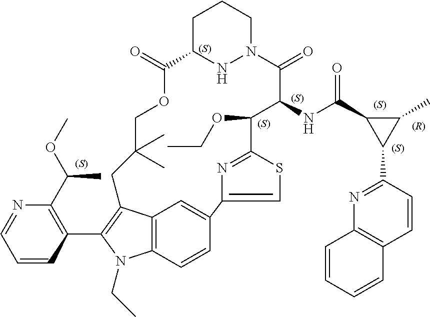 |
| DA537 | 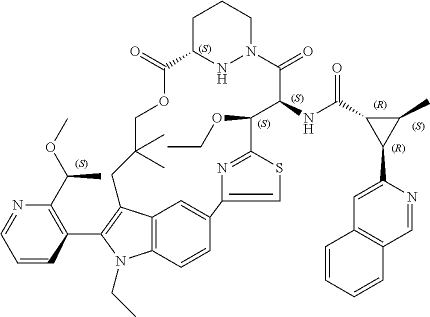 |
| DA538 | 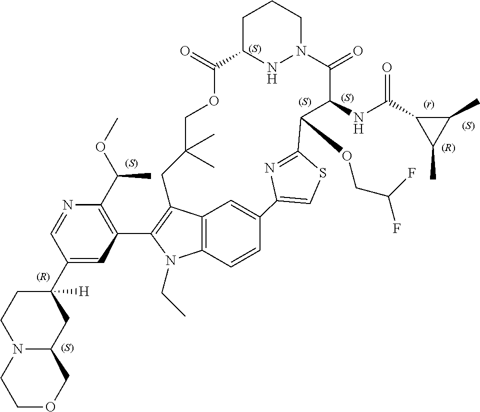 |
| DA539 | 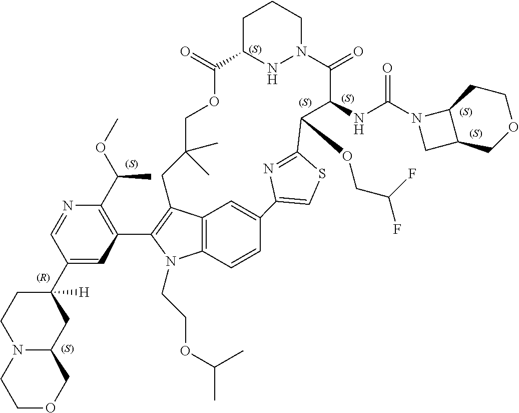 |
| DA540 | 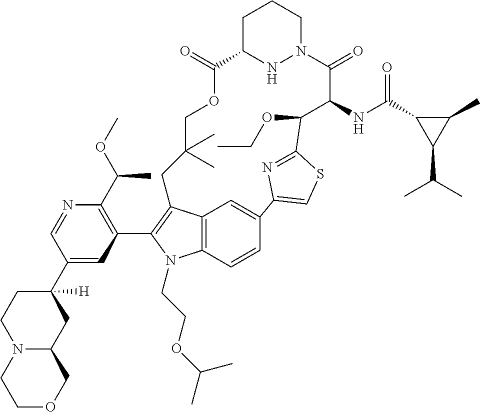 |
| DA541 | 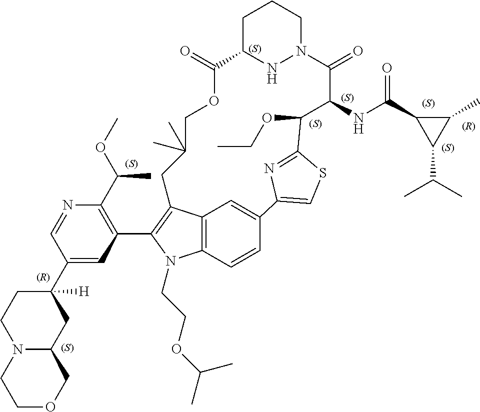 |
| DA542 | 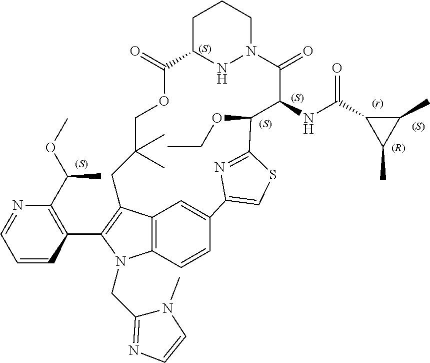 |
| DA543 | 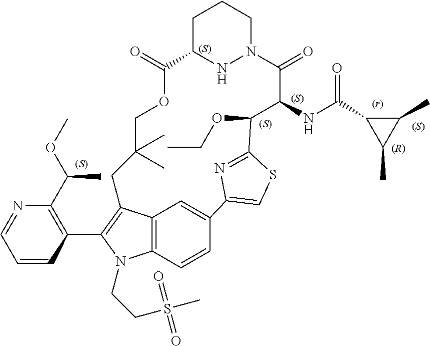 |
| DA544 | 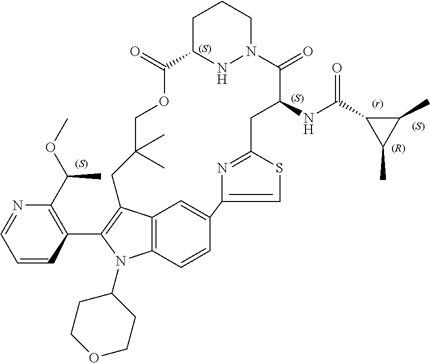 |
| DA545 | 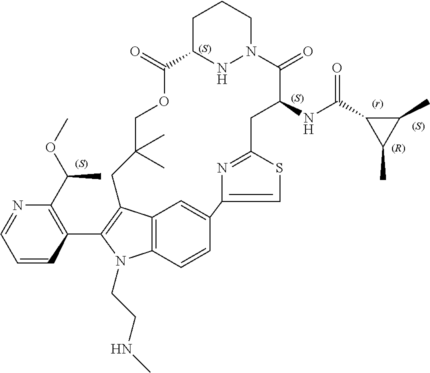 |
| DA546 | 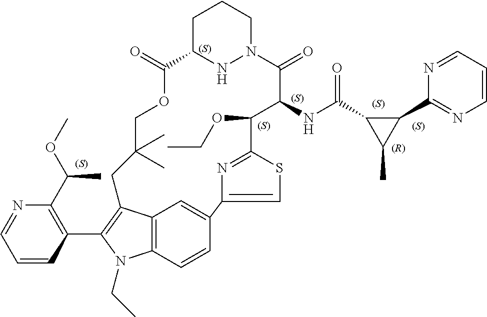 |
| DA547 | 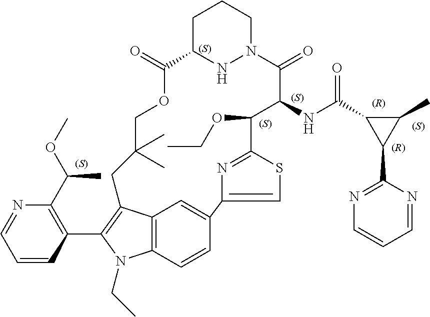 |
| DA548 | 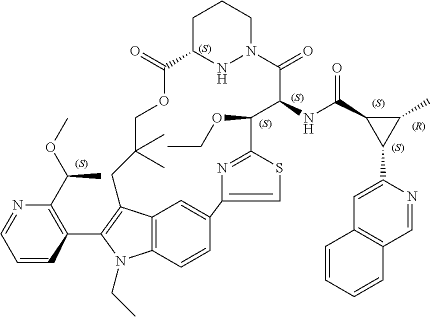 |
| DA549 | 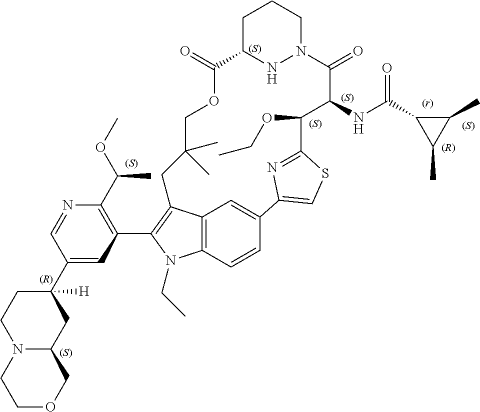 |
| DA550 | 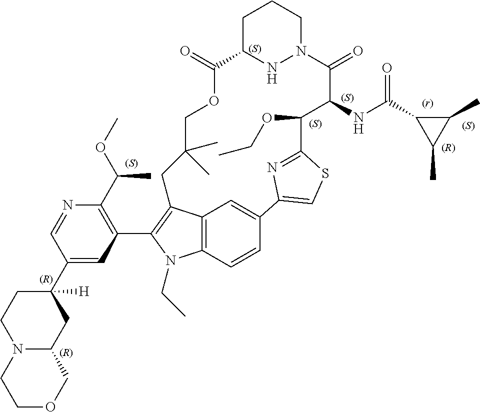 |
| DA551 | 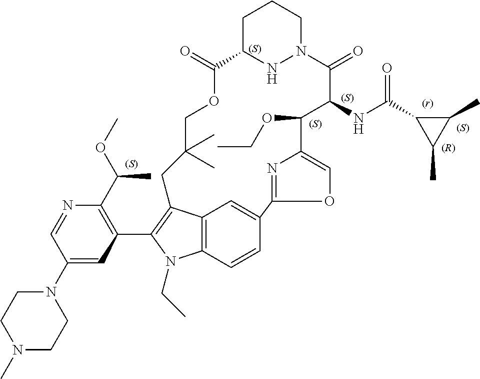 |
| DA552 | 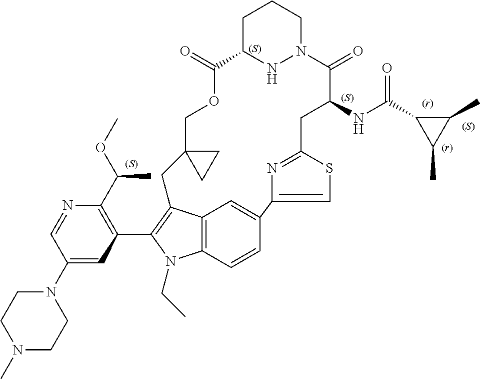 |
| DA553 | 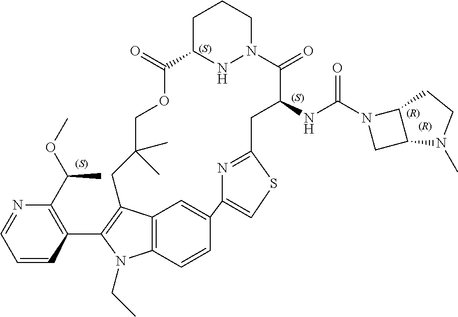 |
| DA554 | 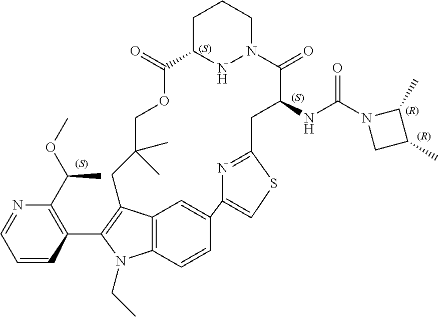 |
| DA555 | 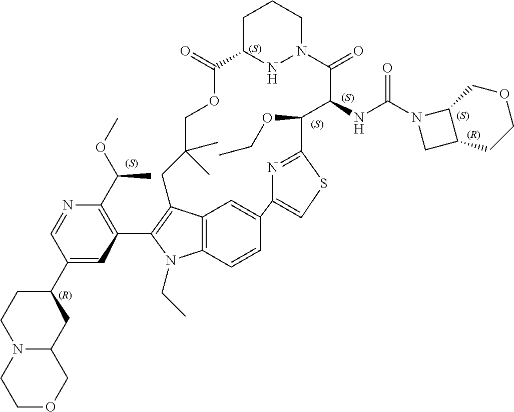 |
| DA556 | 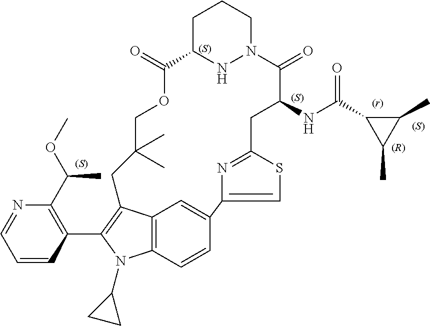 |
| DA557 | 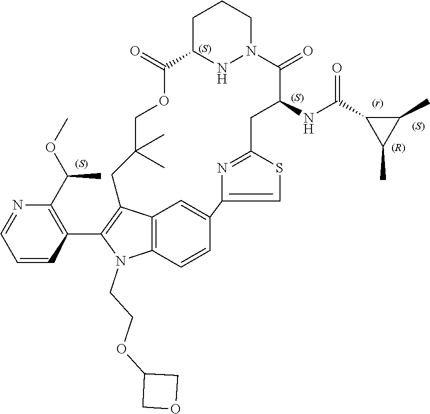 |
| DA558 | 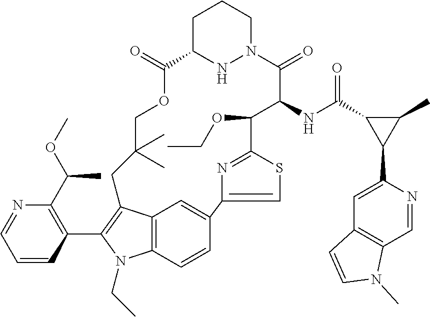 |
| DA559 | 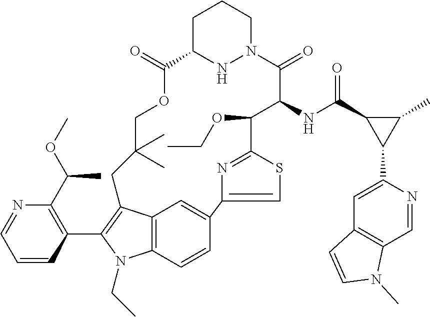 |
| DA560 | 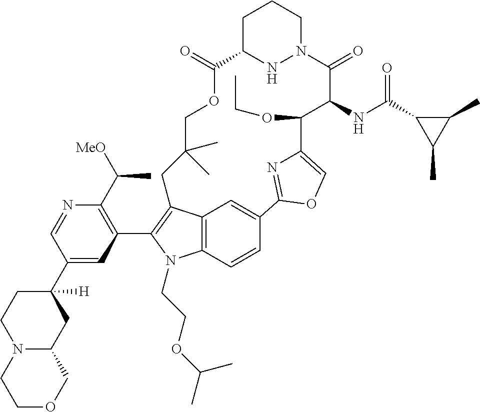 |
| DA561 | 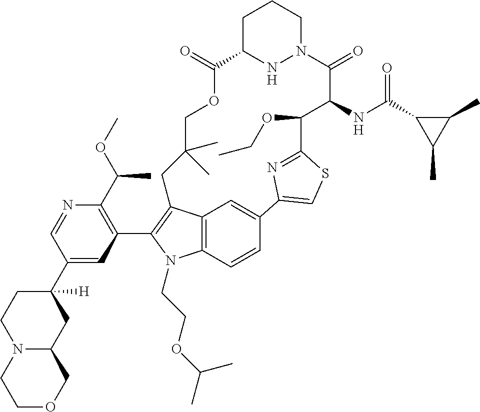 |
| DA562 | 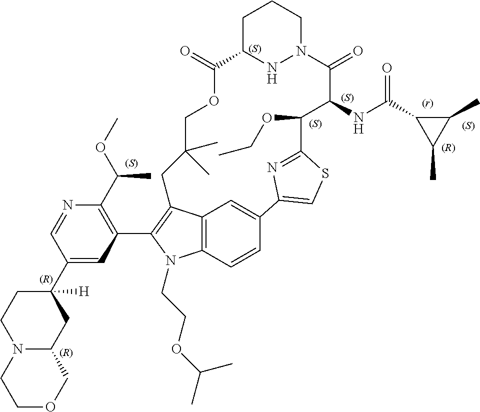 |
| DA563 | 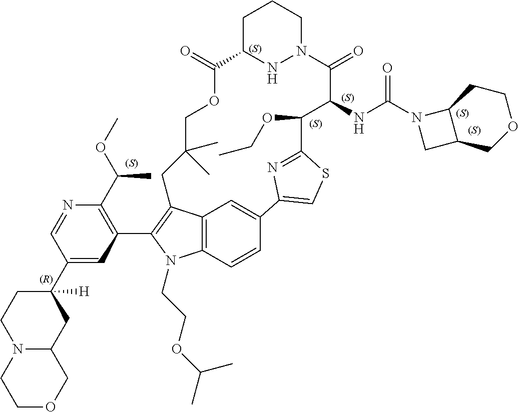 |
| DA564 | 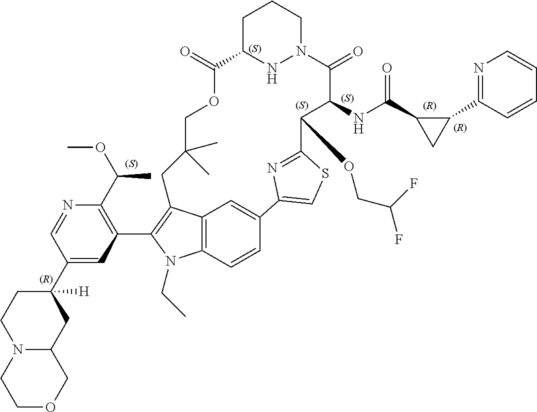 |
| DA565 | 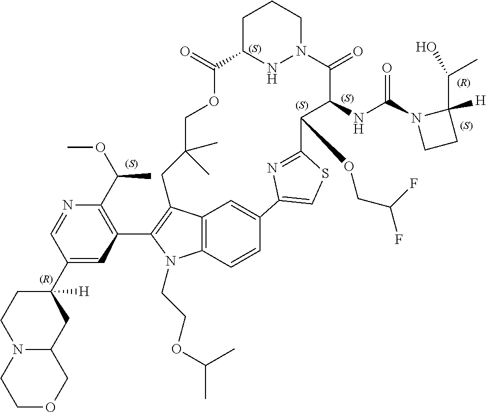 |
| DA566 | 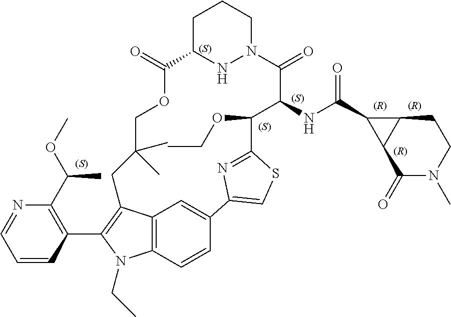 |
| DA567 | 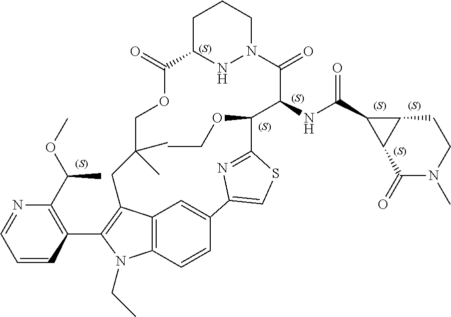 |
| DA568 | 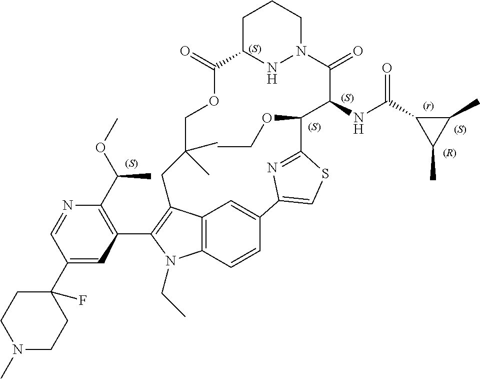 |
| DA569 | 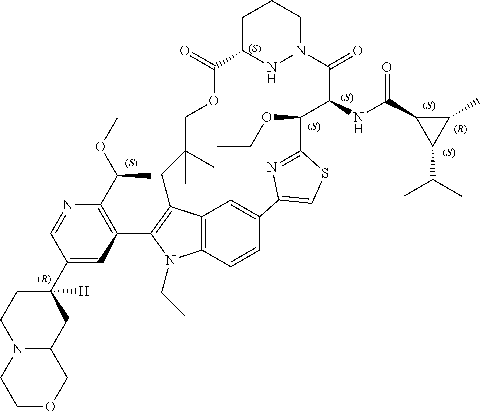 |
| DA570 | 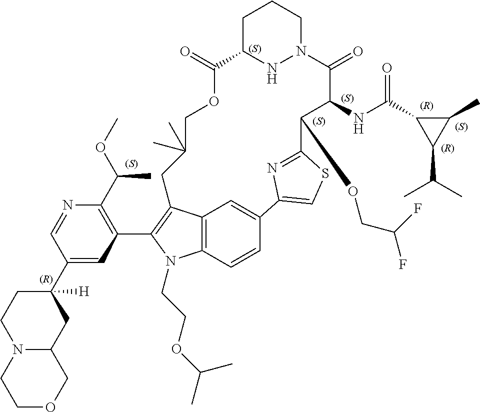 |
| DA571 | 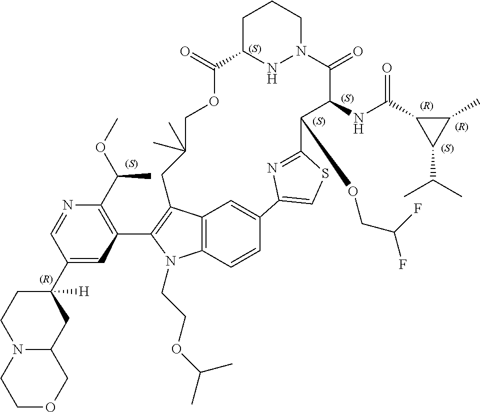 |
| DA572 | 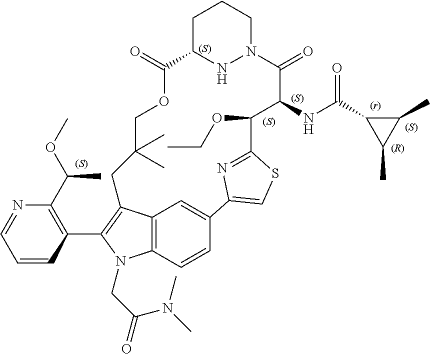 |
| DA573 | 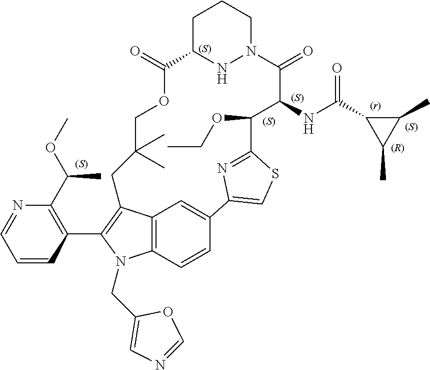 |
| DA574 | 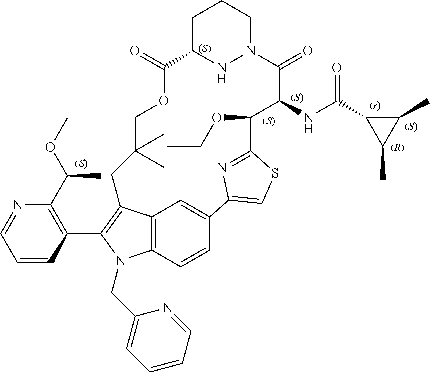 |
| DA575 | 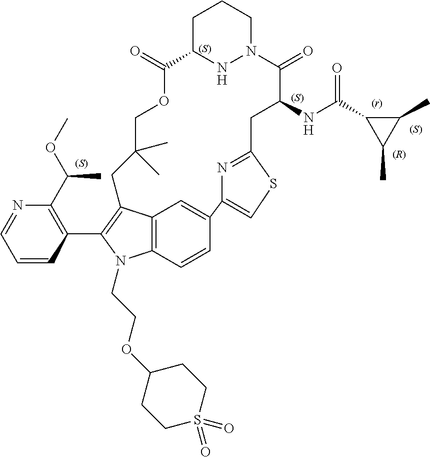 |
| DA576 | 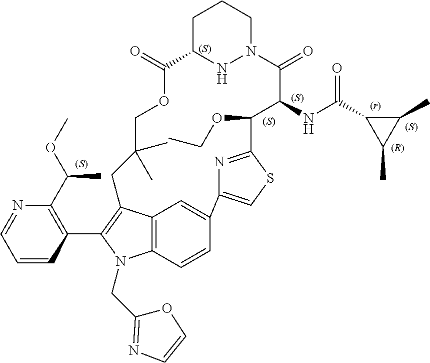 |
| DA577 | 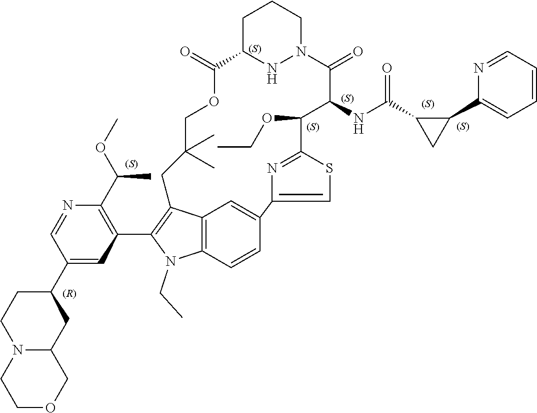 |
| DA578 | 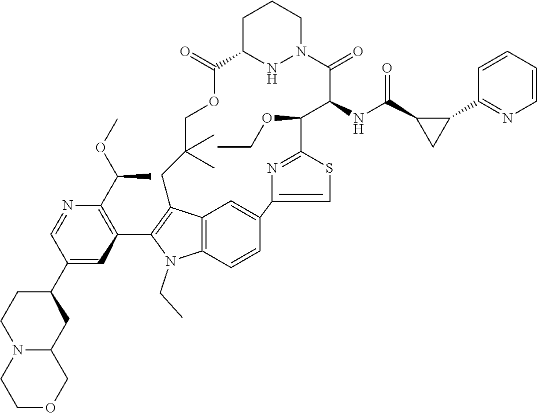 |
| DA579 | 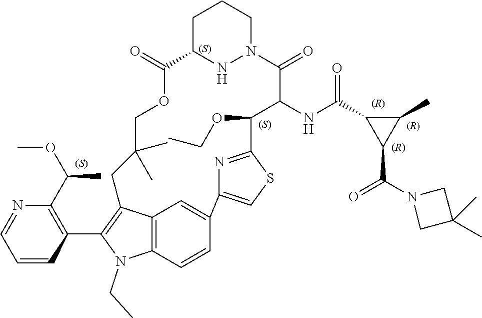 |
| DA580 | 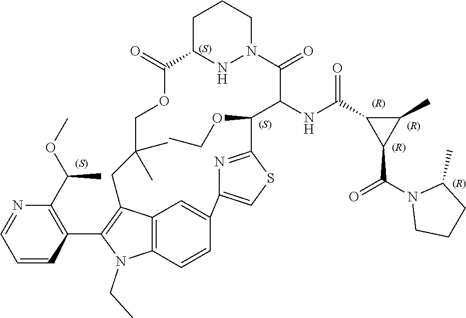 |
| DA581 | 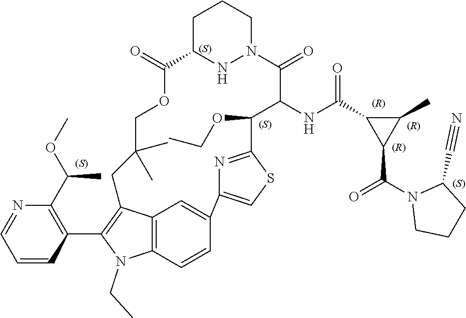 |
| DA582 | 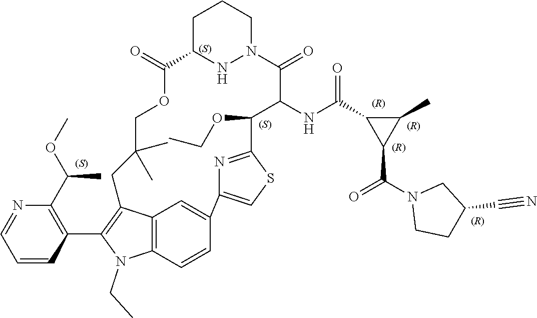 |
| DA583 | 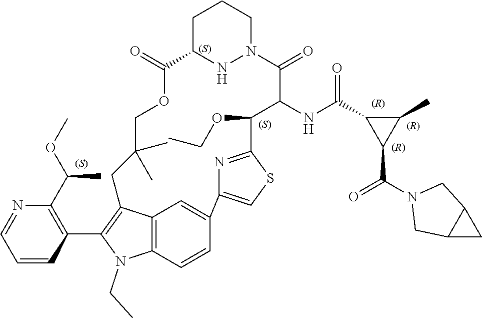 |
| DA584 | 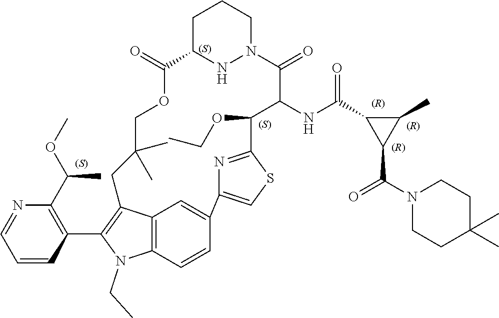 |
| DA585 | 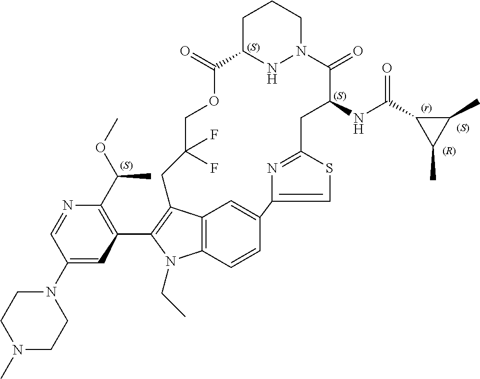 |
| DA586 | 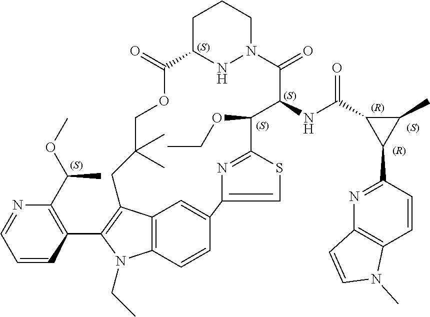 |
| DA587 | 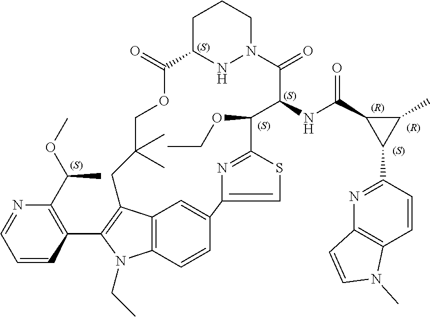 |
| DA588 | 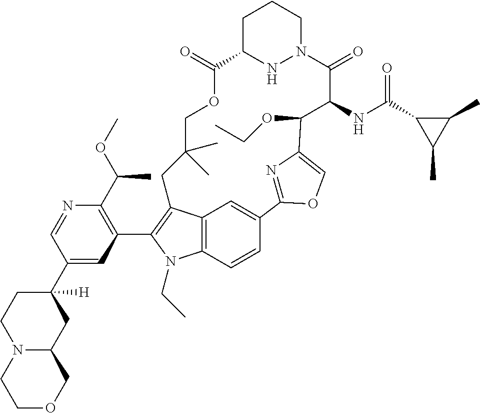 |
| DA589 | 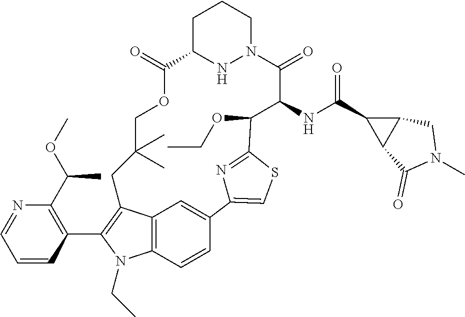 |
| DA590 | 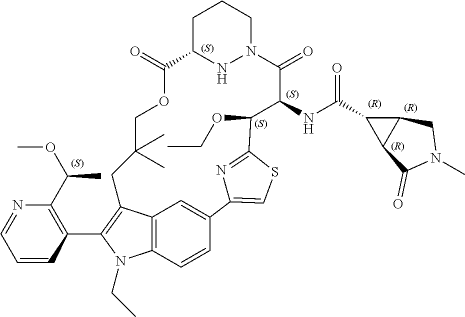 |
| DA591 | 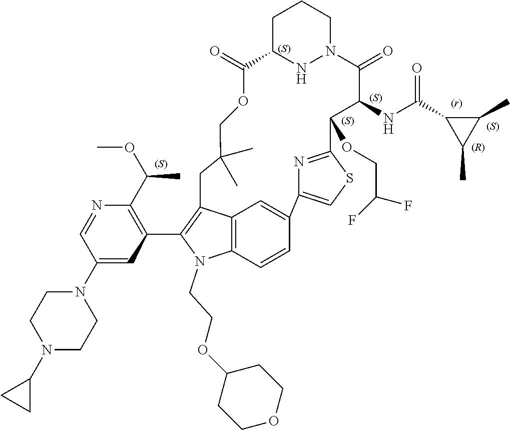 |
| DA592 | 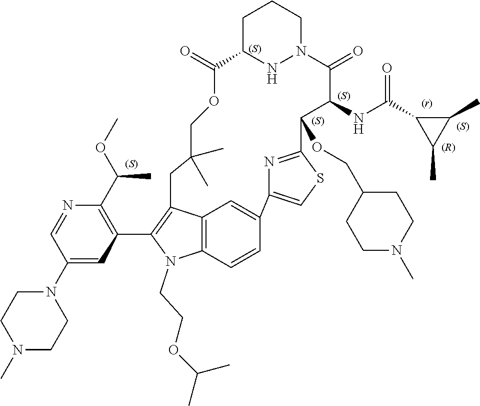 |
| DA593 | 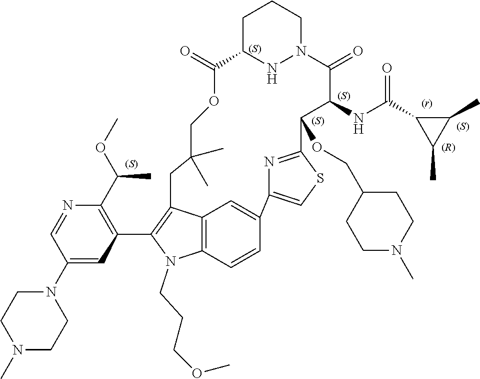 |
| DA594 | 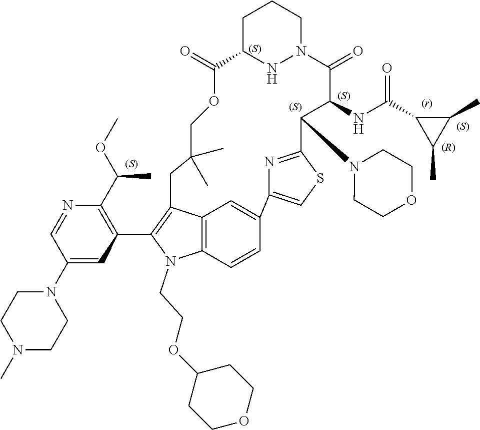 |
| DA595 | 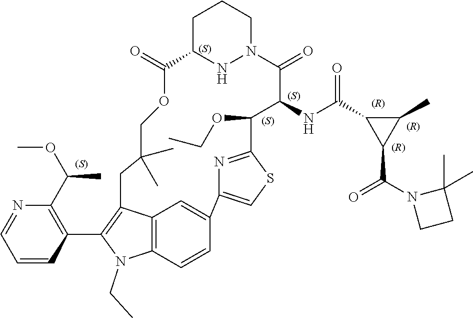 |
| DA596 | 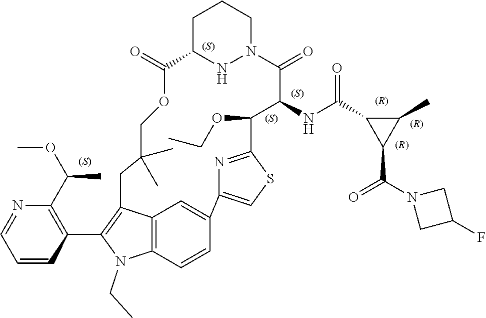 |
| DA597 | 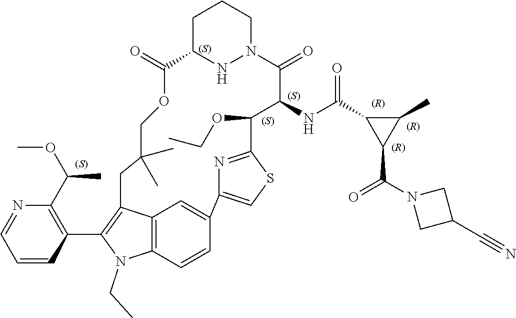 |
| DA598 | 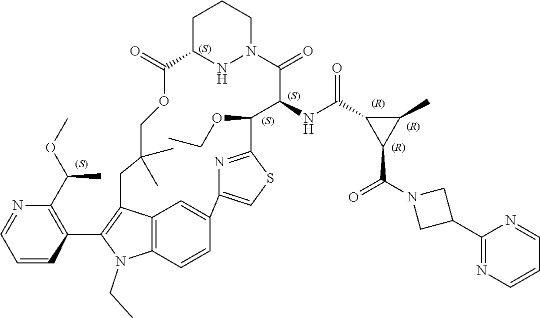 |
| DA599 | 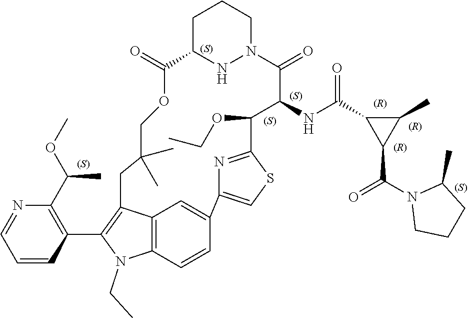 |
| DA600 | 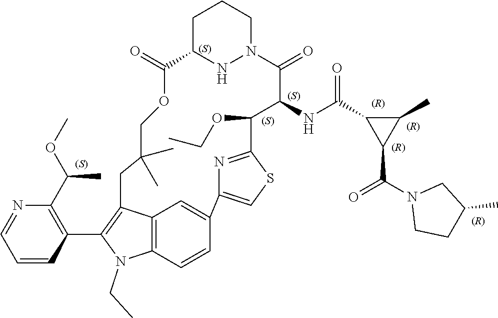 |
| DA601 | 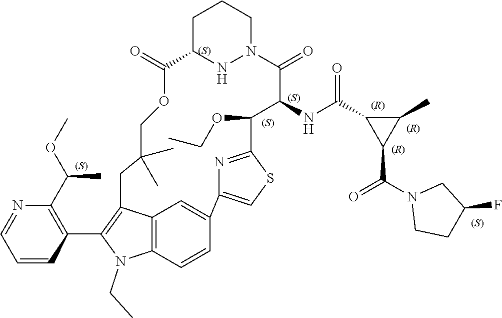 |
| DA602 | 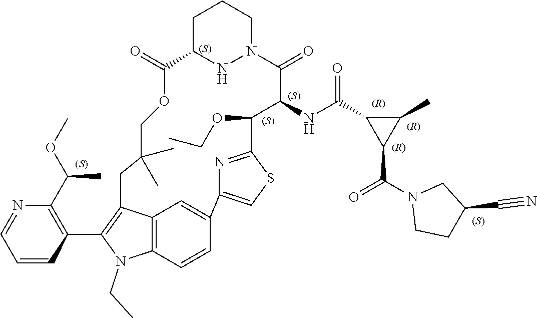 |
| DA603 | 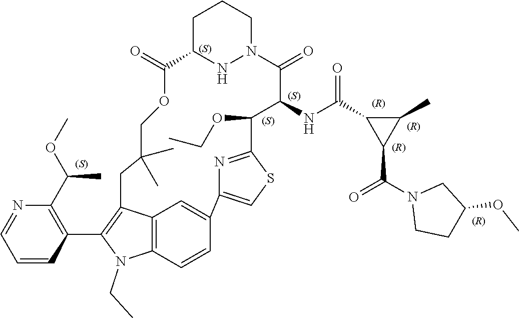 |
| DA604 | 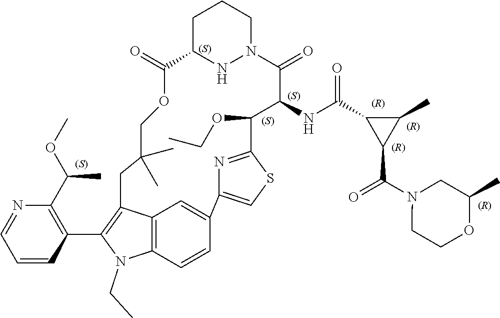 |
| DA605 | 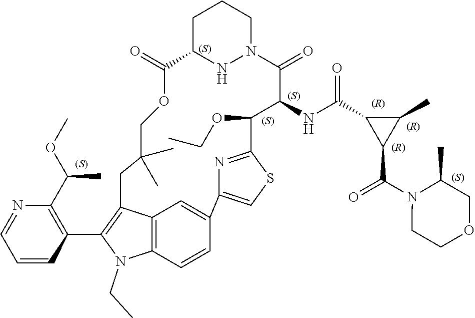 |
| DA606 | 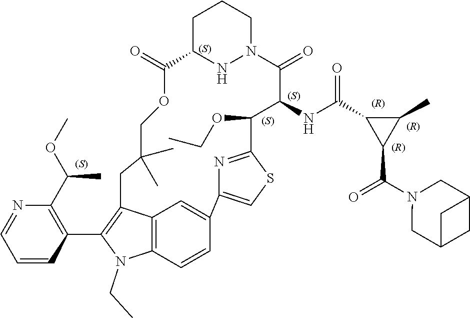 |
| DA607 | 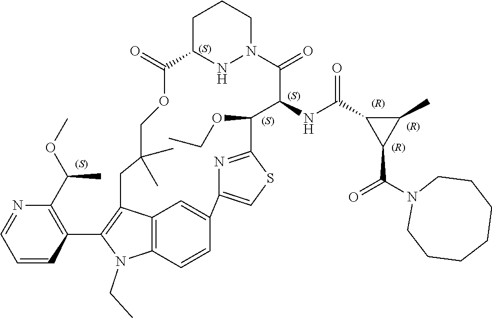 |
| DA608 | 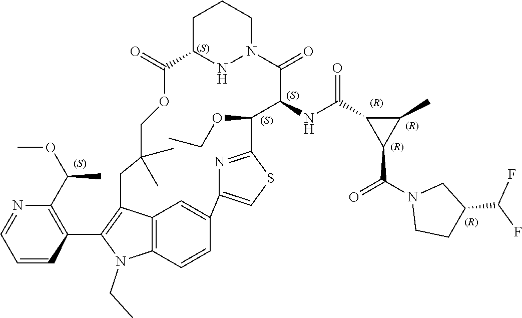 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the
absolute stereochemistry of such diastereomer may not be known.

In some embodiments, the RAS(ON) inhibitor is a compound selected from Table D2, or a pharmaceutically acceptable salt or stereoisomer thereof. In some embodiments, the RAS(ON) inhibitor is a compound selected from Table D2, or a pharmaceutically acceptable salt or atropisomer thereof

In some embodiments, the RAS(ON) inhibitor is not a compound selected from Table D2. In some embodiments, the RAS(ON) inhibitor is not a compound selected from Table D2, or a pharmaceutically acceptable salt or stereoisomer thereof. In some embodiments, the RAS(ON) inhibitor is not a compound selected from Table D2, or a pharmaceutically acceptable salt or atropisomer thereof.

TABLE D2
Certain Compounds

| Ex. # | Structure |
|---|---|
| DB1 | 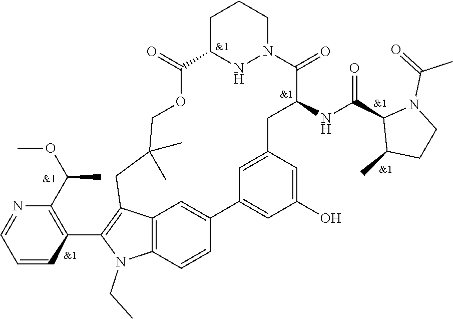 |
| DB2 | 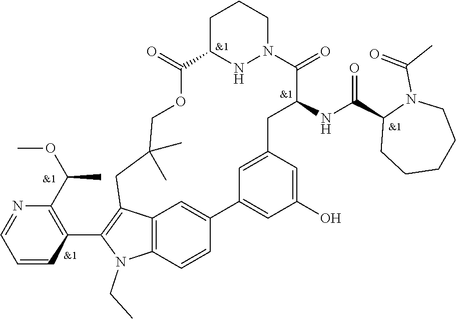 |
| DB3 | 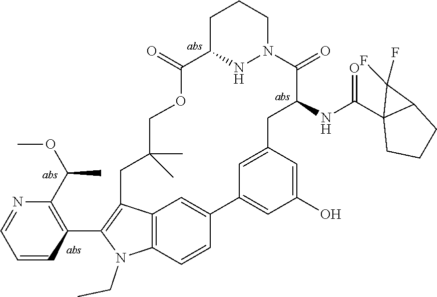 |
| DB4 | 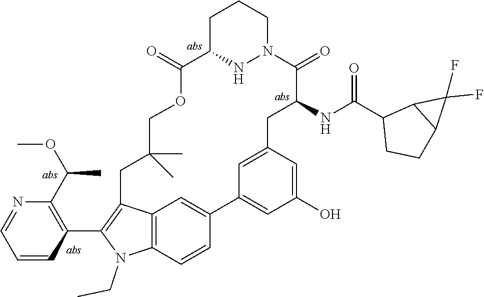 |
| DB5 | 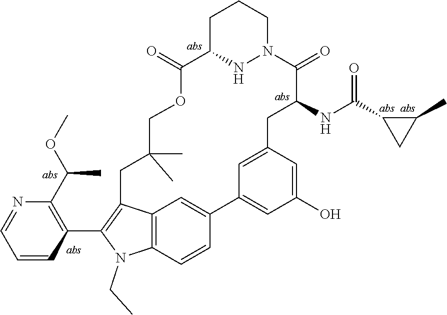 |
| DB6 | 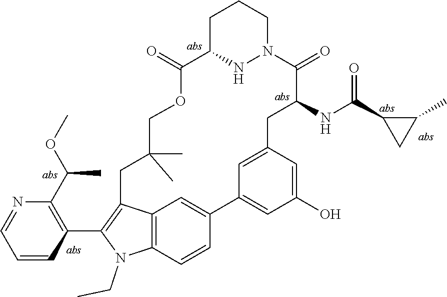 |
| DB7 | 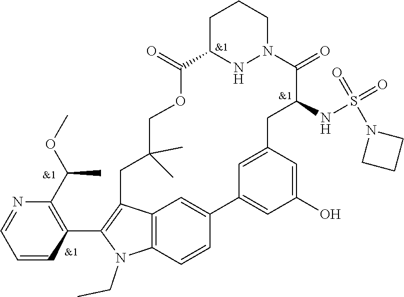 |
| DB8 | 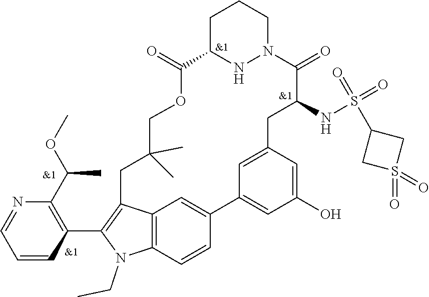 |
| DB9 | 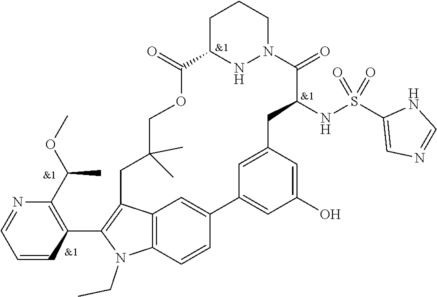 |
| DB10 | 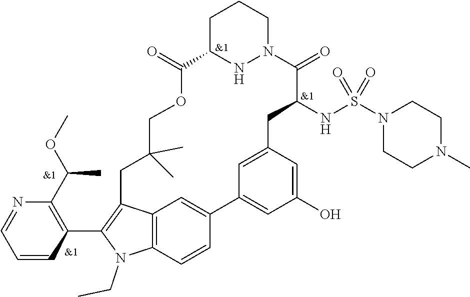 |
| DB11 | 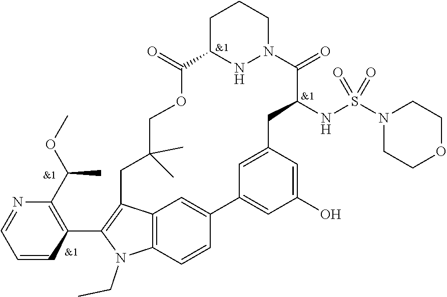 |
| DB12 | 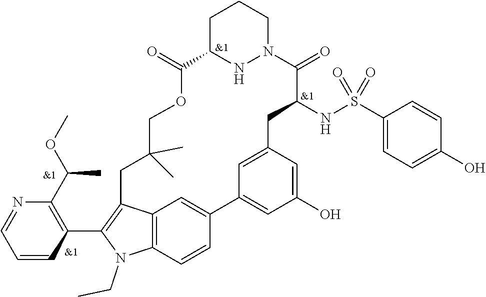 |
| DB13 | 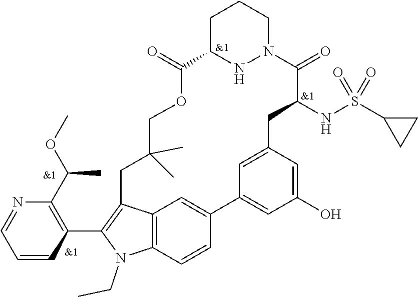 |
| DB14 | 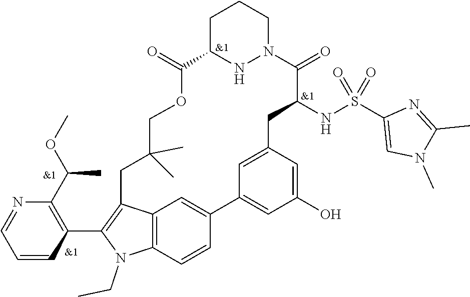 |
| DB15 | 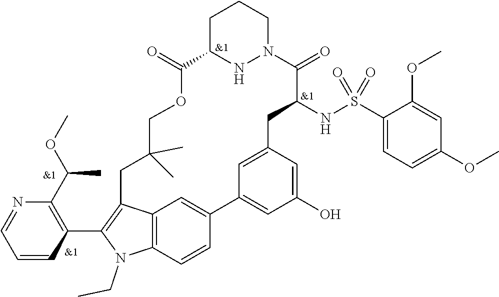 |
| DB16 | 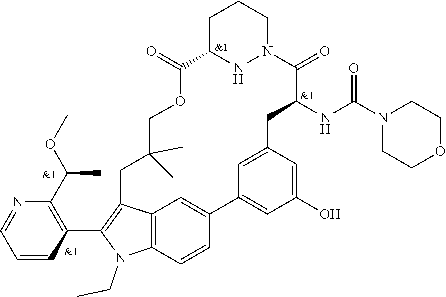 |
| DB17 | 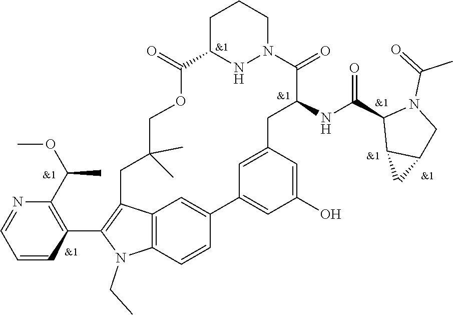 |
| DB18 | 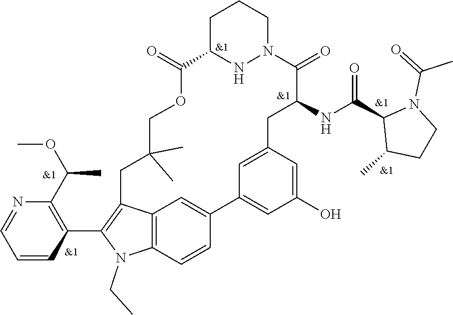 |
| DB19 | 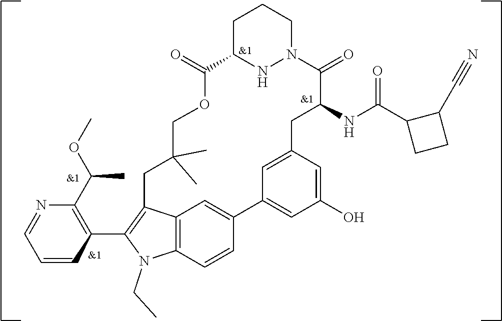 |
| DB20 | 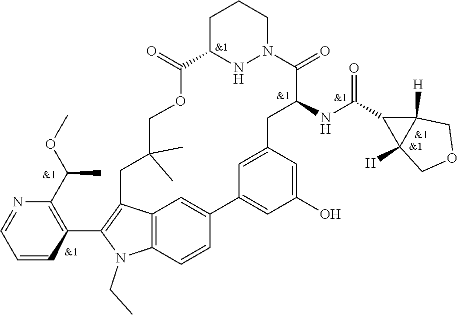 |
| DB21 | 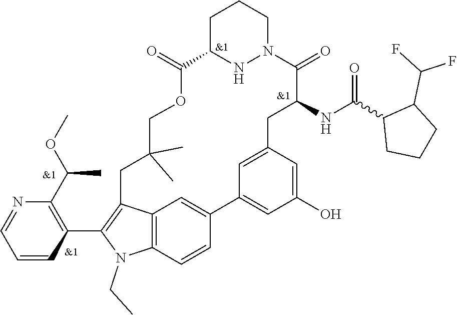 |
| DB22 | 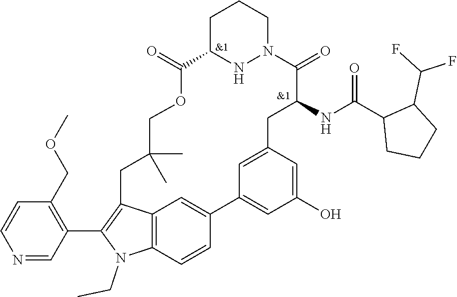 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| DB23 | 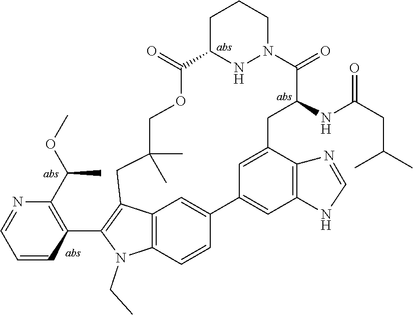 |
| DB24 | 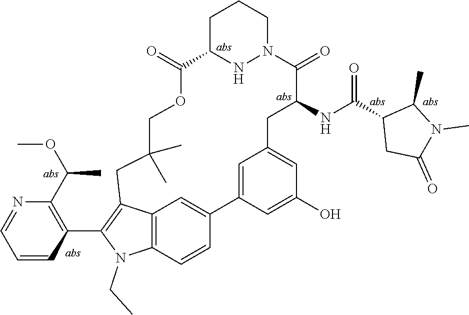 |
| DB25 | 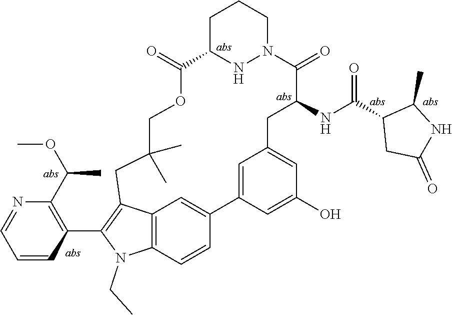 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereo-
chemistry of such diastereomer may not be known.

In some embodiments, a compound of the present invention is a compound selected from Table D3 (e.g., DC1-DC20 or DC1-DC21), or a pharmaceutically acceptable salt or stereoisomer thereof. In some embodiments, a compound of the present invention is a compound selected from Table D3 (e.g., DC1-DC20 or DC1-DC21), or a pharmaceutically acceptable salt or atropisomer thereof.

In some embodiments, a compound of the present invention is not a compound selected from Table D3 (e.g., DC1-DC20 or DC1-DC21). In some embodiments, a compound of the present invention is not a compound selected from Table D3 (e.g., DC1-DC20 or DC1-DC21), or a pharmaceutically acceptable salt or stereoisomer thereof. In some embodiments, a compound of the present invention is not a compound selected from Table D3 (e.g., DC1-DC20 or DC1-DC21), or a pharmaceutically acceptable salt or atropisomer thereof.

TABLE D3
Certain Compounds

| Ex. # | Structure |
|---|---|
| DC1 | 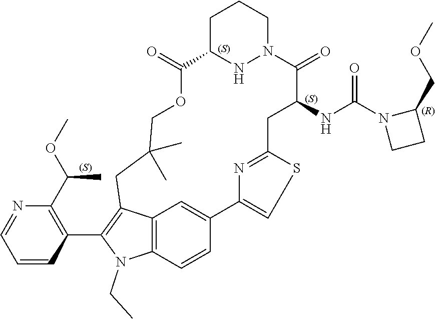 |
| DC2 | 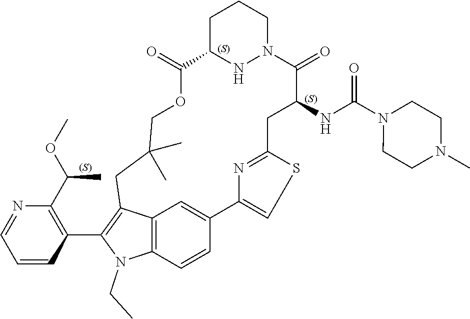 |
| DC3 | 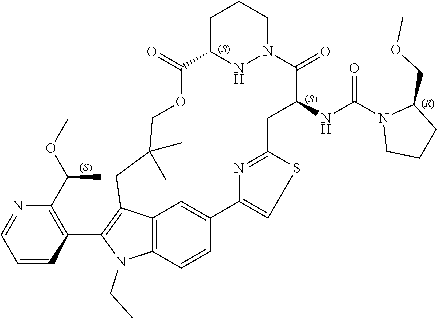 |
| DC4 | 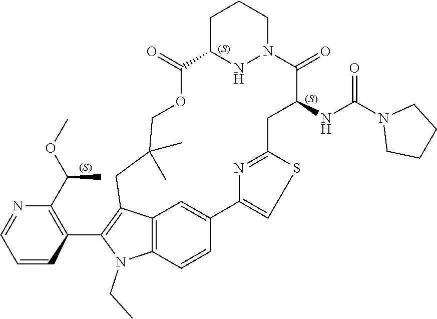 |
| DC5 | 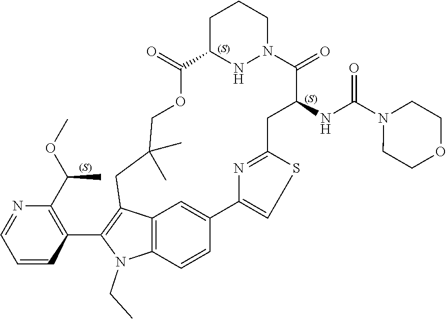 |
| DC6 | 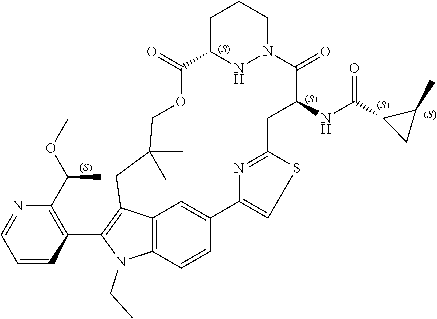 |
| DC7 | 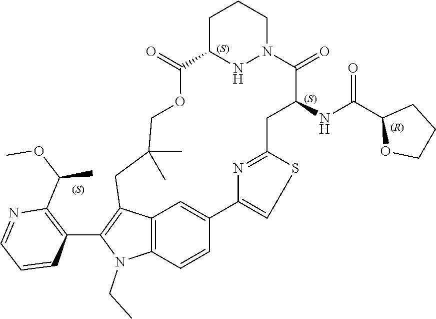 |
| DC8 | 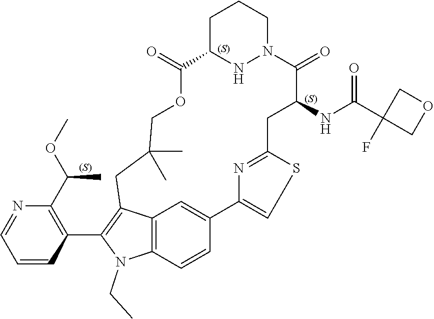 |
| DC9 | 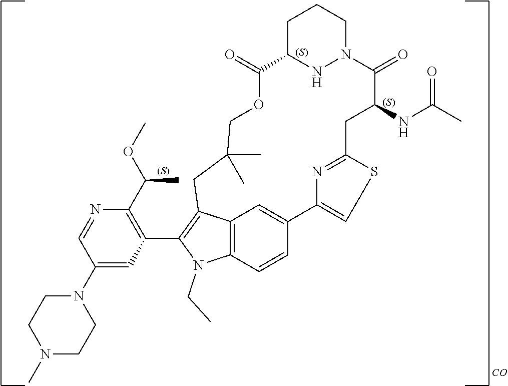 |
| DC10 | 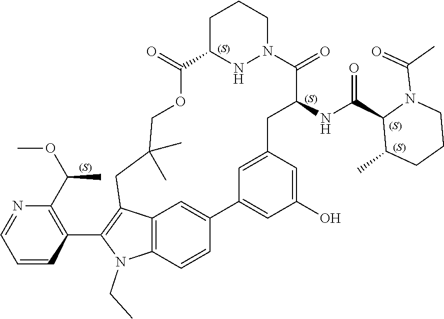 |
| DC11 | 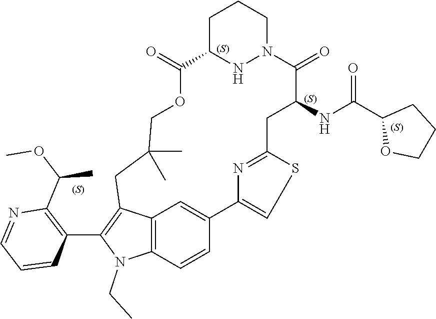 |
| DC12 | 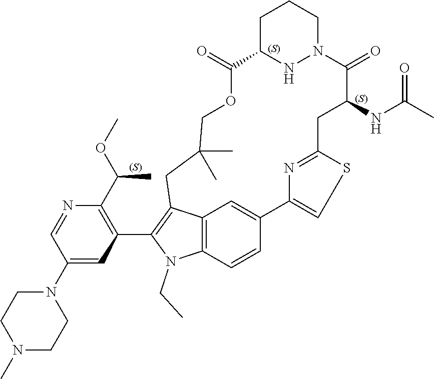 |
| DC13 | 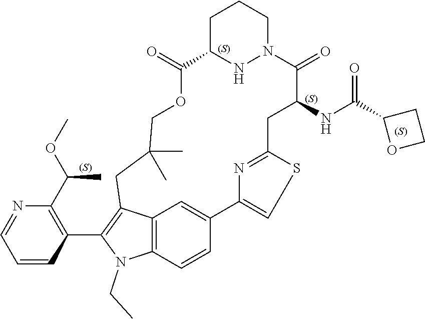 |
| DC14 | 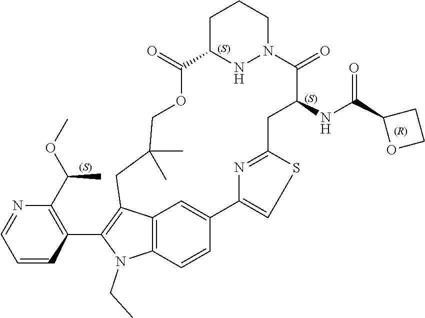 |
| DC15 | 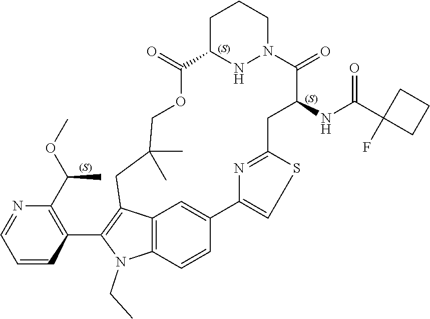 |
| DC16 | 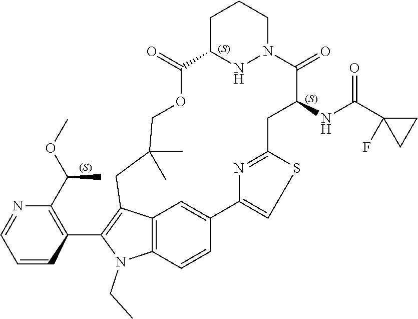 |
| DC17 | 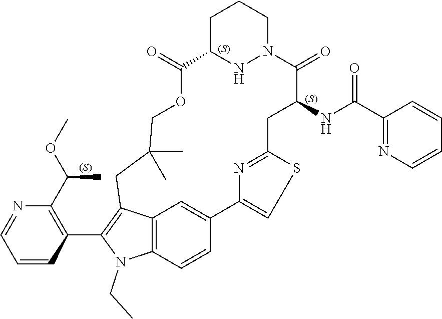 |
| DC18 | 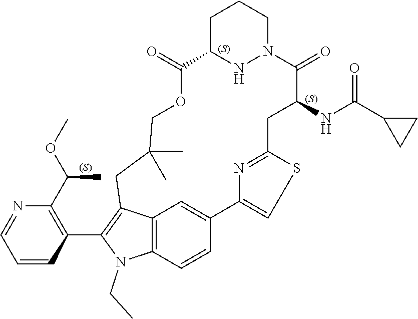 |
| DC19 | 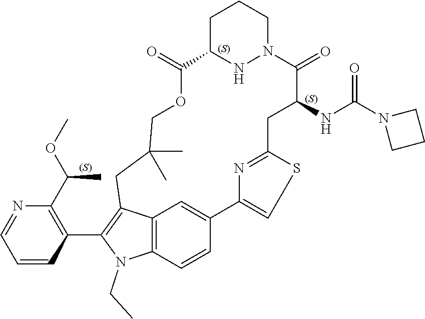 |
| DC20 | 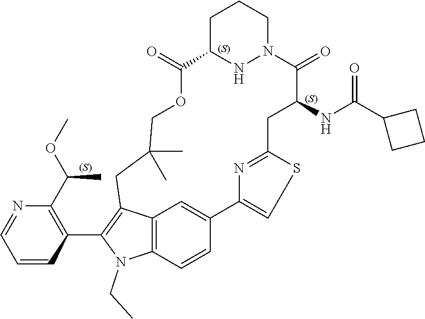 |
| DC21 | 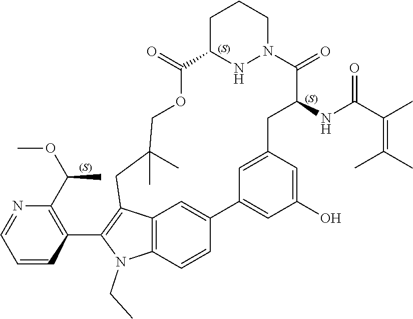 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereo-
chemistry of such diastereomer may not be known.

The compounds described herein in Tables D1a, D1 b, D2, and D3 may be made from commercially available starting materials or synthesized using known organic, inorganic, or enzymatic processes.

The compounds of the present invention in Tables D1a, D1 b, D2, and D3 can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below and in WO 2022/060836.

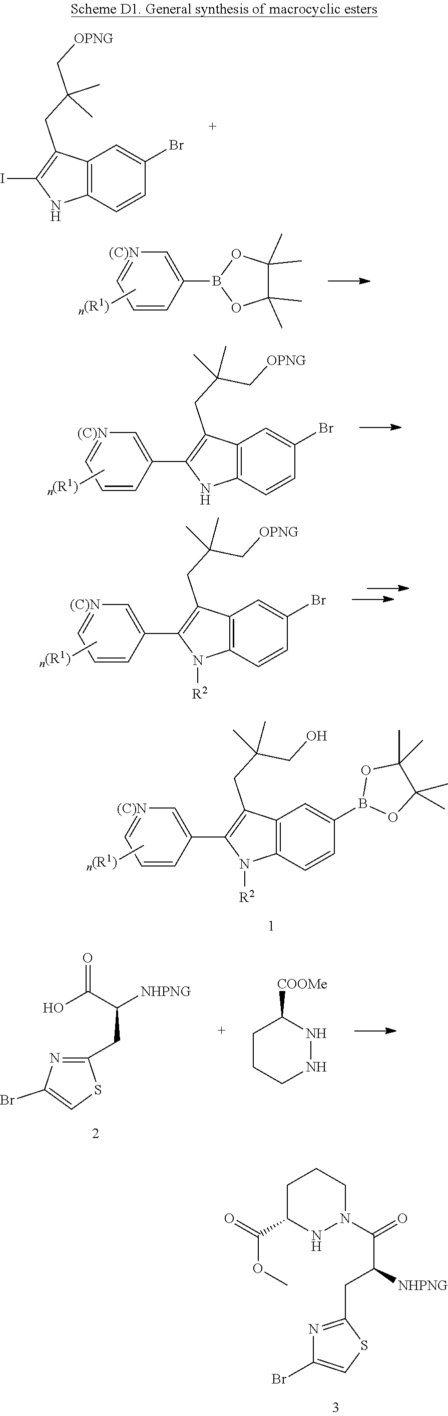

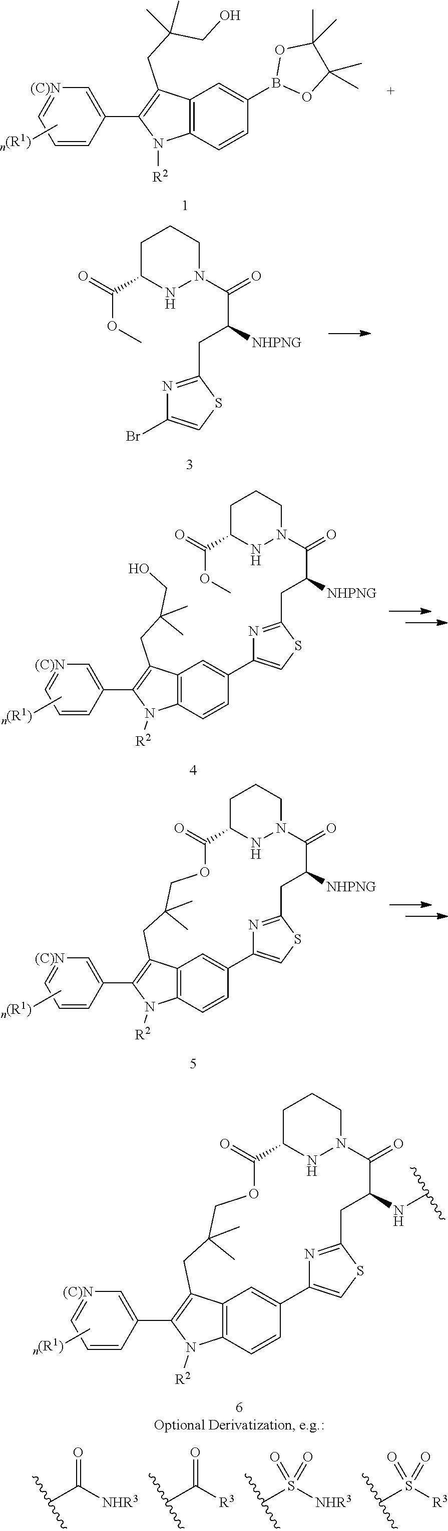

A general synthesis of macrocyclic esters is outlined in Scheme D1. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl-amino-3-(4-bromothiazol-2-yl)propanoyl)hexahydropyridazine-3-carboxylate (3) can be prepared via coupling of (S)-2-amino-3-(4-bromothiazol-2-yl)propanoic acid (2) with methyl (S)-hexahydropyridazine-3-carboxylate.

The final macrocyclic esters can be made by coupling of methyl-amino-3-(4-bromothiazol-2-yl)propanoyl)hexahydropyridazine-3-carboxylate (3) and an appropriately substituted indolyl boronic ester (1) in the presence of Pd catalyst followed by hydrolysis and macrolactonization steps to result in an appropriately protected macrocyclic intermediate (5). Deprotection and coupling with an appropriately substituted carboxylic acid (or other coupling partner) can result in a macrocyclic product. Additional deprotection or functionalization steps could be required to produce a final compound 6.

Further, with respect to Scheme D1, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

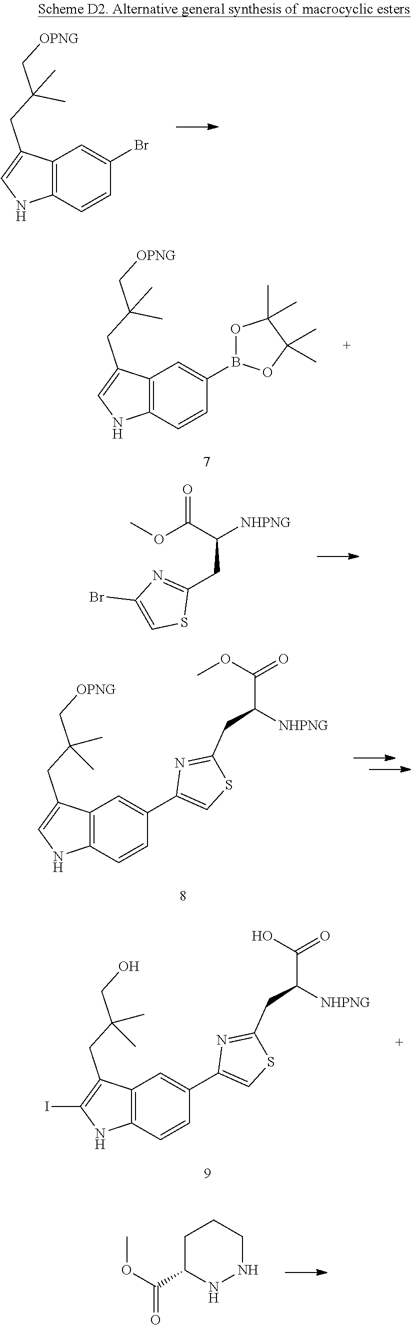

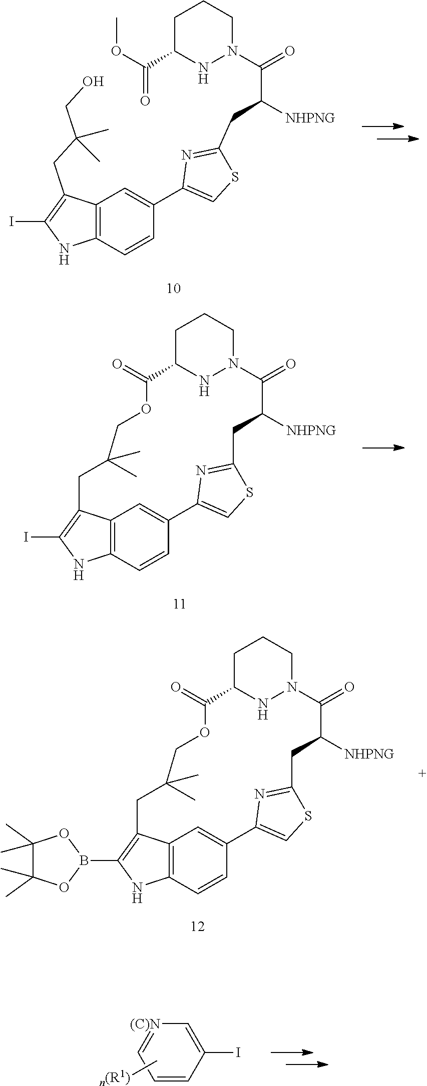

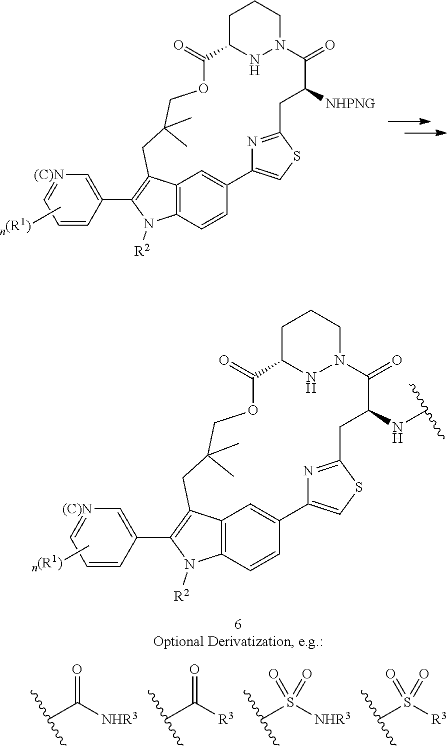

Alternatively, macrocyclic esters can be prepared as described in Scheme D2. An appropriately substituted and protected indolyl boronic ester (7) can be coupled in the presence of Pd catalyst with (S)-2-amino-3-(4-bromothiazol-2-yl)propanoic acid, followed by iodination, deprotection, and ester hydrolysis. Subsequent coupling with methyl (S)-hexahydropyridazine-3-carboxylate, followed by hydrolysis and macrolactonization can result in iodo intermediate (11). Subsequent palladium mediated borylation and coupling in the presence of Pd catalyst with an appropriately substituted iodo aryl or iodo heteroaryl intermediate can yield an appropriately protected macrocyclic intermediate. Alkylation, deprotection and coupling with an appropriately substituted carboxylic acid carboxylic acid (or other coupling partner) results in a macrocyclic product. Additional deprotection or functionalization steps could be required to produce a final compound 6.

Further, with respect to Scheme D2, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

Compounds of Table D1a or Table D1 b herein were prepared using methods disclosed in WO 2022/060836 or were prepared using methods described herein combined with the knowledge of one of skill in the art.

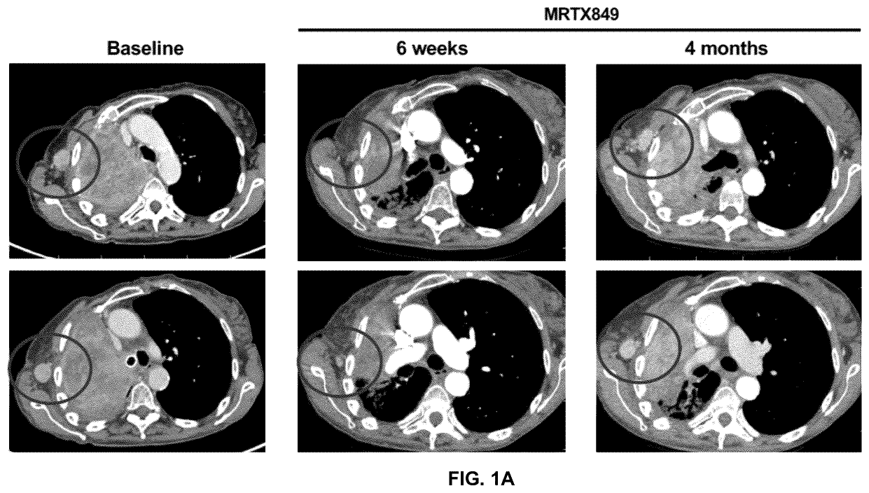

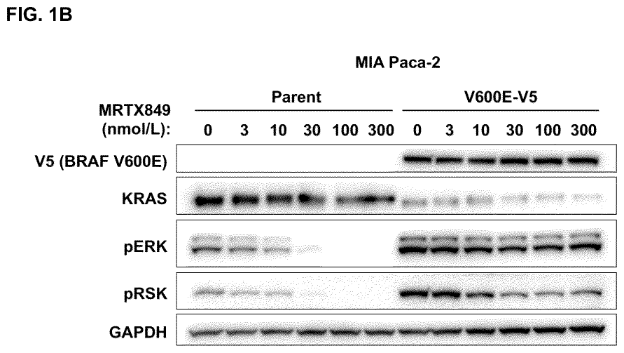

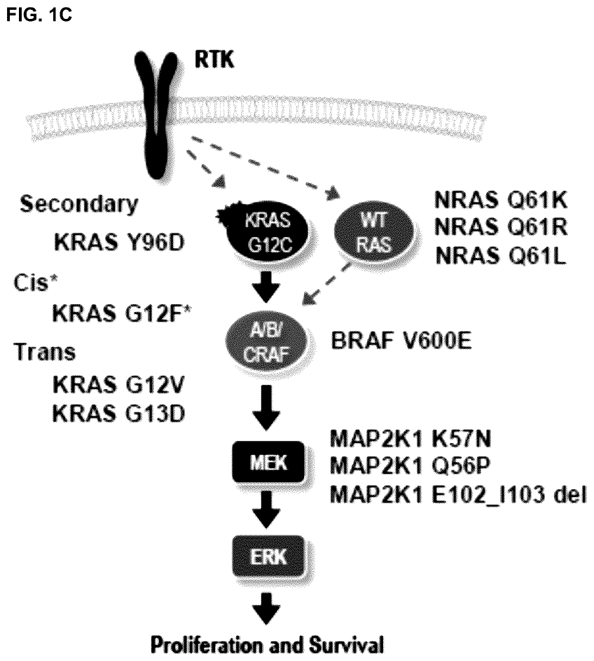

In some embodiments, the RAS(ON) inhibitor is a compound described by a Formula in WO 2020132597, such as a compound of Formula (I) therein, or a pharmaceutically acceptable salt thereof, or FIG. 1 therein, or a pharmaceutically acceptable salt thereof.

In some embodiments, the RAS(ON) inhibitor is RM-018, which is a RAS(ON)G12C inhibitor compound of Formula BI herein, and also a compound of Table B1 herein, and is also found in WO 2021/091982. “RM-018,” as referred to herein, means the following compound:

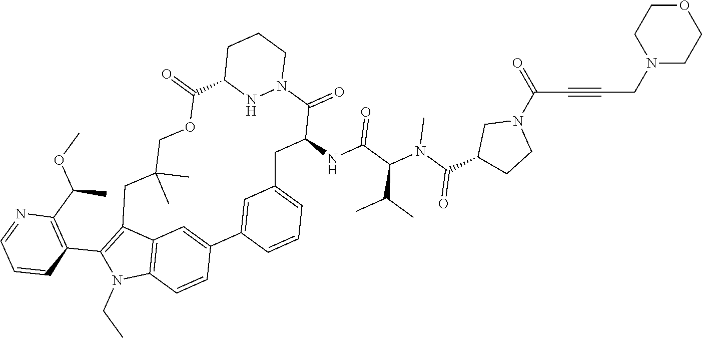

In some embodiments, a RAS(ON) inhibitor described herein entails formation of a high affinity three-component complex between a synthetic ligand and two intracellular proteins which do not interact under normal physiological conditions: the target protein of interest (e.g., RAS), and a widely expressed cytosolic chaperone (presenter protein) in the cell (e.g., cyclophilin A). More specifically, in some embodiments, the RAS(ON) inhibitors described herein induce a new binding pocket in RAS by driving formation of a high affinity tri-complex between the RAS protein and the widely expressed cytosolic chaperone, cyclophilin A (CYPA). Without being bound by theory, one way the inhibitory effect on Ras is affected by compounds of the invention and the complexes they form is by steric occlusion of the interaction site between Ras and downstream effector molecules, such as RAF and PI3K, which are required for propagating the oncogenic signal.

Without being bound by theory, both covalent and non-covalent interactions of a RAS(ON) inhibitor described herein with Ras and the chaperone protein (e.g., cyclophilin A) may contribute to the inhibition of Ras activity. In some embodiments, a RAS(ON) inhibitor described herein forms a covalent adduct with a side chain of a Ras protein (e.g., a sulfhydryl side chain of the cysteine at position 12 or of a mutant Ras protein). Covalent adducts may also be formed with other side chains of Ras. In addition, or alternatively, non-covalent interactions may be at play: for example, van der Waals, hydrophobic, hydrophilic and hydrogen bond interactions, and combinations thereof, may contribute to the ability of the compounds of the present invention to form complexes and act as Ras inhibitors. Accordingly, a variety of Ras proteins may be inhibited by RAS(ON) inhibitors described herein (e.g., K-Ras, N-Ras, H-Ras, and mutants thereof at positions 12, 13 and 61, such as G12C, G12D, G12V, G12S, G13C, G13D, and Q61L, and others described herein).

Methods of determining covalent adduct formation are known in the art and are described in, for example, WO 2021/091982 and WO 2021/091967.

### RAS(OFF) Inhibitors

RAS(OFF) inhibitors are provided herein and are known to those of skill in the art. A RAS(OFF) inhibitor refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS).

In some embodiments, the RAS(OFF) inhibitor is selective for RAS that includes an amino acid substitution at G12, G13, Q61, or a combination thereof. In some embodiments, the RAS(OFF) inhibitor is selective for RAS that includes an amino acid substitution selected from G12C, G12D, G12V, G13C, G13D, Q61 L, or a combination thereof. In some embodiments, the RAS(OFF) inhibitor is selective for RAS that includes a G12C or G12D amino acid substitution.

In some embodiments, the RAS(OFF) inhibitor is a KRAS(OFF) inhibitor, where a KRAS(OFF) inhibitor refers to an inhibitor that targets, that is, selectively binds to or inhibits the GDP-bound, inactive state of KRAS (e.g., selective over the GTP-bound, active state of KRAS). In some embodiments, the KRAS(OFF) inhibitor is selective for KRAS that includes an amino acid substitution at G12, G13, Q61, A146, K117, L19, Q22, V14, A59, or a combination thereof. In some embodiments, the KRAS(OFF) inhibitor is selective for KRAS that includes an amino acid substitution selected from G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, G13V, or a combination thereof.

In some embodiments, the RAS(OFF) inhibitor is an NRAS(OFF) inhibitor, where an NRAS(OFF) inhibitor refers to an inhibitor that targets, that is, selectively binds to or inhibits the GDP-bound, inactive state of NRAS (e.g., selective over the GTP-bound, active state of NRAS). In some embodiments, the NRAS(OFF) inhibitor is selective for NRAS that includes an amino acid substitution at G12, G13, Q61, P185, A146, G60, A59, E132, E49, T50, or a combination thereof. In some embodiments, the NRAS(OFF) inhibitor is selective for NRAS that includes an amino acid substitution selected from Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, A59T, or a combination thereof.

In some embodiments, the RAS(OFF) inhibitor is an HRAS(OFF) inhibitor, where an HRAS(OFF) inhibitor refers to an inhibitor that targets, that is, selectively binds to or inhibits the GDP-bound, inactive state of HRAS (e.g., selective over the GTP-bound, active state of HRAS). In some embodiments, the HRAS(OFF) inhibitor is selective for HRAS that includes an amino acid substitution at G12, G13, Q61, K117, A59, A18, D119, A66, A146, or a combination thereof. In some embodiments, the HRAS(OFF) inhibitor is selective for NRAS that includes an amino acid substitution selected from Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, G12R, or a combination thereof.

In some embodiments, the RAS(OFF) inhibitor is a compound disclosed in any one of the following patent publications: WO 2022052895, WO 2022048545, WO 2022047093, WO 2022042630, WO 2022040469, WO 2022037631, WO 2022037560, WO 2022031678, WO 2022028492, WO 2022028346, WO 2022026726, WO 2022026723, WO 2022015375, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, CN 114195788, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113248521, CN 113087700, CN 113024544, CN 113004269, CN 112920183, CN 112390818, CN 112390788, CN 112300194, CN 112300173, CN 112225734, CN 112142735, CN 112110918, CN 112094269, CN 112047937, and CN 109574871, each of which is incorporated herein by reference in its entirety.

In some embodiments, the RAS(OFF) inhibitor is selected from AMG 510 (sotorasib), MRTX849 (adagrasib), MRTX1257, JNJ-74699157 (ARS-3248), LY3537982, LY3499446, ARS-853, ARS-1620, GDC-6036, JDQ443, BPI-421286, and JAB-21000. In some embodiments, the RAS(OFF) inhibitor is an inhibitor of K-Ras G12D, such as MRTX1133 or JAB-22000. In some embodiments, the RAS(OFF) inhibitor is a K-Ras G12V inhibitor, such as JAB-23000.

Reference to “AMG 510,” “MRTX849,” “MRTX1257,” “ARS-853”, “ARS-1620”, and “MRTX1133” means the following compounds:

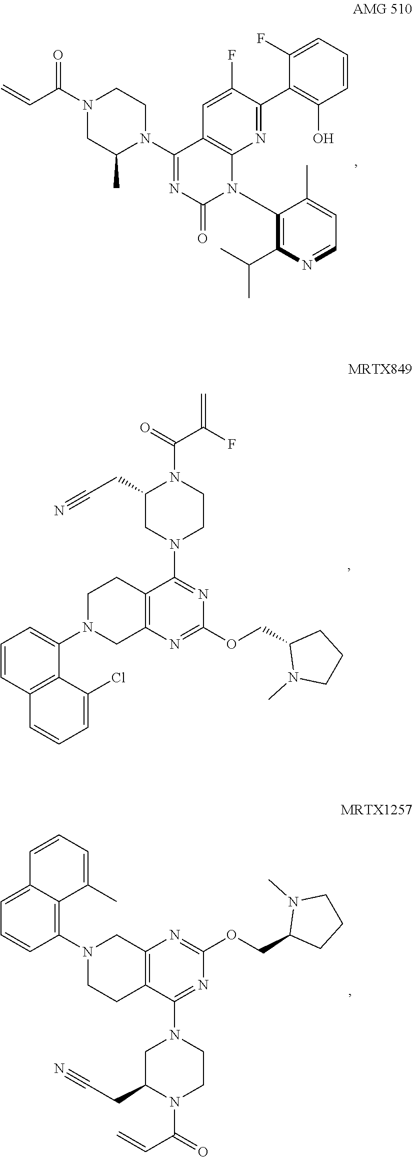

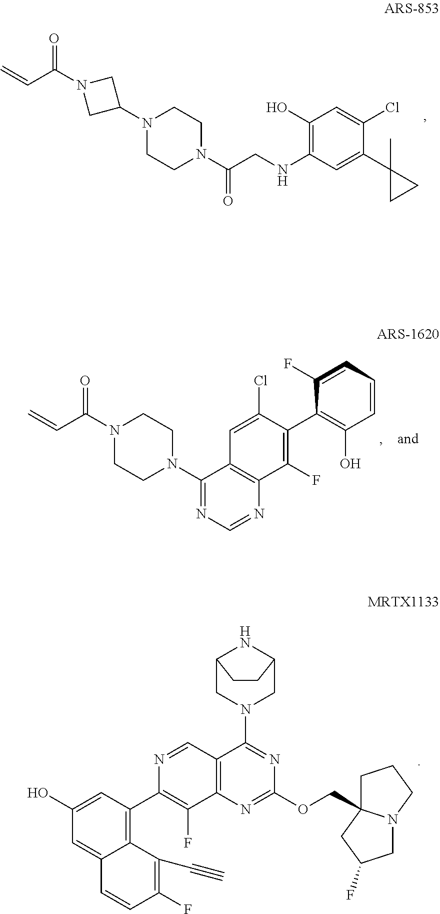

In any embodiment herein regarding a RAS(OFF) inhibitor, the RAS(OFF) inhibitor may be substituted by a RAS inhibitor disclosed in the following patent publication: WO 2021/041671, which is incorporated herein by reference in its entirety. In some embodiments, such a substituted RAS inhibitor is MRTX1133.

### Pharmaceutical Compositions

The disclosure provides pharmaceutical compositions including one or more RAS inhibitor compounds, or a pharmaceutically acceptable salt thereof, and a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Compounds described herein, whether expressly stated or not, may be provided or utilized in salt form, e.g., a pharmaceutically acceptable salt form, unless expressly stated to the contrary.

The compounds of the disclosure may have ionizable groups so as to be capable of preparation as pharmaceutically acceptable salts. These salts may be acid addition salts involving inorganic or organic acids or the salts may, in the case of acidic forms of the compounds of the disclosure, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

For use as treatment of subjects, the compounds of the disclosure, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present disclosure, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the disclosure, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present disclosure can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the disclosure, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further include an additional compound having antiproliferative (e.g., anti-cancer) activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present disclosure can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Methods of Use

In some embodiments, the disclosure provides a method of treating a disease or disorder that is characterized by aberrant RAS activity due to one or more RAS mutations. In some embodiments, the disease or disorder is a cancer (e.g., a cancer having one or more RAS mutations that cause aberrant RAS activity).

As described herein, cancer cells treated with a RAS(OFF) inhibitor may develop resistance, e.g., through the acquisition of one or more mutations that render the RAS(OFF) inhibitor less effective or ineffective. The present disclosure is based, at least in part, on the observation that some cancers resistant to treatment with a RAS(OFF) inhibitor remain responsive to treatment with a RAS(ON) inhibitor. Thus, administering a RAS(ON) inhibitor to a subject having cancer can slow or halt oncogenic signaling or disease progression where the cancer is resistant to treatment with a RAS(OFF) inhibitor. Additionally, administration of a RAS(ON) inhibitor, e.g., administered in combination with a RAS(OFF) inhibitor, may prevent the acquisition of one or more mutations in RAS that confer resistance to the RAS(OFF) inhibitor.

Accordingly, the disclosure provides a method of treating cancer in a subject in need thereof, the method including administering to the subject a therapeutically effective amount of one or more compounds described here, or a pharmaceutically acceptable salt thereof, or a pharmaceutical composition including one or more compounds described herein or salts thereof.

The disclosure also provides a method of treating cancer in a subject in need thereof, wherein the cancer includes a mutation in RAS and the cancer is resistant to treatment with a RAS(OFF) inhibitor, the method including administering to the subject a RAS(ON) inhibitor. In some embodiments, the RAS mutation is an amino acid substitution at Y96. In some embodiments, the amino acid substitution is Y96D.

The disclosure also provides a method of treating cancer in a subject in need thereof, wherein the cancer includes an amino acid substitution at RAS Y96, the method including administering to the subject a RAS(ON) inhibitor. In some embodiments, the amino acid substitution is Y96D.

In some embodiments, the method further includes administering to the subject a RAS(OFF) inhibitor (e.g., a RAS(OFF) inhibitor is administered to the subject in combination with the RAS(ON) inhibitor). The RAS(ON) inhibitor and the RAS(OFF) inhibitor may be administered simultaneously or sequentially. The RAS(ON) inhibitor and the RAS(OFF) inhibitor may be administered as a single formulation or in separate formulations. In some embodiments, the RAS(OFF) inhibitor is administered for a first period of time; and the RAS(ON) inhibitor is administered for a second period of time, wherein the first period of time and the second period of time do not overlap and the first period of time precedes the second period of time. In some embodiments, the RAS(OFF) inhibitor is administered for a first period of time; and the RAS(OFF) inhibitor and RAS(ON) inhibitor are administered for a second period of time, wherein the first period of time and the second period of time do not overlap and the first period of time precedes the second period of time. In some embodiments, the first period of time is a period of time sufficient to acquire a mutation (e.g., a RAS mutation) that confers resistance to treatment with the RAS(OFF) inhibitor. In some embodiments, the first period of time is between one week and one month, between one week and six months, between one week and one year, between one month and six months, between one month and one year, between one month and two years, between one month and five years, at least one week, at least one month, at least six months, or at least one year. In some embodiments, the second period of time is between one week and one month, between one week and six months, between one week and one year, between one month and six months, between one month and one year, between one month and two years, between one month and five years, at least one week, at least one month, at least six months, or at least one year.

In some embodiments, the subject's cancer progresses on the RAS(OFF) inhibitor (e.g., when the subject is administered the RAS(OFF) inhibitor in the absence of a RAS(ON) inhibitor). Disease progression of a cancer (e.g., a cancer described herein) can be evaluated by any one or more of several established methods. A person of skill in the art can monitor a subject by direct observation in order to evaluate how the symptoms exhibited by the subject have changed (e.g., a decrease or absence of symptoms) in response to a treatment (e.g., a method of treatment disclosed herein). A subject may also be examined by MRI, CT scan, or PET analysis in order to determine if a tumor has metastasized or if the size of a tumor has changed (e.g., decreased in response to a treatment (e.g., a method of treatment described herein)). Optionally, cells can be extracted from the subject through a biopsy or procedure or tumor DNA can be isolated from the blood of a subject, and a quantitative biochemical analysis can be conducted in order to assess the relative cancer burden and determine the presence or emergence of specific mutations possibly involved in resistance. Based on the results of these analyses, a person of skill in the art may prescribe higher/lower dosages or more/less frequent dosing of a treatment in subsequent rounds of treatment.

In some embodiments, the subject has been treated with a RAS(OFF) inhibitor (e.g., the subject has been previously treated with a RAS(OFF) inhibitor, e.g., prior to administration of the RAS(ON) inhibitor). In some embodiments, the subject has acquired resistance to a RAS(OFF) inhibitor (e.g., has acquired a mutation that confers resistance to a RAS(OFF) inhibitor, e.g., prior to administration of the RAS(ON) inhibitor).

In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma.

In some embodiments, the compounds of the present disclosure or pharmaceutically acceptable salts thereof, pharmaceutical compositions including such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions including such compounds or salts, and methods of the disclosure include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the cancer includes a RAS mutation, such as a RAS mutation described herein. In some embodiments, a mutation is selected from:
(a) the following KRAS mutants: G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
(b) the following HRAS mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following NRAS mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof;
   or a combination of any of the foregoing. In some embodiments, the cancer includes a KRAS mutation selected from the group consisting of G12C, G12D, G13C, G12V, G13D, G12R, G12S, Q61H, Q61K and Q61 L. In some embodiments, the cancer includes an NRAS mutation selected from the group consisting of G12C, Q61H, Q61K, Q61 L, Q61P and Q61R. In some embodiments, the cancer includes an HRAS mutation selected from the group consisting of Q61H and Q61 L. In some embodiments, the cancer includes a RAS mutation selected from the group consisting of G12C, G13C, G12A, G12D, G13D, G12S, G13S, G12V and G13V. In some embodiments, the cancer includes at least two RAS mutations selected from the group consisting of G12C, G13C, G12A, G12D, G13D, G12S, G13S, G12V and G13V. In some embodiments, a compound of the present disclosure inhibits more than one RAS mutant. For example, a compound may inhibit both KRAS G12C and KRAS G13C. A compound may inhibit both NRAS G12C and KRAS G12C. In some embodiments, a compound may inhibit both KRAS G12C and KRAS G12D. In some embodiments, a compound may inhibit both KRAS G12V and KRAS G12C. In some embodiments, a compound may inhibit both KRAS G12V and KRAS G12S. In some embodiments, a compound of the present disclosure inhibits RAS^{amp} in addition to one or more additional RAS mutations (e.g., K-, H- or NRAS^{amp} and KRAS G12D, G12V, G12C, G13D, G12R, G12A, Q61H, G12S, A146T, G13C, Q61 L, Q61R, K117N, A146V, G12F, Q61K, L19F, Q22K, V141, A59T, A146P, G13R, G12L, or G13V; K-, H- or NRAS^{amp} and HRAS Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K-, H- or NRAS^{amp} and NRAS Q61R, Q61K, G12D, Q61 L, Q61H, G13R, G13D, G12S, G12C, G12V, G12A, G13V, G12R, P185S, G13C, A146T, G60E, Q61P, A59D, E132K, E49K, T501, A146V, or A59T).

Methods of detecting RAS mutations are known in the art. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

In some embodiments, the cancer is non-small cell lung cancer and the RAS mutation includes a KRAS mutation, such as KRAS G12C, KRAS G12V or KRAS G12D. In some embodiments, the cancer is colorectal cancer and the RAS mutation includes a KRAS mutation, such as KRAS G12C, KRAS G12V or KRAS G12D. In some embodiments, the cancer is pancreatic cancer and the RAS mutation includes an KRAS mutation, such as KRAS G12D or KRAS G12V. In some embodiments, the cancer is pancreatic cancer and the RAS mutation includes an NRAS mutation, such as NRAS G12D. In some embodiments, the cancer is melanoma and the RAS mutation includes an NRAS mutation, such as NRAS Q61R or NRAS Q61K. In some embodiments, the cancer is non-small cell lung cancer and the Ras protein is K-Ras^{amp}. In any of the foregoing if not already specified, a compound may inhibit Ras^{WT}(e.g., K-, H- or N-Ras^{WT}) or Ras^{amp} (e.g., K-, H- or N-Ras^{amp}) as well.

In some embodiments, a cancer includes a RAS mutation and an STK11^{LOF}, a KEAP1, an EPHA5 or an NF1 mutation. In some embodiments, the cancer is non-small cell lung cancer and includes a KRAS G12C mutation. In some embodiments, the cancer is non-small cell lung cancer and includes a KRAS G12C mutation and an STK11^{LOF} mutation. In some embodiments, the cancer is non-small cell lung cancer and includes a KRAS G12C mutation and an STK11^{LOF} mutation. In some embodiments, a cancer includes a KRAS G13C RAS mutation and an STK11^{LOF} a KEAP1, an EPHA5 or an NF1 mutation. In some embodiments, the cancer is non-small cell lung cancer and includes a KRAS G12D mutation. In some embodiments, the cancer is non-small cell lung cancer and includes a KRAS G12V mutation. In some embodiments, the cancer is colorectal cancer and includes a KRAS G12C mutation. In some embodiments, the cancer is pancreatic cancer and includes a K-Ras G12C or KRAS G12D mutation. In some embodiments, the cancer is pancreatic cancer and includes a KRAS G12V mutation. In some embodiments, the cancer is endometrial cancer and includes a KRAS G12C mutation. In some embodiments, the cancer is gastric cancer and includes a KRAS G12C mutation. In any of the foregoing, a compound may inhibit Ras^{WT} (e.g., K-, H- or N-Ras^{WT}) or Ras^{amp} (e.g., K-, H- or N-Ras^{amp}) as well.

Methods for detecting a mutation in a KRAS, HRAS or NRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12C KRAS, HRAS or NRAS mutations by real-time PCR. In real-time PCR, fluorescent probes specific for the KRAS, HRAS or NRAS G12C mutation are used. When a mutation is present, the probe binds and fluorescence is detected. In some embodiments, the KRAS, HRAS or NRAS G12C mutation is identified using a direct sequencing method of specific regions (e.g., exon 2 or exon 3) in the KRAS, HRAS or NRAS gene. This technique will identify all possible mutations in the region sequenced.

Methods for detecting a mutation in a KRAS, HRAS or NRAS protein are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing.

Methods for determining whether a tumor or cancer includes a G12C or other KRAS, HRAS or NRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

Also provided is a method of inhibiting a RAS protein in a cell, the method including contacting the cell with an effective amount of a compound of the present disclosure, or a pharmaceutically acceptable salt thereof. The disclosure also provides a method of inhibiting RAS in a cell, wherein the RAS includes an amino acid substitution at Y96, the method including contacting the cell with a RAS(ON) inhibitor. In some embodiments, the amino acid substitution is Y96D. The cell may be a cancer cell. The cancer cell may be of any type of cancer described herein. The cell may be in vivo or in vitro.

### Combination Therapies

The methods of the disclosure may include a compound of the disclosure used alone or in combination with one or more additional therapies (e.g., non-drug treatments or therapeutic agents).

In particular, the disclosure provides methods of treatment that include administering (e.g., to a subject or a cell) a RAS(ON) inhibitor with one or more additional therapies (e.g., one or more additional cancer therapies described herein). In some embodiments, a RAS(ON) inhibitor is administered in combination with a RAS(OFF) inhibitor. In some embodiments, a RAS(ON) inhibitor is administered in combination with a RAS(OFF) inhibitor and one or more additional therapies (e.g., one or more additional cancer therapies described herein).

As described herein, “in combination,” includes administration of two or more therapies as part of a therapeutic regimen. The therapies may be administered simultaneously or sequentially. Such sequential administration may be close or remote in time. Where the therapies are therapeutic agents, the therapeutic agents may be formulated together as a single dosage form or formulated as separate dosage forms. The therapeutic agents may be administered by the same route of administration or by different routes of administration.

When a RAS(ON) inhibitor is administered in combination with one or more additional therapies, the RAS(ON) inhibitor may be administered before, after, or concurrently with one or more of such additional therapies.

The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the compounds of the present disclosure can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

In this combination therapy section, all references are incorporated by reference for the agents described, or a pharmaceutically acceptable salt, solvate, isomer (e.g., stereoisomer), prodrug, or tautomer thereof, whether explicitly stated as such or not.

Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the disclosure may be used as an adjuvant therapy after surgery. In some embodiments, the compounds of the disclosure may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamm a radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present disclosure can render abnormal cells more sensitive to treatment with radiation for purposes of killing or inhibiting the growth of such cells. Accordingly, this disclosure further relates to a method for sensitizing abnormal cells in a mammal to treatment with radiation which includes administering to the mammal an amount of a compound of the present disclosure, which amount is effective to sensitize abnormal cells to treatment with radiation. The amount of the compound in this method can be determined according to the means for ascertaining effective amounts of such compounds described herein. In some embodiments, the compounds of the present disclosure may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

Therapeutic Agents

A therapeutic agent may be a compound used in the treatment of cancer or symptoms associated therewith.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present disclosure include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PDL-1. In some embodiments, the checkpoint inhibitor is an inhibitor or antagonist (e.g., an inhibitory antibody or Fc fusion or small molecule inhibitor) of PDL-2 (e.g., a PDL-2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. Nat. Rev. Neurol. 11(9):504-514 (2015), including, without limitation, ipilimumab, tremelimumab, nivolumab, pembrolizumab, AMP224, AMP514/MED10680, BMS936559, MED14736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab).

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-(1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Noivadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin@), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK4/6 inhibitor such as abemaciclib, ribociclib, palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, ridaforolimus, and sirolimus), KSP(Eg5) inhibitors (e.g., Array 520), DNA binding agents (e.g., Zalypsis@), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, the anti-cancer agent is a HER2 inhibitor. Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, RLY-1971, BBP-398; see also Wu et al., Curr Med Chem (2020) 27:1; world wide web at doi.org/10.2174/1568011817666200928114851), a SOS1 inhibitor (e.g., BI-1701963, BI-3406), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312.

In some embodiments, an anti-cancer agent is a SOS1 inhibitor. In some embodiments, the SOS1 inhibitor is selected from those disclosed in WO 2022028506, WO 2022026465, WO 2022017339, WO 2022017519, WO 2021249519, WO 2021249575, WO 2021228028, WO 2021225982, WO 2021203768, WO 2021173524, WO 2021130731, WO 2021127429, WO 2021092115, WO 2021105960, WO 2021074227, WO 2020180768, WO 2020180770, WO 2020173935, WO 2020146470, WO 2019201848, WO 2019122129, WO 2018172250, WO 2018115380, CN 113912608, CN 1138010114, CN 113200981, and US 20210338694, or a pharmaceutically acceptable salt, solvate, isomer (e.g., stereoisomer), prodrug, or tautomer thereof.

In some embodiments, an anti-cancer agent is an additional Ras inhibitor. In some embodiments, the Ras inhibitor targets Ras in its active, or GTP-bound state. In some embodiments, the Ras inhibitor targets Ras in its inactive, or GDP-bound state. In some embodiments, the Ras inhibitor is, such as an inhibitor of K-Ras G12C, such as AMG 510 (sotorasib), MRTX1257, MRTX849 (adagrasib), JNJ-74699157, LY3499446, ARS-1620, ARS-853, BPI-421286, LY3537982, JDQ443, JAB-21000, RMC-6291 or GDC-6036, or a pharmaceutically acceptable salt, solvate, isomer (e.g., stereoisomer), prodrug, or tautomer thereof. In some embodiments, the Ras inhibitor is an inhibitor of K-Ras G12D, such as MRTX1133 or JAB-22000, or a pharmaceutically acceptable salt, solvate, isomer (e.g., stereoisomer), prodrug, or tautomer thereof. In some embodiments, the Ras inhibitor is a K-Ras G12V inhibitor, such as JAB-23000, or a pharmaceutically acceptable salt, solvate, isomer (e.g., stereoisomer), prodrug, or tautomer thereof. In some embodiments, the Ras inhibitor is RMC-6236, or a pharmaceutically acceptable salt, solvate, isomer (e.g., stereoisomer), prodrug, or tautomer thereof. In some embodiments, the Ras inhibitor is selected from a Ras(ON) inhibitor disclosed in the following, incorporated herein by reference in their entireties, or a pharmaceutically acceptable salt, solvate, isomer (e.g., stereoisomer), prodrug, or tautomer thereof: WO 2022/060836, WO 2021091982, WO 2021091967, WO 2021091956 and WO 2020132597. Other examples of Ras inhibitors that may be combined with a Ras inhibitor of the present invention are provided in the following, incorporated herein by reference in their entireties: WO 2022026723, WO 2022015375, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, WO 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, CN 114195788, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113248521, CN 113087700, CN 113024544, CN 113004269, CN 112920183, CN 112390818, CN 112390788, CN 112300194, CN 112300173, CN 112225734, CN 112142735, CN 112110918, CN 112094269, CN 112047937, and CN 109574871, or a pharmaceutically acceptable salt, solvate, isomer (e.g., stereoisomer), prodrug, or tautomer thereof.

In some embodiments, a therapeutic agent that may be combined with a compound of the present disclosure is an inhibitor of the MAP kinase (MAPK) pathway (or “MAPK inhibitor”). MAPK inhibitors include, but are not limited to, one or more MAPK inhibitor described in Cancers (Basel) 2015 Sep.; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 Sep.; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies. In some embodiments, a therapeutic agent may be a pan-RTK inhibitor, such as afatinib.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. In some embodiments, the EGFR inhibitor is osimertinib (Tagrisso®). Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4).

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl] phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[l-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl): 3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552, having the structure

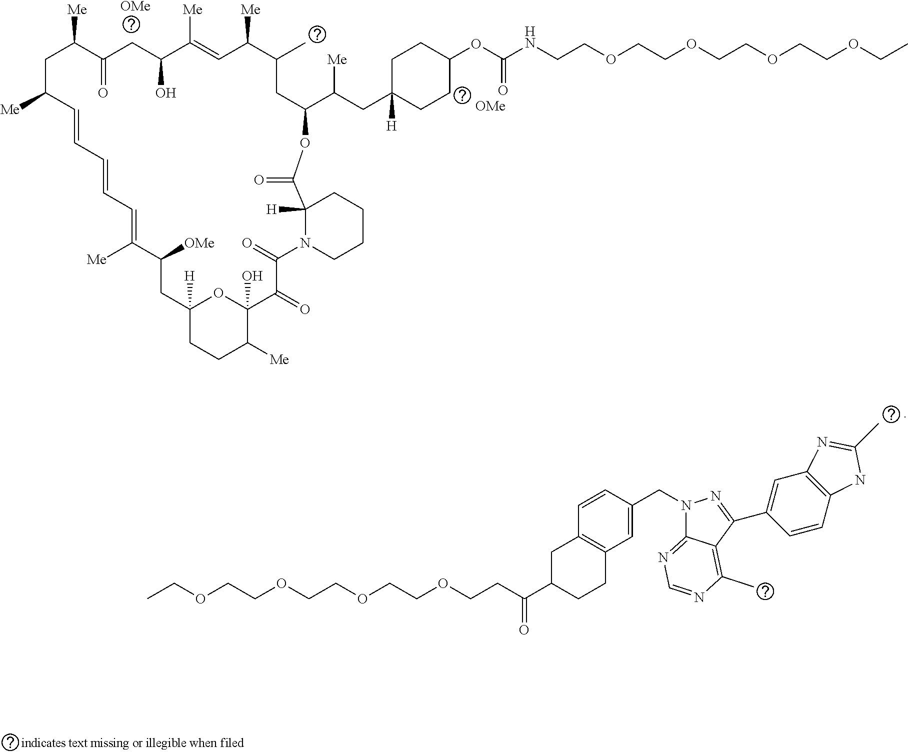

BRAF inhibitors that may be used in combination with compounds of the disclosure include, for example, vemurafenib, dabrafenib, and encorafenib. A BRAF may include a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581 I; D594N; D594G; D594A; D594H; F595L; G596D; G596R and A762E.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is a SHP2 inhibitor. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and PCT applications: WO 2022043685, WO 2022042331, WO 2022033430, WO 2022033430, WO 2022017444, WO 2022007869, WO 2021259077, WO 2021249449, WO 2021249057, WO 2021244659, WO 2021218755, WO 2021281752, WO 2021197542, WO 2021176072, WO 2021149817, WO 2021148010, WO 2021147879, WO 2021143823, WO 2021143701, WO 2021143680, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, US 20210085677, U.S. Ser. No. 10/858,359, U.S. Ser. No. 10/934,302, U.S. Ser. No. 10/954,243, U.S. Ser. No. 10/988,466, U.S. Ser. No. 11/001,561, U.S. Ser. No. 11/033,547, U.S. Ser. No. 11/034,705, U.S. Ser. No. 11/044,675, CN 114163457, CN 113896710, CN 113248521, CN 113248449, CN 113135924, CN 113024508, CN 112920131, CN 112823796, CN 111704611, CN 111265529, and CN 108113848, or a pharmaceutically acceptable salt, solvate, isomer (e.g., stereoisomer), prodrug, or tautomer thereof, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TNO155, having the structure

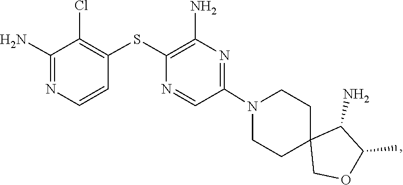 [SMILES: C[C@@H]1OCC2(CCN(c3cnc(Sc4ccnc(N)c4Cl)c(N)n3)CC2)[C@@H]1N]

or a pharmaceutically acceptable salt, solvate, isomer (e.g., stereoisomer), prodrug, or tautomer thereof.

In some embodiments, the SHP2 inhibitor is RMC-4550, having the structure

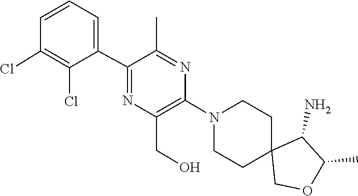 [SMILES: Cc1nc(N2CCC3(CC2)CO[C@@H](C)[C@H]3N)c(CO)nc1-c1cccc(Cl)c1Cl]

or a pharmaceutically acceptable salt, solvate, isomer (e.g., stereoisomer), prodrug, or tautomer thereof. In some embodiments, the SHP2 inhibitor is RMC-4630, having the structure

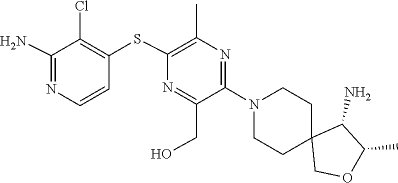 [SMILES: Cc1nc(N2CCC3(CC2)CO[C@@H](C)[C@H]3N)c(CO)nc1Sc1ccnc(N)c1Cl]

or a pharmaceutically acceptable salt, solvate, isomer (e.g., stereoisomer), prodrug, or tautomer thereof. In some embodiments, the SHP2 inhibitor is JAB-3068, having the structure

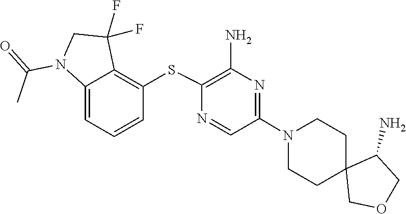 [SMILES: CC(=O)N1CC(F)(F)c2c(Sc3ncc(N4CCC5(CC4)COC[C@H]5N)nc3N)cccc21]

or a pharmaceutically acceptable salt, solvate, isomer (e.g., stereoisomer), prodrug, or tautomer thereof. In some embodiments, the SHP2 inhibitor is JAB-3312. In some ebodiments, the SHP2 inhibitor is the following compound,

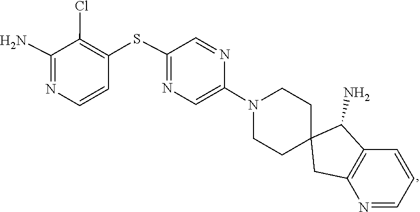 [SMILES: Nc1nccc(Sc2cnc(N3CCC4(CC3)Cc3ncccc3[C@H]4N)cn2)c1Cl]

or a pharmaceutically acceptable salt, solvate, isomer (e.g., stereoisomer), prodrug, or tautomer thereof. In some embodiments, the SHP2 inhibitor is RLY-1971, having the structure

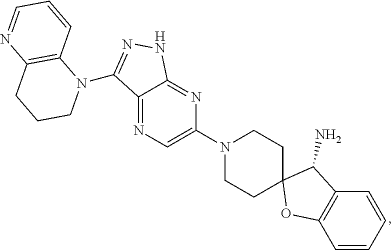 [SMILES: N[C@@H]1c2ccccc2OC12CCN(c1cnc3c(N4CCCc5ncccc54)n[nH]c3n1)CC2]

or a pharmaceutically acceptable salt, solvate, isomer (e.g., stereoisomer), prodrug, or tautomer thereof. In some embodiments, the SHP2 inhibitor is ERAS-601, or a pharmaceutically acceptable salt, solvate, isomer (e.g., stereoisomer), prodrug, or tautomer thereof. In some embodiments, the SHP2 inhibitor is BBP-398, or a pharmaceutically acceptable salt, solvate, isomer (e.g., stereoisomer), prodrug, or tautomer thereof. In some embodiments, the SHP2 inhibitor is SH3809. In some embodiments, the SHP2 inhibitor is PF-07284892, or a pharmaceutically acceptable salt, solvate, isomer (e.g., stereoisomer), prodrug, or tautomer thereof.

In some embodiments, the additional therapeutic agent is selected from the group consisting of a MEK inhibitor, a HER2 inhibitor, a SHP2 inhibitor, CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a RAS inhibitor of the present disclosure is used in combination with a MEK inhibitor and a SOS1 inhibitor. In some embodiments, a RAS inhibitor of the present disclosure is used in combination with a PDL-1 inhibitor and a SOS1 inhibitor. In some embodiments, a RAS inhibitor of the present disclosure is used in combination with a PDL-1 inhibitor and a SHP2 inhibitor. In some embodiments, a RAS inhibitor of the present disclosure is used in combination with a MEK inhibitor and a SHP2 inhibitor. In some embodiments, the cancer is colorectal cancer and the treatment includes administration of a RAS inhibitor of the present disclosure in combination with a second or third therapeutic agent.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PDL-1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the disclosure is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-(Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and Medlmmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProlX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the disclosure include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the disclosure is an autophagy inhibitor. Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the disclosure is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-la, interferon beta-Ib, interferon gamma, natural interferon gamma-la, interferon gamma-Ib, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the disclosure include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MED14736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-(Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the disclosure and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the disclosure and any of the therapies described herein are administered a few minutes apart, or a few hours apart, or a few days apart.

In some embodiments of any of the methods described herein, the first therapy (e.g., a compound of the disclosure) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The disclosure also features kits including (a) a pharmaceutical composition including an agent (e.g., one or more compounds of the disclosure) described herein, and (b) a package insert with instructions to perform any of the methods described herein. In some embodiments, the kit includes (a) a pharmaceutical composition including an agent (e.g., one or more compounds of the disclosure) described herein, (b) one or more additional therapies (e.g., non-drug treatment or therapeutic agent), and (c) a package insert with instructions to perform any of the methods described herein.

As one aspect of the present disclosure contemplates the treatment of the disease or symptoms associated therewith with a combination of pharmaceutically active compounds that may be administered separately, the disclosure further relates to combining separate pharmaceutical compositions in kit form. The kit may include two separate pharmaceutical compositions: a compound of the present disclosure, and one or more additional therapies. The kit may include a container for containing the separate compositions such as a divided bottle or a divided foil packet. Additional examples of containers include syringes, boxes, and bags. In some embodiments, the kit may include directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1. Heterogenous Acquired Resistance Alterations Converge on RAS-MAPK Reactivation

A 67-year-old female former light smoker was diagnosed with stage IV lung adenocarcinoma. Molecular testing of her primary lung tumor at initial diagnosis (‘pre-MRTX849 tissue’, 23.9 months prior to initiating MRTX849) revealed low-level PD-L1 expression (tumor proportion score of 20%, E1 L3N antibody) and KRAS G12C mutation, concomitant with STK11 splice region variant (c.734+5G>C), TP insertion/deletion (F338fs), RB1 splice region variant (c.1695+5_1695+15del), and FBXW7 loss. She was treated with first-line carboplatin, pemetrexed, pembrolizumab followed by maintenance pemetrexed and pembrolizumab for a total of approximately 15 months with stereotactic radiosurgery (SRS) to brain metastases, and then received a second-line investigational agent (an antibody drug conjugate) for 8.5 months before discontinuing for extracranial disease progression. The pre-MRTX849 cfDNA was collected prior to starting on the second-line investigational agent (18.2 months prior to initiating MRTX849).

The subject then enrolled in a dose expansion cohort of the phase 1 trial of adagrasib (MRTX849; KRYSTAL-1). She was treated with 600 mg twice daily dosing. The first restaging computed tomography (CT) after 6 weeks of treatment demonstrated a 32% reduction in tumor burden (per RECIST v1.1). Repeat imaging after 4 months of treatment showed progressive disease with increased right upper lobe lung mass, nodal metastases (axillary, anterior diaphragmatic, mediastinal, and internal mammary), and subcentimeter brain metastasis. She underwent biopsy of resistant plasma and SRS to the progressing brain lesion and continued to receive MRTX849 for clinical benefit. Six weeks later, CT scans confirmed further extracranial disease progression (FIG. 1A). Administration of MRTX849 was therefore discontinued. Serial plasma samples were collected at the time of MRTX849 discontinuation, 9 days post-discontinuation, and 51 days post-discontinuation (of note, the subject received 13 days of an investigational SHP2 inhibitor between the 9-day and 51-day timepoints).

In order to identify putative mechanisms of acquired resistance to MRTX849 in this subject, serial cell-free DNA (cfDNA) was assessed using a targeted next-generation sequencing assay (Guardant360, Guardant Health) and droplet digital PCR (ddPCR). Upon development of acquired resistance, the original KRAS^{G12C} and TP^{53F338fs} variants present in pre-treatment tumor and cfDNA were again detected in cfDNA, but were accompanied by the emergence of 10 distinct mutations affecting RAS-MAPK components KRAS, NRAS, BRAF, and MAP2K1 (which encodes the MEK1 protein) identified across cfDNA specimens obtained after disease progression (Table 1, Table 2).

TABLE 1
Variant allele fractions (VAFs) of mutations
detected in subject's serial plasma samples

| | | cfDNA | | | |
|---|---|---|---|---|---|
| | Tumor | | Days post-MRTX849 | | |
| | Pre- | Pre- | discontinuation: | | |
| | MRTX849 | MRTX849 | 0 | 6 | 48 |
| TP53 F338fs | 36.8% | 0.22% | 8.8% | 10.1% | 14.3% |
| KRAS G12C | 21.3% | 0.12% | 31.7% | 47.1% | 24.9% |
| KRAS G12V | — | — | — | — | 0.09% |
| KRAS G13D | — | — | — | 0.13%† | 0.04% |
| KRAS Y96D | — | — | 0.4% | 0.2% | — |
| NRAS Q61L | — | — | — | 0.2% | — |
| NRAS Q61R | — | — | — | — | 0.02% |
| NRAS Q61K | — | — | 0.6% | 0.6% | 0.9% |
| BRAF V600E | — | — | 0.1% | 0.1% | 0.5% |
| MAP2K1 | — | — | 0.05%† | — | 0.3% |
| K57N | | | | | |
| MAP2K1 | — | — | — | — | 0.1% |
| Q56P | | | | | |
| MAP2K1 | — | — | — | 0.12%† | 0.2% |
| E102_I103del | | | | | |

†indicates the mutations were detected by digital droplet PCR but not by plasma next generation sequencing (NGS)

TABLE 2
VAFs of KRAS G12C, G12V, G13D, and Y96D mutations
and MAP2K1 K57N and E102_I103del mutations

| Sample | Target | VAF % (pos/total events) | |
|---|---|---|---|
| Tumor tissue before | KRAS G12C | 31.4 | (463/1473) |
| MRTX849 | KRAS G12V | 0 | (0/1157) |
| | KRAS G13D | 0 | (0/1107) |
| | KRAS Y96D | 0 | (0/1439) |
| | MAP2K1 K57N | 0 | (0/959) |
| | MAP2K1 | 0 | (0/830) |
| | E102_I103del | | |
| 0 days post- | KRAS G12C | 31.3 | (600/1920) |
| MRTX849 | KRAS G12V | 0 | (0/2254) |
| discontinuation | KRAS G13D | 0 | (0/456) |
| | KRAS Y96D | 0.19 | (1/507) |
| | MAP2K1 K57N | 0.05 | (1/1820) |
| | MAP2K1 | 0 | (0/378) |
| | E102_I103del | | |
| 9 days post- | KRAS G12C | 47.8 | (1694/3543) |
| MRTX849 | KRAS G12V | 0 | (0/3690) |
| discontinuation | KRAS G13D | 0.13 | (1/757) |
| | KRAS Y96D | 0.14 | (2/1350) |
| | MAP2K1 K57N | 0 | (0/2729) |
| | MAP2K1 | 0.12 | (1/789) |
| | E102_I103del | | |
| 51 days post- | KRAS G12C | 21 | (443/2109) |
| MRTX849 | KRAS G12V | 0.09 | (1/1083) |
| discontinuation | KRAS G13D | 0.1 | (1/987) |
| | KRAS Y96D | 0 | (0/948) |
| | MAP2K1 K57N | 0.13 | (1/744) |
| | MAP2K1 | 0.5 | (2/372) |
| | E102_I103del | | |

pos = positive.

The lower allele frequencies of these alterations relative to the truncal KRAS^{G12C} and TP mutations are consistent with the emergence of these mutations in heterogeneous subclonal populations. These included three activating NRAS mutations (NRAS^{Q61L}, NRAS^{Q61K}, NRAS^{Q61R}), which can drive active RAS signaling in a KRAS-independent manner, and BRAF^{V600E}, which can maintain MAPK signaling downstream of KRAS^{G12C} in the presence of MRTX849 (FIG. 1B). Three MAP2K1 mutations (MAP2K1^{K57N}, MAP2K1^{Q56R}, MAP2K1^{E102-1103del}) previously demonstrated to be activating and known to be involved in resistance to upstream MAPK pathway inhibitors (i.e. BRAF inhibitors) were also identified (see Kinosh ita-Kikuta et al. Biochim Biophys Acta Proteins Proteom 1867(1):62-70 (2019) and Gao et al. Cancer Discov 8(5):648-661 (2018), which are incorporated herein by reference).

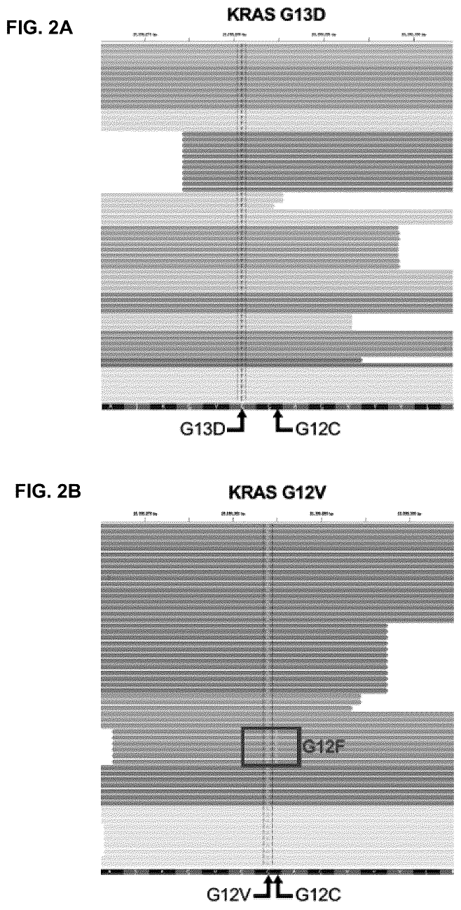

Additionally, three KRAS mutations emerged in the post-progression cfDNA. Two of these mutations are known activating mutations KRAS^{G13D} and KRAS^{G12V}, and mutant-selective KRAS^{G12C} inhibitors have previously been shown to be ineffective against these mutations (see Hallin et al. Cancer Discov 10(1):54-71 (2020) and Canon et al. Nature 575(7781):217-223 (2019), which are incorporated herein by reference). A deeper analysis of individual sequencing reads from cfDNA suggested that these mutations seemed to occur in trans to the original KRAS^{G12C} mutation (FIG. 2A, FIG. 2B), likely arising in the remaining wild type copy of KRAS, which appeared to be retained based on pre-treatment tumor sequencing (Table 3).

TABLE 3
Copy number of KRAS in the analyzed tumor and
cell-free DNA samples pre- and post-MRTX849

| | | cfDNA | | | |
|---|---|---|---|---|---|
| | Tumor | | Days post-MRTX849 | | |
| | Pre- | Pre- | discontinuation: | | |
| | MRTX849 | MRTX849 | 0 | 9 | 51 |
| KRAS copy # | 2% | NA | 2.71% | 3.67% | 2.3% |

#, number;
NA = not assessable due to low tumor fraction

However, it is not possible from the cfDNA data to confirm that these mutations co-exist in cells that also harbor the original KRAS^{G12C} mutation. Notably, a single, well-supported family of sequencing reads from the same original template molecule showed the concurrent presence of both nucleotide changes corresponding to KRAS^{G12C} and KRAS^{G12V} in cis on the same strand, which would encode for a KRAS^{G12F} mutation. While it is not possible to confirm the presence of this mutation based on a single read family, this finding raises the possibility that cis mutations resulting in “loss” of the original KRAS^{G12C} mutation and conversion to a different KRAS mutation might be another potential mechanism of resistance. Notably, all putative resistance mutations identified are predicted to converge on reactivation of RAS-MAPK pathway signaling, suggesting that this may represent a common primary mechanism of acquired resistance to KRAS^{G12C} inhibitors (FIG. 1C).

Interestingly, the third KRAS mutation identified, KRAS^{Y96D}, represents a novel mutation that is not known to be activating. Notably, while KRAS is the most commonly mutated oncogene in human cancer, a search of two large tumor mutational databases-COSMIC and GENIE, which collectively contain >450,000 molecularly characterized cancers (see Sondka et al. Nat Rev Cancer 18(11):696-(2018) and Consortium APG. Cancer Discov 7(8):818-831 (2017), which are incorporated herein by reference)—did not reveal a single previously identified mutation at the KRAS^{Y96} locus among >75,000 cases with documented KRAS mutations (Table 4). However, the Y96 residue is associated with the Switch-II pocket to which MRTX849 and other inactive-state KRAS^{G12C} inhibitors bind, suggesting that the previously undescribed Y96D mutation may have a novel and specific role in driving resistance to KRAS^{G12C} inhibitors.

TABLE 4
Frequencies of all KRAS mutations,
KRAS^{G12C}, and KRAS^{Y96D} according to COSMIC
v93 and AACR Project GENIE (cBioPortal) databases

| | Mutations (% of total) | | | | | |
|---|---|---|---|---|---|---|
| | | | | | | Total |
| | | | | | | Samples |
| Database | KRAS | | KRAS^{G12C} | | KRAS^{Y96D} | Tested |
| COSMIC v92 | 47,339 | (17.9) | 5,426 | (2) | 0 | 264,108 |
| AACR | 29,046 | (16) | 4,249 | (2.3) | 0 | 186,433 |
| Project | | | | | | |
| GENIE | | | | | | |
| (cBioPortal) | | | | | | |
| v.3.6.6(1, 2) | | | | | | |

### Example 2. Structural Modeling of KRAS^{G12C/Y96D}

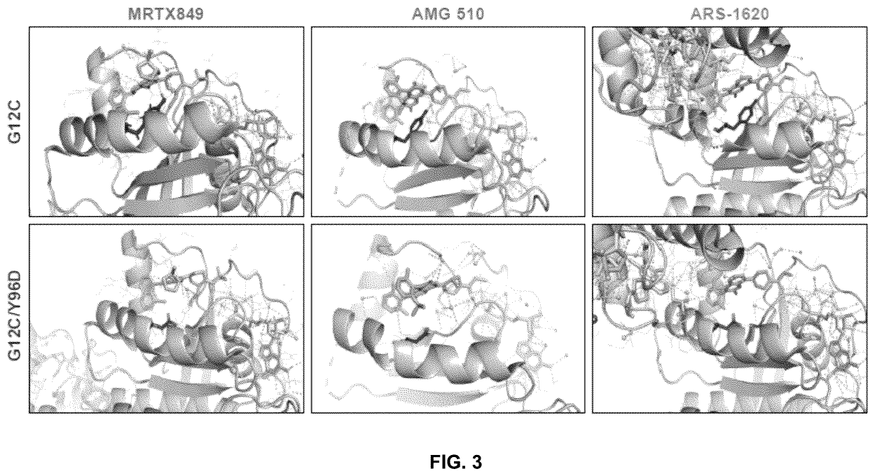

To understand the significance of the acquired KRAS^{Y96D} mutation, structural modeling of the G12C-mutant and G12C/Y96D-double mutant KRAS proteins bound to the KRAS^{G12C} inhibitors MRTX849, AMG 510, and ARS-1620 was performed (FIG. 3). These three inhibitors bind the GDP-state of KRAS^{G12C} and exploit a cryptic pocket formed by the central beta sheet of RAS and Switch II (first identified by Ostrem et al. (Nature 503(7477):548-551 (2013), which is incorporated herein by reference). To determine the effects of the amino acid substitution at the Y96 locus, crystal structures of MRTX849, AMG 510, and ARS-1620 bound to KRAS^{G12C} were modeled for interactions with the Y96 residue within the Switch II pocket (see Canon et al. Nature 575(7781):217-223 (2019), Fell et al. ACS Med Chem Lett 9(12):1240-1234 (2018), Janes et al. Cell 172(3):578-589 (2018), and Chen et al. J Med Chem 63(23):14404-14424 (2020), which are incorporated herein by reference). The hydroxyl group of Y forms a direct hydrogen bond with the pyrimidine ring of MTRX849, which is abolished with the Y96D mutation. Y96D also disrupts the water mediated hydrogen bond between Y96 and a carboxyl group on AMG 510. Finally, while Y96 does not form a direct hydrogen bond with ARS-1620, it stabilizes the interaction with ARS-1620 through pi-stacking with the phenyl ring of Y96, which is disrupted with the Y96D mutation. Additionally, by introducing a negatively charged amino acid, the Y96D mutation changes the hydrophobic nature of the binding pocket for all three compounds to a substantially more hydrophilic pocket, which may further destabilize binding.

### Example 3. Functional Characterization of KRAS^{Y96D}

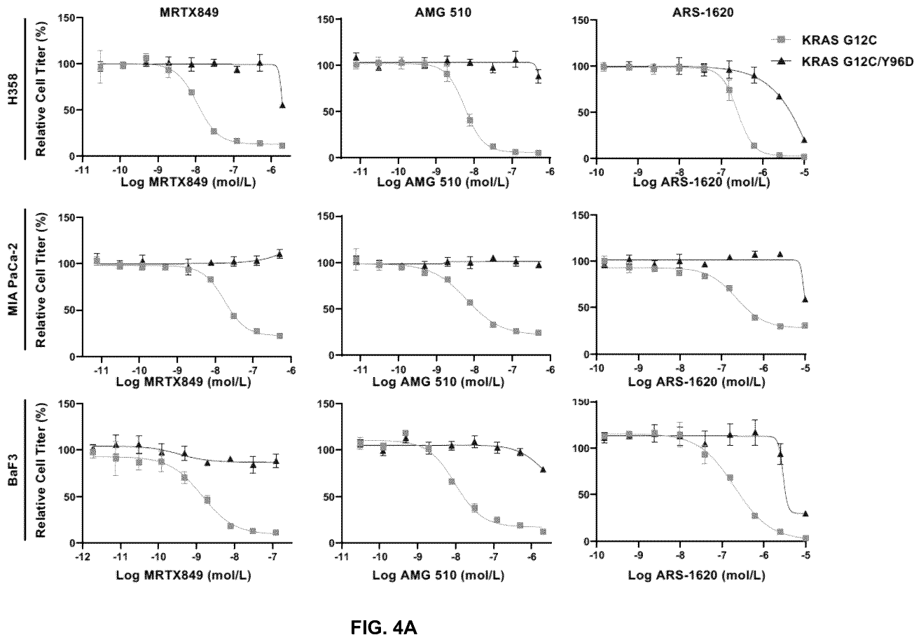

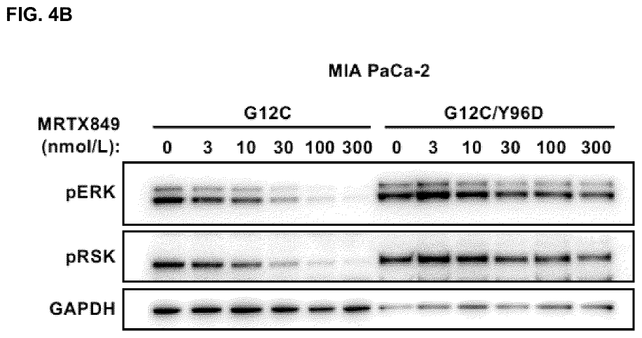

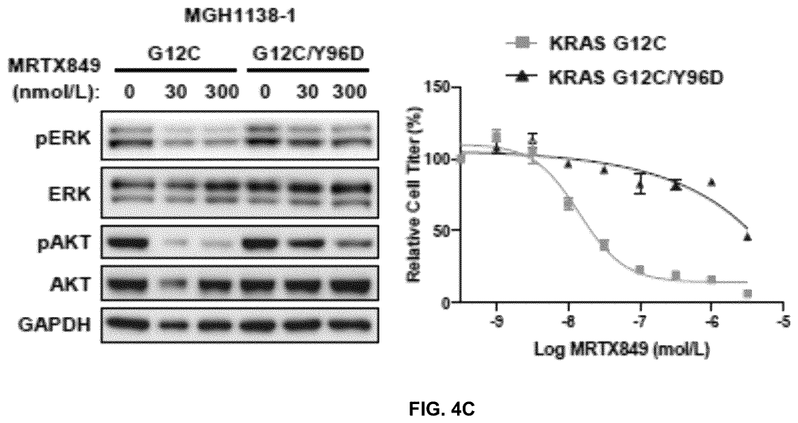

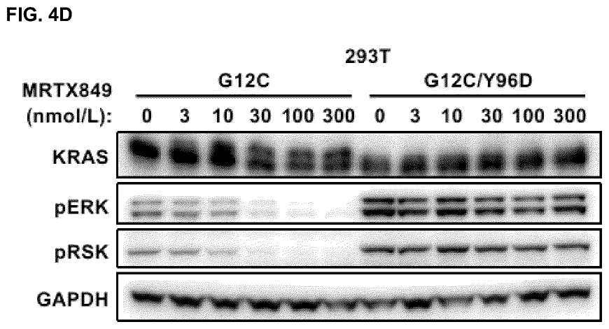

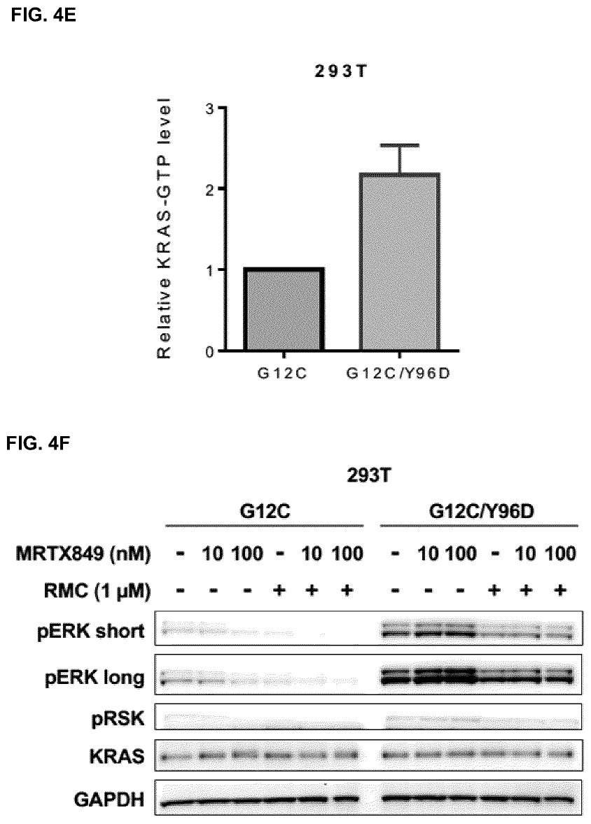

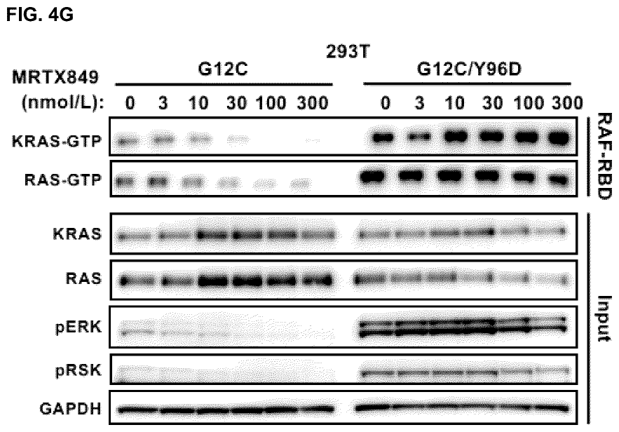

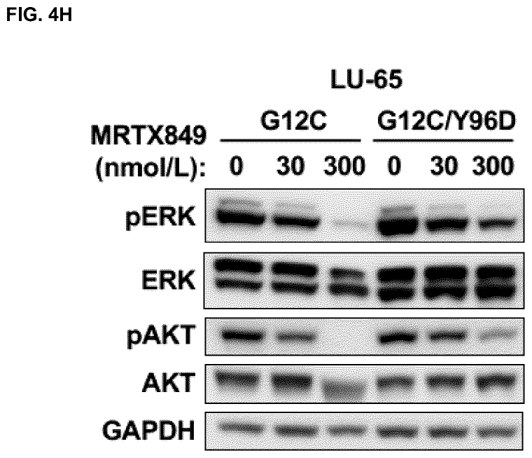

To assess whether KRAS^{Y96D} can mediate resistance to MRTX849 and other inactive-state KRAS^{G12C} inhibitors, KRAS^{G12C} or the KRAS^{G12C/Y96D} double mutant in NCI-H358 (KRAS^{G12C}-mutant NSCLC), MIA PaCa-2 (KRAS^{G12C}-mutant pancreatic ductal adenocarcinoma), as well as Ba/F3 cells, which lack endogenous KRAS^{G12C}, but become oncogene-dependent upon withdrawal of IL-3, were expressed. In cell viability assays, relative to KRAS^{G12C}-expressing controls, cells expressing KRAS^{G12C/Y96D} showed marked resistance to three KRAS^{G12C} inhibitors, with IC₅₀ shifts of >100-fold for MRTX849 and AMG 510 and ˜20-fold for ARS-1620 (FIG. 4A, Table 5).

TABLE 5
The IC₅₀ values of KRAS inhibitors in cell lines expressing KRAS^{G12C} or KRAS^{G12C/Y96D}
IC₅₀ (mol/L)

| Cell | MRTX849 | | AMG 510 | | ARS-1620 | | RM-018 | |
|---|---|---|---|---|---|---|---|---|
| line | G12C | G12C/Y96D | G12C | G12C/Y96D | G12C | G12C/Y96D | G12C | G12C/Y96D |
| H358 | 2.2 × 10⁻⁸ | >2.0 × 10⁻⁶ | 6.1 × 10⁻⁹ | >2.0 × 10⁻⁶ | 2.6 × 10⁻⁷ | 3.8 × 10⁻⁶ | 3.5 × 10⁻⁹ | 7.3 × 10⁻⁹ |
| MIA | 2.1 × 10⁻⁸ | >2.0 × 10⁻⁶ | 6.9 × 10⁻⁹ | >2.0 × 10⁻⁶ | 1.4 × 10⁻⁷ | 9.4 × 10⁻⁶ | 1.4 × 10⁻⁹ | 3.4 × 10⁻⁹ |
| PaCa-2 | | | | | | | | |
| Ba/F3 | 1.4 × 10⁻⁸ | >2.0 × 10⁻⁶ | 8.6 × 10⁻⁹ | >2.0 × 10⁻⁶ | 2.1 × 10⁻⁷ | 3.0 × 10⁻⁶ | 1.4 × 10⁻⁹ | 2.8 × 10⁻⁹ |

Consistent with the effects on cell viability, RAS-MAPK pathway activity, as measured by levels of phosphorylated (p)ERK and pRSK were sustained in KRAS^{G12C/Y96D}-expressing MIA PaCa-2 cells even at high concentrations of MRTX849, relative to cells expressing KRAS^{G12C} alone (FIG. 4B). Similarly, in KRAS^{G12C}-mutant non-small cell lung cancer (NSCLC) cells in which PI3K signaling is driven by mutant KRAS, including an existing patient-derived model MGH1138-1, persistent pERK and pAKT levels were observed with KRAS^{G12C/Y96D} in the presence of MRTX849, relative to KRAS^{G12C} expression alone (see FIG. 4C for MGH1138-1 cells; FIG. 4H for LU-65 cells). KRAS^{G12C/Y96D} also drove marked resistance to MRTX849 in the patient-derived MGH1138-1 model. Furthermore, in 293T cells, which lack endogenous KRAS^{G12C} expression, MRTX849 was unable to inhibit pERK levels driven by KRAS^{G12C/Y96D} (FIG. 4D). Since MRTX849 and other inactive-state KRAS^{G12C} inhibitors bind covalently to KRAS^{G12C}, an electrophoretic mobility shift of drug-adducted KRAS^{G12C} can be observed upon drug binding due to increased molecular weight. However, this mobility shift was no longer observed when 293T cells expressing KRAS^{G12C/Y96D} were treated with MRTX849, suggesting that the Y96D mutation may abrogate inhibitor binding. Notably, KRAS^{G12C/Y96D} appeared to have higher basal activation than KRAS^{G12C}, as measured by a higher proportion of the active GTP-bound form of KRAS, though activation still appeared to be partly dependent on upstream pathway input (FIG. 4E, FIG. 4F). Finally, while a decrease in guanosine triphosphate (GTP)-bound KRAS (representing the active state) was observed in KRAS^{G12C}-expressing cells treated with MRTX849, levels of active GTP-bound KRAS were maintained in KRAS^{G12C/Y96D} expressing cells (FIG. 4G) (see Zeng et al. Cell Chem Biol 24(8):1005-1016 (2017), which is incorporated herein by reference). These results suggest that the KRAS^{Y96D} mutation disrupts KRAS^{G12C} inhibitor binding, leading to sustained KRAS signaling and therapeutic resistance.

### Example 4. An active state KRAS^{G12C} inhibitor overcomes KRAS^{G12C/Y96D}

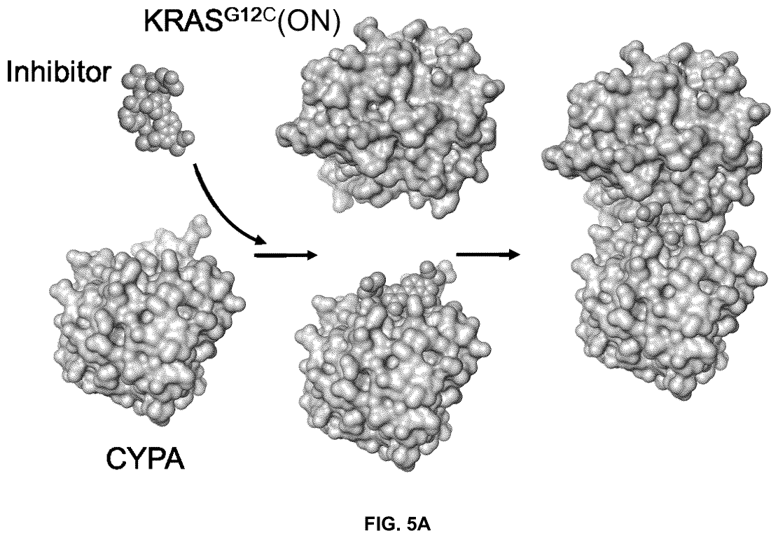

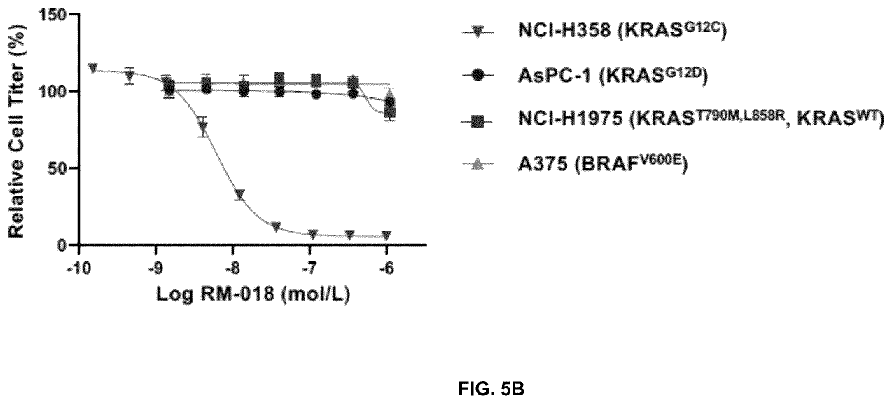

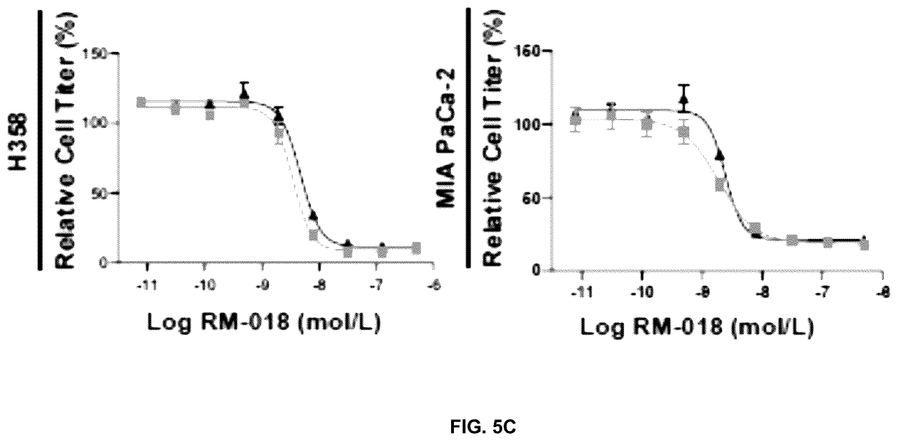

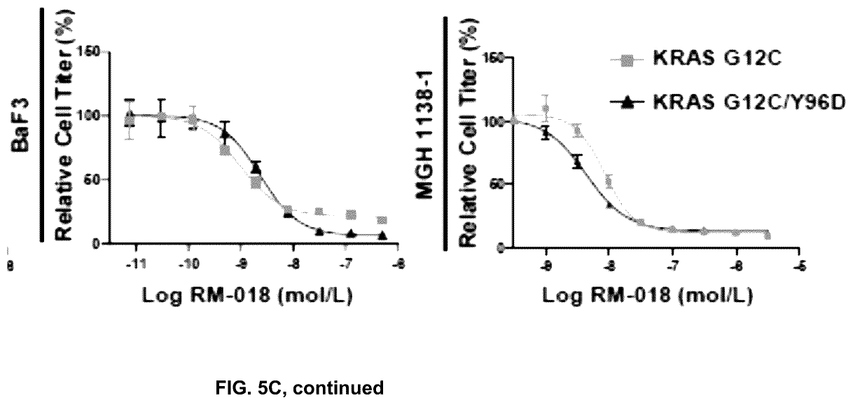

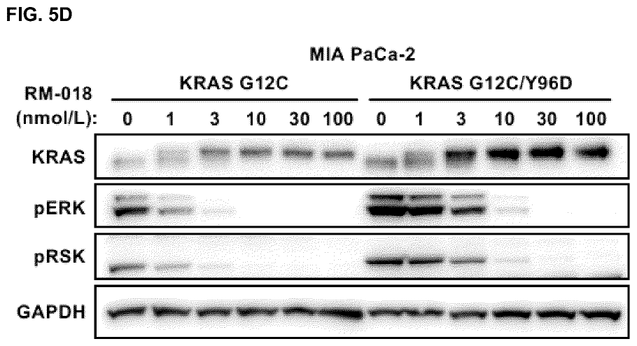

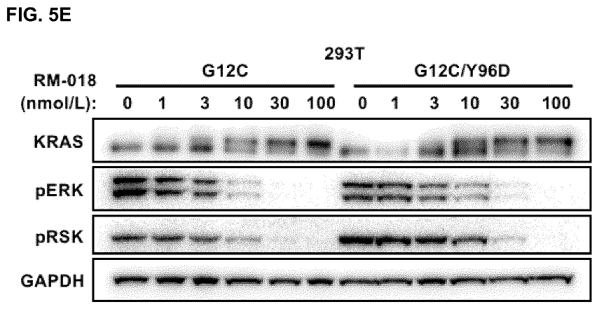

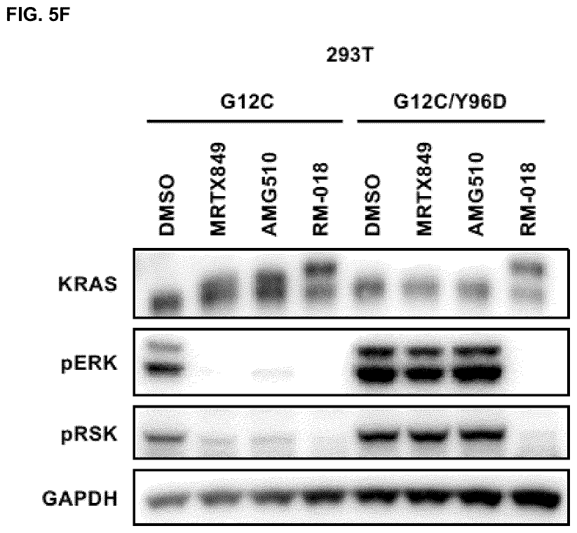

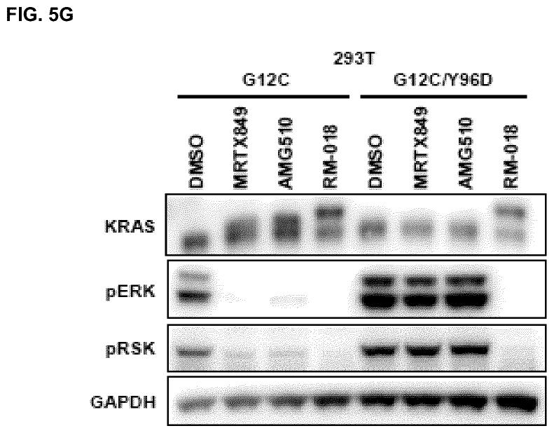

As KRAS^{G12C/Y96D} conferred resistance to multiple KRAS^{G12C} inhibitors currently in clinical development, suggestive of shared vulnerability for this class of inhibitors, it was important to identify whether a structurally and functionally distinct KRAS^{G12C} inhibitor might retain potency against this resistance mutation. RM-018 (a KRAS^{G12C} inhibitor which binds specifically to the GTP-bound, active (“RAS(ON)”) state of KRAS^{G12C}) is a “tri-complex” KRAS inhibitor, which exploits a highly abundant chaperone protein, cyclophilin A, to bind and inhibit KRAS^{G12C}, as previously described (FIG. 5A) (Schulze et al. Molecular Cancer Therapeutics 18(12 Supplement):PR10-PR (2019) and Nichols et al. Journal of Thoracic Oncology 15(2):S13-S14 (2020), which are incorporated herein by reference). Briefly, upon entering the cell, forms a “binary complex” with cyclophilin A. This binary complex can associate with the active state of KRAS^{G12C}, aided by protein-protein surface interactions between cyclophilin A and KRAS, and forms a covalent bond with KRAS^{G12C} in a mutant-selective manner. This resultant “tri-complex” inhibits KRAS^{G12C} through binding of cyclophilin A leading to steric occlusion preventing association of downstream effector proteins. Given the markedly different mechanism of action of this class of inhibitor, we hypothesized that the inhibitory activity of RM-018 might be differentially affected by KRAS^{Y96D} compared to inactive-state KRAS^{G12C} inhibitors.

RM-018 demonstrated selectivity for KRAS^{G12C}-driven cells, exhibiting low nanomolar potency in KRAS^{G12C}-mutant H358 cells, while not impairing the viability of cells driven by KRAS^{G12D}, BRAF^{V600E} or RTK-driven signaling through wild-type RAS (FIG. 5B). Interestingly, while KRAS^{G12C/Y96D} expression led to marked IC₅₀ shifts of >100-fold for MRTX849 and AMG 510 and ˜20-fold for ARS-1620 (FIG. 4A) relative to KRAS^{G12C} expression alone, the efficacy of RM-018 on cell viability was largely unaffected by KRAS^{G12C/Y96D} expression with IC₅₀ shifts of only ˜2-fold (FIG. 5C, Table 5). In addition, RM-018 was able to inhibit pERK and pRSK levels with similar potency in the presence of KRAS^{G12C} or KRAS^{G12C/Y96D} expression in both MIA PaCa-2, 293T cells, and the patient-derived KRAS^{G12C}-mutant NSCLC cell line MGH1138-1 (FIG. 5D, FIG. 5E, FIG. 5F). Inhibition of cell viability by RM-018 was also unaffected by KRAS^{G12C/Y96D} expression in the patient-derived MGH1138-1 model. Furthermore, the KRAS mobility shift induced by covalent binding of RM-018 was observed in both cell lines in the presence of either KRAS^{G12C} or KRAS^{G12C/Y96D} expression, suggesting that binding of RM-018 to KRAS is not abrogated by the KRAS^{Y96D} mutation. Indeed, while a KRAS mobility shift due to covalent drug binding was observed in 293T cells expressing KRAS^{G12C} for MRTX849, AMG 510, and RM-018, only RM-018 exhibited this same mobility shift and was able to inhibit downstream signaling in the presence of the KRAS^{G12C/Y96D} mutation (FIG. 5G). Taken together, these data suggest that RM-018 retains the ability to bind and inhibit KRAS^{G12C/Y96D} and may represent a potential therapeutic strategy to overcome this acquired resistance mechanism.

### Example 5. Treatment of a Cancer Having a RAS Mutation in a Subject

The methods of the disclosure can be used to treat a cancer (e.g., a cancer described herein (e.g., non-small cell lung cancer) that has or has not been treated with a RAS(OFF) inhibitor (e.g., MRTX849, AMG 510, ARS-1620) characterized by a RAS mutation (e.g., a RAS mutation described herein, such as, e.g., a G12C or a Y96D substitution) in a human subject. Optionally, a sample (e.g., a plasma sample) may be taken from the subject to determine the variant allele fraction of mutations. The subject may be administered a therapeutically effective amount of a RAS(ON) inhibitor described herein.

The RAS(ON) inhibitor may be administered after a RAS(OFF) inhibitor has been administered (e.g., in the event the cancer becomes resistant to the RAS(OFF) inhibitor or the cancer progresses when the RAS(OFF) inhibitor is administered to the subject). Optionally, the RAS(ON) inhibitor may be administered with a RAS(OFF) inhibitor (e.g., simultaneously or sequentially). Simultaneous administration of the RAS(ON) and RAS(OFF) inhibitors could be, e.g., a single formulation or separate formulation. Sequential administration of the RAS(ON) and RAS(OFF) inhibitors could involve, e.g., administering the RAS(OFF) inhibitor for a first period of time then administering the RAS(ON) inhibitor for a second period of time, where the first period of time and the second period of time do not overlap (and the first period of time precedes the second period of time). Optionally, sequential administration of the RAS(ON) and RAS(OFF) inhibitors could involve, e.g., administering the RAS(OFF) inhibitor for a first period of time then administering the RAS(OFF) and RAS(ON) inhibitor for a second period of time, where the first period of time and the second period of time do not overlap (and the first period of time precedes the second period of time).

The progression of the cancer that is treated with the RAS(ON) inhibitor can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with a RAS(ON) inhibitor. Based on the results of these analyses, a physician may prescribe higher/lower dosages or more/less frequent dosing of the RAS(ON) inhibitor in subsequent rounds of treatment.

### Example 6. Treatment of a Cancer Having a RAS Mutation at Residue Y96 in a Subject

The methods of the disclosure can be used to treat a cancer (e.g., a cancer described herein (e.g., non-small cell lung cancer) that has or has not been treated with a RAS(OFF) inhibitor (e.g., MRTX849, AMG 510, ARS-1620) characterized by a RAS mutation at residue Y96 (e.g., a Y96D substitution) in a human subject. A sample (e.g., a plasma sample) may be taken from the subject to determine the variant allele fraction of mutations. If a Y96 mutation in RAS is present, the subject may be administered a therapeutically effective amount of a RAS(ON) inhibitor described herein.

The RAS(ON) inhibitor may be administered after a RAS(OFF) inhibitor has been administered (e.g., in the event the cancer becomes resistant to the RAS(OFF) inhibitor or the cancer progresses when the RAS(OFF) inhibitor is administered to the subject). Optionally, the RAS(ON) inhibitor may be administered with a RAS(OFF) inhibitor (e.g., simultaneously or sequentially). Simultaneous administration of the RAS(ON) and RAS(OFF) inhibitors could be, e.g., a single formulation or separate formulation. Sequential administration of the RAS(ON) and RAS(OFF) inhibitors could involve, e.g., administering the RAS(OFF) inhibitor for a first period of time then administering the RAS(ON) inhibitor for a second period of time, where the first period of time and the second period of time do not overlap (and the first period of time precedes the second period of time). Optionally, sequential administration of the RAS(ON) and RAS(OFF) inhibitors could involve, e.g., administering the RAS(OFF) inhibitor for a first period of time then administering the RAS(OFF) and RAS(ON) inhibitor for a second period of time, where the first period of time and the second period of time do not overlap (and the first period of time precedes the second period of time).

The progression of the cancer that is treated with the RAS(ON) inhibitor can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with a RAS(ON) inhibitor. Based on the results of these analyses, a physician may prescribe higher/lower dosages or more/less frequent dosing of the RAS(ON) inhibitor in subsequent rounds of treatment.

### Example 7. Methods

### Subject Treatment and Specimen Collection

The subject was treated with MRTX849 dosed 600 mg twice daily on the phase 1 study (KRYSTAL-1) after providing written informed consent (ClinicalTrials.gov identifier: NCT03785249). She had received two prior lines of therapy. All pre- and post-treatment biopsies and genotyping were performed in accordance with the Massachusetts General Hospital (MGH) institutional review board-approved protocol and in accordance with the Declaration of Helsinki. The pre-treatment tumor specimen was analyzed using the MGH SNaPshot next-generation sequencing assay (see Zheng et al. Nat Med 20(12):1479-84 (2014), which is incorporated herein by reference). All cfDNA samples were sequenced using the commercially available Guardant360 assay (Guardant Health; Redwood City, CA).

### Cell Lines and Reagents

Ba/F3 cells were obtained from the RIKEN BRC Cell Bank (RIKEN BioResource Center). MGH1138-1 cells were generated from a KRAS^{G12C}-mutant NSCLC subject using methods that have been previously described (see Crystal et al. Science 346(6216):1480-1486 (2014), which is incorporated herein by reference). Prior to cell line generation, the subject provided written informed consent to participate in a Dana Farber/Harvard Cancer Center Institutional Review Board-approved protocol giving permission for research to be performed on their sample. The remaining cell lines were obtained from ATCC or the Center for Molecular Therapeutics at the MGH Cancer Center (Boston, MA) which routinely performs cell line authentication testing by SNP and short-tandem repeat analysis. HEK293T cells were maintained in DMEM supplemented with 10% FBS. MIA PaCa-2 and NCI-H358 cells were maintained in DMEM/F12 supplemented with 10% FBS. LU-65 and MGH1138-1 cells were maintained in RPMI supplemented with 10% FBS. Ba/F3 cells were maintained in DMEM supplemented with 10% FBS and 10 ng/mL interleukin-3 (IL-3). KRAS (G12C or G12C/Y96D) gene was inserted in pMXs-Puro Retroviral Expression Vector, which was purchased from Cell Biolabs. Retrovirus packaging mutated KRAS genes were produced with HEK293T cells. After concentration of virus with Retro-Concentin Retro Concentration Reagent (System Biosciences), MIA PaCa-2, NCI-H358 and Ba/F3 cells were infected with the virus packaging either KRAS G12C or G12C/Y96D gene. After 48 hours of incubation, the cells were treated with puromycin (1-2 μg/mL) for another 48 hours. IL-3 was withdrawn to select for Ba/F3 cells dependent on mutant KRAS signaling after 48 hours of puromycin treatment. The remaining cells were maintained in media supplemented with puromycin. For transient expression experiments, a day after seeding the cells, pMXs-Puro-KRAS^{G12C} or pMXs-Puro-KRAS^{G12C/Y96D} vectors were induced with Lipofectamine 2000 Transfection Reagent (ThermoFisher Scientific) following manufacturer's protocol. After 16-24 hours of incubation, cells were treated with inhibitors for 4 hours. AMG 510 was purchased from MedChemExpress. MRTX849 and ARS-1620 were purchased from Selleck Chemicals. RM-018 was provided by Revolution Medicines (Redwood City, CA, USA), and details of the chemical synthesis of RM-018 can be found in Appendix B, and in International Patent Application No. PCT/US2020/058841, which is incorporated by reference in its entirety. RM-018 may also be prepared using methodologies known to those of skill in the art.

### Cell Viability Assays

Cells lines were seeded in 96-well plate at 2-10×10³ cells/well depending on cell lines and after 24 treated with a serial dilution of drugs and incubated for 72 hours. Cell viability was measured with CellTiter-Glo (Promega).

### Western Blot Analysis

Cell lines were treated with MRTX849, AMG 510 or RM-018 for 4 hours and lysates were prepared as described by Ahronian et al. Cancer Discov 5(4):458-67 (2015), which is incorporated herein by reference. All antibodies were diluted in 5% bovine serum albumin as follows: KRAS (Sigma), phospho-ERK (Thr202/Tyr204,1:1,000, Cell Signaling Technology), p44/42 MAPK (Erk1/2) (1:1000, Cell signaling Technology), phospho-RSK1 (T359+S363, 1:1,000, Abcam), phospho-Akt (Ser473, 1:1000, Cell Signaling Technology), AKT (1:1000, Cell Signaling Technology) and GAPDH (1:1,000, MilliporeSigma).

### RAS-GTP Pulldown

After indicated inhibitor treatment, RAS activity was assessed by GST-RAF-RBD pulldown (Cell Signaling Technology), followed by western blot analysis with pan-RAS or RAS isoform-specific antibodies. Pulldown samples and whole-cell lysates were resolved on 4-12% Bis-Tris Gels and western blotting was performed using antibodies against KRAS (Sigma) and pan-RAS (Cell Signaling Technology).

### Structural Modeling

Publicly available crystal structures of KRAS^{G12C} in complex with MRTX849 (PDB:6UT0), AMG (PDB:601M), and ARS-1620 (PDB:5V9U) were downloaded from the RCSB Protein Data Bank (PDB) (see Berman et al. Nucleic Acids Research 28(1):235-242 (2000), which is incorporated herein by reference). Structures were rendered in PyMol (The PyMOL Molecular Graphics System) and analyzed for hydrogen bonds and other molecular interactions between the KRAS^{G12C} inhibitors and the KRAS protein. Structures of Y96 amino acid mutation were generated by Protein Mutagenesis Wizard implemented in PyMol, with one of the backbone dependent rotamers manually selected.

ctDNA Extraction and Digital Droplet PCR

Whole blood was collected by routine phlebotomy in two 10 mL Streck tubes. Plasma was separated within 1-4 days of collection through two different centrifugation steps (the first at room temperature for 10 minutes at 1,600×g and the second at 3,000×g for the same time and temperature). Plasma was stored at −80° C. until ctDNA extraction. ctDNA was extracted from plasma using the QIAamp Circulating Nucleic Acid Kit (QIAGEN) with 60 min of proteinase K incubation at 60 degrees Celsius. All other steps were performed according to the manufacturer's instructions. For droplet digital PCR (ddPCR) experiments, DNA template (up to 10 μL, with a total of 20 ng) was added to 12.5 μL of ddPCR Supermix for Probes (Bio-Rad) and 1.25 μL of the custom primer/probe mixture. This reaction mix was added to a DG8 cartridge together with 60 μL of Droplet Generation Oil for Probes (Bio-Rad) and used for droplet generation. Droplets were then transferred to a 96-well plate (Eppendorf) and then thermal cycled with the following conditions: 5 minutes at 95° C., 40 cycles of 94° C. for 30 seconds, 55° C. (with a few grades difference among assays) for 1 minute followed by 98° C. for 10 minutes (Ramp Rate 2° C./sec). Droplets were analyzed with the QX200 Droplet Reader (Bio-Rad) for fluorescent measurement of FAM and HEX probes. Gating was performed based on positive and negative controls, and mutant populations were identified. The ddPCR data were analyzed with QuantaSoft analysis software (Bio-Rad) to obtain Fractional Abundance of the mutant DNA alleles in the wild-type/normal background. The quantification of the target molecule was presented as the number of total copies (mutant plus wild-type) per sample in each reaction. Allelic fraction is calculated as follows: AF %=(Nₘᵤₜ/(Nₘᵤₜ+N_{wt}))*100), where Nₘᵤₜ is the number of mutant alleles and N_{wt} is the number of wild-type alleles per reaction. ddPCR analysis of normal control plasma DNA (from cell lines) and no DNA template controls were always included. Probe and primer sequences are available upon request.

### Example 8. Compound AA, a KRAS^{G12C}(ON) inhibitor, inhibits KRAS^{G12C/Y96D} in cells

The MIAPaCa-2 cell line (homozygous for KRAS^{G12C}) was genetically modified to introduce the Y96D mutation into at least one KRAS allele. These cells were plated in 96-well tissue culture plates at 2500 cells/well in complete growth media (DMEM+10% FBS+1% PenStrep) and incubated overnight. Compounds were added at the indicated concentration and the plates were incubated for 5 days. Cellular viability was measured using Promega CellTiter-Glo 2.0 reagent according to manufacturer's instructions. The cellular viability signal for each well was normalized to 0.1% DMSO controls.

### Example 9. pERK Potency of Compound AA, a KRAS^{G12C}(ON) Inhibitor, in KRAS^{G12C/Y96D} Cells

The MIAPaCa-2 cell line (homozygous for KRAS^{G12C}) was genetically modified to introduce the Y96D mutation into at least one KRAS allele. These cells were plated in 96-well tissue culture plates at 30000 cells/well in complete growth media (DMEM+10% FBS+1% PenStrep) and incubated overnight. Compounds were added at the indicated concentration and the plates were incubated for 4 hours. Levels of phosphorylated and total ERK1/2 were assessed by Meso Scale Discovery assay kit. The ratio of phosphorylated ERK1/2 signal to total ERK1/2 signal for each well was normalized to the 0.1% DMSO controls.

### Example 10. Synthesis of Compounds of Table A1

Compounds of Table A1, and intermediates in the synthesis thereto, were prepared according to experimental procedures detailed in the Example section of WO 2021/091956, which is incorporated herein by reference in its entirety.

### Example 11. Biological Assay Data for Compounds of Table A1

Compounds of Table A1 were tested in the following biological assays as described in detail in WO 2021/091956: decrease in cellular pERK; determination of cell viability in RAS mutant cancer cell lines; disruption of B-Raf Ras-binding domain (BRAFRBD) interaction with K-Ras; and in vivo pharmacodynamic and efficacy.

The corresponding data for compounds of Table A1 evaluated in the assays described above are given in Tables 4-20, FIG. 1A, FIG. 1B, and the Examples section of WO 2021/091956.

### Example 12. Synthesis of Compounds of Table B1

Compounds of Table B1, and intermediates in the synthesis thereto, were prepared according to experimental procedures detailed in the Example section of WO 2021/091982, which is incorporated herein by reference in its entirety.

### Example 13. Biological Assay Data for Compounds of Table B1

Compounds of Table B1 were tested in the following biological assays as described in detail in WO 2021/091982: decrease in cellular pERK; determination of cell viability in RAS mutant cancer cell lines; disruption of B-Raf Ras-binding domain (BRAF^{RBD}) interaction with K-Ras; in vitro cell proliferation panels; in vivo NSCLC K-Ras G12C xenograft models; and a cell proliferation assay. Certain compounds were also tested in a matched-pair analysis, wherein a H was replaced with (S)Me in the context of two different cell-based assays.

The corresponding data for compounds of Table B1 evaluated in the assays described above are given in Tables 4-19, FIG. 1A, FIG. 1B, FIG. 2A, and FIG. 2B, FIG. 3A, FIG. 3B, FIG. 4A, FIG. 4B, FIG. 5, and the Examples section of WO 2021/091982.

### Example 14. Synthesis of Compounds of Table C1

Compounds of Table C1, and intermediates in the synthesis thereto, were prepared according to experimental procedures detailed in the Example section of WO 2021/091967, which is incorporated herein by reference in its entirety.

### Example 15. Biological Assay Data for Compounds of Table C1

Compounds of Table C1 were tested in the following biological assays as described in detail in WO 2021/091967: decrease in cellular pERK; determination of cell viability in RAS mutant cancer cell lines; disruption of B-Raf Ras-binding domain (BRAF^{BRD}) interaction with K-Ras; cross-linking of Ras proteins with compounds to form conjugates; in vitro cell proliferation panels; and in vivo pharmacodynamics and efficacy.

The corresponding date for compounds of Table C1 evaluated in the assays described above are given in Tables 5-20, FIG. 1A, FIG. 1B, and the Examples section of WO 2021/091967.

### Example 16. Synthesis of Compounds of Table D1

Compounds of Table D1, and intermediates in the synthesis thereto, were prepared according to experimental procedures detailed in the Example section of WO 2022/060836, which is incorporated herein by reference in its entirety.

### Example 17. Biological Assay Data for Compounds of Table D1

Compounds of Table D1 were tested in the following biological assays as described in detail in WO 2022/060836: decrease in cellular pERK; disruption of B-Raf Ras-binding domain (BRAF^{BRD}) interaction with K-Ras; determination of cell viability in RAS mutant cancer cell lines; regressions of KRAD^{G12D} tumors in vivo; regulation of RAS pathway and regressions of KRAS^{G12V} tumors in vivo; regressions of KRAS^{G12V} pancreatic ductal adenocarcinoma and colorectal tumors in vivo; in vivo inhibition of multiple RAS-driven cancer call lines; regressions of KRAS^{G12D} tumors in vivo; regulation of immune checkpoint proteins in NCI-H358, SW900, and Capan-2 cells in vitro; activity against RAS oncogene switching mutations; regressions of a syngenic KRAS G12C tumor model in vivo and synergy with anti-PD-1; modulation of the immune tumor microenvironment in favor of anti-tumor immunity in vivo; anti-tumor activity in KRAS^{G12X} tumor models in vivo; extension of time to tumor doubling across xenograft models; regressions of KRAS^{G12V} tumors in vivo; and inhibition of RAS pathway signaling in vivo.

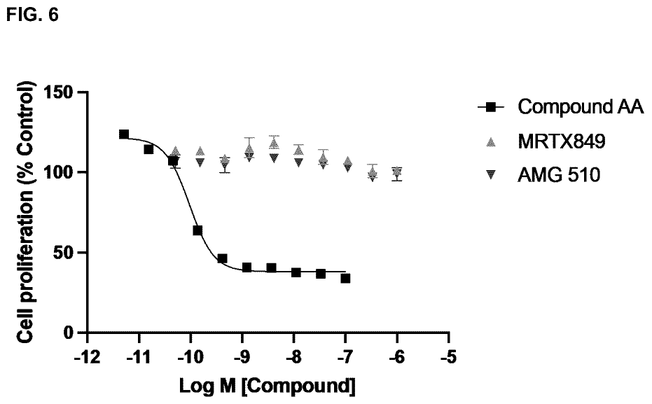

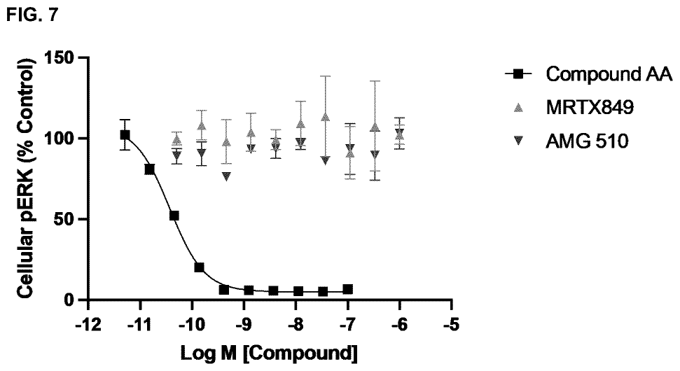

The corresponding data for compounds of Table D1 evaluated in the assays described above are given in Table 5, FIG. 1A, FIG. 11B, FIG. 1C, FIG. 1D, FIG. 1E, FIG. 1F, FIG. 2A, FIG. 2B, FIG. 2C, FIG. 2D, FIG. 2E, FIG. 2F, FIG. 3A, FIG. 3B, FIG. 3C, FIG. 4A, FIG. 4B, FIG. 4C, FIG. 4D, FIG. 4E, FIG. 4F, FIG. 5A, FIG. 5B, FIG. 5C, FIG. 6A-6B, FIG. 7A-7D, FIG. 8, FIG. 9A, FIG. 9B, FIG. 9C, FIG. 10A, FIG. 10B, FIG. 11, FIG. 12A, FIG. 12B, FIG. 12C, FIG. 13A, FIG. 13B, and the Examples section of WO 2022/060836, which is incorporated herein by reference in its entirety.

### OTHER EMBODIMENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims. Other embodiments are within the claims.

1. A method of treating cancer in a subject in need thereof, wherein the cancer comprises a mutation in RAS and the cancer is resistant to treatment with a RAS(OFF) inhibitor, the method comprising administering to the subject a RAS(ON) inhibitor.

2. The method of claim 1, further comprising administering to the subject a RAS(OFF) inhibitor.

3. The method of claim 2, wherein the RAS(ON) inhibitor and the RAS(OFF) inhibitor are administered simultaneously or sequentially.

4. The method of claim 2 or 3 wherein the RAS(ON) inhibitor and the RAS(OFF) inhibitor are administered as a single formulation or in separate formulations.

5. The method of claim 3, wherein:
the RAS(OFF) inhibitor is administered for a first period of time; and
the RAS(ON) inhibitor is administered for a second period of time,
wherein the first period of time and the second period of time do not overlap and the first period of time precedes the second period of time.

6. The method of claim 3, wherein:
the RAS(OFF) inhibitor is administered for a first period of time; and
the RAS(OFF) inhibitor and RAS(ON) inhibitor are administered for a second period of time,
wherein the first period of time and the second period of time do not overlap and the first period of time precedes the second period of time.

7. The method of any one of claims 2-6, wherein the subject's cancer progresses on the RAS(OFF) inhibitor.

8. The method of claim 1, wherein the RAS mutation is an amino acid substitution at Y96.

9. The method of claim 8, wherein the amino acid substitution is Y96D.

10. The method of any one of claims 1, 8, or 9, wherein the subject has been treated with a RAS(OFF) inhibitor.

11. A method of treating cancer in a subject in need thereof, wherein the cancer comprises an amino acid substitution at RAS Y96, the method comprising administering to the subject a RAS(ON) inhibitor.

12. The method of claim 11, wherein the amino acid substitution is Y96D.

13. The method of claim 11 or 12, wherein the subject has been treated with a RAS(OFF) inhibitor.

14. The method of any one of claims 11-13, wherein the cancer is resistant to treatment with a RAS(OFF) inhibitor.

15. The method of claim 13, wherein the subject's cancer progresses on the RAS(OFF) inhibitor.

16. A method of inhibiting RAS in a cell, wherein the RAS comprises an amino acid substitution at Y96, the method comprising contacting the cell with a RAS(ON) inhibitor.

17. The method of claim 16, wherein the amino acid substitution is Y96D.

18. The method of any one of claims 1-17, wherein the RAS comprises or further comprises an amino acid substitution at G12, G13, Q61, or a combination thereof.

19. The method of claim 18, wherein the amino acid substitution is selected from G12C, G12D, G12V, G13C, G13D, or Q61L.

20. The method of claim 19, wherein the amino acid substitution is G12C.

21. The method of any one of claims 1-17, wherein the RAS is KRAS.

22. The method of claim 21, wherein the KRAS comprises or further comprises an amino acid substitution at G12, G13, Q61, A146, K117, L19, Q22, V14, A59, or a combination thereof.

23. The method of claim 22, wherein the KRAS amino acid substitution is selected from G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, G13V, or a combination thereof.

24. The method of any one of claims 1-17, wherein the RAS is NRAS.

25. The method of claim 24, wherein the NRAS comprises or further comprises an amino acid substitution at G12, G13, Q61, P185, A146, G60, A59, E132, E49, T50, or a combination thereof.

26. The method of claim 25, wherein the NRAS amino acid substitution is selected from Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, A59T, or a combination thereof.

27. The method of any one of claims 1-17, wherein the RAS is HRAS.

28. The method of claim 27, wherein the HRAS comprises or further comprises an amino acid substitution at G12, G13, Q61, K117, A59, A18, D119, A66, A146, or a combination thereof.

29. The method of claim 28, wherein the HRAS amino acid substitution is selected from Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, G12R, or a combination thereof.

30. The method of any one of claims 1-29, wherein the RAS(ON) inhibitor is an inhibitor selective for RAS G12C, G13D, or G12D.

31. The method of any one of claims 1-29, wherein the RAS(ON) inhibitor is a RAS(ON)^{MULTI} inhibitor.

32. The method of any one of claims 1-31, wherein the RAS(ON) inhibitor is a compound of Formula AI: 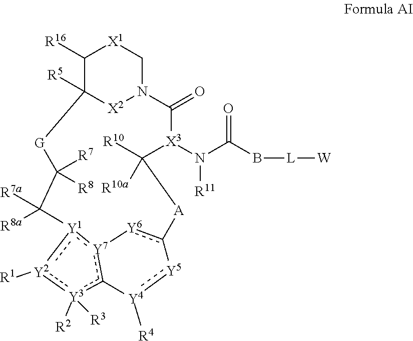
or a pharmaceutically acceptable salt thereof,
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl.

33. The method of any one of claims 1-31, wherein the RAS(ON) inhibitor is selected from a compound of Table A1 or Table A2, or a pharmaceutically acceptable salt thereof.

34. The method of any one of claims 1-31, wherein the RAS(ON) inhibitor is a compound of Formula BI: 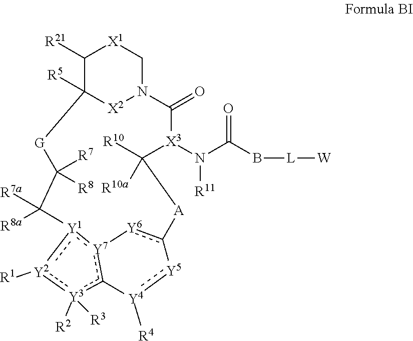
or a pharmaceutically acceptable salt thereof,
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl.

35. The method of any one of claims 1-31, wherein the RAS(ON) inhibitor is selected from a compound of Table B1 or Table B2, or a pharmaceutically acceptable salt thereof.

36. The method of any one of claims 1-31, wherein the RAS(ON) inhibitor is a compound of Formula CI, or a pharmaceutically acceptable salt thereof. 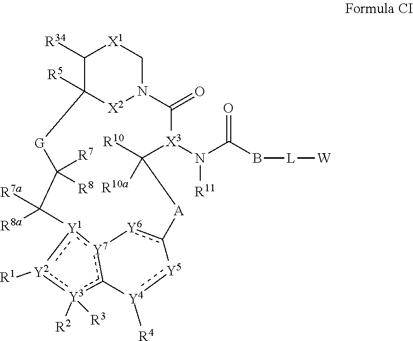
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)—where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl; and
R³⁴ is hydrogen or C₁-C₃ alkyl.

37. The method of any one of claims 1-31, wherein the RAS(ON) inhibitor is selected from a compound of Table C1 or Table C2, or a pharmaceutically acceptable salt thereof.

38. The method of any one of claims 1-31, wherein the RAS(ON) inhibitor is a compound described by Formula DIa: 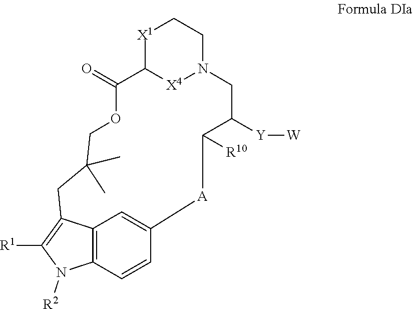
or a pharmaceutically acceptable salt thereof,
wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene; 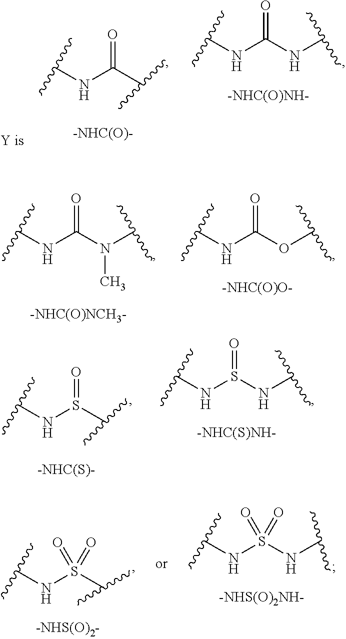
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

39. The method of any one of claims 1-31, wherein the RAS(ON) inhibitor is selected from a compound of Table D1a or D1 b, or a pharmaceutically acceptable salt thereof.

40. The method of any one of claims 1-39, wherein the RAS(OFF) inhibitor selectively targets RAS G12C.

41. The method of any one of claims 1-39, wherein the RAS(OFF) inhibitor selectively targets RAS G12D.

42. The method of any one of claims 1-41, wherein the RAS(OFF) inhibitor is selected from sotorasib (AMG 510), adagrasib (MRTX849), MRTX1257, JNJ-74699157 (ARS-3248), LY3537982, LY3499446, ARS-853, ARS-1620, GDC-6036, JDQ443, BPI-421286, and JAB-21000.

43. The method of any one of claims 1-42, wherein the cancer is selected from colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, bladder cancer, appendiceal cancer, endometrial cancer, and melanoma.

44. The method of claim 43, wherein the cancer is non-small cell lung cancer.

45. The method of claim 43, wherein the cancer is pancreatic cancer.
